(19) United States
(12) Patent Application Publication
(10) Pub. No.: US 2006/0252698 A1
(43) Pub. Date: Nov. 9, 2006

(54) Compounds for inhibiting cathepsin activity

(76) Inventor: Bruce A. Malcolm, Paoli, PA (US)

Correspondence Address: SCHERING-PLOUGH CORPORATION;PATENT DEPARTMENT (K-6-1, 1990), 2000 GALLOPING HILL ROAD, KENILWORTH, NJ, 07033-0530, US

(21) Appl. No.: 11/408,109

(22) Filed: Apr. 20, 2006

Related U.S. Application Data
(60) Provisional application No. 60/673,294, filed on Apr. 20, 2005.

(51) Int. Cl. A61K 38/08

(52) U.S. Cl. 514/17

(57) ABSTRACT

The present invention provides methods of inhibiting cathepsin activity in a subject in need thereof comprising administering to said subject an effective amount of at least one compound of various formulae (e.g., formula I-XXVI) disclosed herein. The present invention also provides methods of treatment of various diseases utilizing the foregoing compounds.

This Application claims the benefit of U.S. Provisional Application Ser. No. 60/673,294 filed Apr. 20, 2004, which is incorporated herein by reference in its entirety.

### FIELD OF THE INVENTION

The present invention relates to compounds and compositions that are useful for treating a wide variety of diseases or disorders-associated with cathepsin activity and for inhibiting cathepsin activity.

### BACKGROUND OF THE INVENTION

Cathepsins (Cats) belong to the papain superfamily of lysosomal cysteine proteases. Cathepsins are involved in the normal proteolysis and turnover of target proteins and tissues as well as in initiating proteolytic cascades by proenzyme activation and in participating in MHC class II molecule expression. Baldwin (1993) Proc. Natl. Acad. Sci., 90: 6796-6800; Mixuochi (1994) Immunol. Lett., 43:189-193.

However, aberrant cathepsin expression has also been implicated in several serious human disease states. Cathepsins have been shown to be abundantly expressed in cancer cells, including breast, lung, prostate, glioblastoma and head/neck cancer cells, (Kos et al. (1998) Oncol. Rep., 5:1349-1361; Yan et al. (1998) Biol. Chem., 379:113-123; Mort et al. (1997) Int. J Biochem. Cell Biol., 29: 715-720; Friedrick et al. (1999) Eur. J Cancer, 35:138-144) and are associated with poor treatment outcome of patients with breast cancer, lung cancer, brain tumor and head/neck cancer. Kos et al, supra. Additionally, aberrant expression of cathepsin is evident in several inflammatory disease states, including rheumatoid arthritis and osteoarthritis. Keyszer (1995) Arthritis Rheum., 38:976-984.

The molecular mechanisms of cathepsin activity are not completely understood. Recently, it was shown that forced expression of cathepsin B rescued cells from serum deprivation-induced apoptotic death (Shibata et al. (1998) Biochem. Biophys. Res. Commun., 251: 199-203) and that treatment of cells with antisense oligonucleotides of cathepsin B induced apoptosis. Isahara et at. (1999) Neuroscience, 91:233-249. These reports suggest an anti-apoptotic role for the cathepsins that is contrary to earlier reports that cathepsins are mediators of apoptosis. Roberts et al (1997) Gastroenterology, 113: 1714-1726; Jones et al. (1998) Am. J Physiol., 275: G723-730.

Cathepsin K is a member of the family of enzymes which are part of the papain superfamily of cysteine proteases. Cathepsins B, H, L, N and S have been described in the literature. Recently, cathepsin K polypeptide and the cDNA encoding such polypeptide were disclosed in U.S. Pat. No. 5,501,969 (called cathepsin O therein). Cathepsin K has been recently expressed, purified, and characterized. Bossard, M. J., et al., (1996) J Biol. Chem. 271, 12517-12524; Drake, F. H., et al., (1996) J. Biol. Chem. 271, 12511-12516; Bromme, D., et al., (1996) J. Biol. Chem. 271, 2126-2132.

Cathepsin K has been variously denoted as cathepsin O, cathepsin X or cathepsin O2 in the literature. The designation cathepsin K is considered to be the more appropriate one (name assigned by Nomenclature Committee of the International Union of Biochemistry and Molecular Biology).

Cathepsins of the papain superfamily of cysteine proteases function in the normal physiological process of protein degradation in animals, including humans, e.g., in the degradation of connective tissue. However, elevated levels of these enzymes in the body can result in pathological conditions leading to disease. Thus, cathepsins have been implicated in various disease states, including but not limited to, infections by pneumocystis carinii, trypsanoma cruzi, trypsanoma brucei brucei, and Crithidia fusiculata; as well as in schistosomiasis malaria, tumor metastasis, metachromatic leukodystrophy, muscular dystrophy, amytrophy, and the like. See International Publication Number WO 94/04172, published on Mar. 3, 1994, and references cited therein. See also European Patent Application EP 0 603 873 A1, and references cited therein. Two bacterial cysteine proteases from P. gingivallis, called gingipains, have been implicated in the pathogenesis of gingivitis. Potempa, J., et al. (1994) Perspectives in Drug Discovery and Design, 2, 445-458.

Cathepsin K is believed to play a causative role in diseases of excessive bone or cartilage loss. Bone is composed of a protein matrix in which spindle- or plate-shaped crystals of hydroxyapatite are incorporated. Type I Collagen represents the major structural protein of bone comprising approximately 90% of the structural protein. The remaining 10% of matrix is composed of a number of non-collagenous proteins, including osteocalcin, proteoglycans, osteopontin, osteonectin, thrombospondin, fibronectin, and bone sialoprotein. Skeletal bone undergoes remodeling at discrete foci throughout life. These foci, or remodeling units, undergo a cycle consisting of a bone resorption phase followed by a phase of bone replacement.

Bone resorption is carried out by osteoclasts, which are multinuclear cells of hematopoietic lineage. The osteoclasts adhere to the bone surface and form a tight sealing zone, followed by extensive membrane ruffling on their apical (i.e., resorbing) surface. This creates an enclosed extracellular compartment on the bone surface that is acidified by proton pumps in the ruffled membrane, and into which the osteoclast secretes proteolytic enzymes. The low pH of the compartment dissolves hydroxyapatite crystals at the bone surface, while the proteolytic enzymes digest the protein matrix. In this way, a resorption lacuna, or pit, is formed. At the end of this phase of the cycle, osteoblasts lay down a new protein matrix that is subsequently mineralized. In several disease states, such as osteoporosis and Paget's disease, the normal balance between bone resorption and formation is disrupted, and there is a net loss of bone at each cycle. Ultimately, this leads to weakening of the bone and may result in increased fracture risk with minimal trauma.

The abundant selective expression of cathepsin K in osteoclasts strongly suggests that this enzyme is essential for bone resorption. Thus, selective inhibition of cathepsin K may provide an effective treatment for diseases of excessive bone loss, including, but not limited to, osteoporosis, gingival diseases such as gingivitis and periodontitis, Paget's disease, hypercalcemia of malignancy, and metabolic bone disease. Cathepsin K levels have also been demonstrated to be elevated in chondroclasts of osteoarthritic synovium. Thus, selective inhibition of cathepsin K may also be useful for treating diseases of excessive cartilage or matrix degradation, including, but not limited to, osteoarthritis and rheumatoid arthritis. Metastatic neoplastic cells also typically express high levels of proteolytic enzymes that degrade the surrounding matrix. Thus, selective inhibition of cathepsin K may also be useful for treating certain neoplastic diseases.

There are reports in the literature of the expression of Cathepsin B and L antigen and that activity is associated with early colorectal cancer progression. Troy et al., (2004) Eur J Cancer, 40(10):1610-6. The findings suggest that cysteine proteases play an important role in colorectal cancer progression.

Cathepsin L has been shown to be an important protein mediating the malignancy of gliomas and it has been suggested that its inhibition may diminish their invasion and lead to increased tumor cell apoptosis by reducing apoptotic threshold. Levicar et al., (2003) Cancer Gene Ther., 10(2): 141-51.

Katunama et al., (2002) Arch Biochem Biophys., 397(2):305-11 reports on antihypercalcemic and antimetastatic effects of CLIK-148 in vivo, which is a specific inhibitor of cathepsin L. This reference also reports that CLIK-148 treatment reduced distant bone metastasis to the femur and tibia of melanoma A375 tumors implanted into the left ventricle of the heart.

Rousselet et al., (2004) Cancer Res., 64(1): 146-51 reports that anti-cathepsin L single chain variable fragment (ScFv) could be used to inhibit the tumorigenic and metastatic phenotype of human melanoma, depending on procathepsin L secretion, and the possible use of anti-cathepsin L ScFv as a molecular tool in a therapeutic cellular approach.

Colella et al., (2003) Biotech Histochem., 78(2):101-8 reports that the cysteine proteinases cathepsin L and B participate in the invasive ability of the PC3 prostrate cancer cell line, and the potential of using cystein protease inhibitiors such as cystatins as anti-metastatic agents.

Krueger et al., (2001) Cancer Gene Ther., 8(7):522-8 reports that in human osteosarcoma cell line MNNG/HOS, cathepsin L influences cellular malignancy by promoting migration and basement membrane degradation.

Frohlich et al., (2204) Arch Dermatol Res., 295(10):411-21 reports that cathepsins B and L are involved in invasion of basal cell carcinoma (BCC) cells.

Cathepsins therefore are attractive targets for the discovery of novel chemotherapeutics effective against a variety of diseases. There is a need for compounds useful in the inhibition of cathepsin activity and in the treatment of these disorders.

### SUMMARY OF THE INVENTION

The present invention provides a method of inhibiting cathepsin activity in a subject in need thereof comprising administering to said subject an effective amount of at least one compound of various structural formulae set forth below.

In one embodiment, the compound is a compound of structural formula I

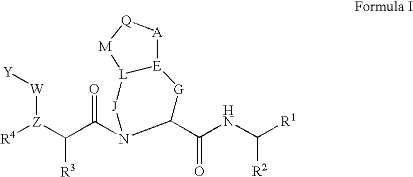

or a pharmaceutically acceptable salt, solvate or ester thereof;

wherein:

Y is selected from the group consisting of the following moieties: alkyl, alkyl-aryl, heteroalkyl, heteroaryl, aryl-heteroaryl, alkyl-heteroaryl, cycloalkyl, alkyloxy, alkyl-aryloxy, aryloxy, heteroaryloxy, heterocycloalkyloxy, cycloalkyloxy, alkylamino, arylamino, alkyl-arylamino, arylamino, heteroarylamino, cycloalkylamino and heterocycloalkylamino, with the proviso that Y maybe optionally substituted with X¹¹ or X¹²;

X¹¹ is alkyl, alkenyl, alkynyl, cycloalkyl, cycloalkyl-alkyl, heterocyclyl, heterocyclylalkyl, aryl, alkylaryl, arylalkyl, heteroaryl, alkylheteroaryl, or heteroarylalkyl, with the proviso that X¹¹ may be additionally optionally substituted with X¹²;

X¹² is hydroxy, alkoxy, aryloxy, thio, alkylthio, arylthio, amino, alkylamino, arylamino, alkylsulfonyl, arylsulfonyl, alkylsulfonamido, arylsulfonamido, carboxy, carbalkoxy, carboxamido, alkoxycarbonylamino, alkoxycarbonyloxy, alkylureido, arylureido, halogen, cyano, or nitro, with the proviso that said alkyl, alkoxy, and aryl may be additionally optionally substituted with moieties independently selected from X¹²;

R¹ is COR⁵ or B(OR)₂, wherein R⁵ is H, OH, OR⁸, NR⁹R¹⁰, CF₃, C₂F₅, C₃F₇, CF₂R⁶, R⁶, or COR⁷ wherein R⁷ is H, OH, OR⁸, CHR⁹R¹⁰, or NR⁹R¹⁰, wherein R⁶, R⁸, R⁹ and R¹⁰ are independently selected from the group consisting of H, alkyl, aryl, heteroalkyl, heteroaryl, cycloalkyl, cycloalkyl, arylalkyl, heteroarylalkyl, [CH(R^{1′})]ₚCOOR¹¹, [CH(R^{1′})]ₚCONR¹²R¹³, [CH(R^{1′})]ₚSO₂R¹¹, [CH(R^{1′})]ₚCOR¹¹, [CH(R^{1′})]ₚCH(OH)RCH(R^{1′})CONHCH(R^{2′})COO R¹¹, CH(R^{1′})CONHCH(R^{2′})CONR¹²R¹³, CH(R^{1′})CONHCH(R^{2′}R′, CH(R^{1′})CONHCH(R^{2′})CONHCH(R^{3′})COOR¹¹, CH(R^{1′})CONHCH(R^{2′})CONHCH(R^{3′}) CONR¹²R¹³, CH(R^{1′})CONHCH(R^{2′})CONHCH(R^{3′})CONHCH(R^{4′})COOR¹¹, CH(R^{1′})CONHCH(R^{2′}) CONHCH(R^{3′})CONHCH(R^{4′})CONR¹²R¹³, CH(R^{1′})CONHCH(R^{2′})CONHCH(R^{3′})CONHCH(R^{4′})CONHCH(R^{5′})COOR¹¹ and CH(R^{1′})CONHCH(R^{2′})CONHCH(R^{3′})CONHCH(R^{4′})CONHCH(R^{5′})CONR¹²R¹³, wherein R^{1′}, R^{2′}, R^{3′}, R^{4′}, R^{5′}, R¹¹, R¹², R¹³, and R′ are independently selected from the group consisting of H, alkyl, aryl, heteroalkyl, heteroaryl, cycloalkyl, alkyl-aryl, alkyl-heteroaryl, aryl-alkyl and heteroaralkyl;

Z is selected from O, N, CH or CR;

W maybe present or absent, and if W is present, W is selected from C═O, C═S, C(═N—OCN), or SO₂;

Q maybe present or absent, and when Q is present, Q is CH, N, P, (CH₂)ₚ, (CHR)ₚ, (CRR′)ₚ, O, NR, S, or SO₂; and when Q is absent, M may be present or absent;

when Q and M are absent, A is directly linked to L;

A is O, CH₂, (CHR)ₚ, (CHR—CHR′)ₚ, (CRR′)ₚ, NR, S, SO₂ or a bond;

E is CH, N, CR, or a double bond towards A, L or G;

G may be present or absent, and when G is present, G is (CH₂)ₚ, (CHR)ₚ, or

(CRR′)ₚ; and when G is absent, J is present and E is directly connected to the carbon atom in Formula I as G is linked to;

J maybe present or absent, and when J is present, J is (CH₂)ₚ, (CHR)ₚ, or (CRR′)ₚ,

SO₂, NH, NR or O; and when J is absent, G is present and E is directly linked to N shown in Formula I as linked to J;

L may be present or absent, and when L is present, L is CH, CR, O, S or NR; and

when L is absent, then M may be present or absent; and if M is present with L being absent, then M is directly and independently linked to E, and J is directly and independently linked to E;

M may be present or absent, and when M is present, M is O, NR, S, SO₂, (CH₂)ₚ, (CHR)ₚ(CHR—CHR′)ₚ, or (CRR′)ₚ;

p is a number from 0 to 6; and

R, R′, R², R³ and R⁴ are independently selected from the group consisting of H; C₁-C₁₀ alkyl; C₂-C₁₀ alkenyl; C₃-C₈ cycloalkyl; C₃-C₈ heterocycloalkyl, alkoxy, aryloxy, alkylthio, arylthio, amino, amido, ester, carboxylic acid, carbamate, urea, ketone, aldehyde, cyano, nitro, halogen; (cycloalkyl)alkyl and (heterocycloalkyl)alkyl, wherein said cycloalkyl is made of three to eight carbon atoms, and zero to six oxygen, nitrogen, sulfur, or phosphorus atoms, and said alkyl is of one to six carbon atoms; aryl; heteroaryl; alkyl-aryl; and alkyl-heteroaryl;

wherein said alkyl, heteroalkyl, alkenyl, heteroalkenyl, aryl, heteroaryl, cycloalkyl and heterocycloalkyl moieties may be optionally and chemically-suitably substituted, with said term “substituted” referring to optional and chemically-suitable substitution with one or more moieties selected from the group consisting of alkyl, alkenyl, alkynyl, aryl, aralkyl, cycloalkyl, heterocyclic, halogen, hydroxy, thio, alkoxy, aryloxy, alkylthio, arylthio, amino, amido, ester, carboxylic acid, carbamate, urea, ketone, aldehyde, cyano, nitro, sulfonamido, sulfoxide, sulfone, sulfonyl urea, hydrazide, and hydroxamate;

further wherein said unit N—C-G-E-L-J-N represents a five-membered or six-membered cyclic ring structure with the proviso that when said unit N—C-G-E-L-J-N represents a five-membered cyclic ring structure, or when the bicyclic ring structure in Formula I comprising N, C, G, E, L, J, N, A, Q, and M represents a five-membered cyclic ring structure, then said five-membered cyclic ring structure lacks a carbonyl group as part of the cyclic ring.

In another embodiment, the compound is a compound of formula II:

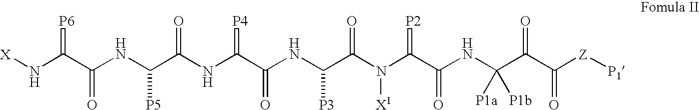

or a pharmaceutically acceptable salt, solvate or ester thereof; wherein:

Z is O, NH or NR^{12;}

X is alkylsulfonyl, heterocyclylsulfonyl, heterocyclylalkylsulfonyl, arylsulfonyl, heteroarylsulfonyl, alkylcarbonyl, heterocyclylcarbonyl, heterocyclylalkylcarbonyl, arylcarbonyl, heteroarylcarbonyl, alkoxycarbonyl, heterocyclyloxycarbonyl, aryloxycarbonyl, heteroaryloxycarbonyl, alkyaminocarbonyl, heterocyclylaminocarbonyl, arylaminocarbonyl, or heteroarylaminocarbonyl moiety, with the proviso that X may be additionally optionally substituted with R or R ; X¹ is H; C₁-C₄ straight chain alkyl; C₁-C₄ branched alkyl or ; CH₂-aryl (substituted or unsubstituted);

R¹² is alkyl, alkenyl, alkynyl, cycloalkyl, cycloalkyl-alkyl, heterocyclyl, heterocyclylalkyl, aryl, alkylaryl, arylalkyl, heteroaryl, alkylheteroaryl, or heteroarylalkyl moiety, with the proviso that R¹² may be additionally optionally substituted with R¹³.

R is hydroxy, alkoxy, aryloxy, thio, alkylthio, arylthio, amino, alkylamino, arylamino, alkylsulfonyl, arylsulfonyl, alkylsulfonamido, arylsulfonamido, carboxy, carbalkoxy, carboxamido, alkoxycarbonylamino, alkoxycarbonyloxy, alkylureido, arylureido, halogen, cyano, or nitro moiety, with the proviso that the alkyl, alkoxy, and aryl may be additionally optionally substituted with moieties independently selected from R¹³.

P1a, P1b, P2, P3, P4, P5, and P6 are independently:

H; C1-C10 straight or branched chain alkyl; C2-C10 straight or branched chain alkenyl;

C3-C8 cycloalkyl, C3-C8 heterocyclic; (cycloalkyl)alkyl or (heterocyclyl)alkyl, wherein said cycloalkyl is made up of 3 to 8 carbon atoms, and zero to 6 oxygen, nitrogen, sulfur, or phosphorus atoms, and said alkyl is of 1 to 6 carbon atoms;

aryl, heteroaryl, arylalkyl, or heteroarylalkyl, wherein said alkyl is of 1 to 6 carbon atoms;

wherein said alkyl, alkenyl, cycloalkyl, heterocyclyl; (cycloalkyl)alkyl and (heterocyclyl)alkyl moieties may be optionally substituted with R¹³, and further wherein said P1a and P1b may optionally be joined to each other to form a spirocyclic or spiroheterocyclic ring, with said spirocyclic or spiroheterocyclic ring containing zero to six oxygen, nitrogen, sulfur, or phosphorus atoms, and may be additionally optionally substituted with R¹³; and

P1′ is H, alkyl, alkenyl, alkynyl, cycloalkyl, cycloalkyl-alkyl, heterocyclyl, heterocyclyl-alkyl, aryl, aryl-alkyl, heteroaryl, or heteroaryl-alkyl; with the proviso that said P1′ may be additionally optionally substituted with R¹³.

In another embodiment, the compound is a compound of formula III

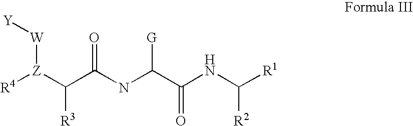

or a pharmaceutically acceptable salt, solvate or ester thereof; wherein:

G, J and Y may be the same or different and are independently selected from the group consisting of the moieties: H, alkyl, alkyl-aryl, heteroalkyl, heteroaryl, aryl-heteroaryl, alkyl-heteroaryl, cycloalkyl, alkyloxy, alkyl-aryloxy, aryloxy, heteroaryloxy, heterocycloalkyloxy, cycloalkyloxy, alkylamino, arylamino, alkyl-arylamino, arylamino, heteroarylamino, cycloalkylamino and heterocycloalkylamino, with the proviso that Y maybe additionally optionally substituted with X¹¹ or X¹²;

X¹¹ is selected from the group consisting of alkyl, alkenyl, alkynyl, cycloalkyl, cycloalkyl-alkyl, heterocyclyl, heterocyclylalkyl, aryl, alkylaryl, arylalkyl, heteroaryl, alkylheteroaryl, or heteroarylalkyl moiety, with the proviso that X¹¹ may be additionally optionally substituted with X¹²;

X¹² is hydroxy, alkoxy, aryloxy, thio, alkylthio, arylthio, amino, alkylamino, arylamino, alkylsulfonyl, arylsulfonyl, alkylsulfonamido, arylsulfonamido, carboxy, carbalkoxy, carboxamido, alkoxycarbonylamino, alkoxycarbonyloxy, alkylureido, arylureido, halogen, cyano, or nitro, with the proviso that said alkyl, alkoxy, and aryl may be additionally optionally substituted with moieties independently selected from X¹²;

R¹ is COR⁵ or B(OR)₂, wherein R⁵ is selected from the group consisting of H, OH, OR⁸, NR⁹R¹⁰, CF₃, C₂F₅, C₃F₇, CF₂R⁶, R⁶ and COR⁷ wherein R⁷ is selected from the group consisting of H, OH, OR⁸, CHR⁹R¹⁰, and NR⁹R¹⁰, wherein R⁶, R⁸, R⁹ and R¹⁰ may be the same or different and are independently selected from the group consisting of H, alkyl, aryl, heteroalkyl, heteroaryl, cycloalkyl, cycloalkyl, arylalkyl, heteroarylalkyl, CH(R^{1′})COOR¹¹, CH(R^{1′})CONR¹²R¹³, CH(R^{1′})CONHCH(R^{2′})COOR¹¹, CH(R^{1′})CONHCH(R^{2′})CONR¹²R¹³, CH(R^{1′})CONHCH(R^{2!})R′, CH(R^{1′})CONHCH(R^{2′})CO NHCH(R^{3′})COOR¹¹, CH(R^{1′})CONHCH(R^{2′})CONHCH(R^{3′})CONR¹²R¹³, CH(R^{1′})CONHCH(R^{2′})CONHCH(R^{3′})CONHCH(R^{4′})COOR¹¹, CH(R^{1′})CONHCH(R^{2′})CONHCH(R^{3′})CONHCH(R^{4′})CONR¹²R¹³, CH(R^{1′})CONHCH(R^{2′})CONHCH(R^{3′})CONHCH(R^{4′})CONHCH(R^{5′})COOR¹¹, and CH(R^{1′})CONHCH(R^{2′})CONHCH(R^{3′})CONHCH(R^{4′})CONHCH(R^{5′}) CONR¹²R¹³, wherein R^{1′}, R^{2′}, R^{3′}, R^{4′}, R^{5′}, R¹¹, R¹², R¹³, and R′πmay be the same or different and are independently selected from a group consisting of H, alkyl, aryl, heteroalkyl, heteroaryl, cycloalkyl, alkyl-aryl, alkyl-heteroaryl, aryl-alkyl and heteroaralkyl;

Z is selected from O, N, or CH;

W maybe present or absent, and if W is present, W is selected from C═O, C═S, or SO₂; and

R, R′, R², R³ and R⁴ are independently selected from the group consisting of H; C1-C10 alkyl; C2-C10 alkenyl; C3-C8 cycloalkyl; C3-C8 heterocycloalkyl, alkoxy, aryloxy, alkylthio, arylthio, amino, amido, ester, carboxylic acid, carbamate, urea, ketone, aldehyde, cyano, nitro; oxygen, nitrogen, sulfur, or phosphorus atoms (with said oxygen, nitrogen, sulfur, or phosphorus atoms numbering zero to six); (cycloalkyl)alkyl and (heterocycloalkyl)alkyl, wherein said cycloalkyl is made of three to eight carbon atoms, and zero to six oxygen, nitrogen, sulfur, or phosphorus atoms, and said alkyl is of one to six carbon atoms; aryl; heteroaryl; alkyl-aryl; and alkyl-heteroaryl;

wherein said alkyl, heteroalkyl, alkenyl, heteroalkenyl, aryl, heteroaryl, cycloalkyl and heterocycloalkyl moieties may be optionally substituted, with said term “substituted” referring to optional and chemically-suitable substitution with one or more moieties selected from the group consisting of alkyl, alkenyl, alkynyl, aryl, aralkyl, cycloalkyl, heterocyclic, halogen, hydroxy, thio, alkoxy, aryloxy, alkylthio, arylthio, amino, amido, ester, carboxylic acid, carbamate, urea, ketone, aldehyde, cyano, nitro, sulfonamide, sulfoxide, sulfone, sulfonylurea, hydrazide, and hydroxamate.

In another embodiment, the compound is a compound of formula IV

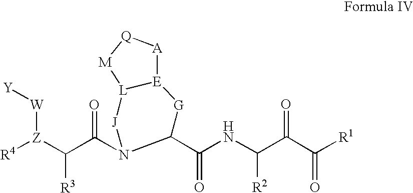

or a pharmaceutically acceptable salt, solvate or ester thereof; wherein:

Y is selected from the group consisting of the following moieties: alkyl, alkyl-aryl, heteroalkyl, heteroaryl, aryl-heteroaryl, alkyl-heteroaryl, cycloalkyl, alkyloxy, alkyl-aryloxy, aryloxy, heteroaryloxy, heterocycloalkyloxy, cycloalkyloxy, alkylamino, arylamino, alkyl-arylamino, arylamino, heteroarylamino, cycloalkylamino and heterocycloalkylamino, with the proviso that Y maybe optionally substituted with X¹¹ or X¹²;

X¹¹ is alkyl, alkenyl, alkynyl, cycloalkyl, cycloalkyl-alkyl, heterocyclyl, heterocyclylalkyl, aryl, alkylaryl, arylalkyl, heteroaryl, alkylheteroaryl, or heteroarylalkyl, with the proviso that X¹¹ may be additionally optionally substituted with X¹²;

X¹² is hydroxy, alkoxy, aryloxy, thio, alkylthio, arylthio, amino, alkylamino, arylamino, alkylsulfonyl, arylsulfonyl, alkylsulfonamido, arylsulfonamido, carboxyl, carbalkoxy, carboxamido, alkoxycarbonylamino, alkoxycarbonyloxy, alkylureido, arylureido, halogen, cyano, or nitro, with the proviso that said alkyl, alkoxy, and aryl may be additionally optionally substituted with moieties independently selected from X¹²;

R¹ is selected from the following structures:

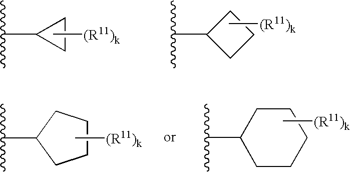

wherein k is a number from 0 to 5, which can be the same or different, R¹¹ denotes optional substituents, with each of said substituents being independently selected from the group consisting of alkyl, alkenyl, alkynyl, aryl, cycloalkyl, alkyl-aryl, heteroalkyl, heteroaryl, aryl-heteroaryl, alkyl-heteroaryl, alkyloxy, alkyl-aryloxy, aryloxy, heteroaryloxy, heterocycloalkyloxy, cycloalkyloxy, alkylamino, arylamino, alkyl-arylamino, arylamino, heteroarylamino, cycloalkylamino, heterocycloalkylamino, hydroxy, thio, alkylthio, arylthio, amino, alkylsulfonyl, arylsulfonyl, alkylsulfonamido, arylsulfonamido, carboxyl, carbalkoxy, carboxamido, alkoxycarbonylamino, alkoxycarbonyloxy, alkylureido, arylureido, halogen, cyano, and nitro, with the proviso that R¹¹ (when R¹¹≠H) maybe optionally substituted with X¹¹ or X¹²;

Z is selected from O, N, CH or CR;

W may be present or absent, and if W is present, W is selected from C═O, C═S, C(═N—CN), or S(O₂);

Q may be present or absent, and when Q is present, Q is CH, N, P, (CH₂)ₚ, (CHR)ₚ, (CRR′)ₚ, O, N(R), S, or S(O₂); and when Q is absent, M may be present or absent;

when Q and M are absent, A is directly linked to L;

A is O, CH₂, (CHR)ₚ, (CHR—CHR′)ₚ, (CRR′)ₚ, N(R), S, S(O₂) or a bond;

E is CH, N, CR, or a double bond towards A, L or G;

G may be present or absent, and when G is present, G is (CH₂)ₚ, (CHR)ₚ, or (CRR′)ₚ; and when G is absent, J is present and E is directly connected to the carbon atom in Formula I as G is linked to;

J may be present or absent, and when J is present, J is (CH₂)ₚ, (CHR)ₚ, or (CRR′)ₚ, S(O₂), NH, N(R) or O; and when J is absent, G is present and E is directly linked to N shown in Formula I as linked to J;

L may be present or absent, and when L is present, L is CH, C(R), O, S or N(R); and

when L is absent, then M may be present or absent; and if M is present with L being absent, then M is directly and independently linked to E, and J is directly and independently linked to E;

M may be present or absent, and when M is present, M is O, N(R), S, S(O₂), (CH₂)ₚ, (CHR)ₚ(CHR—CHR′)ₚ, or (CRR′)ₚ;

p is a number from 0 to 6; and

R, R′, R², R³ and R⁴ can be the same or different, each being independently selected from the group consisting of H; C₁-C₁₀ alkyl; C₂-C₁₀ alkenyl; C₃-C₈ cycloalkyl; C₃-C₈ heterocycloalkyl, alkoxy, aryloxy, alkylthio, arylthio, amino, amido, ester, carboxylic acid, carbamate, urea, ketone, aldehyde, cyano, nitro, halogen, (cycloalkyl)alkyl and (heterocycloalkyl)alkyl, wherein said cycloalkyl is made of three to eight carbon atoms, and zero to six oxygen, nitrogen, sulfur, or phosphorus atoms, and said alkyl is of one to six carbon atoms; aryl; heteroaryl; alkyl-aryl; and alkyl-heteroaryl;

wherein said alkyl, heteroalkyl, alkenyl, heteroalkenyl, aryl, heteroaryl, cycloalkyl and heterocycloalkyl moieties may be optionally substituted, with said term “substituted” referring to substitution with one or more moieties which can be the same or different, each being independently selected from the group consisting of alkyl, alkenyl, alkynyl, aryl, aralkyl, cycloalkyl, heterocyclic, halogen, hydroxy, thio, alkoxy, aryloxy, alkylthio, arylthio, amino, amido, ester, carboxylic acid, carbamate, urea, ketone, aldehyde, cyano, nitro, sulfonamido, sulfoxide, sulfone, sulfonyl urea, hydrazide, and hydroxamate;

further wherein said unit N—C-G-E-L-J-N represents a five-membered cyclic ring structure or six-membered cyclic ring structure with the proviso that when said unit N—C-G-E-L-J-N represents a five-membered cyclic ring structure, or when the bicyclic ring structure in Formula I comprising N, C, G, E, L, J, N, A, Q, and M represents a five-membered cyclic ring structure, then said five-membered cyclic ring structure lacks a carbonyl group as part of said five-membered cyclic ring.

In another embodiment, the compound is a compound of formula V

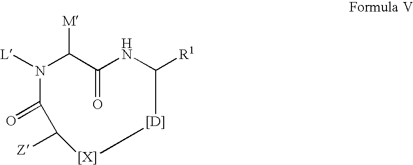

or a pharmaceutically acceptable salt, solvate or ester of said compound wherein:

(1) R¹ is —C(O)R⁵ or —B(OR)₂;

(2) R⁵ is H, —OH, —OR⁸, —NR⁹R¹⁰, —C(O)OR , —C(O)NR⁹R¹⁰, —CF₃, —C₂F₅, C₃F₇, —CF₂R⁶, —R⁶, —C(O)R⁷ or NR⁷SO₂R⁸;

(3) R⁷ is H, —OH, —OR⁹, or —CHR⁹R¹⁰;

(4) R⁶, R⁸, R⁹ and R¹⁰ are independently selected from the group consisting of H: alkyl, alkenyl, aryl, heteroalkyl, heteroaryl, cycloalkyl, arylalkyl, heteroarylalkyl, R¹⁴, —CH(R^{1′})CH(R^{1′})C(O)OR¹¹, [CH(R^{1′})]ₚC(O)OR¹¹, —[CH(R^{1′})]ₚC(O)NR¹²R¹³, —[CH(R^{1′})]ₚS(O₂)R¹¹, —[CH(R^{1′})]ₚC(O)R¹¹, —[CH(R^{1′})]ₚS(O₂)NR¹²R¹³, CH(R^{1′})C(P)N(H)CH(R^{2′})(R′), CH(R^{1′})CH(R^{1′})C(O)NR¹²R¹³, —CH(R^{1′})CH(R^{1′})S(O₂)R¹¹, CH(R^{1′})CH(R^{1′})S(O₂)NR¹²R¹³, —CH(R^{1′})CH(R^{1′})C(O)R¹¹, —[CH(R^{1′})]ₚCH(OH)R¹¹, —CH(R^{1′})C(O)N(H)CH(R^{2′})C(O)OR¹¹, C(O)N(H)CH(R^{2′})C(O)OR¹¹, —C(O)N(H)CH(R^{2′})C(O)R¹¹, CH(R^{1′})C(O)N(H)CH(R^{2′}) C(O)NR¹²R¹³, —CH(R^{1′})C(O)N(H)CH(R^{2′})R′, CH(R^{1′})C(O)N(H)CH(R^{2′})C(O)N(H) CH(R^{3′})C(O)OR¹¹, CH(R^{1′})C(O)N(H)CH(R^{2′})C(O)CH(R^{3′})NR¹²R¹³, CH(R^{1′})C(O)N(H)CH (R^{2′})C(O)N(H)CH(R^{3′})C(O)NR¹²R¹³, CH(R^{1′})C(O)N(H)CH(R^{2′})C(O)N(H)CH(R^{3′})C(O)N(H)CH(R^{4′})C(O)OR¹¹, H(R^{1′})C(O)N(H)CH(R^{2′})C(O)N(H)CH(R^{3′})C(O)N(H)CH(R^{4′})C(O)NR¹²R¹³, CH(R^{1′})C(O)N(H)CH(R^{2′})C(O)N(H)CH(R^{3′})C(O)N(H)CH(R^{4′})C(O)N(H)CH(R^{5′})C(O)OR¹¹, and CH(R^{1′})C(O)N(H)CH(R^{2′})C(O)N(H)CH(R^{3′})C(O)N(H)CH(R^{4′})C(O)N(H)CH(R^{5′})C(O)NR¹²R¹³;

wherein R^{1′}, R^{2′}, R^{3′}, R^{4′}, R^{5′}, R¹¹, R¹²and R¹³ can be the same or different, each being independently selected from the group consisting of: H, halogen, alkyl, aryl, heteroalkyl, heteroaryl, cycloalkyl, alkoxy, aryloxy, alkenyl, alkynyl, alkyl-aryl, alkyl-heteroaryl, heterocycloalkyl, aryl-alkyl and heteroaralkyl;

or

R¹² and R¹³ are linked together wherein the combination is cycloalkyl, heterocycloalkyl, ary or heteroaryl;

R¹⁴ is present or not and if present is selected from the group consisting of: H, alkyl, aryl, heteroalkyl, heteroaryl, cycloalkyl, alkyl-aryl, allyl, alkyl-heteroaryl, alkoxy, aryl-alkyl, alkenyl, alkynyl and heteroaralkyl;

(5) R and R′ are present or not and if present can be the same or different, each being independently selected from the group consisting of: H, OH, C₁-C₁₀ alkyl, C₂-C₁₀ alkenyl, C₃-C₈ cycloalkyl, C₃-C₈ heterocycloalkyl, alkoxy, aryloxy, alkylthio, arylthio, alkylamino, arylamino, amino, amido, arylthioamino, arylcarbonylamino, arylaminocarboxy, alkylaminocarboxy, heteroalkyl, alkenyl, alkynyl, (aryl)alkyl, heteroarylalkyl, ester, carboxylic acid, carbamate, urea, ketone, aldehyde, cyano, nitro, halogen, (cycloalkyl)alkyl, aryl, heteroaryl, (alkyl)aryl, alkylheteroaryl, alkyl-heteroaryl and (heterocycloalkyl)alkyl, wherein said cycloalkyl is made of three to eight carbon atoms, and zero to six oxygen, nitrogen, sulfur, or phosphorus atoms, and said alkyl is of one to six carbon atoms;

(6) L′ is H, OH, alkyl, heteroalkyl, aryl, heteroaryl, cycloalkyl, or heterocyclyl;

(7) M′ is H, alkyl, heteroalkyl, aryl, heteroaryl, cycloalkyl, arylalkyl, heterocyclyl or an amino acid side chain;

or L′ and M′ are linked together to form a ring structure wherein the portion of structural Formula 1 represented by

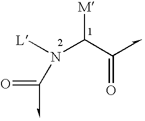

is represented by structural Formula 2:

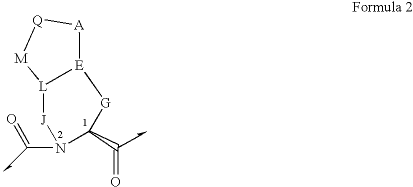

wherein in Formula 2:

E is present or absent and if present is C, CH, N or C(R);

J is present or absent, and when J is present, J is (CH₂)ₚ, (CHR—CHR′)ₚ, (CHR)ₚ, (CRR′)ₚ, S(O₂), N(H), N(R) or O; when J is absent and G is present, L is directly linked to the nitrogen atom marked position 2;

p is a number from 0 to 6;

L is present or absent, and when L is present, L is C(H) or C(R); when L is absent, M is present or absent; if M is present with L being absent, then M is directly and independently linked to E, and J is directly and independently linked to E;

G is present or absent, and when G is present, G is (CH₂)ₚ, (CHR)ₚ, (CHR—CHR′)ₚ or (CRR′)ₚ; when G is absent, J is present and E is directly connected to the carbon atom marked position 1;

Q is present or absent, and when Q is present, Q is NR, PR, (CR═CR), (CH₂)ₚ, (CHR)ₚ, (CRR′)ₚ, (CHR—CHR′)ₚ, O, NR, S, SO, or SO₂; when Q is absent, M is (i) either directly linked to A or (ii) an independent substituent on L, said independent substituent bing selected from —OR, —CH(R)(R′), S(O)₀₋₂R or —NRR′ or (iii) absent; when both Q and M are absent, A is either directly linked to L, or A is an independent substituent on E, said independent substituent bing selected from —OR, —CH(R)(R′), S(O)₀₋₂R or —NRR′ or A is absent;

A is present or absent and if present A is O, O(R), (CH₂)ₚ, (CHR)ₚ, (CHR—CHR′)ₚ, (CRR′)ₚ, N(R), NRR′, S, S(O₂), —OR, CH(R)(R′) or NRR′; or A is linked to M to form an alicyclic, aliphatic or heteroalicyclic bridge;

M is present or absent, and when M is present, M is halogen, O, OR, N(R), S, S(O₂), (CH₂)ₚ, (CHR)ₚ(CHR—CHR′)ₚ, or (CRR′)ₚ; or M is linked to A to form an alicyclic, aliphatic or heteroalicyclic bridge;

(8) Z′ is represented by the structural Formula 3:

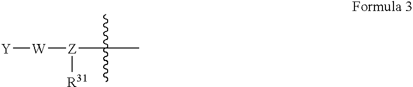

wherein in Formula 3, Y is selected from the group consisting of: H, aryl, alkyl, alkyl-aryl, heteroalkyl, heteroaryl, aryl-heteroaryl, alkyl-heteroaryl, cycloalkyl, alkyloxy, alkyl-aryloxy, aryloxy, heteroaryloxy, heterocycloalkyloxy, heteroalkyl-heteroaryl, heteroalkyl-heterocycloalkyl, cycloalkyloxy, alkylamino, arylamino, alkyl-arylamino, arylamino, heteroarylamino, cycloalkylamino and heterocycloalkylamino, and Y is unsubstituted or optionally substituted with one or two substituents which are the same or different and are independently selected from X¹¹ or X¹²;

X¹¹ is alkyl, alkenyl, alkynyl, cycloalkyl, cycloalkyl-alkyl, heterocyclyl, heterocyclylalkyl, aryl, alkylaryl, arylalkyl, heteroaryl, alkylheteroaryl, or heteroarylalkyl, and X¹ is unsubstituted or optionally substituted with one or more of X¹² moieties which are the same or different and are independently selected;

X¹² is hydroxy, alkoxy, alkyl, alkenyl, alkynyl, aryl, aryloxy, thio, alkylthio, arylthio, amino, alkylamino, arylamino, alkylsulfonyl, arylsulfonyl, alkylsulfonamido, arylsulfonamido, carboxy, carbalkoxy, carboxamido, alkylcarbonyl, arylcarbonyl, heteroalkylcarbonyl, heteroarylcarbonyl, sulfonylurea, cycloalkylsulfonamido, heteroaryl-cycloalkylsulfonamido, heteroaryl-sulfonamido, alkoxycarbonylamino, alkoxycarbonyloxy, alkylureido, arylureido, halogen, cyano, or nitro, and said alkyl, alkoxy, and aryl are unsubstituted or optionally independently substituted with one or more moieties which are the same or different and are independently selected from alkyl, alkenyl, alkynyl, cycloalkyl, cycloalkyl-alkyl, heterocyclyl, heterocyclylalkyl, aryl, alkylaryl, arylalkyl, heteroaryl, alkylheteroaryl, or heteroarylalkyl;

Z is O, N, C(H) or C(R);

R³¹ is H, hydroxyl, aryl, alkyl, alkyl-aryl, heteroalkyl, heteroaryl, aryl-heteroaryl, alkyl-heteroaryl, cycloalkyl, alkyloxy, alkyl-aryloxy, aryloxy, heteroaryloxy, heterocycloalkyloxy, heteroalkyl-heteroaryl, cycloalkyloxy, alkylamino, arylamino, alkyl-arylamino, arylamino, heteroarylamino, cycloalkylamino or heterocycloalkylamino, and R³¹ is unsubstituted or optionally substituted with one or two substituents which are the same or different and are independently selected from X¹³ or X¹⁴;

X¹³ is alkyl, alkenyl, alkynyl, cycloalkyl, cycloalkyl-alkyl, heterocyclyl, heterocyclylalkyl, aryl, alkylaryl, arylalkyl, heteroaryl, alkylheteroaryl, or heteroarylalkyl, and X¹³ is unsubstituted or optionally substituted with one or more of X¹⁴ moieties which are the same or different and are independently selected;

X¹⁴ is hydroxy, alkoxy, alkyl, alkenyl, alkynyl, aryl, aryloxy, thio, alkylthio, arylthio, amino, alkylamino, arylamino, alkylsulfonyl, arylsulfonyl, alkylsulfonamido, arylsulfonamido, carboxy, carbalkoxy, carboxamido, alkylcarbonyl, arylcarbonyl, heteroalkylcarbonyl, heteroarylcarbonyl, cycloalkylsulfonamido, heteroaryl-cycloalkylsulfonamido, heteroarylsulfonamido, alkoxycarbonylamino, alkoxycarbonyloxy, alkylureido, arylureido, halogen, cyano, or nitro, and said alkyl, alkoxy, and aryl are unsubstiuted or optionally independently substituted with one or more moieties which are the same or different and are independently selected from alkyl, alkenyl, alkynyl, cycloalkyl, cycloalkyl-alkyl, heterocyclyl, heterocyclylalkyl, aryl, alkylaryl, arylalkyl, heteroaryl, alkylheteroaryl, or heteroarylalkyl;

W may be present or absent, and if W is present, W is C(═O), C(═S), C(═N-CN), or S(O₂);

(9) X is represented by structural Formula 4:

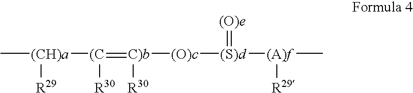

wherein in Formula 4, a is 2, 3, 4, 5, 6, 7, 8 or 9;

b, c, d, e and f are 0, 1, 2, 3, 4 or 5;

A is C, N, S or O;

R²⁹ and R^{29′} are independently present or absent and if present can be the same or different, each being independently one or two substituents independently selected from the group consisting of: H, halo, alkyl, aryl, cycloalkyl, cycloalkylamino, cycloalkylaminocarbonyl, cyano, hydroxy, alkoxy, alkylthio, amino, —NH(alkyl), —NH(cycloalkyl), —N(alkyl)₂, carboxyl, C(O)O-alkyl, heteroaryl, aralkyl, alkylaryl, aralkenyl, heteroaralkyl, alkylheteroaryl, heteroaralkenyl, hydroxyalkyl, aryloxy, aralkoxy, acyl, aroyl, nitro, aryloxycarbonyl, aralkoxycarbonyl, alkylsulfonyl, arylsulfonyl, heteroarylsulfonyl, alkylsulfinyl, arylsulfinyl, heteroarylsulfinyl, arylthio, heteroarylthio, aralkylthio, heteroaralkylthio, cycloalkenyl, heterocyclyl, heterocyclenyl, Y₁Y₂N-alkyl-, Y₁Y₂NC(O)— and Y₁Y₂NSO₂—, wherein Y₁ and Y₂ can be the same or different and are independently selected from the group consisting of hydrogen, alkyl, aryl, and aralkyl; or

R²⁹ and R^{29′} are linked together such that the combination is an aliphatic or heteroaliphatic chain of 0 to 6 carbons;

R³⁰ is present or absent and if present is one or two substituents independently selected from the group consisting of: H, alkyl, aryl, heteroaryl and cylcoalkyl;

(10) D is represented by structural Formula 5:

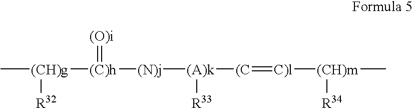

wherein in Formula 5, R³², R³³ and R³⁴ are present or absent and if present are independently one or two substituents independently selected from the group consisting of: H, halo, alkyl, aryl, cycloalkyl, cycloalkylamino, spiroalkyl, cycloalkylaminocarbonyl, cyano, hydroxy, alkoxy, alkylthio, amino, —NH(alkyl), —NH(cycloalkyl), —N(alkyl)₂, carboxyl, —C(O)O-alkyl, heteroaryl, aralkyl, alkylaryl, aralkenyl, heteroaralkyl, alkylheteroaryl, heteroaralkenyl, hydroxyalkyl, aryloxy, aralkoxy, acyl, aroyl, nitro, aryloxycarbonyl, aralkoxycarbonyl, alkylsulfonyl, arylsulfonyl, heteroarylsulfonyl, alkylsulfinyl, arylsulfinyl, heteroarylsulfinyl, arylthio, heteroarylthio, aralkylthio, heteroaralkylthio, cycloalkenyl, heterocyclyl, heterocyclenyl, Y₁Y₂N-alkyl-, Y₁Y₂NC(O)— and Y₁Y₂NSO₂—, wherein Y₁ and Y₂ can be the same or different and are independently selected from the group consisting of hydrogen, alkyl, aryl, and aralkyl; or

R³² and R³⁴ are linked together such that the combination forms a portion of a cycloalkyl group;

g is 1, 2, 3, 4, 5, 6, 7, 8 or 9;

h, i, j, k, l and m are 0, 1, 2, 3, 4 or 5; and

A is C, N, S or O,

(11) provided that when structural Formula 2:

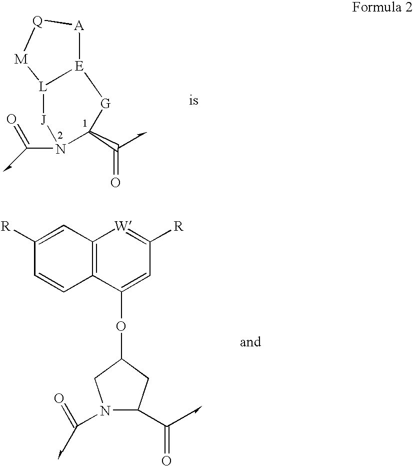

W′ is CH or N, both the following conditional exclusions (i) and (ii) apply: conditional exclusion (i): Z′ is not —NH—R³⁶, wherein R³⁶ is H, C_{6 or 10} aryl, heteroaryl, —C(O)—R³⁷, —C(O)—OR³⁷ or —C(O)—NHR³⁷, wherein R³⁷ is C₁₋₆ alkyl or C₃₋₆ cycloalkyl;

and

conditional exclusion (ii): R¹ is not —C(O)OH, a pharmaceutically acceptable salt of —C(O)OH, an ester of —C(O)OH or —C(O)NHR³⁸ wherein R³⁸ is selected from the group consisting of C₁₋₈ alkyl, C₃₋₆ cycloalkyl, C_{6 to 10} aryl or C₇₋₁₆ aralkyl.

In another embodiment, the compound is a compound of formula VI

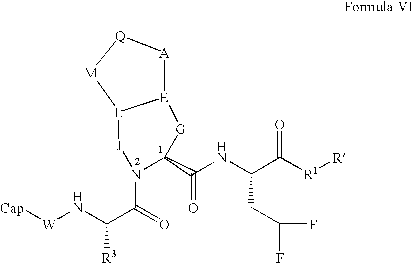

or a pharmaceutically acceptable salt, solvate or ester of said compound, wherein: Cap and P′ are independently H, alkyl, alkyl-aryl, heteroalkyl, heteroaryl, aryl-heteroaryl, alkyl-heteroaryl, cycloalkyl, alkyloxy, alkyl-aryloxy, aryloxy, heteroaryloxy, heterocyclyloxy, cycloalkyloxy, amino, alkylamino, arylamino, alkyl-arylamino, arylamino, heteroarylamino, cycloalkylamino, carboxyalkylamino, arlylalkyloxy or heterocyclylamino, wherein each of said alkyl, alkyl-aryl, heteroalkyl, heteroaryl, aryl-heteroaryl, alkyl-heteroaryl, cycloalkyl, alkyloxy, alkyl-aryloxy, aryloxy, heteroaryloxy, heterocyclyloxy, cycloalkyloxy, amino, alkylamino, arylamino, alkyl-arylamino, arylamino, heteroarylamino, cycloalkylamino, carboxyalkylamino, arlylalkyloxy or heterocyclylamino can be unsubstituted or optionally independently substituted with one or two substituents which can be the same or different and are independently selected from X¹ and X²;

X¹ is alkyl, alkenyl, alkynyl, cycloalkyl, cycloalkyl-alkyl, heterocyclyl, heterocyclylalkyl, aryl, alkylaryl, arylalkyl, arylheteroaryl, heteroaryl, heterocyclylamino, alkylheteroaryl, or heteroarylalkyl, and X¹ can be unsubstituted or optionally independently substituted with one or more of X² moieties which can be the same or different and are independently selected;

X² is hydroxy, alkyl, aryl, alkoxy, aryloxy, thio, alkylthio, arylthio, amino, alkylamino, arylamino, alkylsulfonyl, arylsulfonyl, alkylsulfonamido, arylsulfonamido, carboxy, carbalkoxy, carboxamido, alkoxycarbonylamino, alkoxycarbonyloxy, alkylureido, arylureido, halogen, cyano, keto, ester or nitro, wherein each of said alkyl, alkoxy, and aryl can be unsubstituted or optionally independently substituted with one or more moieties which can be the same or different and are independently selected from alkyl, alkenyl, alkynyl, cycloalkyl, cycloalkyl-alkyl, heterocyclyl, heterocyclylalkyl, aryl, alkylaryl, arylalkyl, arylheteroaryl, heteroaryl, heterocyclylamino, alkylheteroaryl and heteroarylalkyl;

W may be present or absent, and when W is present W is C(═O), C(═S), C(═NH), C(═N—OH), C(═N—CN), S(O) or S(O₂);

Q maybe present or absent, and when Q is present, Q is N(R), P(R), CR═CR′, (CH₂)ₚ, (CHR)ₚ, (CRR′)ₚ, (CHR—CHR′)ₚ, O, S, S(O) or S(O₂); when Q is absent, M is (i) either directly linked to A or (ii) M is an independent substituent on L and A is an independent substituent on E, with said independent substituent being selected from —OR, —CH(R′), S(O)₀₋₂R or —NRR′; when both Q and M are absent, A is either directly linked to L, or A is an independent substituent on E, selected from —OR, CH(R)(R′), —S(O)₀₋₂R or —NRR′;

A is present or absent and if present A is —O—, —O(R)CH₂—, —(CHR)ₚ—, —(CHR—CHR′)ₚ—, (CRR′)ₚ, N(R), NRR′, S, or S(O₂), and when Q is absent, A is —OR, —CH(R)(R′) or —NRR′; and when A is absent, either 0 and E are connected by a bond or Q is an independent substituent on M;

E is present or absent and if present E is CH, N, C(R);

G may be present or absent, and when G is present, G is (CH₂)ₚ, (CHR)ₚ, or (CRR′)ₚ; when G is absent, J is present and E is directly connected to the carbon atom marked position 1;

J may be present or absent, and when J is present, J is (CH₂)ₚ, (CHR—CHR′)ₚ, (CHR)ₚ, (CRR′)ₚ, S(O₂), N(H), N(R) or O; when J is absent and G is present, L is directly linked to the nitrogen atom marked position 2;

L may be present or absent, and when L is present, L is CH, N, or CR; when L is absent, M is present or absent; if M is present with L being absent, then M is directly and independently linked to E, and J is directly and independently linked to E;

M may be present or absent, and when M is present, M is O, N(R), S, S(O₂), (CH₂)ₚ, (CHR)ₚ, (CHR—CHR′)ₚ, or (CRR′)ₚ;

p is a number from 0 to 6;

R, R′ and R³ can be the same or different, each being independently selected from the group consisting of: H, C₁-C₁₀ alkyl, C₂-C₁₀ alkenyl, C₃-C₈ cycloalkyl, C₃-C₈ heterocyclyl, alkoxy, aryloxy, alkylthio, arylthio, amino, amido, arylthioamino, arylcarbonylamino, arylaminocarboxy, alkylaminocarboxy, heteroalkyl, heteroalkenyl, alkenyl, alkynyl, aryl-alkyl, heteroarylalkyl, ester, carboxylic acid, carbamate, urea, ketone, aldehyde, cyano, nitro, halogen, (cycloalkyl)alkyl, aryl, heteroaryl, alkyl-aryl, alkylheteroaryl, alkyl-heteroaryl and (heterocyclyl)alkyl;

R and R′ in (CRR′) can be linked together such that the combination forms a cycloalkyl or heterocyclyl moiety; and

R¹ is N(R) or O.

In another embodiment, the compound is a compound of formula VII

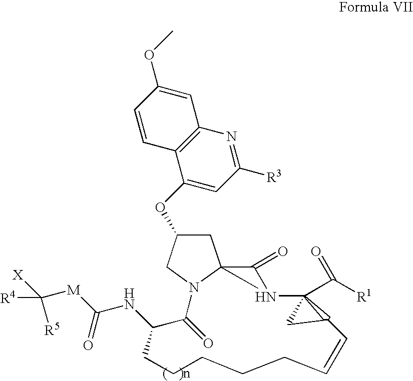

or a pharmaceutically acceptable salt, solvate or ester thereof, wherein,

M is O, N(H), or CH₂;

n is 0-4;

R¹ is —OR⁶, —NR⁶R⁷ or

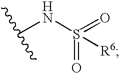

where R⁶ and R⁷ can be the same or different, each being independently selected from the group consisting of hydrogen, alkyl, alkenyl, alkynyl, heteroalkyl, cycloalkyl, cycloalkylalkyl, aryl, arylalkyl, heteroaryl, heteroarylalkyl, heterocyclyl, heterocyclylalkyl, hydroxyl, amino, arylamino and alkylamino;

R⁴ and R⁵ can be the same or different, each being independently selected from the group consisting of H, alkyl, aryl and cycloalkyl; or alternatively R⁴ and R⁵ together form part of a cyclic 5- to 7-membered ring such that the moiety 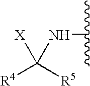
is represented by

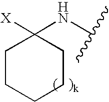

where k is 0 to 2;

X is selected from the group consisting of:

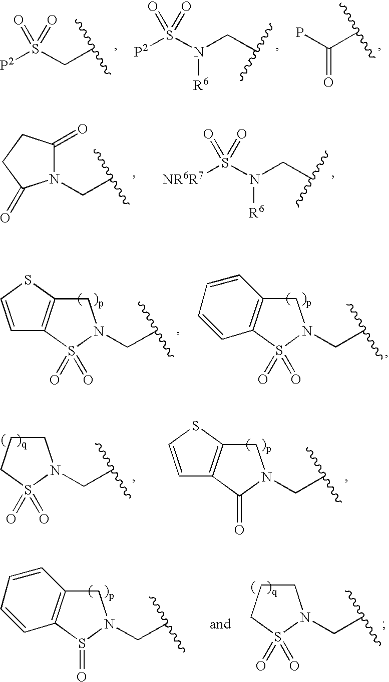

where p is 1 to 2, q is 1-3 and p² is alkyl, aryl, heteroaryl, heteroalkyl, cycloalkyl, dialkylamino, alkylamino, arylamino or cycloalkylamino; and

R³ is selected from the group consisting of: aryl, heterocyclyl, heteroaryl,

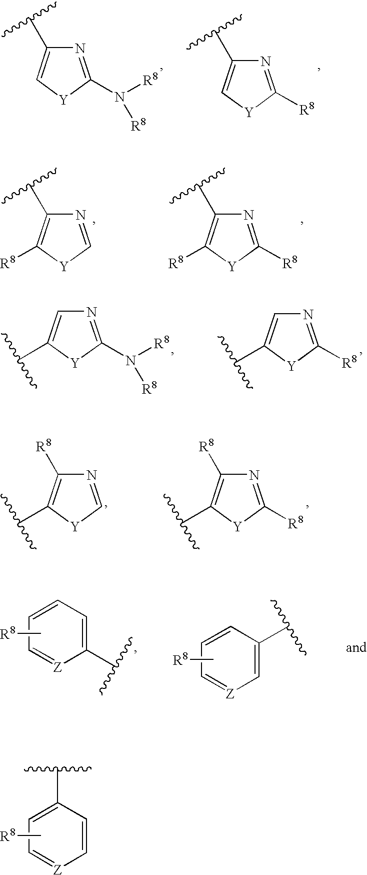

where Y is O, S or NH, and Z is CH or N, and the R⁸ moieties can be the same or different, each R⁸ being independently selected from the group consisting of hydrogen, alkyl, heteroalkyl, cycloalkyl, aryl, heteroaryl, heterocyclyl, hydroxyl, amino, arylamino, alkylamino, dialkylamino, halo, alkylthio, arylthio and alkyloxy.

In another embodiment, the compound is a compound of formula formula VIII:

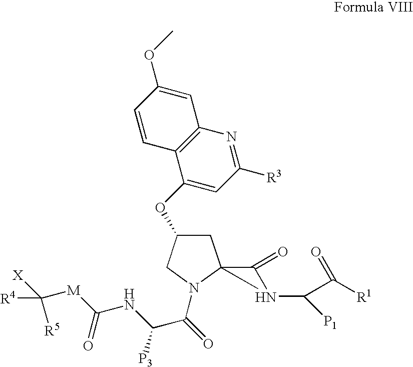

or a pharmaceutically acceptable salt, solvate or ester thereof, wherein,

M is O, N(H), or CH₂;

R¹ is —OR⁶, —NR⁶R⁷ or

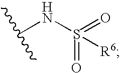

where R⁶ and R⁷ can be the same or different, each being independently selected from the group consisting of hydrogen, alkyl, alkenyl, alkynyl, heteroalkyl, cycloalkyl, cycloalkylalkyl, aryl, arylalkyl, heteroaryl, heteroarylalkyl, heterocyclyl, heterocyclylalkyl, hydroxyl, amino, arylamino and alkylamino;

P₁ is selected from the group consisting of alkyl, alkenyl, alkynyl, cycloalkyl haloalkyl;

P₃ is selected from the group consisting of alkyl, cycloalkyl, aryl and cycloalkyl fused with aryl;

R⁴ and R⁵ can be the same or different, each being independently selected from the group consisting of H, alkyl, aryl and cycloalkyl; or alternatively R⁴ and R⁵ together form part of a cyclic 5- to 7-membered ring such that the moiety 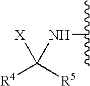
is represented by

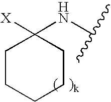

where k is 0 to 2;

X is selected from the group consisting of:

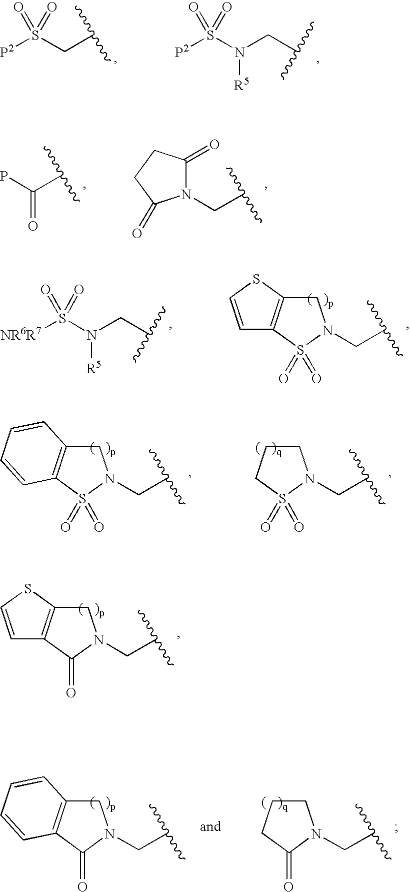

where p is 1 to 2, q is 1 to 3 and P² is alkyl, aryl, heteroaryl, heteroalkyl, cycloalkyl, dialkylamino, alkylamino, arylamino or cycloalkylamino; and

R³ is selected from the group consisting of: aryl, heterocyclyl, heteroaryl,

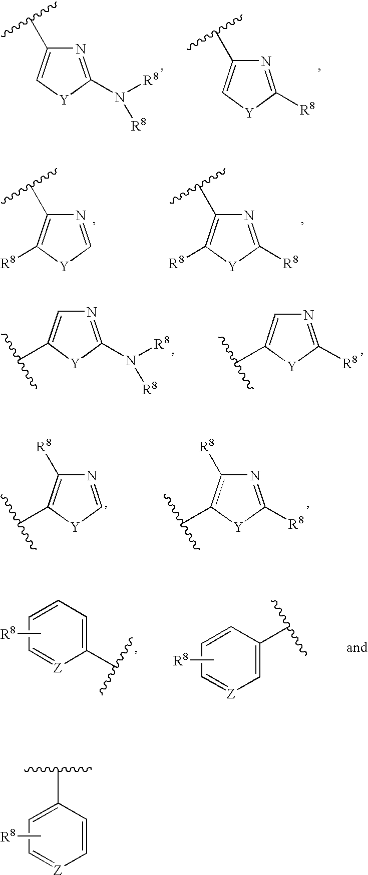

where Y is O, S or NH, and Z is CH or N, and the R⁸ moieties can be the same or different, each R⁸ being independently selected from the group consisting of hydrogen, alkyl, heteroalkyl, cycloalkyl, aryl, heteroaryl, heterocyclyl, hydroxyl, amino, arylamino, alkylamino, dialkylamino, halo, alkylthio, arylthio and alkyloxy.

In another embodiment, the compound is a compound of formula formula IX:

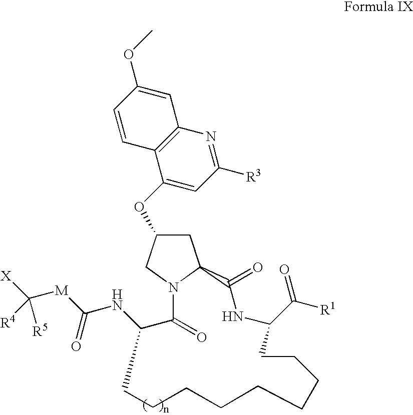

or a pharmaceutically acceptable salt, solvate or ester thereof, wherein,

M is O, N(H), or CH₂;

n is 0-4;

R¹ is —OR⁶, —NR⁶R⁷ or

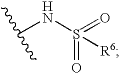

where R⁶ and R⁷ can be the same or different, each being independently selected from the group consisting of hydrogen, alkyl, alkenyl, alkynyl, heteroalkyl, cycloalkyl, cycloalkylalkyl, aryl, arylalkyl, heteroaryl, heteroarylalkyl, heterocyclyl, heterocyclylalkyl, hydroxyl, amino, arylamino and alkylamino;

R⁴ and R⁵ can be the same or different, each being independently selected from the group consisting of H, alkyl, aryl and cycloalkyl; or alternatively R⁴ and R⁵ together form part of a cyclic 5- to 7-membered ring such that the moiety 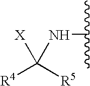
is represented by

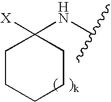

where k is 0 to 2;

X is selected from the group consisting of:

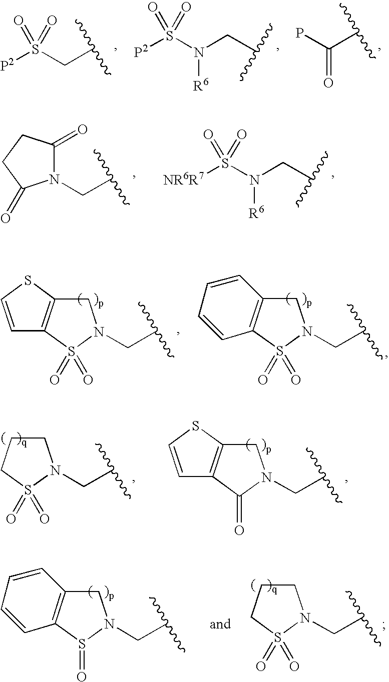

where p is 1 to 2, q is 1 to 3 and P² is alkyl, aryl, heteroaryl, heteroalkyl, cycloalkyl, dialkylamino, alkylamino, arylamino or cycloalkylamino; and

R³ is selected from the group consisting of: aryl, heterocyclyl, heteroaryl,

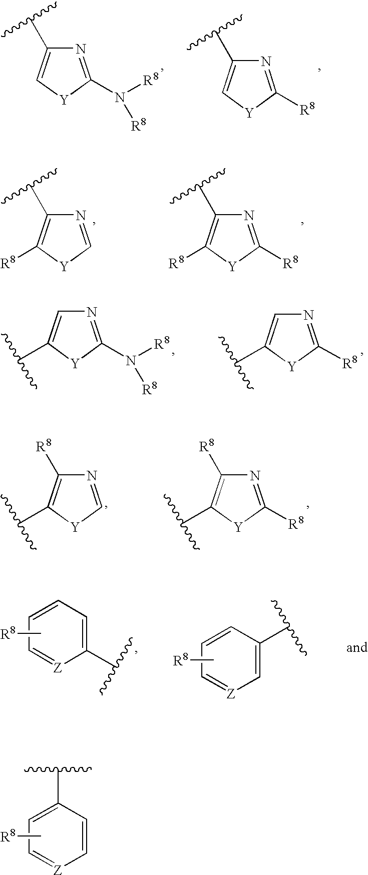

where Y is O, S or NH, and Z is CH or N, and the R⁸ moieties can be the same or different, each R⁸ being independently selected from the group consisting of hydrogen, alkyl, heteroalkyl, cycloalkyl, aryl, heteroaryl, heterocyclyl, hydroxyl, amino, arylamino, alkylamino, dialkylamino, halo, alkylthio, arylthio and alkyloxy.

In another embodiment, the compound is a compound of formula formula X:

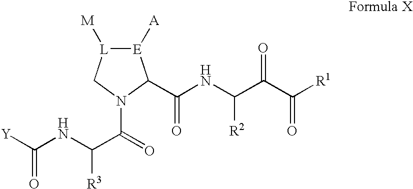

or a pharmaceutically acceptable salt, solvate or ester thereof; wherein:

R¹ is H, OR⁸, NR⁹R¹⁰, or CHR⁹R¹⁰, wherein R⁸, R⁹ and R₁₀ can be the same or different, each being independently selected from the group consisting of H, alkyl-, alkenyl-, alkynyl-, aryl-, heteroalkyl-, heteroaryl-, cycloalkyl-, heterocyclyl-, arylalkyl-, and heteroarylalkyl;

A and M can be the same or different, each being independently selected from R, OR, NHR, NRR′, SR, SO₂R, and halo; or A and M are connected to each other such that the moiety:

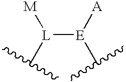

shown above in Formula I forms either a three, four, six, seven or eight-membered cycloalkyl, a four to eight-membered heterocyclyl, a six to ten-membered aryl, or a five to ten-membered heteroaryl;

E is C(H) or C(R);

L is C(H), C(R), CH₂C(R), or C(R)CH₂;

R, R′, R², and R³ can be the same or different, each being independently selected from the group consisting of H, alkyl-, alkenyl-, alkynyl-, cycloalkyl-, heteroalkyl-, heterocyclyl-, aryl-, heteroaryl-, (cycloalkyl)alkyl-, (heterocyclyl)alkyl-, aryl-alkyl-, and heteroaryl-alkyl-; or alternately R and R′ in NRR′ are connected to each other such that NRR′ forms a four to eight-membered heterocyclyl;

and Y is selected from the following moieties:

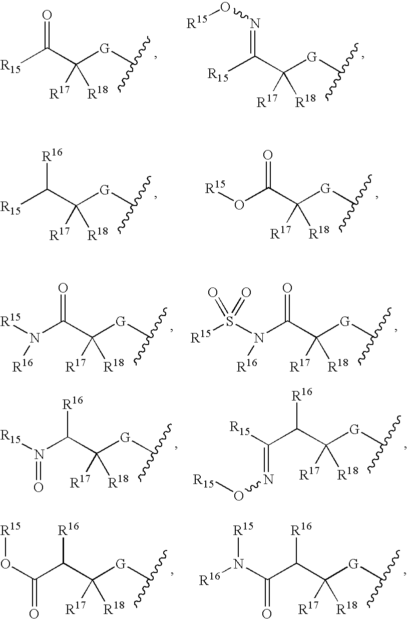

wherein G is NH or O; and R¹⁵, R¹⁶, R¹⁷ and R¹⁸ can be the same or different, each being independently selected from the group consisting of H, alkyl, heteroalkyl, alkenyl, heteroalkenyl, alkynyl, heteroalkynyl, cycloalkyl, heterocyclyl, aryl, arylalkyl, heteroaryl, and heteroarylalkyl, or alternately, R¹⁵ and R¹⁶ are connected to each other to form a four to eight-membered cycloalkyl, heteroaryl or heterocyclyl structure, and likewise, independently R¹⁷ and R¹⁸ are connected to each other to form a three to eight-membered cycloalkyl or heterocyclyl;

wherein each of said alkyl, aryl, heteroaryl, cycloalkyl or heterocyclyl can be unsubstituted or optionally independently substituted with one or more moieties selected from the group consisting of: hydroxy, alkoxy, aryloxy, thio, alkylthio, arylthio, amino, amido, alkylamino, arylamino, alkylsulfonyl, arylsulfonyl, sulfonamido, alkyl, aryl, heteroaryl, alkylsulfonamido, arylsulfonamido, keto, carboxy, carbalkoxy, carboxamido, alkoxycarbonylamino, alkoxycarbonyloxy, alkylureido, arylureido, halo, cyano, and nitro.

In one embodiment, the compound is a compound of Formula XI:

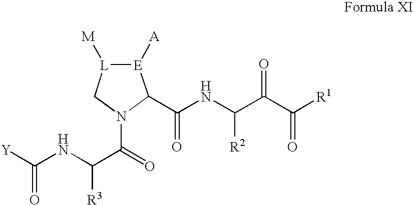

or a pharmaceutically acceptable salt, solvate or ester thereof; wherein:

R¹ is H, OR⁸, NR⁹R¹⁰, or CHR⁹R¹⁰, wherein R⁸, R⁹ and R¹⁰ can be the same or different, each being independently selected from the group consisting of H, alkyl-, alkenyl-, alkynyl-, aryl-, heteroalkyl-, heteroaryl-, cycloalkyl-, heterocyclyl-, arylalkyl-, and heteroarylalkyl;

A and M can be the same or different, each being independently selected from R,

NR⁹R¹⁰, SR, SO₂R, and halo; or A and M are connected to each other (in other words, A-E-L-M taken together) such that the moiety:

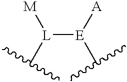

shown above in Formula I forms either a three, four, six, seven or eight-membered cycloalkyl, a four to eight-membered heterocyclyl, a six to ten-membered aryl, or a five to ten-membered heteroaryl;

E is C(H) or C(R);

L is C(H), C(R), CH₂C(R), or C(R)CH₂;

R, R′, R², and R³ can be the same or different, each being independently selected from the group consisting of H, alkyl-, alkenyl-, alkynyl-, cycloalkyl-, heteroalkyl-, heterocyclyl-, aryl-, heteroaryl-, (cycloalkyl)alkyl-, (heterocyclyl)alkyl-, aryl-alkyl-, and heteroaryl-alkyl-; or alternately R and R′ in NRR′ are connected to each other such that NR⁹R¹⁰ forms a four to eight-membered heterocyclyl;

Y is selected from the following moieties:

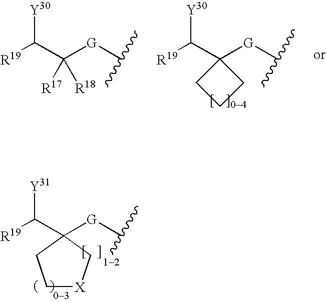

wherein Y³⁰ and Y³¹are selected from

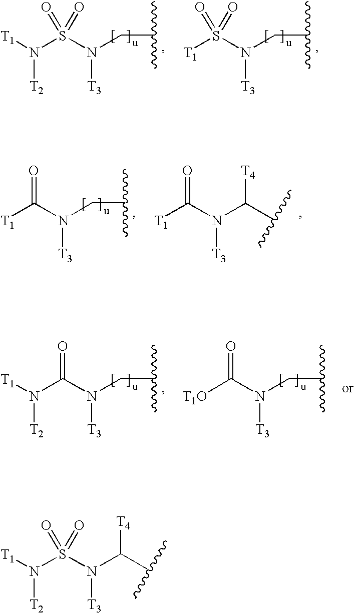

where u is a number 0-6;

X is selected from O, NR¹⁵, NC(O)R¹⁶, S, S(O) and SO₂;

G is NH or O; and

R¹⁵, R¹⁶, R¹⁷, R¹⁸, R¹⁹, T₁, T₂, T₃ and T₄ can be the same or different, each be independently selected from the group consisting of H, alkyl, heteroalkyl, alkenyl, heteroalkenyl, alkynyl, heteroalkynyl, cycloalkyl, heterocyclyl, aryl, arylalkyl, heteroaryl, and heteroarylalkyl, or alternately, R¹⁷ and R¹⁸ are connected to each other to form a three to eight-membered cycloalkyl or heterocyclyl;

wherein each of said alkyl, aryl, heteroaryl, cycloalkyl or heterocyclyl can be unsubstituted or optionally independently substituted with one or more moieties selected from the group consisting of: hydroxy, alkoxy, aryloxy, thio, alkylthio, arylthio, amino, amido, alkylamino, arylamino, alkylsulfonyl, arylsulfonyl, sulfonamido, alkyl, aryl, heteroaryl, alkylsulfonamido, arylsulfonamido, keto, carboxy, carbalkoxy, carboxamido, alkoxycarbonylamino, alkoxycarbonyloxy, alkylureido, arylureido, halo, cyano, and nitro.

In another embodiment, the compound is a compound of formula XII:

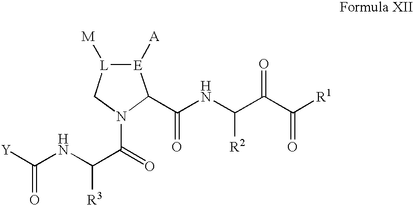

or a pharmaceutically acceptable salt, solvate or ester thereof; wherein:

R¹ is H, OR⁸, NR⁹R¹⁰, or CHR⁹R¹⁰, wherein R⁸, R⁹ and R¹⁰ can be the same or different, each being independently selected from the group consisting of H, alkyl-, alkenyl-, alkynyl-, aryl-, heteroalkyl-, heteroaryl-, cycloalkyl-, heterocyclyl-, arylalkyl-, and heteroarylalkyl;

A and M can be the same or different, each being independently selected from R, OR, NHR, NRR′, SR, SO₂R, and halo; or A and M are connected to each other such that the moiety:

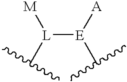

shown above in Formula I forms either a three, four, six, seven or eight-membered cycloalkyl, a four to eight-membered heterocyclyl, a six to ten-membered aryl, or a five to ten-membered heteroaryl;

E is C(H) or C(R);

L is C(H), C(R), CH₂C(R), or C(R)CH₂;

R, R′, R², and R³ can be the same or different, each being independently selected from the group consisting of H, alkyl-, alkenyl-, alkynyl-, cycloalkyl-, heteroalkyl-, heterocyclyl-, aryl-, heteroaryl-, (cycloalkyl)alkyl-, (heterocyclyl)alkyl-, aryl-alkyl-, and heteroaryl-alkyl-; or alternately R and R′ in NRR′ are connected to each other such that NRR′ forms a four to eight-membered heterocyclyl;

and Y is selected from the following moieties:

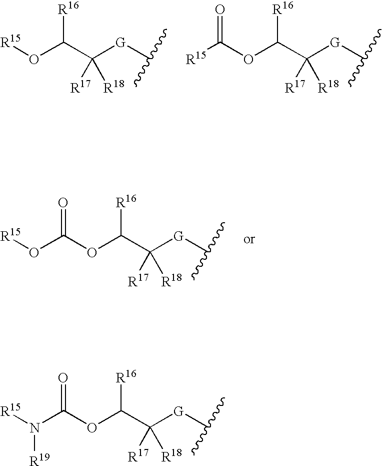

wherein G is NH or O; and R¹⁵, R¹⁶, R¹⁷, R¹⁸, and R¹⁹ can be the same or different, each being independently selected from the group consisting of H, alkyl, heteroalkyl, alkenyl, heteroalkenyl, alkynyl, heteroalkynyl, cycloalkyl, heterocyclyl, aryl, arylalkyl, heteroaryl, and heteroarylalkyl, or alternately, (i) either R¹⁵ and R¹⁶ are connected to each other to form a four to eight-membered cyclic structure, or R¹⁵ and R¹⁹ are connected to each other to form a four to eight-membered cyclic structure, and (ii) likewise, independently, R¹⁷ and R¹⁸ are connected to each other to form a three to eight-membered cycloalkyl or heterocyclyl;

wherein each of said alkyl, aryl, heteroaryl, cycloalkyl or heterocyclyl can be unsubstituted or optionally independently substituted with one or more moieties selected from the group consisting of: hydroxy, alkoxy, aryloxy, thio, alkylthio, arylthio, amino, amido, alkylamino, arylamino, alkylsulfonyl, arylsulfonyl, sulfonamido, alkylsulfonamido, arylsulfonamido, alkyl, aryl, heteroaryl, keto, carboxy, carbalkoxy, carboxamido, alkoxycarbonylamino, alkoxycarbonyloxy, alkylureido, arylureido, halo, cyano, and nitro.

In another embodiment, the compound is a compound of Formula XIII:

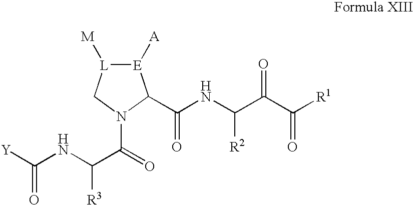

or a pharmaceutically acceptable salt, solvate or ester thereof; wherein:

R¹ is H, OR⁸, NR⁹R¹⁰, or CHR⁹R¹⁰, wherein R⁸, R⁹ and R¹⁰ can be the same or different, each being independently selected from the group consisting of H, alkyl-, alkenyl-, alkynyl-, aryl-, heteroalkyl-, heteroaryl-, cycloalkyl-, heterocyclyl-, arylalkyl-, and heteroarylalkyl;

A and M can be the same or different, each being independently selected from R, OR, NHR, NRR′, SR, SO₂R, and halo; or A and M are connected to each other (in other words, A-E-L-M taken together) such that the moiety:

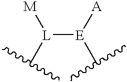

shown above in Formula I forms either a three, four, six, seven or eight-membered cycloalkyl, a four to eight-membered heterocyclyl, a six to ten-membered aryl, or a five to ten-membered heteroaryl;

E is C(H) or C(R);

L is C(H), C(R), CH₂C(R), or C(R)CH₂;

R, R′, R², and R³ can be the same or different, each being independently selected from the group consisting of H, alkyl-, alkenyl-, alkynyl-, cycloalkyl-, heteroalkyl-, heterocyclyl-, aryl-, heteroaryl-, (cycloalkyl)alkyl-, (heterocyclyl)alkyl-, aryl-alkyl-, and heteroaryl-alkyl-; or alternately R and R′ in NRR′ are connected to each other such that NRR′ forms a four to eight-membered heterocyclyl;

and Y is selected from the following moieties:

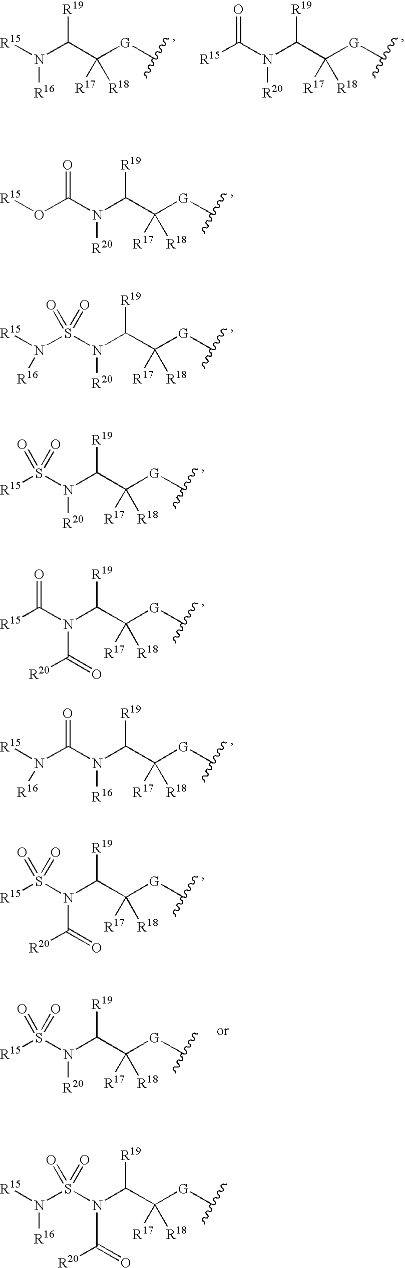

wherein G is NH or O, and R¹⁵, R^{16 l , R}¹⁷, R¹⁸, R¹⁹ and R²⁰ can be the same or different, each being independently selected from the group consisting of H, C₁-C₁₀ alkyl, C₁-C₁₀ heteroalkyl, C₂-C₁₀ alkenyl, C₂-C₁₀ heteroalkenyl, C₂-C₁₀ alkynyl, C₂-C₁₀ heteroalkynyl, C₃-C₈ cycloalkyl, C₃-C₈ heterocyclyl, aryl, heteroaryl, or alternately: (i) either R¹⁵ and R¹⁶ can be connected to each other to form a four to eight-membered cycloalkyl or heterocyclyl, or R¹⁵ and R¹⁹ are connected to each other to form a five to eight-membered cycloalkyl or heterocyclyl, or R¹⁵ and R²⁰ are connected to each other to form a five to eight-membered cycloalkyl or heterocyclyl, and (ii) likewise, independently, R¹⁷ and R¹⁸ are connected to each other to form a three to eight-membered cycloalkyl or heterocyclyl,

wherein each of said alkyl, aryl, heteroaryl, cycloalkyl or heterocyclyl can be unsubstituted or optionally independently substituted with one or more moieties selected from the group consisting of: hydroxy, alkoxy, aryloxy, thio, alkylthio, arylthio, amino, amido, alkylamino, arylamino, alkylsulfonyl, arylsulfonyl, sulfonamido, alkylsulfonamido, arylsulfonamido, keto, carboxy, carbalkoxy, carboxamido, alkoxycarbonylamino, alkoxycarbonyloxy, alkylureido, arylureido, halo, cyano, and nitro.

In another embodiment, the compound is a compound of Formula XIV:

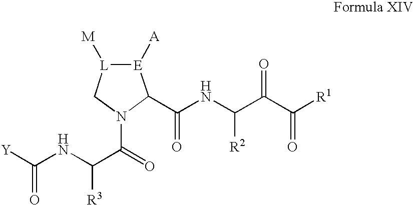

or a pharmaceutically acceptable salt, solvate or ester thereof; wherein:

R¹ is H, OR⁸, NR⁹R¹⁰, or CHR⁹R¹⁰, wherein R⁸, R⁹ and R₁₀ can be the same or different, each being independently selected from the group consisting of H, alkyl-, alkenyl-, alkynyl-, aryl-, heteroalkyl-, heteroaryl-, cycloalkyl-, heterocyclyl-, arylalkyl-, and heteroarylalkyl;

A and M can be the same or different, each being independently selected from R, OR, NHR, NRR′, SR, SO₂R, and halo;

or A and M are connected to each other such that the moiety:

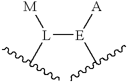

shown above in Formula I forms either a three, four, six, seven or eight-membered cycloalkyl, a four to eight-membered heterocyclyl, a six to ten-membered aryl, or a five to ten-membered heteroaryl;

E is C(H) or C(R);

L is C(H), C(R), CH₂C(R), or C(R)CH₂;

R, R′, R², and R³ can be the same or different, each being independently selected from the group consisting of H, alkyl, heteroalkyl, alkenyl, heteroalkenyl, alkynyl, heteroalkynyl, cycloalkyl, heterocyclyl, aryl, arylalkyl, heteroaryl, and heteroarylalkyl, or alternately R and R′ in NRR′ are connected to each other such that NRR′ forms a four to eight-membered heterocyclyl;

and Y is selected from the following moieties:

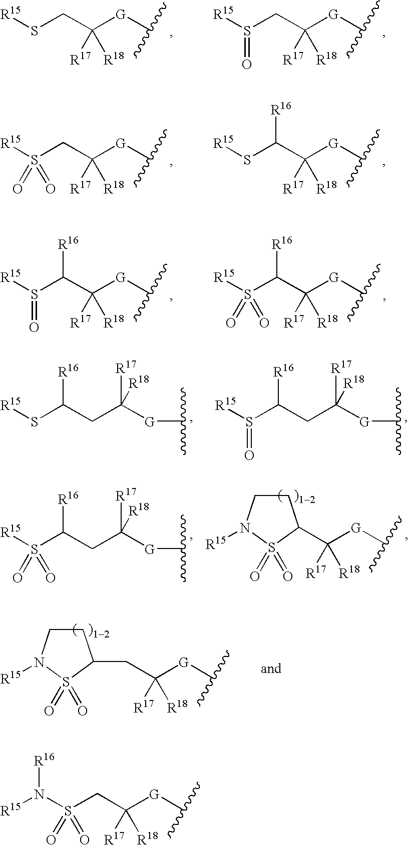

wherein G is NH or O; and R¹⁵, R¹⁶, R¹⁷ and R¹⁸ can be the same or different, each being independently selected from the group consisting of H, alkyl, heteroalkyl, alkenyl, heteroalkenyl, alkynyl, heteroalkynyl, cycloalkyl, heterocyclyl, aryl, and heteroaryl, or alternately, (i) R¹⁵ and R¹⁶ are connected to each other to form a four to eight-membered cyclic structure, and (ii) likewise., independently R¹⁷ and R¹⁸ are connected to each other to form a three to eight-membered cycloalkyl or heterocyclyl;

wherein each of said alkyl, aryl, heteroaryl, cycloalkyl or heterocyclyl can be unsubstituted or optionally independently substituted with one or more moieties selected from the group consisting of: hydroxy, alkoxy, aryloxy, thio, alkylthio, arylthio, amino, amido, alkylamino, arylamino, alkylsulfonyl, arylsulfonyl, sulfonamido, alkylsulfonamido, arylsulfonamido, alkyl, aryl, heteroaryl, keto, carboxy, carbalkoxy, carboxamido, alkoxycarbonylamino, alkoxycarbonyloxy, alkylureido, arylureido, halo, cyano, and nitro.

In another embodiment, the compound is a compound of formula Formula XV:

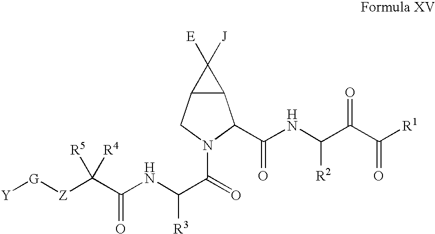

or a pharmaceutically acceptable salt, solvate or ester thereof; wherein:

R¹ is H, OR⁸, NR⁹R¹⁰, or CHR⁹R¹⁰, wherein R⁸, R⁹ and R¹⁰ can be the same or different, each being independently selected from the group consisting of H, alkyl-, aryl-, heteroalkyl-, heteroaryl-, cycloalkyl-, cycloalkyl-, arylalkyl-, and heteroarylalkyl; E and J can be the same or different, each being independently selected from the group consisting of R, OR, NHR, NRR⁷, SR, halo, and S(O₂)R, or E and J can be directly connected to each other to form either a three to eight-membered cycloalkyl, or a three to eight-membered heterocyclyl moiety;

Z is N(H), N®, or O, with the proviso that when Z is O, G is present or absent and if G is present with Z being O, then G is C(═O);

G maybe present or absent, and if G is present, G is C(═O) or S(O₂), and when G is absent, Z is directly connected to Y;

Y is selected from the group consisting of:

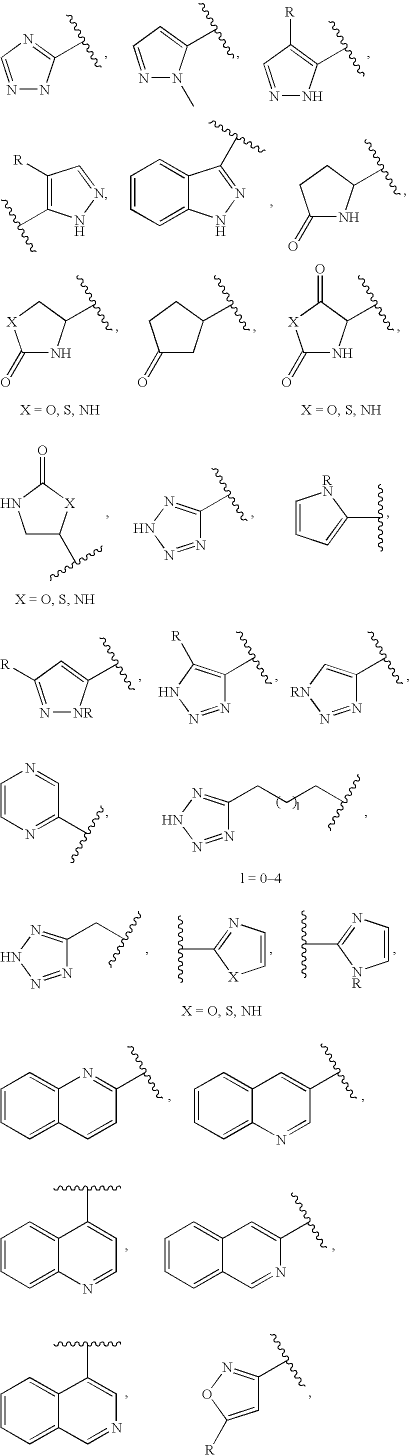

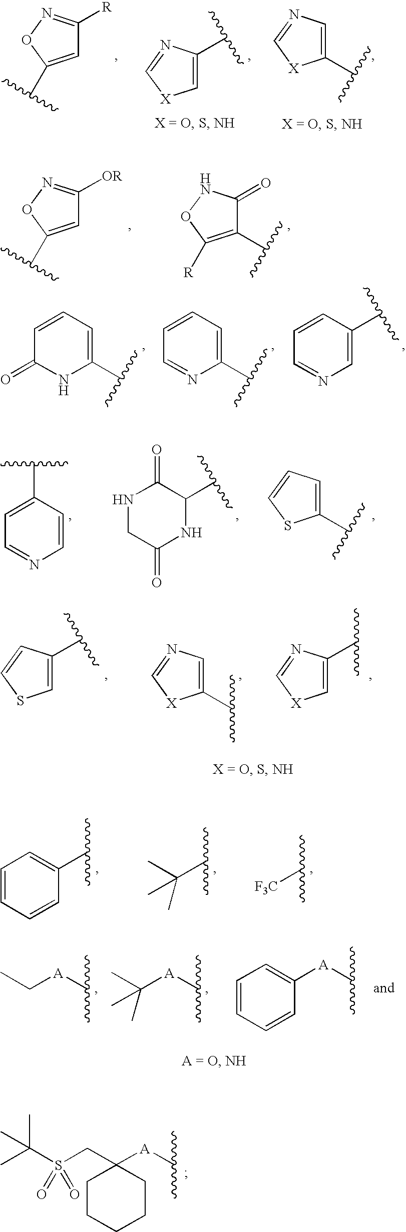

R, R⁷, R², R³, R⁴ and R⁵ can be the same or different, each being independently selected from the group consisting of H, alkyl-, alkenyl-, alkynyl-, cycloalkyl-, heteroalkyl-, heterocyclyl-, aryl-, heteroaryl-, (cycloalkyl)alkyl-, (heterocyclyl)alkyl-, aryl-alkyl-, and heteroaryl-alkyl-, wherein each of said heteroalkyl, heteroaryl and heterocyclyl independently has one to six oxygen, nitrogen, sulfur, or phosphorus atoms;

wherein each of said alkyl, heteroalkyl, alkenyl, alkynyl, aryl, heteroaryl, cycloalkyl and heterocyclyl moieties can be unsubstituted or optionally independently substituted with one or more moieties selected from the group consisting of alkyl, alkenyl, alkynyl, aryl, aralkyl, cycloalkyl, heterocyclyl, halo, hydroxy, thio, alkoxy, aryloxy, alkylthio, arylthio, amino, amido, ester, carboxylic acid, carbamate, urea, ketone, aldehyde, cyano, nitro, sulfonamido, sulfoxide, sulfone, sulfonyl urea, hydrazide, and hydroxamate.

In another embodiment, the compound is a compound of Formula XVI:

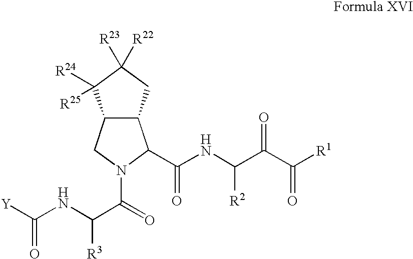

or a pharmaceutically acceptable salt, solvate or ester thereof; wherein:

R¹ is H, OR⁸, NR⁹R¹⁰, or CHR⁹R¹⁰, wherein R⁸, R⁹ and R¹⁰ can be the same or different, each being independently selected from the group consisting of H, alkyl-, alkenyl-, alkynyl-, aryl-, heteroalkyl-, heteroaryl-, cycloalkyl-, heterocyclyl-, arylalkyl-, and heteroarylalkyl, or alternately R⁹ and R¹⁰ in NR⁹R¹⁰ are connected to each other such that NR⁹R¹⁰ forms a four to eight-membered heterocyclyl, and likewise independently alternately R⁹ and R¹⁰ in CHR⁹R¹⁰ are connected to each other such that CHR⁹R¹⁰ forms a four to eight-membered cycloalkyl;

R² and R³ can be the same or different, each being independently selected from the group consisting of H, alkyl, heteroalkyl, alkenyl, heteroalkenyl, alkynyl, heteroalkynyl, cycloalkyl, heterocyclyl, aryl, arylalkyl, heteroaryl, and heteroarylalkyl;

Y is selected from the following moieties:

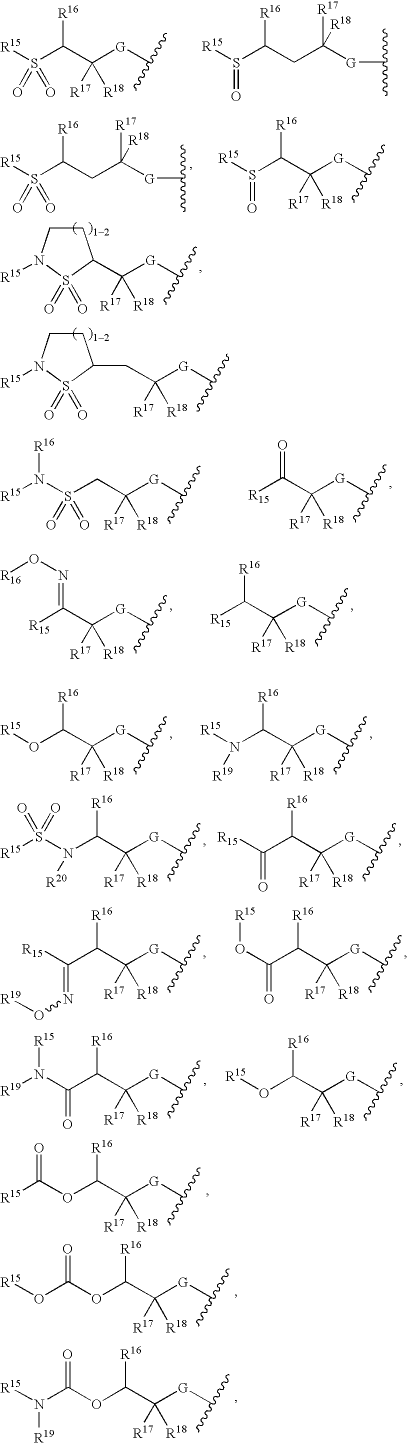

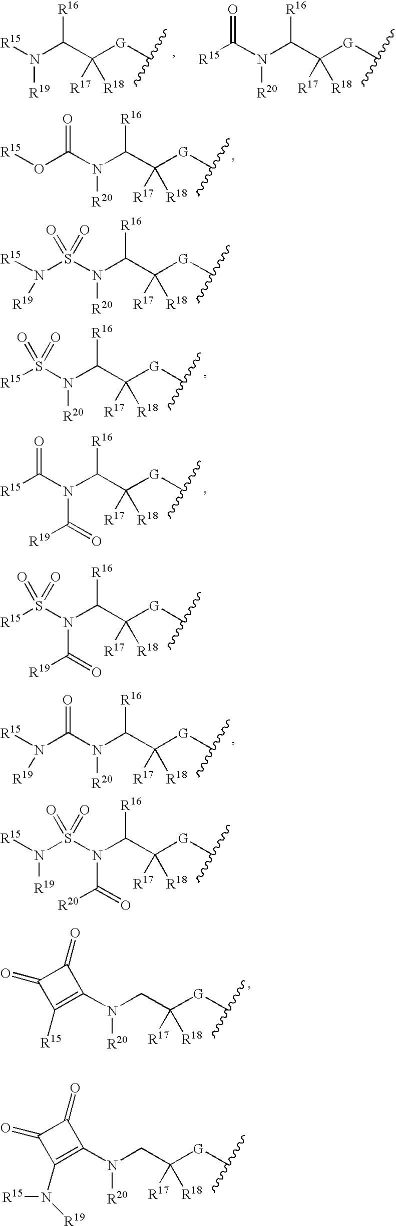

wherein G is NH or O; and R¹⁵, R¹⁶, R¹⁷, R¹⁸, R¹⁹, R²⁰ R²¹ R²¹, R²³, R²⁴ and R²⁵ can be the same or different, each being independently selected from the group consisting of H, alkyl, heteroalkyl, alkenyl, heteroalkenyl, alkynyl, heteroalkynyl, cycloalkyl, heterocyclyl, aryl, arylalkyl, heteroaryl, and heteroarylalkyl, or alternately (i) R¹⁷ and R¹⁸ are independently connected to each other to form a three to eight-membered cycloalkyl or heterocyclyl; (ii) likewise independently R¹⁵ and R¹⁹ are connected to each other to form a four to eight-membered heterocyclyl; (iii) likewise independently R¹⁵ and R¹⁶ are connected to each other to form a four to eight-membered heterocyclyl; (iv) likewise independently R¹⁵ and R²⁰ are connected to each other to form a four to eight-membered heterocyclyl; (v) likewise independently R²² and R²³ are connected to each other to form a three to eight-membered cycloalkyl or a four to eight-membered heterocyclyl; and (vi) likewise independently R²⁴ and R²⁵ are connected to each other to form a three to eight-membered cycloalkyl or a four to eight-membered heterocyclyl;

wherein each of said alkyl, aryl, heteroaryl, cycloalkyl or heterocyclyl can be unsubstituted or optionally independently substituted with one or more moieties selected from the group consisting of hydroxy, alkoxy, aryloxy, thio, alkylthio, arylthio, amino, amido, alkylamino, arylamino, alkylsulfonyl, arylsulfonyl, sulfonamido, alkyl, aryl, heteroaryl, alkylsulfonamido, arylsulfonamido, keto, carboxy, carbalkoxy, carboxamido, alkoxycarbonylamino, alkoxycarbonyloxy, alkylureido, arylureido, halo, cyano, and nitro.

In another embodiment, the compound is a compound of Formula XVII:

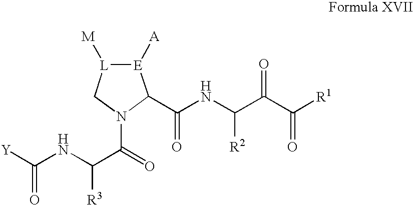

or a pharmaceutically acceptable salt, solvate or ester thereof; wherein:

R¹ is H, OR⁸, NR⁹R¹⁰, or CHR⁹R¹⁰, wherein R⁸, R⁹ and R¹⁰ can be the same or different, each being independently selected from the group consisting of H, alkyl-, alkenyl-, alkynyl-, aryl-, heteroalkyl-, heteroaryl-, cycloalkyl-, heterocyclyl-, arylalkyl-, and heteroarylalkyl;

A and M can be the same or different, each being independently selected from R, OR, NHR, NRR′, SR, SO₂R, and halo; or A and M are connected to each other such that the moiety:

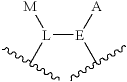

shown above in Formula I forms either a three, four, six, seven or eight-membered cycloalkyl, a four to eight-membered heterocyclyl, a six to ten-membered aryl, or a five to ten-membered heteroaryl;

E is C(H) or C®;

L is C(H), C®, CH₂C®, or COCH₂;

R, R′, R², and R³ can be the same or different, each being independently selected from the group consisting of H, alkyl-, alkenyl-, alkynyl-, cycloalkyl-, heteroalkyl-, heterocyclyl-, aryl-, heteroaryl-, (cycloalkyl)alkyl-, (heterocyclyl)alkyl-, aryl-alkyl-, and heteroaryl-alkyl-; or alternately R and R′ in NRR′ are connected to each other such that NRR′ forms a four to eight-membered heterocyclyl;

Y is selected from the following moieties:

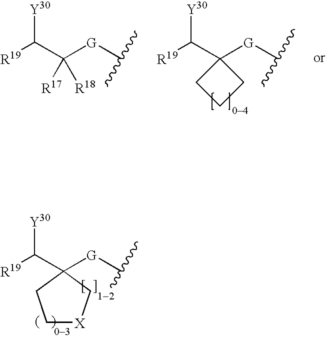

wherein Y³⁰ is selected from

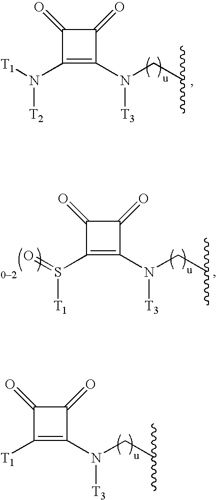

where u is a number 0-1;

X is selected from O, NR¹⁵, NC(O)R¹⁶, S, S(O) and SO₂;

G is NH or O; and

R¹⁵, R¹⁶, R¹⁷, R¹⁸, R¹⁹, T₁, T₂, and T₃ can be the same or different, each being independently selected from the group consisting of H, alkyl, heteroalkyl, alkenyl, heteroalkenyl, alkynyl, heteroalkynyl, cycloalkyl, heterocyclyl, aryl, arylalkyl, heteroaryl, and heteroarylalkyl, or alternately, R¹⁷ and R¹⁸ are connected to each other to form a three to eight-membered cycloalkyl or heterocyclyl;

wherein each of said alkyl, aryl, heteroaryl, cycloalkyl or heterocyclyl can be unsubstituted or optionally independently substituted with one or more moieties selected from the group consisting of: hydroxy, alkoxy, aryloxy, thio, alkylthio, arylthio, amino, amido, alkylamino, arylamino, alkylsulfonyl, arylsulfonyl, sulfonamido, alkyl, aryl, heteroaryl, alkylsulfonamido, arylsulfonamido, keto, carboxy, carbalkoxy, carboxamido, alkoxycarbonylamino, alkoxycarbonyloxy, alkylureido, arylureido, halo, cyano, and nitro.

In another embodiment, the compound is a compound of Formula XVIII:

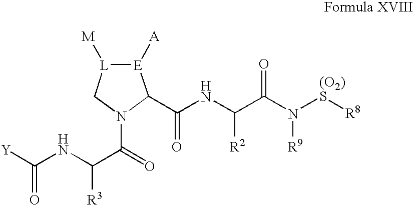

or a pharmaceutically acceptable salt, solvate or ester thereof, wherein:

R⁸ is selected from the group consisting of alkyl-, aryl-, heteroalkyl-, heteroaryl-, cycloalkyl-, heterocyclyl-, arylalkyl-, heteroarylalkyl- , and heterocyclylalkyl;

R⁹ is selected from the group consisting of H, alkyl, alkenyl, alkynyl, aryl and cycloalkyl;

A and M can be the same or different, each being independently selected from R, OR, N(H)R, N(RR′), SR, S(O₂)R, and halo; or A and M are connected to each other (in other words, A-E-L-M taken together) such that the moiety:

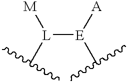

shown above in Formula I forms either a three, four, five, six, seven or eight-membered cycloalkyl, a four to eight-membered heterocyclyl, a six to ten-membered aryl, or a five to ten-membered heteroaryl;

E is C(H) or C®;

L is C(H), C®, CH₂C®, or C®CH₂;

R and R′ can be the same or different, each being independently selected from the group consisting of H, alkyl-, alkenyl-, alkynyl-, cycloalkyl-, heteroalkyl-, heterocyclyl-, aryl-, heteroaryl-, (cycloalkyl)alkyl-, (heterocyclyl)alkyl-, aryl-alkyl-, and heteroaryl-alkyl-; or alternately R and R′ in N(RR′) are connected to each other such that N(RR′) forms a four to eight-membered heterocyclyl;

R² and R³ can be the same or different, each being independently selected from the group consisting of H, alkyl, heteroalkyl, alkenyl, heteroalkenyl, alkynyl, heteroalkynyl, cycloalkyl, spiro-linked cycloalkyl, heterocyclyl, aryl, arylalkyl, heteroaryl, and heteroarylalkyl;

Y is selected from the following moieties:

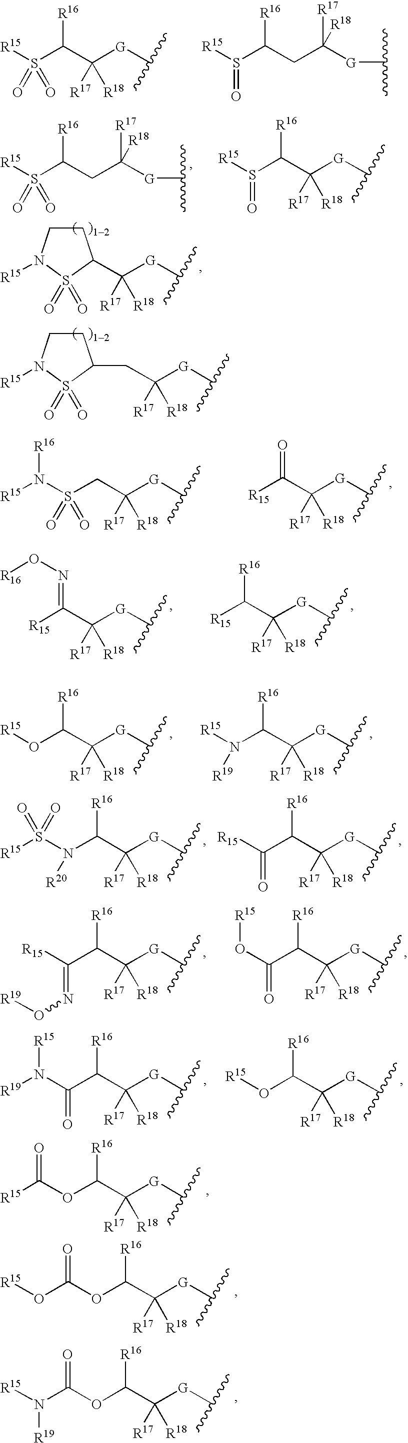

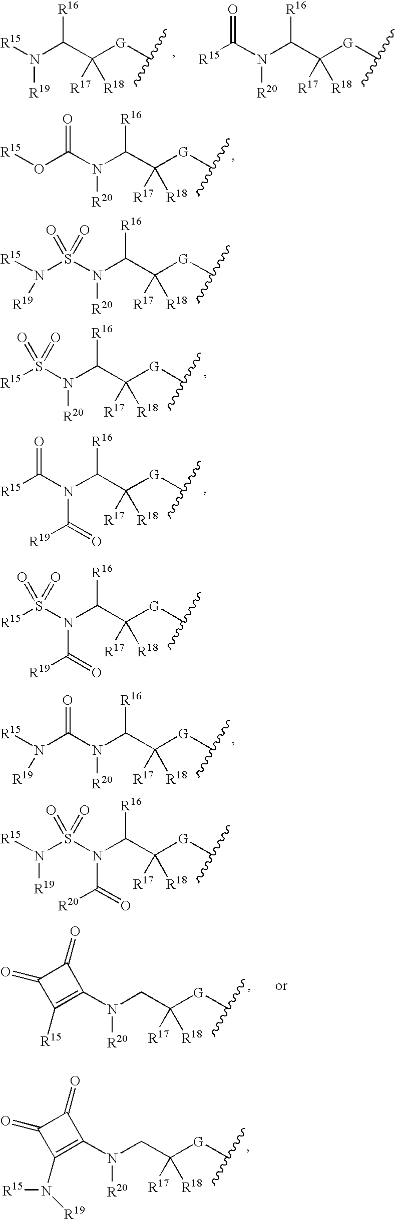

wherein G is NH or O; and R¹⁵, R¹⁶, R¹⁷, R¹⁸, R¹⁹ and R²⁰ can be the same or different, each being independently selected from the group consisting of H, alkyl, heteroalkyl, alkenyl, heteroalkenyl, alkynyl, heteroalkynyl, cycloalkyl, heterocyclyl, aryl, arylalkyl, heteroaryl, and heteroarylalkyl, or alternately (i) R¹⁷ and R¹⁸ and independently connected to each other to form a three to eight-membered cycloalkyl or heterocyclyl; (ii) likewise independently R¹⁵ and R¹⁹ are connected to each other to form a four or eight-membered heterocyclyl; (ii) likewise independently R¹⁵ and R¹⁶ are connected to each other to form a four to eight-membered heterocyclyl; and (iv) likewise independently R¹⁵ and R²⁰ are connected to each other to form a four to eight-membered heterocyclyl;

wherein each of said alkyl, aryl, heteroaryl, cycloalkyl, spiro-linked cycloalkyl, and heterocyclyl can be unsubstituted or optionally independently substituted with one or more moieties selected from the group consisting of hydroxy, alkoxy, aryloxy, thio, alkylthio, arylthio, amino, amido, alkylamino, arylamino, alkylsulfonyl, arylsulfonyl, sulfonamido, alkyl, alkenyl, aryl, heteroaryl, alkylsulfonamido, arylsulfonamido, keto, carboxy, carbalkoxy, carboxamido, alkoxycarbonylamino, alkoxycarbonyloxy, alkylureido, arylureido, halo, cyano, and nitro.

In another embodiment, the compound is a compound of Formula XIX:

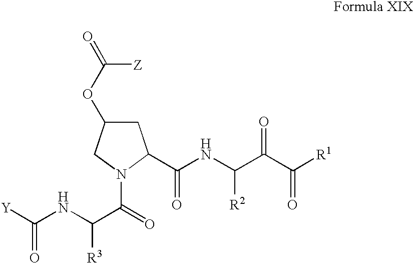

wherein;

Z is selected from the group consisting of a heterocyclyl moiety,

N(H)(alkyl), —N(alkyl)₂, —N(H)(cycloalkyl), —N(cycloalkyl)₂, —N(H)(aryl, —N(aryl)₂, —N(H)(heterocyclyl), —N(heterocyclyl)₂, —N(H)(heteroaryl), and —N(heteroaryl)₂;

R¹ is H, OR⁸, NR⁹R¹⁰, or CHR⁹R¹⁰, wherein R⁸, R⁹ and R¹⁰ can be the same or different, each being independently selected from the group consisting of H, alkyl-, alkenyl-, alkynyl-, aryl-, heteroalkyl-, heteroaryl-, cycloalkyl-, heterocyclyl-, arylalkyl-, and heteroarylalkyl, or alternately R⁹ and R¹⁰ in NR⁹R¹⁰ are connected to each other such that NR⁹R¹⁰ forms a four to eight-membered heterocyclyl, and likewise independently alternately R⁹ and R¹⁰ in CHR⁹R¹⁰ are connected to each other such that CHR⁹R¹⁰ forms a four to eight-membered cycloalkyl;

R² and R³ can be the same or different, each being independently selected from the group consisting of H, alkyl, heteroalkyl, alkenyl, heteroalkenyl, alkynyl, heteroalkynyl, cycloalkyl, heterocyclyl, aryl, arylalkyl, heteroaryl, and heteroarylalkyl;

Y is selected from the following moieties:

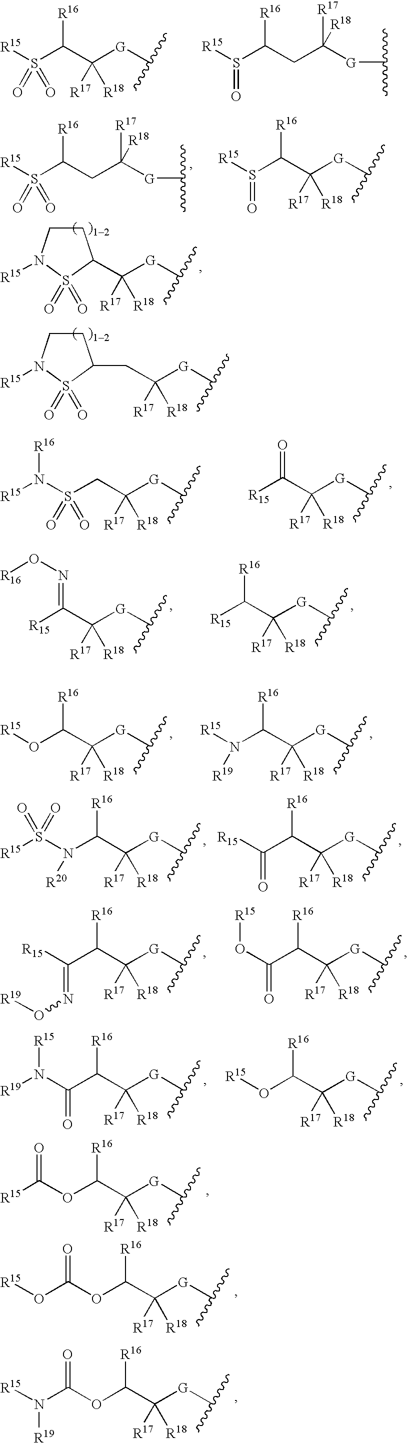

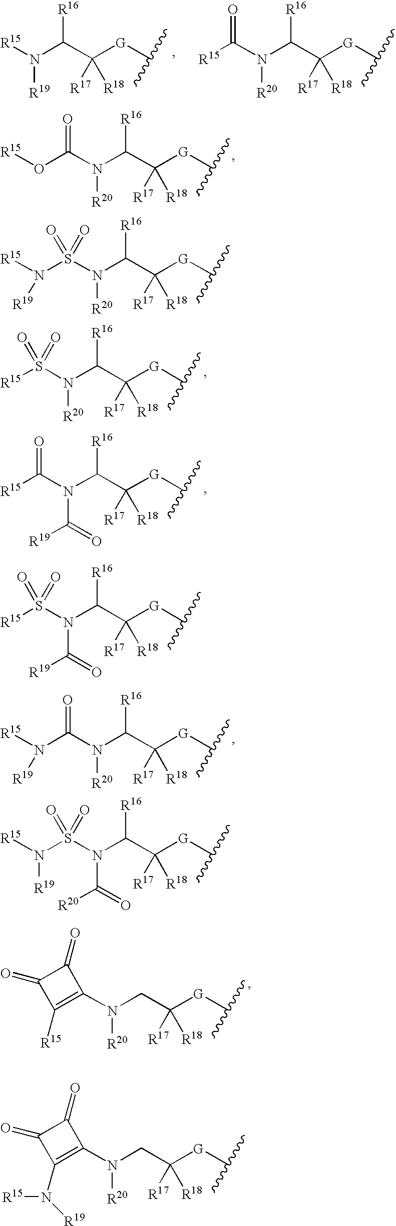

wherein G is NH or O; and R¹⁵, R¹⁶, R¹⁷, R¹⁸, R¹⁹, R²⁰and R²¹ can be the same or different, each being independently selected from the group consisting of H, alkyl, heteroalkyl, alkenyl, heteroalkenyl, alkynyl, heteroalkynyl, cycloalkyl, heterocyclyl, aryl, arylalkyl, heteroaryl, and heteroarylalkyl, or alternately (i) R¹⁷ and R¹⁸ are independently connected to each other to form a three to eight-membered cycloalkyl or heterocyclyl; (ii) likewise independently R¹⁵ and R¹⁹ are connected to each other to form a four to eight-membered heterocyclyl; (iii) likewise independently R¹⁵ and R¹⁶ are connected to each other to form a four to eight-membered heterocyclyl; and (iv) likewise independently R¹⁵ and R²⁰ are connected to each other to form a four to eight-membered heterocyclyl;

wherein each of said alkyl, aryl, heteroaryl, cycloalkyl or heterocyclyl can be unsubstituted or optionally independently substituted with one or more moieties selected from the group consisting of hydroxy, alkoxy, aryloxy, thio, alkylthio, arylthio, amino, amido, alkylamino, arylamino, alkylsulfonyl, arylsulfonyl, sulfonamido, alkyl, aryl, heteroaryl, alkylsulfonamido, arylsulfonamido, keto, carboxy, carbalkoxy, carboxamido, alkoxycarbonylamino, alkoxycarbonyloxy, alkylureido, arylureido, halo, cyano, and nitro.

In another embodiment, the compound is a compound of formula XX

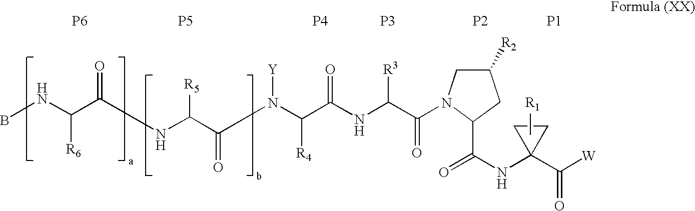

or a pharmaceutically acceptable salt, solvate or ester thereof; wherein: a is 0 or 1; b is 0 or 1; Y is H or C₁₋₆alkyl;

B is H, an acyl derivative of formula R₇—C(O)— or a sulfonyl of formula R₇—SO2 wherein

R7 is (i) C₁₋₁₀ alkyl optionally substituted with carboxyl, C₁₋₆ alkanoyloxy or C₁₋₆ alkoxy;
(ii) C₃₋₇ cycloalkyl optionally substituted with carboxyl, (C₁₋₆ alkoxy)carbonyl or phenylmethoxycarbonyl;
(iii) C₆ or C₁₀ aryl or C₇₋₁₆ aralkyl optionally substituted with C₁₋₆ alkyl, hydroxy, or amino optionally substituted with C₁₋₆ alkyl; or
(iv) Het optionally substituted with C₁₋₆ alkyl, hydroxy, amino optionally substituted with C₁₋₆ alkyl, or amido optionally substituted with C₁₋₆ alkyl;

R₆, when present, is C₁₋₆ alkyl substituted with carboxyl;

R₅, when present, is C₁₋₆ alkyl optionally substituted with carboxyl;

R₄ is C₁₋₁₀ alkyl, C₃₋₇ cycloalkyl or C₄₋₁₀ (alkylcycloalkyl);

R₃ is C₁₋₁₀ alkyl, C₃₋₇ cycloalkyl or C₄₋₁₀ (alkylcycloalkyl);

R₂ is CH₂—R₂₀, NH—R₂₀, 0-R₂₀ or S—R₂₀, wherein R₂₀ is a saturated or unsaturated C₃₋₇ cycloalkyl or C₄₋₁₀ (alkyl cycloalkyl) being optionally mono-, di- or tri-substituted with R₂₁, or R₂₀ is a C₆ or C₁₀ aryl or C₇₋₁₆ aralkyl optionally mono-, di- or tri-substituted with R₂₁,

or R₂₀ is Het or (lower alkyl)-Het optionally mono-, di- or tri-substituted with R₂₁, wherein each R₂₁ is independently C₁₋₆ alkyl; C₁₋₆alkoxy; amino optionally mono- or di-substituted with C₁₋₆ alkyl; sulfonyl; N0₂; OH; SH; halo; haloalkyl; amido optionally mono-substituted with C₁₋₆ alkyl, C₆ or C₁₀ aryl, C₇₋₁₆ aralkyl, Het or (lower alkyl)-Het; carboxyl; carboxy(lower alkyl); C₆ or C₁₀ aryl, C₇₋₁₆ aralkyl or Het, said aryl, aralkyl or Het being optionally substituted with R₂₂;

wherein R₂₂ is C₁₋₆alkyl; C₁₋₆ alkoxy; amino optionally mono- or di-substituted with C₁₋₆ alkyl; sulfonyl; N0₂; OH; SH; halo; haloalkyl; carboxyl; amide or (lower alkyl)amide;

R₁ is C₁₋₆ alkyl or C₂₋₆ alkenyl optionally substituted with halogen; and

W is hydroxy or a N-substituted amino.

In another embodiment, the compound is a compound of formula XXI

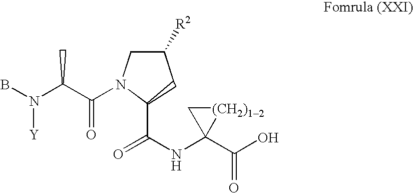

or a pharmaceutically acceptable salt, solvate or ester thereof; wherein:

B is H, a C₆ or C₁₀ aryl, C₇₋₁₆ aralkyl; Het or (lower alkyl)-Het, all of which optionally substituted with C₁₋₆ alkyl; C₁₋₆ alkoxy; C₁₋₆ alkanoyl; hydroxy; hydroxyalkyl; halo; haloalkyl; nitro; cyano; cyanoalkyl; amino optionally substituted with C₁₋₆ alkyl; amido; or (lower alkyl)amide;

or B is an acyl derivative of formula R₄—C(O)—; a carboxyl of formula R₄-0-C(O)—; an amide of formula R₄—N(R₅)—C(O)—; a thioamide of formula R₄—N(R₅)—C(S)—; or a sulfonyl of formula R₄—SO2 wherein

R₄ is (i) C₁₋₁₀ alkyl optionally substituted with carboxyl, C₁₋₆ alkanoyl, hydroxy, C₁₋₆ alkoxy, amino optionally mono- or di-substituted with C₁₋₆ alkyl, amido, or (lower alkyl) amide;
(ii) C₃₋₇ cycloalkyl, C₃₋₇ cycloalkoxy, or C₄₋₁₀ alkylcycloalkyl, all optionally substituted with hydroxy, carboxyl, (C₁₋₆ alkoxy)carbonyl, amino optionally mono- or di-substituted with C₁₋₆ alkyl, amido, or (lower alkyl) amide;
(iii) amino optionally mono- or di-substituted with C₁₋₆ alkyl; amido; or (lower alkyl)amide;
(iv) C₆ or C₁₀ aryl or C₇₋₁₆ aralkyl, all optionally substituted with C₁₋₆ alkyl, hydroxy, amido, (lower alkyl)amide, or amino optionally mono- or di-substituted with C₁₋₆ alkyl; or
(v) Het or (lower alkyl)-Het, both optionally substituted with C₁₋₆ alkyl, hydroxy, amido, (lower alkyl) amide, or amino optionally mono- or di-substituted with C₁₋₆ alkyl;

R₅ is H or C₁₋₆ alkyl;

with the proviso that when R₄ is an amide or a thioamide, R₄ is not (ii) a cycloalkoxy;

Y is H or C₁₋₆ alkyl;

R₃ is C₁₋₈ alkyl, C₃₋₇ cycloalkyl, or C₄₋₁₀ alkylcycloalkyl, all optionally substituted with hydroxy, C₁₋₆ alkoxy, C₁₋₆ thioalkyl, amido, (lower alkyl)amido, C₆ or C₁₀ aryl, or C₇₋₁₆ aralkyl;

R₂ is CH₂—R₂₀, NH—R₂₀, O—R₂₀ or S—R₂₀, wherein R₂₀ is a saturated or unsaturated C₃₋₇ cycloalkyl or C₄₋₁₀ (alkylcycloalkyl), all of which being optionally mono-, di- or tri-substituted with R₂₁, or R₂₀ is a C₆ or C₁₀ aryl or C₇₋₁₄ aralkyl, all optionally mono-, di- or tri-substituted with R₂₁,

or R₂₀ is Het or (lower alkyl)-Het, both optionally mono-, di- or tri-substituted with R₂₁,

wherein each R₂₁ is independently C₁₋₆ alkyl; C₁₋₆ alkoxy; lower thioalkyl; sulfonyl; N0₂; OH; SH; halo; haloalkyl; amino optionally mono- or di-substituted with C₁₋₆ alkyl, C₆ or C₁₀ aryl, C₇₋₁₄ aralkyl, Het or (lower alkyl)-Het; amido optionally mono-substituted with C₁₋₆ alkyl, C₆ or C₁₀ aryl, C₇₋₁₄ aralkyl, Het or (lower alkyl)-Het; carboxyl; carboxy(lower alkyl); C₆ or C₁₀ aryl, C₇₋₁₄ aralkyl or Het, said aryl, aralkyl or Het being optionally substituted with R₂₂;

wherein R₂₂ is C₁₋₆ alkyl; C₃₋₇ cycloalkyl; C₁₋₆ alkoxy; amino optionally mono- or di-substituted with C₁₋₆ alkyl; sulfonyl; (lower alkyl)sulfonyl; N0₂; OH; SH; halo; haloalkyl; carboxyl; amide; (lower alkyl)amide; or Het optionally substituted with C₁₋₆ alkyl;

R1 is H; C₁₋₆ alkyl, C₃₋₇ cycloalkyl, C₂₋₆ alkenyl, or C₂₋₆ alkynyl, all optionally substituted with halogen.

In another embodiment, the compound is a compound of formula XXII

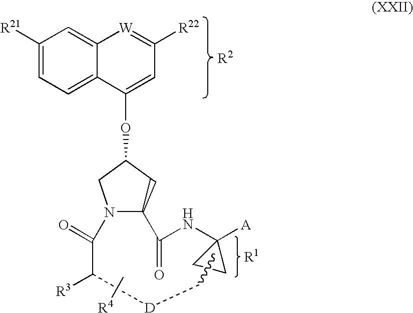

or a pharmaceutically acceptable salt, solvate or ester thereof; wherein

W is CH or N,

R²¹ is H, halo, C₁₋₆ alkyl, C₃₋₆ cycloalkyl, C₁₋₆ haloalkyl, C₁₋₆ alkoxy, C₃₋₆ cycloalkoxy, hydroxy, or N(R²³)₂ , wherein each R²³ is independently H, C₁₋₆ alkyl or C₃₋₆ cycloalkyl;

R²² is H, halo, C₁₋₆ alkyl, C₃₋₆ cycloalkyl, C₁₋₆ haloalkyl, C₁₋₆ thioalkyl, C₁₋₆ alkoxy, C₃₋₆ cycloalkoxy, C₂₋₇ alkoxyalkyl, C₃₋₆ cycloalkyl, C_{6 or 10} aryl or Het, wherein Het is a five-, six-, or seven-membered saturated or unsaturated heterocycle containing from one to four heteroatoms selected from nitrogen, oxygen and sulfur;

said cycloalkyl, aryl or Het being substituted with R²⁴, wherein R²⁴ is H, halo, C₁₋₆ alkyl, C₃₋₆ cycloalkyl, C₁₋₆ alkoxy, C₃₋₆ cycloalkoxy, NO₂ , N(R²⁵)₂ , NH—C(O)—R²⁵ or NH—C(O)—NH—R²⁵, wherein each R²⁵ is independently: H, C₁₋₆ alkyl or C₃₋₆ cycloalkyl;

or R²⁴ is NH—C(O)—OR²⁶ wherein R²⁶ is C₁₋₆ alkyl or C₃₋₆ cycloalkyl;

R³ is hydroxy, NH₂ , or a group of formula —NH—R³¹, wherein R³¹ is C_{6 or} 10 aryl, heteroaryl, —C(O)—R³², —C(O)—NHR³² or —C(O)—OR³², wherein R³² is C₁₋₆ alkyl or C₃₋₆ cycloalkyl;

D is a 5 to 1 0-atom saturated or unsaturated alkylene chain optionally containing one to three heteroatoms independently selected from: O, S, or N—R⁴¹ , wherein R⁴¹ is H, C₁₋₆ alkyl, C₃₋₆ cycloalkyl or —C(O)—R⁴², wherein R⁴² is C₁₋₆ alkyl, C₃₋₆ cycloalkyl or C_{6 or 10} aryl; R⁴ is H or from one to three substituents at any carbon atom of said chain D, said substituent independently selected from the group consisting of: C₁₋₆ alkyl, C₁₋₆ haloalkyl, C₁₋₆ alkoxy, hydroxy, halo, amino, oxo, thio and C 1-6 thioalkyl, and A is an amide of formula —C(O)—NH—R⁵, wherein R⁵ is selected from the group consisting of: C₁₋₈ alkyl, C₃₋₆ cycloalkyl, C_{6 or 10} aryl and C₇-₁₆ aralkyl;

or A is a carboxylic acid.

In another embodiment, the compound is a compound of formula formula XXIII

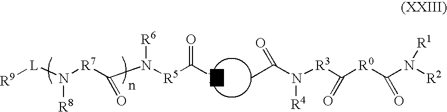

a pharmaceutically acceptable salt, solvate or ester thereof; wherein:

R⁰ is a bond or difluoromethylene;

R¹ is hydrogen, optionally substituted aliphatic group, optionally substituted cyclic group or optionally substituted aromatic group;

R² and R⁹ are each independently optionally substituted aliphatic group, optionally substituted cyclic group or optionally substituted aromatic group;

R3, R5 and R7 are each independently:
optionally substituted (1, 1- or 1,2-)cycloalkylene; or
optionally substituted (1,1- or 1,2-) heterocyclylene; or

methylene or ethylene), substituted with one substituent selected from the group consisting of an optionally substituted aliphatic group, an optionally substituted cyclic group or an optionally substituted aromatic group, and wherein the methylene or ethylene is further optionally substituted with an aliphatic group substituent; or;

R4, R 6, R8 and R¹⁰ are each independently hydrogen or optionally substituted aliphatic group;

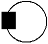

is substituted monocyclic azaheterocyclyl or optionally substituted multicyclic azaheterocyclyl, or optionally substituted multicyclic azaheterocyclenyl wherein the unsaturatation is in the ring distal to the ring bearing the R⁹-L-(N(R⁸)—R⁷—C(O)—)ₙN(R⁶)—R⁵—C(O)—N moiety and to which the —C(O)—N(R⁴)—R³—C(O)C(O)NR²R¹ moiety is attached; L is —C(O)—, —OC(O)—, —NR¹⁰C(O)—, —S(0)₂-, or —NR¹⁰S(0)₂-; and n is 0 or 1,

provided when

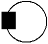

is substituted

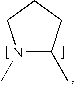

then L is —OC(O)— and R⁹ is optionally substituted aliphatic; or at least one of R³, R⁵ and R⁷ is ethylene, substituted with one substituent selected from the group consisting of an optionally substituted aliphatic group, an optionally substituted cyclic group or an optionally substituted aromatic group and wherein the ethylene is further optionally substituted with an aliphatic group substituent; or R⁴is optionally substituted aliphatic.

In another embodiment, the compound is a compound of formula formula (XXIV)

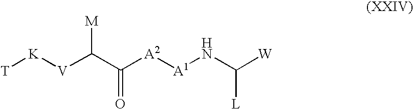

or a pharmaceutically acceptable salt, solvate or ester thereof; wherein:

W is:

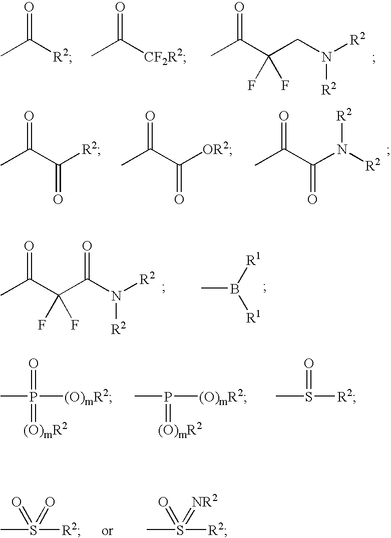

m is 0 or 1;

each R¹ is hydroxy, alkoxy, or aryloxy, or each R¹ is an oxygen atom and together with the boron, to which they are each bound, form a 5-7 membered ring, wherein the ring atoms are carbon, nitrogen, or oxygen;

each R² is independently hydrogen, alkyl, alkenyl, aryl, aralkyl, aralkenyl, cycloalkyl, cycloalkylalkyl, cycloalkenyl, cycloalkenylalkyl, heterocyclyl, heterocyclylalkyl, heterocyclylalkenyl, heteroaryl, or heteroaralkyl, or two R² groups, which are bound to the same nitrogen atom, form together with that nitrogen atom, a 5-7 membered monocyclic heterocyclic ring system; wherein any R² carbon atom is optionally substituted with J;

J is alkyl, aryl, aralkyl, alkoxy, aryloxy, aralkoxy, cycloalkyl, cycloalkoxy, heterocyclyl, heterocyclyloxy, heterocyclylalkyl, keto, hydroxy, amino, alkylamino, alkanoylamino, aroylamino, aralkanoylamino, carboxy, carboxyalkyl, carboxamidoalkyl, halo, cyano, nitro, formyl, acyl, sulfonyl, or sulfonamido and is optionally substituted with 1-3 J¹ groups;

J¹ is alkyl, aryl, aralkyl, alkoxy, aryloxy, heterocyclyl, heterocyclyloxy, keto, hydroxy, amino, alkanoylamino, aroylamino, carboxy, carboxyalkyl, carboxamidoaikyl, halo, cyano, nitro, formyl, sulfonyl, or sulfonamido;

L is alkyl, alkenyl, or alkynyl, wherein any hydrogen is optionally substituted with halogen, and wherein any hydrogen or halogen atom bound to any terminal carbon atom is optionally substituted with sulfhydryl or hydroxy;

A¹ is a bond;

R⁴ is alkyl, cycloalkyl, aryl, aralkyl, heterocyclyl, heterocyclylalkyl, heteroaryl, heteroaralkyl, carboxyalkyl, or carboxamidoalkyl, and is optionally substituted with 1-3 J groups;

R⁵ and R⁶ are independently hydrogen, alkyl, alkenyl, aryl, aralkyl, aralkenyl, cycloalkyl, cycloalkylalkyl, cycloalkenyl, heterocyclyl, heterocyclylalkyl, heteroaryl, or heteroaralkyl, and is optionally substituted with 1-3 J groups;

X is a bond, —C(H)(R7)-, -0- , —S—, or —N(R8)—;

R⁷ is hydrogen, alkyl, alkenyl, aryl, aralkyl, heterocyclyl, heterocyclylalkyl, heteroaryl, or heteroaralkyl, and is optionally substititued with 1-3 J groups;

R⁸ is hydrogen alkyl, aryl, aralkyl, heterocyclyl, heterocyclylalkyl, heteroaryl, heteroaralkyl, aralkanoyl, heterocyclanoyl, heteroaralkanoyl, —C(O)R¹⁴, —SO₂R¹⁴, or carboxamido, and is optionally substititued with 1-3 J groups; or R⁸ and Z, together with the atoms to which they are bound, form a nitrogen containing mono- or bicyclic ring system optionally substituted with 1-3 J groups;

R¹⁴ is alkyl, aryl, aralkyl, heterocyclyl, heterocyclyalkyl, heteroaryl, or heteroaralkyl;

Y is a bond, —CH₂—, —C(O)—, —C(O)C(O)—, —S(O)—, —S(0)₂-, or —S(O)(NR⁷)—, wherein R⁷ is as defined above;

Z is alkyl, aryl, aralkyl, cycloalkyl, cycloalkylalkyl, heterocyclyl, heterocyclylalkyl, heteroaryl, heteroaralkyl, —OR², or —N(R²)₂, wherein any carbon atom is optionally substituted with J, wherein R² is as defined above;

A² is a bond or

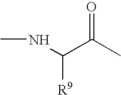

R⁹ is alkyl, cycloalkyl, aryl, aralkyl, heterocyclyl, heterocyclylalkyl, heteroaryl, heteroaralkyl, carboxyalkyl, or carboxamidoalkyl, and is optionally substituted with 1-3 J groups;

M is alkyl, cycloalkyl, aryl, aralkyl, heterocyclyl, heterocyclylalkyl, heteroaryl, or heteroaralkyl, optionally substituted by 1-3 J groups, wherein any alkyl carbon atom may be replaced by a heteroatom;

V is a bond, —CH₂—, —C(H)(R¹¹)—, -0-, —S—, or —N(R¹¹)—;

R¹¹ is hydrogen or C₁₋₃ alkyl;

K is a bond, -0-, —S—, —C(O)—, —S(O)—, —S(0)₂-, or —S(O)(NR¹¹)—, wherein R¹¹ is as defined above;

T is —R¹², -alkyl-R¹², -alkenyl-R¹², -alkynyl-R¹², —OR¹², —N(R¹²)2, —C(O)R¹², —C(═NO alkyl)R¹²₁ or

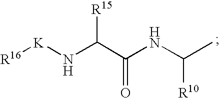

R¹² is hydrogen, aryl, heteroaryl, cycloalkyl, heterocyclyl, cycloalkylidenyl, or heterocycloalkylidenyl, and is optionally substituted with 1-3 J groups, or a first R¹² and a second R¹², together with the nitrogen to which they are bound, form a mono- or bicyclic ring system optionally substituted by 1-3 J groups;

R¹⁰ is alkyl, cycloalkyl, aryl, aralkyl, heterocyclyl, heterocyclylalkyl, heteroaryl, heteroaralkyl, carboxyalkyl, or carboxamidoalkyl, and is optionally substituted with 1-3 hydrogens J groups;

R¹⁵ is alkyl, cycloalkyl, aryl, aralkyl, heterocyclyl, heterocyclylalkyl, heteroaryl, heteroaralkyl, carboxyalkyl, or carboxamidoalkyl, and is optionally substituted with 1-3 J groups; and

R¹⁶ is hydrogen, alkyl, aryl, heteroaryl, cycloalkyl, or heterocyclyl.

In another embodiment, the compound is a compound of formula XXV

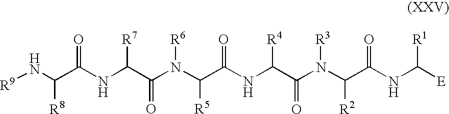

or a pharmaceutically acceptable salt, solvate or ester thereof;

wherein

E represents CHO or B(OH)2;

R¹ represents lower alkyl, halo-lower alkyl, cyano-lower alkyl, lower alkylthio-lower alkyl, aryl-lower alkylthio-lower alkyl, aryl-lower alkyl, heteroaryllower alkyl, lower alkenyl or lower alkynyl;

R² represents lower alkyl, hydroxy-lower alkyl, carboxylower alkyl, aryl-lower alkyl, aminocarbonyl-lower alkyl or lower cycloalkyl-lower alkyl; and

R³ represents hydrogen or lower alkyl;

or R² and R³ together represent di- or trimethylene optionally substituted by hydroxy;

R⁴ represents lower alkyl, hydroxy-lower alkyl, lower cycloalkyl-lower alkyl, carboxy-lower alkyl, aryllower alkyl, lower alkylthio-lower alkyl, cyano-lower alkylthio-lower alkyl, aryl-lower alkylthio-lower alkyl, lower alkenyl, aryl or lower cycloalkyl;

R⁵ represents lower alkyl, hydroxy-lower alkyl, lower alkylthio-lower alkyl, aryl-lower alkyl, aryl-lower alkylthio-lower alkyl, cyano-lower alkylthio-lower alkyl or lower cycloalkyl;

R⁶ represents hydrogen or lower alkyl;

R⁷ represent lower alkyl, hydroxydower alkyl, carboxylower alkyl, aryl-iower alkyl, lower cycloalkyl-lower alkyl or lower cycloalkyl;

R⁸ represents lower alkyl, hydroxy-lower alkyl, carboxylower alkyl or aryl-lower alkyl; and

R⁹ represents lower alkylcarbonyl, carboxy-lower alkylcarbonyl, arylcarbonyl, lower alkylsulphonyl, arylsulphonyl, lower alkoxycarbonyl or aryl-lower alkoxycarbonyl.

In another embodiment, the compound is a compound of formula XXVI

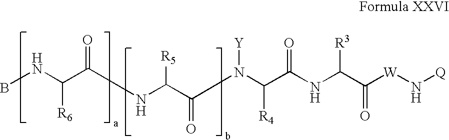

or a pharmaceutically acceptable salt, solvate or ester thereof; wherein

B is an acyl derivative of formula R₁₁—C(O)— wherein R₁₁ is Cl-10 alkyl optionally substituted with carboxyl; or R₁₁ is C₆ or C₁₀ aryl or C₇₋₁₆ aralkyl optionally substituted with a C₁₋₆alkyl;

a is 0 or 1;

R₆, when present, is carboxy(lower)alkyl;

b is 0 or 1;

R₅, when present, is C₁₋₆ alkyl, or carboxy(lower)alkyl;

Y is H or C₁₋₆ alkyl;

R₄ is C₁₋₁₀ alkyl; C₃₋₁₀ cycloalkyl;

R₃ is C1-10 alkyl; C₃₋₁₀ cycloalkyl;

W is a group of formula:

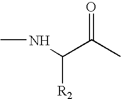

wherein R₂ is C₁₋₁₀ alkyl or C₃₋₇ cycloalkyl optionally substituted with carboxyl; C₆ or C₁₀ aryl; or C₇₋₁₆ aralkyl; or

W is a group of formula:

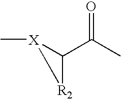

wherein X is CH or N; and

R₂′ is C₃₋₄ alkylene that joins X to form a 5- or 6-membered ring, said ring optionally substituted with OH; SH; NH2; carboxyl; R₁₂; OR₁₂, SR₁₂, NHR₁₂ or NR₁₂R₁₂′ wherein R₁₂ and R₁₂′ are independently:

cyclic C₃₋₁₆ alkyl or acyclic C₁₋₁₆ alkyl or cyclic C₃₋₁₆ alkenyl or acyclic C₂₋₁₆ alkenyl, said alkyl or alkenyl optionally substituted with NH₂, OH, SH, halo, or carboxyl; said alkyl or alkenyl optionally containing at least one heteroatom selected independently from the group consisting of: 0, S, and N; or

R₁₂ and R₁₂′ are independently C₆ or C₁₀ aryl or C₇₋₁₆ aralkyl optionally substituted with C₁₋₆ alkyl, NH₂, OH, SH, halo, carboxyl or carboxy(lower)alkyl; said aryl or aralkyl optionally containing at least one heteroatom selected independently from the group consisting of: 0, S, and N;

said cyclic alkyl, cyclic alkenyl, aryl or aralkyl being optionally fused with a second 5-, 6-, or 7-membered ring to form a cyclic system or heterocycle, said second ring being optionally substituted with NH₂. OH, SH, halo, carboxyl or carboxy(lower)alkyl; C₆ or C₁₀ aryl, or heterocycle; said second ring optionally containing at least one heteroatom selected independently from the group consisting of: 0, S, and N;

Q is a group of the formula:

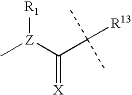

wherein Z is CH or N;

X is 0 or S;

R₁ is H, C₁₋₆ alkyl or C₁₋₆ alkenyl both optionally substituted with thio or halo; and

when Z is CH, then R₁₃ is H; CF₃; CF₂CF₃; CH₂—R₁₄; CH(F)—R₁₄; CF₂—R₁₄; NR₁₄R₁₄′; S—R₁₄; or C0-NH—R₁₄ wherein R₁₄ and R₁₄′ are independently hydrogen, cyclic C₃₋₁₀ alkyl or acyclic C₁₋₁₀ alkyl or cyclic C₃₋₁₀ alkenyl or acyclic C₂₋₁₀ alkenyl, said alkyl or alkenyl optionally substituted with NH₂, OH, SH, halo or carboxyl; said alkyl or alkenyl optionally containing at least one heteroatom selected independently from the group consisting of: 0, S, and N; or

R₁₄ and R₁₄′ are independently C₆ or C₁₀ aryl or C₇₋₁₆ aralkyl optionally substituted with C₁₋₆ alkyl, NH₂, OH, SH, halo, carboxyl or carboxy(lower)alkyl or substituted with a further C₃₋₇ cycloalkyl, C₆ or C₁₀ aryl, or heterocycle; said aryl or aralkyl optionally containing at least one heteroatom selected independently from the group consisting of: 0, S, and N;

said cyclic alkyl, cyclic alkenyl, aryl or aralkyl being optionally fused with a second 5-, 6-, or 7-membered ring to form a cyclic system or heterocycle, said second ring being optionally substituted with NH₂, OH, SH, halo, carboxyl or carboxy(lower)alkyl or substituted with a further C₃₋₇ cycloalkyl, C₆ or C₁₀ aryl, or heterocycle; said second ring optionally containing at least one heteroatom selected independently from the group consisting of: 0, S, and N;

or R₁₄ and R₁₄′ are independently C₁₋₄ alkyl which when joined together with N form a 3 to 6-membered nitrogen-containing ring which is optionally fused with a further C₃₋₇ cycloalkyl, C₆ or C₁₀ aryl or heterocycle;

with the proviso that when Z is CH, then R₁₃ is not an α-amino acid or an ester thereof;

when Z is N, then R₁₃ is H; carboxy; C₁₋₆ alkyl optionally substituted with carboxy; CH₂—R₁₄; CHR₁₄R₁₄′; CH(F)—R₁₄; O—R₁₄; NR₁₄R₁₄′ or S—R₁₄ wherein R₁₄ and R₁₄′ are as defined above; or

Q is a phosphonate group of the formula:

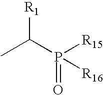

wherein R₁₅ and R₁₆ are independently C₆₋₂₀ aryloxy; and R₁ is as defined above.

In another embodiment, the compound is selected from the group consisting of:

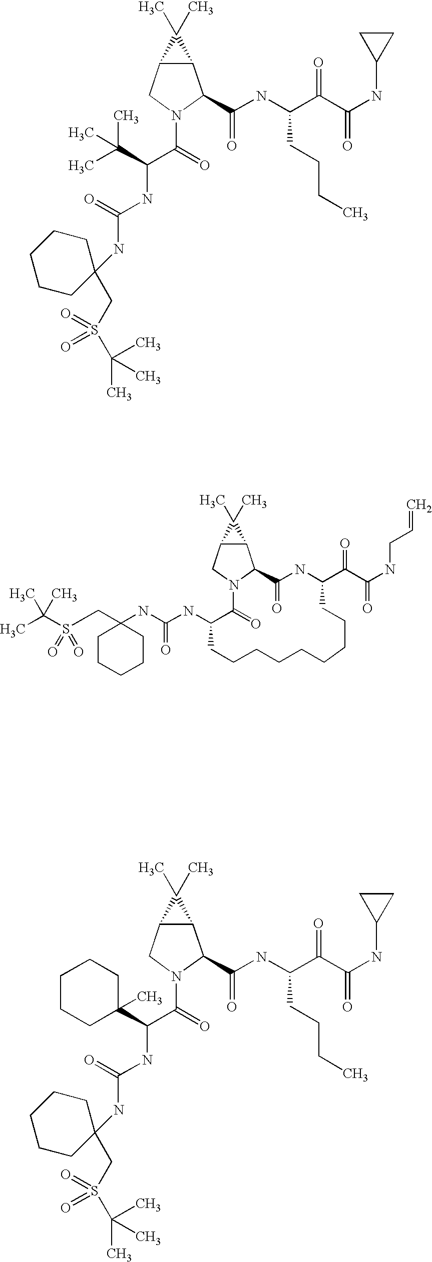

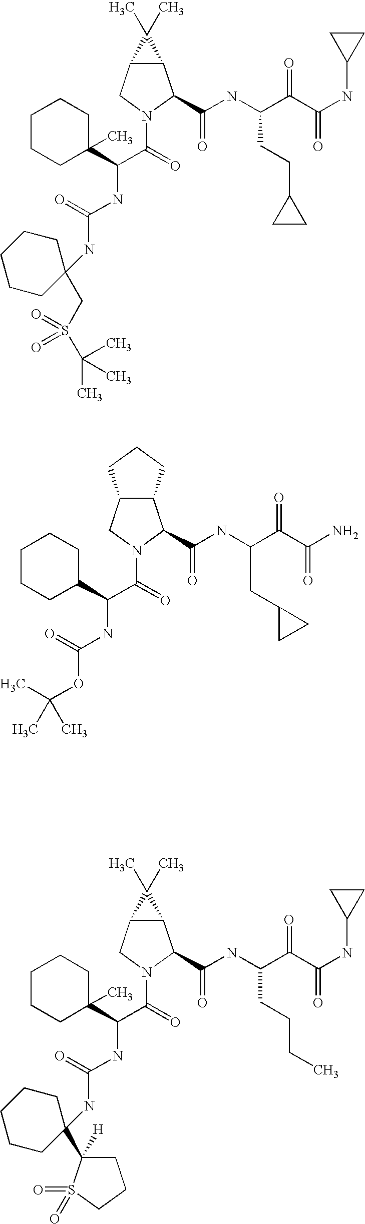

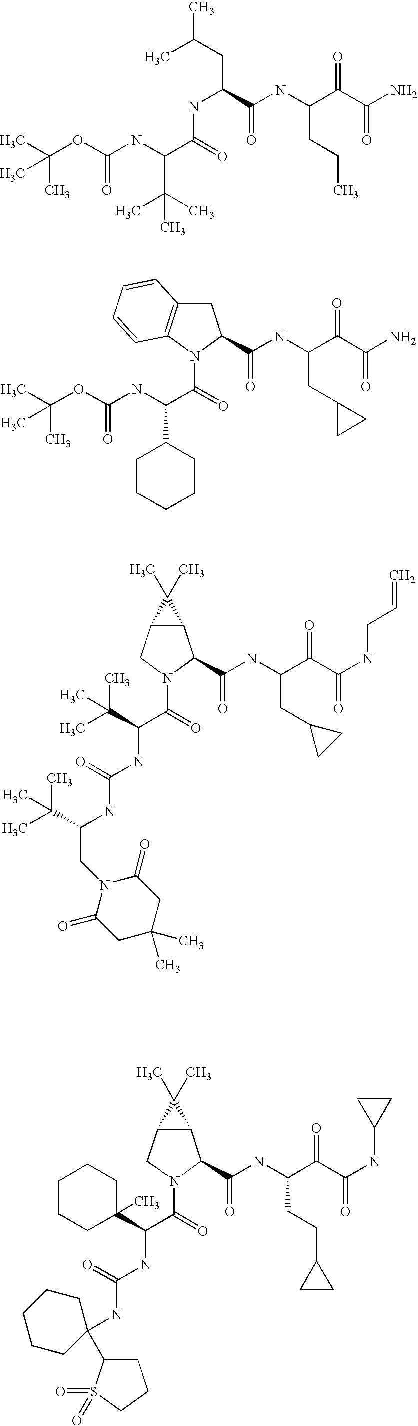

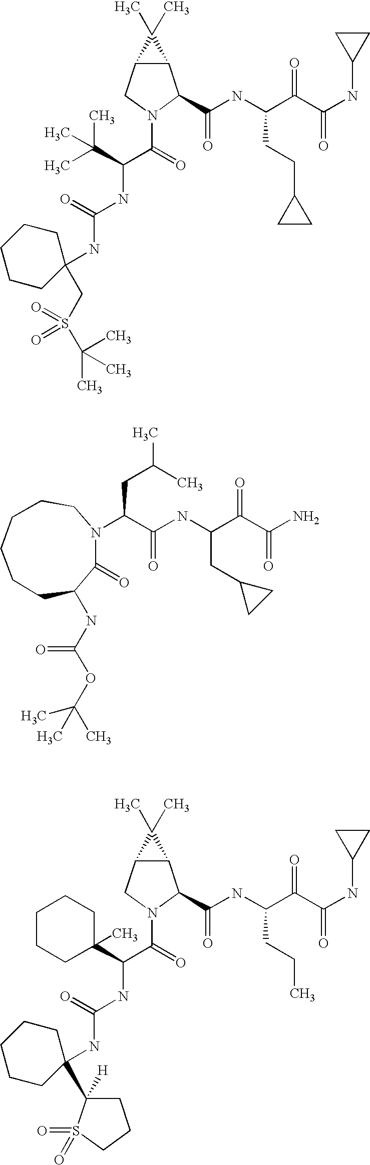

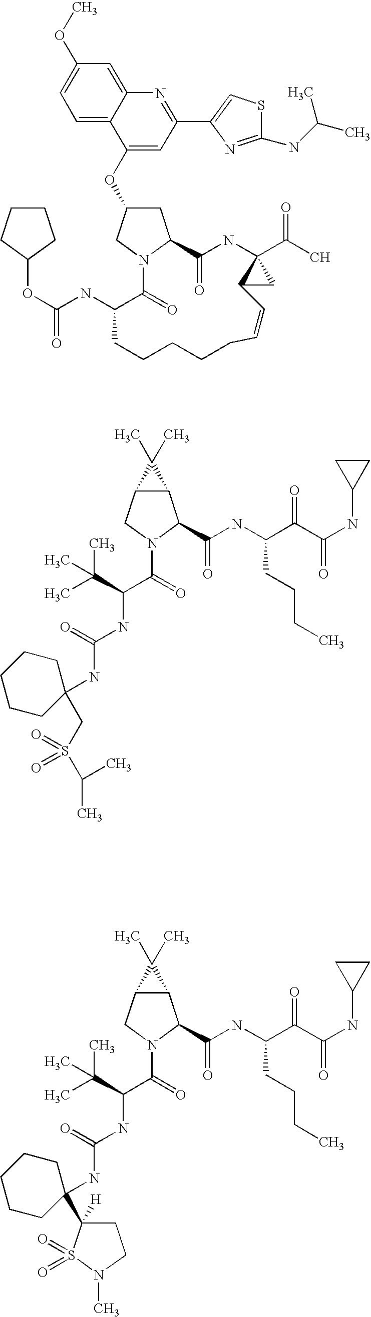

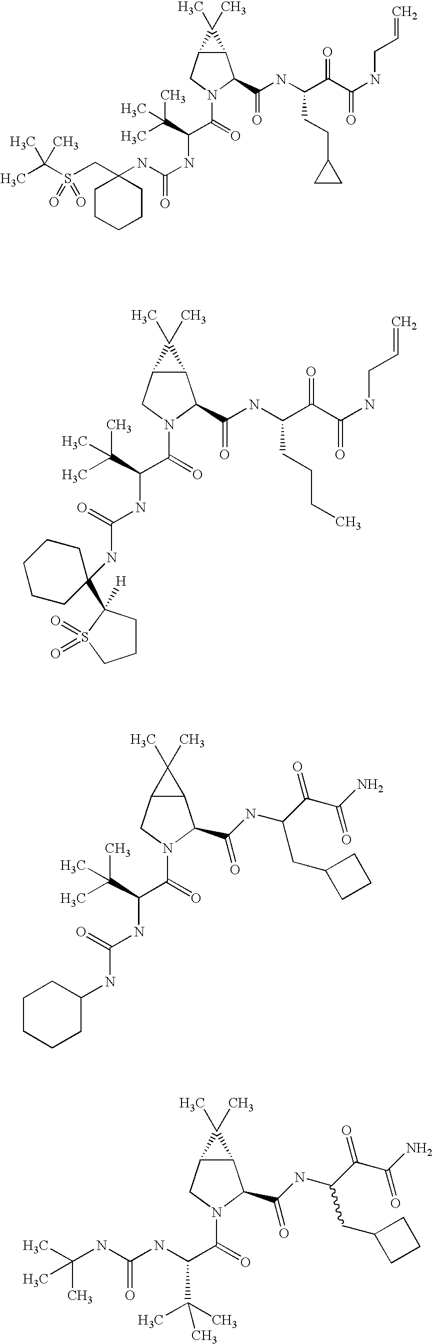

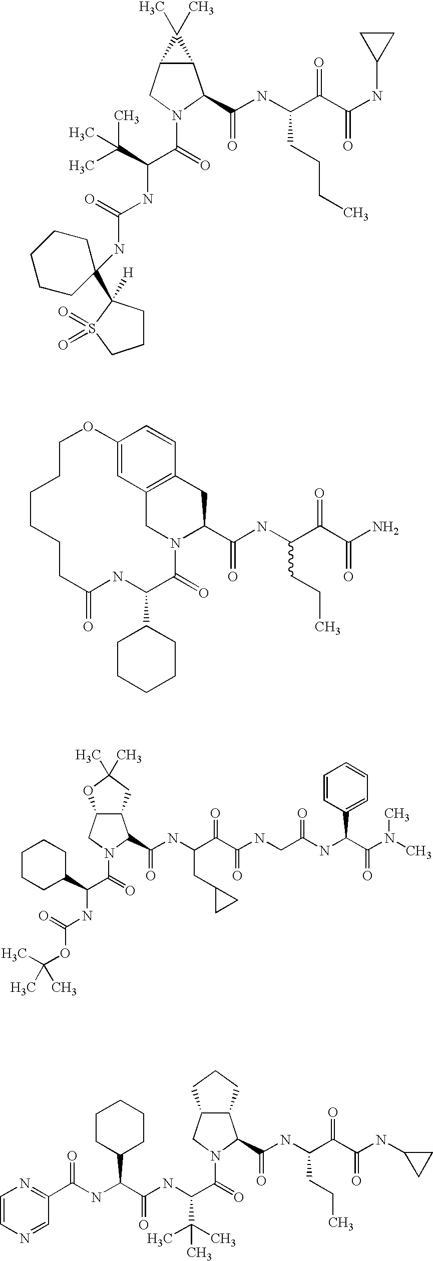

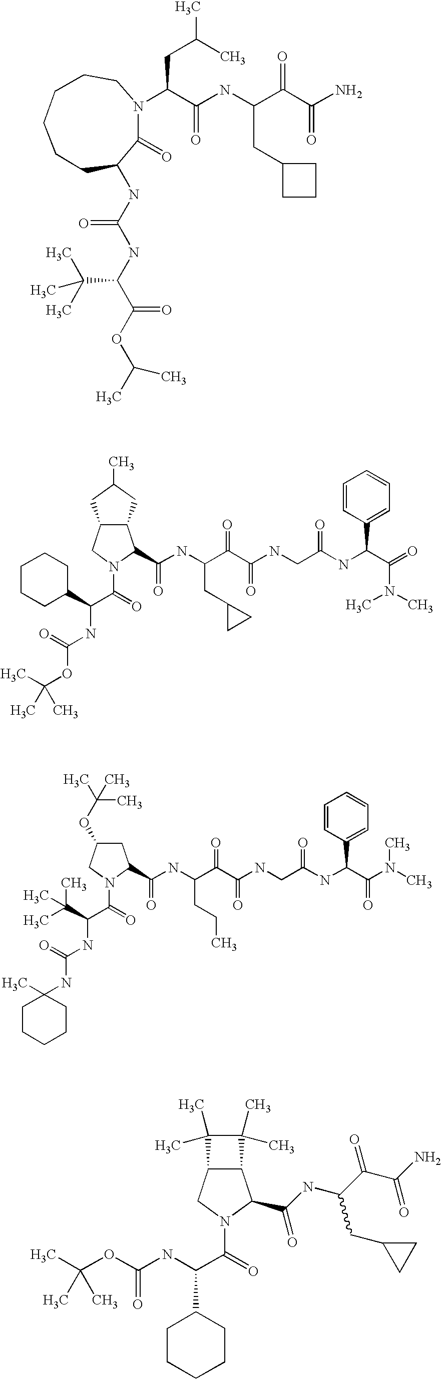

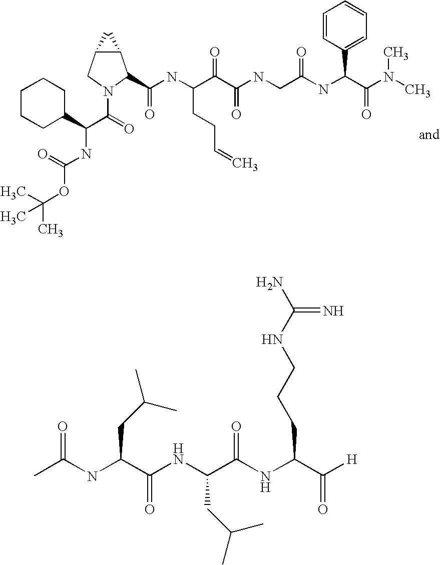

or a pharmaceutically acceptable salt, solvate or ester thereof.

Methods of treating a wide variety of diseases/disorders associated with cathepsin activity and/or for inhibiting cathepsin activity in a subject comprising administering to a subject in need of such treatment an effective amount of at least one of the inventive compounds also are provided.

One example of such disorders is proliferative diseases, such as cancer, autoimmune diseases, viral diseases, fungal diseases, neurological/neurodegenerative disorders, arthritis, inflammation, anti-proliferative (e.g., ocular retinopathy), neuronal, alopecia and cardiovascular disease. Many of these diseases and disorders are listed in U.S. Pat. No. 6,413,974, the disclosure of which is incorporated herein.

Another example of a disease that can be treated by the present compounds is an inflammatory disease, such as organ transplant rejection, graft v. host disease, arthritis, rheumatoid arthritis, inflammatory bowel disease, atopic dermatitis, psoriasis, asthma, allergies, multiple sclerosis, fixed drug eruptions, cutaneous delayed-type hypersentitivity responses, tuberculoid leprosy, type I diabetes, and viral meningitis.

Another example of a disease that can be treated by the present compounds is a cardiovascular disease.

Another example of a disease that can be treated by the present compounds is a central nervous system disease, such as depression, cognitive function disease, neurodegenerative disease such as Parkinson's disease, senile dementia such as Alzheimer's disease, and psychosis of organic origin.

Other examples of diseases that can be treated by the present compounds are diseases characterized by bone loss, such as osteoporosis; gingival diseases, such as gingivitis and periodontitis; and diseases characterized by excessive cartilage or matrix degradation, such as osteoarthritis and rheumatoid arthritis.

Other than in the operating examples, or where otherwise indicated, all numbers expressing quantities of ingredients, reaction conditions, and so forth used in the specification and claims are to be understood as being modified in all instances by the term “about.”

### DETAILED DESCRIPTION

The compounds of formula I are disclosed in PCT International publication WO03/062265 published Jul. 31, 2003 which is incorporated herein by reference. Non-limiting examples of certain compounds disclosed in these publications are: 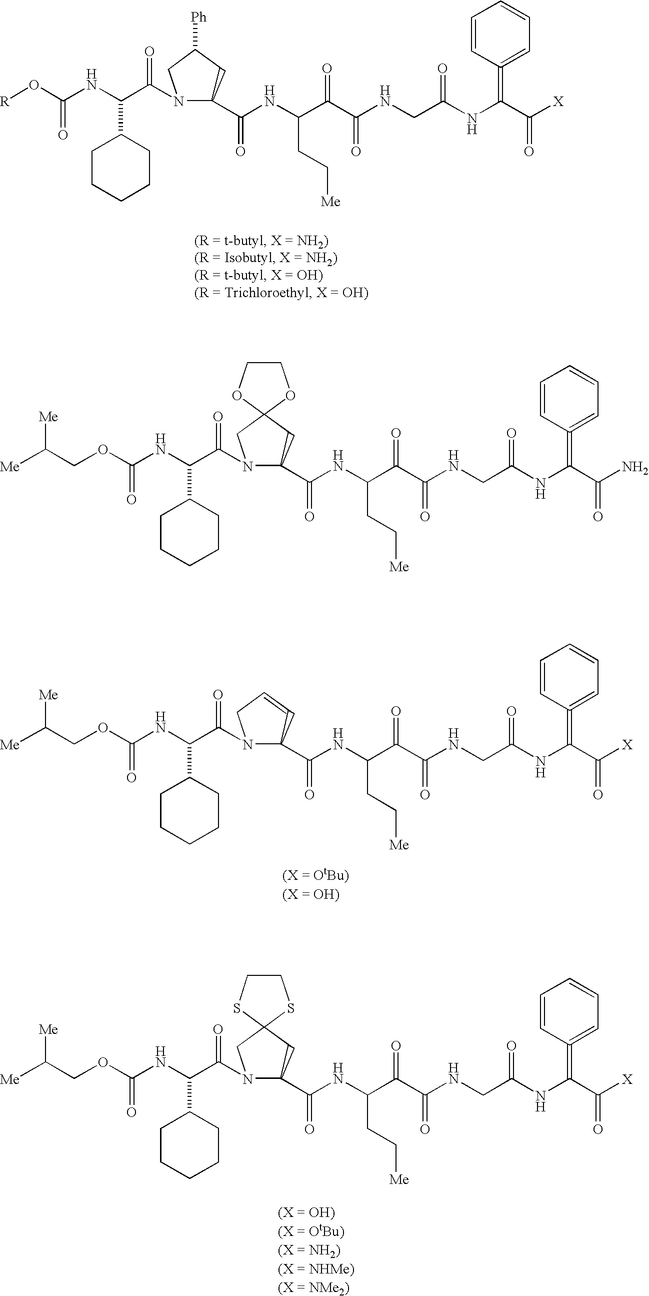 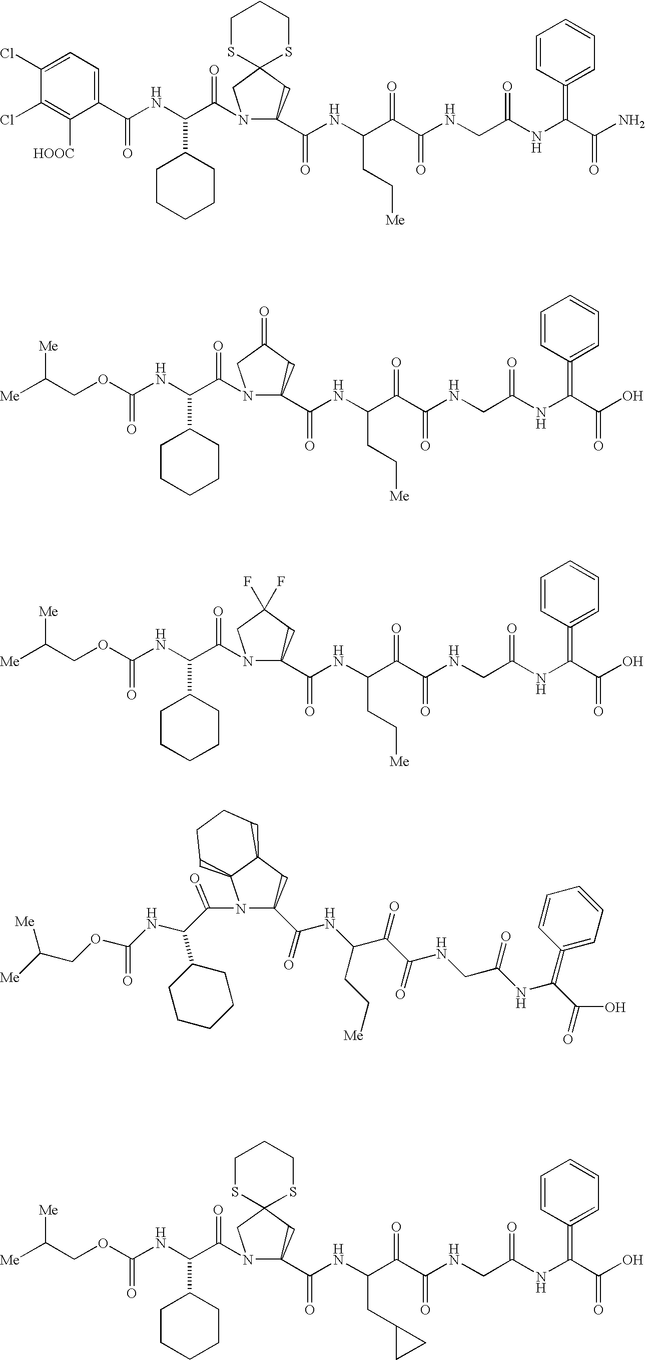 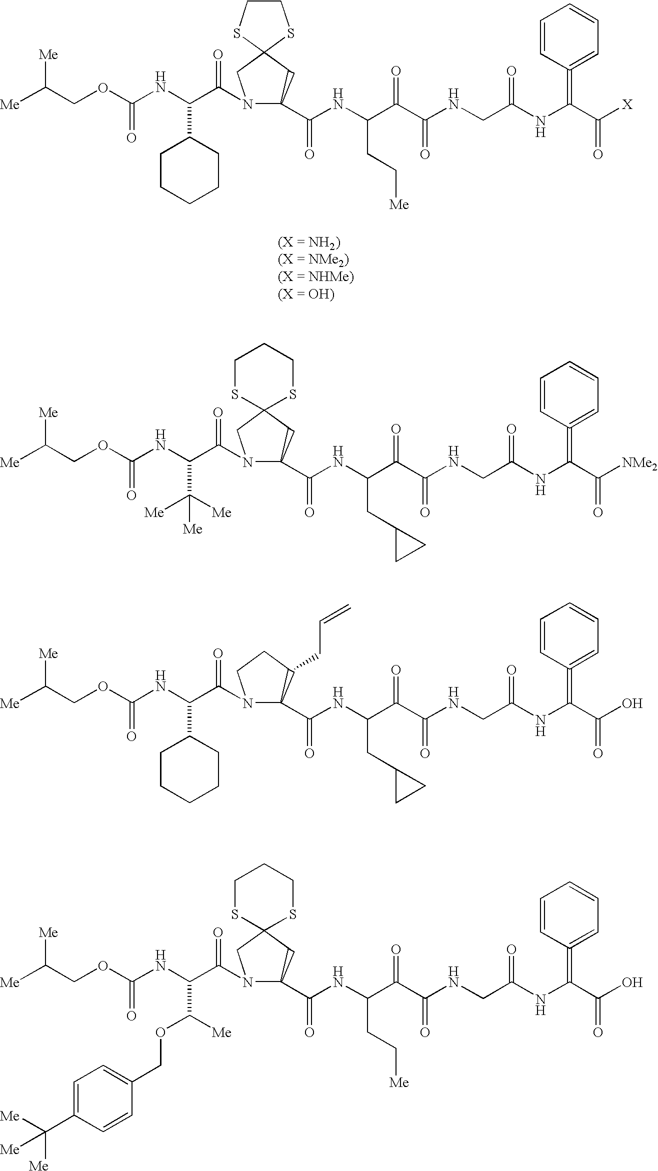 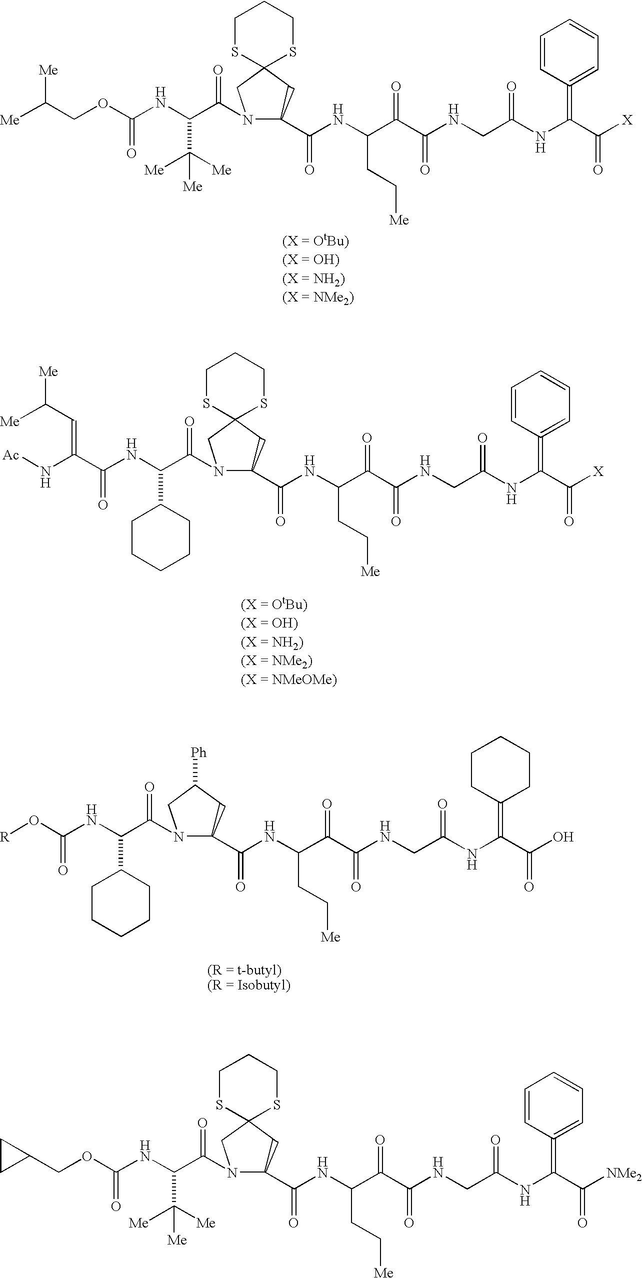 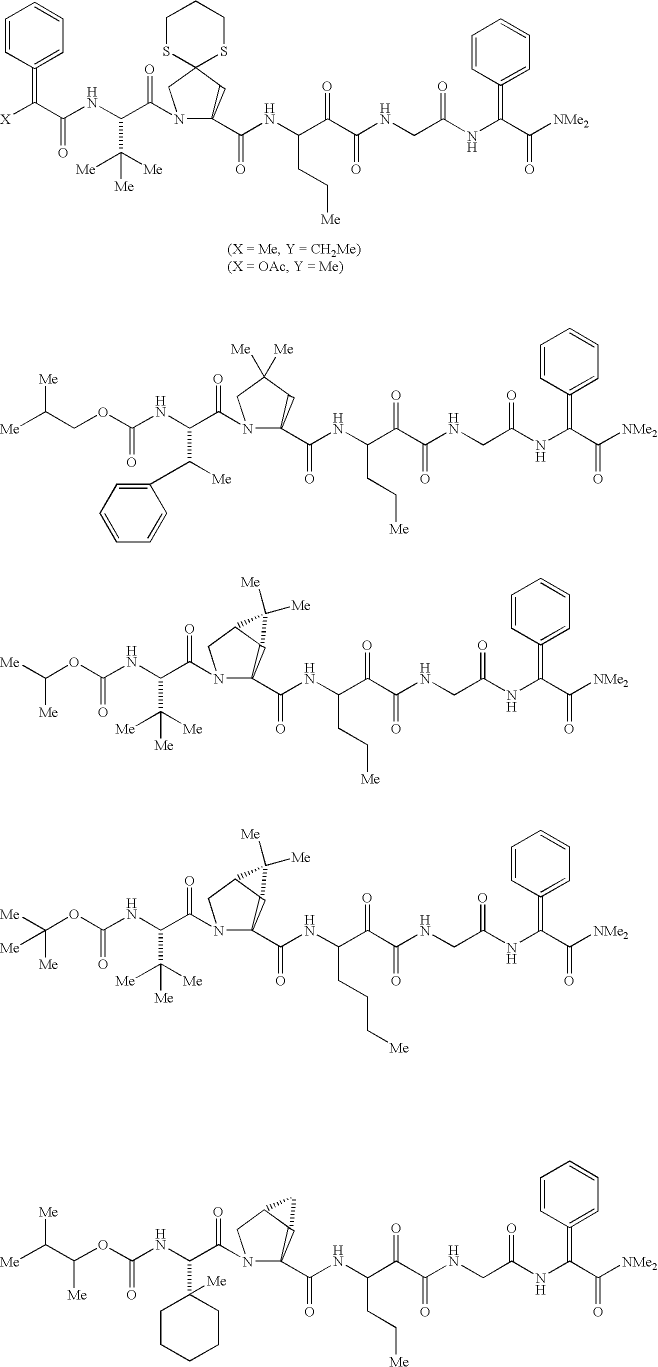 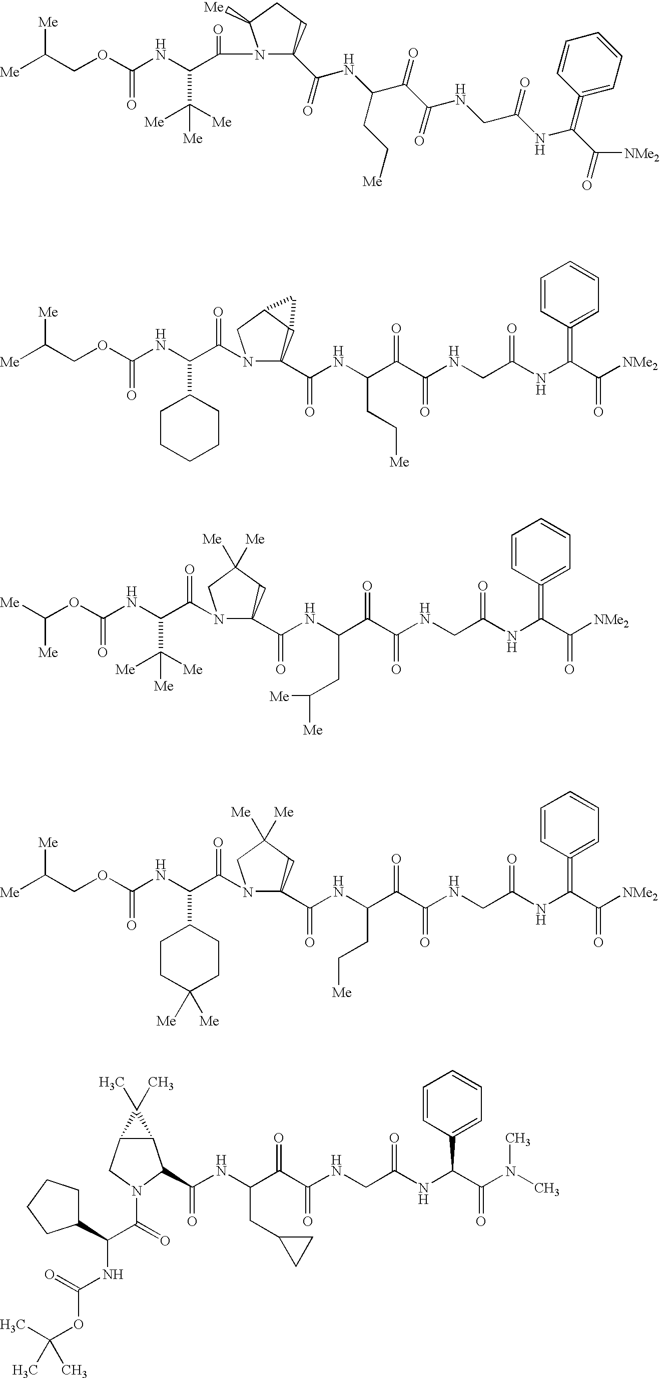 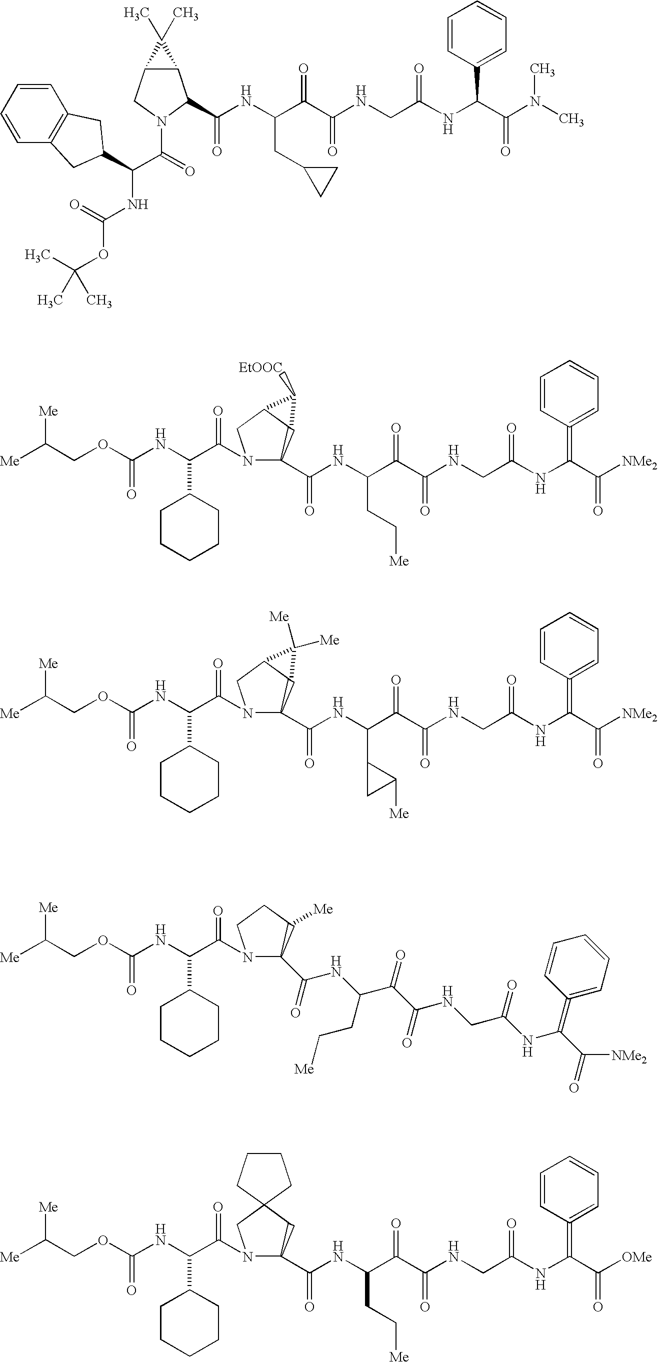 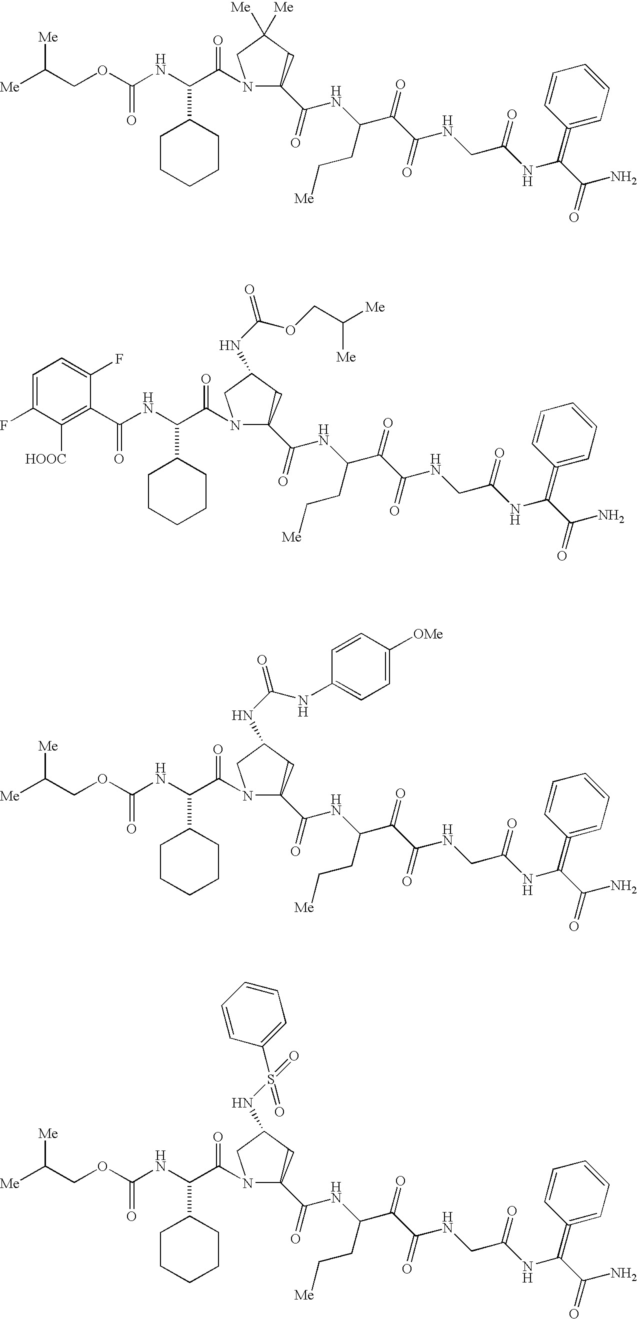 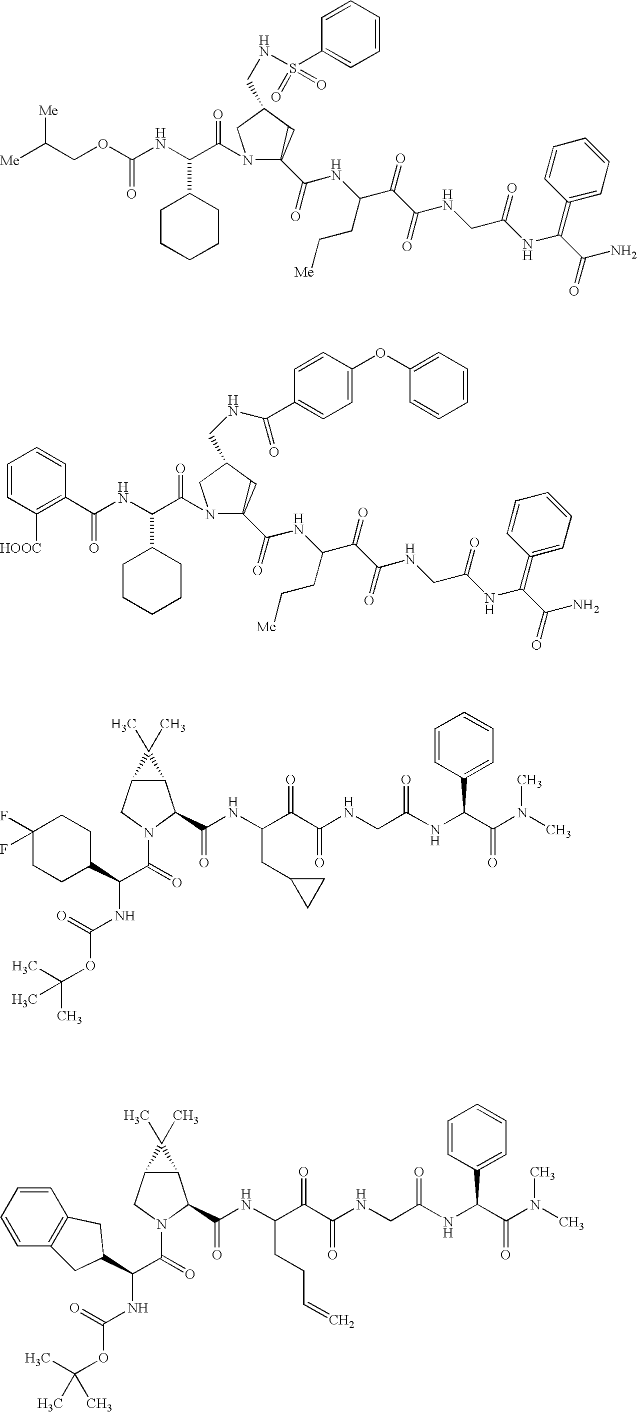 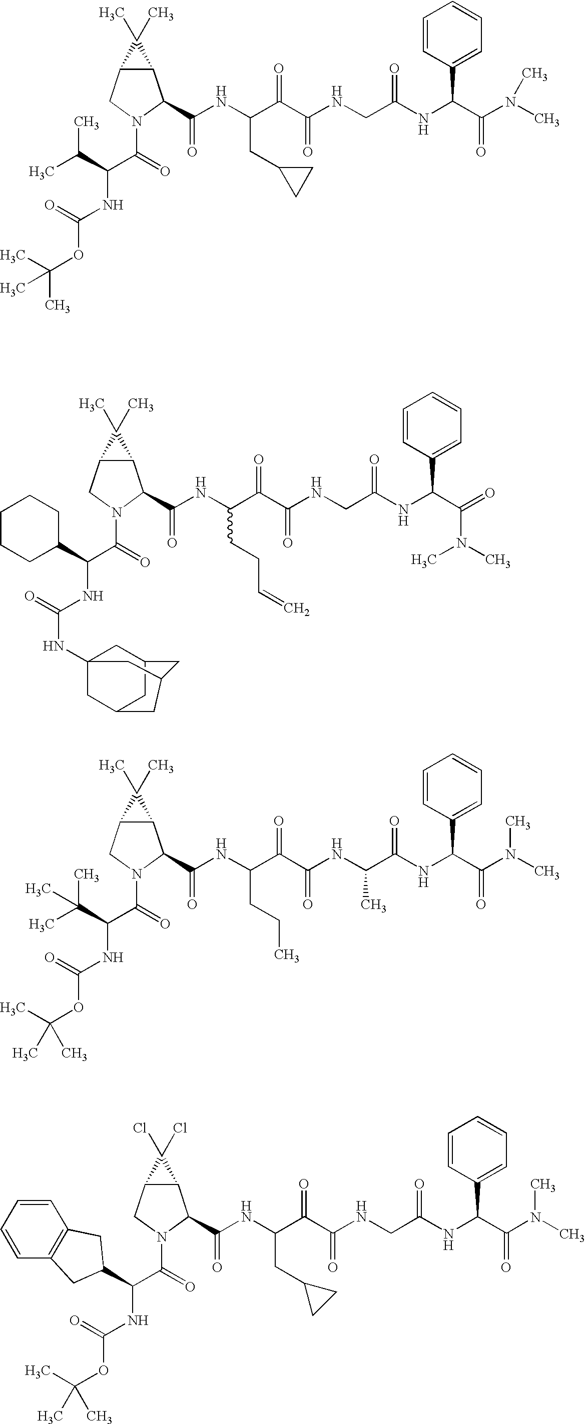 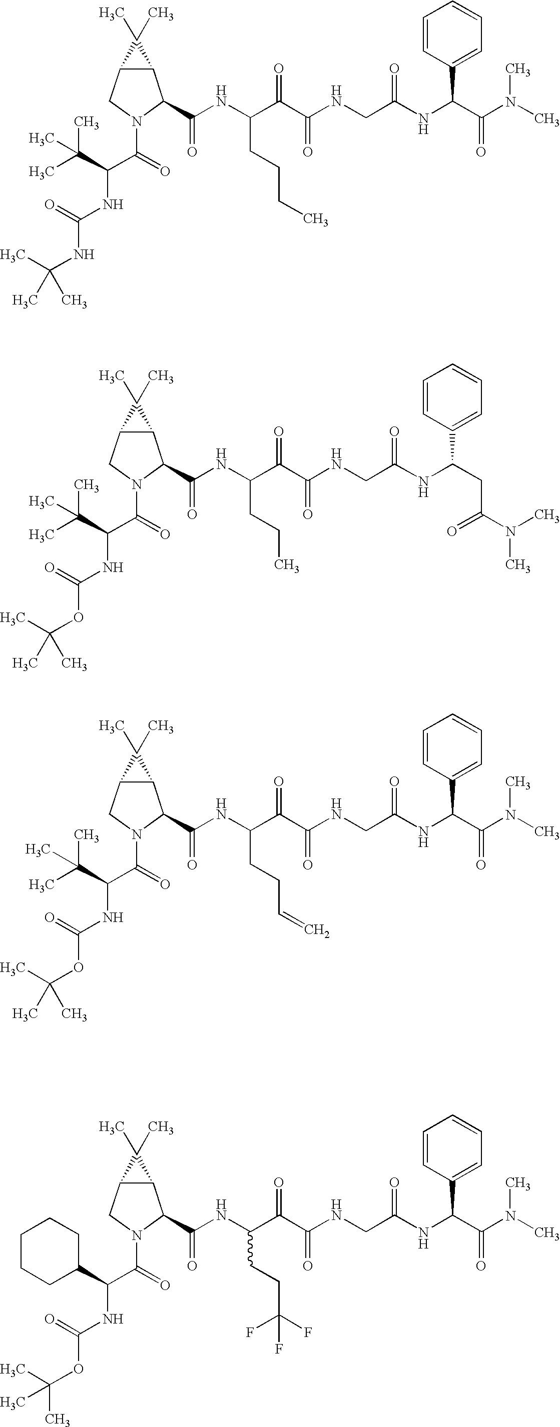 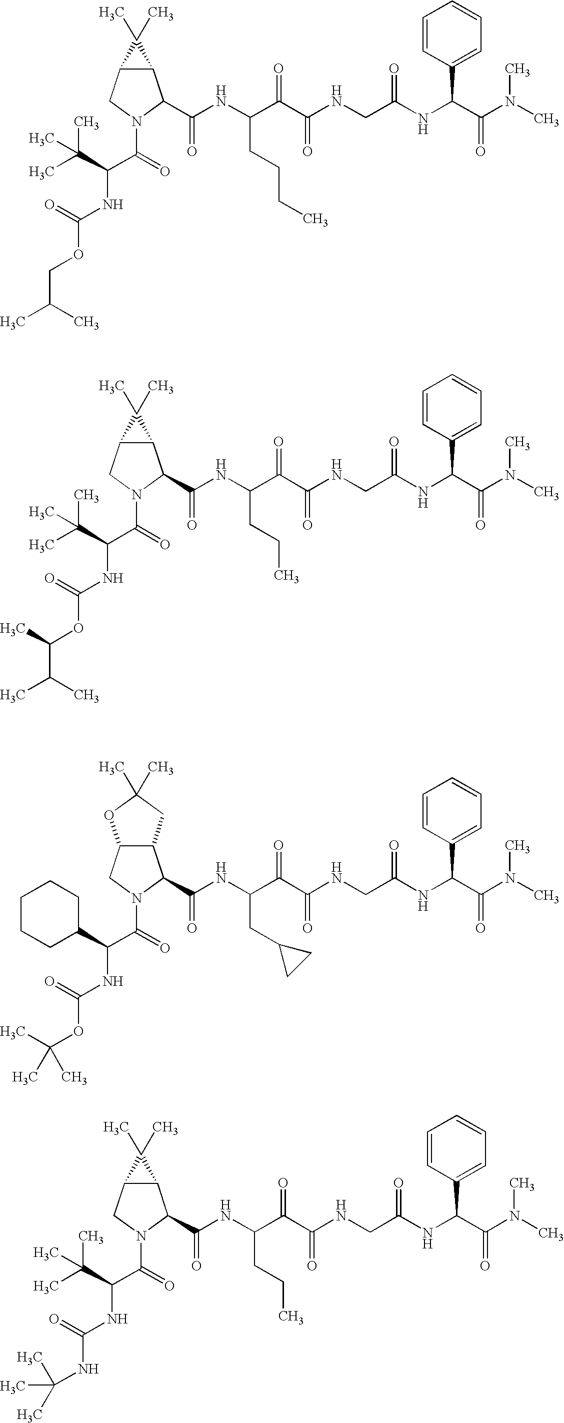 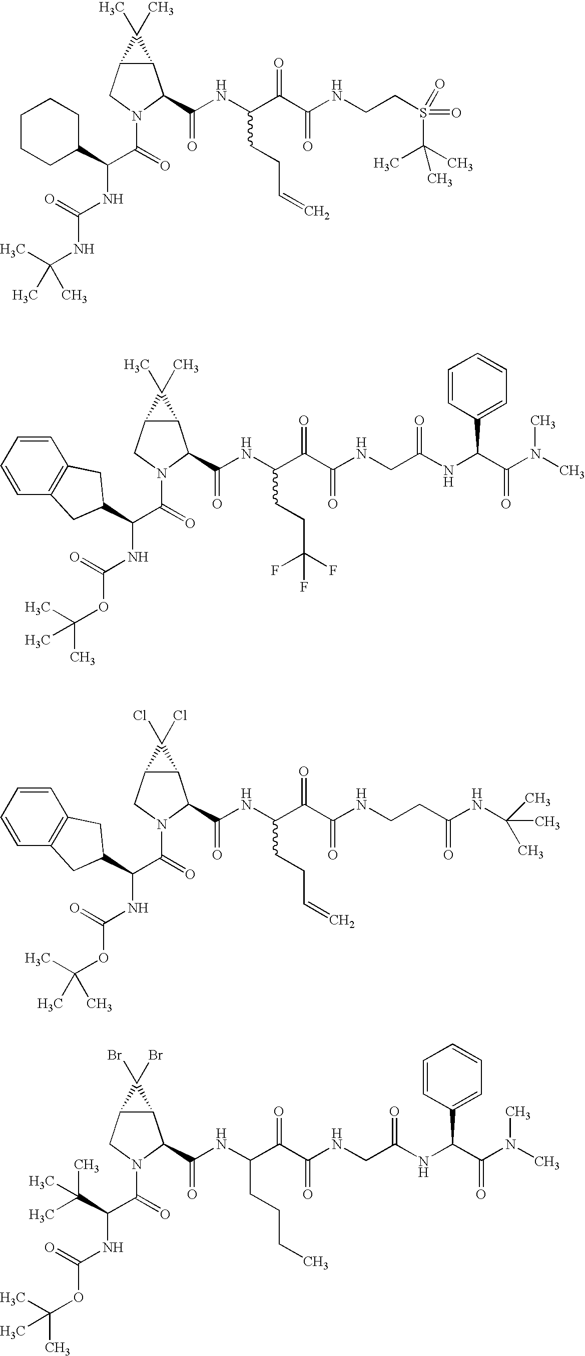 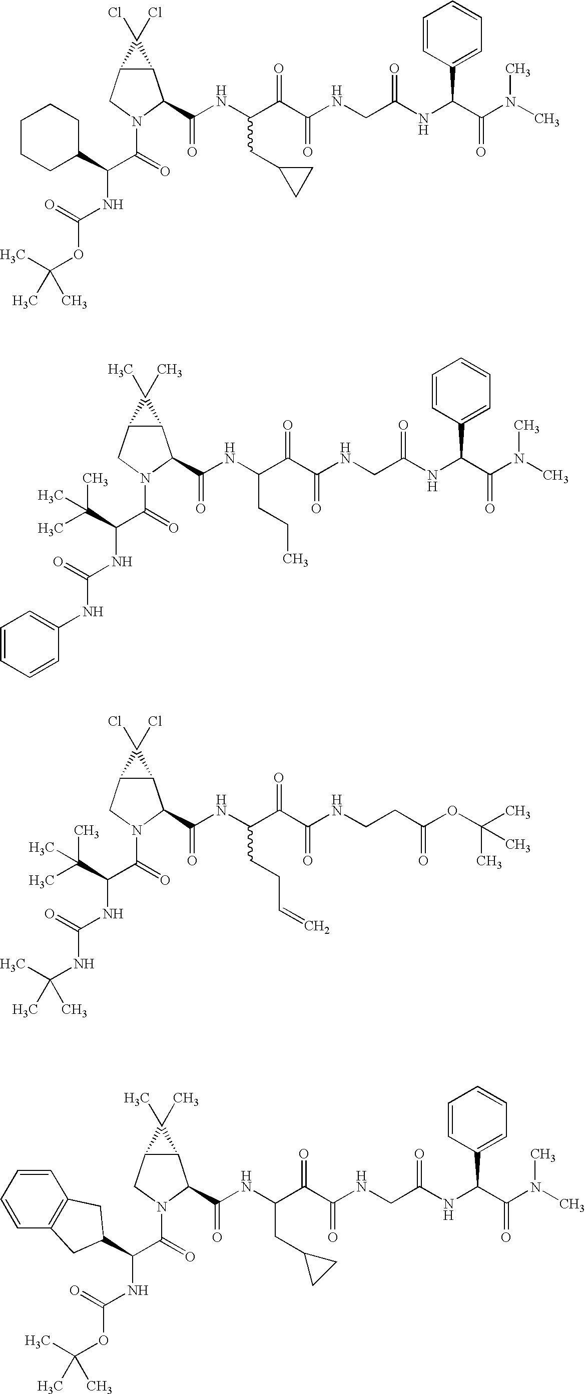 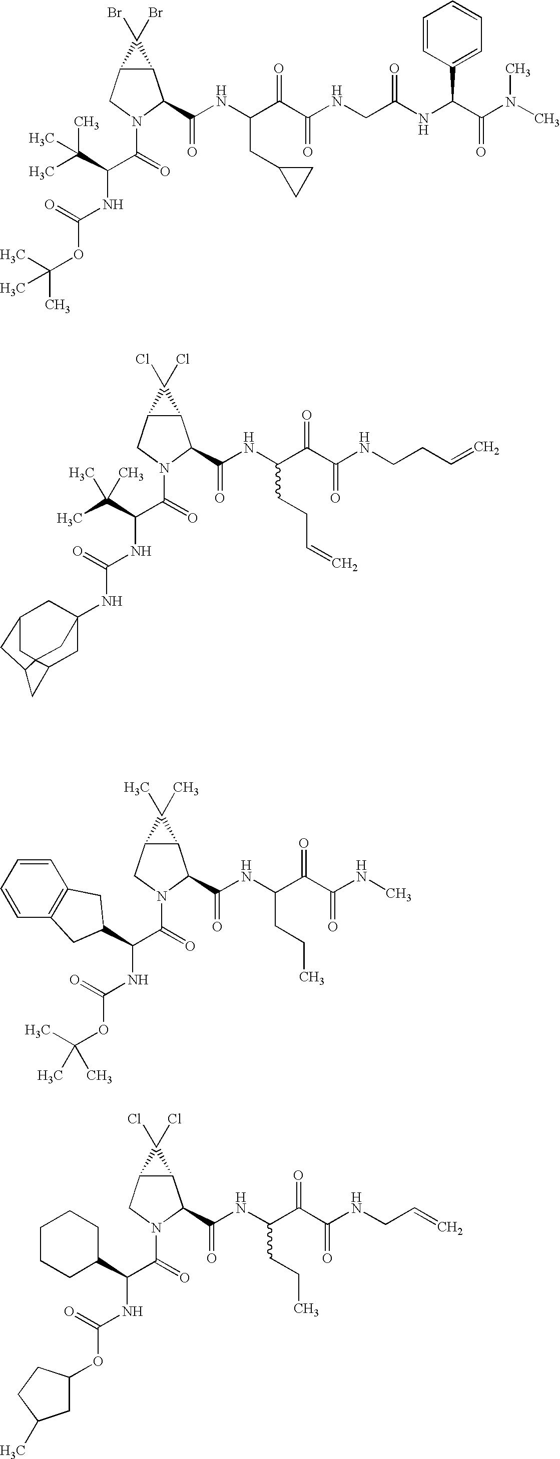 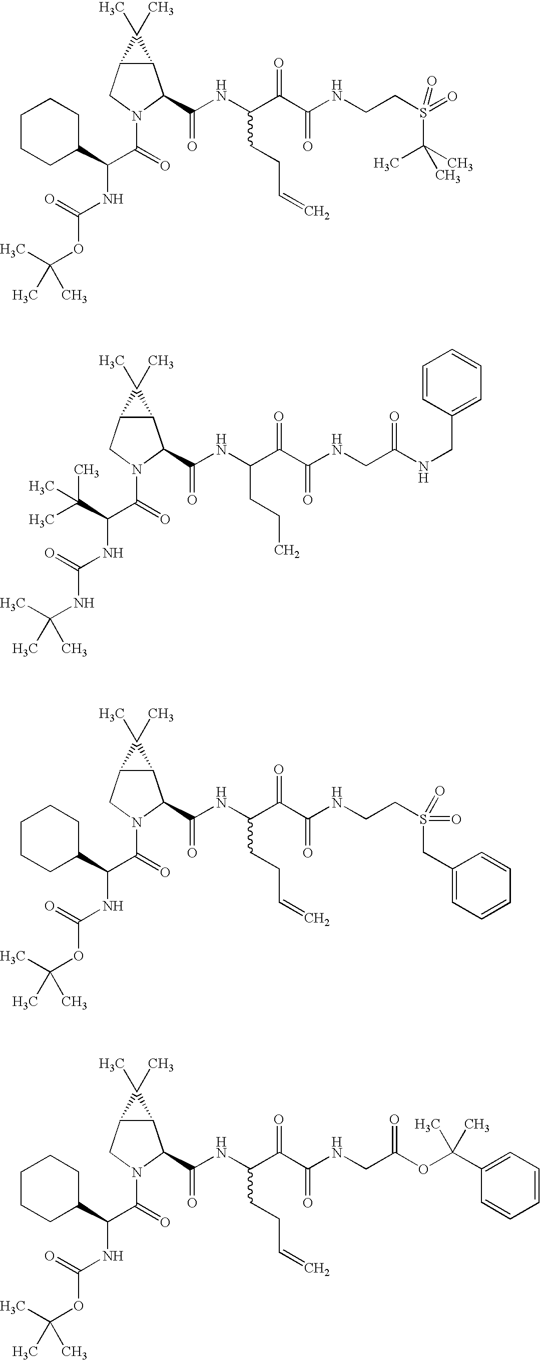 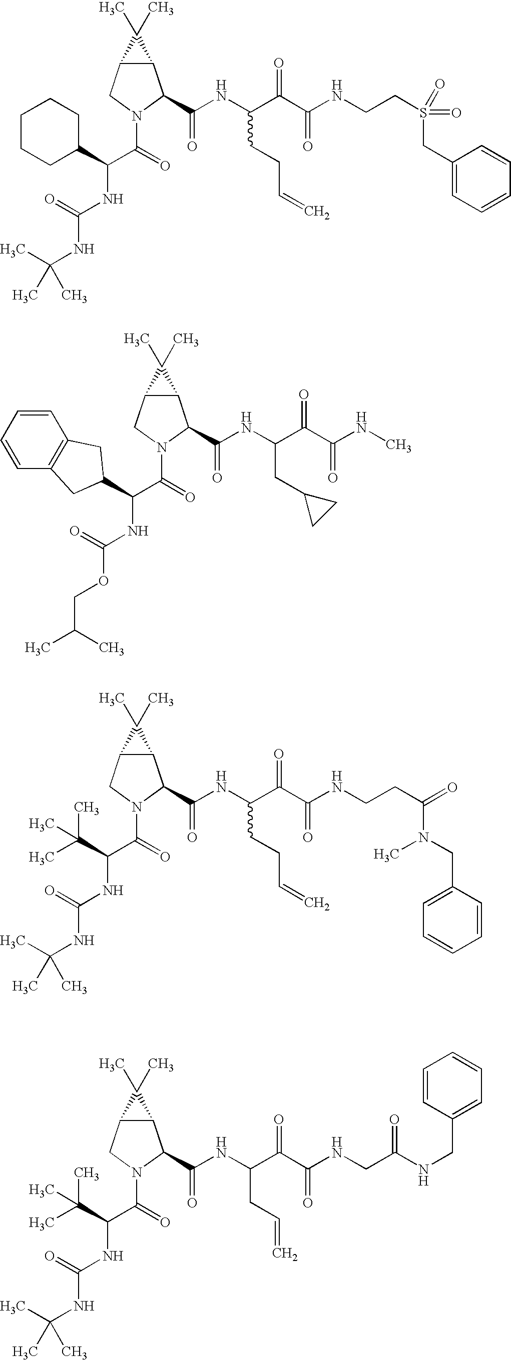 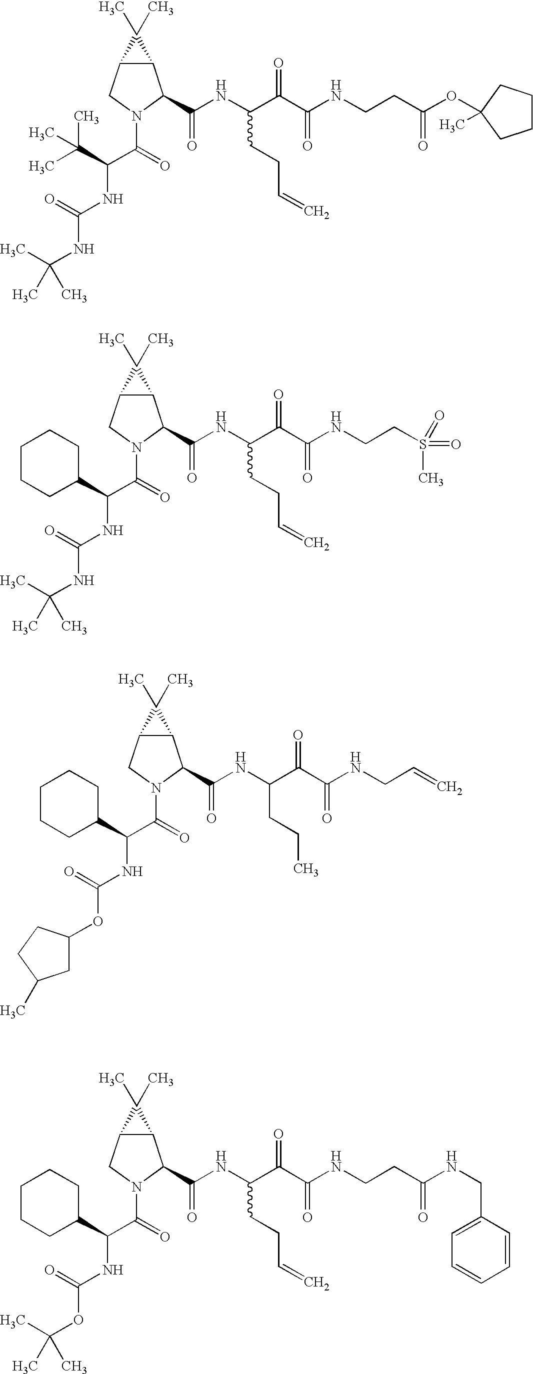 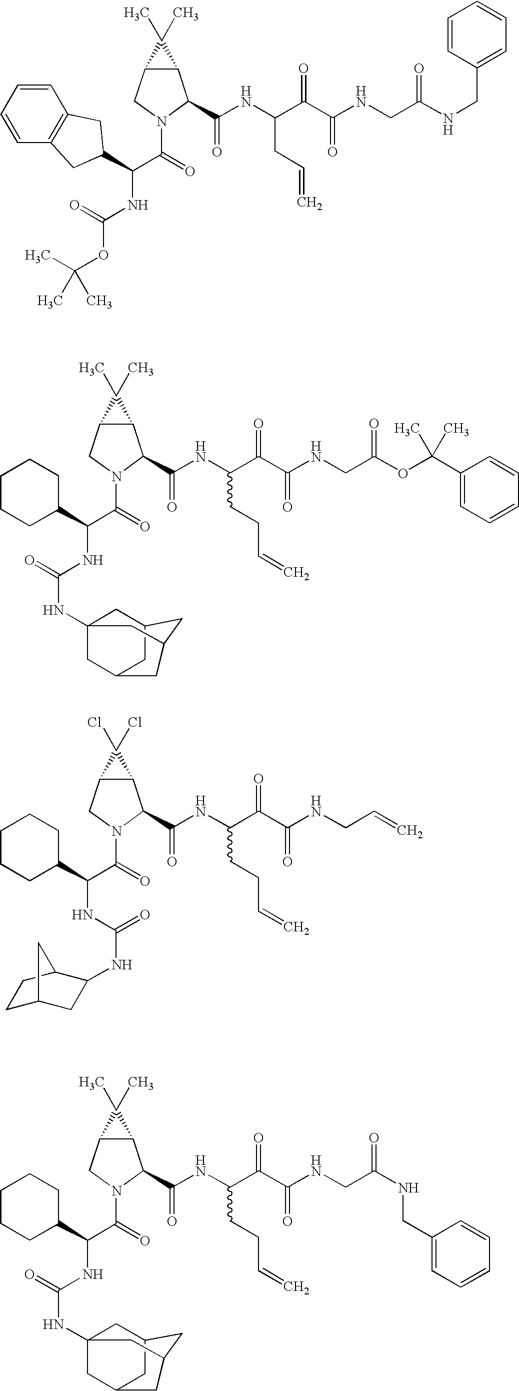 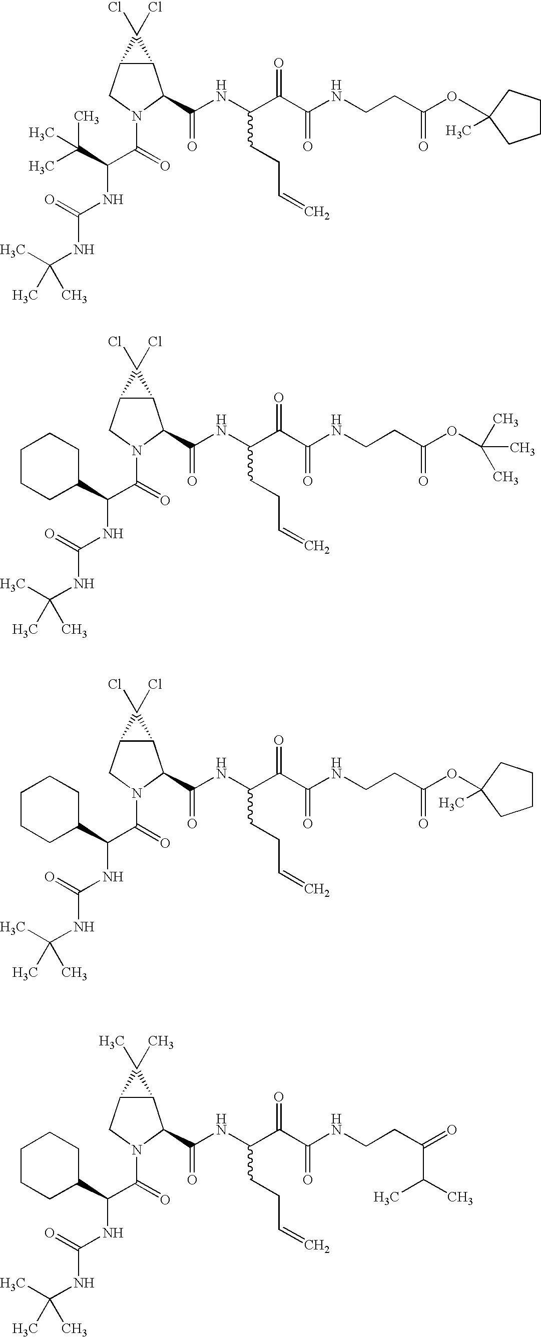 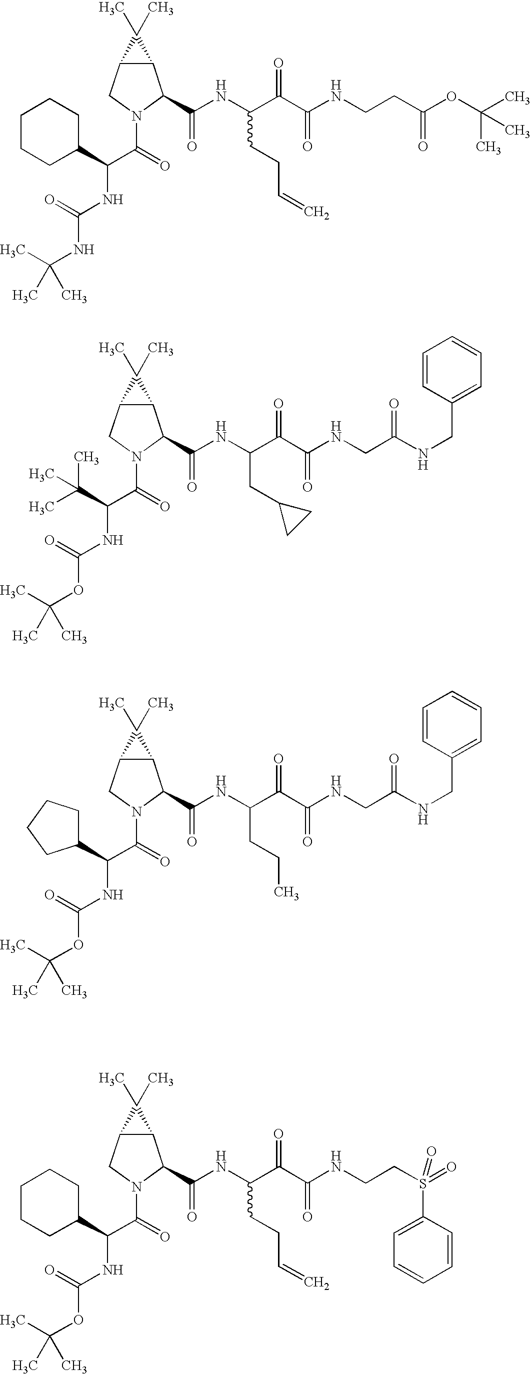 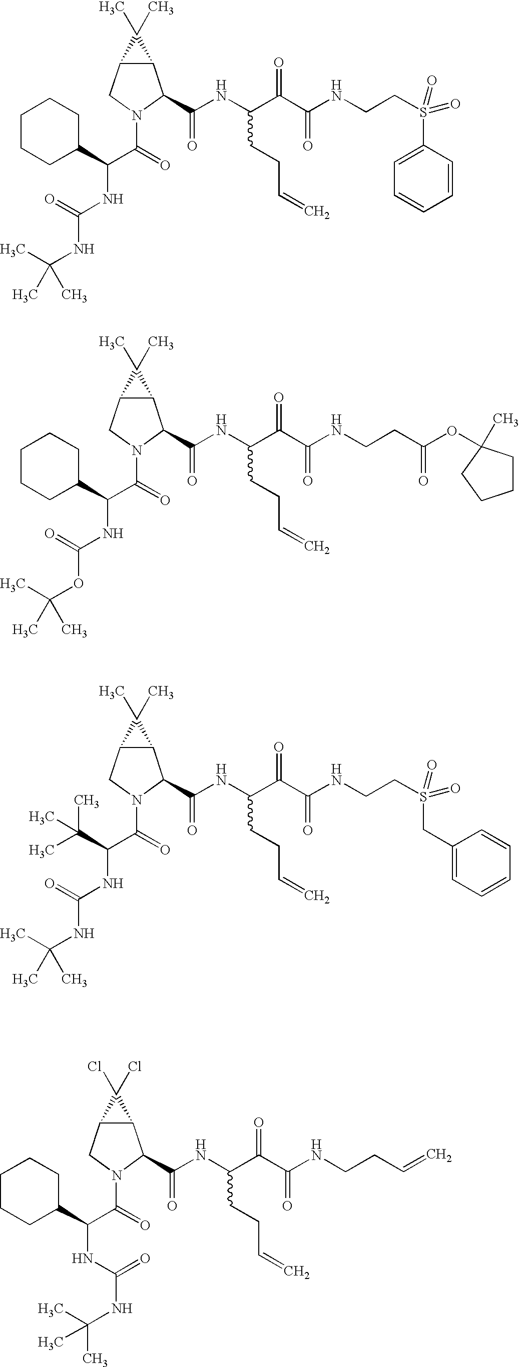 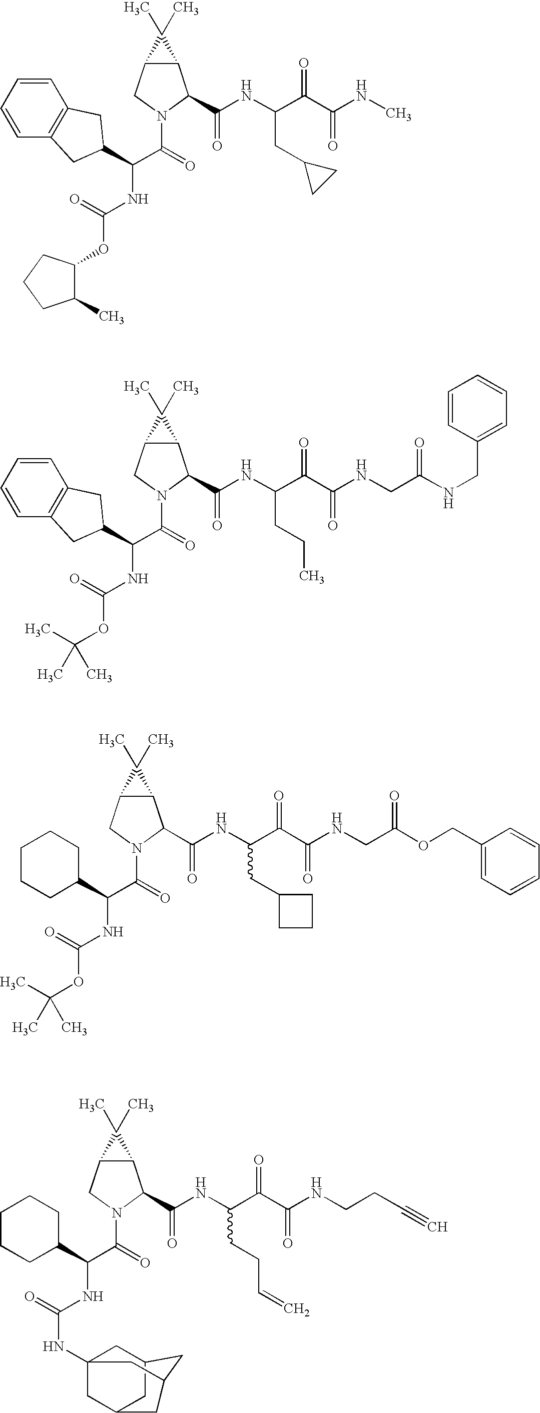 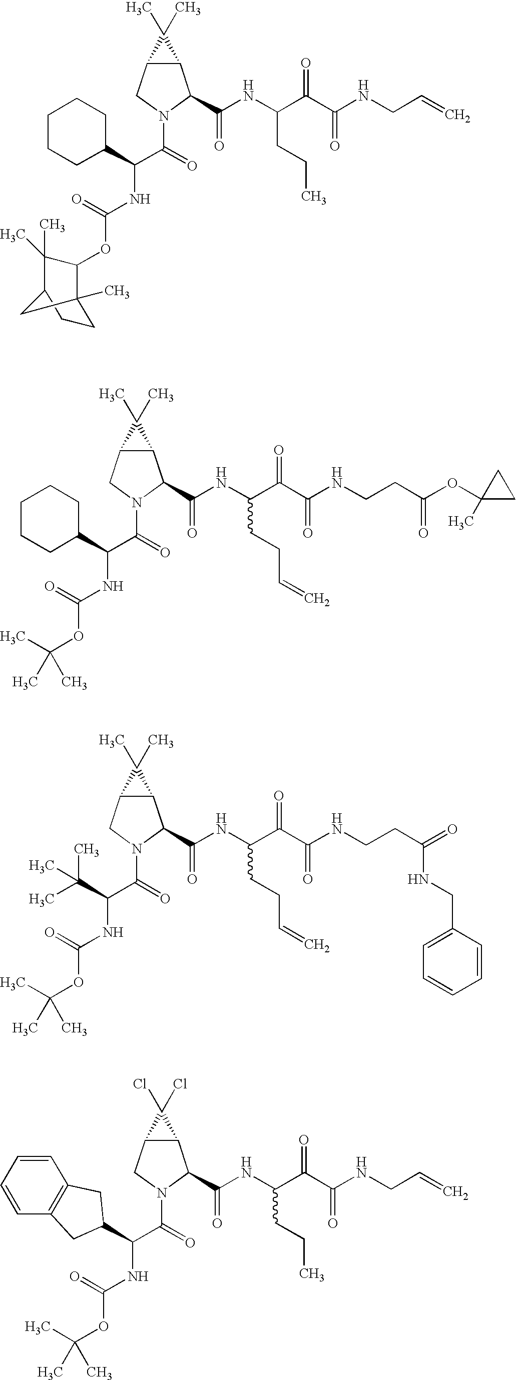 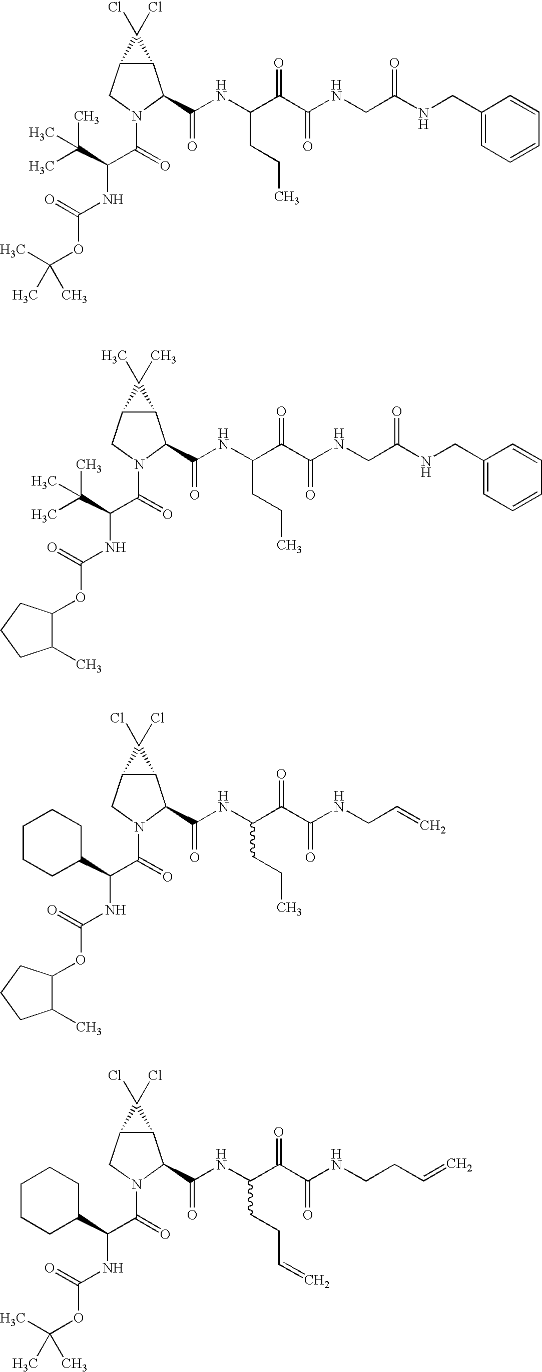 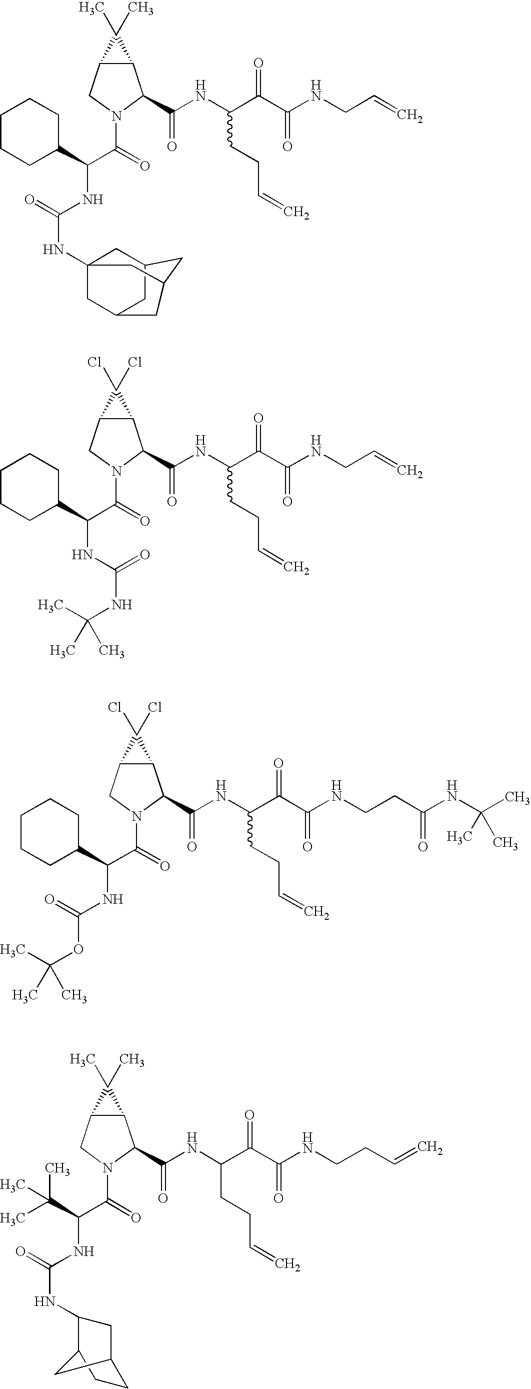 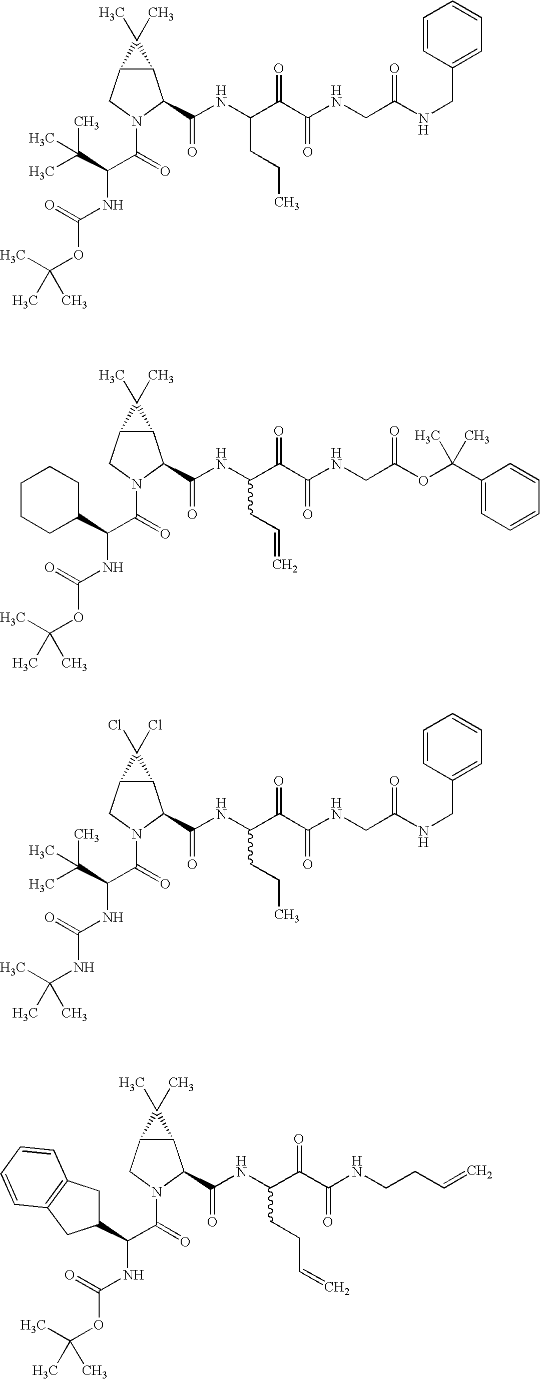 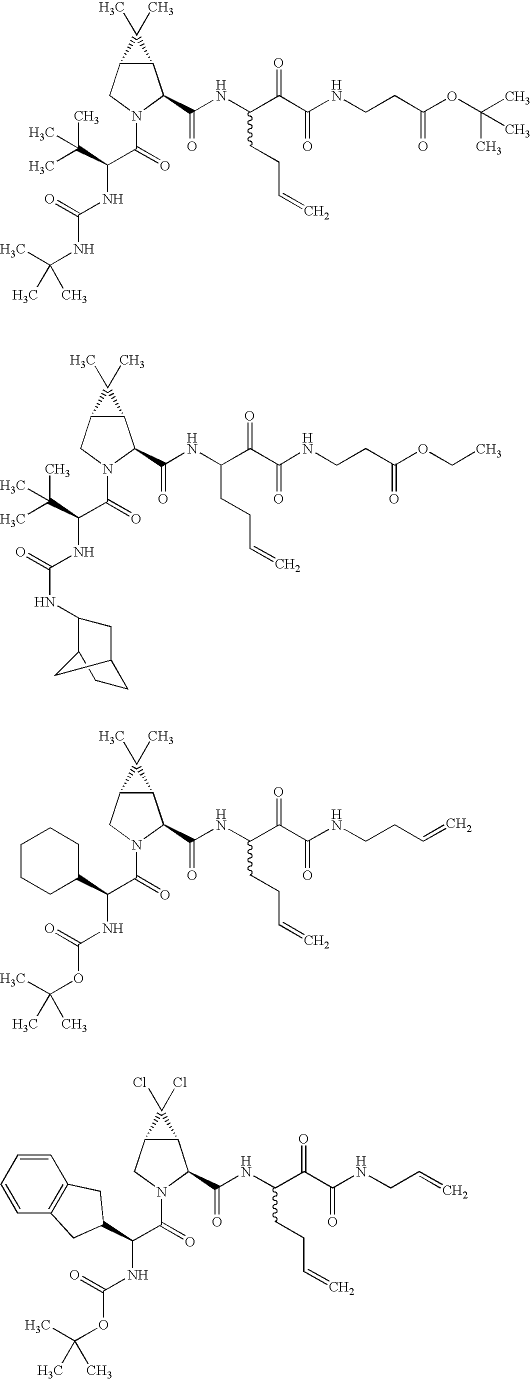 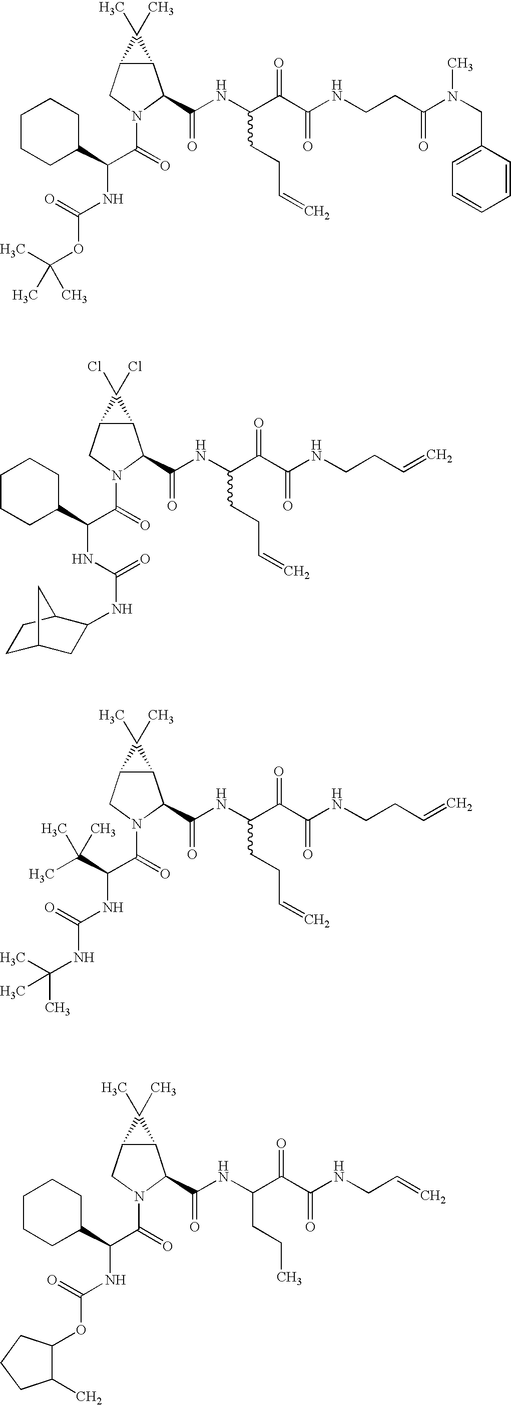 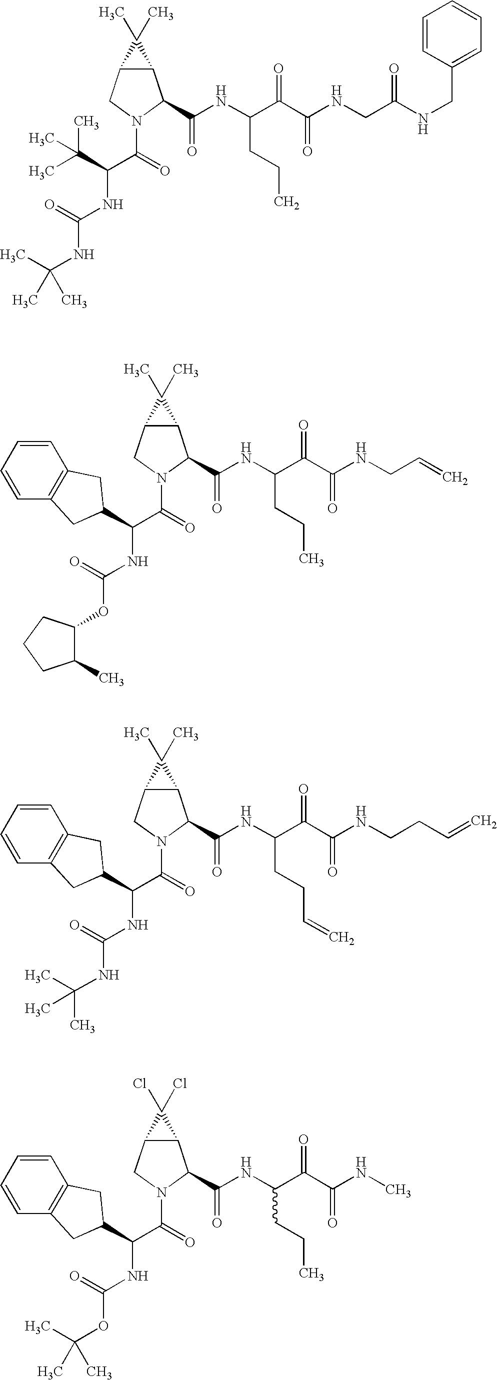 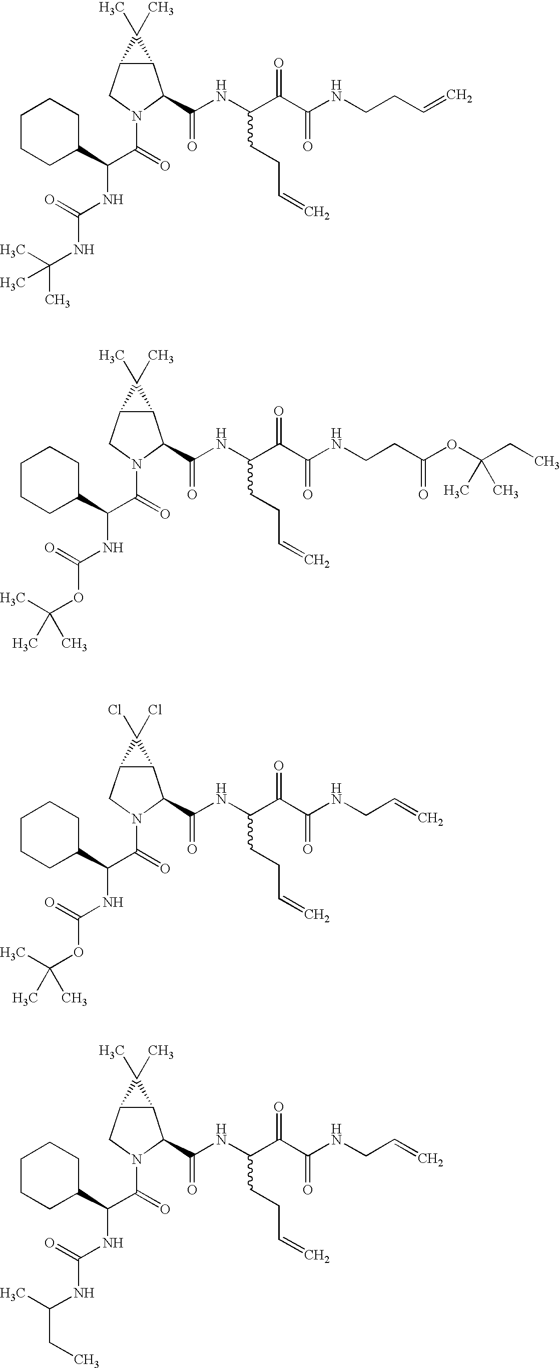 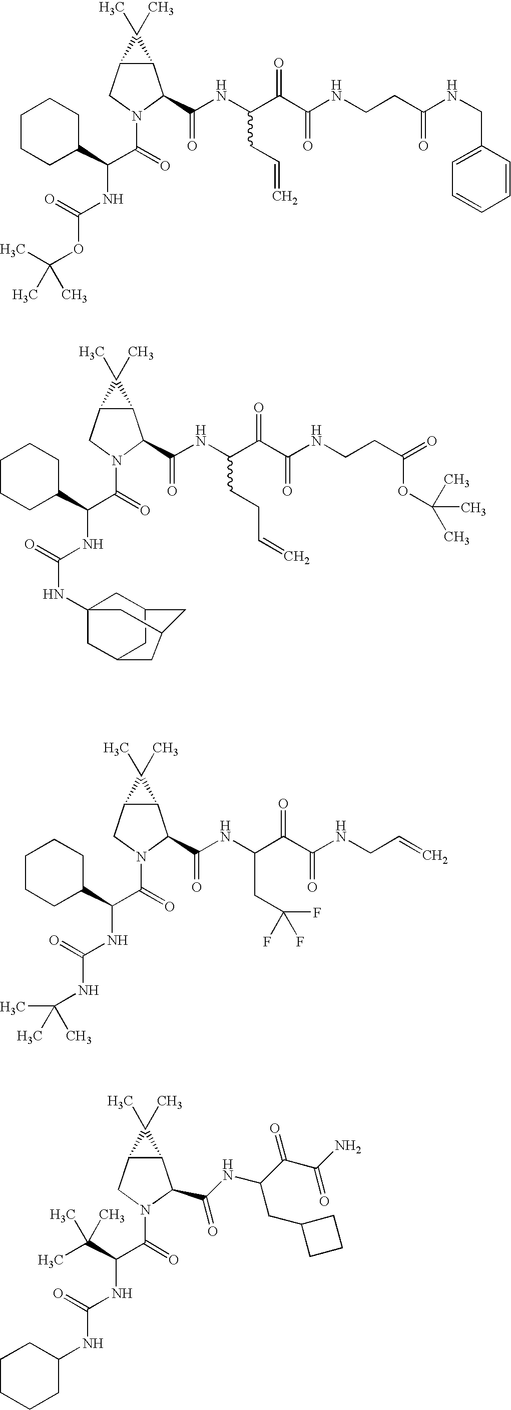 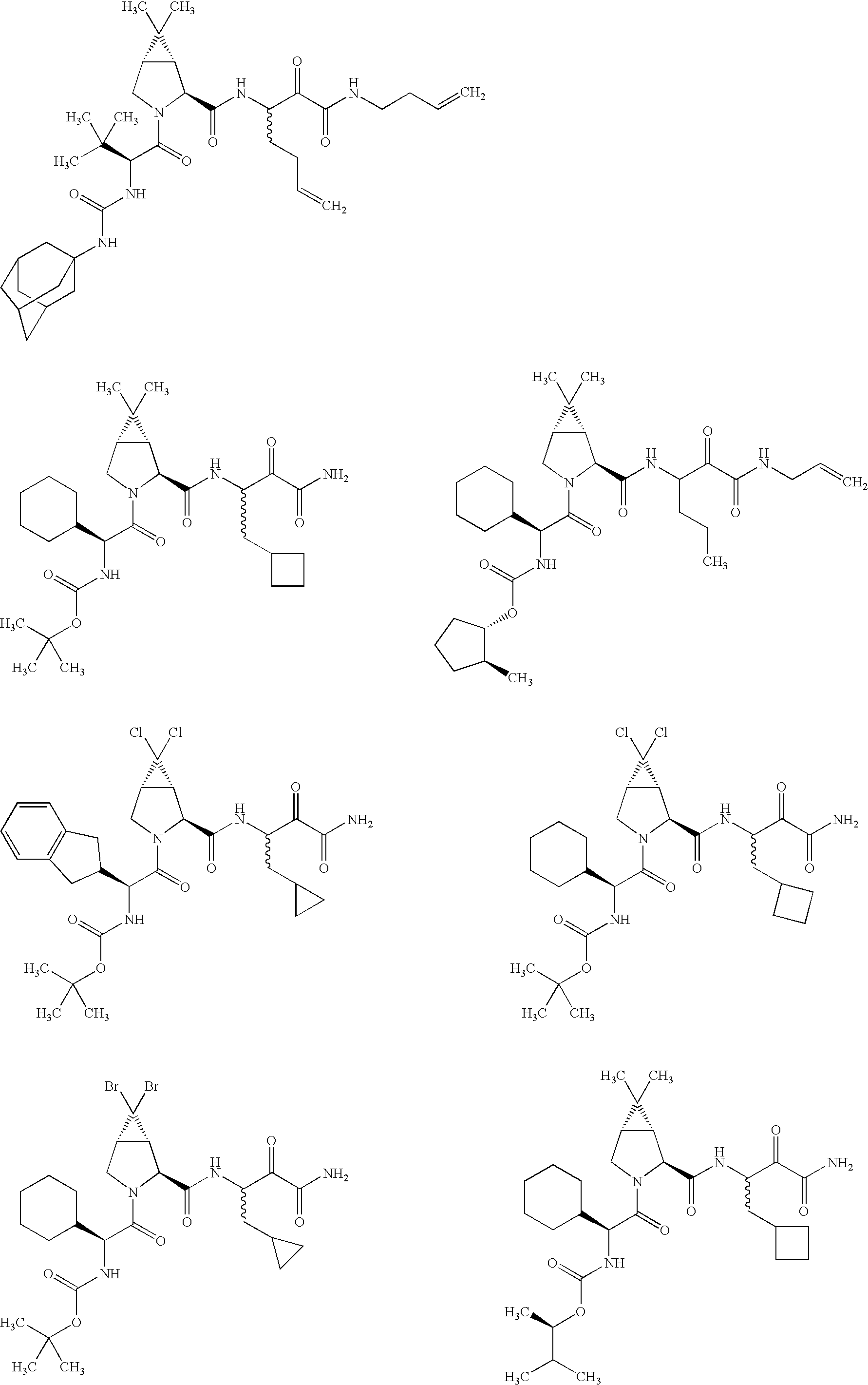 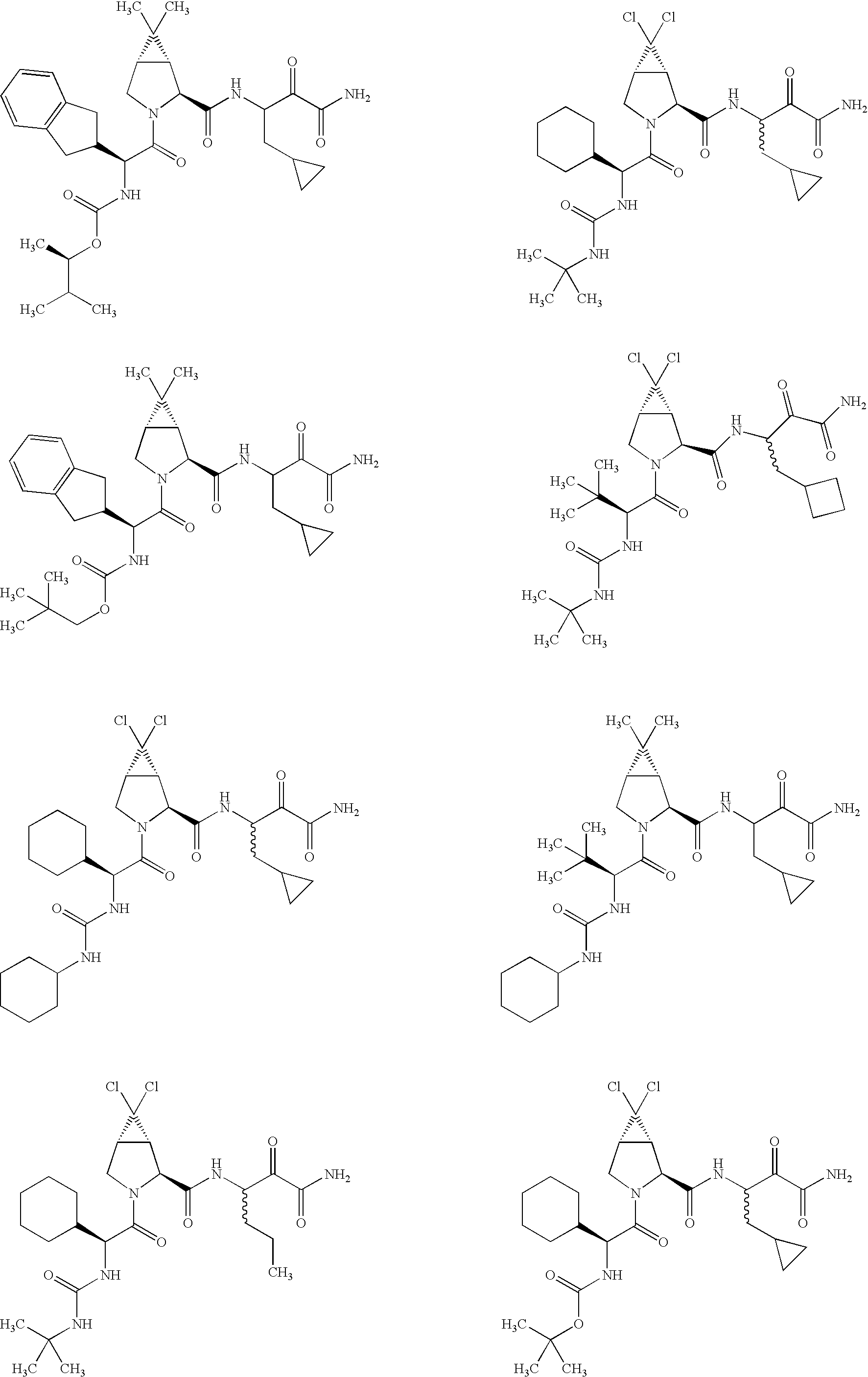 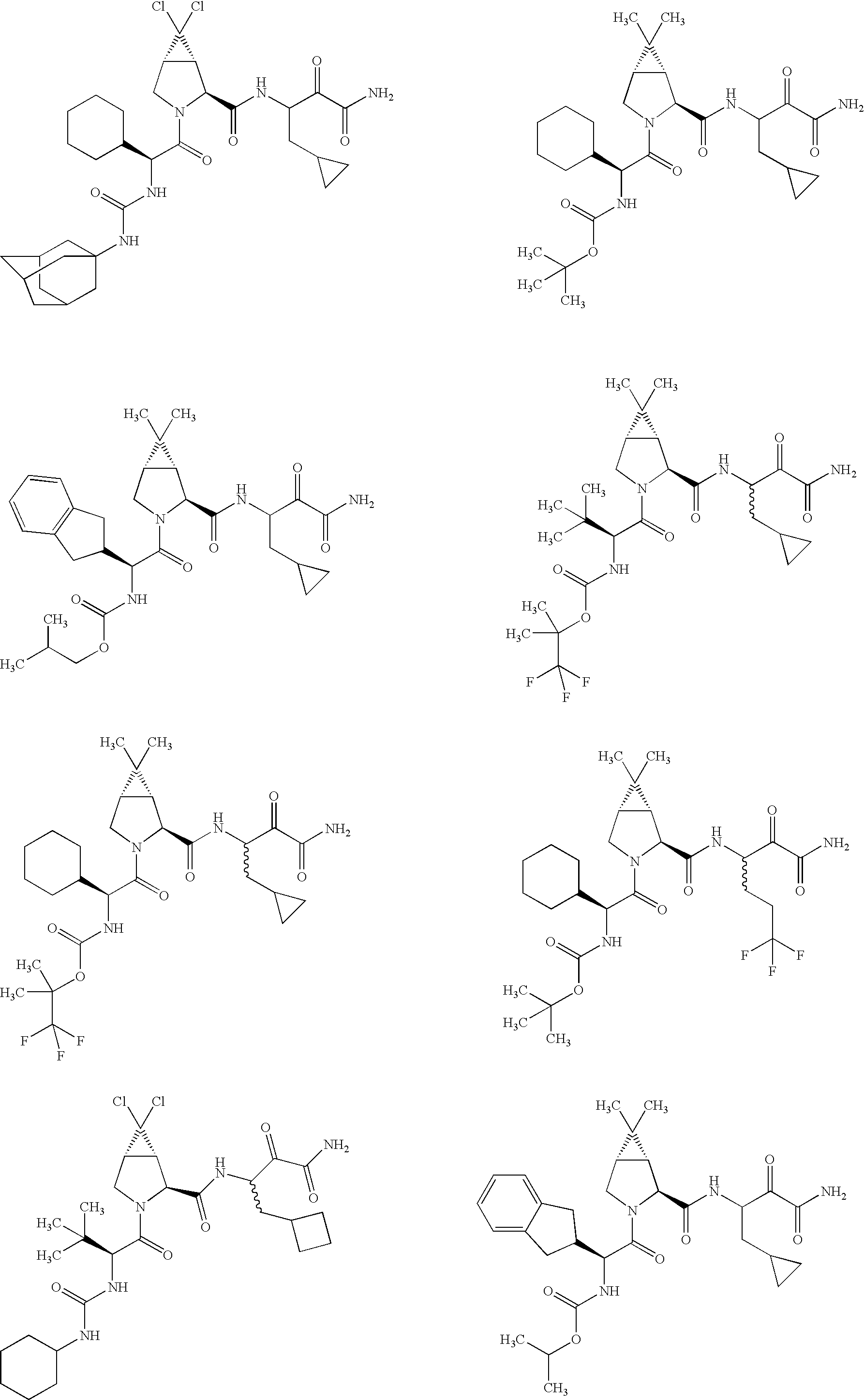 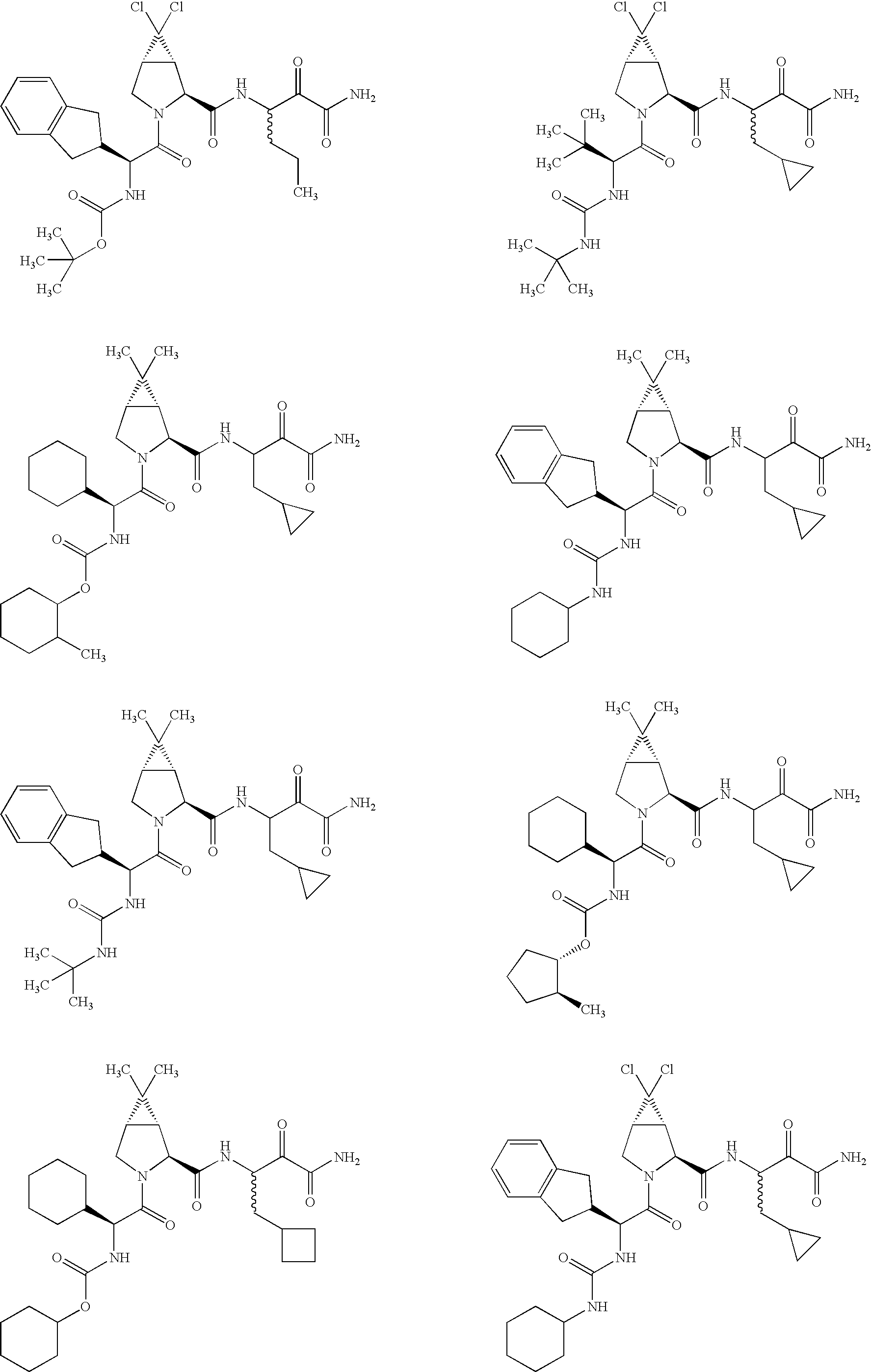 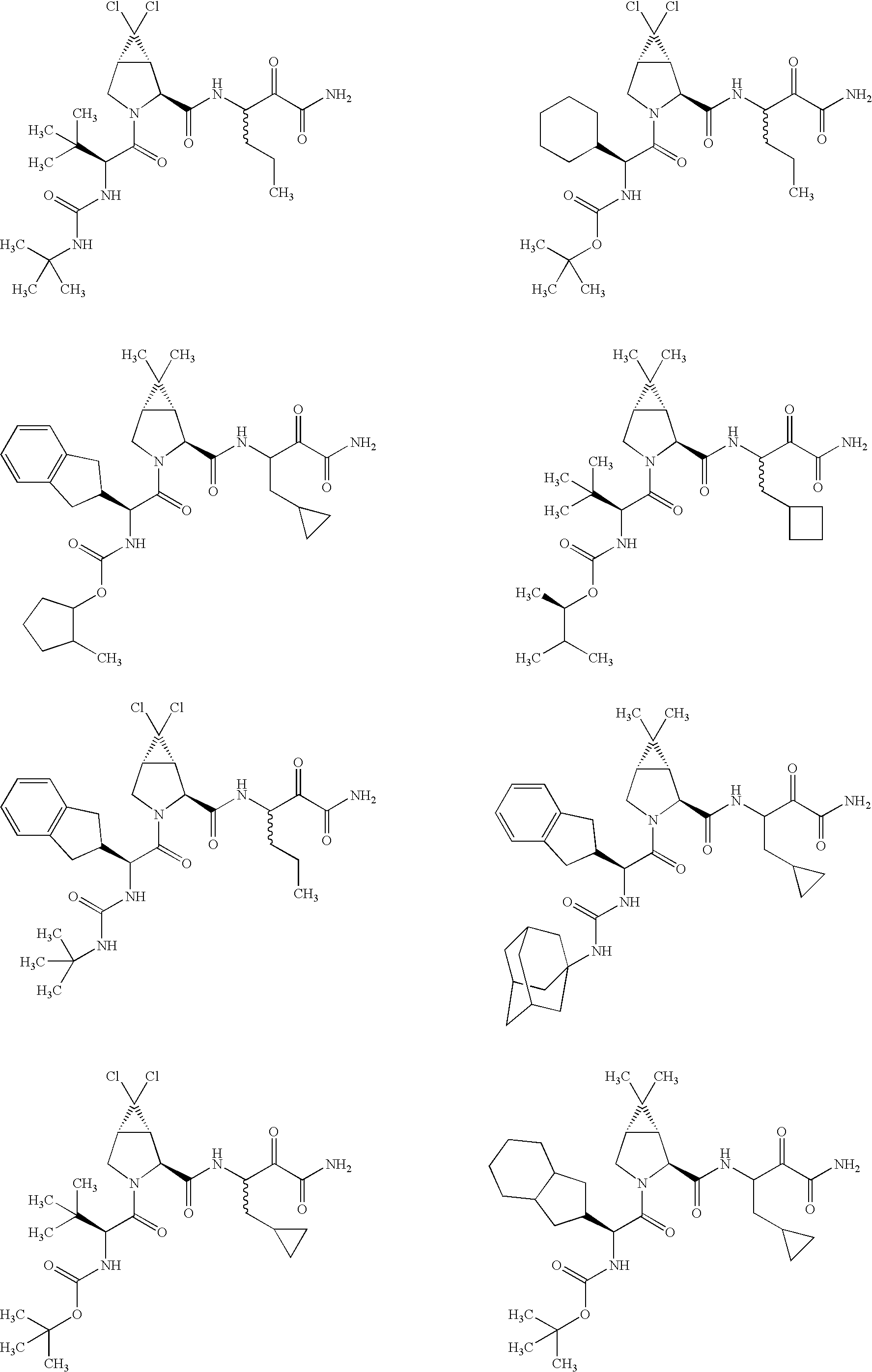 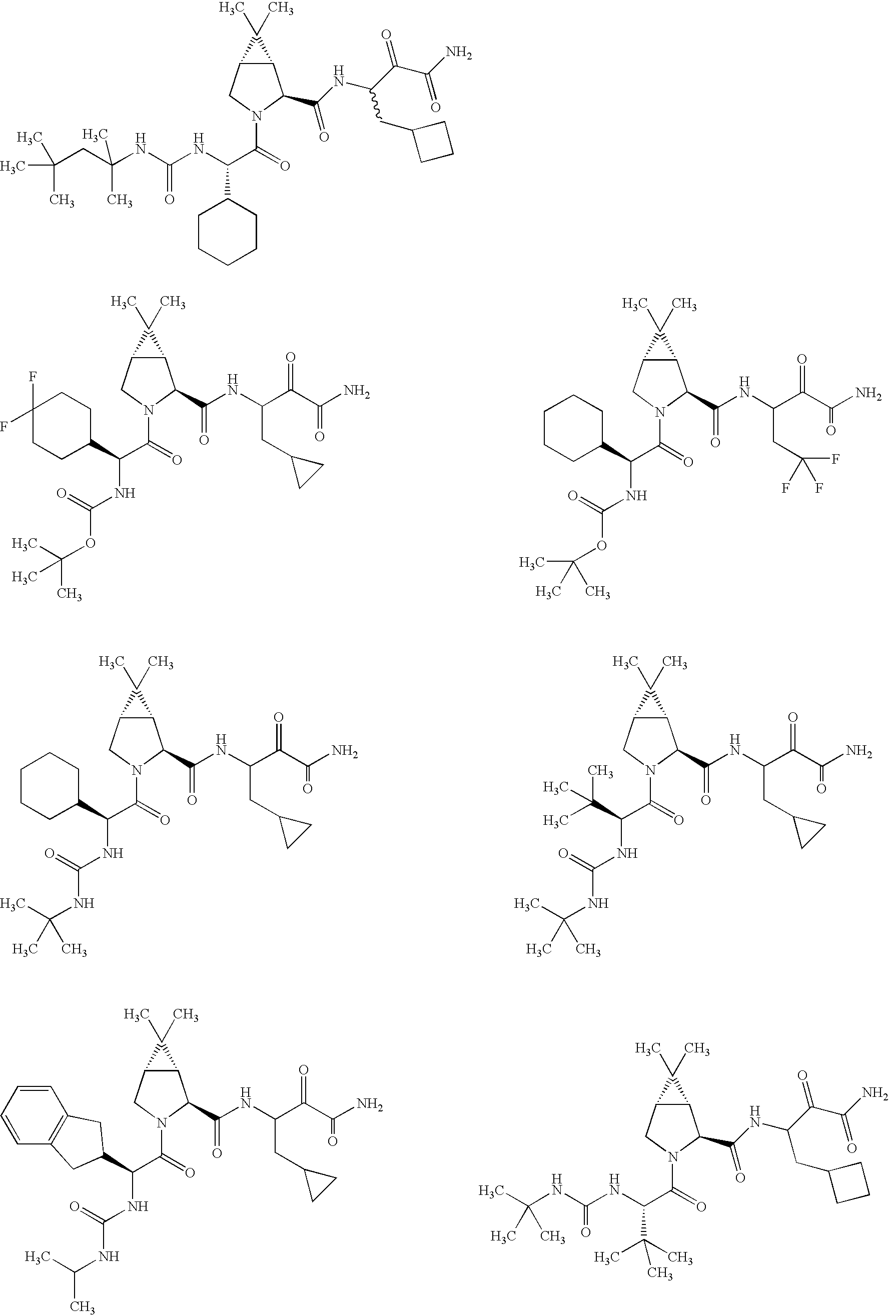 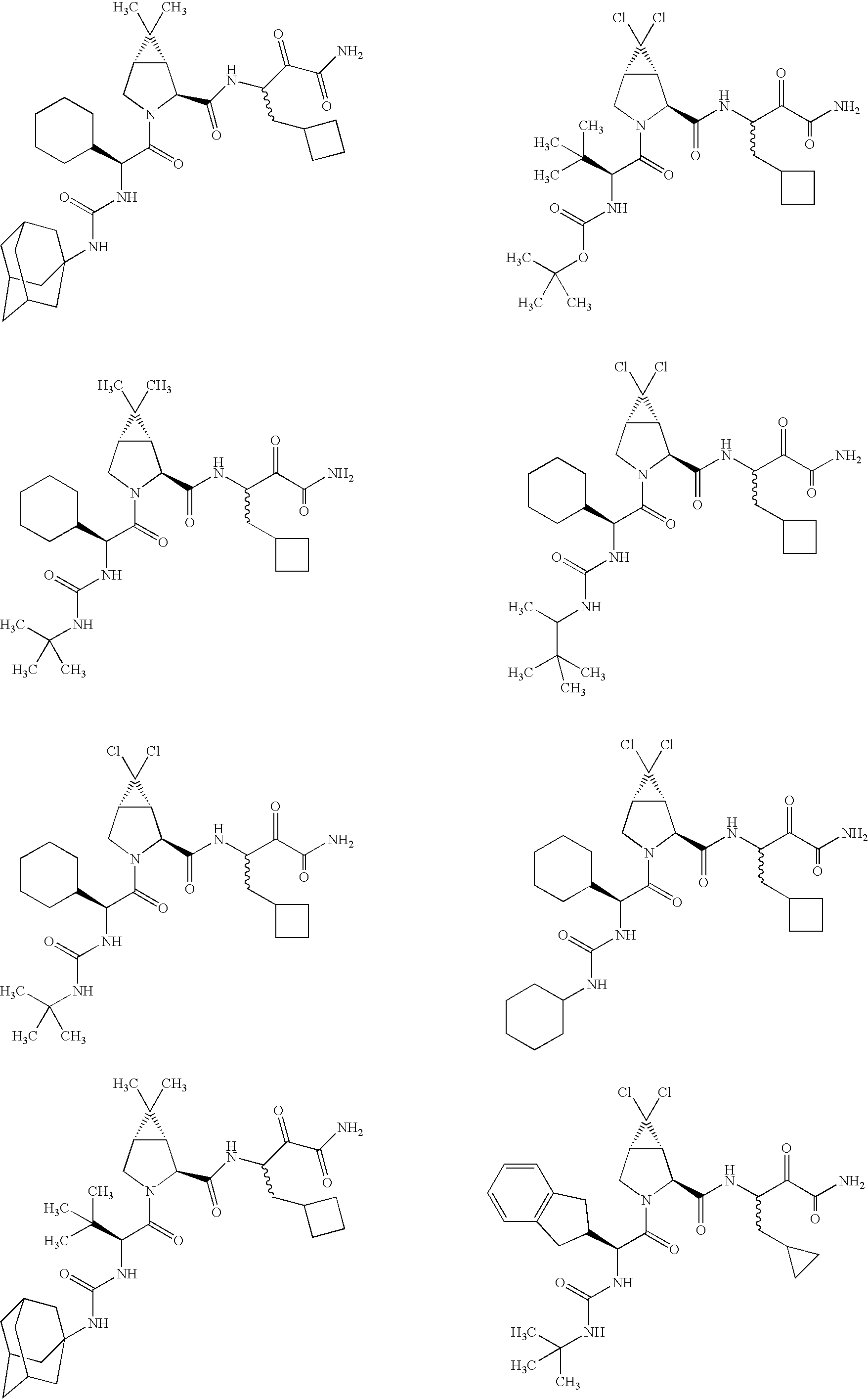 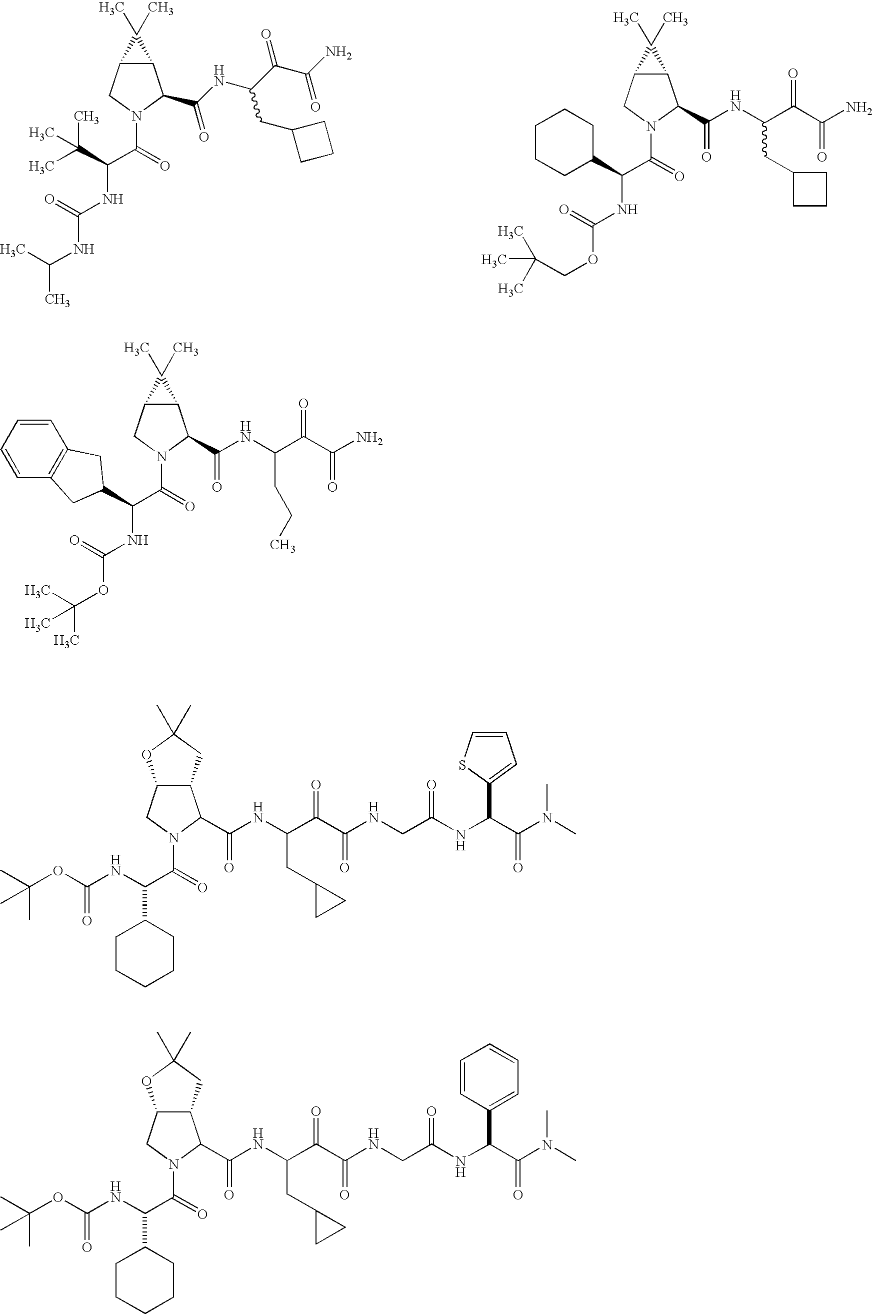 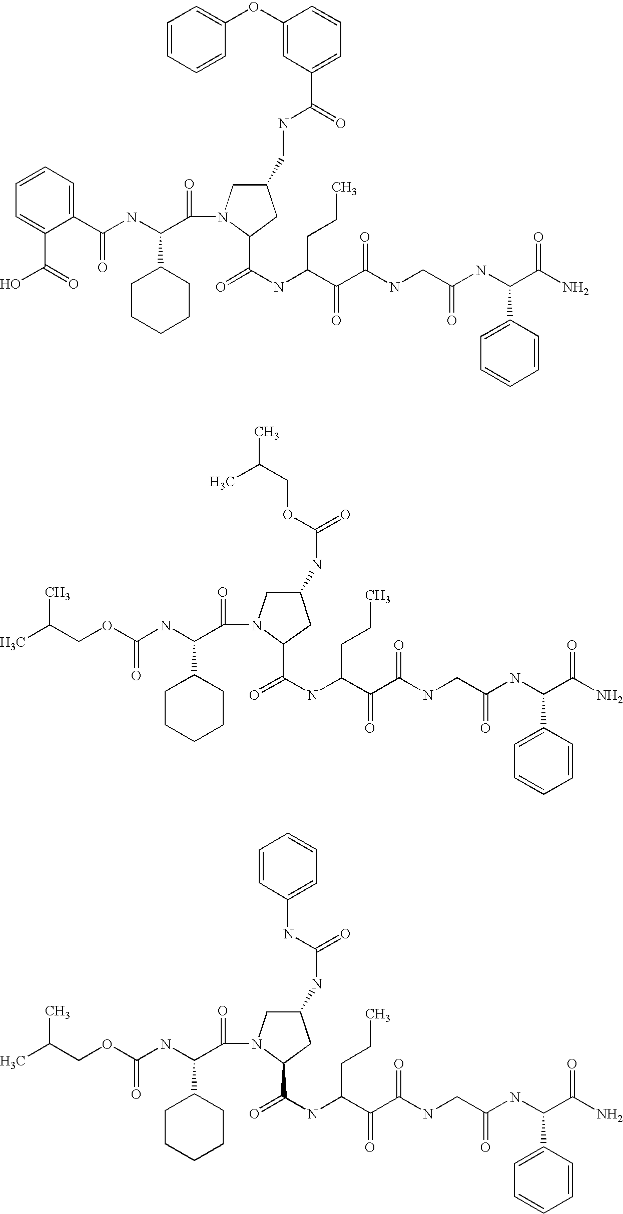 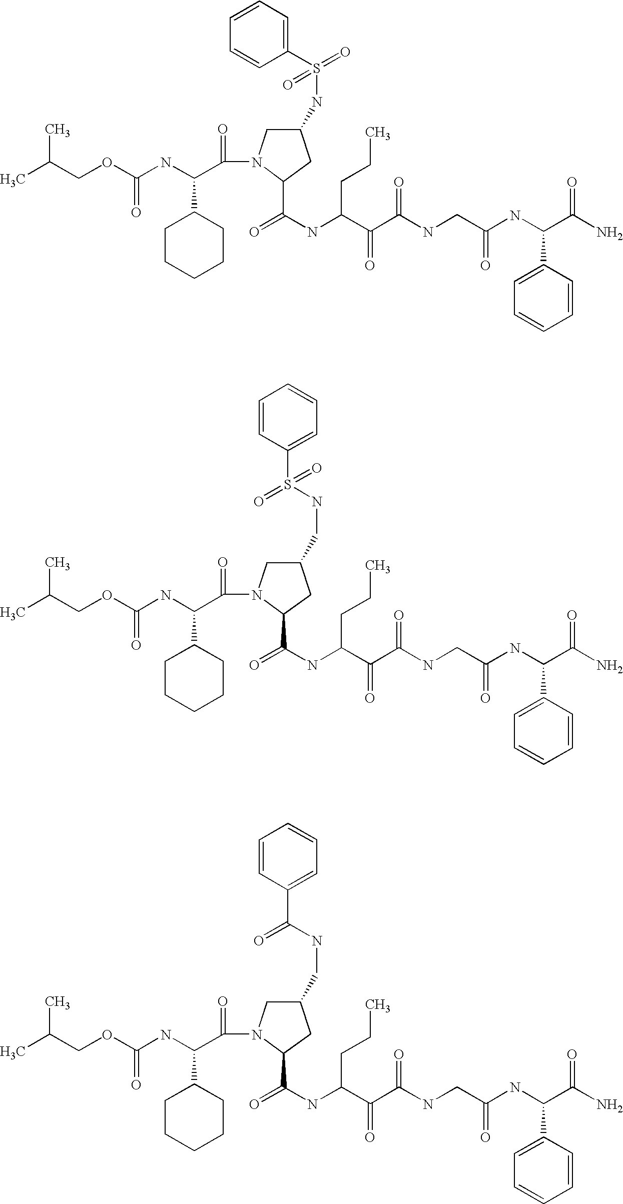 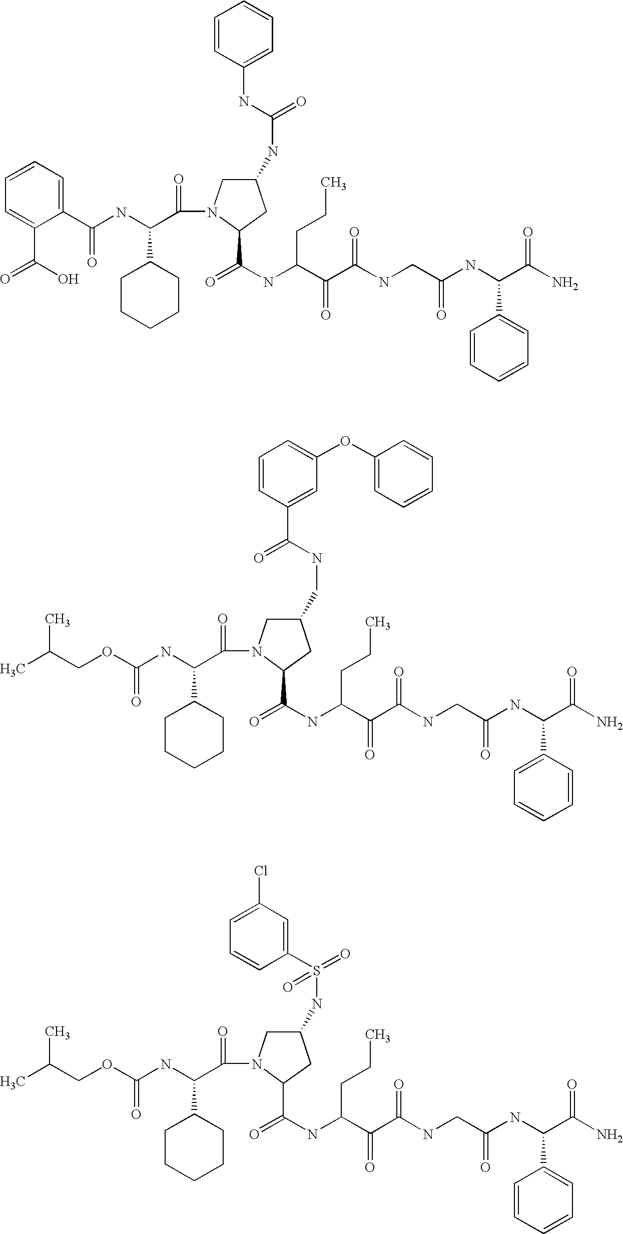 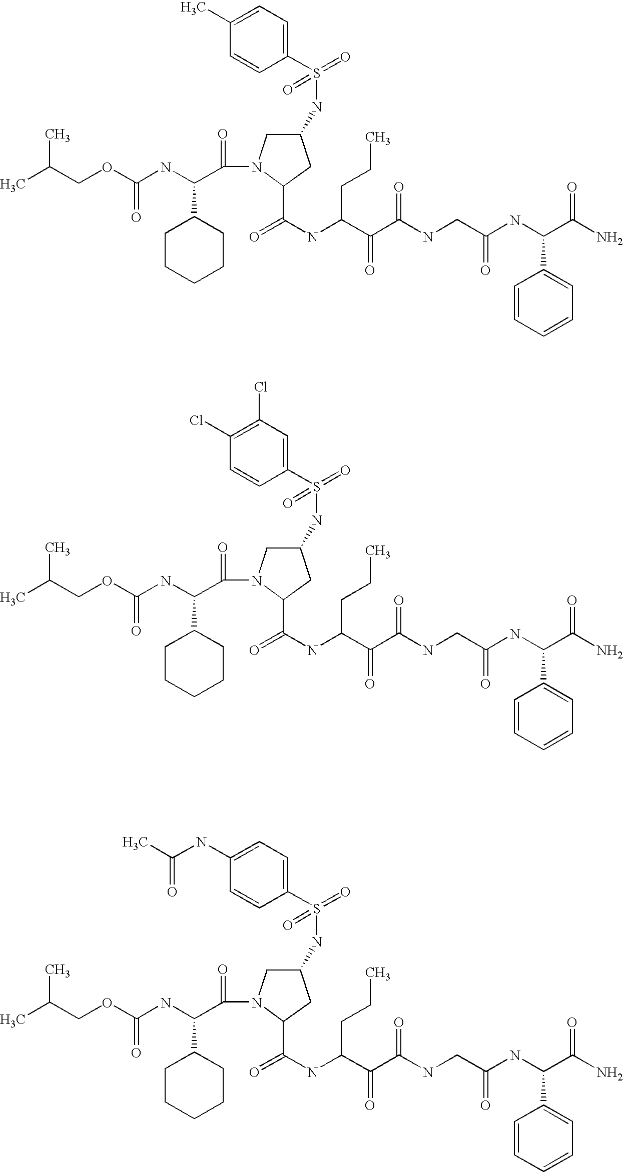 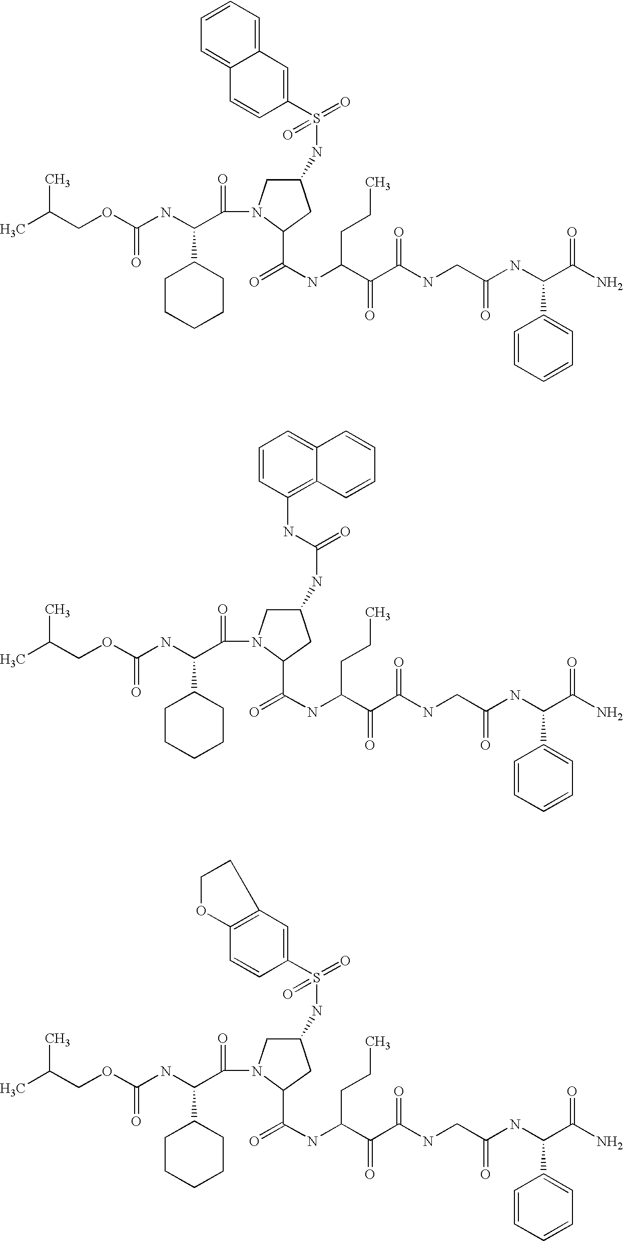 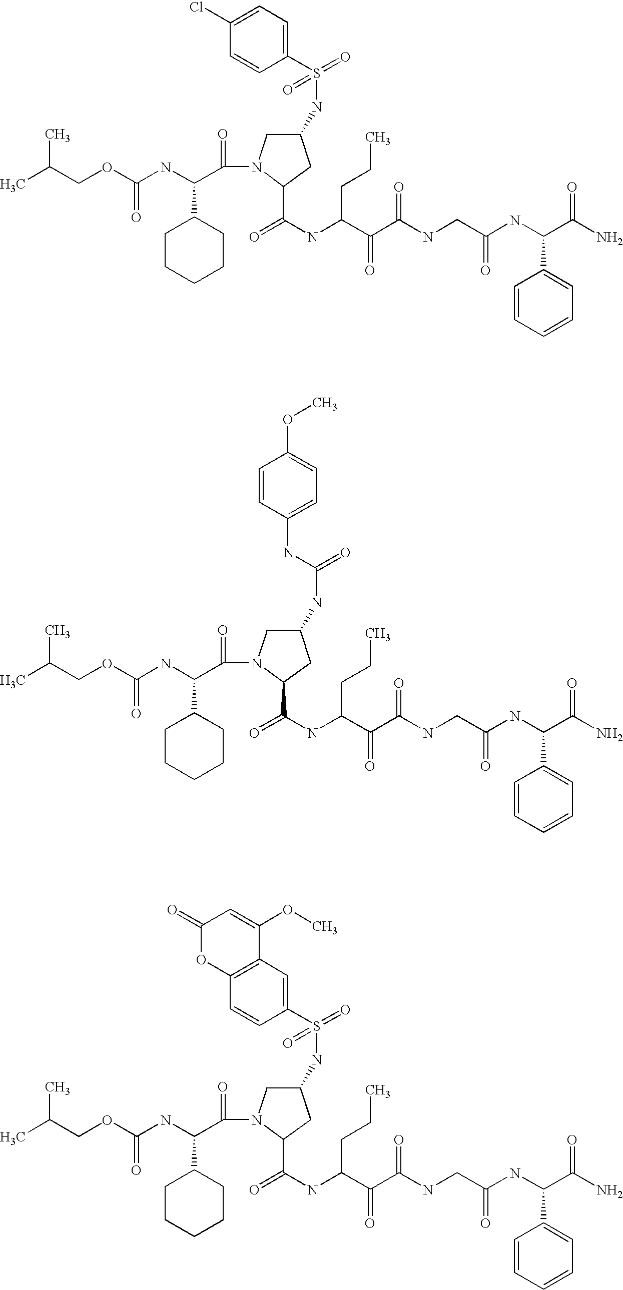 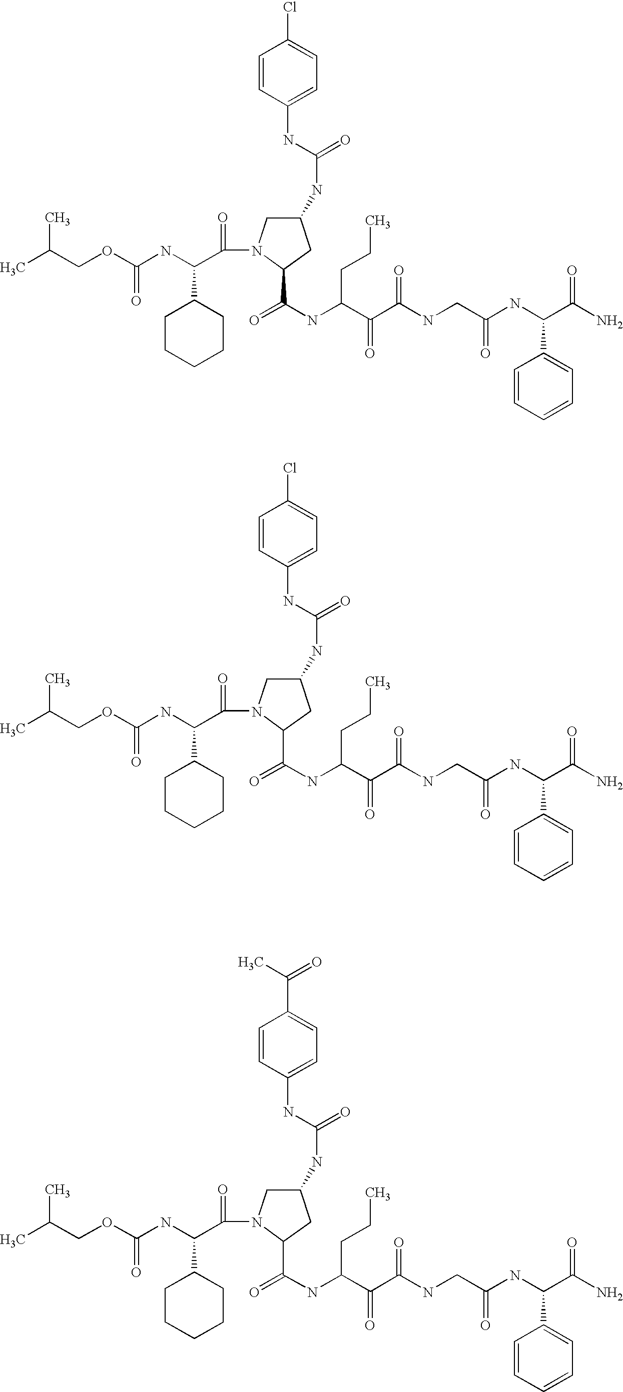 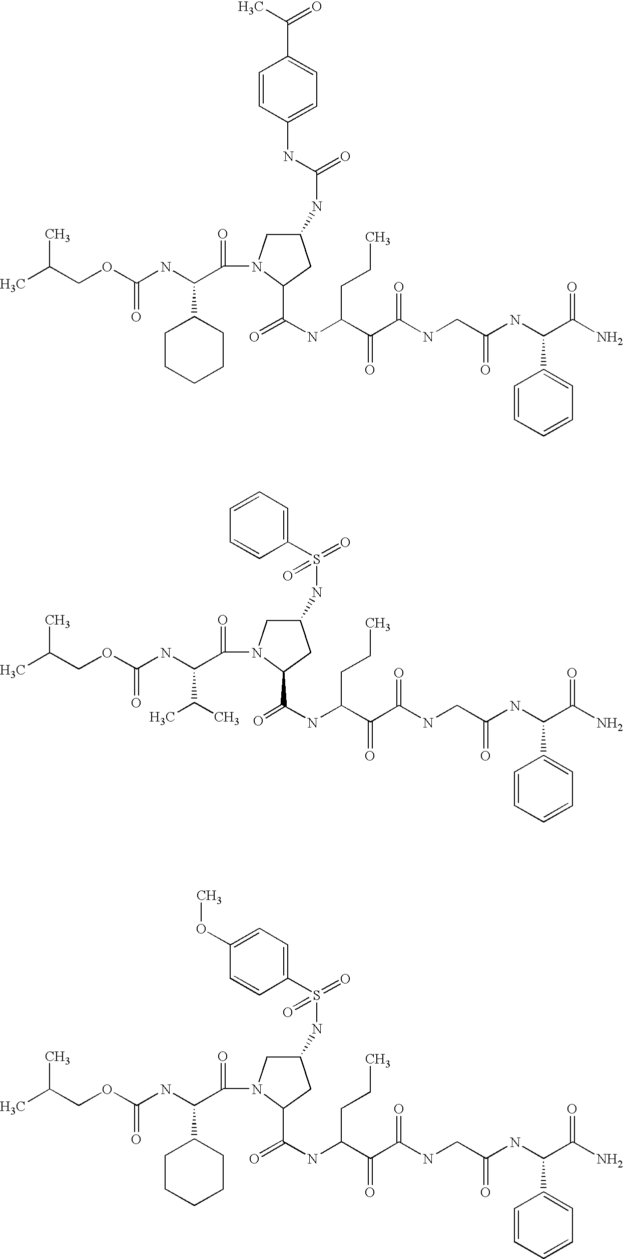 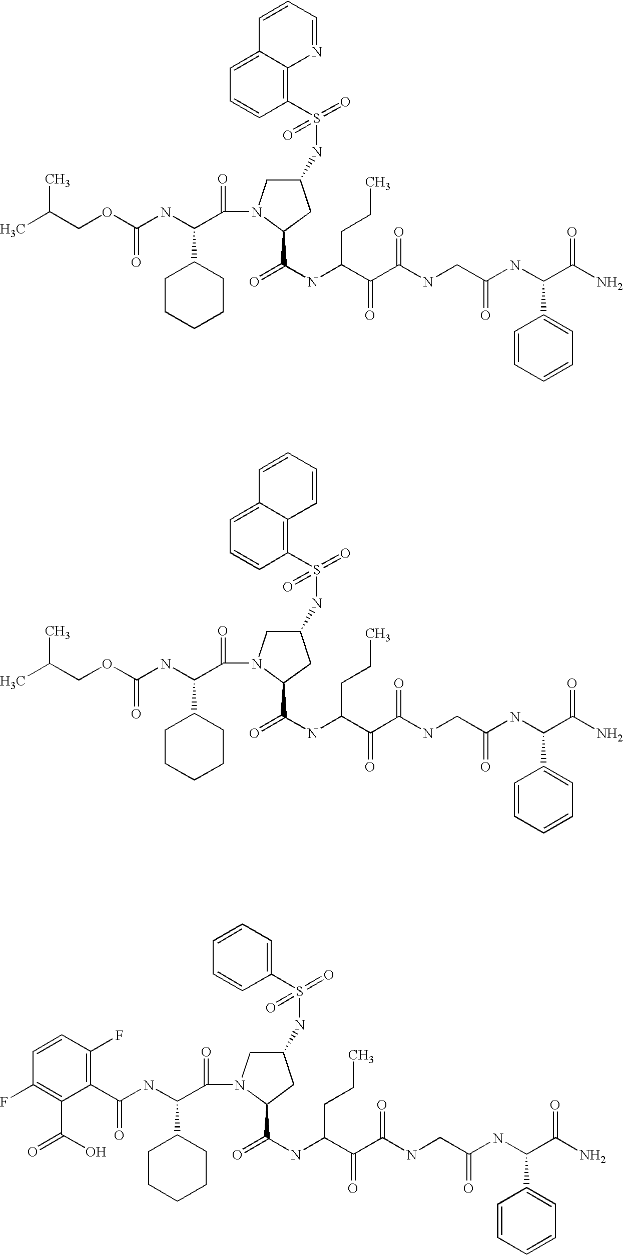 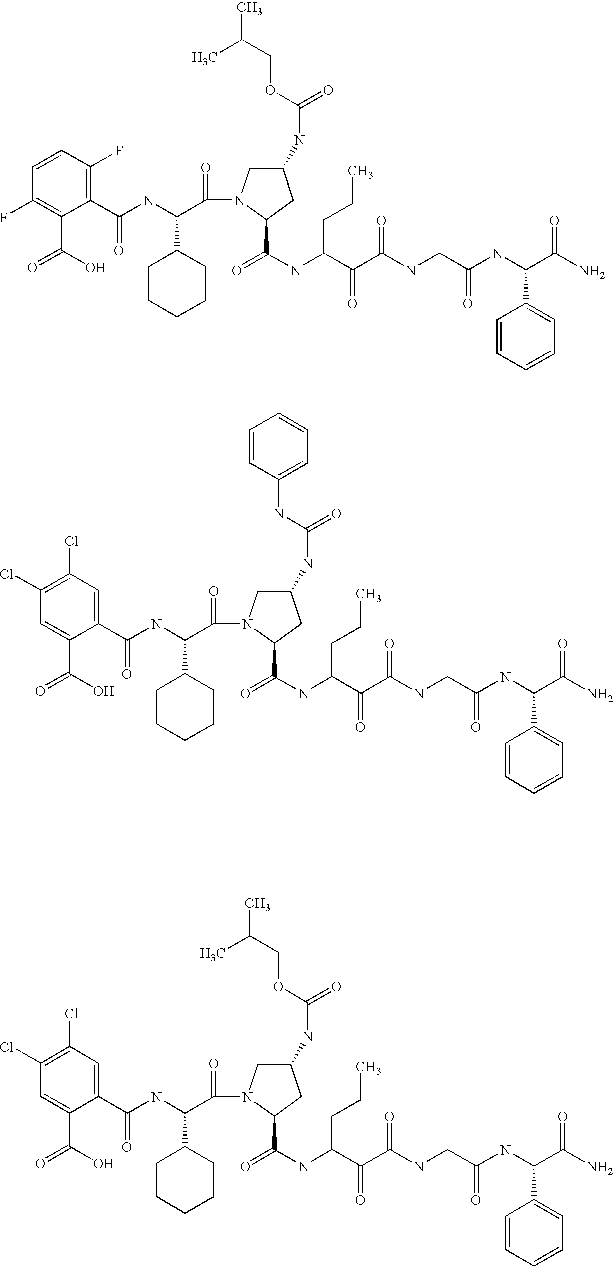 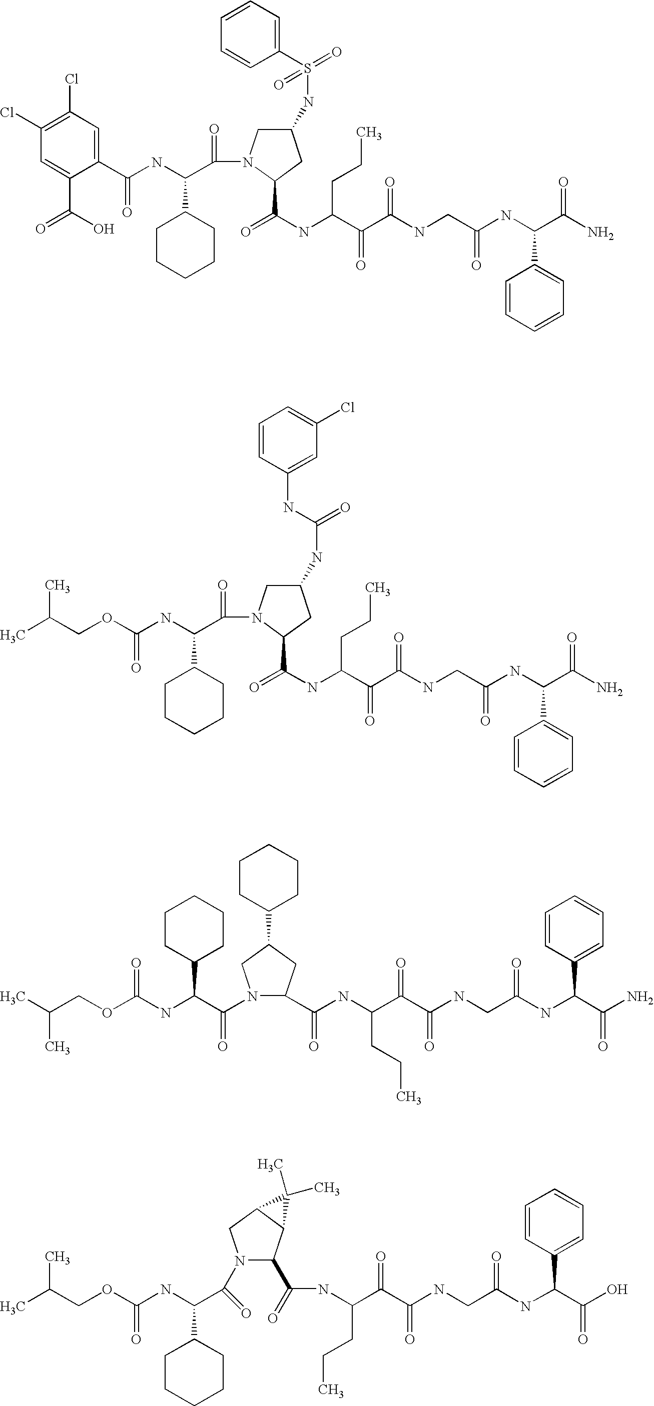 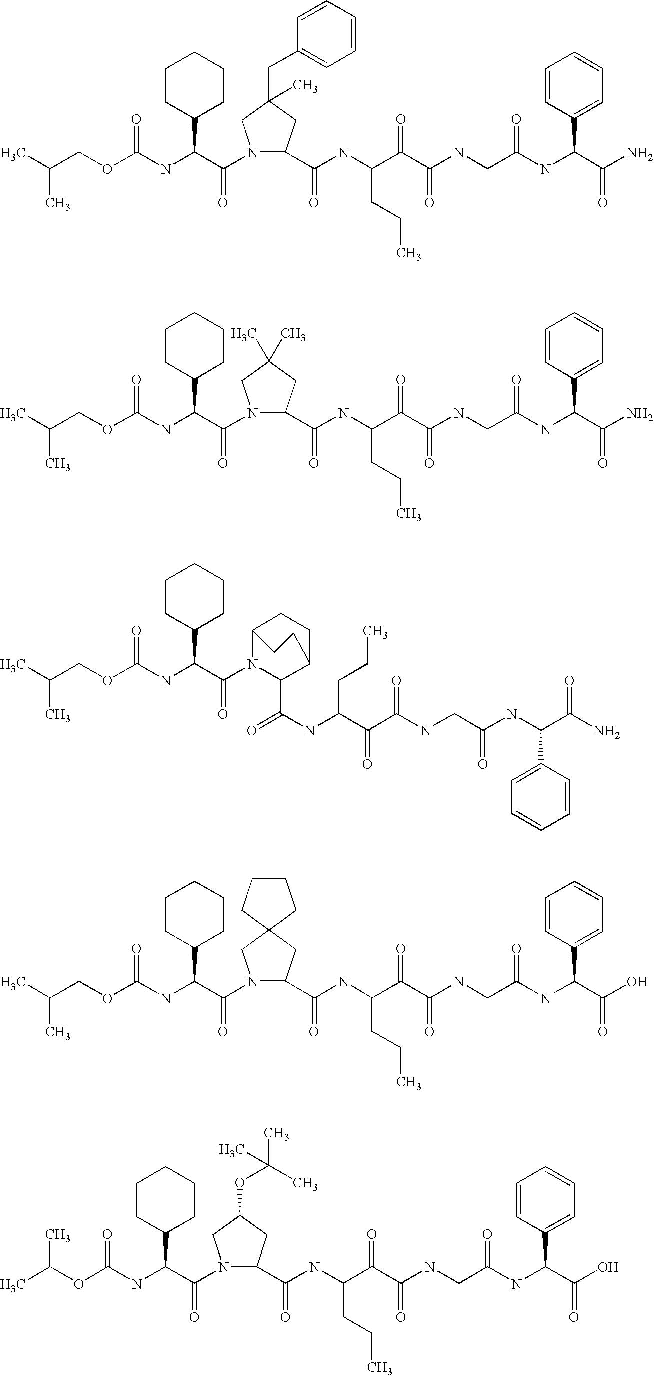 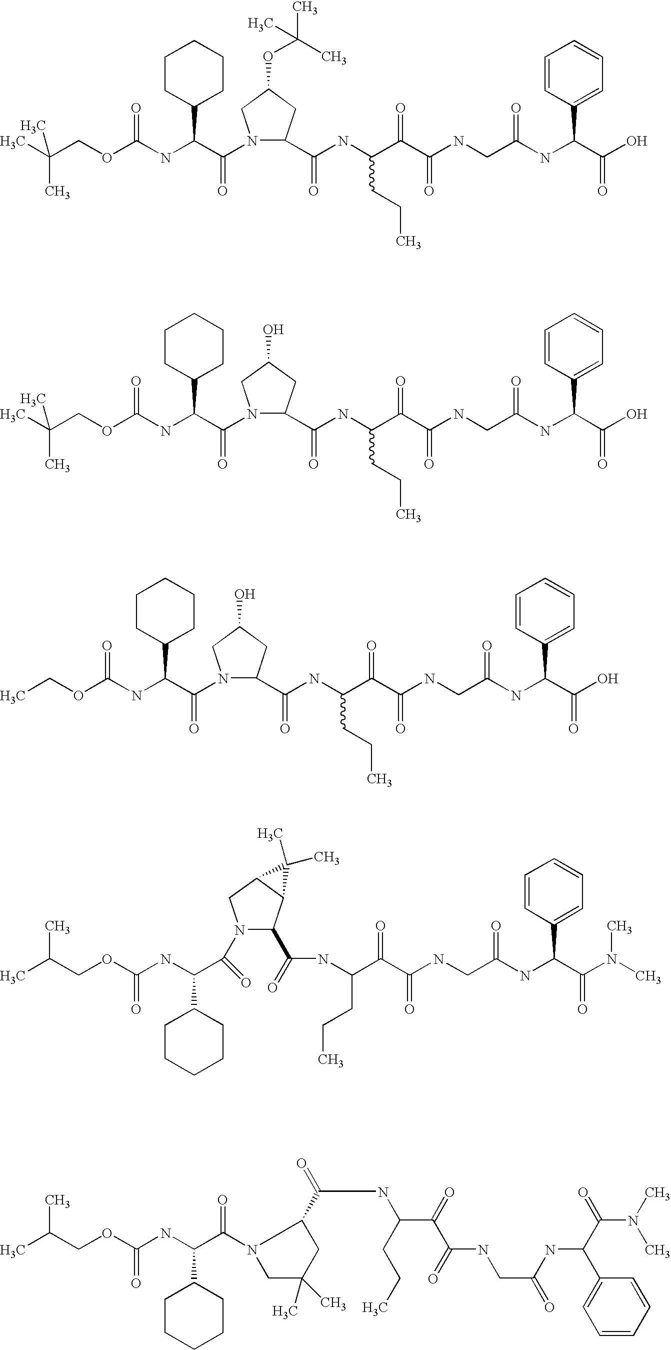 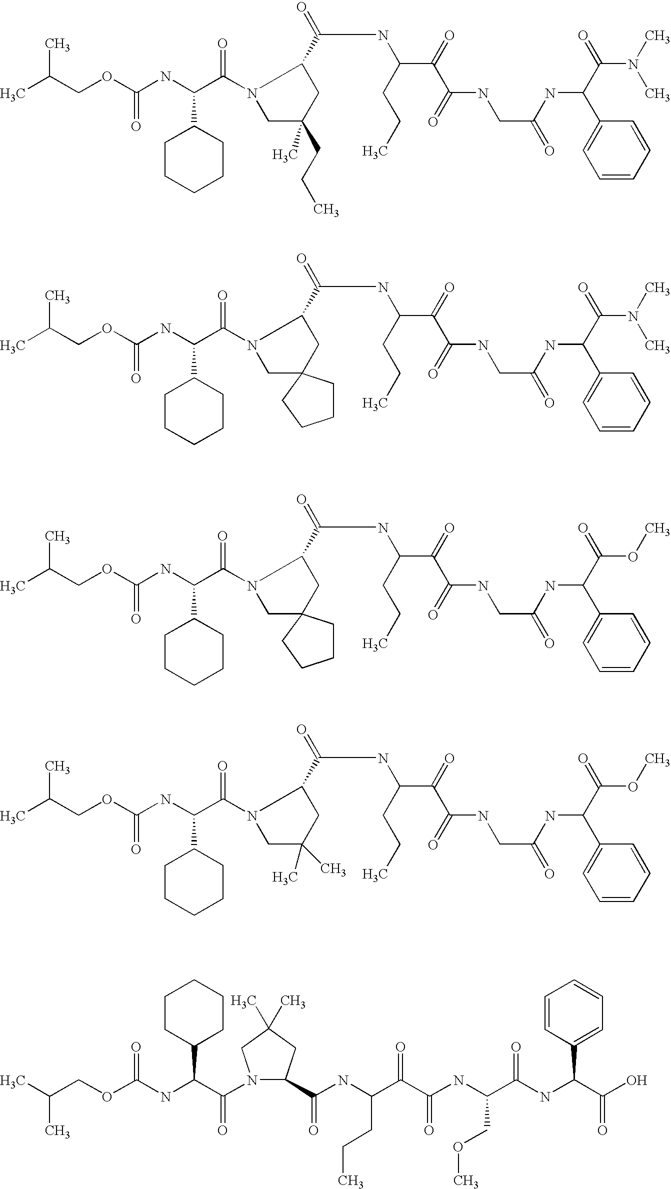 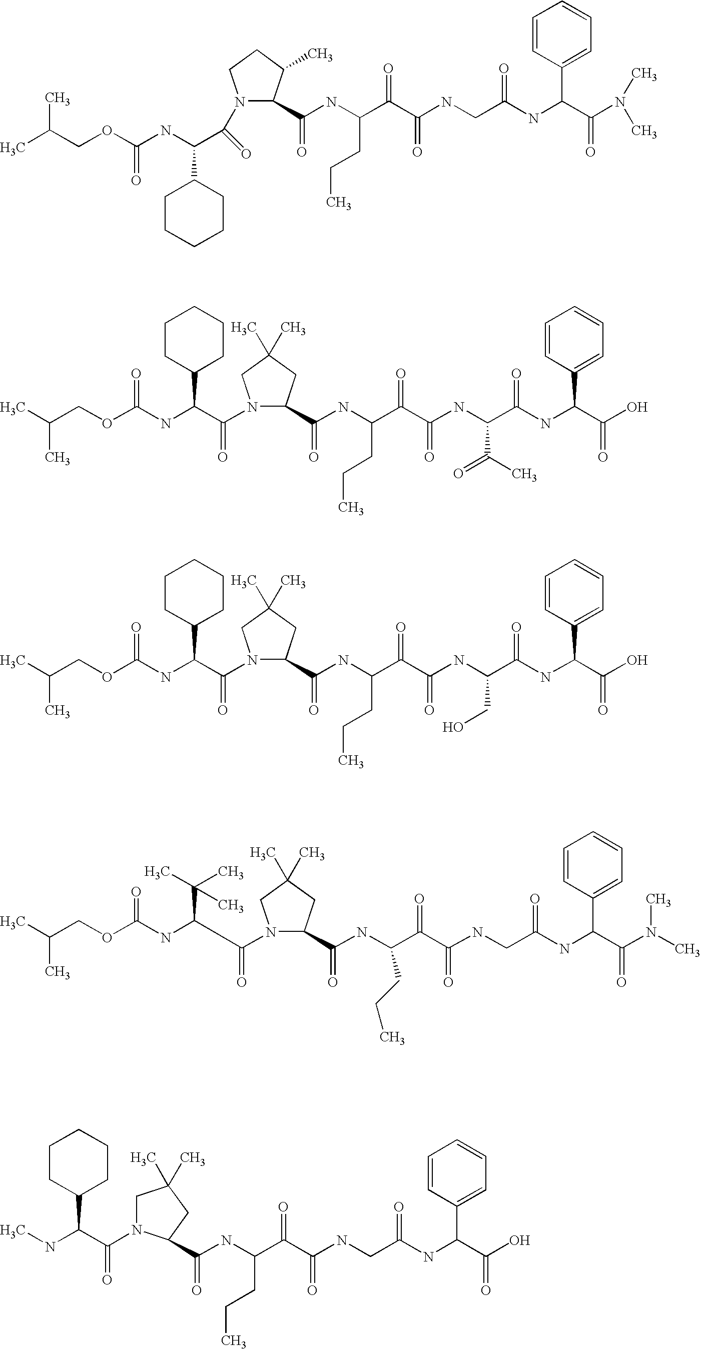 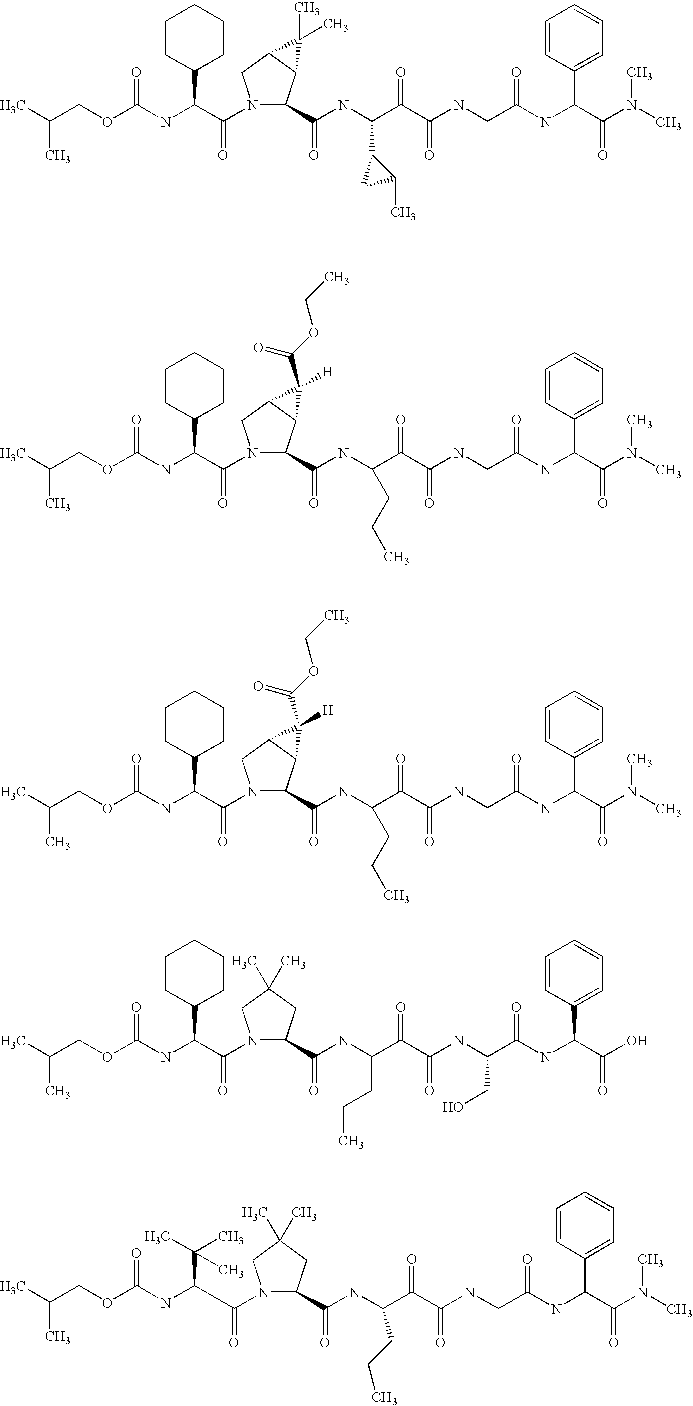 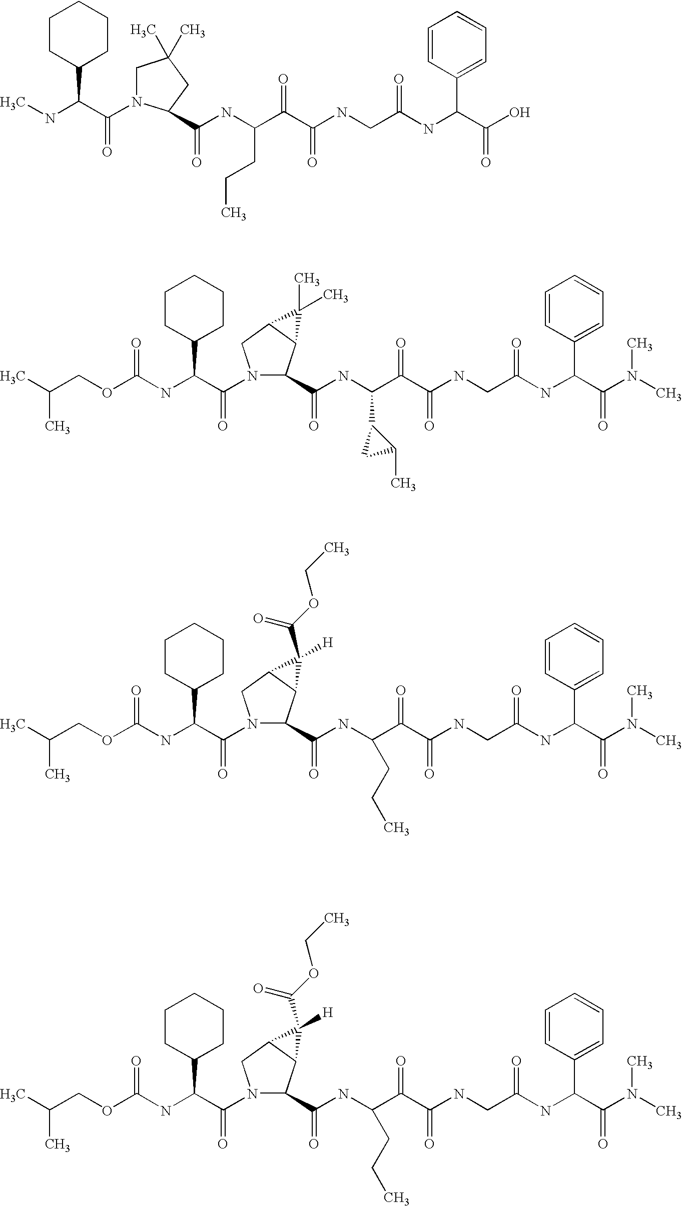 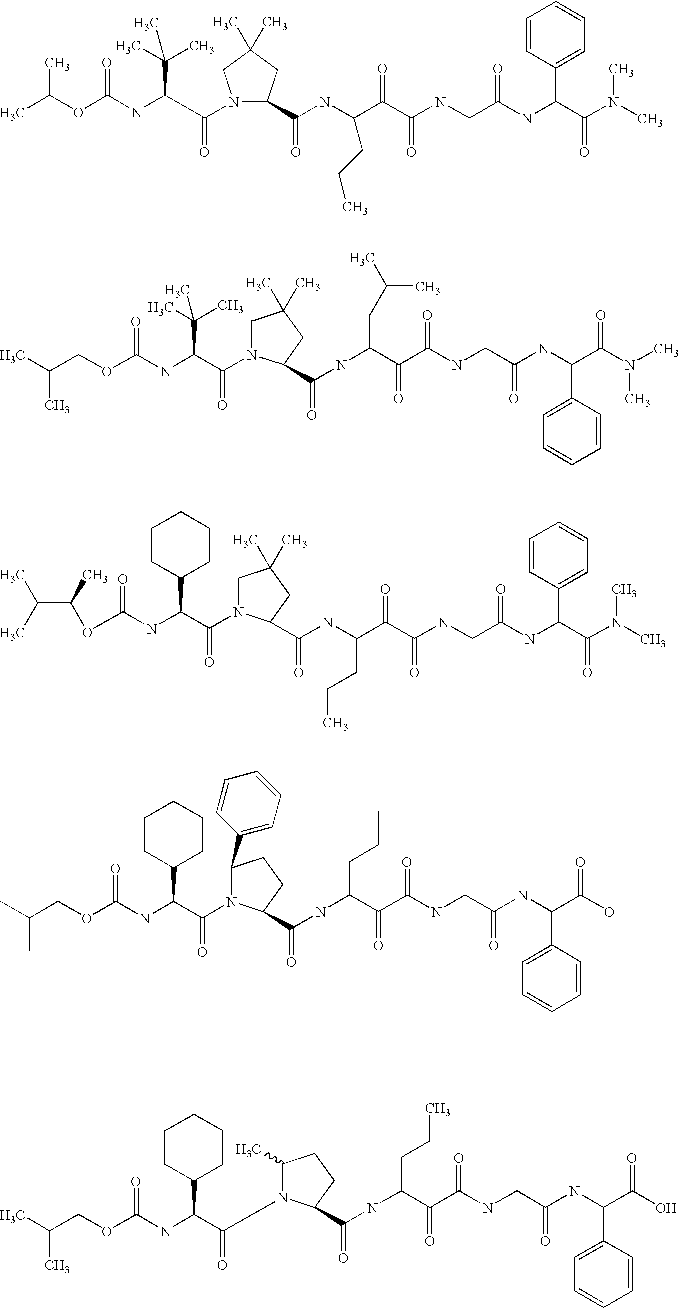 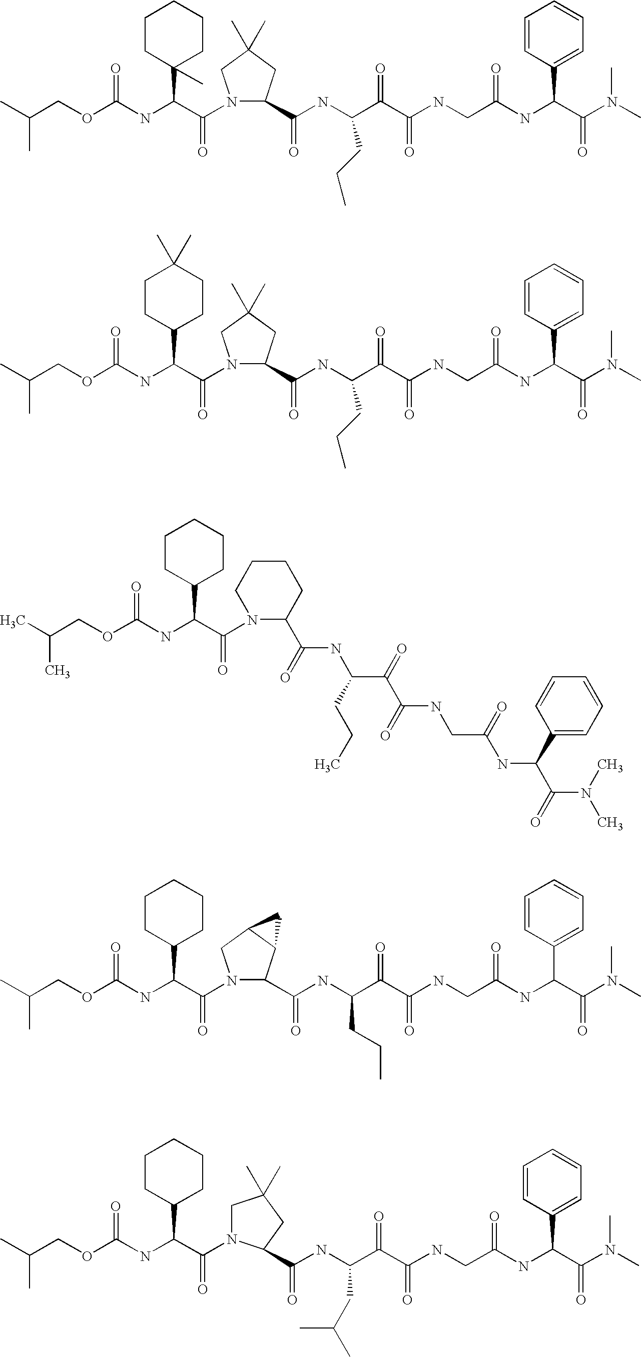 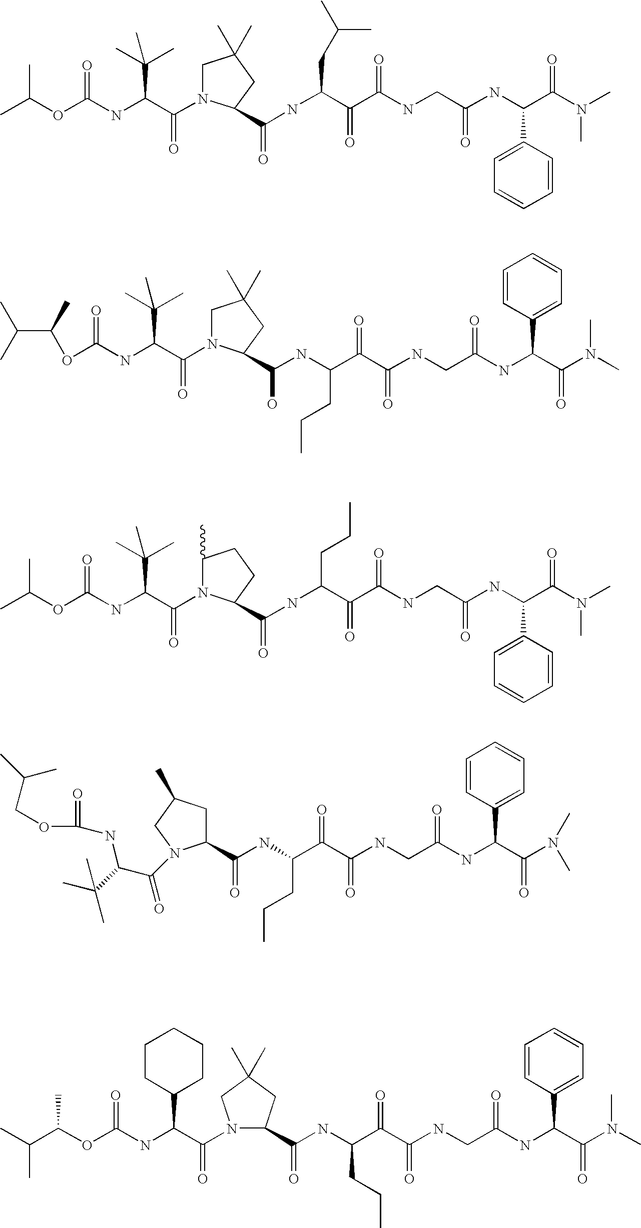 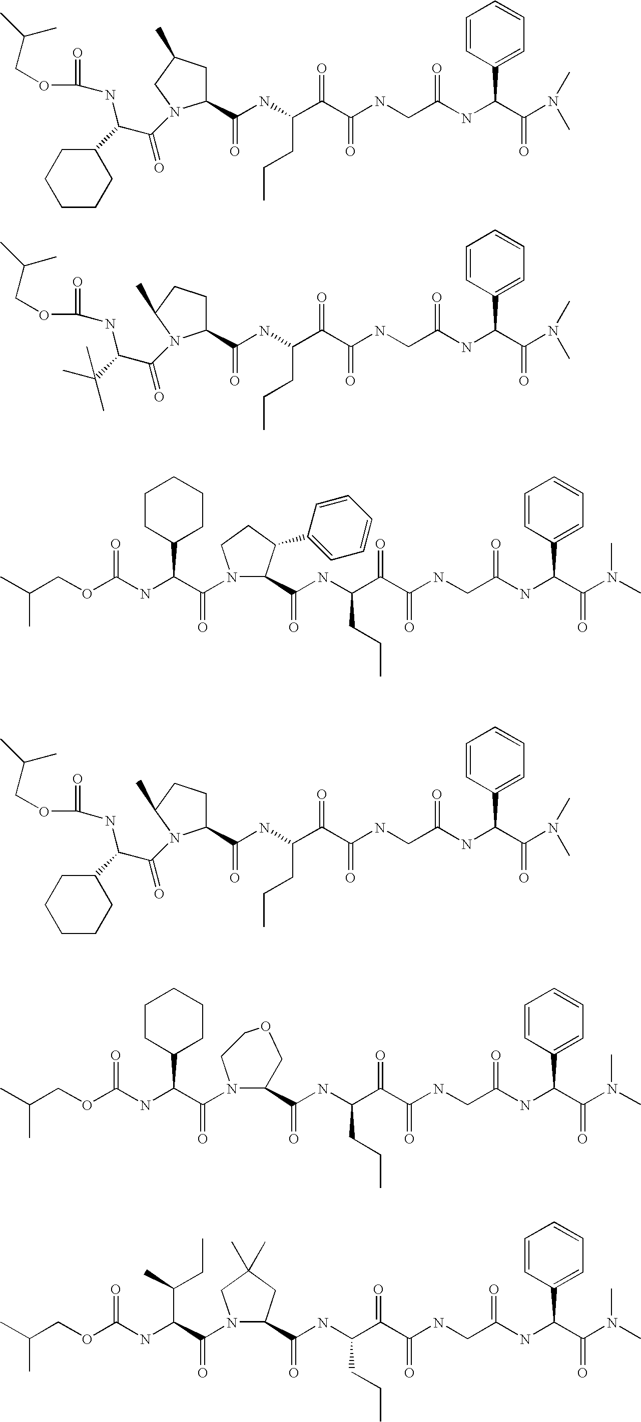 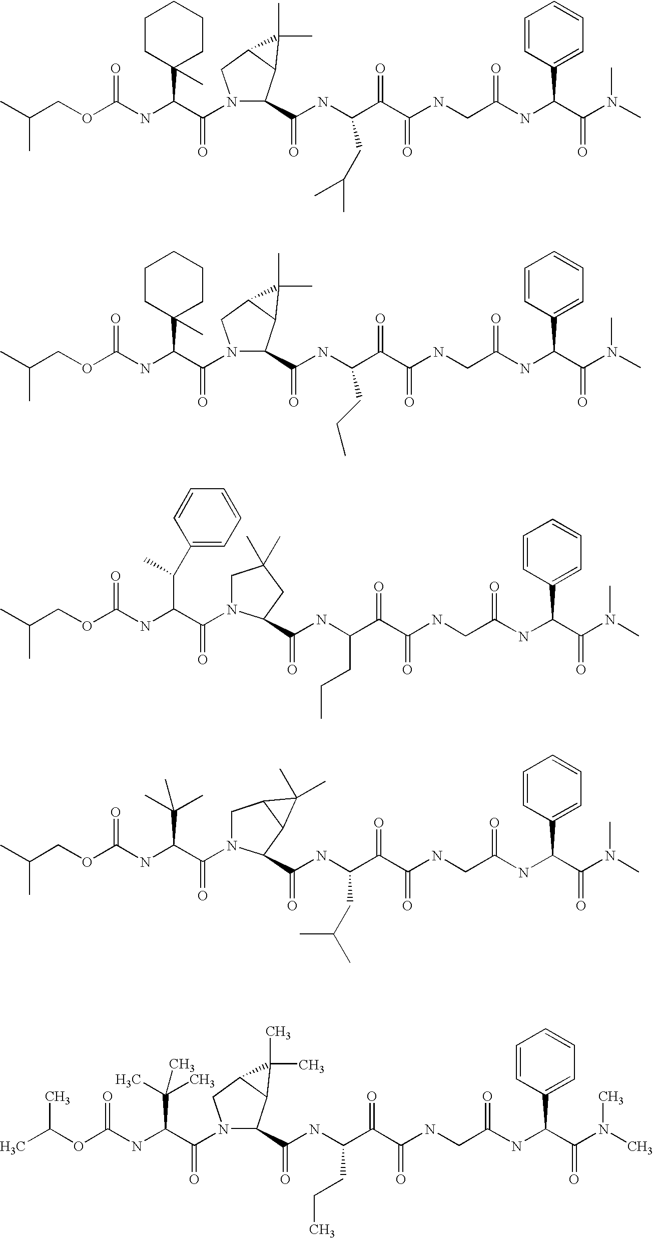 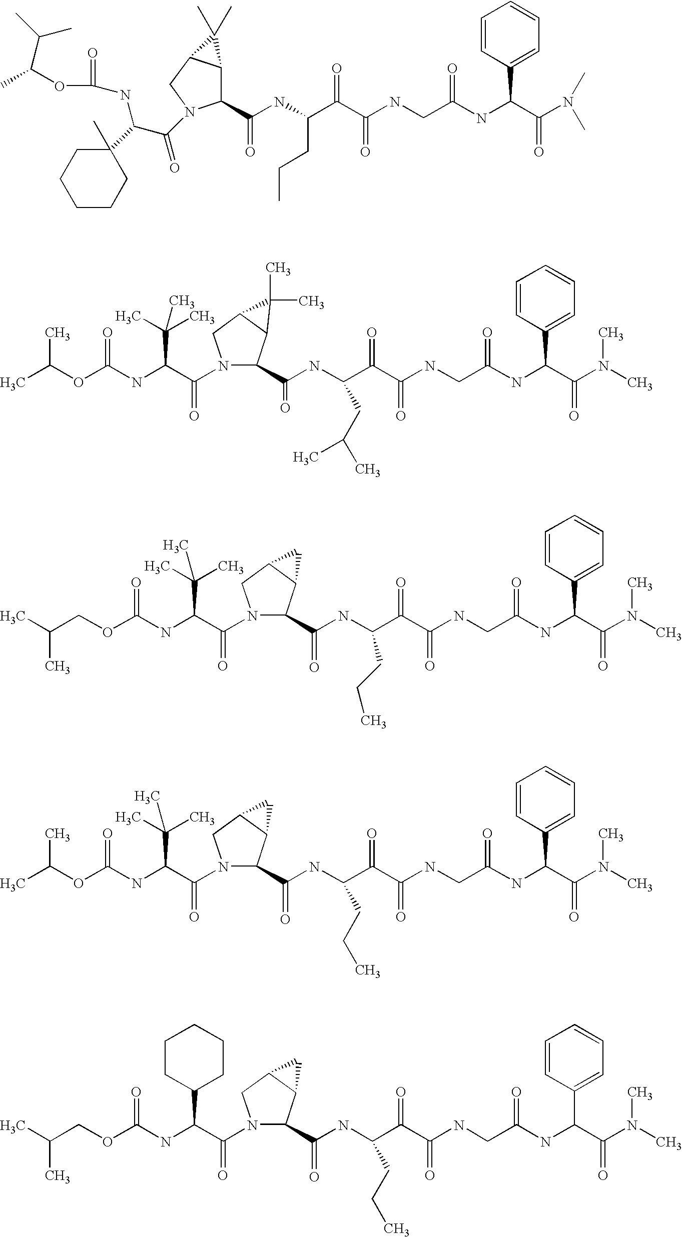 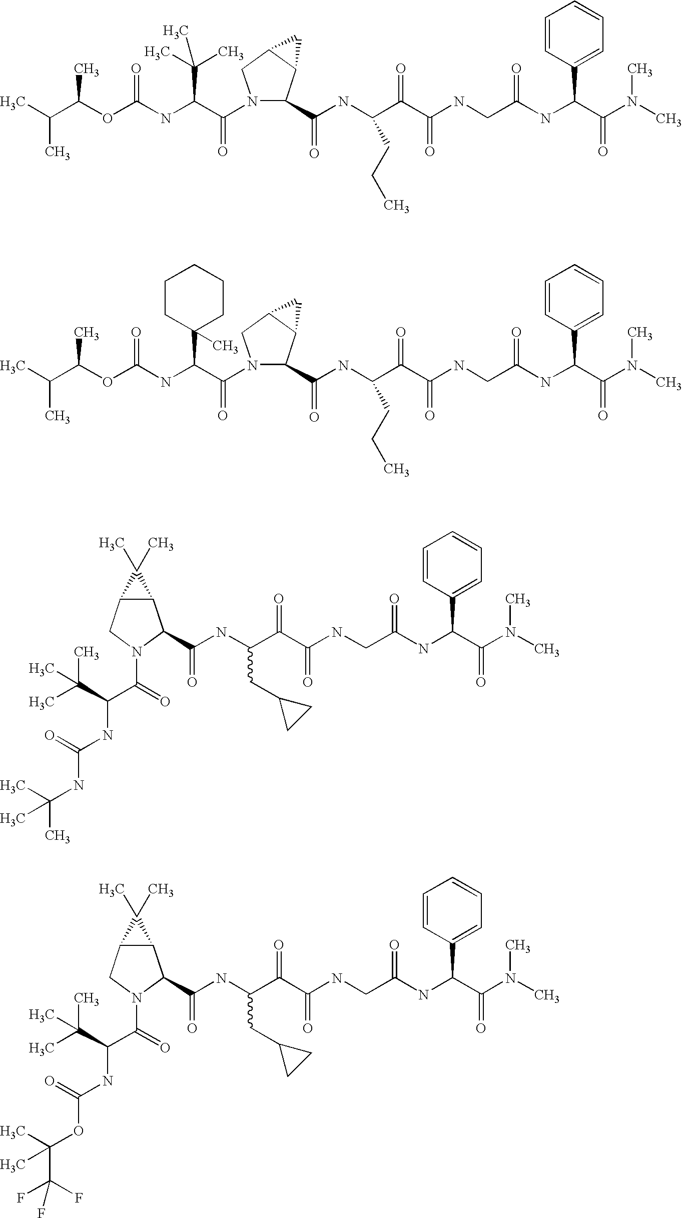 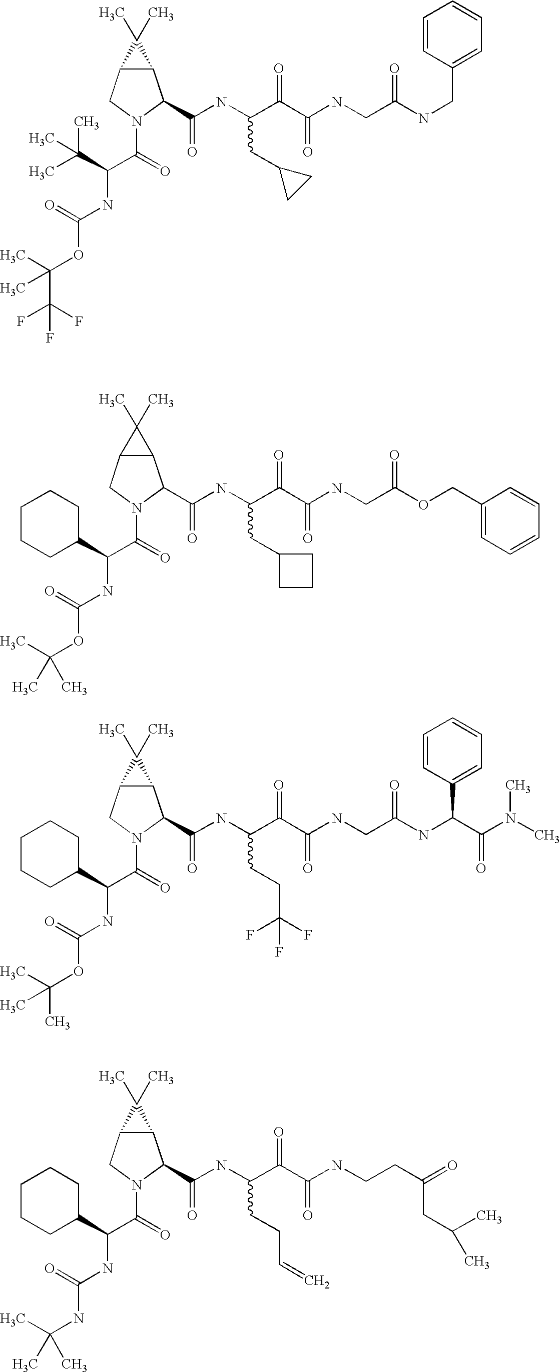 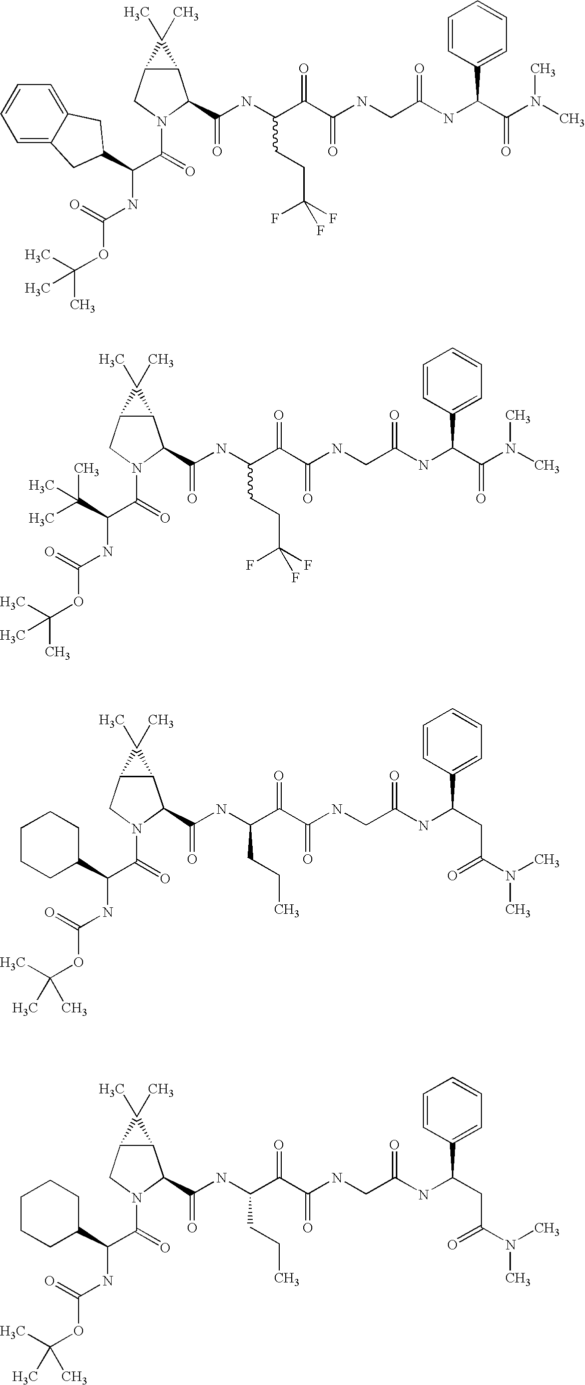 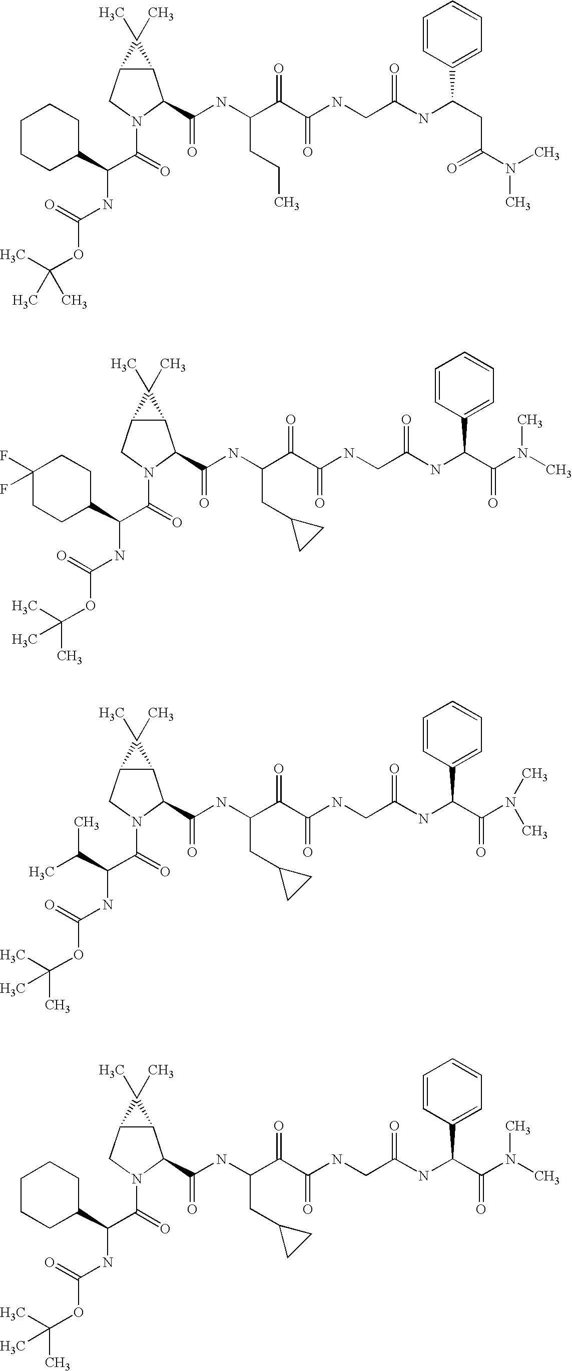 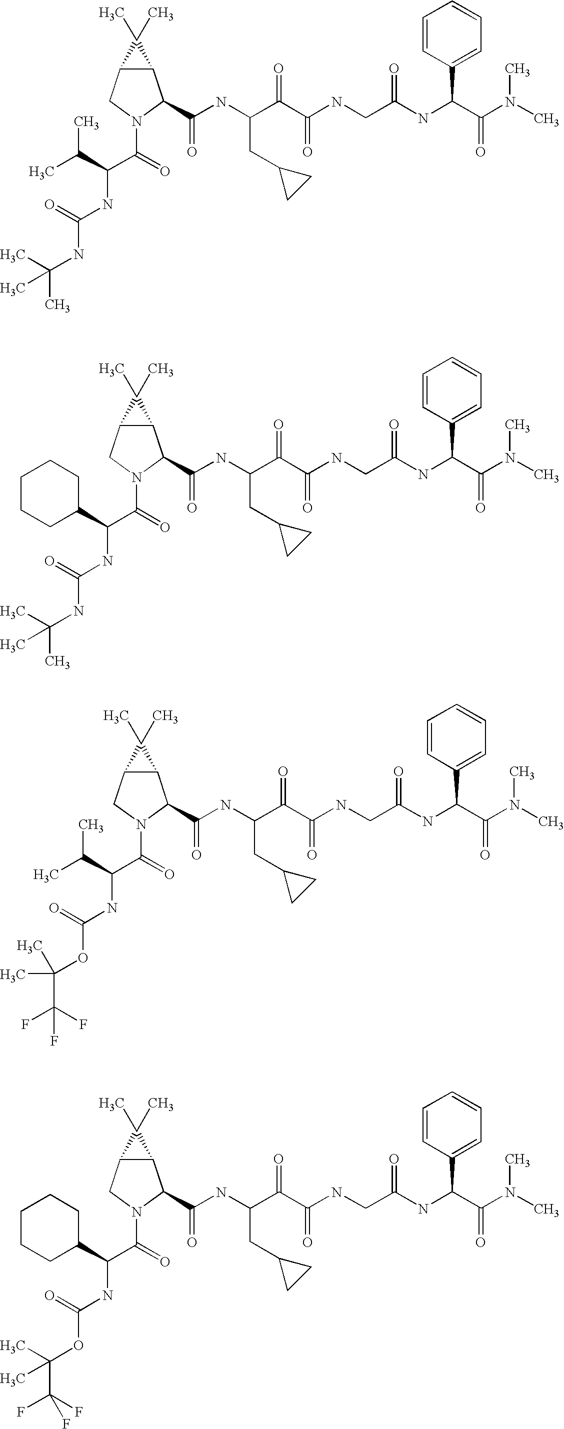 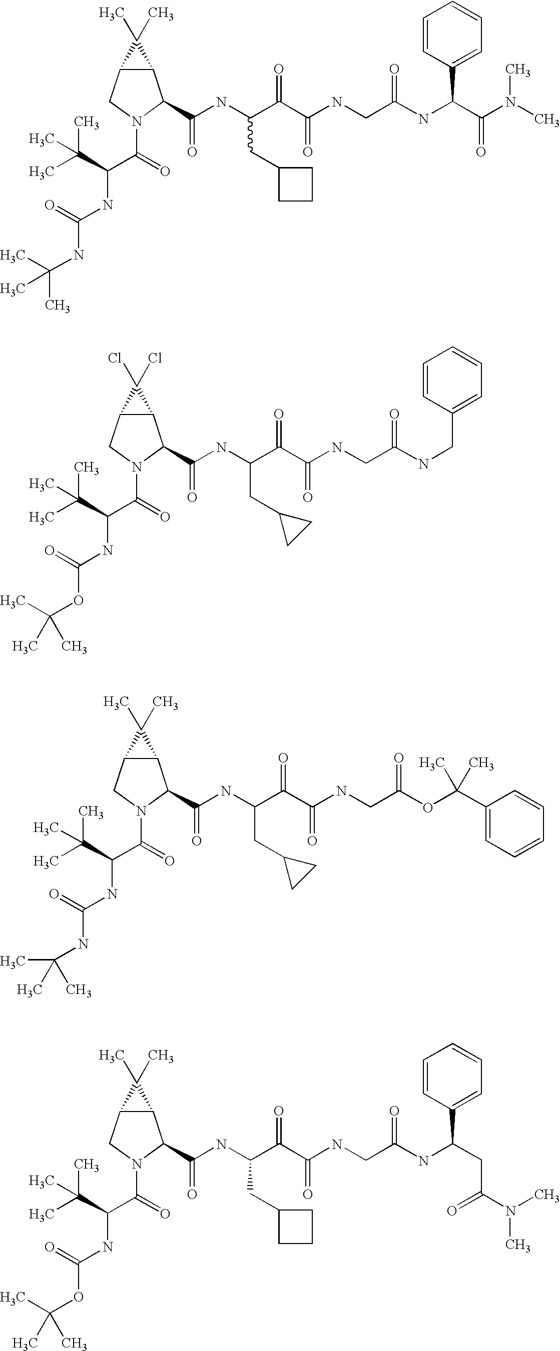 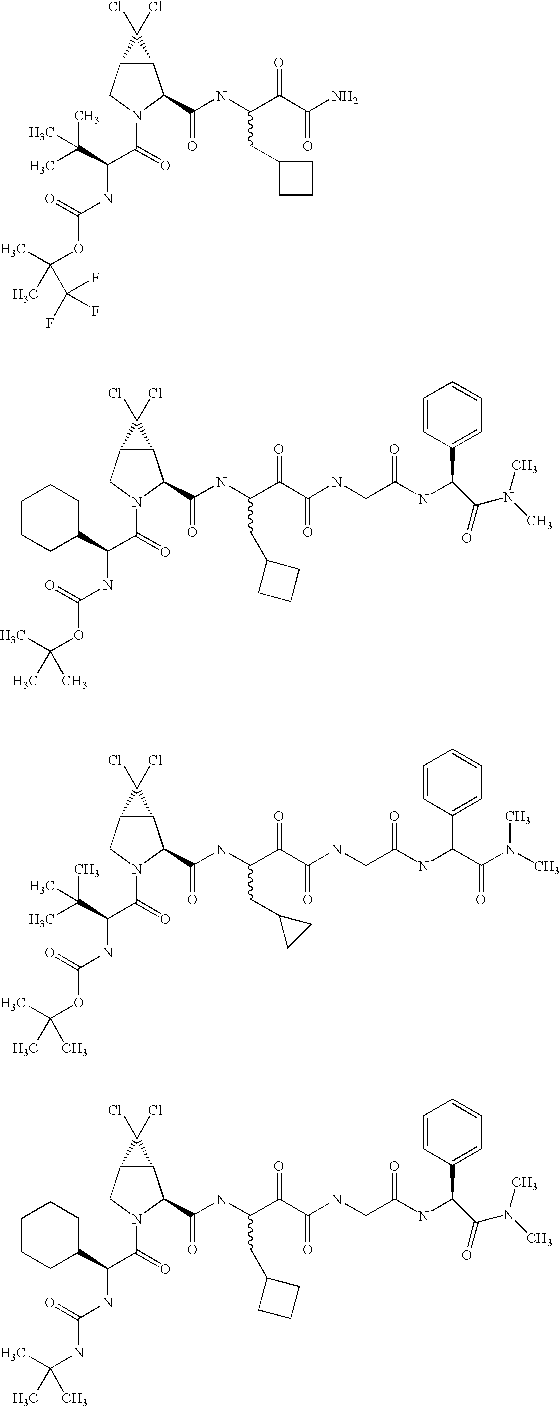 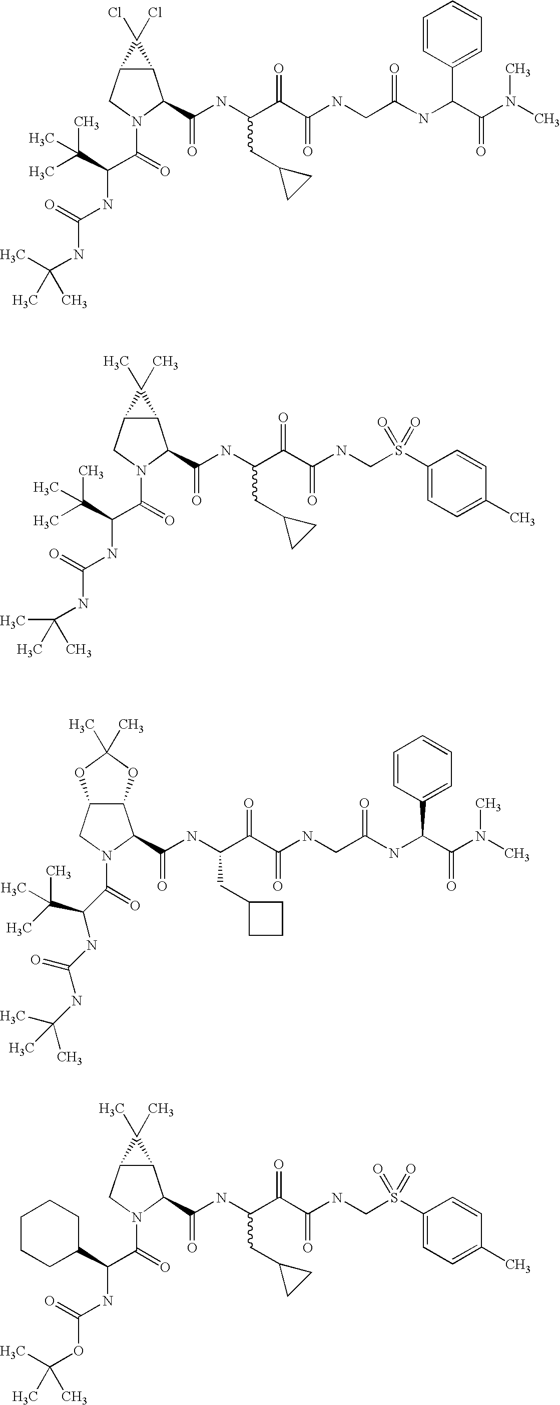 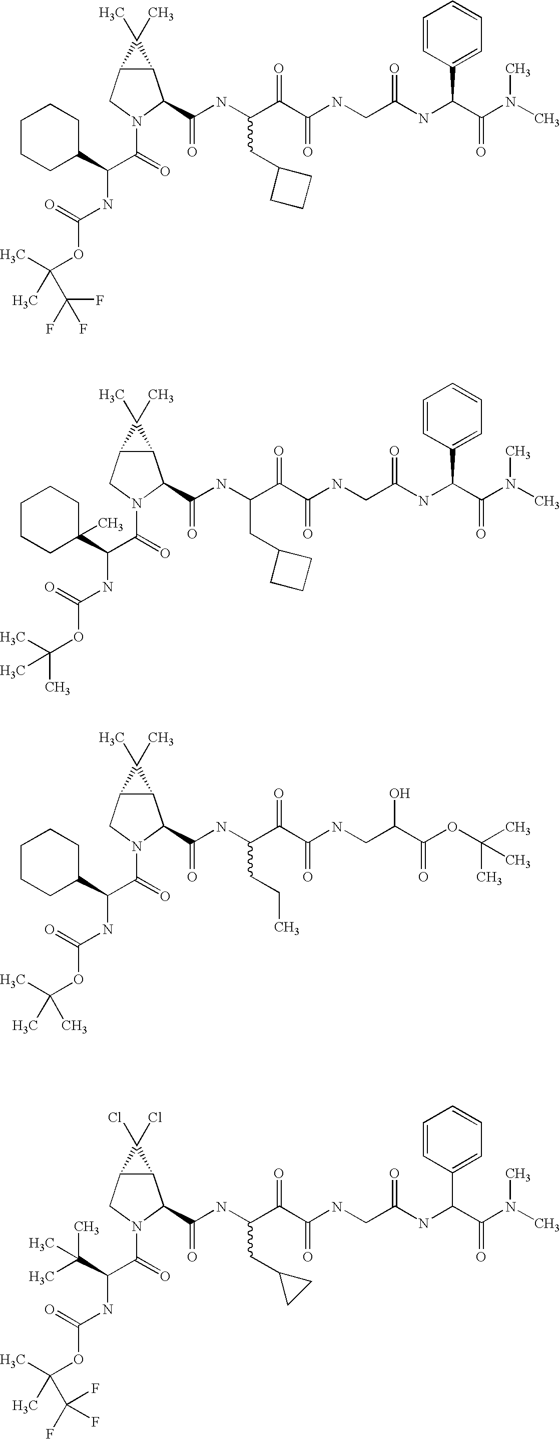 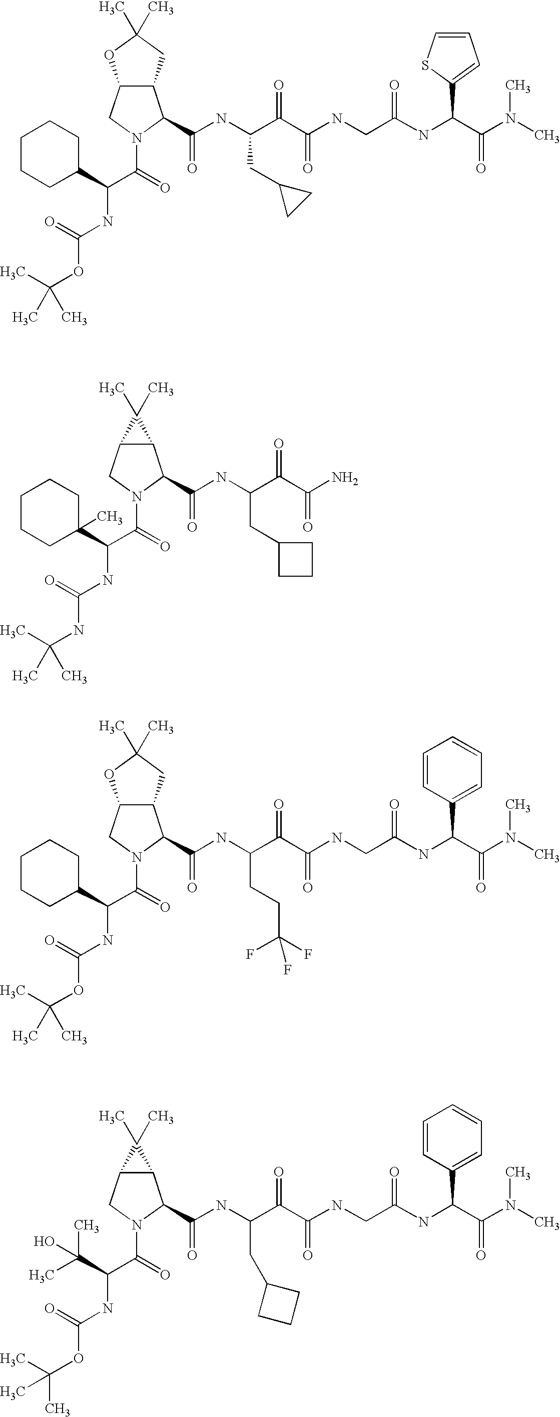 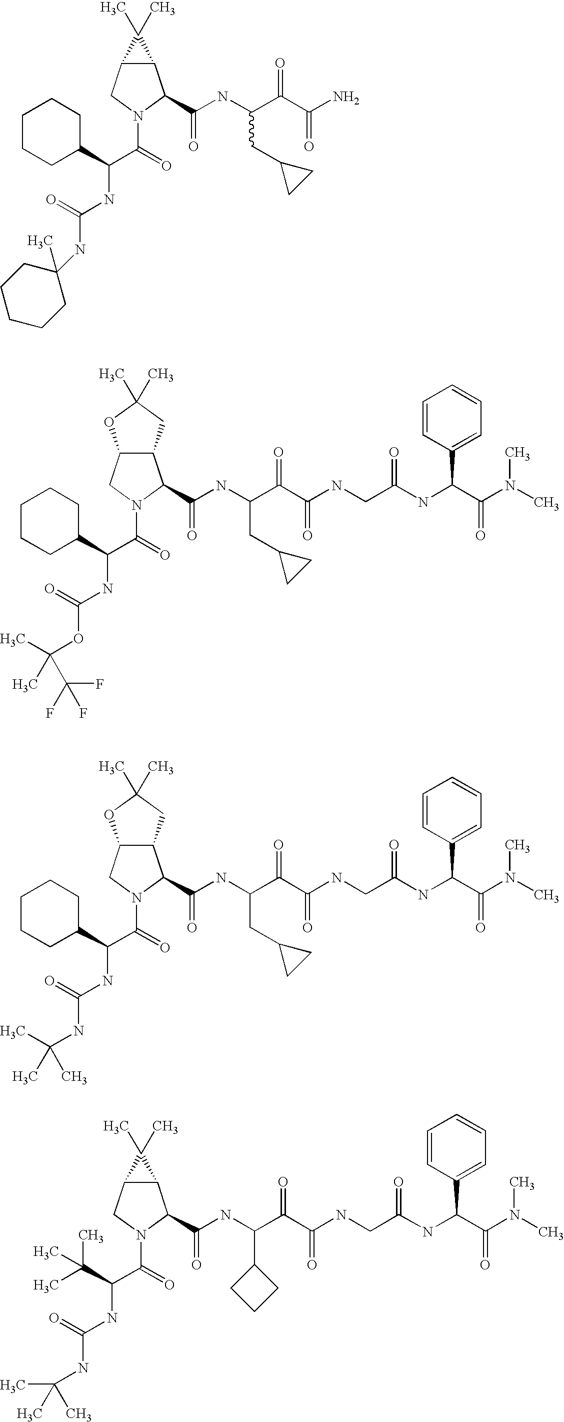 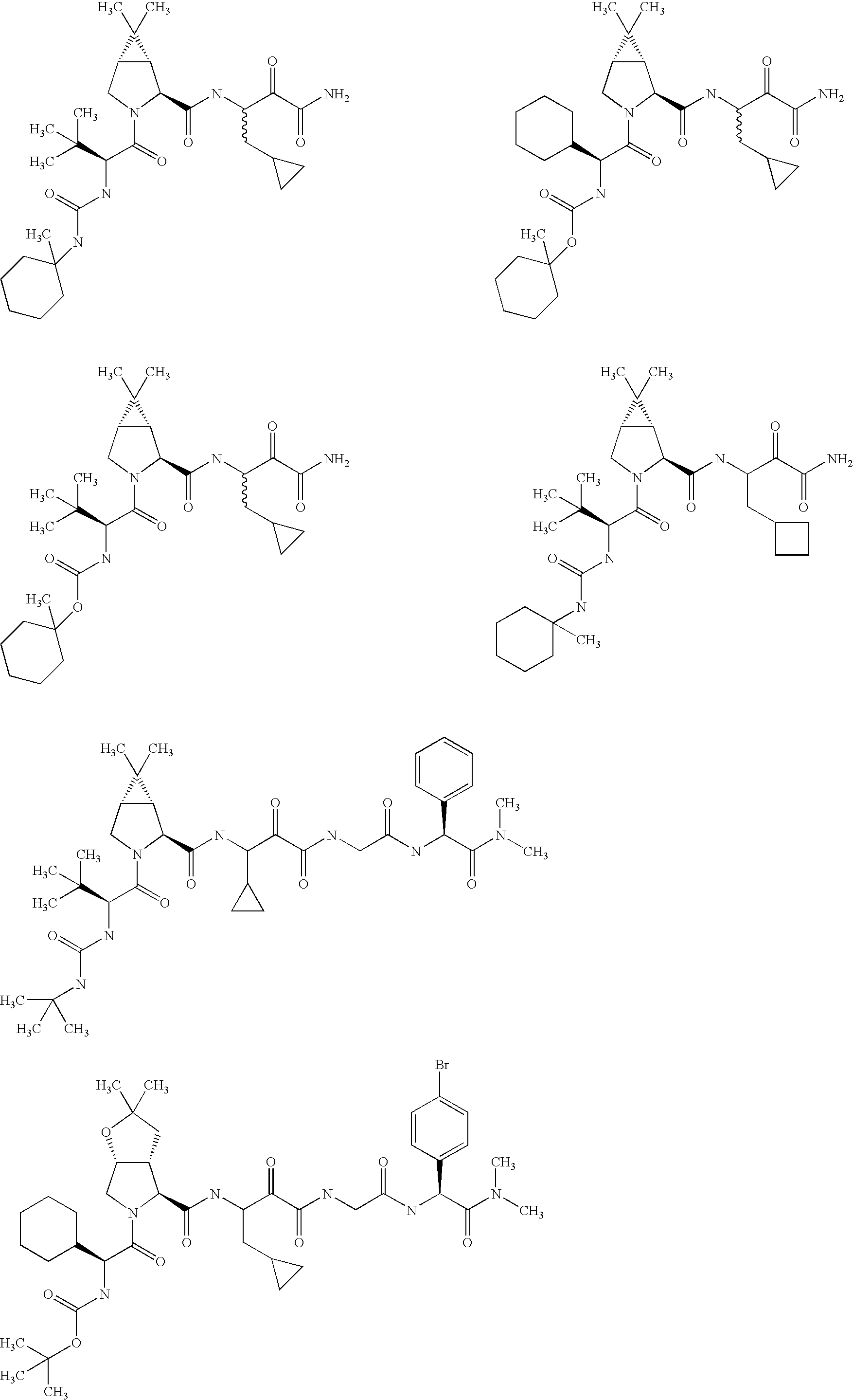 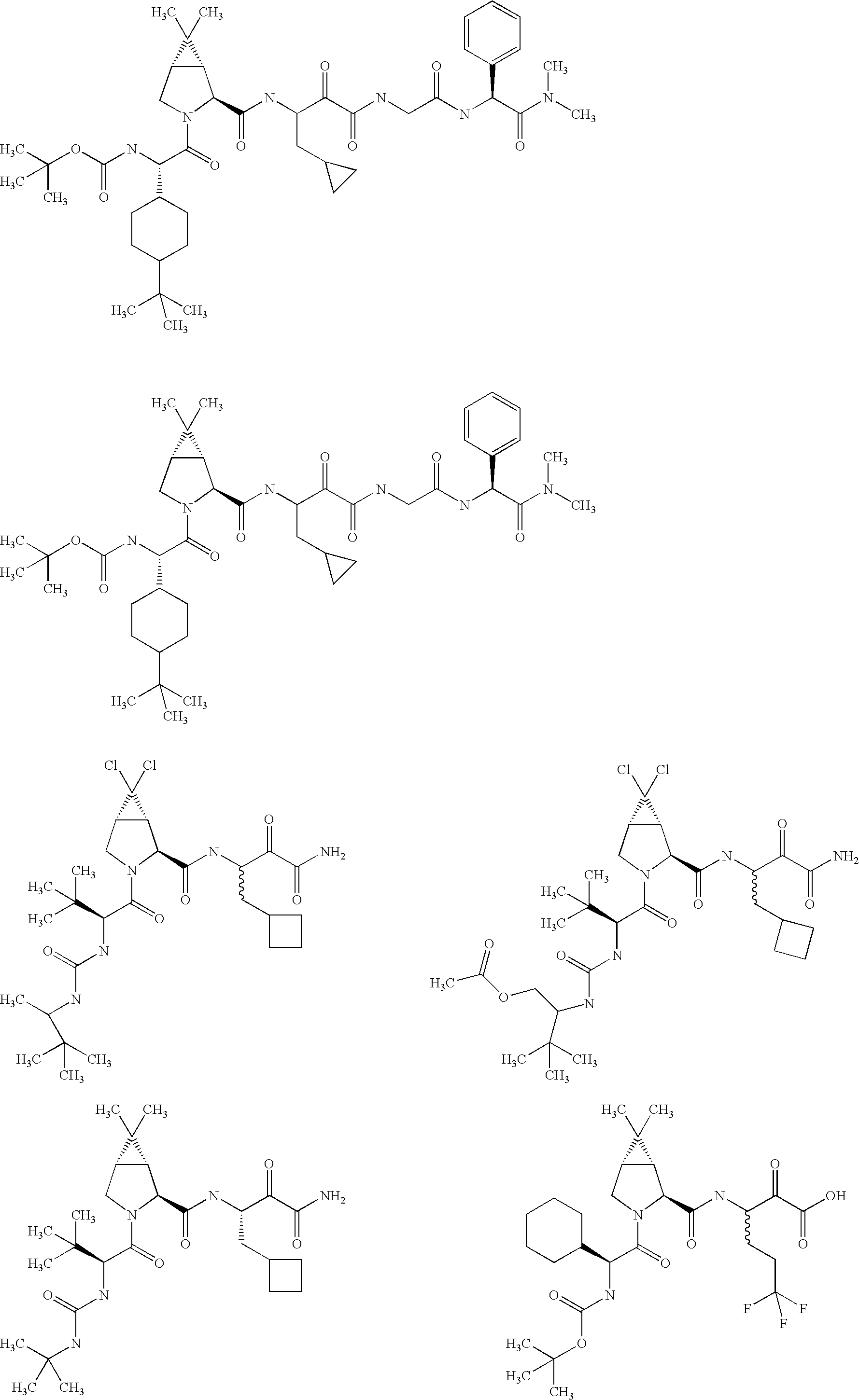 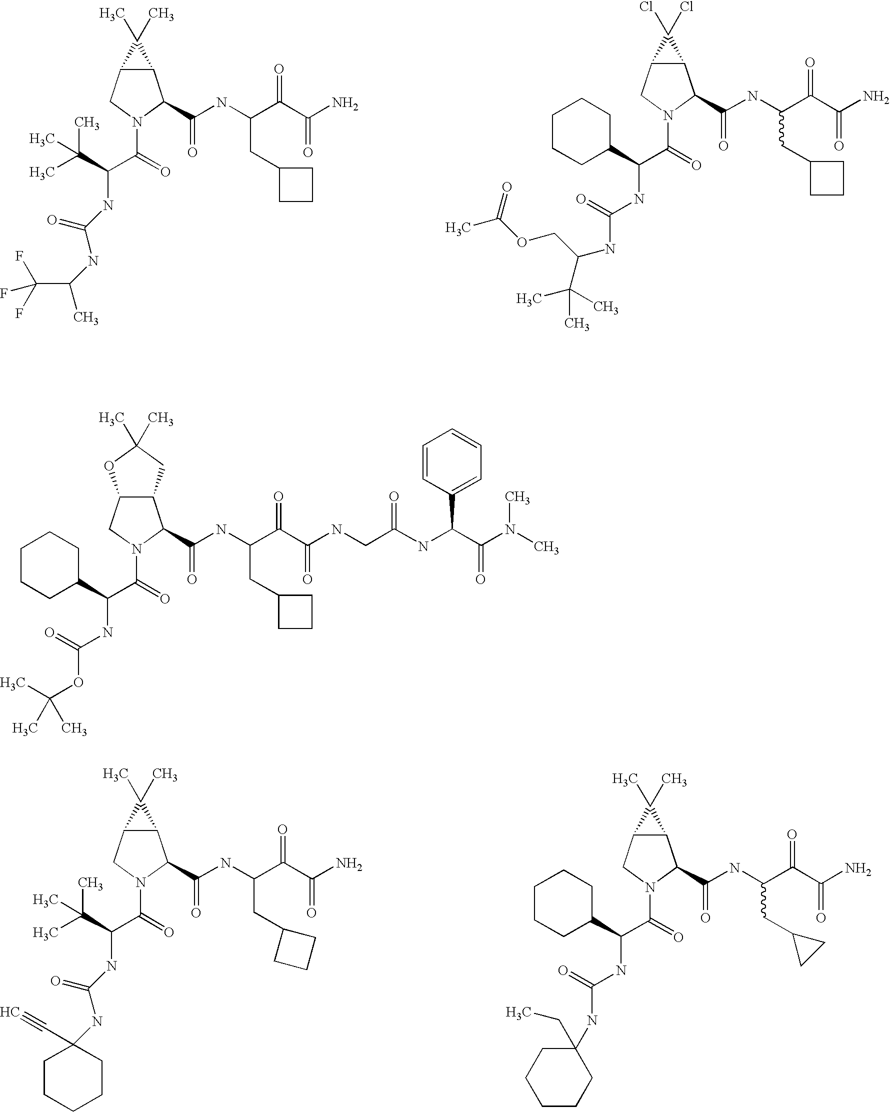 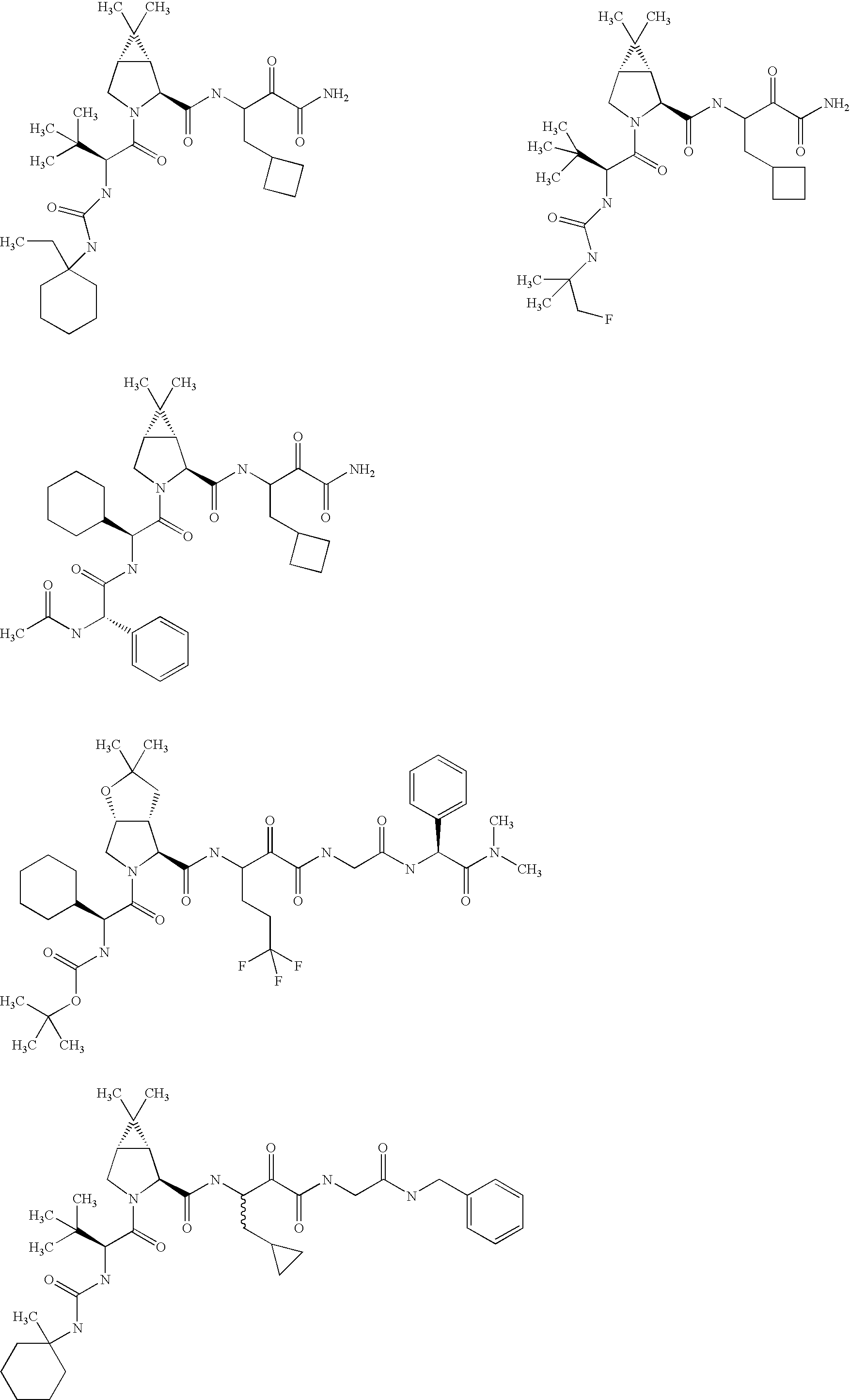 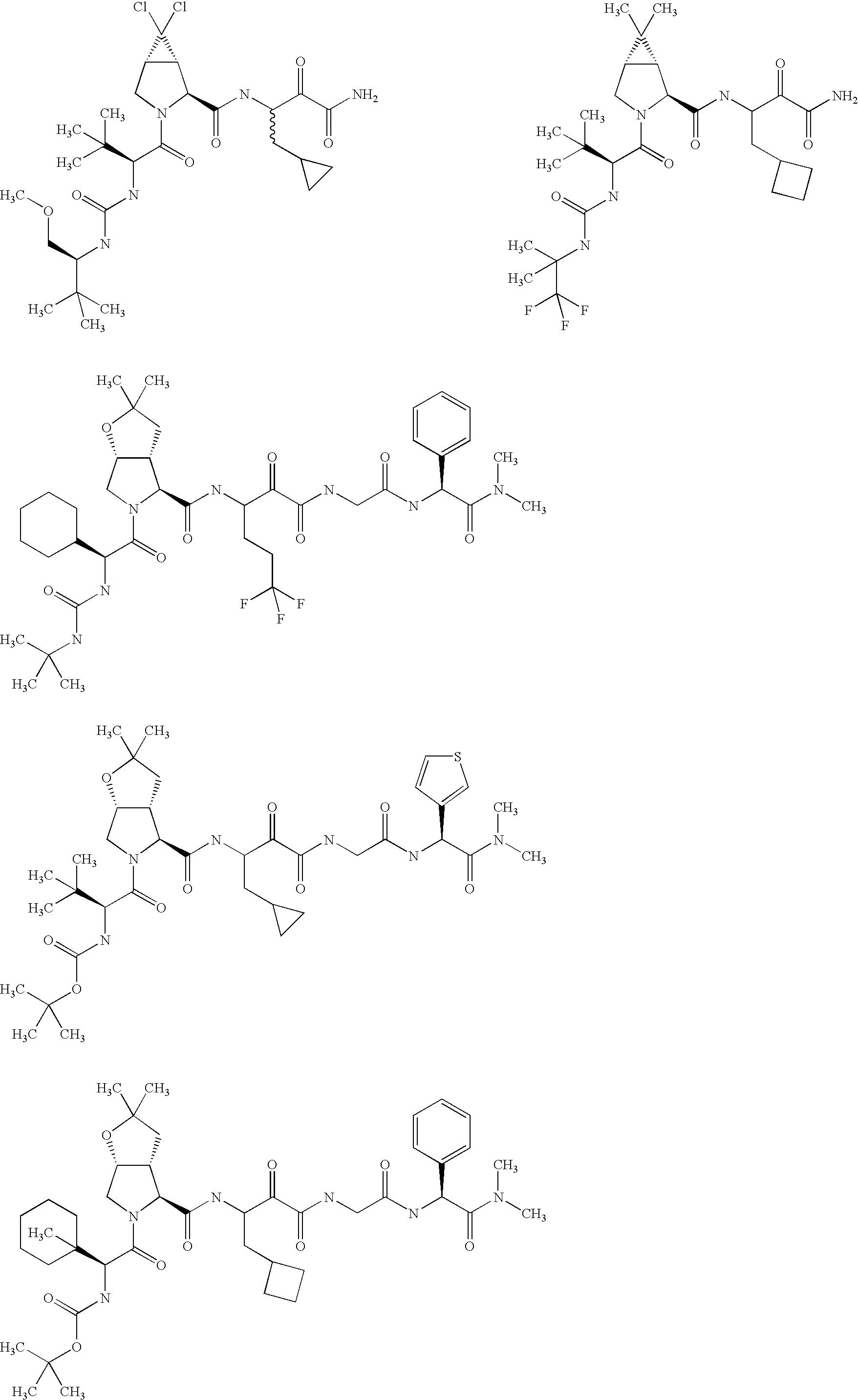 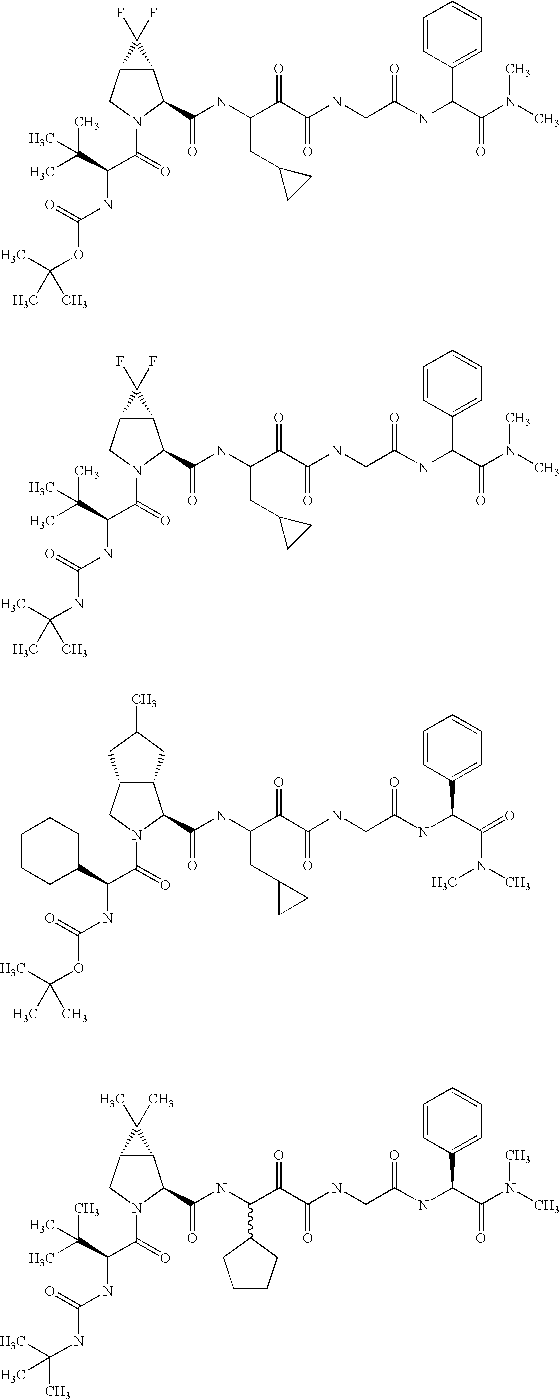 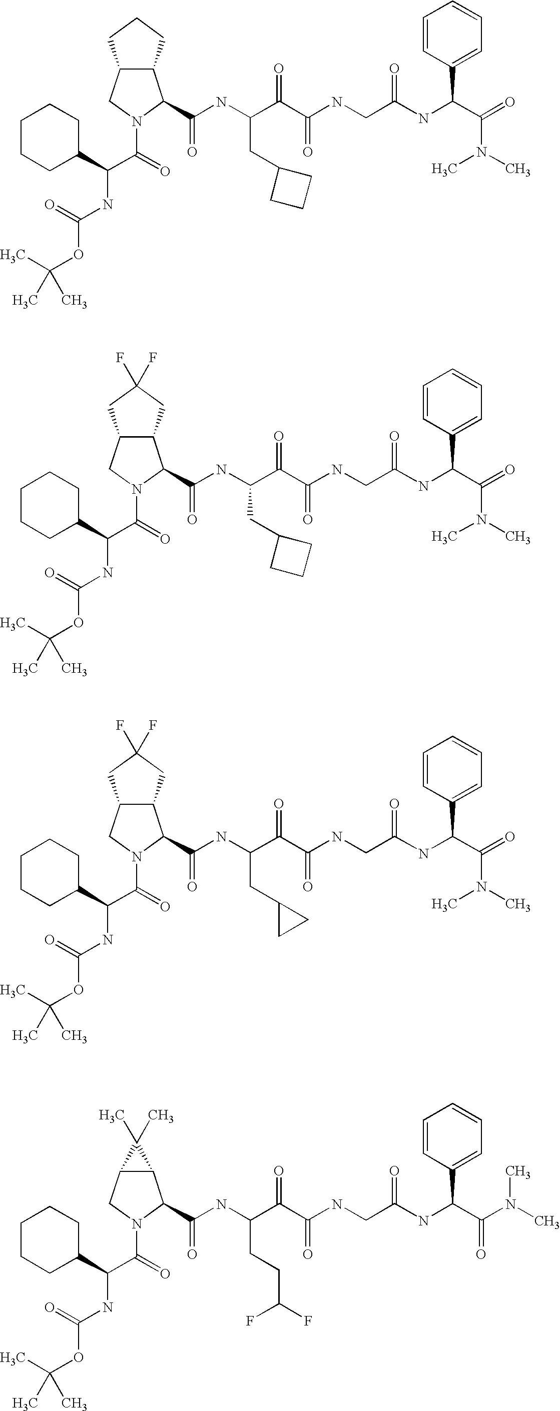 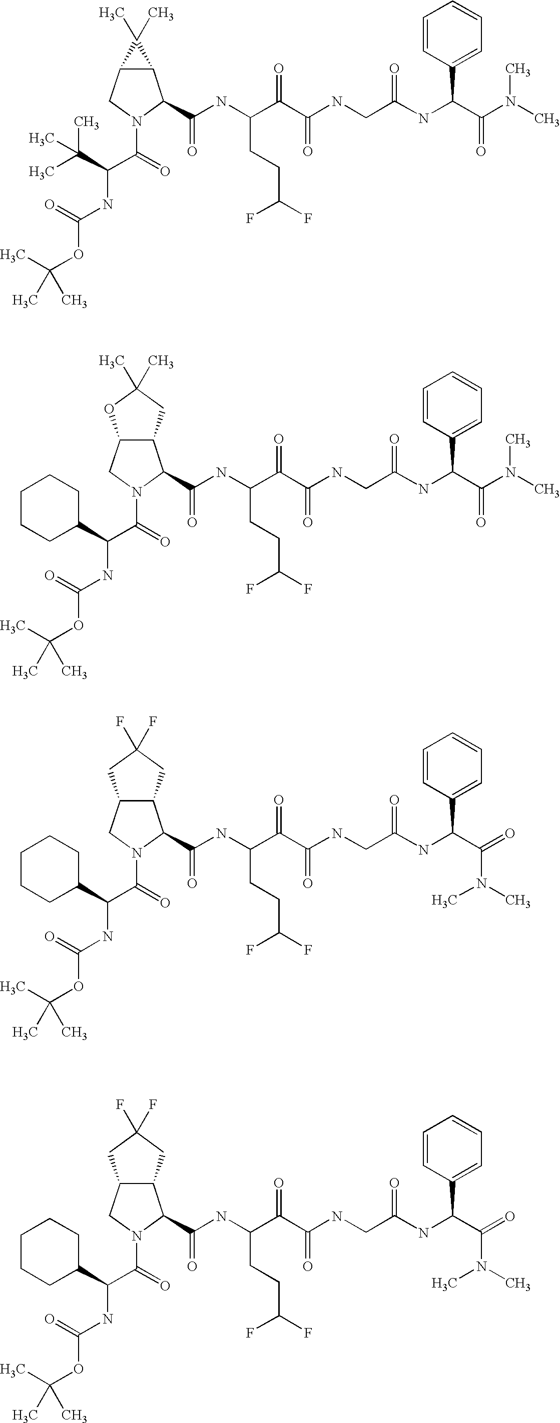 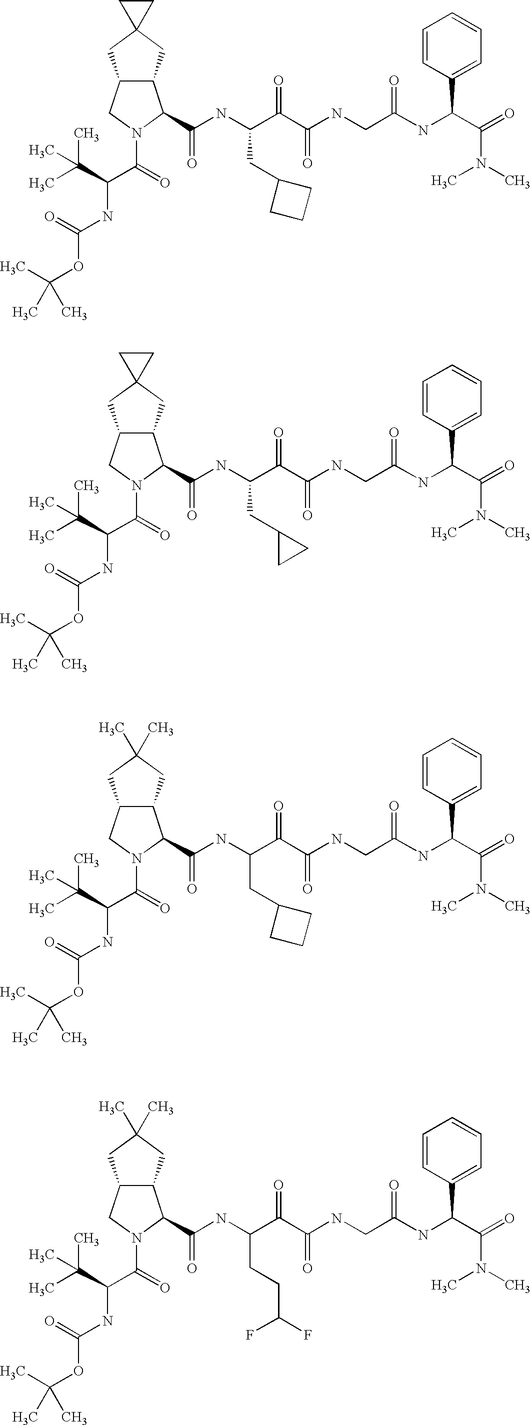 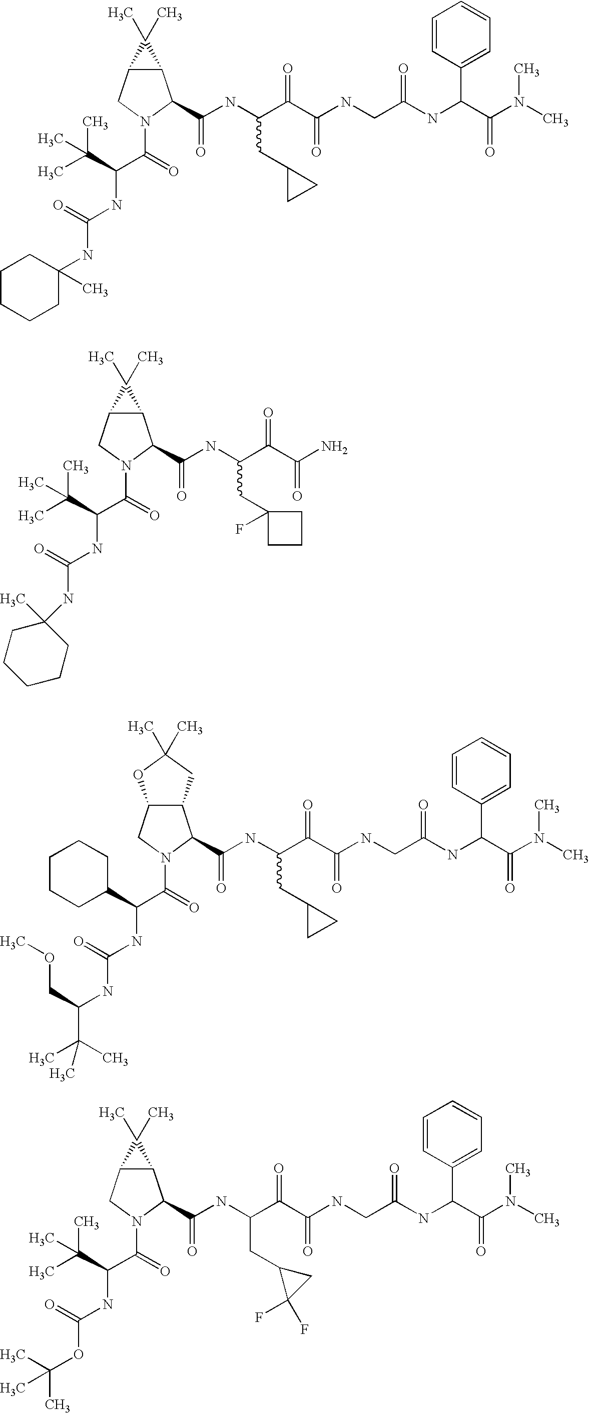 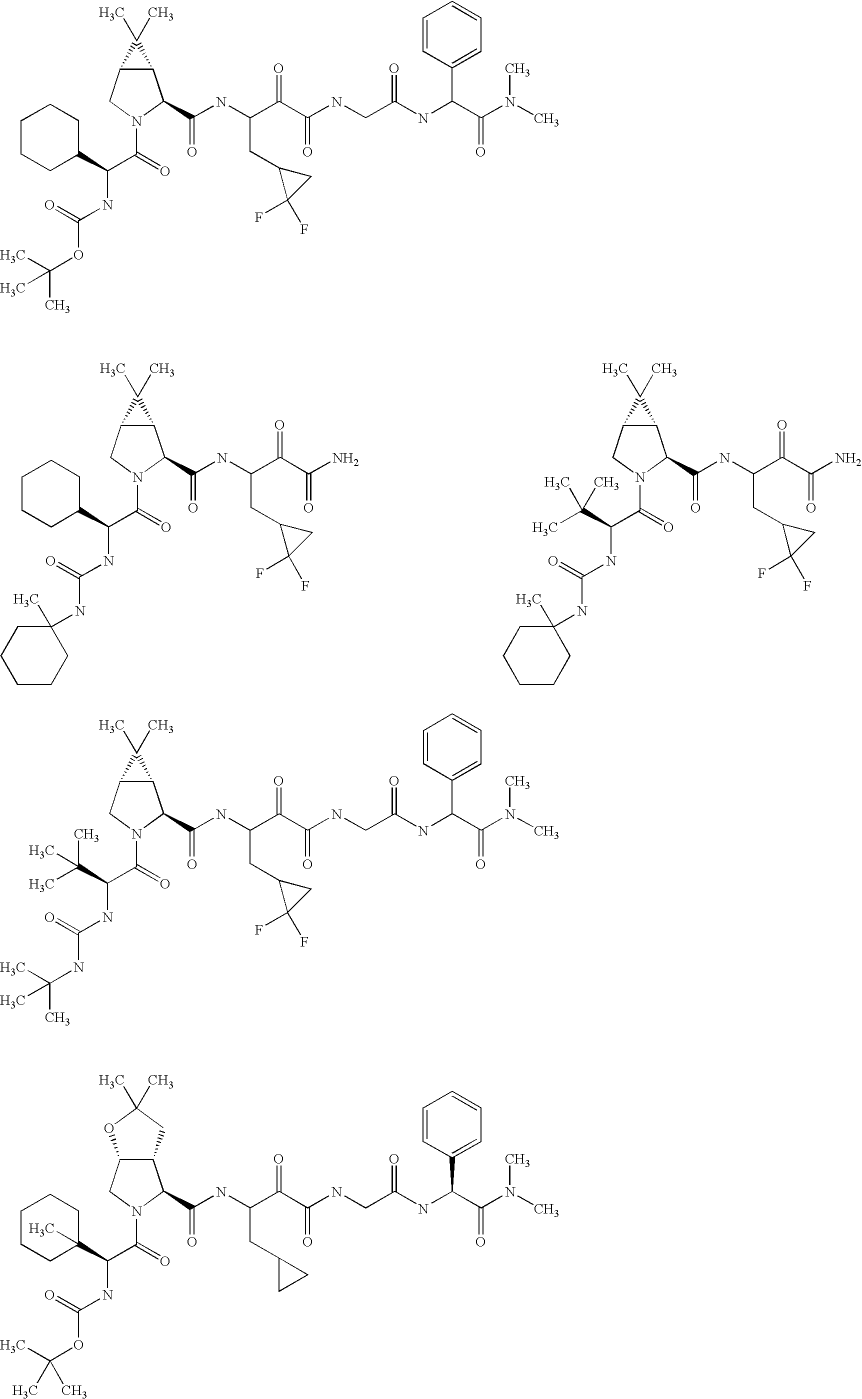 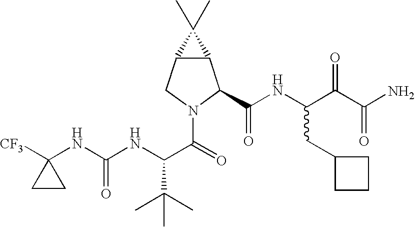
or a pharmaceutically acceptable salt, solvate or ester thereof.

The compounds of formula II are disclosed in WO02/08256 published Jan. 31, 2002, and in U.S. Pat. No. 6,800,434, granted on Oct. 5, 2004, both of which are incorporated herein by reference.

Non-limiting examples of certain compounds disclosed in U.S. Pat. No. 6,800,434 are: 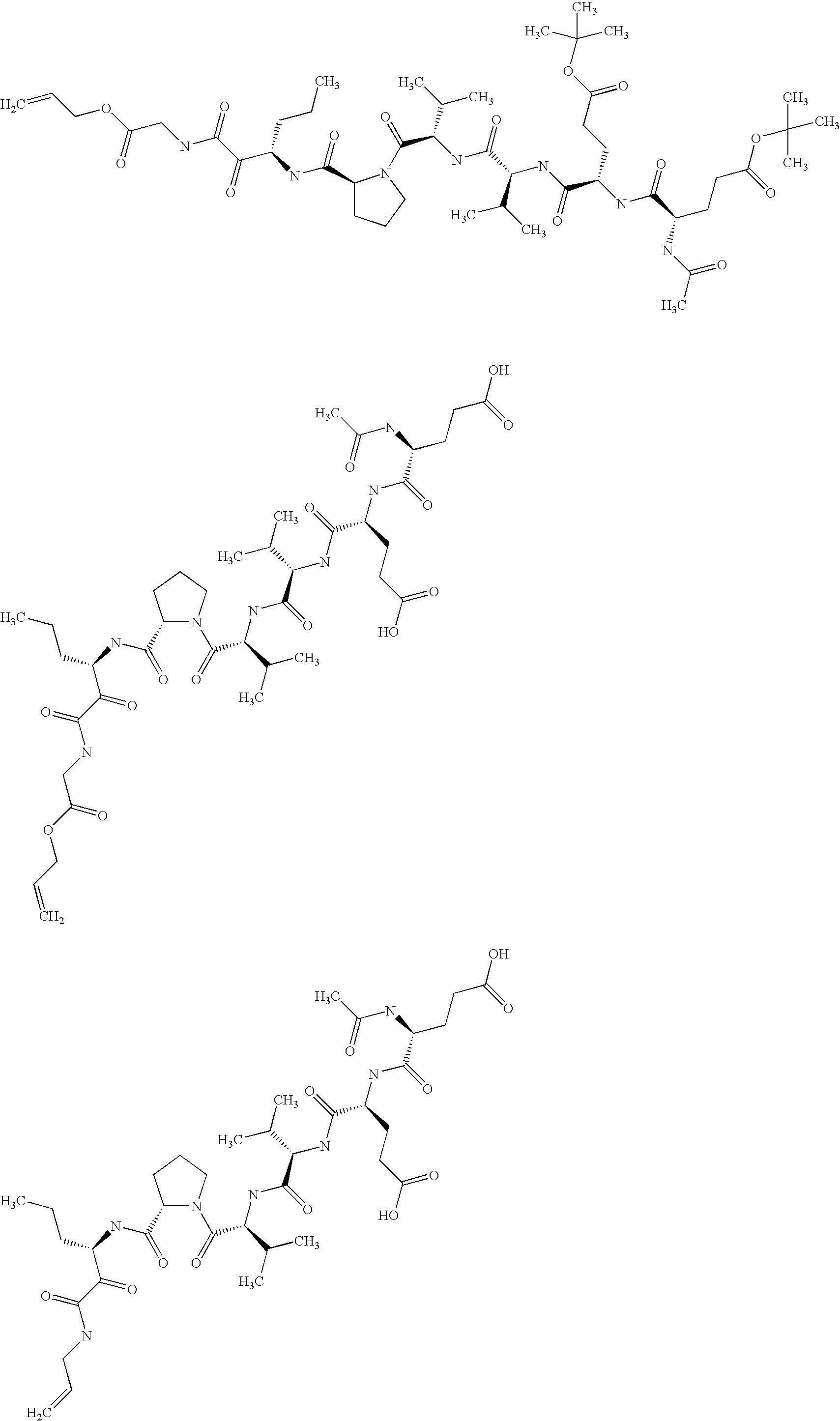 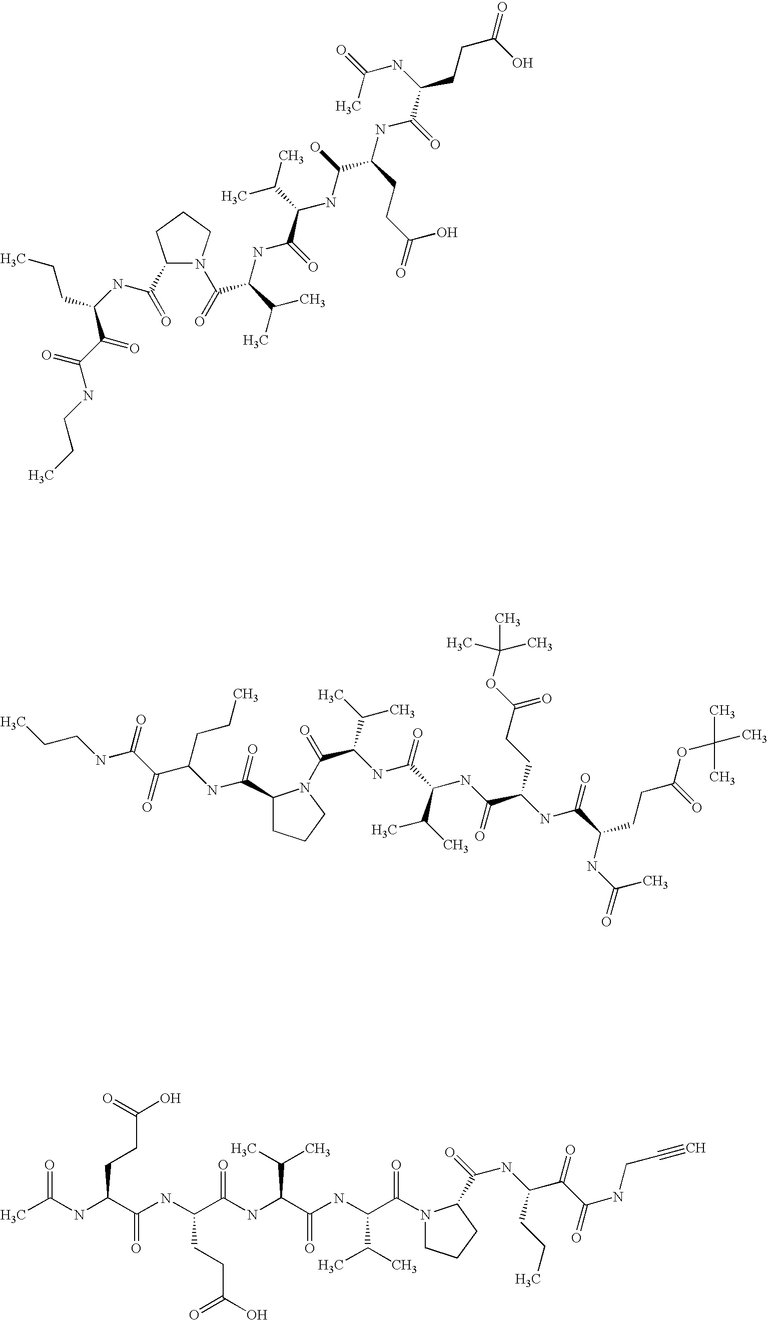 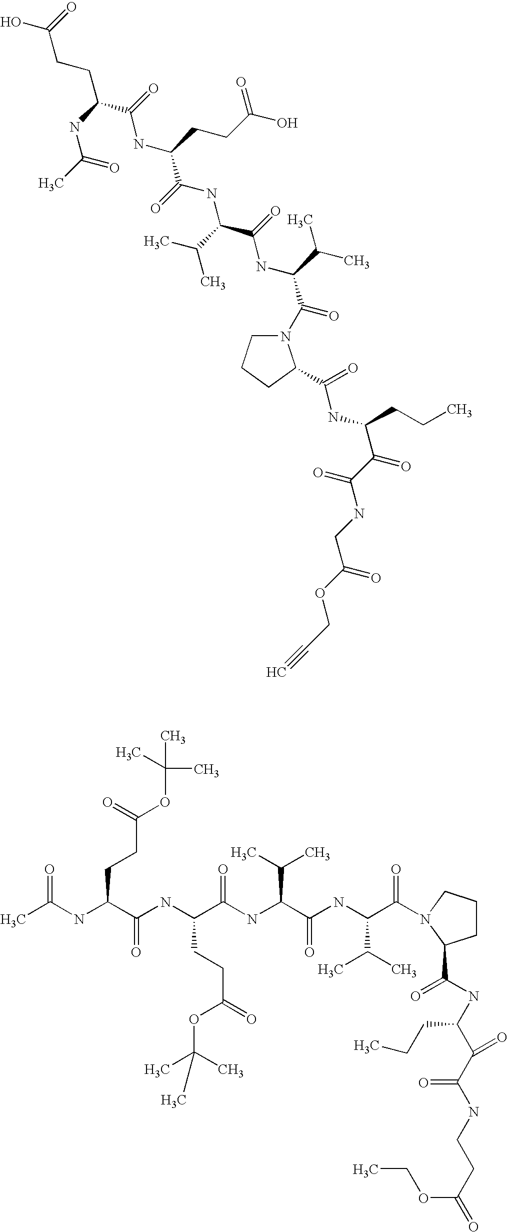 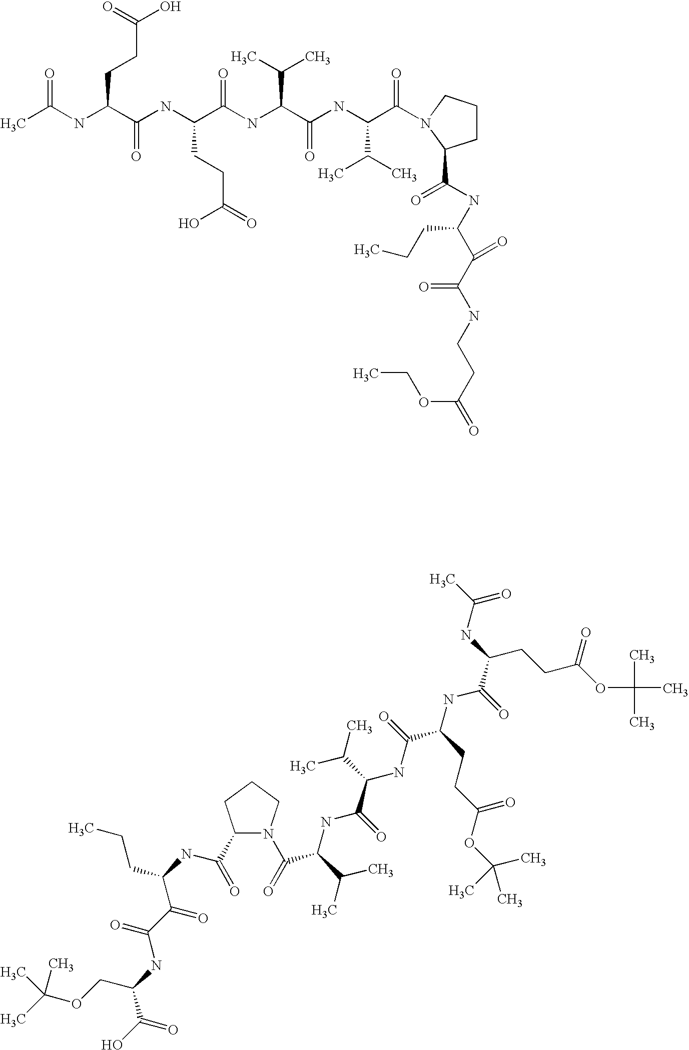 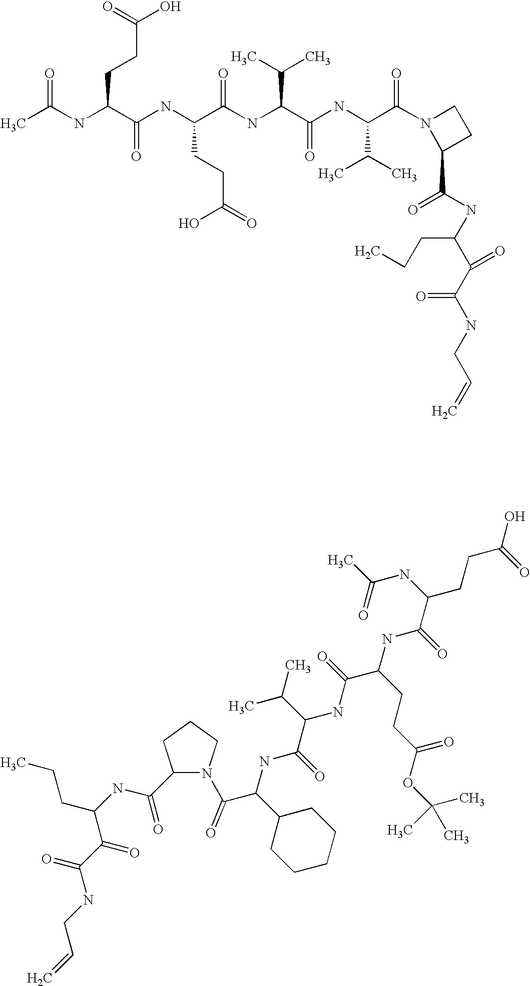 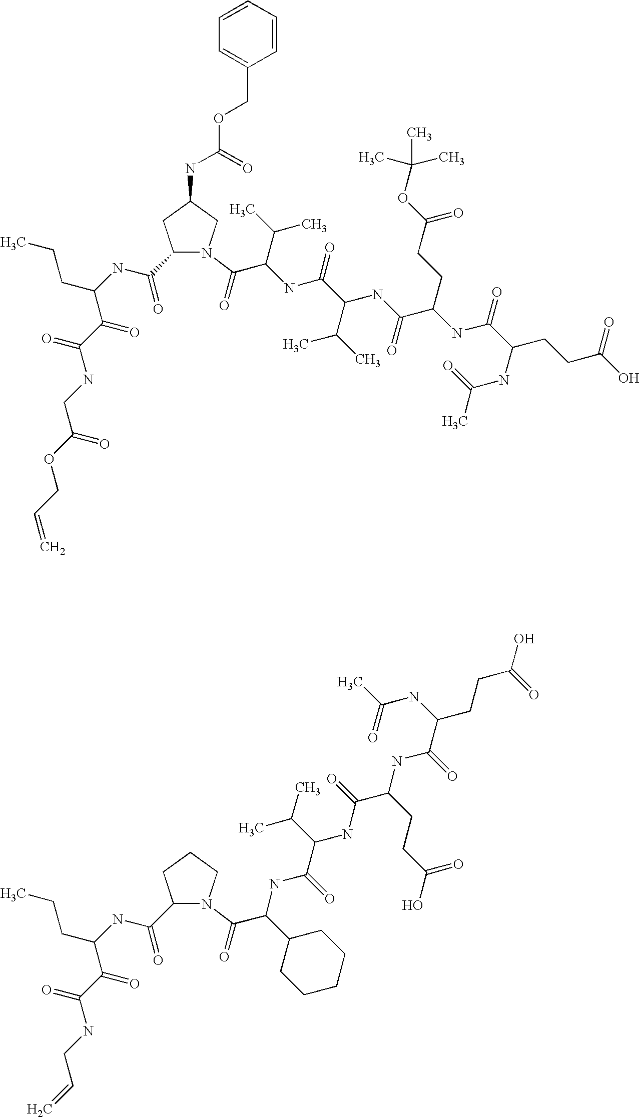 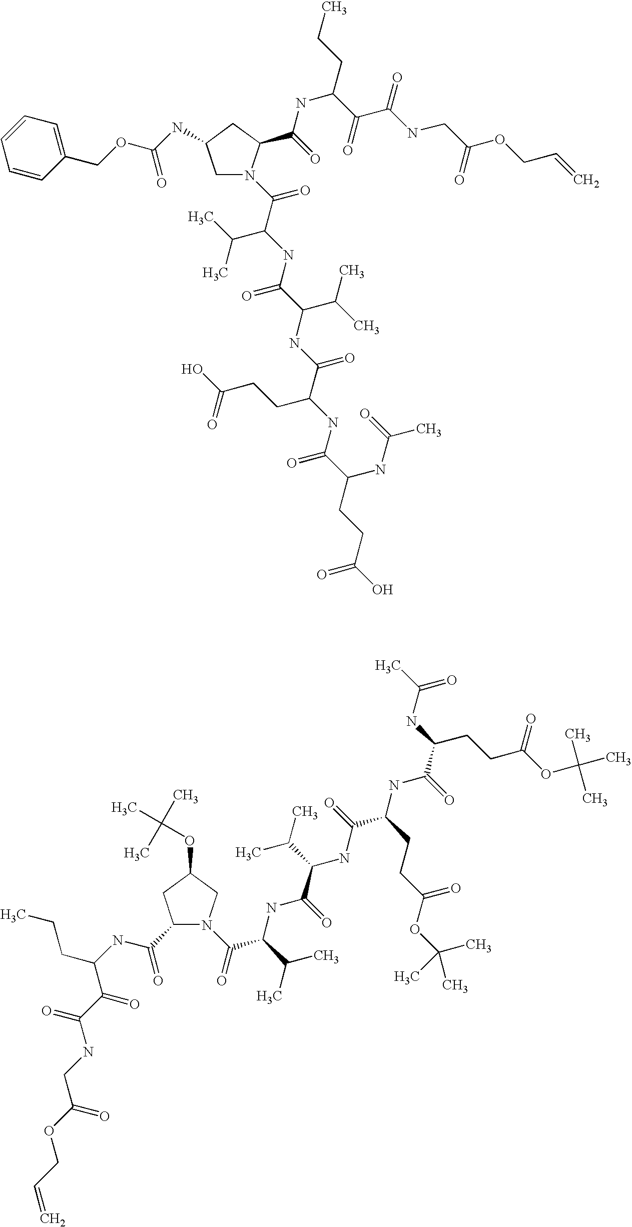 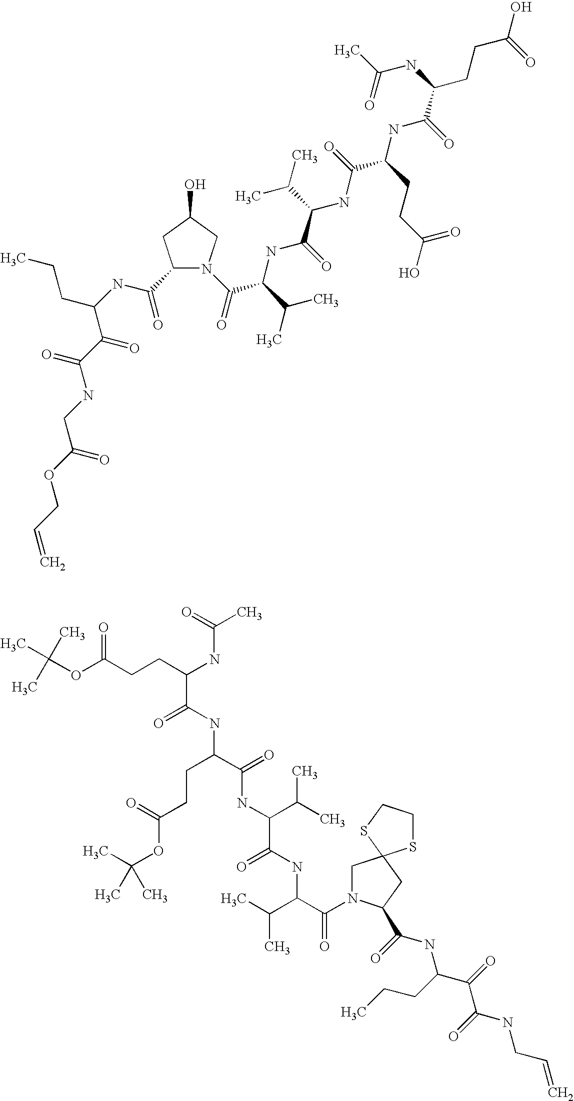 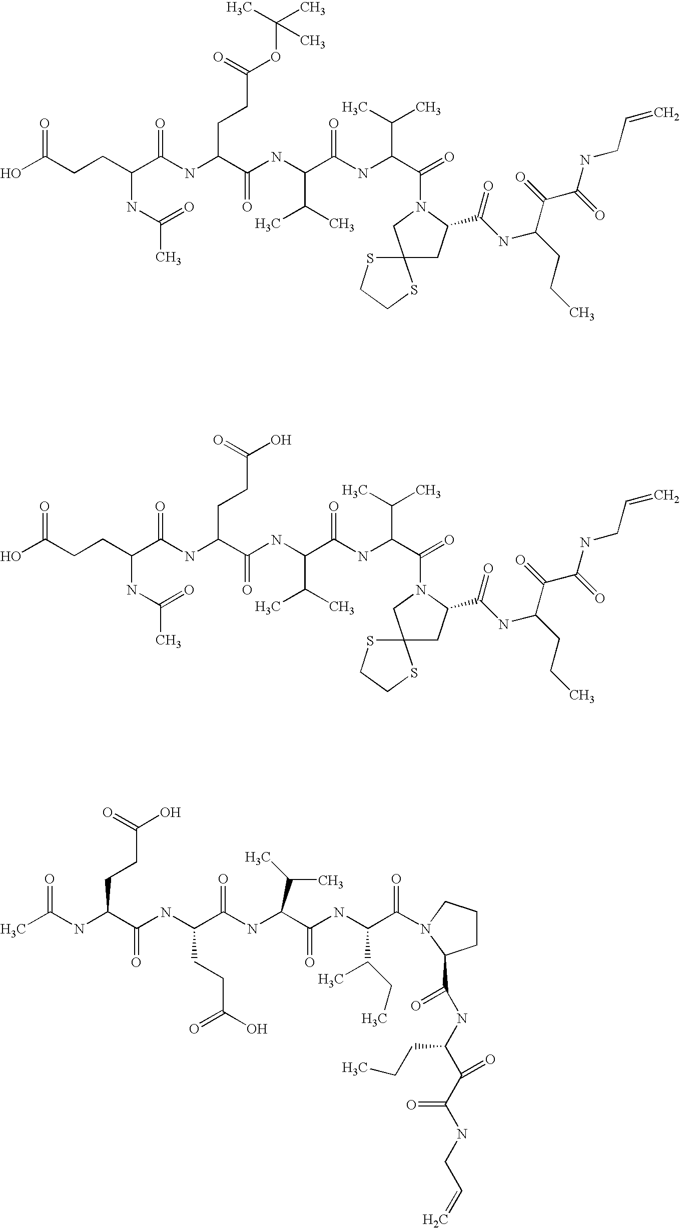 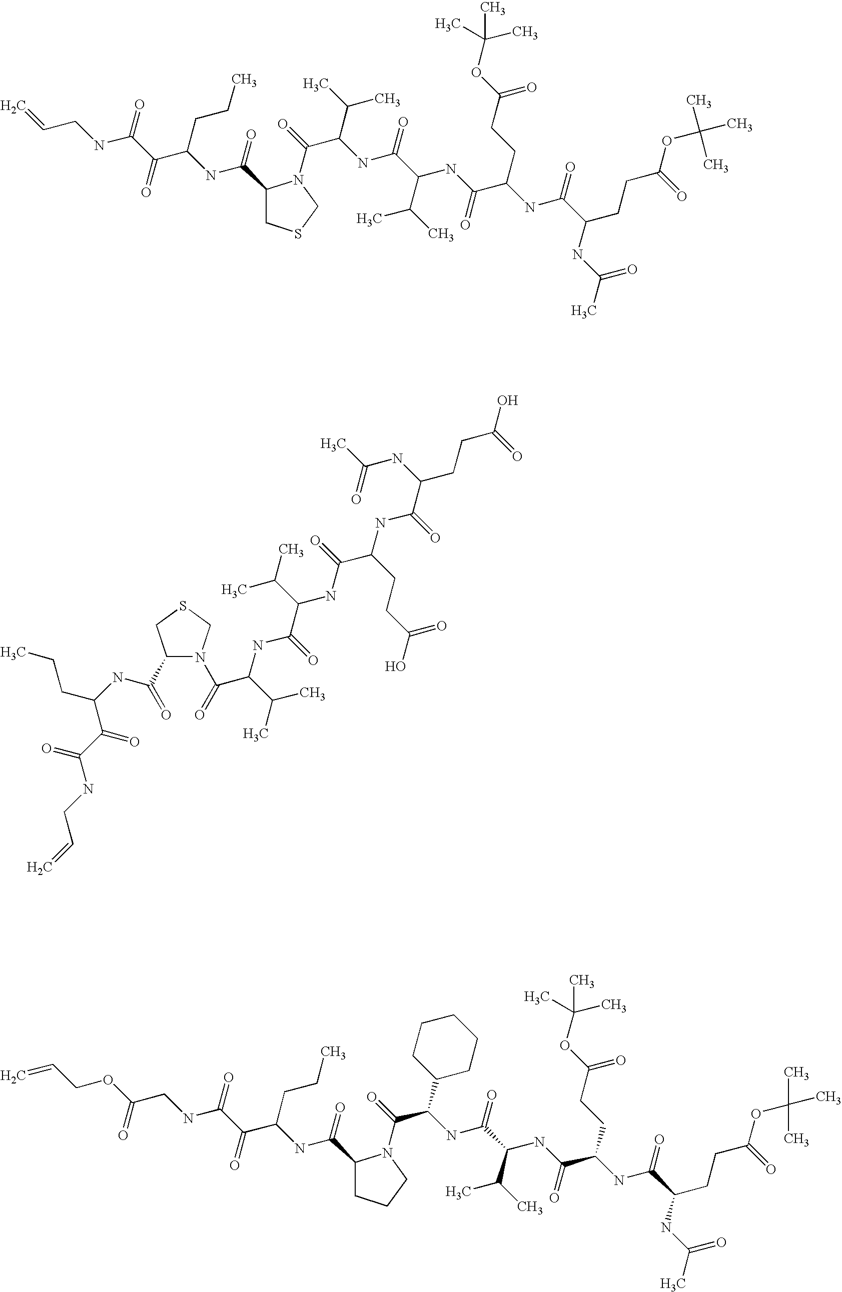 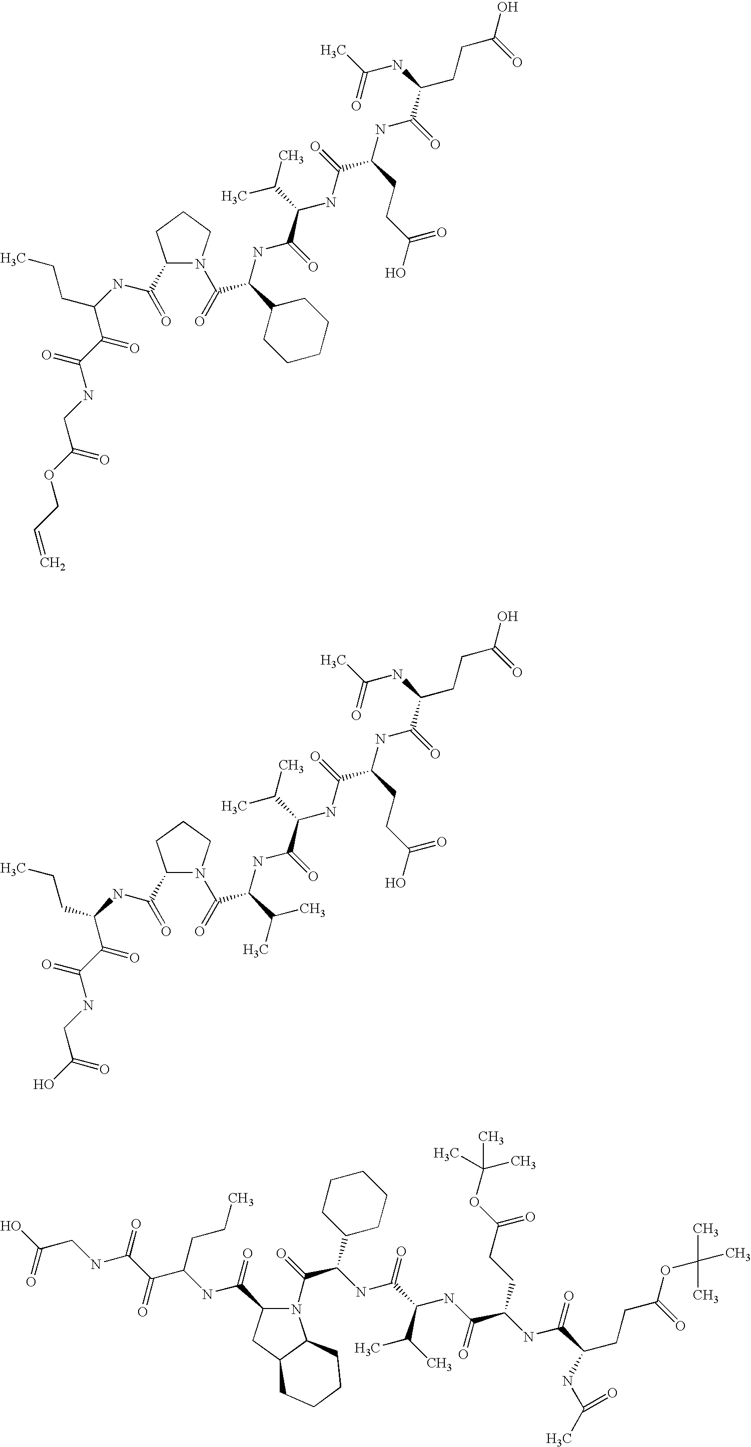 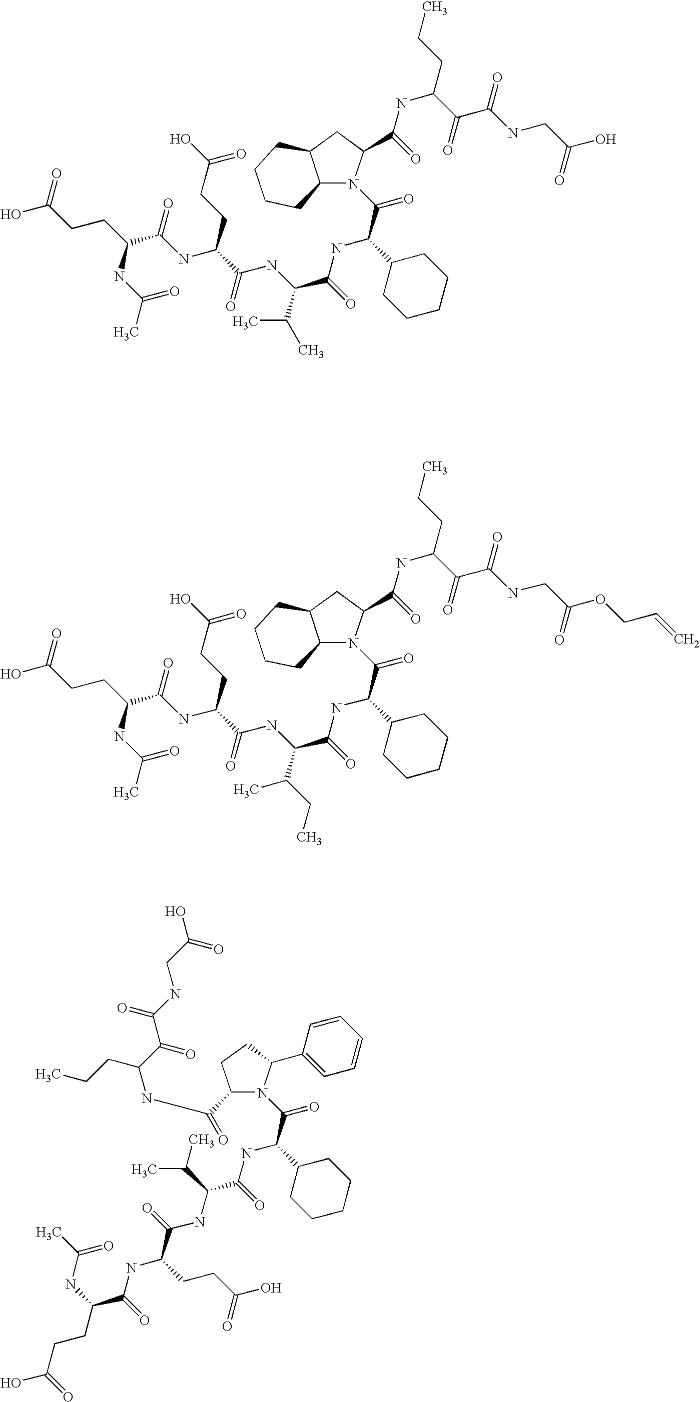 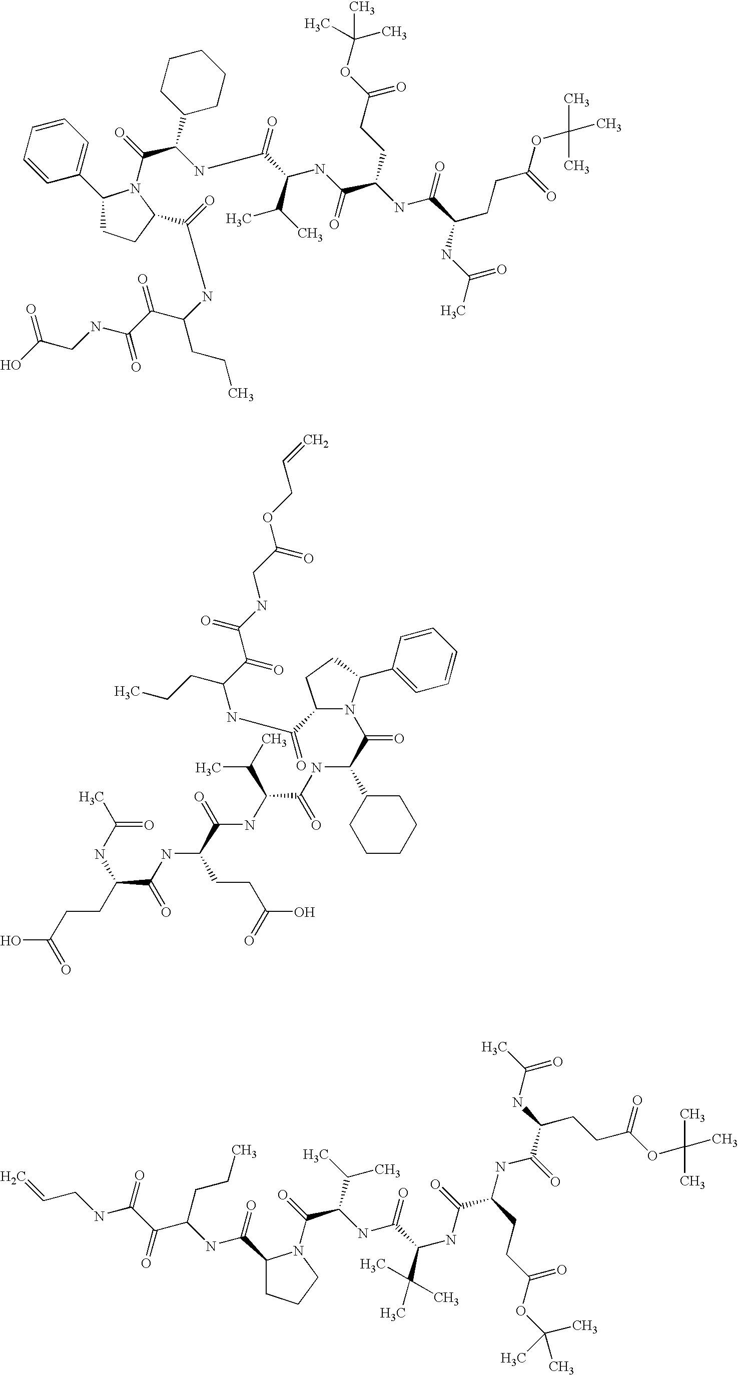 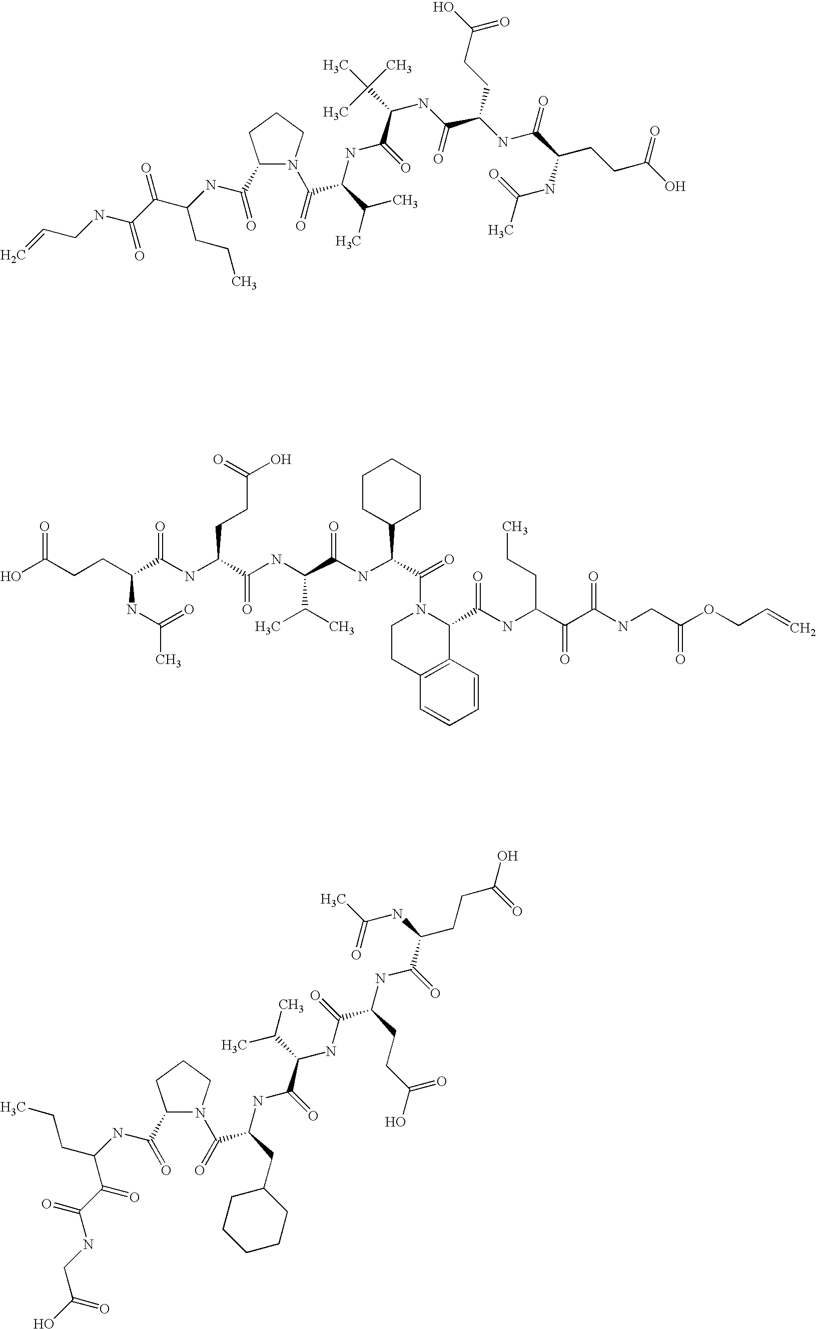 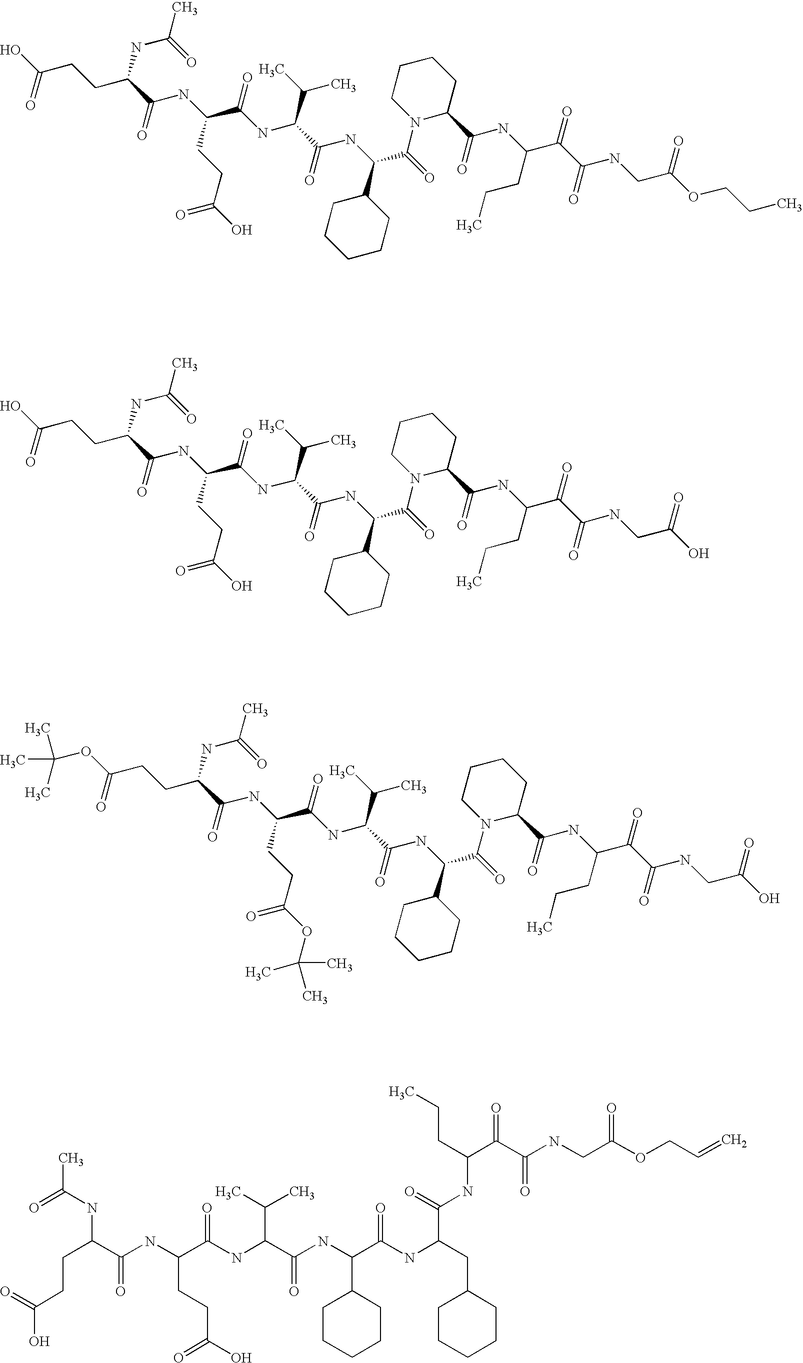 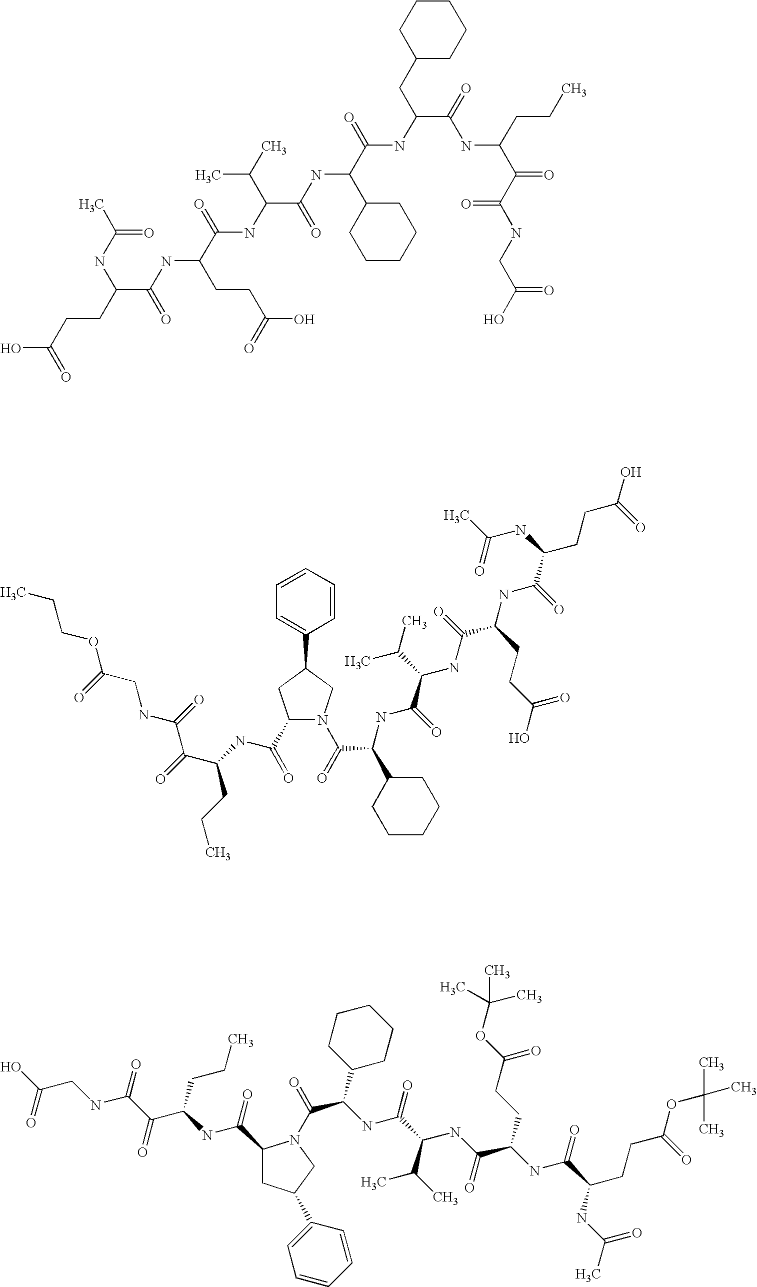 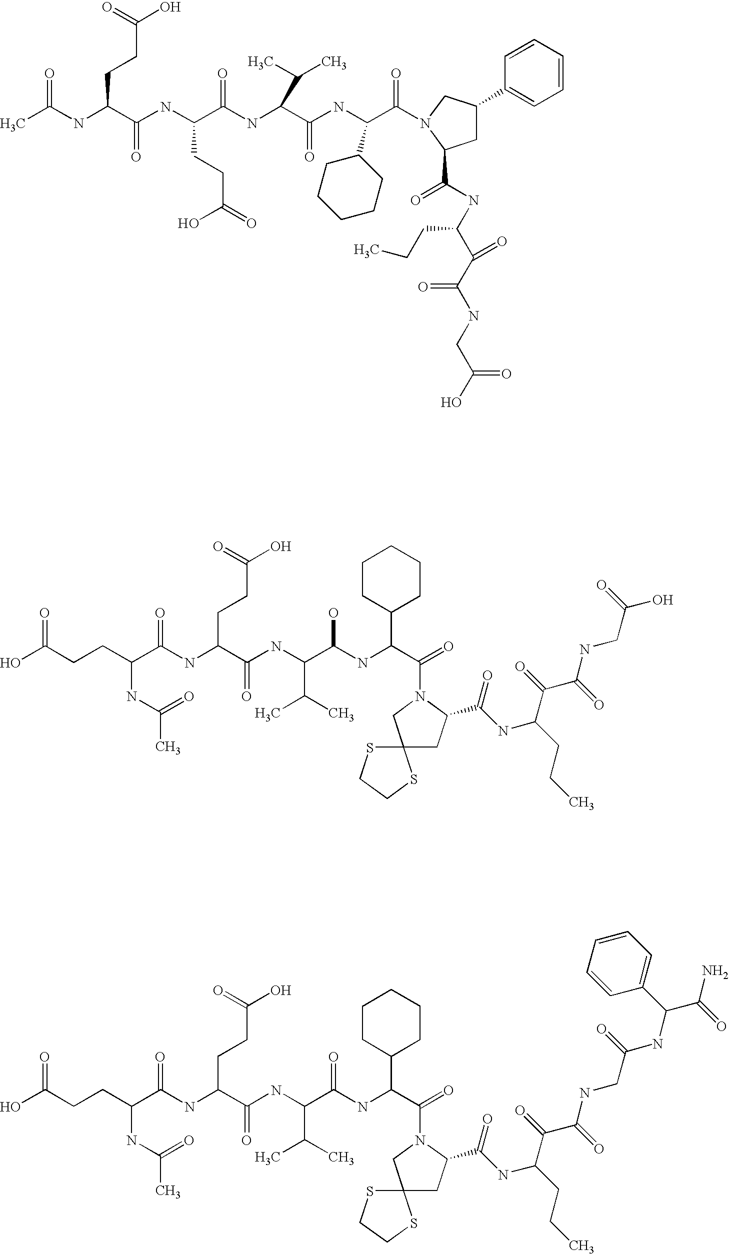 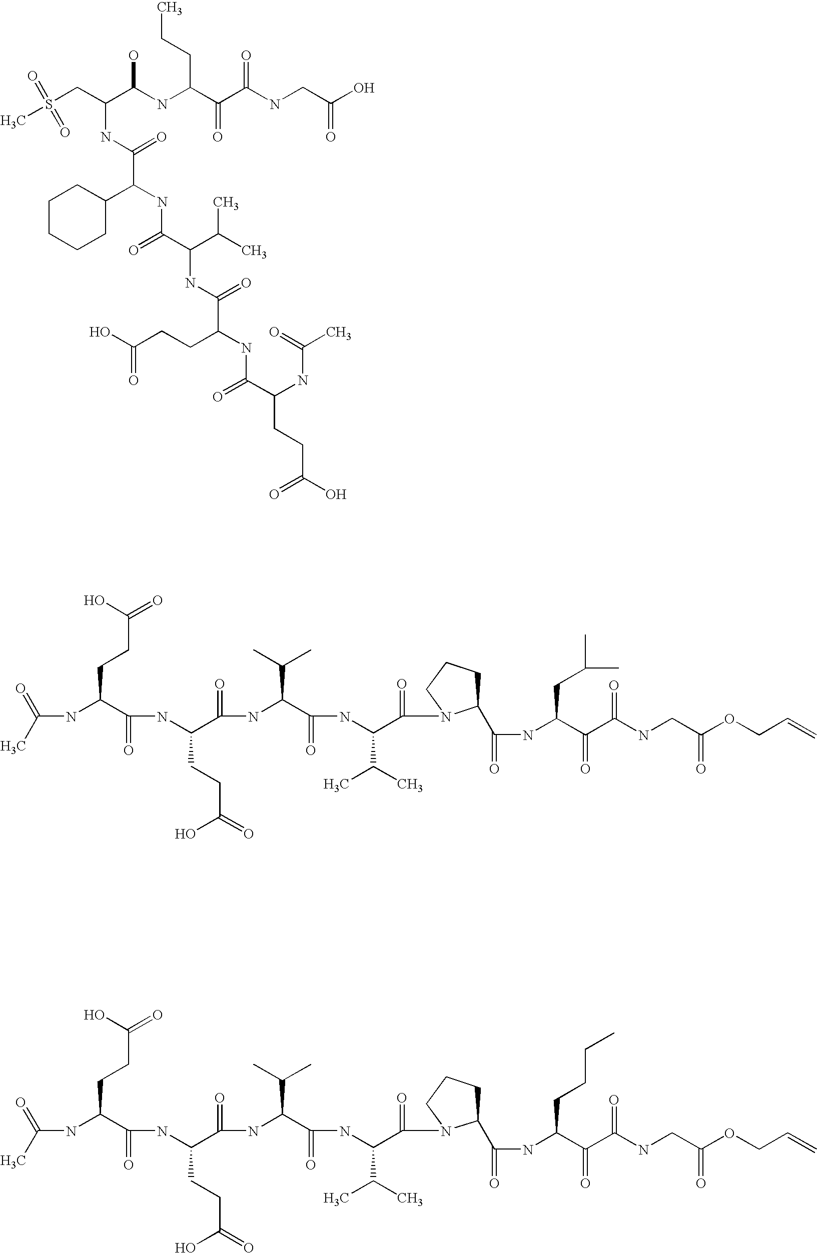 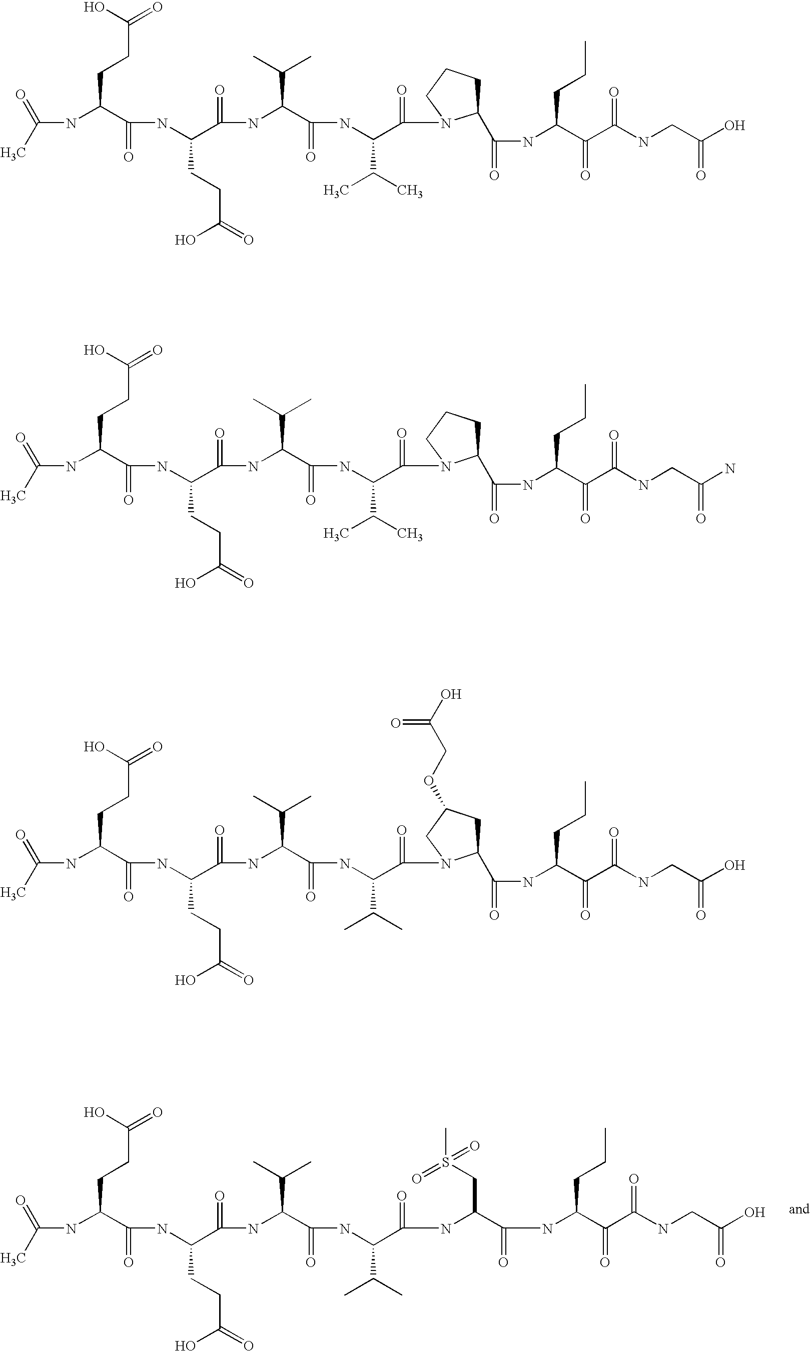 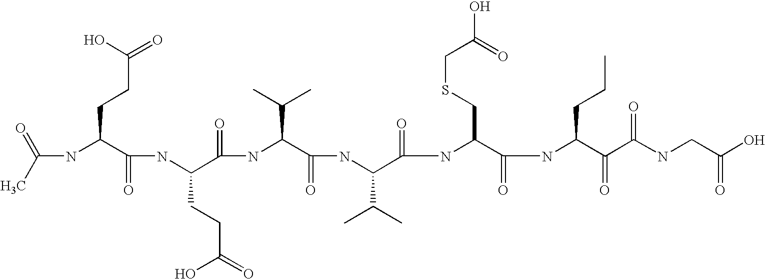
or a pharmaceutically acceptable salt, solvate or ester thereof. Other non-limiting examples include the following compounds: 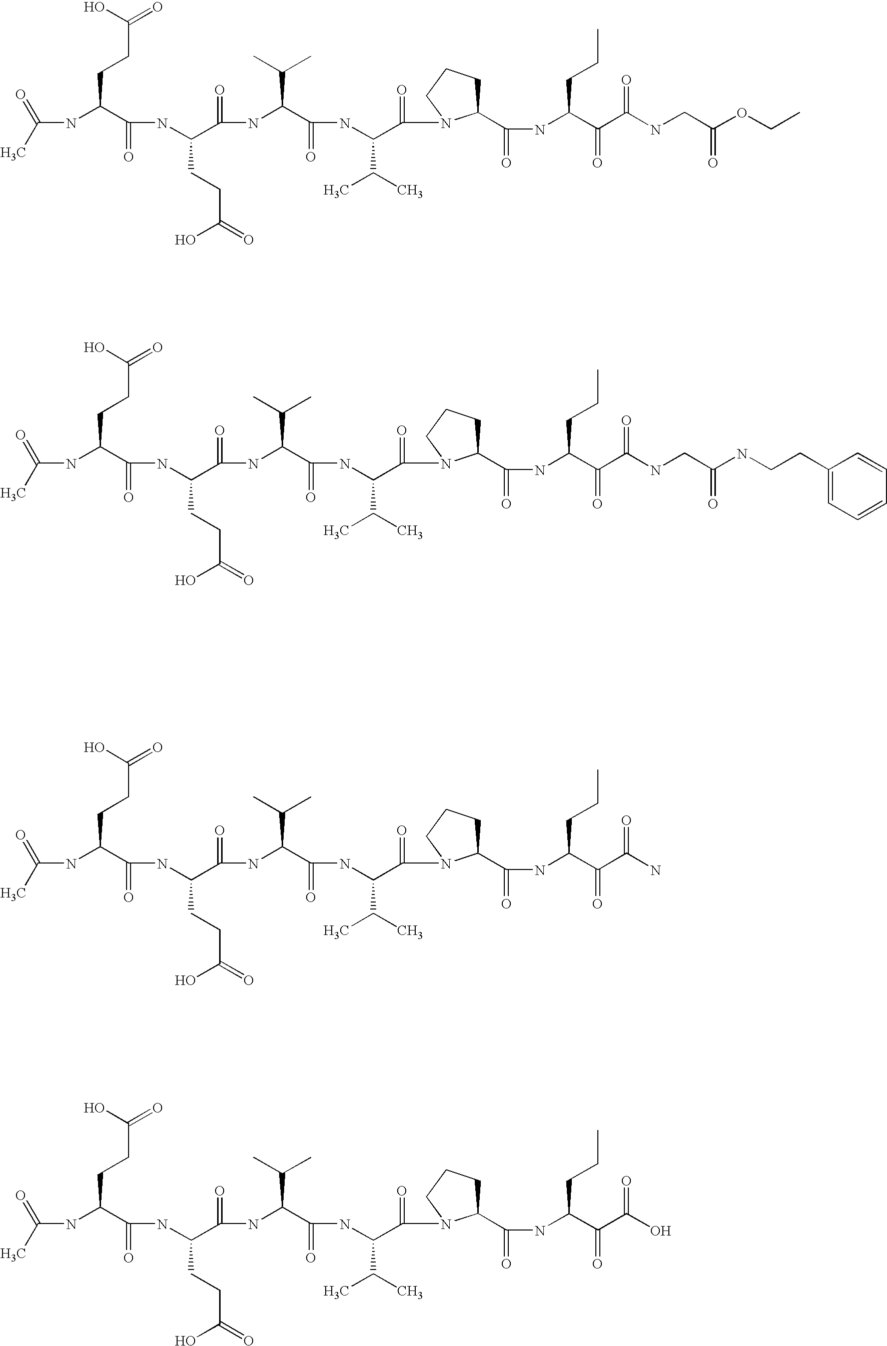 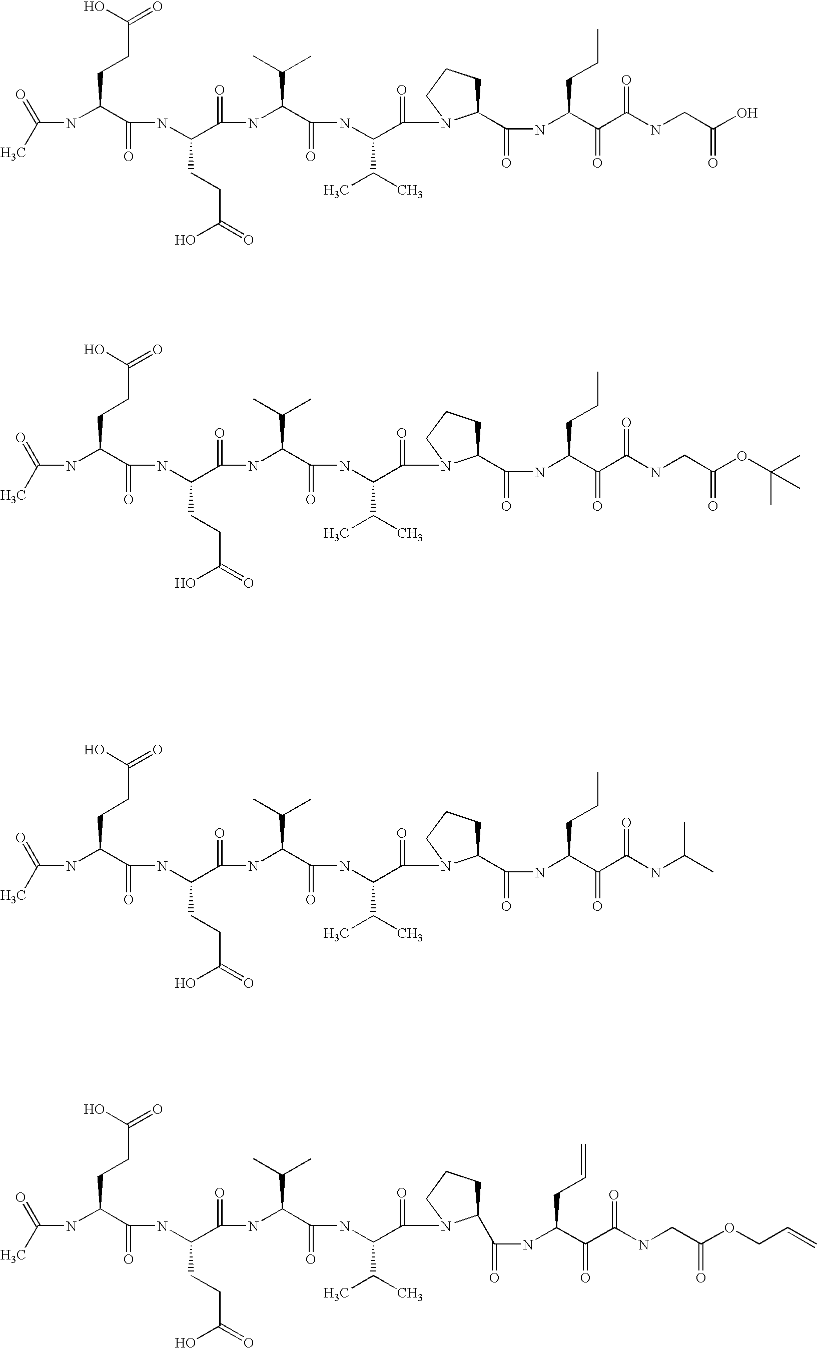 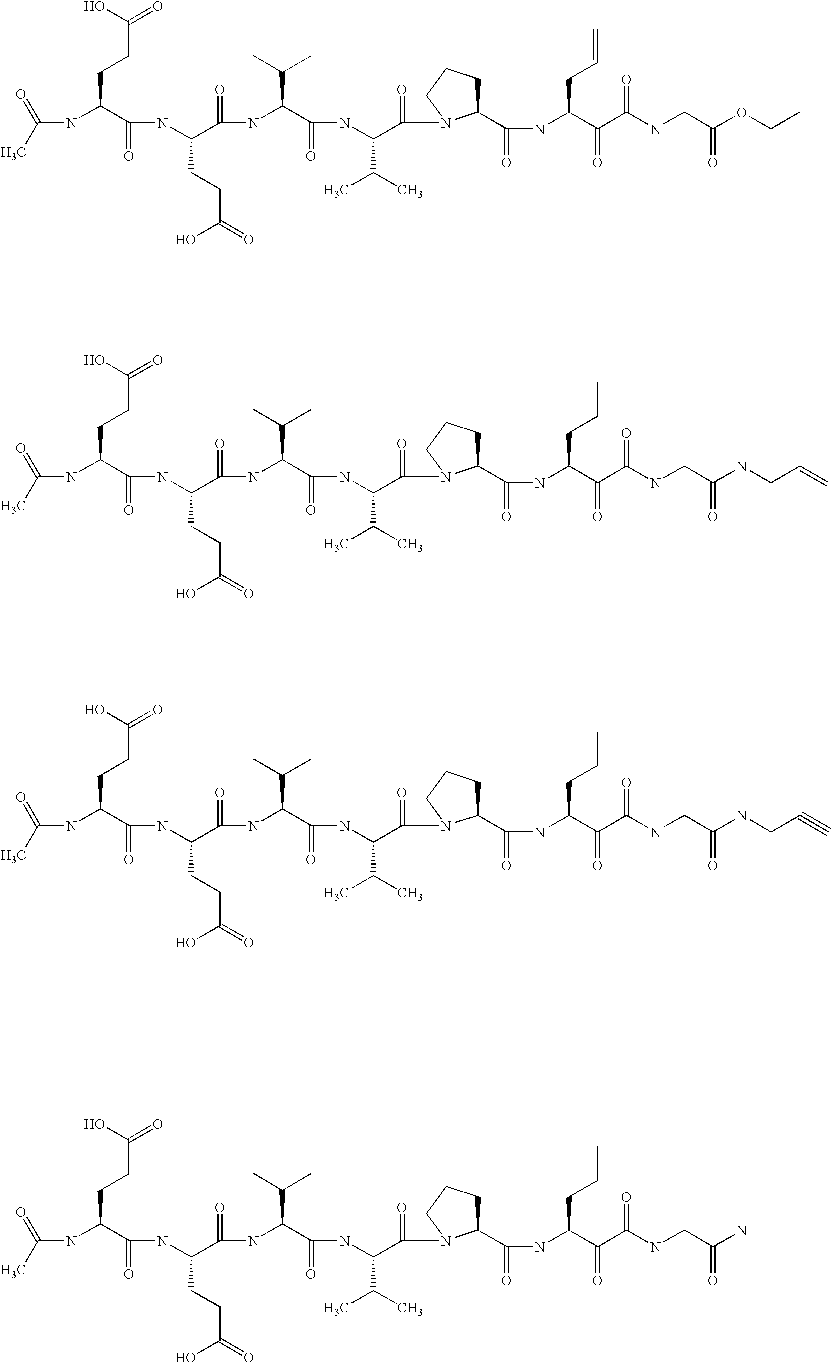 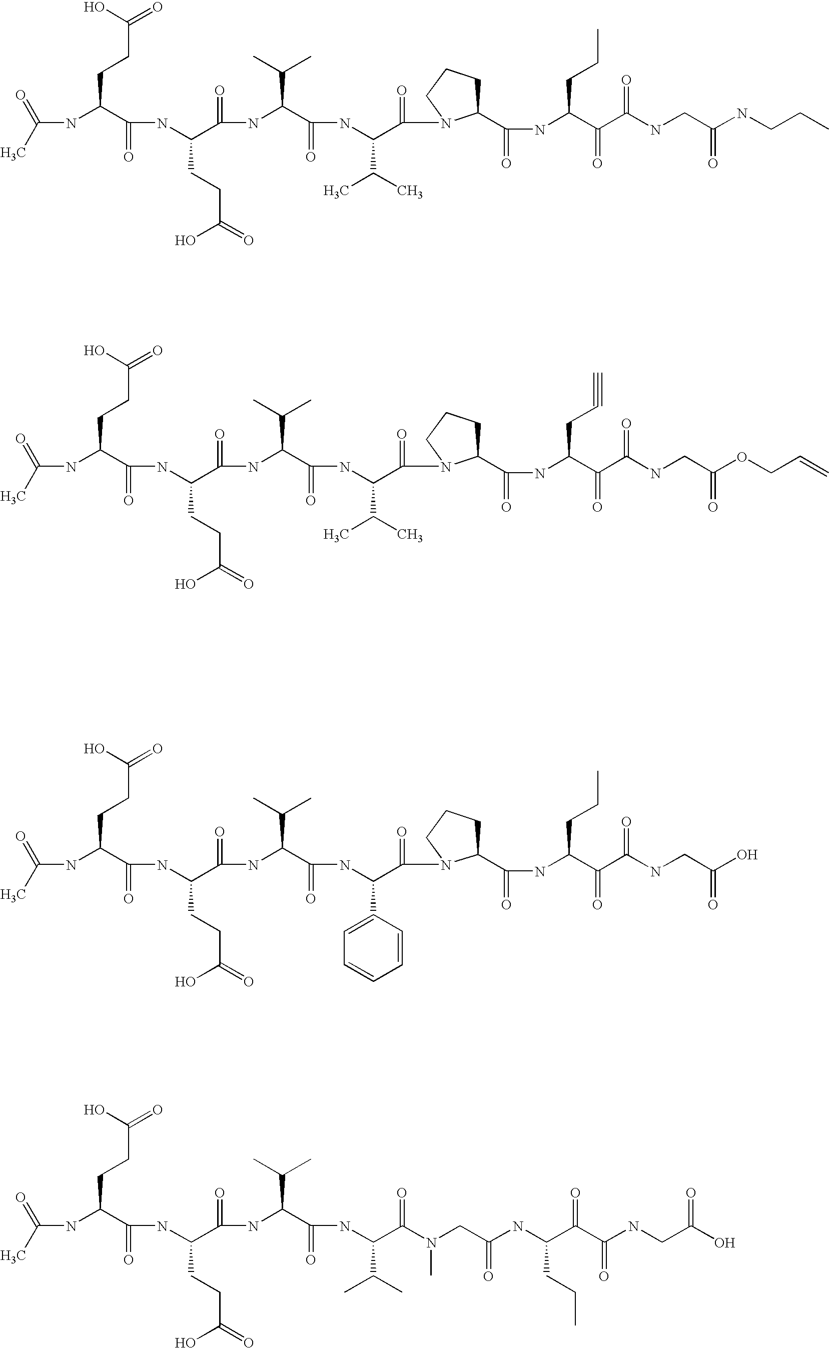 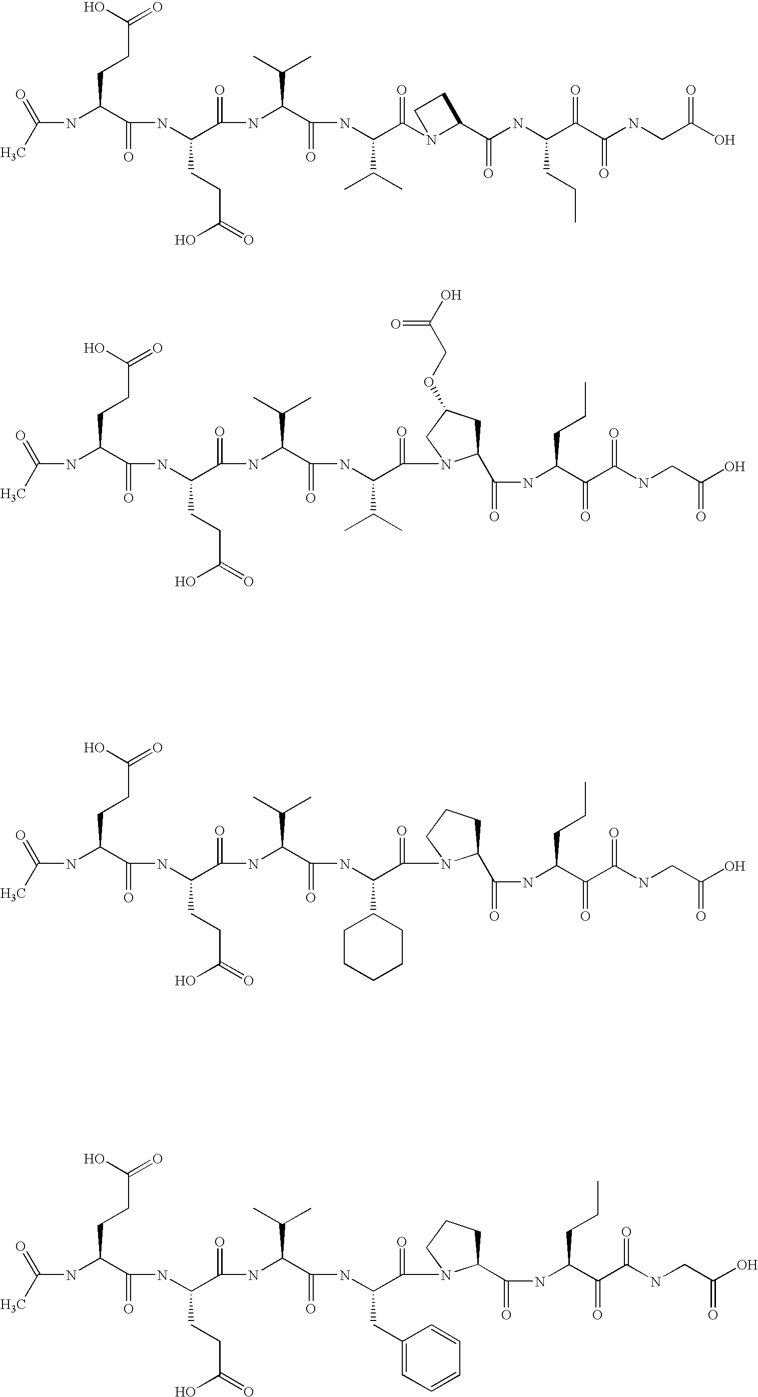 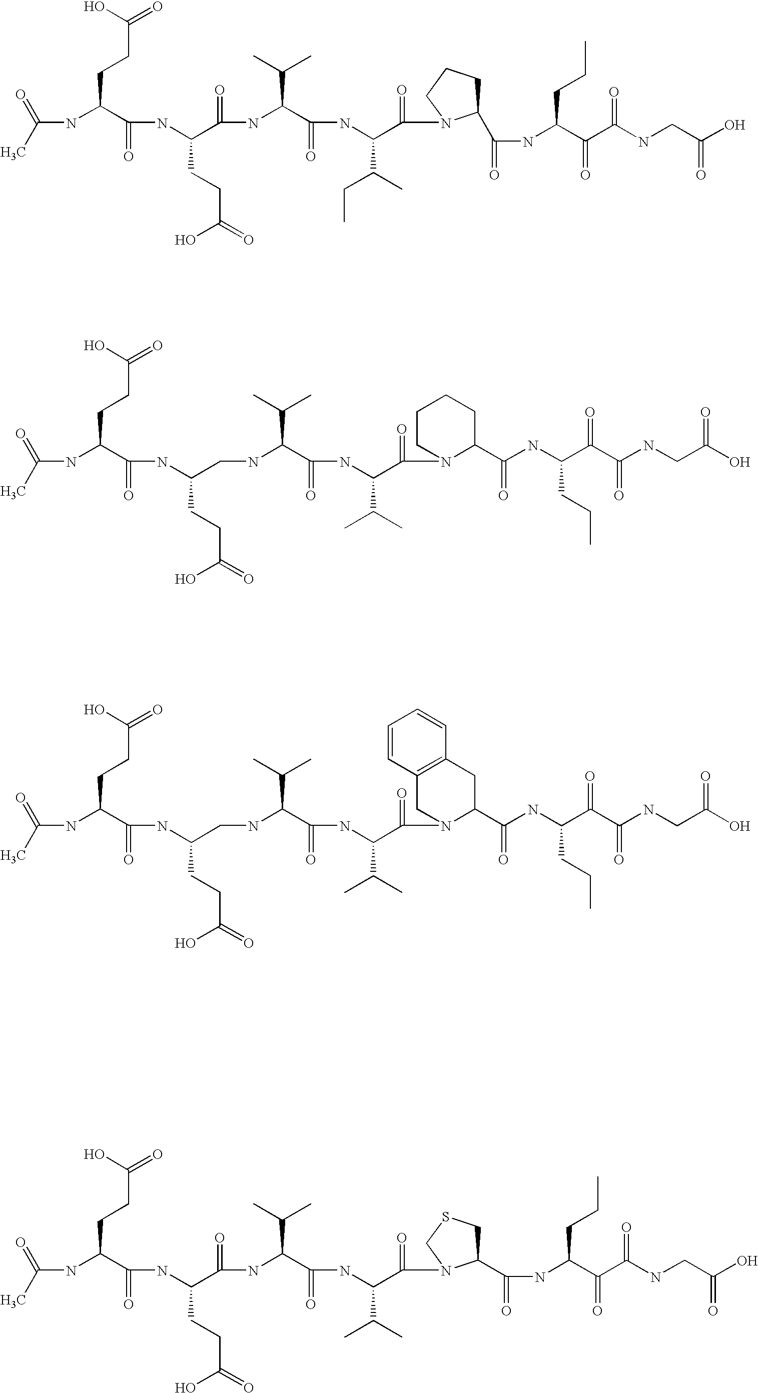 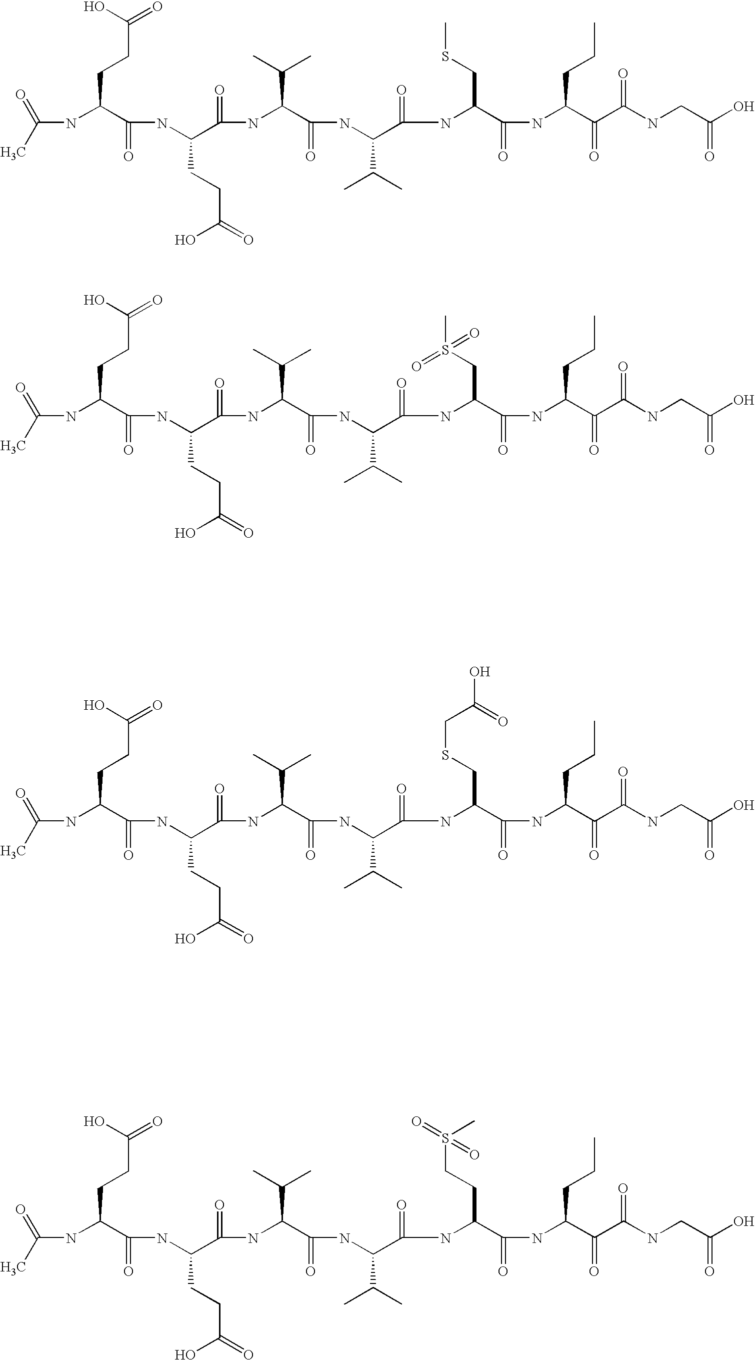 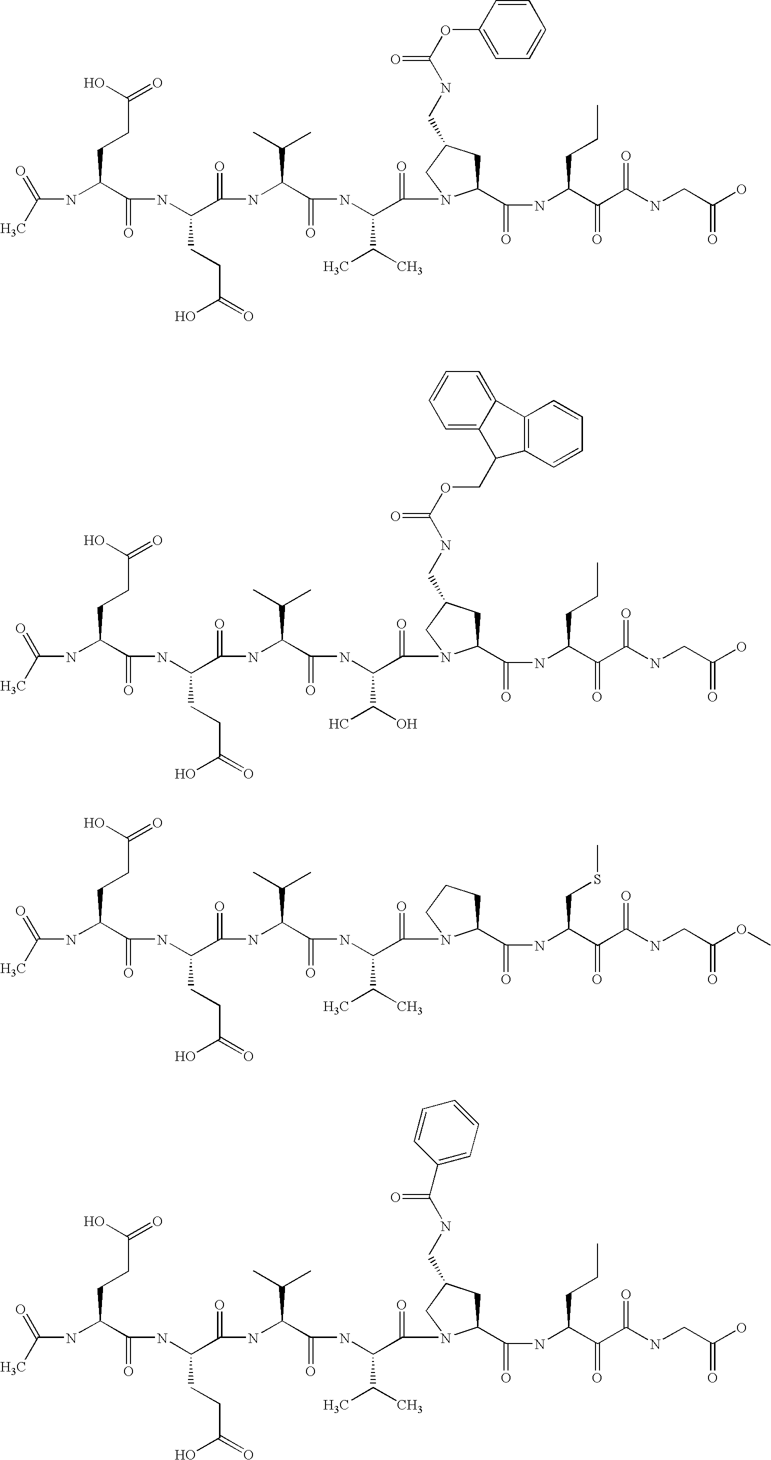 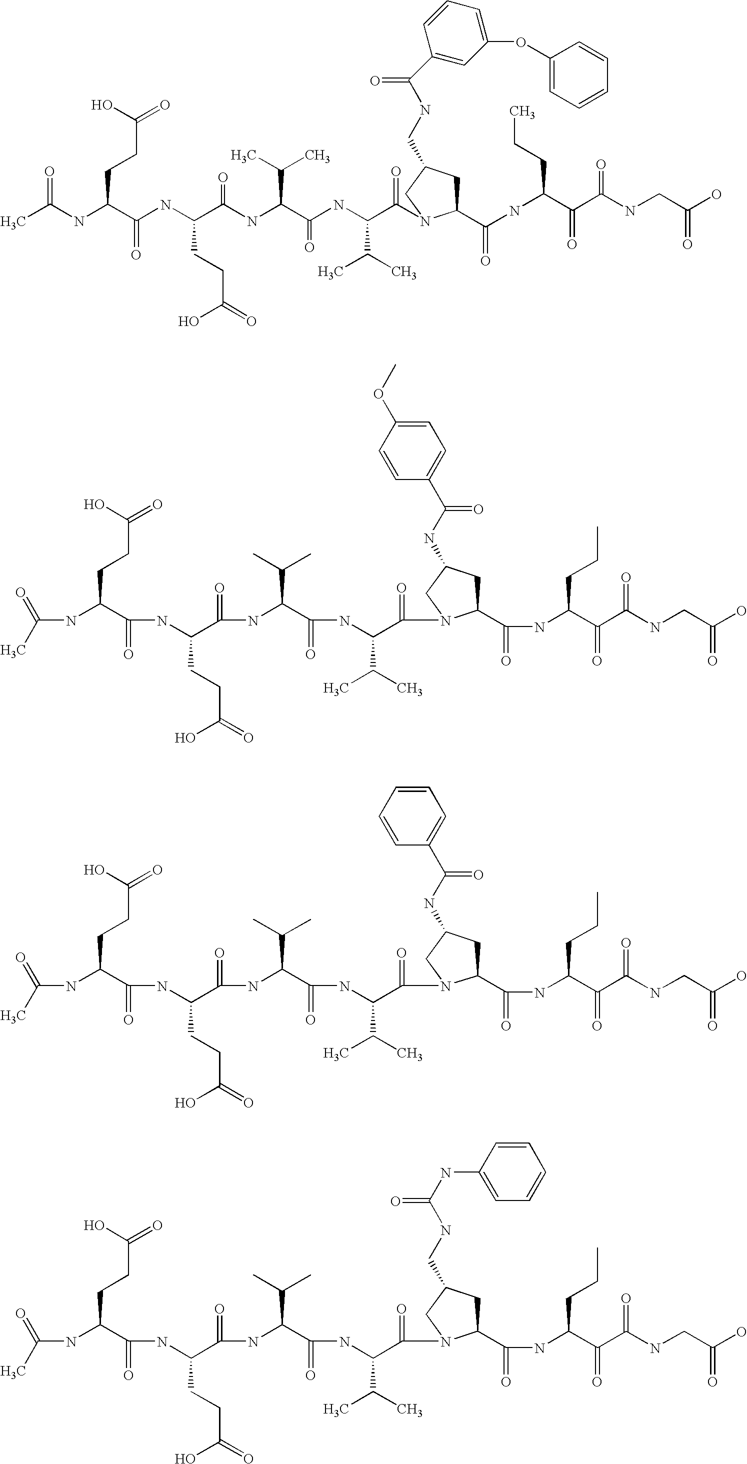 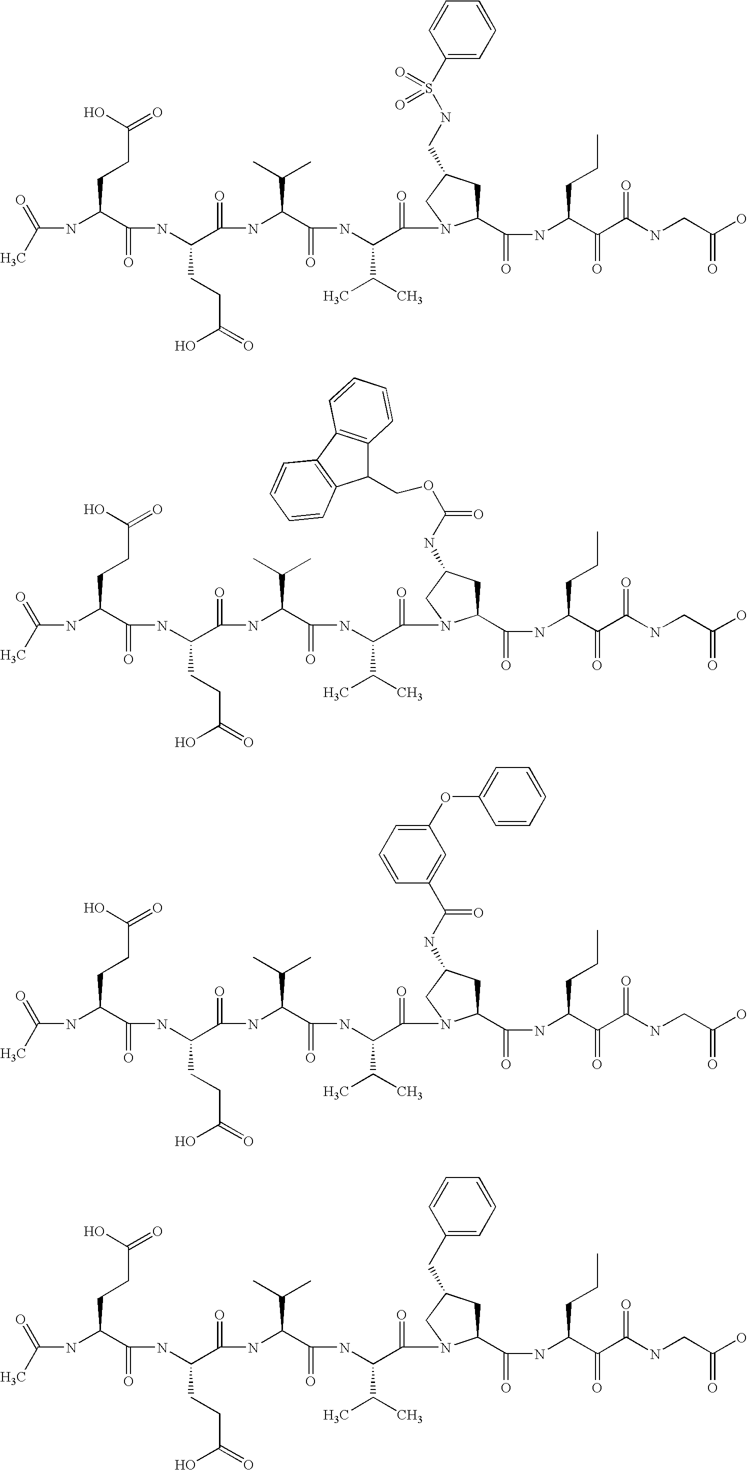 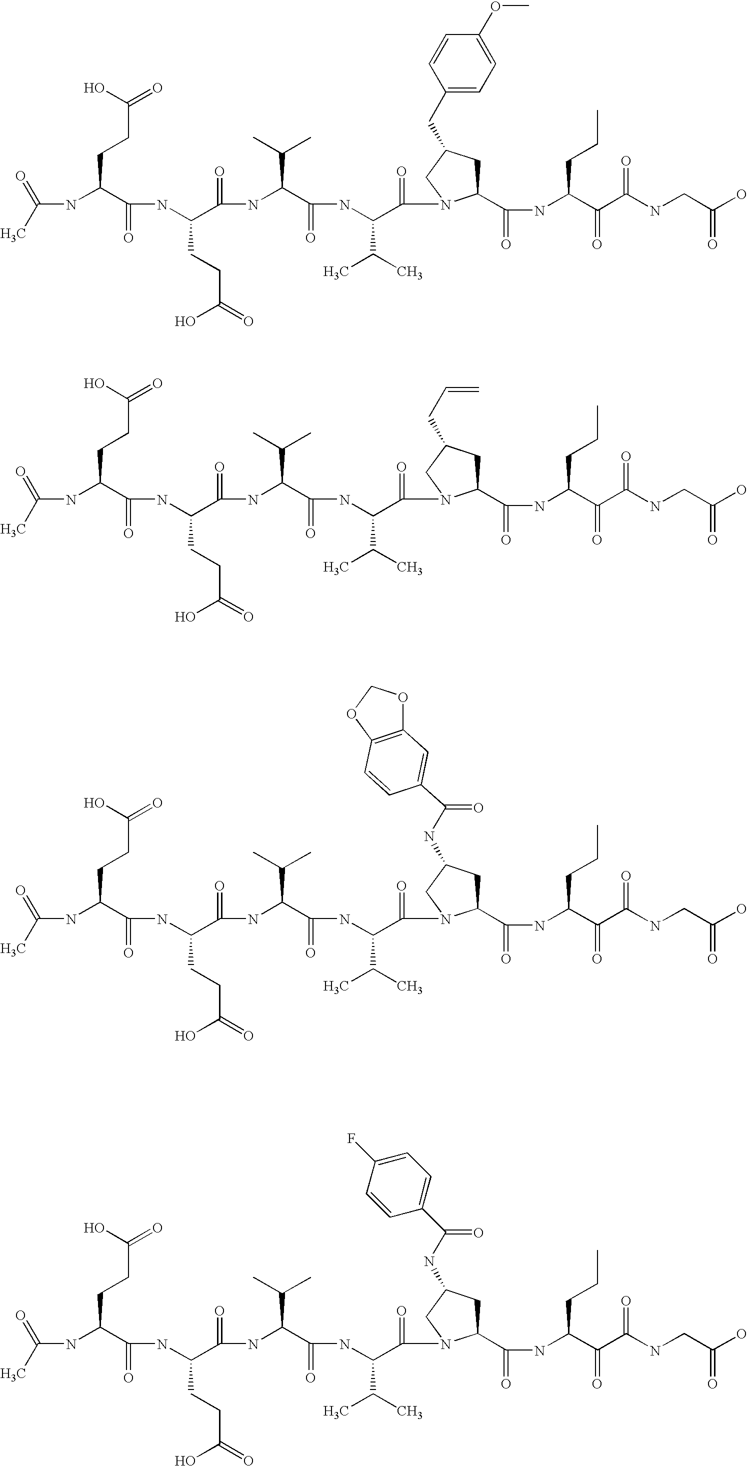 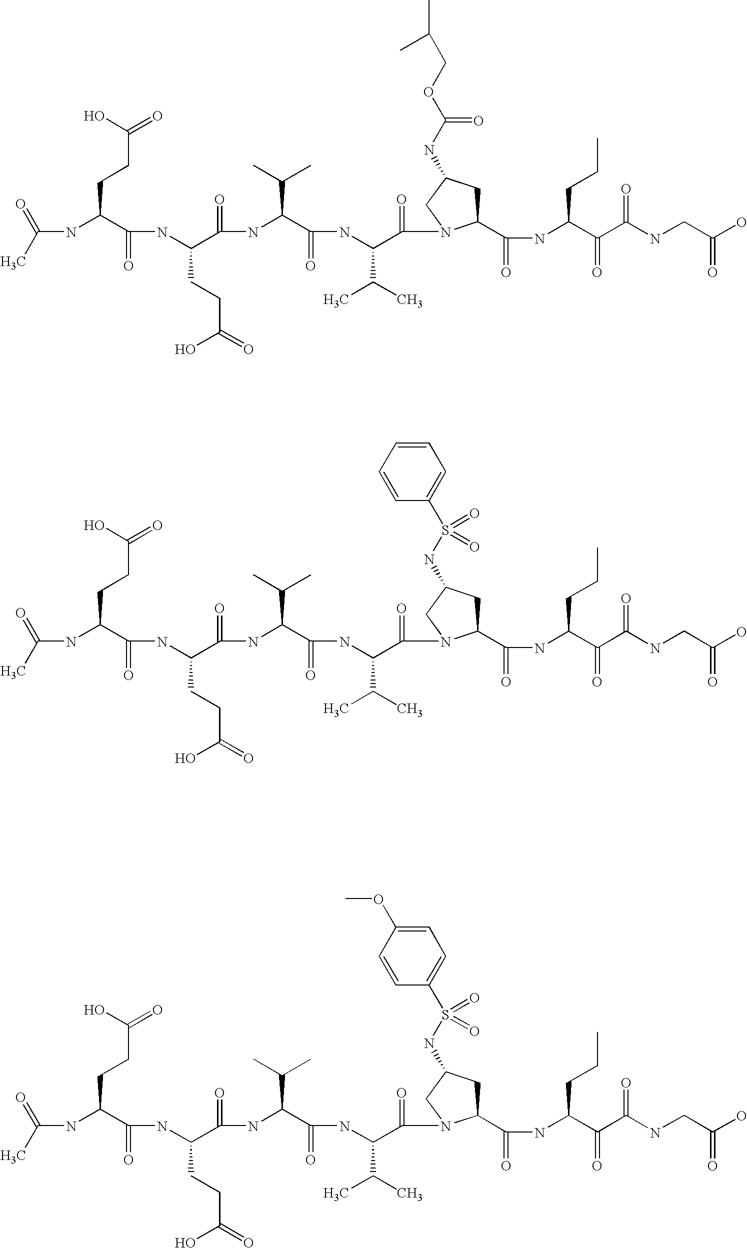 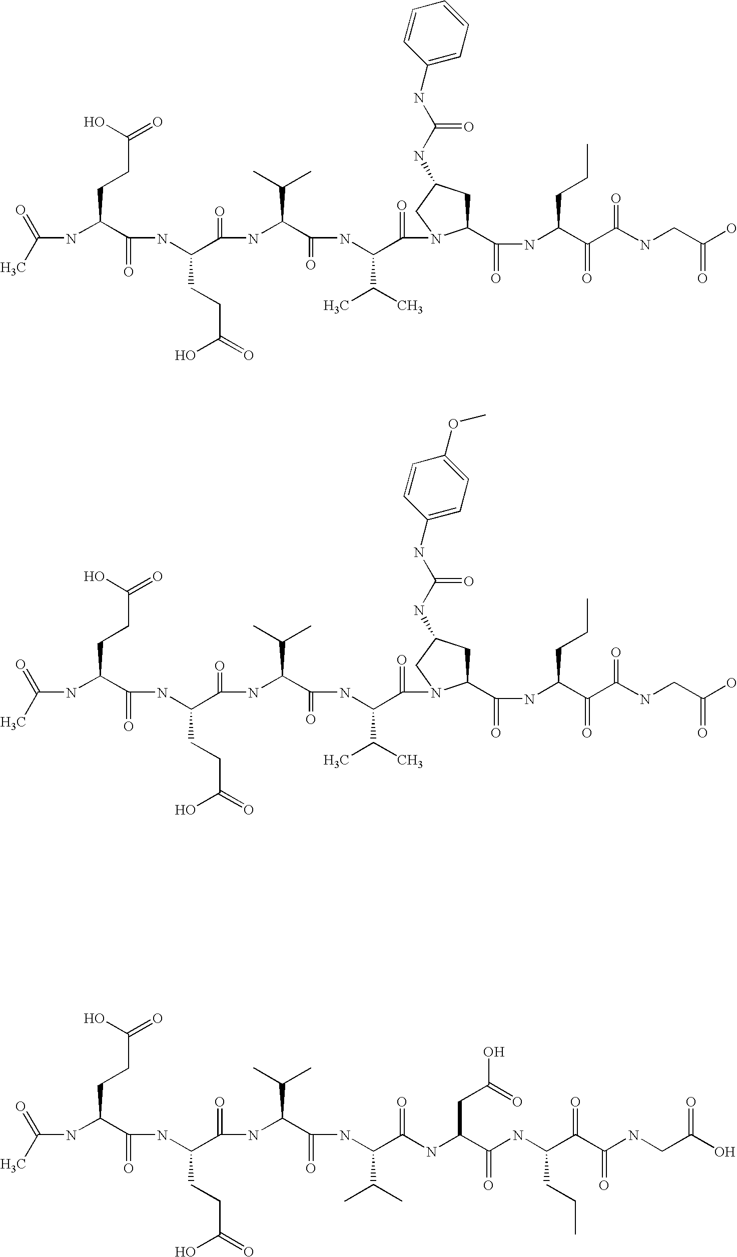 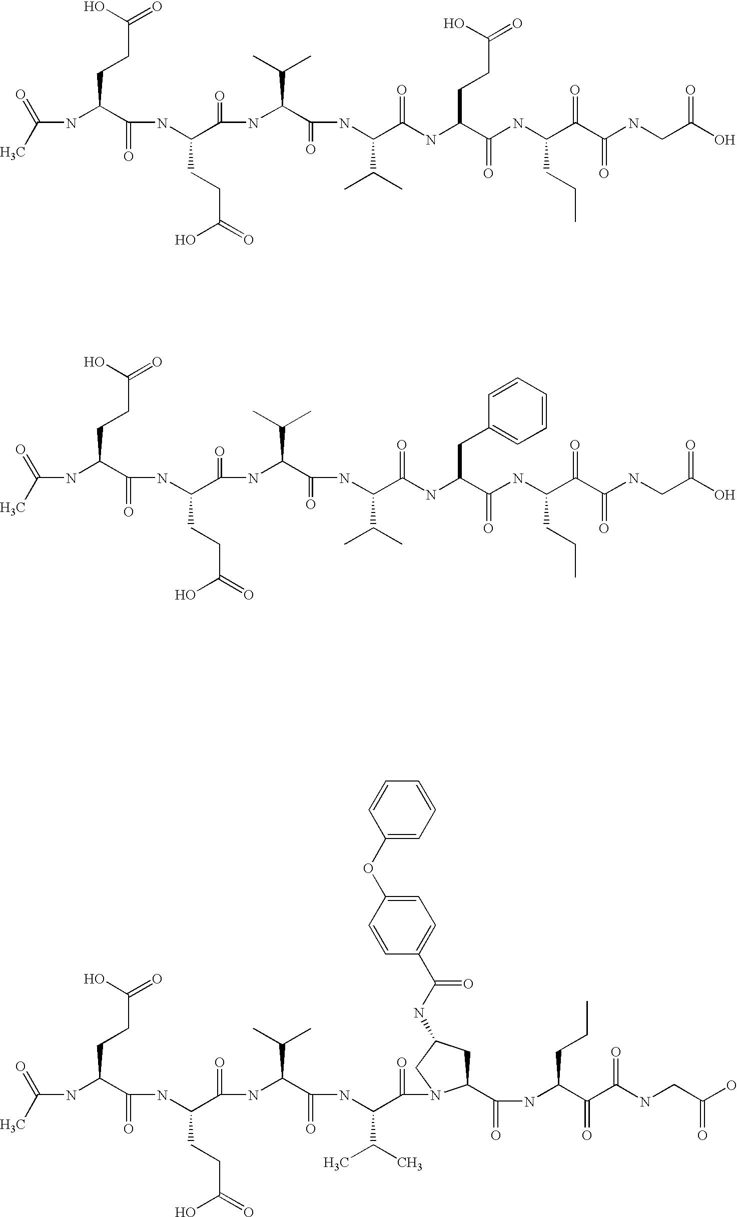 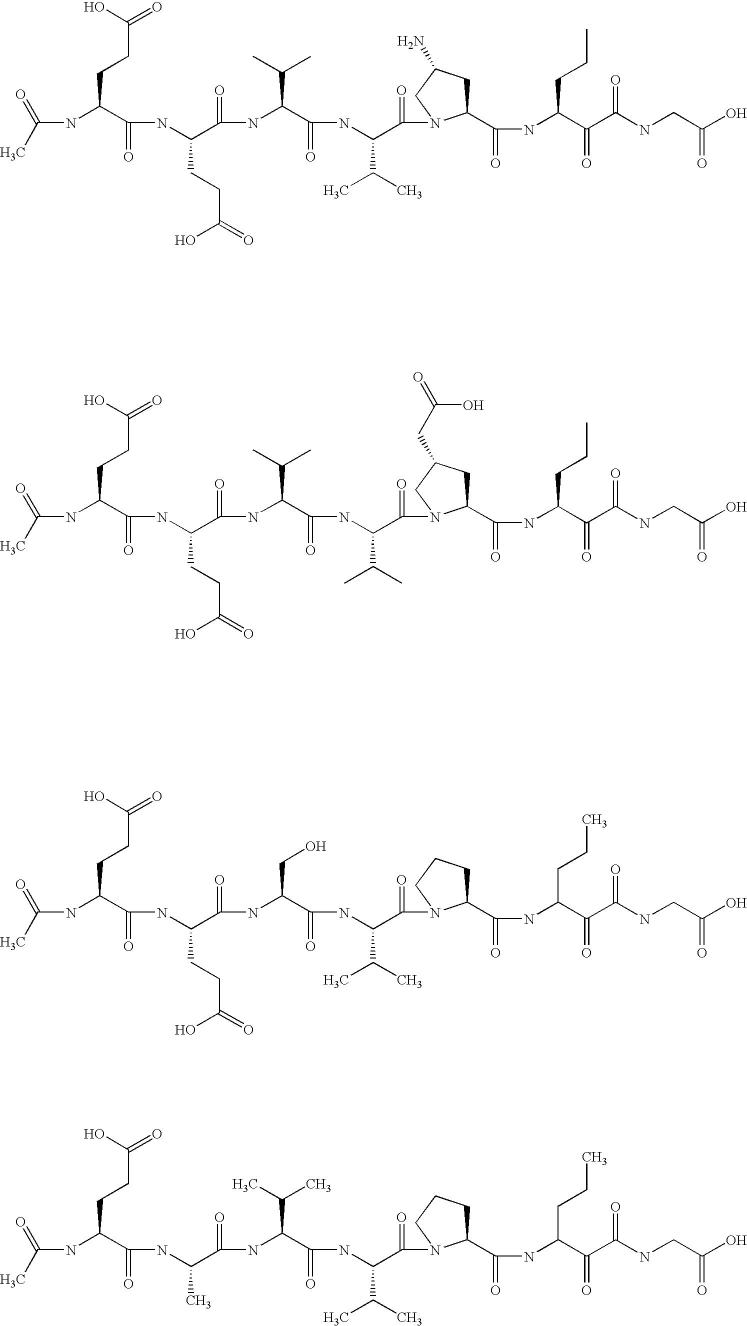 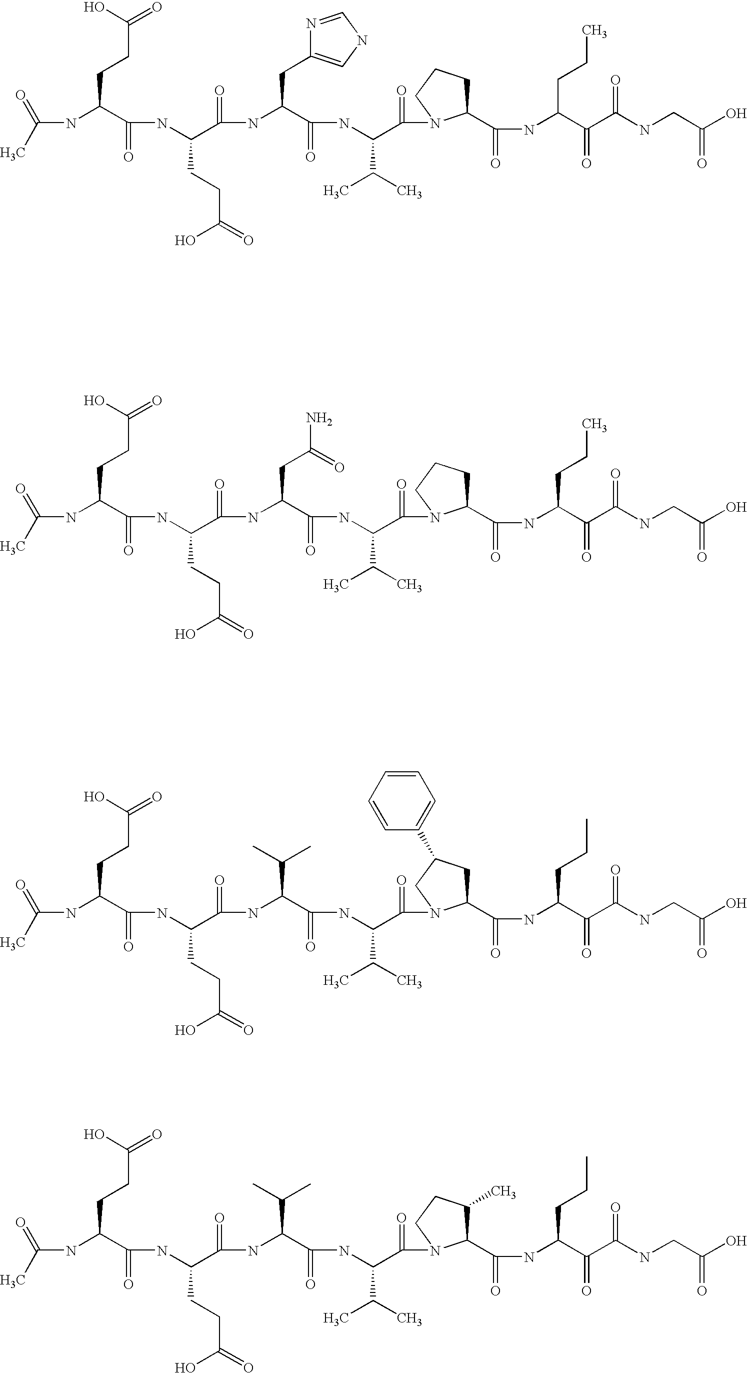 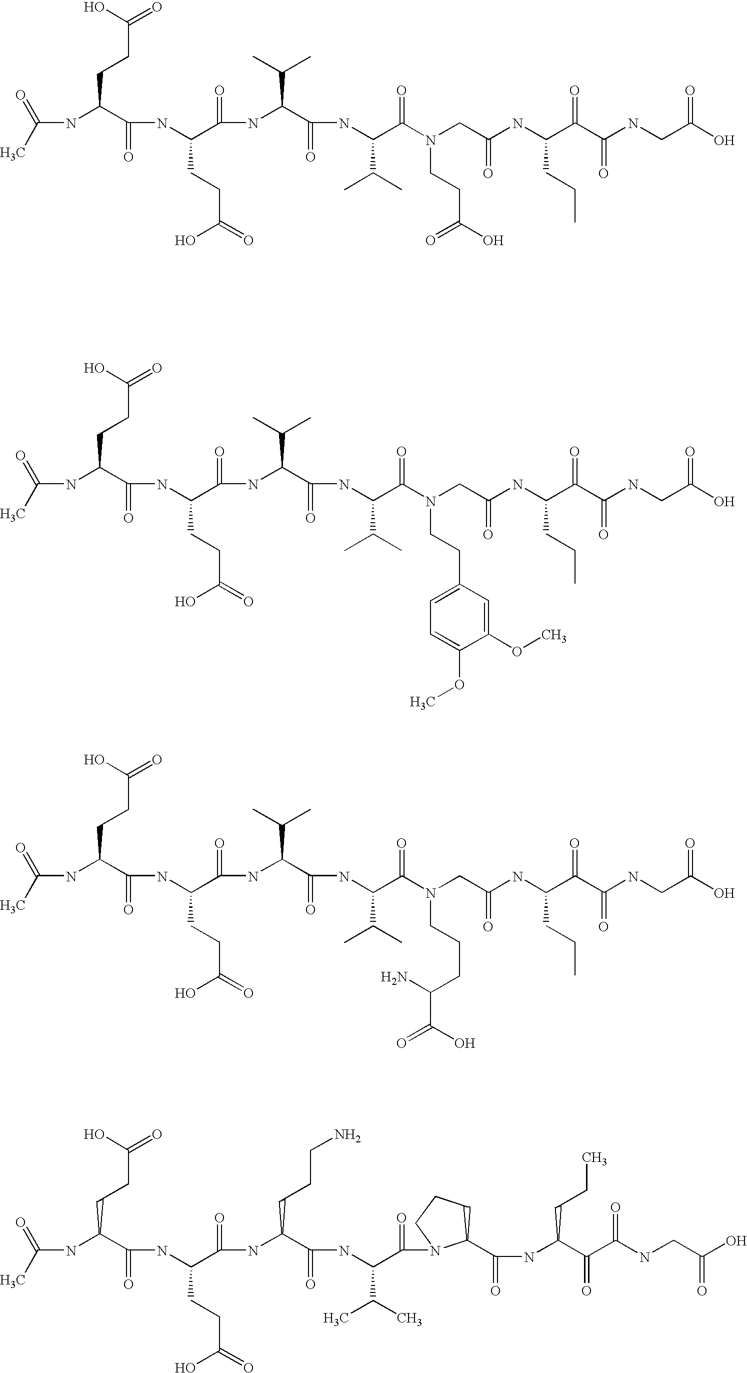 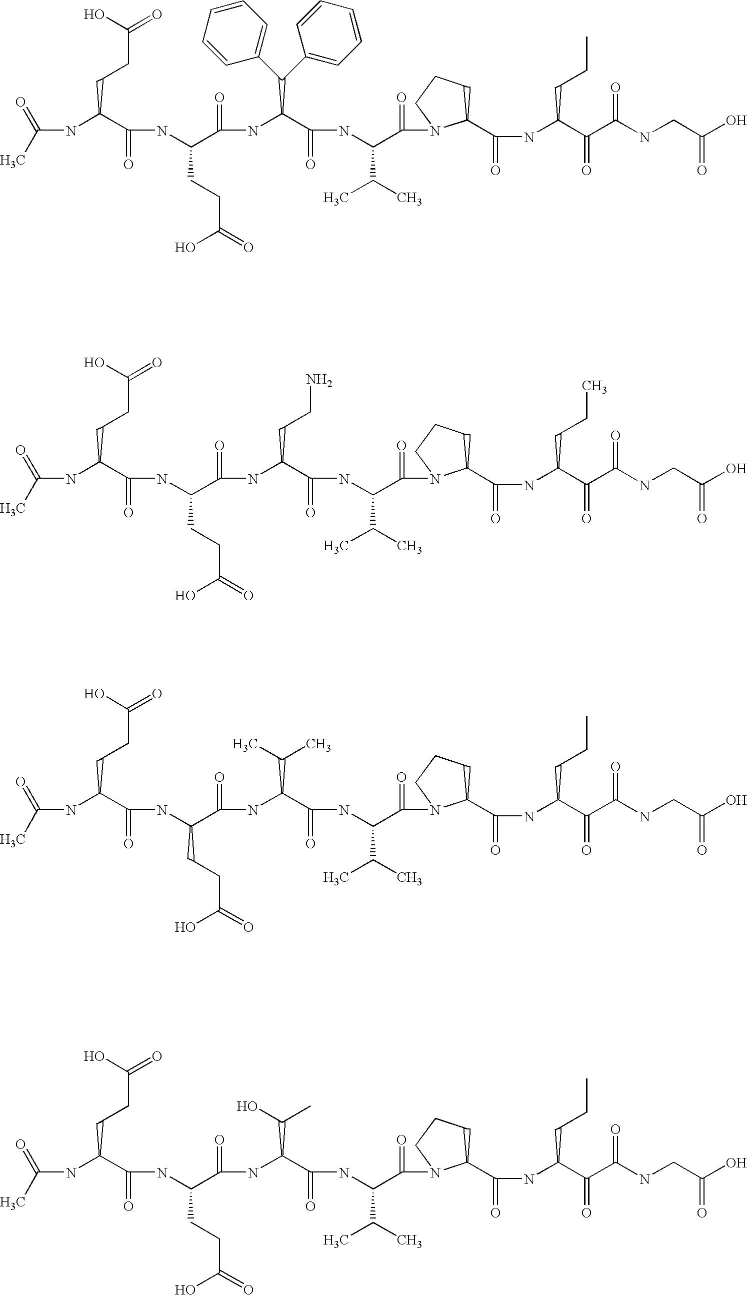 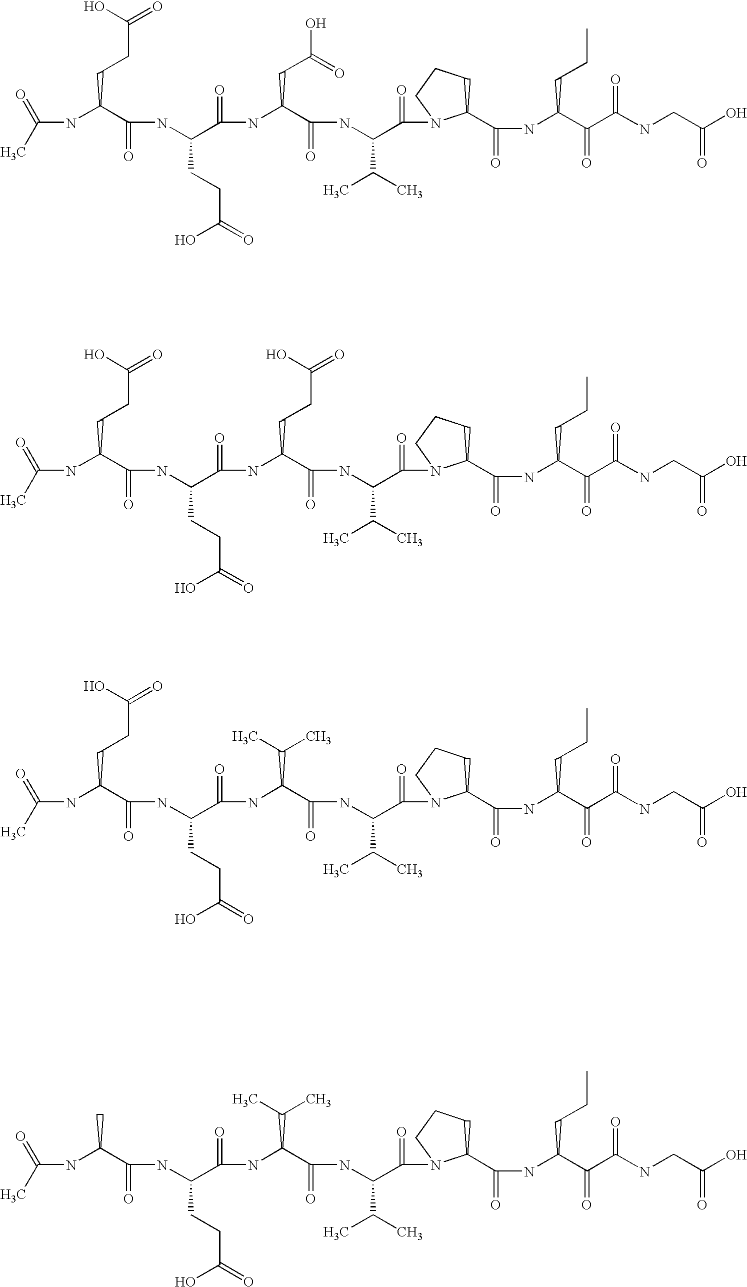 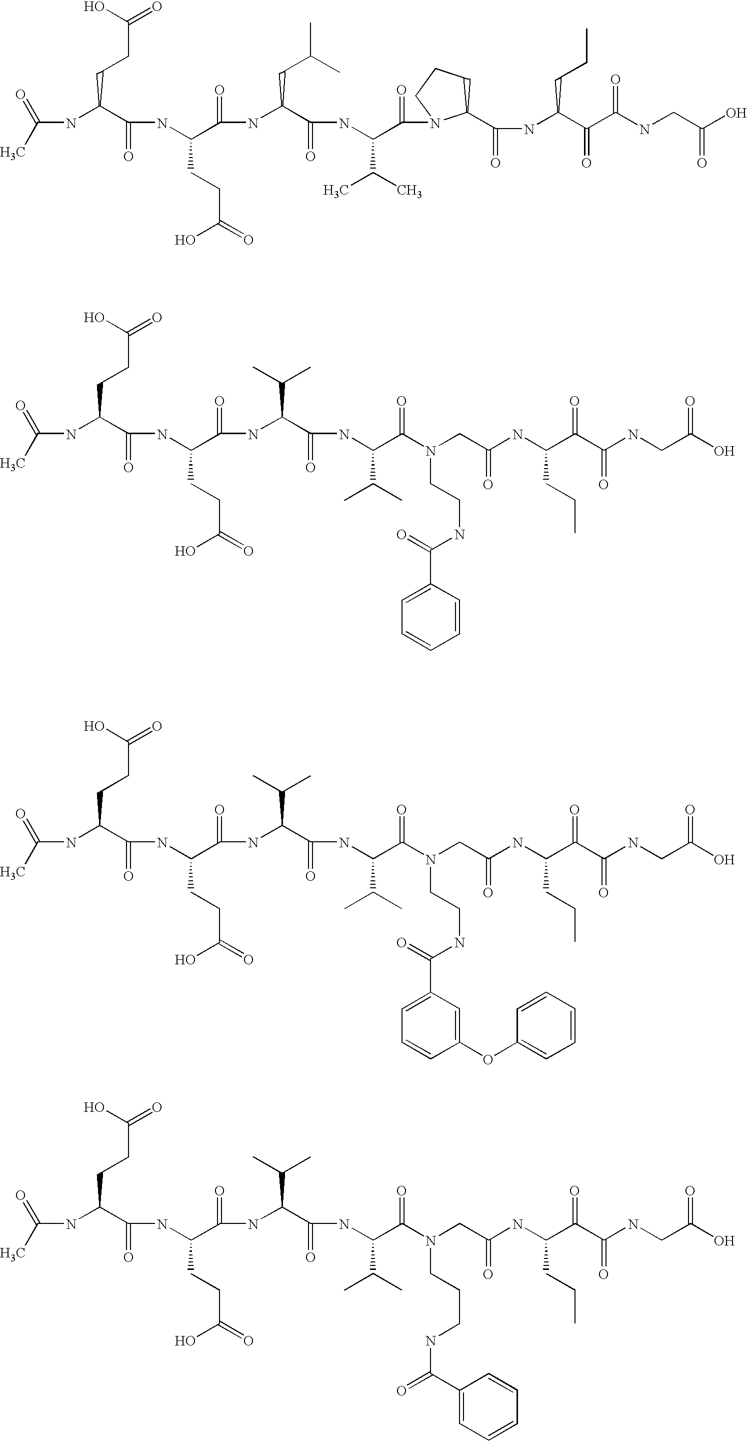 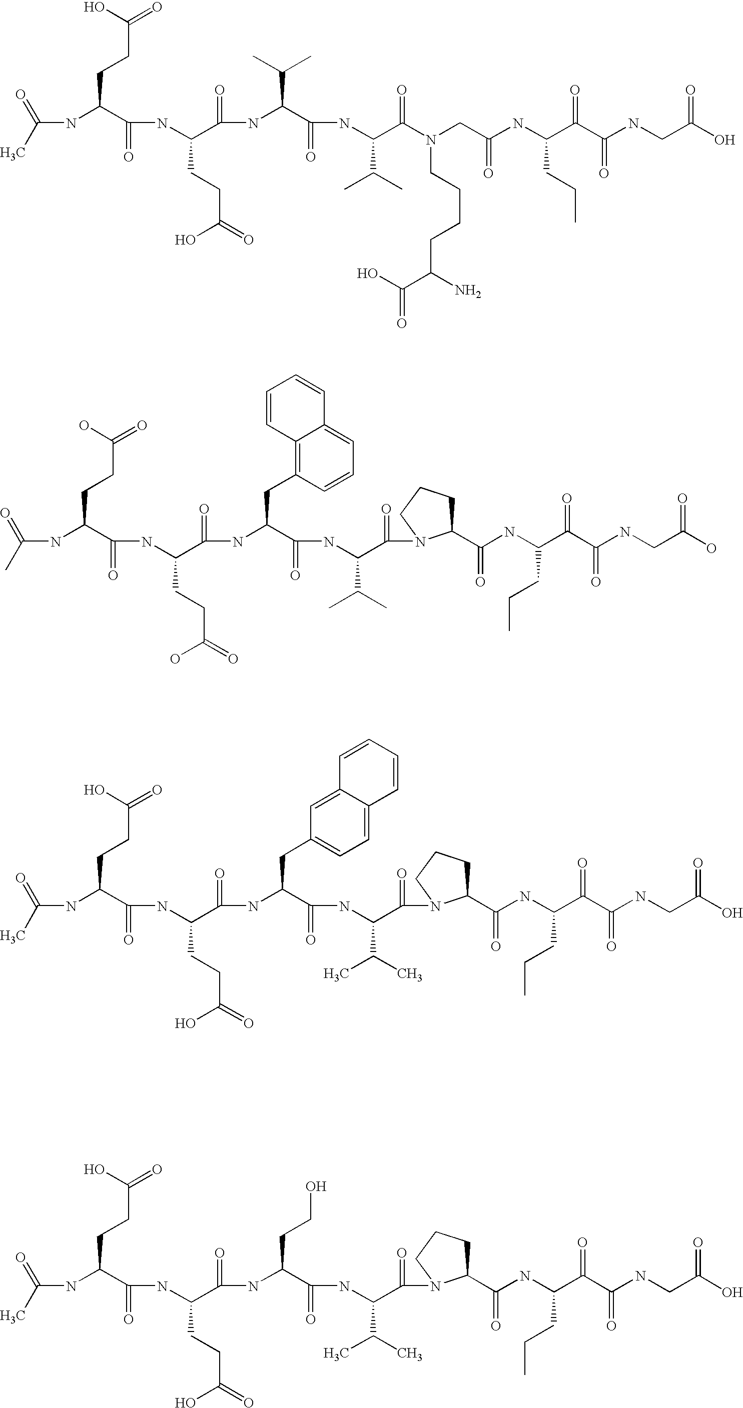 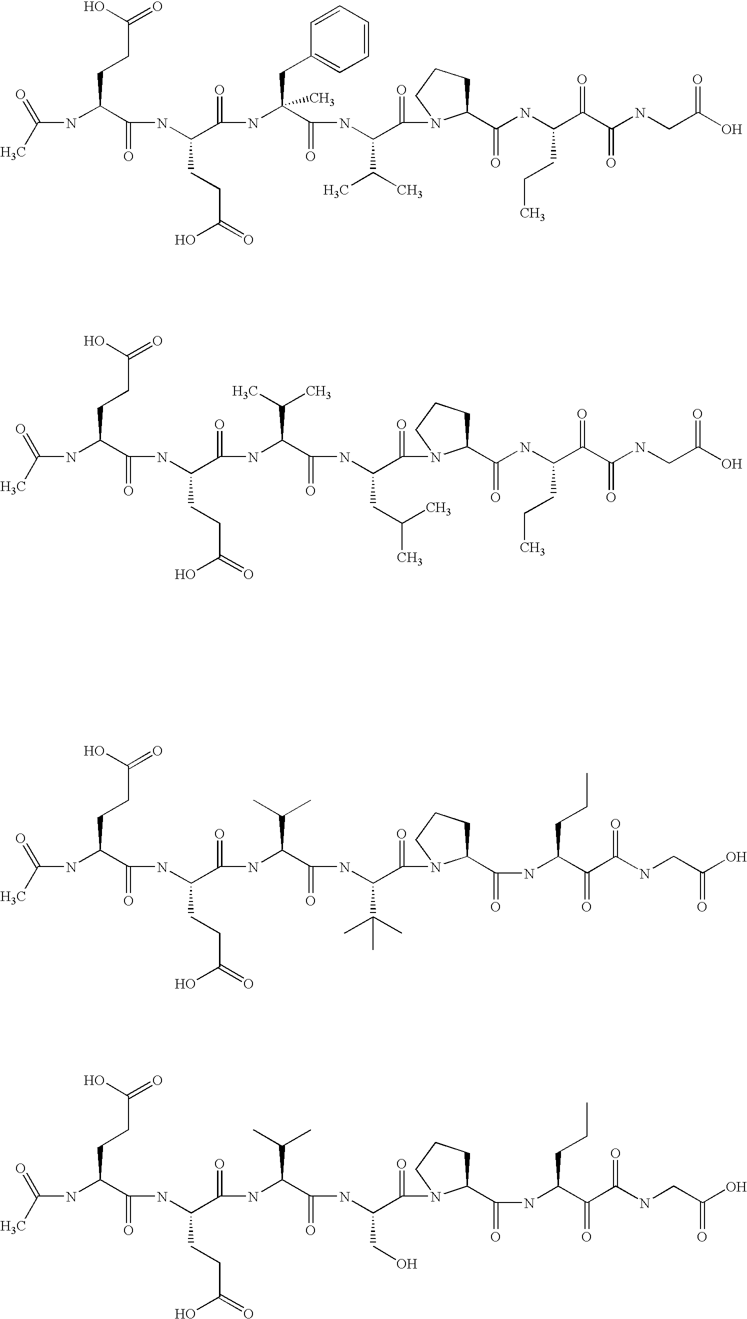 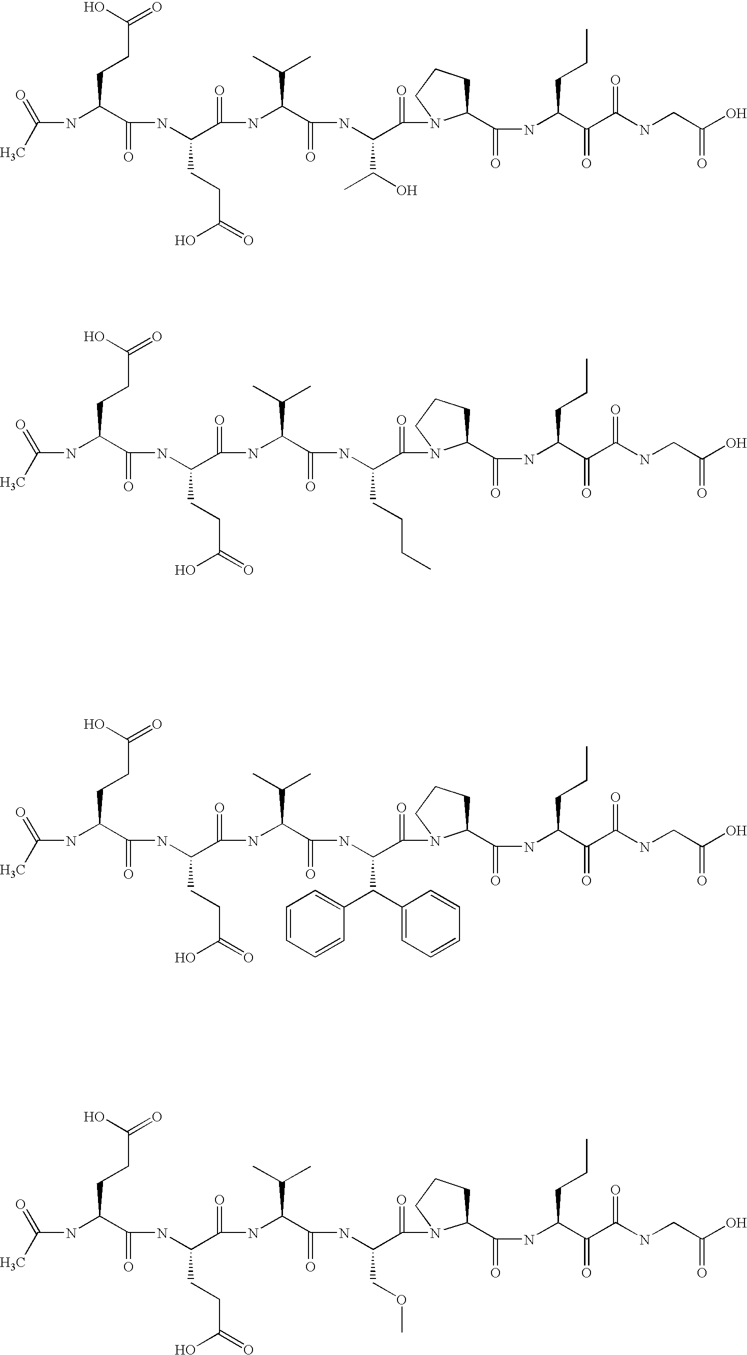 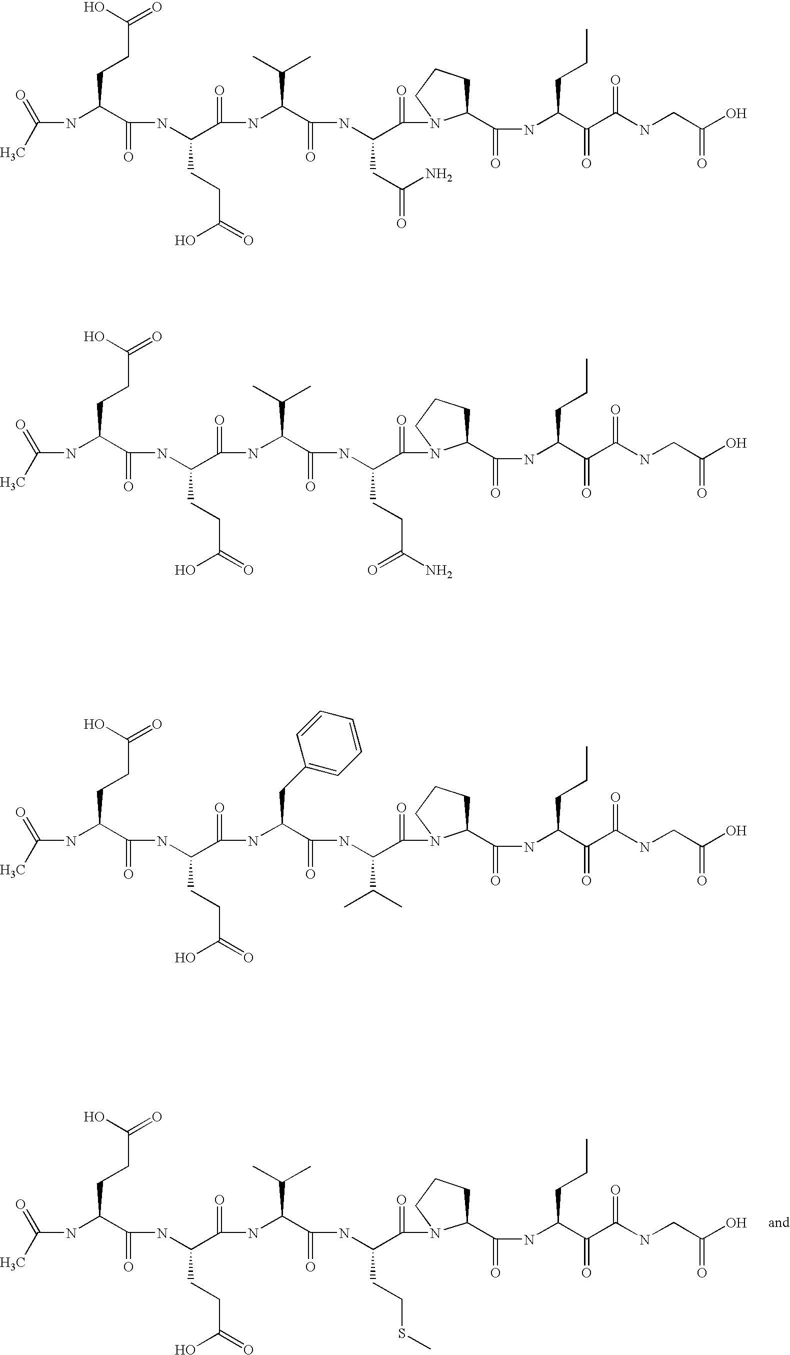 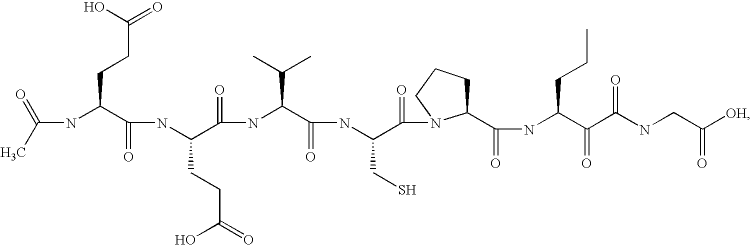
or a pharmaceutically acceptable salt, solvate or ester thereof.

The compounds of formula IIII are disclosed in International Patent Publicaton WO02/08187 published Jan. 31, 2002, and in U.S. Patent Application Publication US 2002/0160962 published Oct. 31, 2002, both of which are incorporated herein by reference.

Non-limiting examples of compounds disclosed in these publications are: 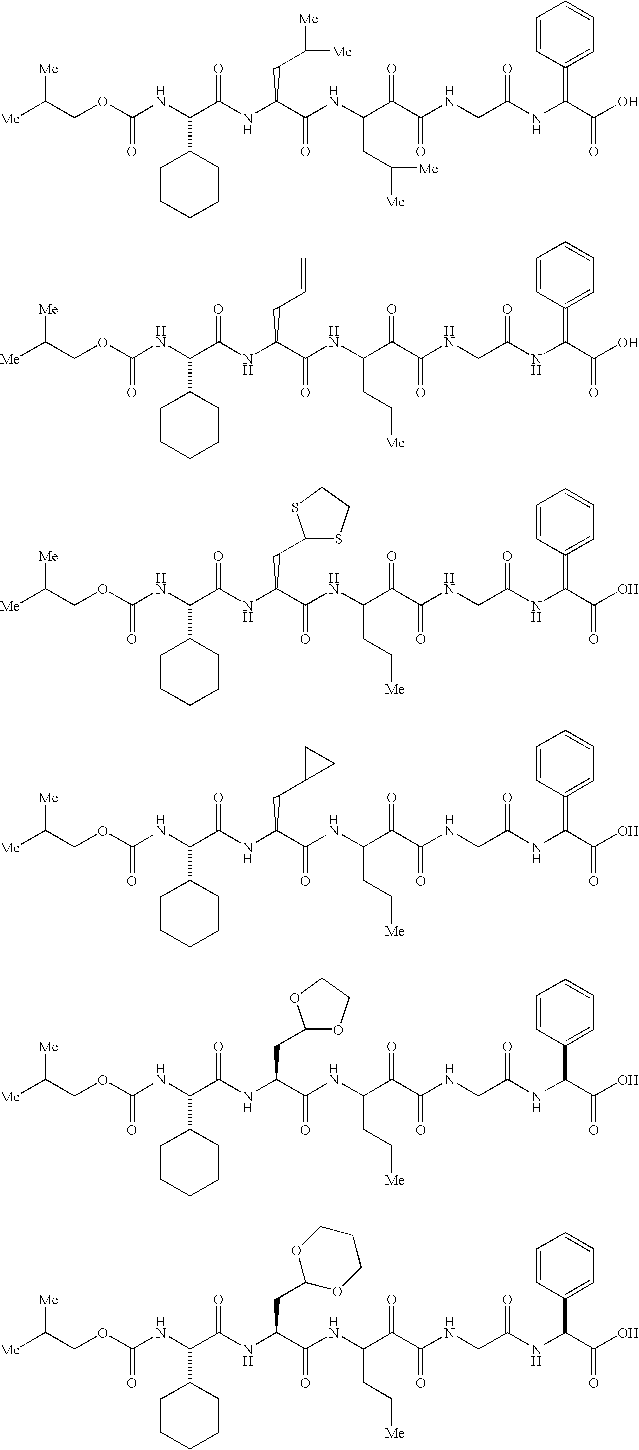 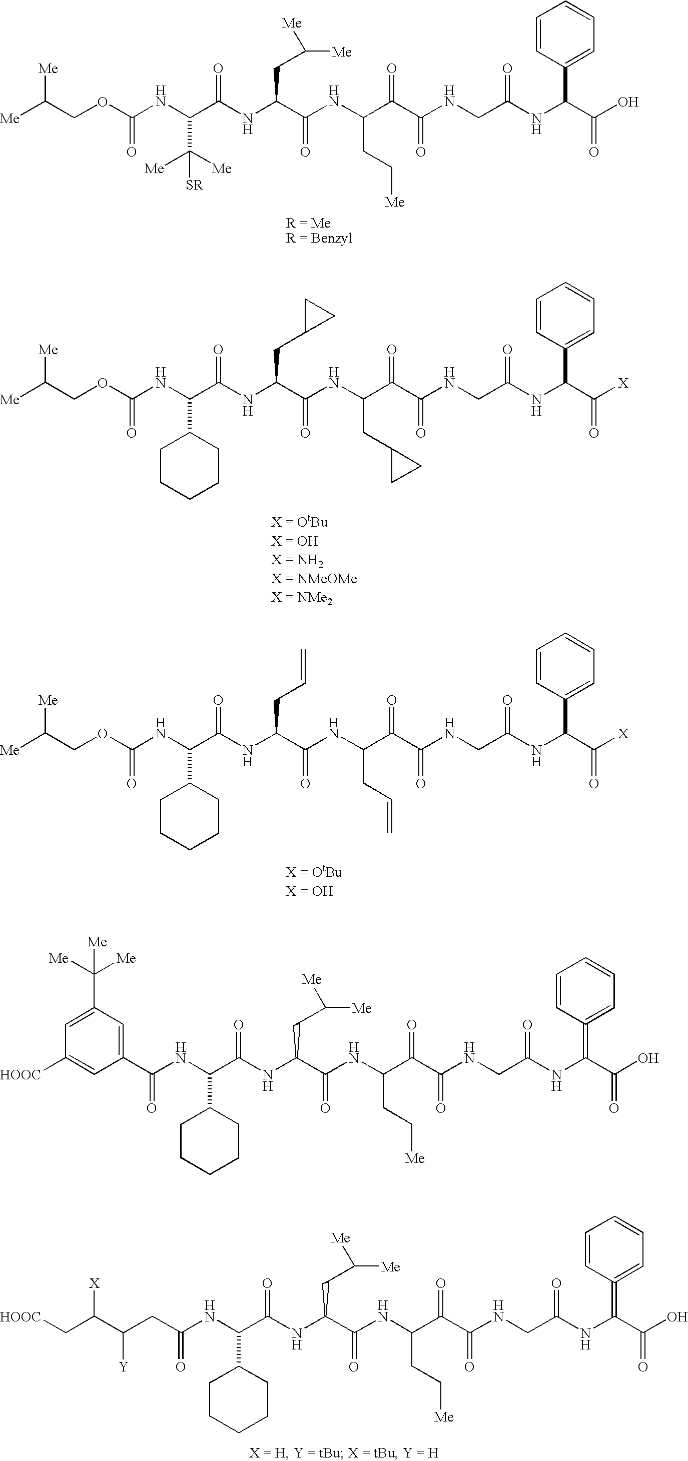 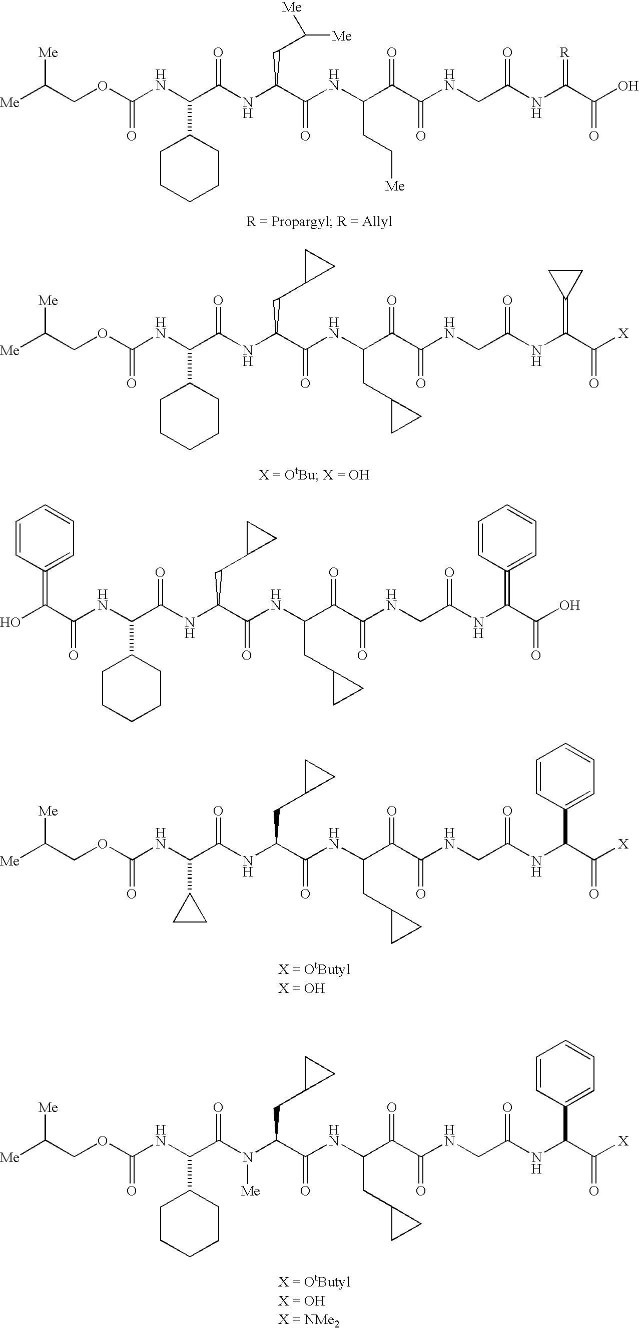 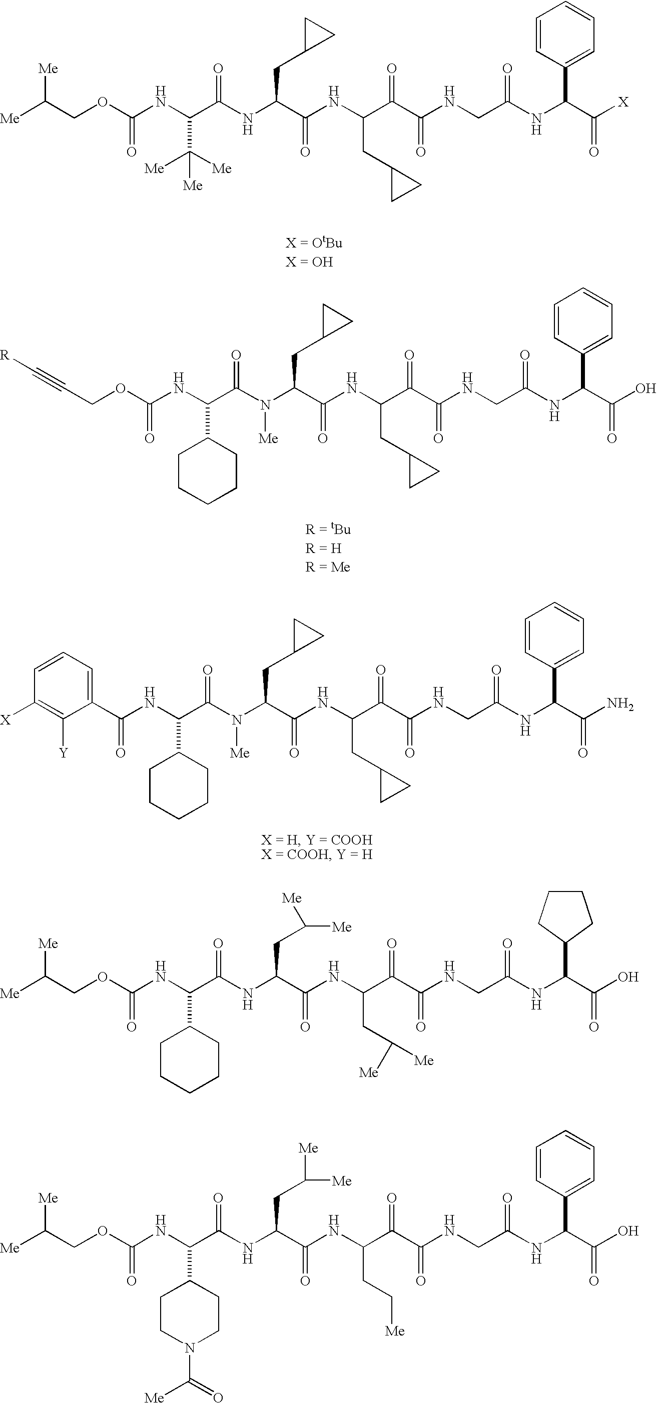 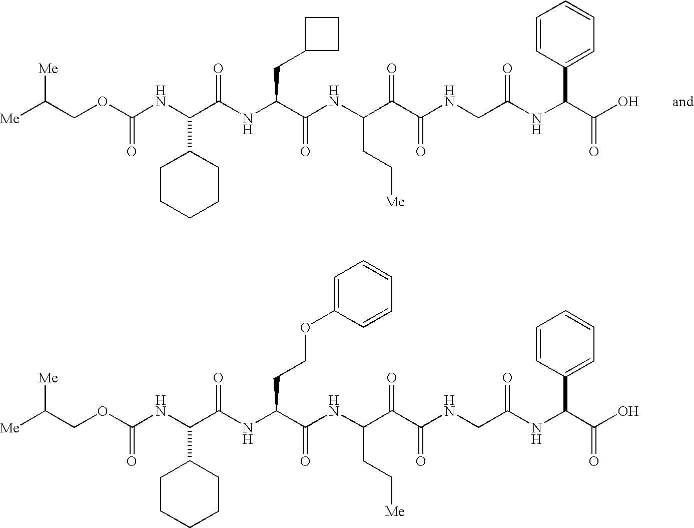
or a pharmaceutically acceptable salt, solvate or ester thereof. Other non-limiting examples are: 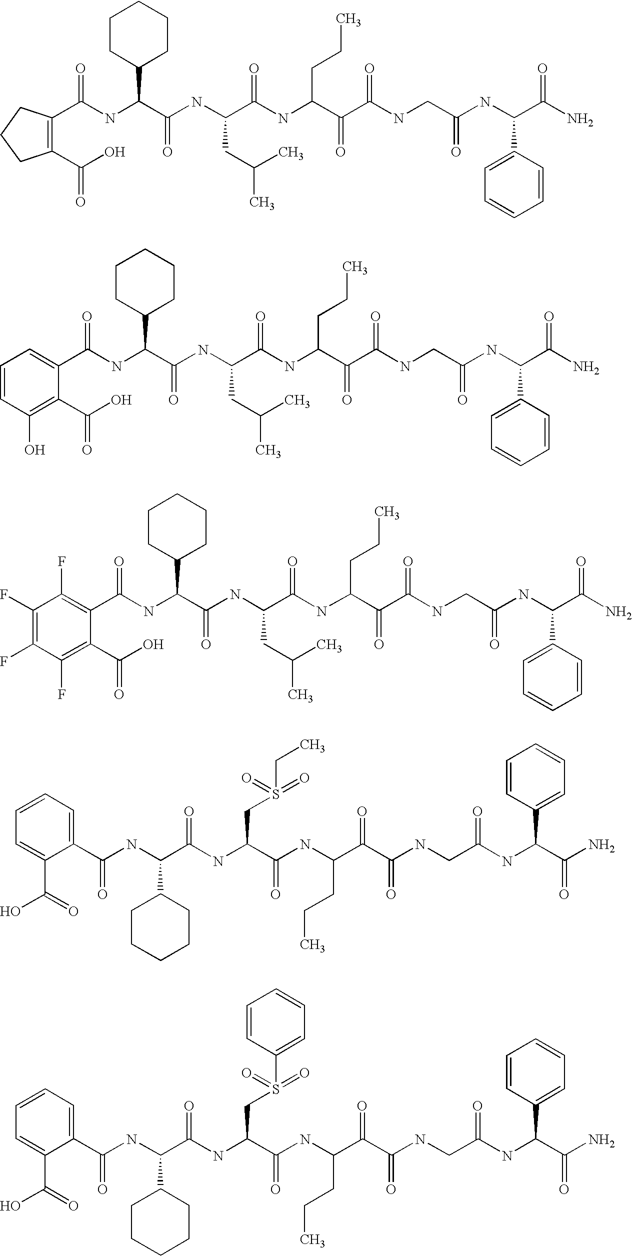 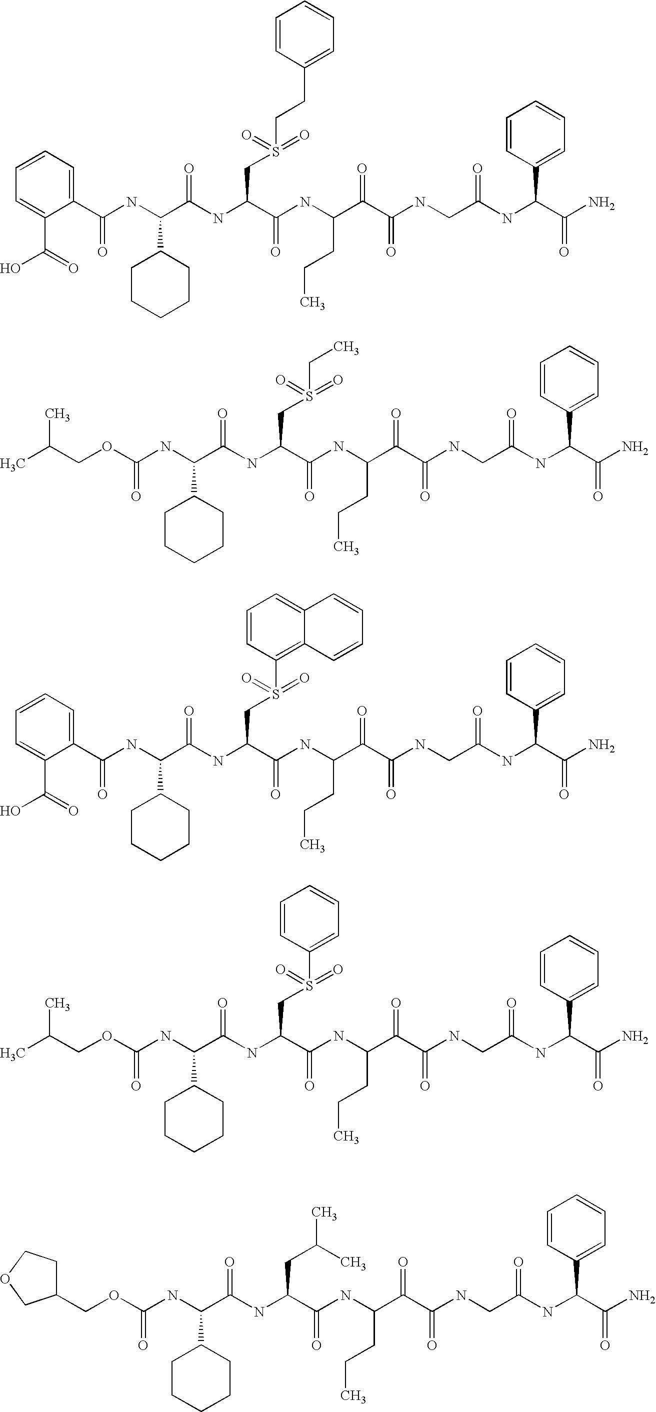 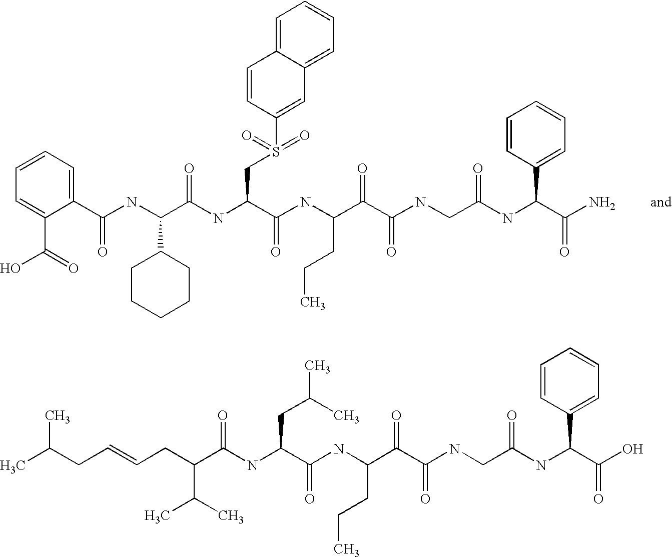
or a pharmaceutically acceptable salt or solvate thereof.

The compounds of formula IV are disclosed in International Patent Publication WO03/062228 published Jul. 31, 2003, and in U.S. Patent Application Publication 2003/0207861 published Nov. 6, 2003, both of which are incorporated herein by reference.

Non-limiting examples of compounds disclosed in these publications are: 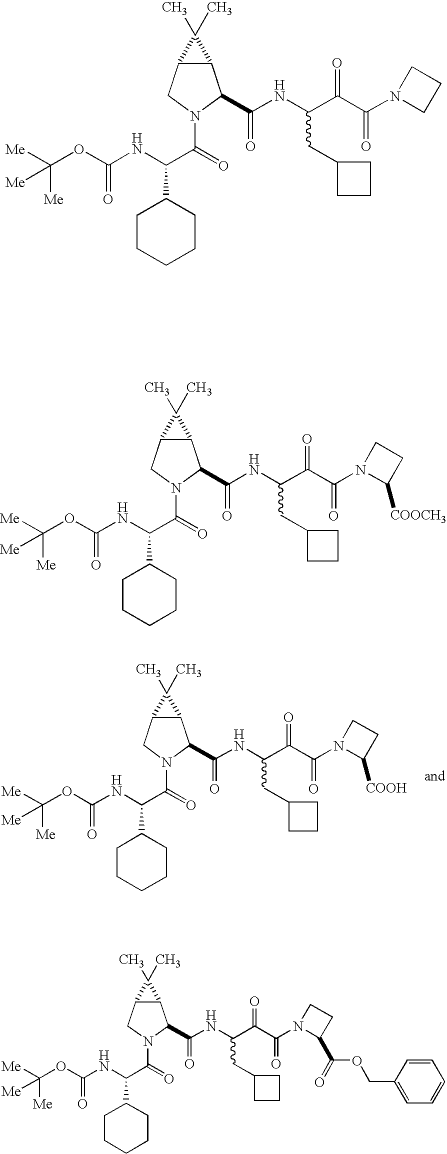
or a pharmaceutically acceptable salt or solvate of said compound.

Compounds of formula V are disclosed in U.S. Patent Application Publication US 2005/0119168 published Jun. 2, 2005, which is incorporated herein by reference in its entirety. Non-limiting examples of certain compounds disclosed in US 2005/0119168 are: 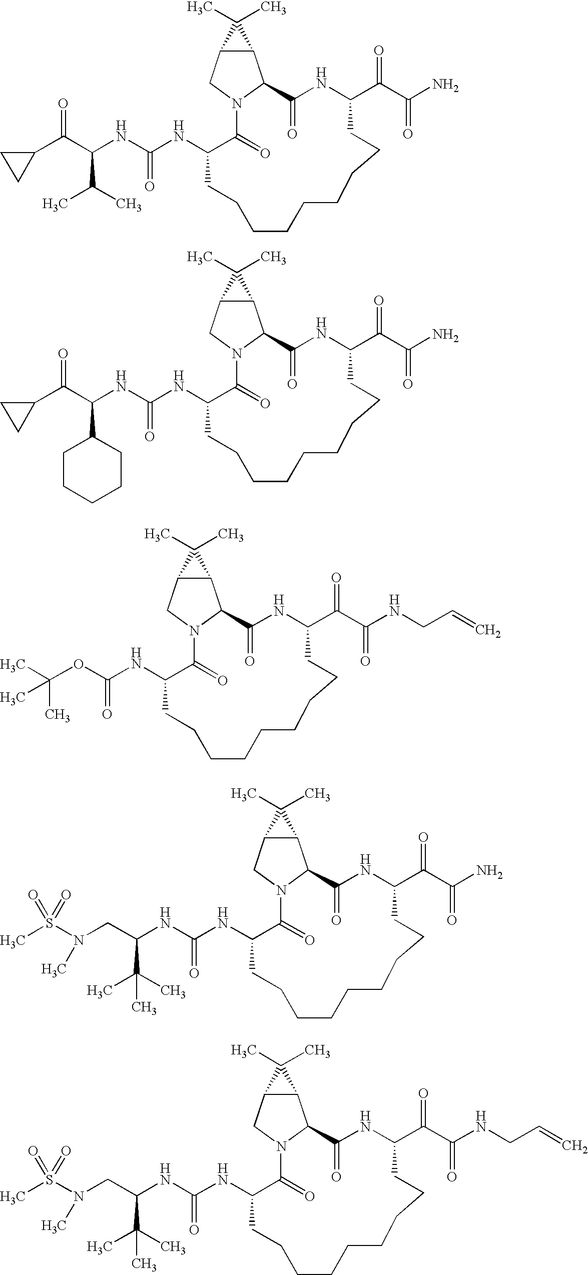 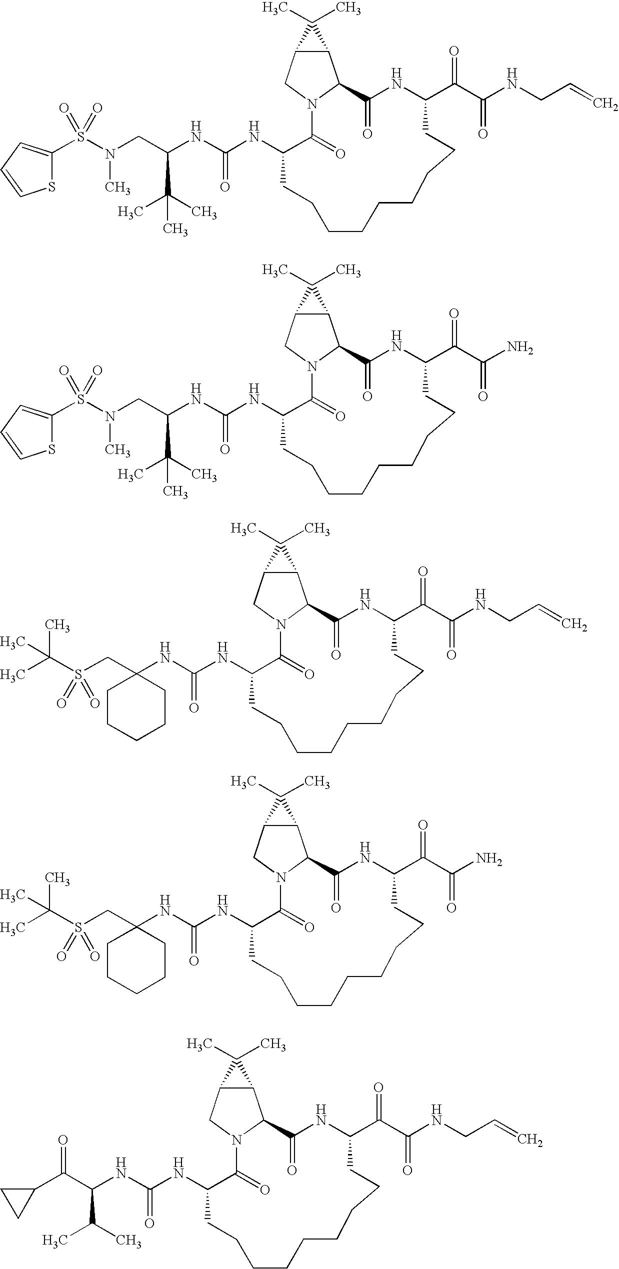 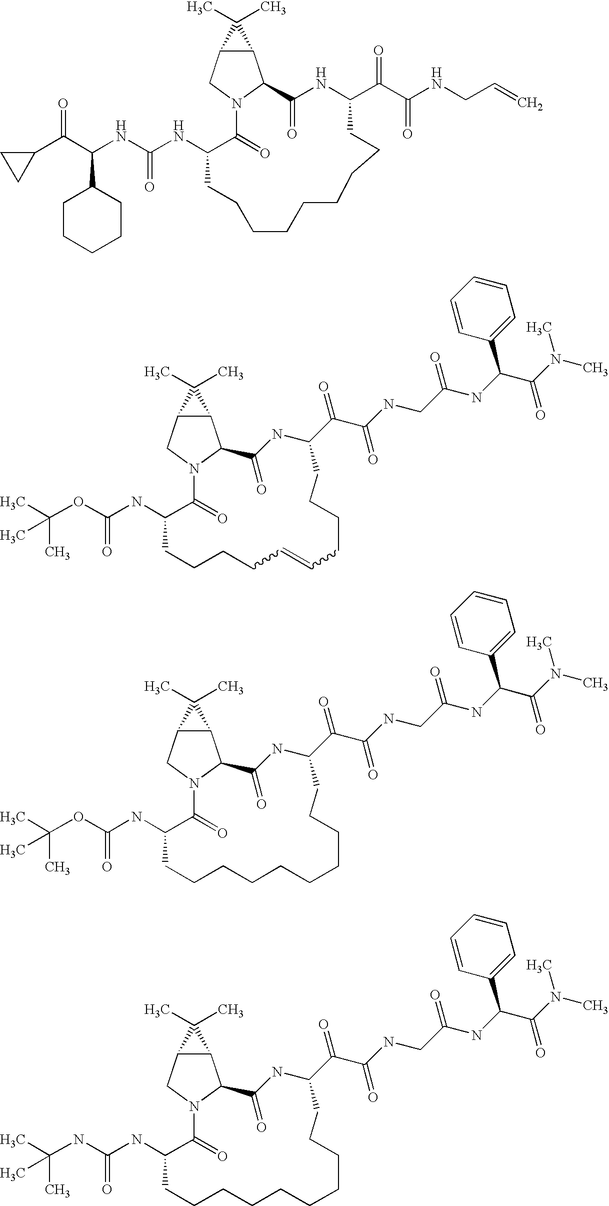 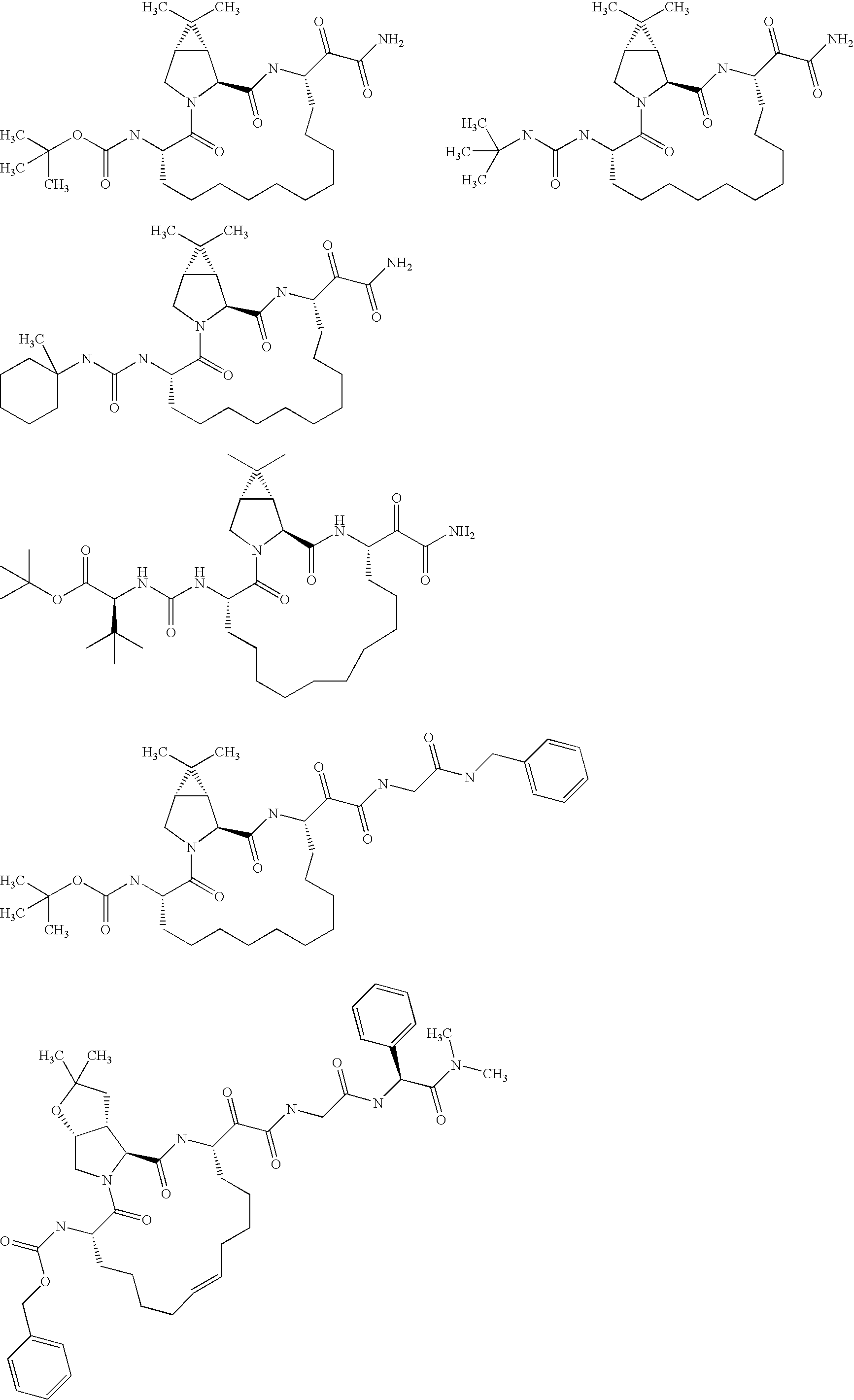 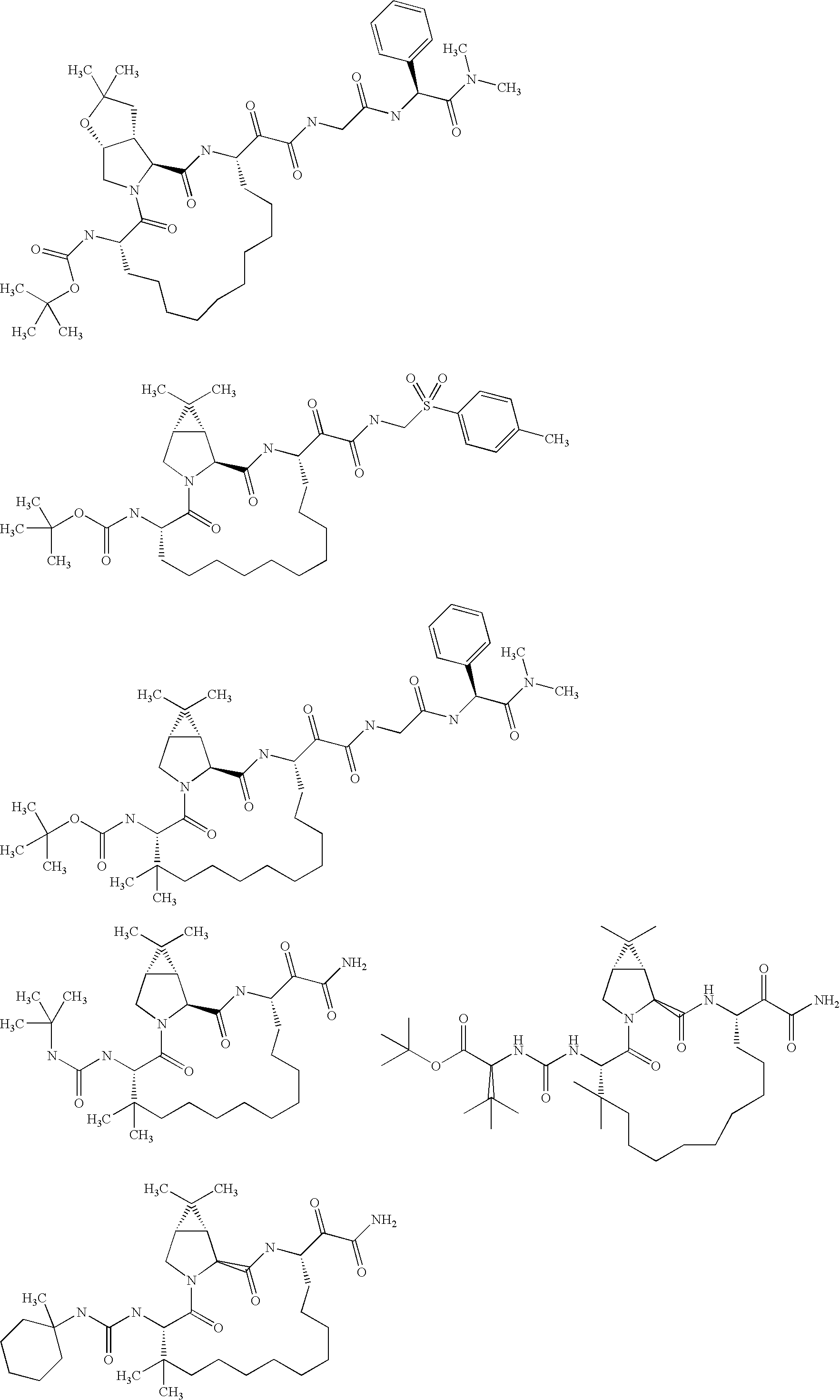
or a pharmaceutically acceptable salt, solvate or ester thereof.

Compounds of formula VI are disclosed in U.S. Patent Application Publication US 2005/0085425 published Apr. 21, 2005 and the corresponding PCT publication WO2005/021584, published Mar. 10, 2005, both of which are incoroporated herein by reference.

Compounds of formula VII-IX are disclosed in U.S. Patent Application Publication US 2005/0164921, published Jul. 28, 2005, and the corresponding PCT publication WO2005/051980, published Jun. 9, 2005, both of which are incorporated herein by reference.

Non-limiting examples of certain compounds of formula VII disclosed in these publications are: 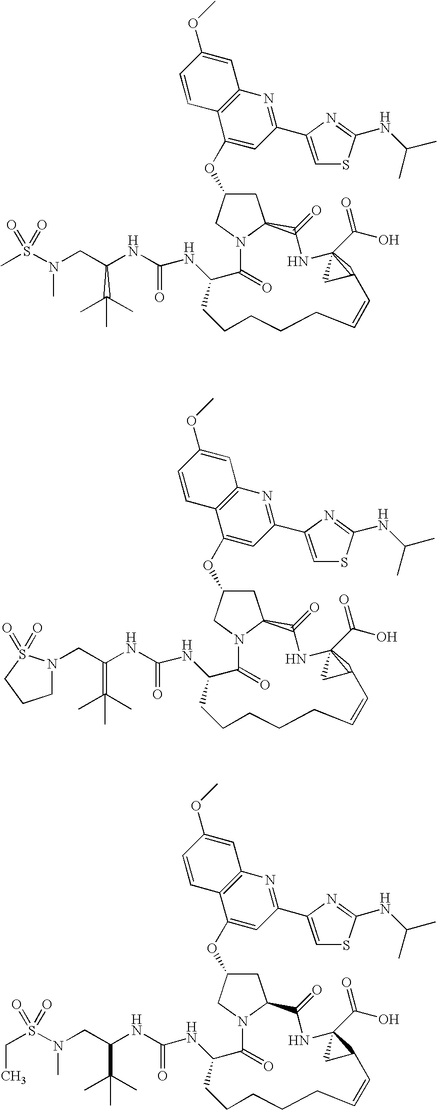 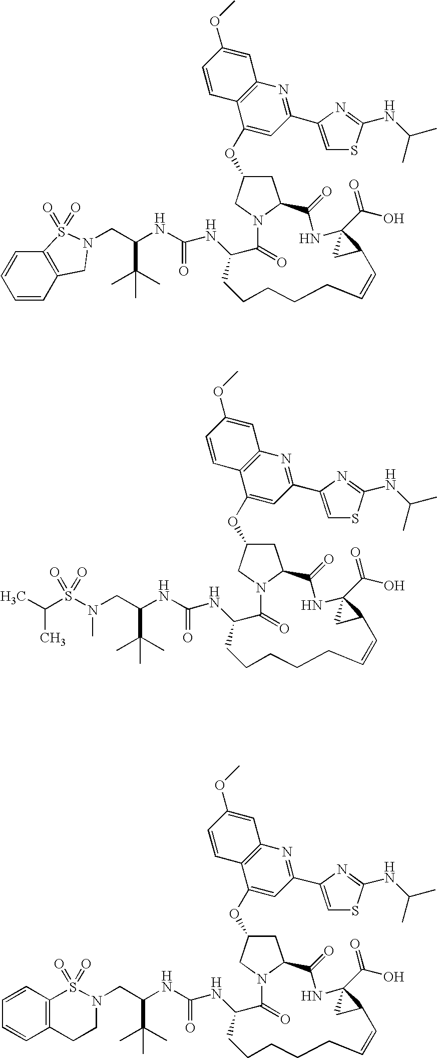 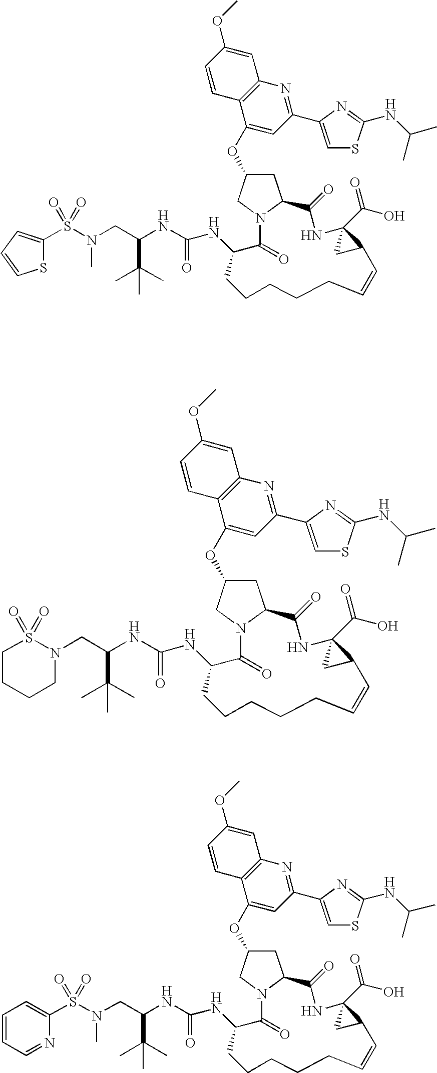 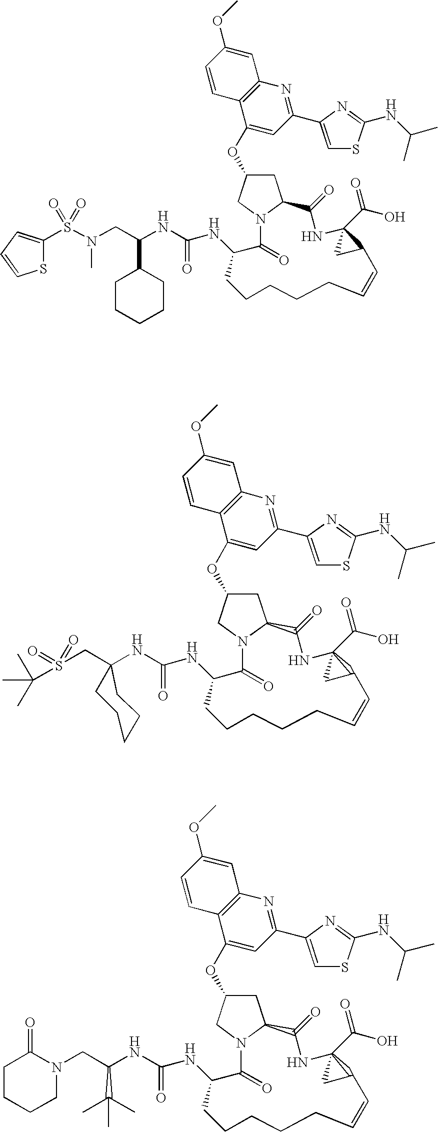 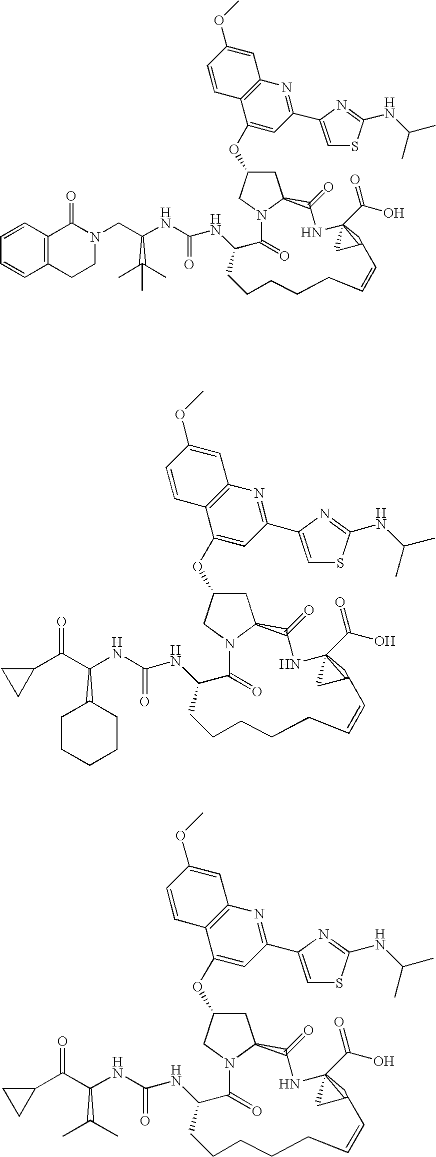 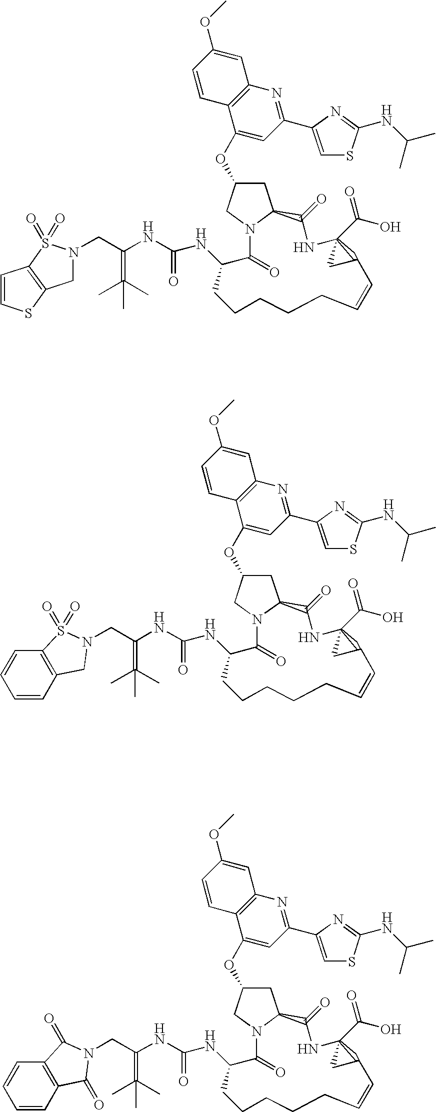 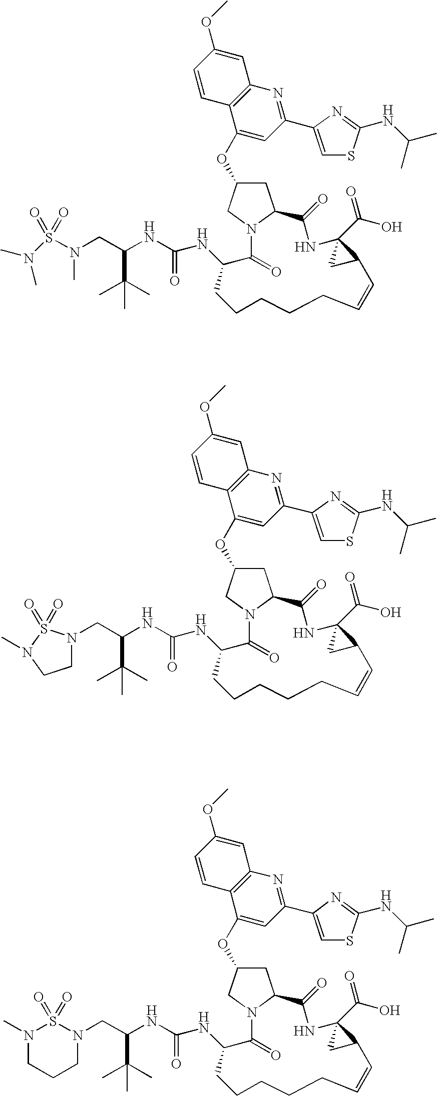 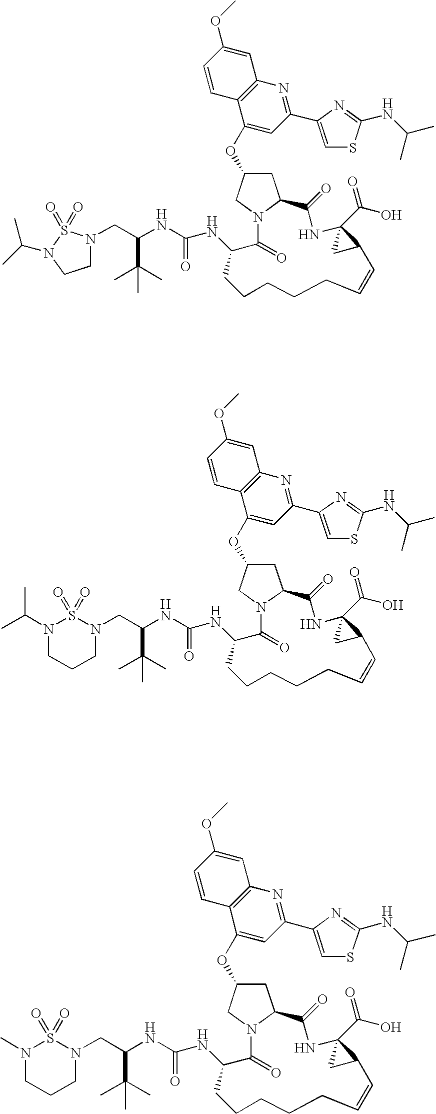 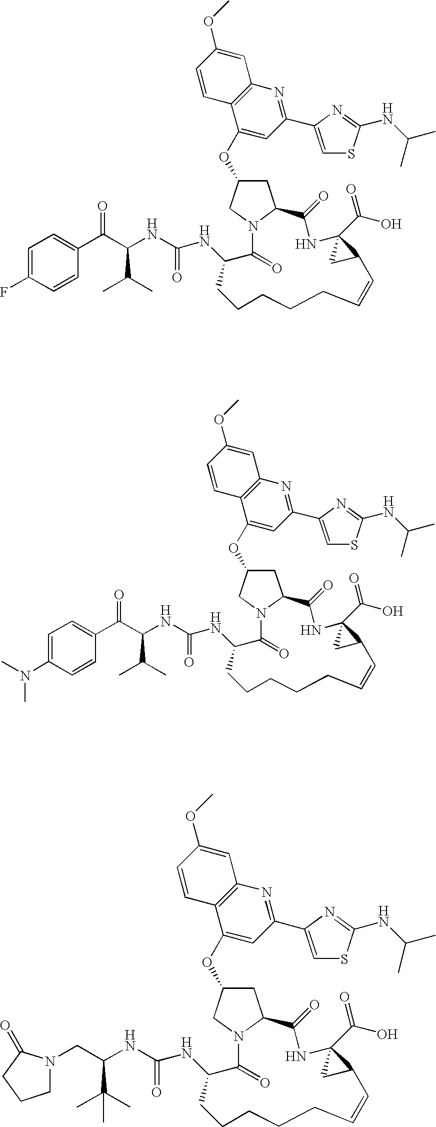 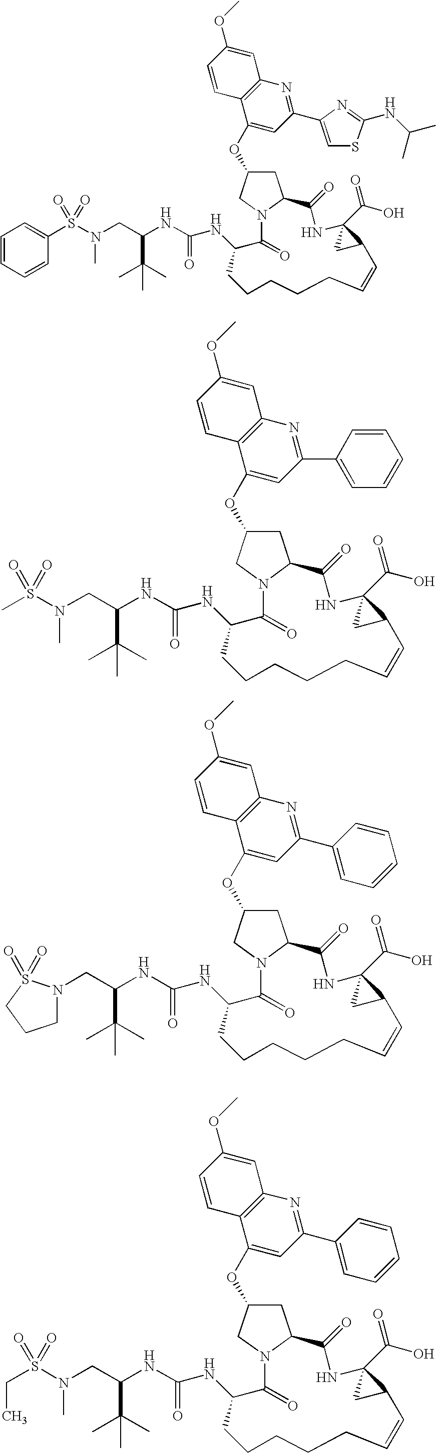 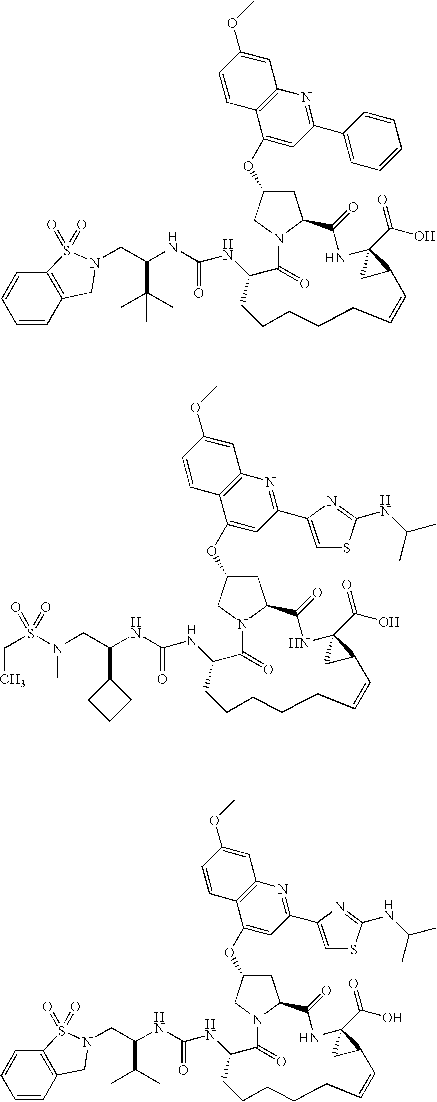 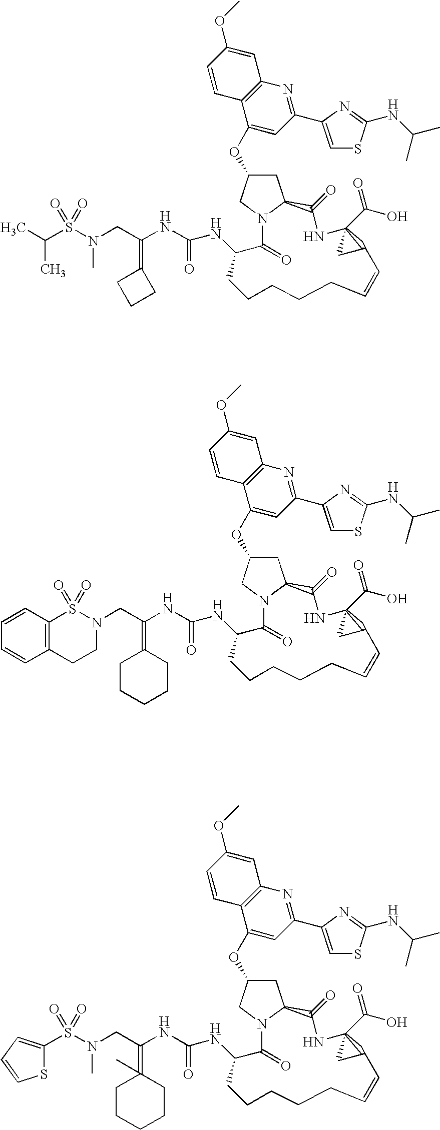 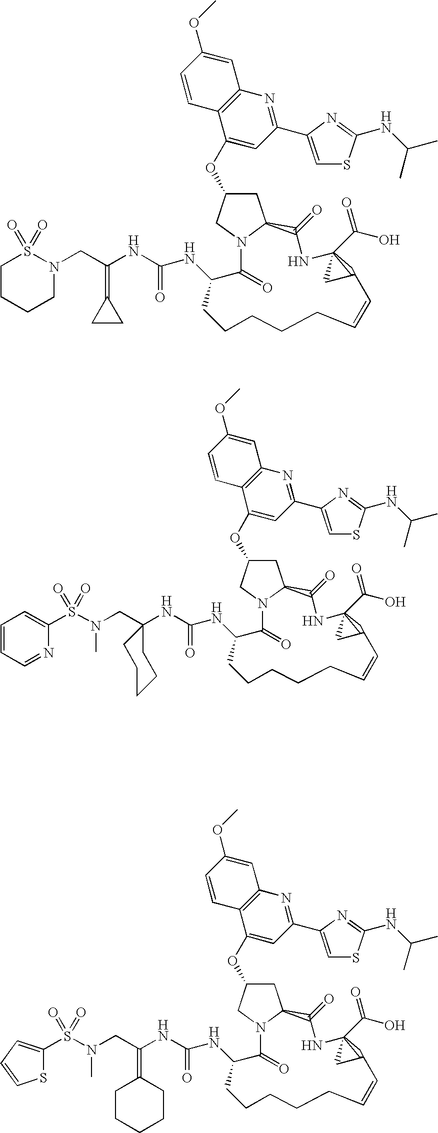 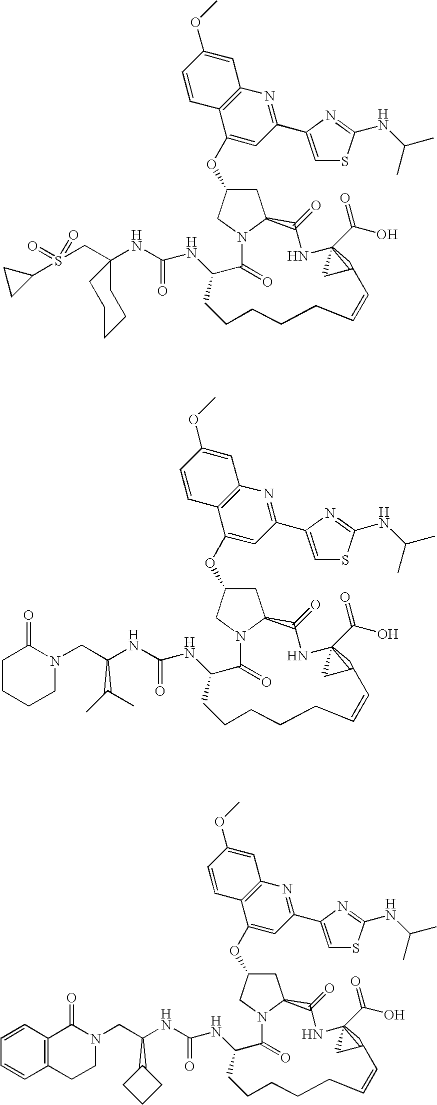 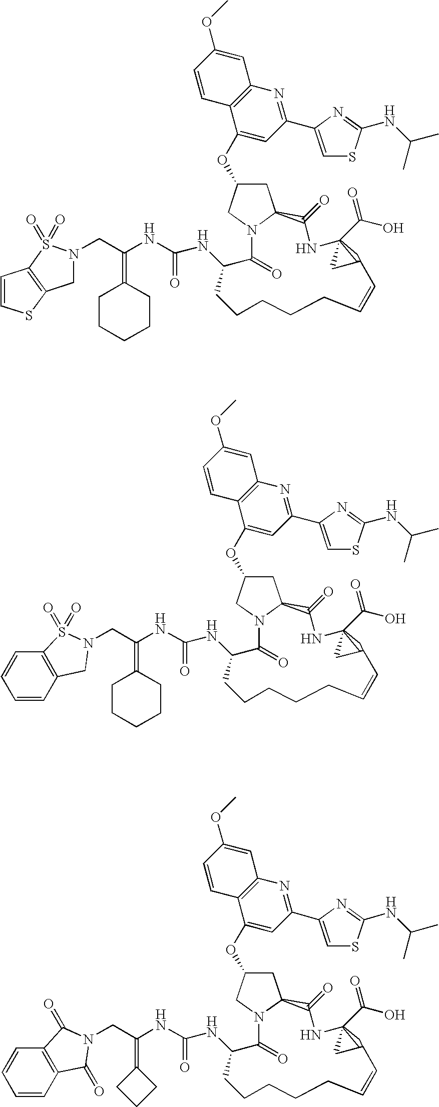 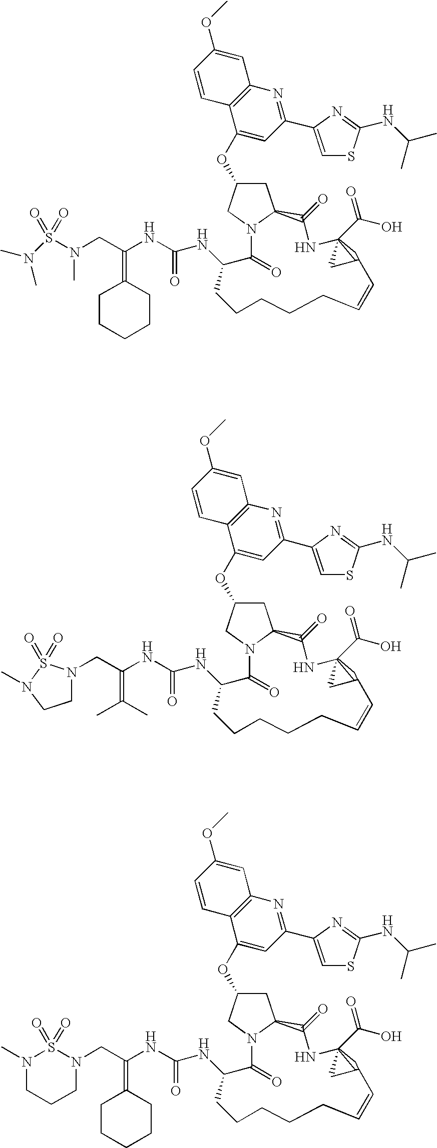 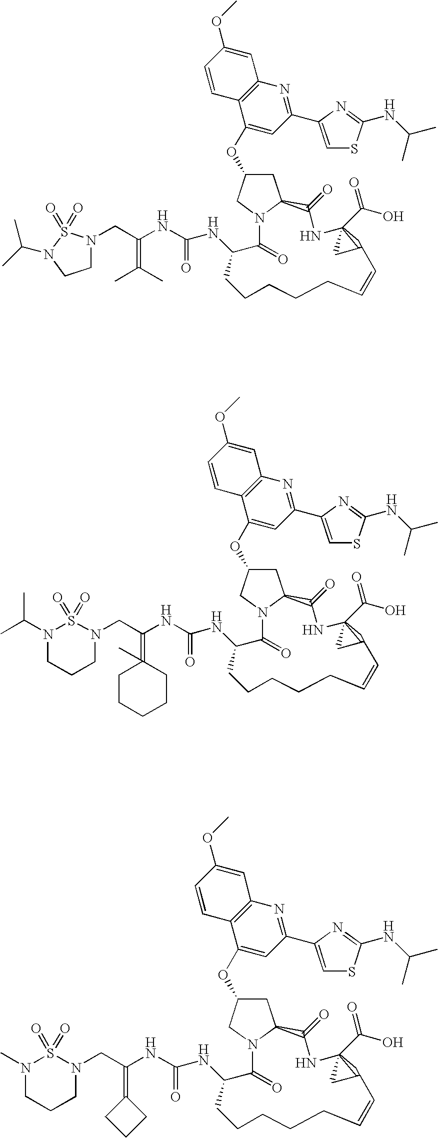 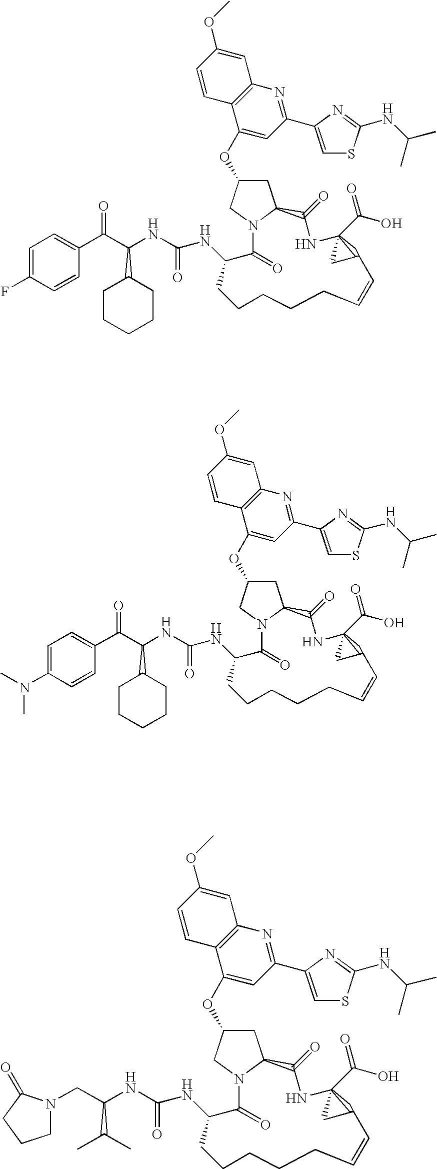 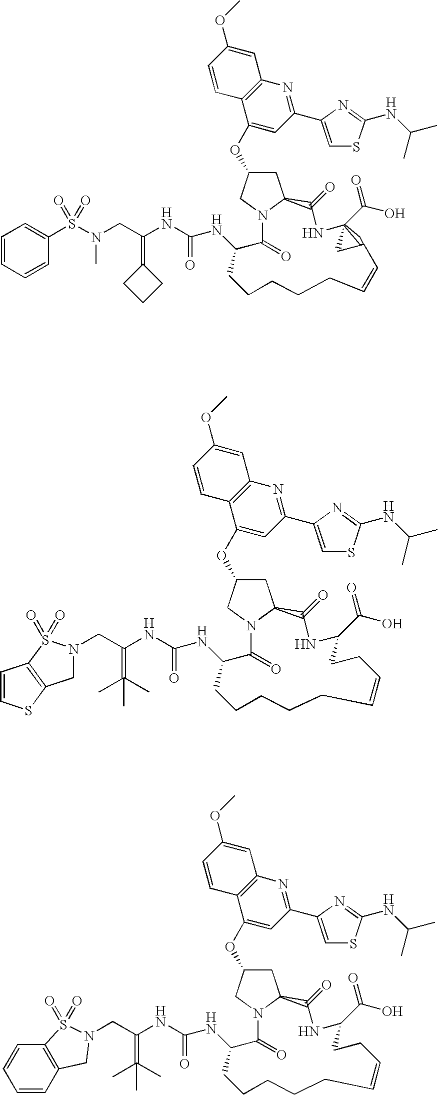 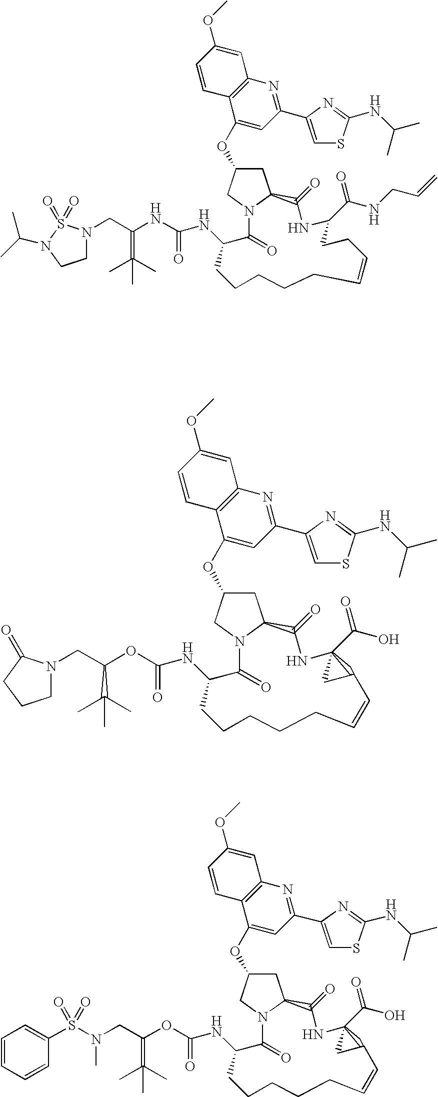 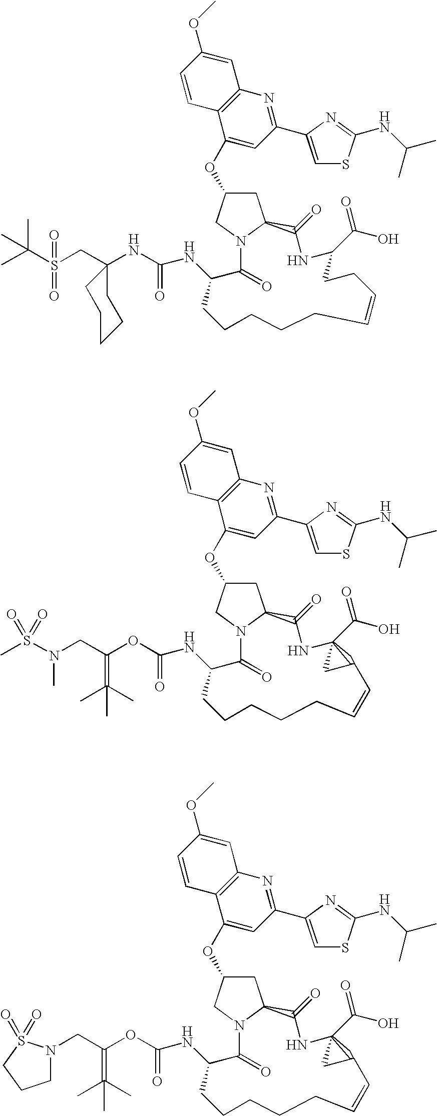 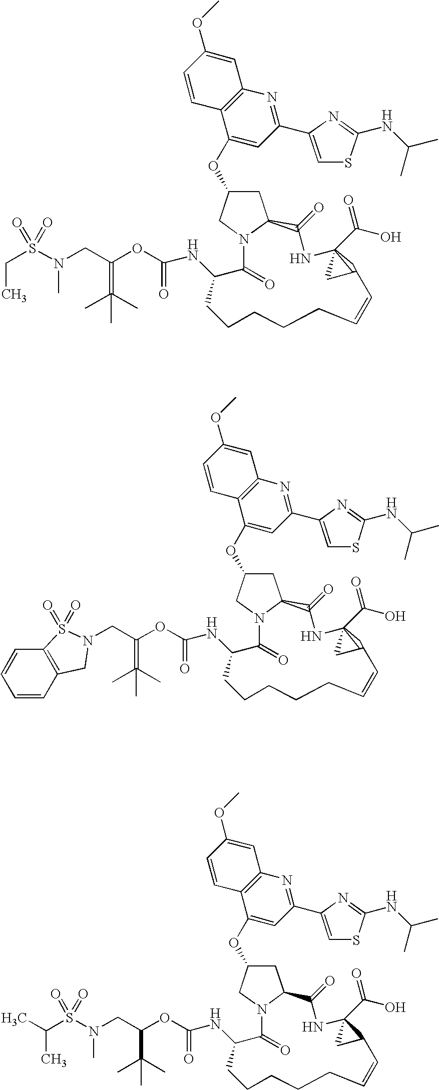 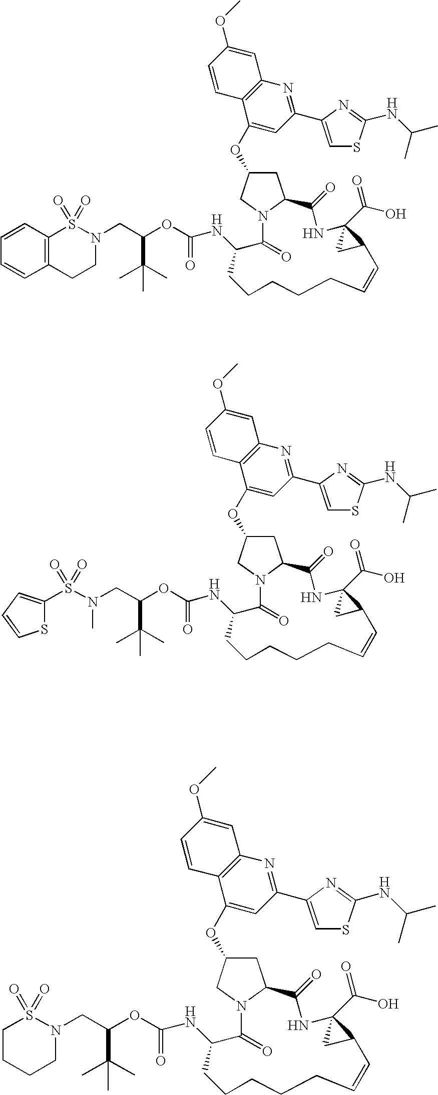 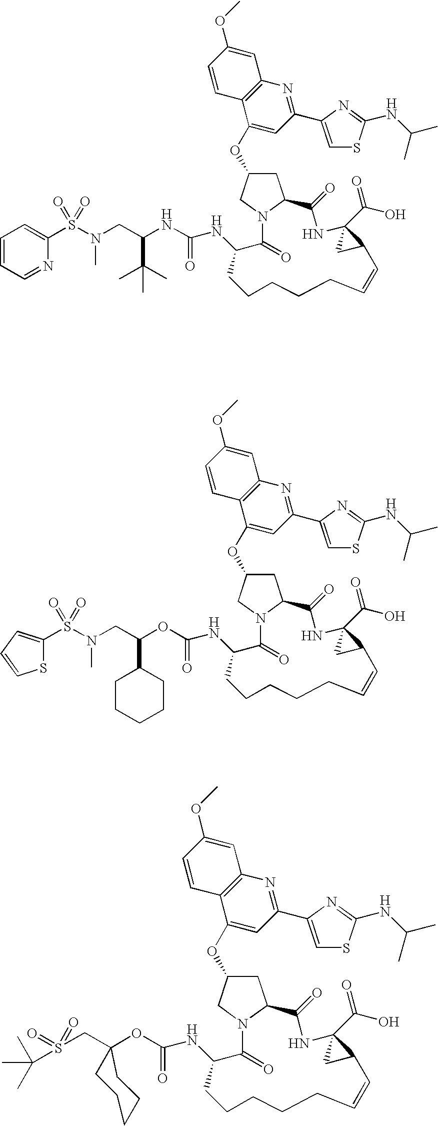 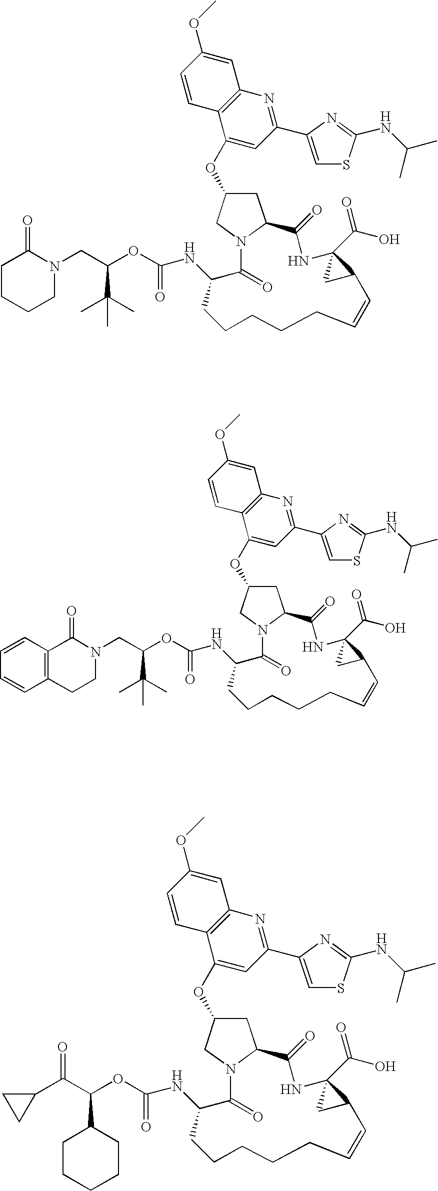 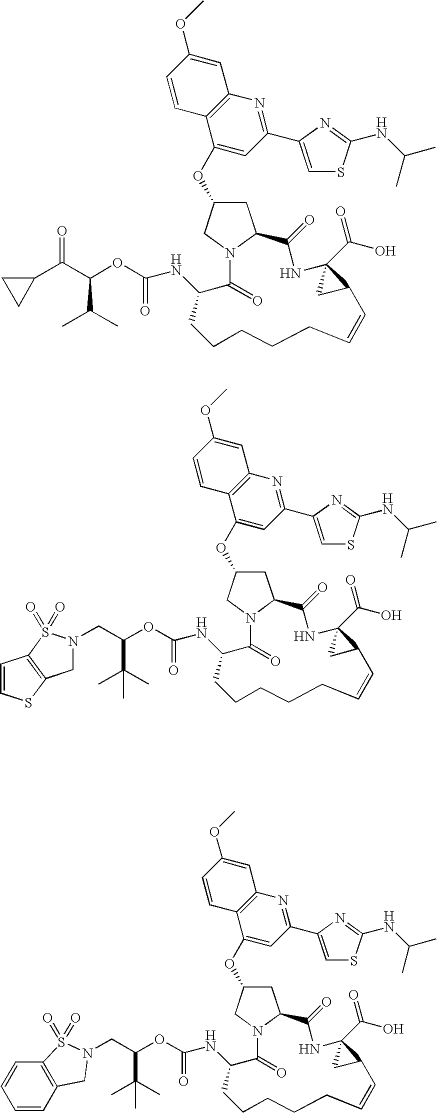 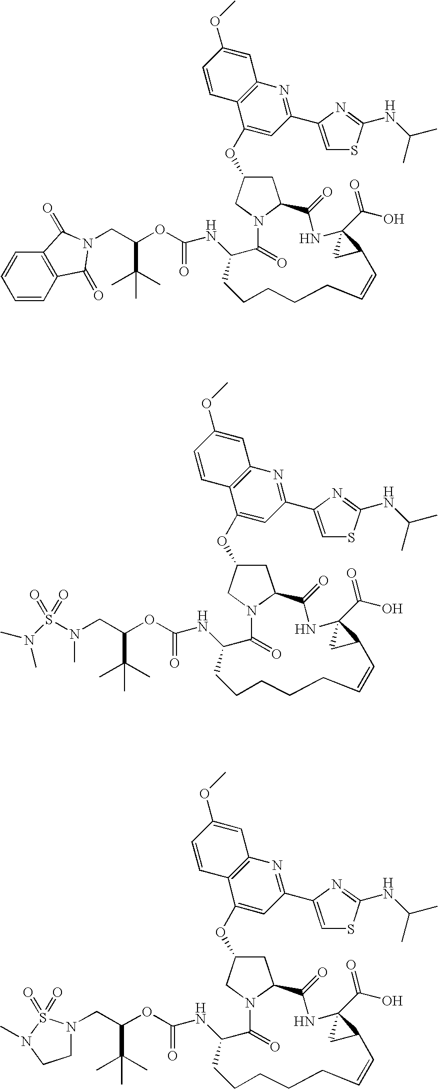 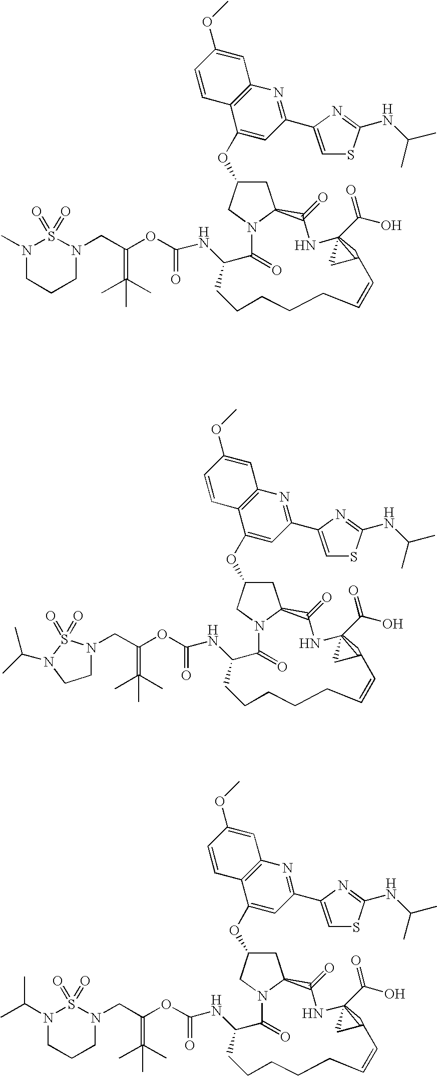 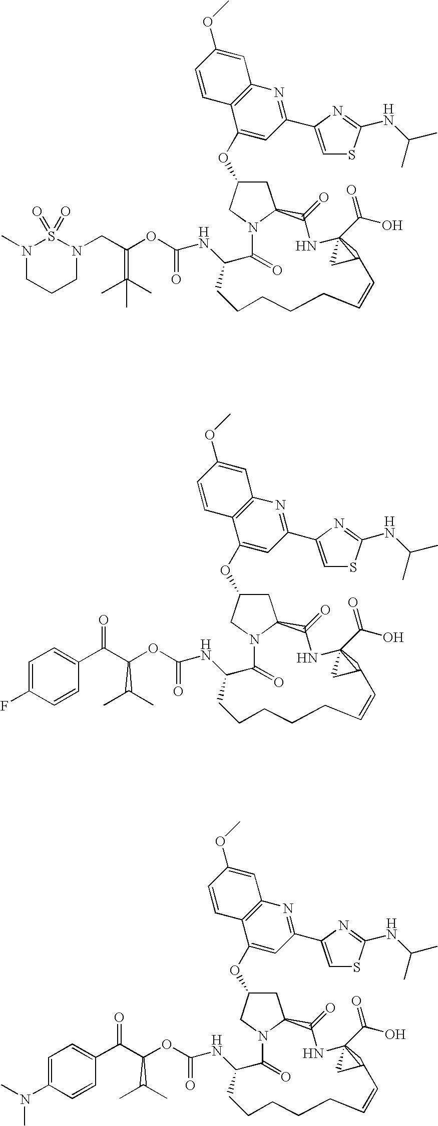 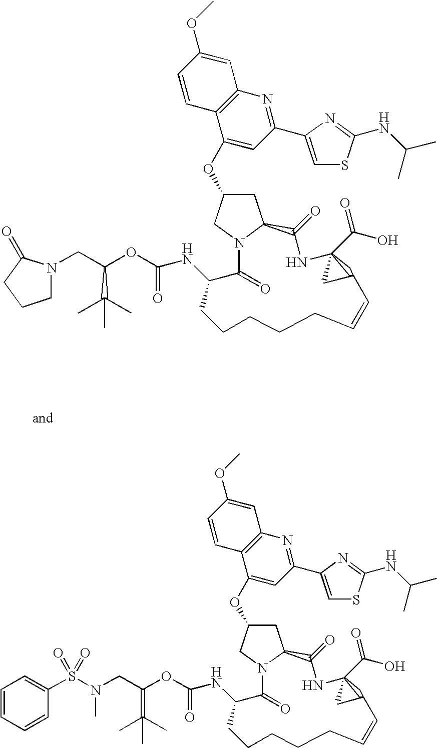
or a pharmaceutically acceptable salt, solvate or ester thereof.

Nonlimiting examples of certain compounds of formula VIII disclosed in these publications are: 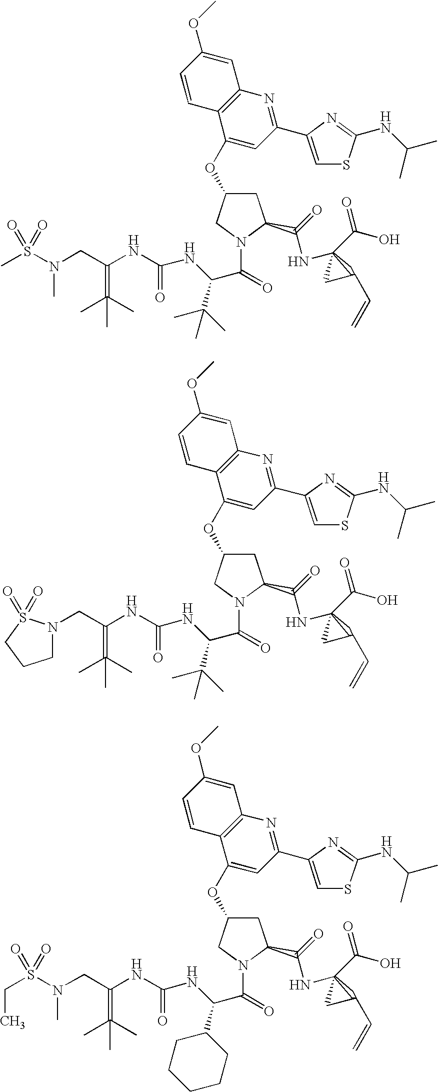 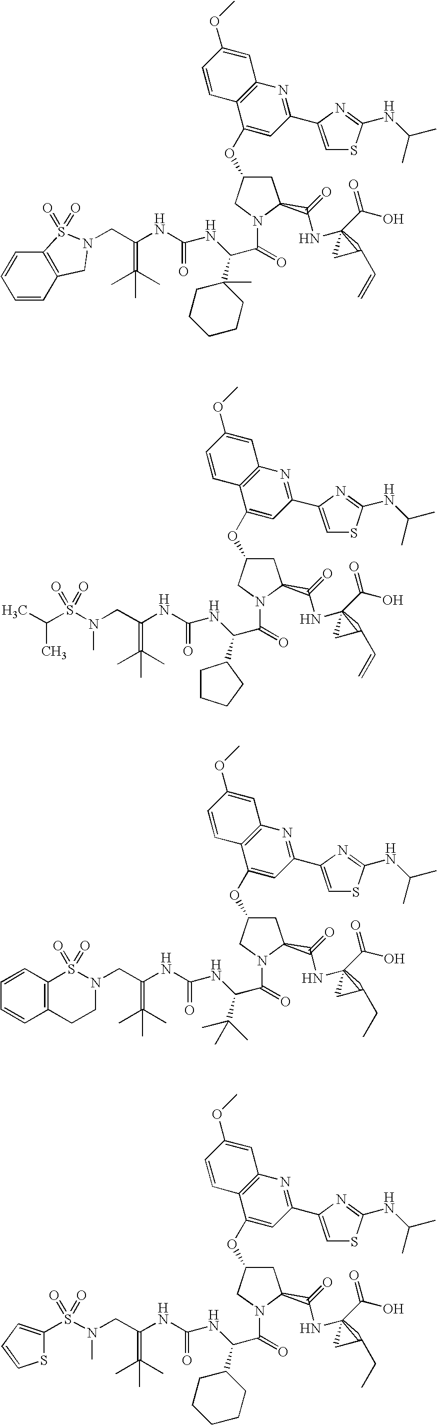 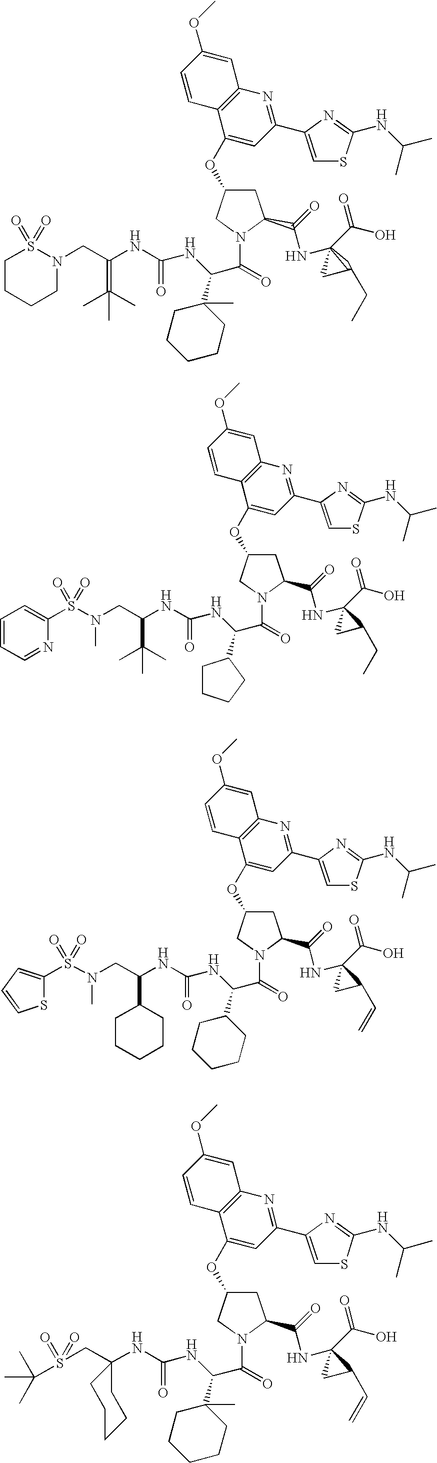 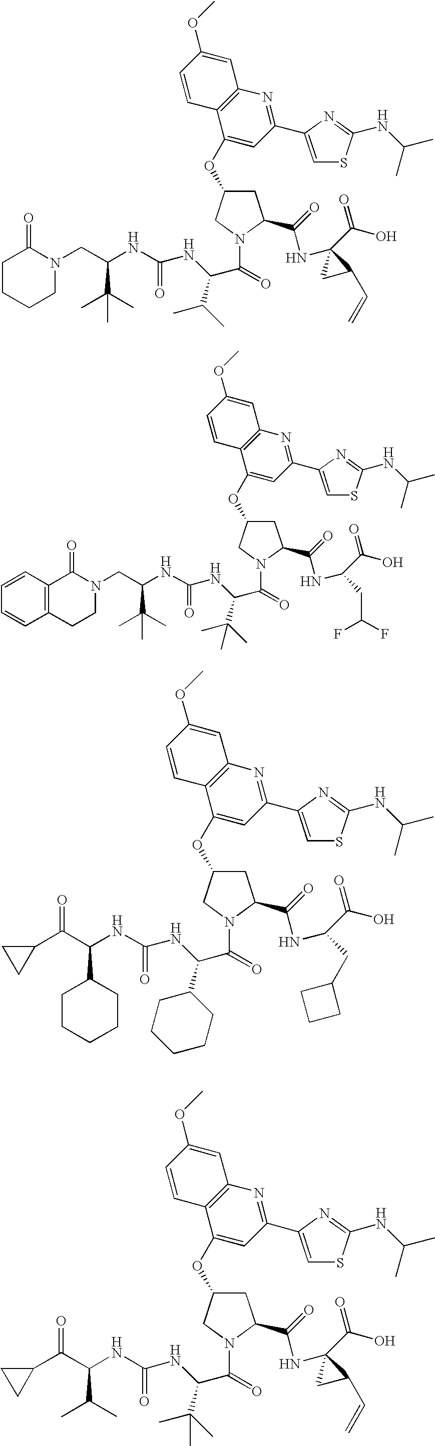 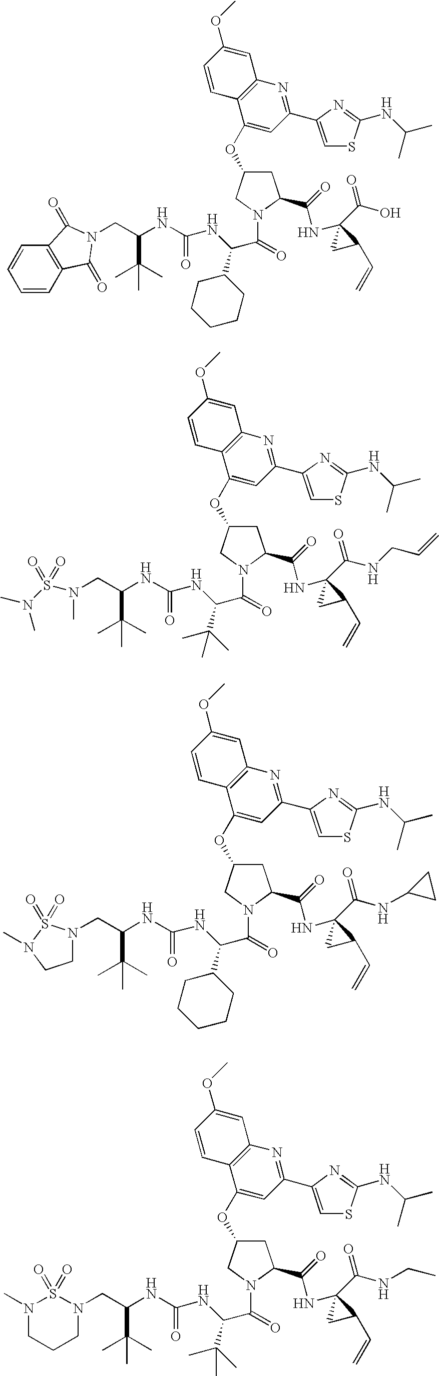 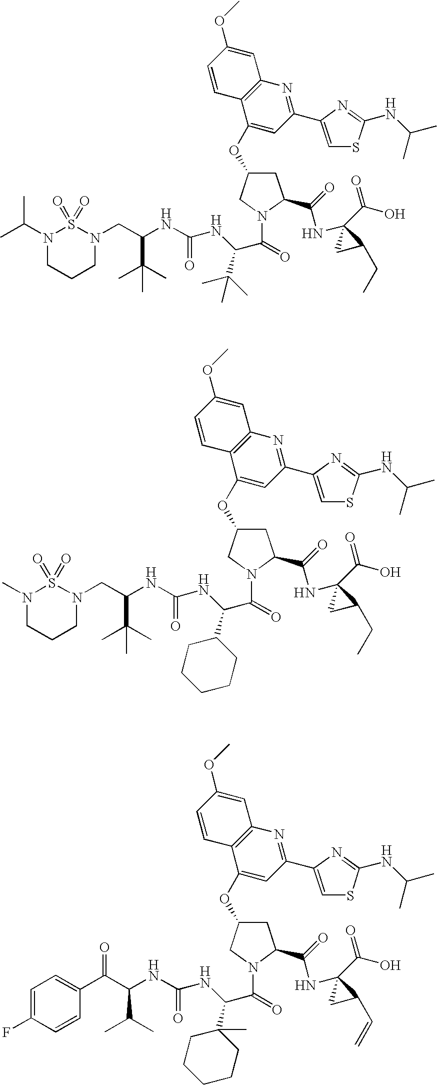 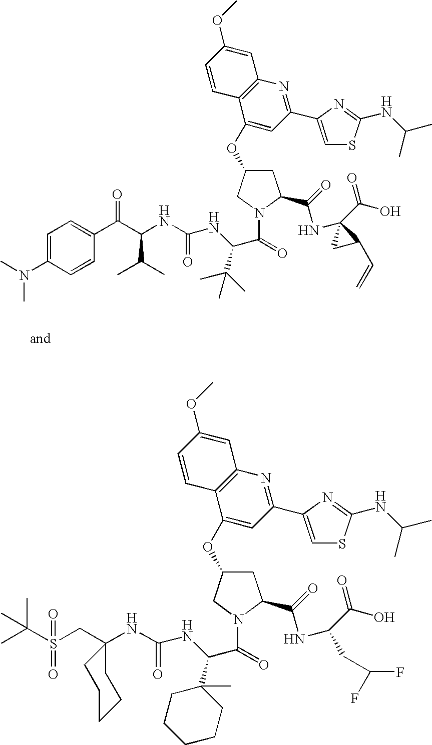
or a pharmaceutically acceptable salt, solvate or ester thereof.

Nonlimiting examples of certain compounds of formula IX disclosed in these publications are: 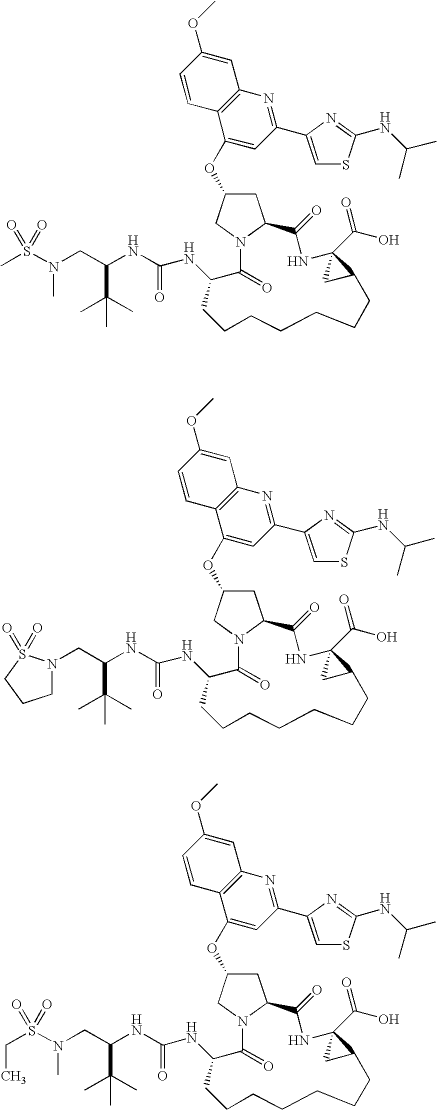 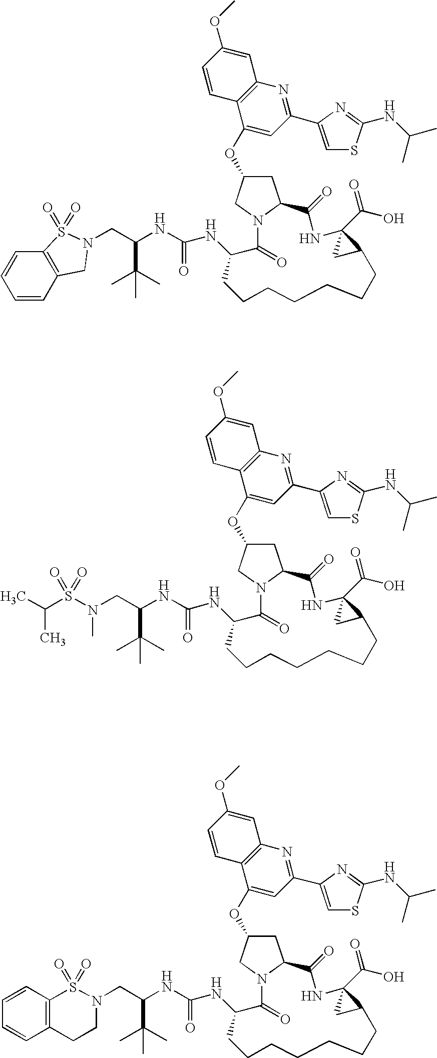 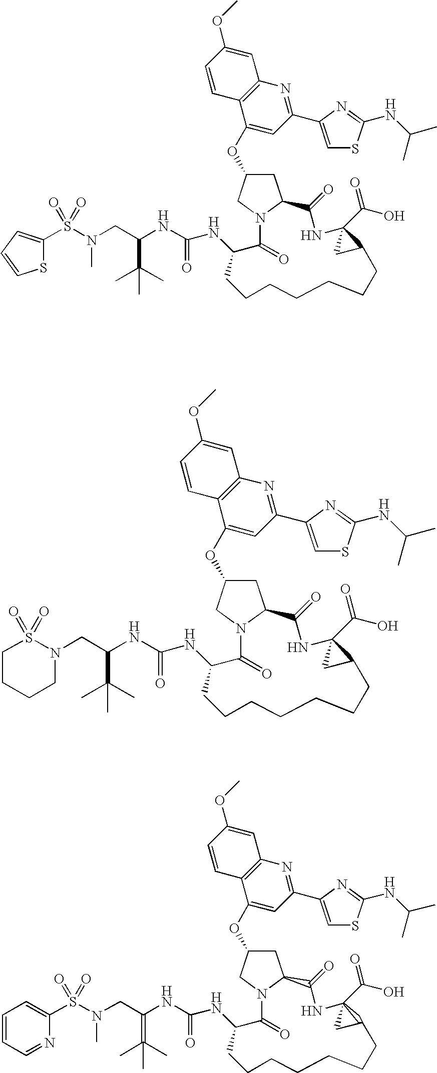 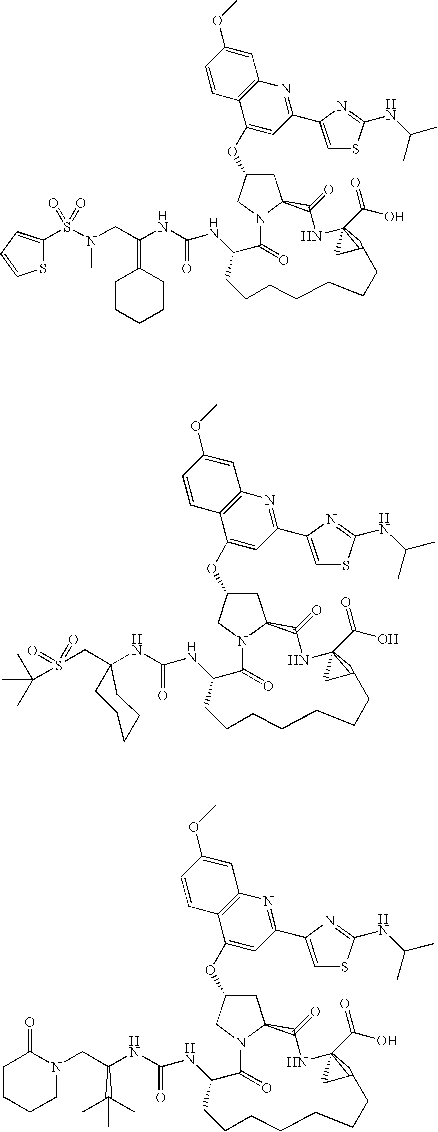 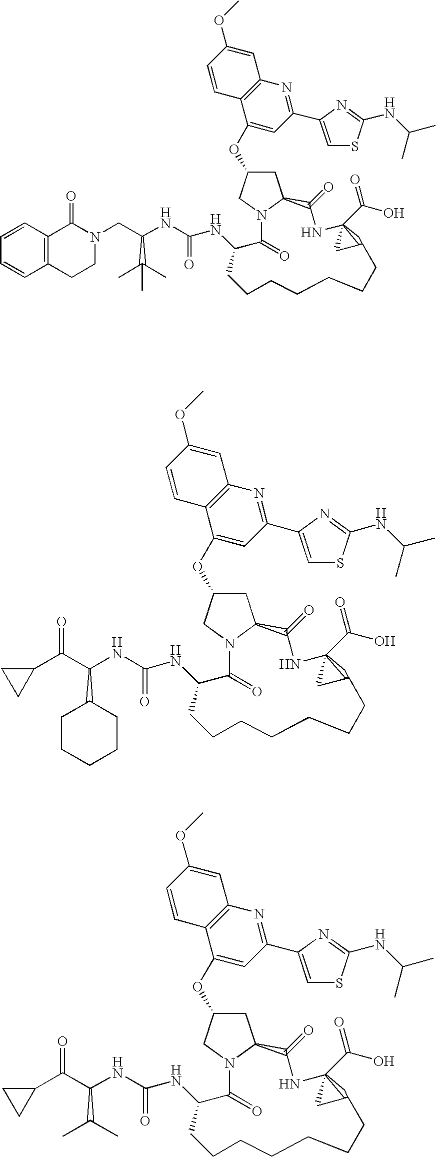 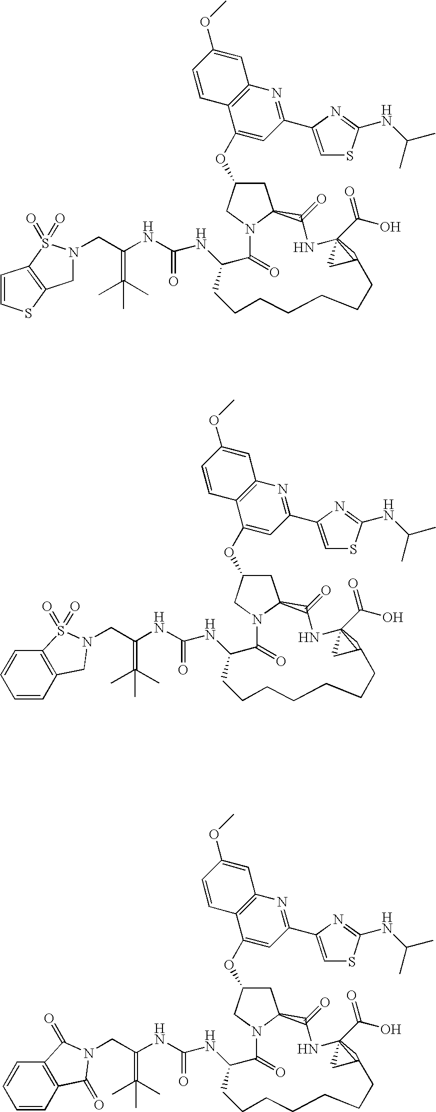 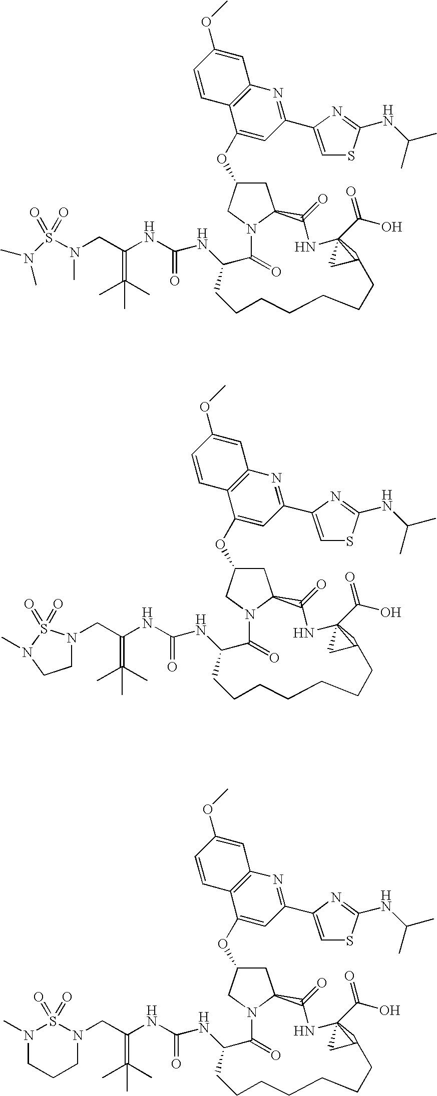 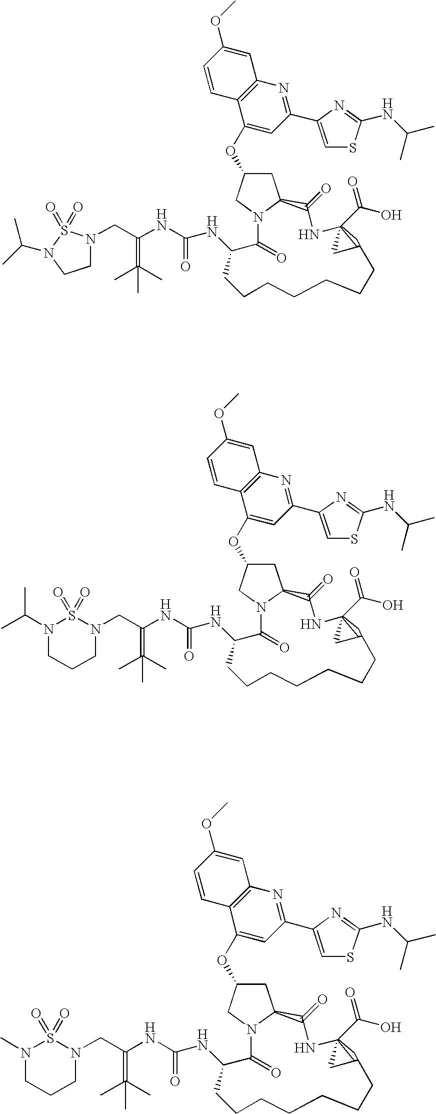 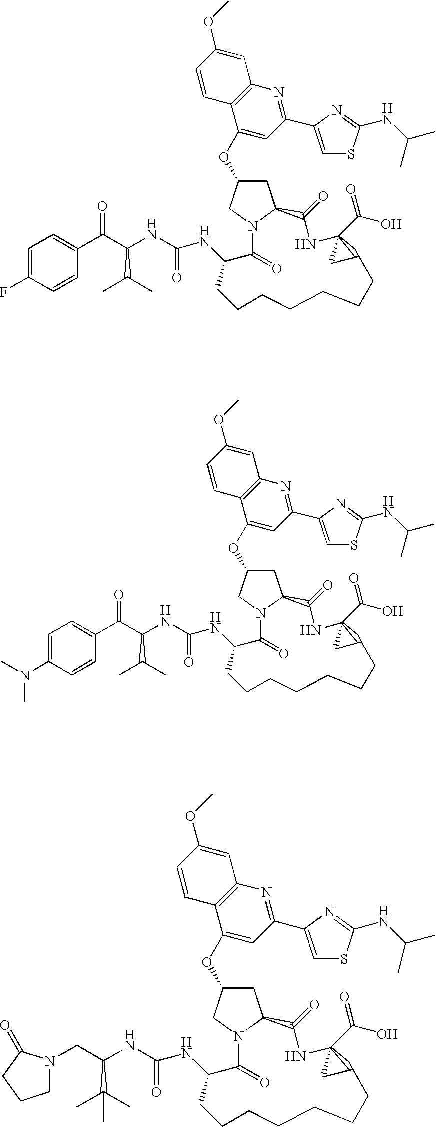 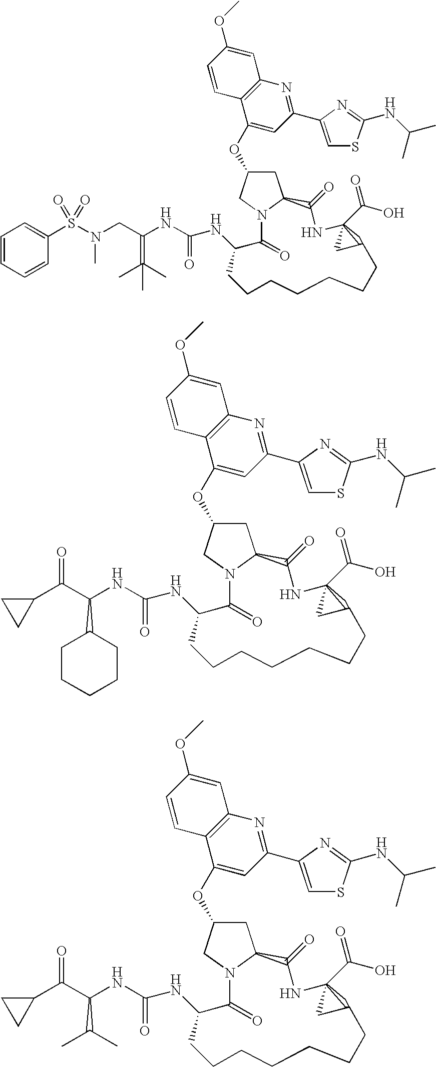 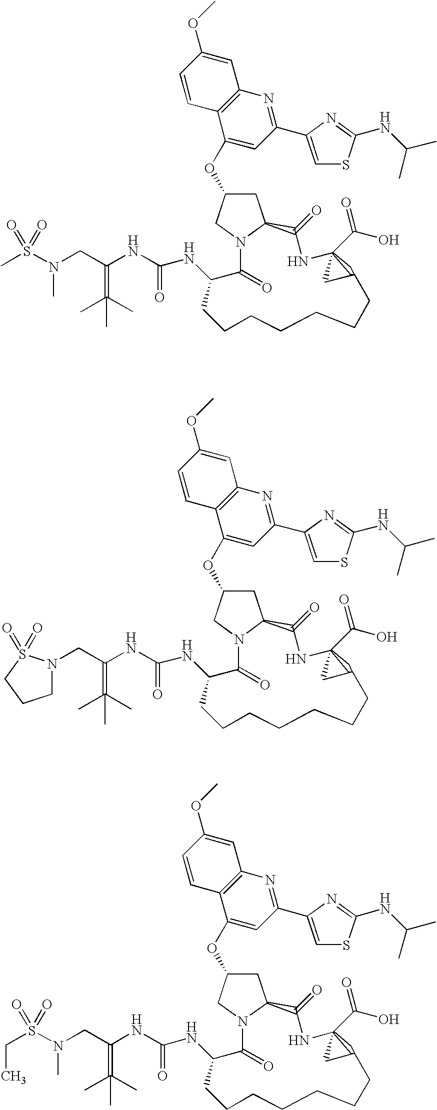 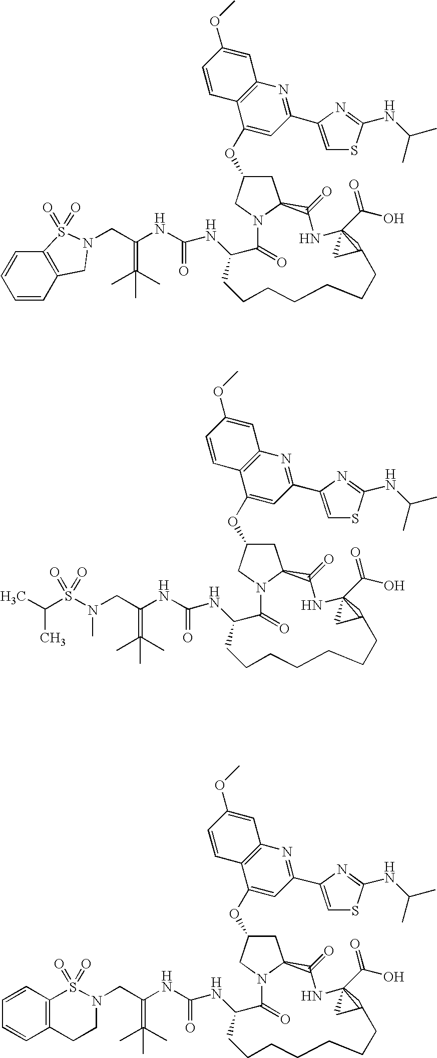 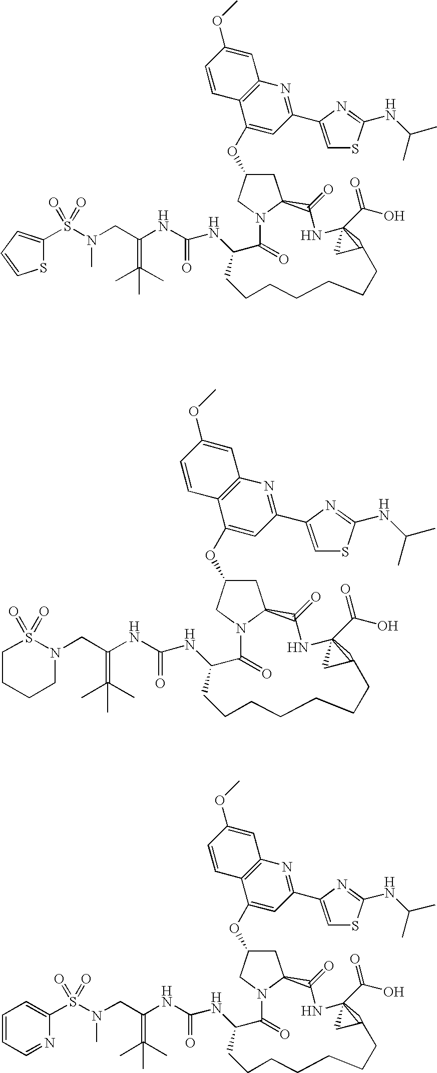 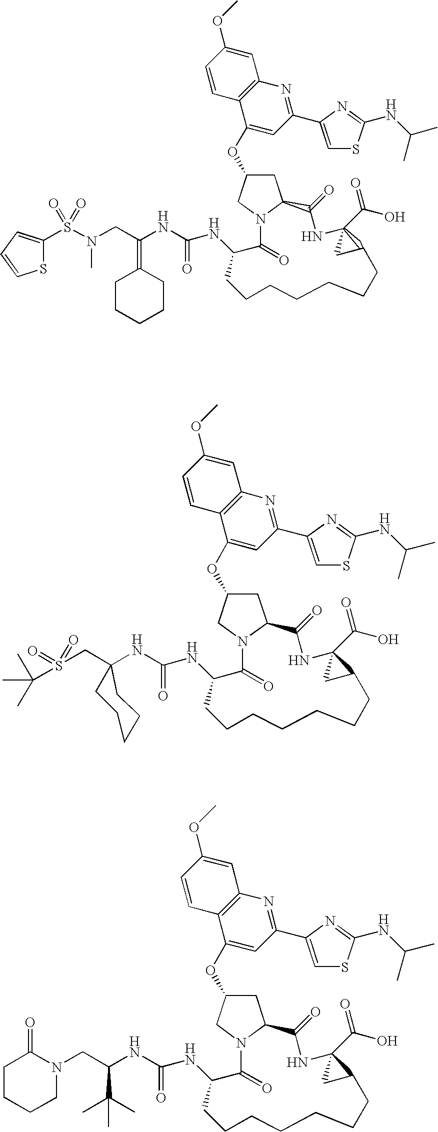 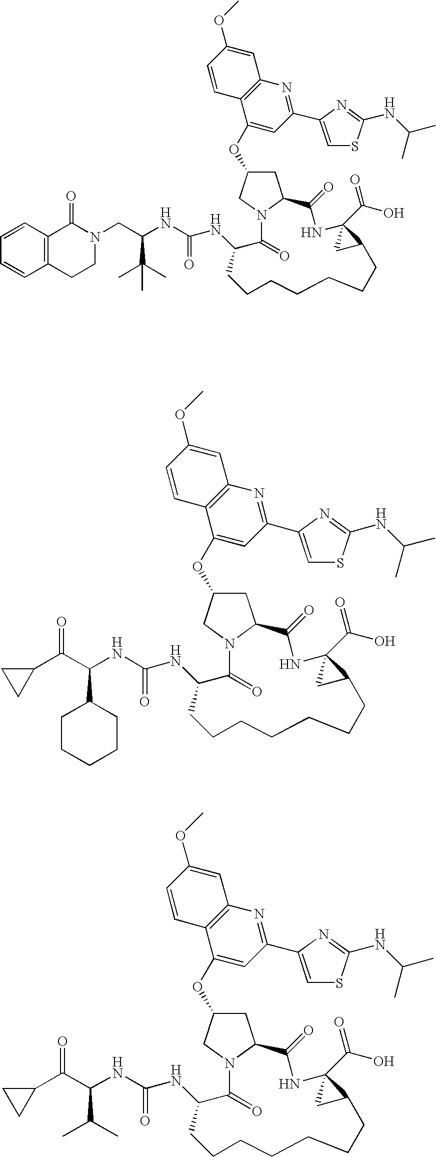 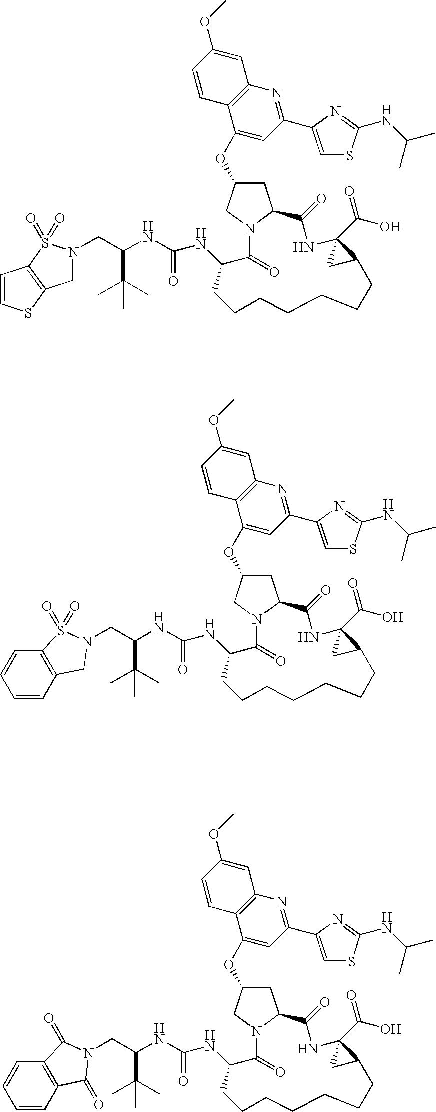 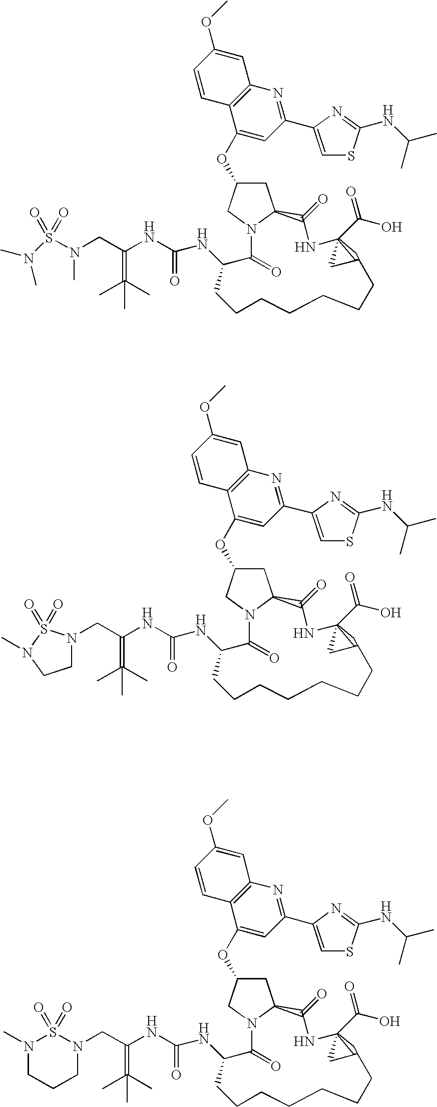 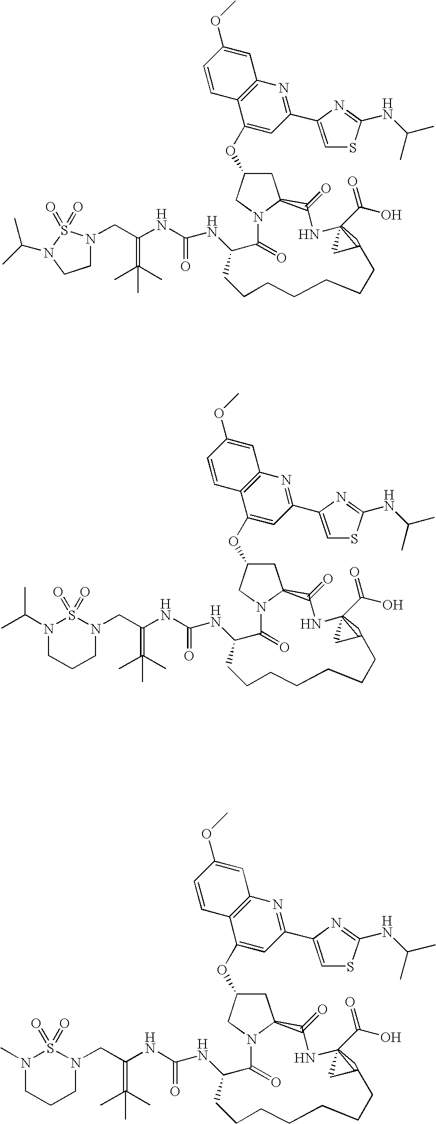 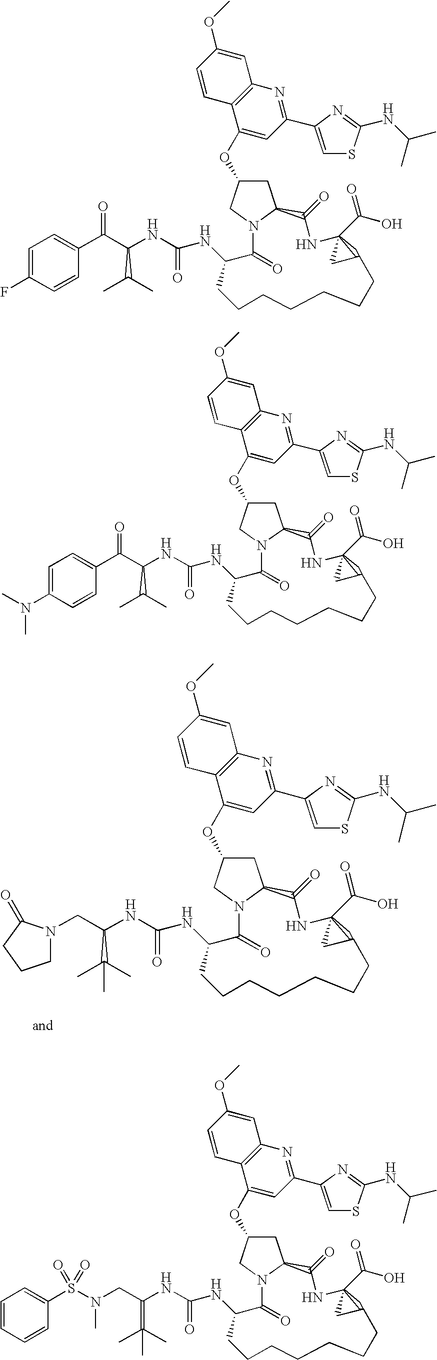
or a pharmaceutically acceptable salt, solvate or ester thereof.

Compounds of formula X are disclosed in U.S. Patent Application Publication US 2005/0267043, published Dec. 1, 1005, and the corresponding PCT publication WO2005/085275, published Sep. 15, 2005, both of which are incorporated herein by reference.

Non-limiting examples of certain compounds disclosed in these publications are: 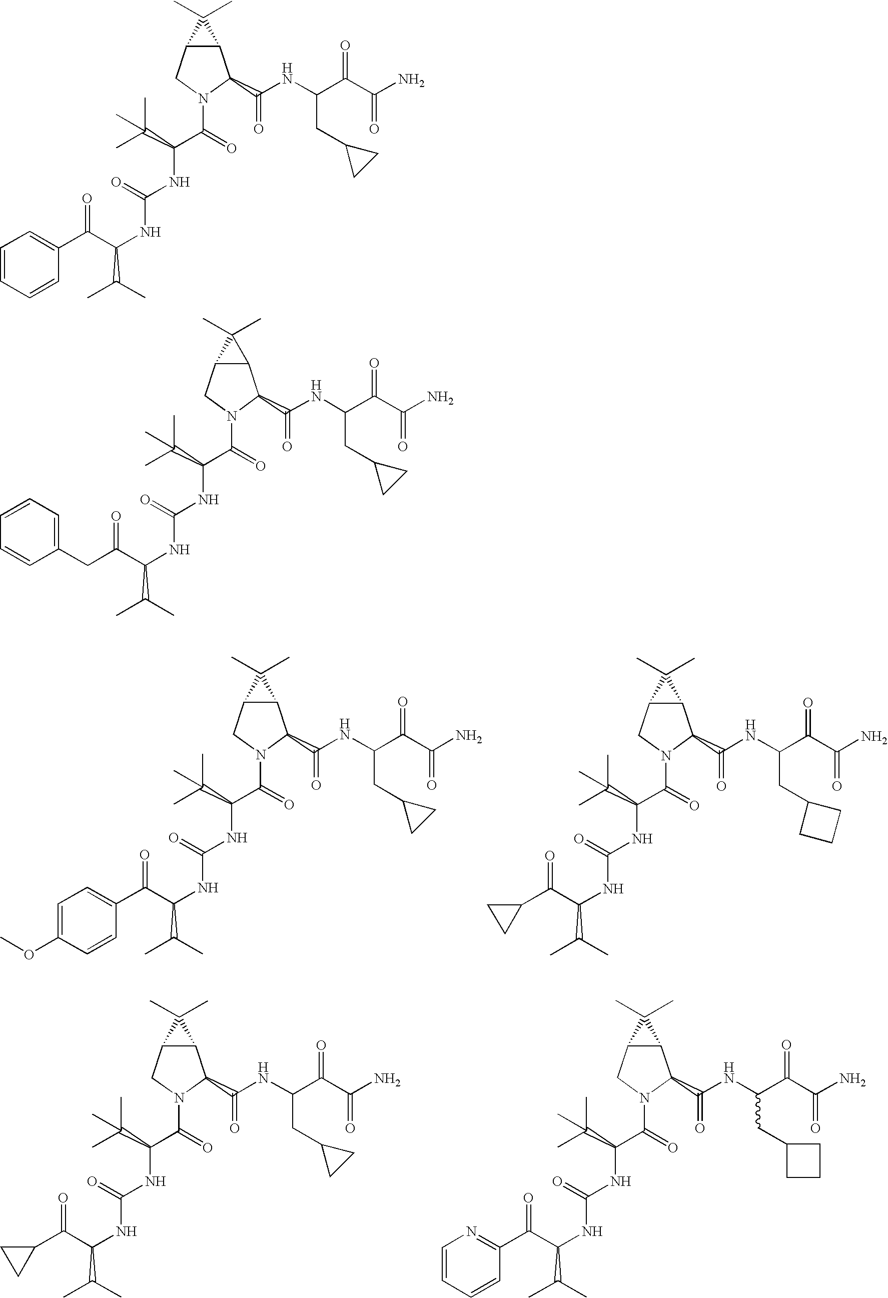 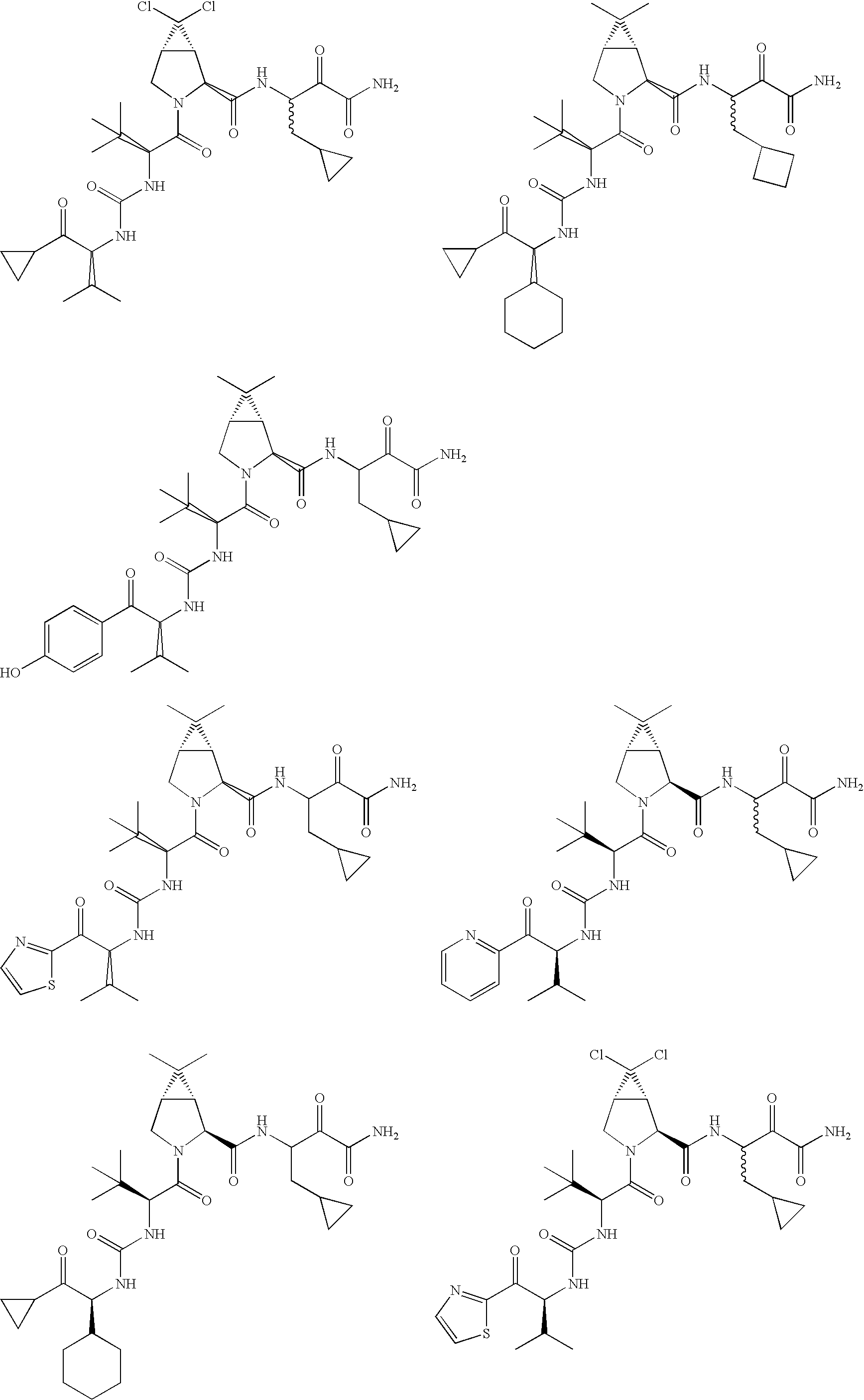 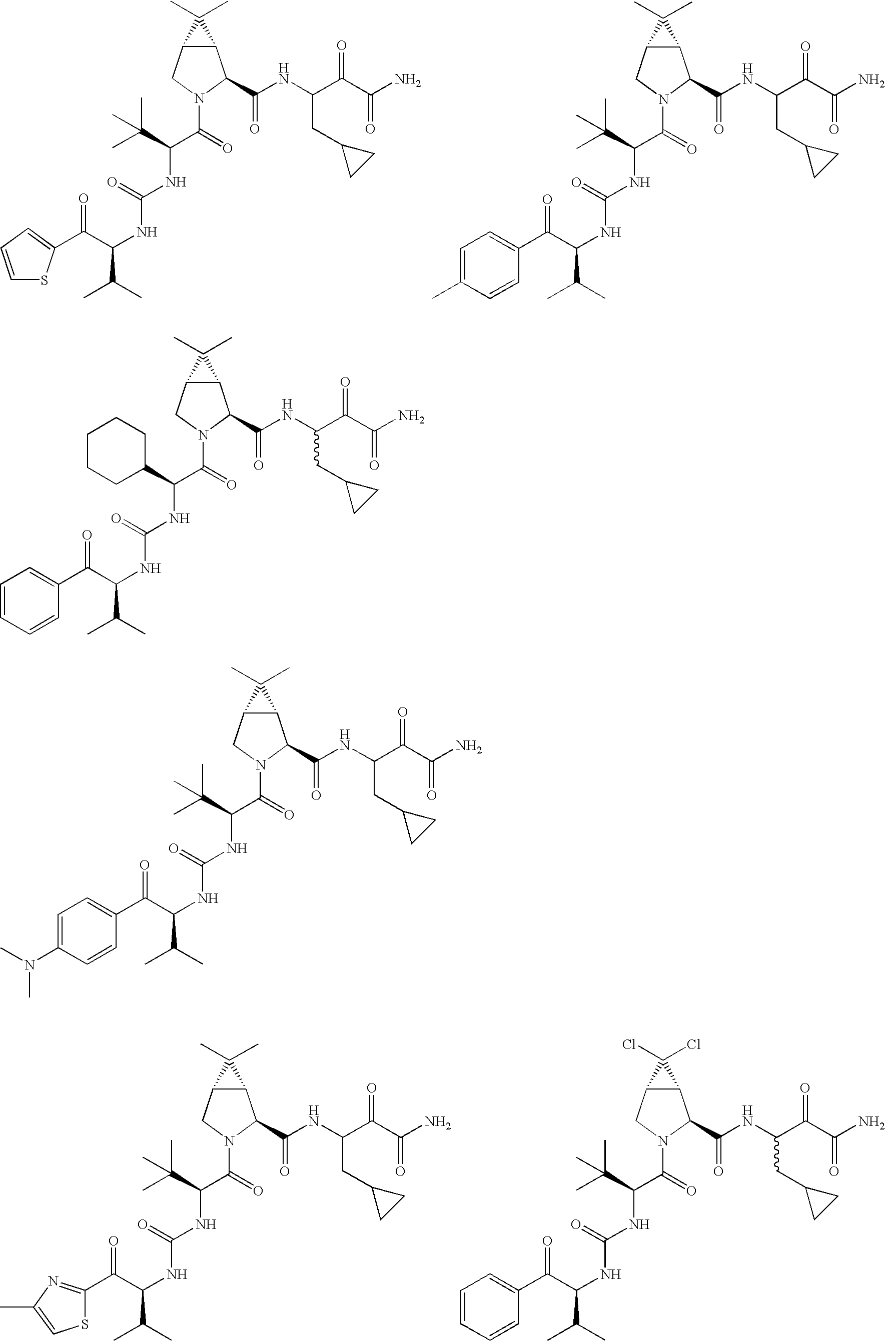 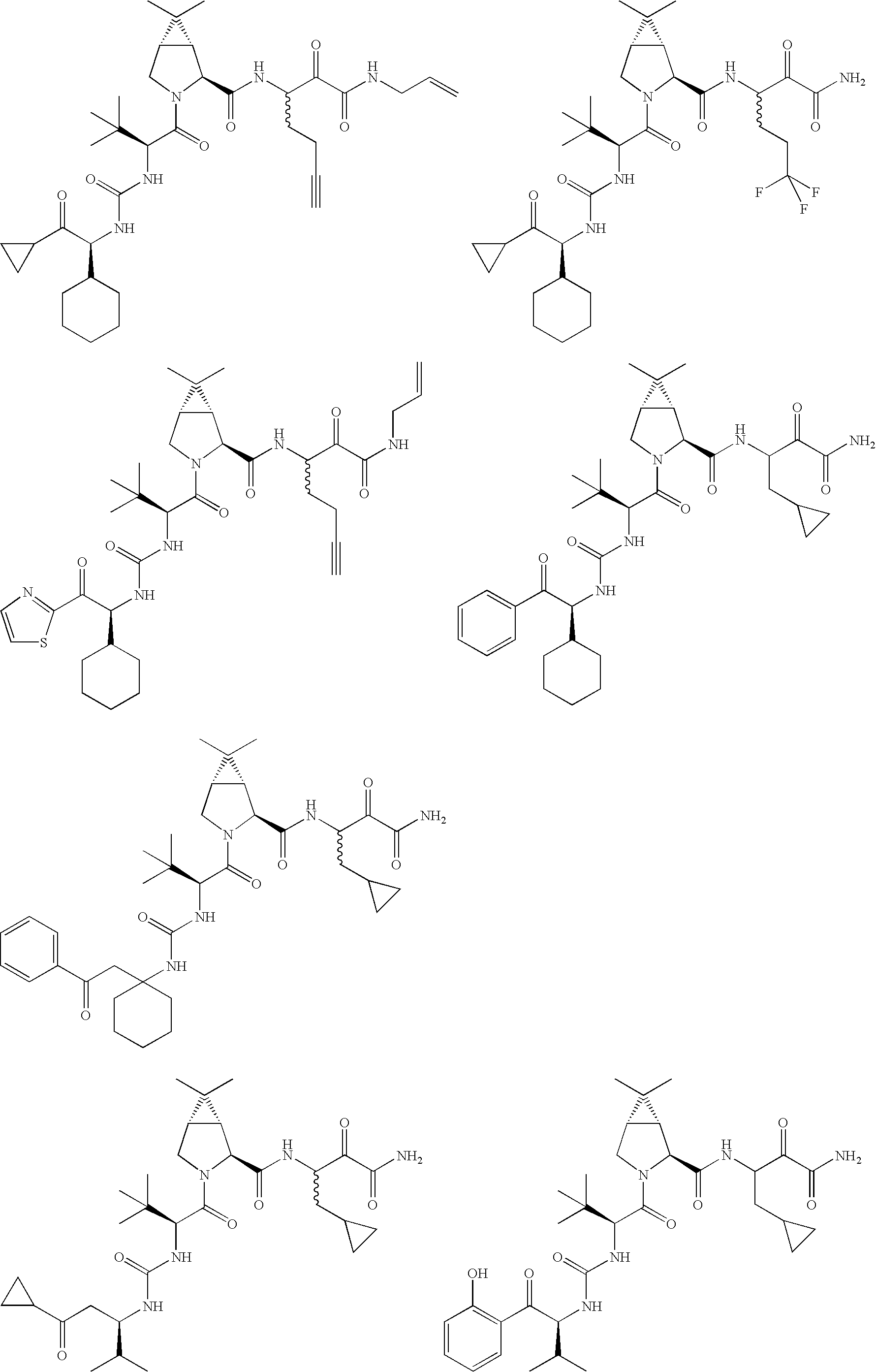 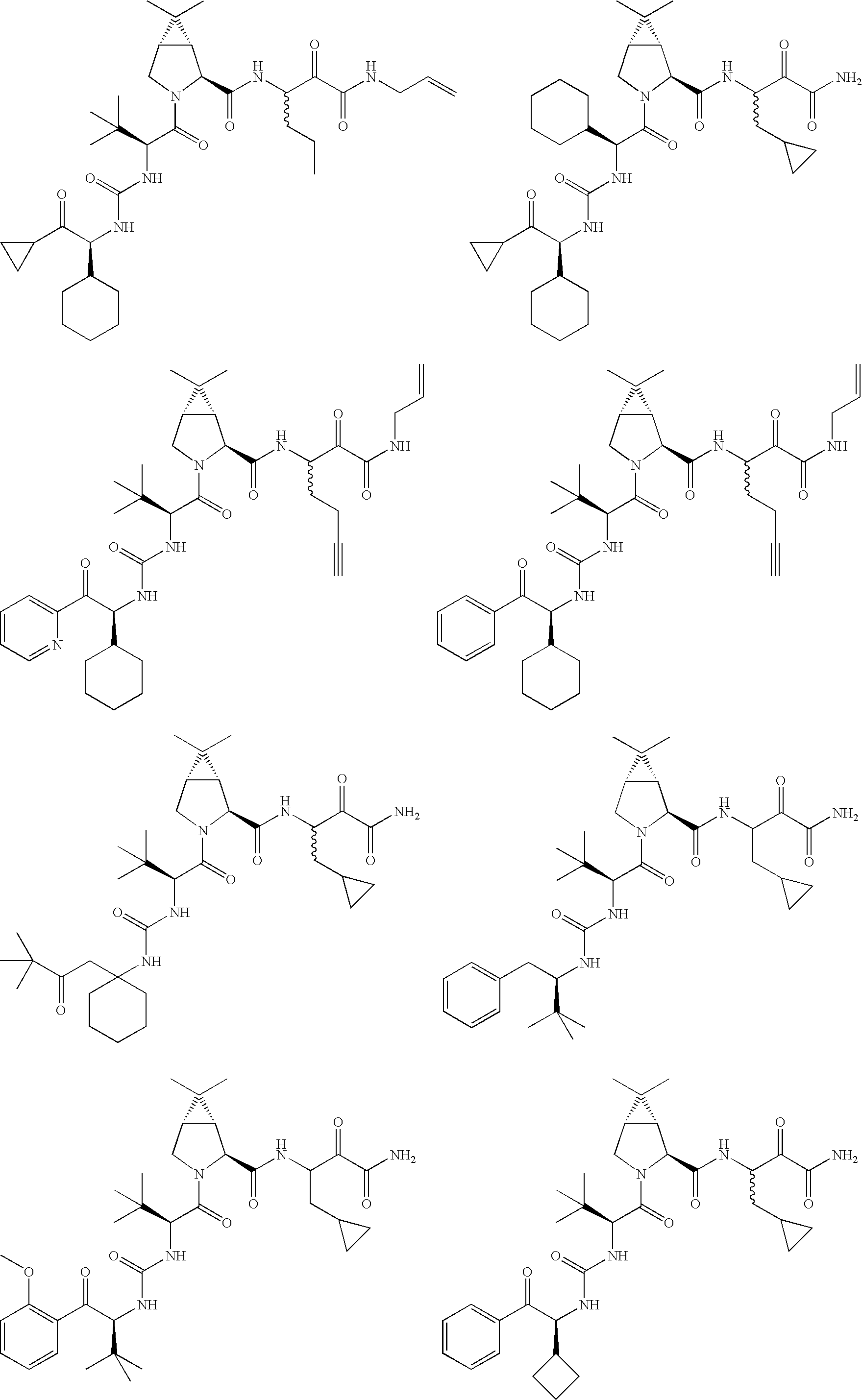 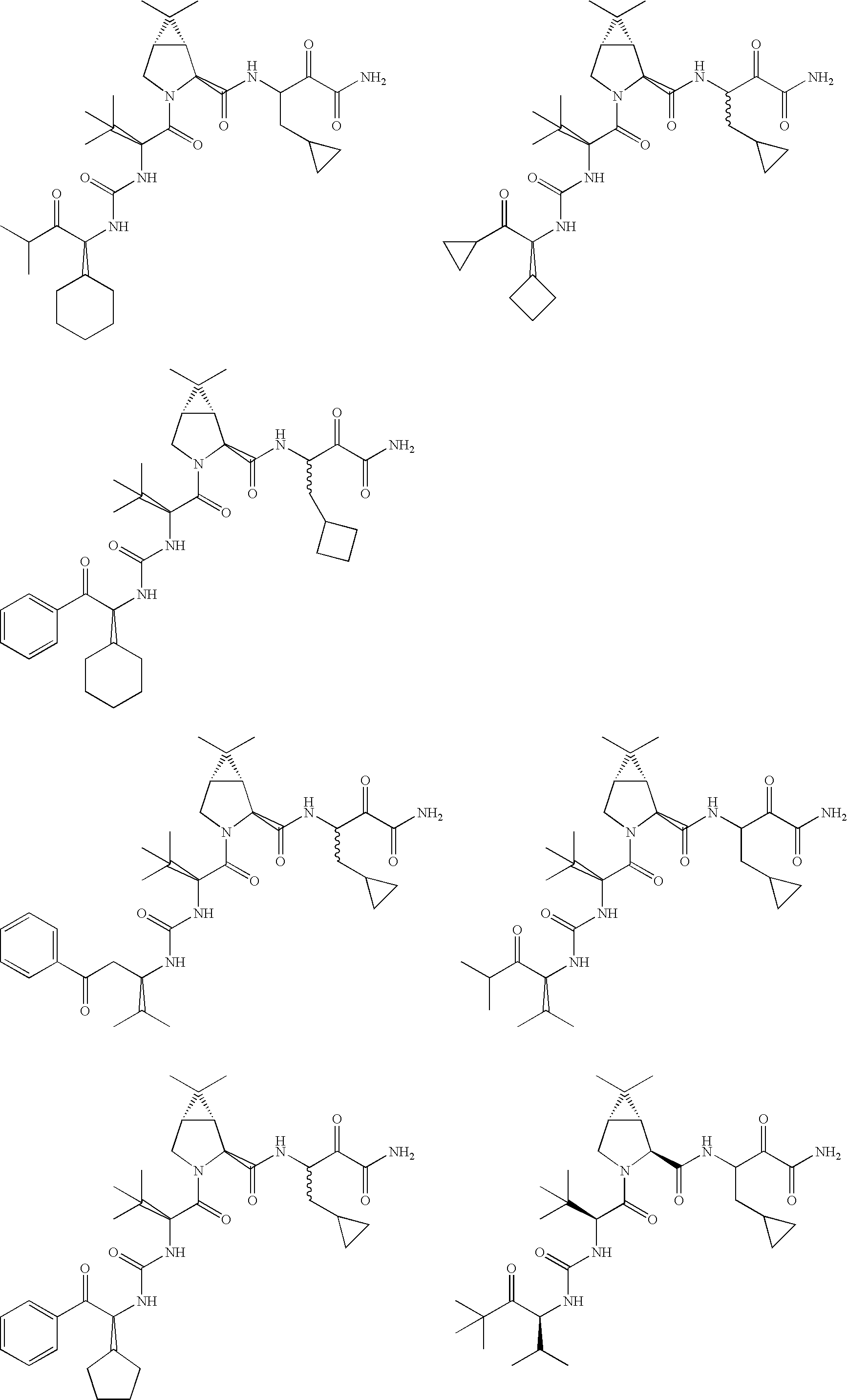 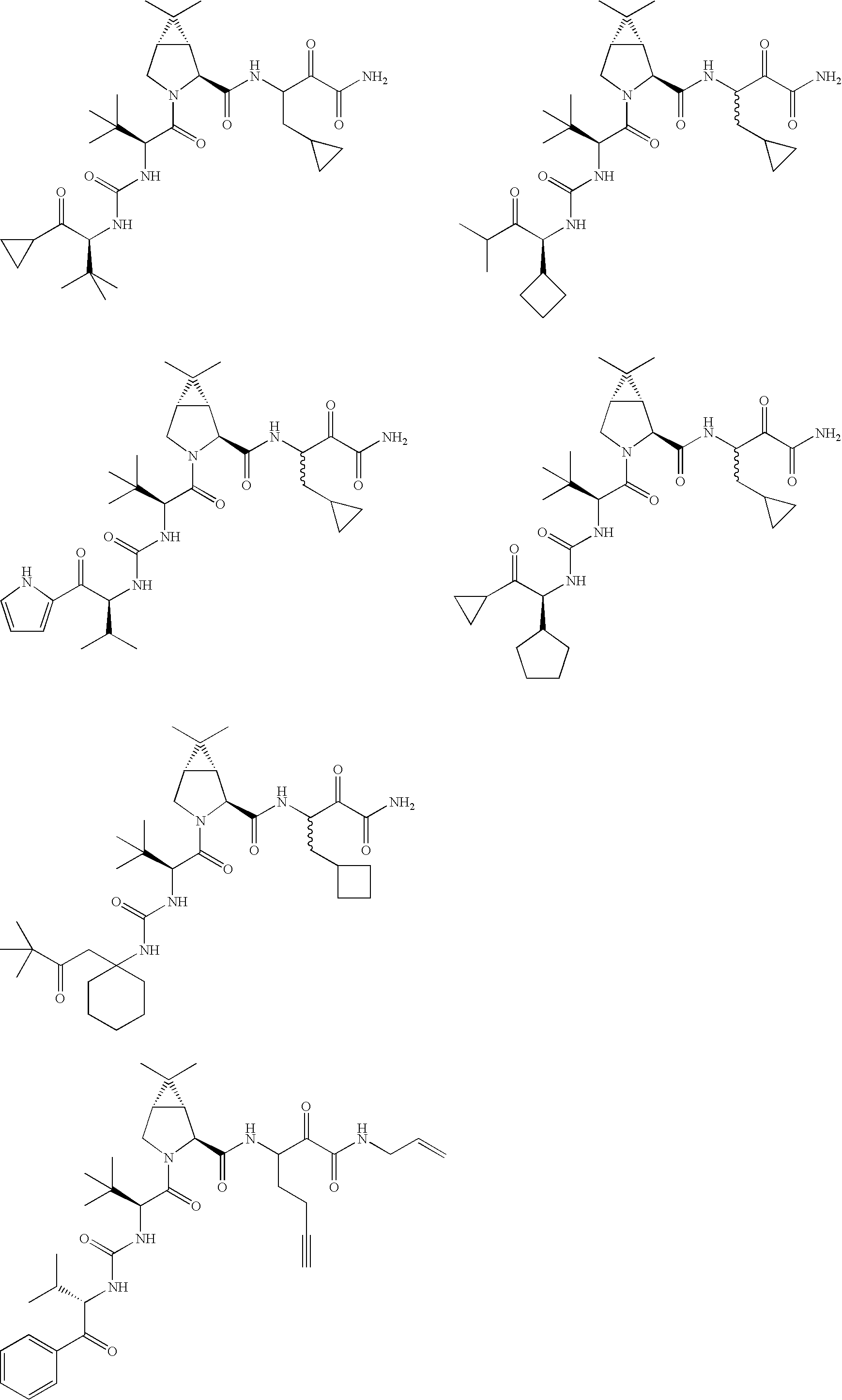 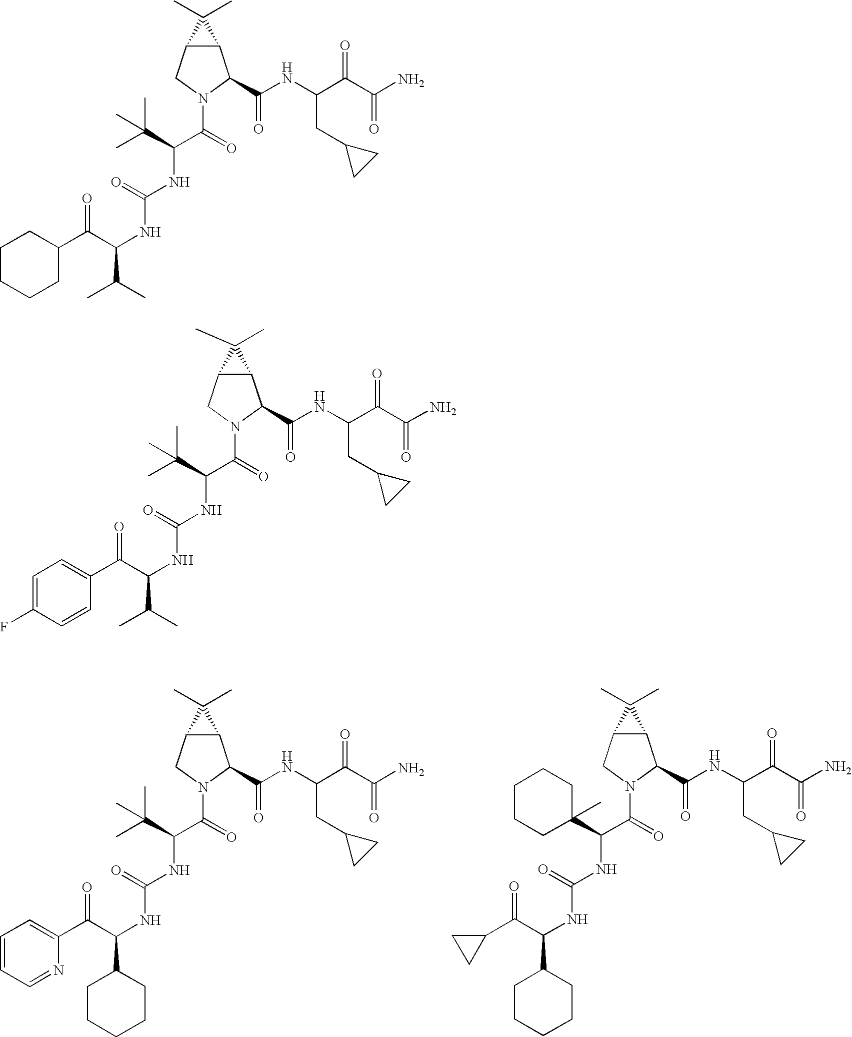 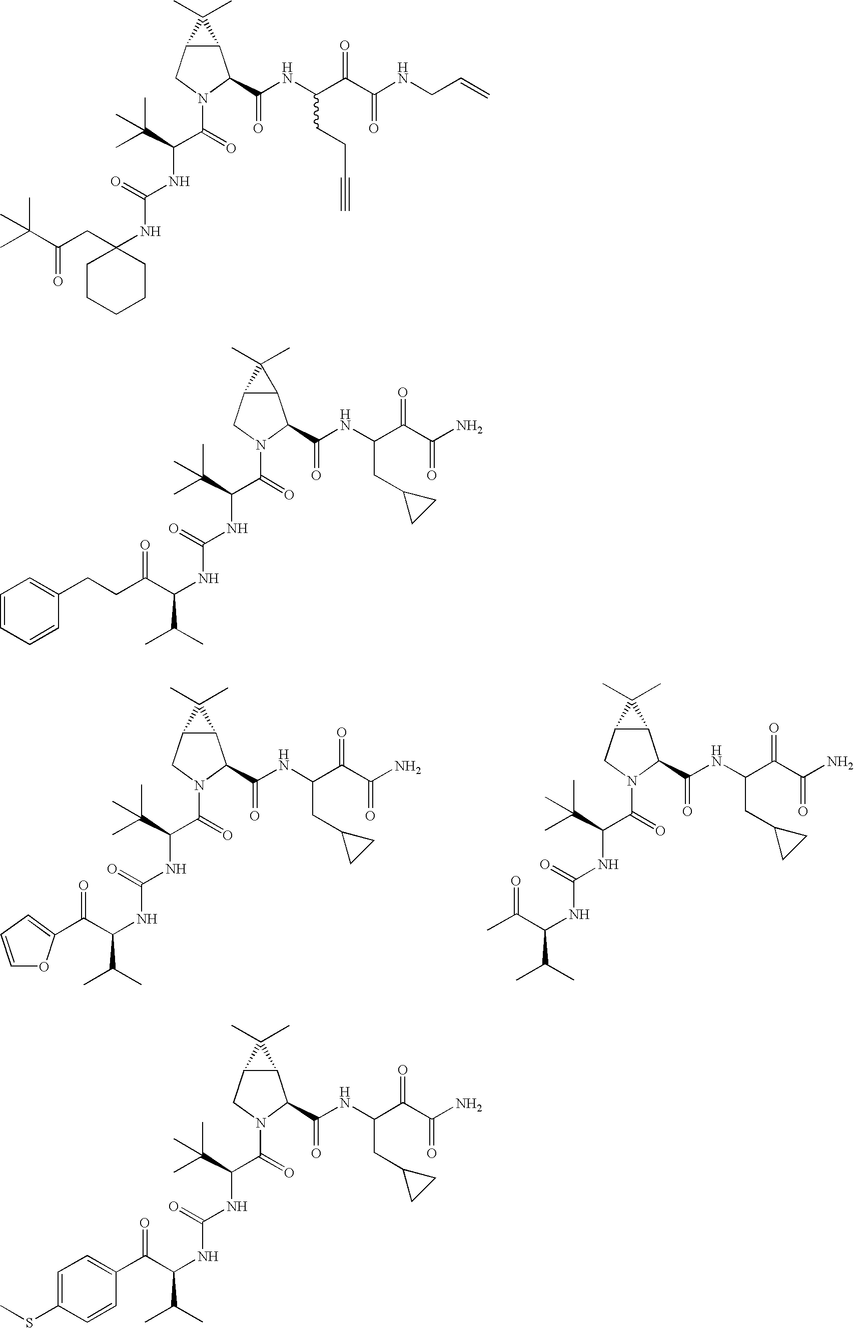 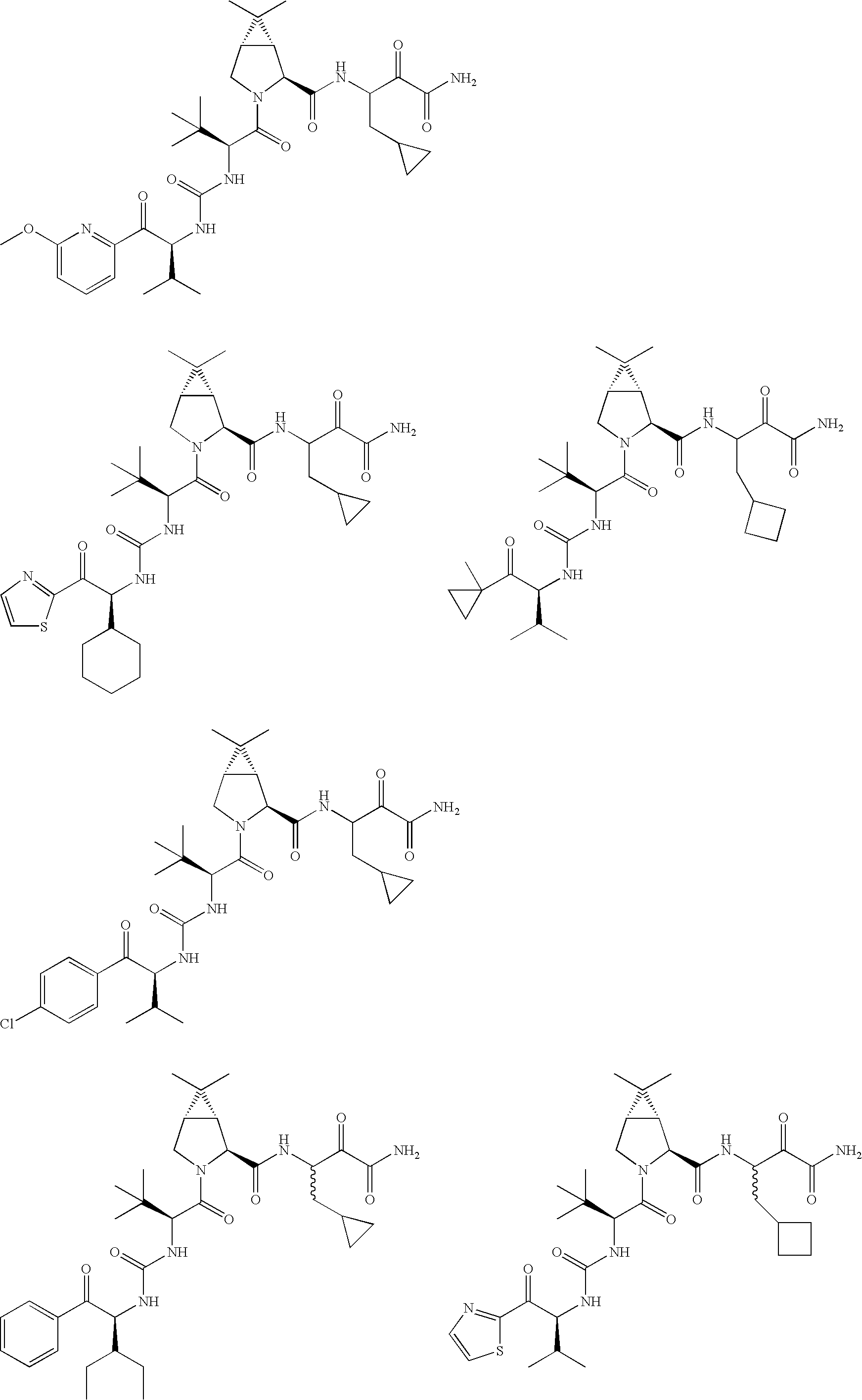 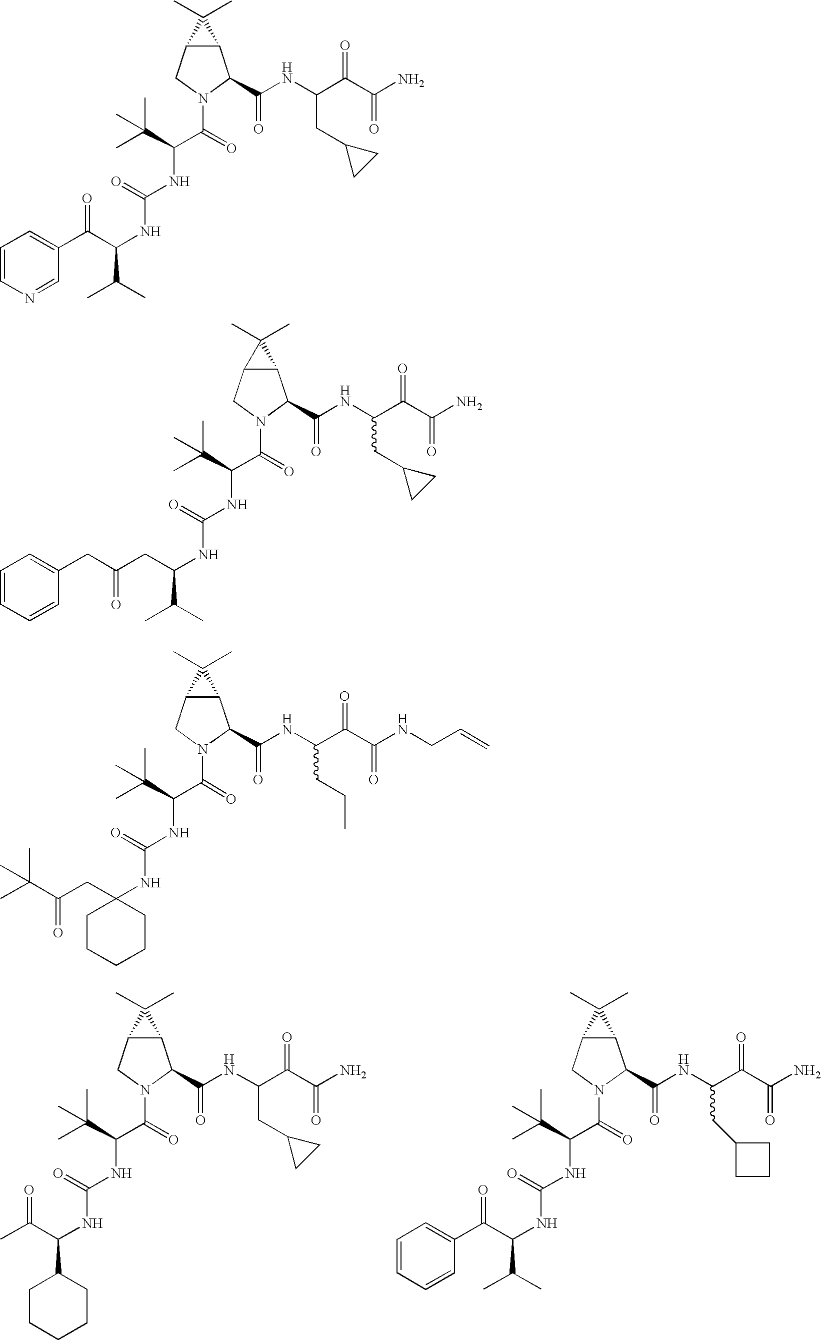 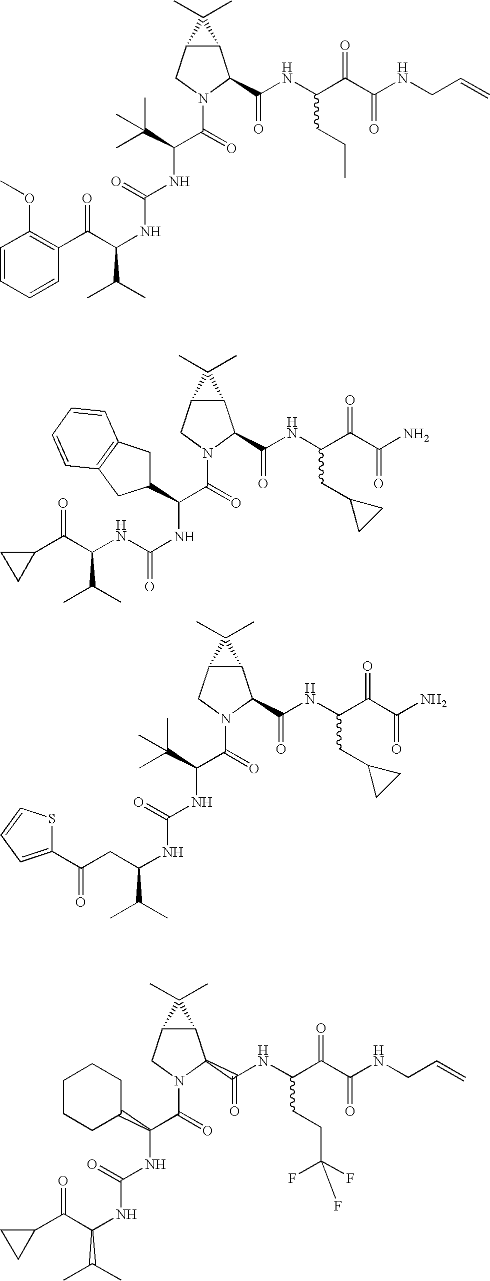 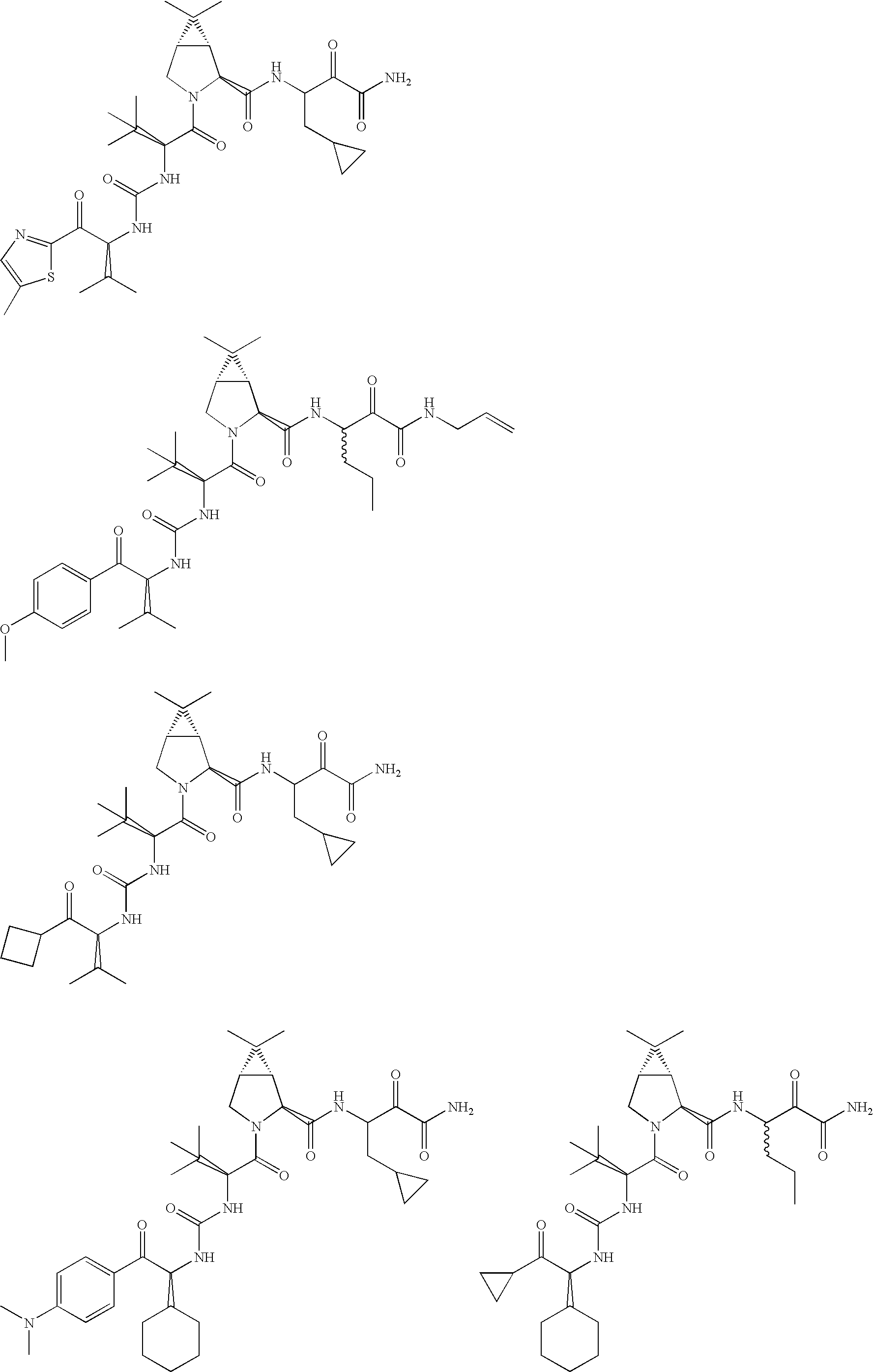 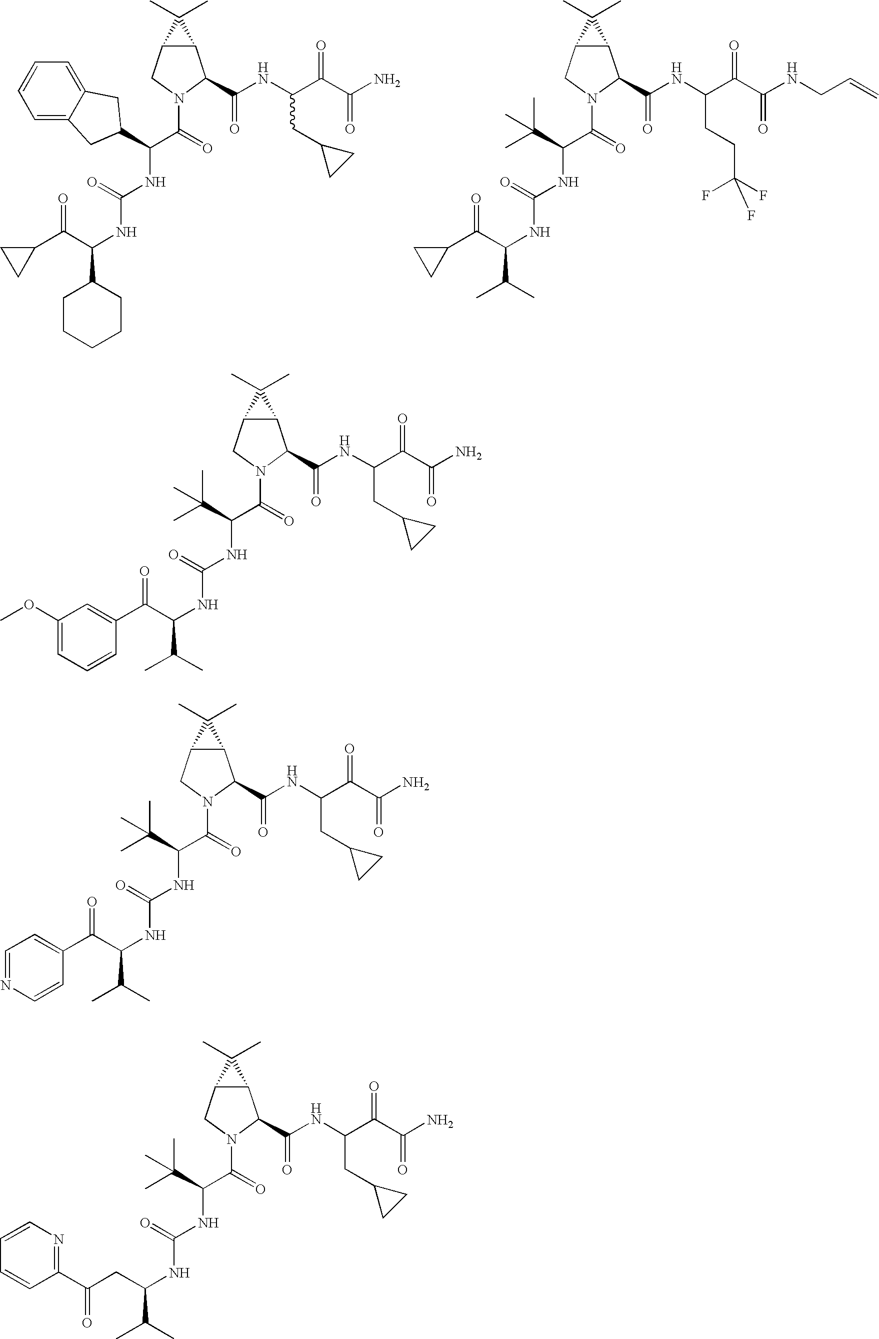 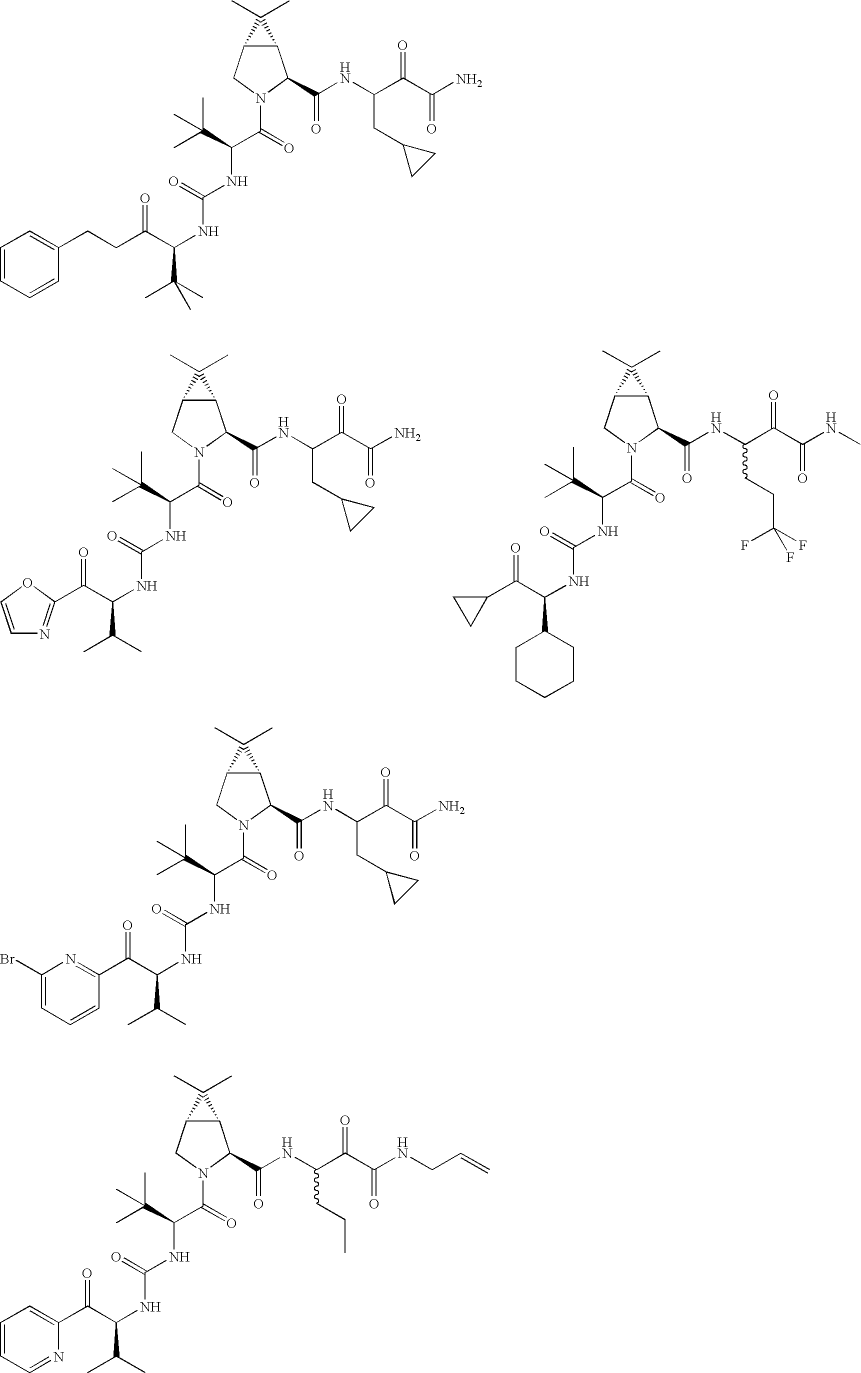 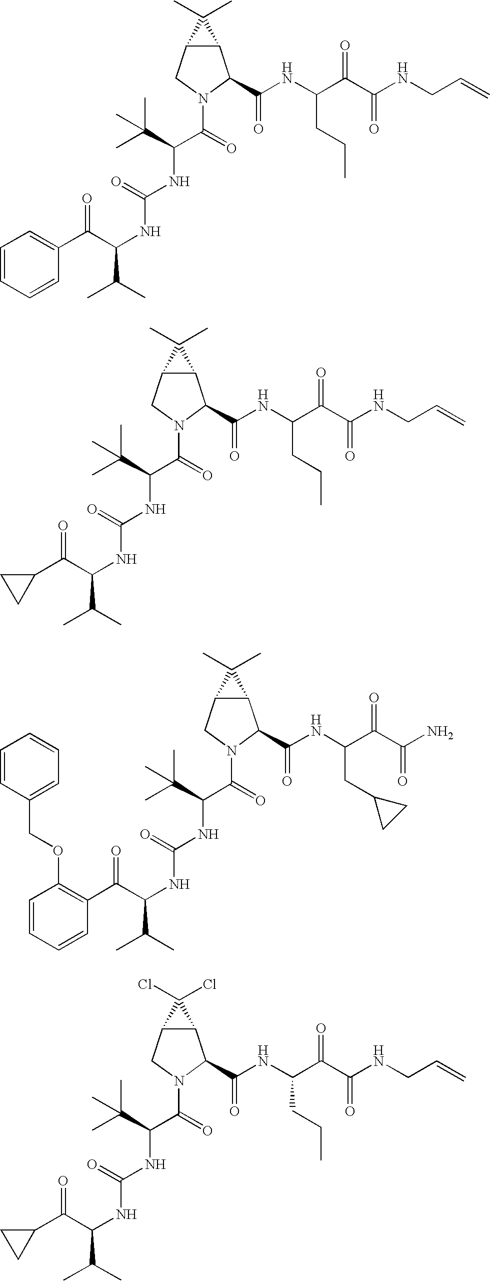 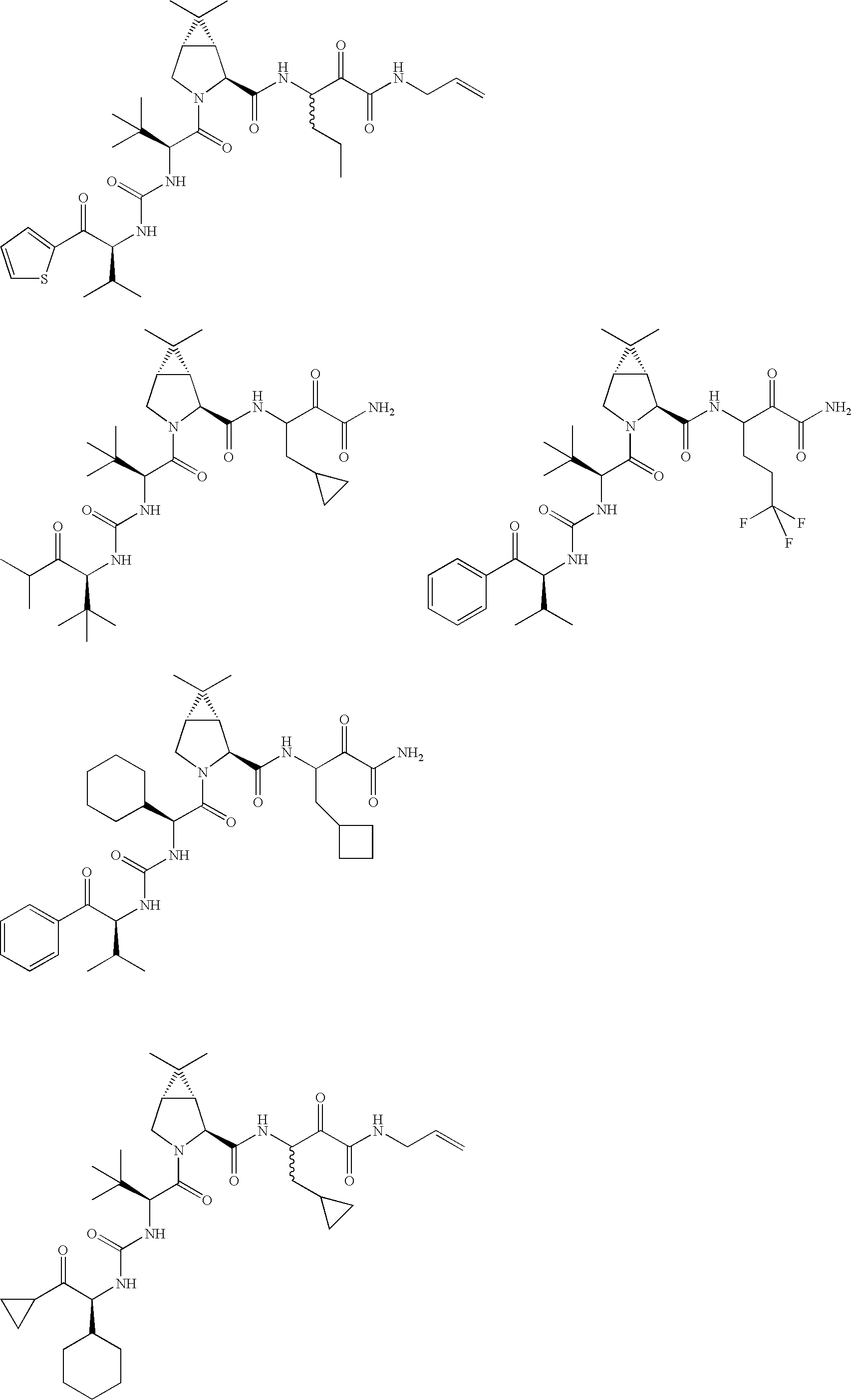 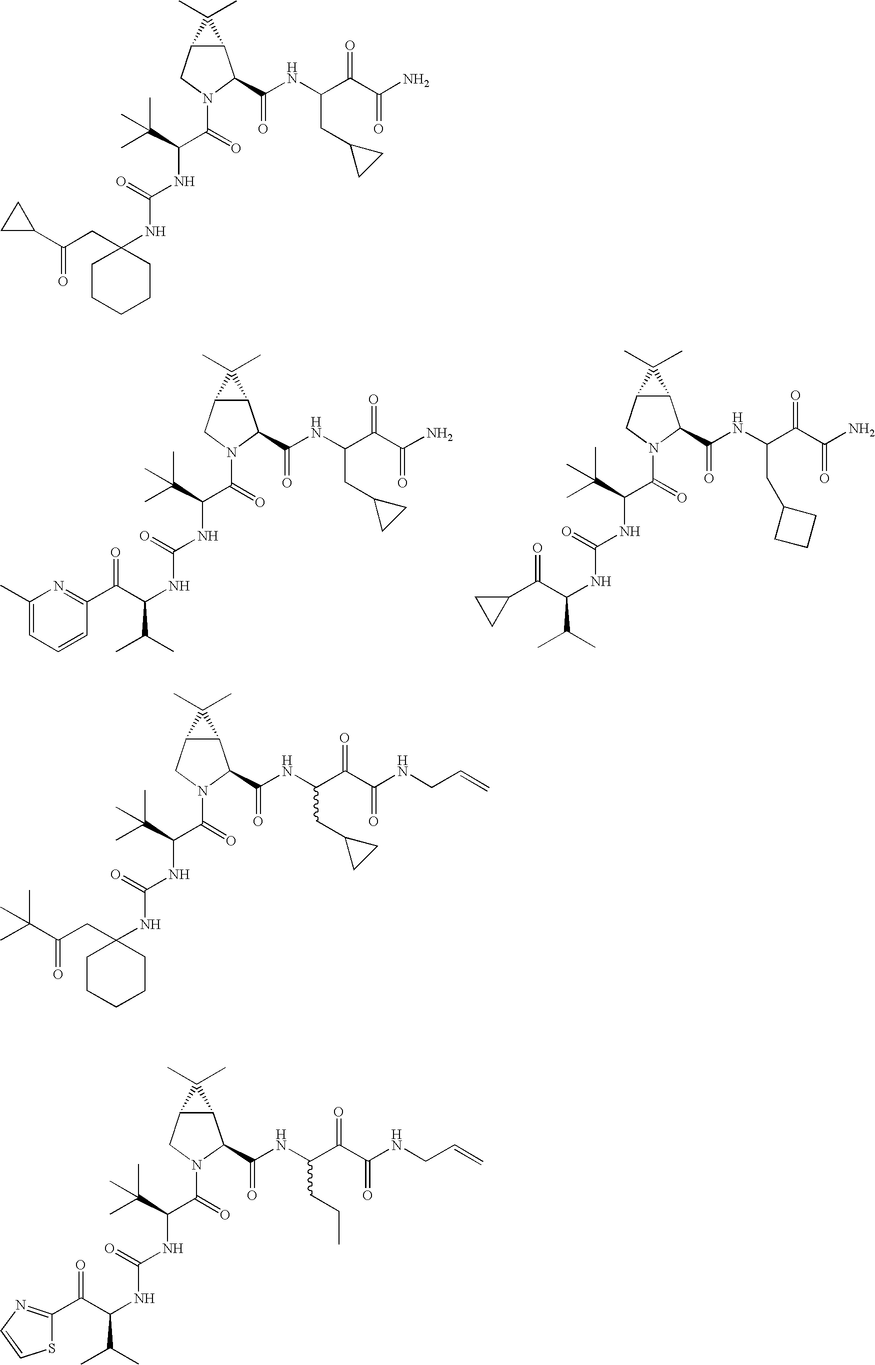 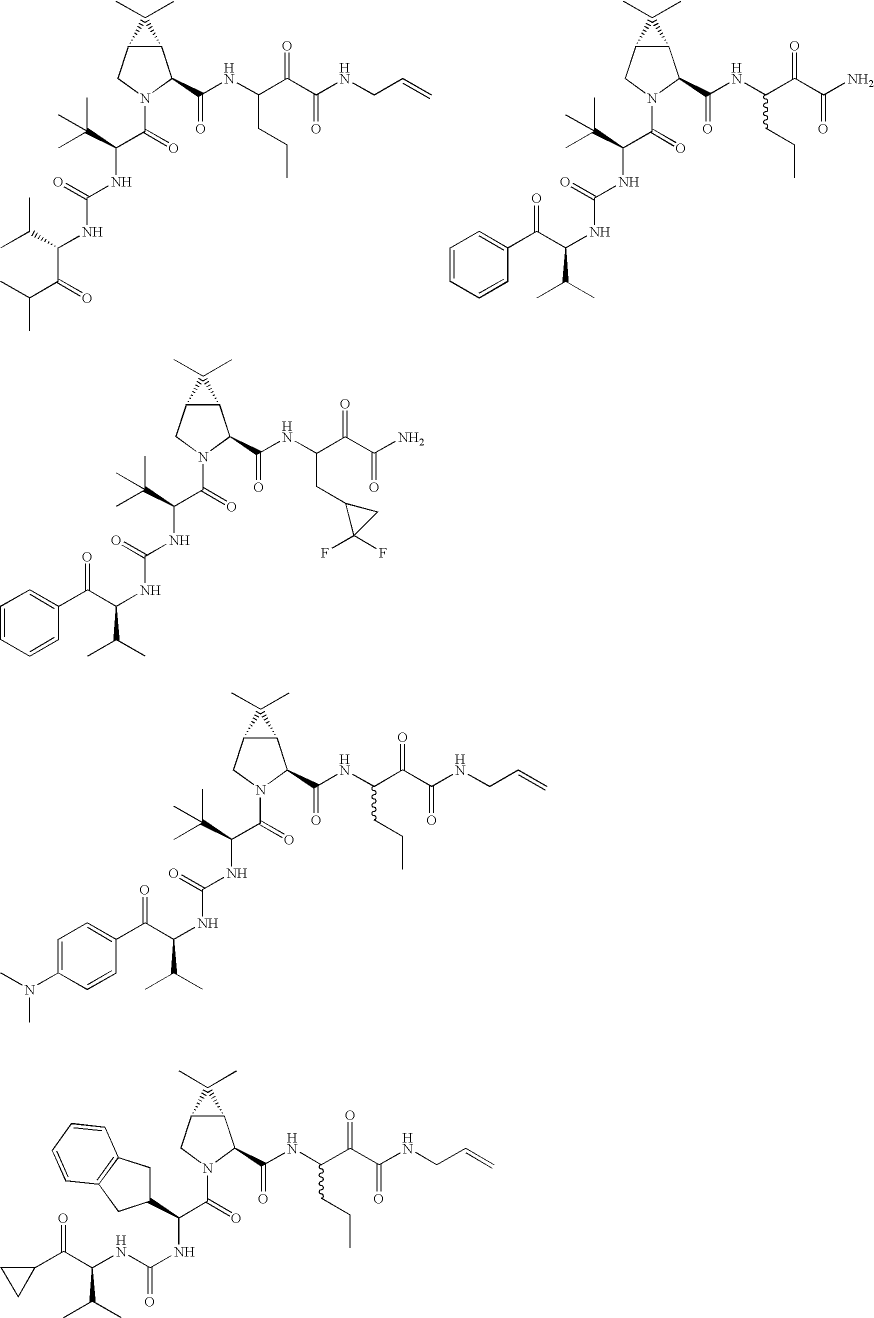 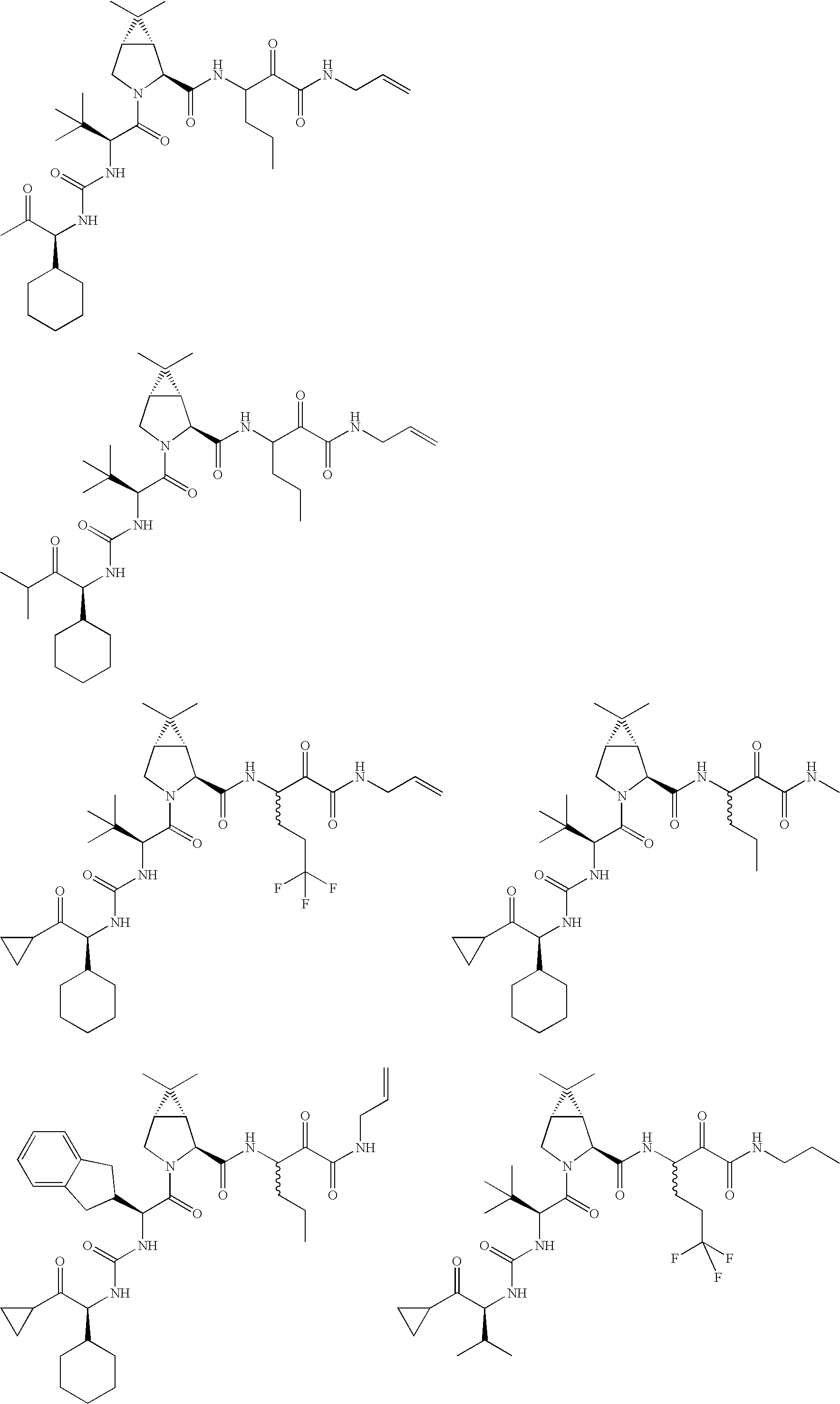 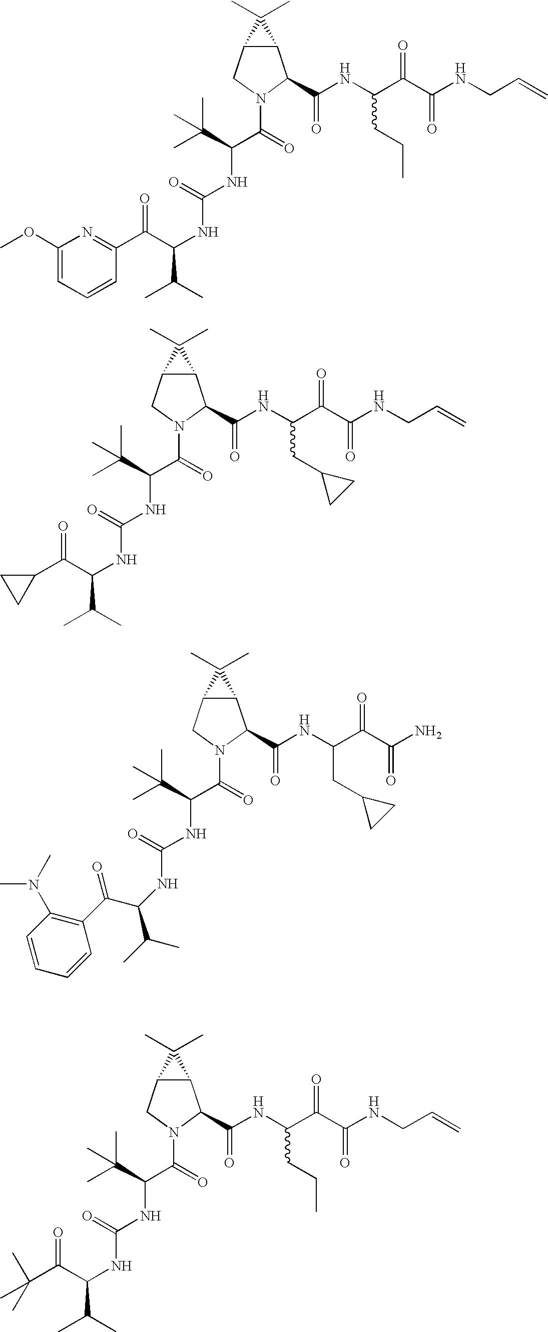 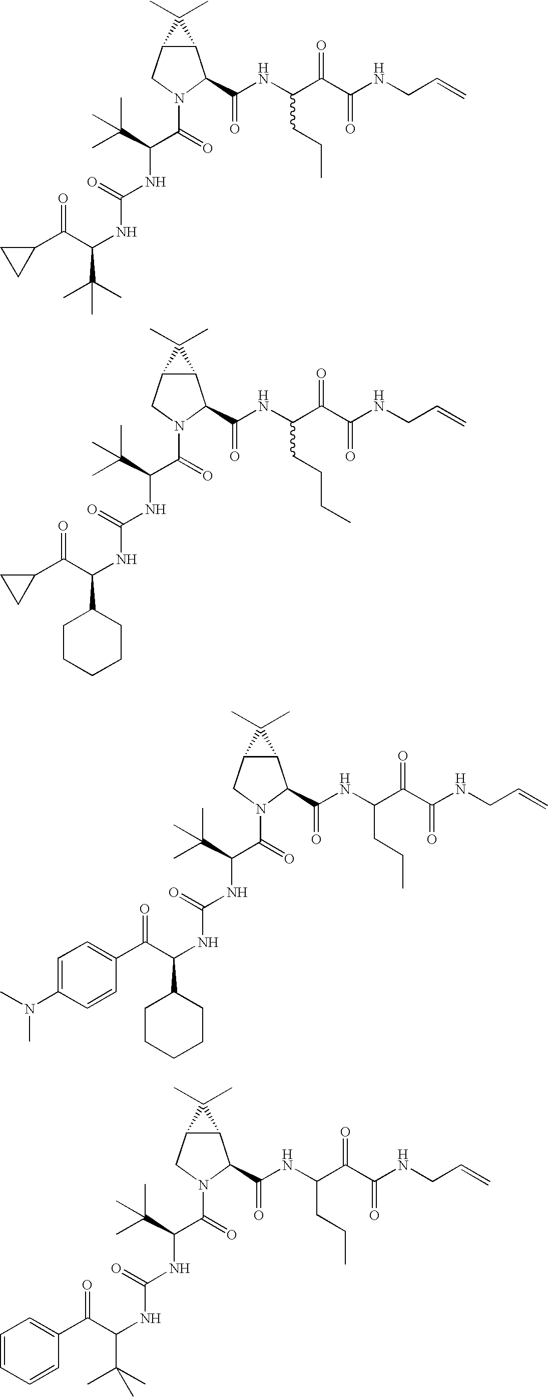 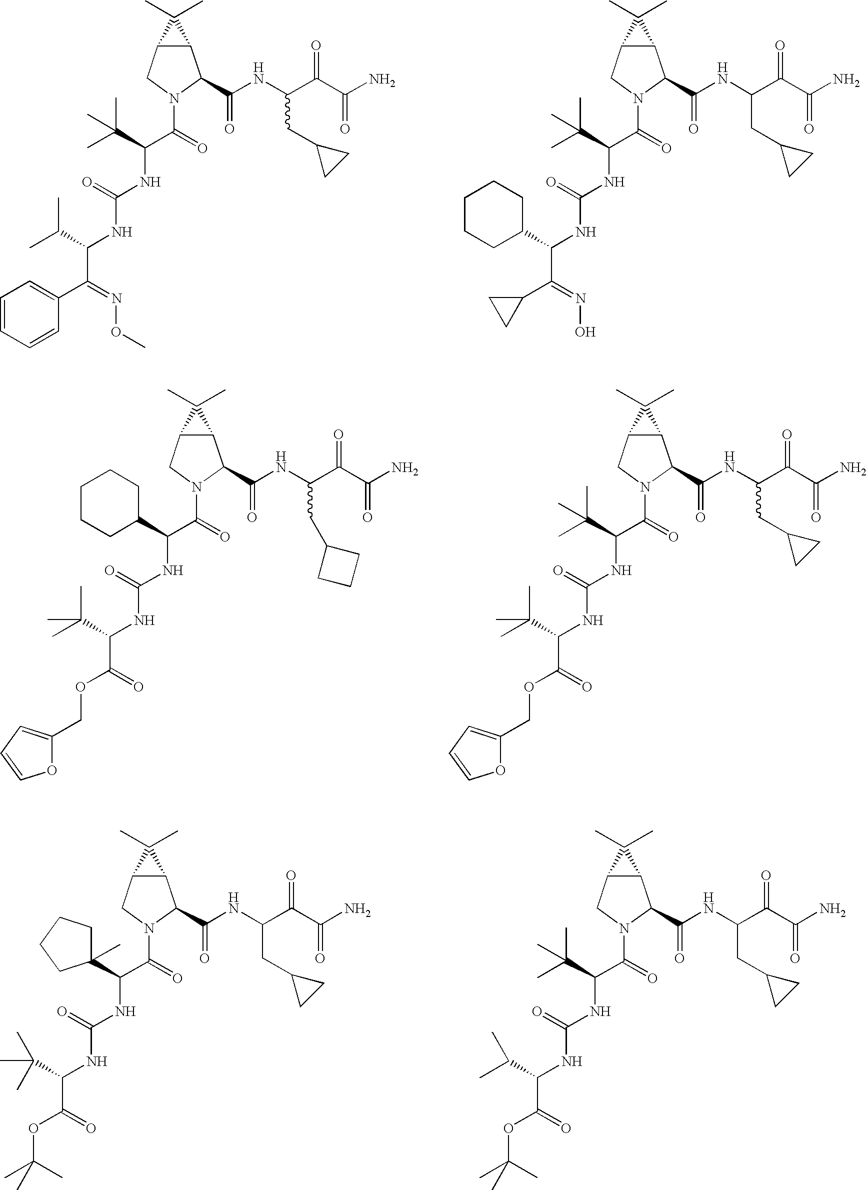 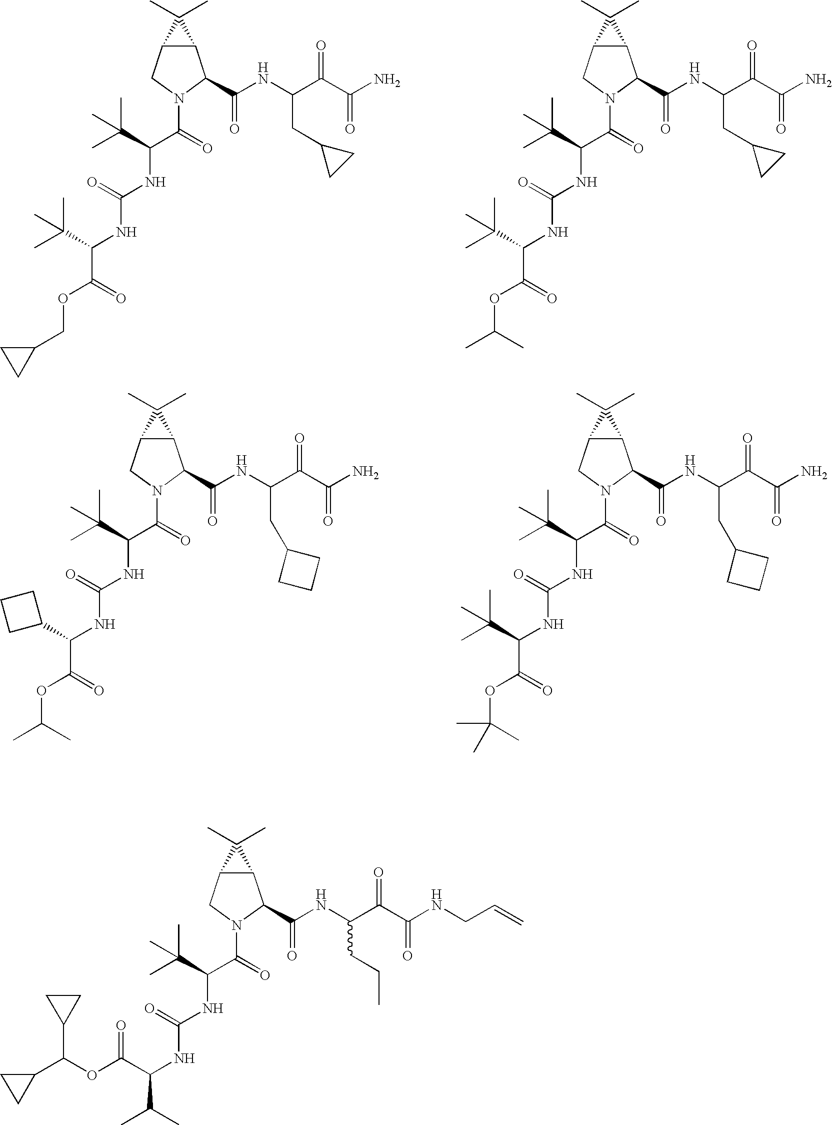 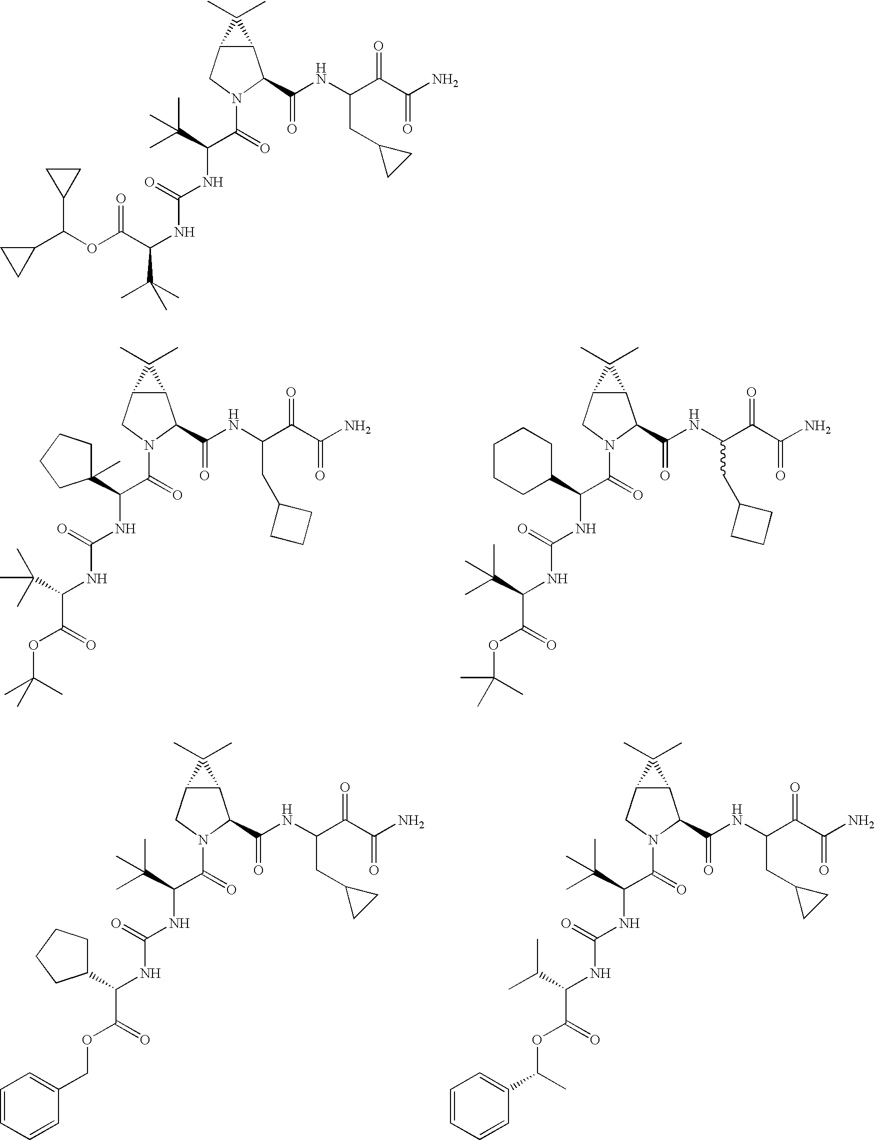 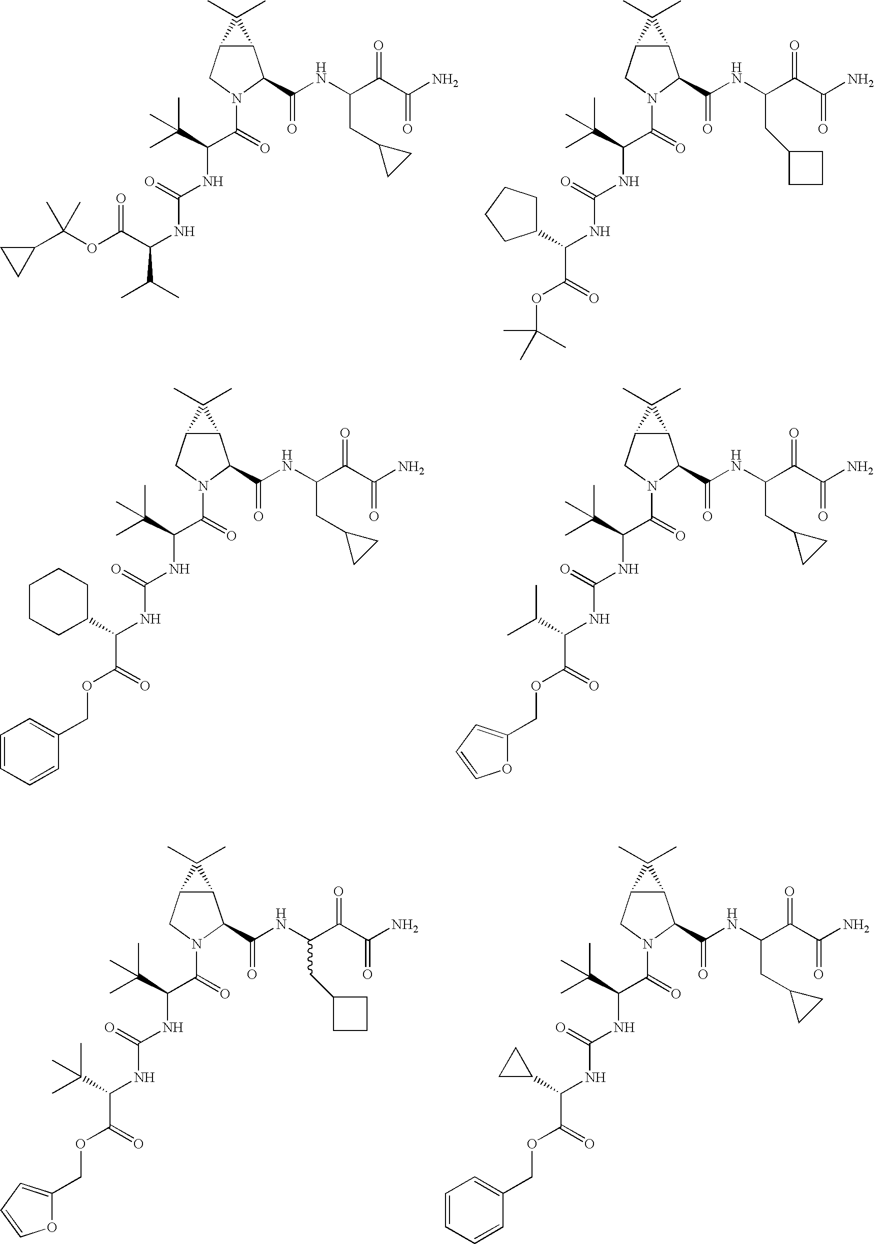 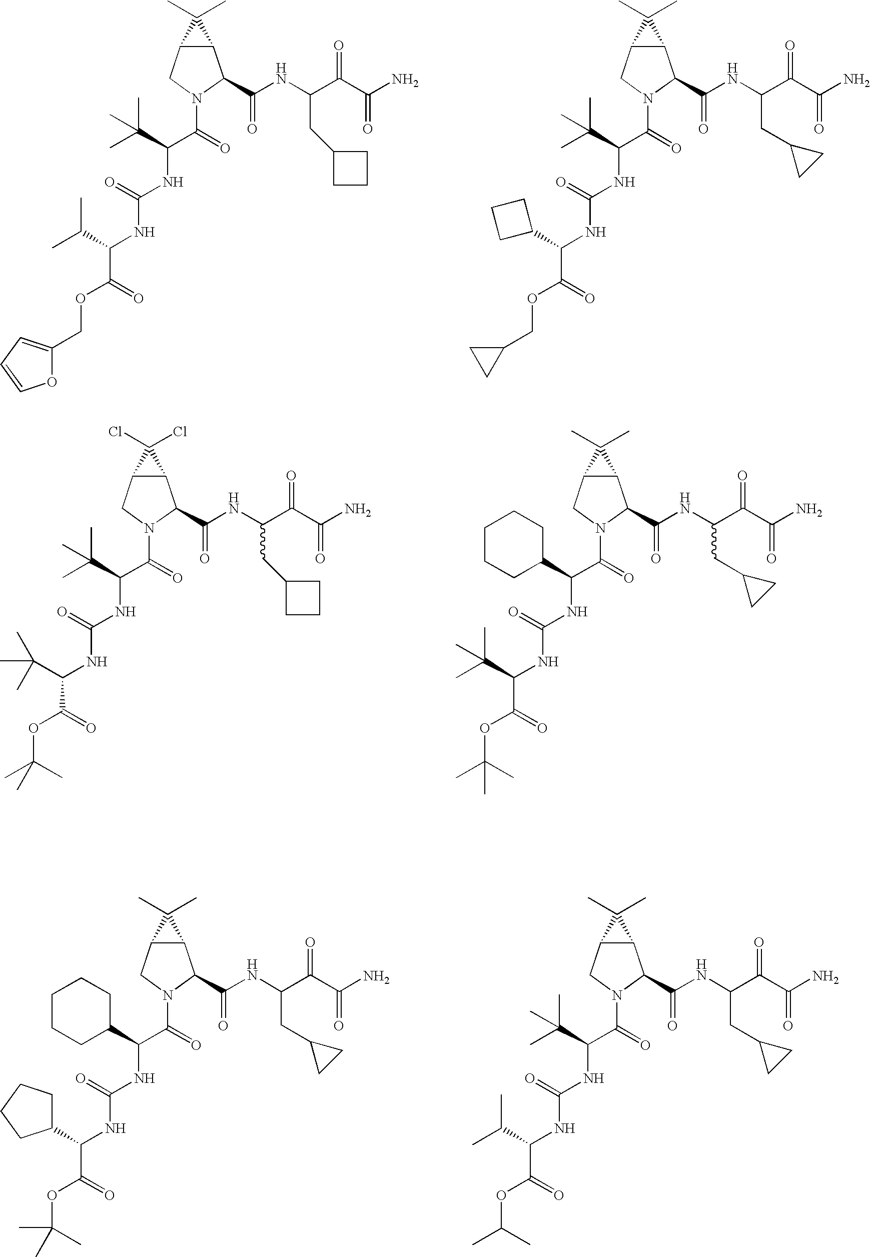 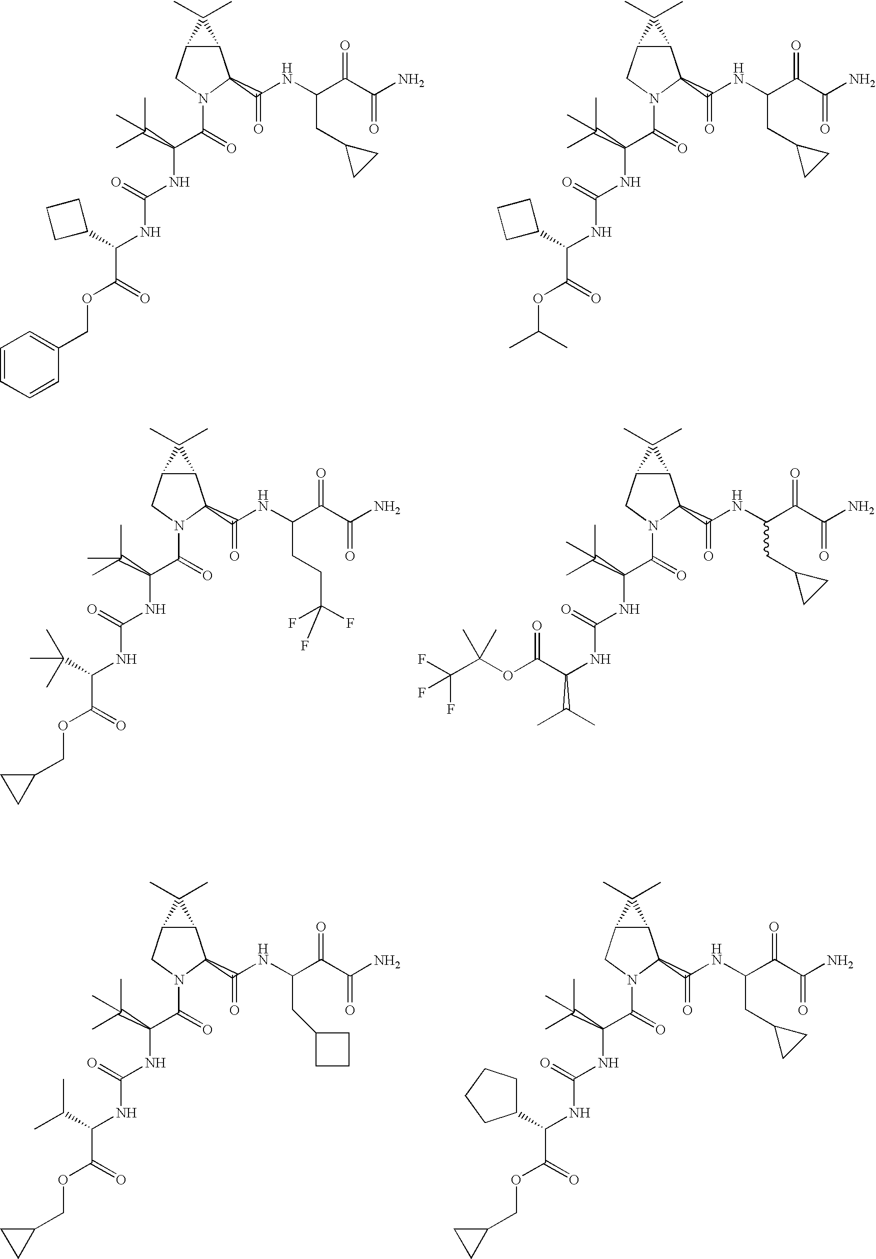 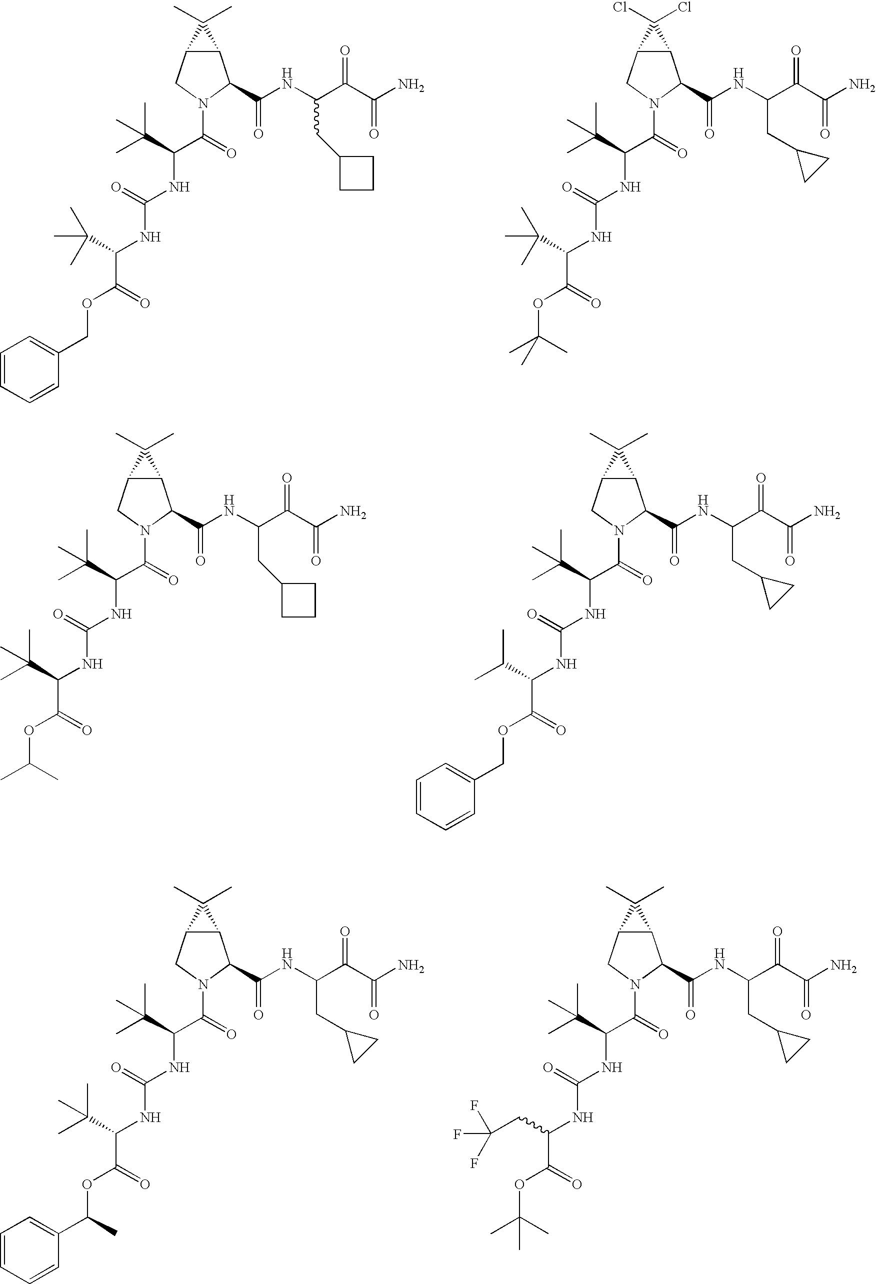 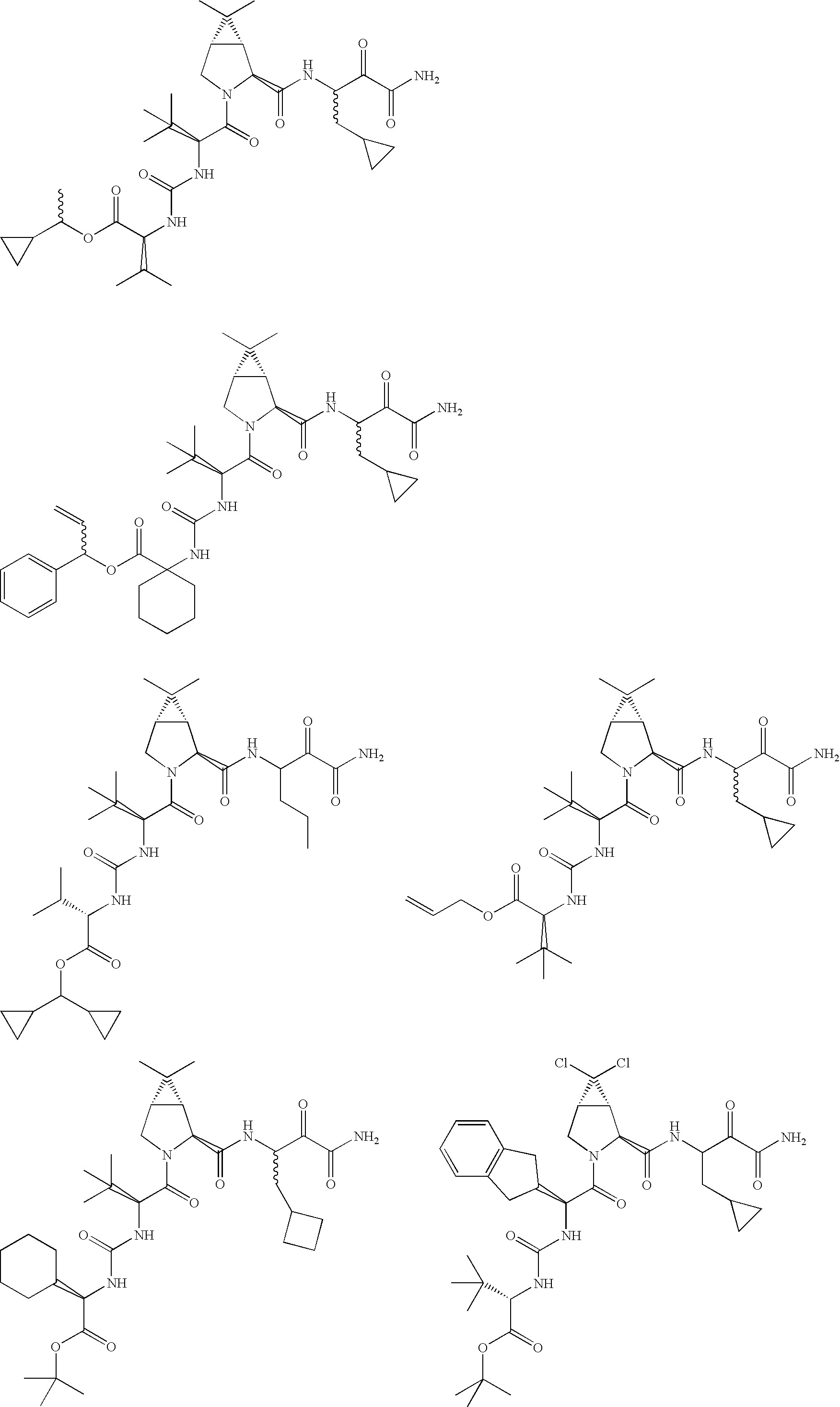 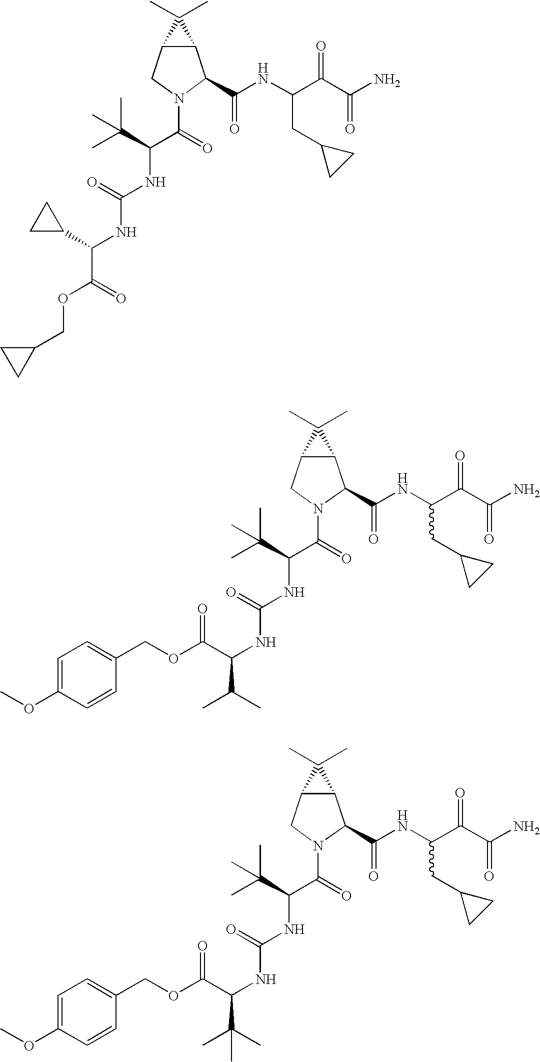 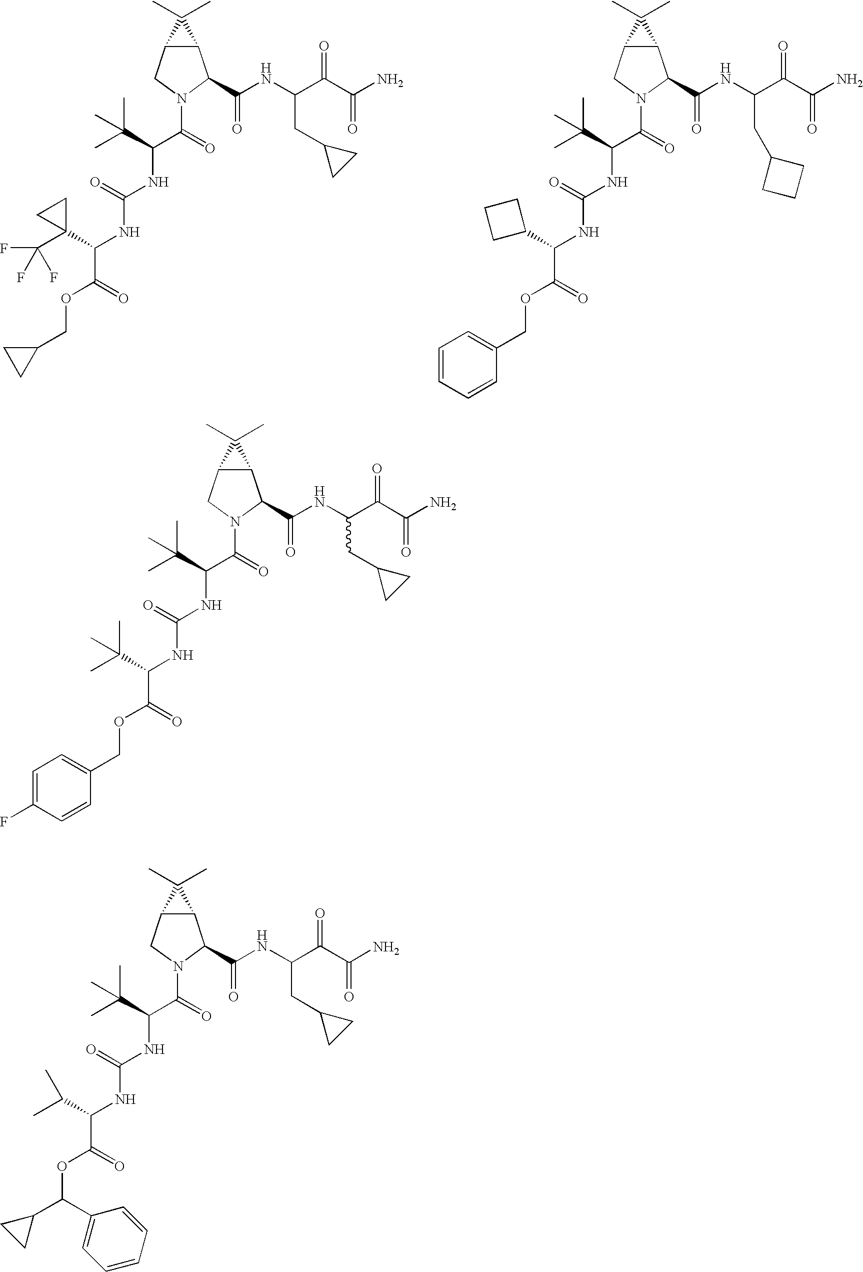 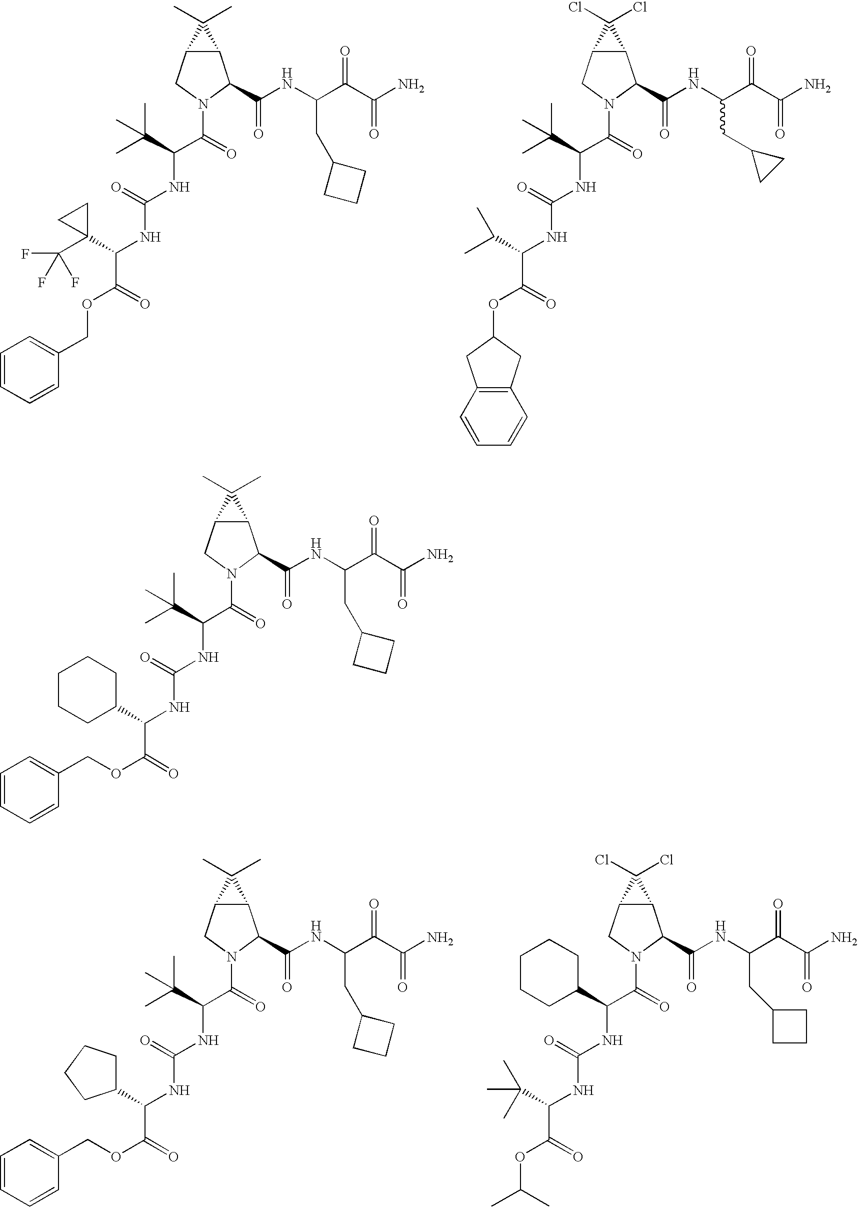 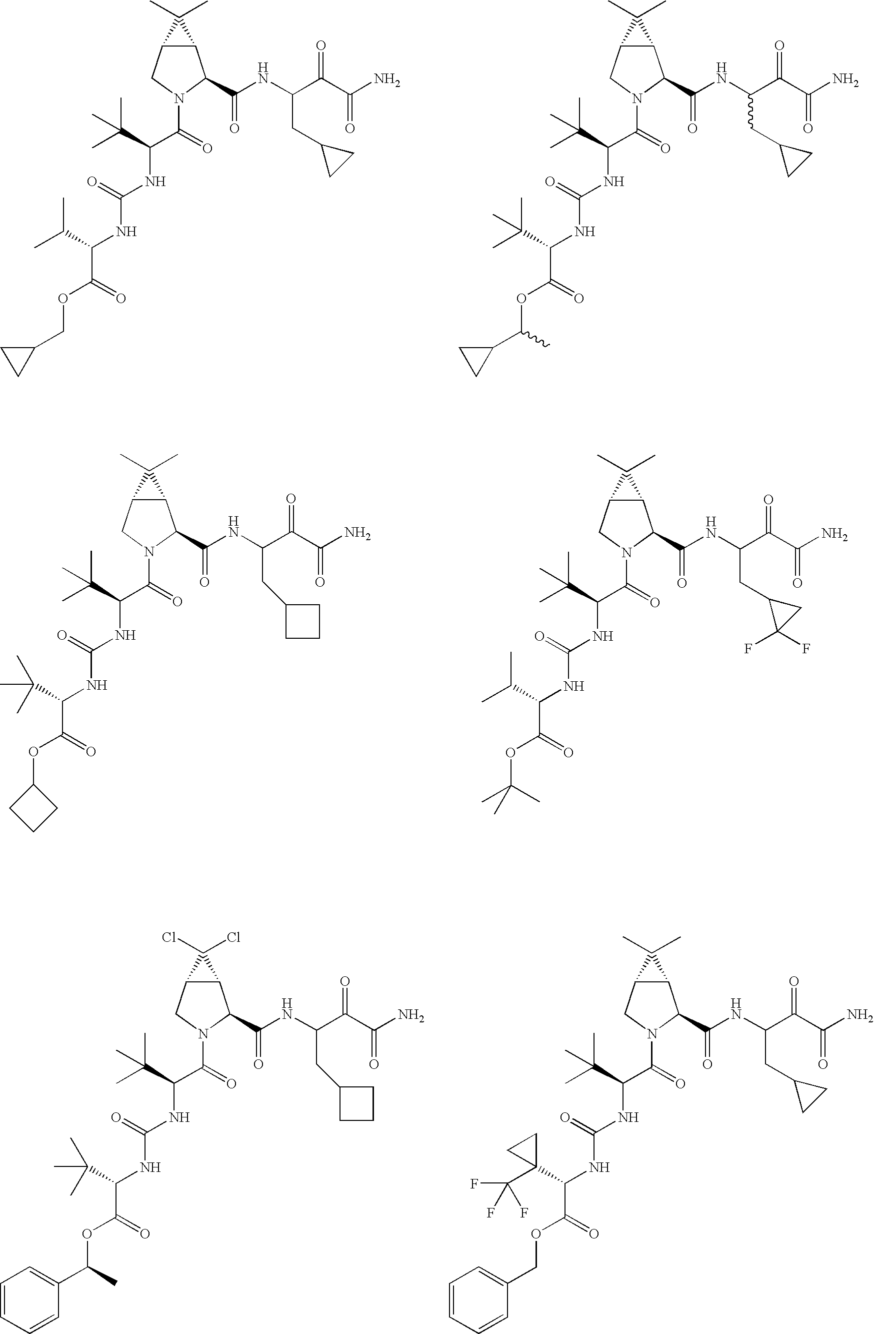 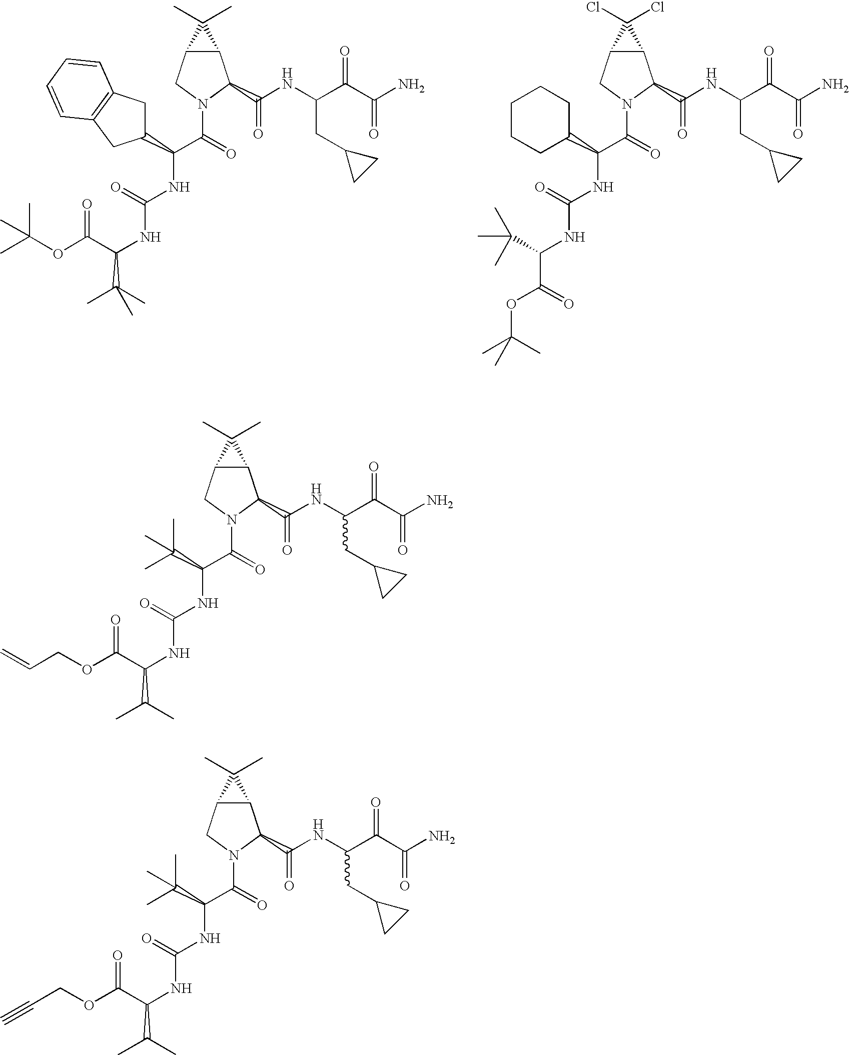 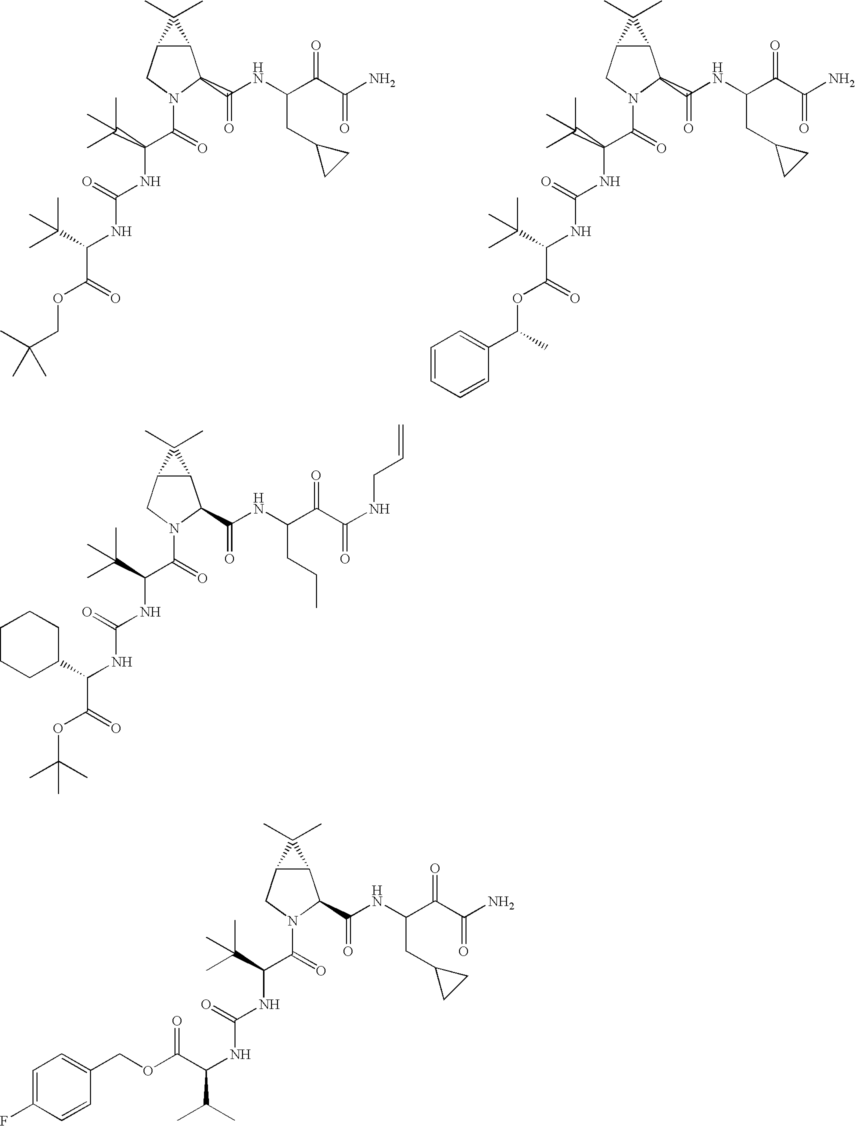 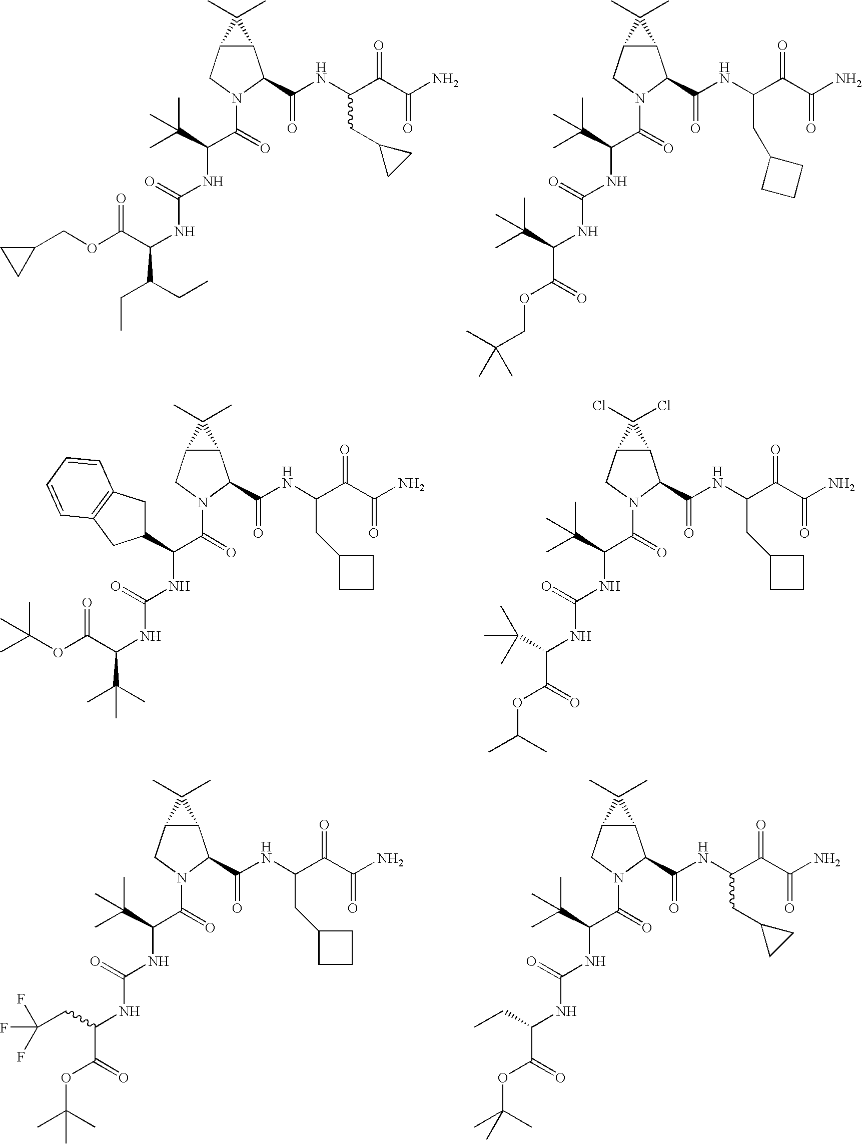 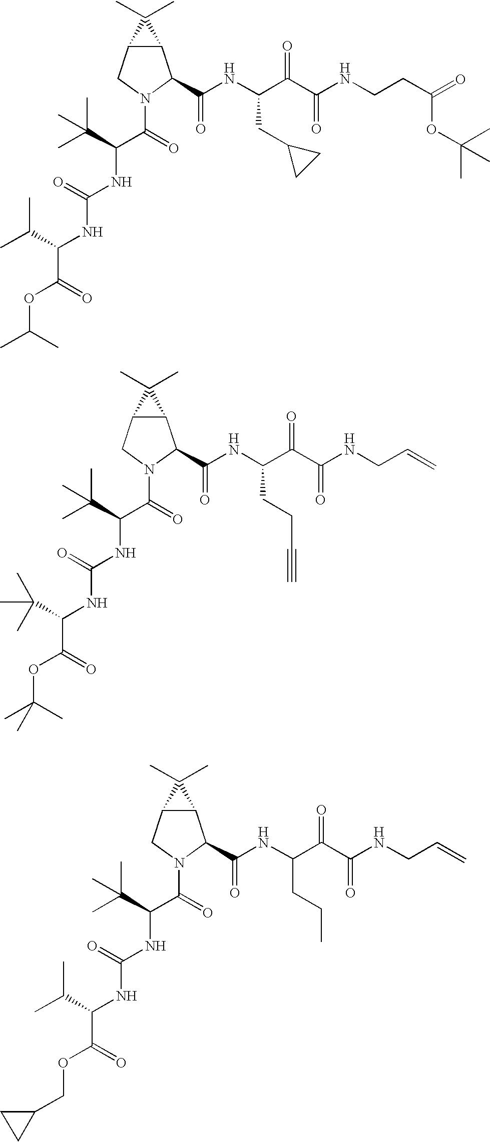 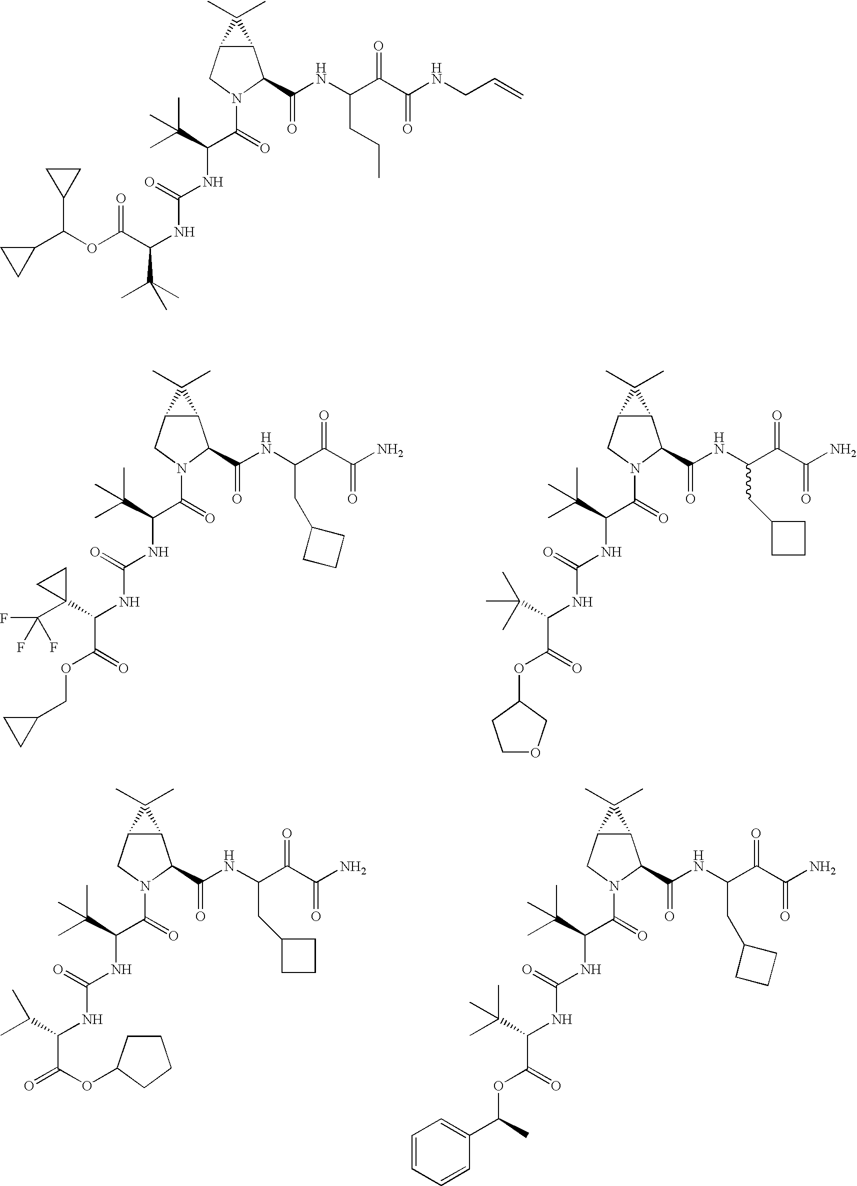 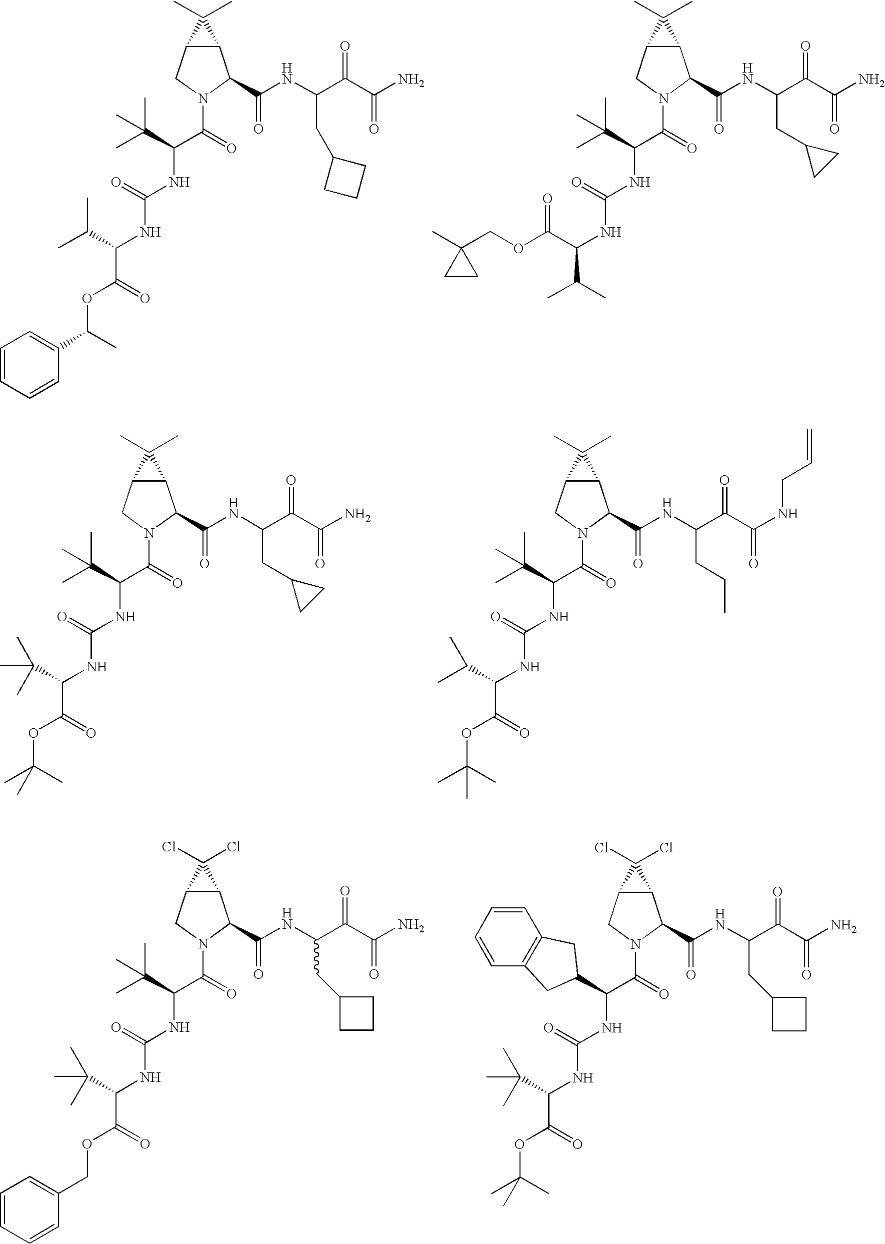 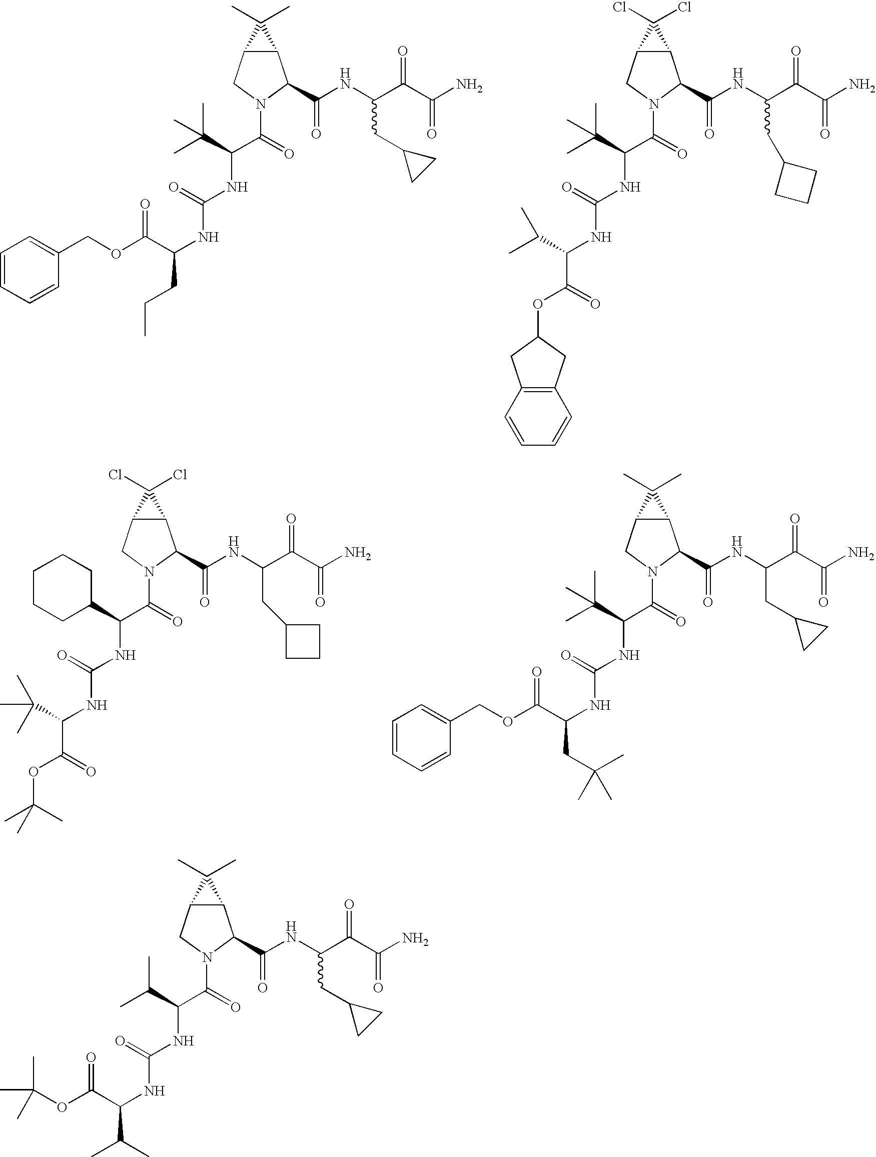 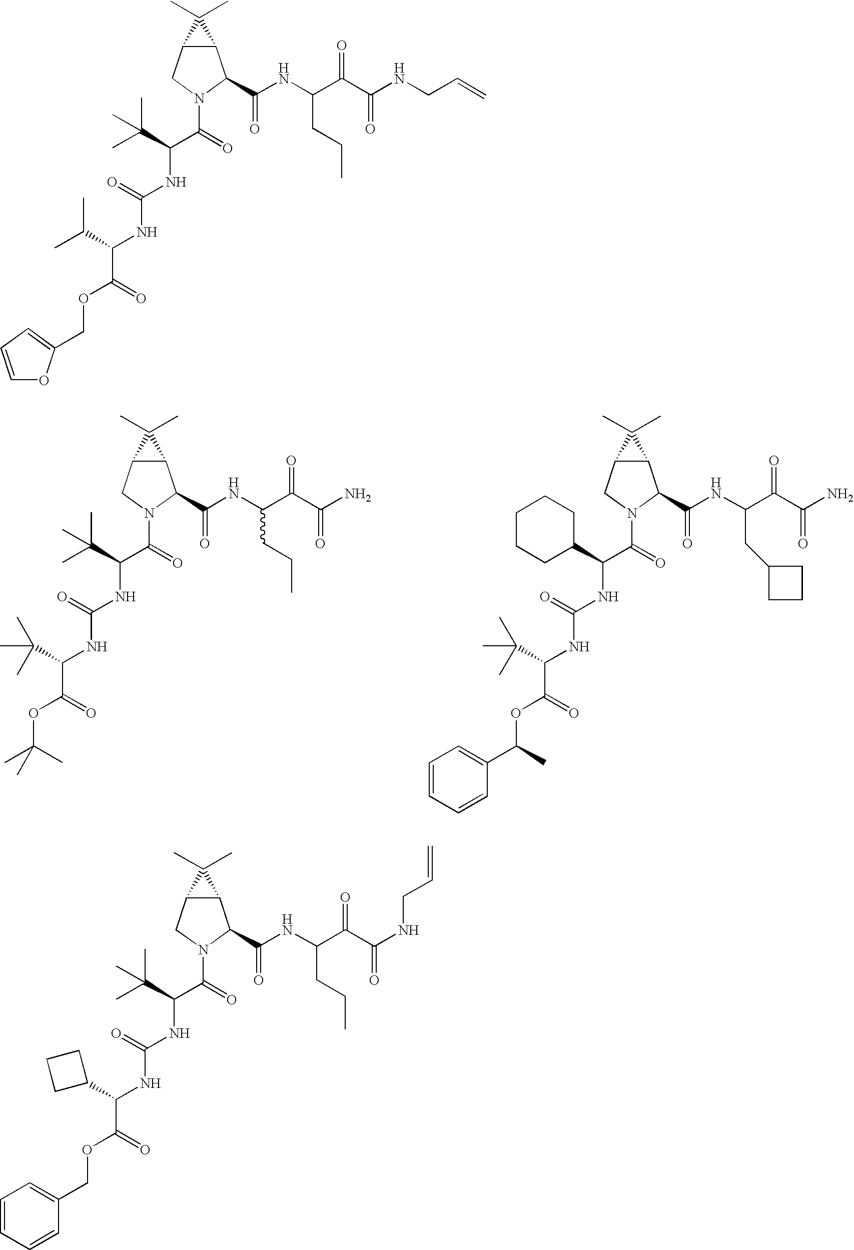 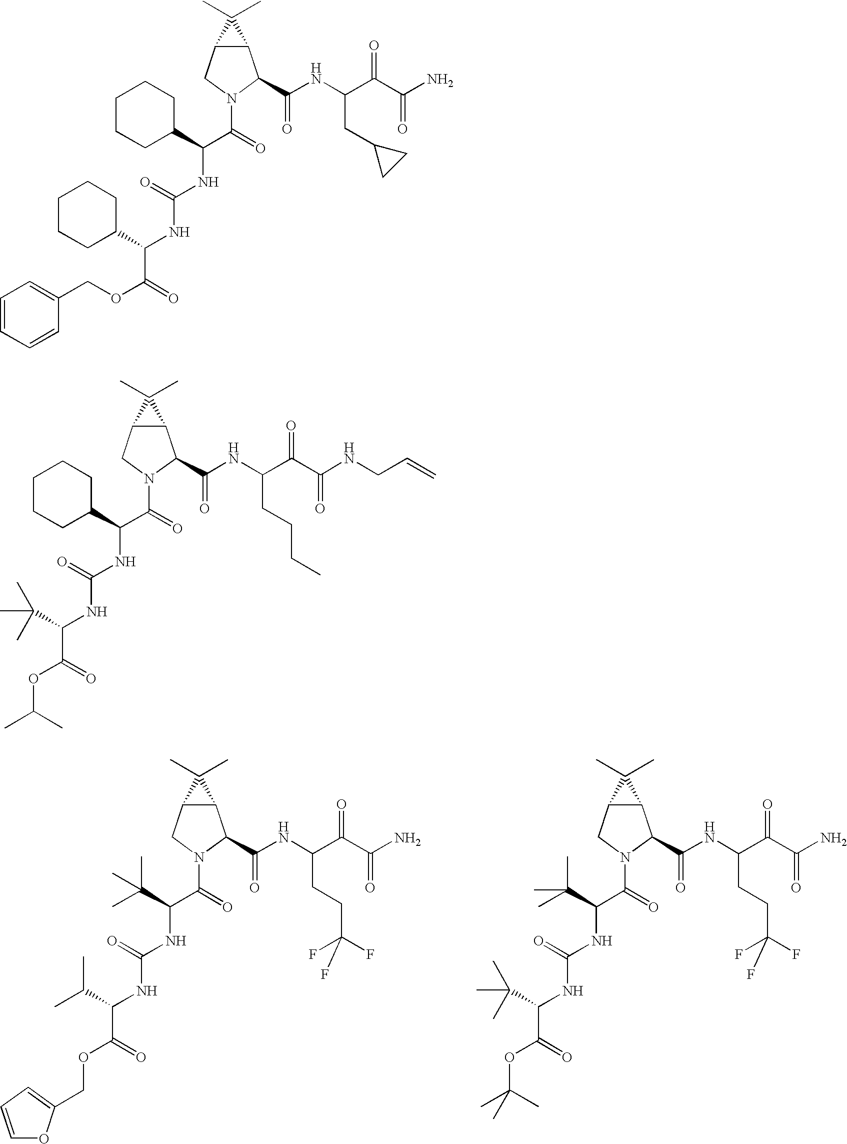 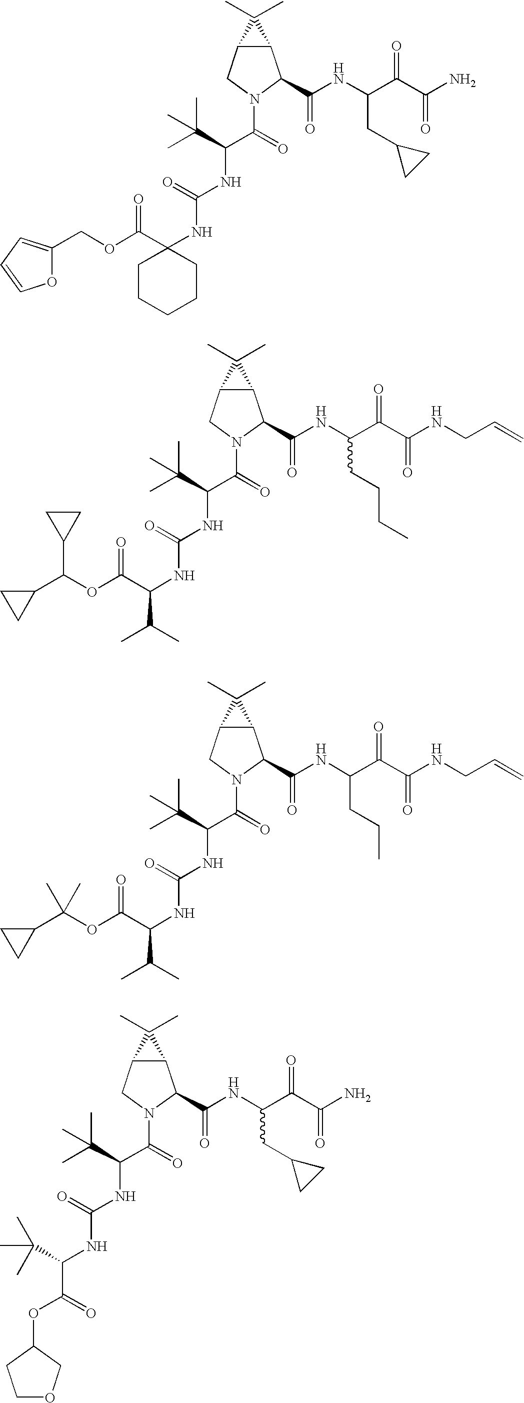 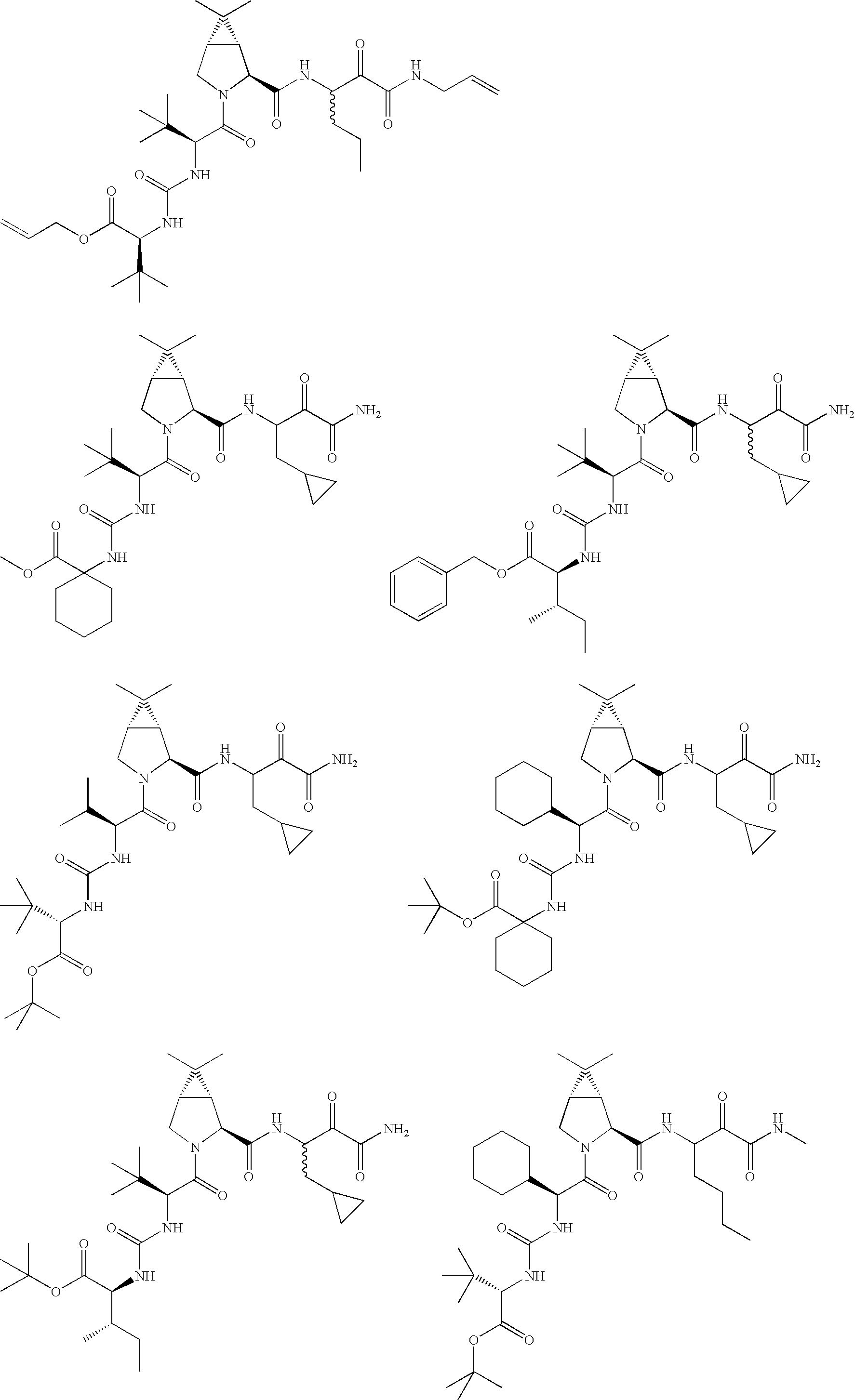 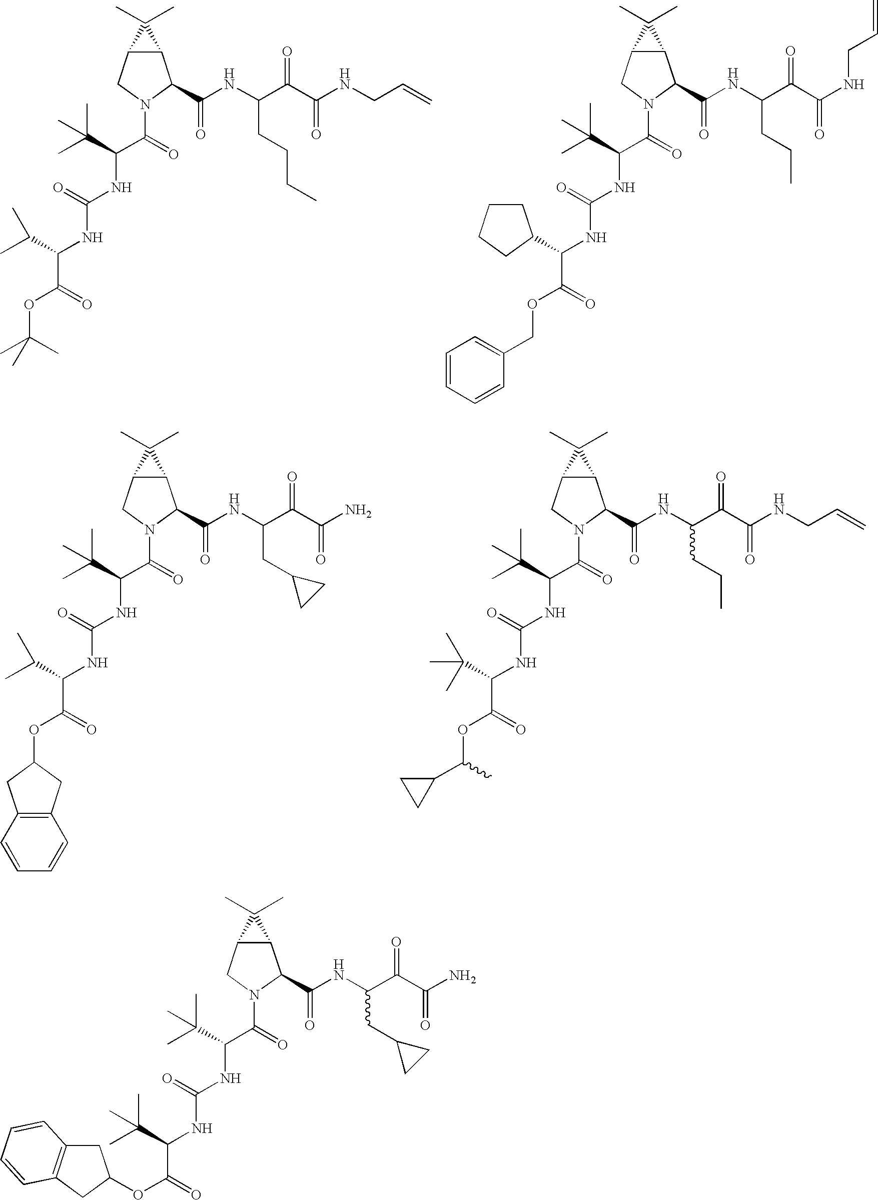 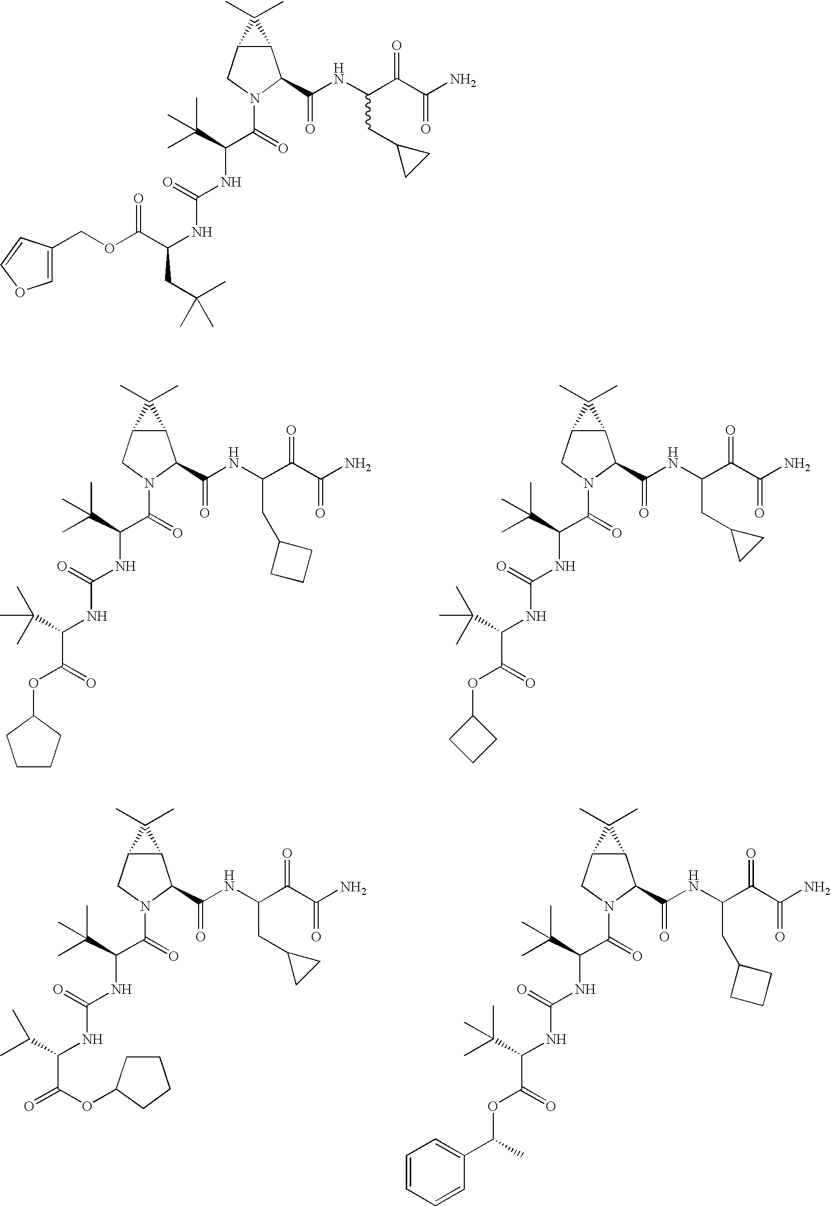 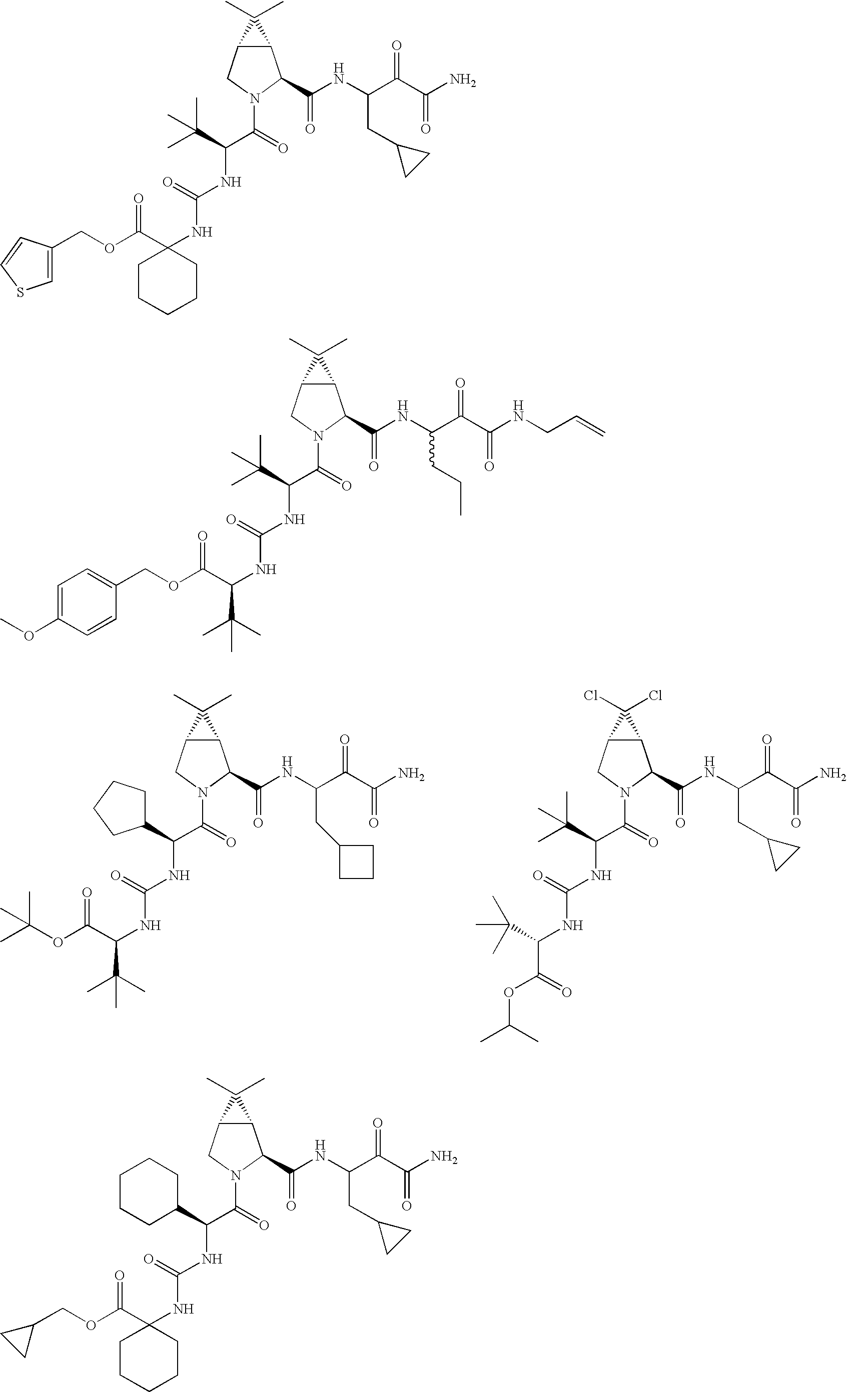 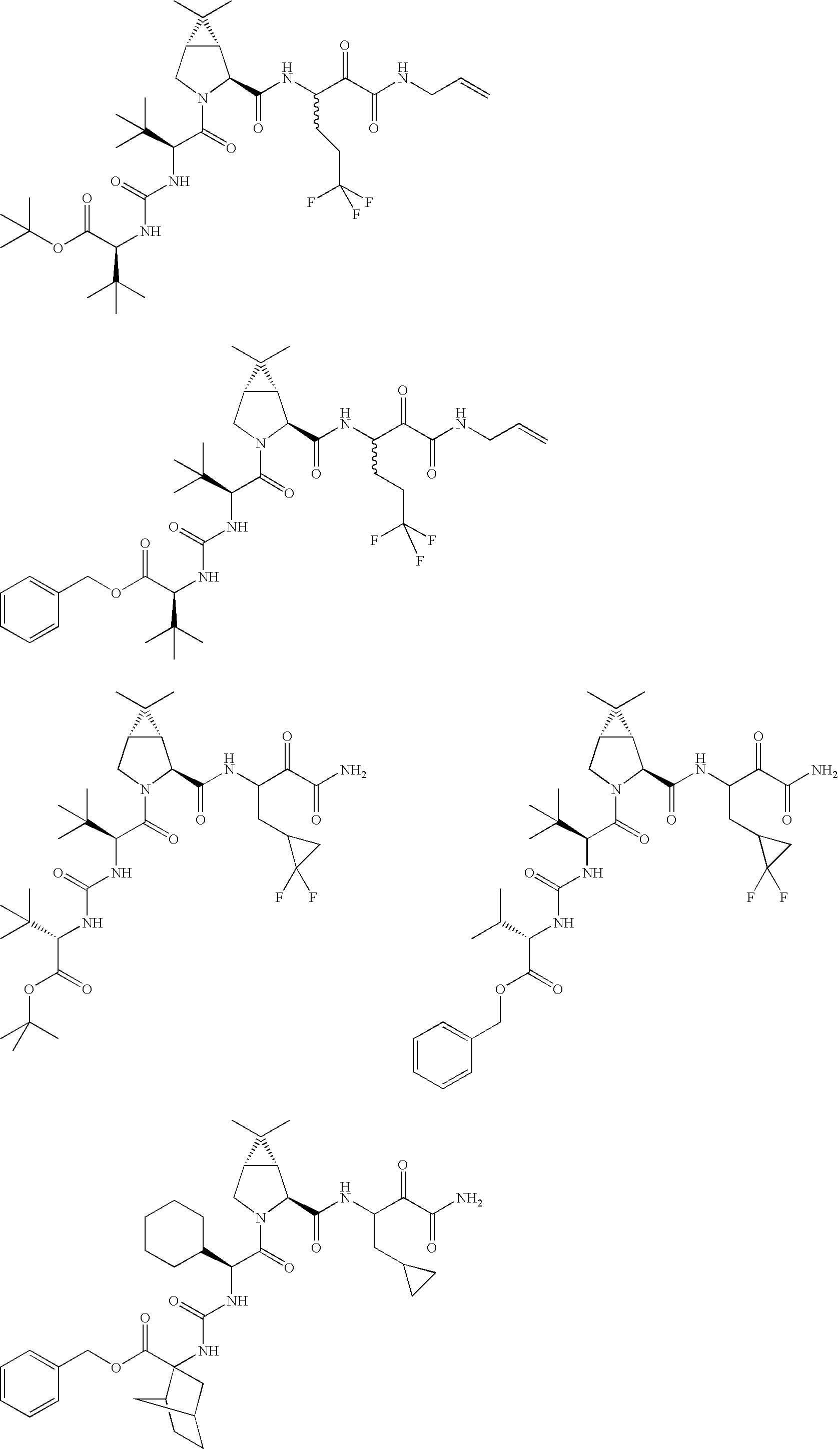 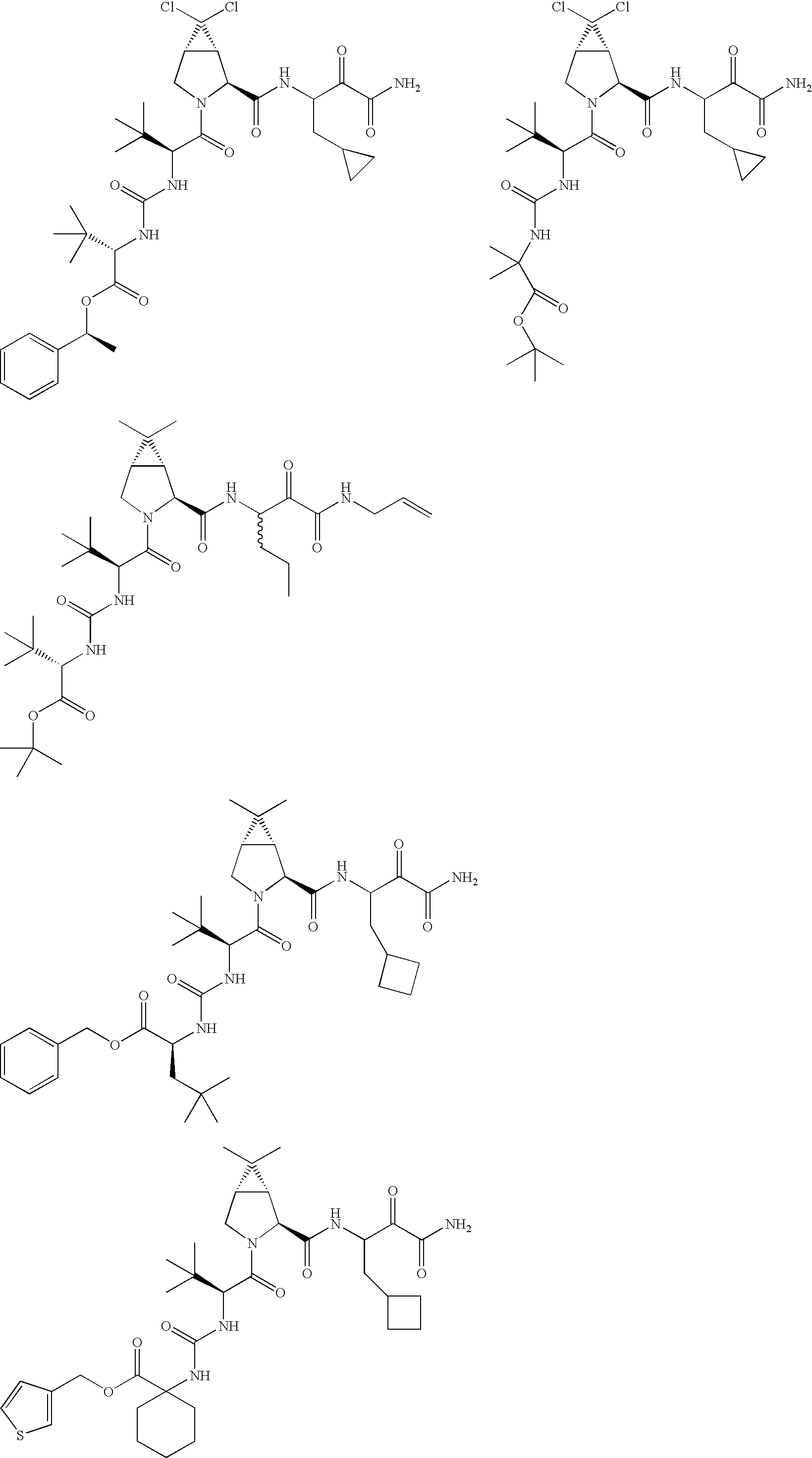 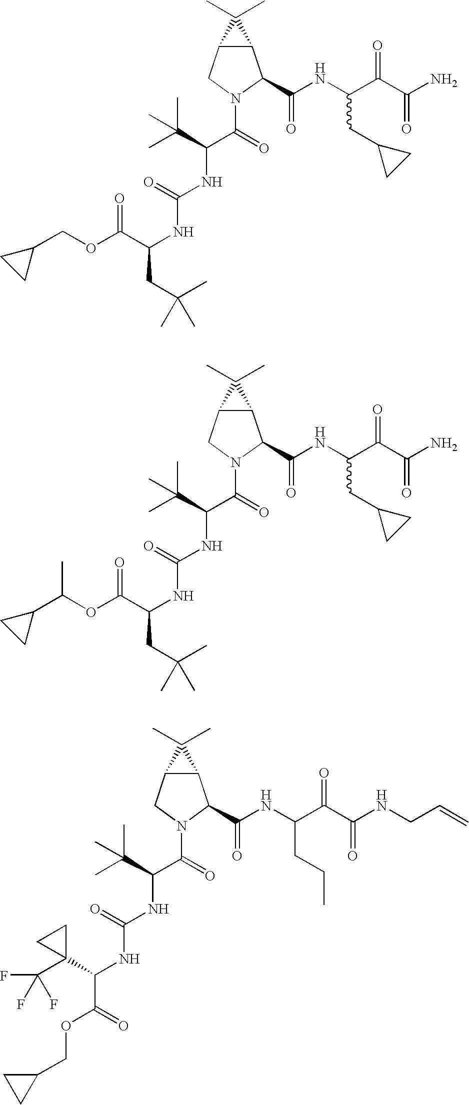 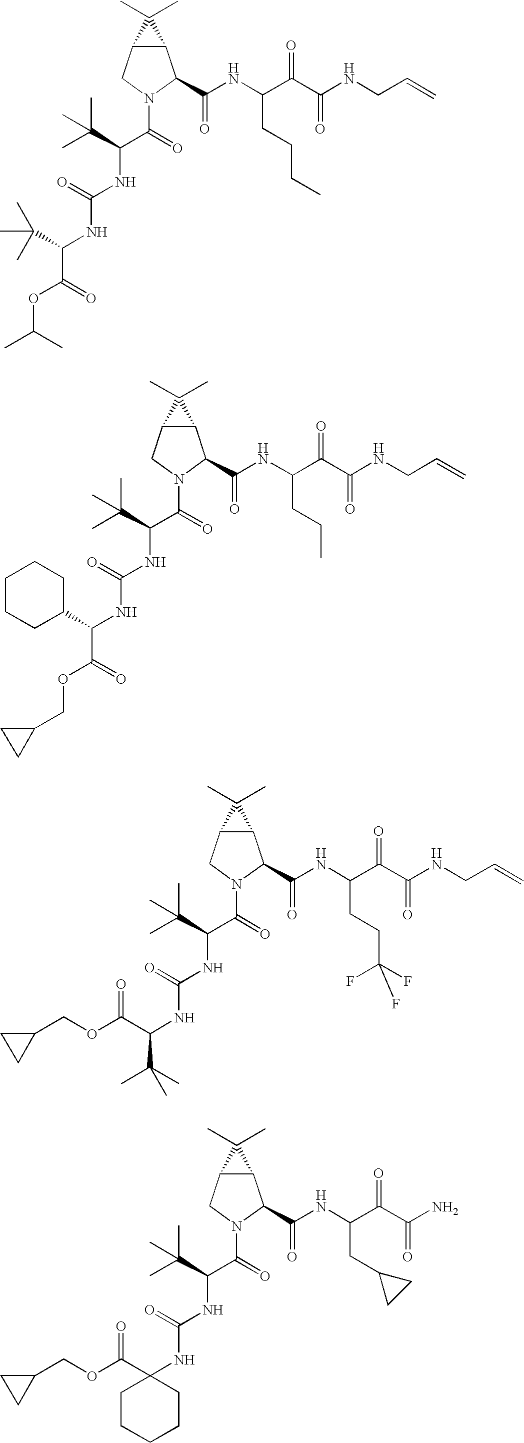 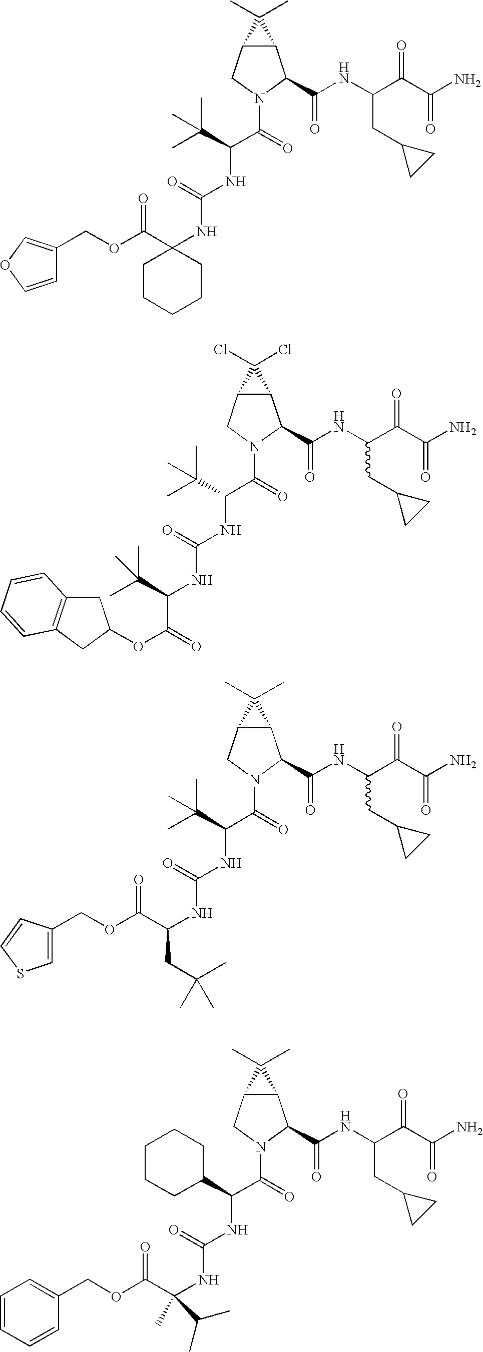 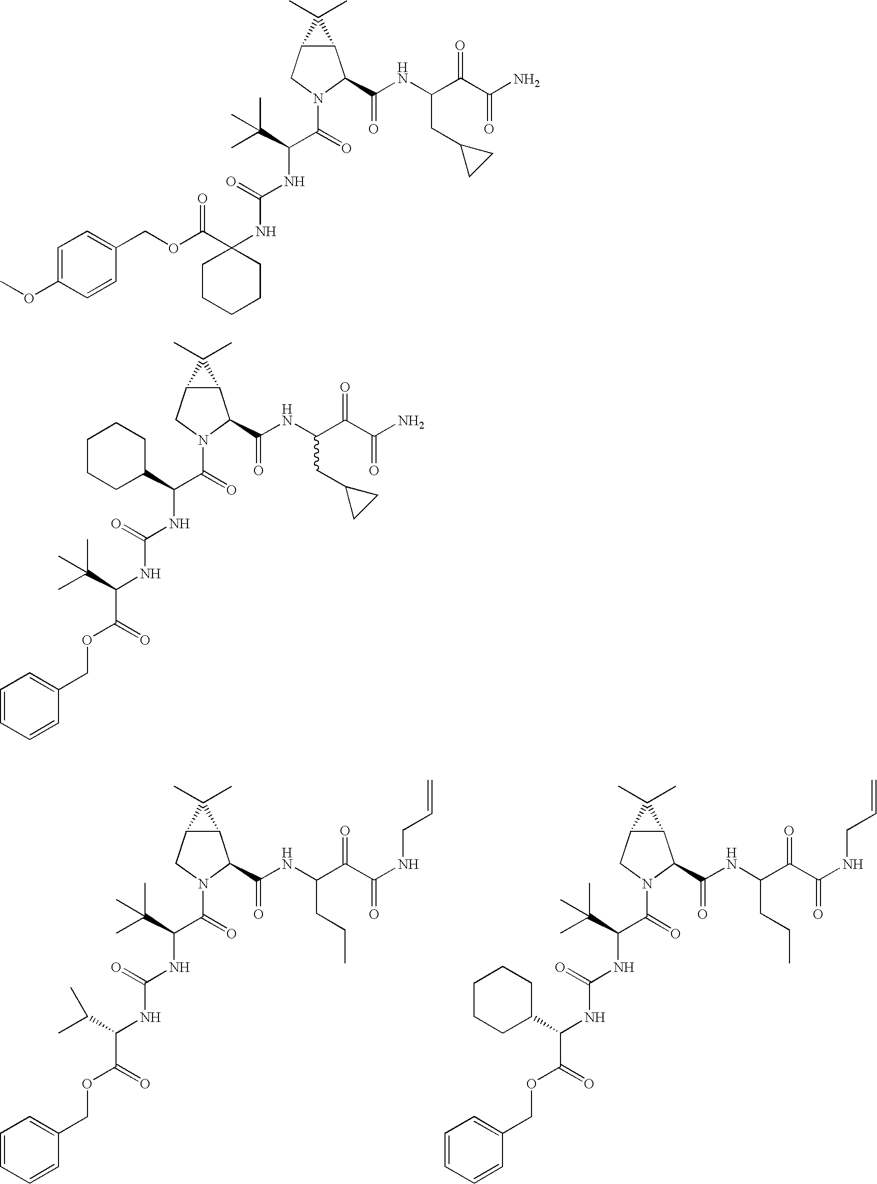 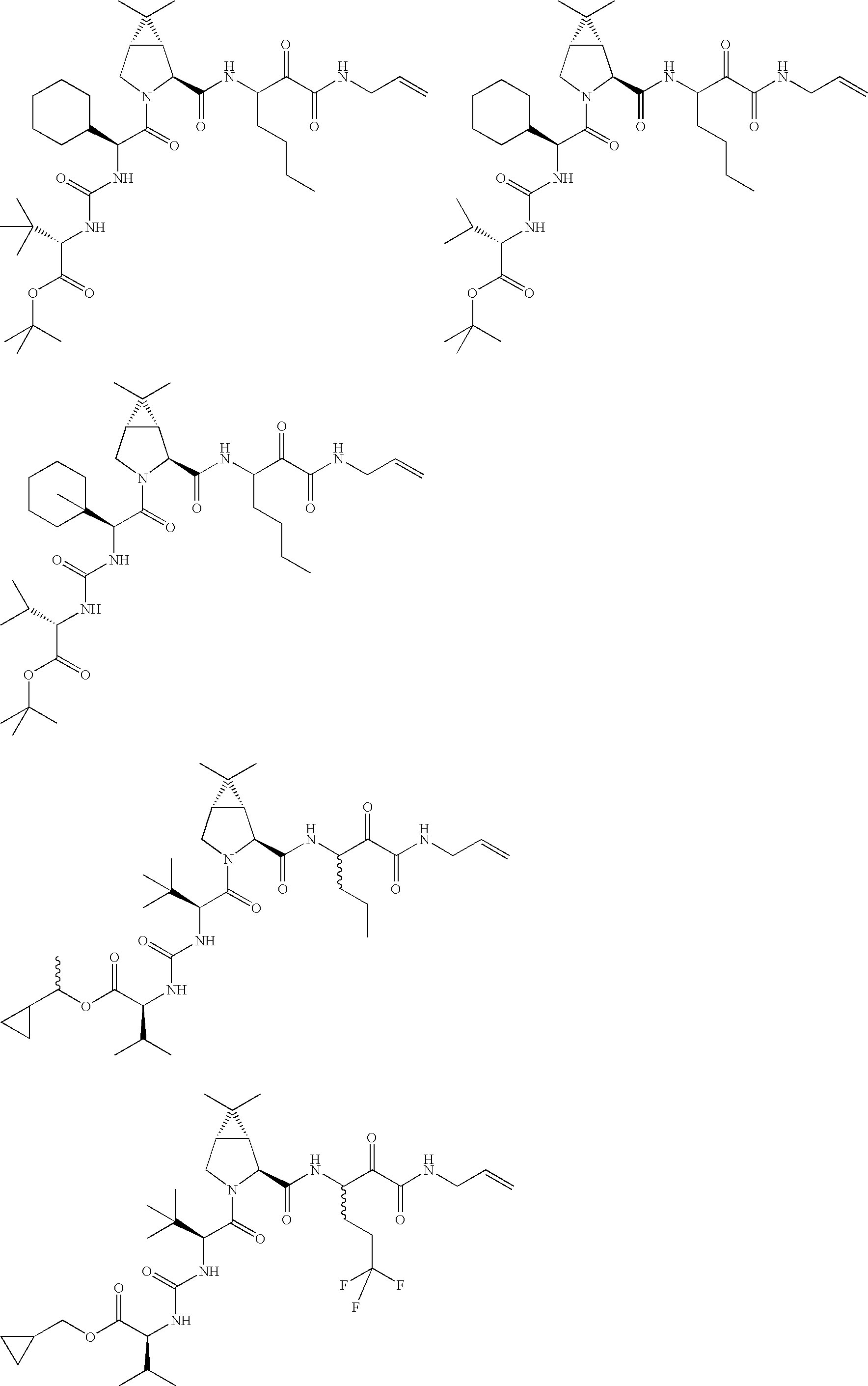 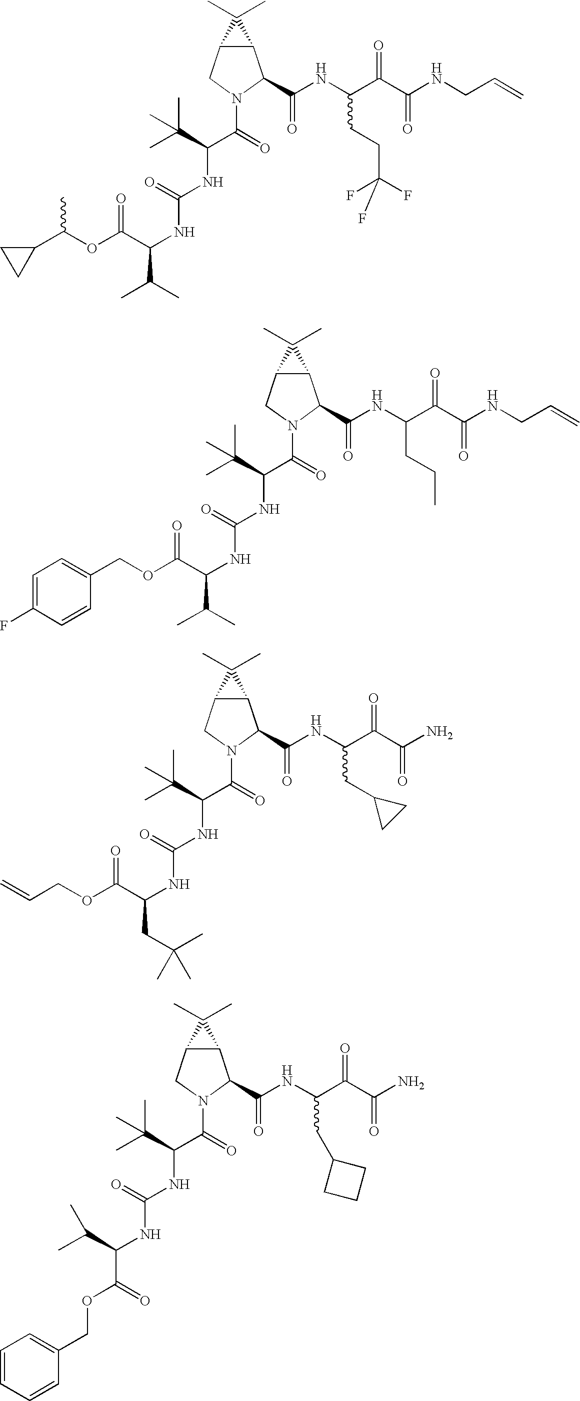 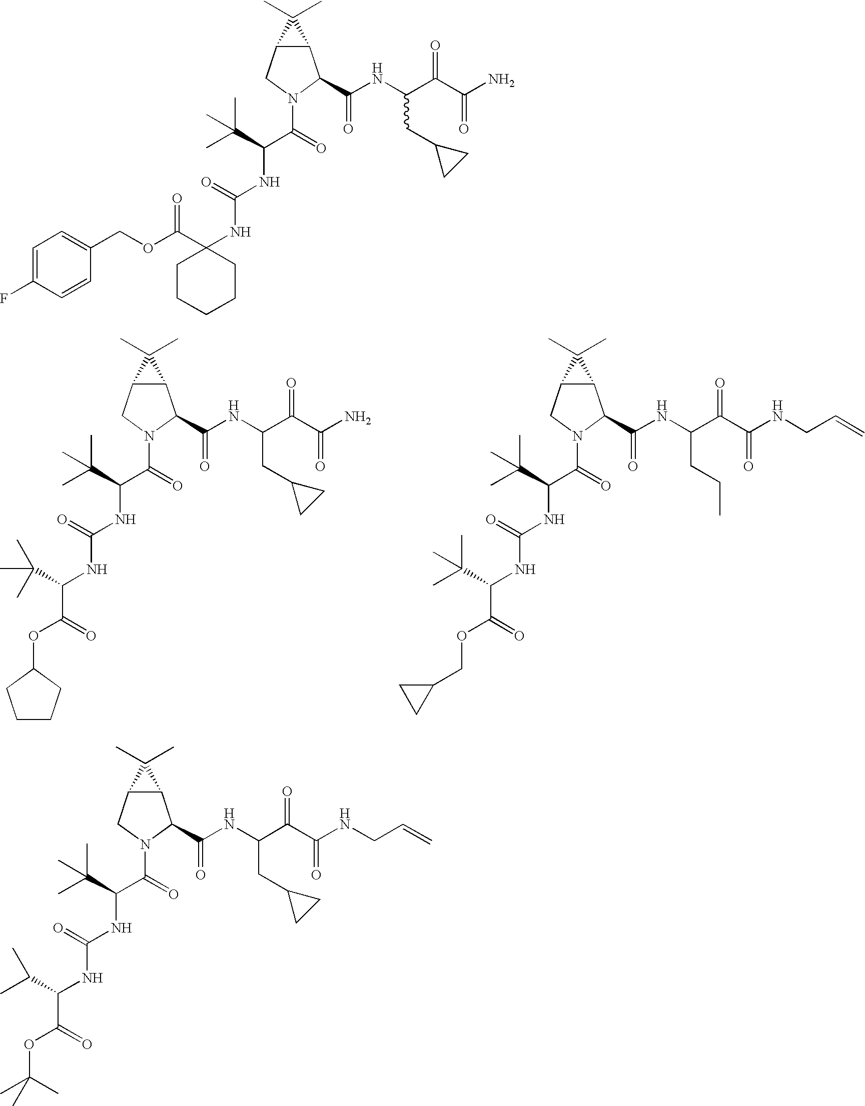 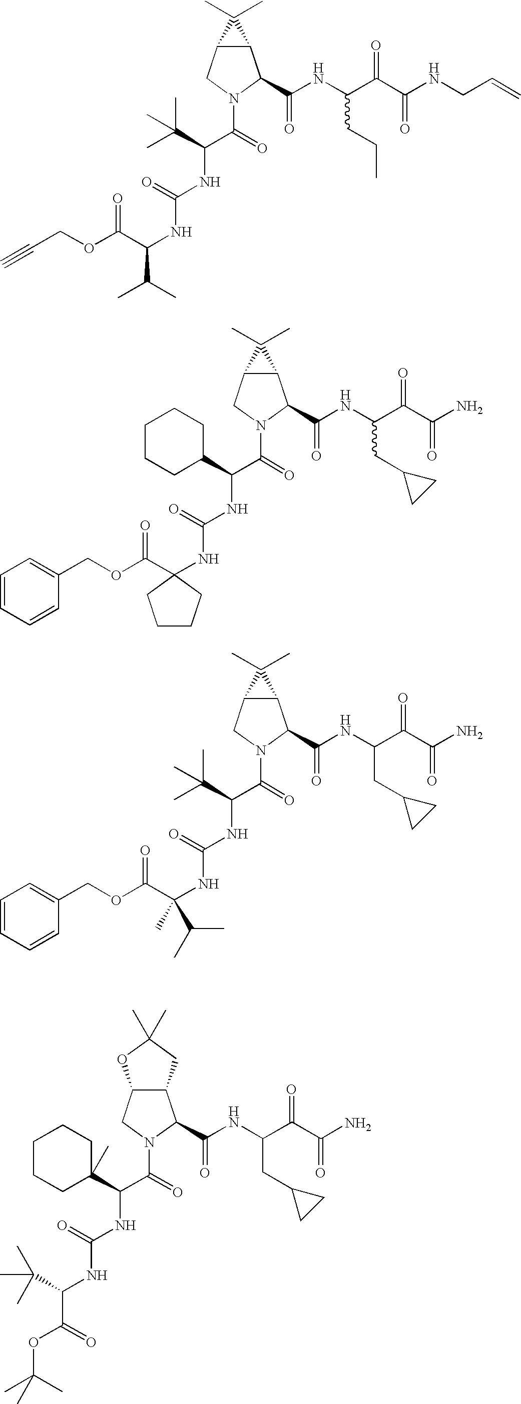 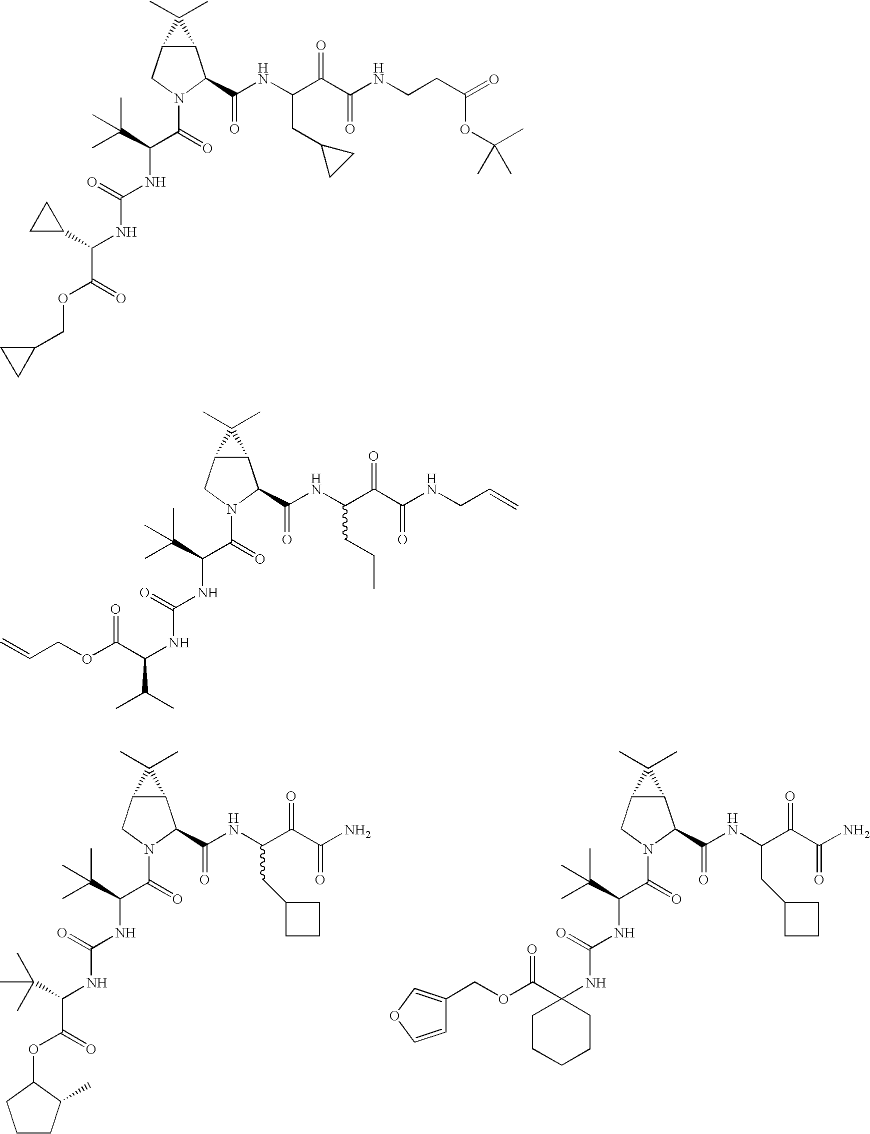 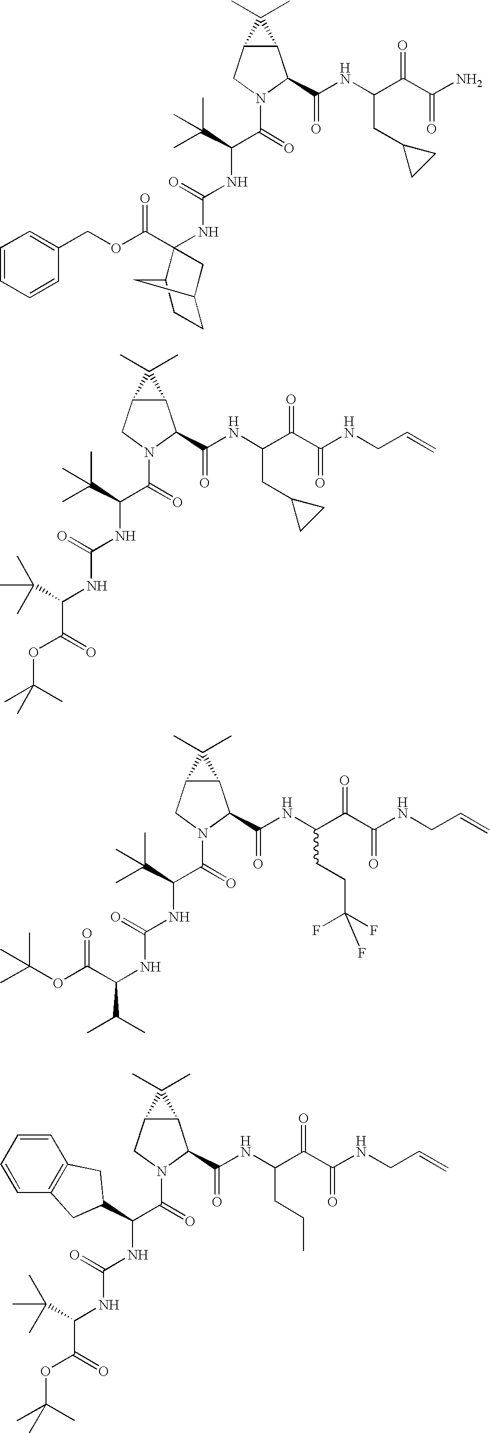 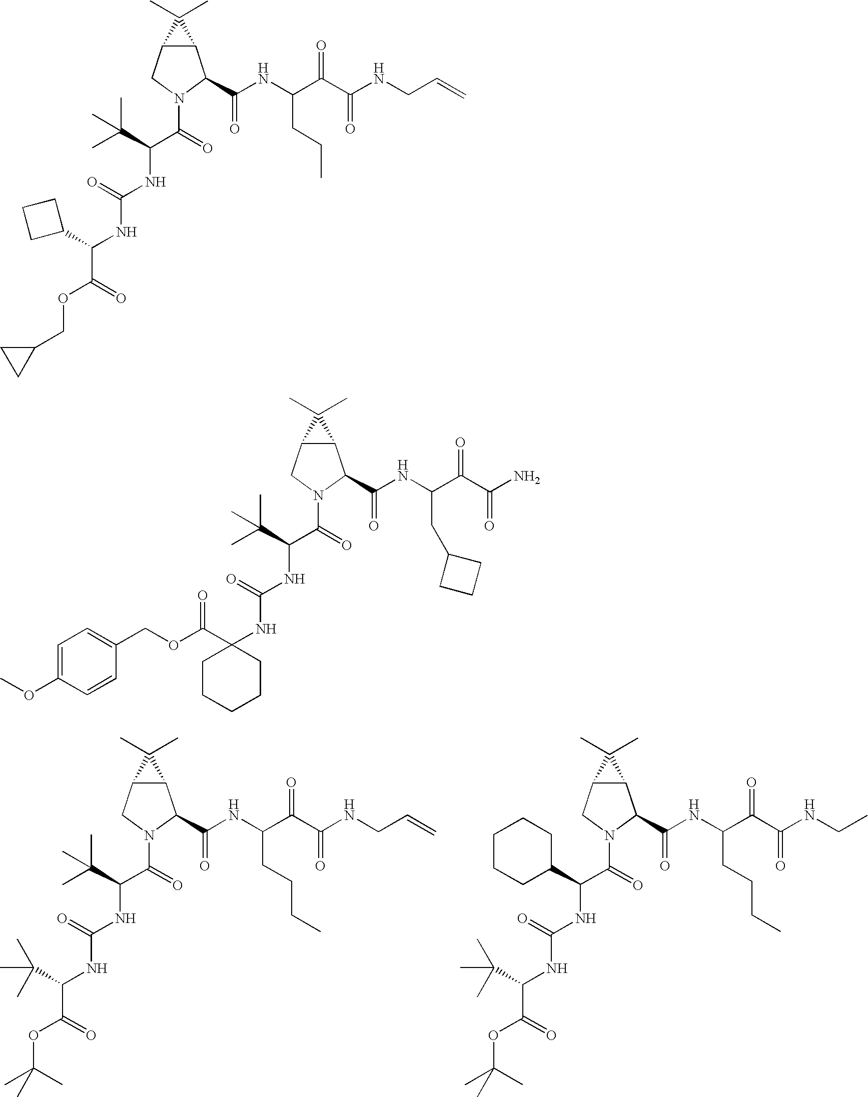 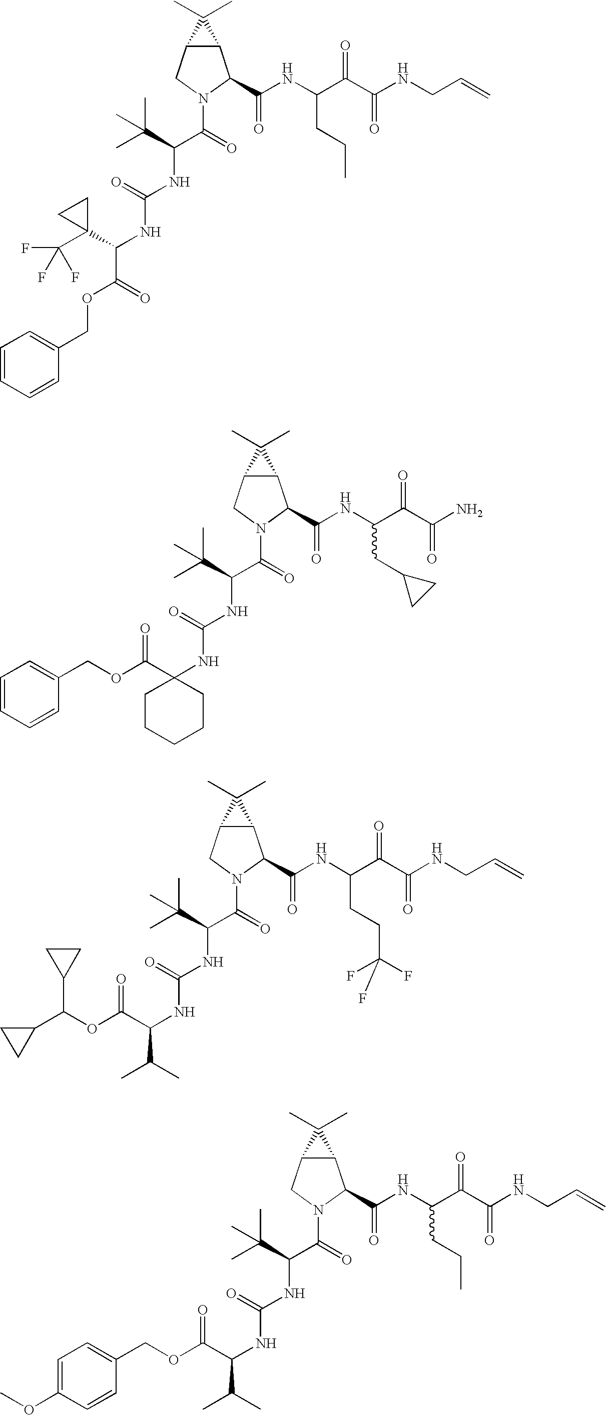 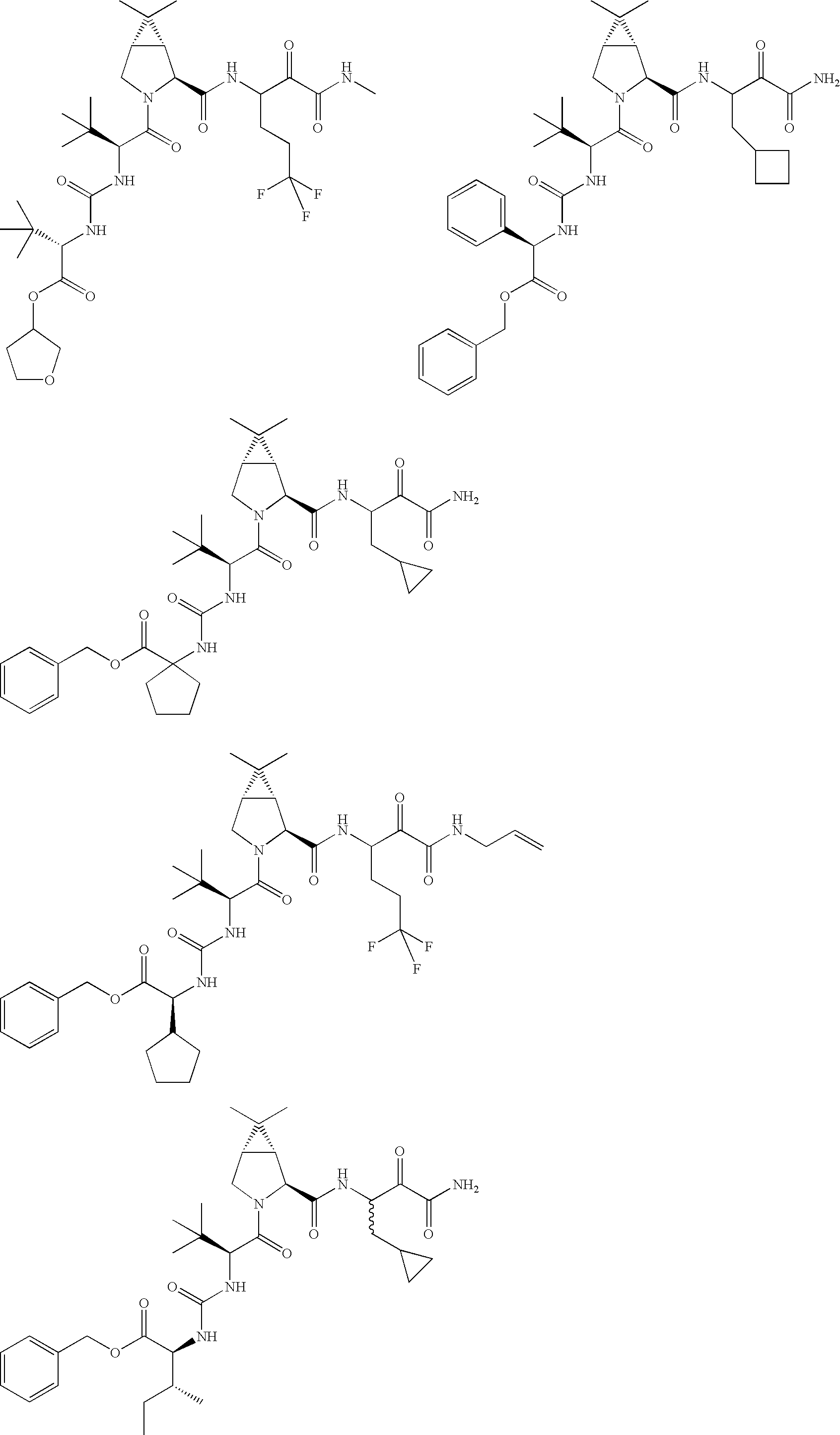 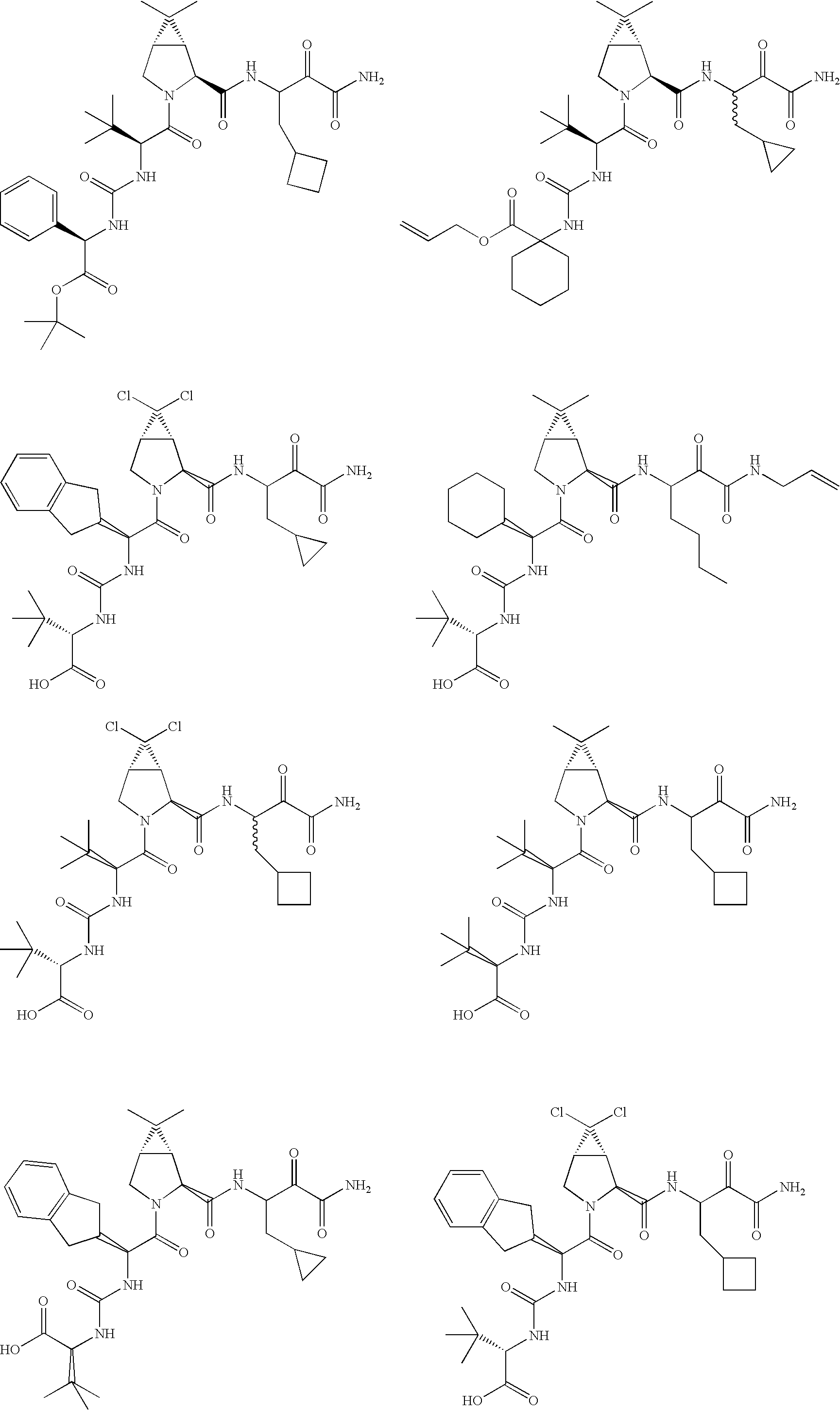 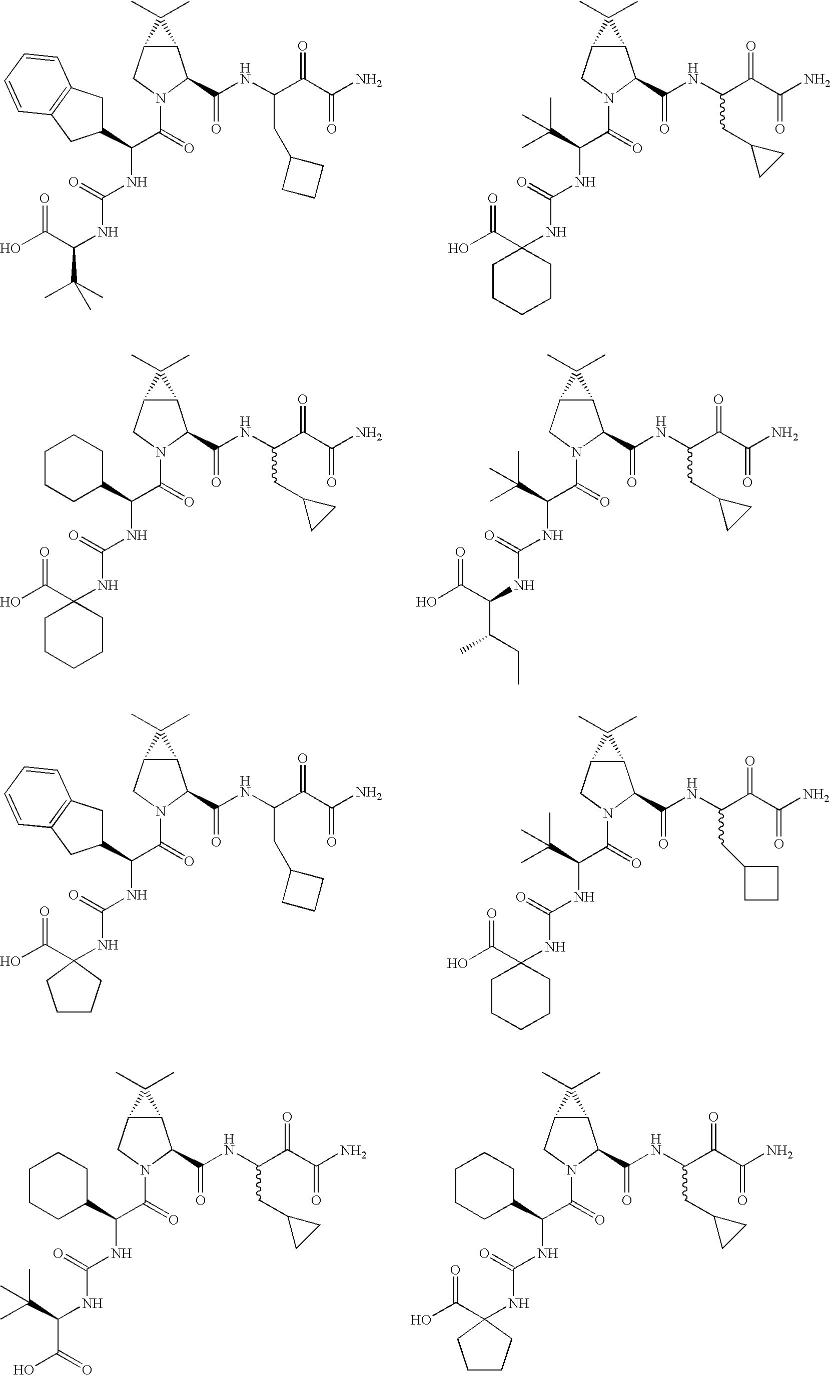 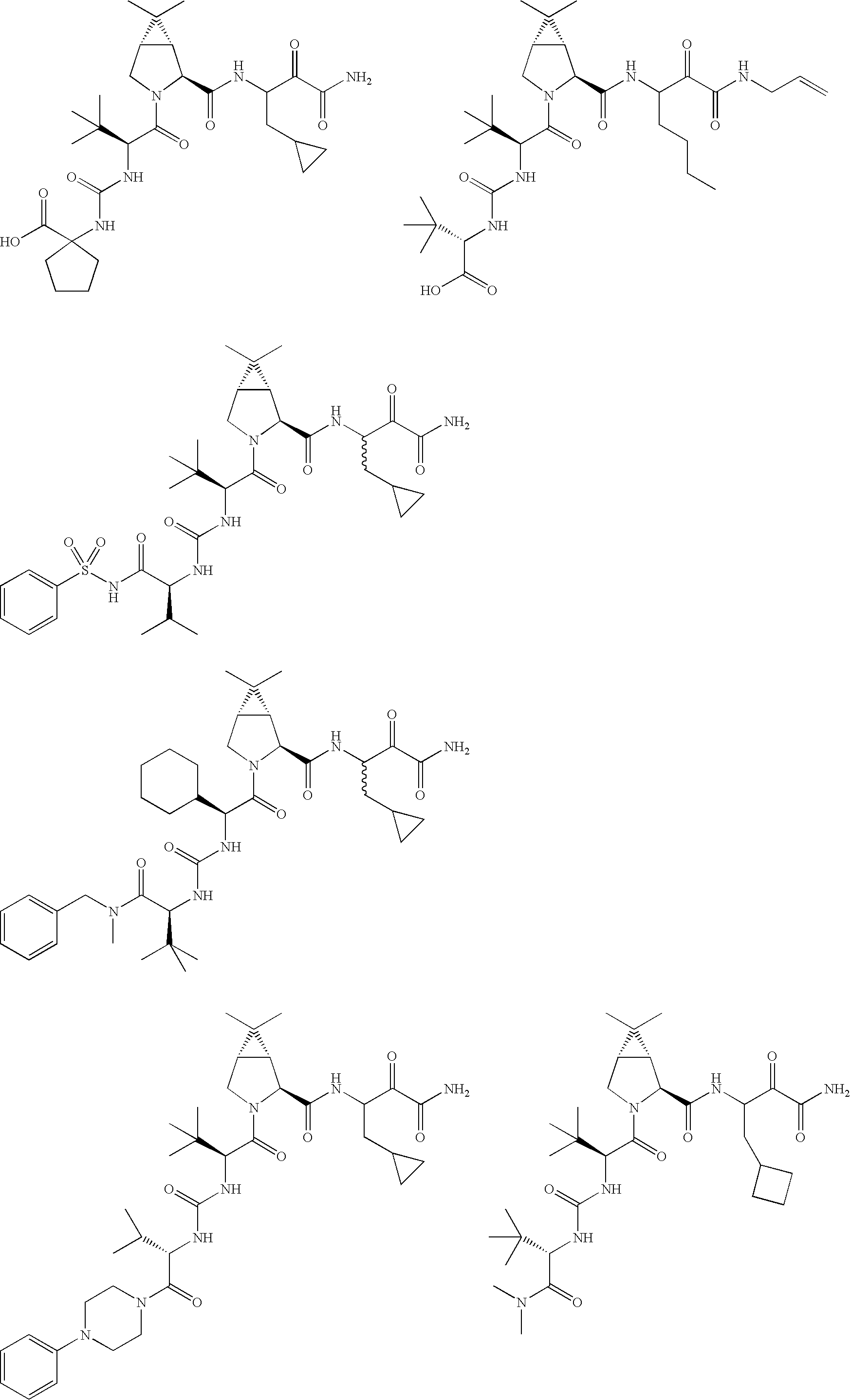 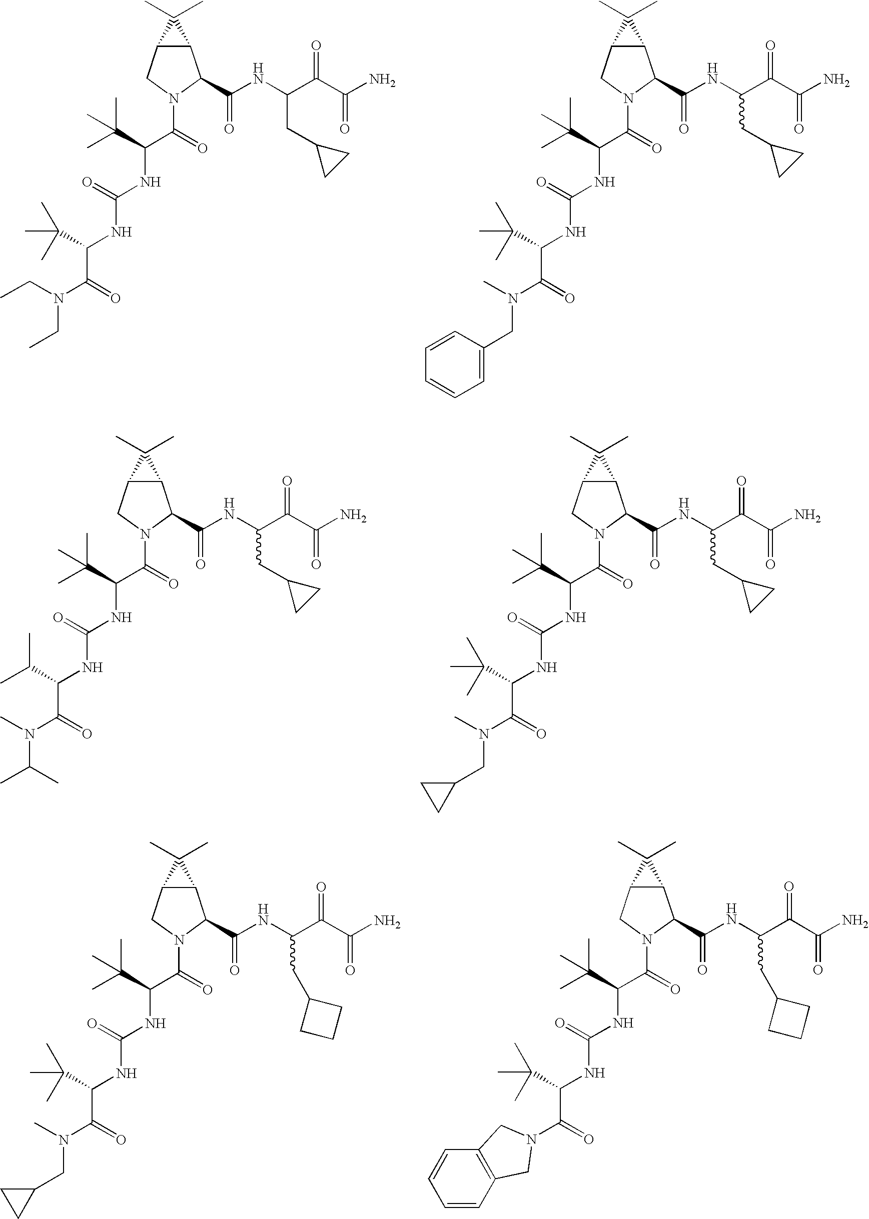 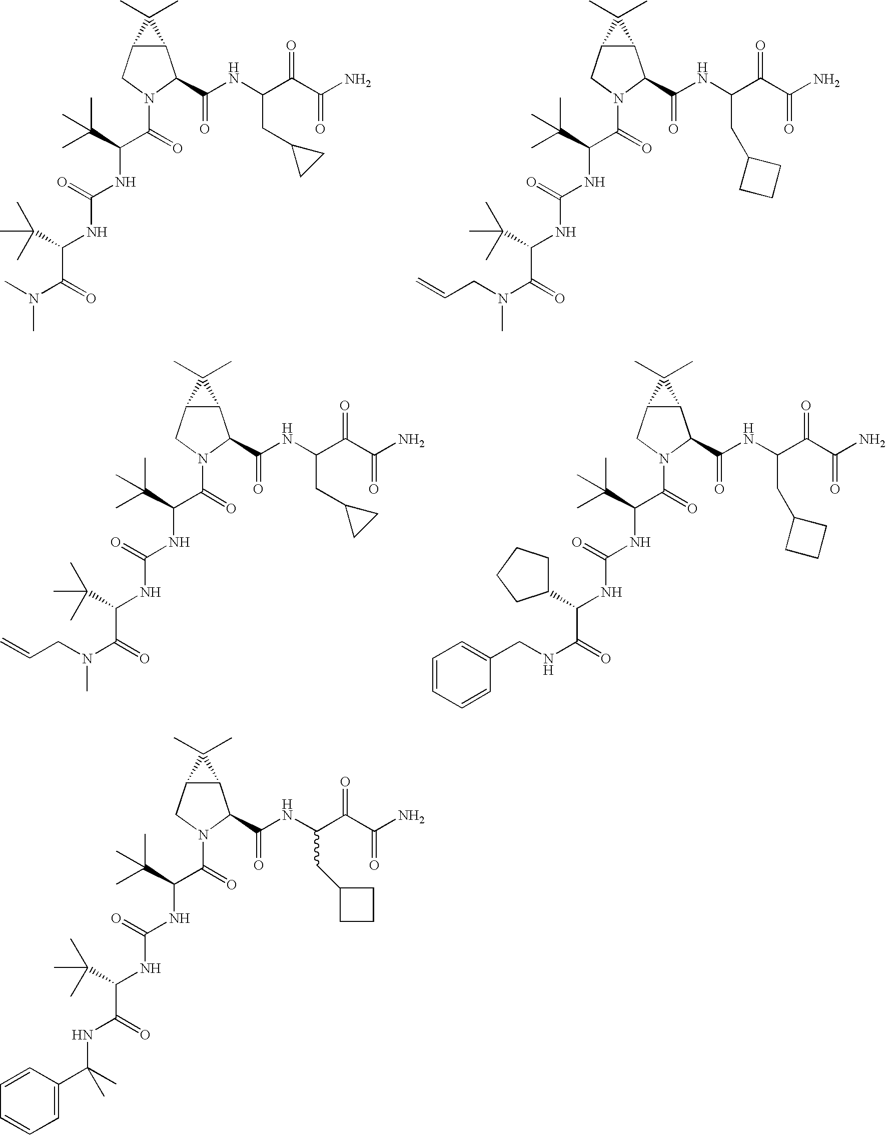 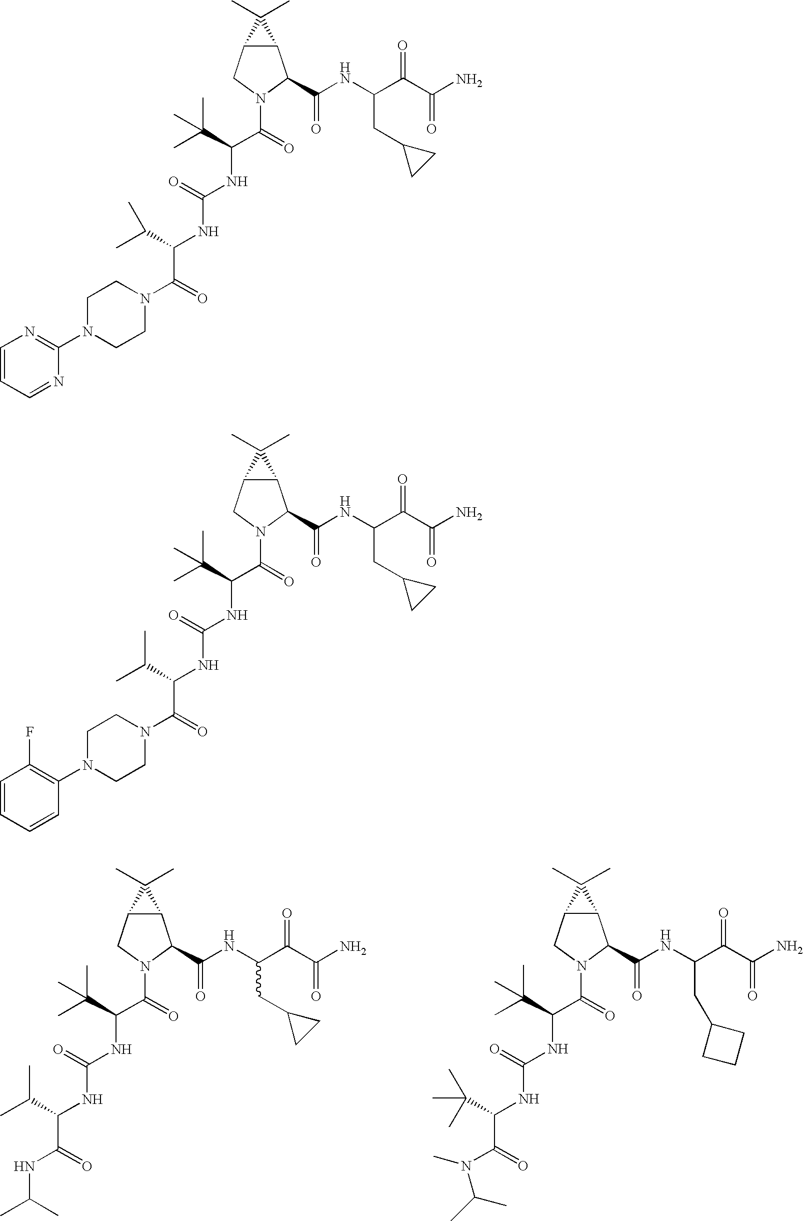 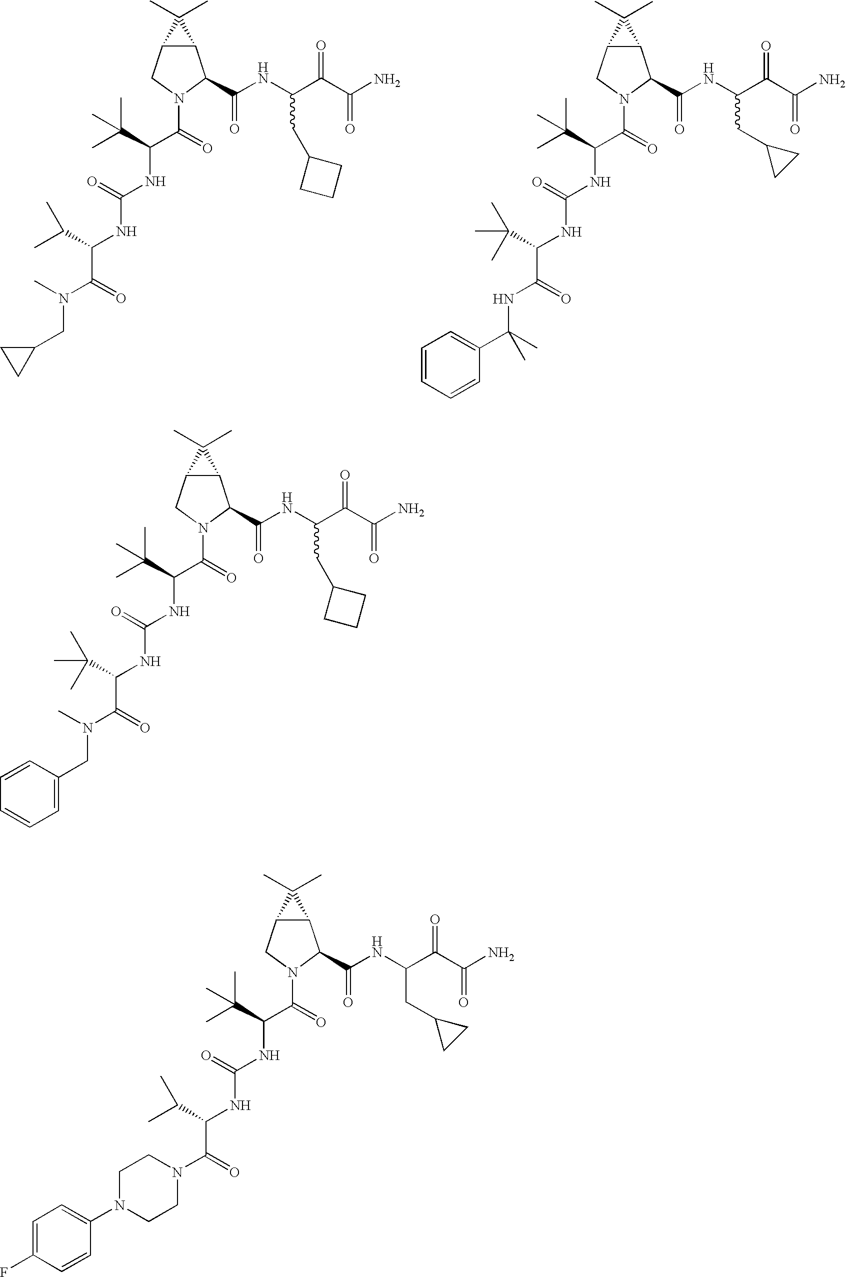 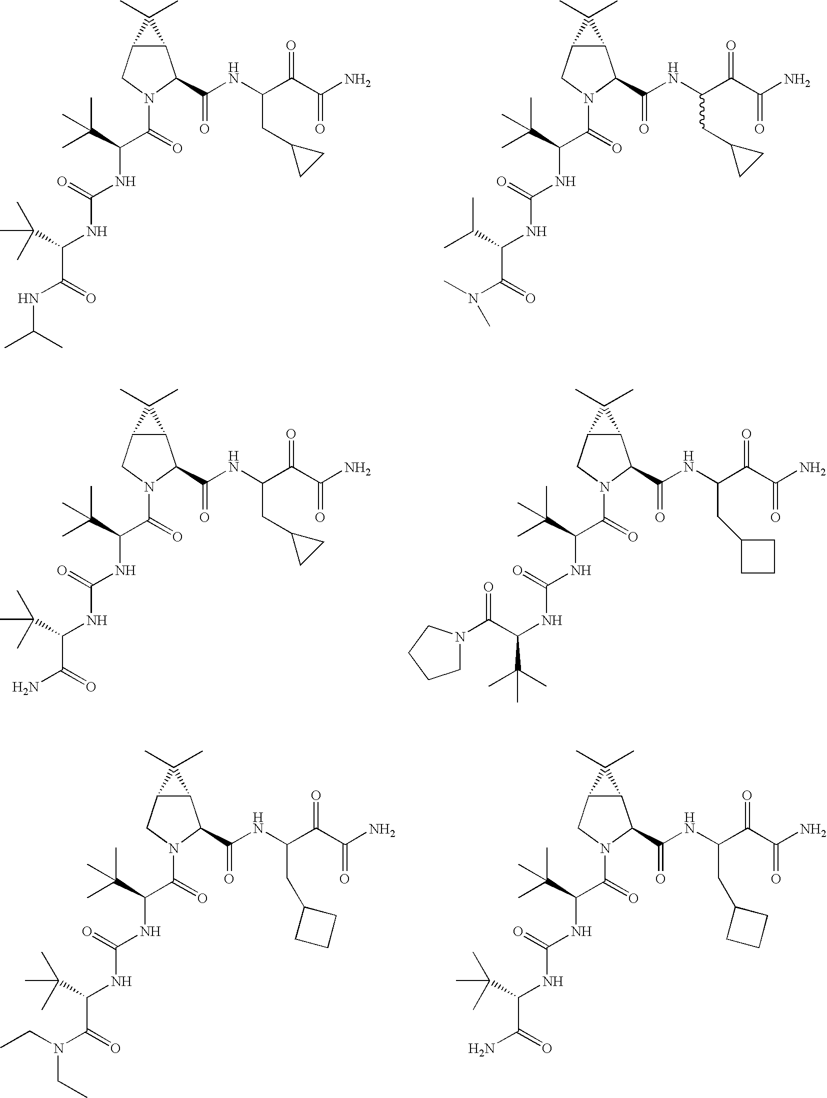 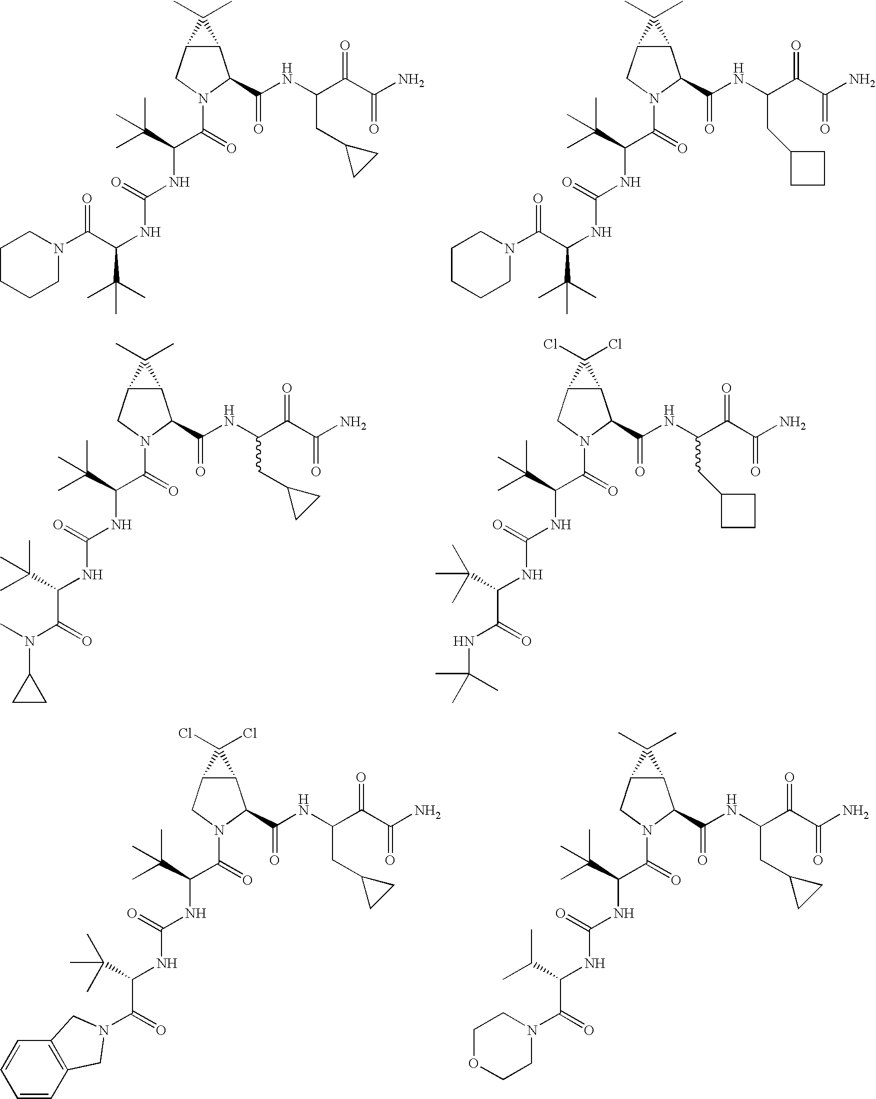 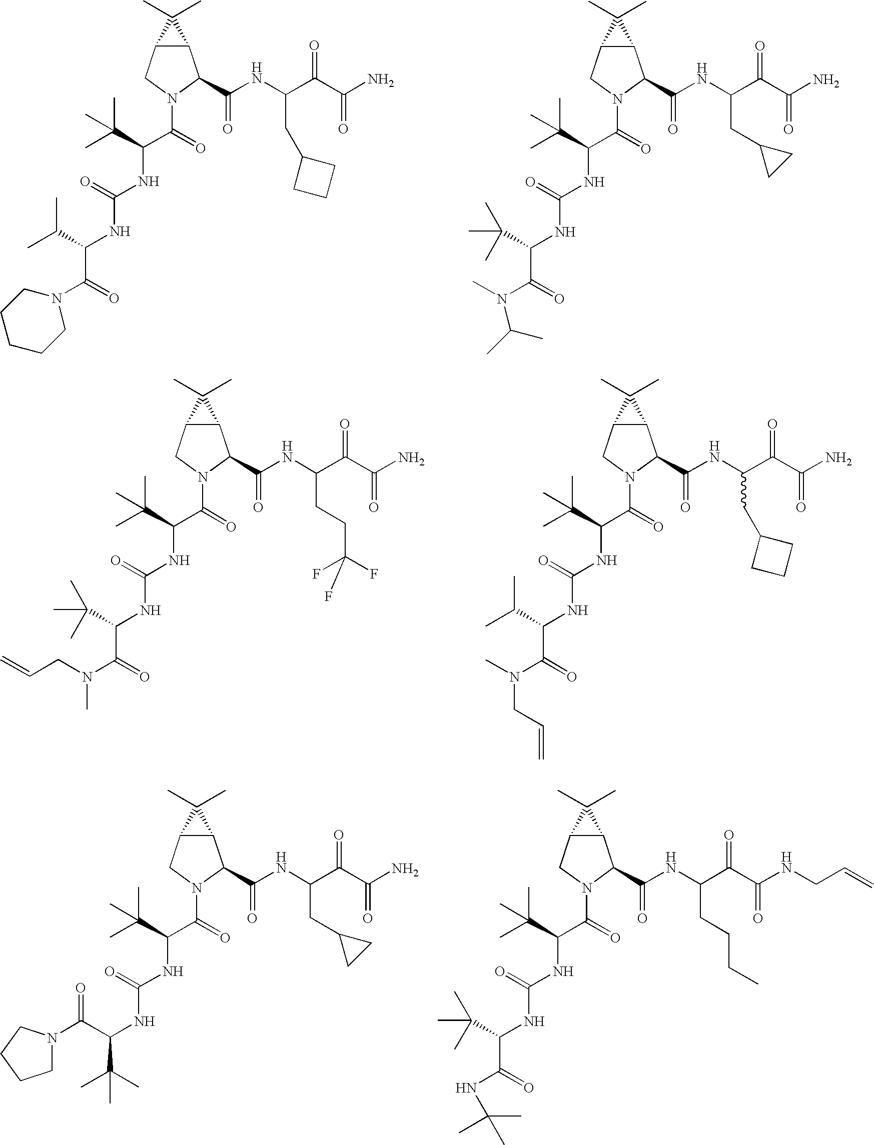 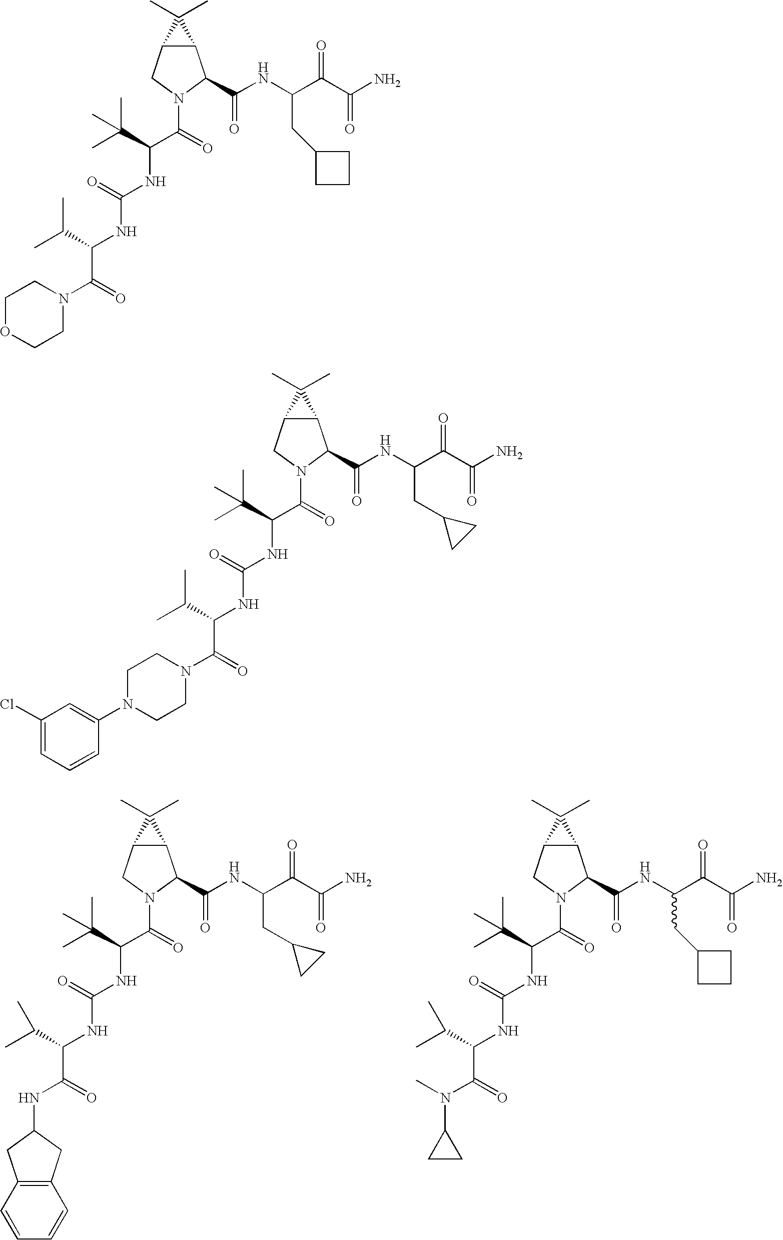 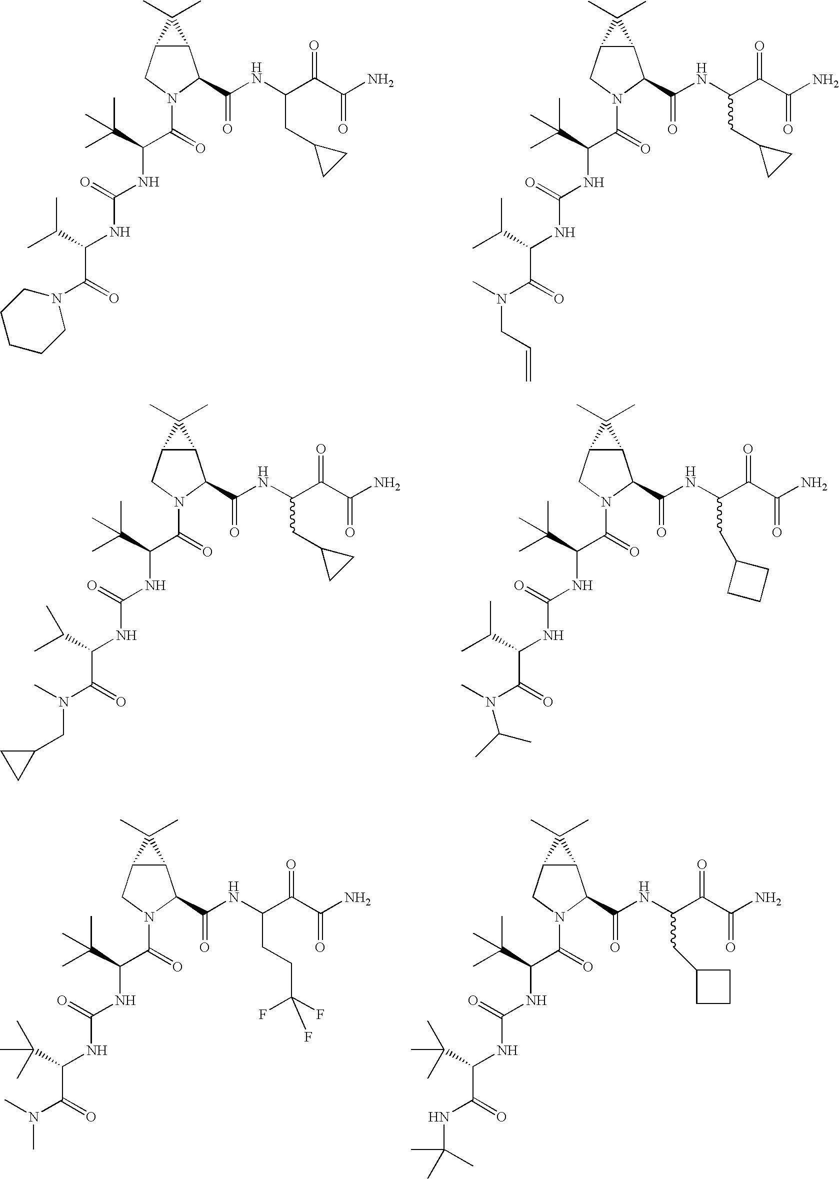 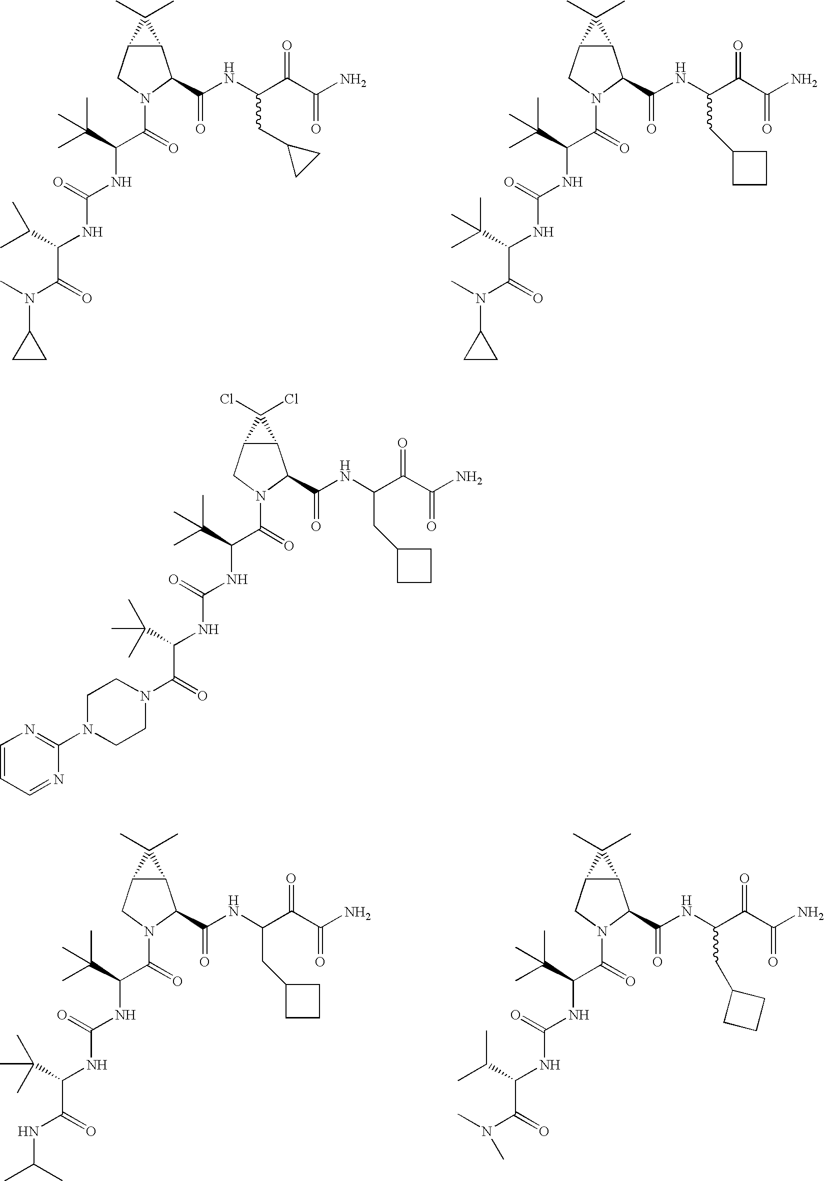 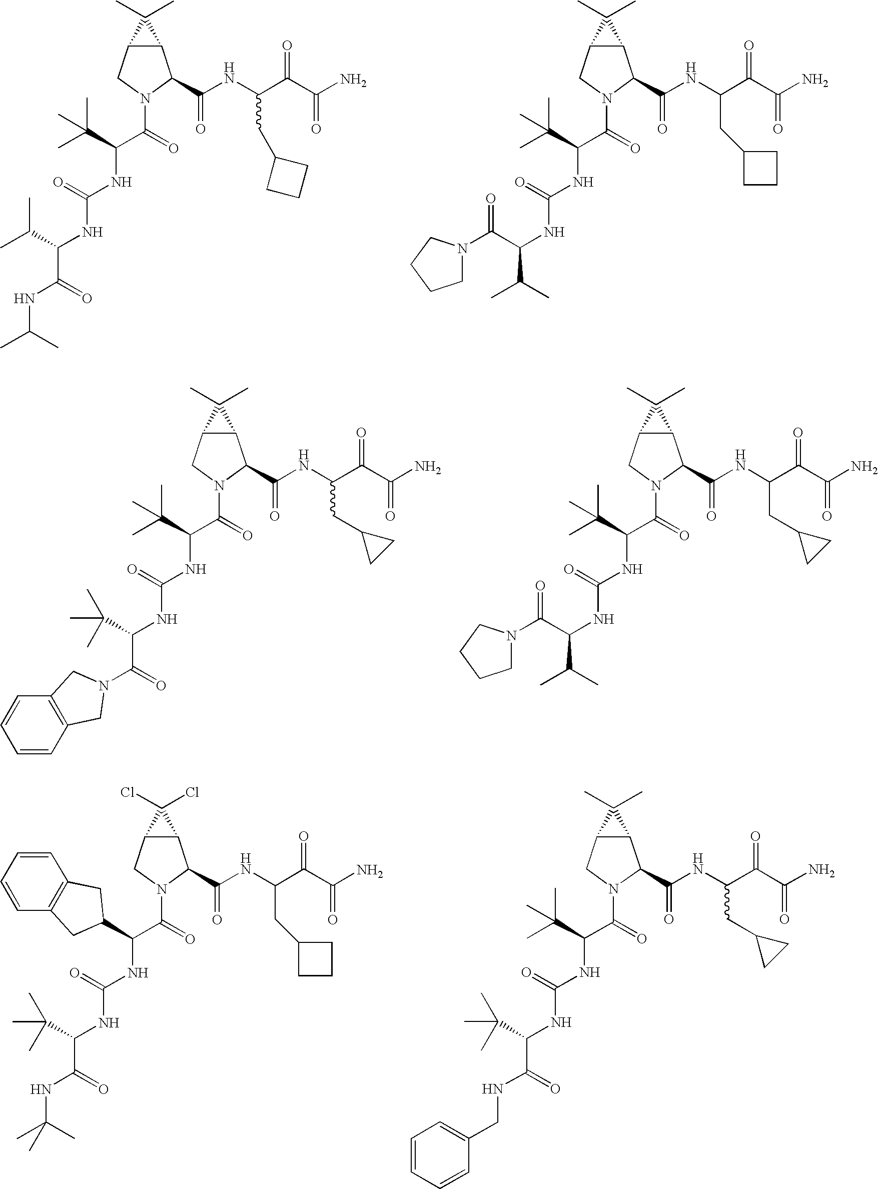 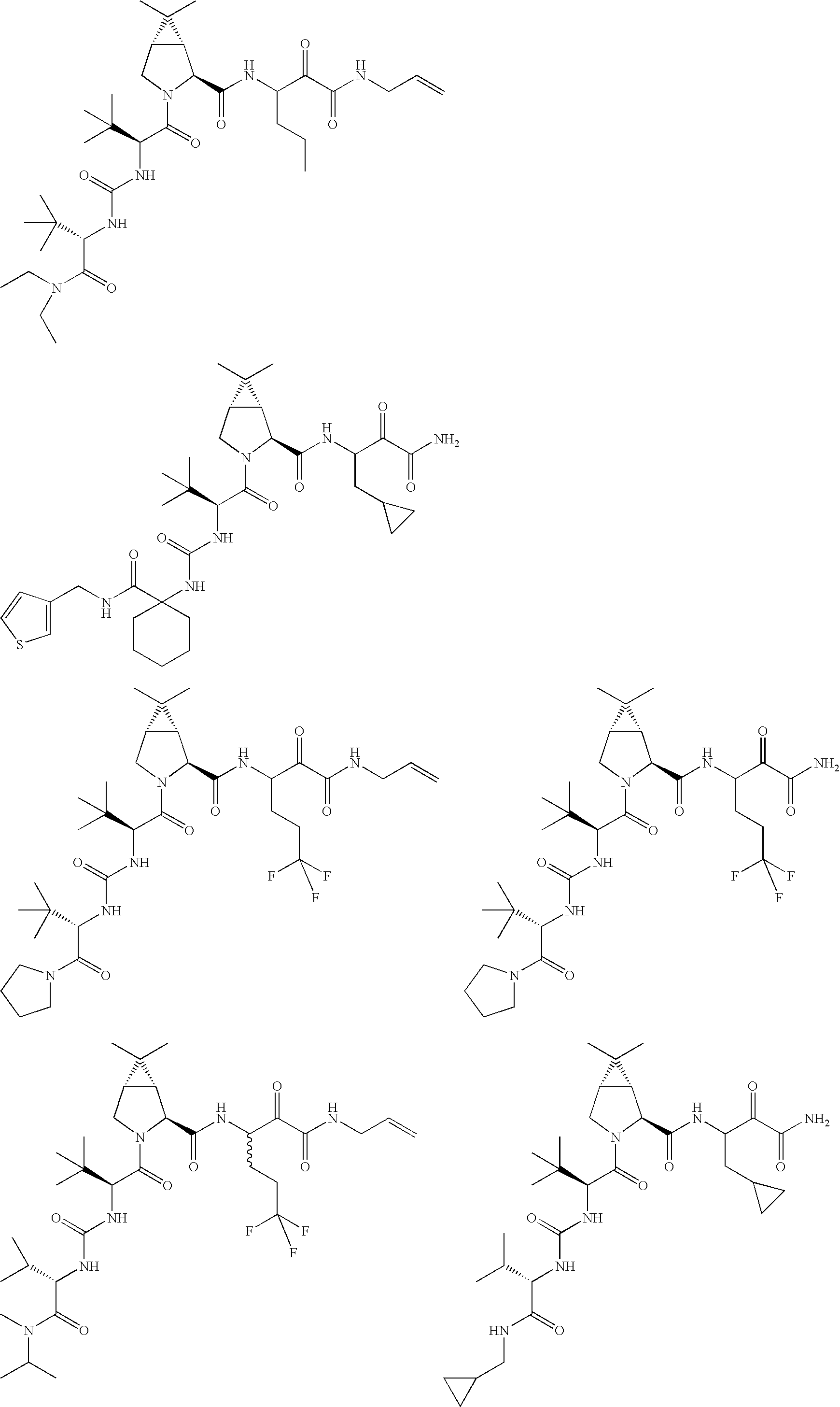 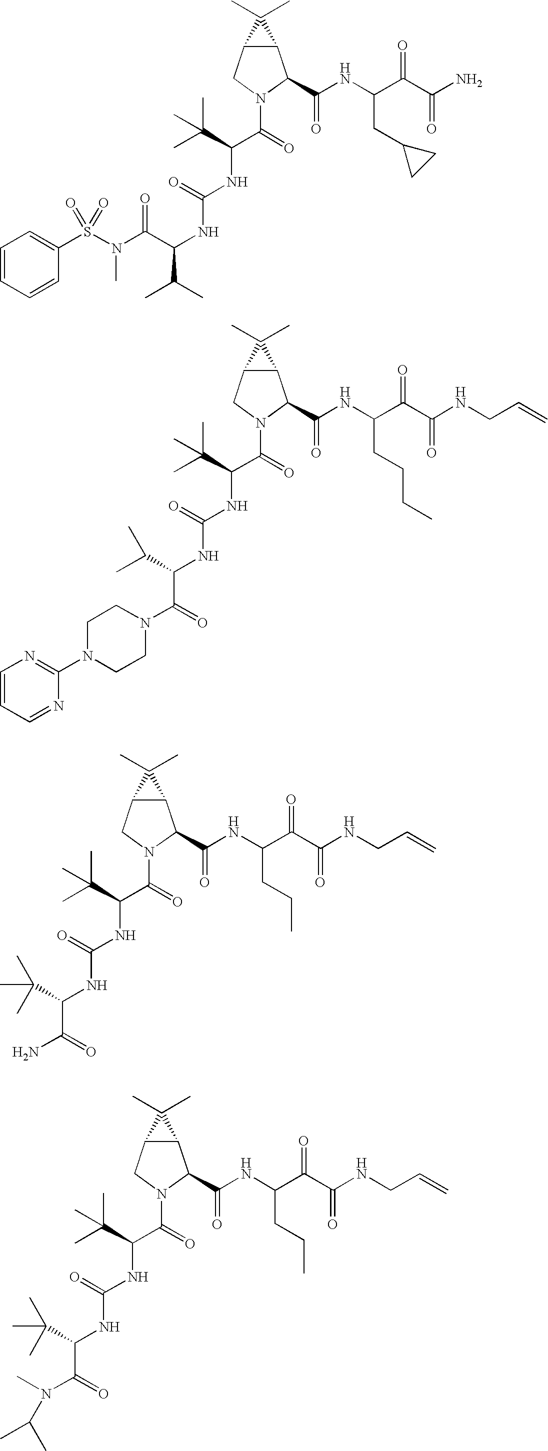 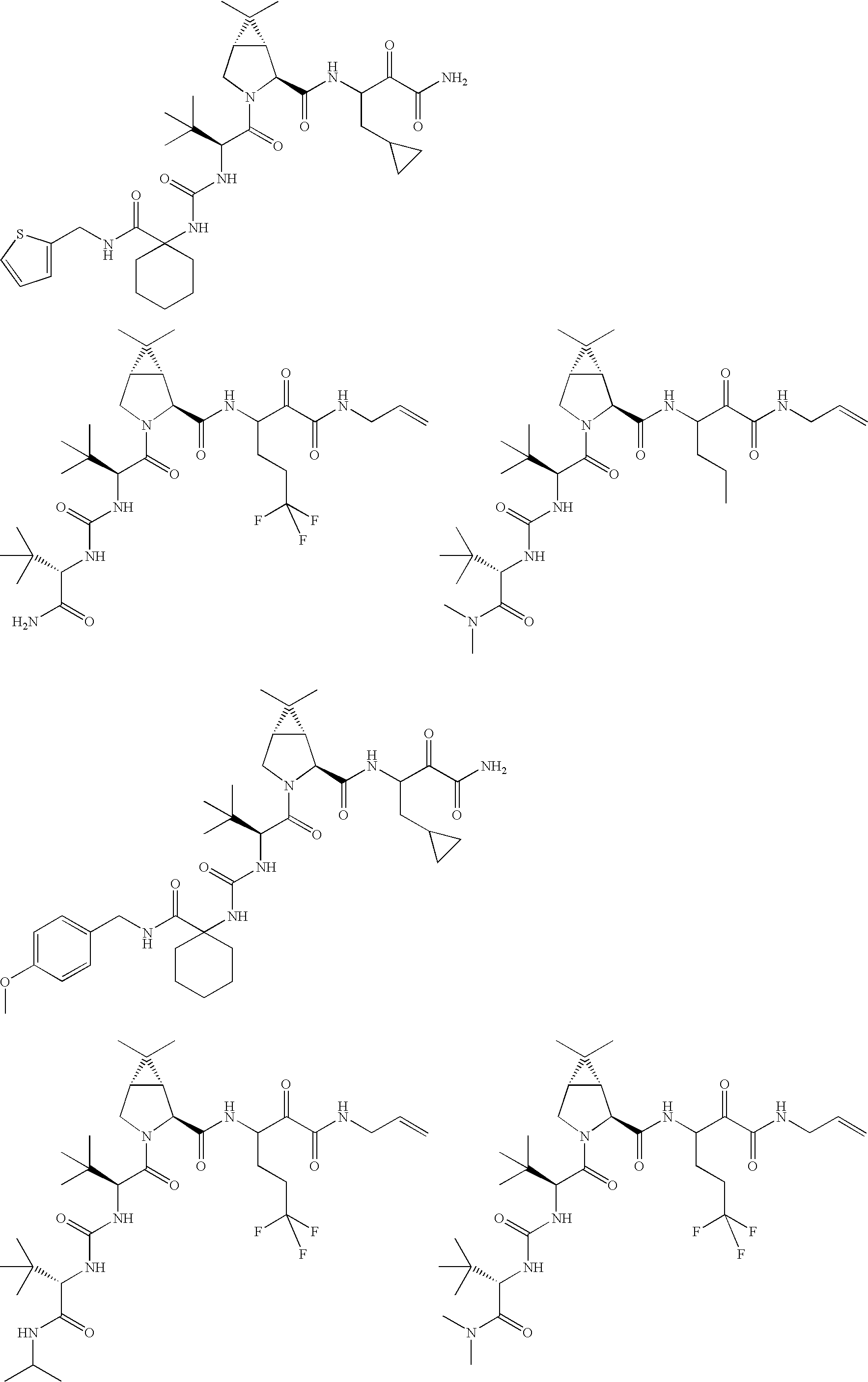 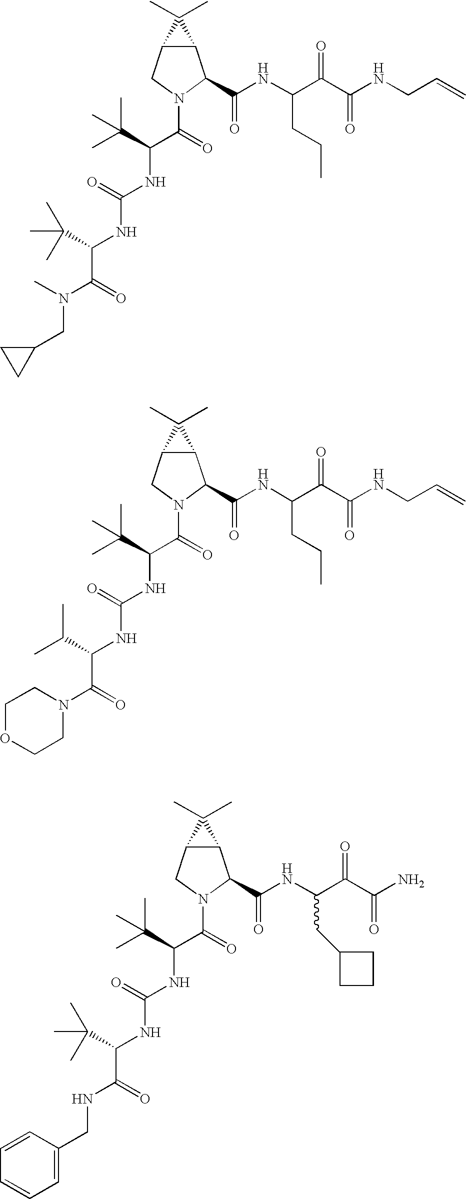 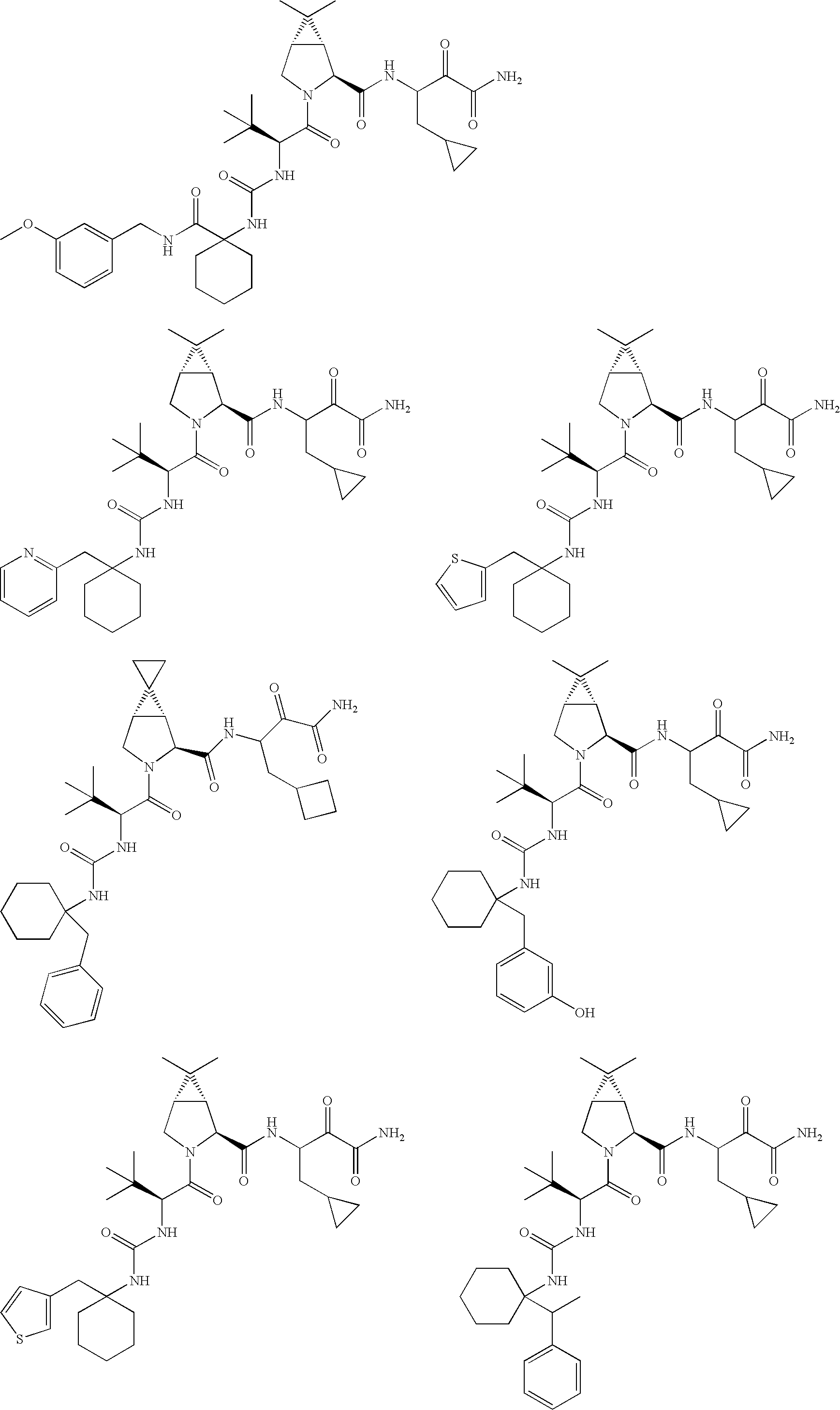 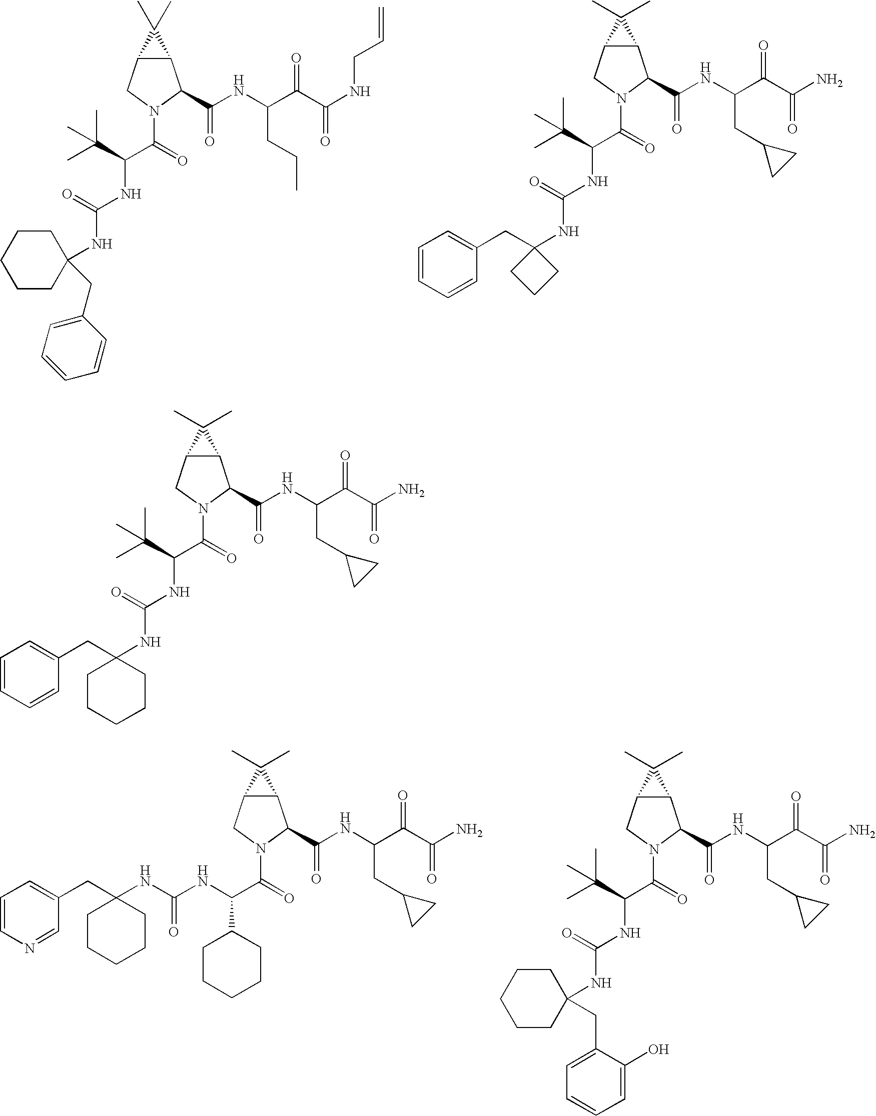 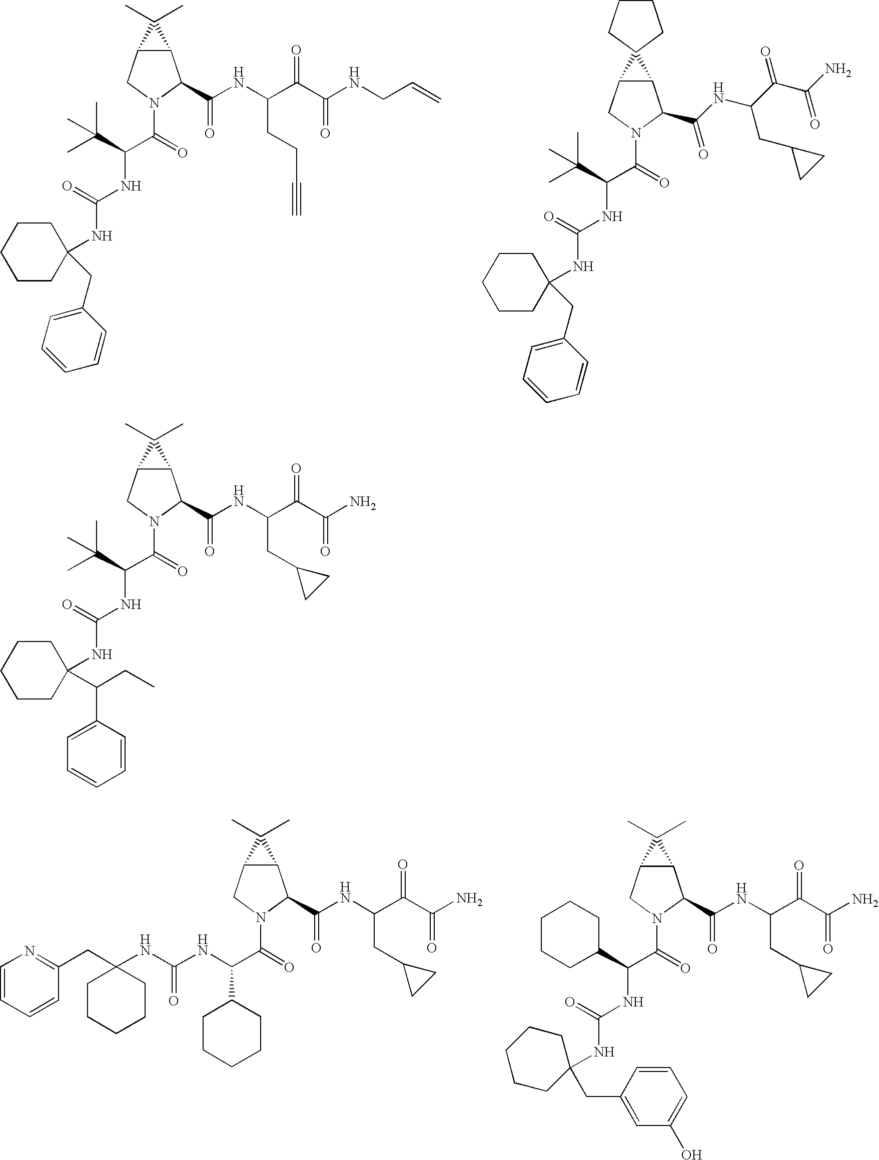 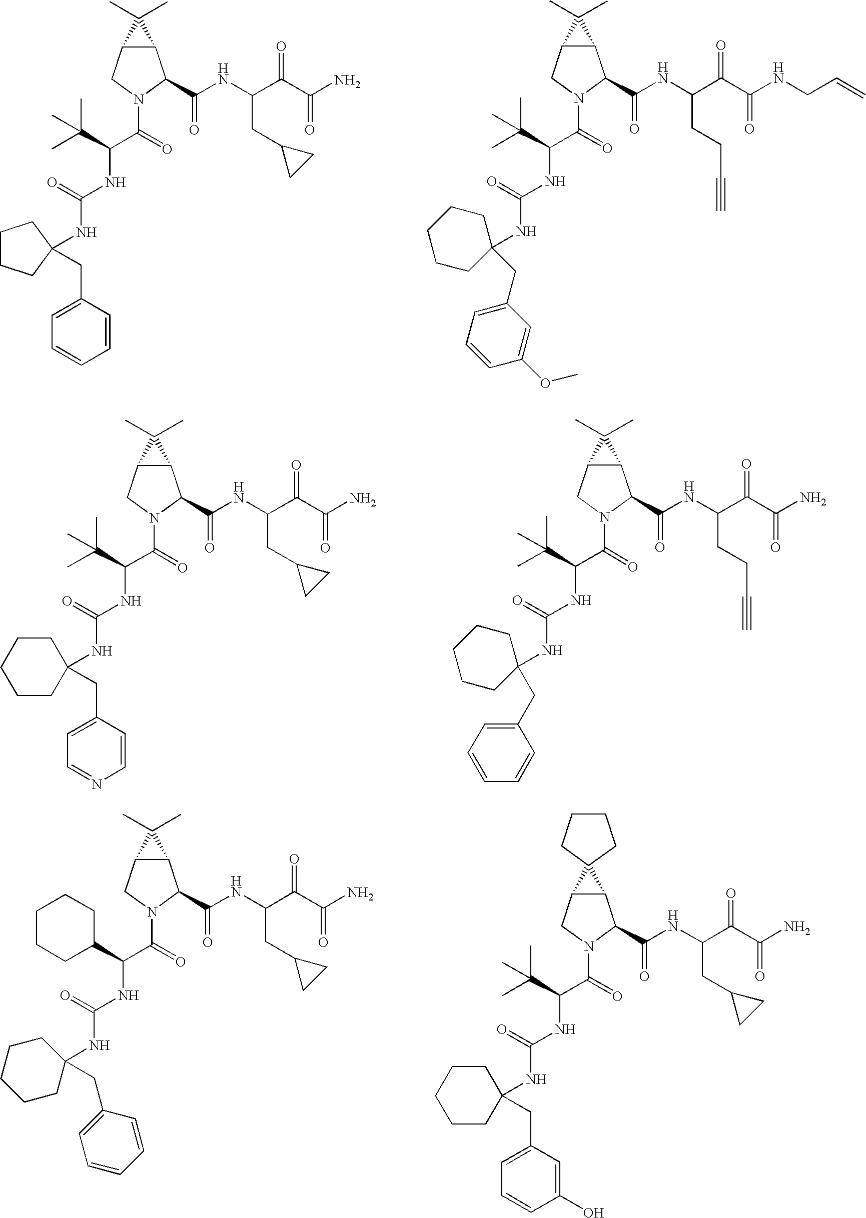 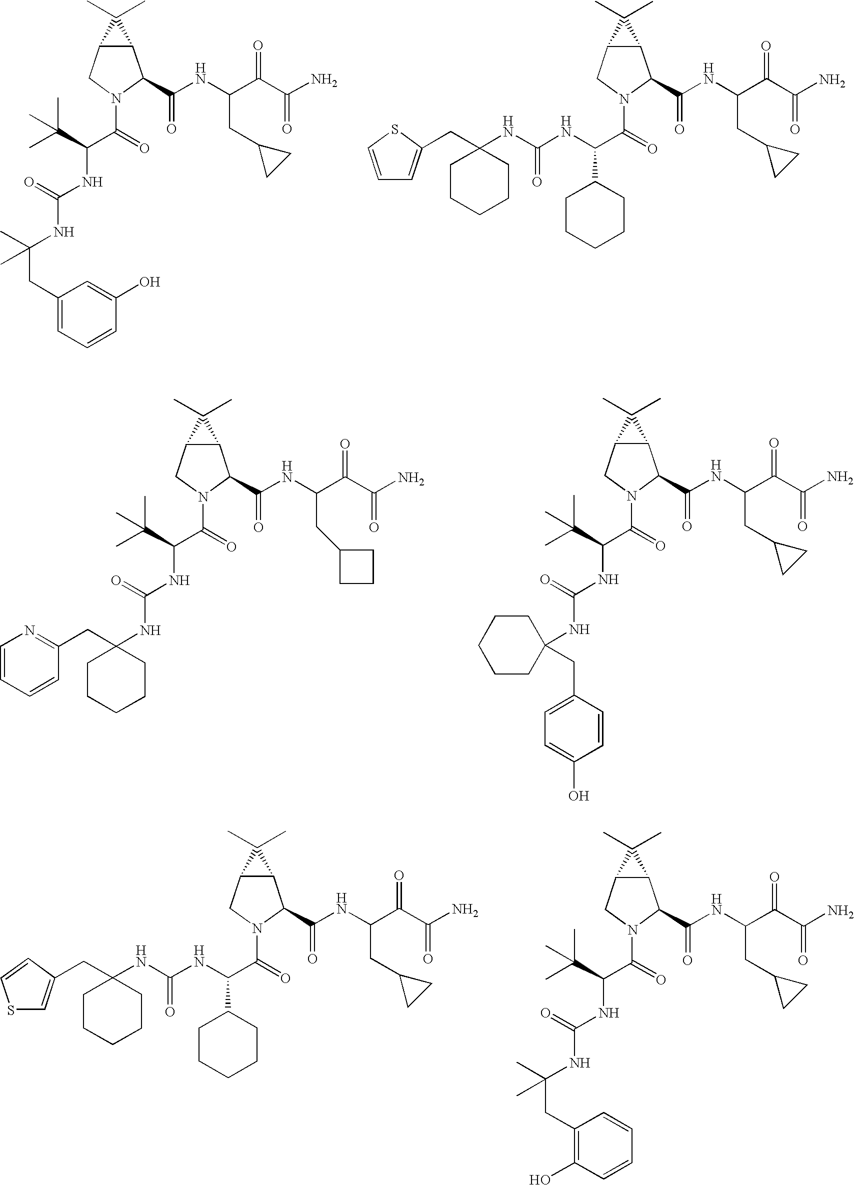 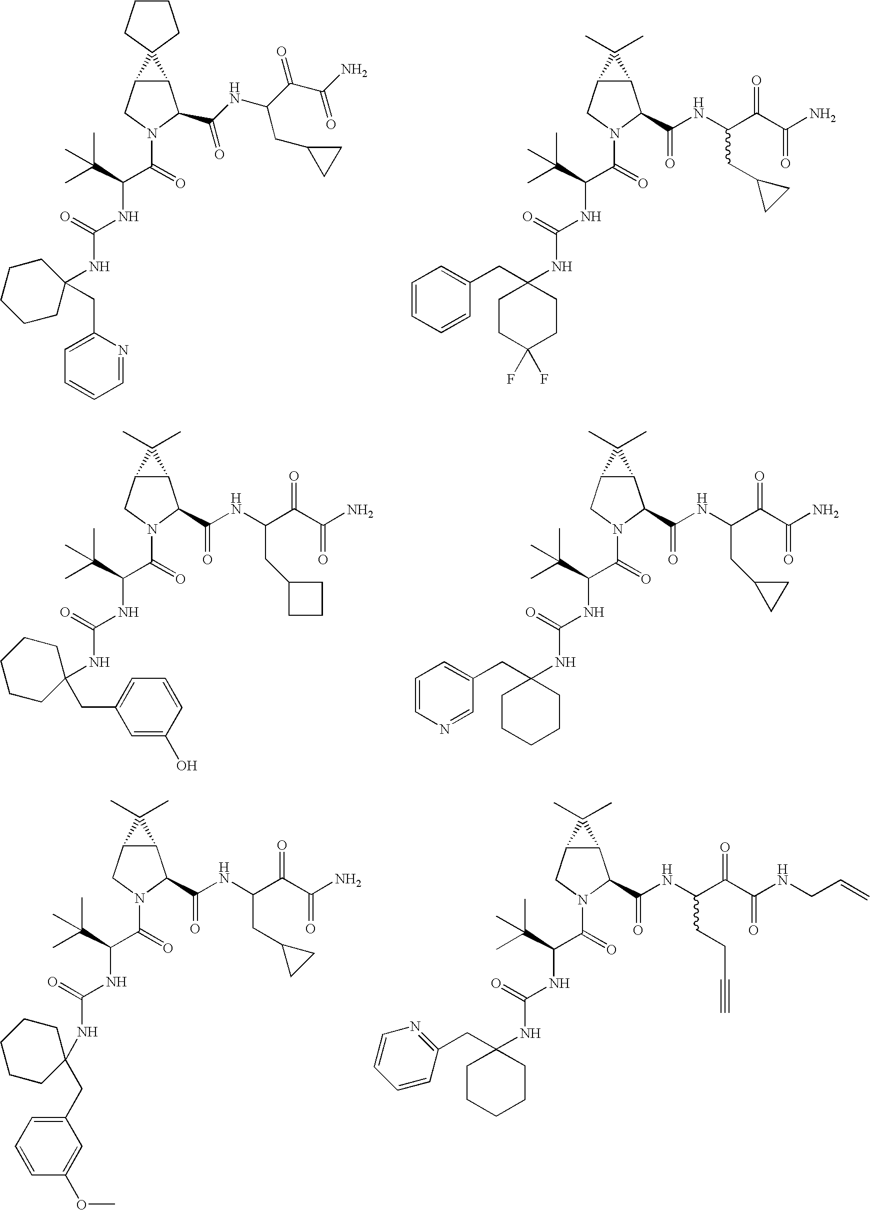 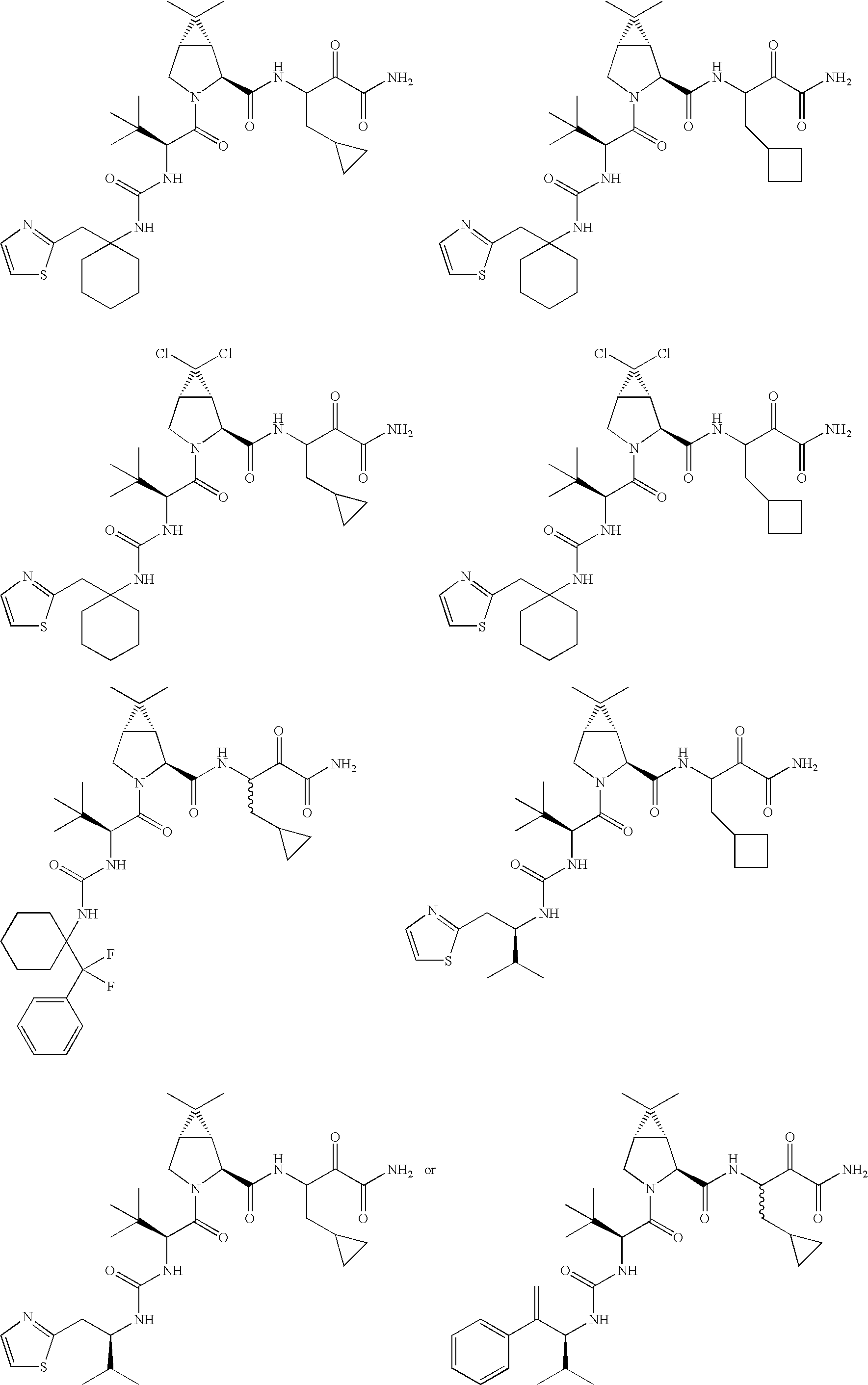
or a pharmaceutically acceptable salt, solvate or ester thereof.

Compounds of formula XI are disclosed in U.S. Patent Application Publication US 2005/0288233, published Dec. 29, 2005, and the corresponding PCT Publication WO2005/087721, published Sep. 22, 2005, both of which are incorporated herein by reference.

Non-limiting examples of certain compounds disclosed in these publications are: 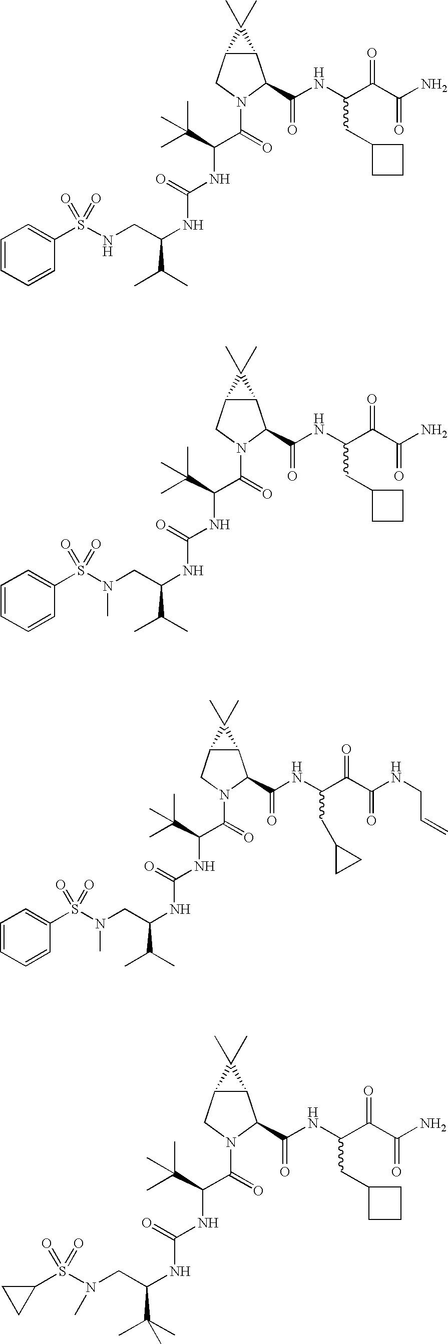 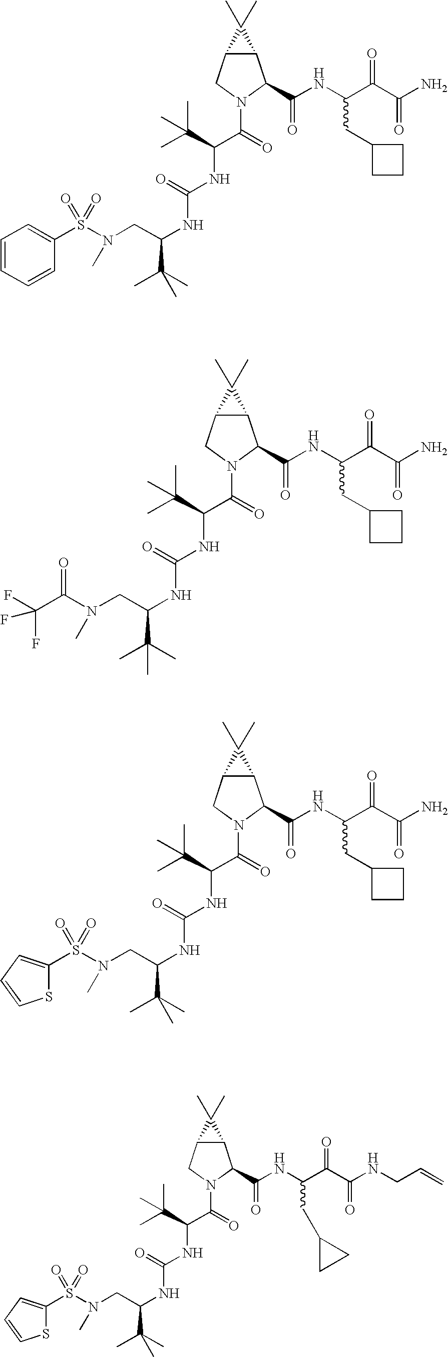 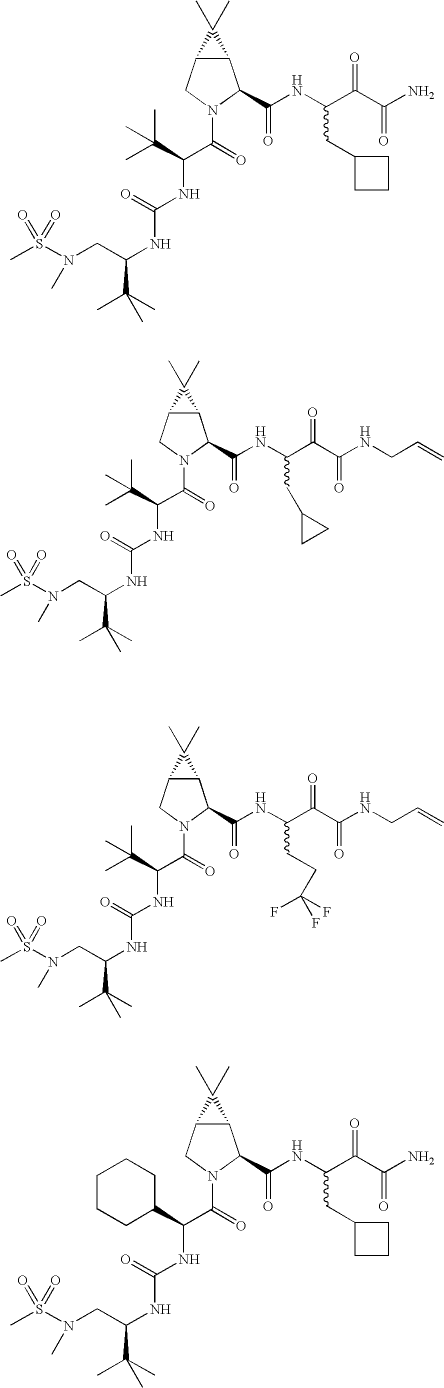 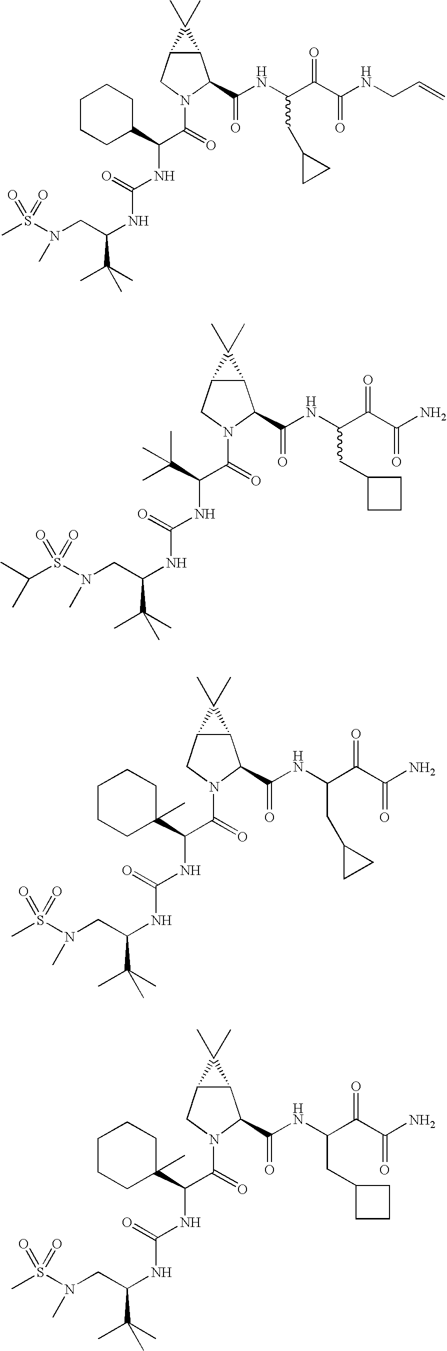 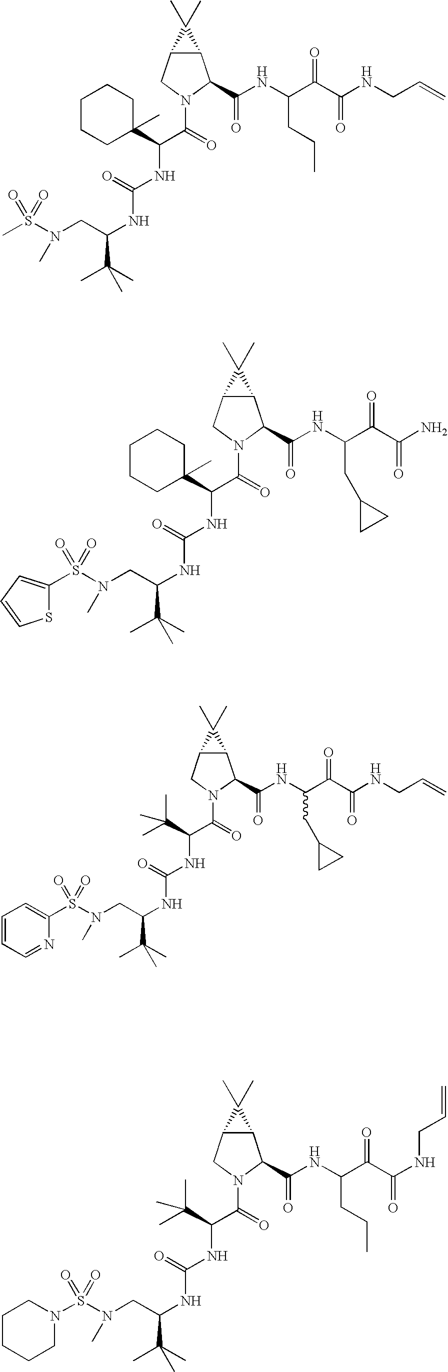 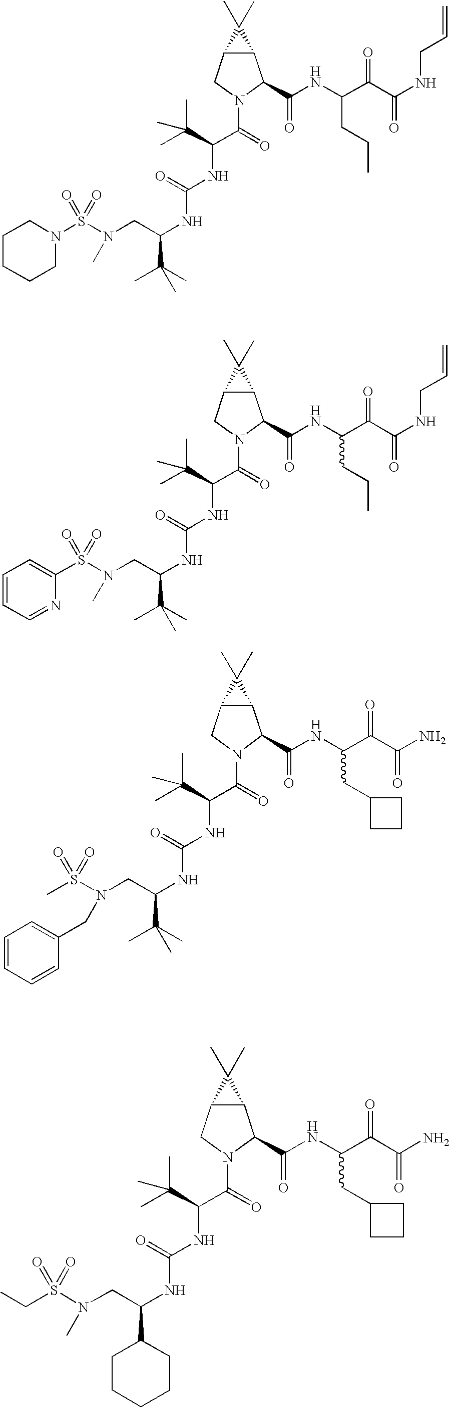 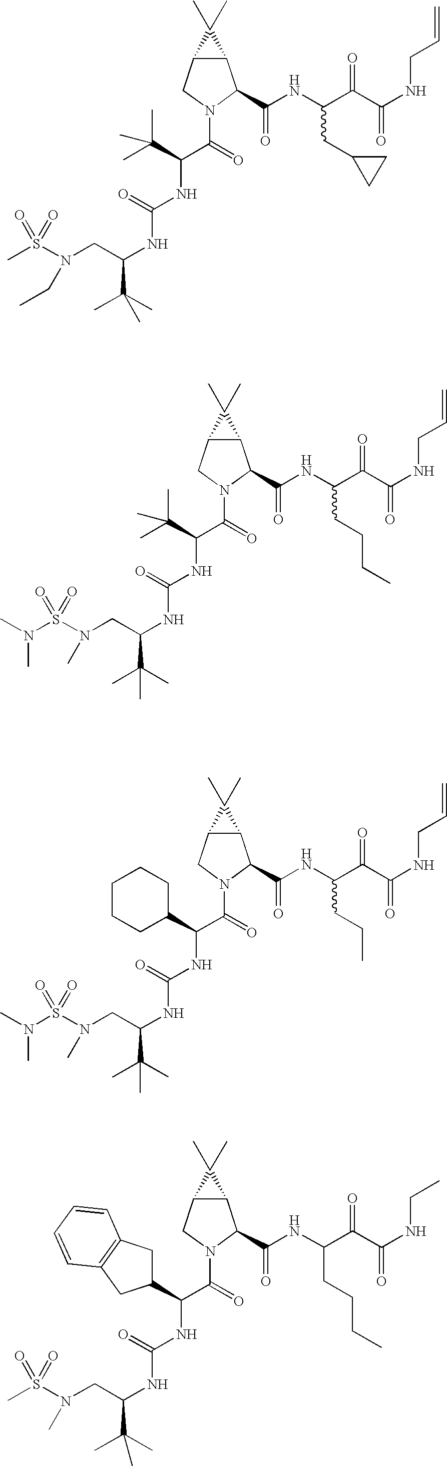 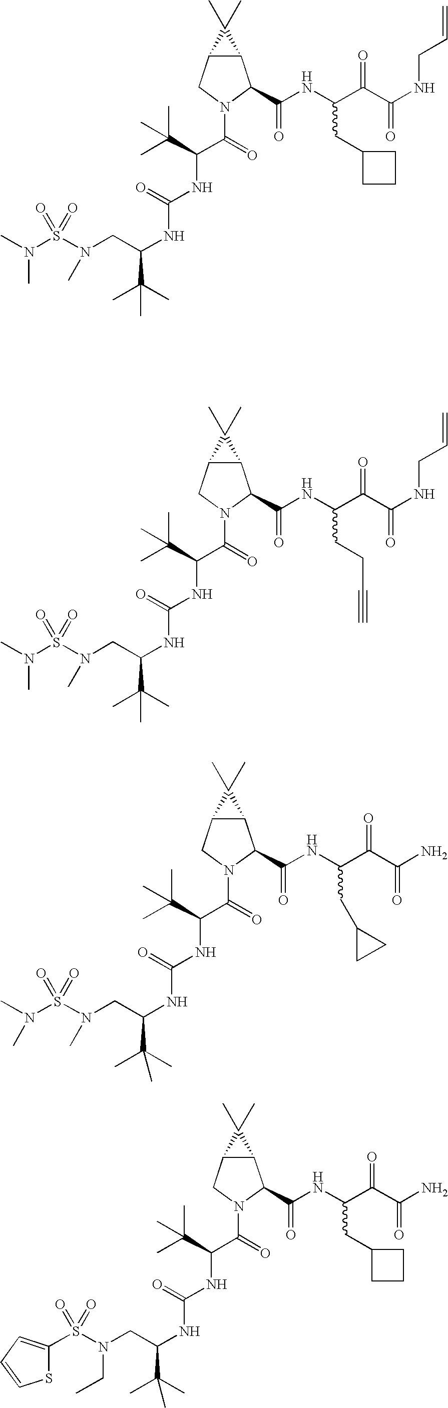 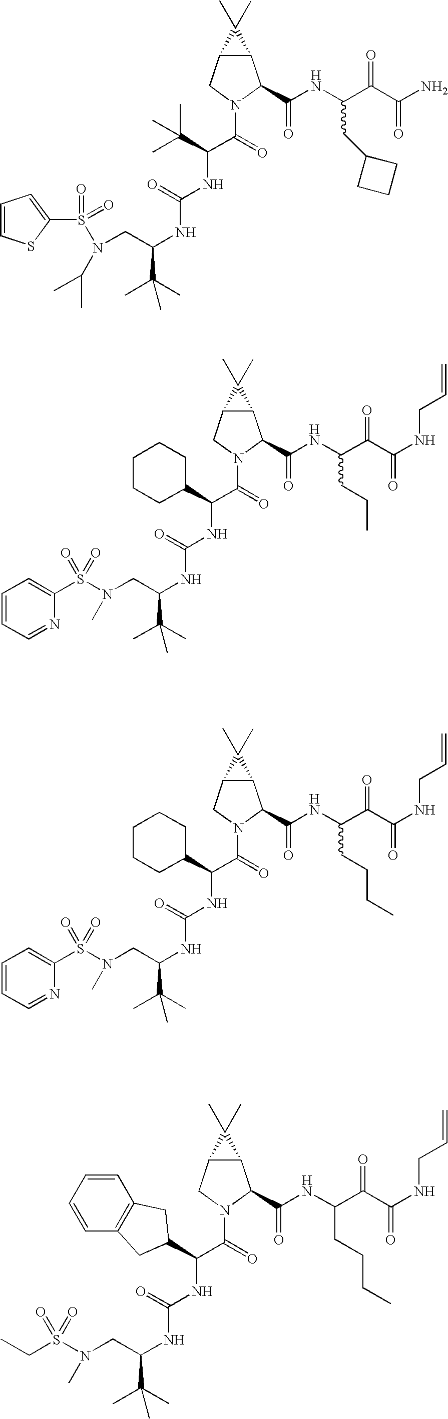 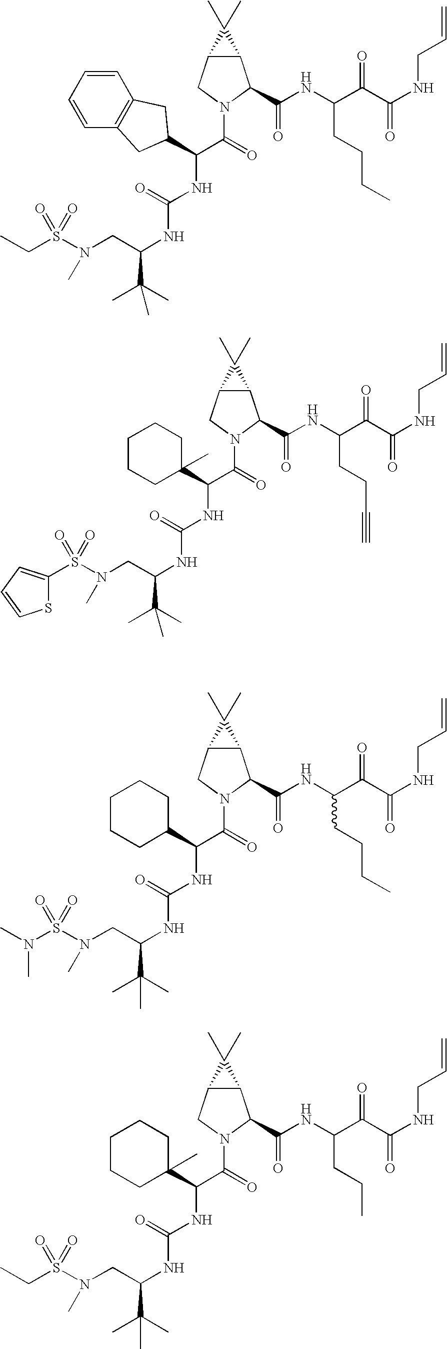 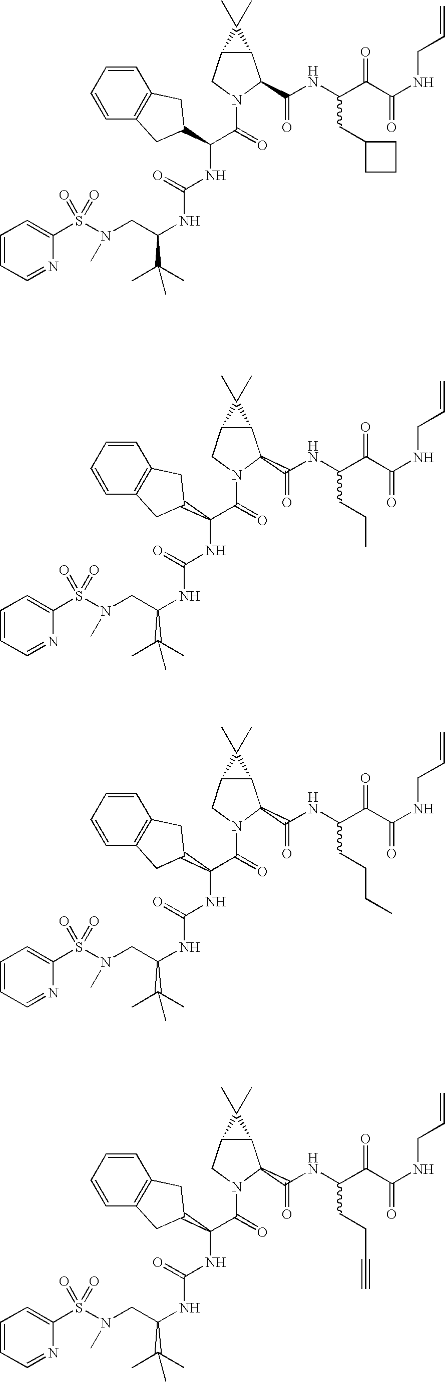 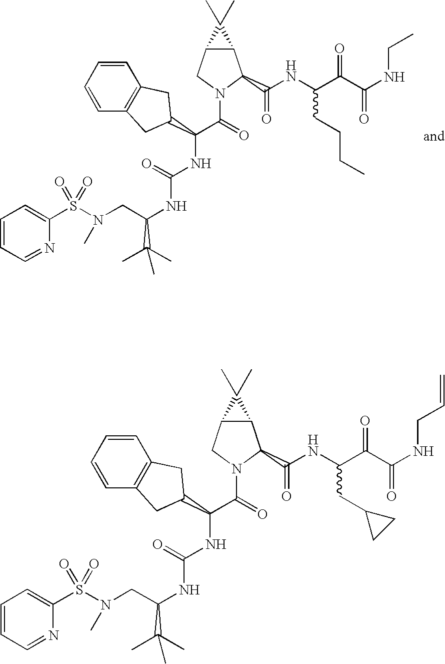
or a pharmaceutically acceptable salt, solvate or ester thereof.

Compounds of formula XII are disclosed in U.S. Patent Application Publication US 2005/0245458, published Nov. 3, 2005, and the corresponding PCT publication WO2005/087725, published Sep.22, 2005, both of which are incorporated herein by reference.

Non-limiting examples of certain compounds disclosed in these publications are: 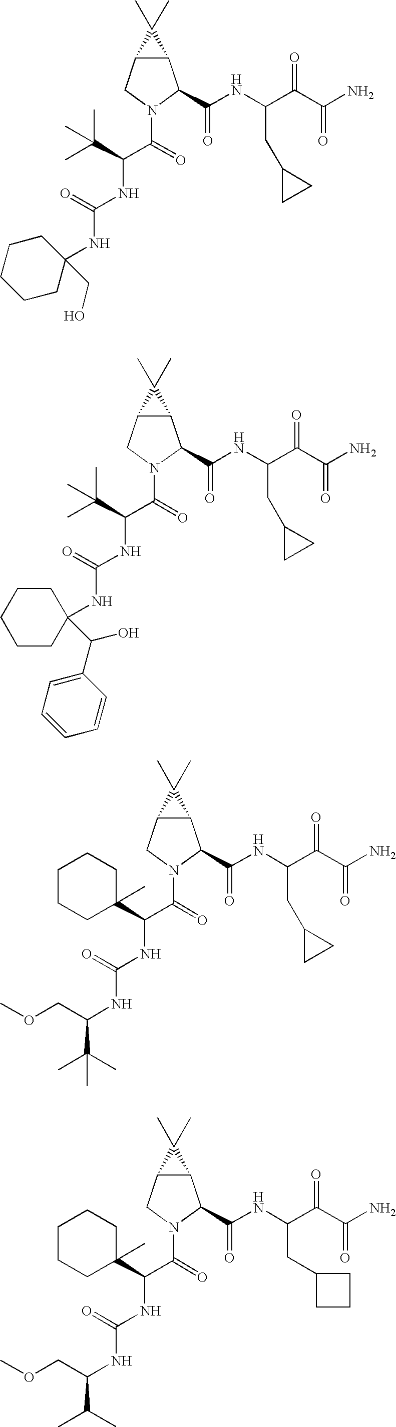 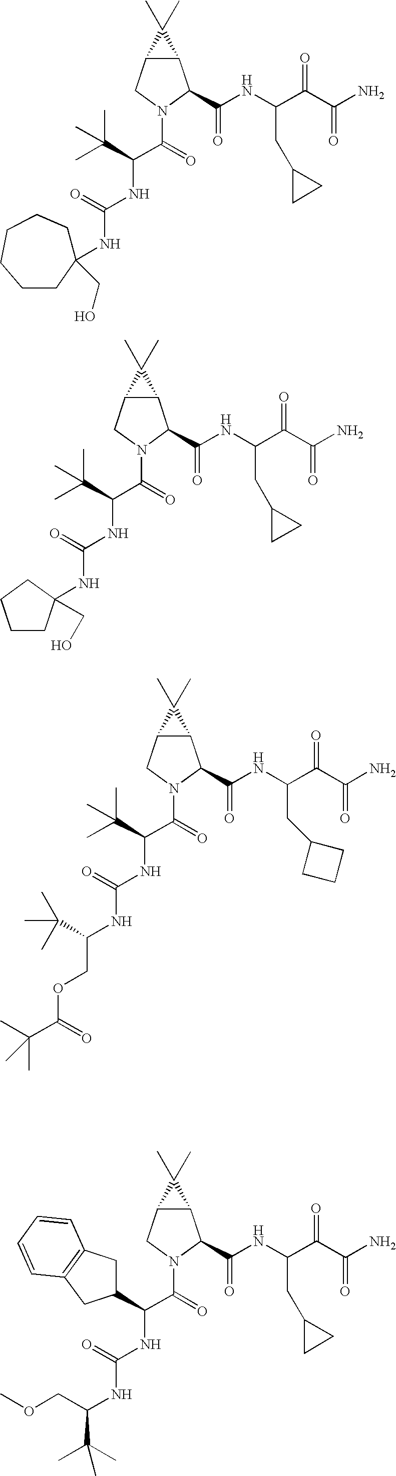 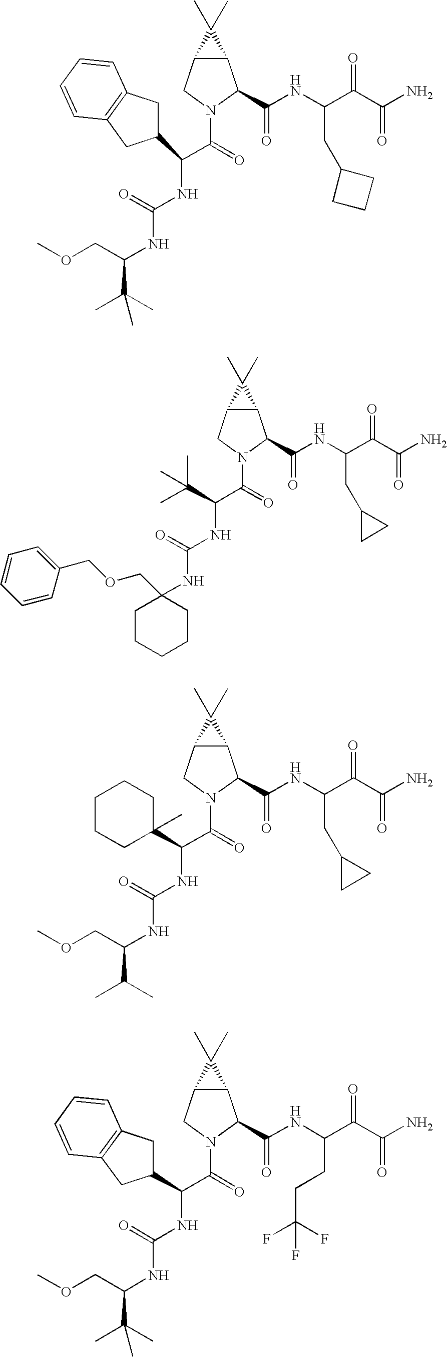 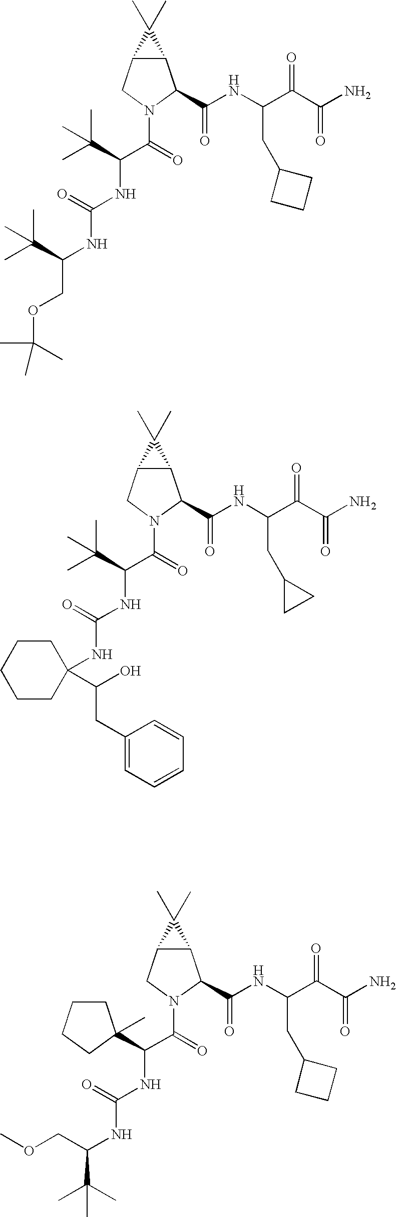 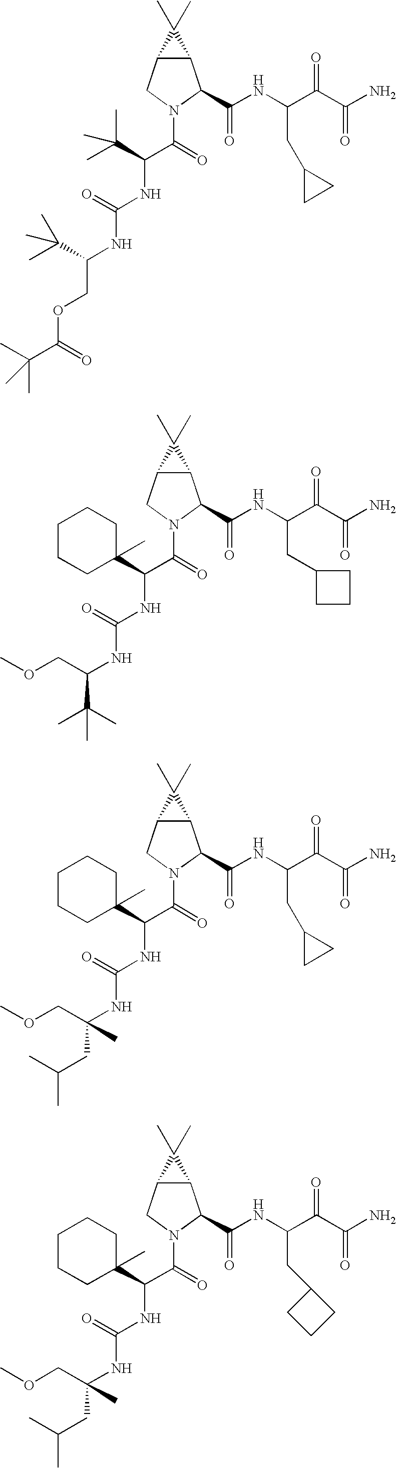 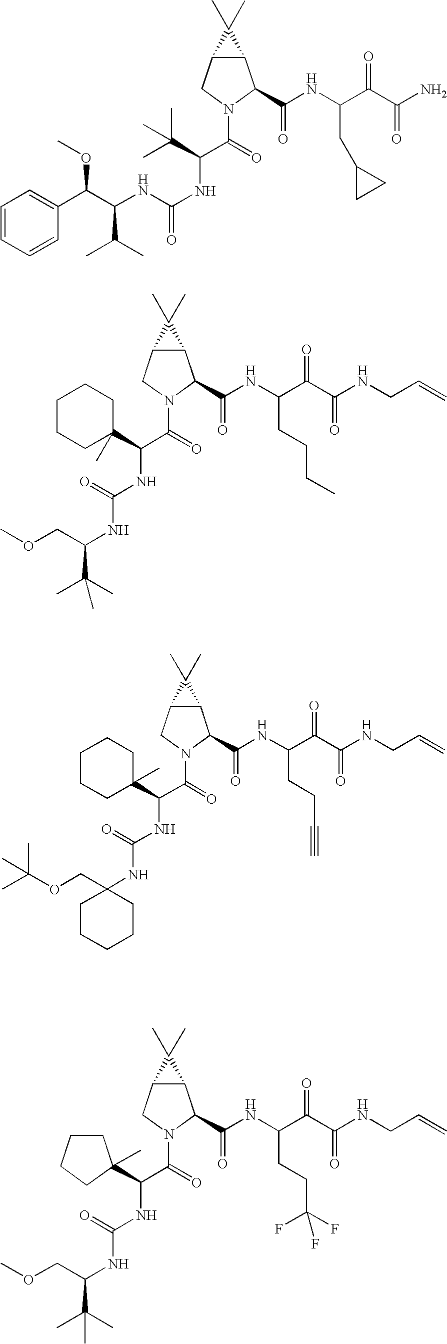 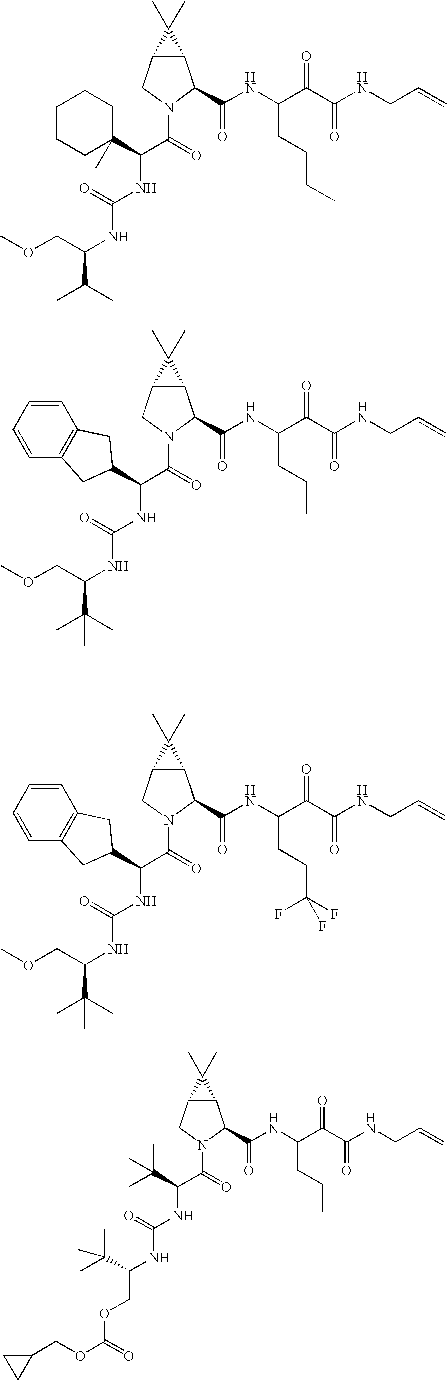 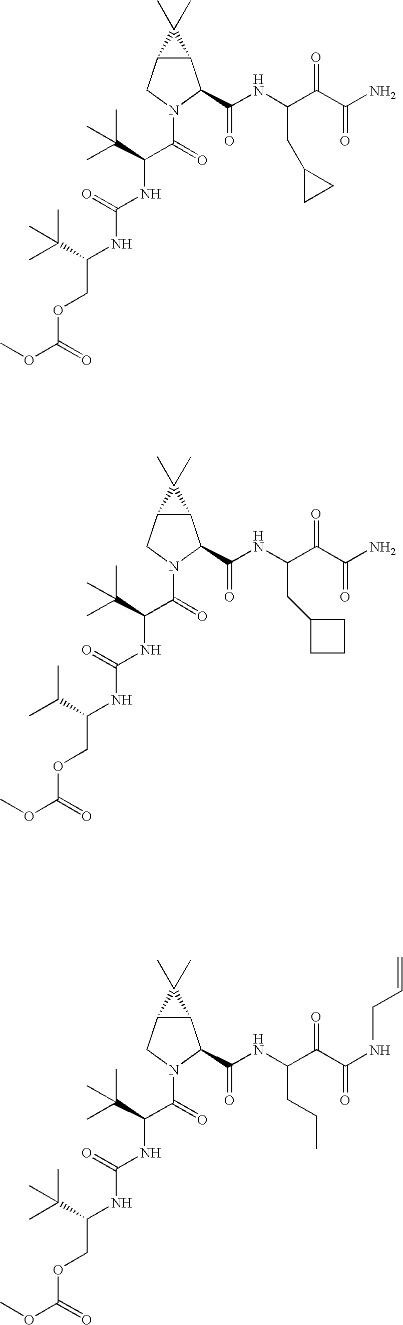 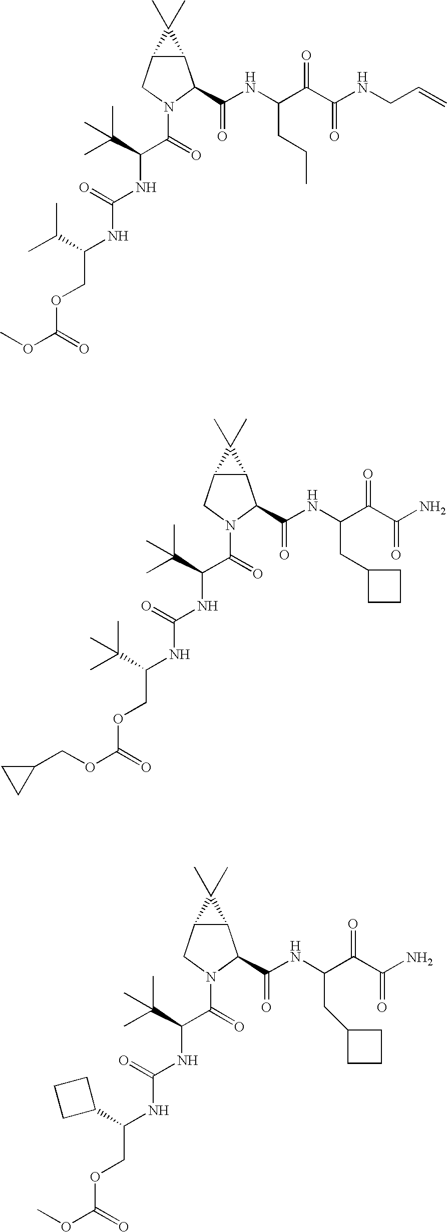 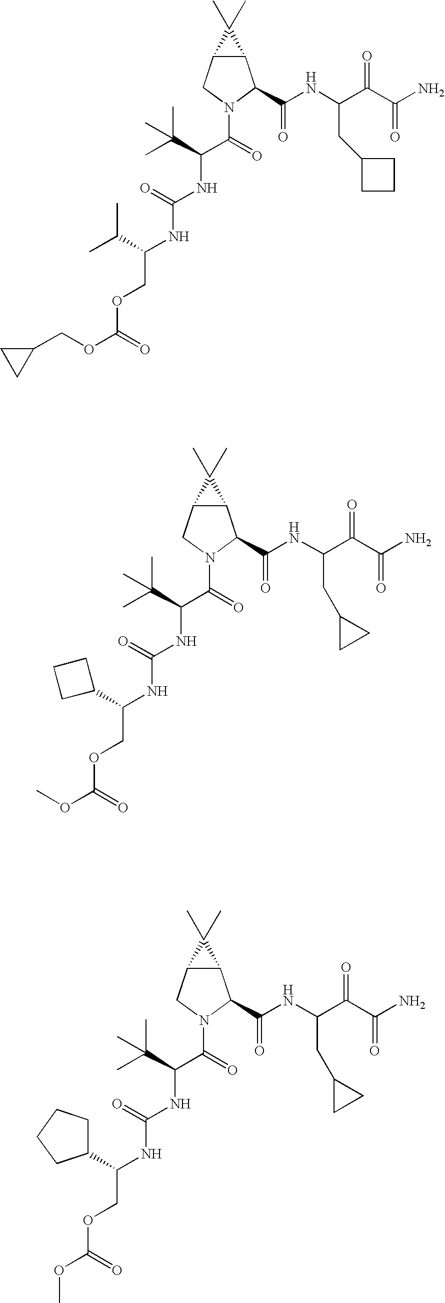 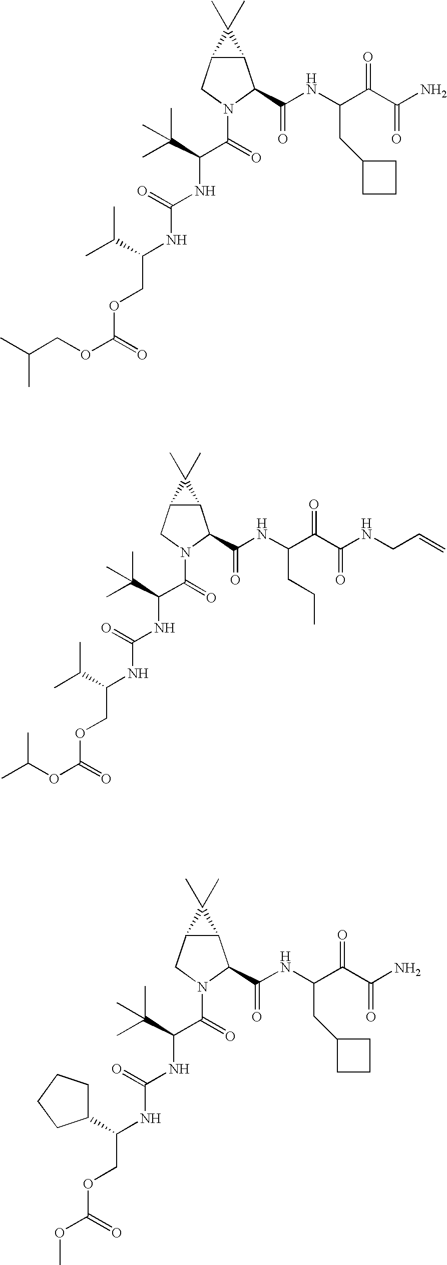 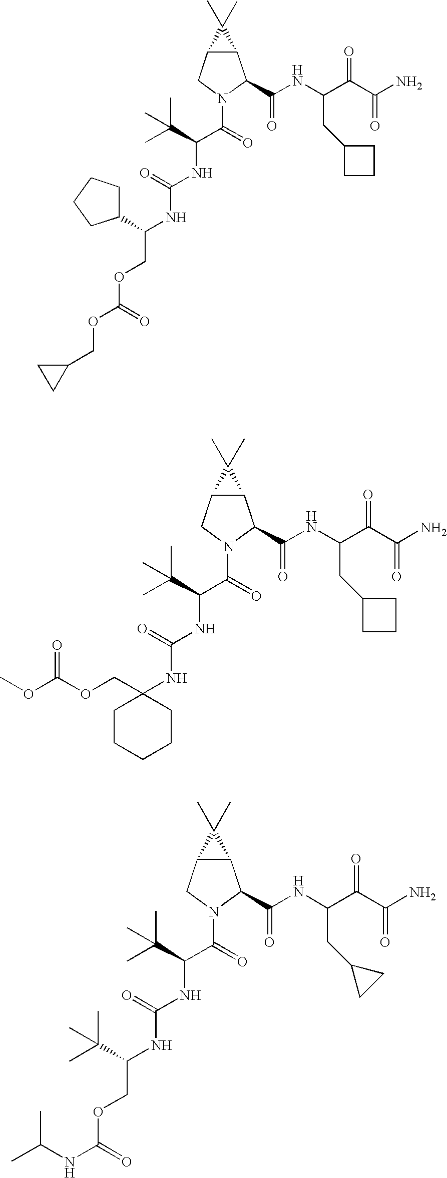 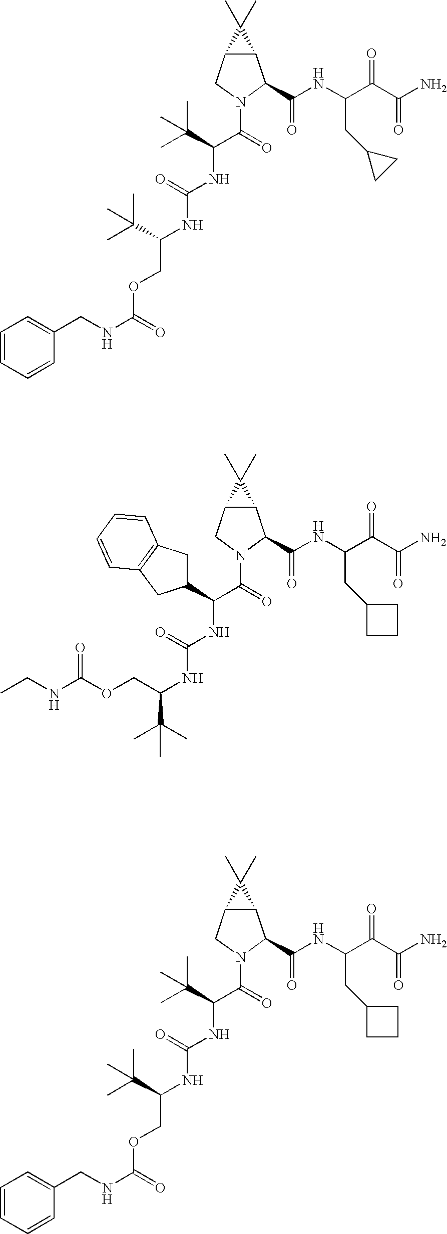 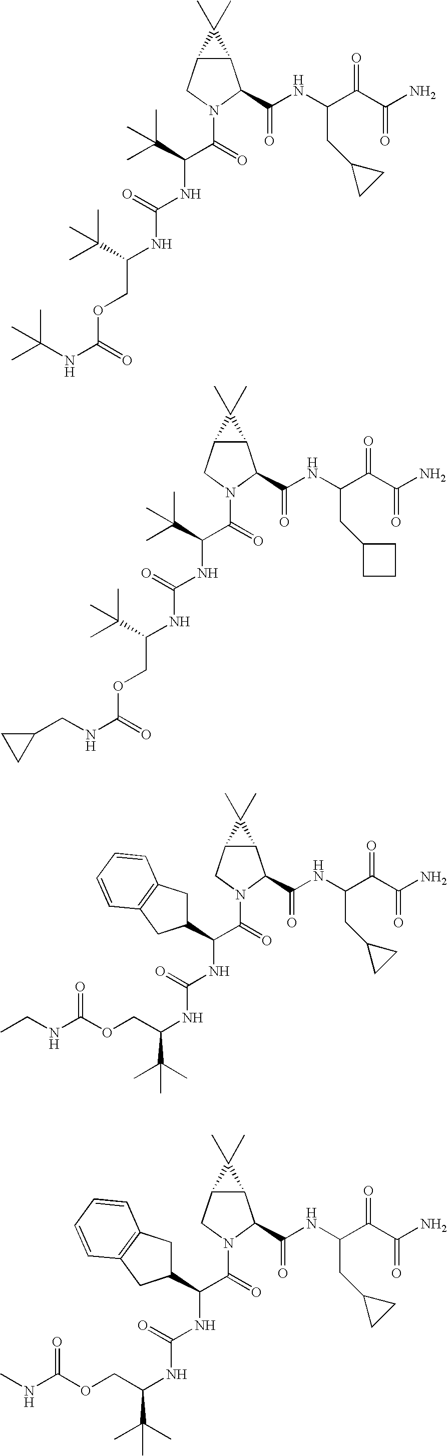 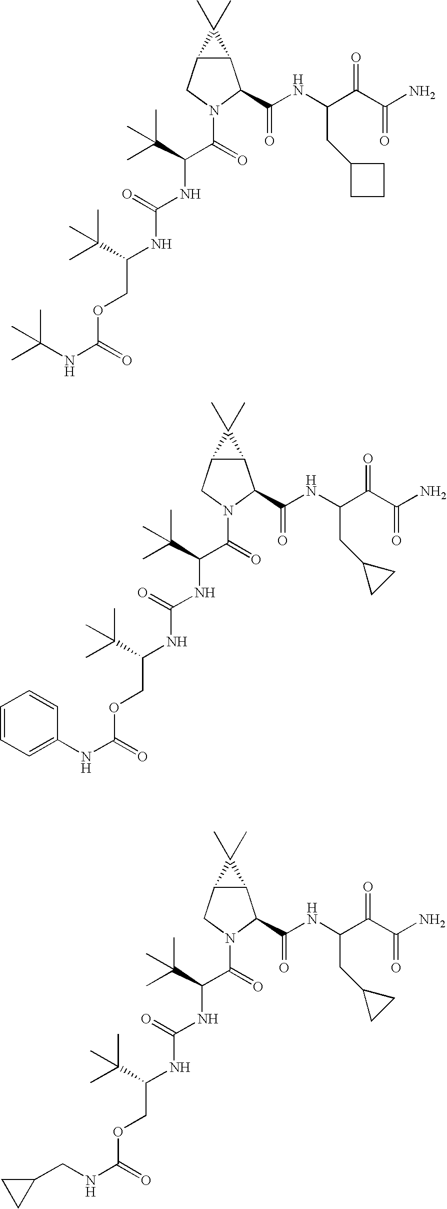 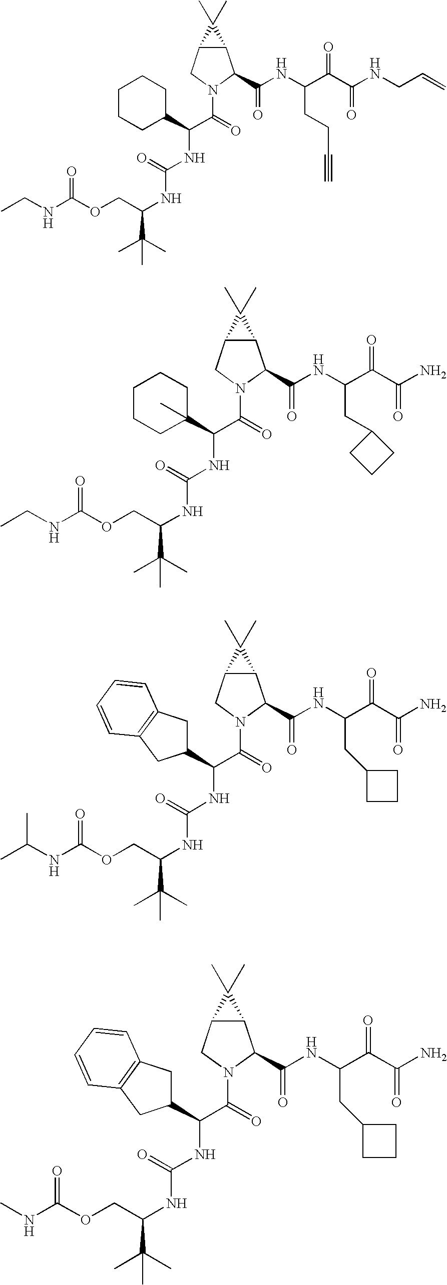 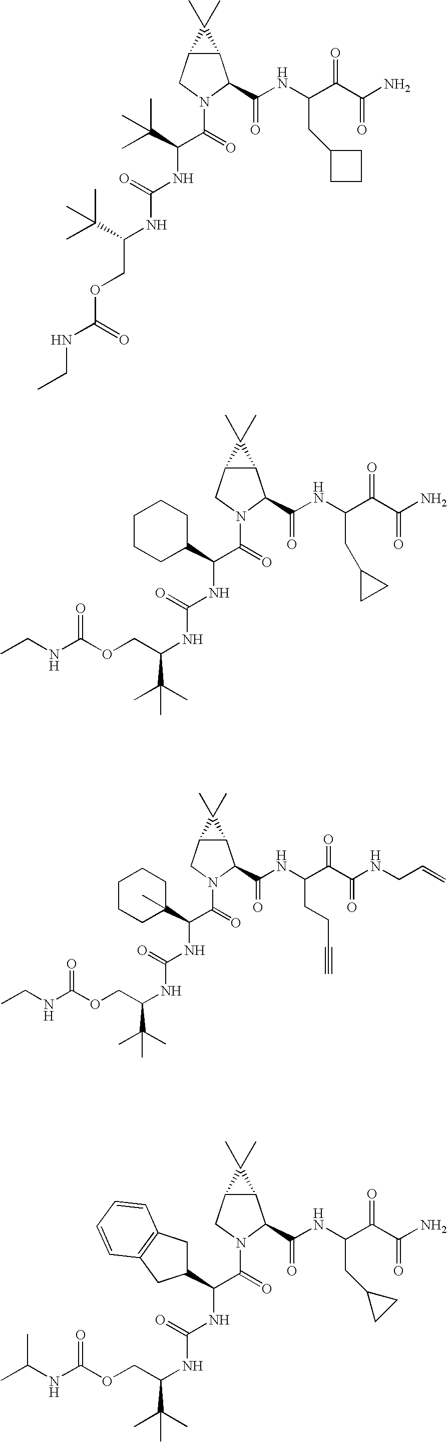 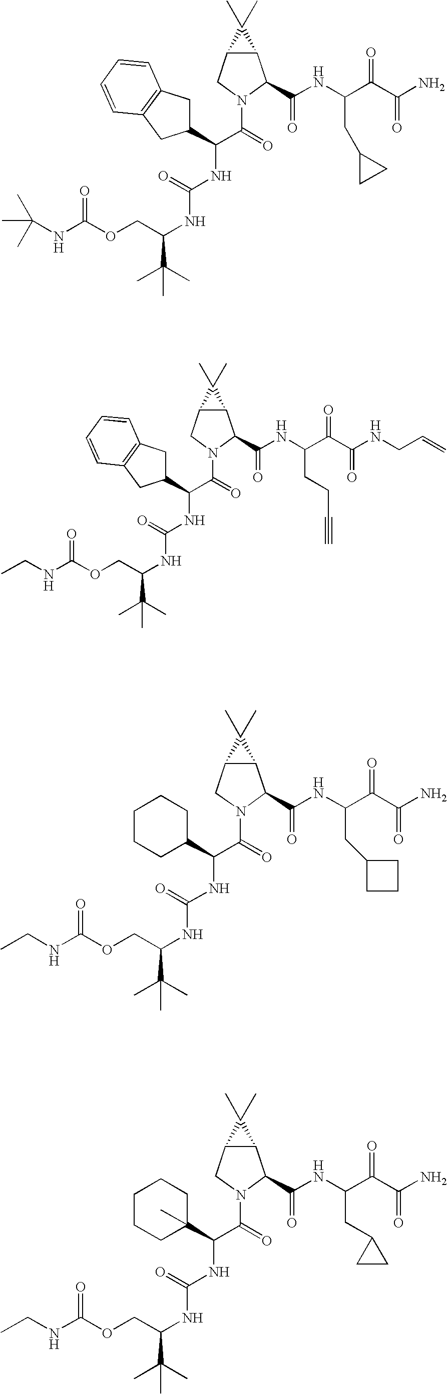 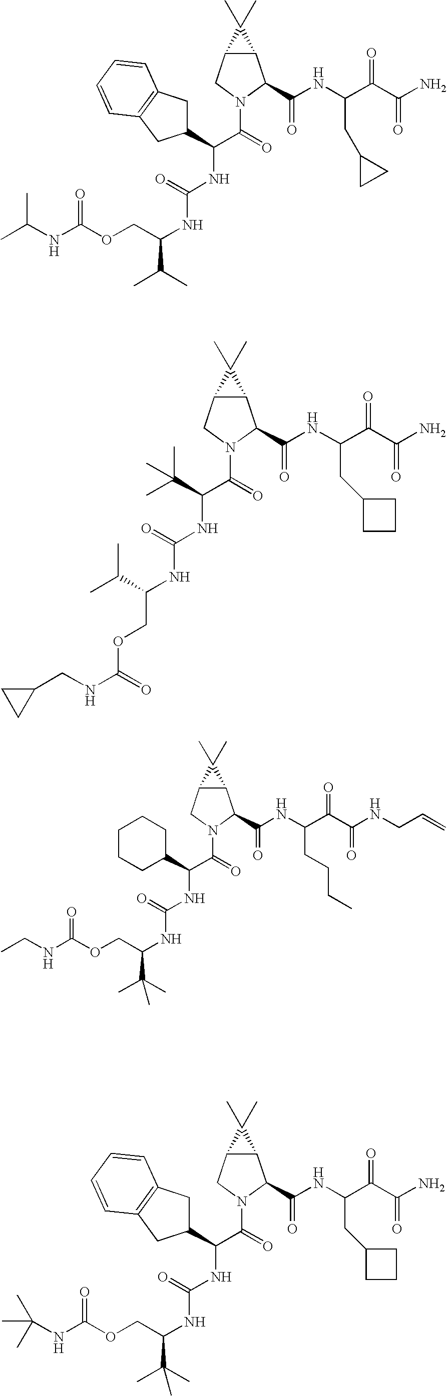 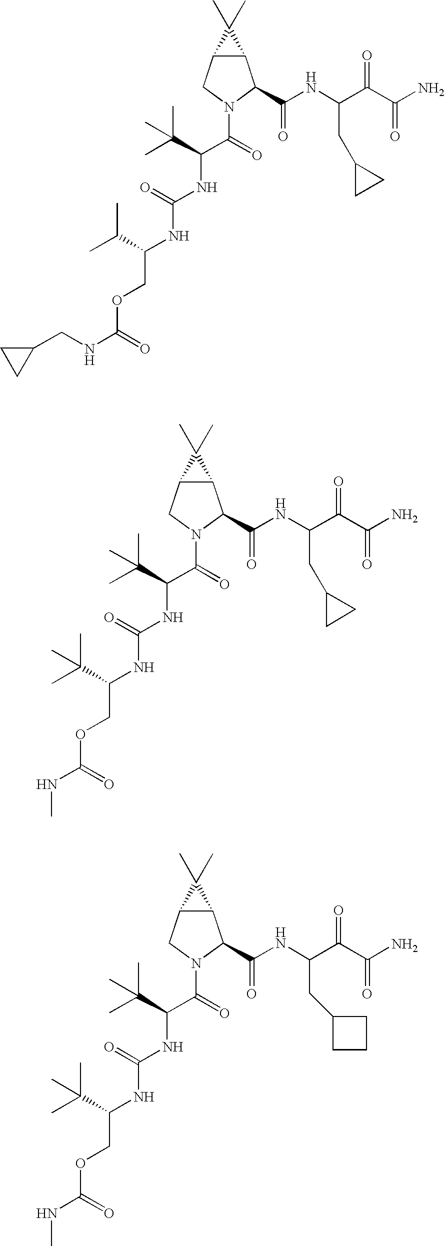 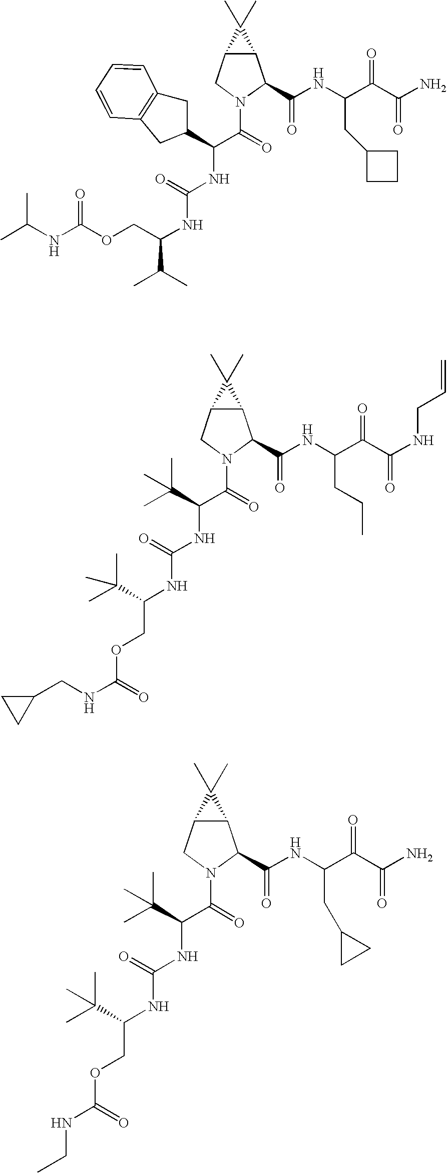 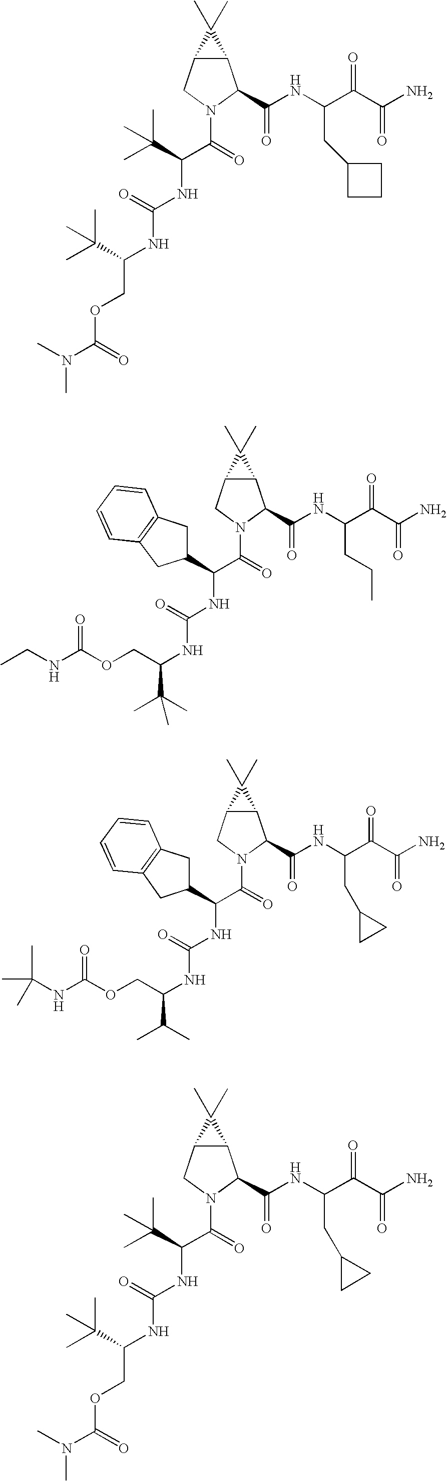 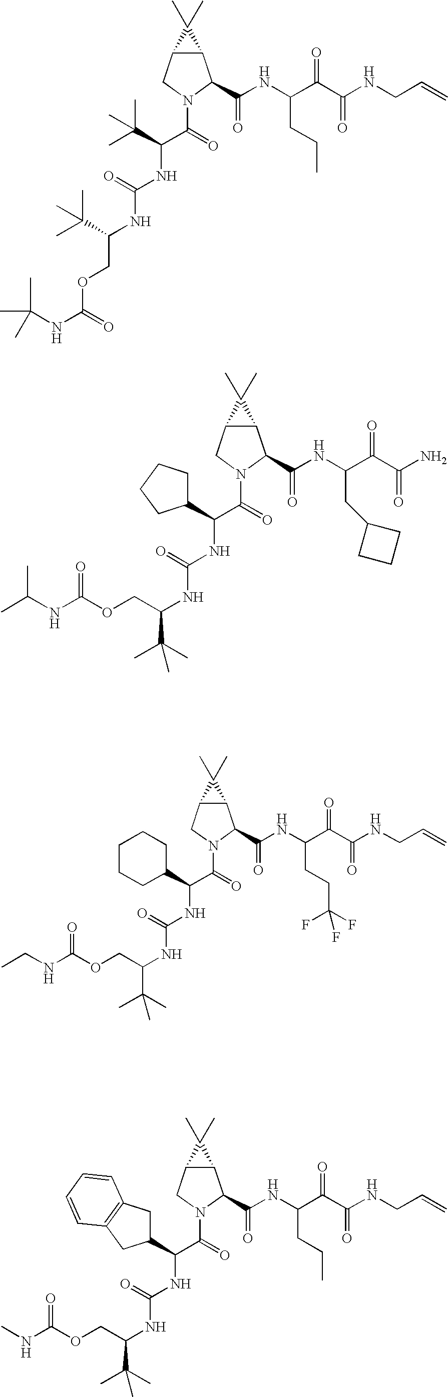 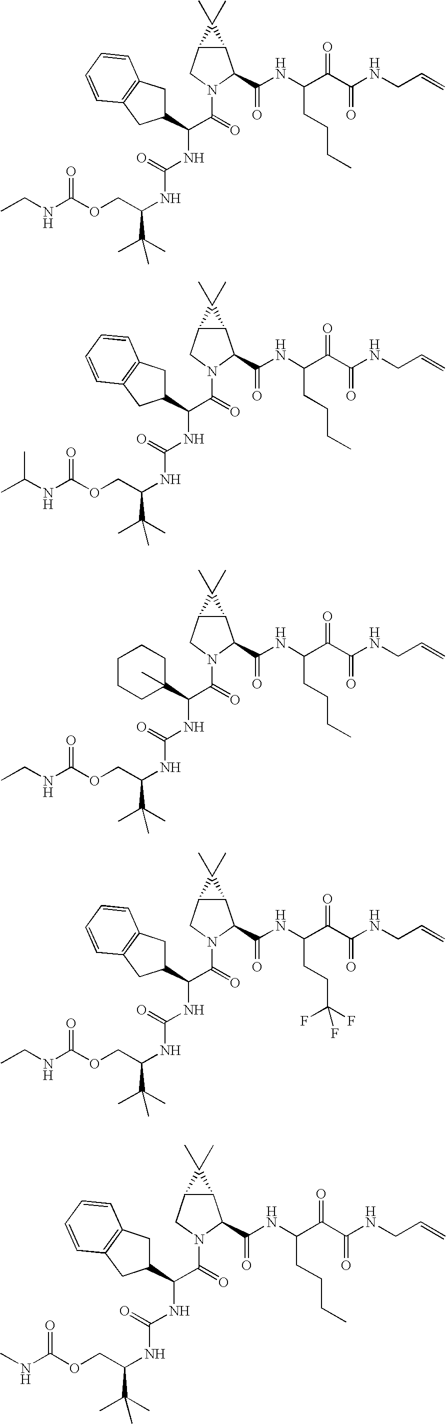 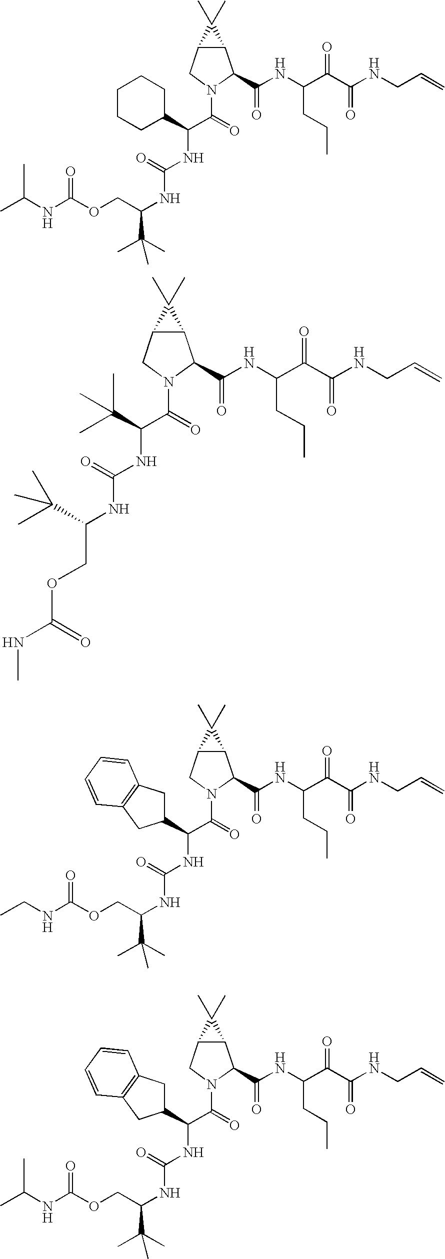 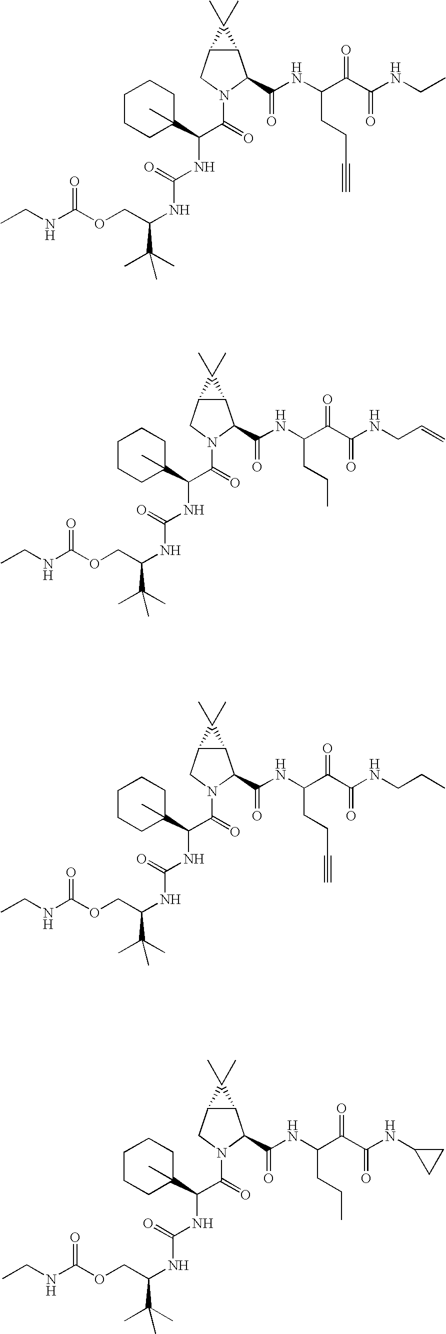 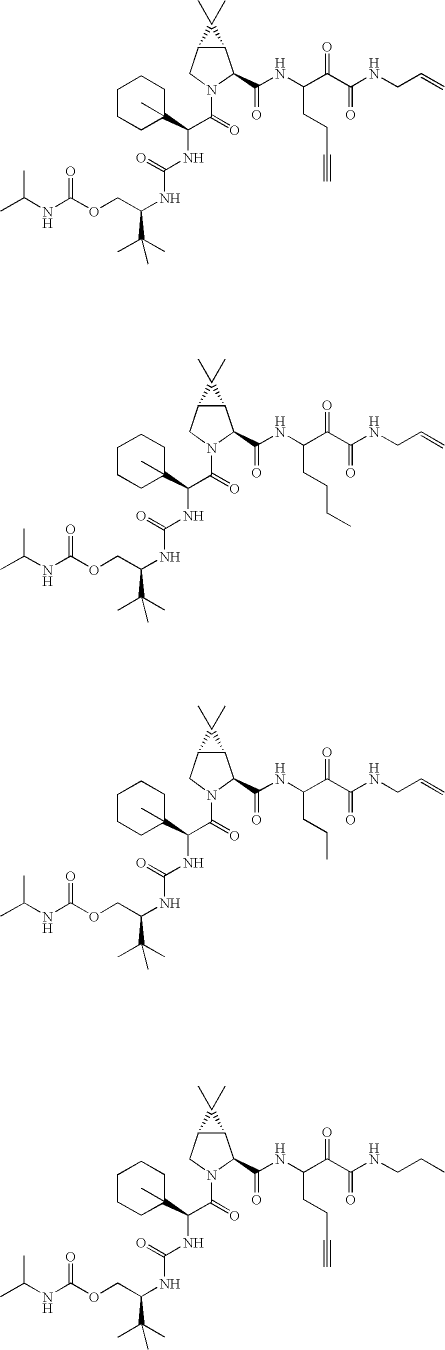 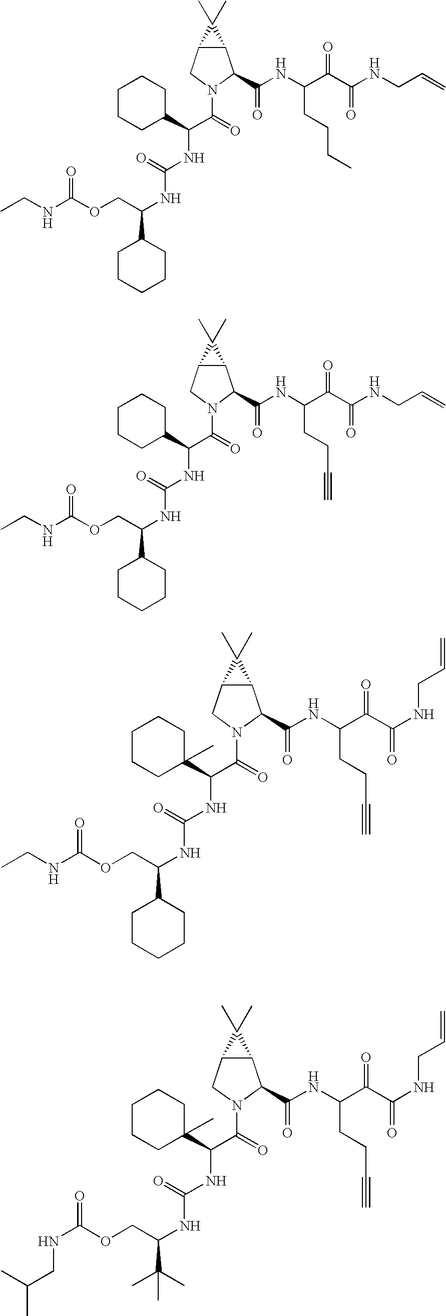 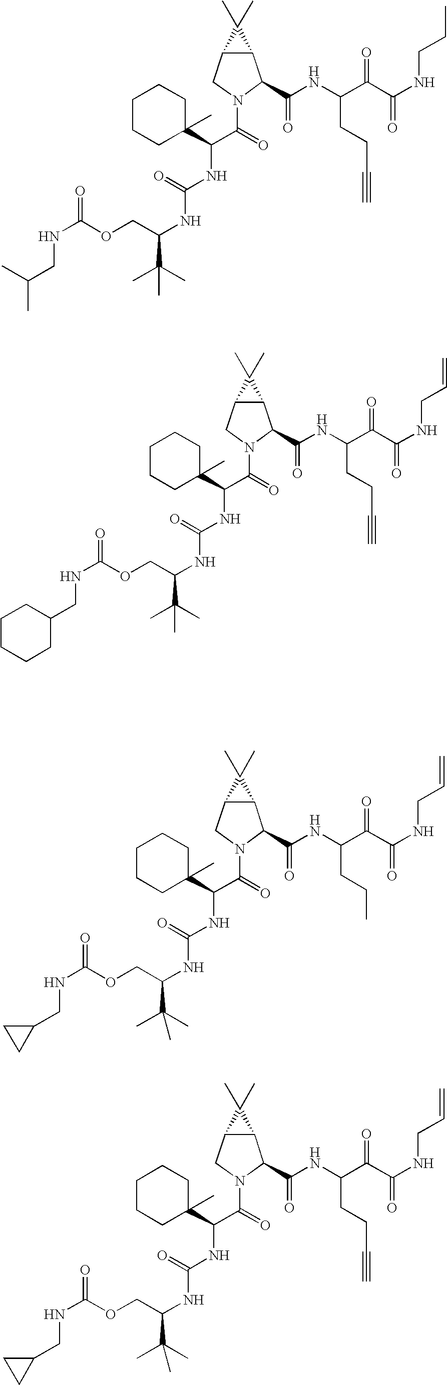 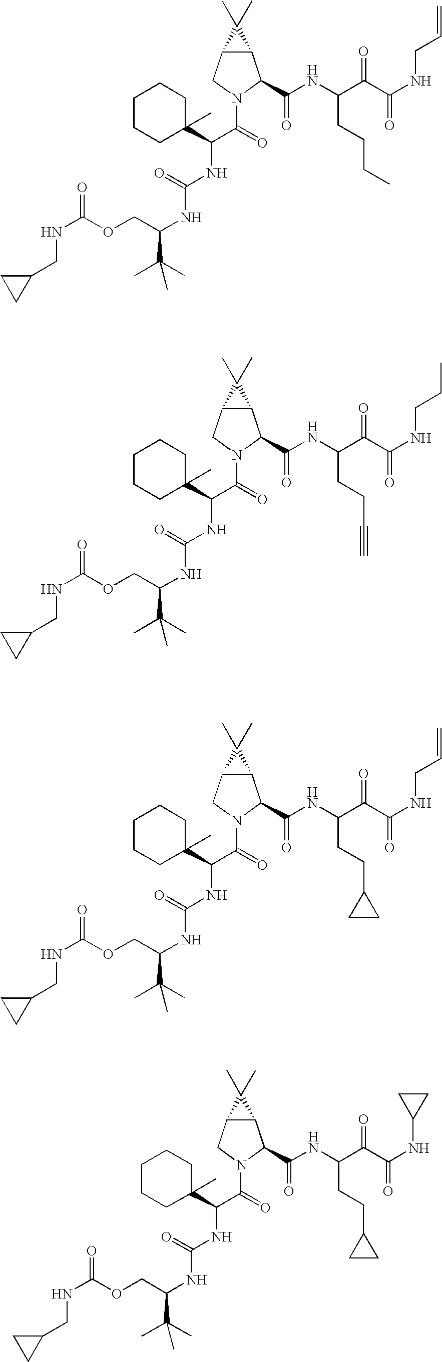
or a pharmaceutically acceptable salt, solvate or ester thereof.

Compounds of formula XIII are disclosed in U.S. Patent Application Publication US 2005/0222047 published Oct. 6, 2005, which is incorporated herein by reference in its entirety for all purposes.

Non-limiting examples of certain compounds disclosed in US 2005/0222047 are: 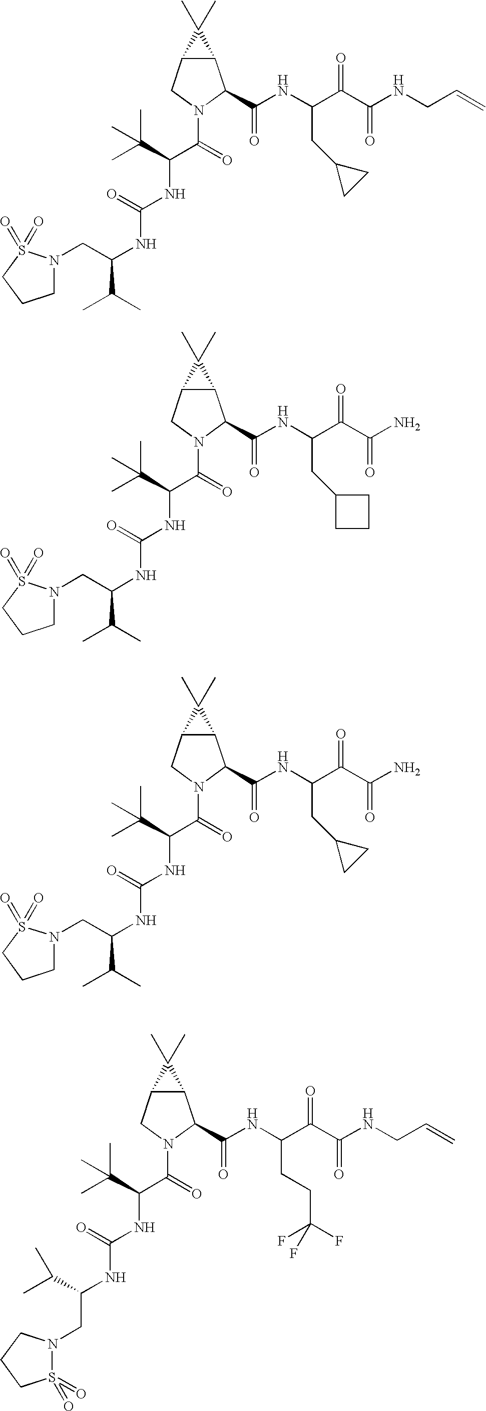 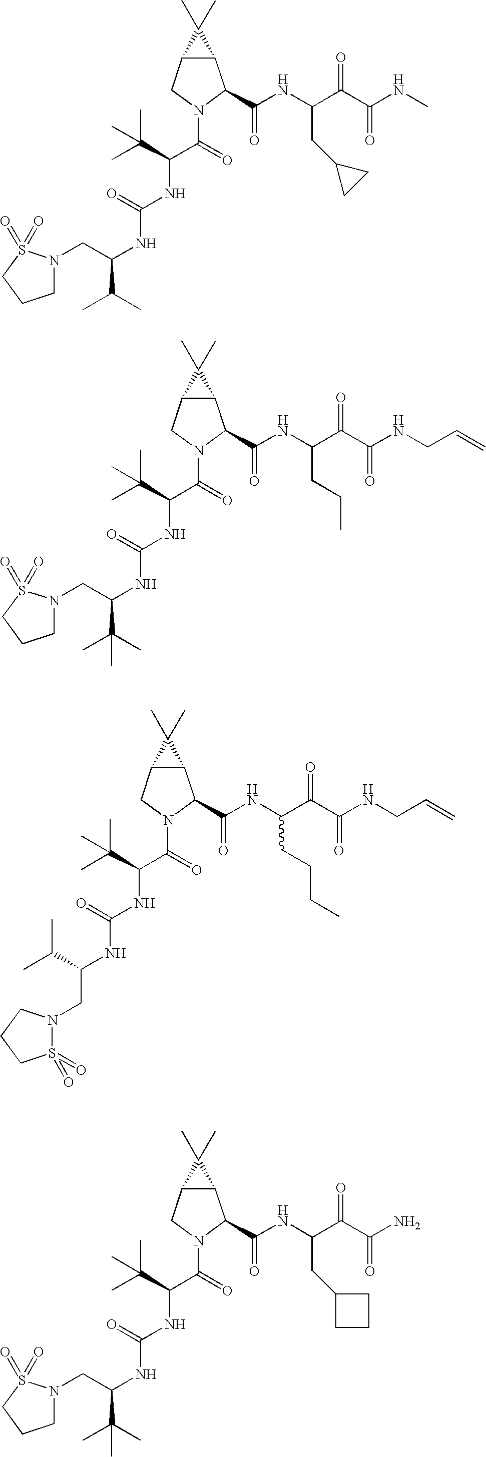 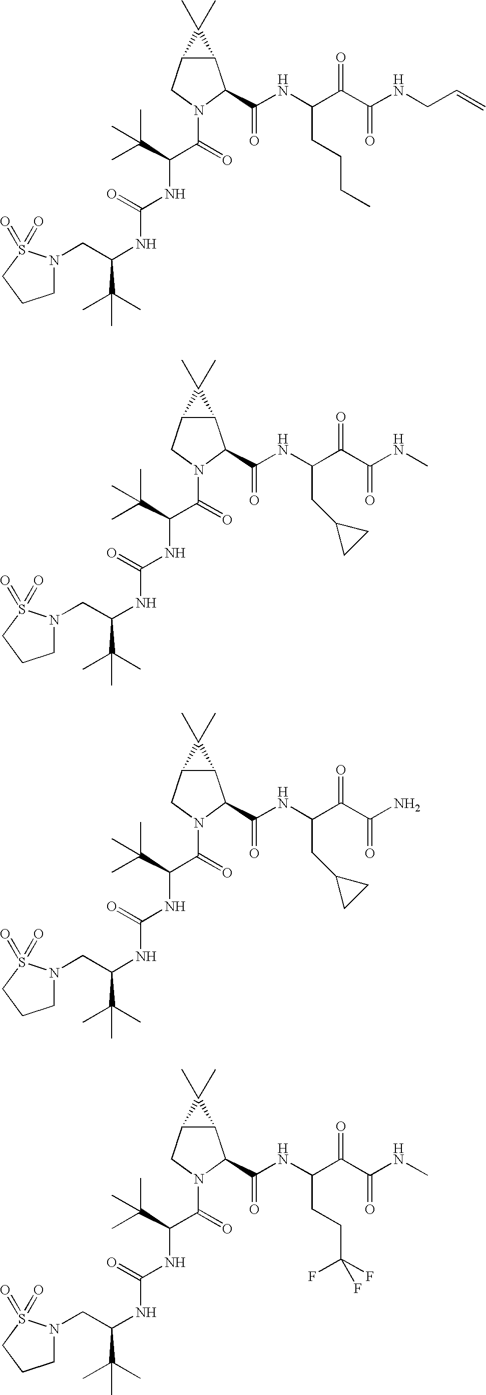 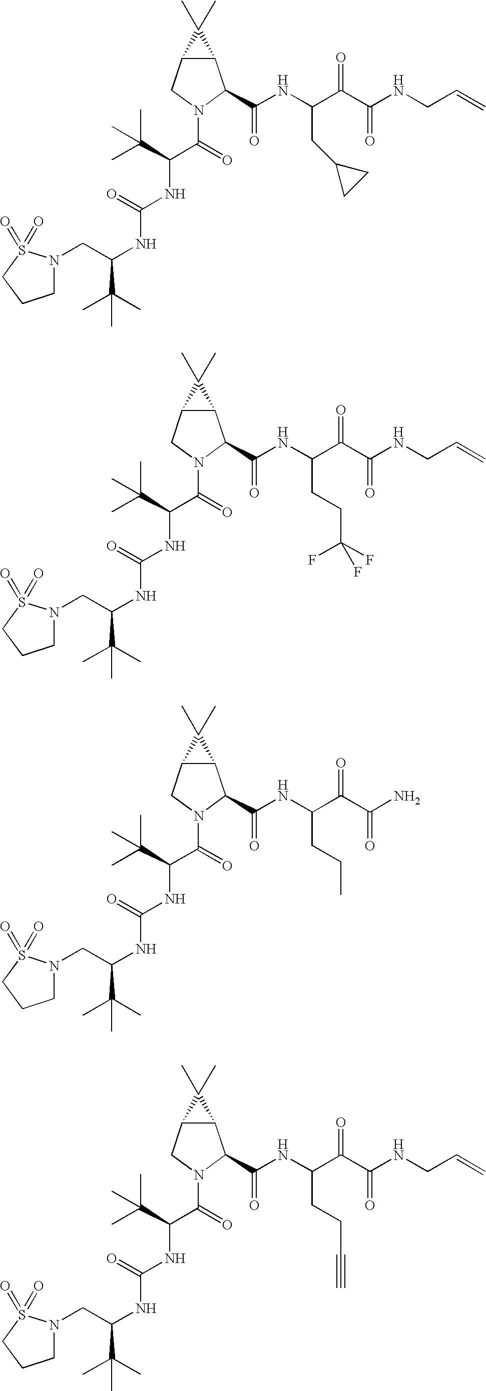 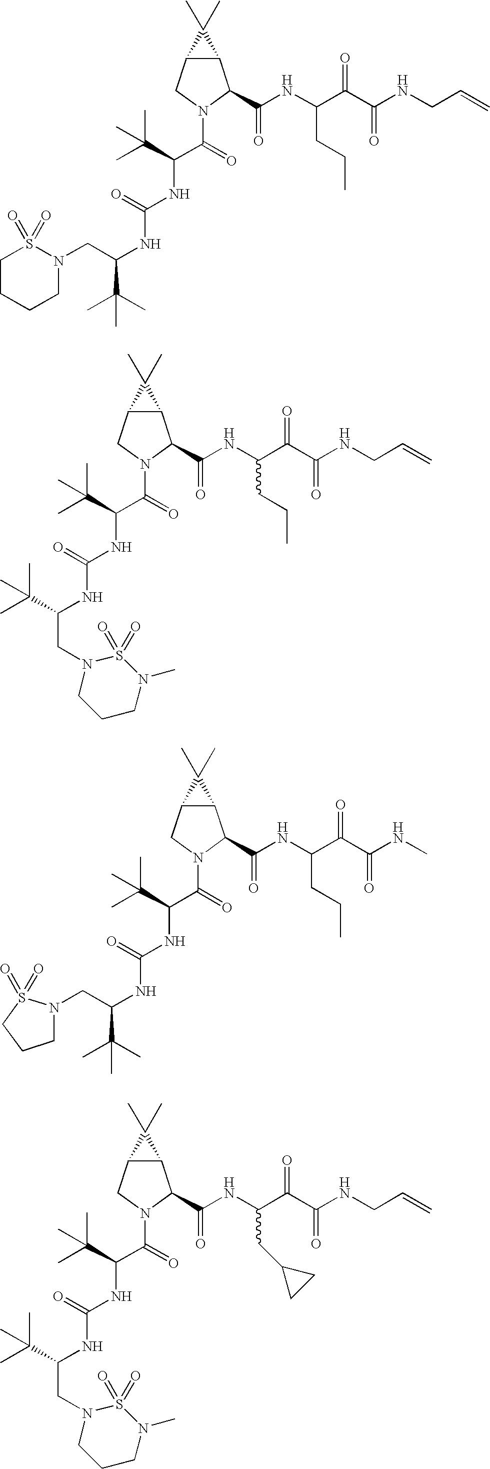 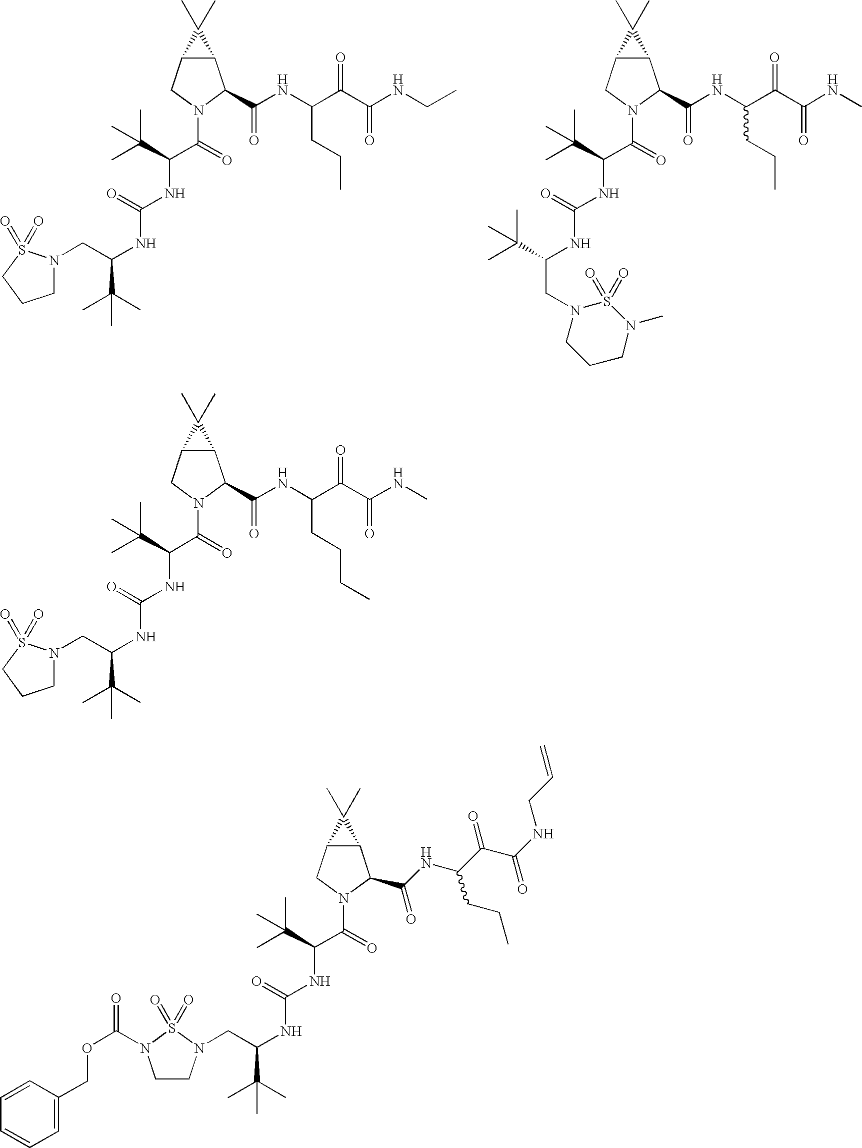 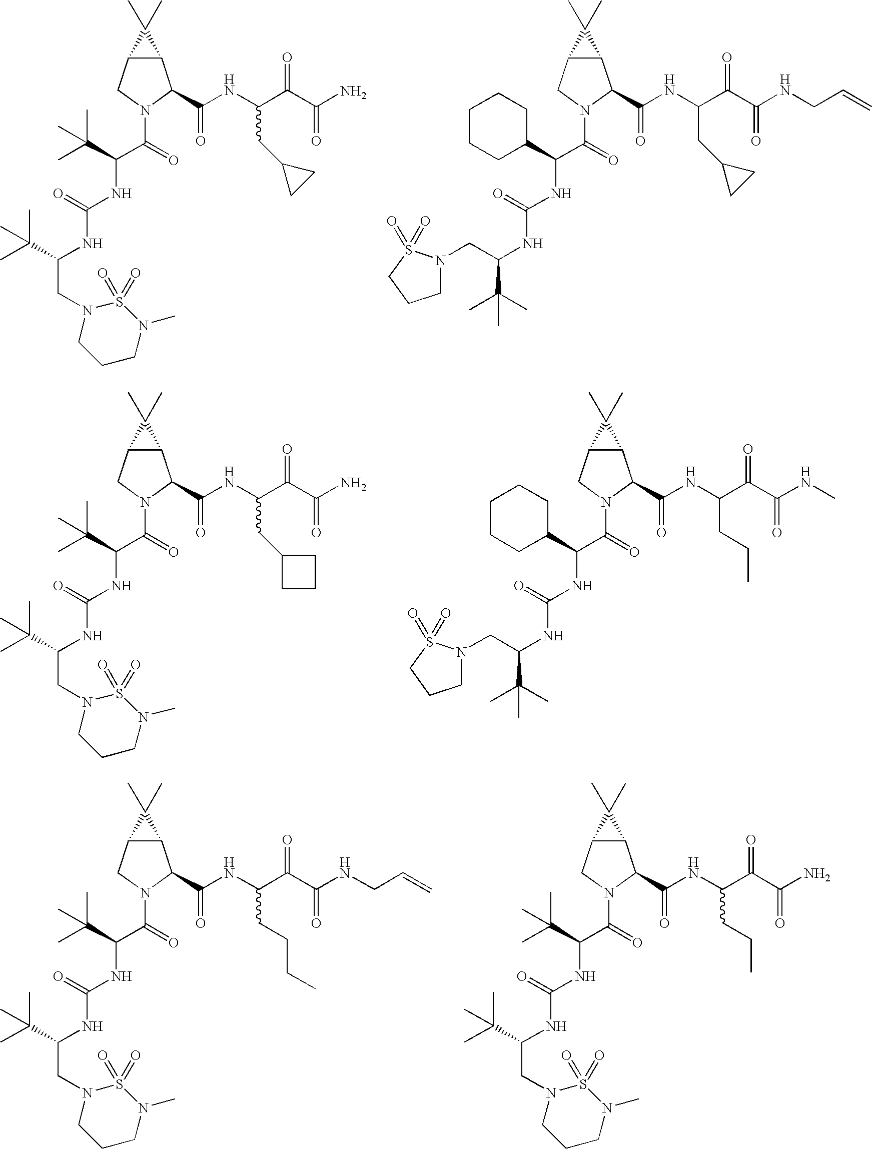 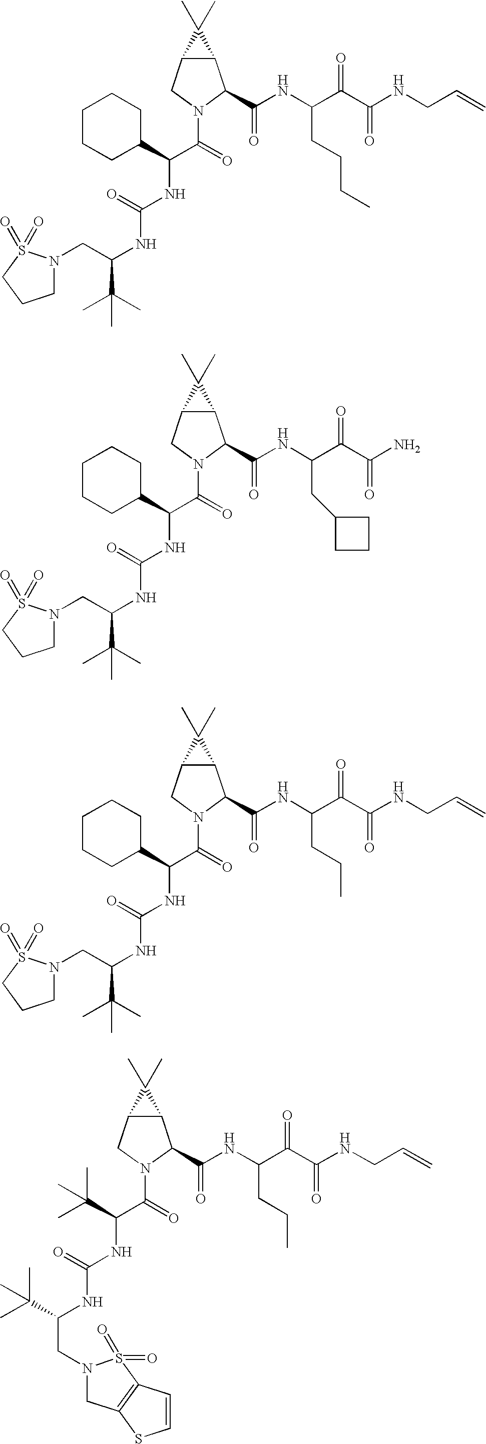 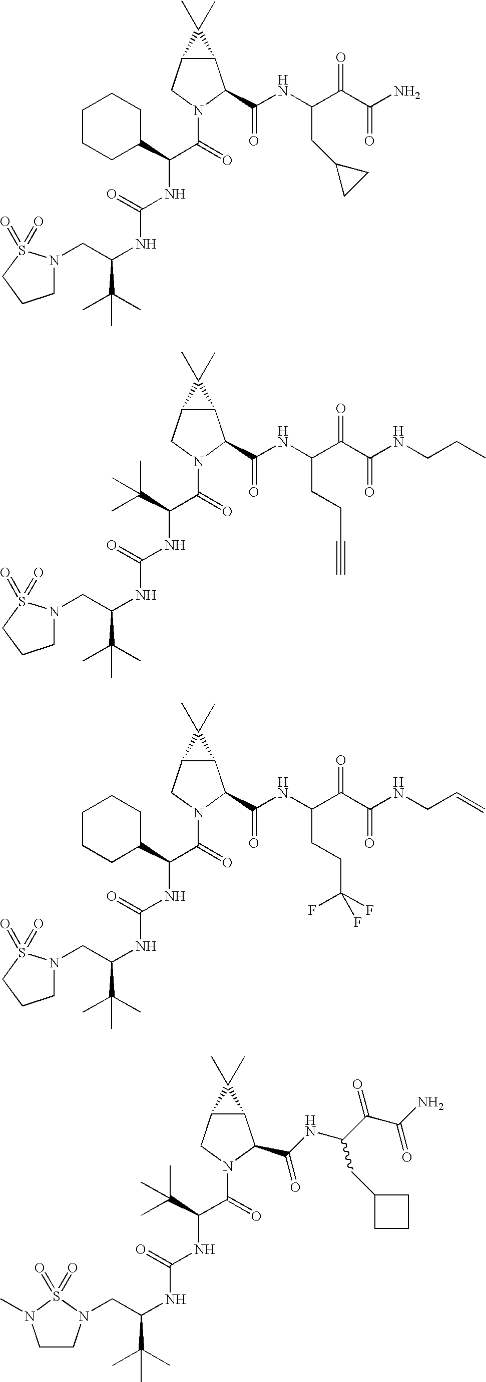 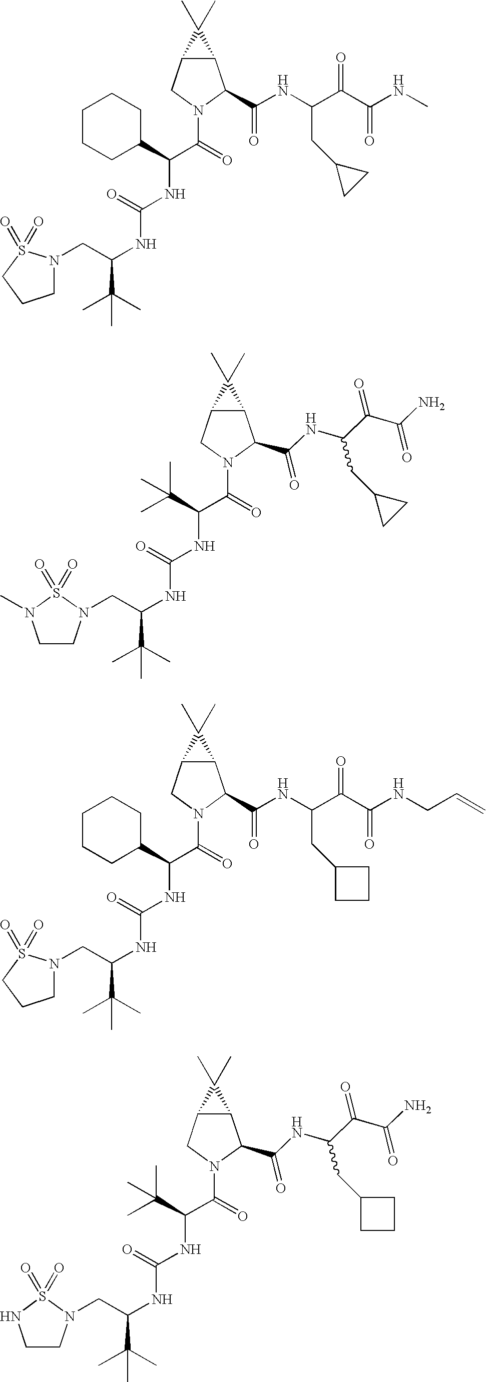 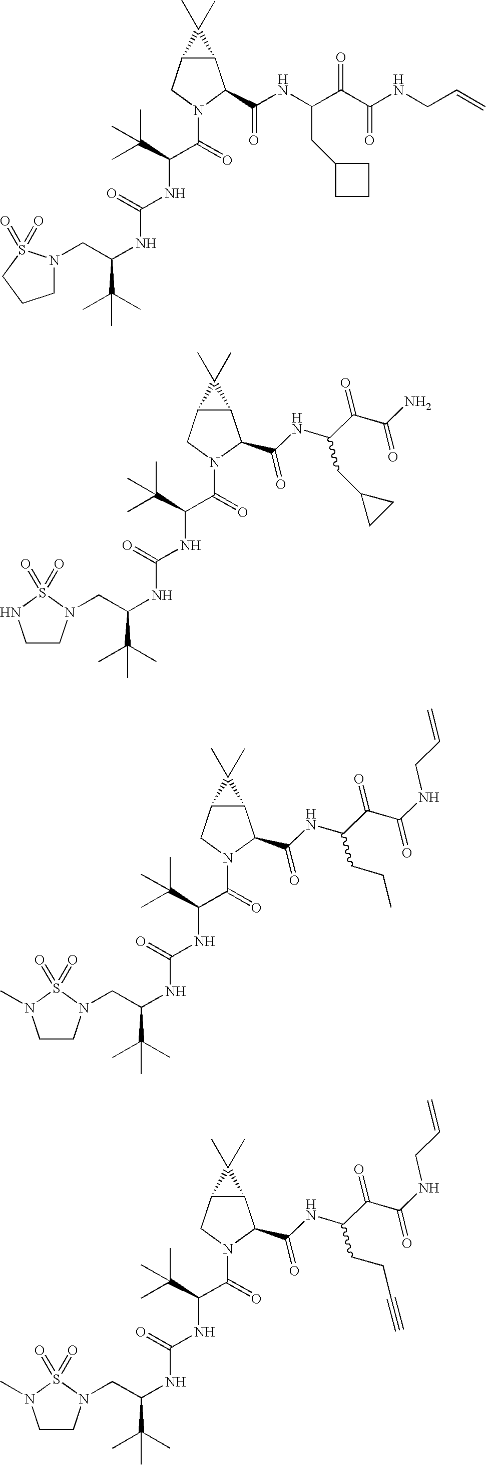 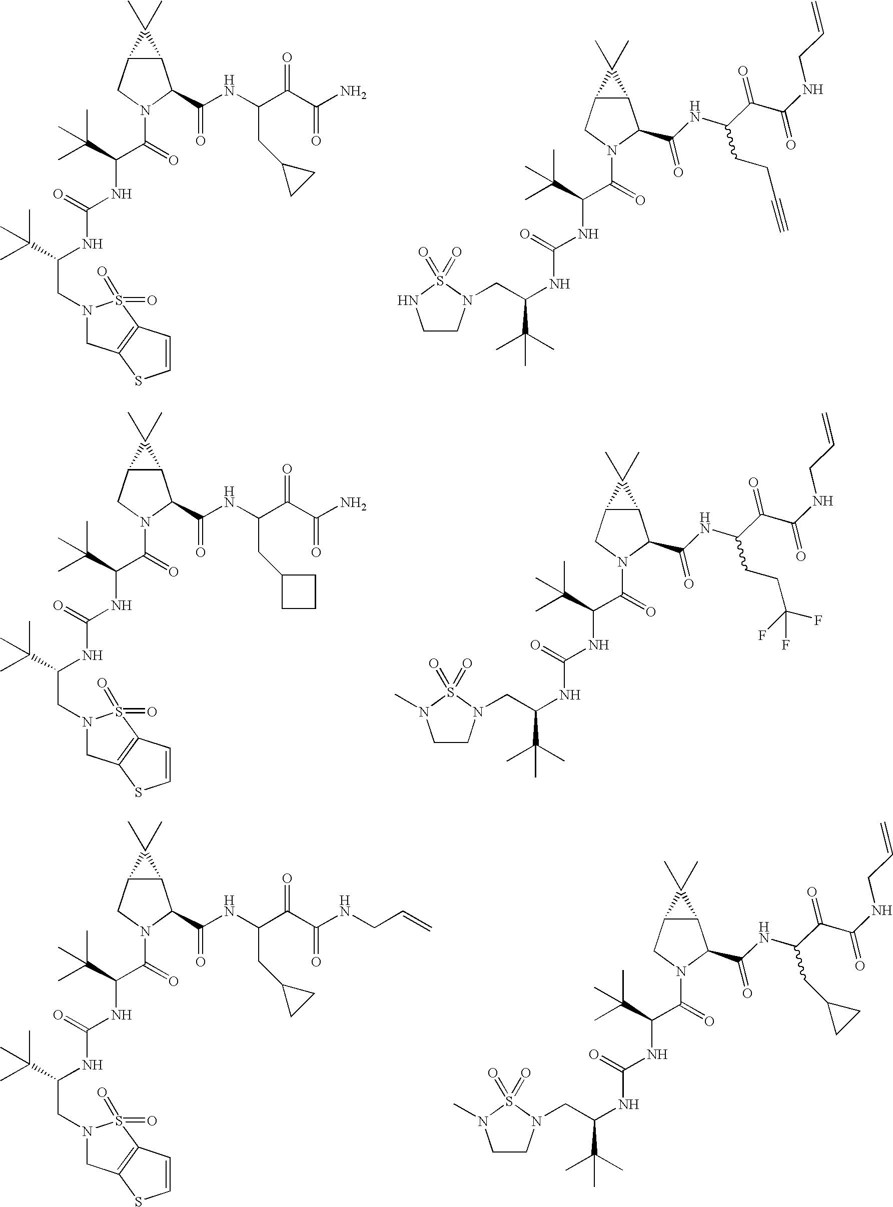 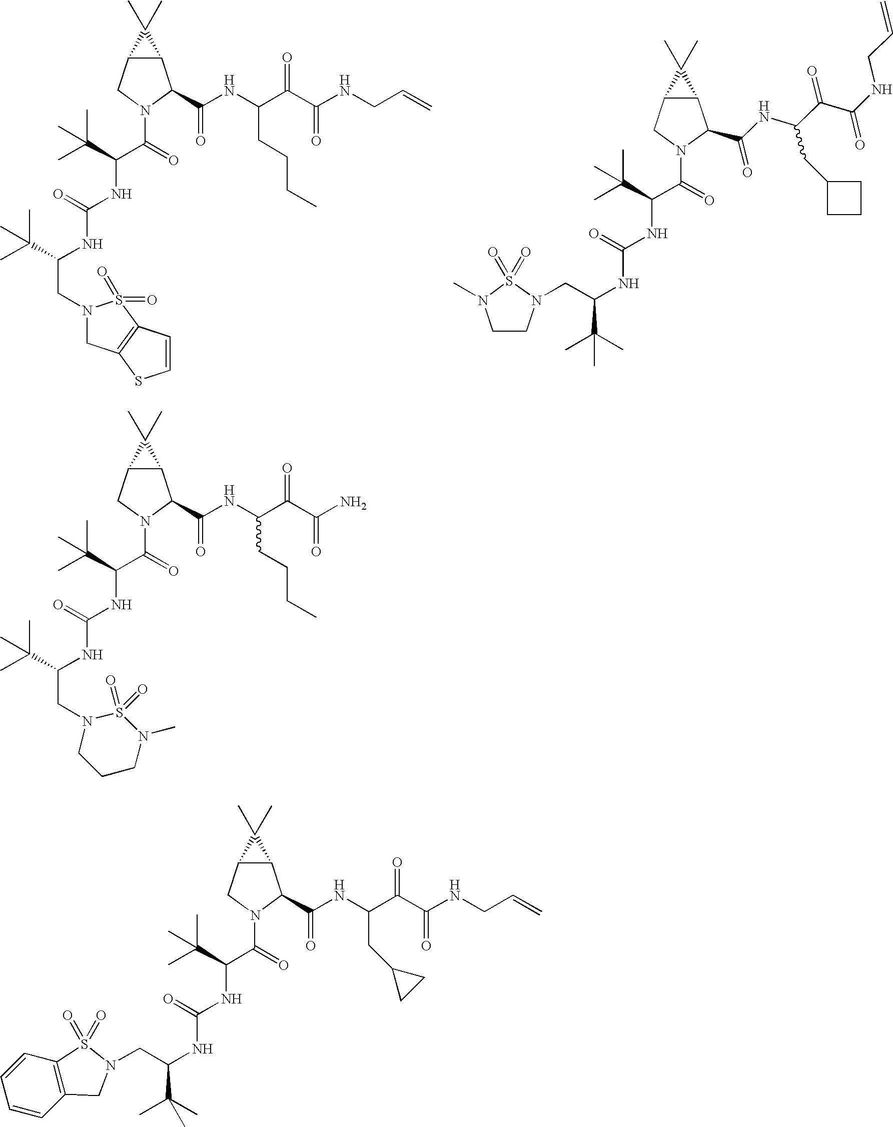 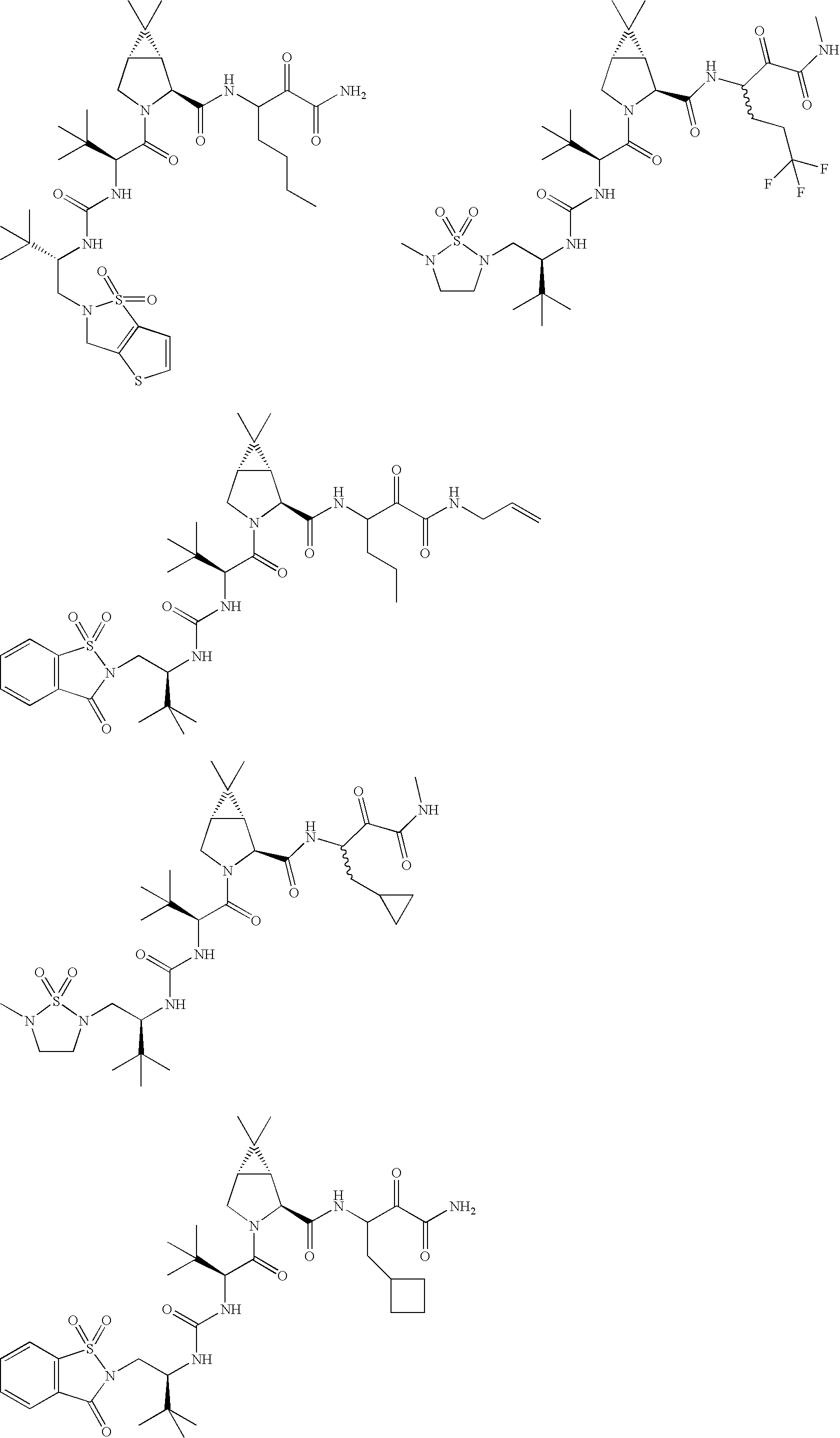 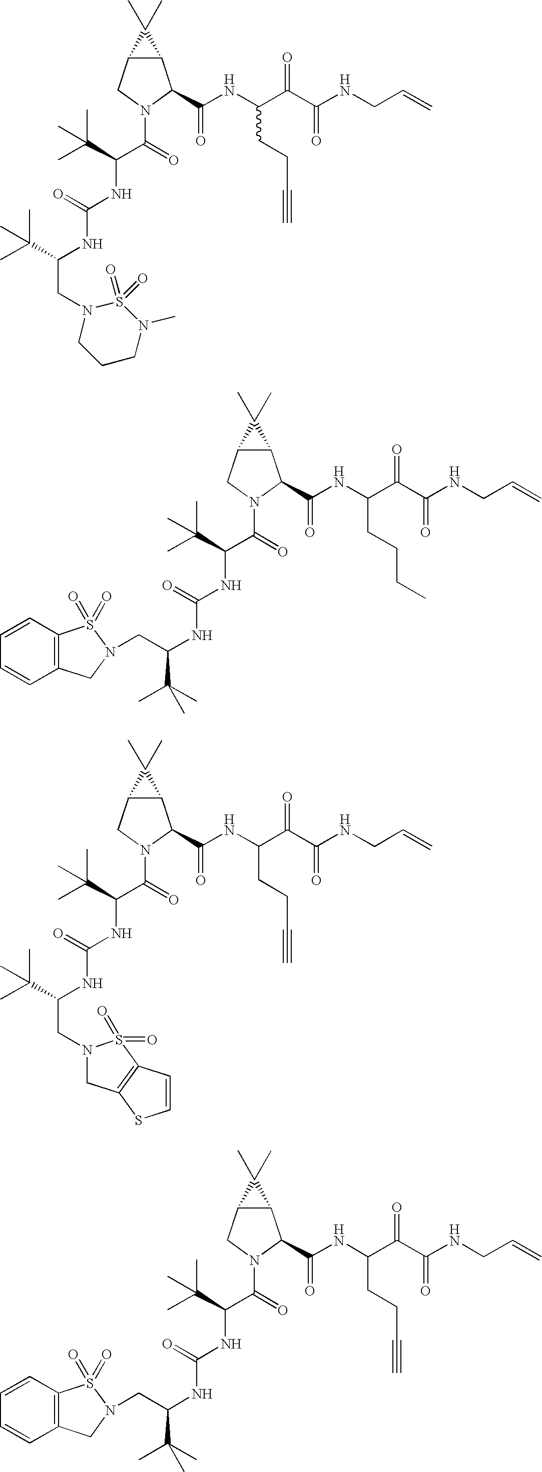 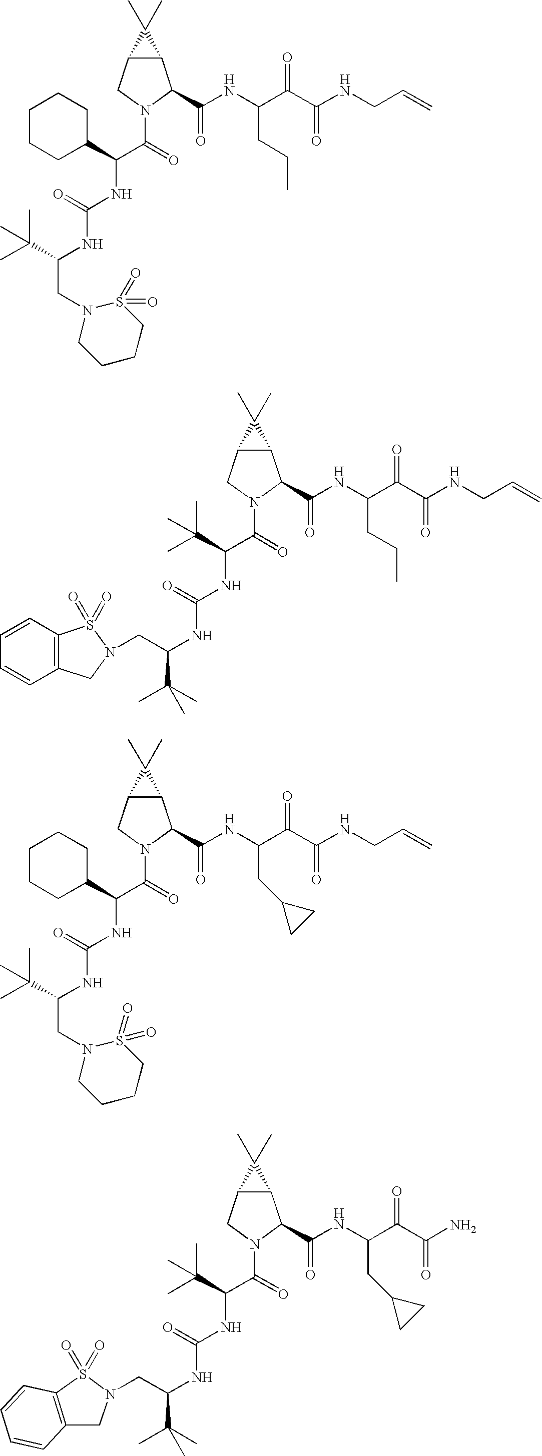 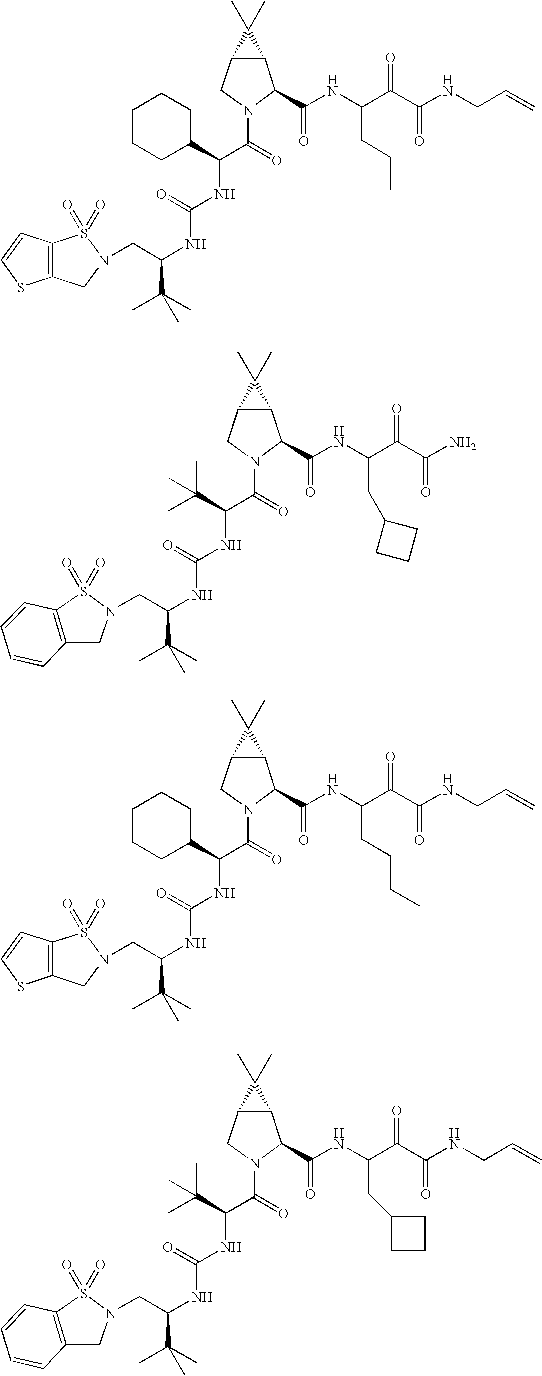 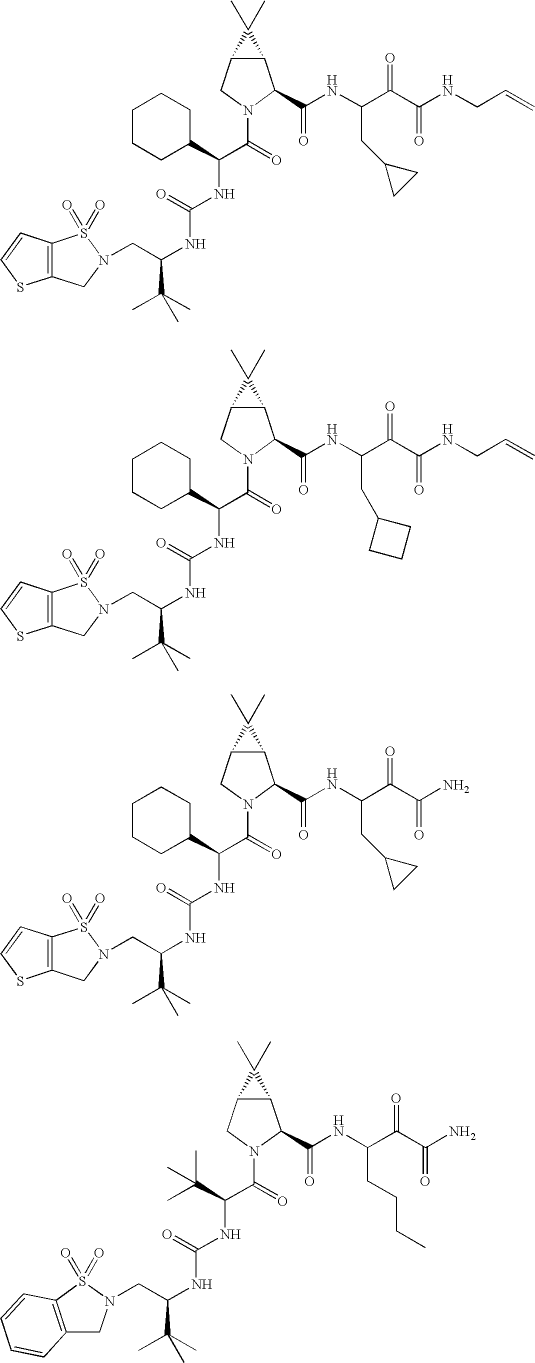 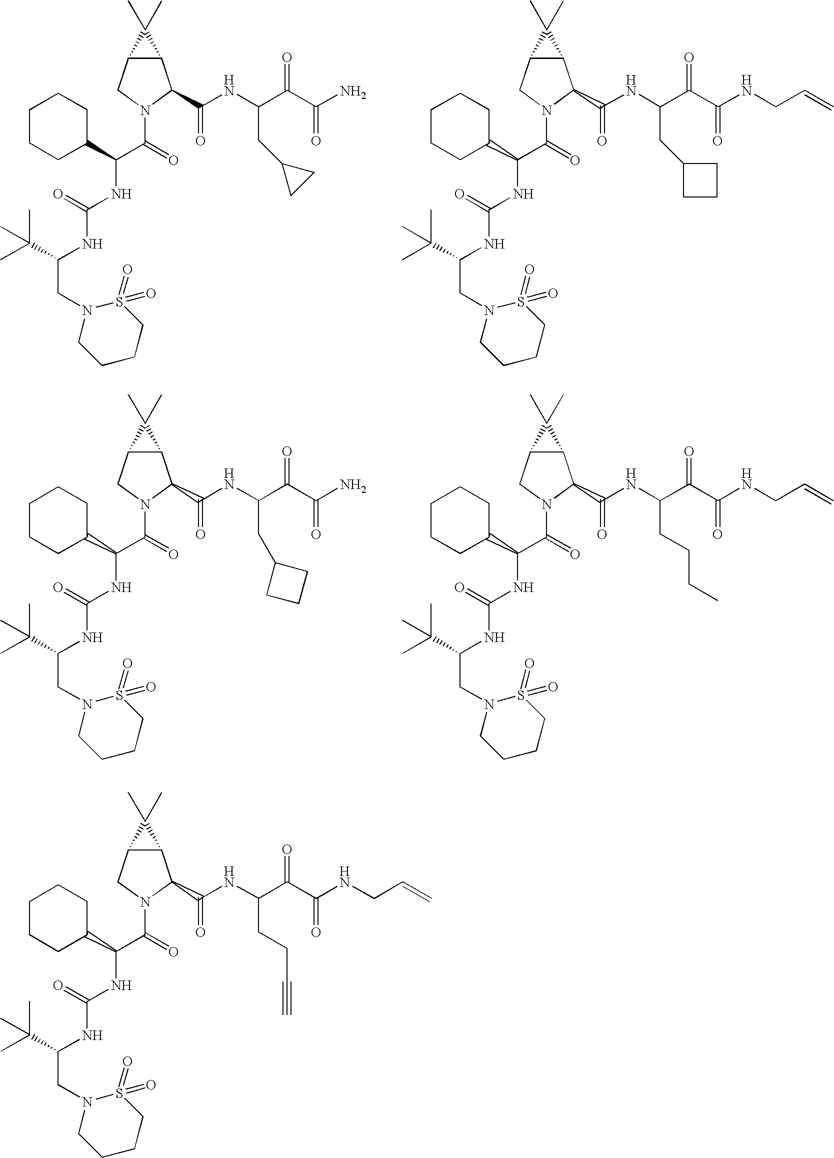 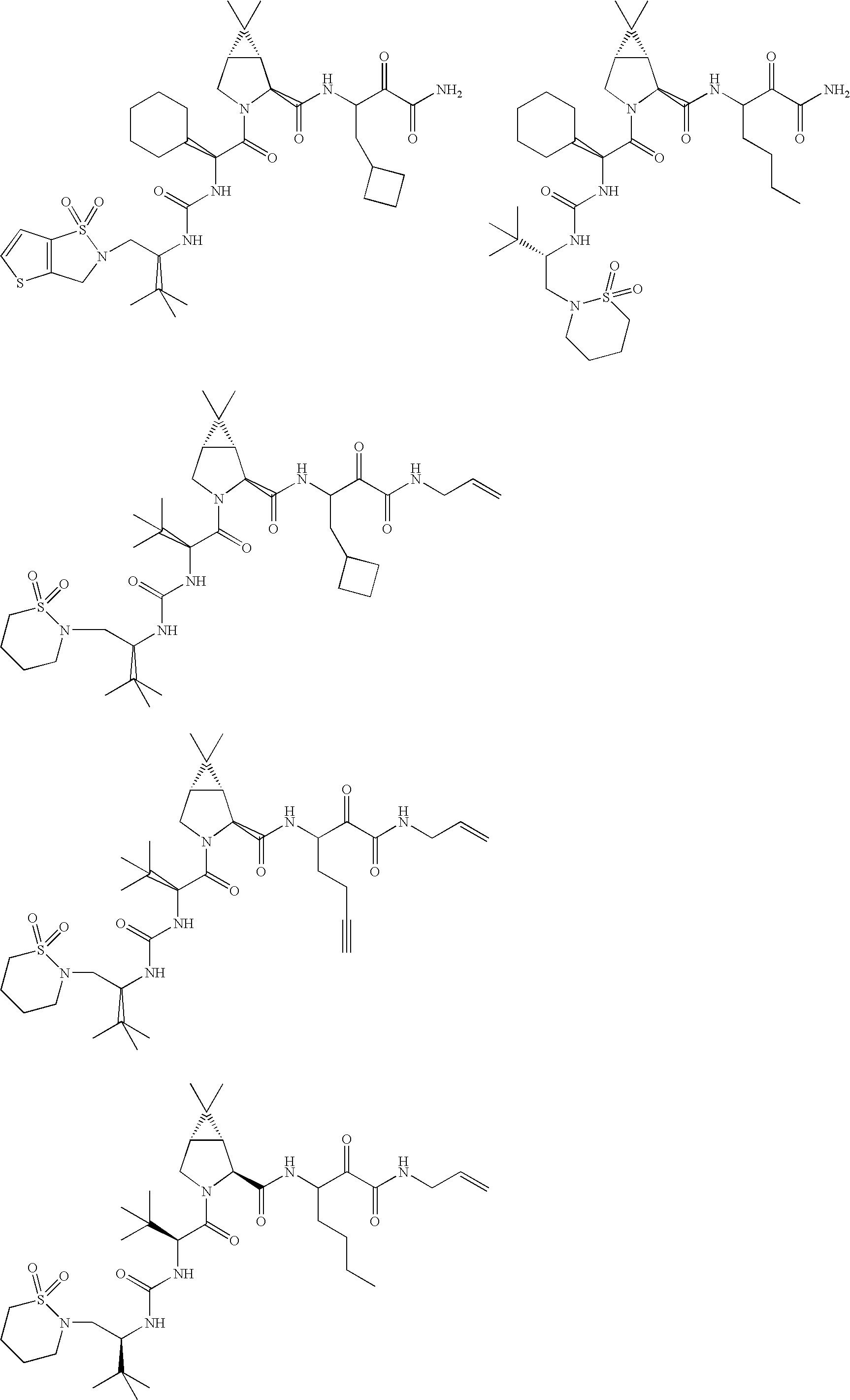 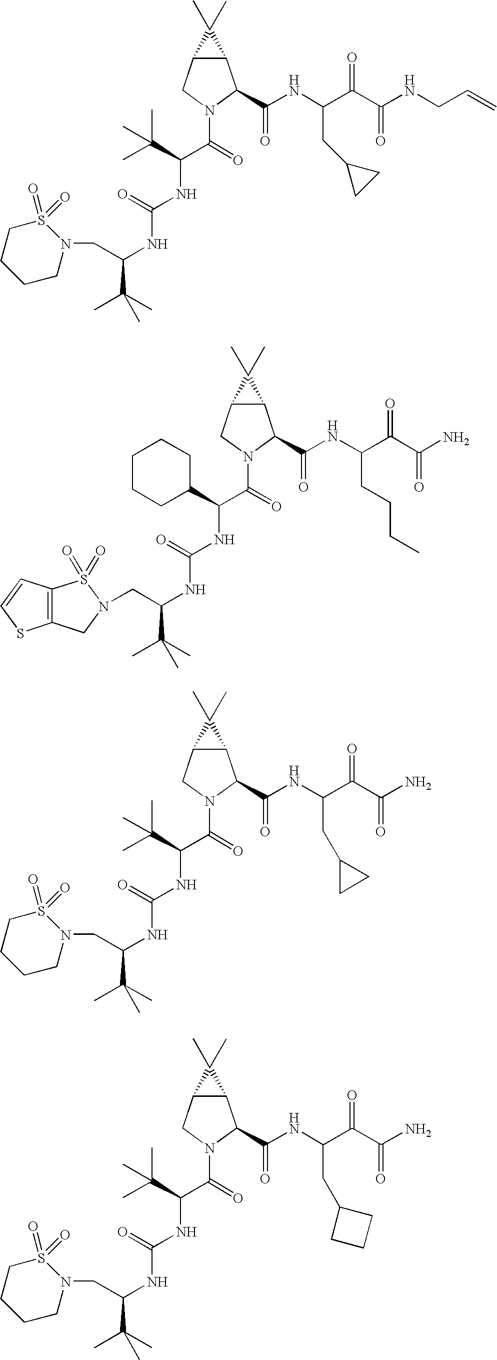 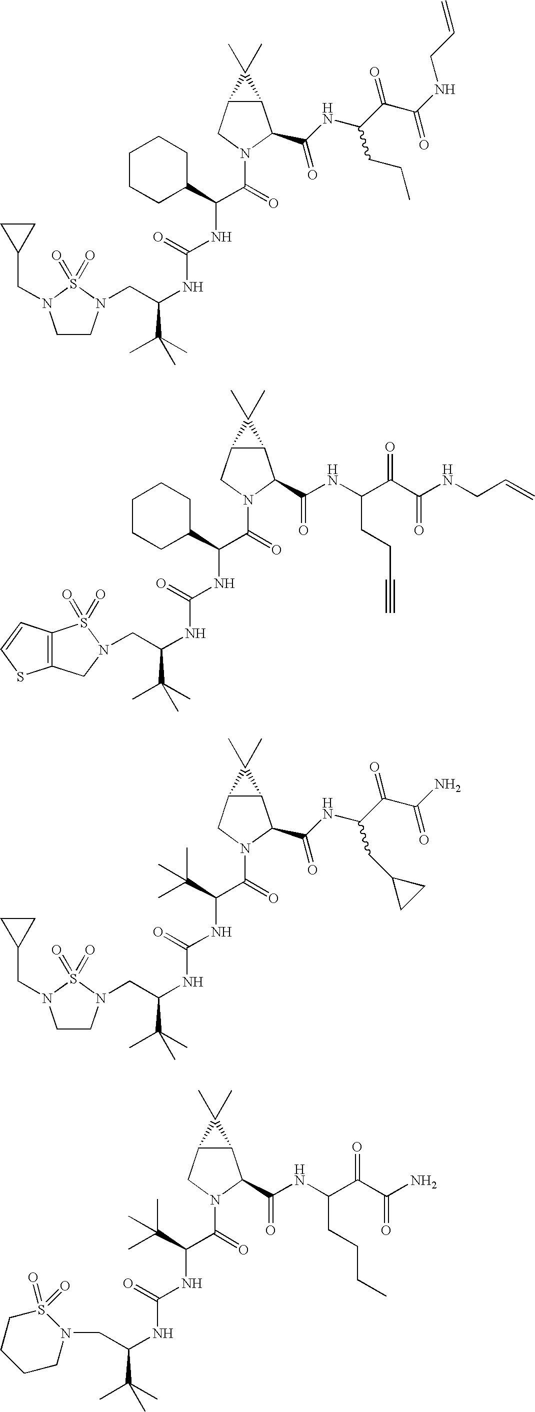 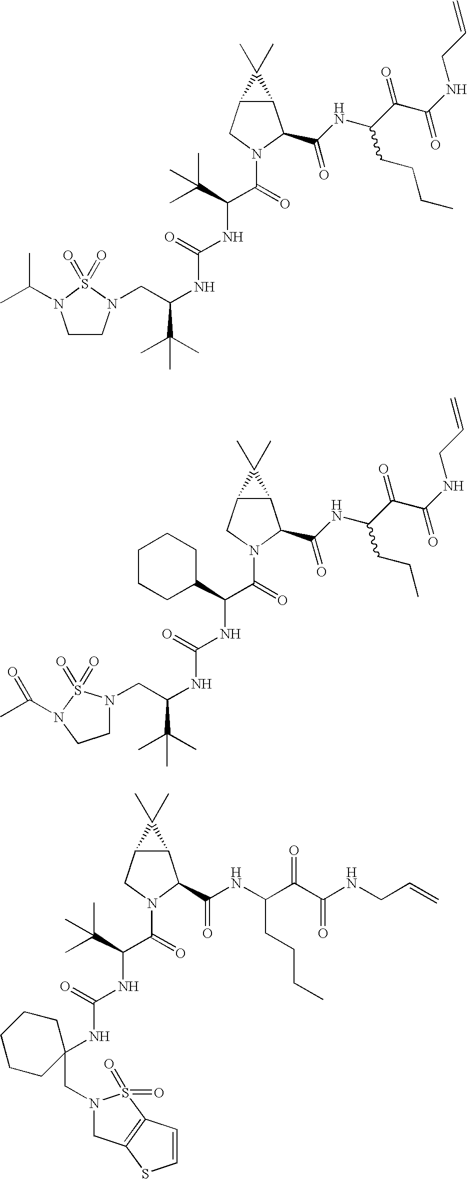 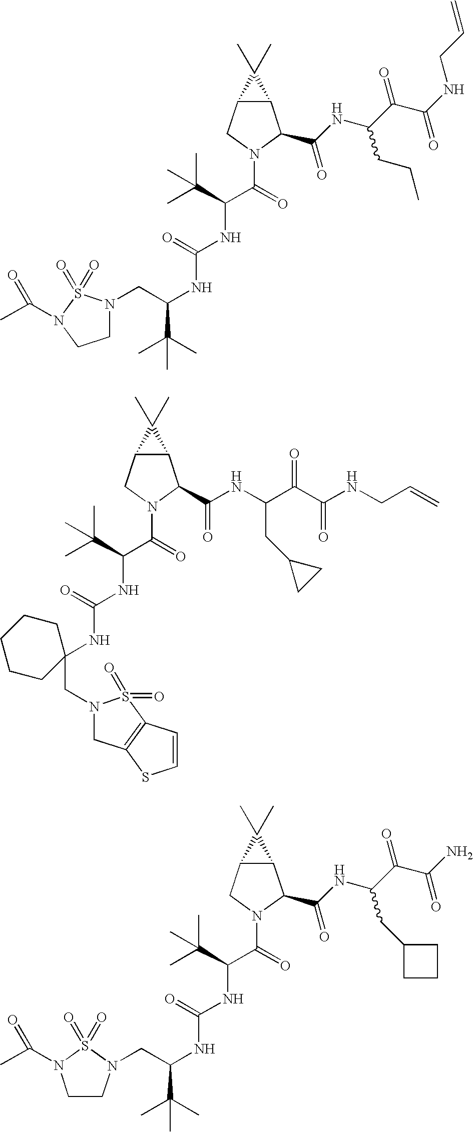 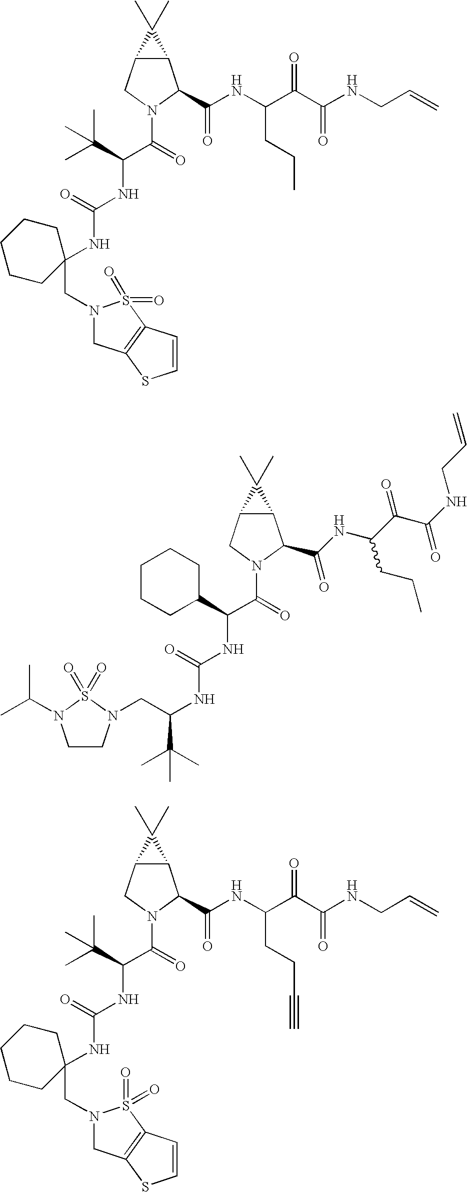 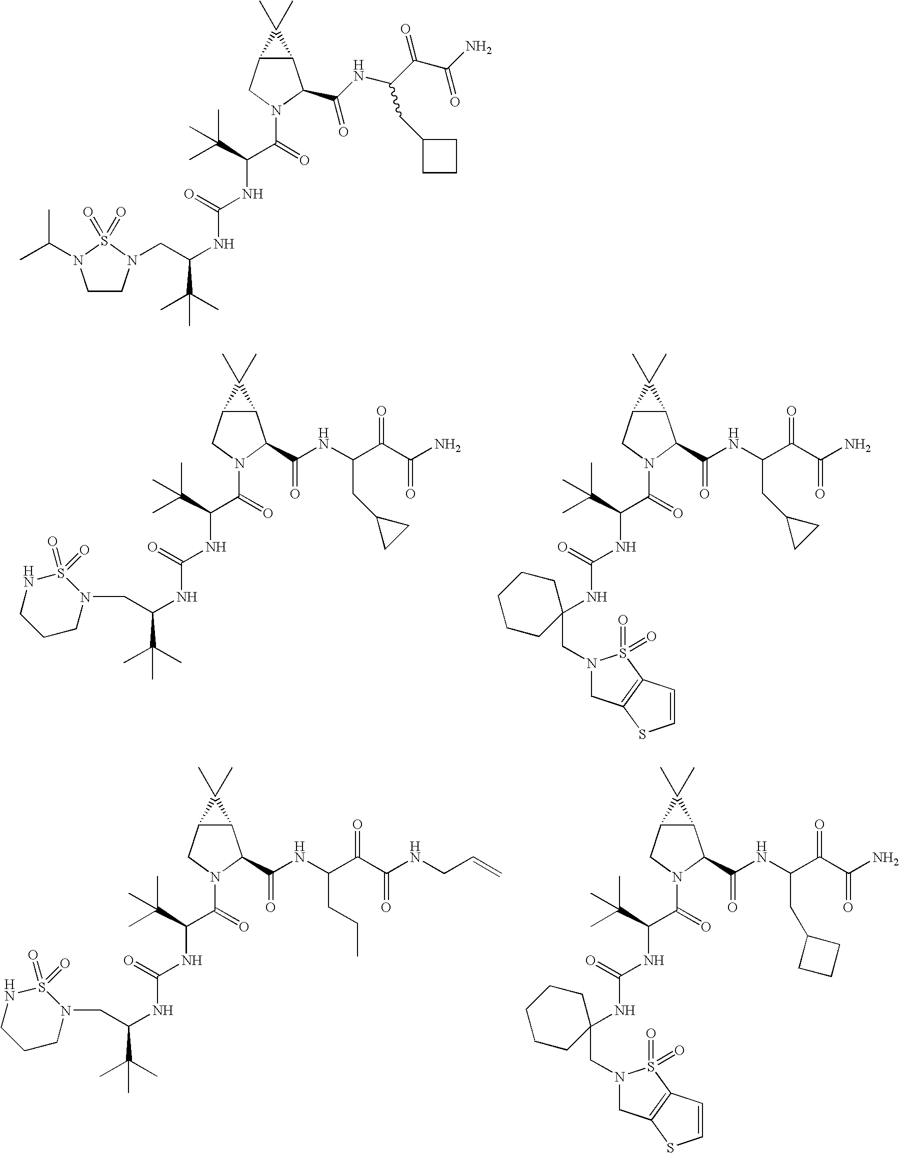 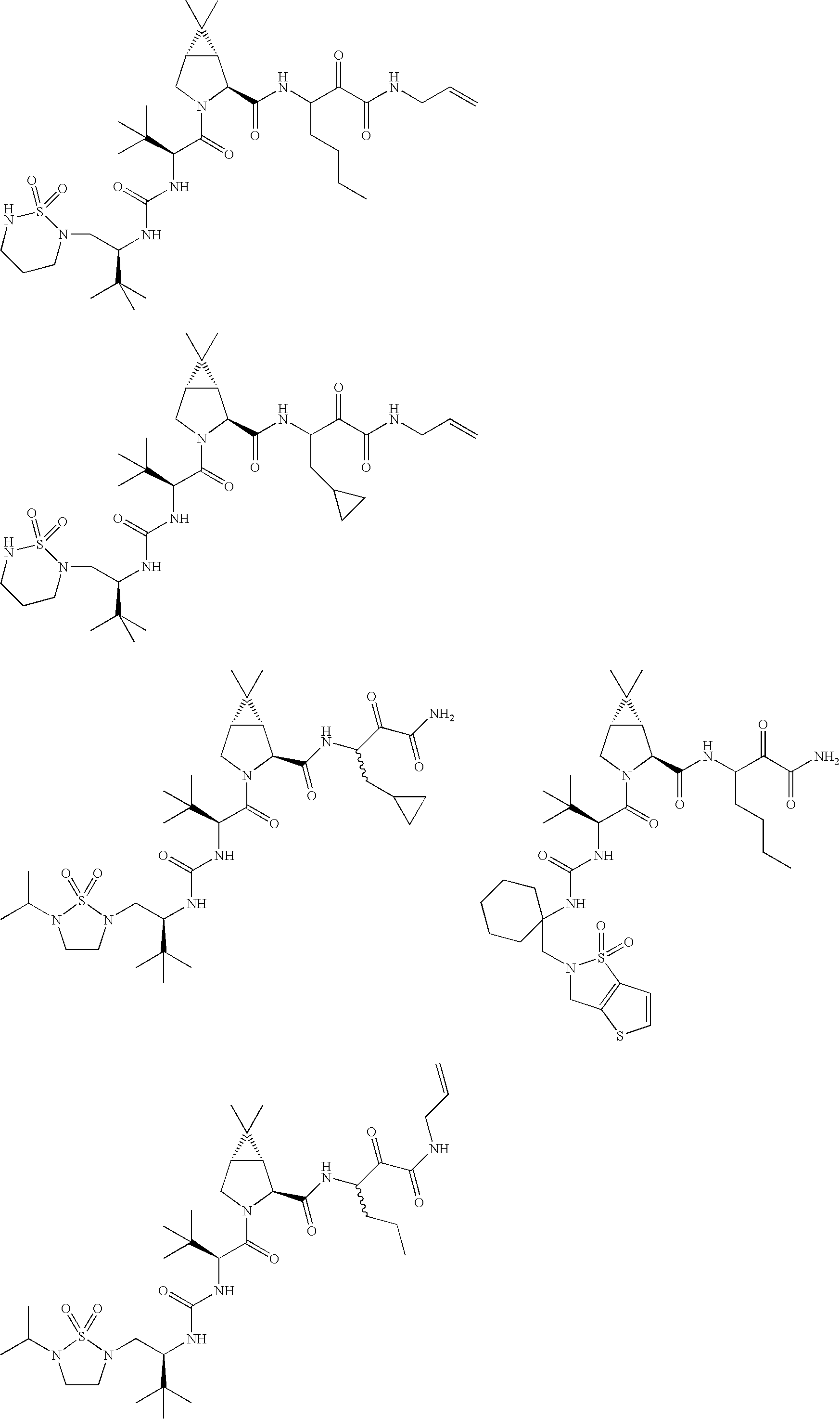 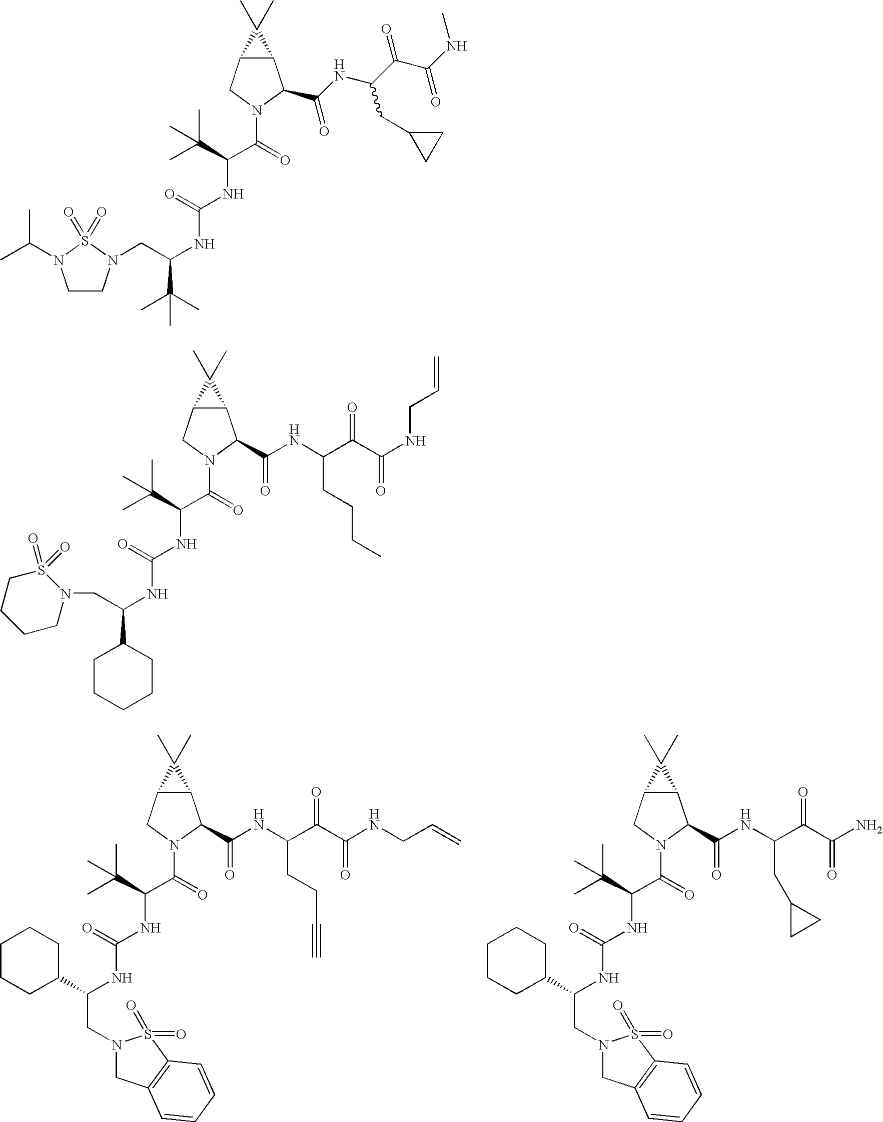 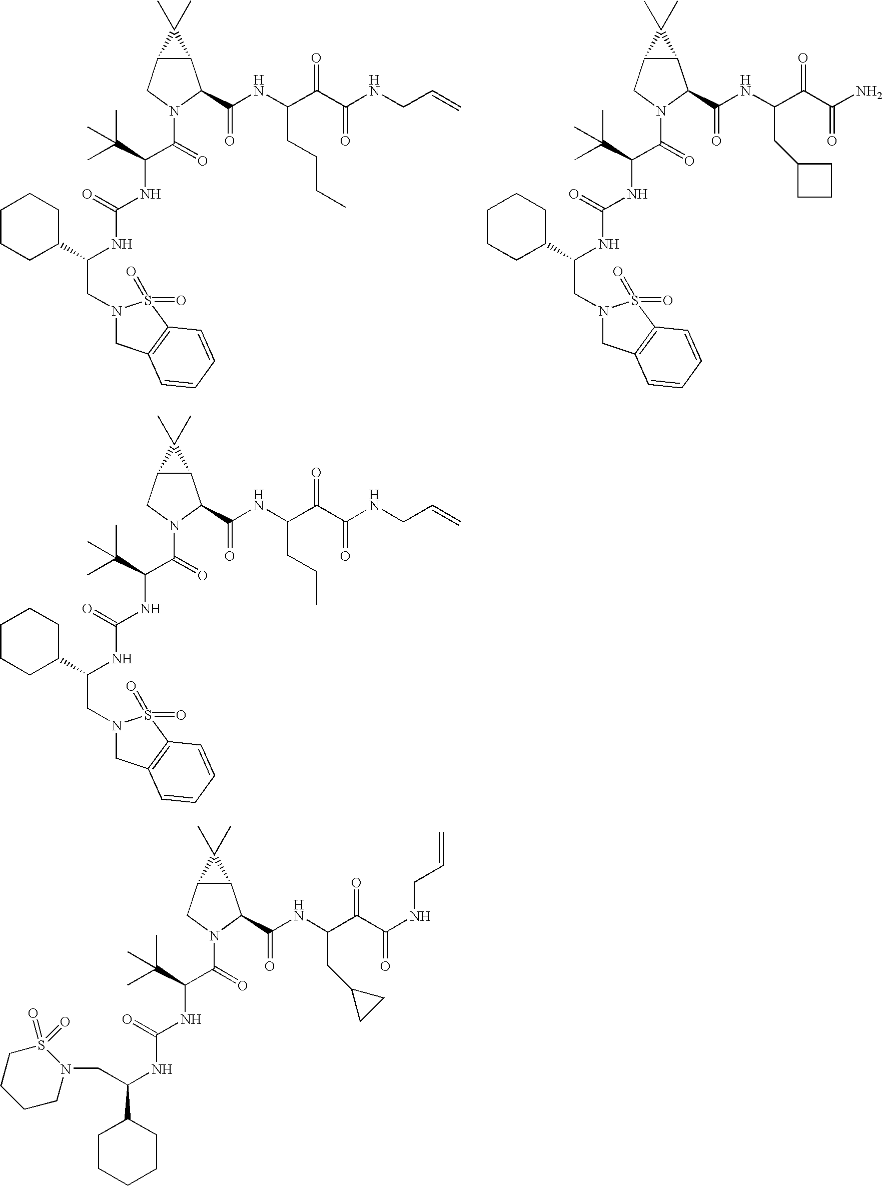 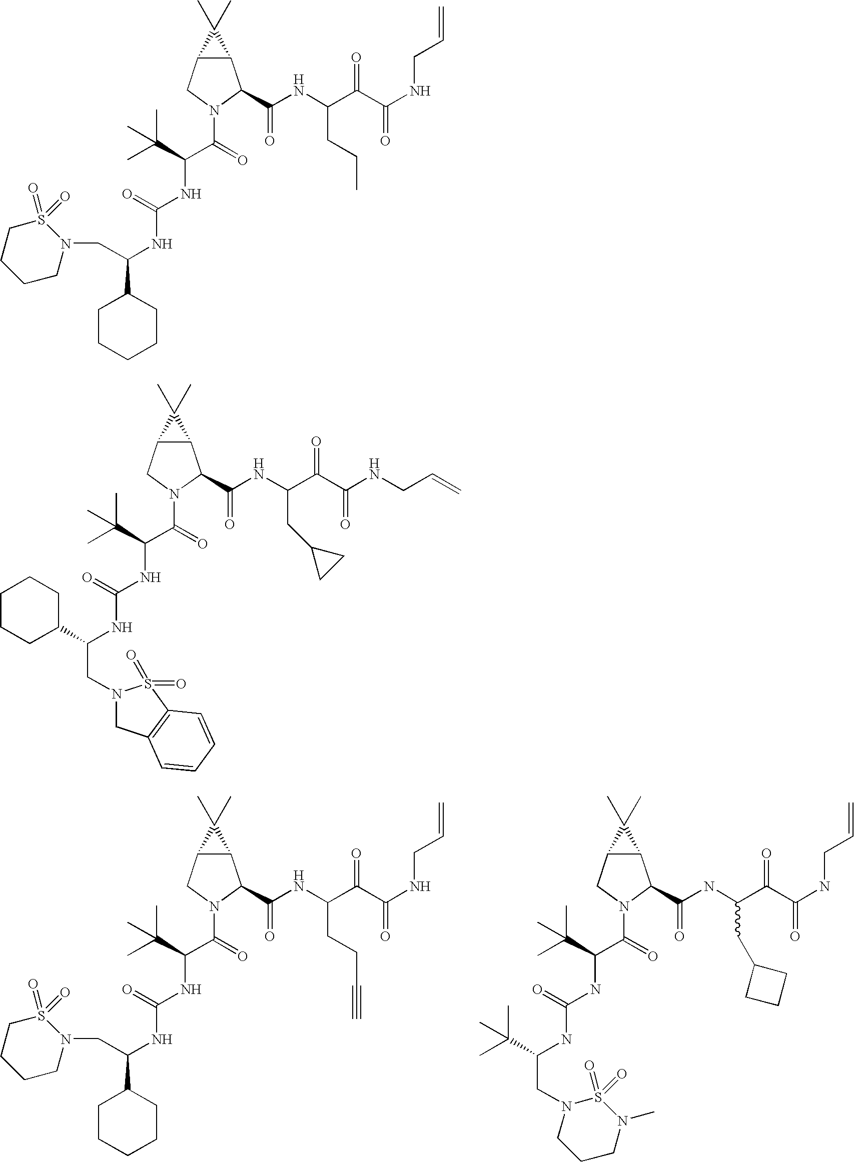 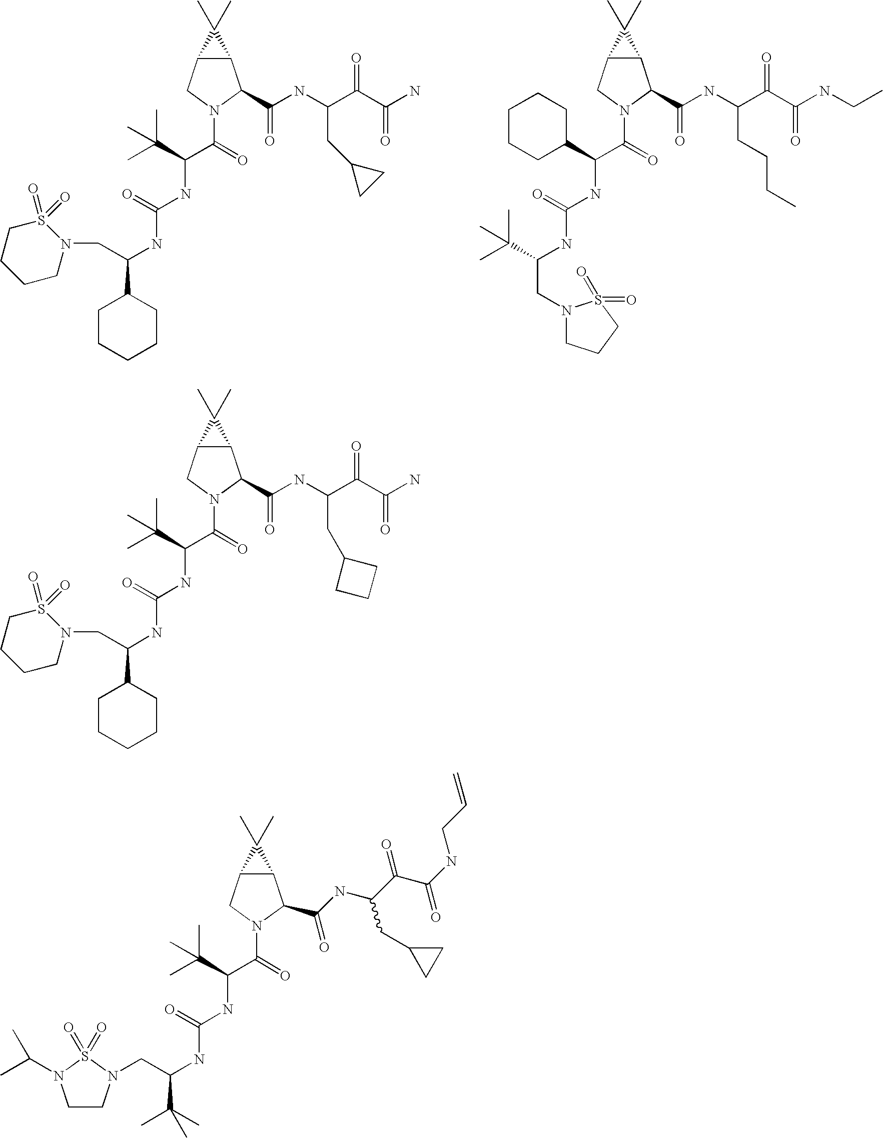 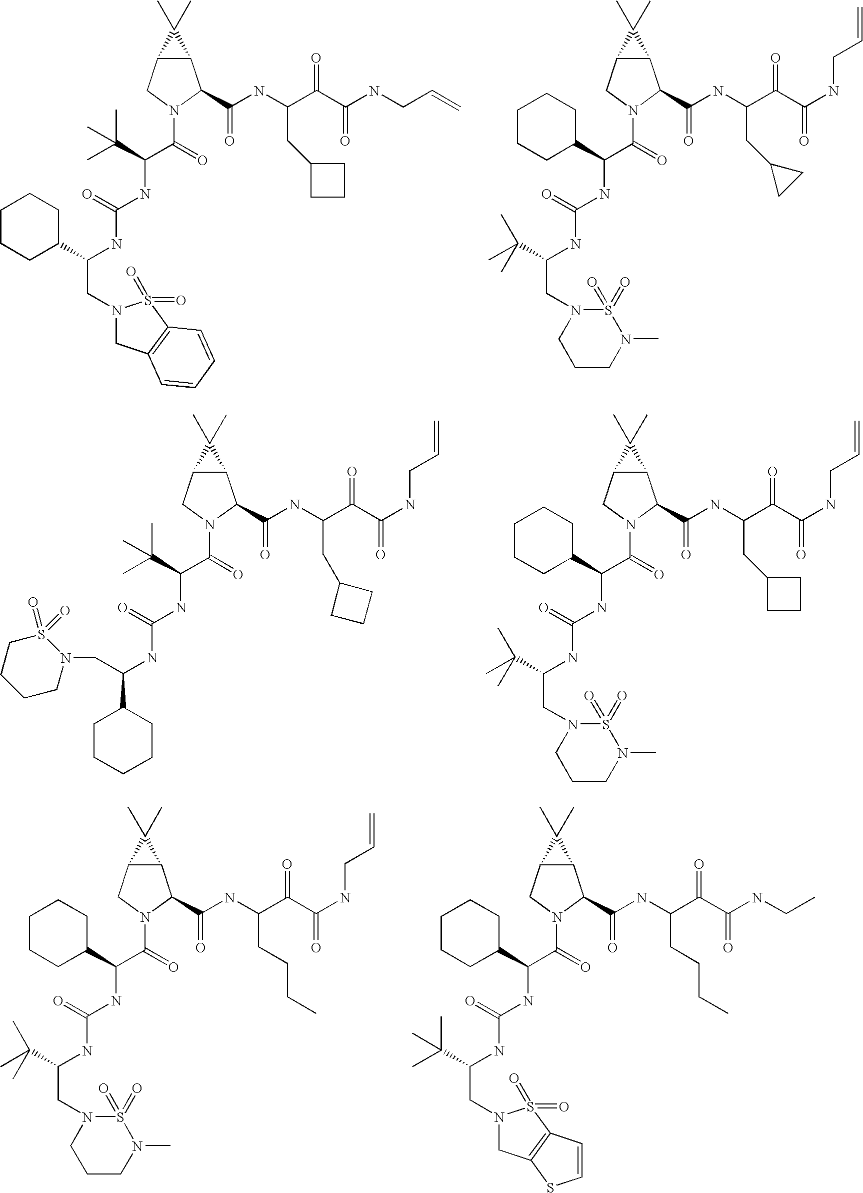 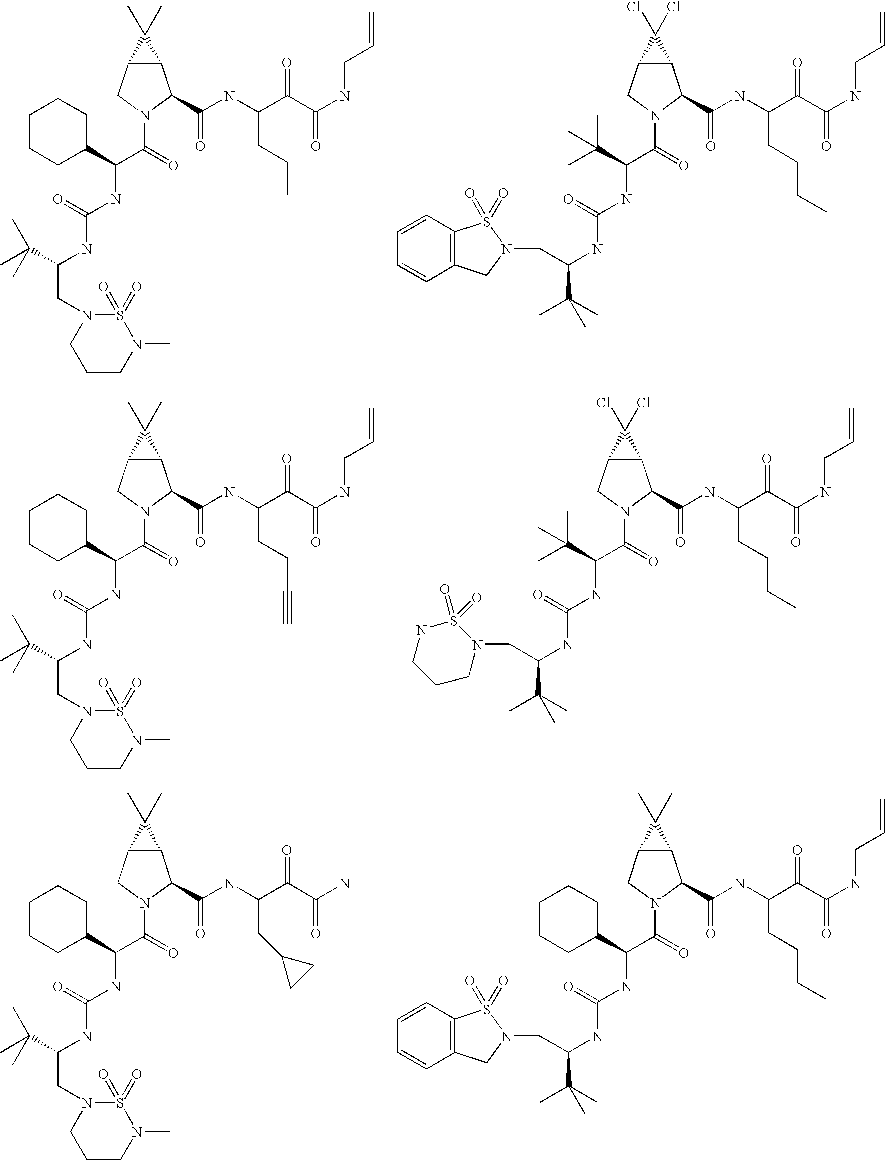 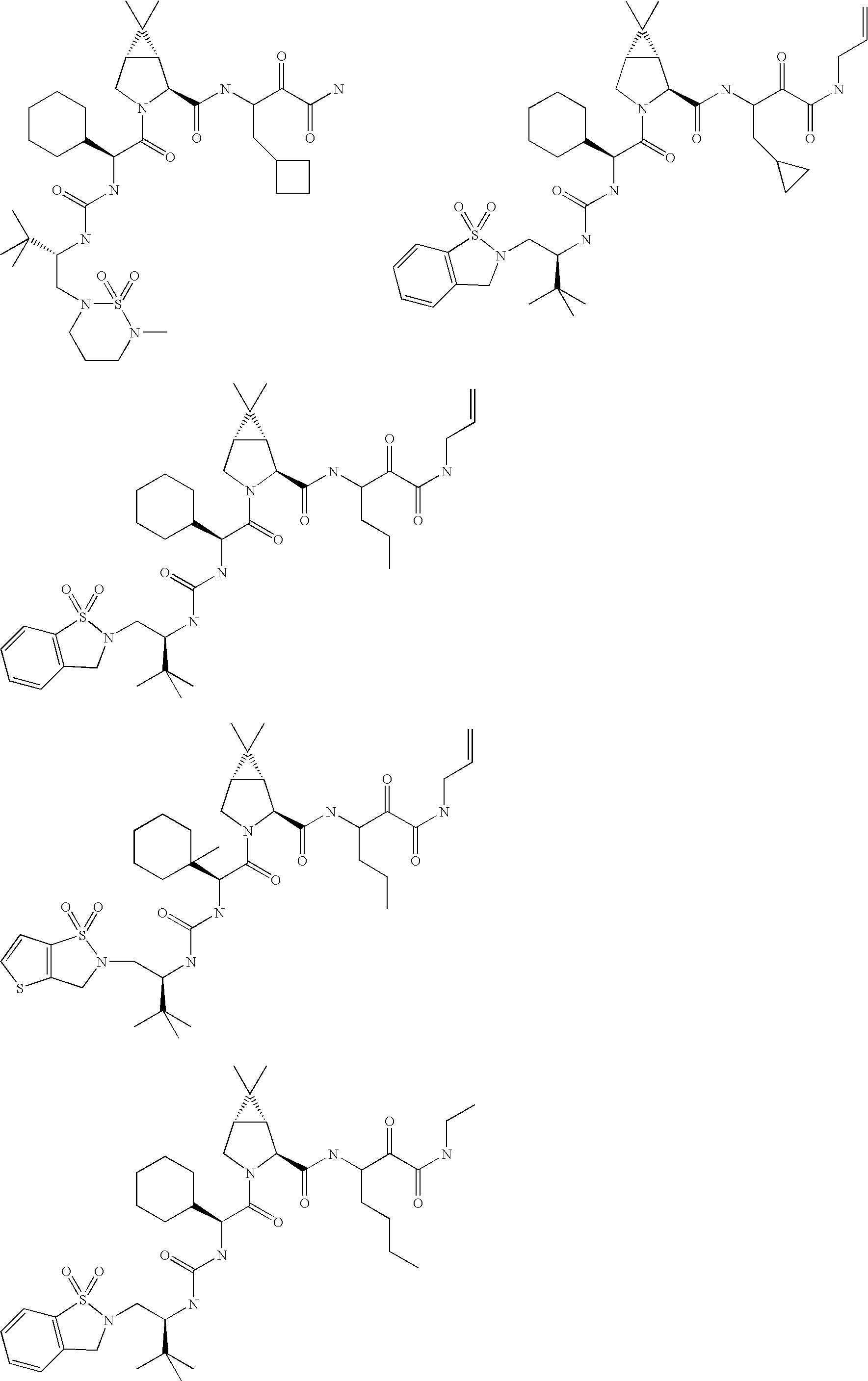 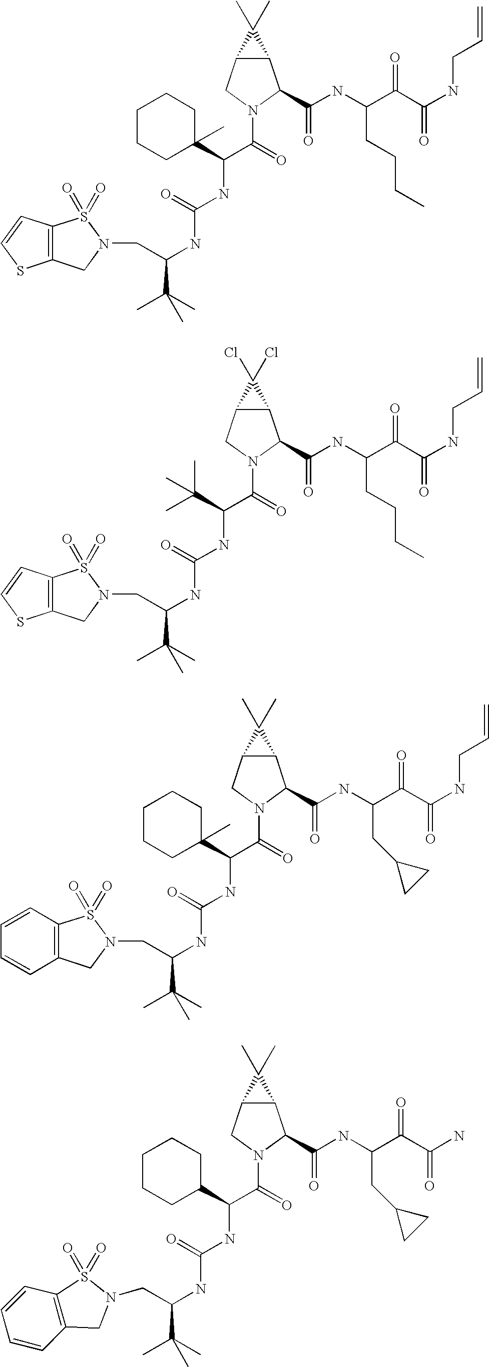 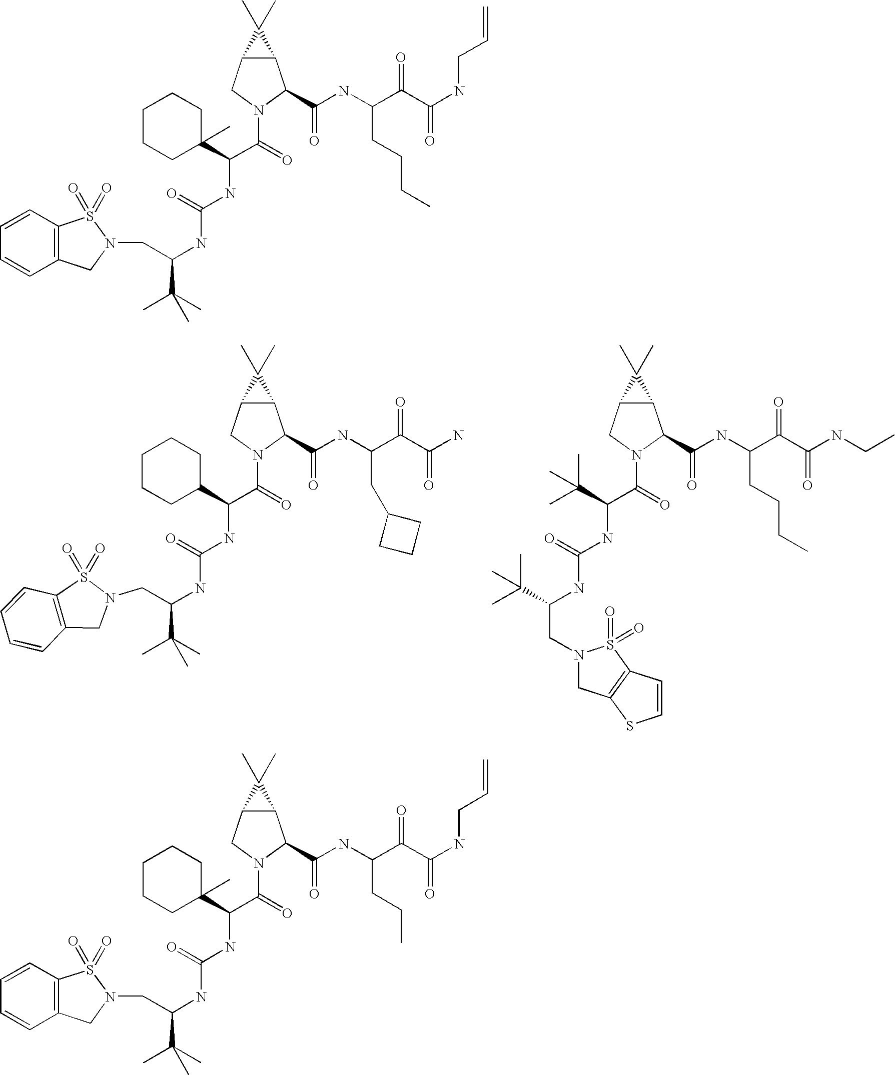 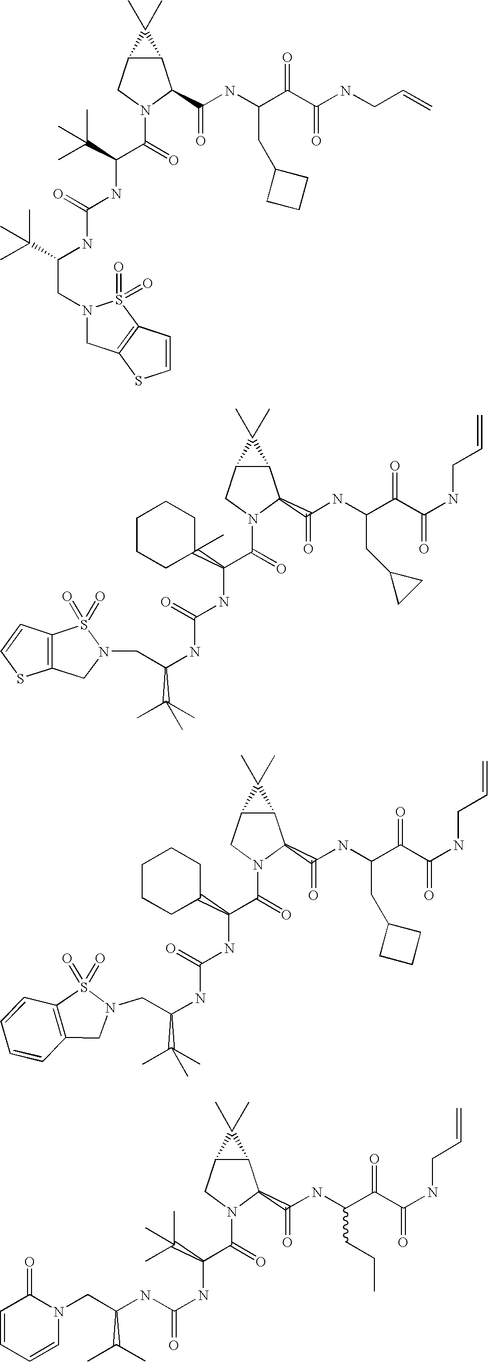 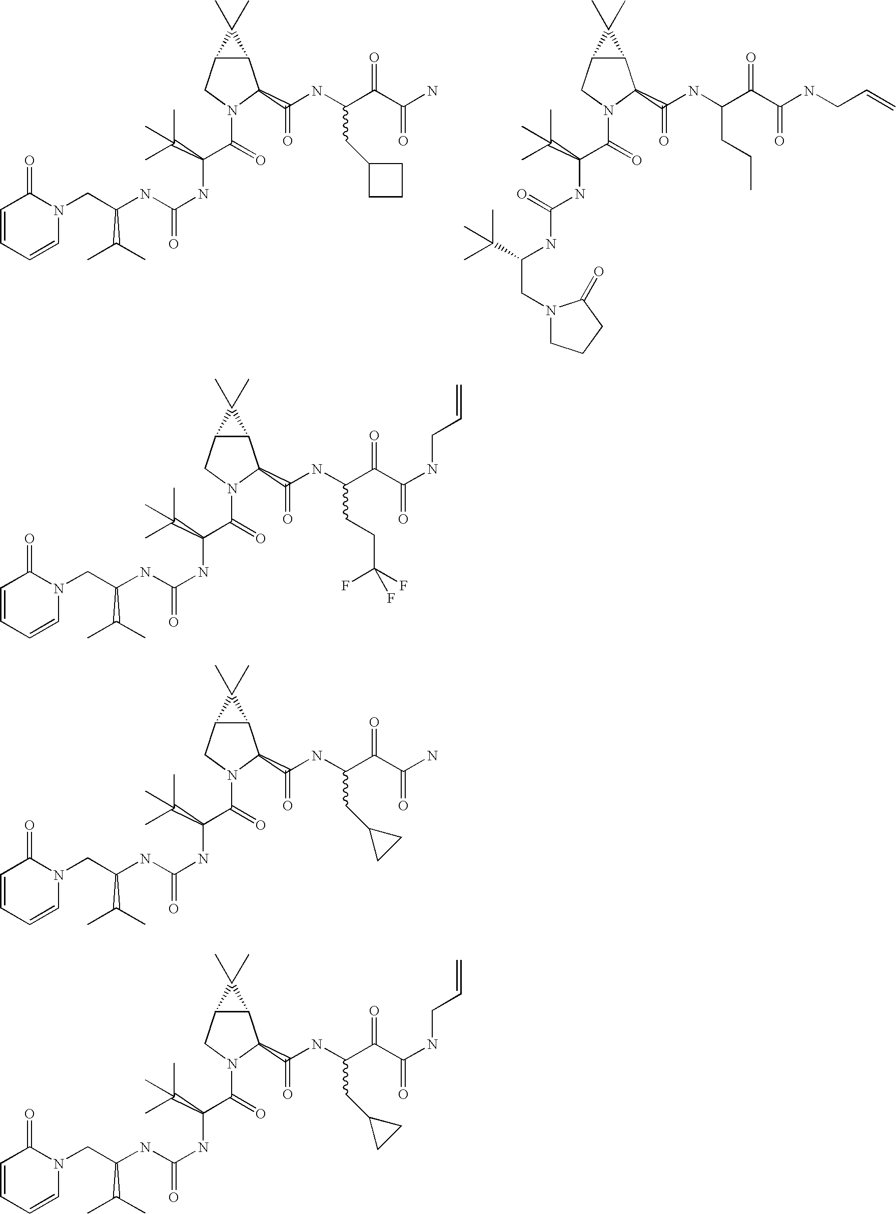 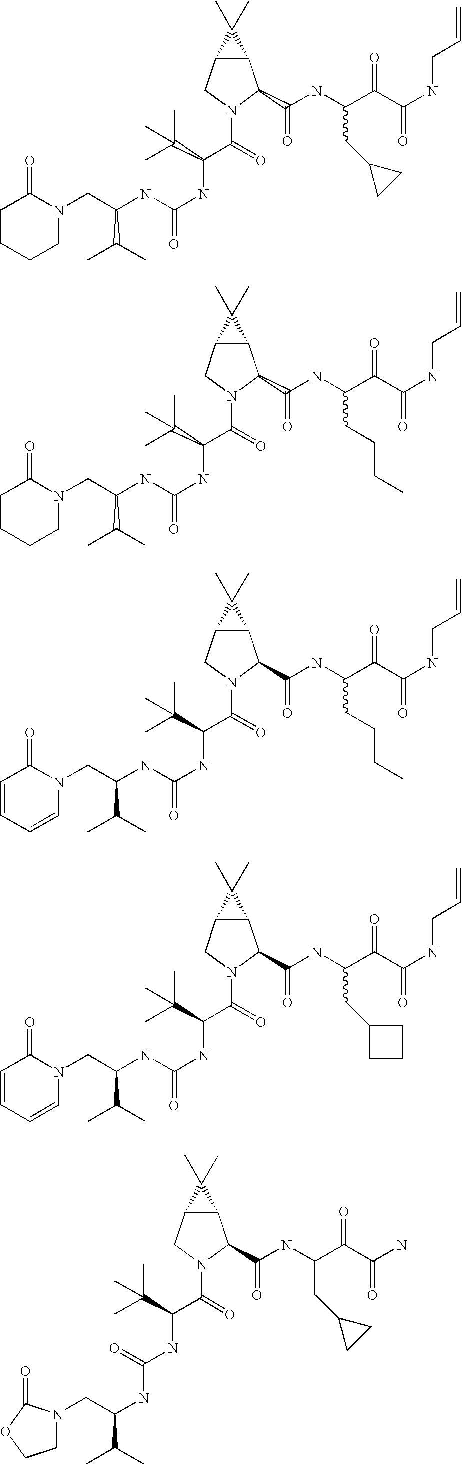 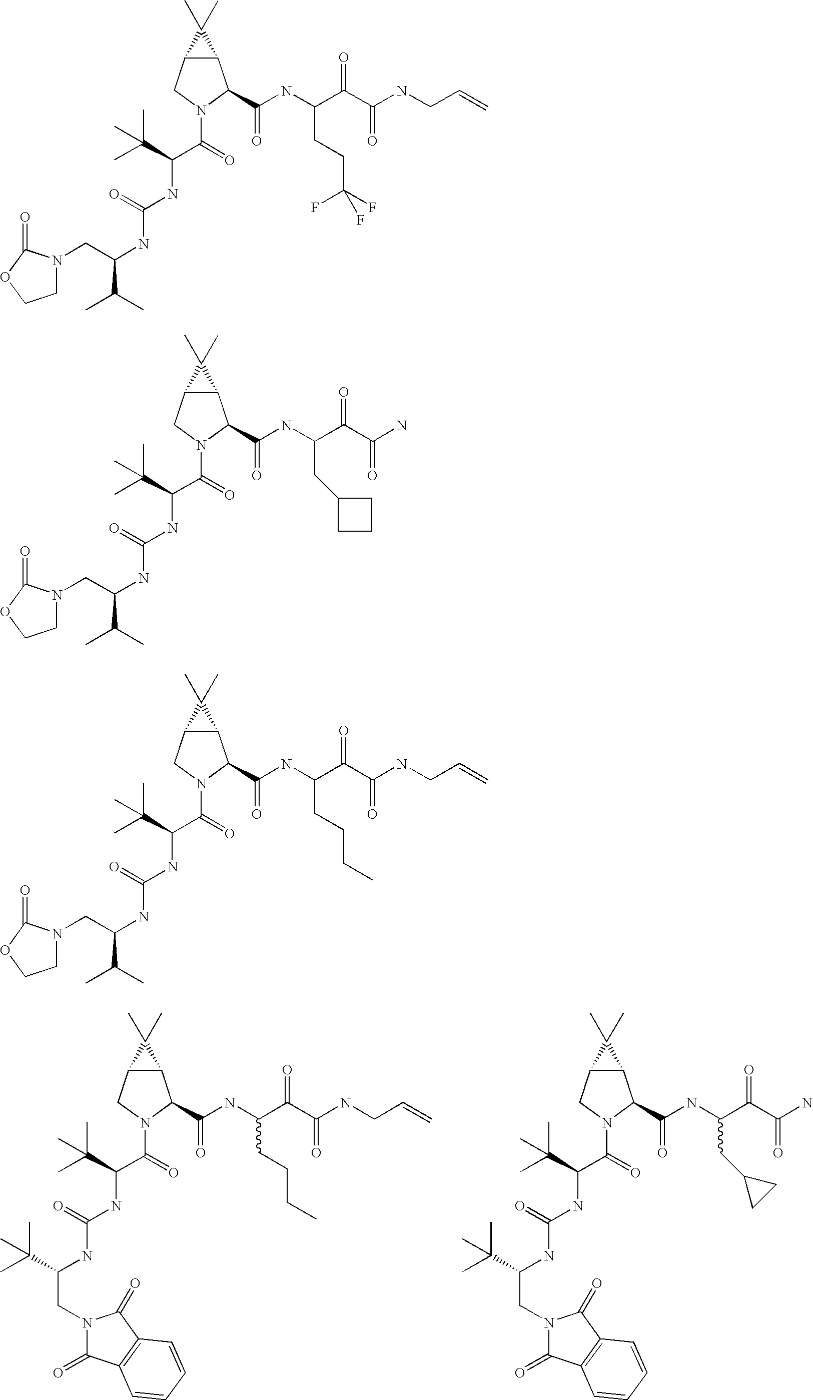 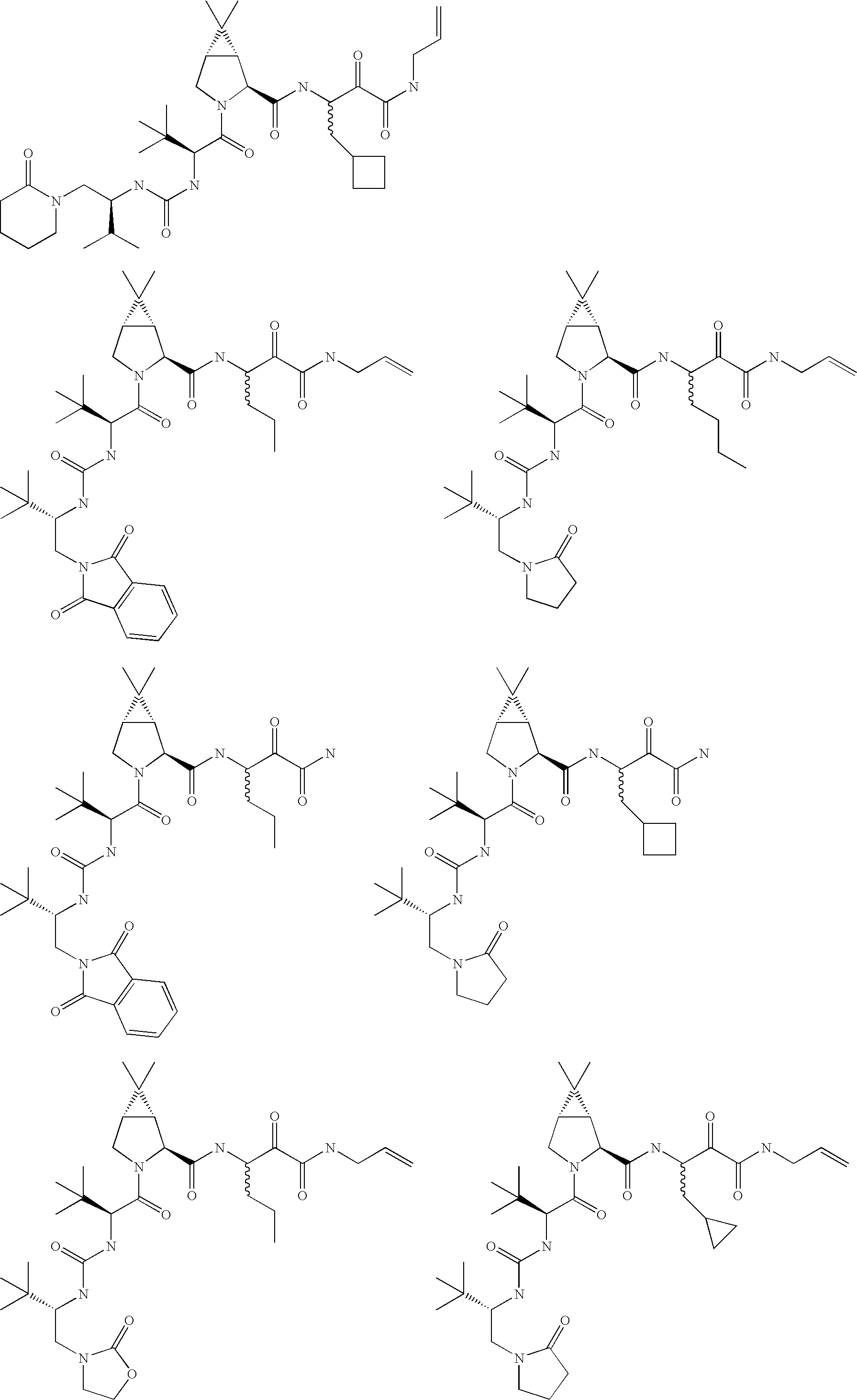 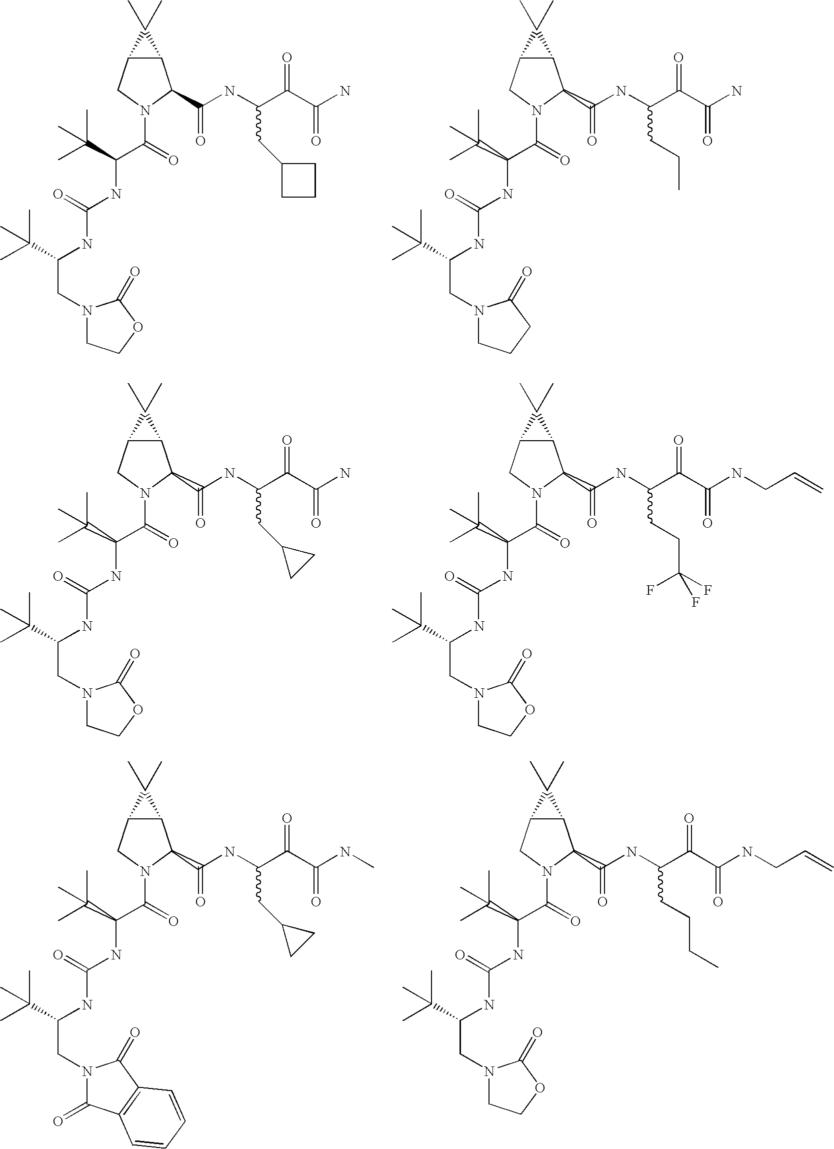 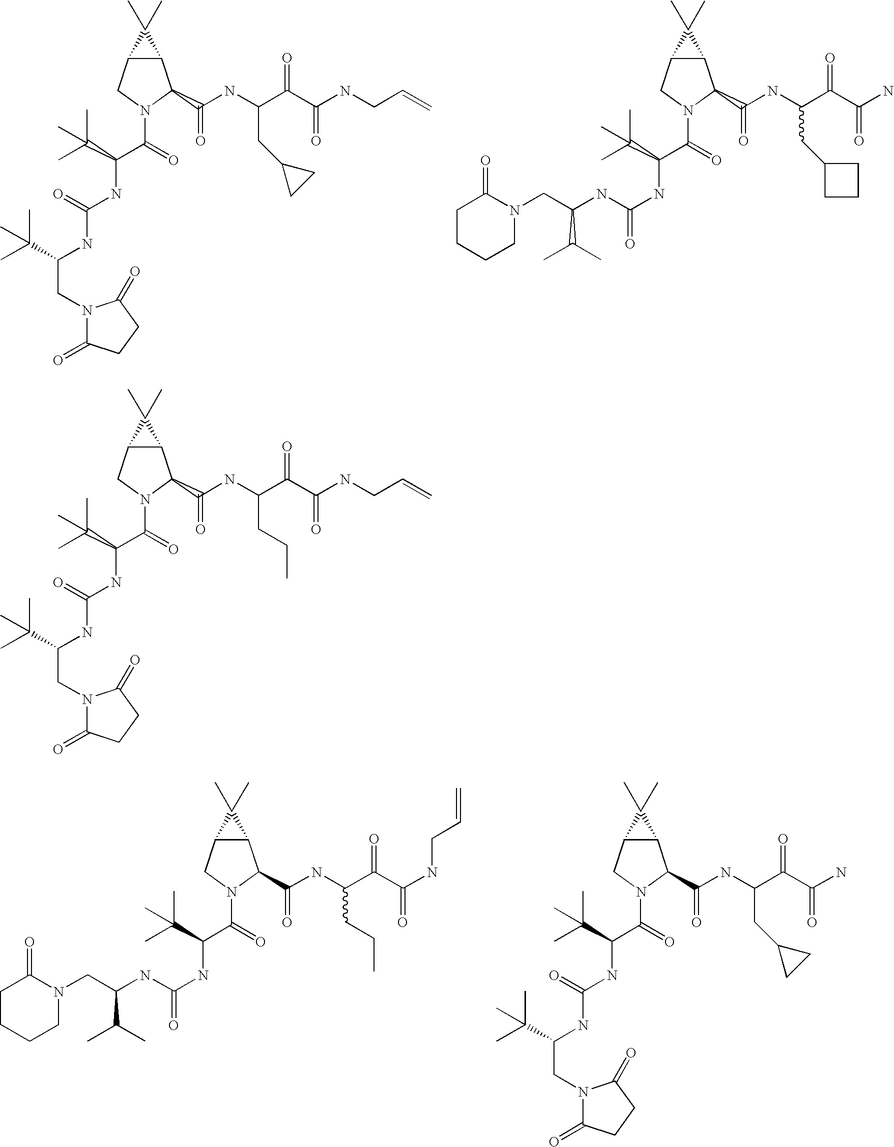 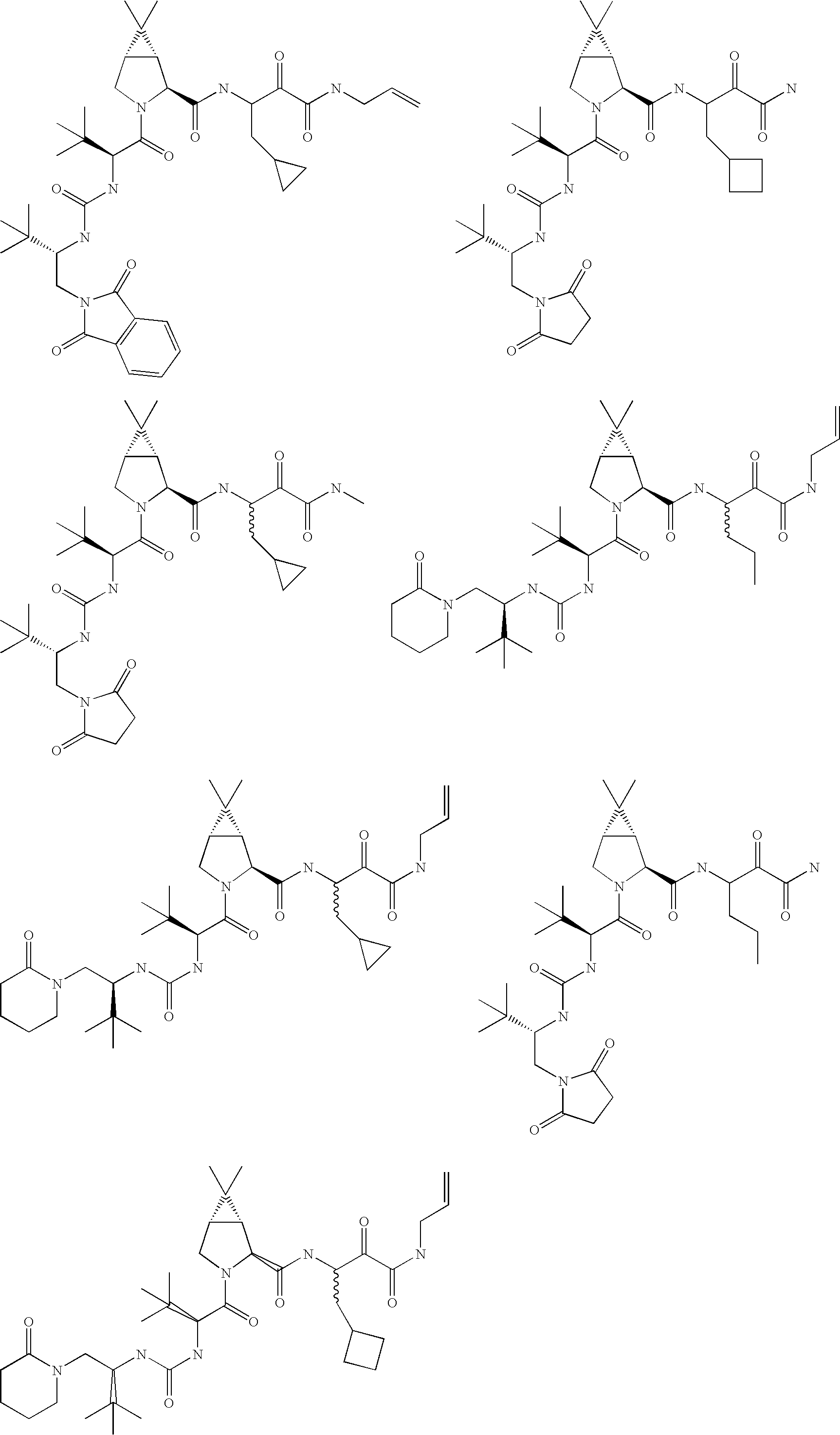 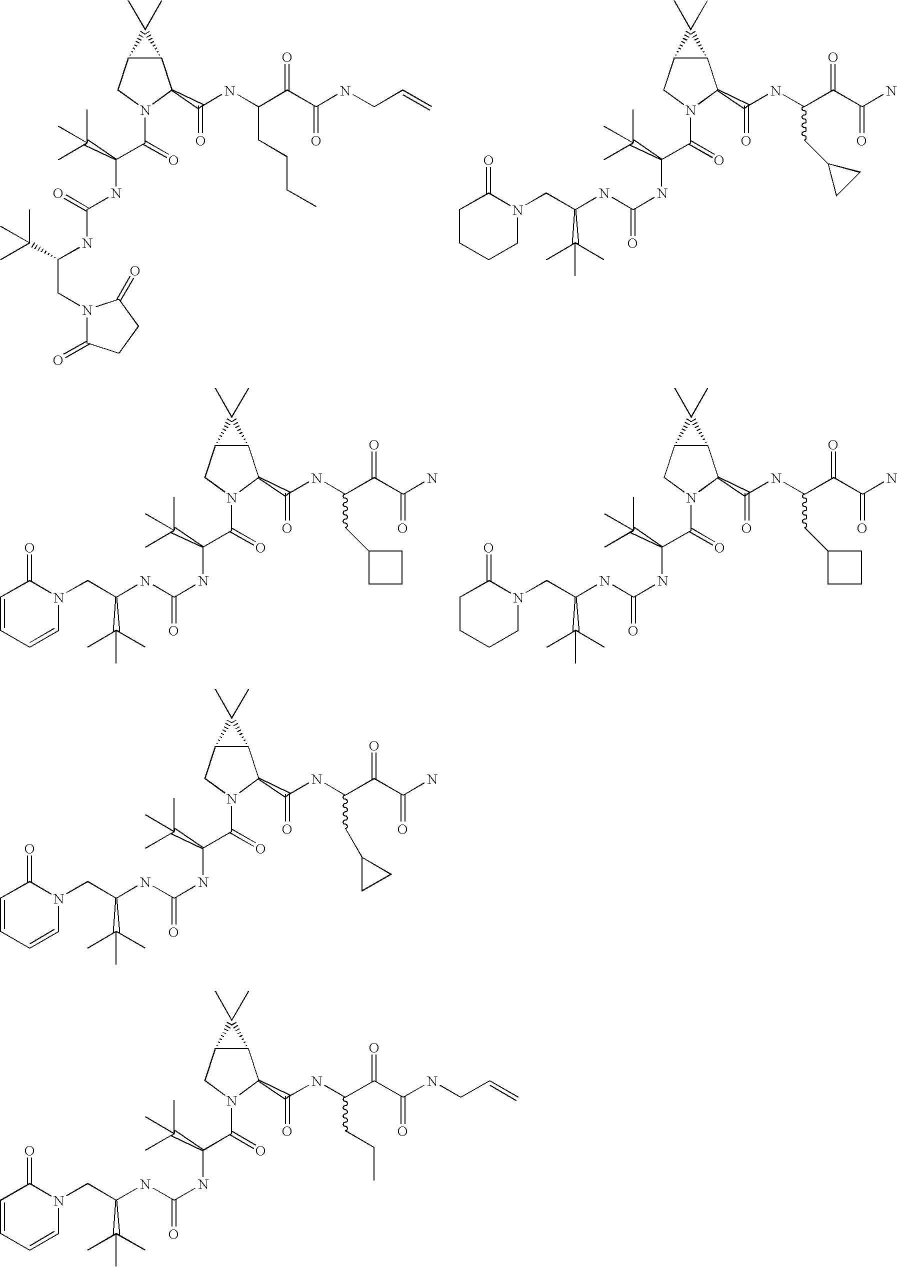 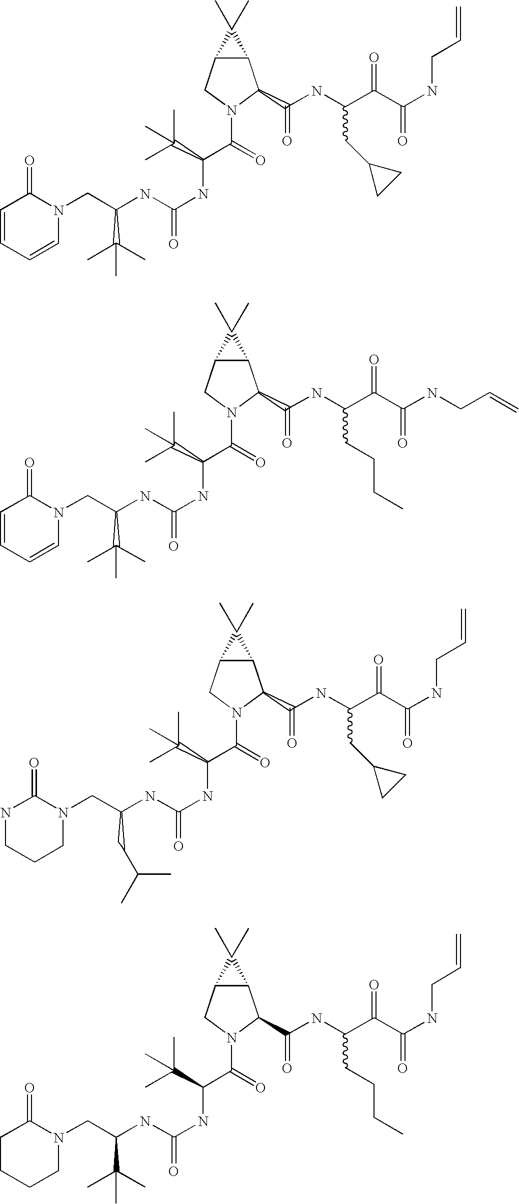 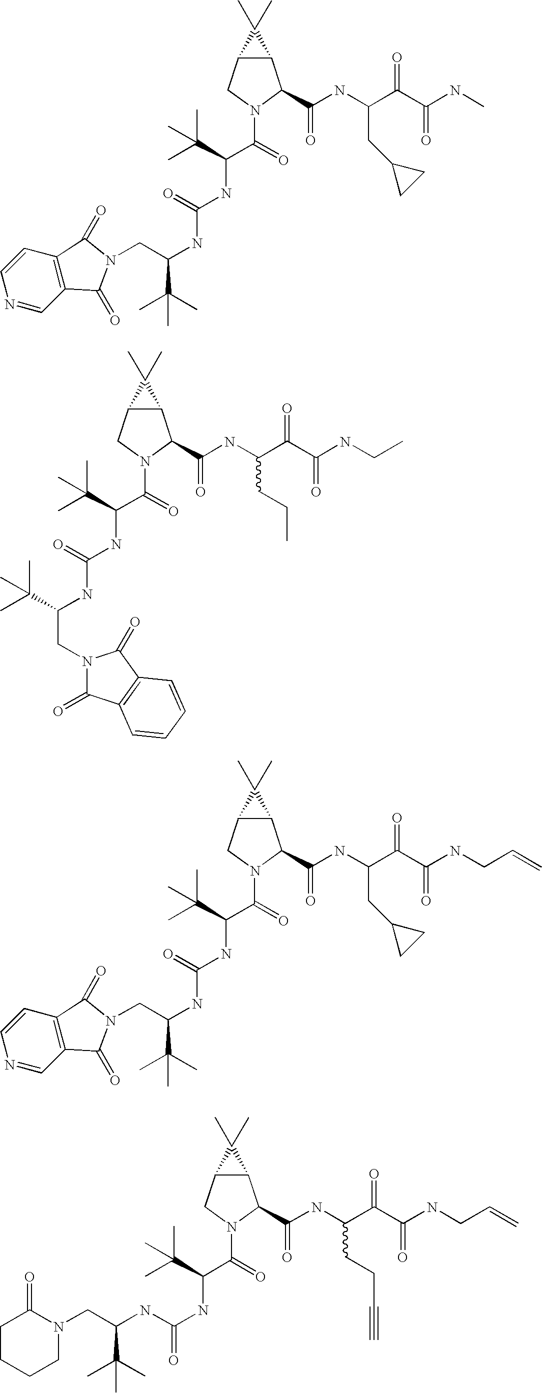 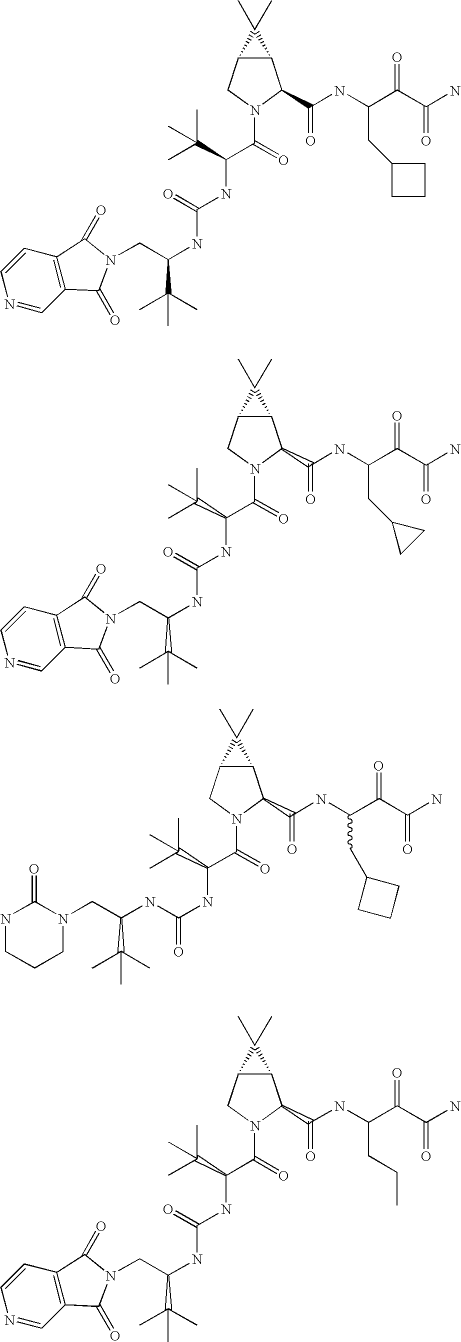 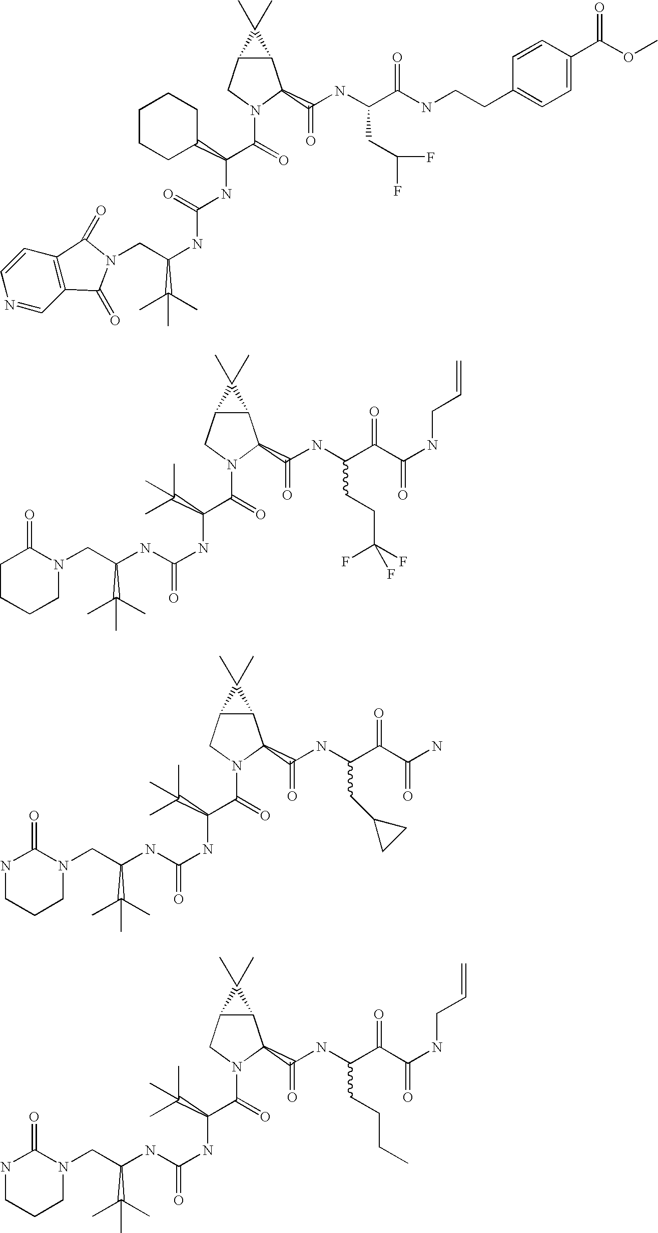 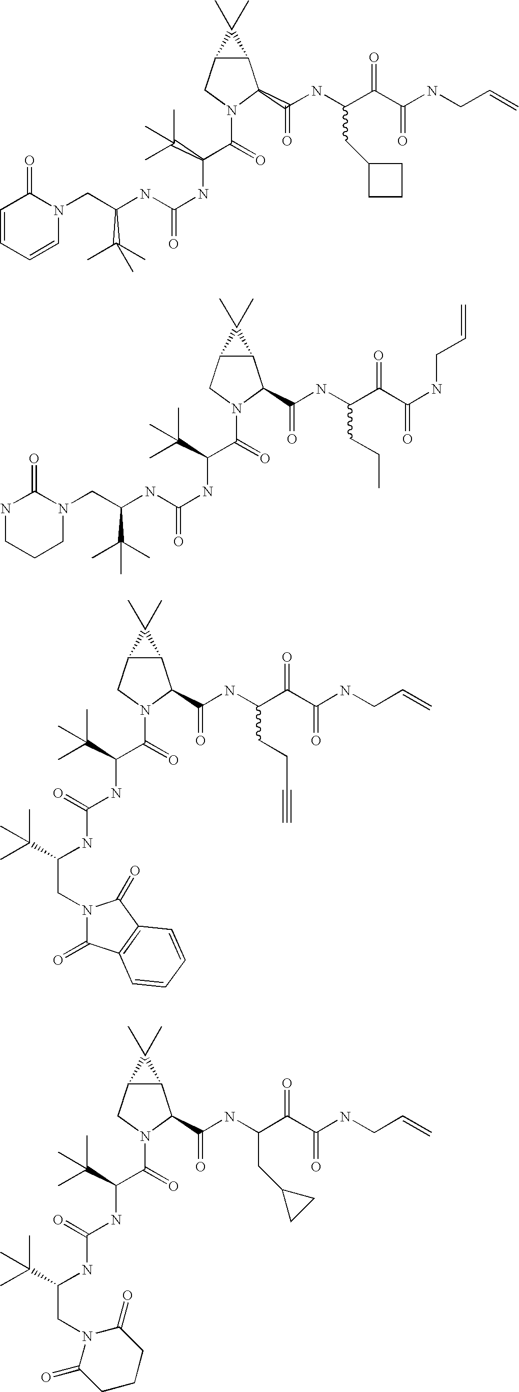 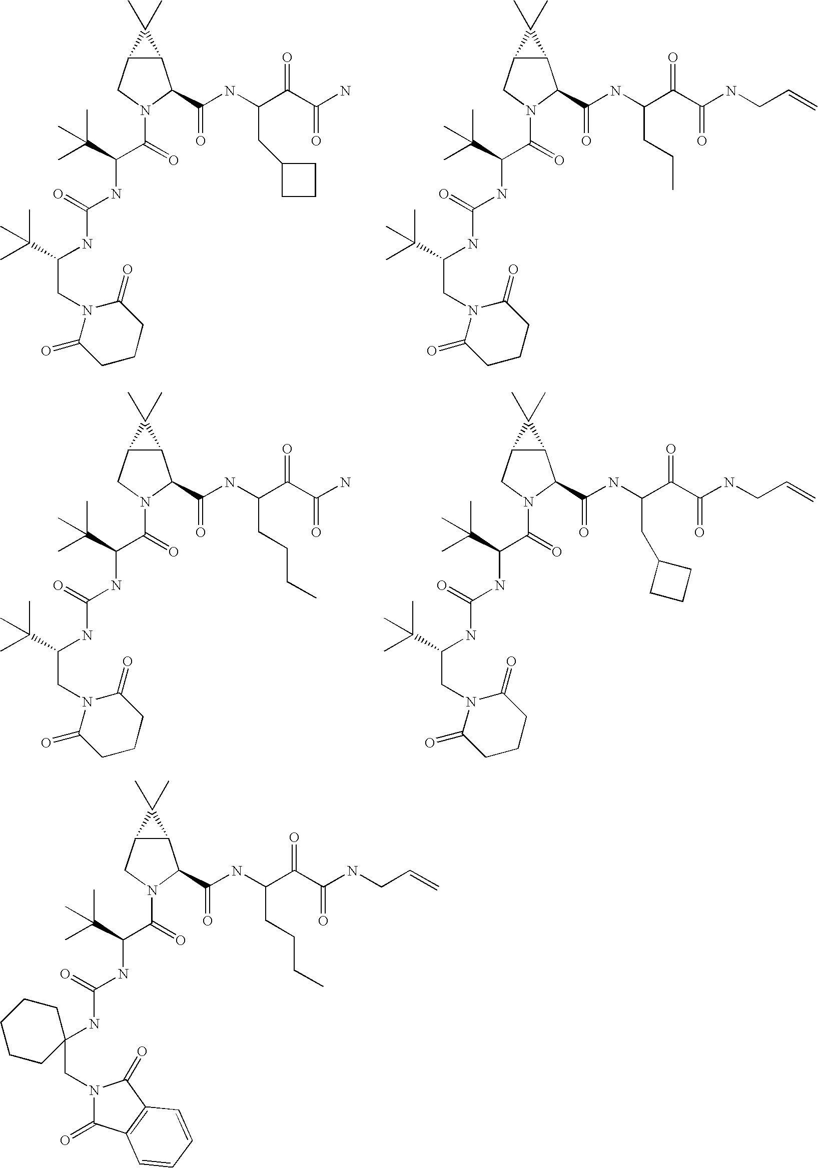 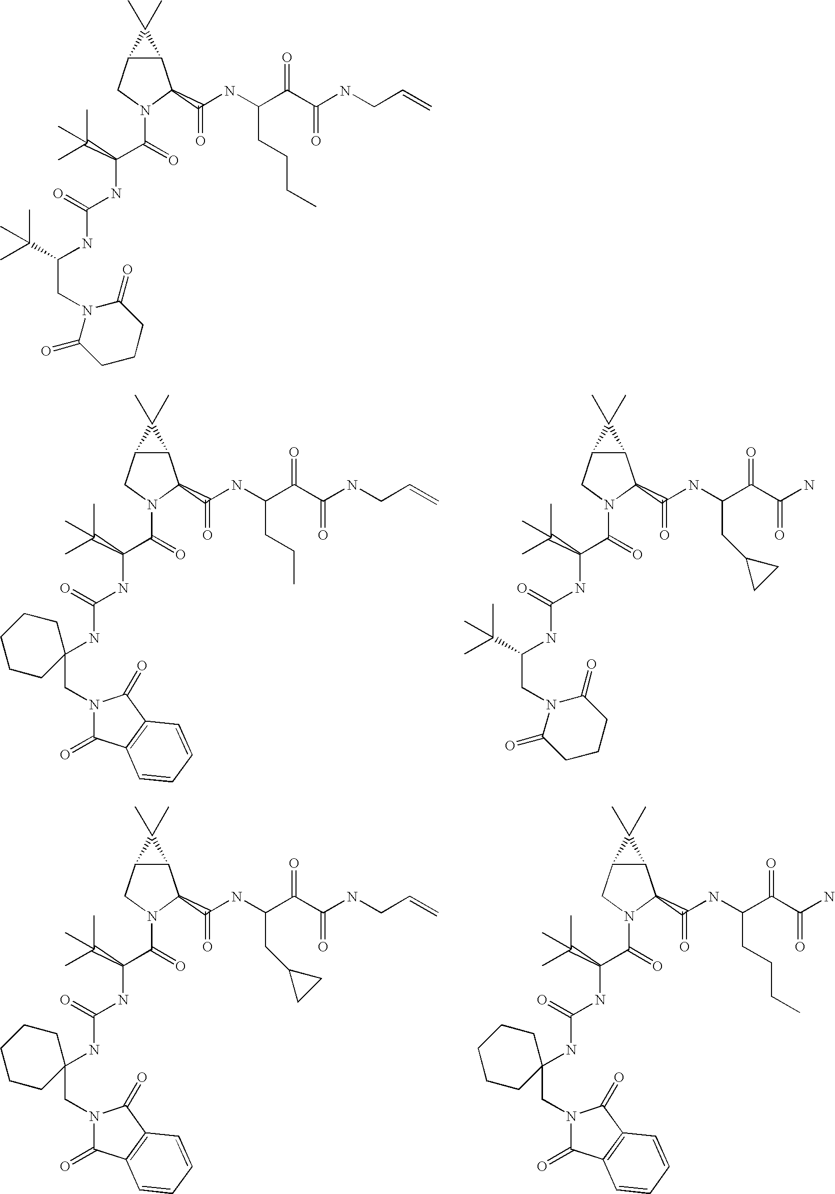 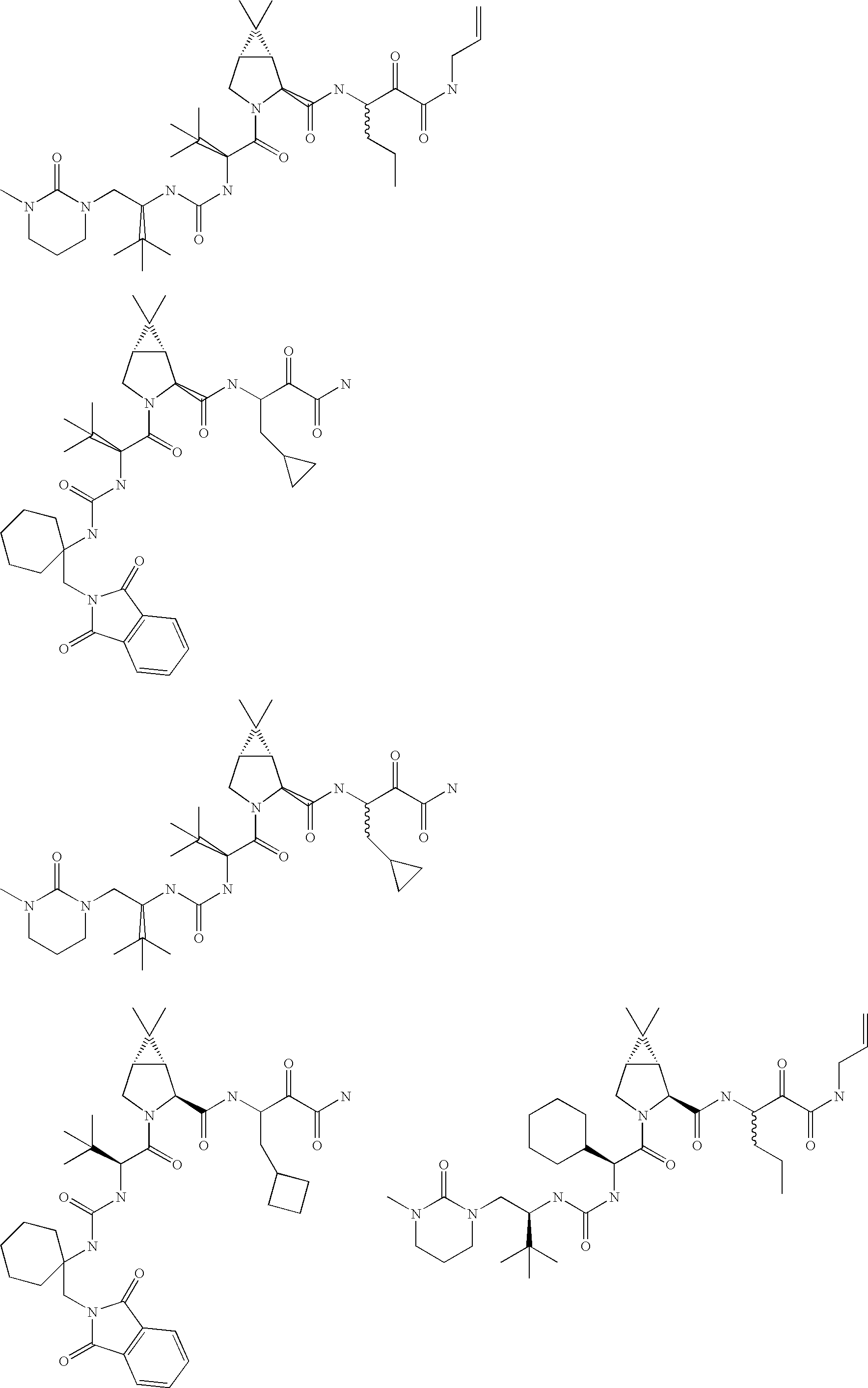 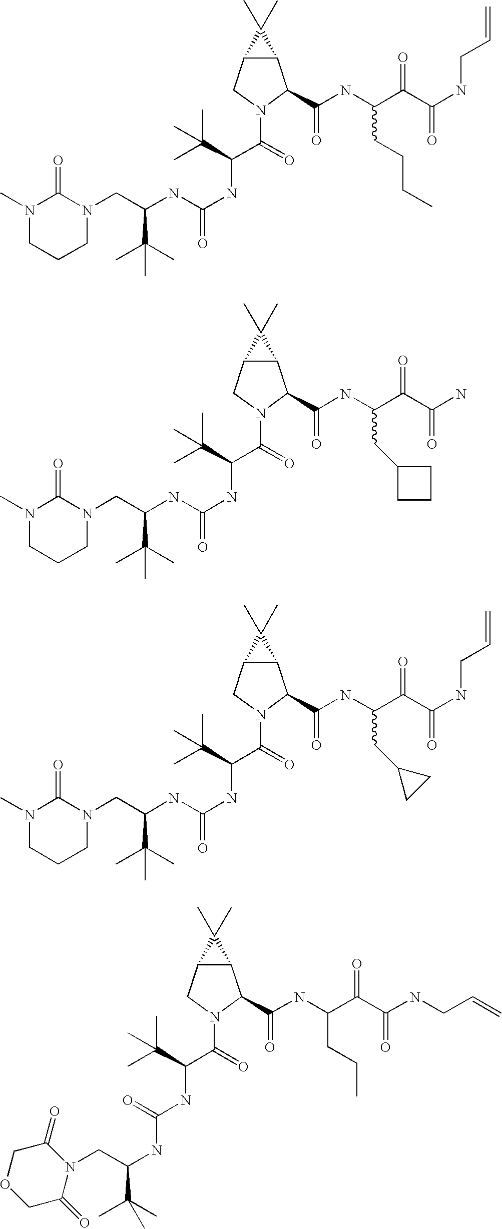 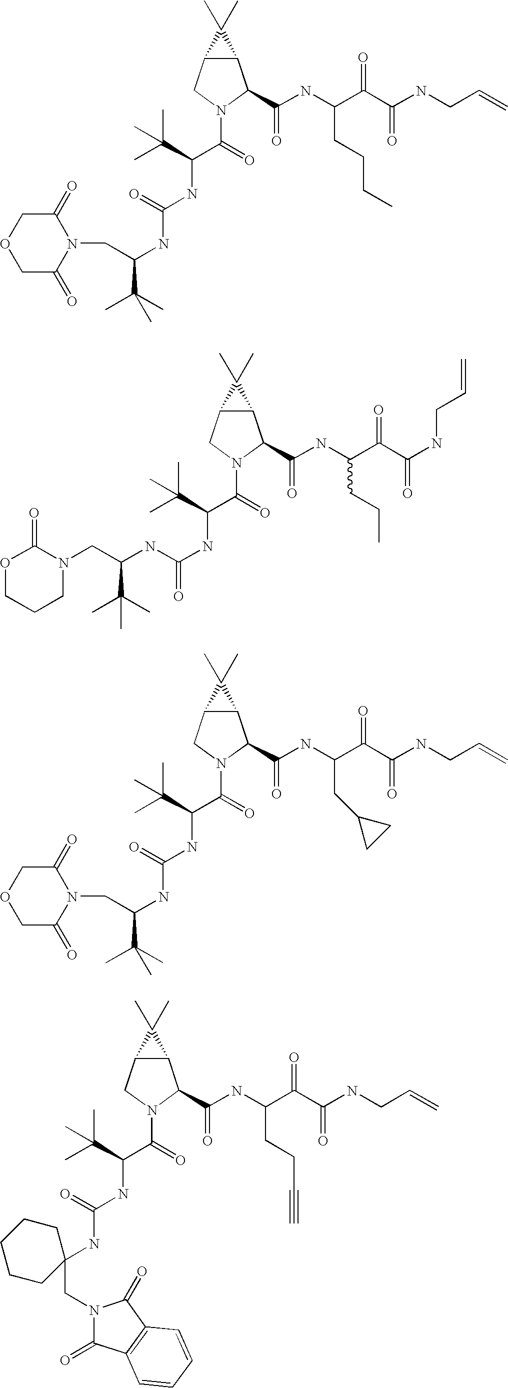 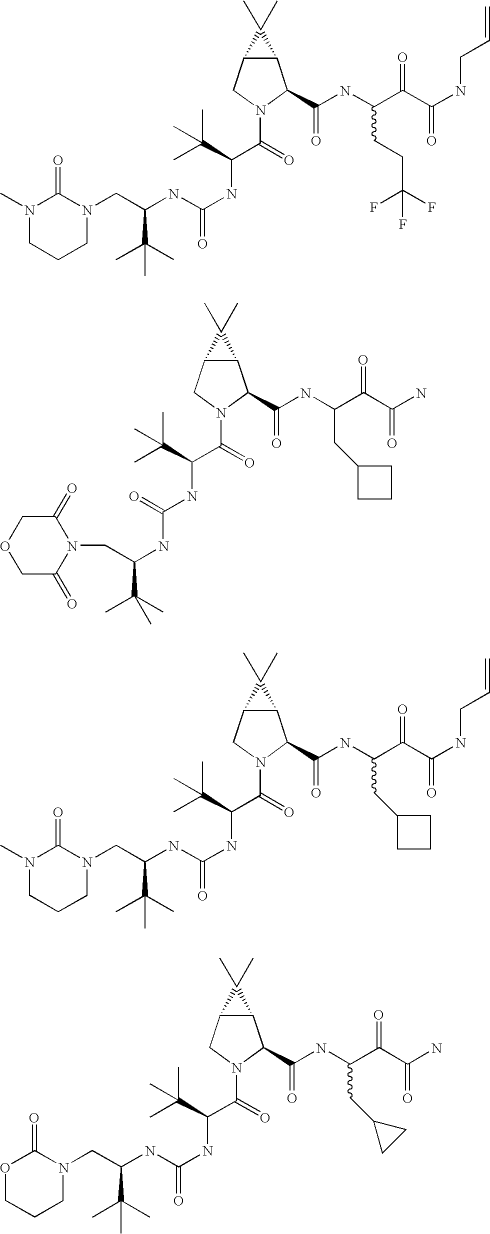 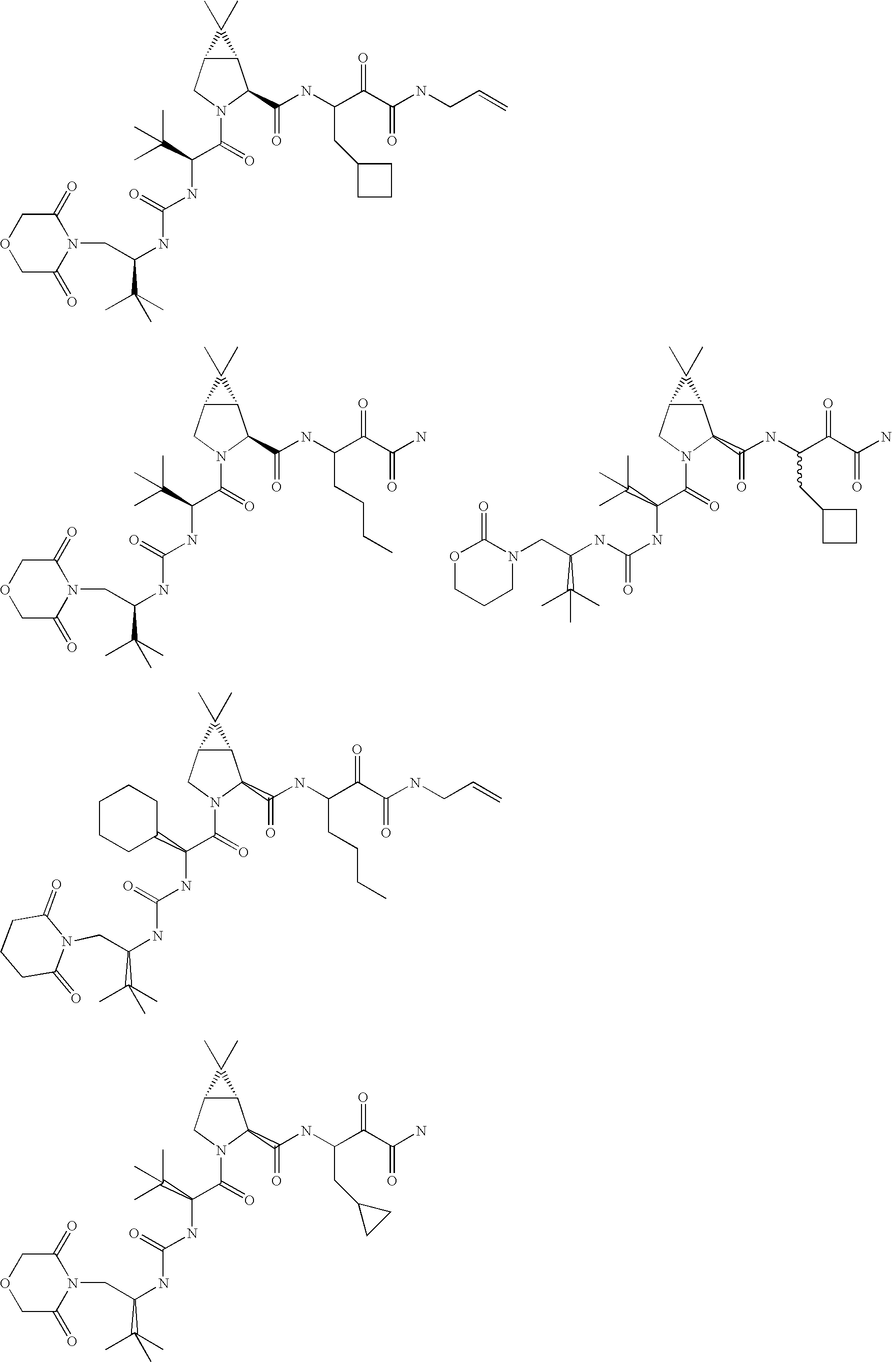 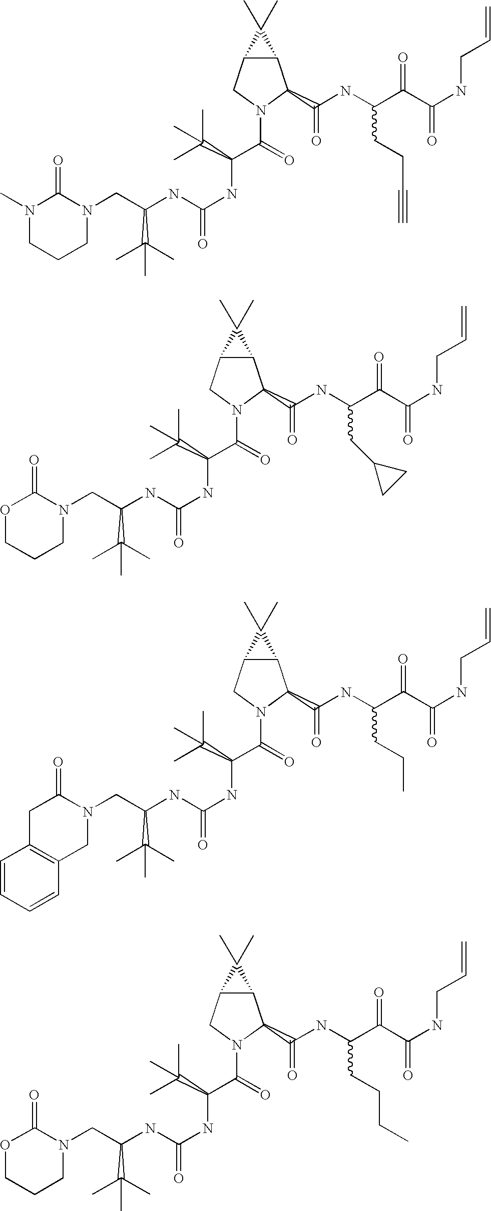 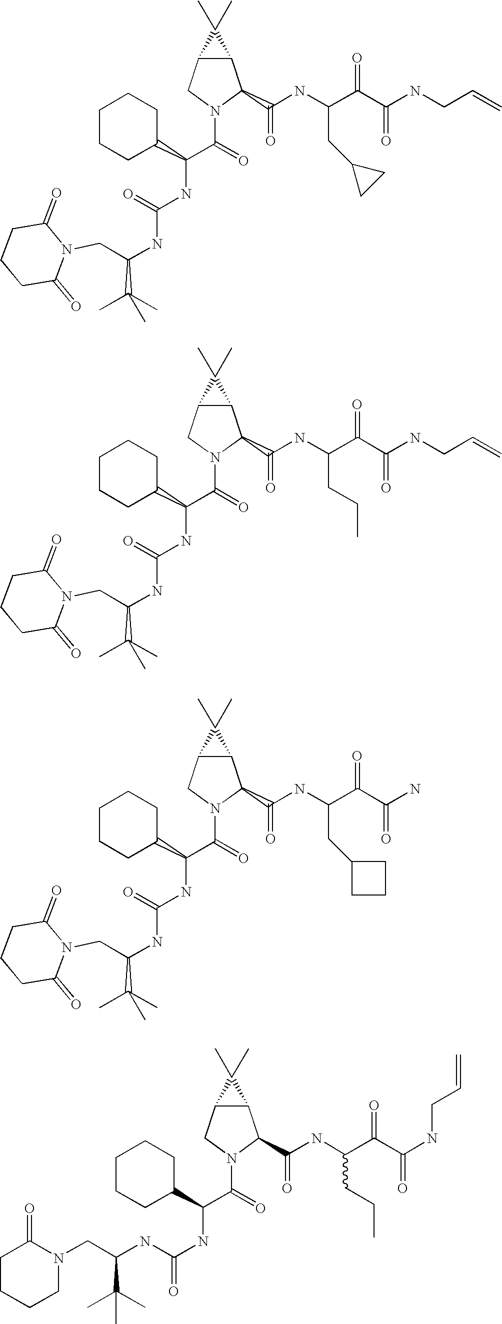 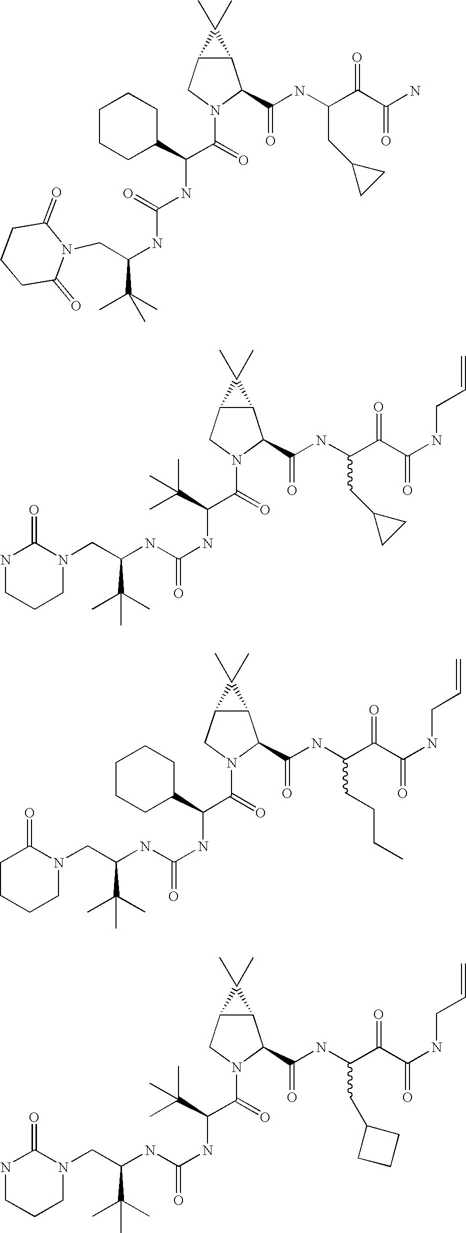 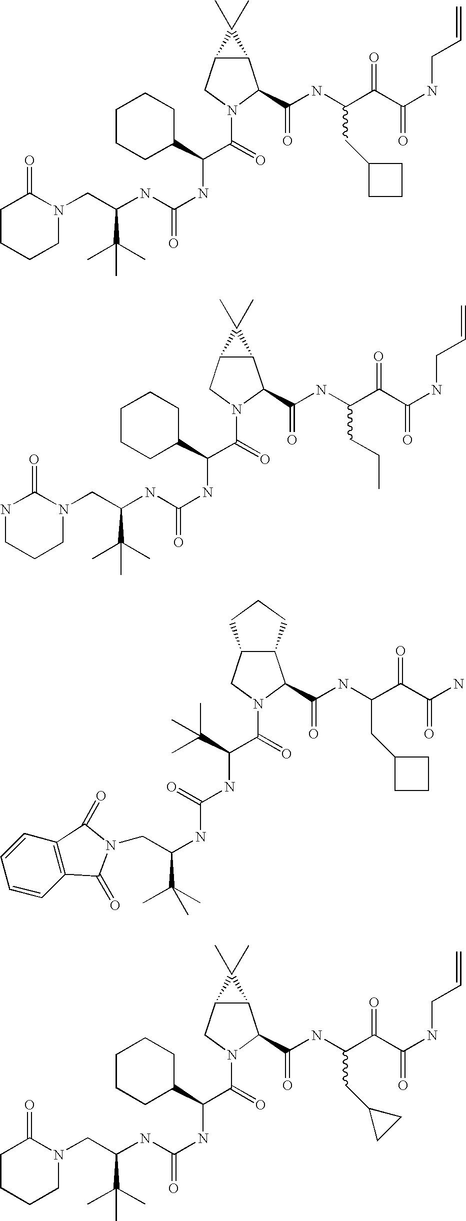 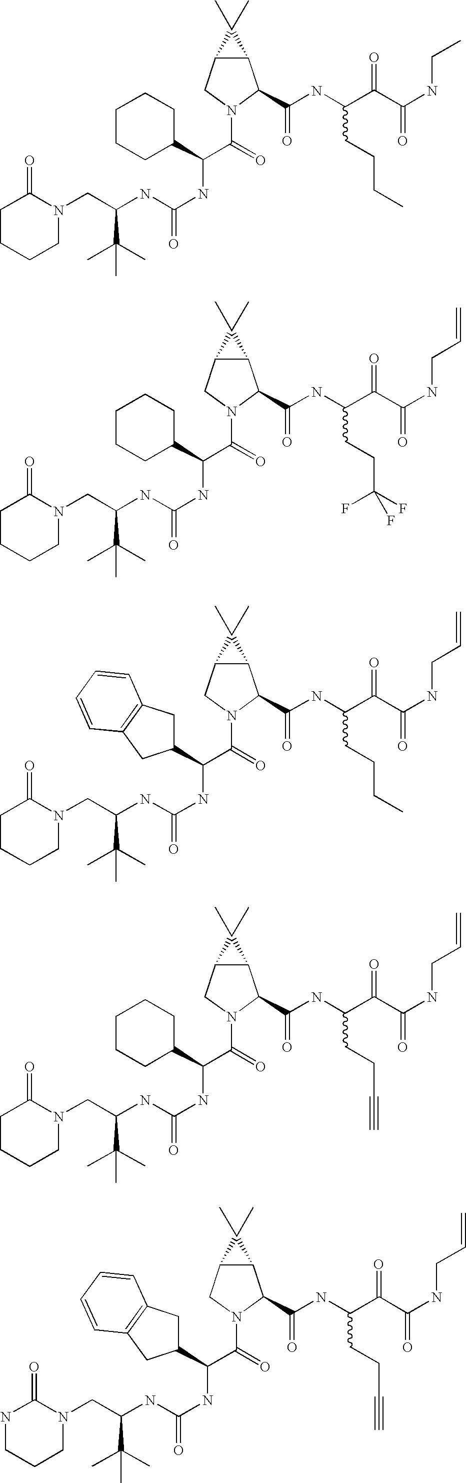 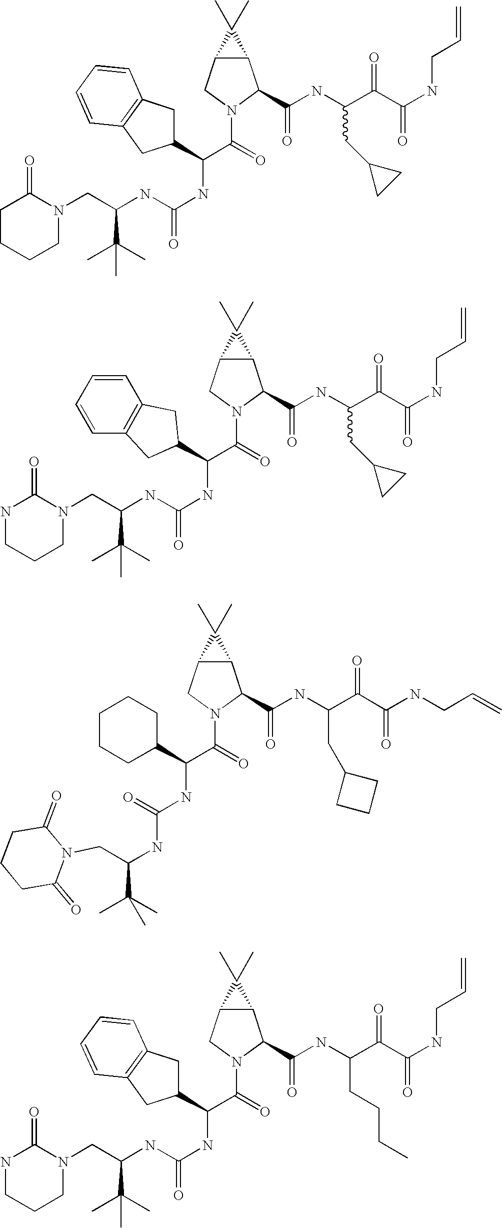 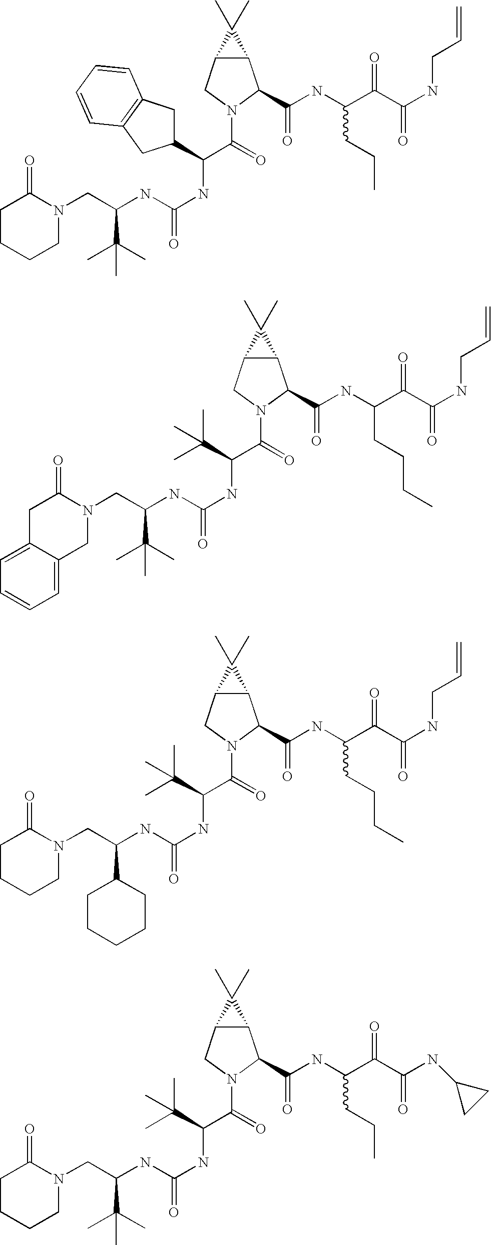 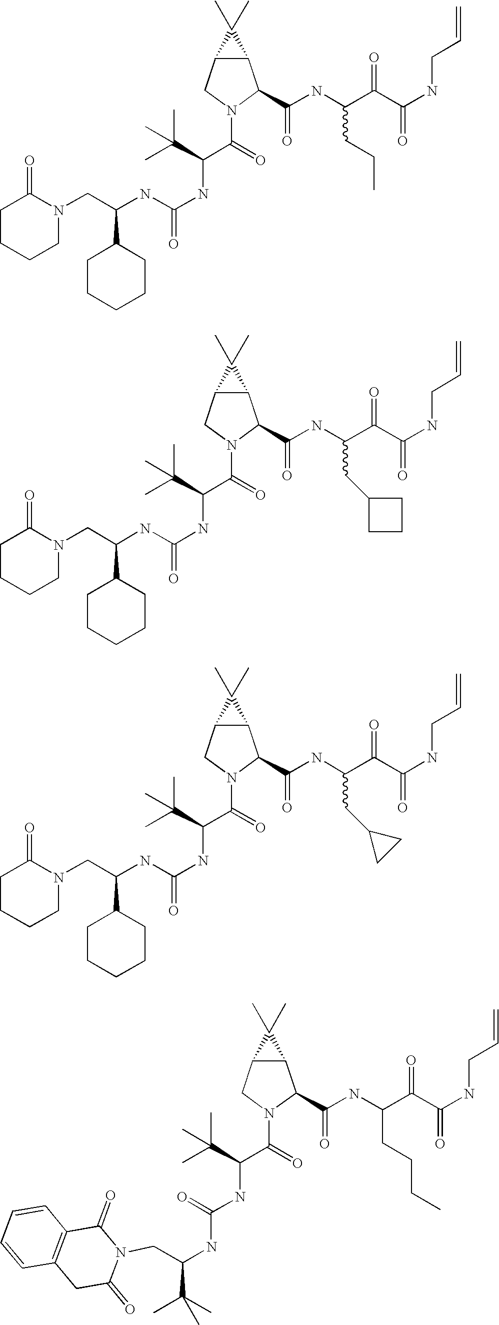 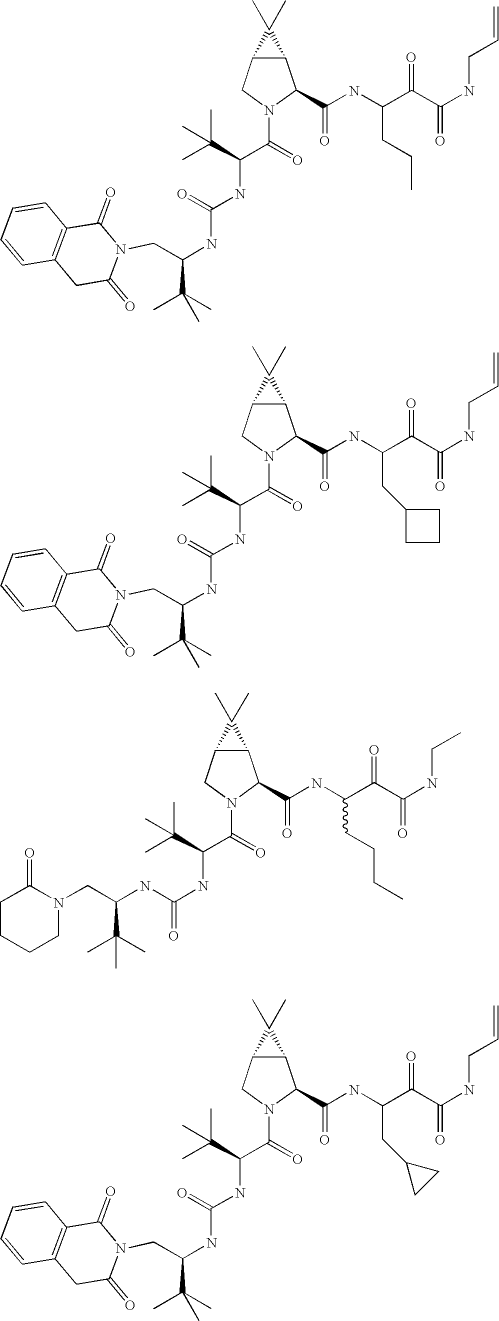 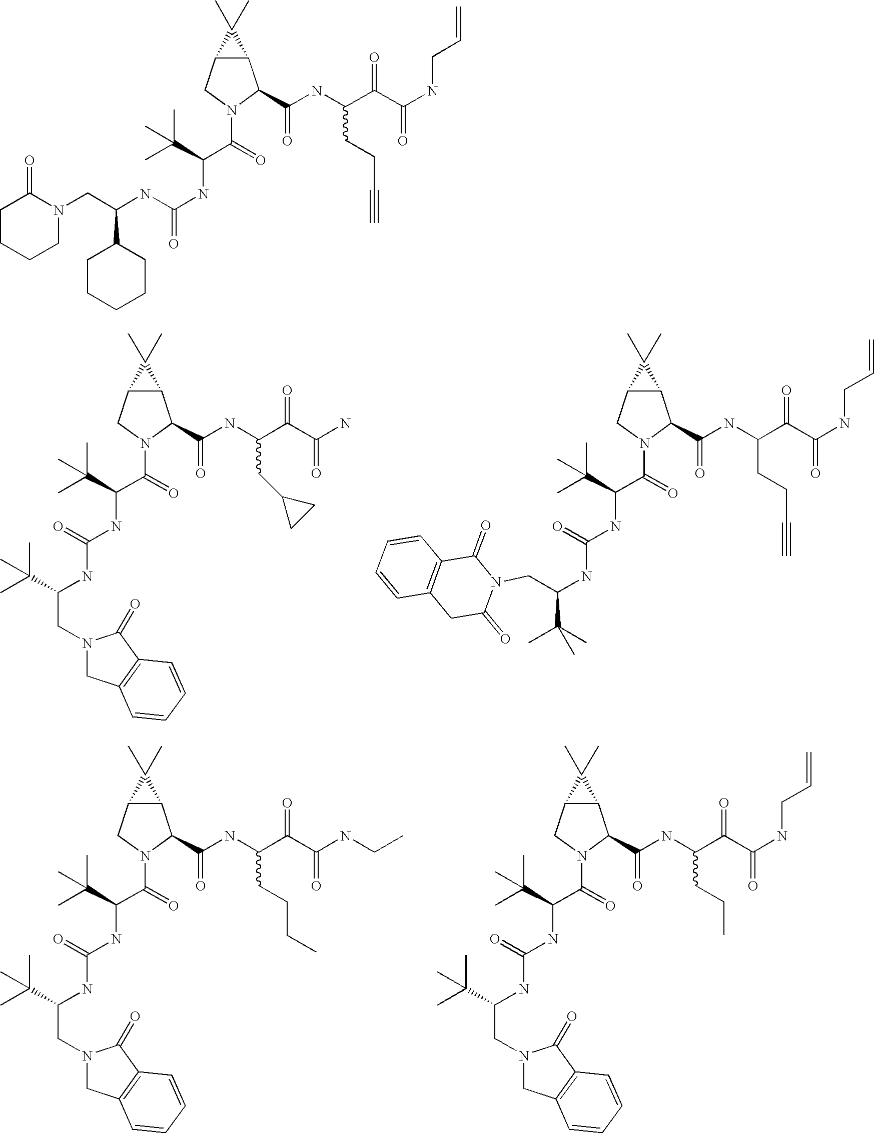 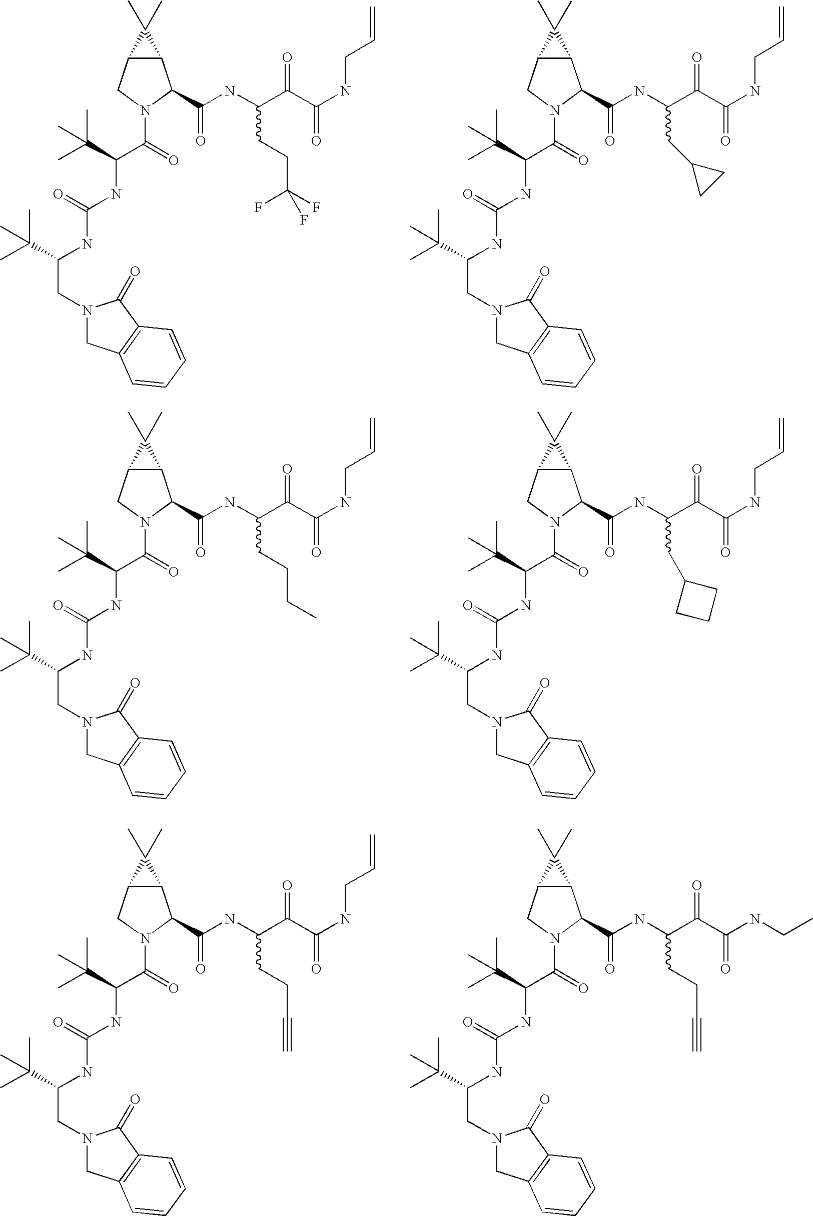 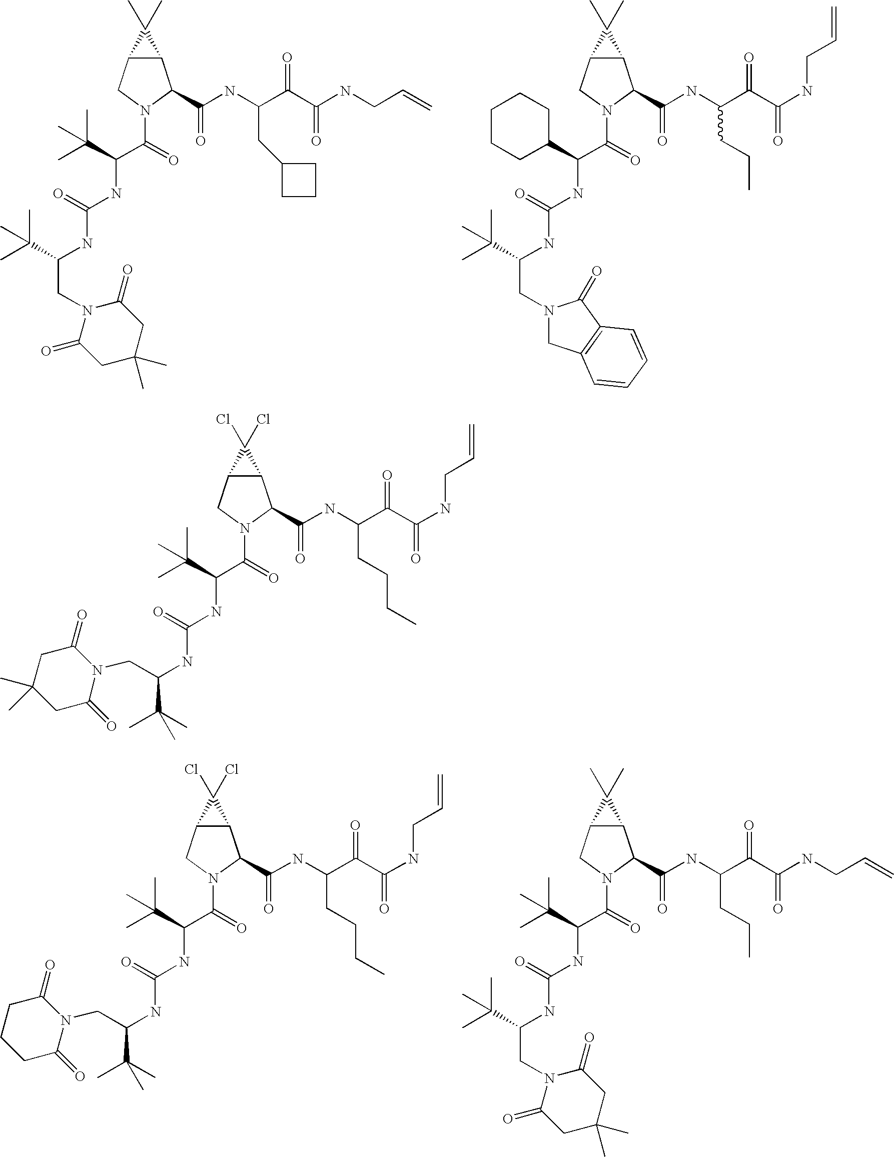 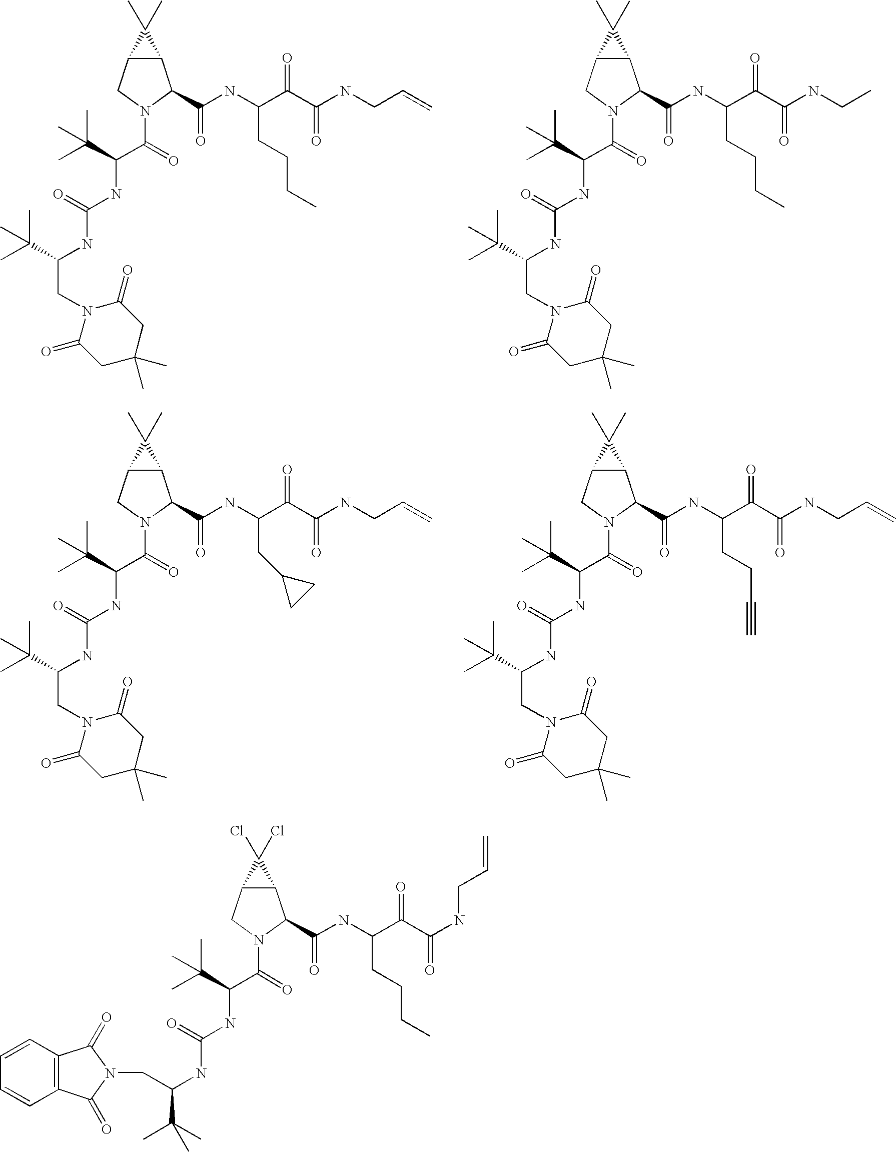 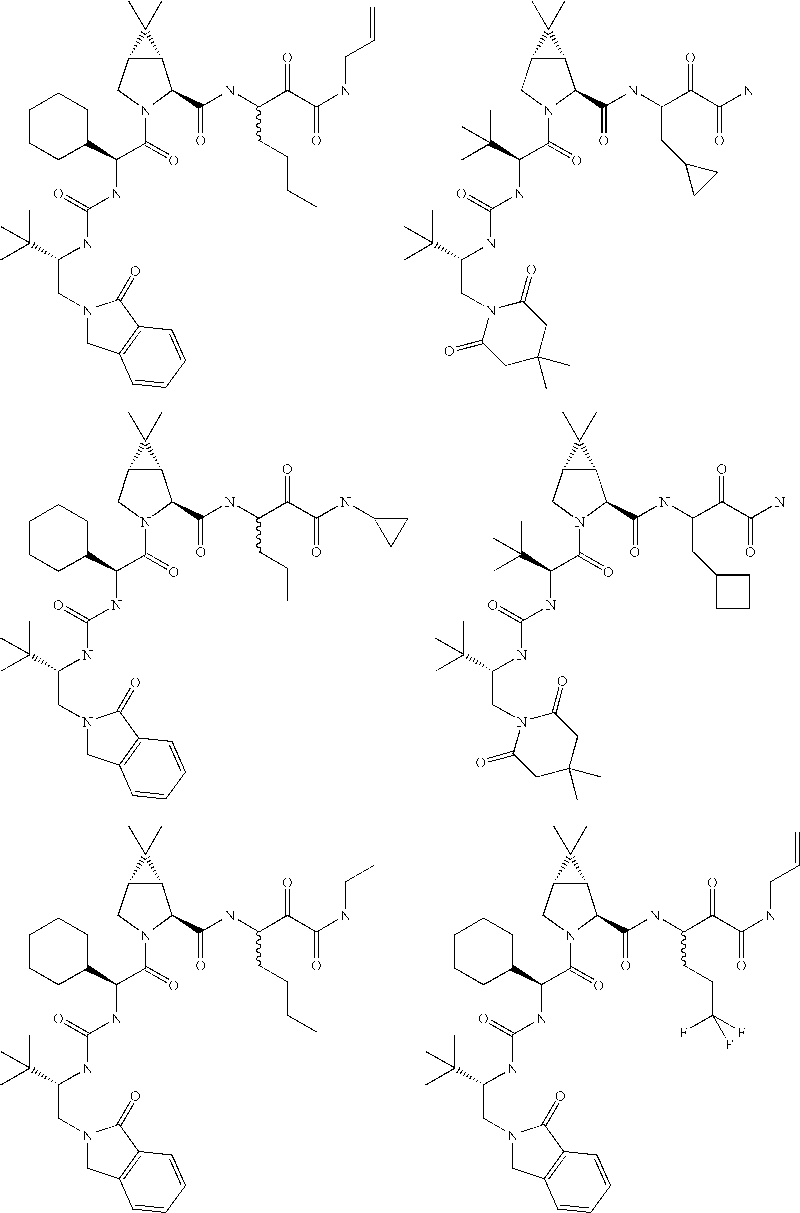 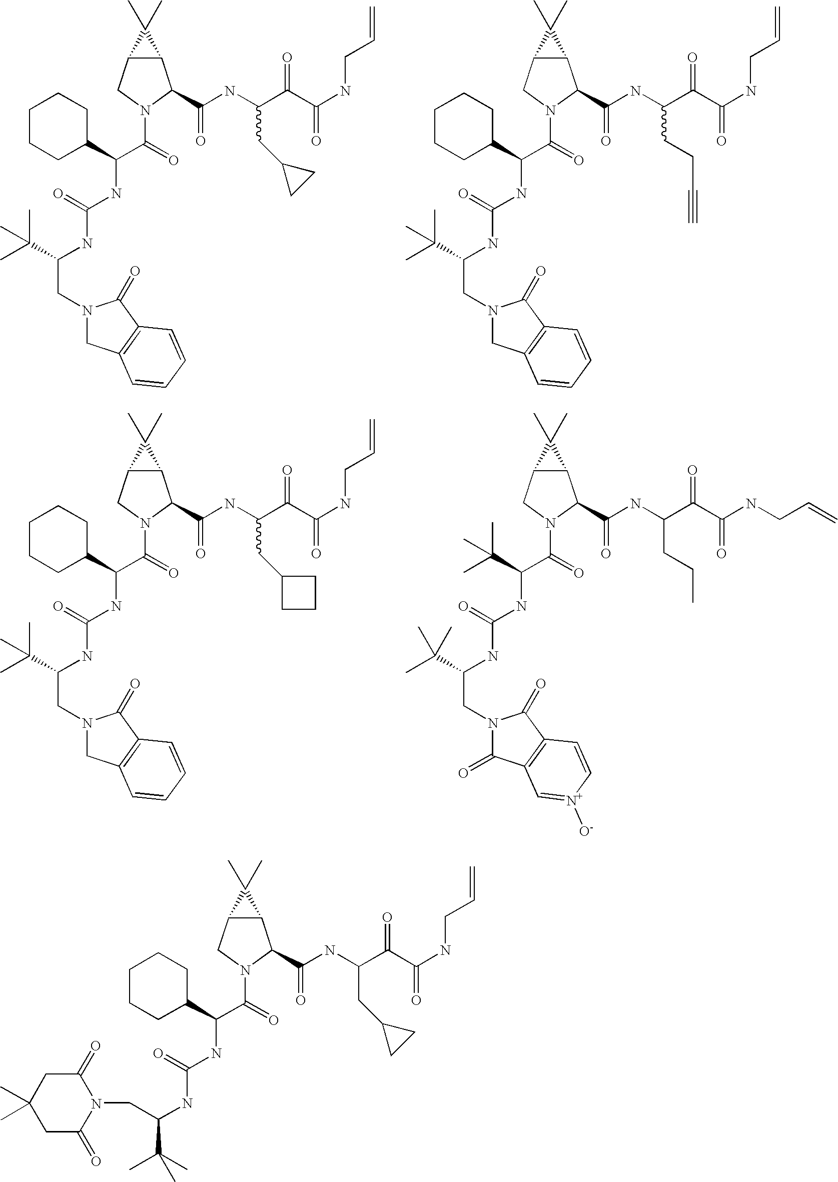 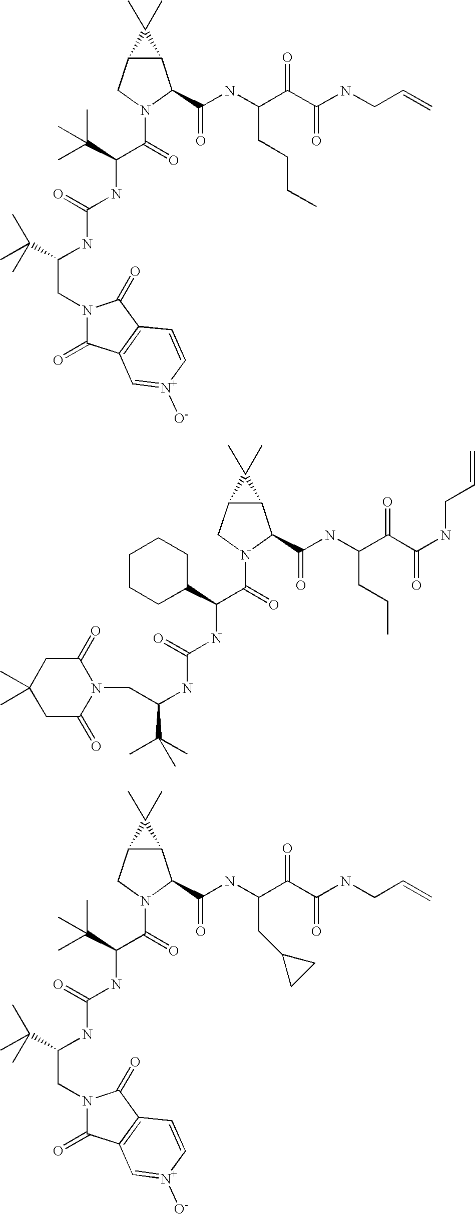 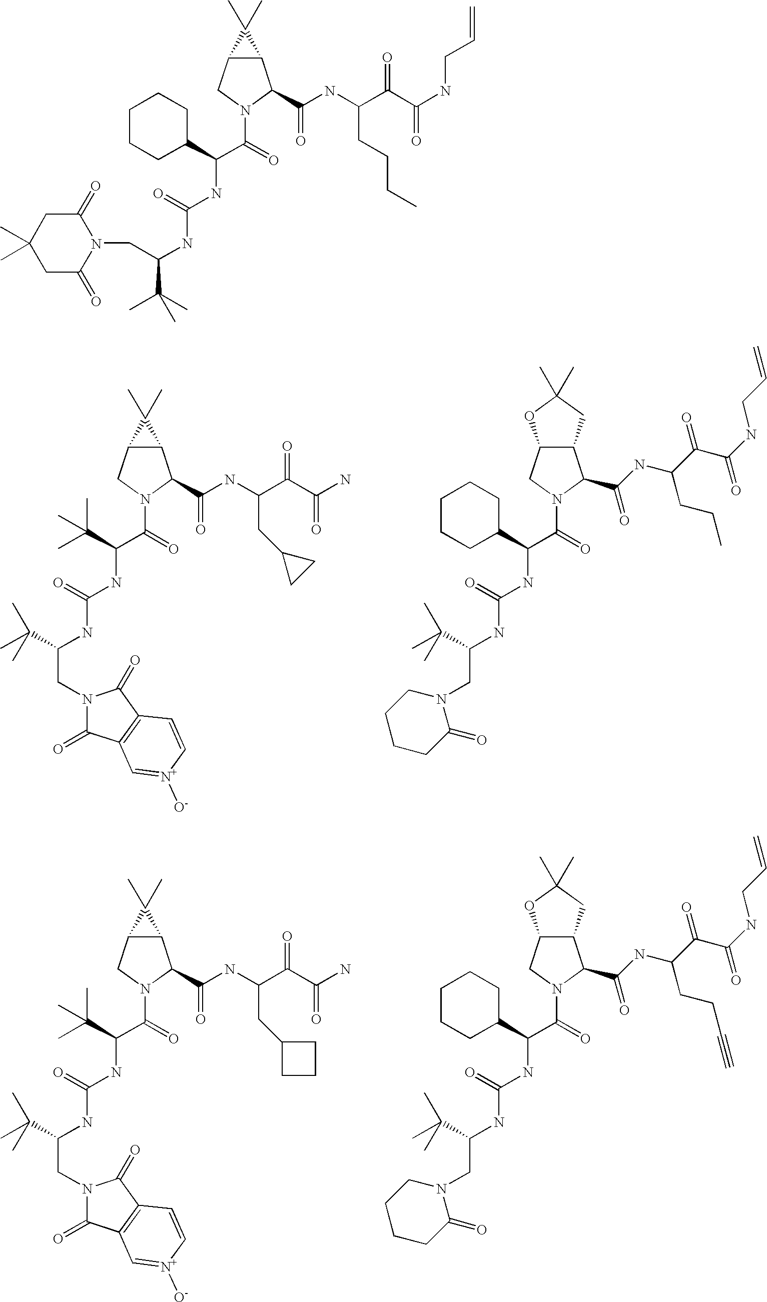 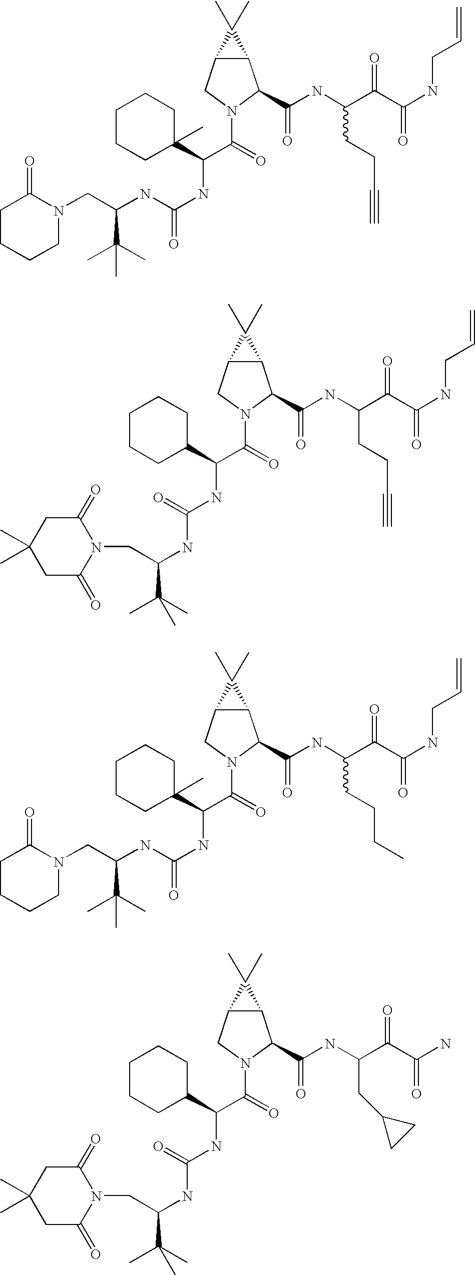 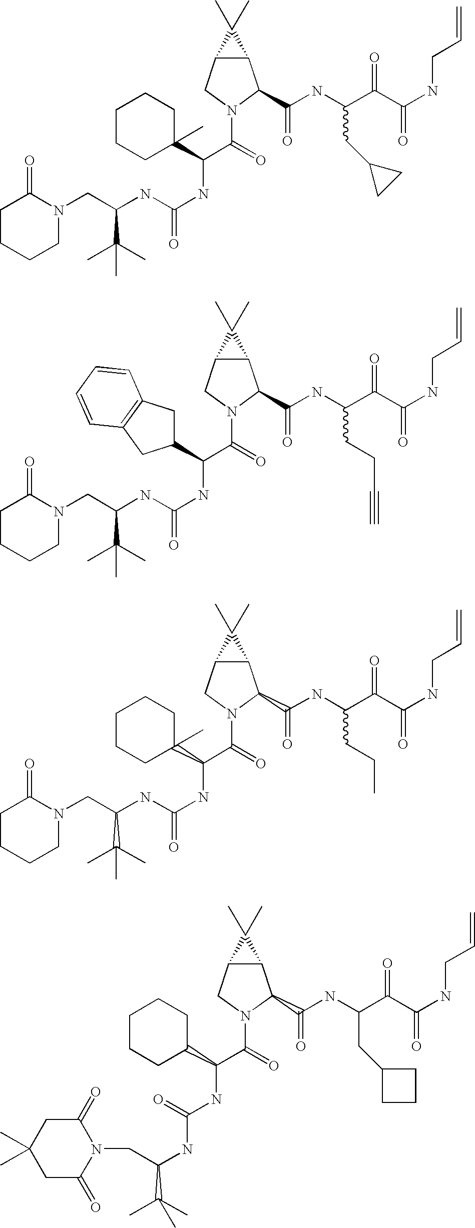 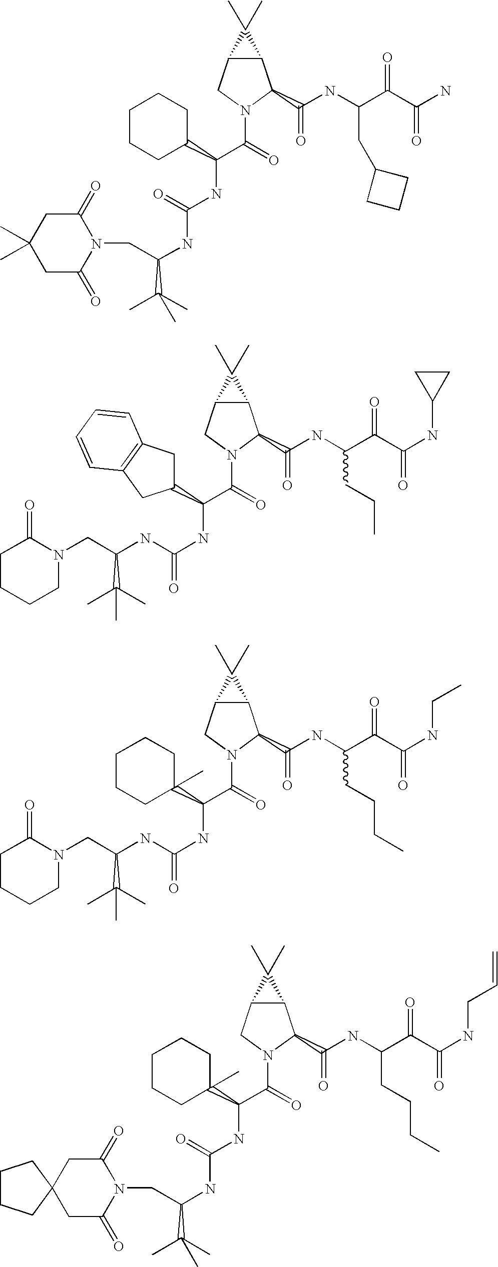 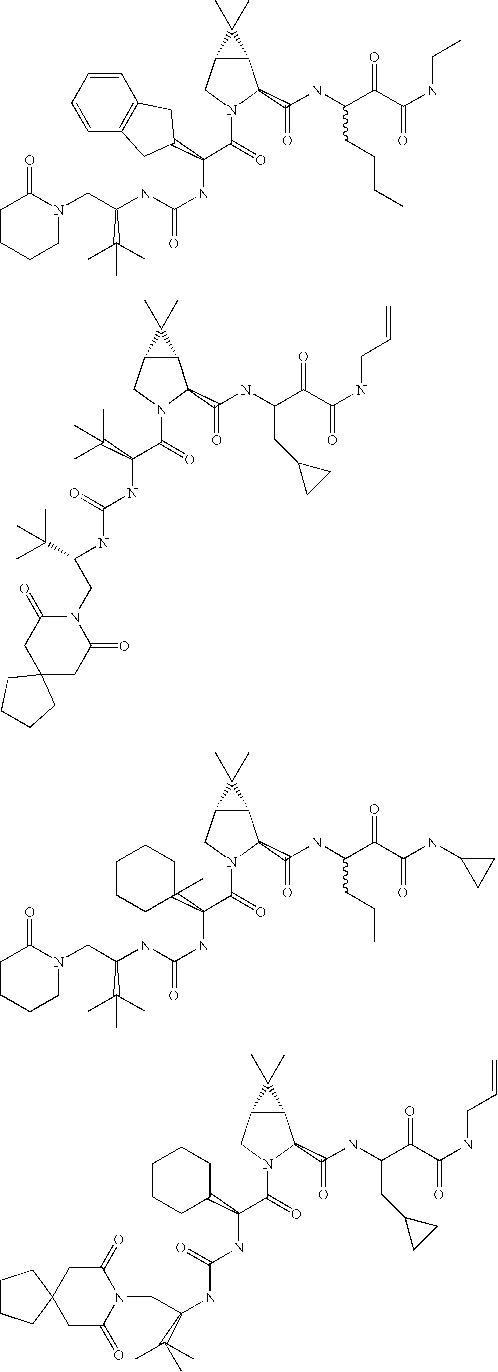 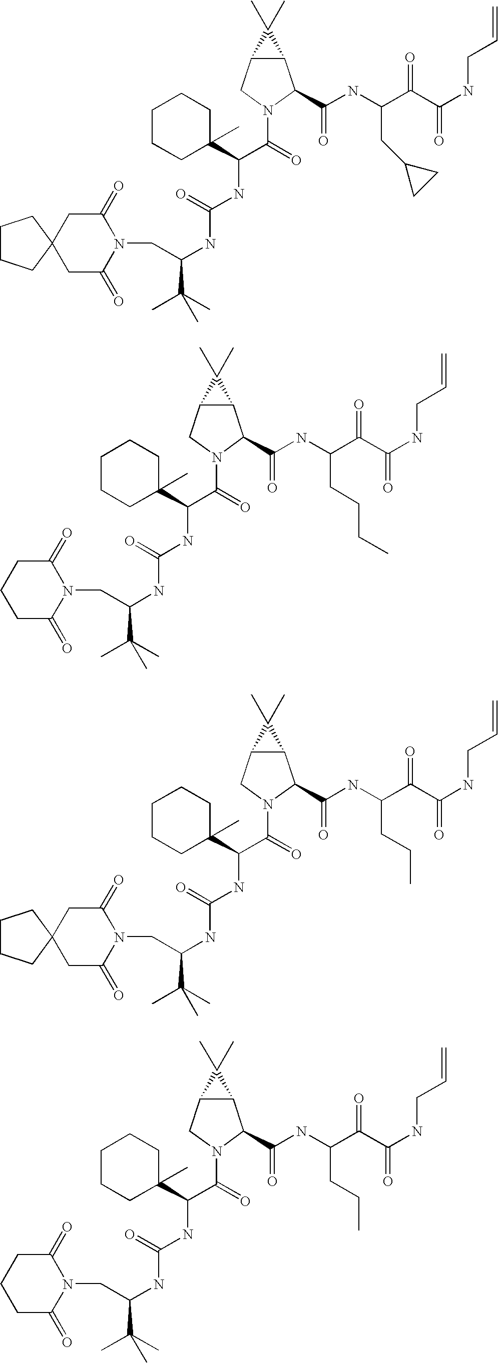 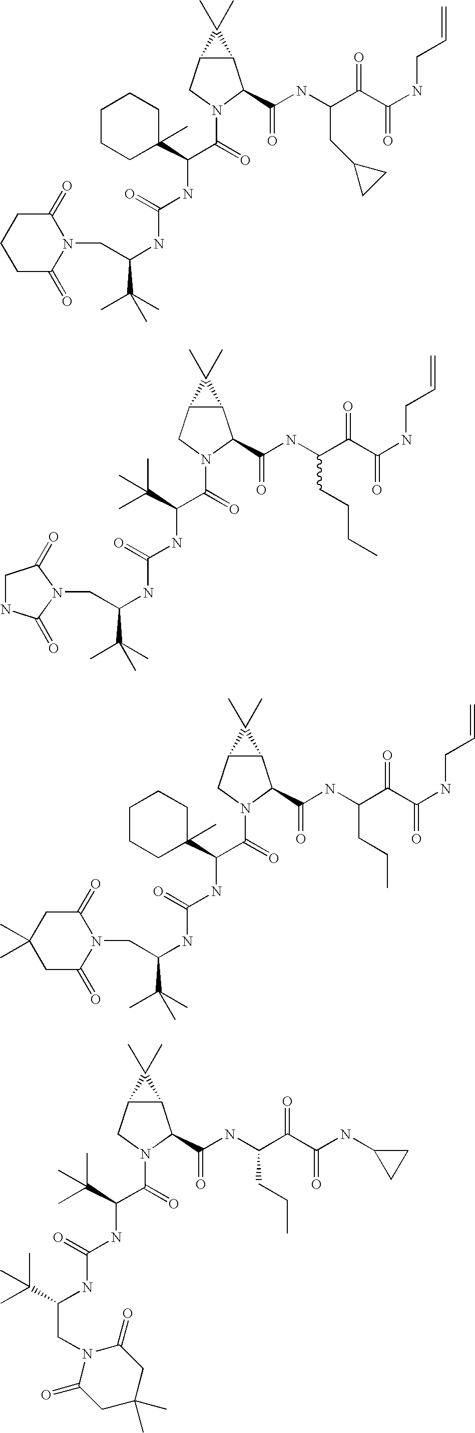 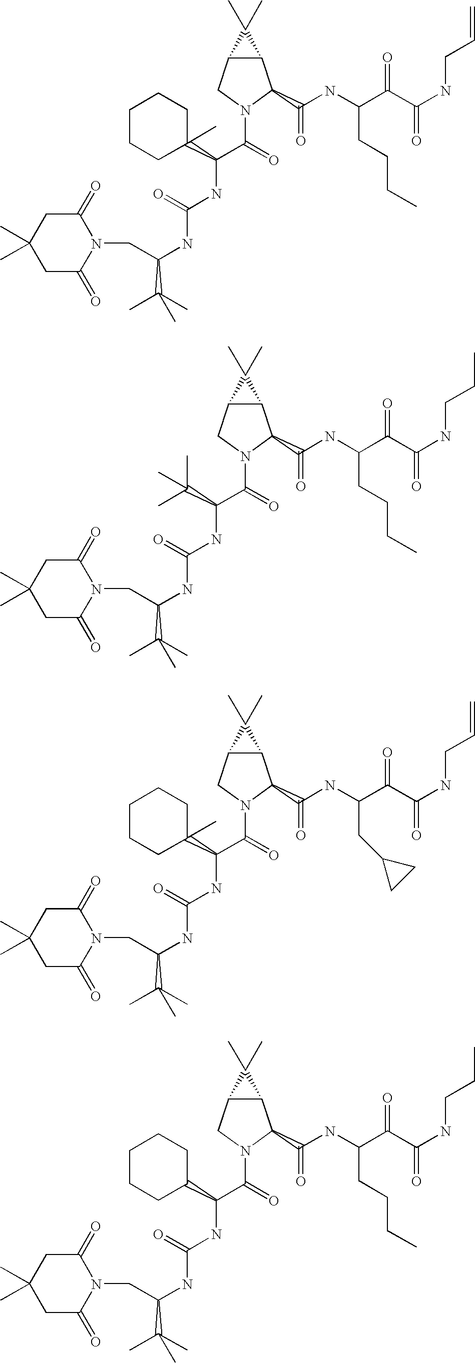 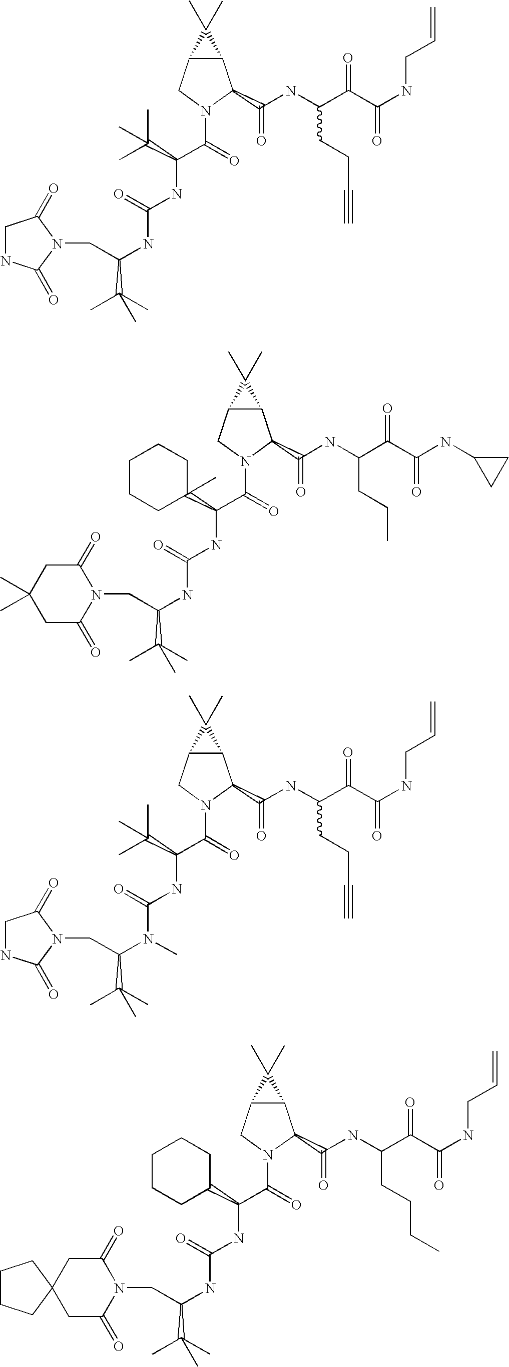 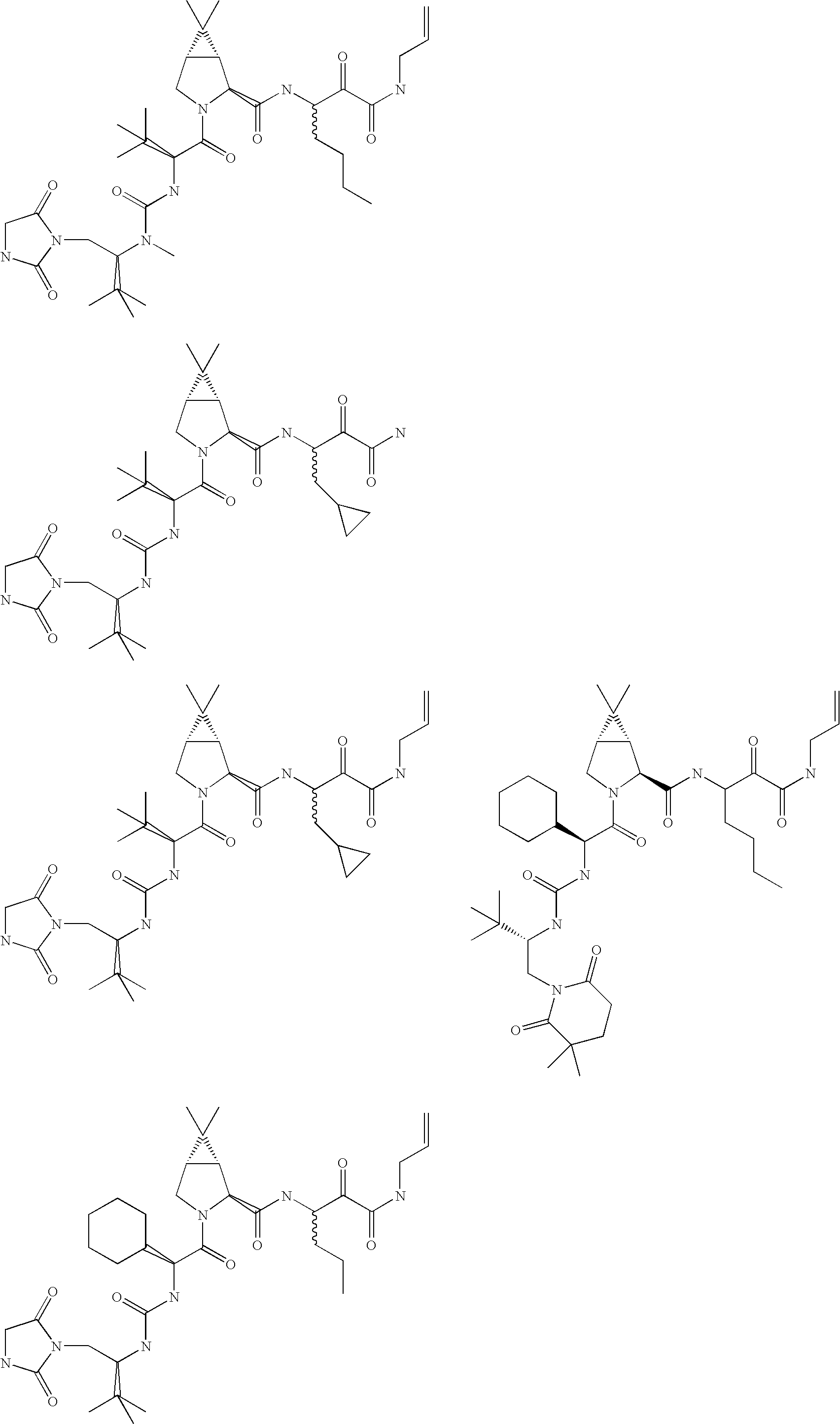 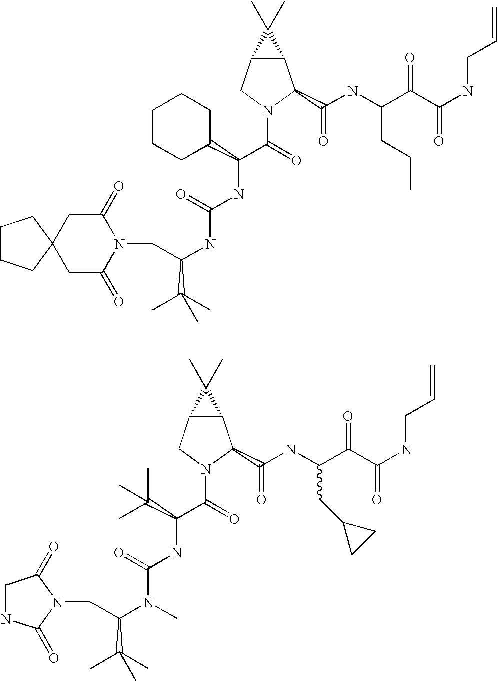
or a pharmaceutically acceptable salt, solvate or ester thereof.

Compounds of formula XIV are disclosed in U.S. Patent Application Ser. No. 11/064,673 filed Feb. 24, 2005, and the corresponding PCT publication WO2005/087731, published Sep. 22, 2005, both of which are incorporated herein by reference.

Non-limiting examples of certain compounds disclosed in U.S. patent application Ser. No.11/064,673 and WO 2005/087731 are: 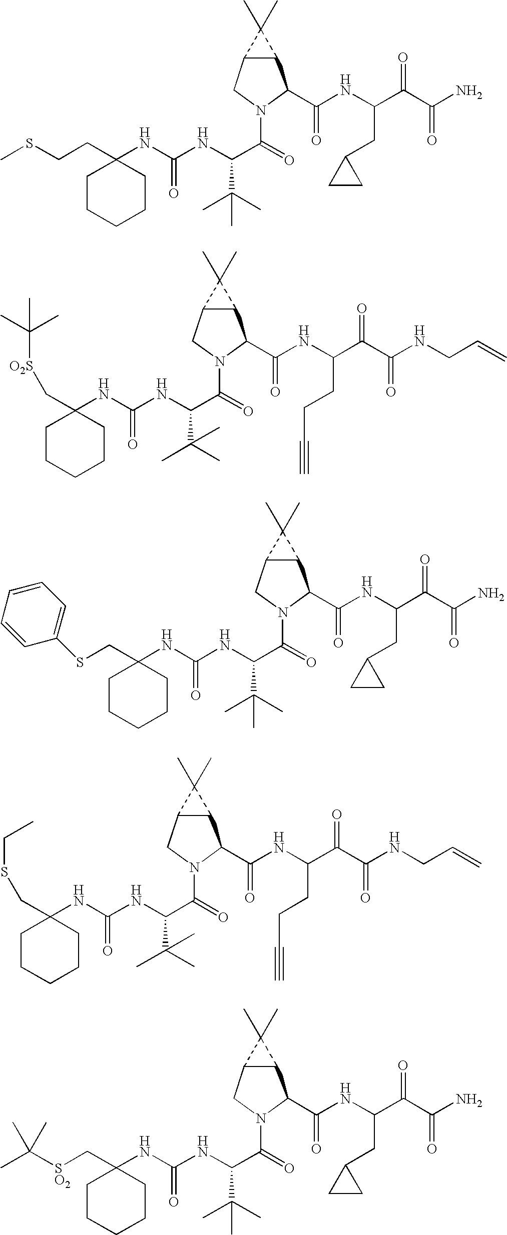 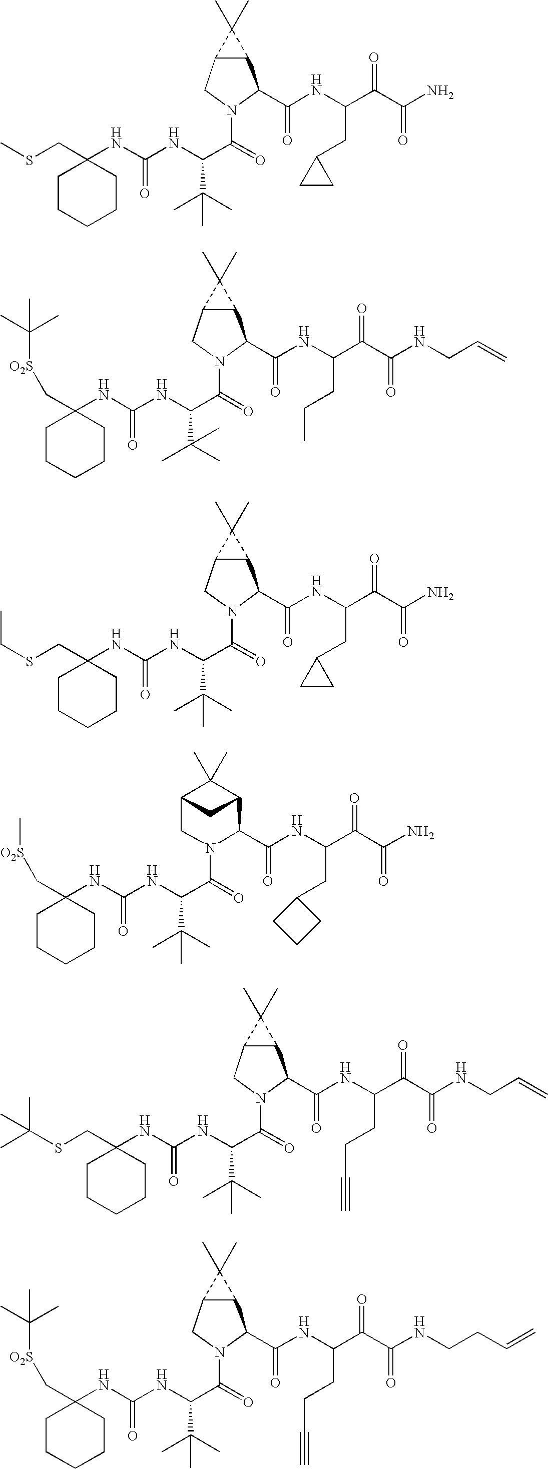 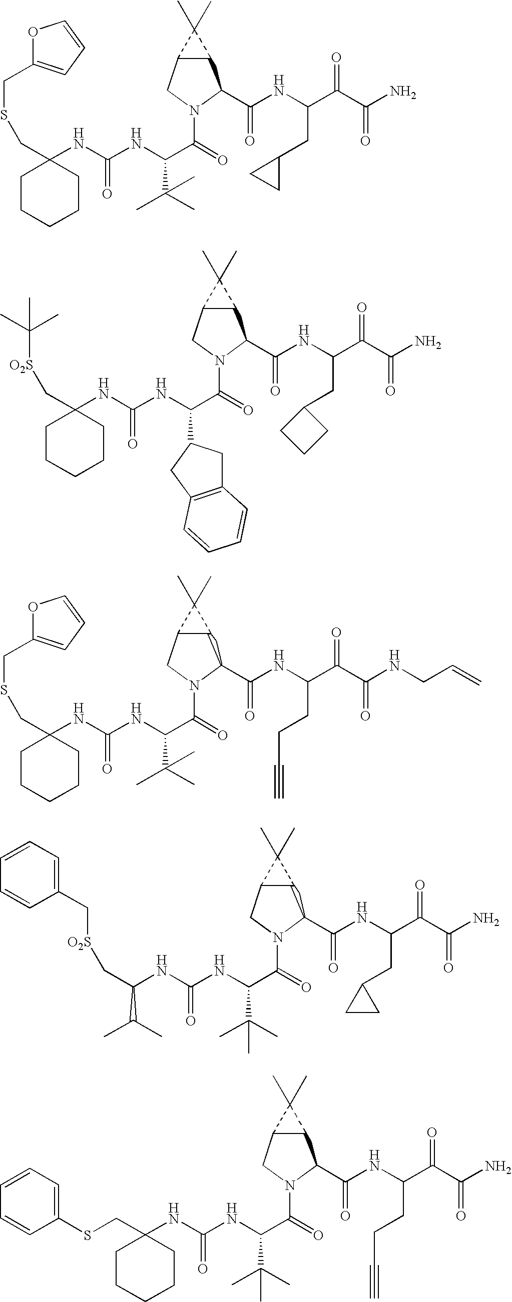 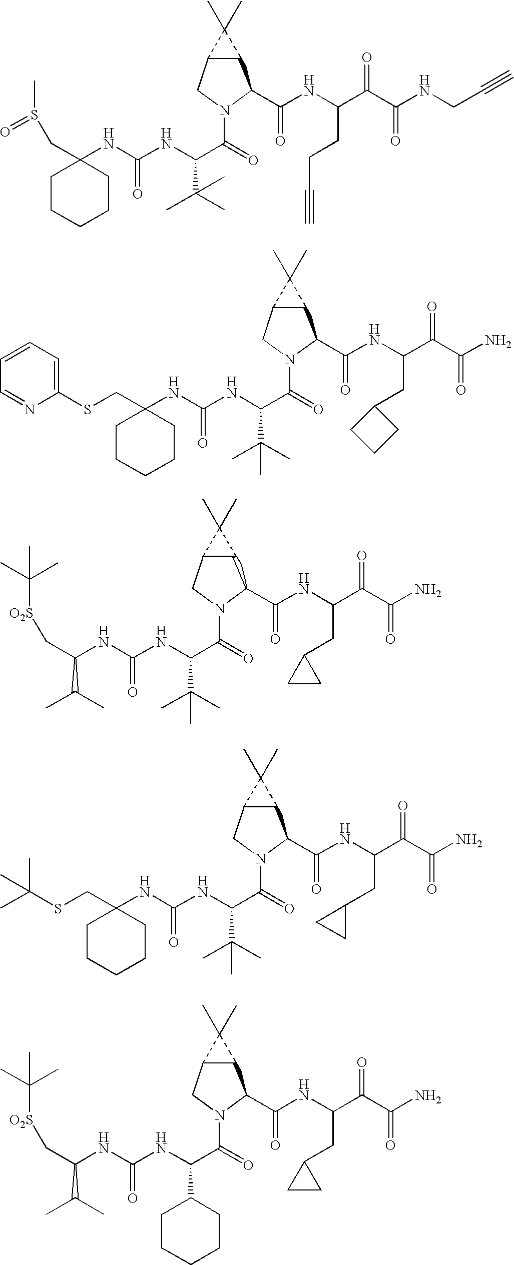 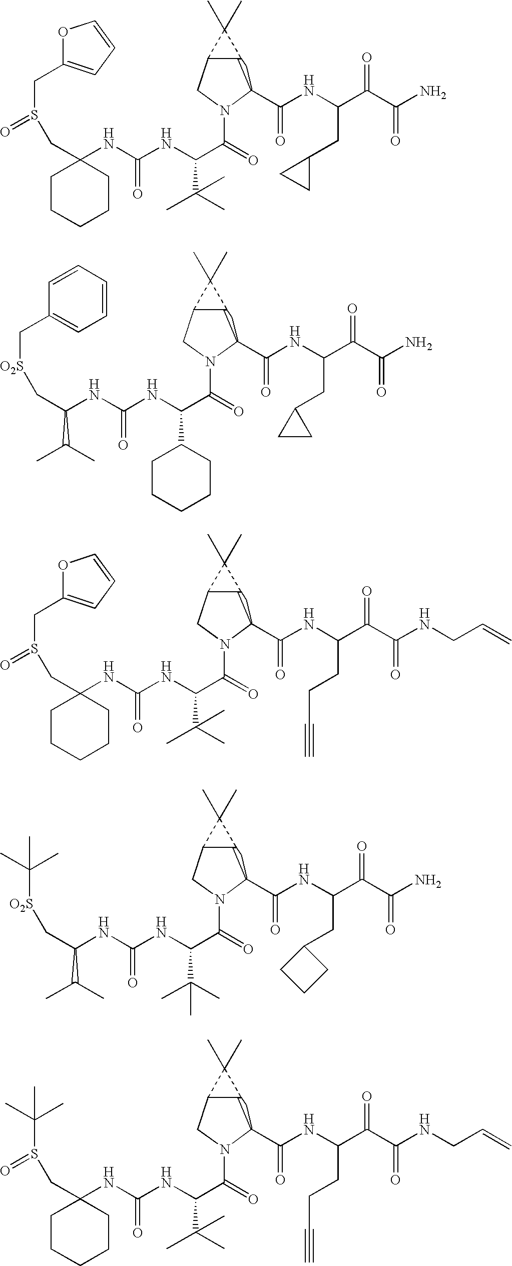 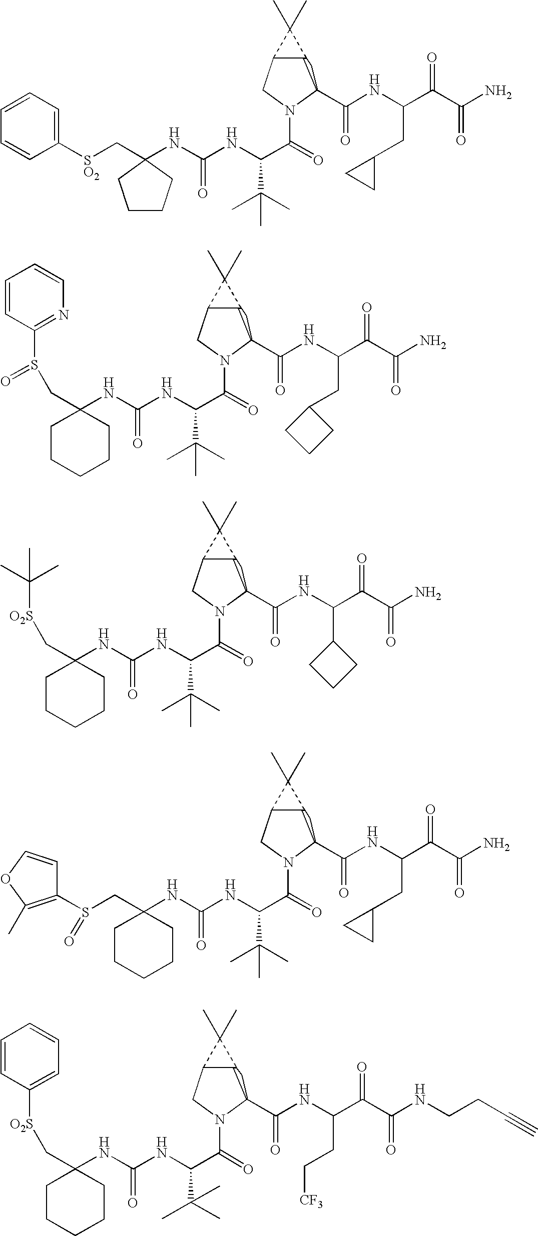 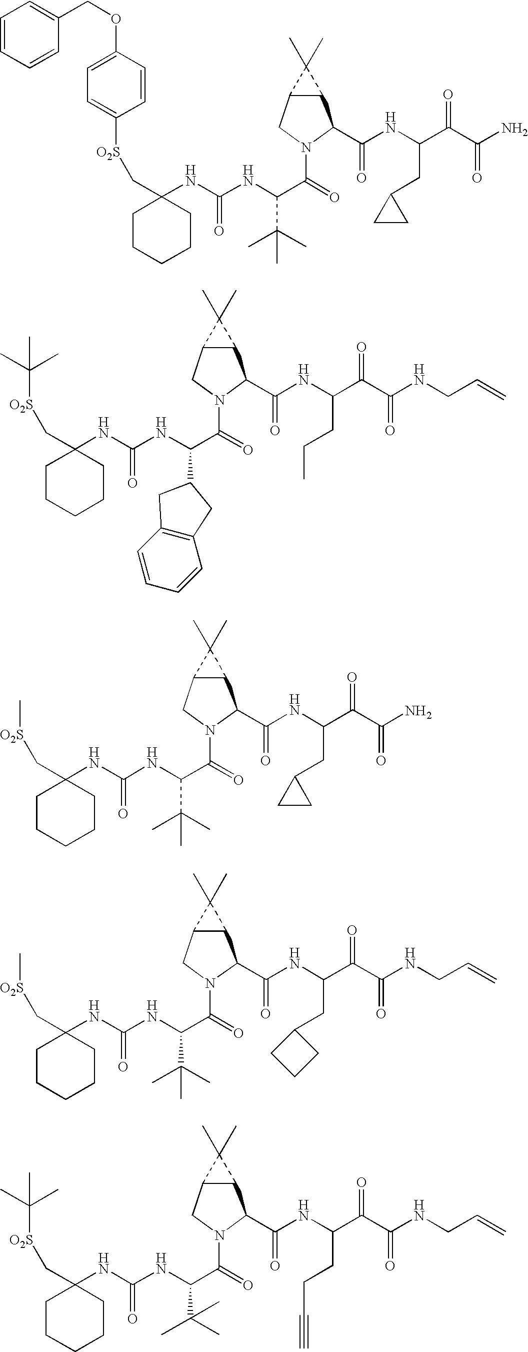 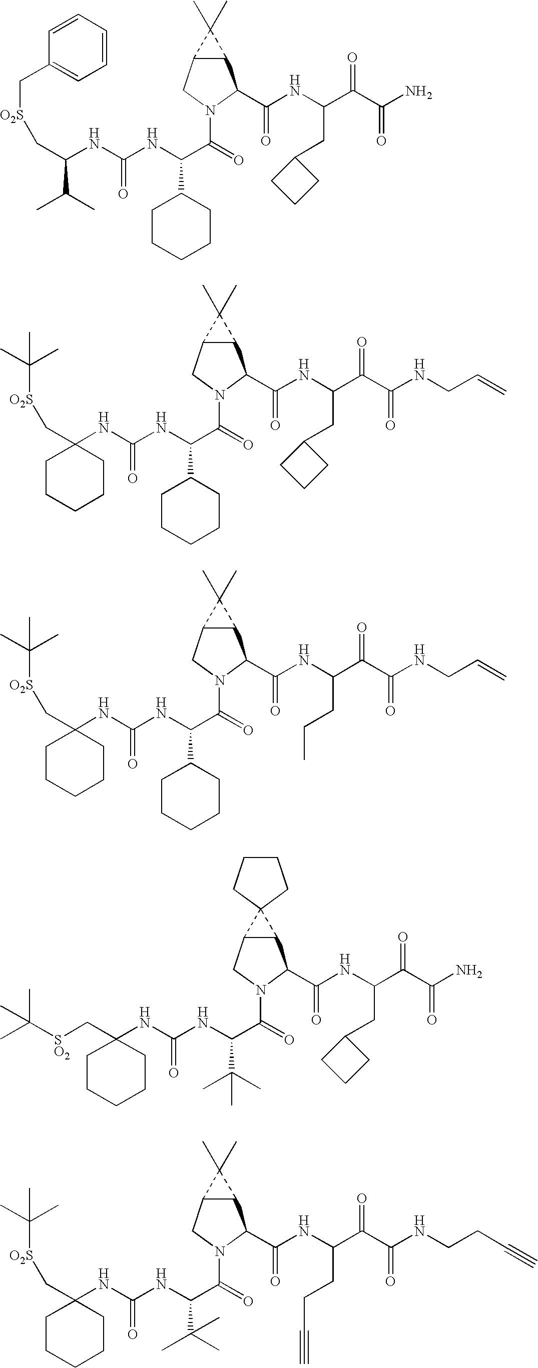 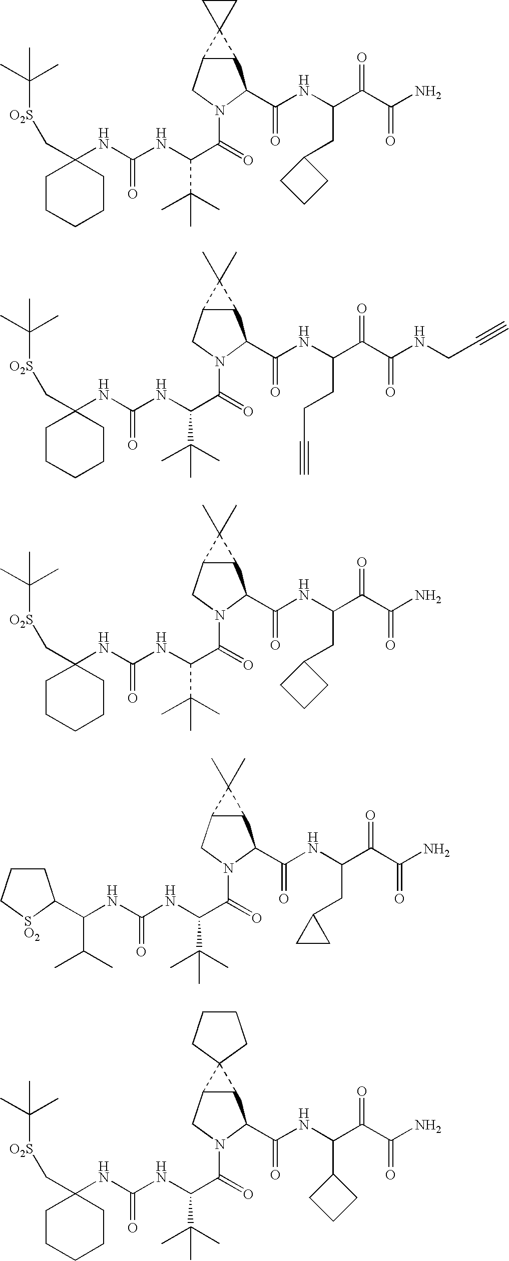 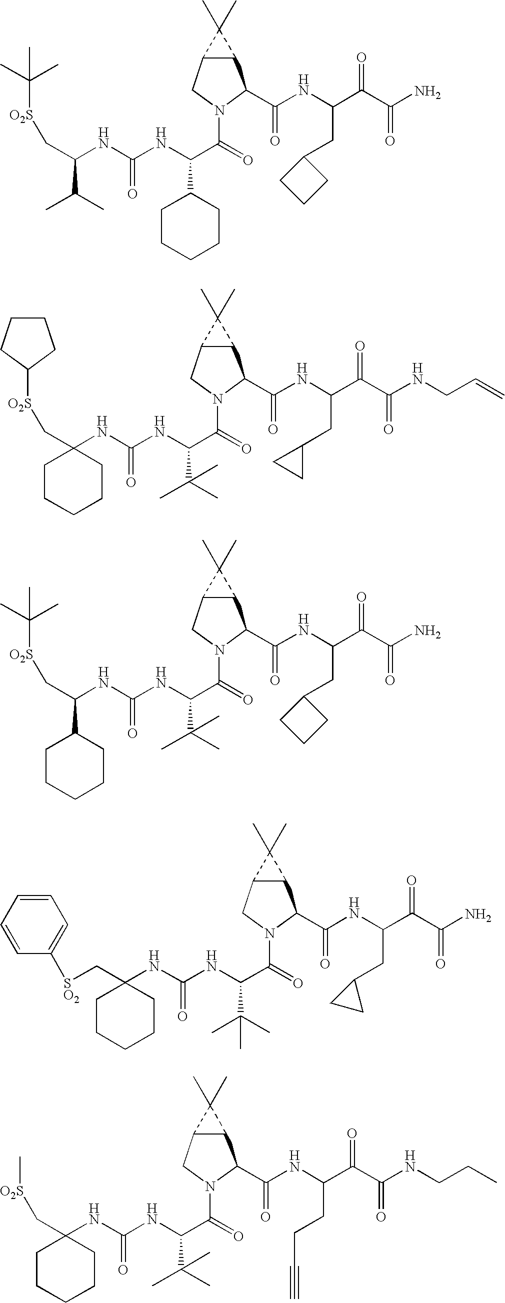 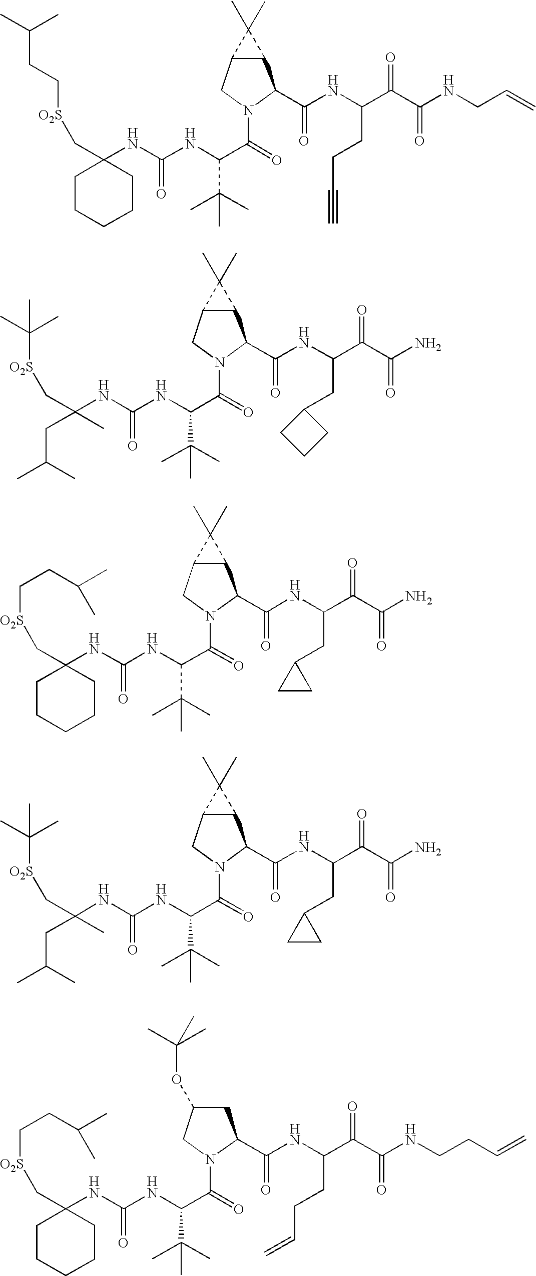 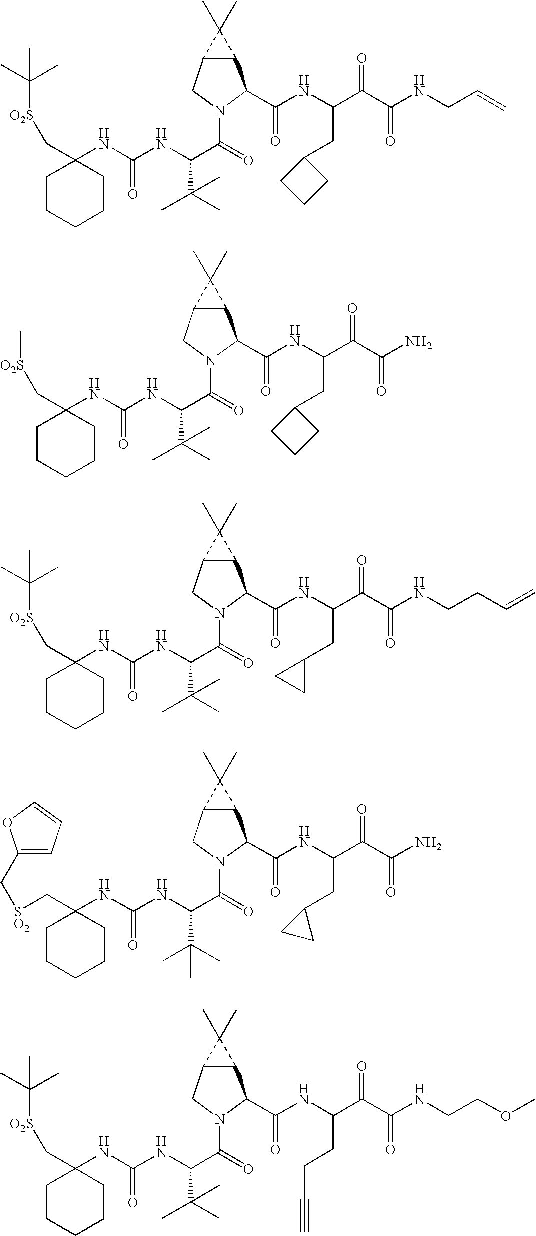 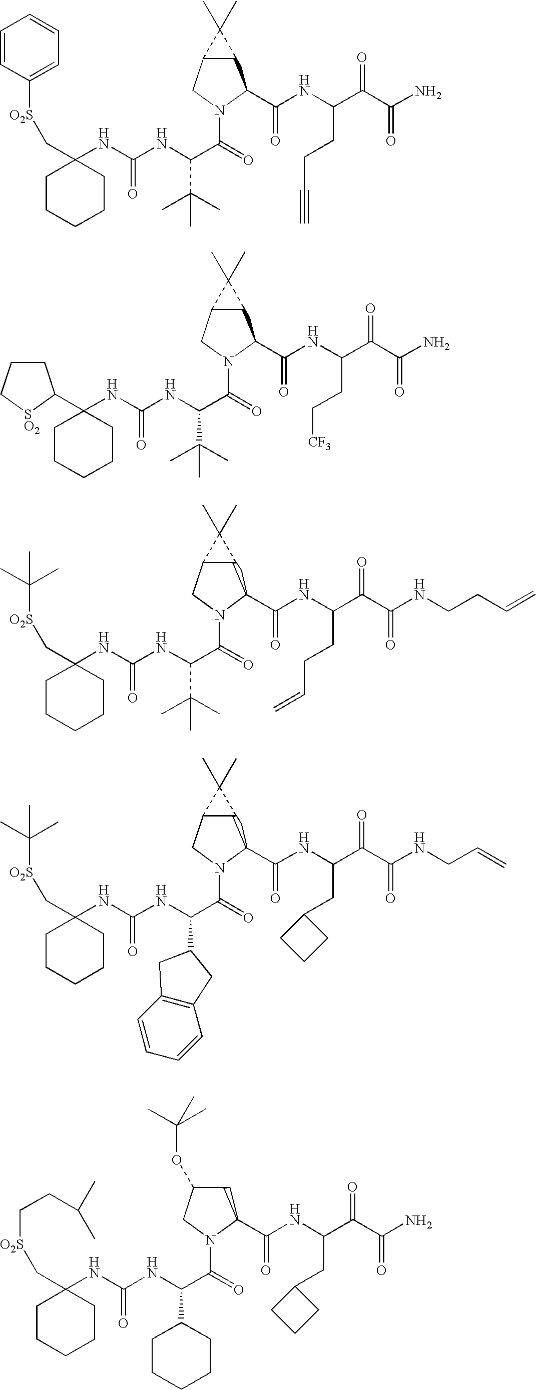 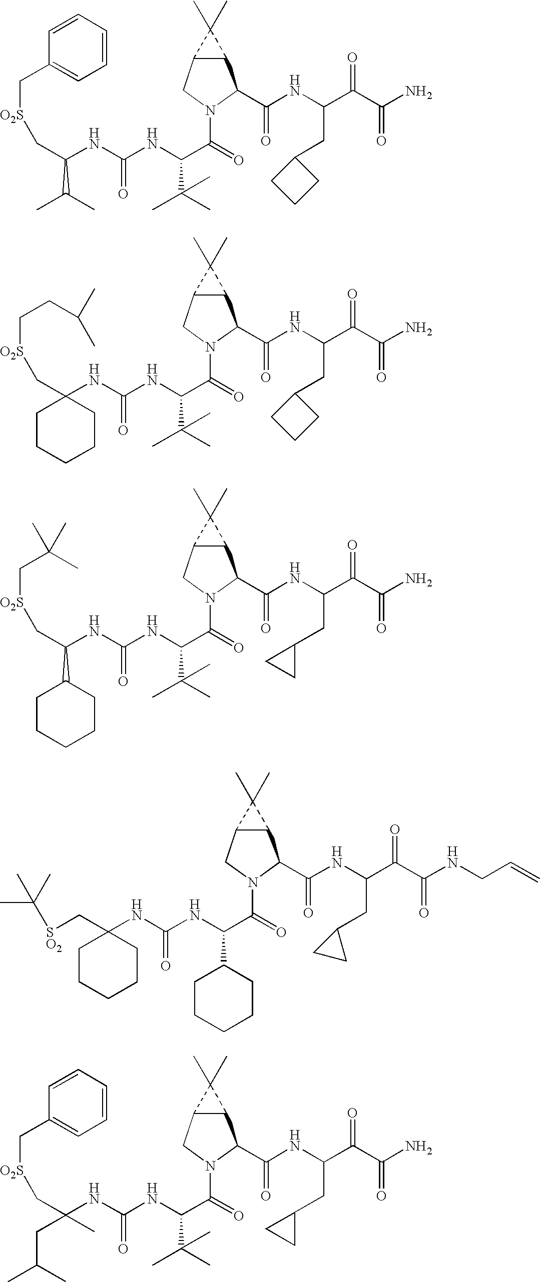 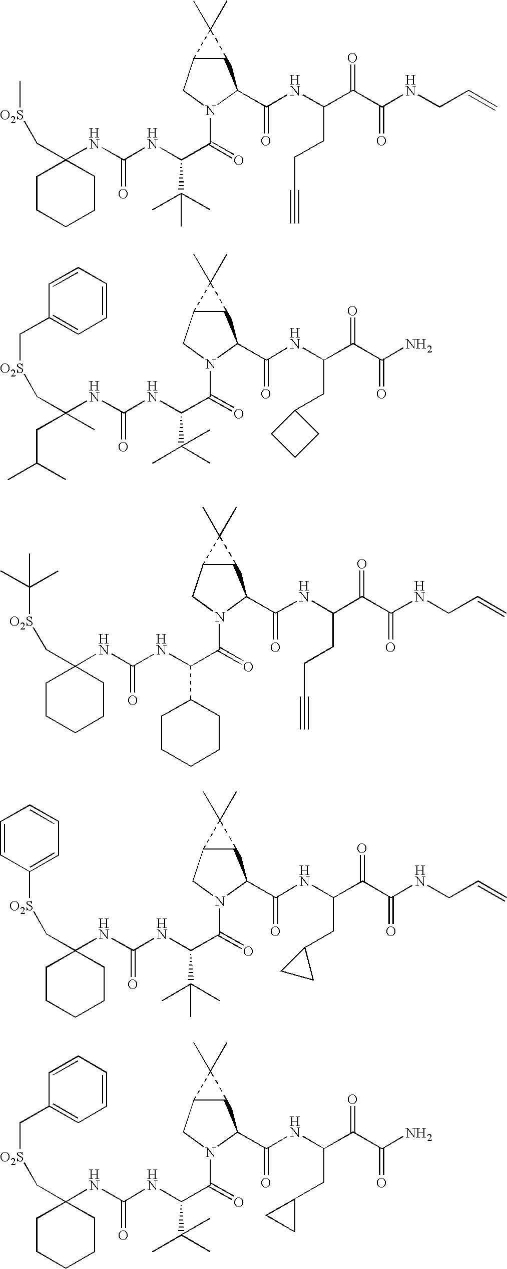 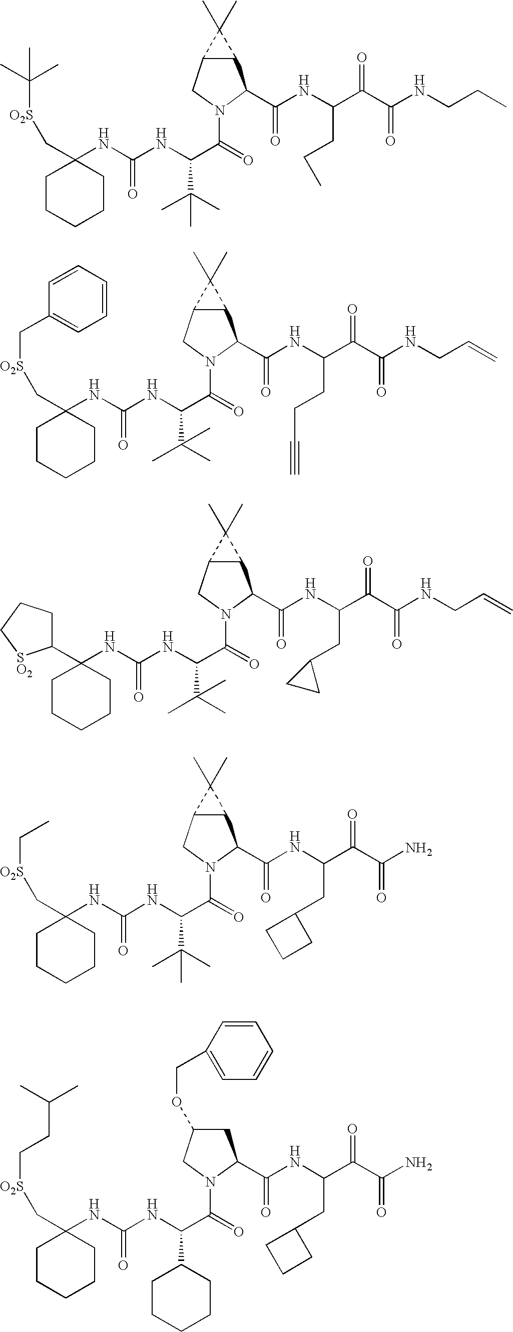 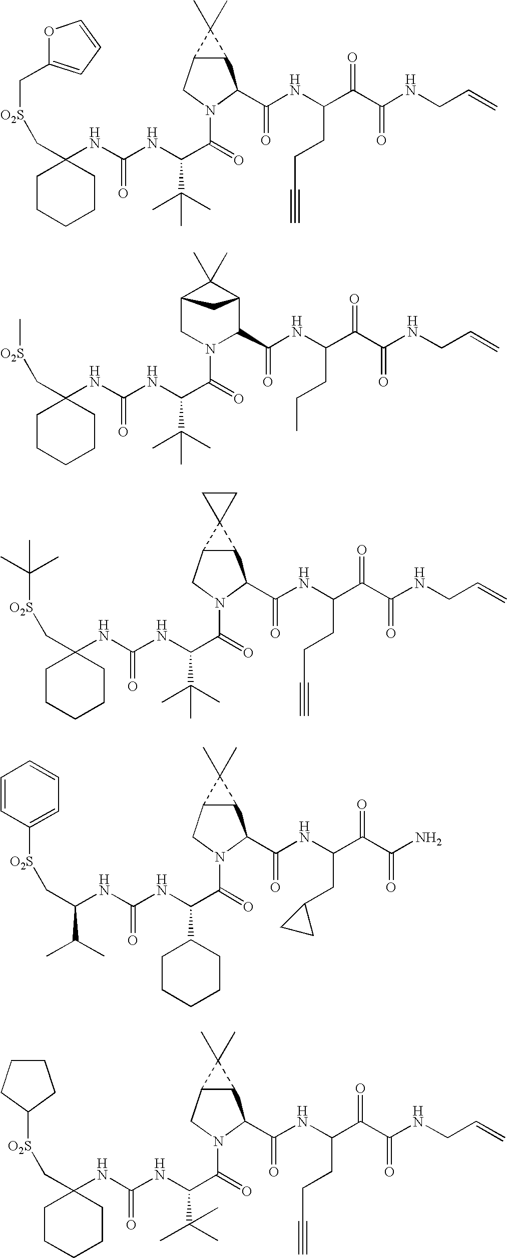 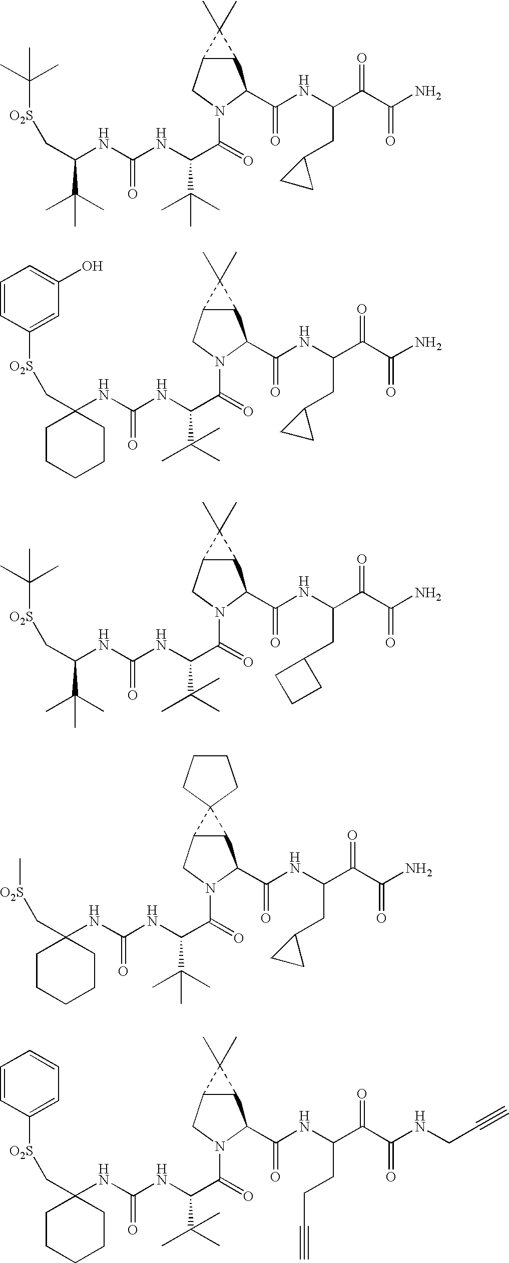 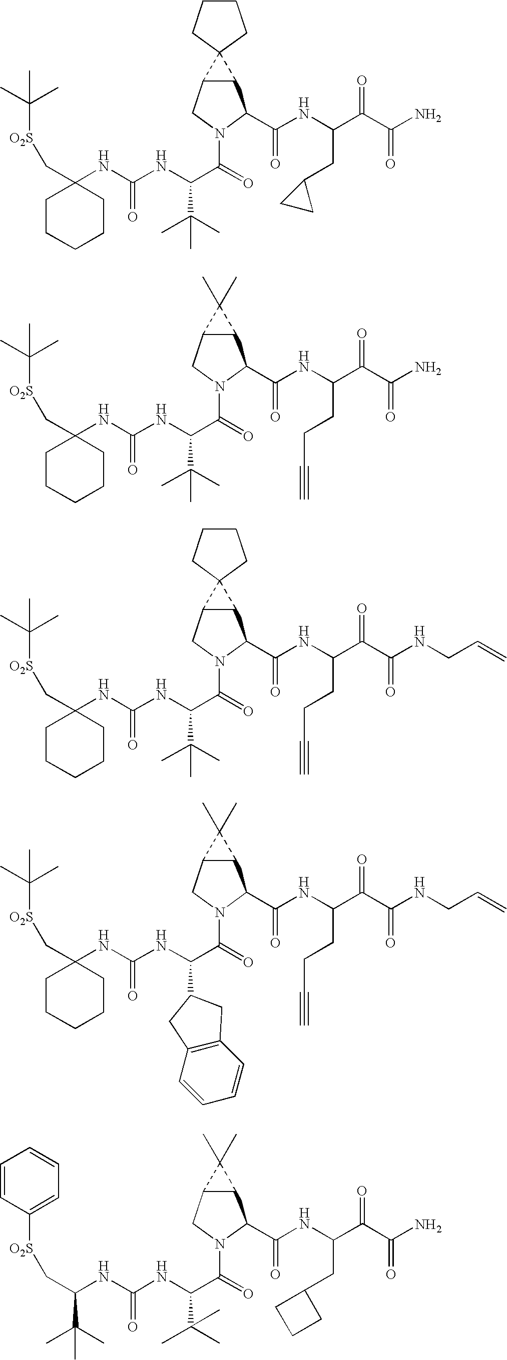 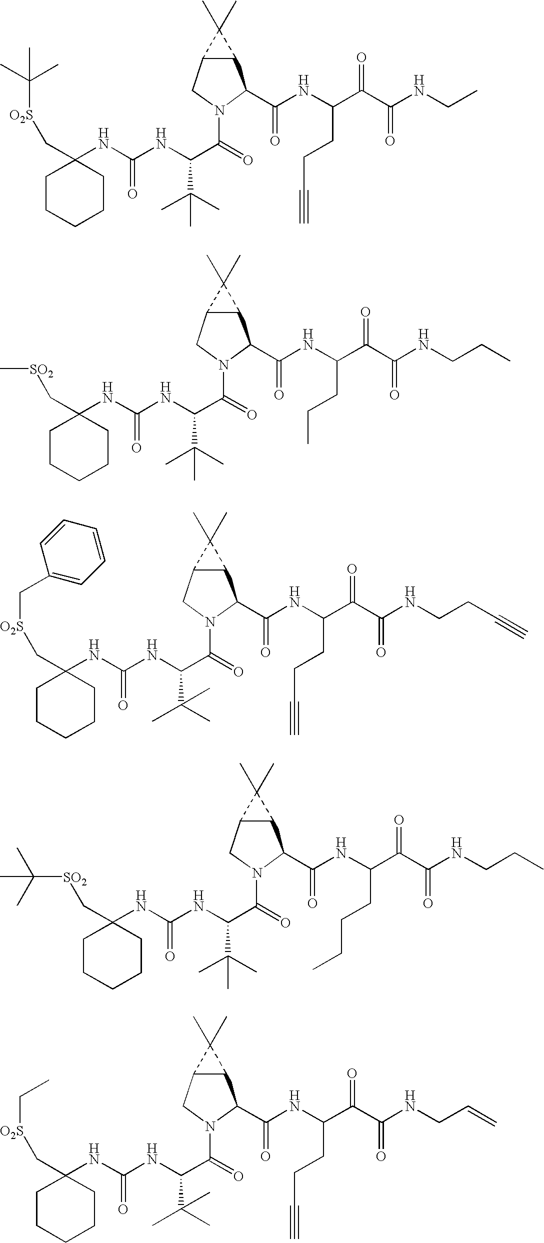 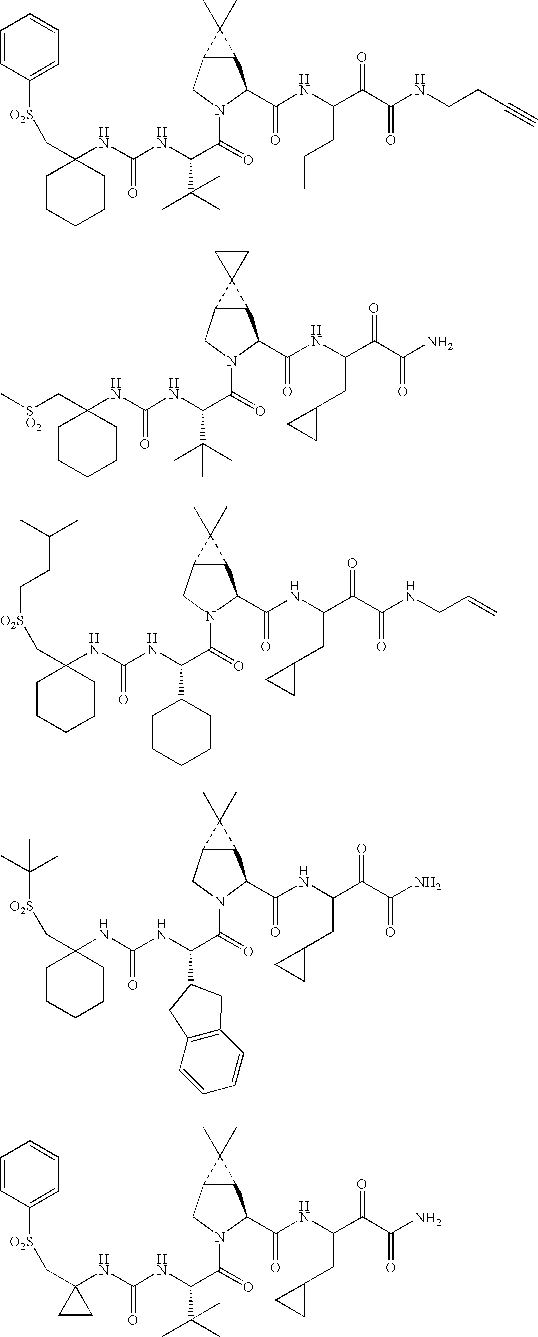 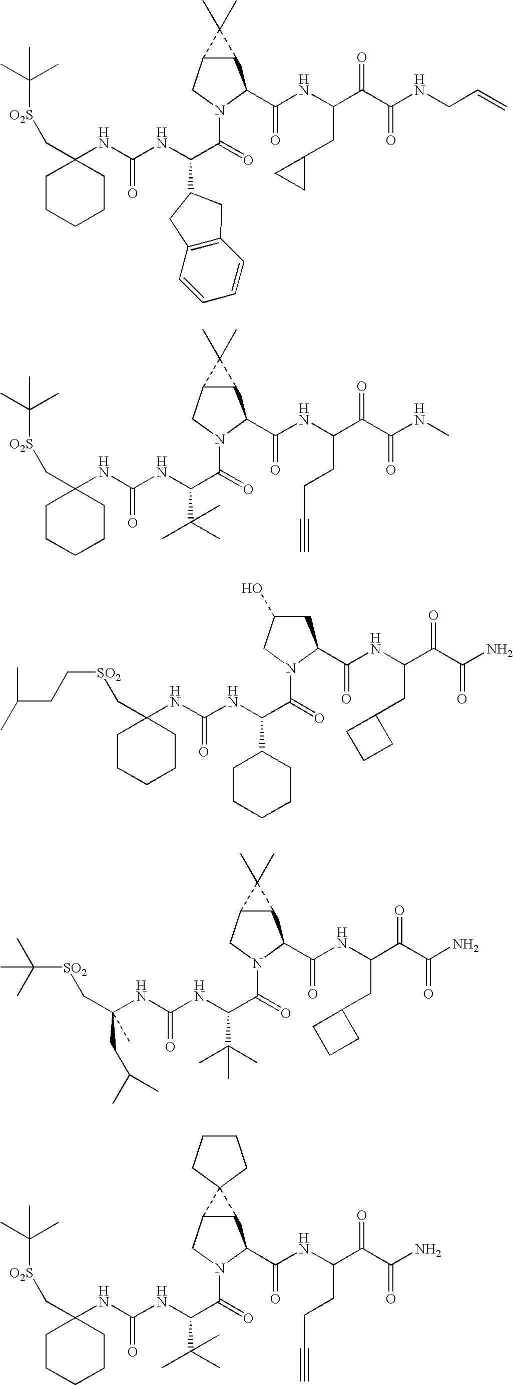 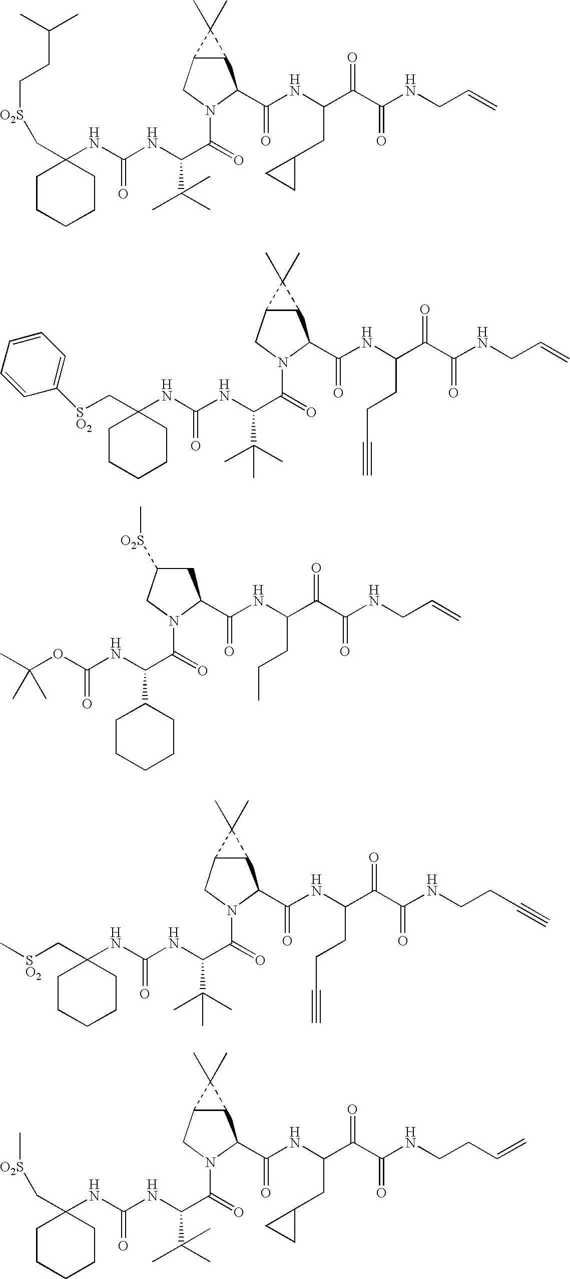 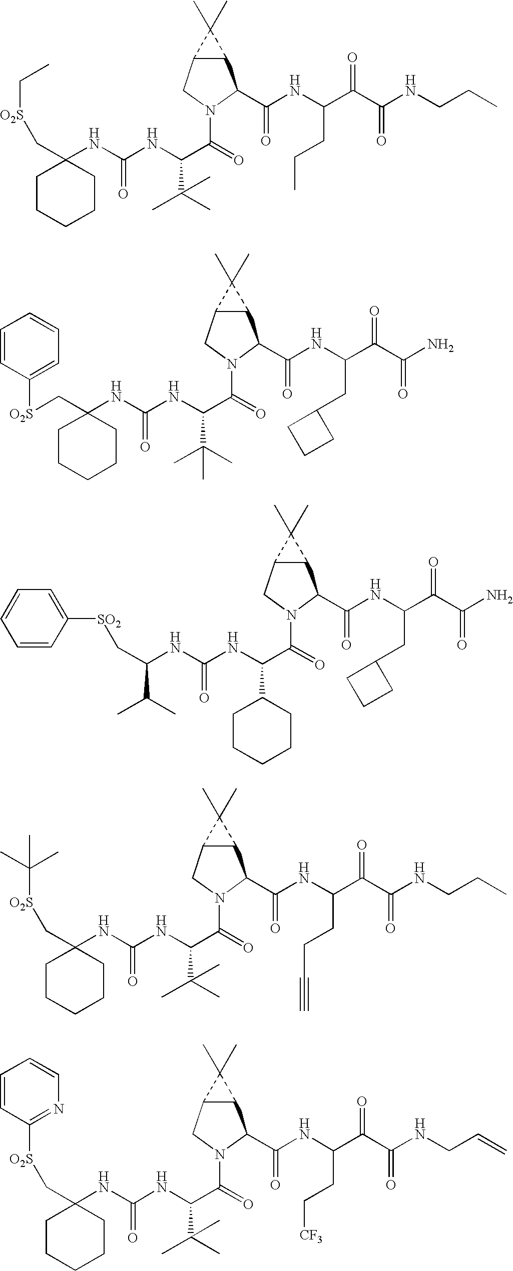 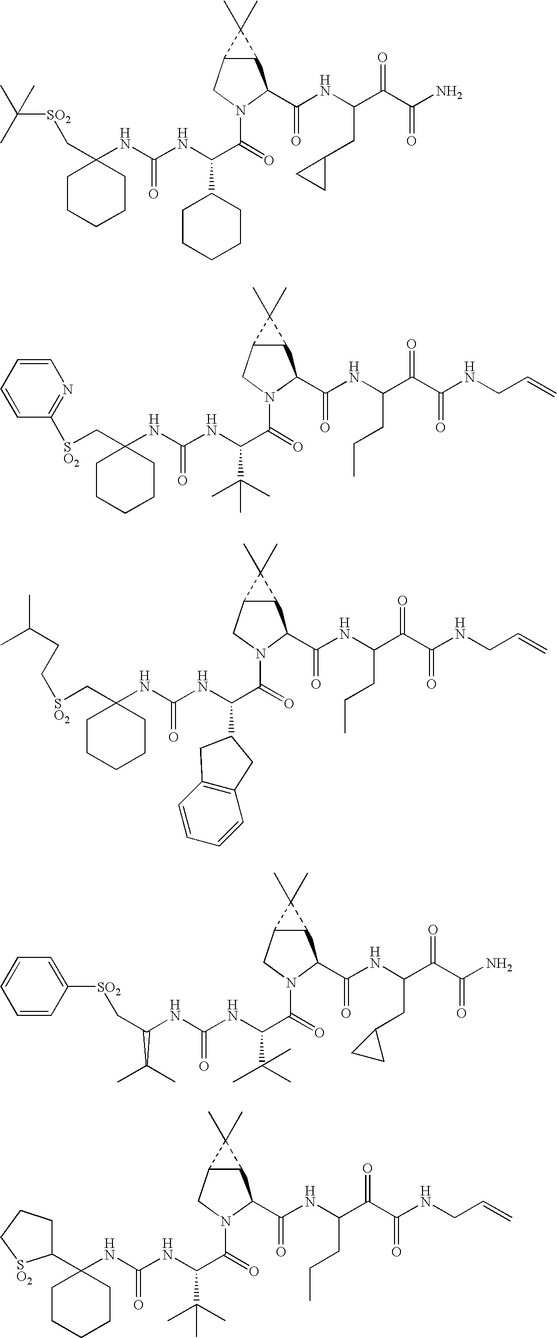 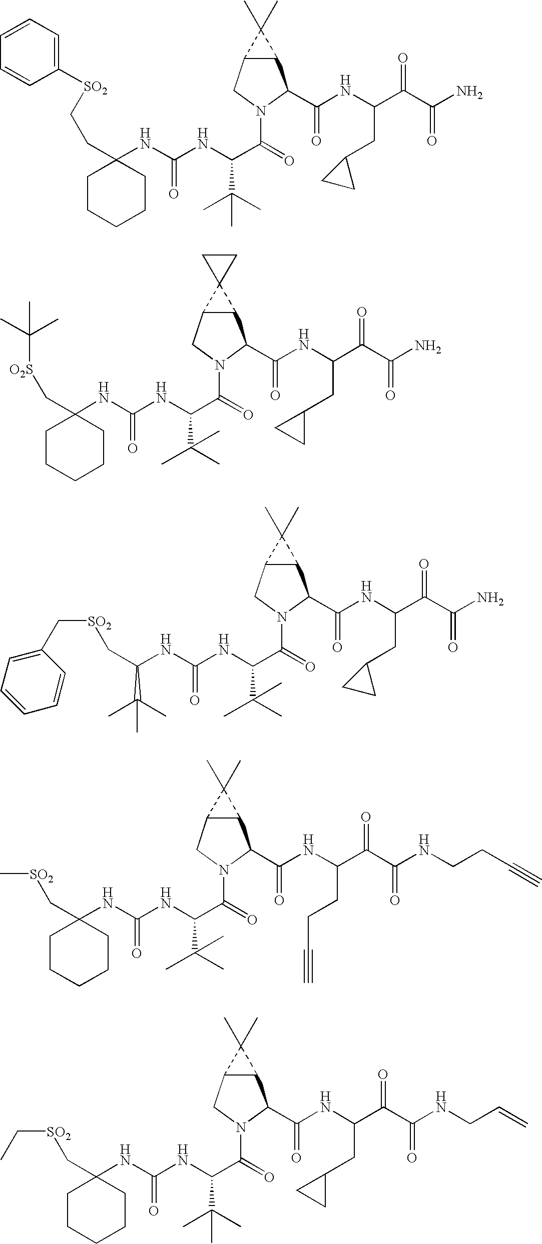 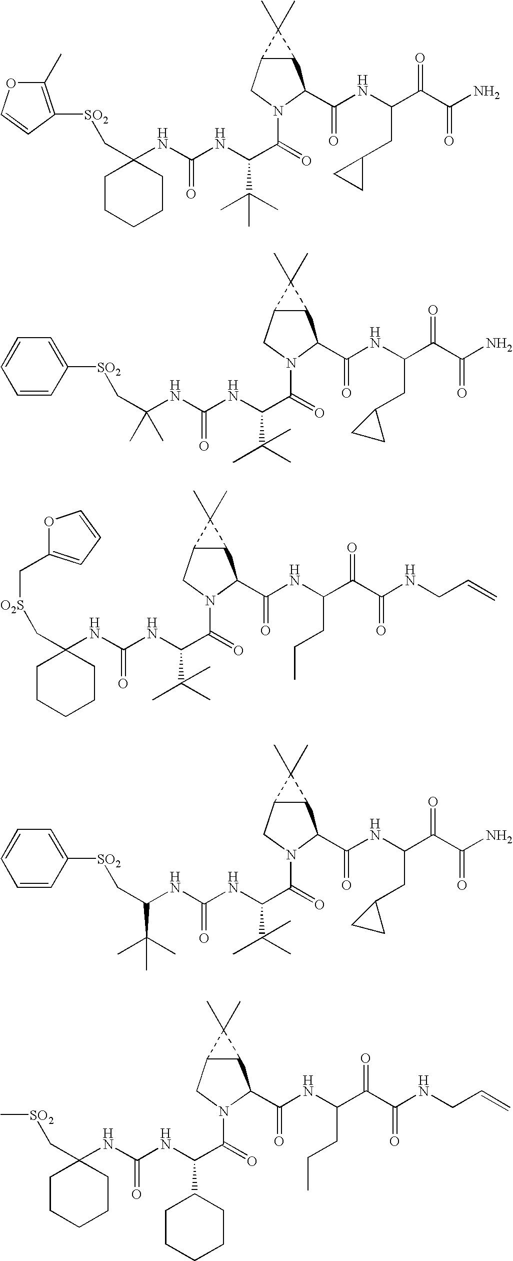 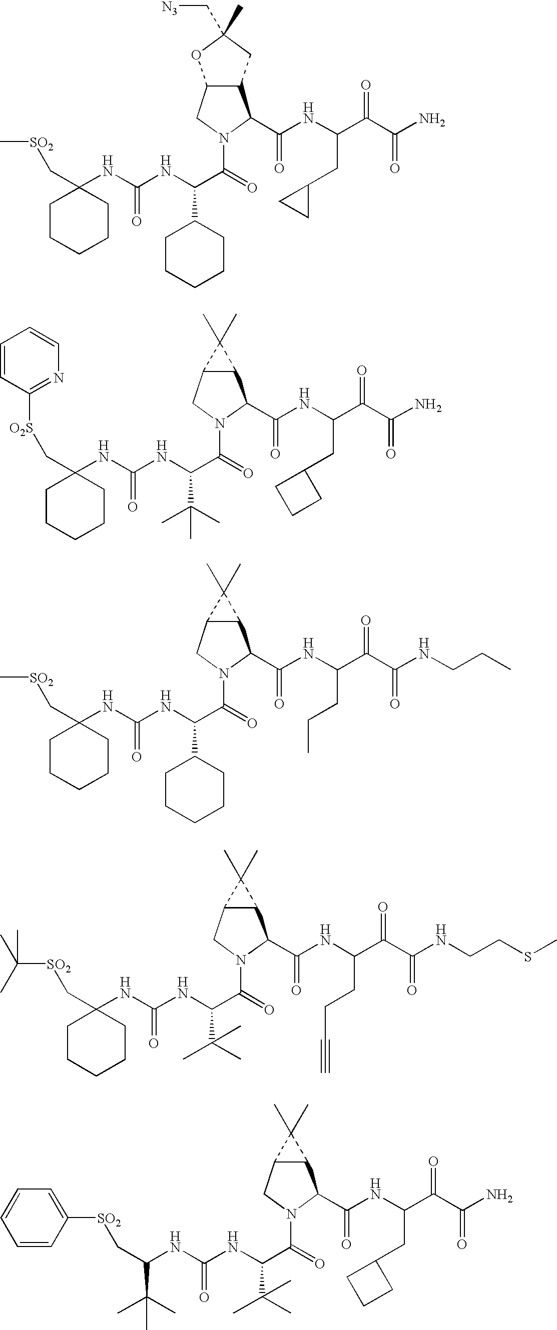 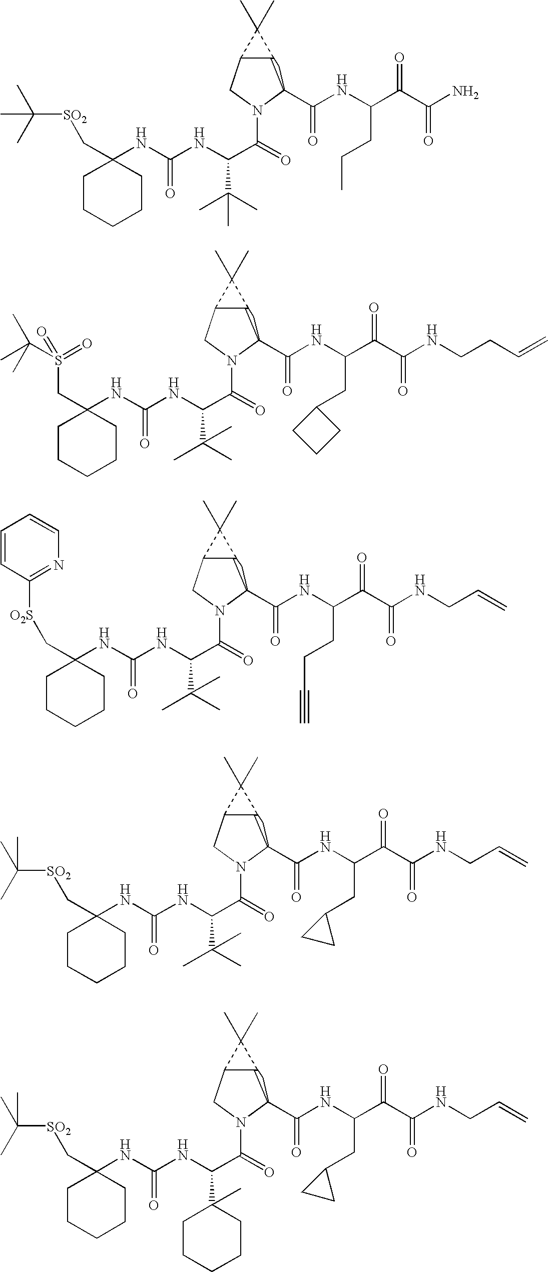 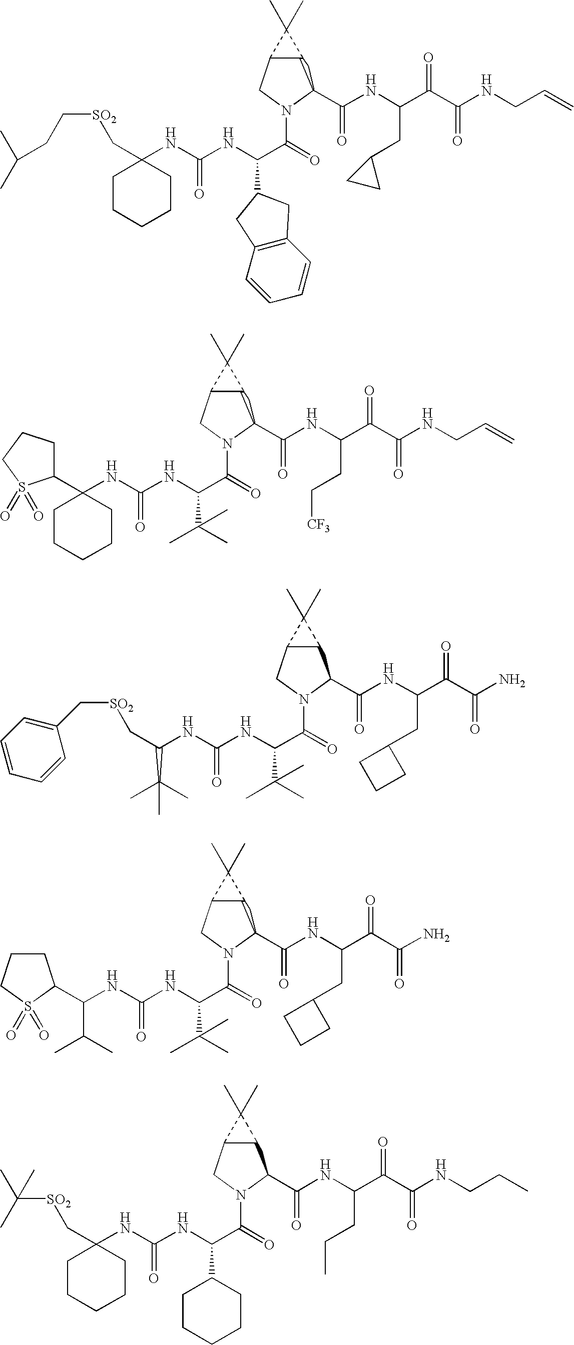 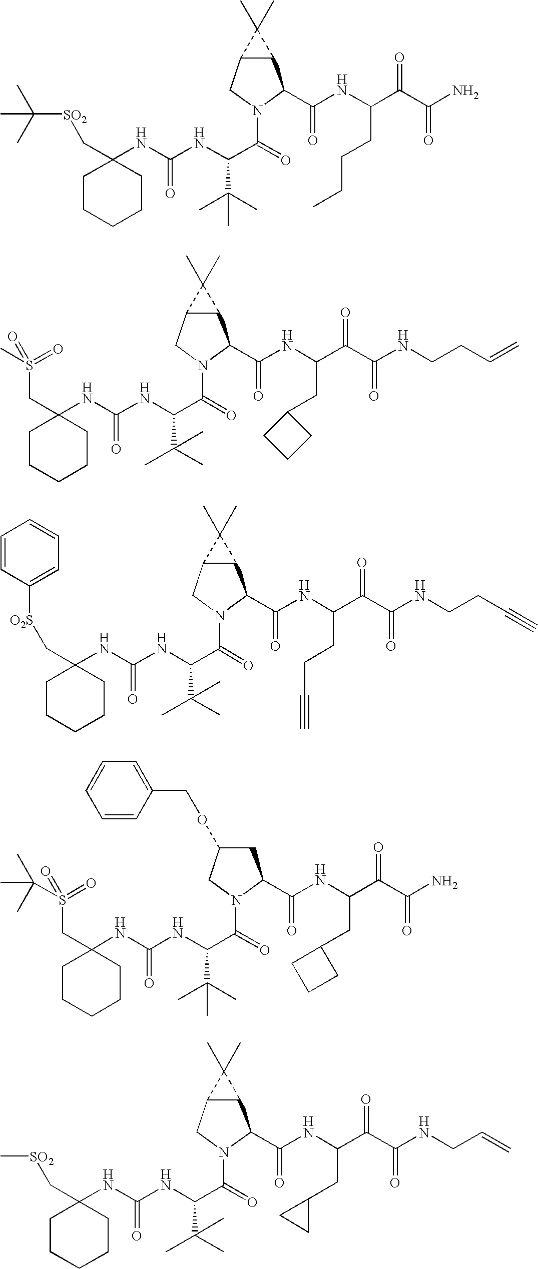 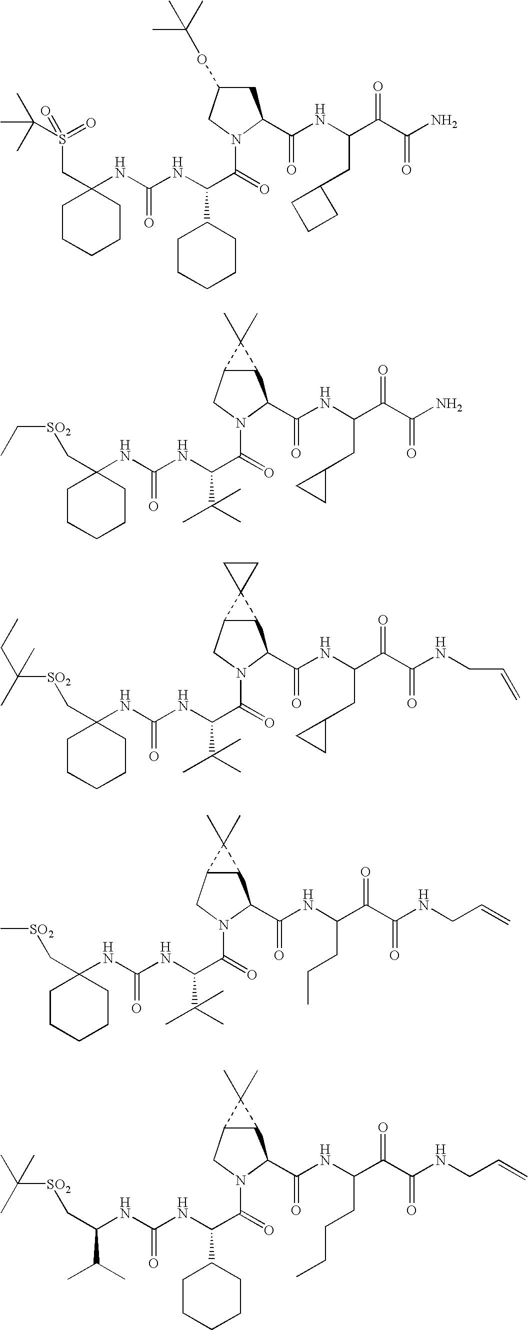 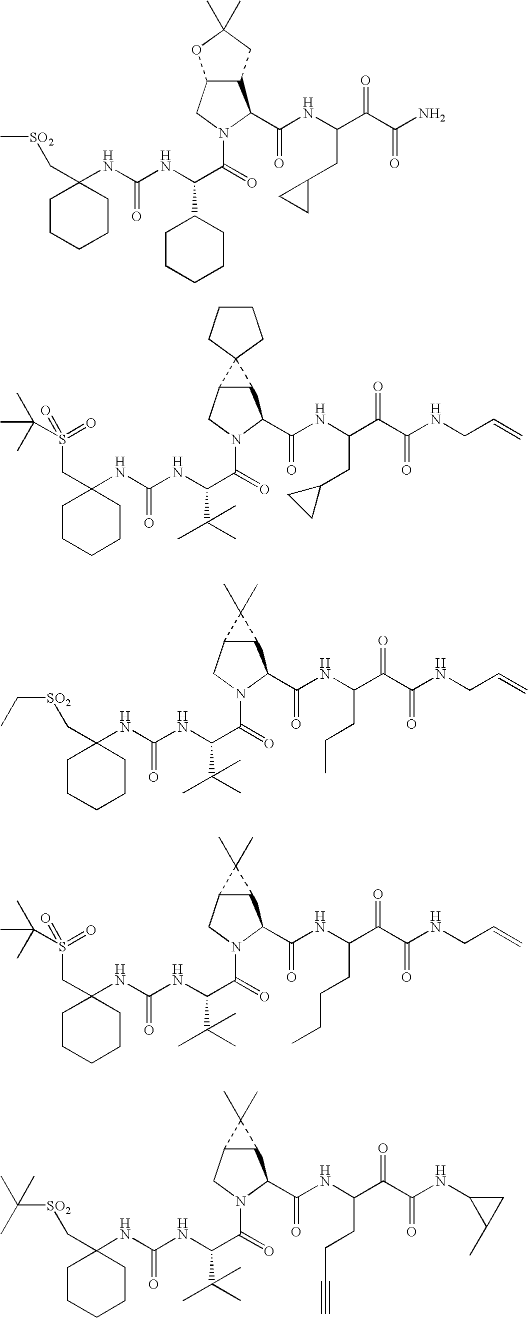 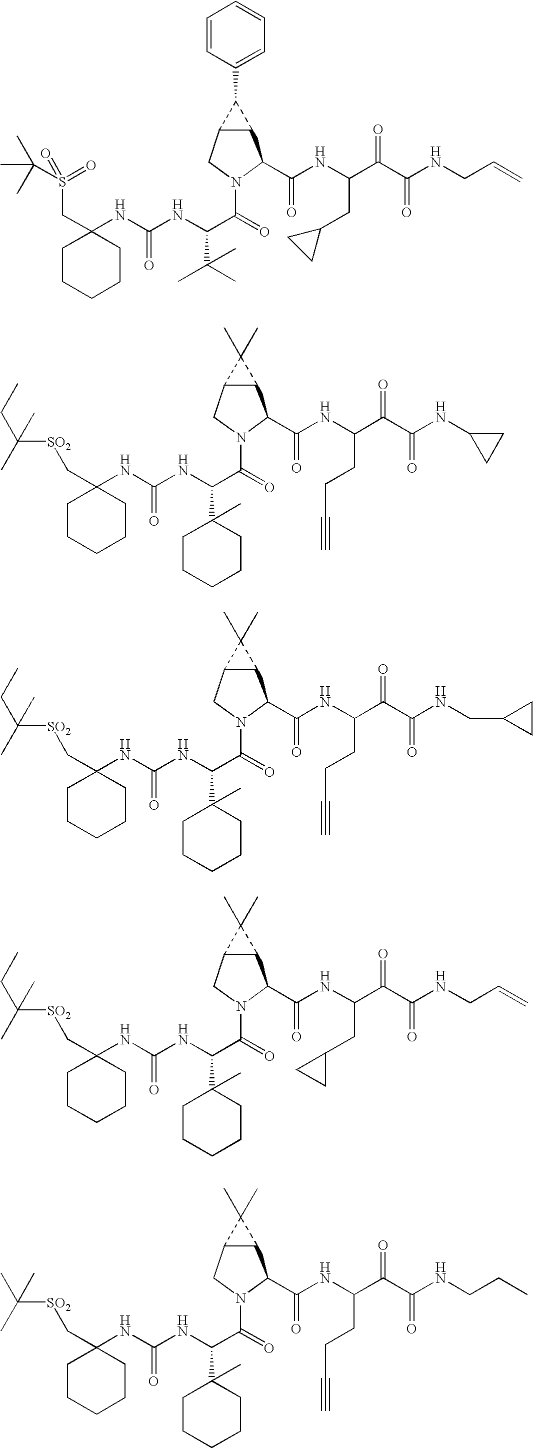 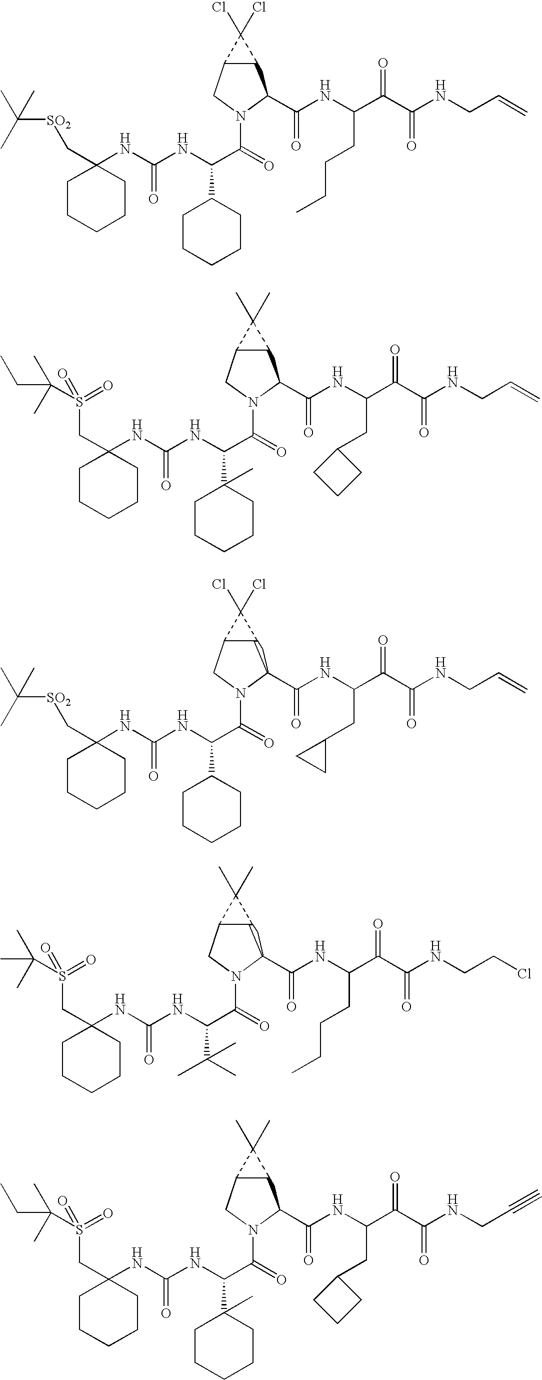 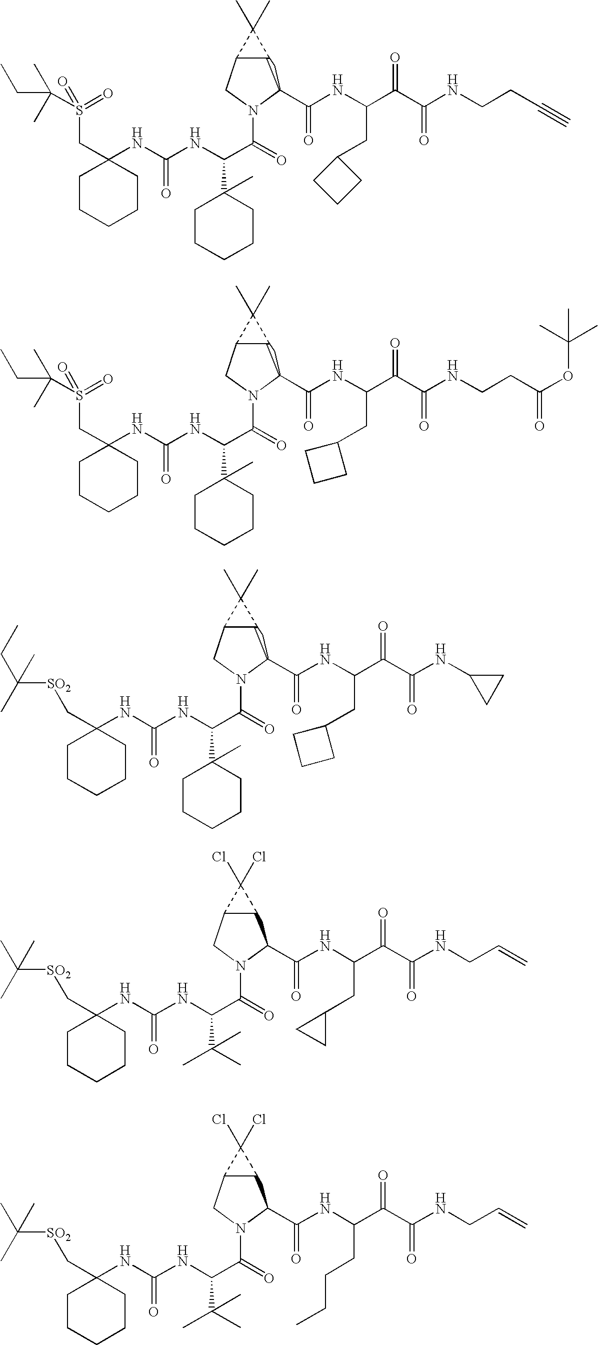 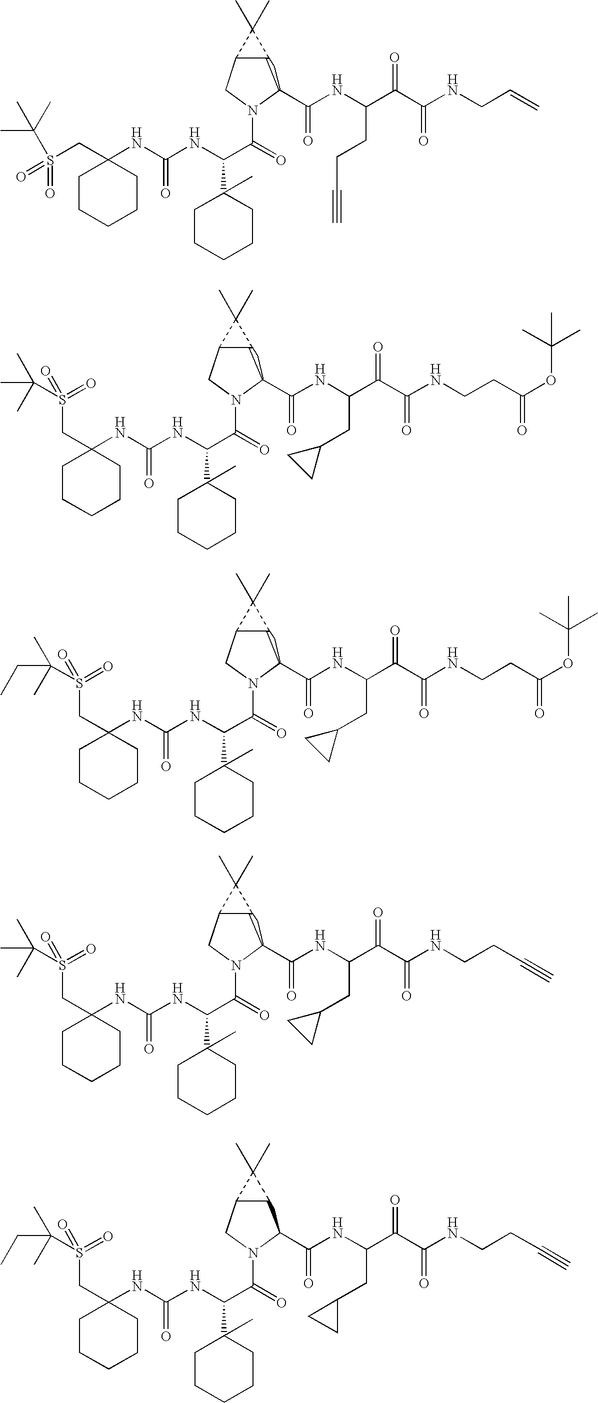 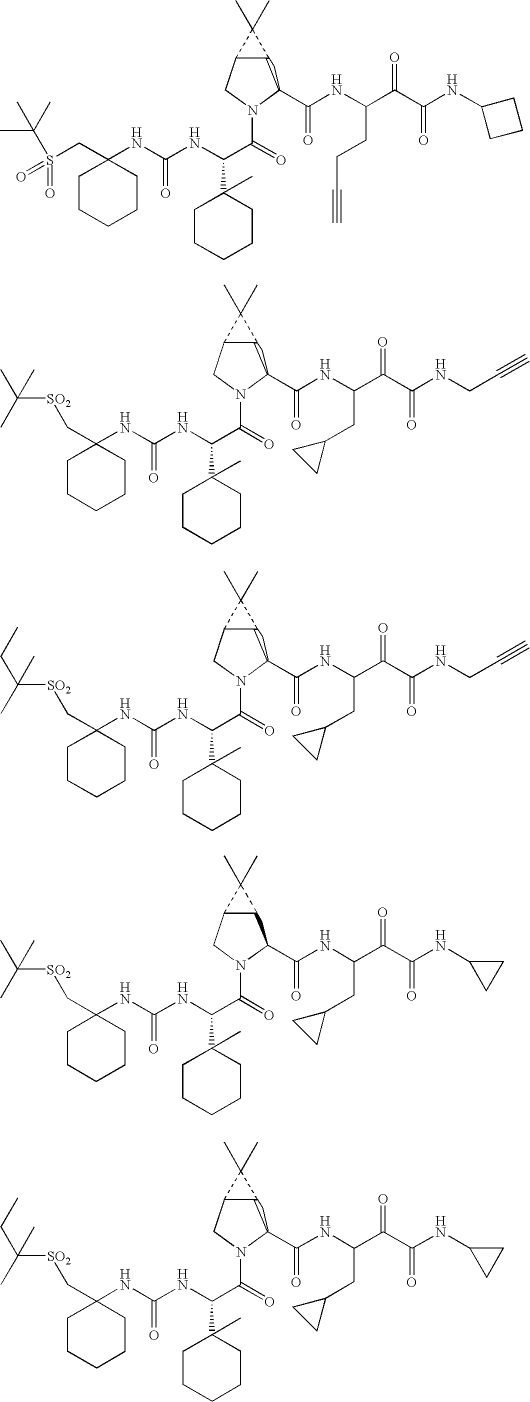 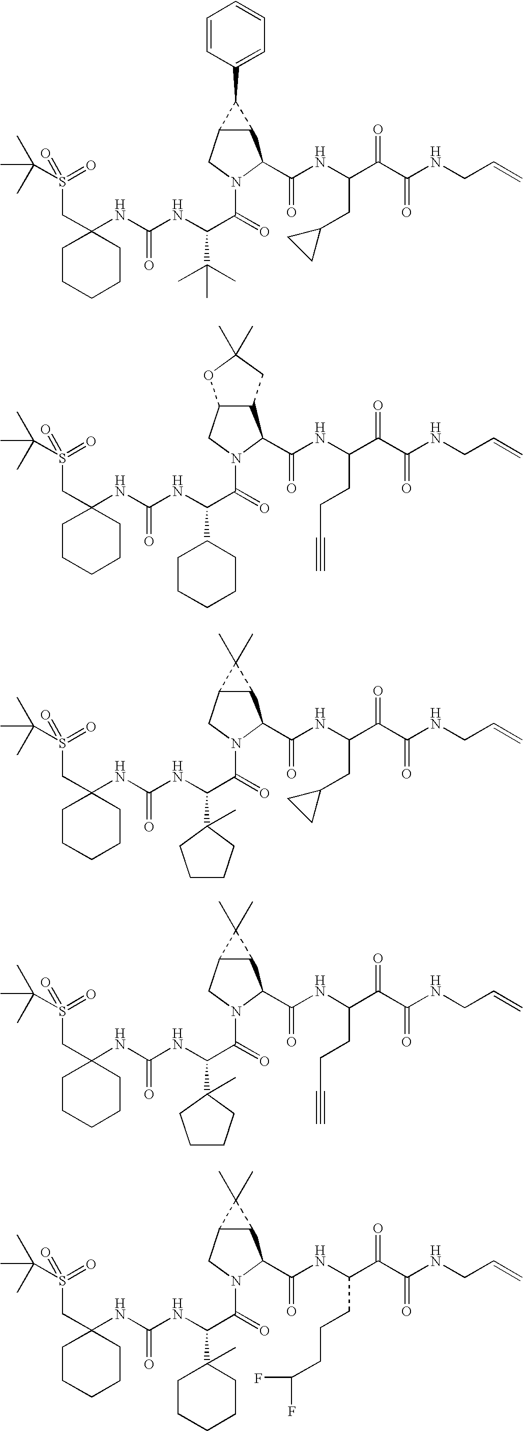 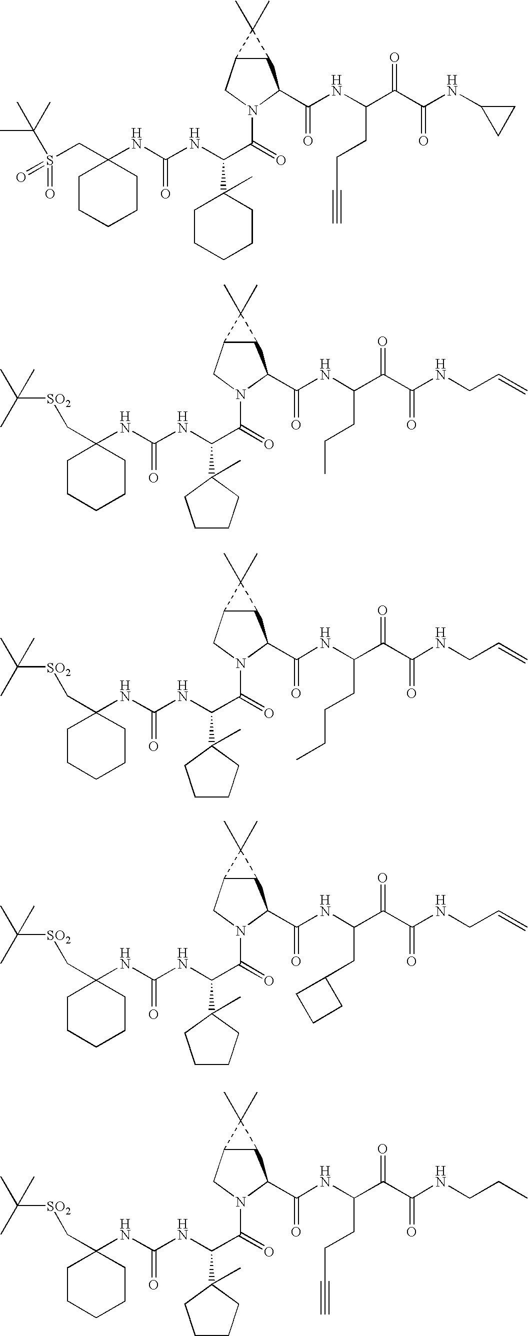 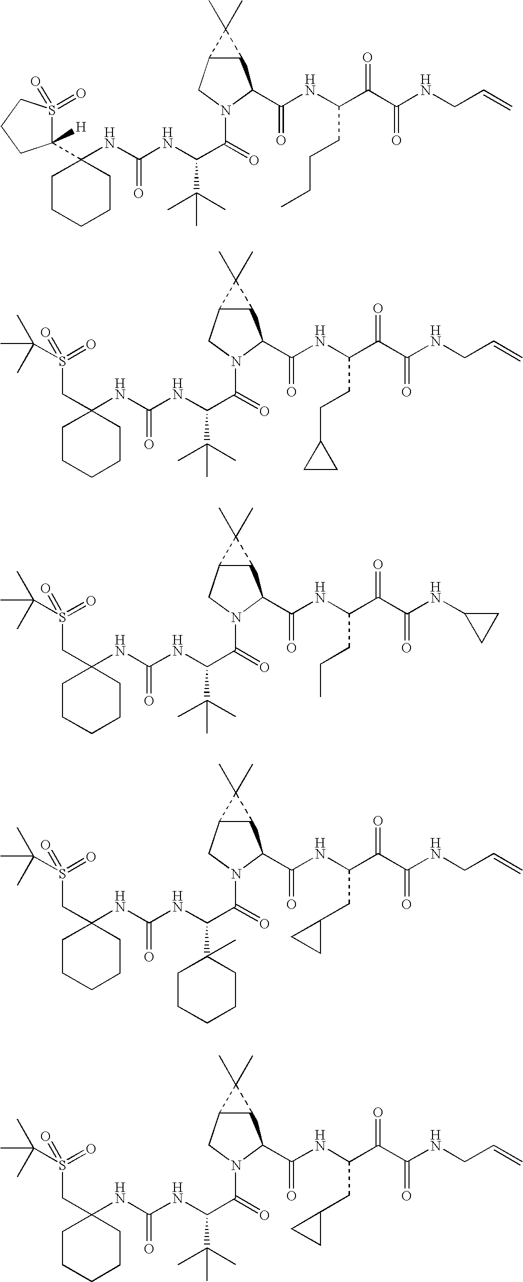 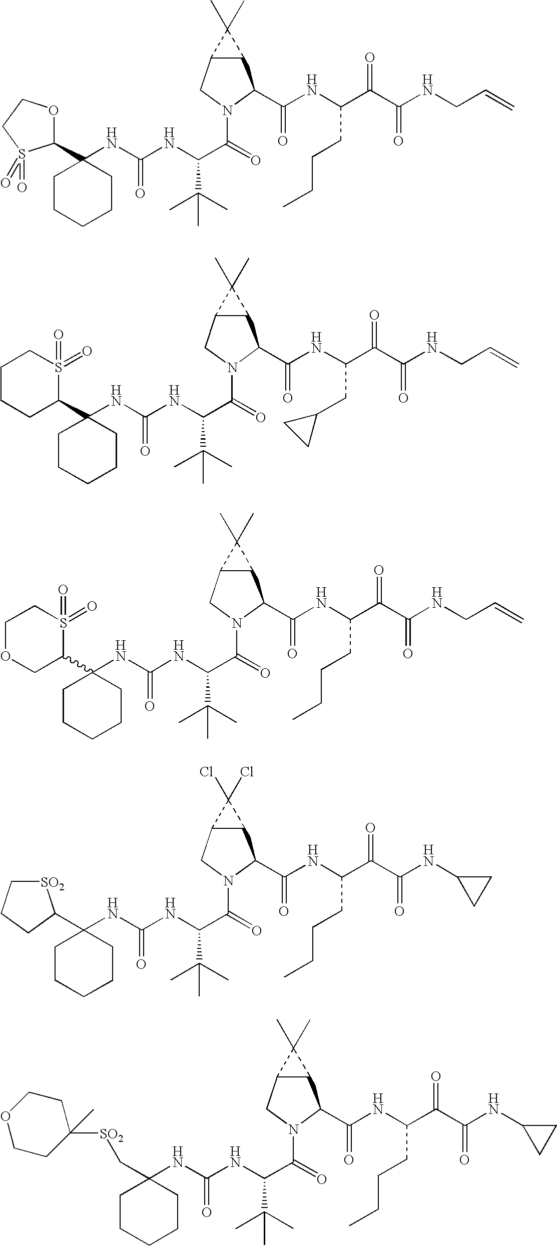 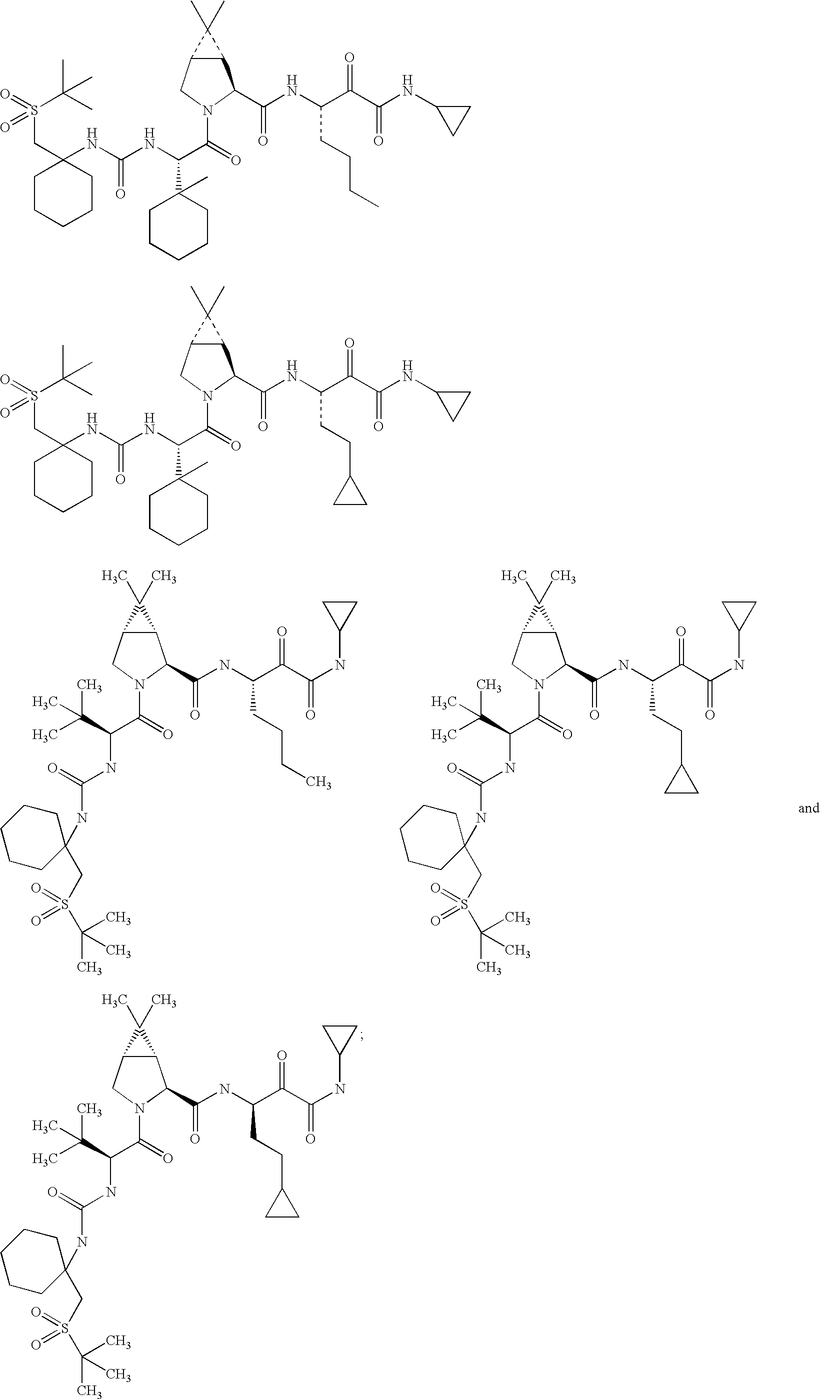
pharmaceutically acceptable salt, solvate or ester thereof.

Compounds of formula XV are disclosed in U.S. Patent Application Publication US 2005/0153900, published Jul. 14, 2005, and the corresponding PCT publication WO2005/058821, published Jun. 30, 2005, both of which are incorporated herein by reference.

Non-limiting examples of certain compounds disclosed in these publications are: 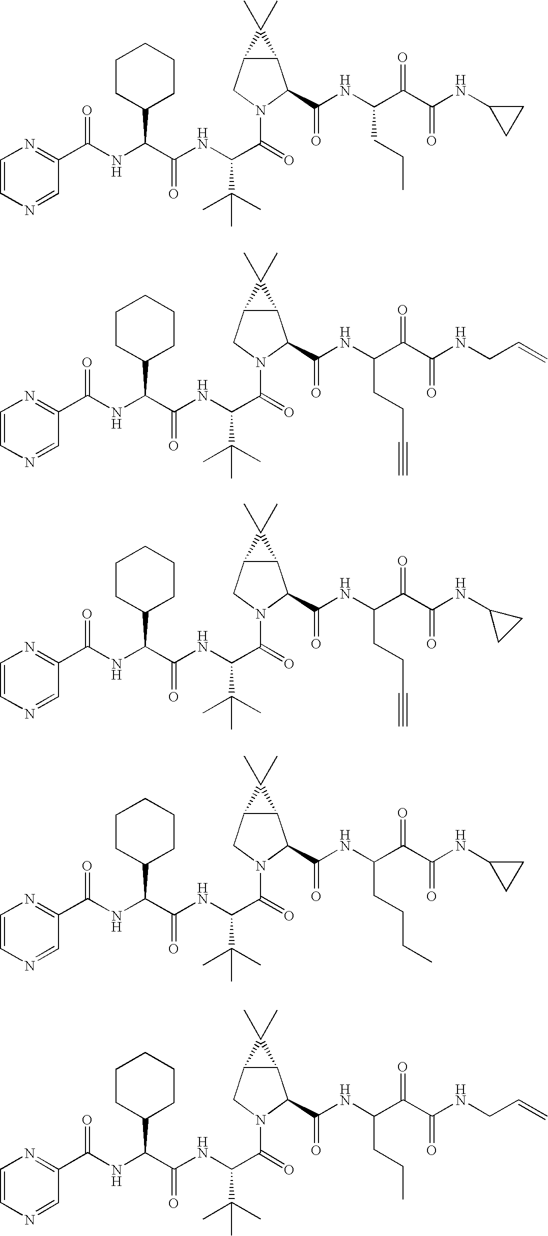 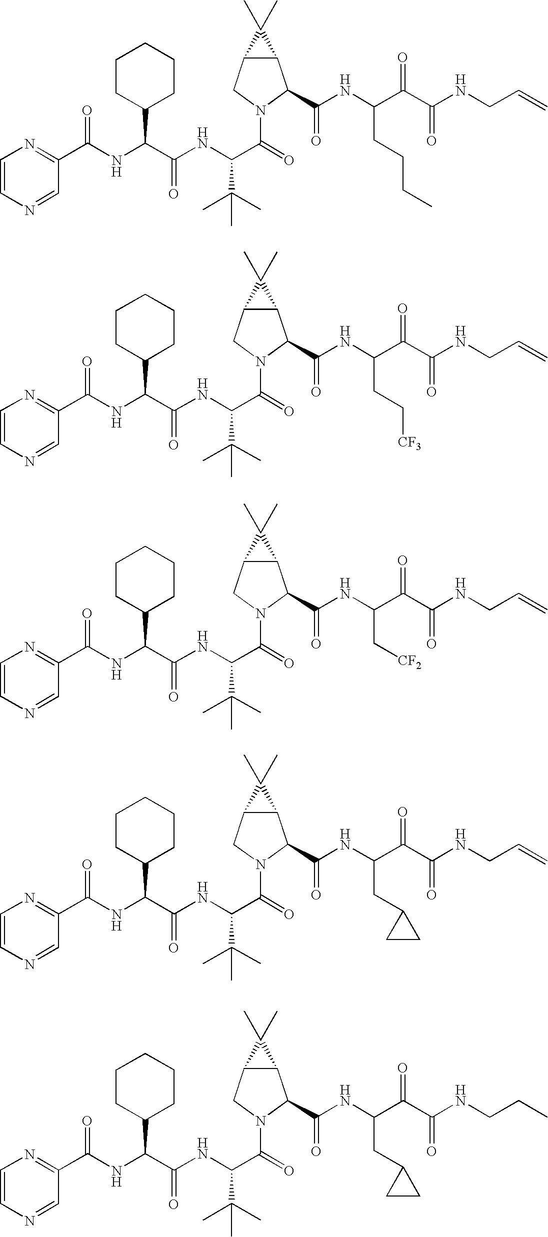 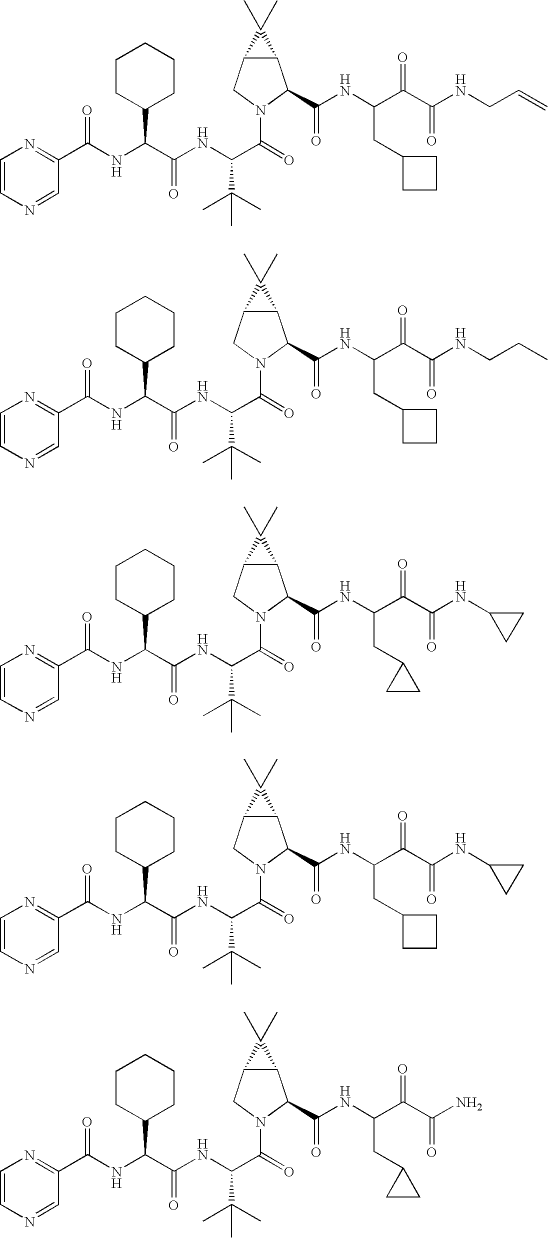 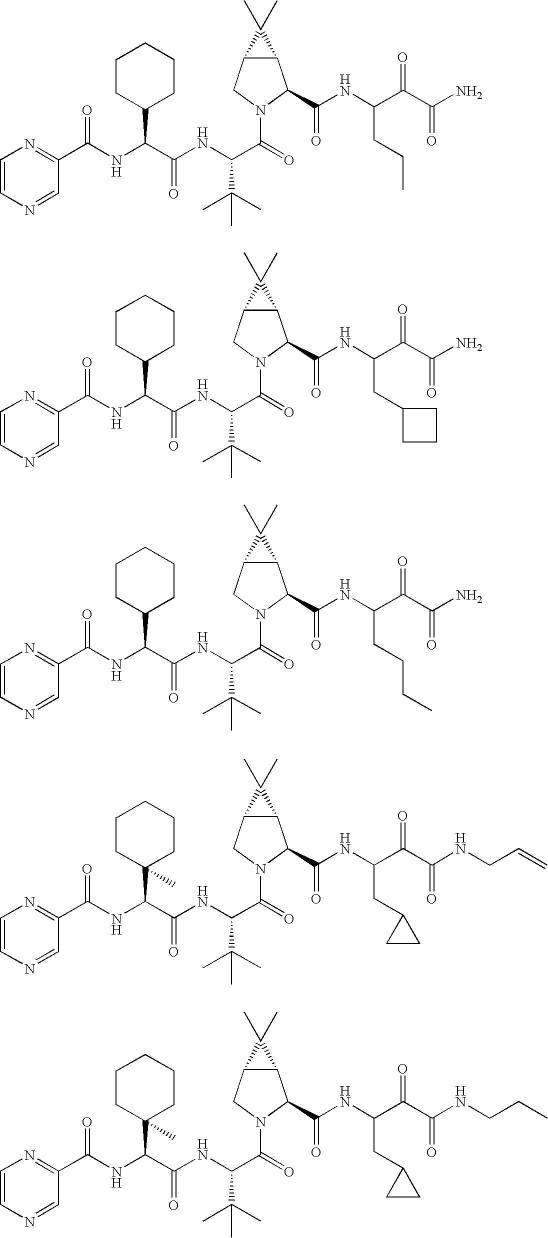 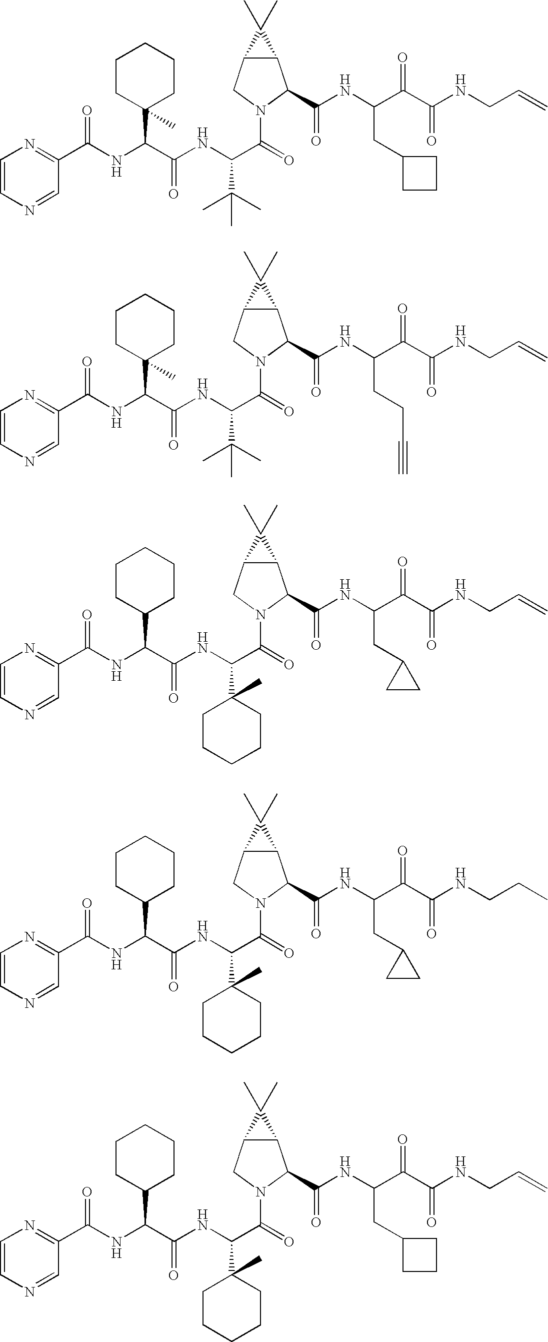 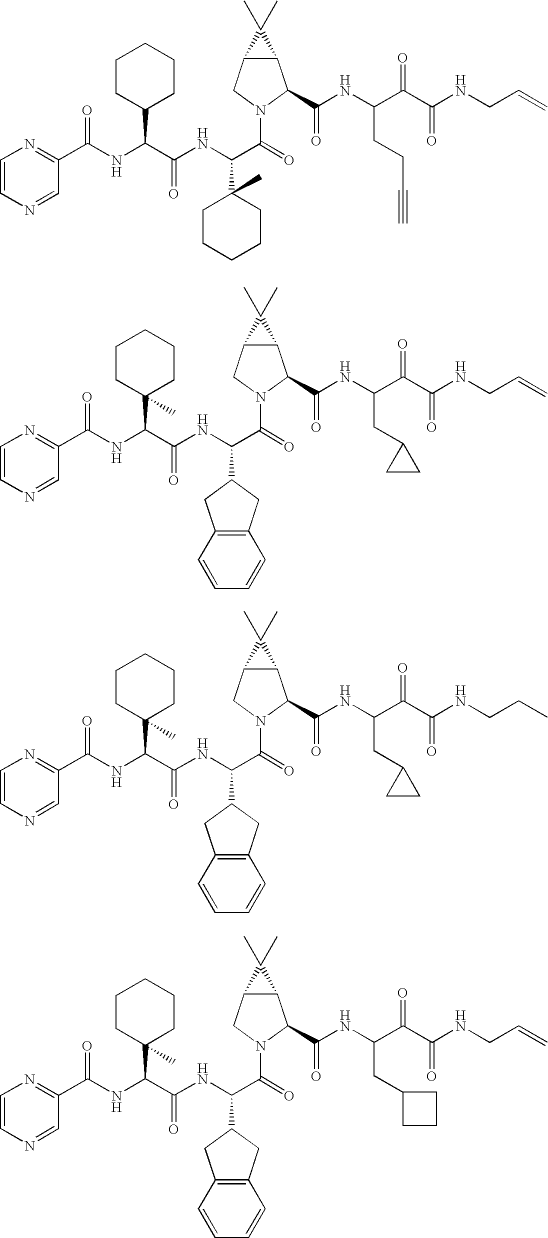 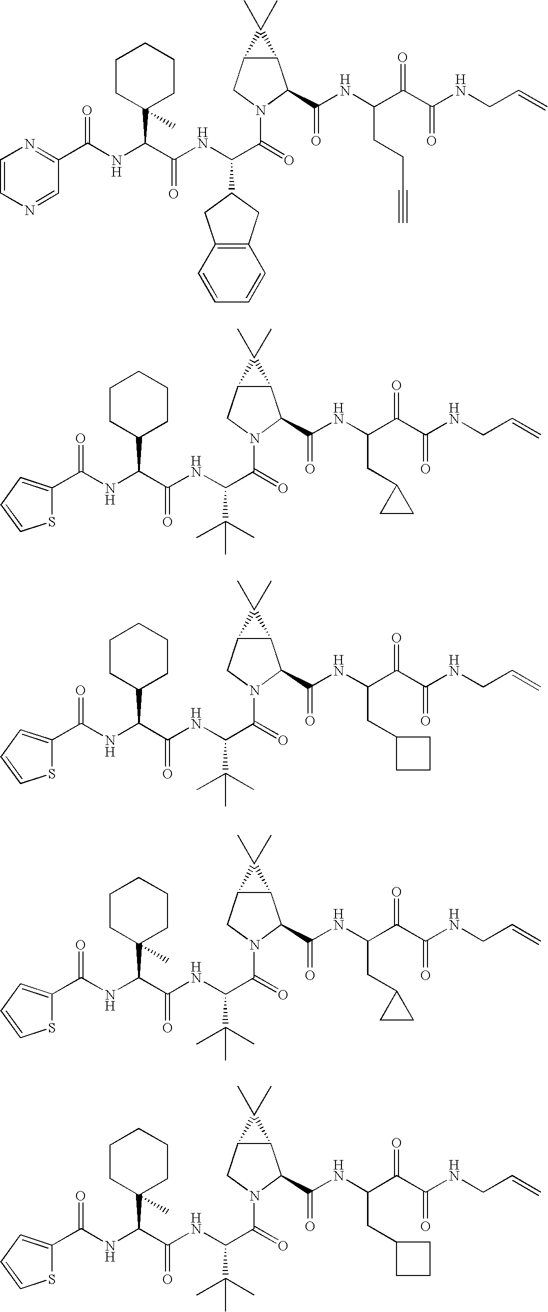 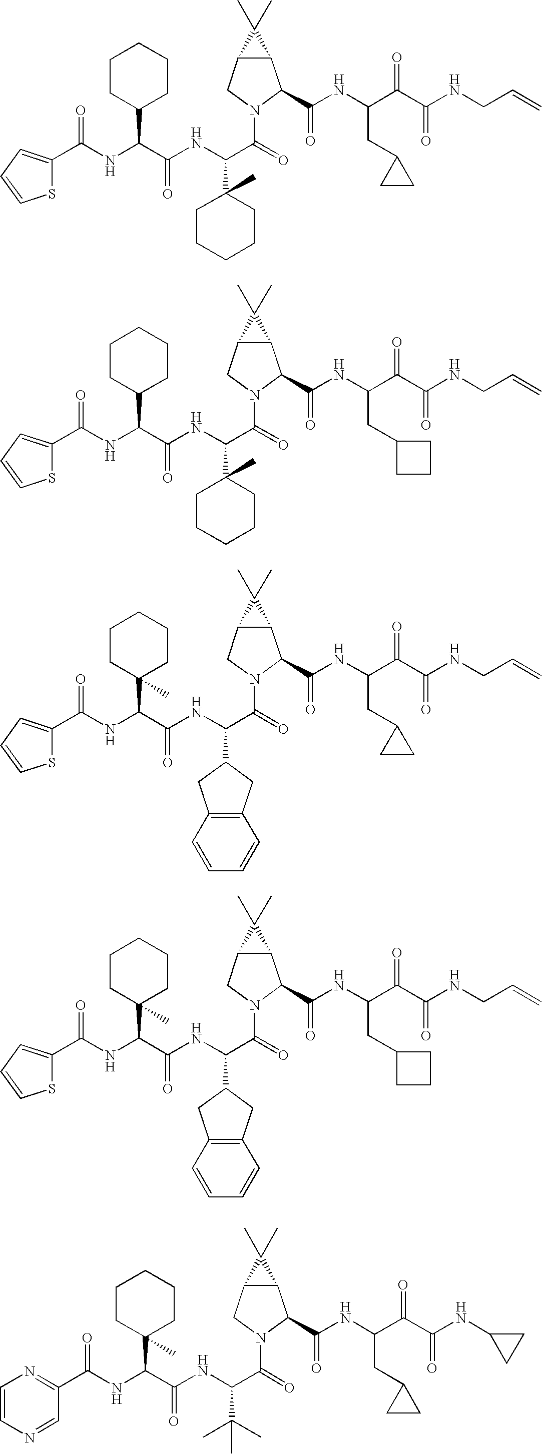 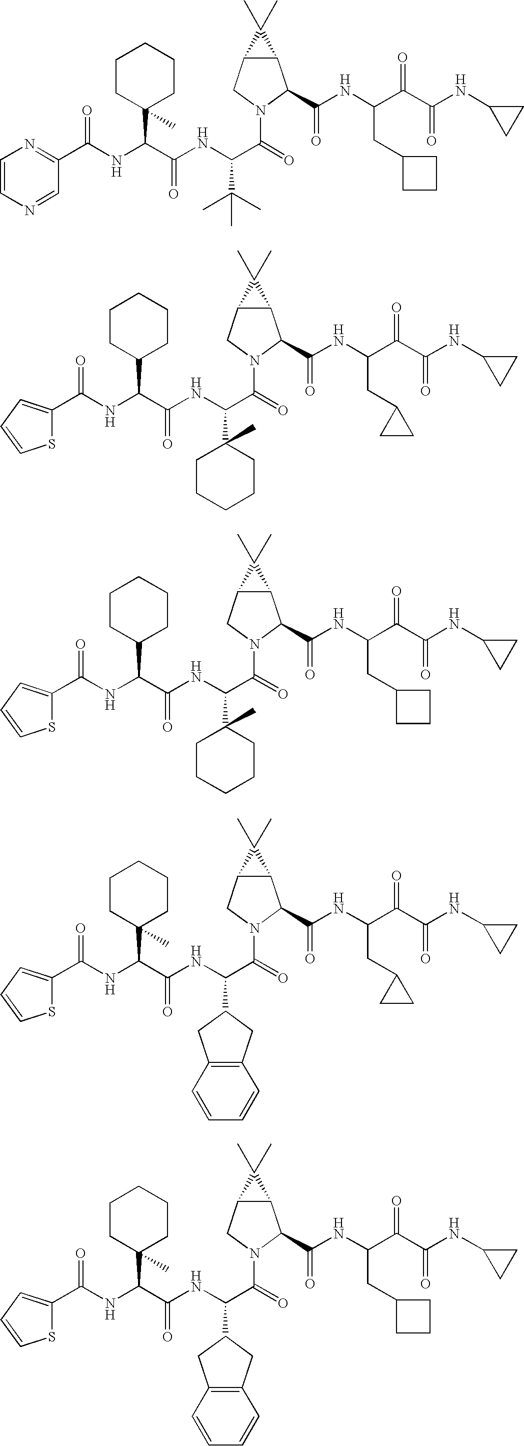 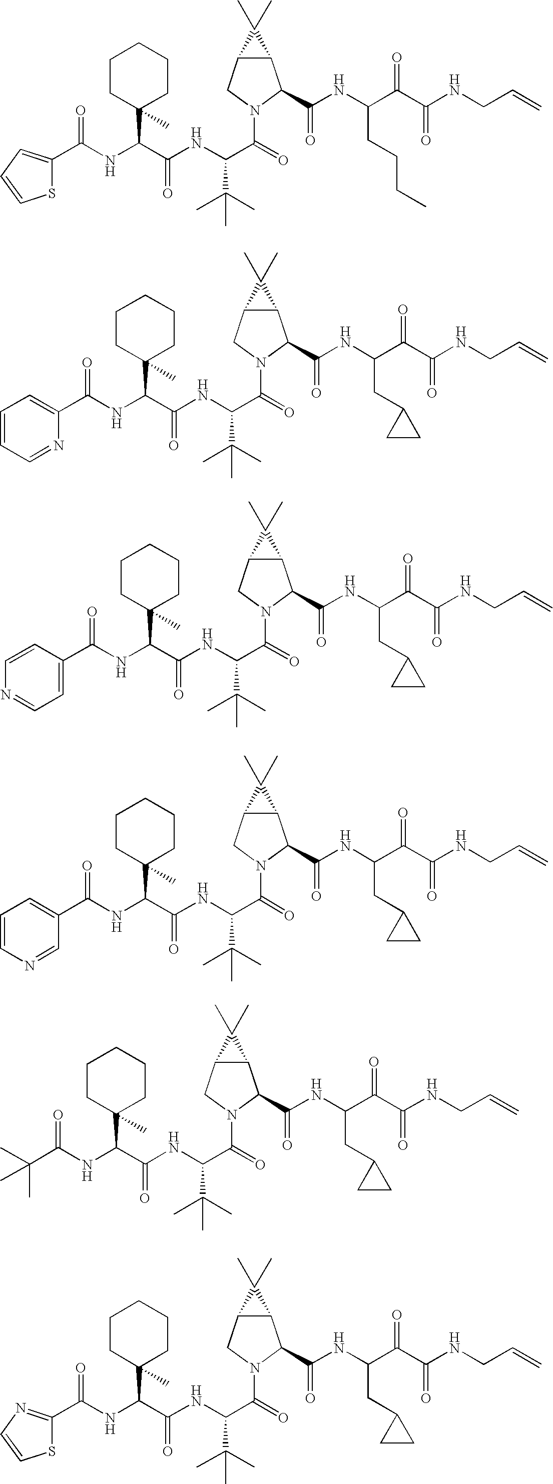 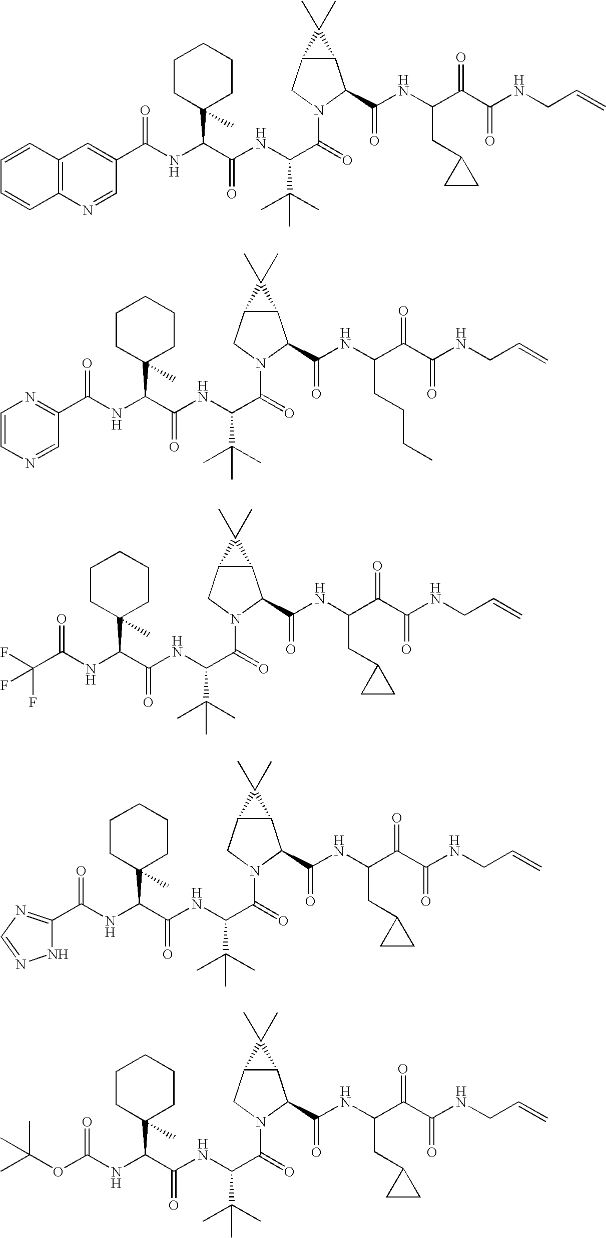 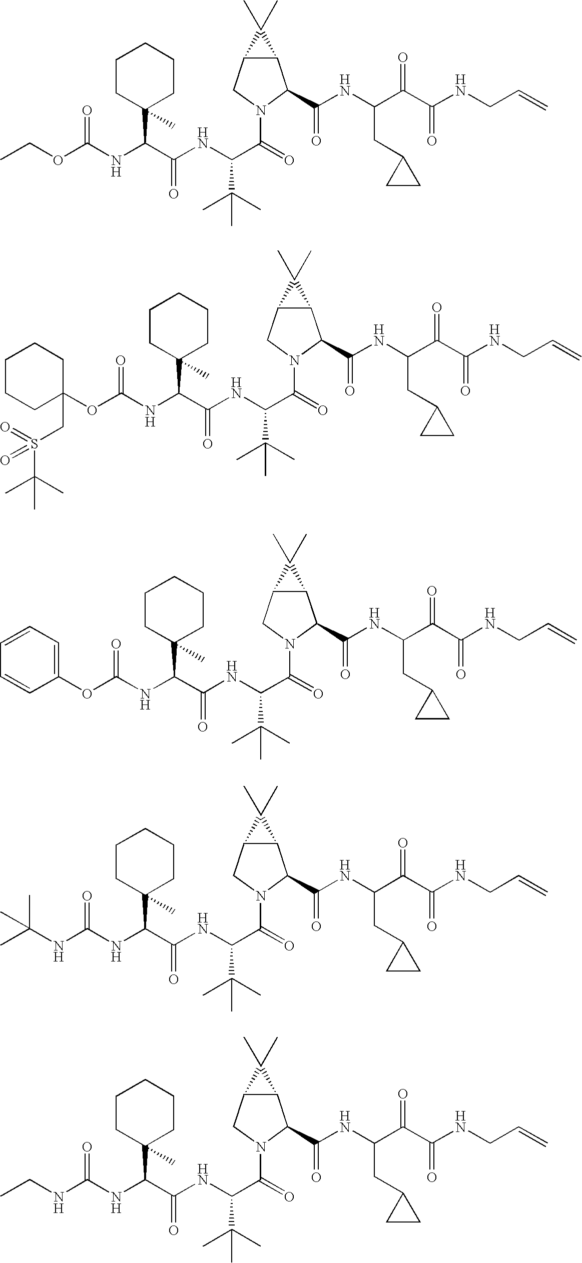 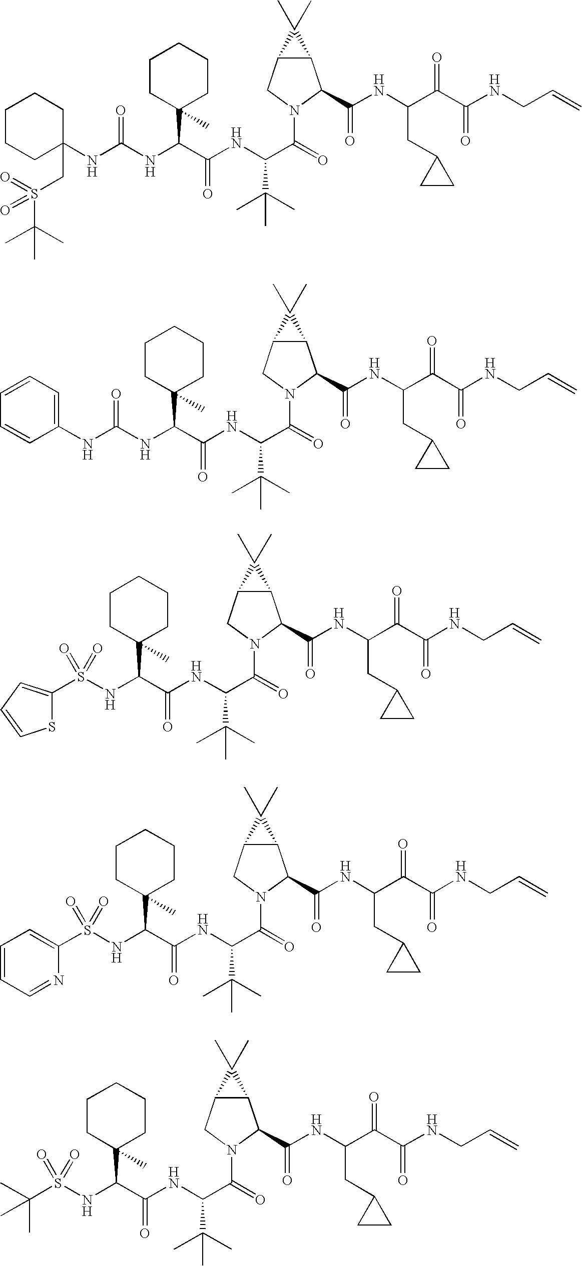 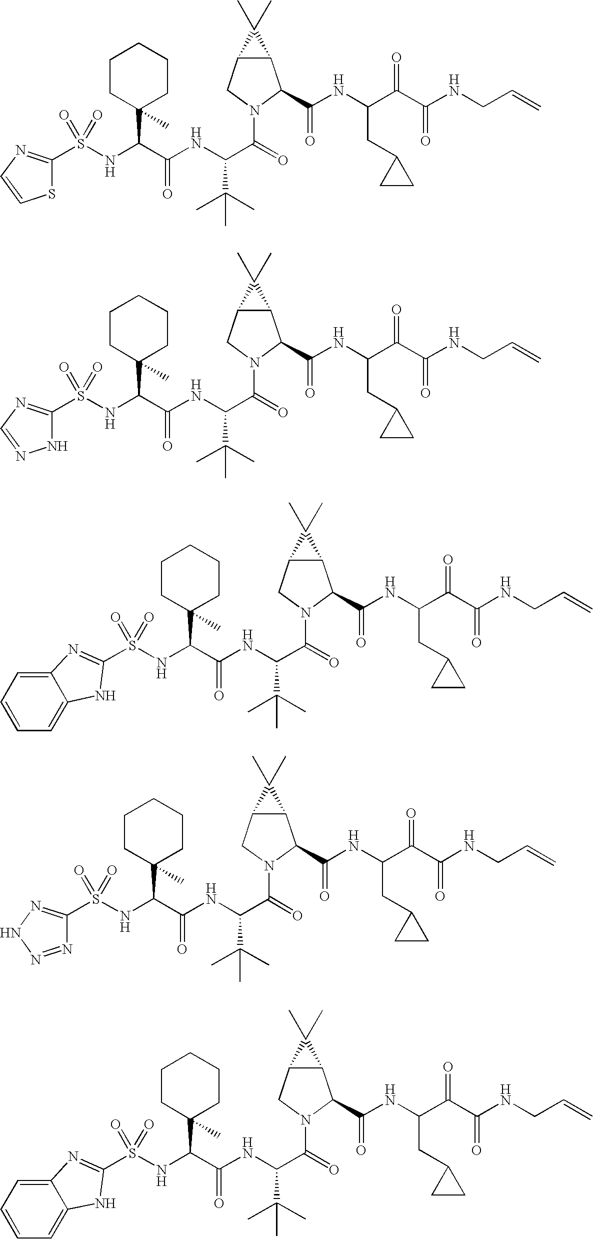 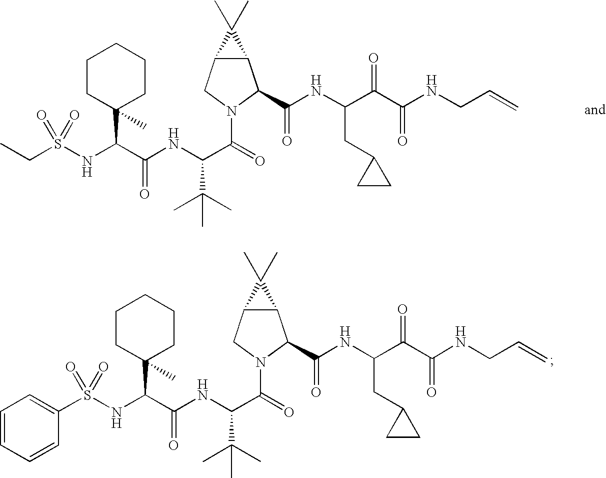
or a pharmaceutically acceptable salt, solvate or ester thereof.

Compounds of formula XVI are disclosed in U.S. Patent Application Publication 2005/0197301, published on Sep. 8, 2005, and the corresponding PCT publication WO2005/1087730, published on Sep. 22, 2005 The preparation of these compounds is disclosed in the experimental section of this application set forth hereinbelow.

Non-limiting examples of certain compounds disclosed in these publications are: 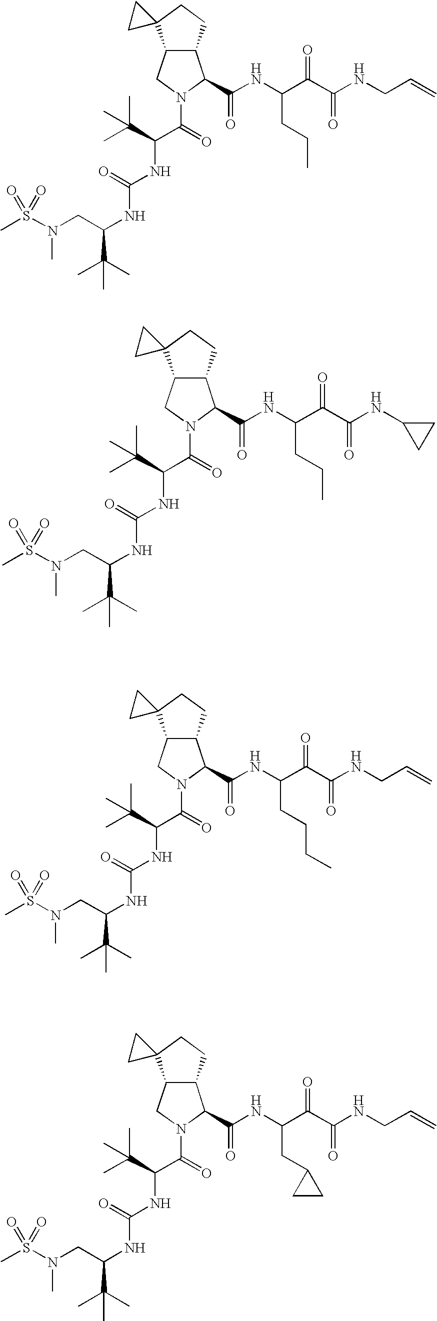 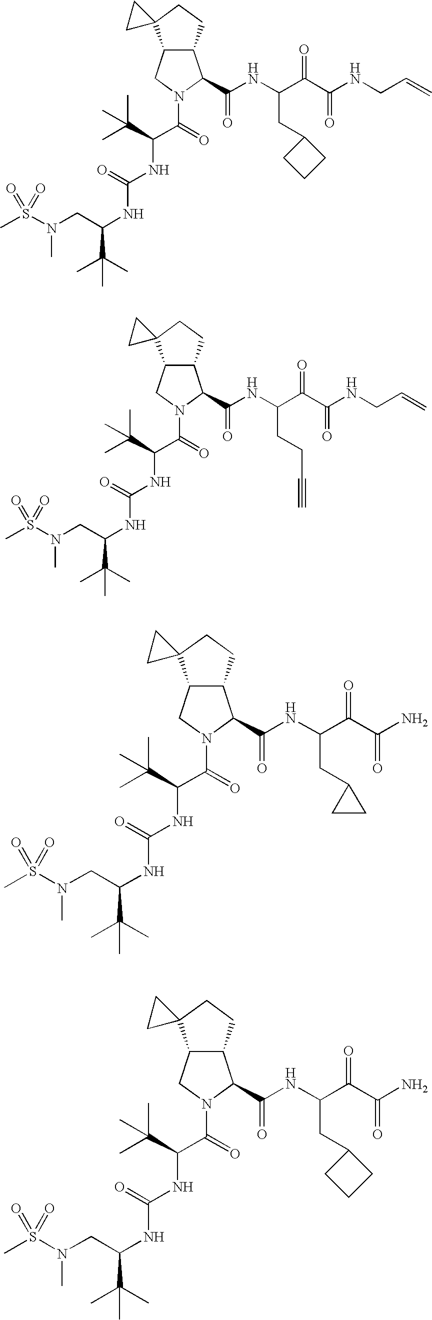 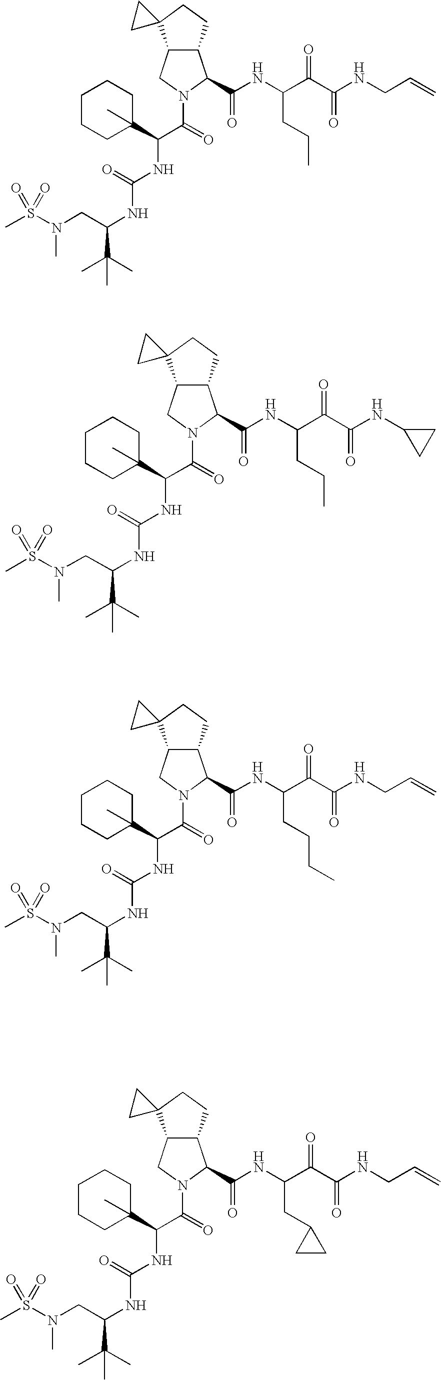 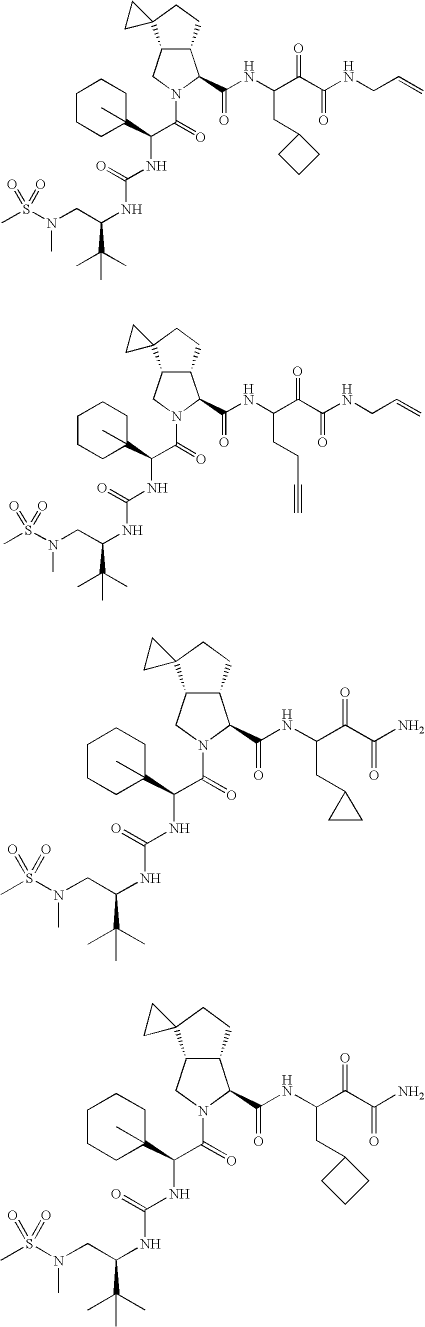 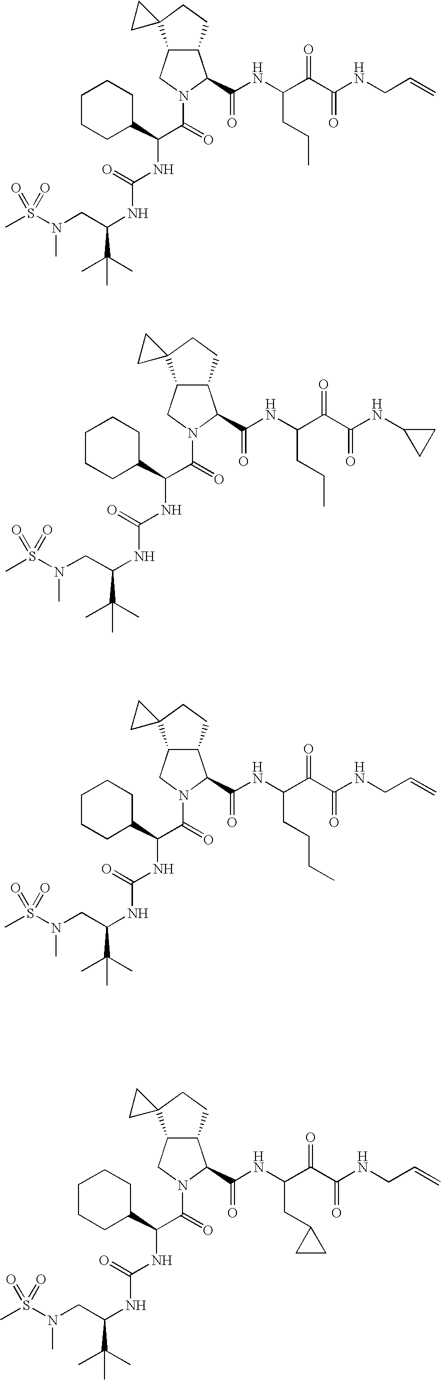 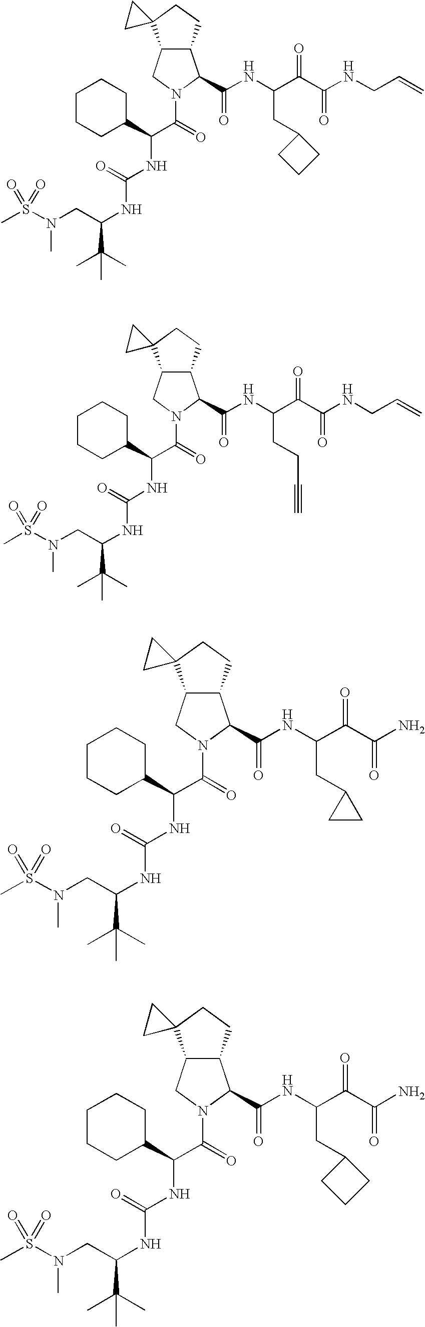 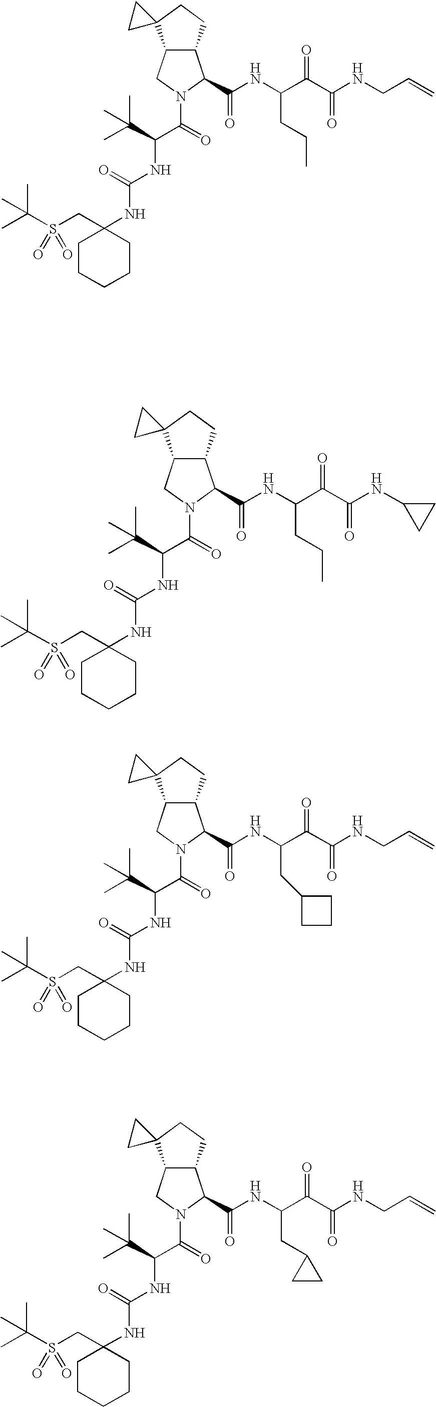 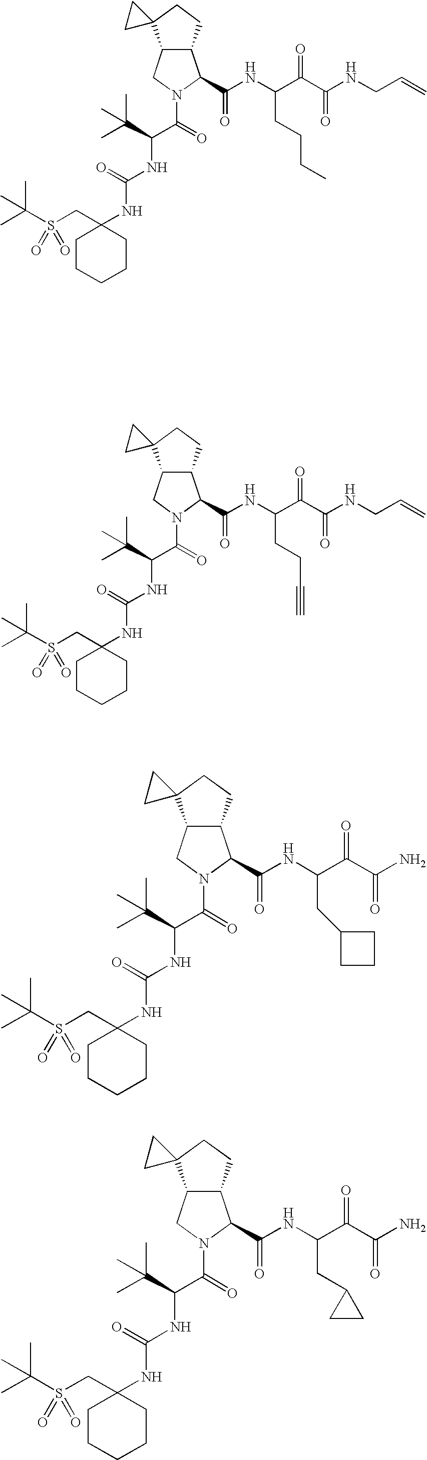 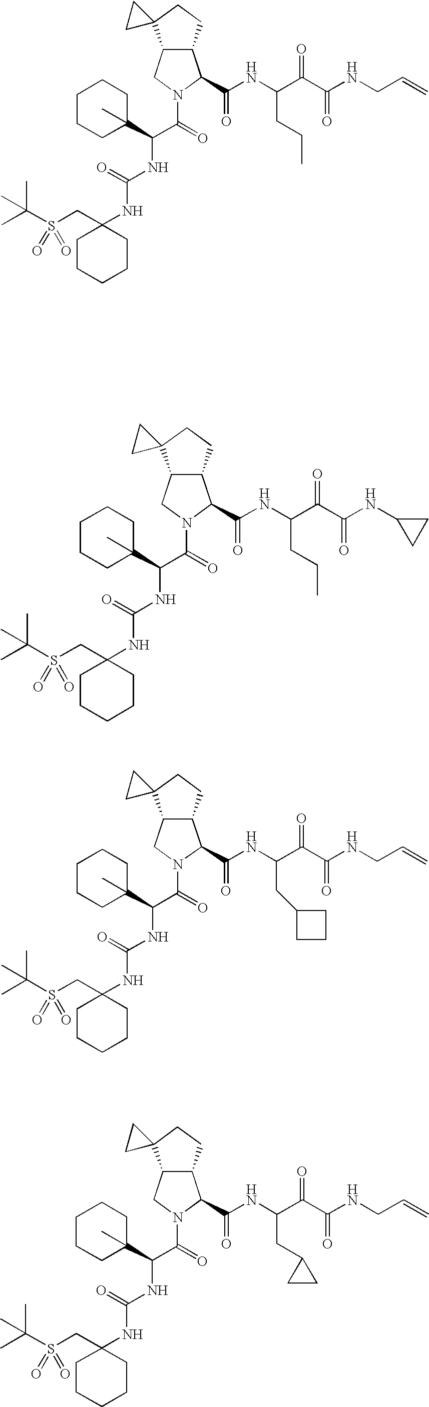 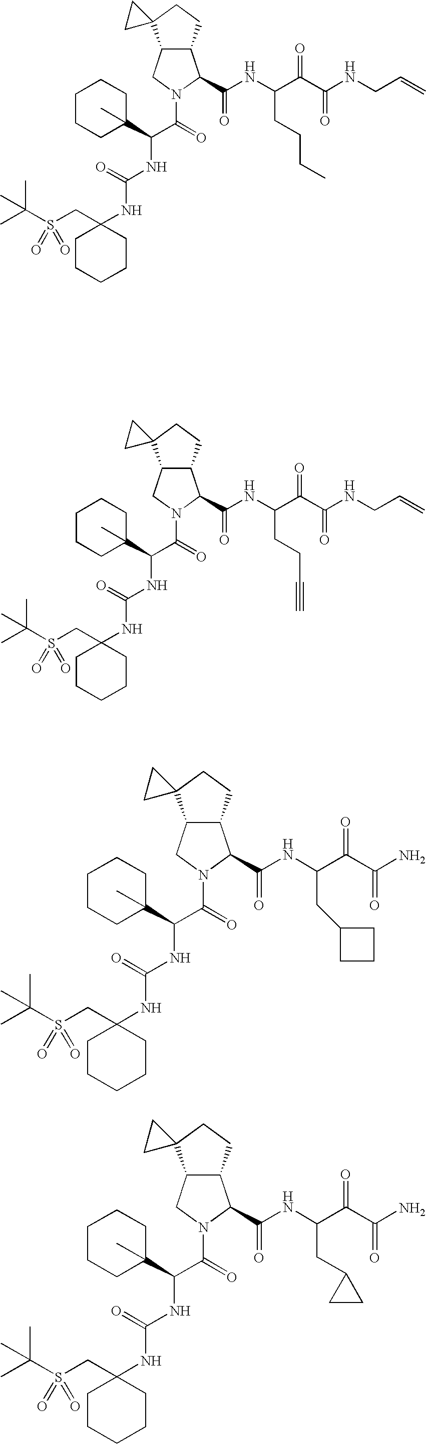 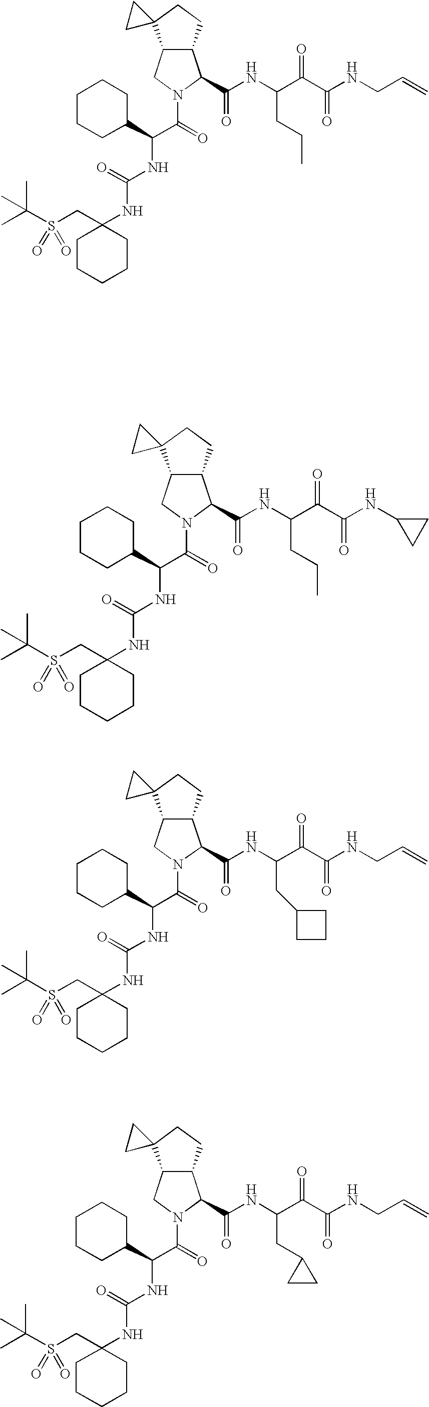 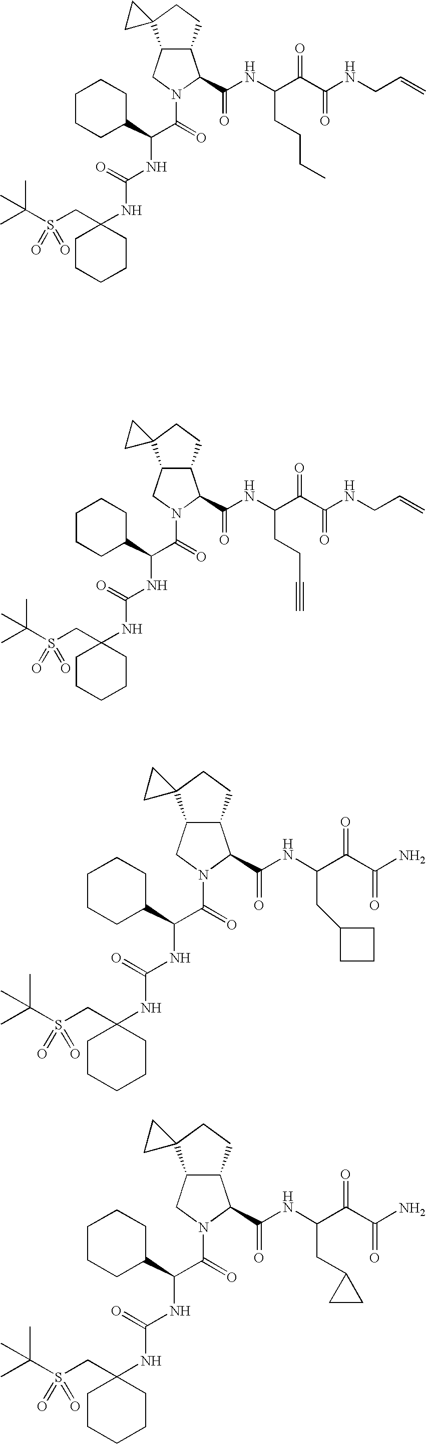 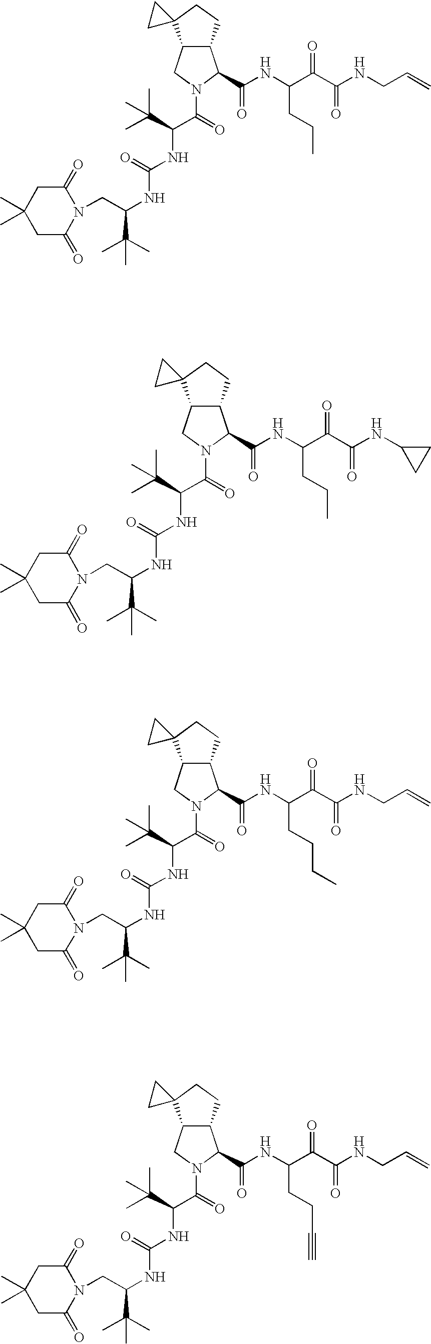 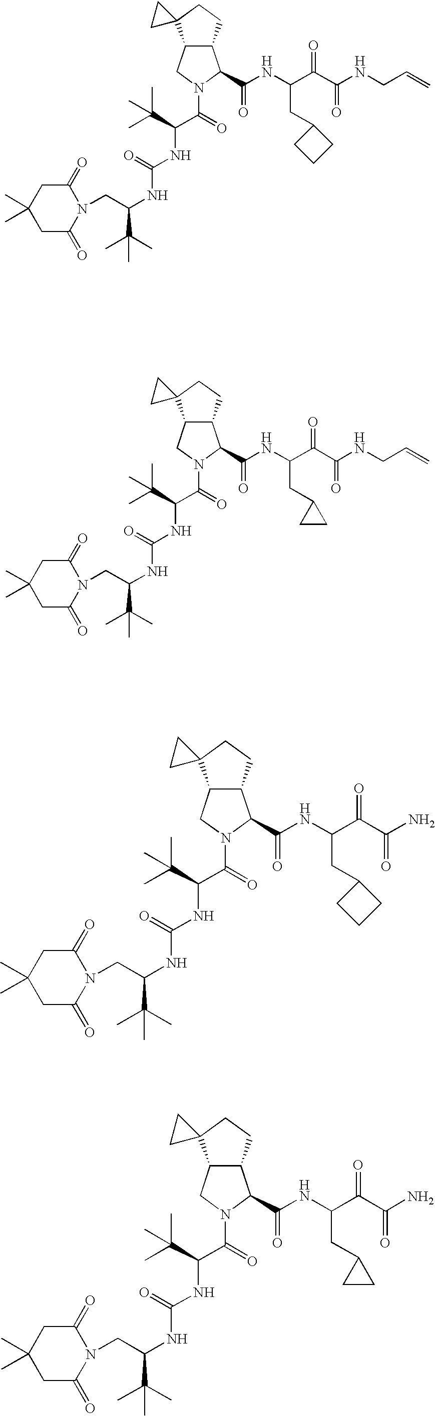 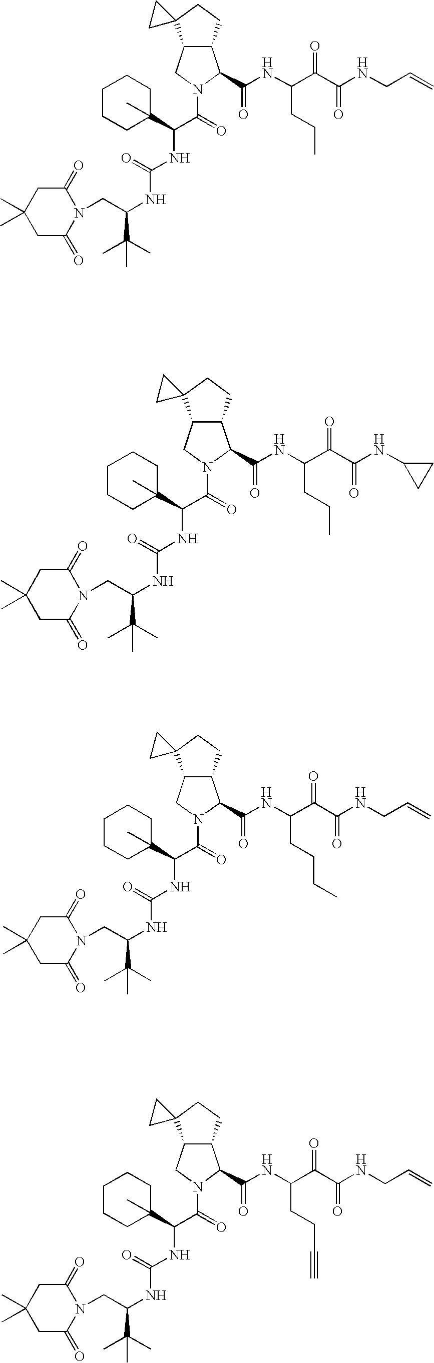 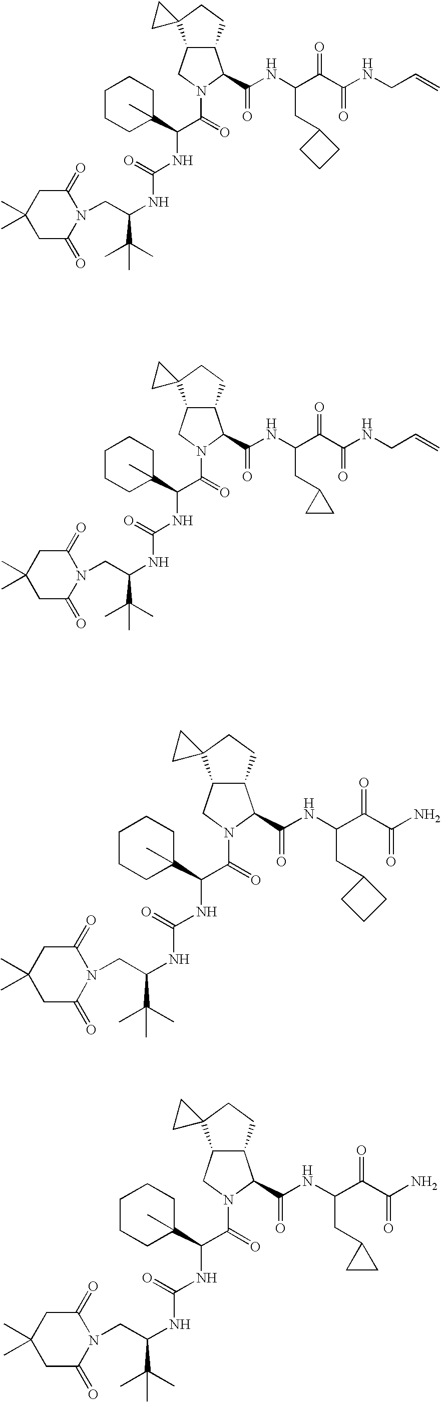 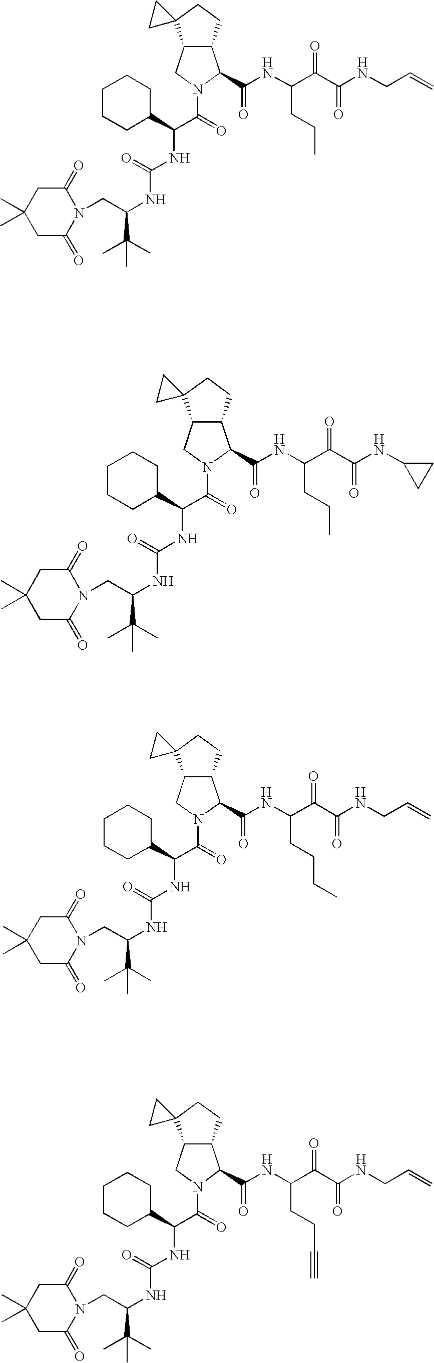 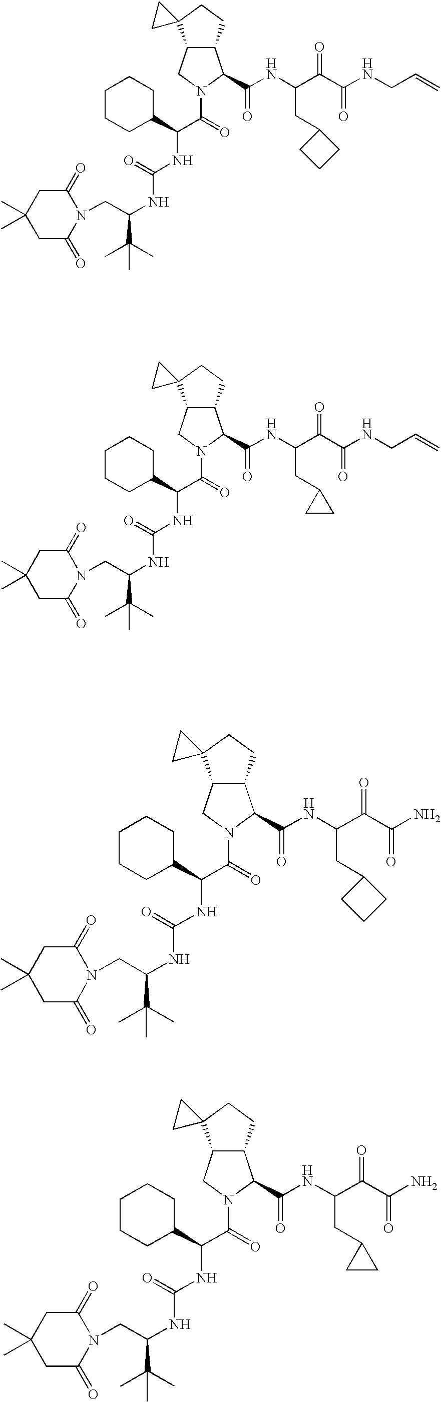 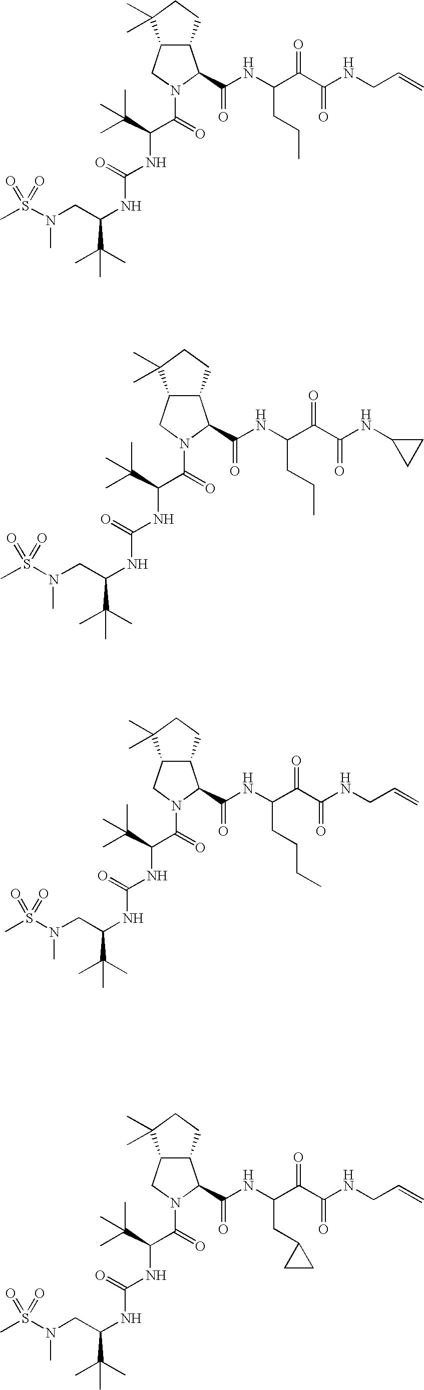 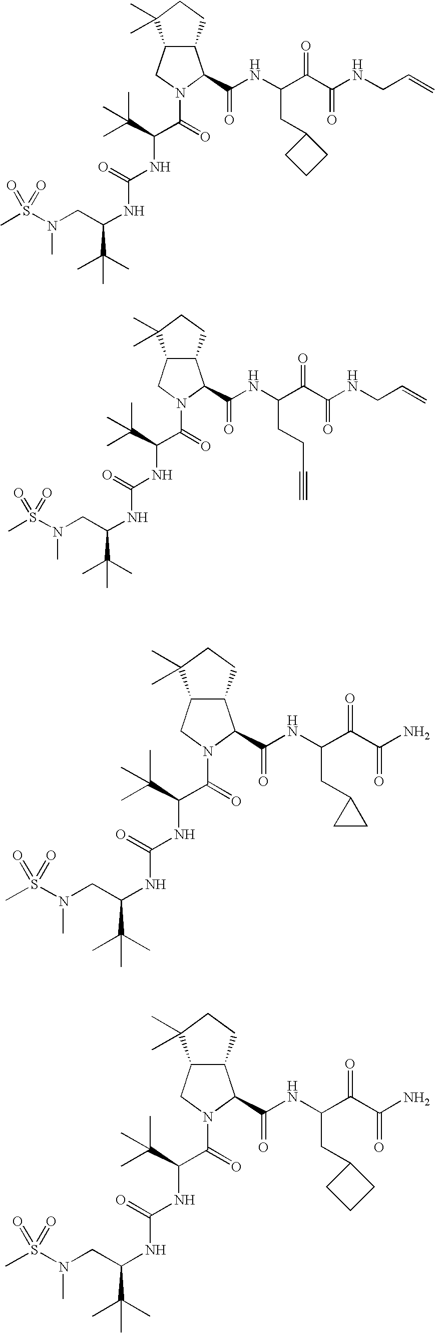 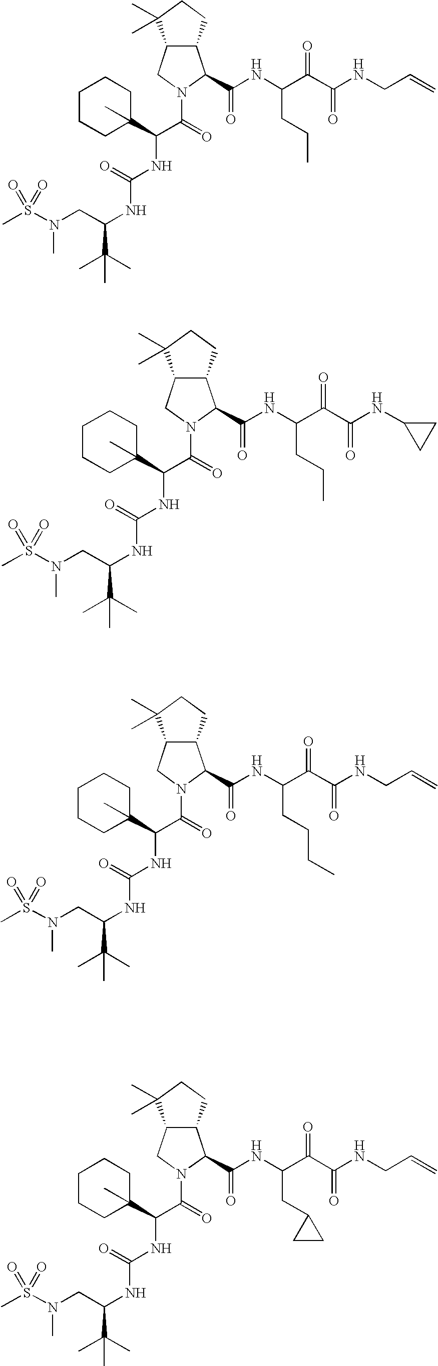 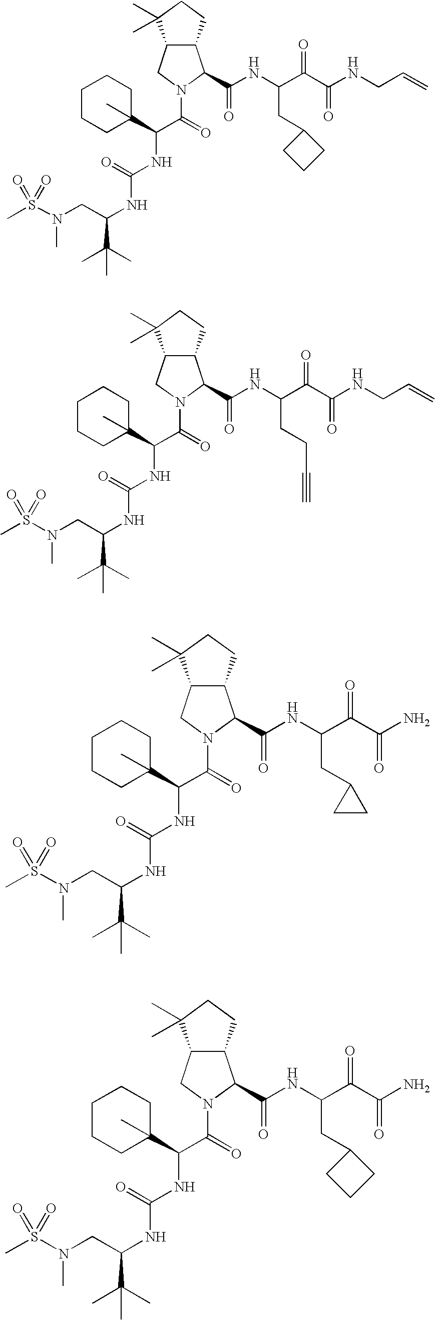 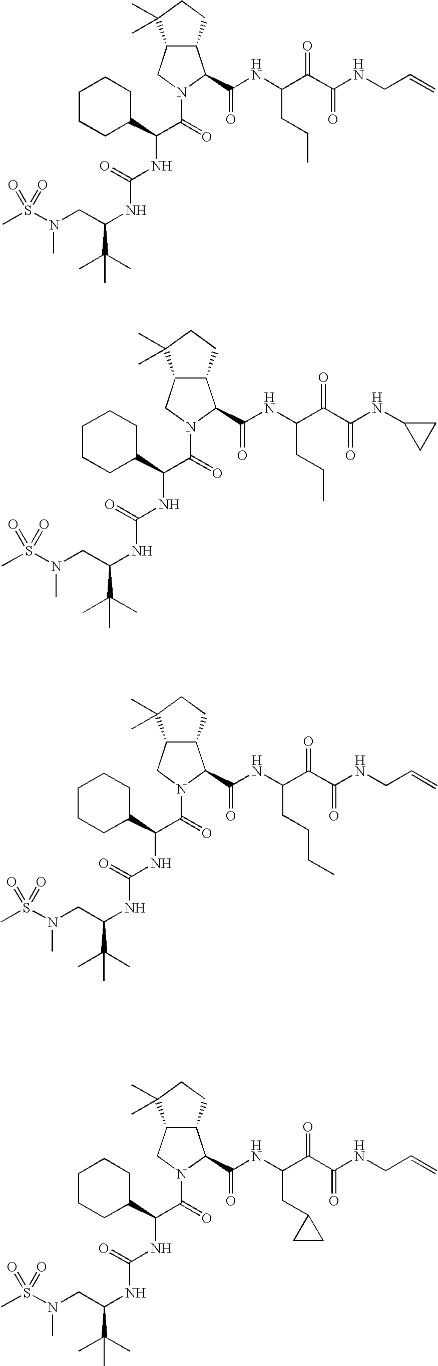 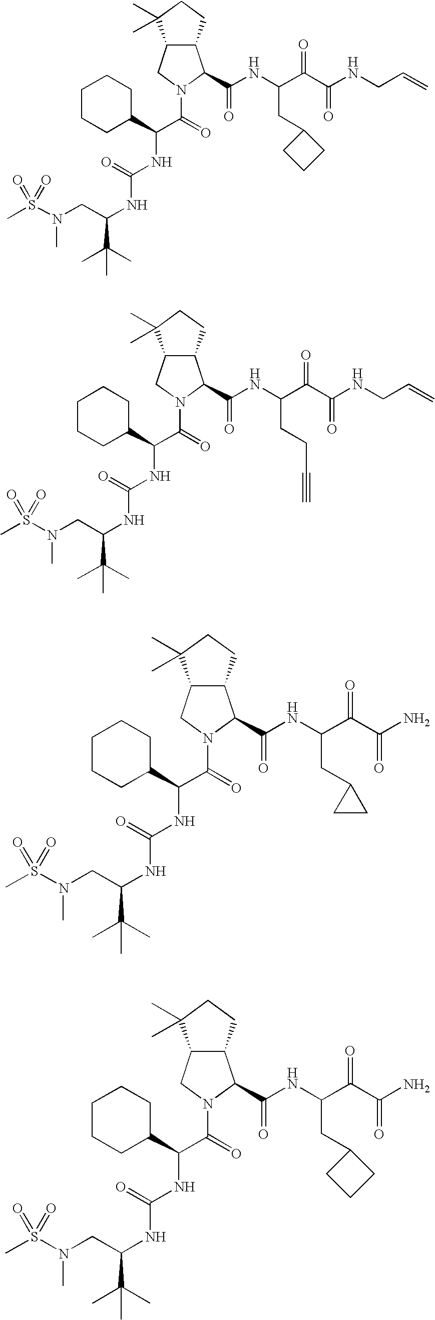 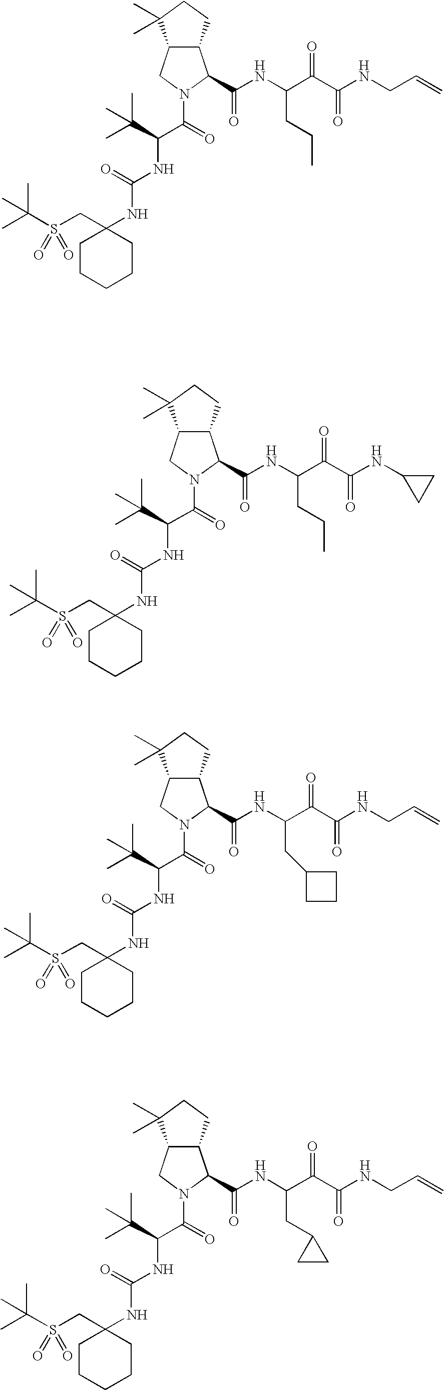 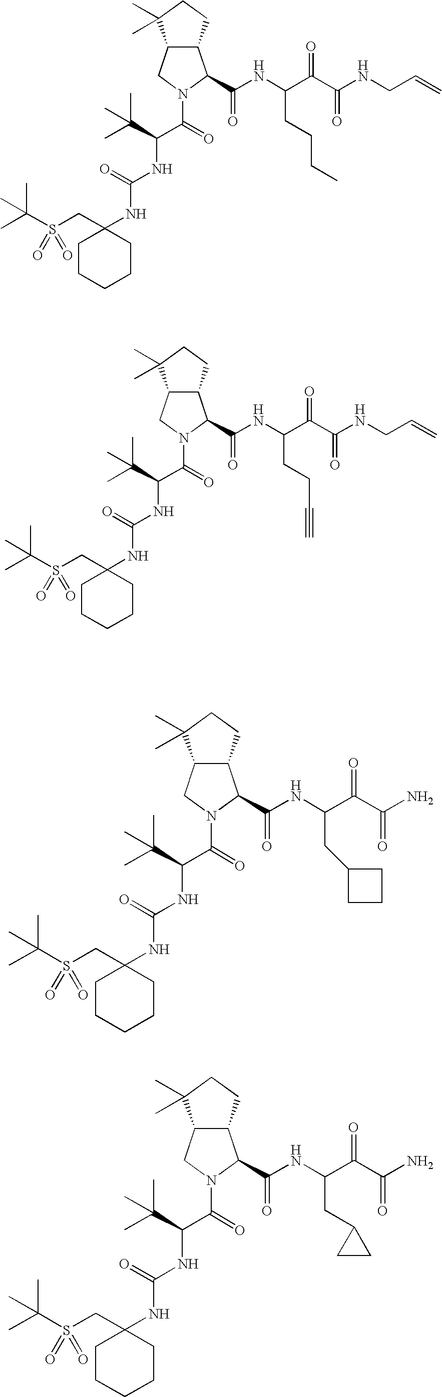 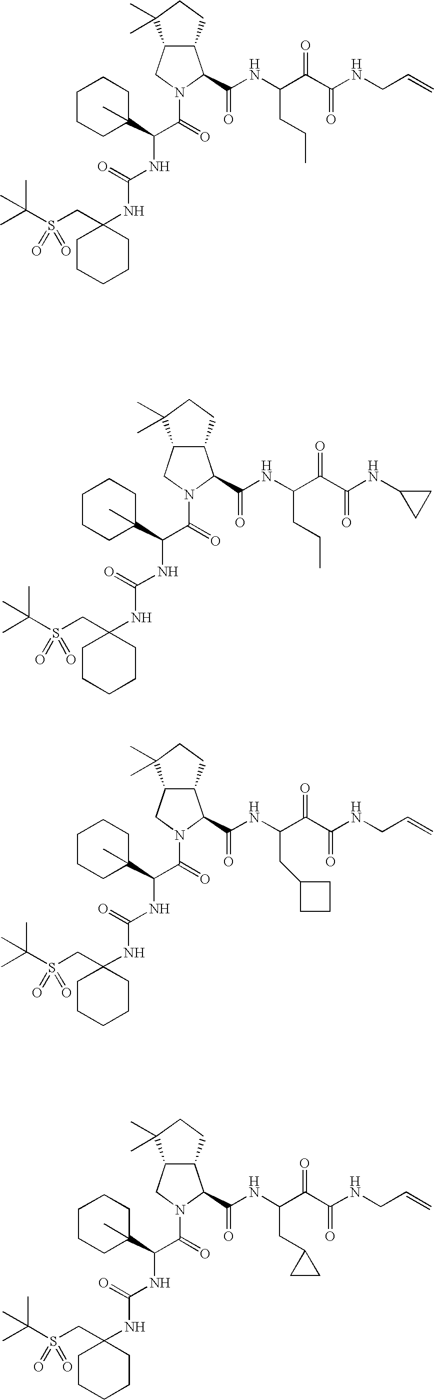 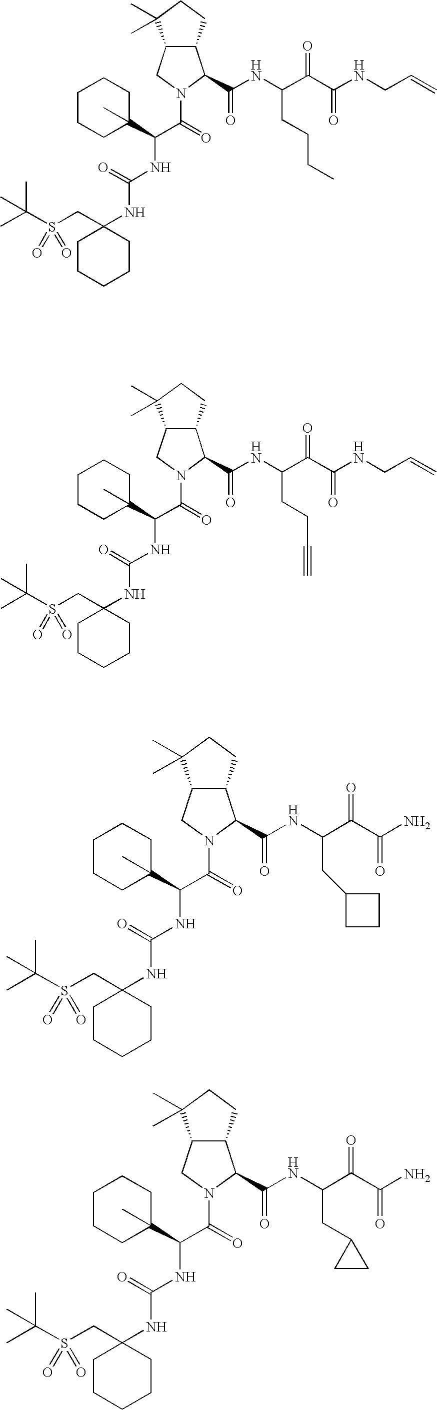 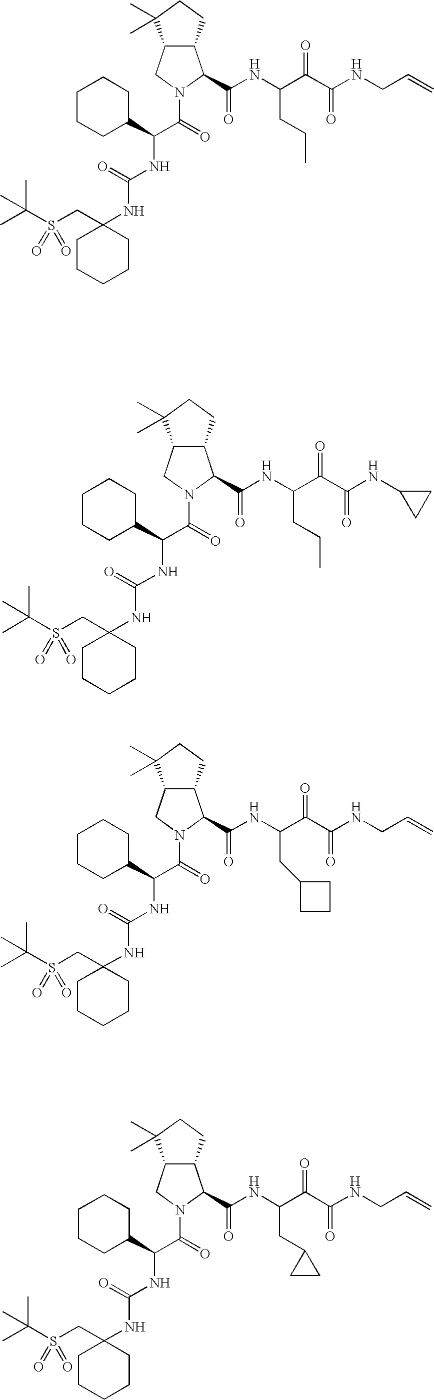 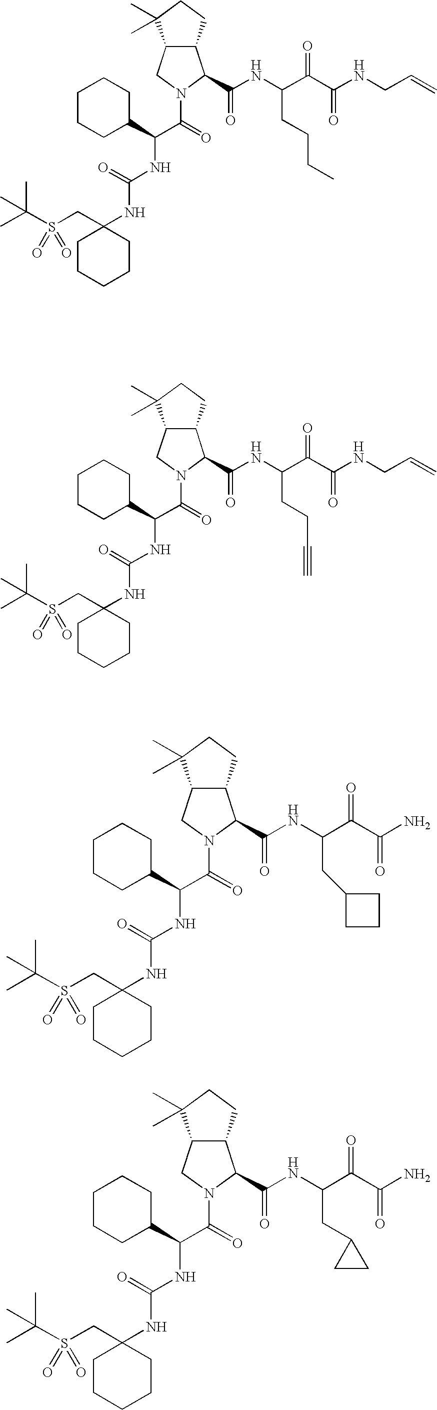 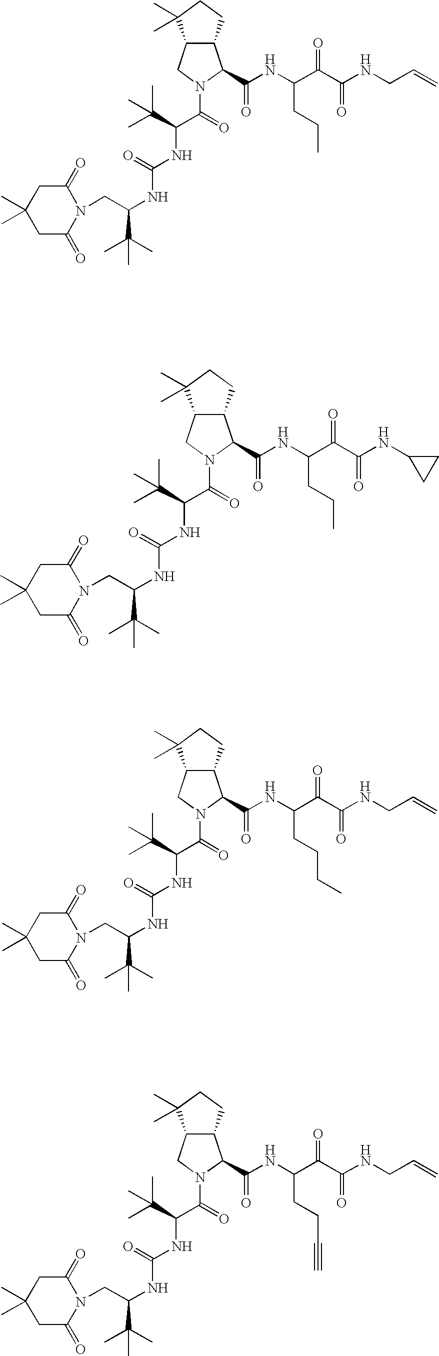 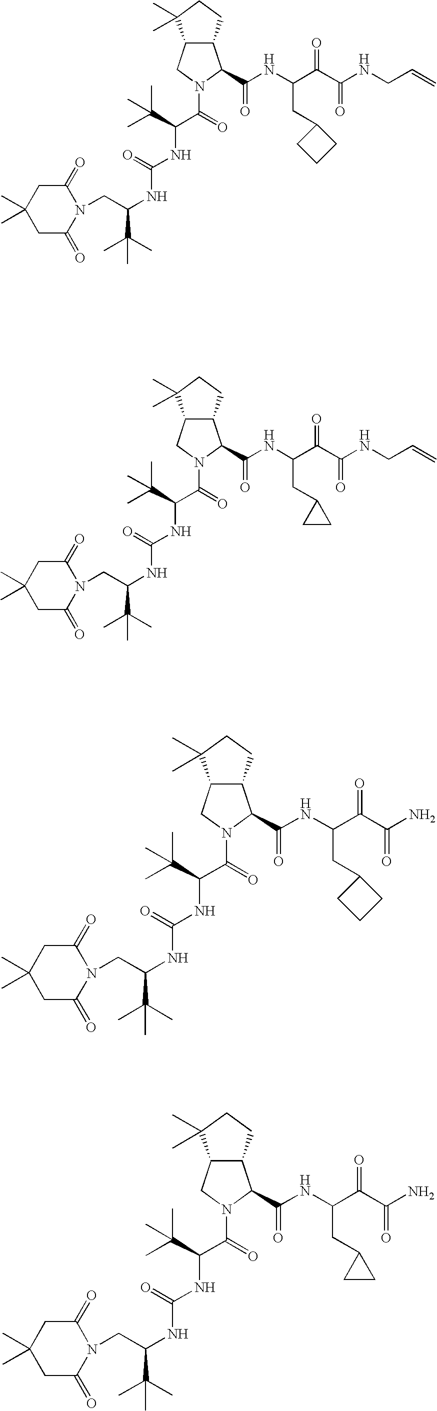 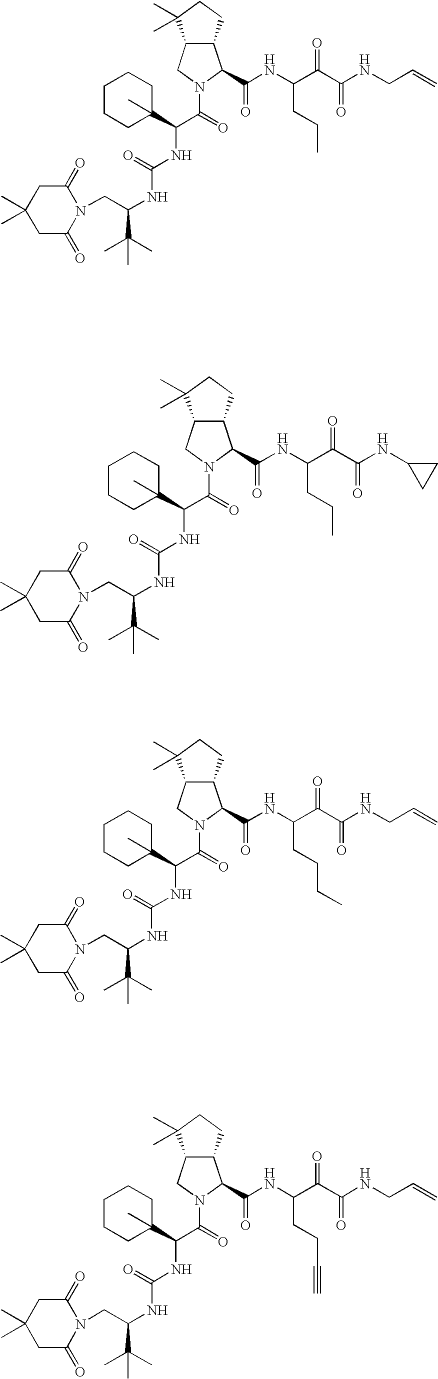 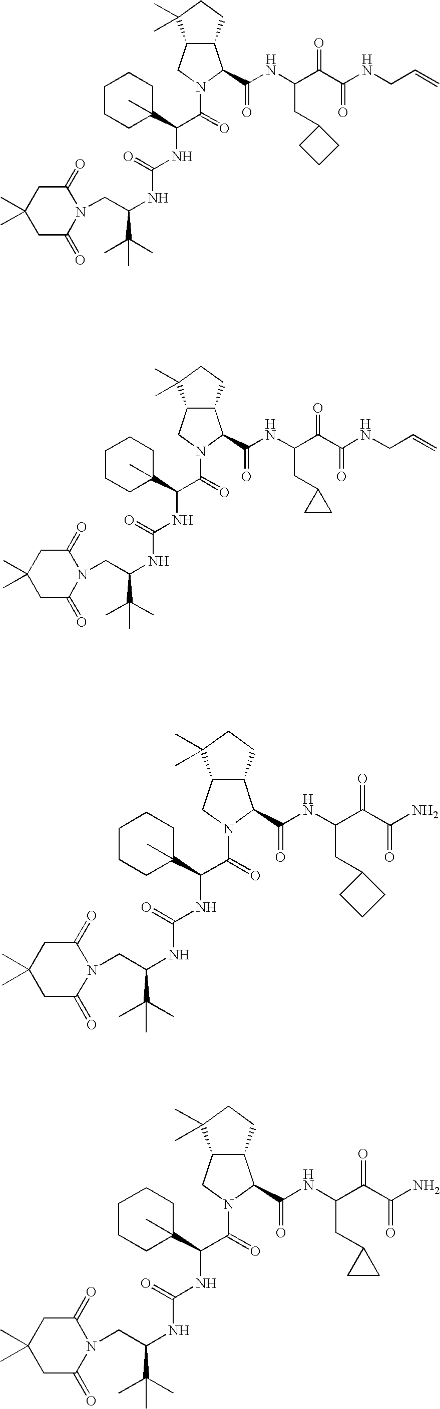 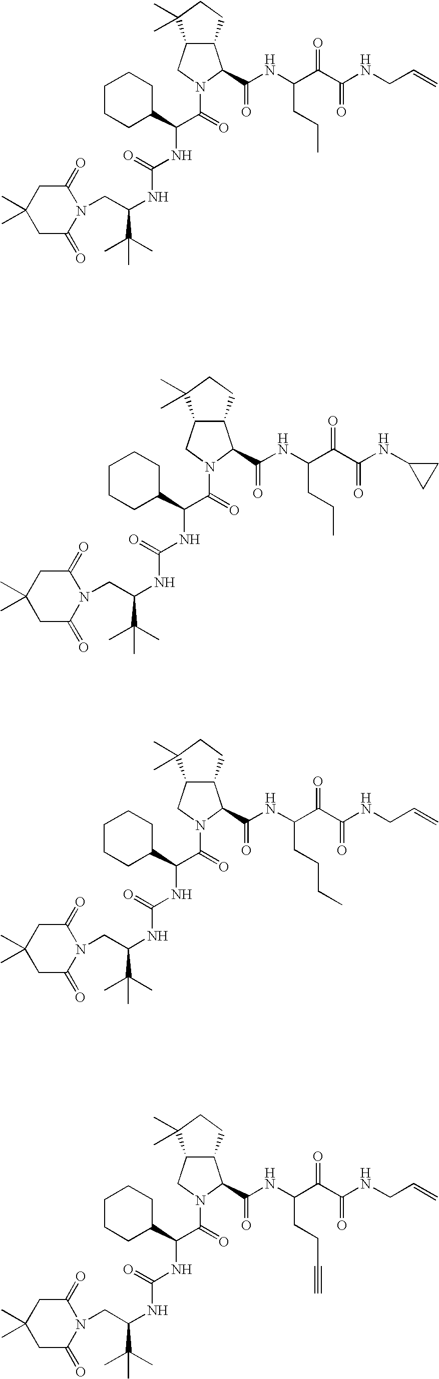 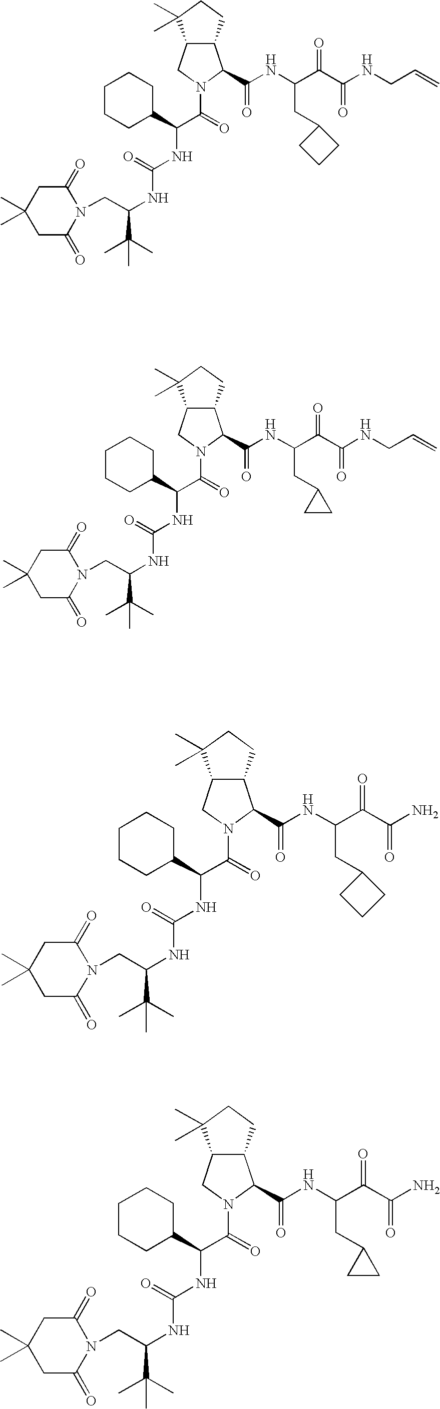 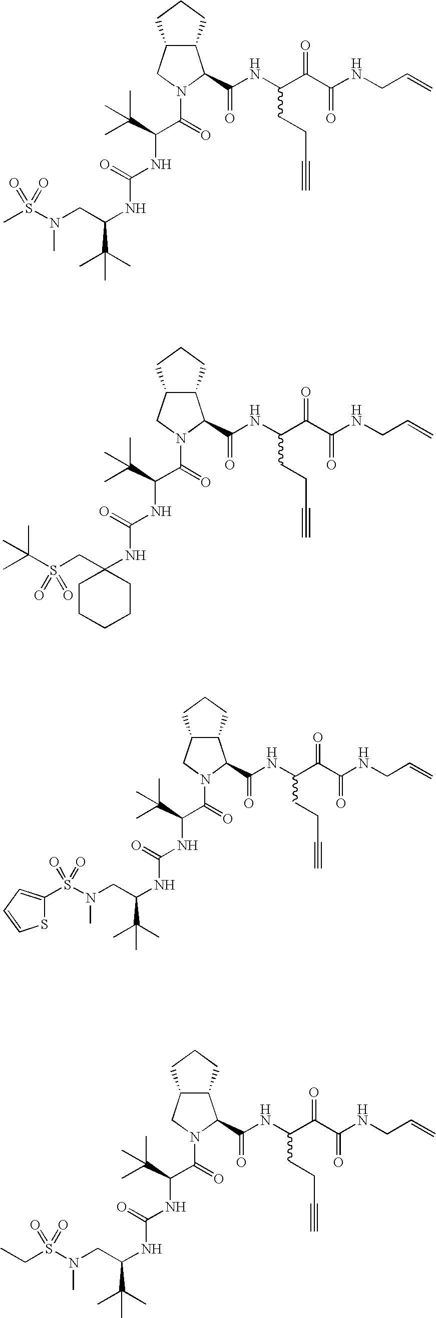 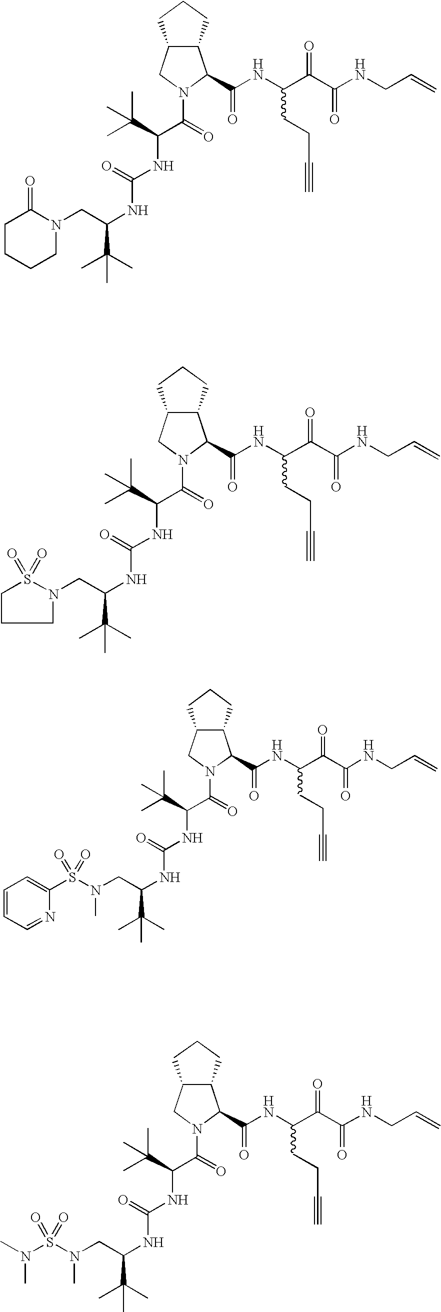 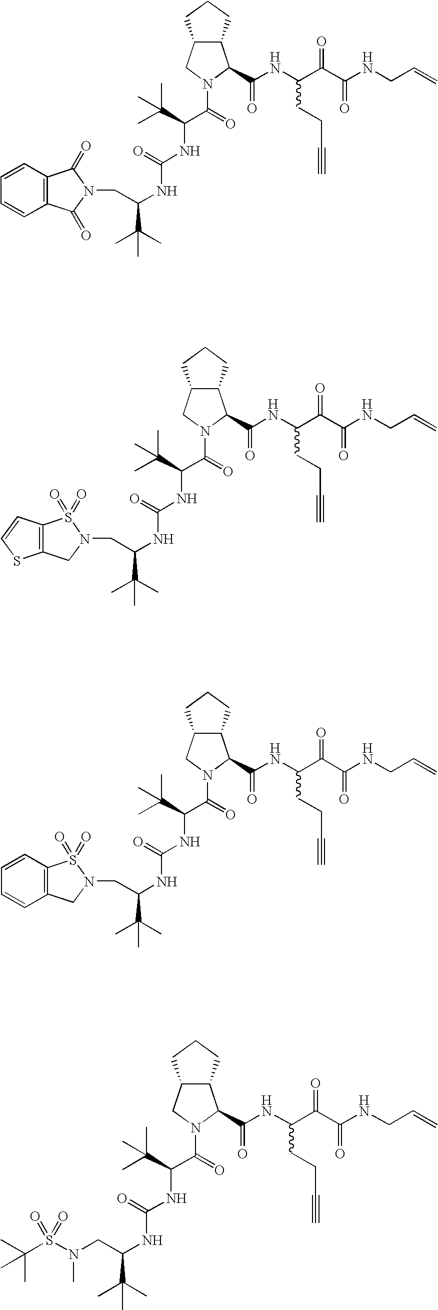 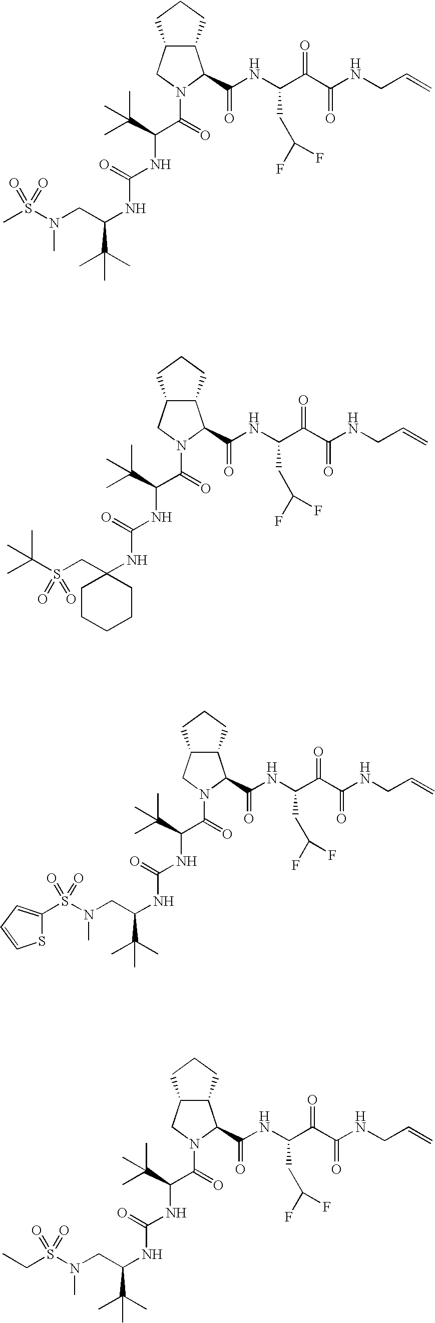 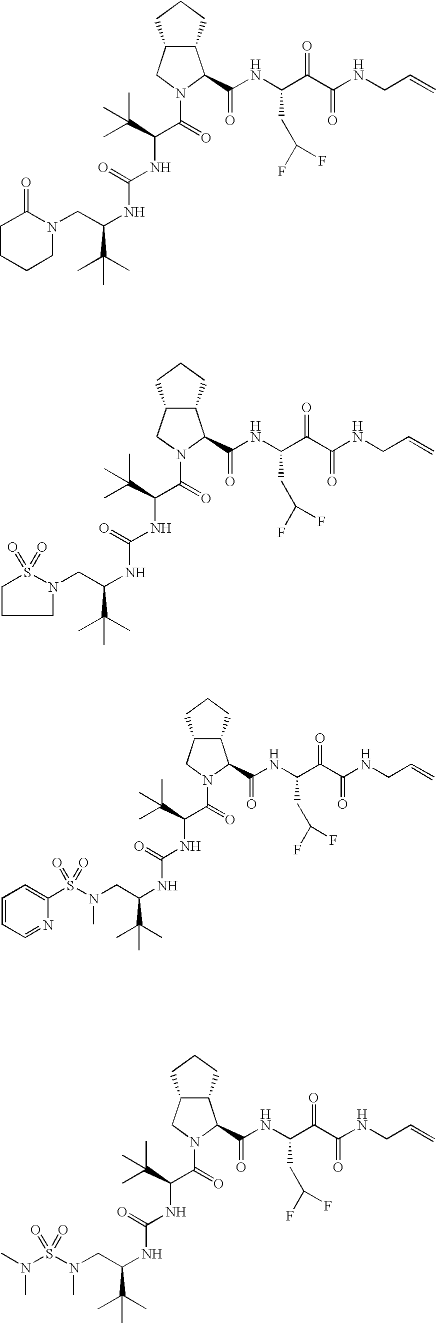 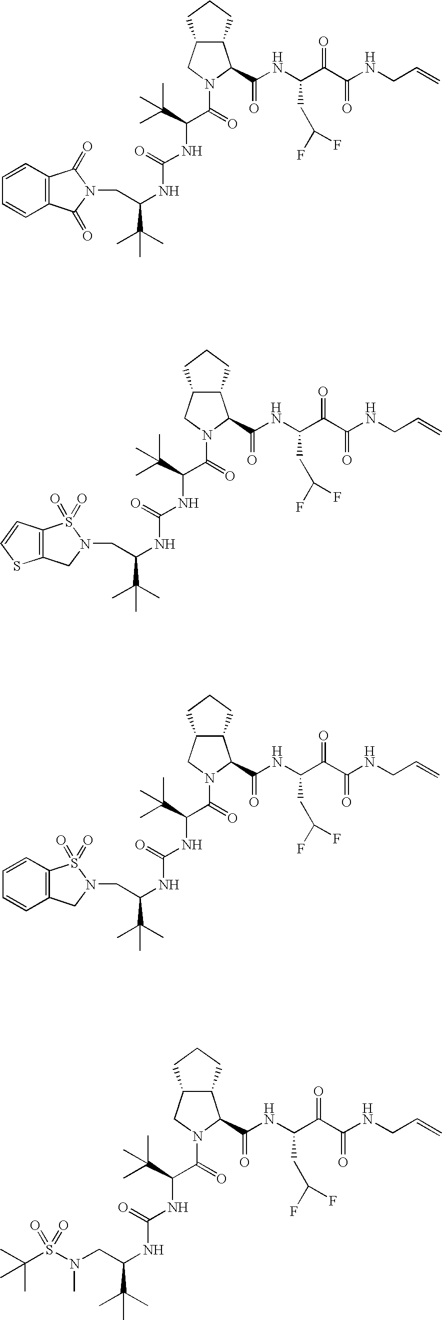 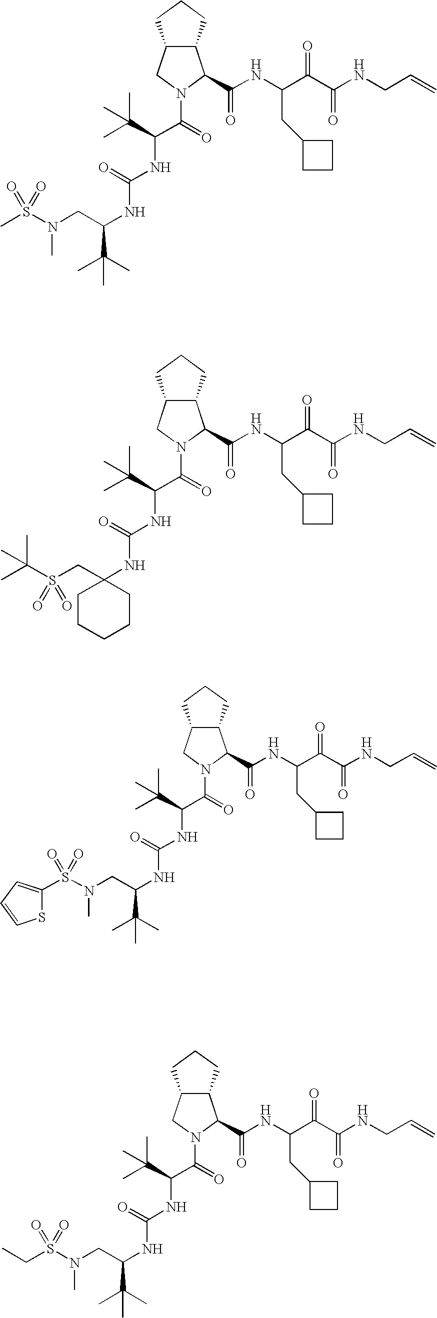 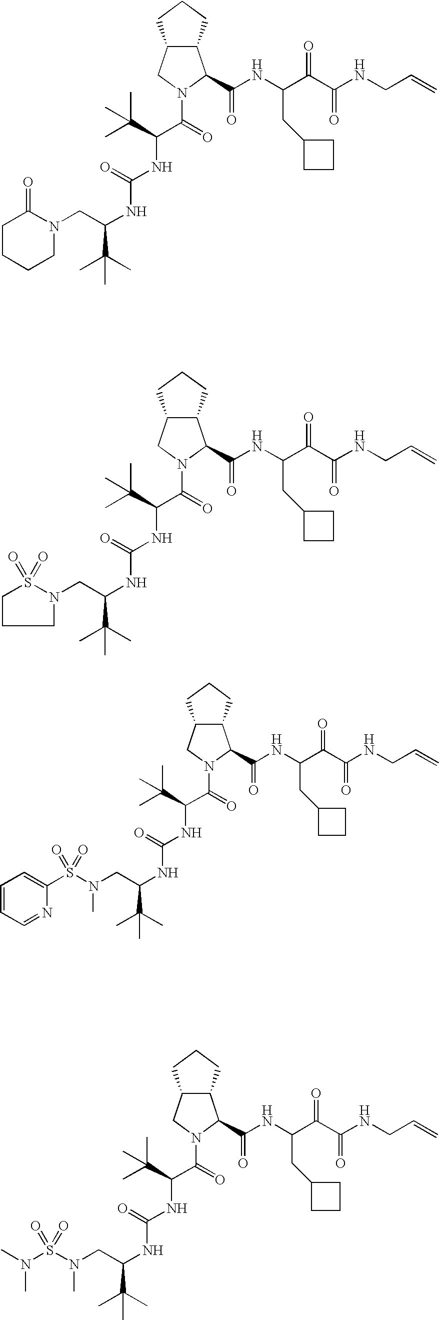 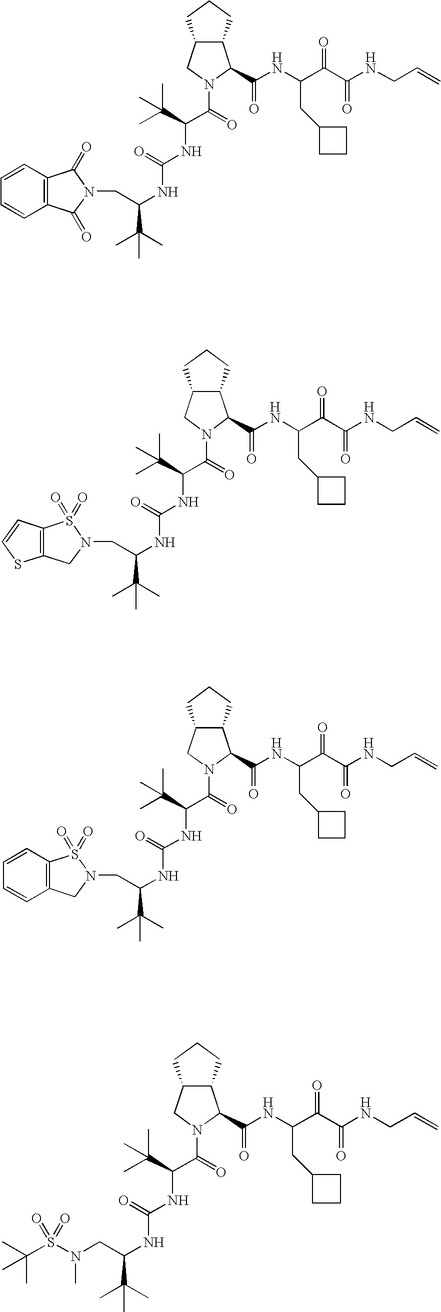 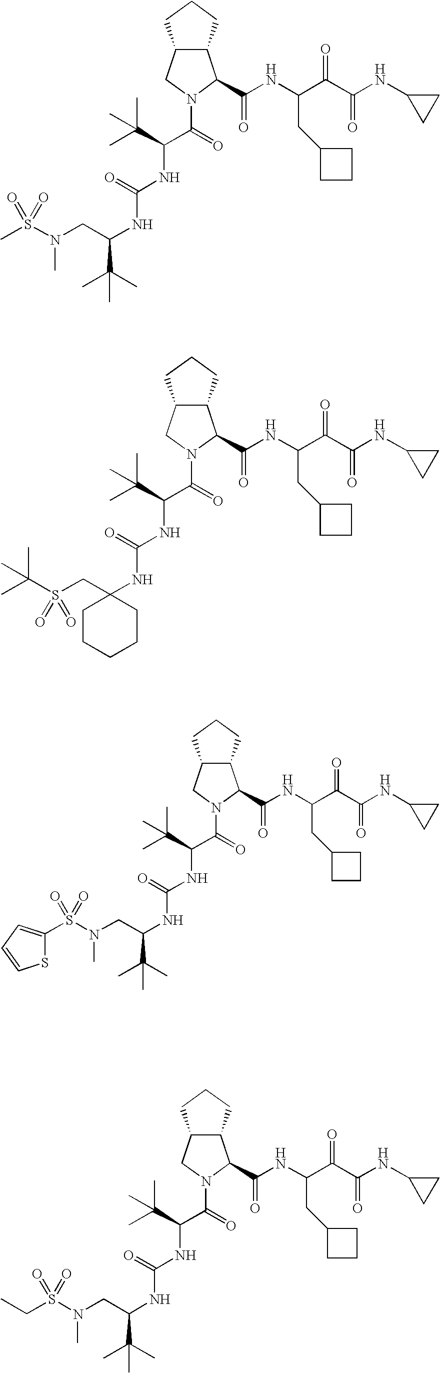 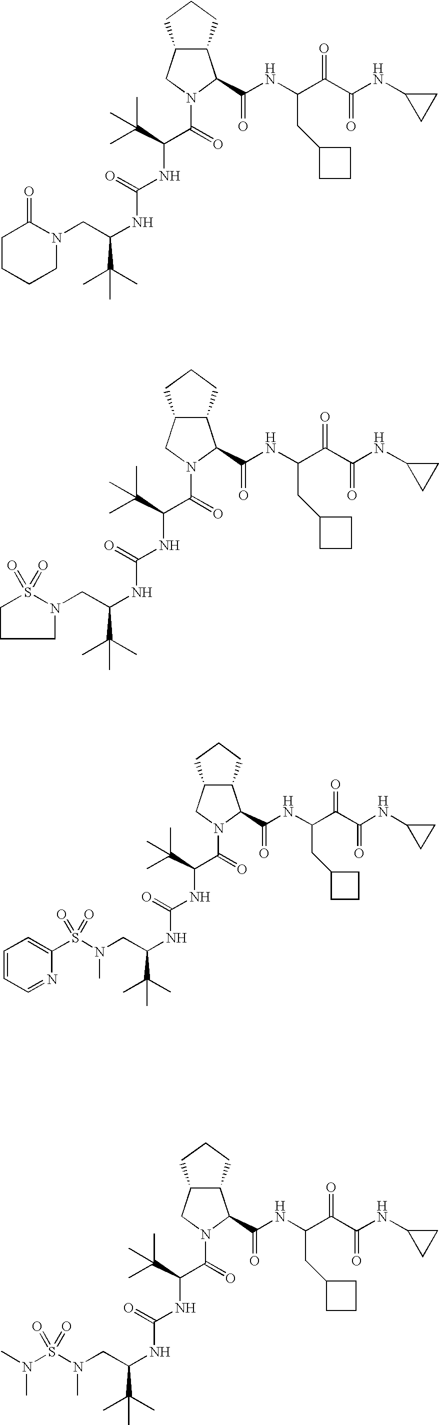 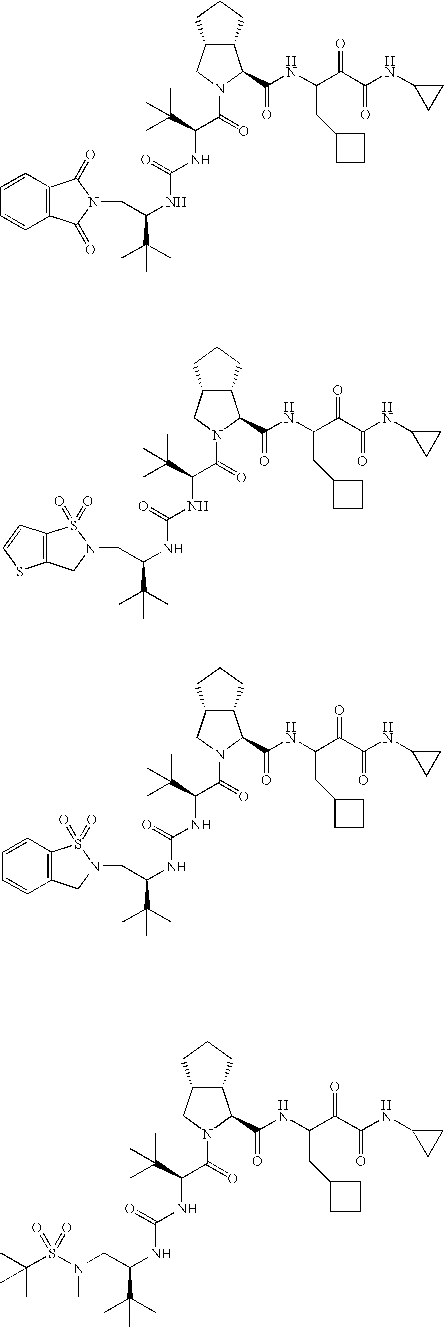 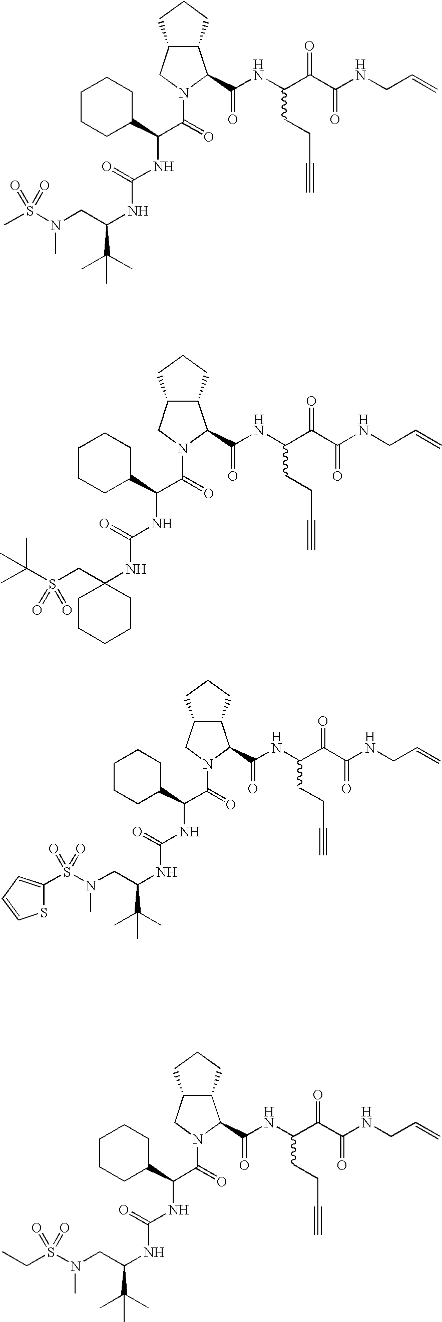 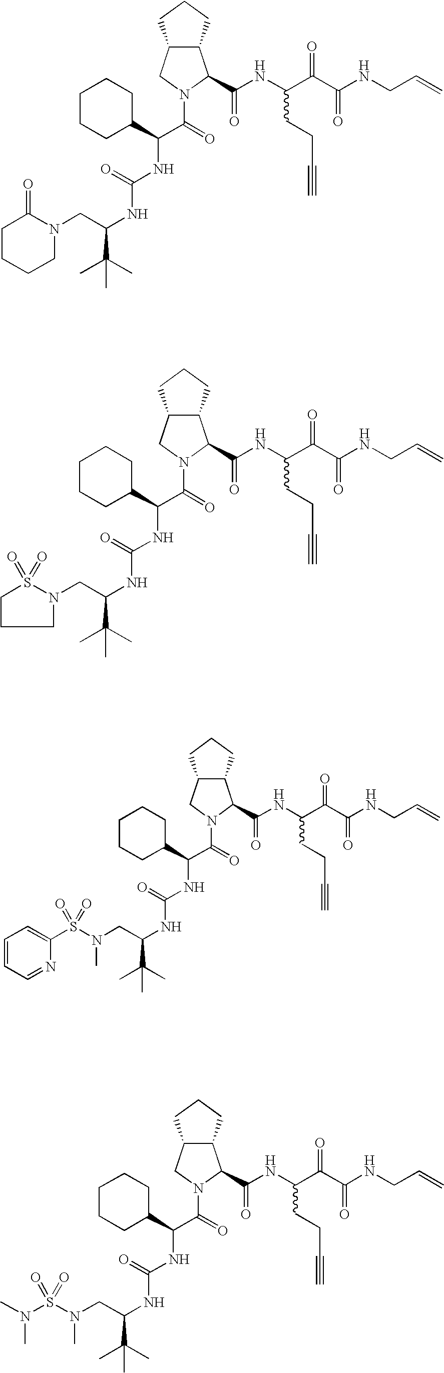 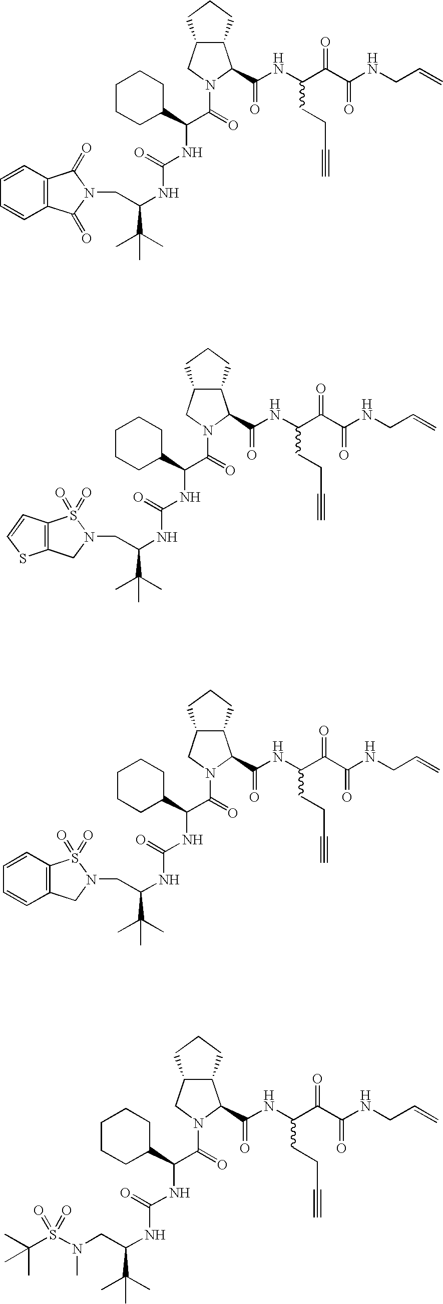 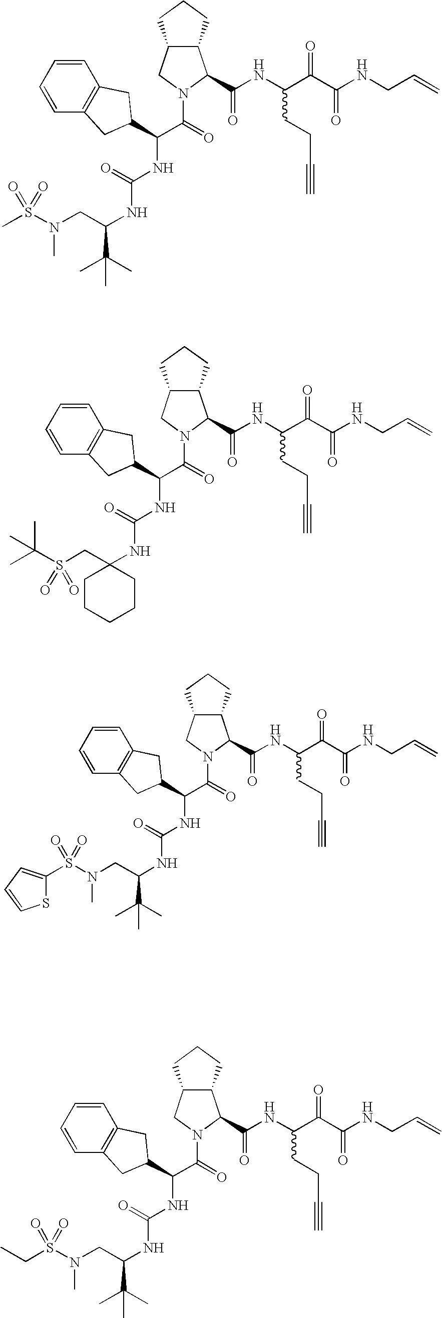 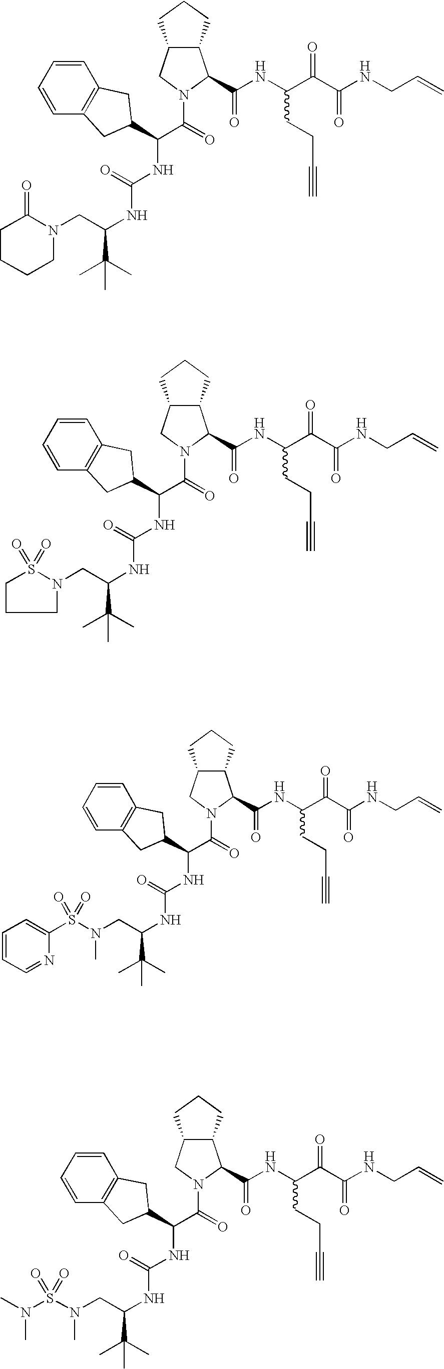 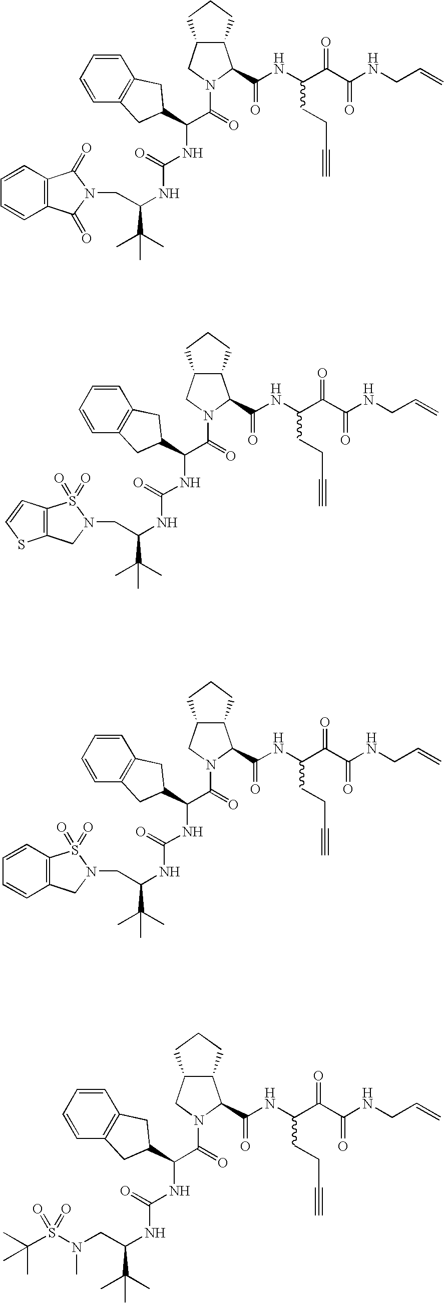 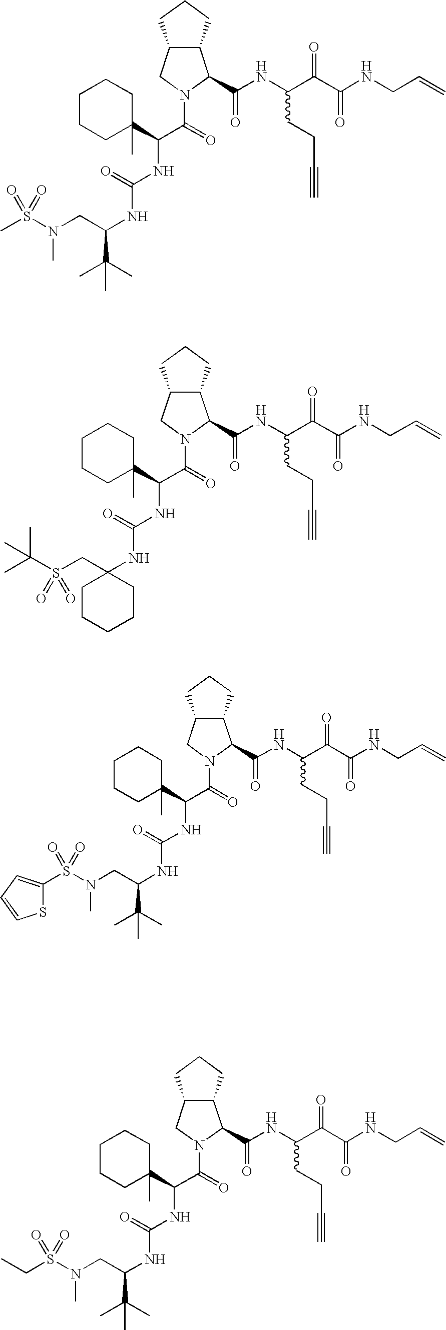 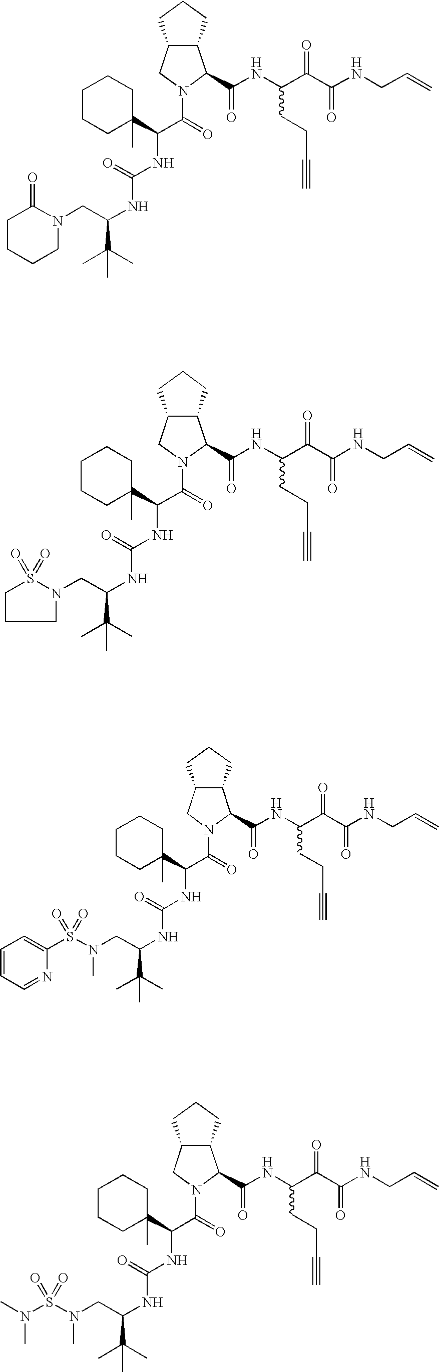 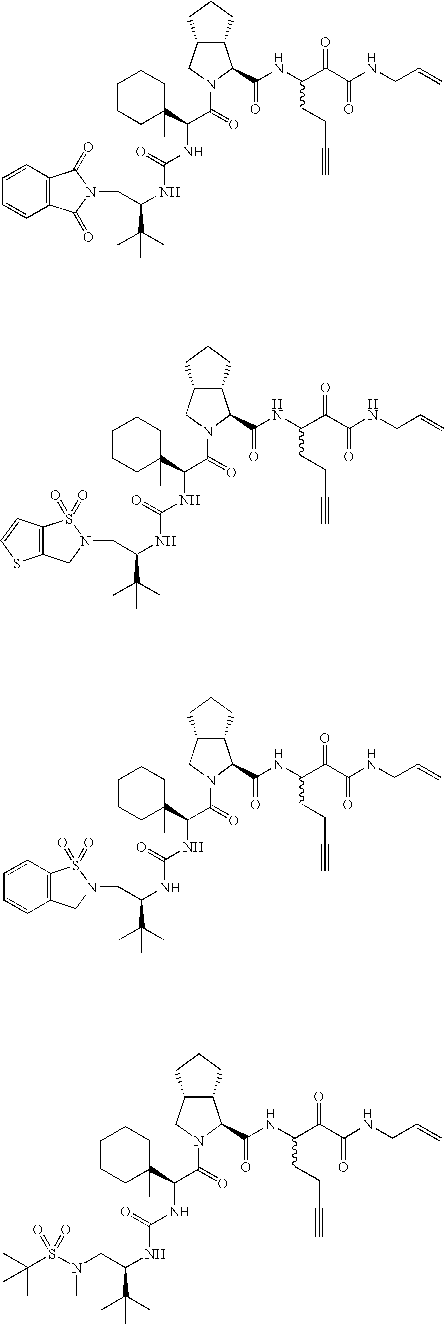
or a pharmaceutically acceptable salt, solvate or ester thereof.

Compounds of formula XVII are disclosed in U.S. Patent Application Publication US 2005/0209164, published on Sep. 22, 2005, and the corresponding PCT publication WO2005/085197, published on Sep. 15, 2005, both of which are incorporated herein by reference.

Non-limiting examples of certain compounds disclosed in these publications are: 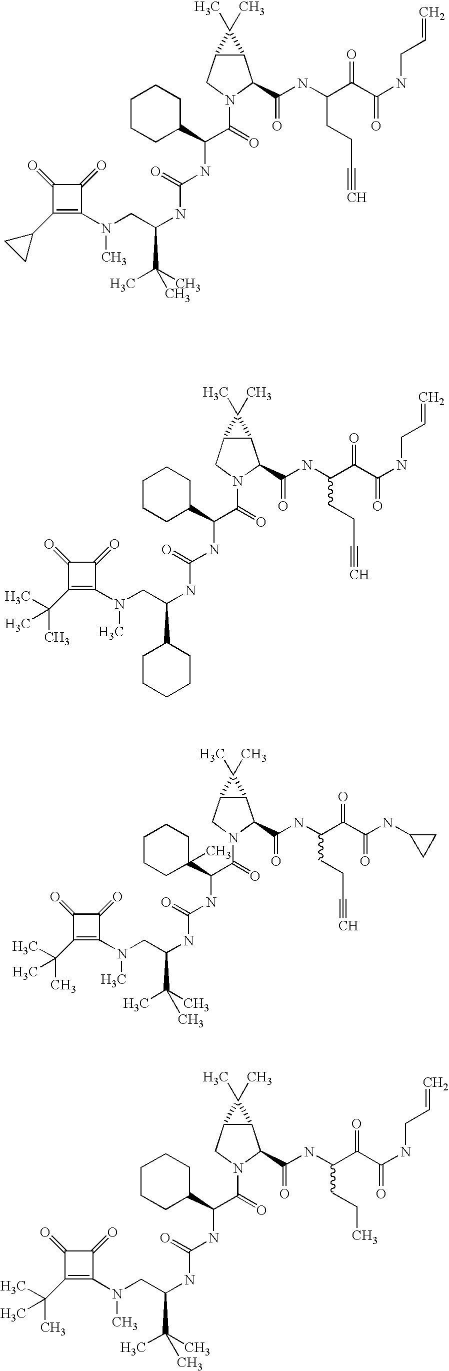 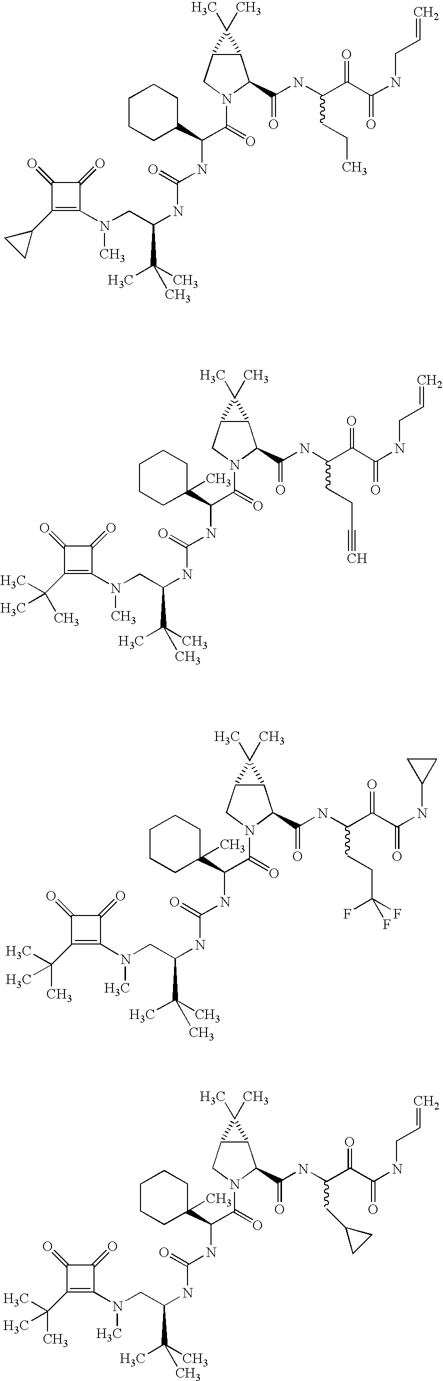 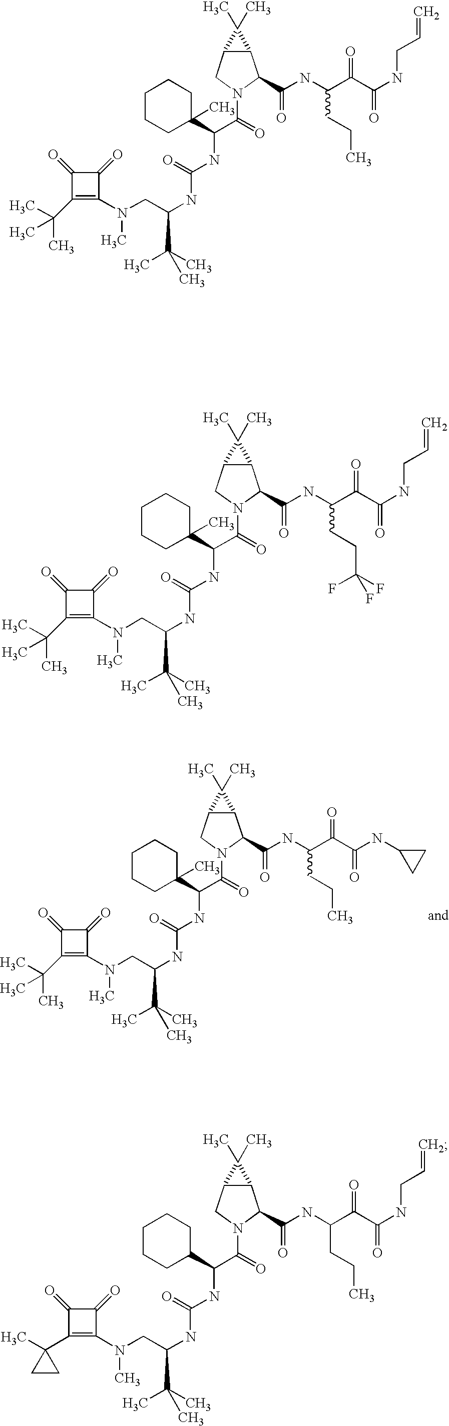
or a pharmaceutically acceptable salt, solvate or ester thereof.

Compounds of formula XVIII are disclosed in U.S. Patent Application Publication US 2006/0046956, published on Mar. 2, 2006, and the corresponding PCT publication WO2006/026352, published Mar. 9, 2006, both of which are incorporated herein by reference.

Non-limiting examples of certain compounds disclosed in these publications are: 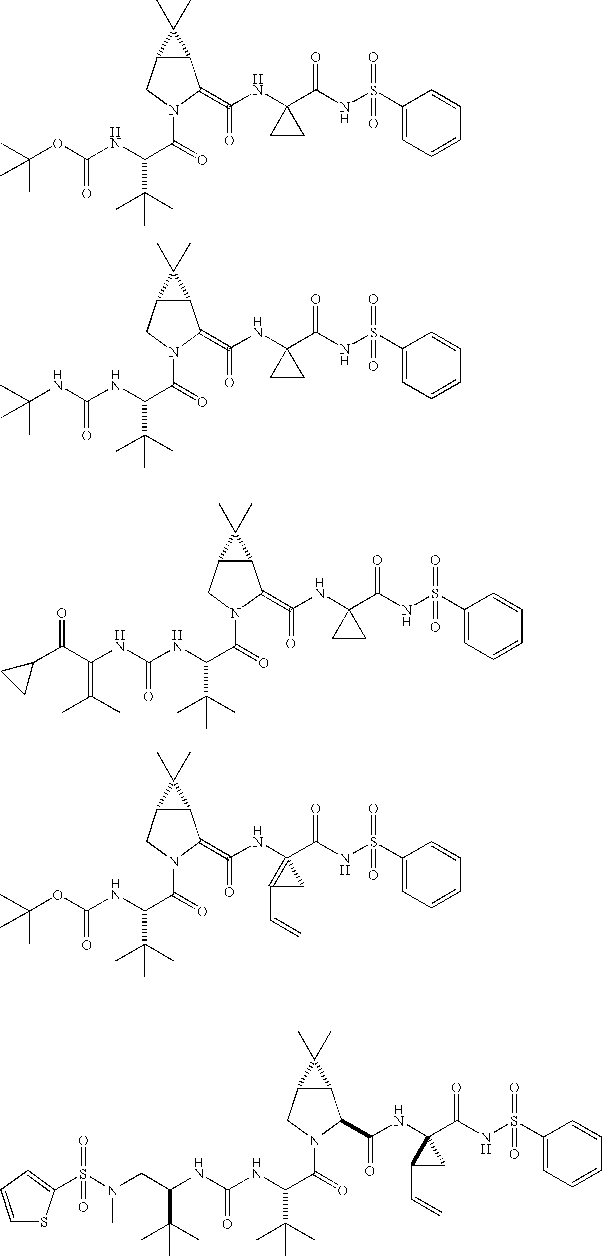 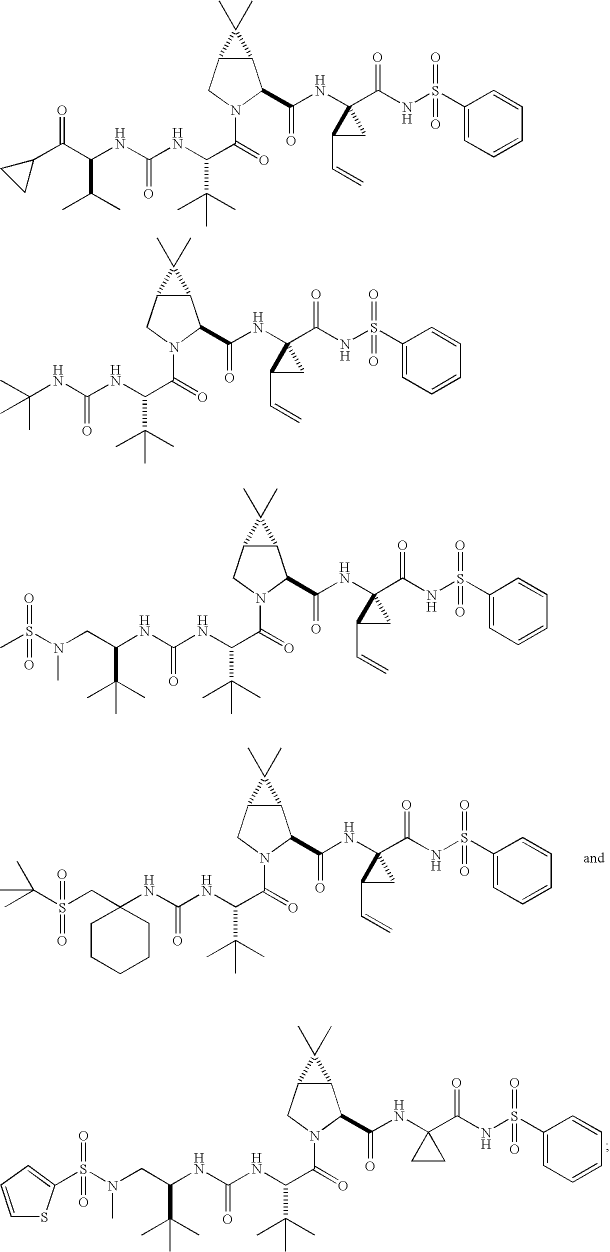
or a pharmaceutically acceptable salt, solvate or ester thereof.

Compounds of formula XIX are disclosed in U.S. Patent Application Publication 2005/0272663, published on Dec. 8, 2005, and the corresponding PCT publication WO2005/113581, published on Dec. 1, 2005, both of which are incorporated herein by reference.

Non-limiting examples of certain compounds disclosed in these publications are: 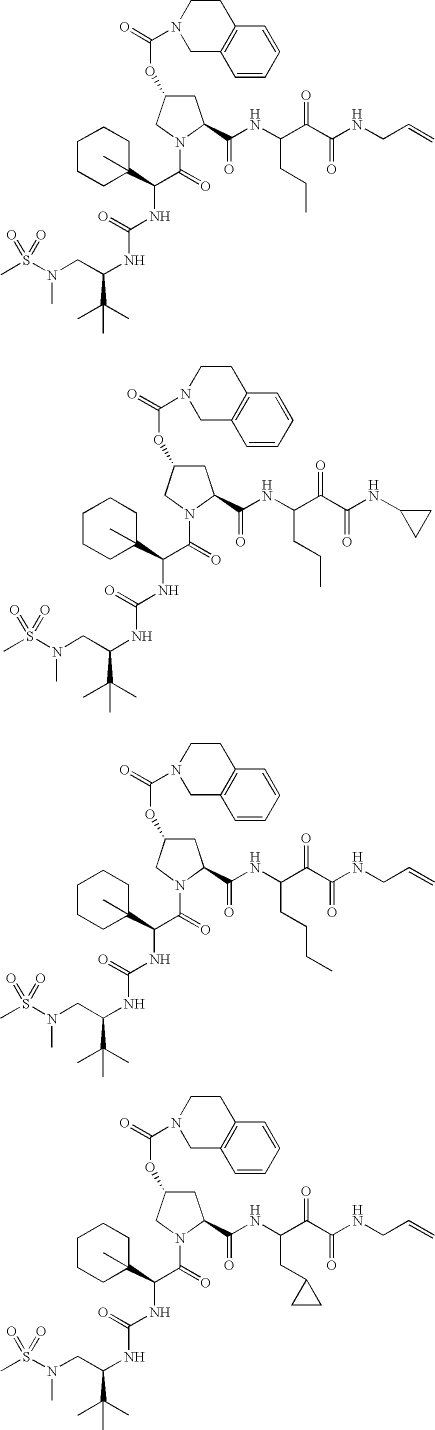 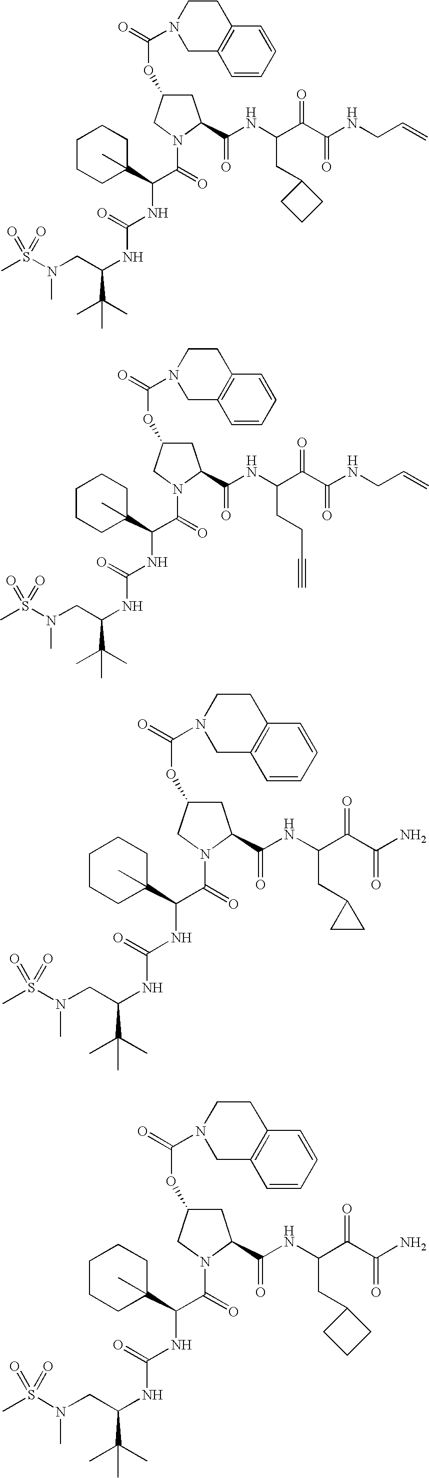 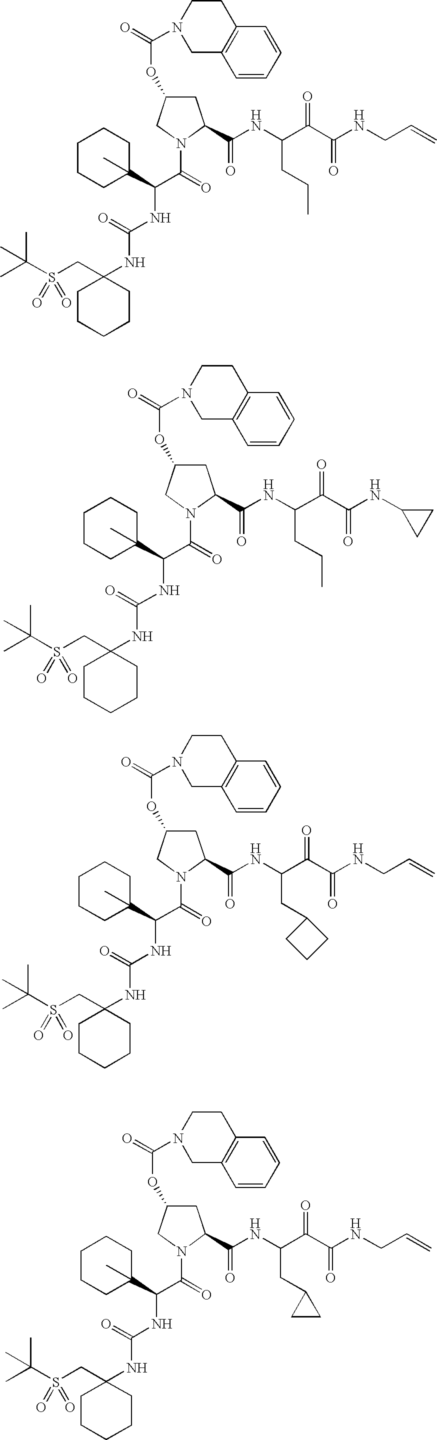 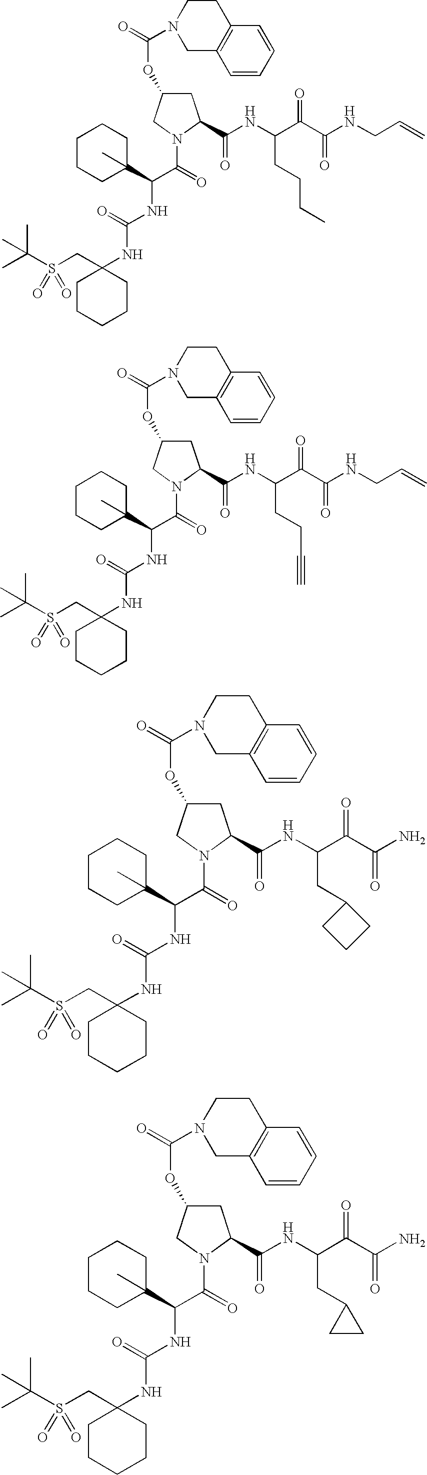 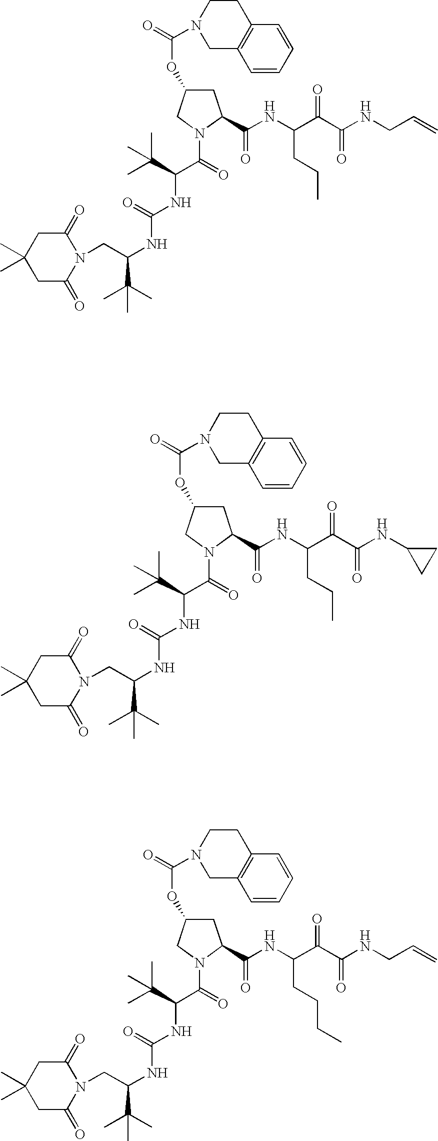 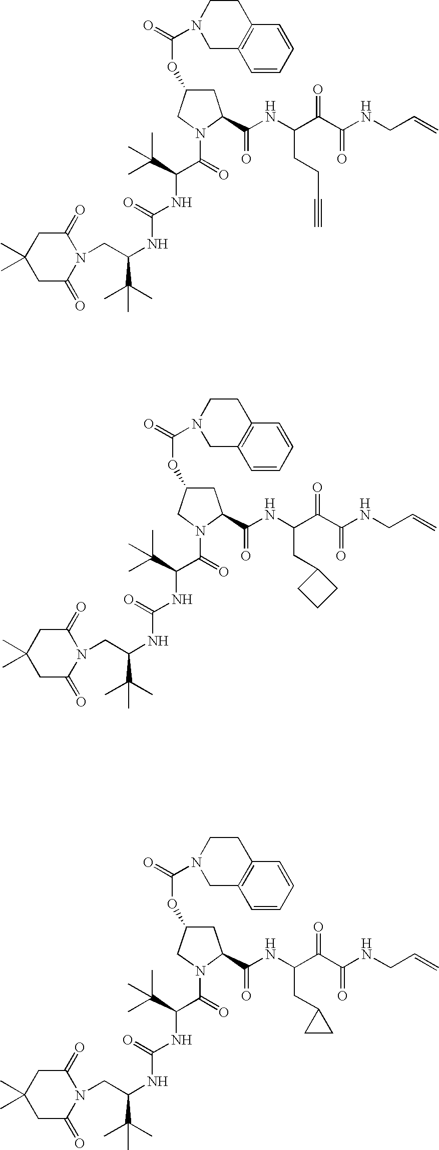 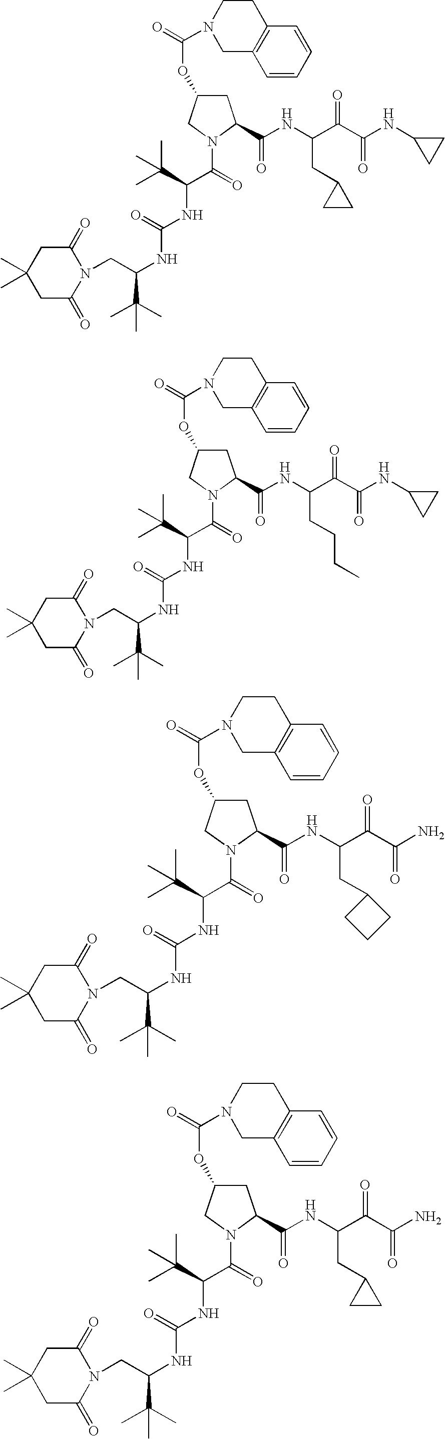 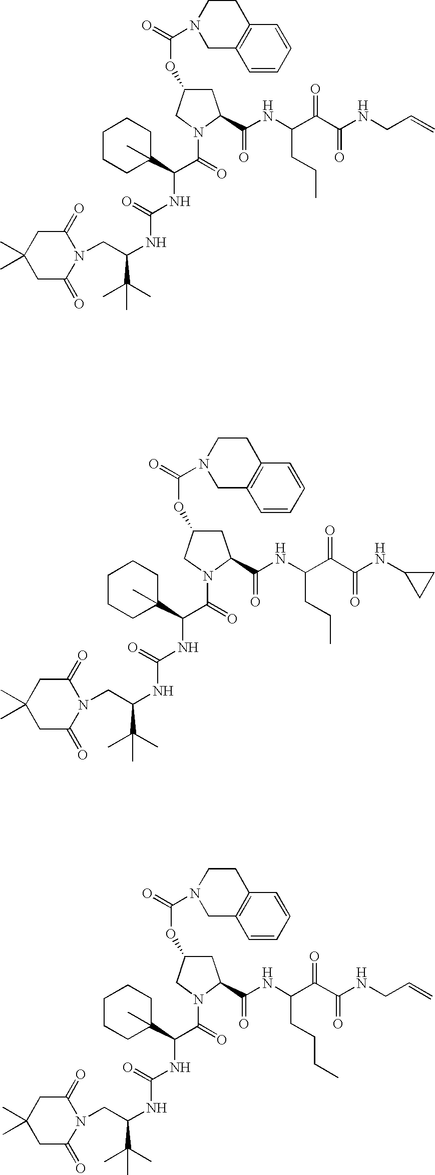 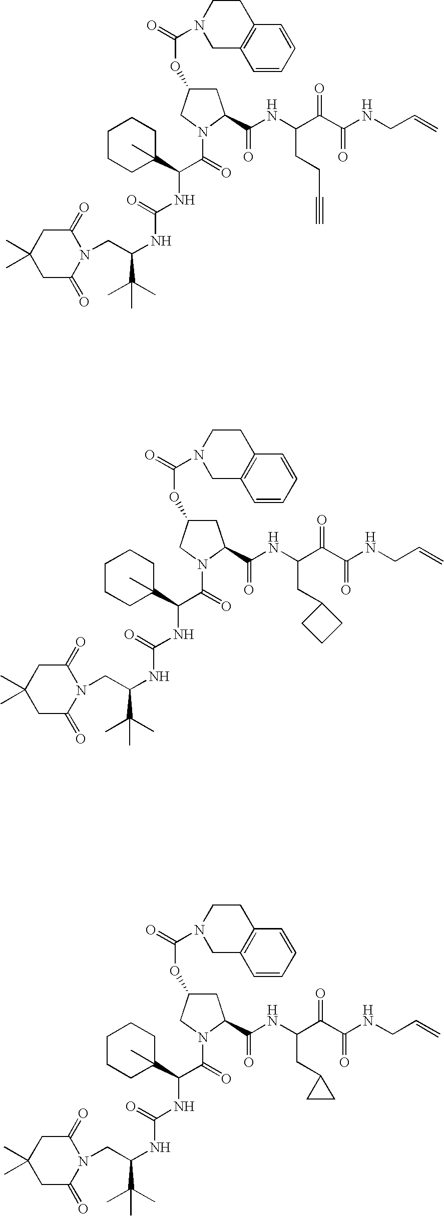 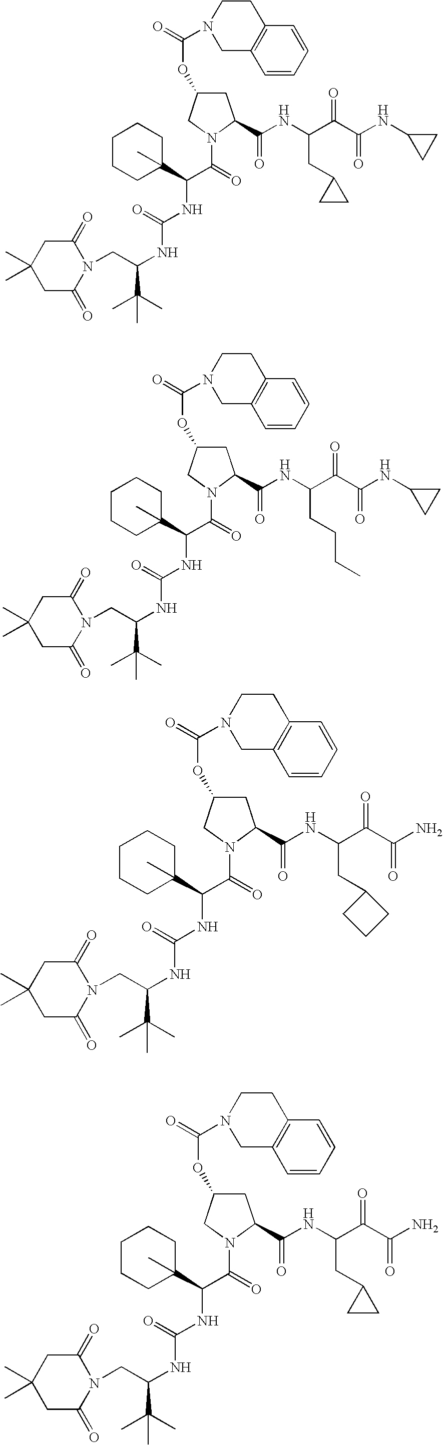 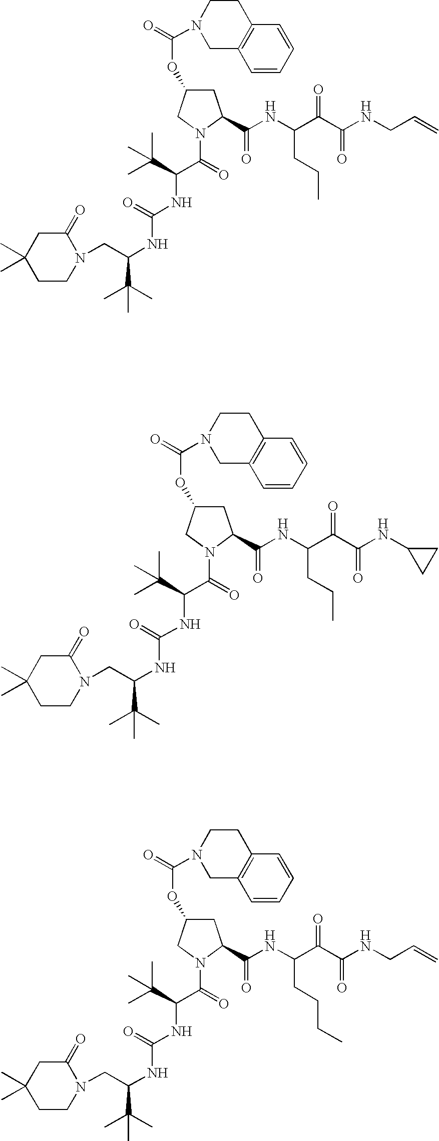 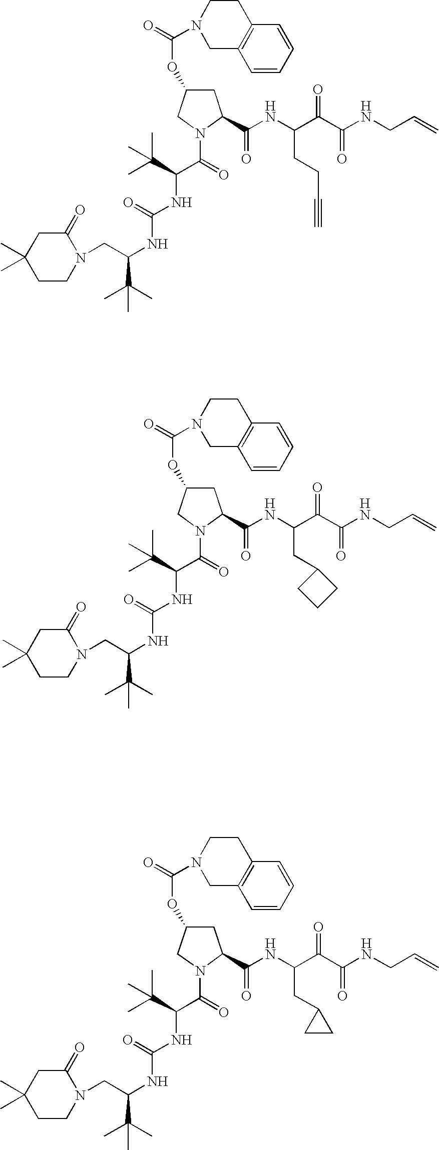 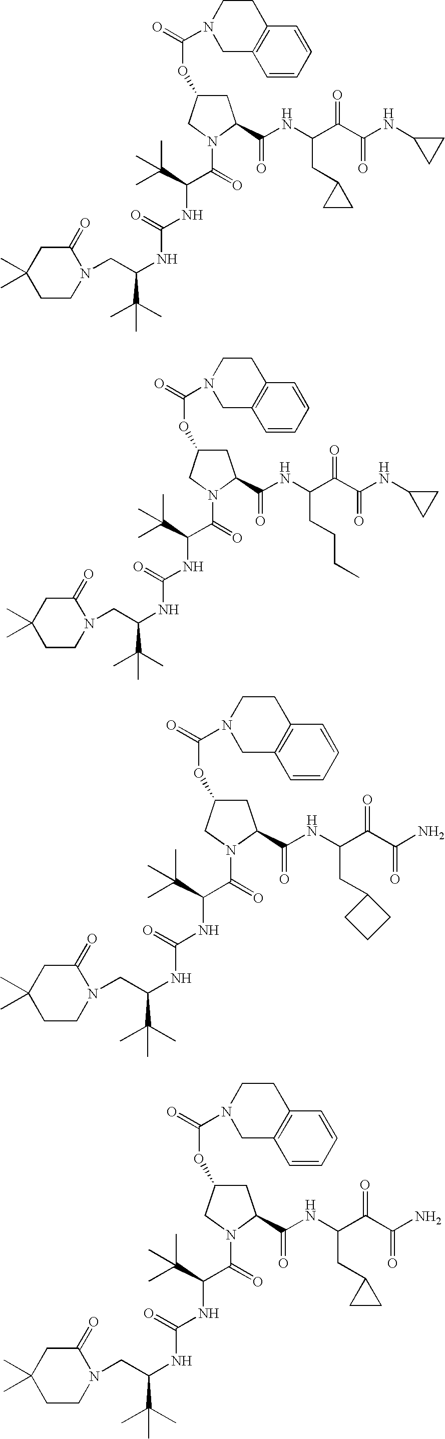 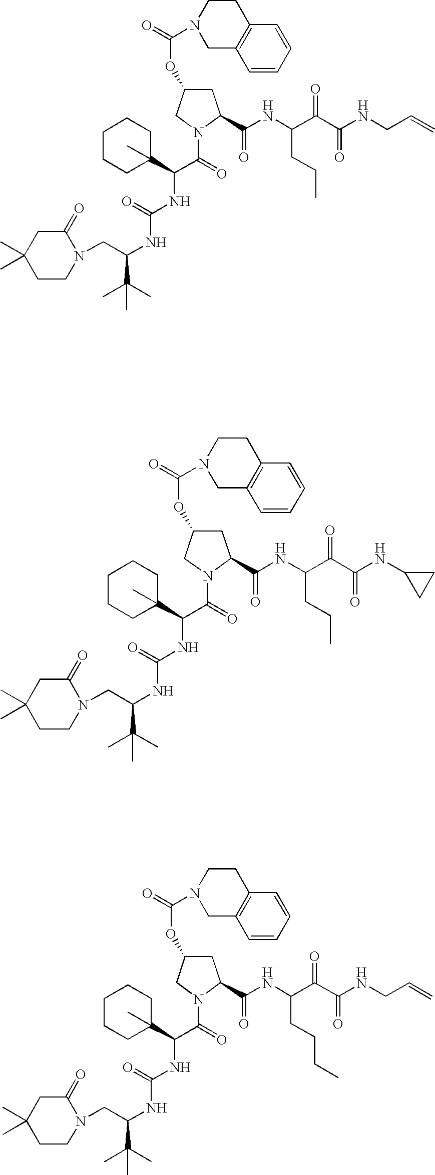 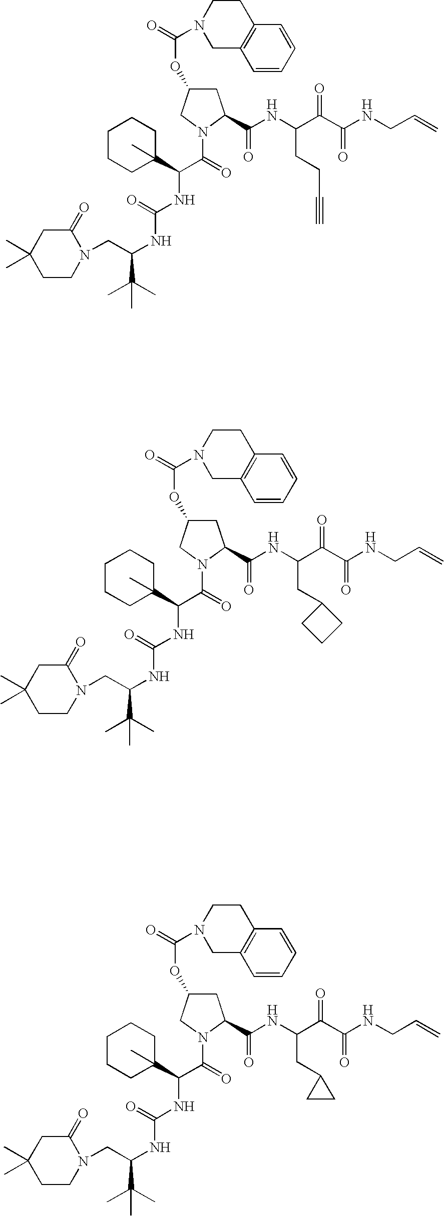 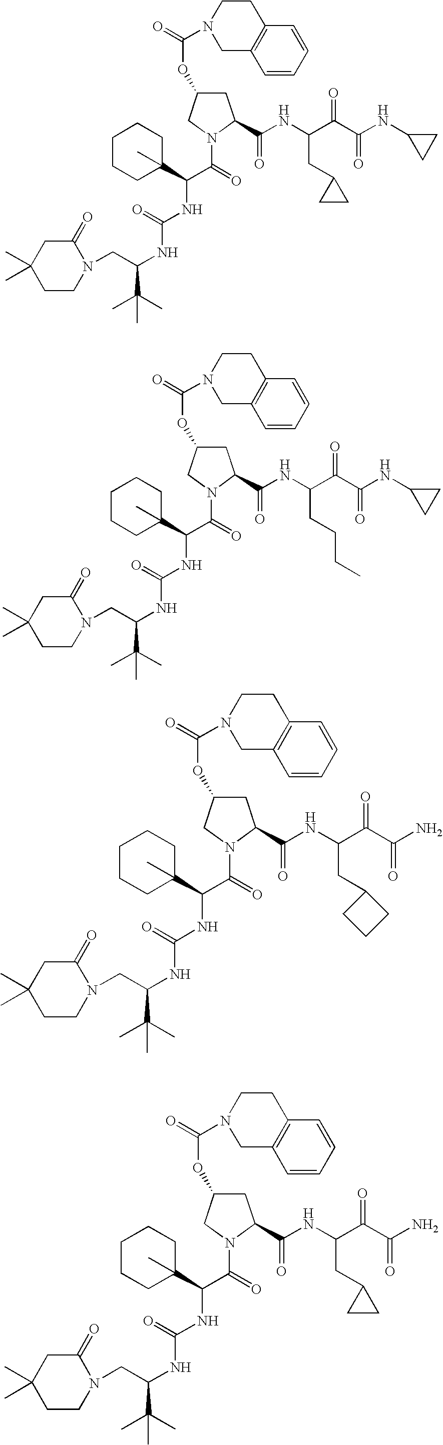 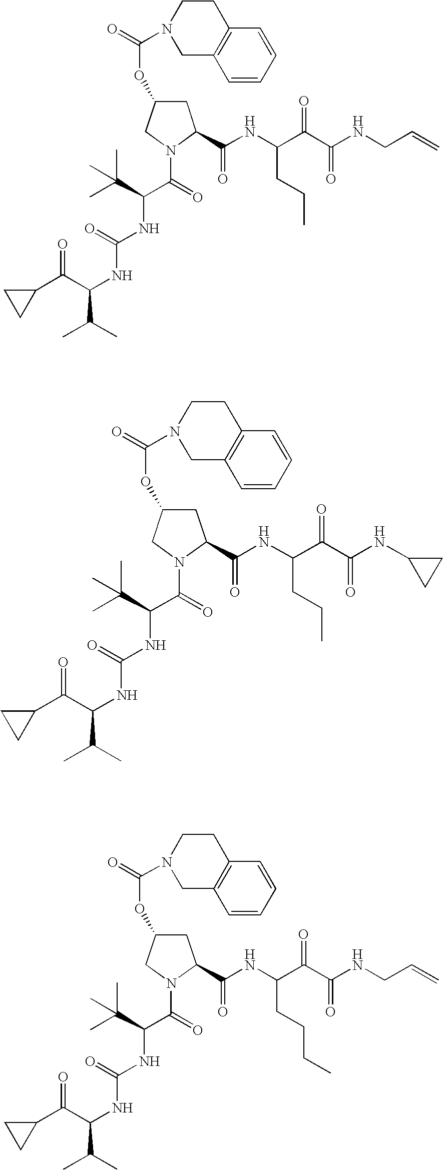 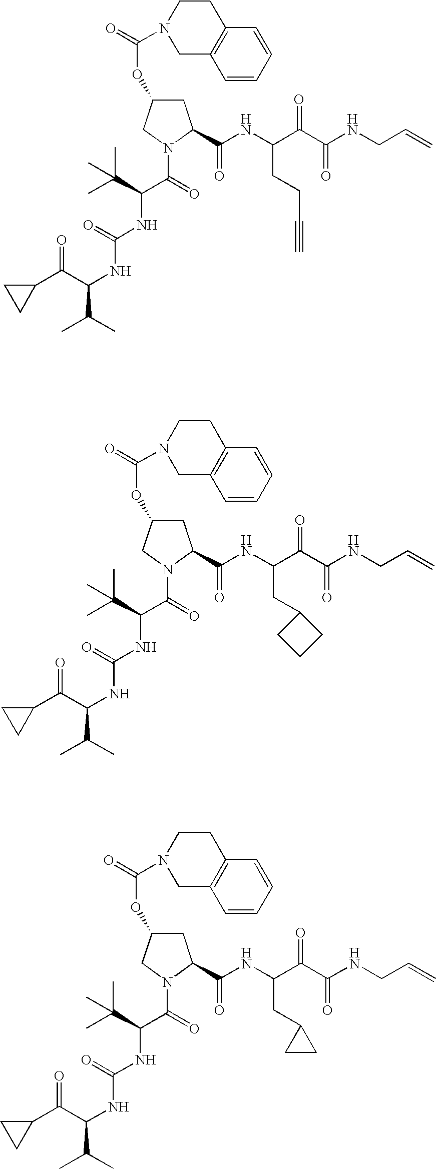 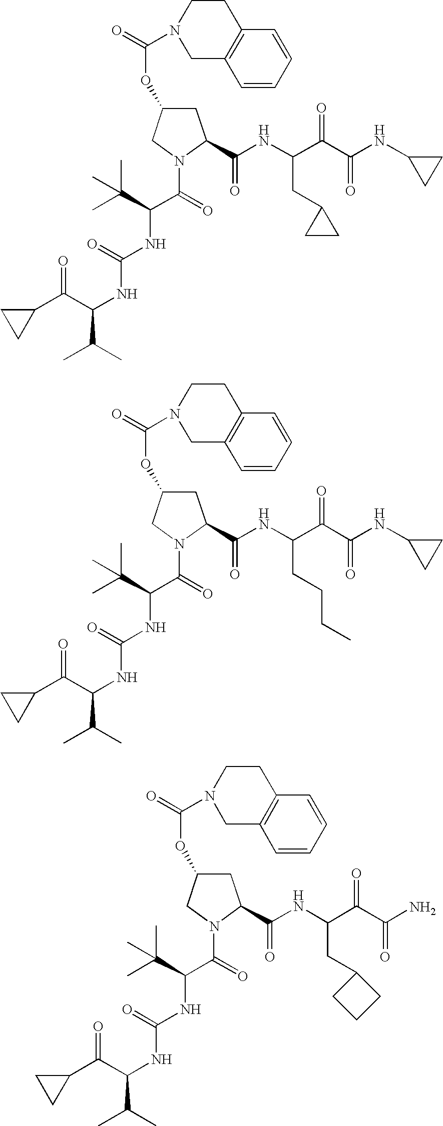 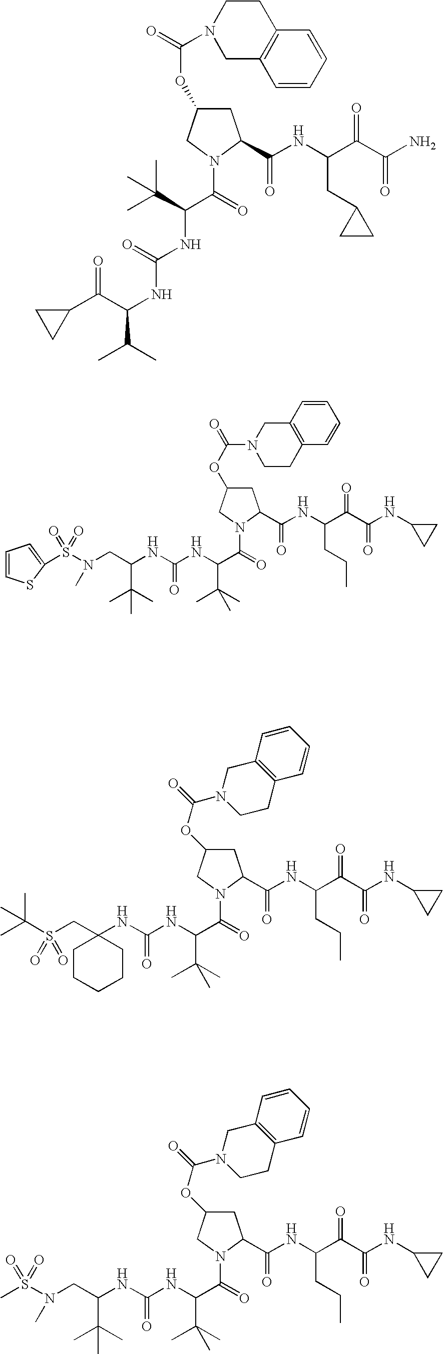 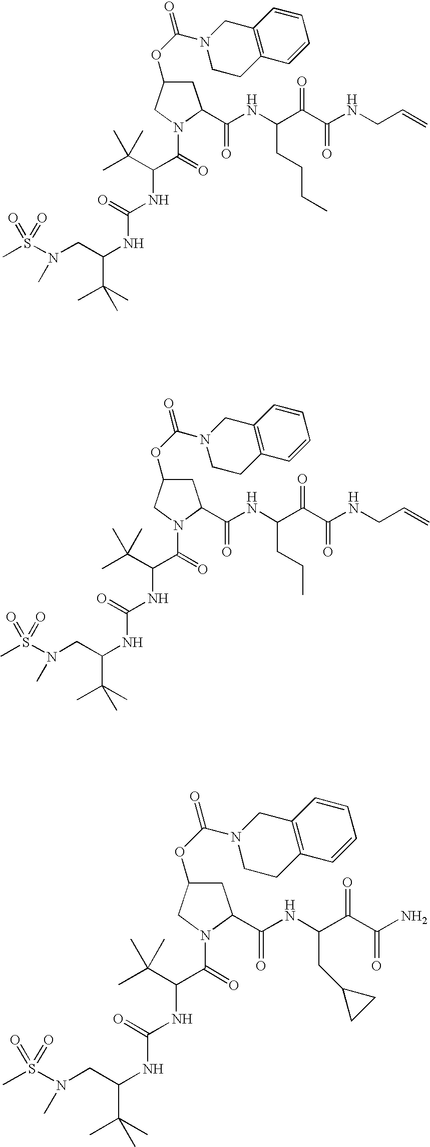 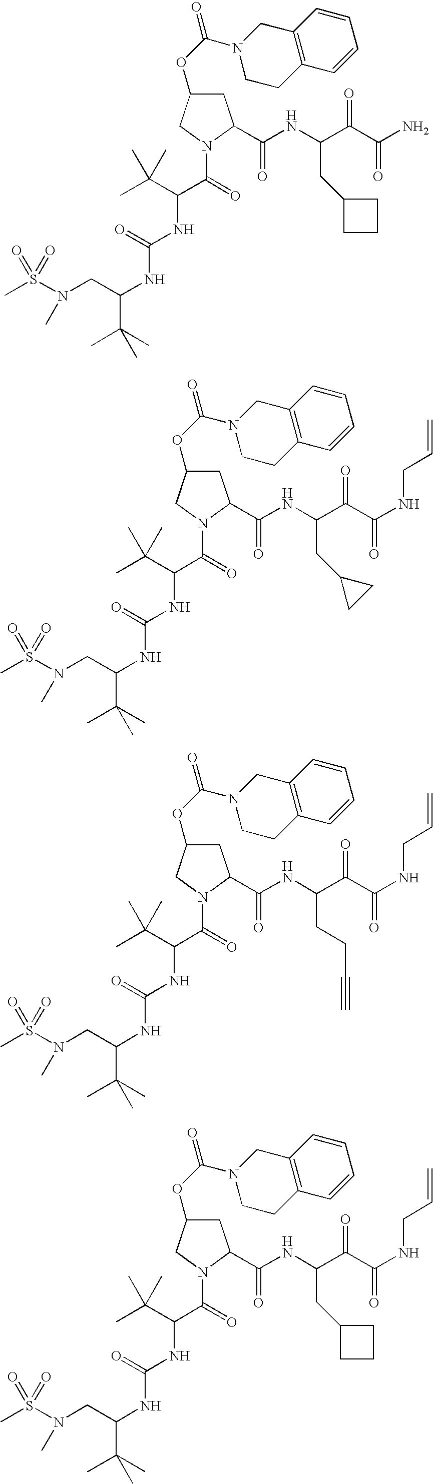 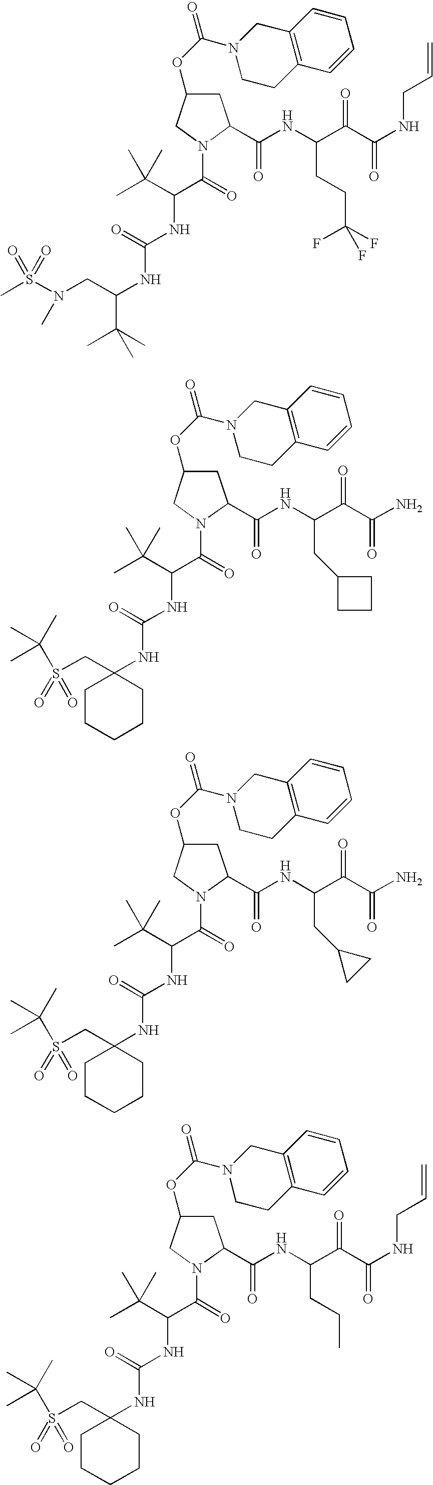 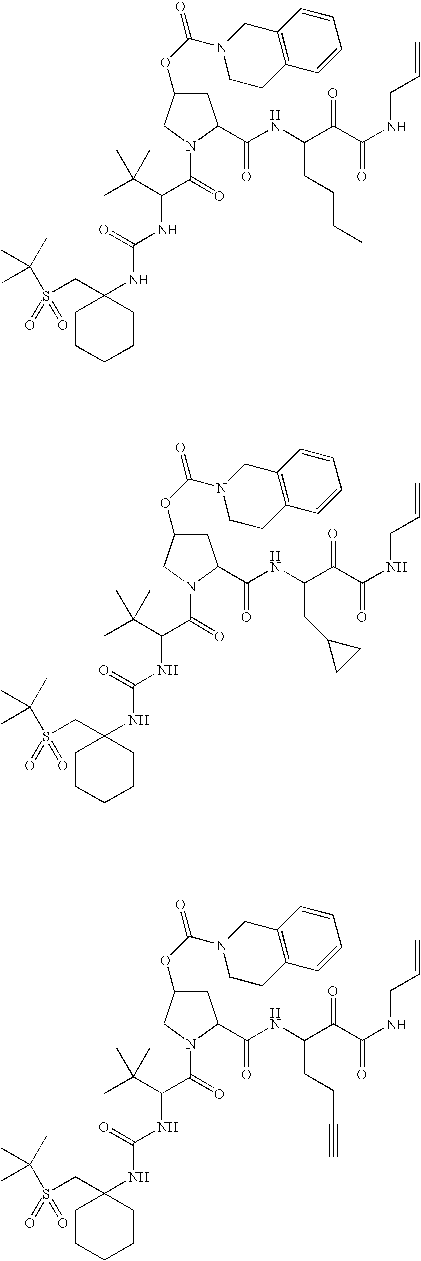 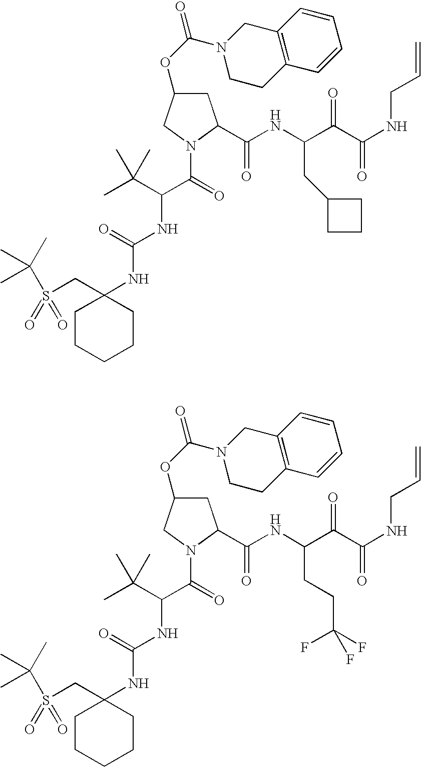
or a pharmaceutically acceptable salt, solvate or ester thereof.

Compounds of formula (XX) have been disclosed in PCT International Patent Publication WO00/09558 published on Feb. 24, 2000, which publication is incorporated herein by reference.

Compounds of formula (XXI) have been disclosed in PCT International Patent Publication WO00/09543 published on Feb. 24, 2000, which publication is incorporated herein by reference.

Compounds of formula (XXII) have been disclosed in U.S. Pat. No. 6,608,027 dated Aug. 19, 2003 and in PCT International Patent Publication WO00/59929 published on Oct. 12, 2000. Both of these publications are incorporated herein by reference.

Compounds of formula (XXIII) have been disclosed in PCT International Patent Publication WO002/18369 published on Mar. 7, 2002 which publication is incorporated herein by reference.

Compounds of formula (XXIV) have been disclosed in PCT International Patent Publication WO98/17679 published on Apr. 30, 1998 which publication is incorporated herein by reference.

Compounds of formula (XXV) have been disclosed in PCT International Patent Publication WO98/22496 published on May 28, 1998 which publication is incorporated herein by reference.

Compounds of formula (XXVI) have been disclosed in PCT International Patent Publication WO99/07734 published on Feb. 18, 1999 which publication is incorporated herein by reference.

Isomers of the various compounds of the present invention (where they exist), including enantiomers, stereoisomers, rotamers, tautomers and racemates are also contemplated as being part of this invention. The invention includes d and I isomers in both pure form and in admixture, including racemic mixtures. Isomers can be prepared using conventional techniques, either by reacting optically pure or optically enriched starting materials or by separating isomers of a compound of the present invention. Isomers may also include geometric isomers, e.g., when a double bond is present. Polymorphous forms of the compounds of the present invention, whether crystalline or amorphous, also are contemplated as being part of this invention. The (+) isomers of the present compounds are preferred compounds of the present invention.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by a ¹³C- or ¹⁴C-enriched carbon are also within the scope of this invention.

It will be apparent to one skilled in the art that certain compounds of this invention may exist in alternative tautomeric forms. All such tautomeric forms of the present compounds are within the scope of the invention. Unless otherwise indicated, the representation of either tautomer is meant to include the other. For example, both isomers (1) and (2) are contemplated: 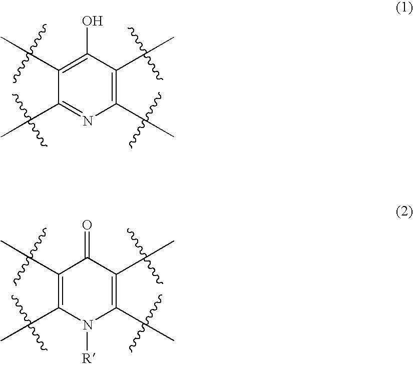
wherein R′ is H or C₁₋₆ unsubstituted alkyl.

Prodrugs and solvates of the compounds of the invention are also contemplated herein. The term “prodrug”, as employed herein, denotes a compound that is a drug precursor which, upon administration to a subject, undergoes chemical conversion by metabolic or chemical processes to yield a compound of formula I or a salt, ester and/or solvate thereof (e.g., a prodrug on being brought to the physiological pH or through enzyme action is converted to the desired drug form). A discussion of prodrugs is provided in T. Higuchi and V. Stella, Pro-drugs as Novel Delivery Systems (1987) Volume 14 of the A.C.S. Symposium Series, and in Bioreversible Carriers in Drug Design, (1987) Edward B. Roche, ed., American Pharmaceutical Association and Pergamon Press, both of which are incorporated herein by reference thereto.

“Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

One or more compounds of the invention may also exist as, or optionally converted to, a solvate. Preparation of solvates is generally known. Thus, for example, M. Caira et al, J. Pharmaceutical Sci., 93(3), 601-611 (2004) describe the preparation of the solvates of the antifungal fluconazole in ethyl acetate as well as from water. Similar preparations of solvates, hemisolvate, hydrates and the like are described by E. C. van Tonder et al, AAPS PharmSciTech., 5(1), article 12 (2004); and A. L. Bingham et al, Chem. Commun., 603-604 (2001). A typical, non-limiting, process involves dissolving a compound in desired amounts of the desired solvent (organic or water or mixtures thereof) at a higher than ambient temperature, and cooling the solution at a rate sufficient to form crystals which are then isolated by standard methods. Analytical techniques such as, for example I. R. spectroscopy, show the presence of the solvent (or water) in the crystals as a solvate (or hydrate).

“Effective amount” or “therapeutically effective amount” is meant to describe an amount of a compound or a composition of the present invention effective in inhibiting mitotic kinesins, in particular KSP kinesin activity, and thus producing the desired therapeutic, ameliorative, inhibitory or preventative effect in a suitable subject.

The compounds of the present invention form salts which are also within the scope of this invention. Reference to a compound of the present invention herein is understood to include reference to salts, esters and solvates thereof, unless otherwise indicated. The term “salt(s)”, as employed herein, denotes acidic salts formed with inorganic and/or organic acids, as well as basic salts formed with inorganic and/or organic bases. In addition, when a compound of formula I contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful. Salts of the compounds of the various formulae of the present invention may be formed, for example, by reacting a compound of the present invention with an amount of acid or base, such as an equivalent amount, in a medium such as one in which the salt precipitates or in an aqueous medium followed by lyophilization. Acids (and bases) which are generally considered suitable for the formation of pharmaceutically useful salts from basic (or acidic) pharmaceutical compounds are discussed, for example, by S. Berge et al, Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; in The Orange Book (Food & Drug Administration, Washington, D.C. on their website); and P. Heinrich Stahl, Camille G. Wermuth (Eds.), Handbook of Pharmaceutical Salts: Properties, Selection, and Use, (2002) Int'l. Union of Pure and Applied Chemistry, pp. 330-331. These disclosures are incorporated herein by reference thereto.

Exemplary acid addition salts include acetates, adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides, hydrobromides, hydroiodides, 2-hydroxyethanesulfonates, lactates, maleates, methanesulfonates, methyl sulfates, 2-naphthalenesulfonates, nicotinates, nitrates, oxalates, pamoates, pectinates, persulfates, 3-phenylpropionates, phosphates, picrates, pivalates, propionates, salicylates, succinates, sulfates, sulfonates (such as those mentioned herein), tartarates, thiocyanates, toluenesulfonates (also known as tosylates,) undecanoates, and the like.

Exemplary basic salts include ammonium salts, alkali metal salts such as sodium, lithium, and potassium salts, alkaline earth metal salts such as calcium and magnesium salts, aluminum salts, zinc salts, salts with organic bases (for example, organic amines) such as benzathines, diethylamine, dicyclohexylamines, hydrabamines (formed with N,N-bis(dehydroabietyl) ethylenediamine), N-methyl-D-glucamines, N-methyl-D-glucamides, t-butyl amines, piperazine, phenylcyclohexylamine, choline, tromethamine, and salts with amino acids such as arginine, lysine and the like. Basic nitrogen-containing groups may be quarternized with agents such as lower alkyl halides (e.g. methyl, ethyl, propyl, and butyl chlorides, bromides and iodides), dialkyl sulfates (e.g. dimethyl, diethyl, dibutyl, and diamyl sulfates), long chain halides (e.g. decyl, lauryl, myristyl and stearyl chlorides, bromides and iodides), aralkyl halides (e.g. benzyl and phenethyl bromides), and others.

All such acid salts and base salts are intended to be pharmaceutically acceptable salts within the scope of the invention. All acid and base salts, as well as esters and solvates, are considered equivalent to the free forms of the corresponding compounds for purposes of the invention.

Pharmaceutically acceptable esters of the present compounds include the following groups: (1) carboxylic acid esters obtained by esterification of the hydroxy groups, in which the non-carbonyl moiety of the carboxylic acid portion of the ester grouping is selected from straight or branched chain alkyl (for example, acetyl, n-propyl, t-butyl, or n-butyl), alkoxyalkyl (for example, methoxymethyl), aralkyl (for example, benzyl), aryloxyalkyl (for example, phenoxymethyl), aryl (for example, phenyl optionally substituted with, for example, halogen, C₁₋₄alkyl, or C₁₋₄alkoxy or amino); (2) sulfonate esters, such as alkyl- or aralkylsulfonyl (for example, methanesulfonyl); (3) amino acid esters (for example, L-valyl or L-isoleucyl); (4) phosphonate esters and (5) mono-, di- or triphosphate esters. The phosphate esters may be further esterified by, for example, a C₁₋₂₀ alcohol or reactive derivative thereof, or by a 2,3-di (C₆₋₂₄)acyl glycerol.

In such esters, unless otherwise specified, any alkyl moiety present preferably contains from 1 to 18 carbon atoms, particularly from 1 to 6 carbon atoms, more particularly from 1 to 4 carbon atoms. Any cycloalkyl moiety present in such esters preferably contains from 3 to 6 carbon atoms. Any aryl moiety present in such esters preferably comprises a phenyl group.

In one embodiment, the compounds of the invention can be used to treat cellular proliferation diseases. Such cellular proliferation disease states which can be treated by the compounds, compositions and methods provided herein include, but are not limited to, cancer (further discussed below), hyperplasia, cardiac hypertrophy, autoimmune diseases, fungal disorders, arthritis, graft rejection, inflammatory bowel disease, immune disorders, inflammation, cellular proliferation induced after medical procedures, including, but not limited to, surgery, angioplasty, and the like. Treatment includes inhibiting cellular proliferation. It is appreciated that in some cases the cells may not be in a hyper- or hypoproliferation state (abnormal state) and still require treatment. For example, during wound healing, the cells may be proliferating “normally”, but proliferation enhancement may be desired. Thus, in one embodiment, the invention herein includes application to cells or subjects afflicted or subject to impending affliction with any one of these disorders or states.

The methods provided herein are particularly useful for the treatment of cancer including solid tumors such as skin, breast, brain, colon, gall bladder, thyroid, cervical carcinomas, testicular carcinomas, etc. More particularly, cancers that may be treated by the compounds, compositions and methods of the invention include, but are not limited to:

Cardiac: sarcoma (angiosarcoma, fibrosarcoma, rhabdomyosarcoma, liposarcoma), myxoma, rhabdomyoma, fibroma, lipoma and teratoma;

Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma;

Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma);

Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma);

Liver: hepatoma (hepatocellular carcinoma), cholangiocarcinoma, hepatoblastoma, angiosarcoma, hepatocellular adenoma, hemangioma;

Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors;

Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma);

Gynecological: uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma);

Hematologic: blood (myeloid leukemia (acute and chronic), acute lymphoblastic leukemia, acute and chronic lymphocytic leukemia, myeloproliferative diseases, multiple myeloma, myelodysplastic syndrome), Hodgkin's disease, non-Hodgkin's lymphoma (malignant lymphoma), B-cell lymphoma, T-cell lymphoma, hairy cell lymphoma, Burkett's lymphoma, promyelocytic leukemia;

Skin: malignant melanoma, basal cell carcinoma, squamous cell carcinoma, Karposi's sarcoma, moles dysplastic nevi, lipoma, angioma, dermatofibroma, keloids, psoriasis;

Adrenal glands: neuroblastoma; and

Other tumors: including xenoderoma pigmentosum, keratoctanthoma and thyroid follicular cancer.

As used herein, treatment of cancer includes treatment of cancerous cells, including cells afflicted by any one of the above-identified conditions.

The compounds of the present invention may also be useful in the chemoprevention of cancer. Chemoprevention is defined as inhibiting the development of invasive cancer by either blocking the initiating mutagenic event or by blocking the progression of pre-malignant cells that have already suffered an insult or inhibiting tumor relapse.

The compounds of the present invention may also be useful in inhibiting tumor angiogenesis and metastasis.

The compounds of the present invention may also be useful as antifungal agents, by modulating the activity of the fungal members of the bimC kinesin subgroup, as is described in U.S. Pat. No. 6,284,480.

The present compounds are also useful in combination with one or more other known therapeutic agents and anti-cancer agents. Combinations of the present compounds with other anti-cancer or chemotherapeutic agents are within the scope of the invention. Examples of such agents can be found in Cancer Principles and Practice of Oncology by V. T. Devita and S. Hellman (editors), 6^{th} edition (Feb. 15, 2001), Lippincott Williams & Wilkins Publishers. A person of ordinary skill in the art would be able to discern which combinations of agents would be useful based on the particular characteristics of the drugs and the cancer involved. Such anti-cancer agents include, but are not limited to, the following: estrogen receptor modulators, androgen receptor modulators, retinoid receptor modulators, cytotoxic/cytostatic agents, antiproliferative agents, prenyl-protein transferase inhibitors, HMG-CoA reductase inhibitors and other angiogenesis inhibitors, inhibitors of cell proliferation and survival signaling, apoptosis inducing agents and agents that interfere with cell cycle checkpoints. The present compounds are also useful when co-administered with radiation therapy.

The phrase “estrogen receptor modulators” refers to compounds that interfere with or inhibit the binding of estrogen to the receptor, regardless of mechanism. Examples of estrogen receptor modulators include, but are not limited to, tamoxifen, raloxifene, idoxifene, LY353381, LY117081, toremifene, fulvestrant, 4-[7-(2,2-dimethyl-I-oxopropoxy-4-methyl-2-[4-[2-(1-piperidinyl)ethoxy]phenyl]-2H-1-benzopyran-3-yl]-phenyl-2,2-dimethylpropanoate, 4,4′-dihydroxybenzophenone-2,4-dinitrophenyl-ydrazone, aid SH646.

The phrase “androgen receptor modulators” refers to compounds which interfere or inhibit the binding of androgens to the receptor, regardless of mechanism. Examples of androgen receptor modulators include finasteride and other 5α-reductase inhibitors, nilutamide, flutamide, bicalutamide, liarozole, and abiraterone acetate.

The phrase “retinoid receptor modulators” refers to compounds which interfere or inhibit the binding of retinoids to the receptor, regardless of mechanism. Examples of such retinoid receptor modulators include bexarotene, tretinoin, 13-cis-retinoic acid, 9-cis-retinoic acid, a difluoromethylornithine, ILX23-7553, trans-N-(4′-hydroxyphenyl) retinamide, and N-4-carboxyphenyl retinamide.

The phrase “cytotoxic/cytostatic agents” refer to compounds which cause cell death or inhibit cell proliferation primarily by interfering directly with the cell's functioning or inhibit or interfere with cell mycosis, including alkylating agents, tumor necrosis factors, intercalators, hypoxia activatable compounds, microtubule inhibitors/microtubule-stabilizing agents, inhibitors of mitotic kinesins, inhibitors of kinases involved in mitotic progression, antimetabolites; biological response modifiers; hormonal/anti-hormonal therapeutic agents, haematopoietic growth factors, monoclonal antibody targeted therapeutic agents, monoclonal antibody therapeutics, topoisomerase inhibitors, proteasome inhibitors and ubiquitin ligase inhibitors.

Examples of cytotoxic agents include, but are not limited to, sertenef, cachectin, ifosfamide, tasonermin, lonidamine, carboplatin, altretamine, prednimustine, dibromodulcitol, ranimustine, fotemustine, nedaplatin, oxaliplatin, temozolomide (TEMODAR™ from Schering-Plough Corporation, Kenilworth, N.J.), cyclophosphamide, heptaplatin, estramustine, improsulfan tosilate, trofosfamide, nimustine, dibrospidium chloride, pumitepa, lobaplatin, satraplatin, profiromycin, cisplatin, doxorubicin, irofulven, dexifosfamide, cis-aminedichloro(2-methyl-pyridine)platinum, benzylguanine, glufosfamide, GPX100, (trans, trans, trans)-bis-mu-(hexane-1,6-diamine)-mu-[diamine-platinum(II)]bis[diamine(chloro)platinum(II)] tetrachloride, diarizidinylspermine, arsenic trioxide, 1-(11-dodecylamino-10-hydroxyundecyl)-3,7-dimethylxanthine, zorubicin, idarubicin, daunorubicin, bisantrene, mitoxantrone, pirarubicin, pinafide, valrubicin, amrubicin, antineoplaston, 3′-deansino-3′-morpholino-13-deoxo-10-hydroxycarminomycin, annamycin, galarubicin, elinafide, MEN10755, 4-demethoxy-3-deamino-3-aziridinyl-4-methylsulphonyl-daunombicin (see WO 00/50032), methoxtrexate, gemcitabine, and mixture thereof.

An example of a hypoxia activatable compound is tirapazamine.

Examples of proteasome inhibitors include, but are not limited to, lactacystin and bortezomib.

Examples of microtubule inhibitors/microtubule-stabilising agents include paclitaxel, vindesine sulfate, 3′,4′-didehydro-4′-deoxy-8′-norvincaleukoblastine, docetaxel, rhizoxin, dolastatin, mivobulin isethionate, auristatin, cemadotin, RPR109881, BMS184476, vinflunine, cryptophycin, 2,3,4,5,6-pentafluoro-N-(3-fluoro-4-methoxyphenyl) benzene sulfonamide, anhydrovinblastine, N,N-dimethyl-L-valyl-L-valyl-N-methyl-L-valyl-L-prolyl-L-proline-t-butylamide, TDX258, the epothilones (see for example U.S. Pat. Nos. 6,284,781 and 6,288,237) and BMS188797.

Some examples of topoisomerase inhibitors are topotecan, hycaptamine, irinotecan, rubitecan, 6-ethoxypropionyl-3′,4′-O-exo-benzylidene-chartreusin, 9-methoxy-N,N-dimethyl-5-nitropyrazolo[3,4,5-kl]acridine-2-(6H) propanamine, 1-amino-9-ethyl-5-fluoro-2,3-dihydro-9-hydroxy-4-methyl-1H,12H-benzo[de]pyrano[3′,4′:b,7]-indolizino[1,2b]quinoline-10,13(9H,15H)dione, lurtotecan, 7-[2-(N-isopropylamino) ethyl]-(20S)camptothecin, BNP1350, BNPI1100, BN80915, BN80942, etoposide phosphate, teniposide, sobuzoxane, 2′-dimethylamino-2′-deoxy-etoposide, GL331, N-[2-(dimethylamino)ethyl]-9-hydroxy-5,6-dimethyl-6H-pyrido[4,3-b]carbazole-1-carboxamide, asulacrine, (5a, 5aB, 8aa,9b)-9-[2-[N-[2-(dimethylamino)ethyl]-N-methylamino]ethyl]-5-[4-hydroxy-3,5-dimethoxyphenyl]-5,5a,6,8,8a,9-hexohydrofuro (3′,4′:6,7)naphtho(2,3-d)-1,3-dioxol-6-one, 2,3-(methylenedioxy)-5-methyl-7-hydroxy-8-methoxybenzo[c]-phenanthridinium, 6,9-bis[(2-aminoethyl)amino] benzo[g]isoguinoline-5,10-dione, 5-(3-aminopropylamino)-7,10-dihydroxy-2-(2-hydroxyethylaminomethyl)-6H-pyrazolo[4,5,1 -de]acridin-6-one, N-[1-[2-(diethylamino)ethylamino]-7-methoxy-9-oxo-9H-thioxanthen-4-ylmethyl]formamide,N-(2-(dimethylamino)ethyl)acridine-4-carboxamide, 6-[[2-(dimethylamino)ethyl]amino]-3-hydroxy-7H-indeno[2,1 -c]quinolin-7-one, dimesna, and camptostar.

Other useful anti-cancer agents that can be used in combination with the present compounds include thymidilate synthase inhibitors, such as 5-fluorouracil.

In one embodiment, inhibitors of mitotic kinesins include, but are not limited to, inhibitors of KSP, inhibitors of MKLP1, inhibitors of CENP-E, inhibitors of MCAK, inhibitors of Kif14, inhibitors of Mphosph1 and inhibitors of Rab6-KIFL.

The phrase “inhibitors of kinases involved in mitotic progression” include, but are not limited to, inhibitors of aurora kinase, inhibitors of Polo-like kinases (PLK) (in particular inhibitors of PLK-1), inhibitors of bub-1 and inhibitors of bub-R1.

The phrase “antiproliferative agents” includes antisense RNA and DNA oligonucleotides such as G3139, ODN698, RVASKRAS, GEM231, and INX3001, and antimetabolites such as enocitabine, carmofur, tegafur, pentostatin, doxifluridine, trimetrexate, fludarabine, capecitabine, galocitabine, cytarabine ocfosfate, fosteabine sodium hydrate, raltitrexed, paltitrexid, emitefur, tiazofurin, decitabine, nolatrexed, pemetrexed, nelzarabine, 2′-deoxy-2′-methylidenecytidine, 2′-fluoromethylene-2′-deoxycytidine, N-[5-(2,3-dihydro-benzofuryl)sulfonyl]-N′-(3,4-dichlorophenyl)urea, N6-[4-deoxy-4-[N2-[2(E),4(E)-tetradecadienoyl]glycylamino]-L-glycero-B-L-manno-heptopyranosyl]adenine, aplidine, ecteinascidin, troxacitabine, 4-[2-amino-4-oxo-4,6,7,8-tetrahydro-3H-pyrimidino[5,4-b][1,4]thiazin-6-yl-(S)-ethyl]-2,5-thienoyl-L-glutamic acid, aminopterin, 5-flurouracil, alanosine, 11 -acetyl-8-(carbamoyloxymethyl)-4-formyl-6-methoxy-14-oxa-1,11 -diazatetracyclo(7.4.1.0.0)-tetradeca-2,4,6-trien-9-yl acetic acid ester, swainsonine, lometrexol, dexrazoxane, methioninase, 2′-cyano-2′-deoxy-N4-palmitoyl-1-B-D-arabino furanosyl cytosine and 3-aminopyridine-2-carboxaldehyde thiosemicarbazone.

Examples of monoclonal antibody targeted therapeutic agents include those therapeutic agents which have cytotoxic agents or radioisotopes attached to a cancer cell specific or target cell specific monoclonal antibody. Examples include Bexxar.

Examples of monoclonal antibody therapeutics useful for treating cancer include Erbitux (Cetuximab).

The phrase “HMG-CoA reductase inhibitors” refers to inhibitors of 3-hydroxy-3-methylglutaryl-CoA reductase. Examples of HMG-CoA reductase inhibitors that may be used include but are not limited to lovastatin (MEVACOR®; see U.S. Pat. Nos. 4,231,938, 4,294,926 and 4,319,039), simvastatin(ZOCOR®; see U.S. Pat. Nos. 4,444,784, 4,820,850 and 4,916,239), pravastatin (PRAVACHOL®; see U.S. Pat. Nos. 4,346,227, 4,537,859, 4,410,629, 5,030,447 and 5,180,589), fluvastatin (LESCOL®; see U.S. Pat. Nos. 5,354,772, 4,911,165, 4,929,437, 5,189,164, 5,118,853, 5,290,946 and 5,356,896) and atorvastatin (LIPITOR®; see U.S. Pat. Nos. 5,273,995, 4,681,893, 5,489,691 and 5,342,952). The structural formulas of these and additional HMG-CoA reductase inhibitors that may be used in the instant methods are described at page 87 of M. Yalpani, “Cholesterol Lowering Drugs”, Chemistry & Industry, pp. 85-89 (5 Feb. 1996) and U.S. Pat. Nos. 4,782,084 and 4,885,314. The term HMG-CoA reductase inhibitor as used herein includes all pharmaceutically acceptable lactone and open-acid forms (i.e., where the lactone ring is opened to form the free acid) as well as salt and ester forms of compounds which have HMG-CoA reductase inhibitory activity, and therefore the use of such salts, esters, open acid and lactone forms is included in the scope of this invention.

The phrase “prenyl-protein transferase inhibitor” refers to a compound which inhibits any one or any combination of the prenyl-protein transferase enzymes, including farnesyl-protein transferase (FPTase), geranylgeranyl-protein transferase type I (GGPTase-I), and geranylgeranyl-protein transferase type-II (GGPTase-II, also called Rab GGPTase).

Examples of prenyl-protein transferase inhibitors can be found in the following publications and patents: WO 96/30343, WO 97/18813, WO 97/21701, WO 97/23478, WO 97/38665, WO 98/28980, WO 98/29119, WO 95/32987, U.S. Pat. Nos. 5,420,245, 5,523,430, 5,532,359, 5,510,510, 5,589,485, 5,602,098, European Patent Publ. 0 618 221, European Patent Publ. 0 675 112, European Patent Publ. 0 604181, European Patent Publ. 0 696 593, WO 94/19357, WO 95/08542, WO 95/11917, WO 95/12612, WO 95/12572, WO 95/10514, U.S. Pat. No. 5,661,152, WO 95/10515, WO 95/10516, WO 95/24612, WO 95/34535, WO 95/25086, WO 96/05529, WO 96/06138, WO 96/06193, WO 96/16443, WO 96/21701, WO 96/21456, WO 96/22278, WO 96/24611, WO 96/24612, WO 96/05168, WO 96/05169, WO 96/00736, U.S. Pat. No. 5,571,792, WO 96/17861, WO 96/33159, WO 96/34850, WO 96/34851, WO 96/30017, WO 96/30018, WO 96/30362, WO 96/30363, WO 96/31111, WO 96/31477, WO 96/31478, WO 96/31501, WO 97/00252, WO 97/03047, WO 97/03050, WO 97/04785, WO 97/02920, WO 97/17070, WO 97/23478, WO 97/26246, WO, 97/30053, WO 97/44350, WO 98/02436, and U.S. Pat. No. 5,532,359. For an example of the role of a prenyl-protein transferase inhibitor on angiogenesis see European of Cancer, Vol. 35, No. 9, pp. 1394-1401(1999).

Examples of farnesyl protein transferase inhibitors include SARASAR™(4-[2-[4-[(11R)-3,10-dibromo-8-chloro-6,11-dihydro-5H-benzo[5,6]cyclohepta[1,2-b]pyridin-11-yl-]-1-piperidinyl]-2-oxoehtyl]-1-piperidinecarboxamide from Schering-Plough Corporation, Kenilworth, N.J.), tipifarnib (Zarnestra® or R115777 from Janssen Pharmaceuticals), L778,123 (a farnesyl protein transferase inhibitor from Merck & Company, Whitehouse Station, N.J.), BMS 214662 (a farnesyl protein transferase inhibitor from Bristol-Myers Squibb Pharmaceuticals, Princeton, N.J.).

The phrase “angiogenesis inhibitors” refers to compounds that inhibit the formation of new blood vessels, regardless of mechanism. Examples of angiogenesis inhibitors include, but are not limited to, tyrosine kinase inhibitors, such as inhibitors of the tyrosine kinase receptors Flt-1 (VEGFR1) and Flk-1/KDR (VEGFR2), inhibitors of epidermal-derived, fibroblast-derived, or platelet derived growth factors, MMP (matrix metalloprotease) inhibitors, integrin blockers, interferon-α (for example Intron and Peg-Intron), interleukin-12, pentosan polysulfate, cyclooxygenase inhibitors, including nonsteroidal anti-inflammatories (NSAIDs) like aspirin and ibuprofen as well as selective cyclooxygenase-2 inhibitors like celecoxib and rofecoxib (PNAS, Vol. 89, p. 7384 (1992); JNCI, Vol. 69, p. 475 (1982); Arch. Opthalmol., Vol. 108, p. 573 (1990); Anat. Rec., Vol. 238, p. 68 (1994); FEBS Letters, Vol. 372, p. 83 (1995); Clin. Orthop. Vol. 313, p. 76 (1995); J. Mol. Endocrinol., Vol. 16, p. 107 (1996); Jpn. J. Pharrnacol., Vol. 75, p. 105 (1997); Cancer Res., Vol. 57, p. 1625 (1997); Cell, Vol. 93, p. 705 (1998); Intl. J. Mol. Med., Vol. 2, p. 715 (1998); J. Biol. Chem., Vol. 274, p. 9116 (1999)), steroidal anti-inflammatories (such as corticosteroids, mineralocorticoids, dexamethasone, prednisone, prednisolone, methylpred, betamethasone), carboxyamidotriazole, combretastatin A-4, squalamine, 6-O-chloroacetyl-carbonyl)-fumagillol, thalidomide, angiostatin, troponin-1, angiotensin II antagonists (see Fernandez et al., J. Lab. Clin. Med. 105:141-145 (1985)), and antibodies to VEGF (see, Nature Biotechnology, Vol. 17, pp. 963-968 (October 1999); Kim et al., Nature, 362, 841-844 (1993); WO 00/44777; and WO 00/61186).

Other therapeutic agents that modulate or inhibit angiogenesis and may also be used in combination with the compounds of the instant invention include agents that modulate or inhibit the coagulation and fibrinolysis systems (see review in Clin. Chem. La. Med. 38:679-692 (2000)). Examples of such agents that modulate or inhibit the coagulation and fibrinolysis pathways include, but are not limited to, heparin (see Thromb. Haemost. 80:10-23 (1998)), low molecular weight heparins and carboxypeptidase U inhibitors (also known as inhibitors of active thrombin activatable fibrinolysis inhibitor [TAFIa]) (see Thrombosis Res. 101:329-354 (2001)). Examples of TAFIa inhibitors have been described in PCT Publication WO 03/013,526.

The phrase “agents that interfere with cell cycle checkpoints” refers to compounds that inhibit protein kinases that transduce cell cycle checkpoint signals, thereby sensitizing the cancer cell to DNA damaging agents. Such agents include inhibitors of ATR, ATM, the Chk1 and Chk2 kinases and cdk and cdc kinase inhibitors and are specifically exemplified by 7-hydroxystaurosporin, flavopiridol, CYC202 (Cyclacel) and BMS-387032.

The phrase “inhibitors of cell proliferation and survival signaling pathway” refers to agents that inhibit cell surface receptors and signal transduction cascades downstream of those surface receptors. Such agents include inhibitors of EGFR (for example gefitinib and erlotinib), antibodies to EGFR (for example C225), inhibitors of ERB-2 (for example trastuzumab), inhibitors of IGFR, inhibitors of cytokine receptors, inhibitors of MET, inhibitors of P13K (for example LY294002), serine/threonine kinases (including but not limited to inhibitors of Akt such as described in WO 02/083064, WO 02/083139, WO 02/083140 and WO 02/083138), inhibitors of Raf kinase (for example BAY-43-9006), inhibitors of MEEK (for example CI-1040 and PD-098059), inhibitors of mTOR (for example Wyeth CCI-779), and inhibitors of C-abl kinase (for example GLEEVEC™, Novartis Pharmaceuticals). Such agents include small molecule inhibitor compounds and antibody antagonists.

The phrase “apoptosis inducing agents” includes activators of TNF receptor family members (including the TRAIL receptors).

The invention also encompasses combinations with NSAID's which are selective COX-2 inhibitors. For purposes of this specification NSAID's which are selective inhibitors of COX-2 are defined as those which possess a specificity for inhibiting COX-2 over COX-1 of at least 100 fold as measured by the ratio of IC50 for COX-2 over IC50 for COX-1 evaluated by cell or microsomal assays. Inhibitors of COX-2 that are particularly useful in the instant method of treatment are: 3-phenyl-4-(4-(methylsulfonyl)phenyl)-2-(5H)-furanone; and 5-chloro-3-(4-methylsulfonyl)phenyl-2-(2-methyl-5 pyridinyl)pyridine; or a pharmaceutically acceptable salt thereof.

Compounds that have been described as specific inhibitors of COX-2 and are therefore useful in the present invention include, but are not limited to, parecoxib, CELIEBREX® and BEXTRA® or a pharmaceutically acceptable salt thereof.

Other examples of angiogenesis inhibitors include, but are not limited to, endostatin, ukrain, ranpirnase, IM862, 5-methoxy-4-[2-methyl-3-(3-methyl-2-butenyl)oxiranyl]-1-oxaspiro[2,5]oct-6-yl(chloroacetyl)carbamate, acetyldinanaline, 5-amino-1-[[3,5-dichloro-4-(4-chlorobenzoyl)phenyl]methyl]-1H-1,2,3-triazole-4-carboxamide, CM101, squalamine, combretastatin, RPI4610, NX31838, sulfated mannopentaose phosphate, 7,7-(carbonyl-bis[imino-N-methyl-4,2-pyrrolocarbonylimino[N-methyl-4,2-pyrrole]-carbonylimino]-bis-(1,3-naphthalene disulfonate), and 3-[(2,4-dimethylpyrrol-5-yl)methylene]-2-indolinone (SU5416).

As used above, “integrin blockers” refers to compounds which selectively antagonize, inhibit or counteract binding of a physiological ligand to the αᵥβ₃ integrin, to compounds which selectively antagonize, inhibit or counteract binding of a physiological ligand to the αᵥβ₅ integrin, to compounds which antagonize, inhibit or counteract binding of a physiological ligand to both the αᵥβ₃ integrin and the αᵥβ₅ integrin, and to compounds which antagonize, inhibit or counteract the activity of the particular integrin(s) expressed on capillary endothelial cells. The term also refers to antagonists of the αᵥβ₆, αᵥβ₈, α₁β₁, α₂β₁, α₅β₁, α₆β₁ and α₆β₄ integrins. The term also refers to antagonists of any combination of αᵥβ₃, αᵥβ₅, αᵥβ₆, αᵥβ₈, α₁β₁, α₂β₁, α₅β₁, α₆β₁ and α₆β₄ integrins.

Some examples of tyrosine kinase inhibitors include N-(trifluoromethylphenyl)-5-methylisoxazol-4-carboxamide, 3-[(2,4-dimethylpyrrol-5-yl)methylidenyl)indolin-2-one,17-(allylamino)-17-demethoxygeldanamycin, 4-(3-chloro-4-fluorophenylamino)-7-methoxy-6-[3-(4-morpholinyl)propoxyl]quinazoline, N-(3-ethynylphenyl)-6,7-bis(2-methoxyethoxy)-4-quinazolinamine, BIBX1382, 2,3,9,10,11,12-hexahydro-10-(hydroxymethyl)-10-hydroxy-9-methyl-9,12-epoxy-1H-diindolo[1,2,3-fg:3′,2′,1′-kl]pyrrolo[3,4-i][1,6]benzodiazocin-1-one, SH268, genistein, STI571, CEP2563, 4-(3-chlorophenylamino)-5,6-dimethyl-7H-pyrrolo[2,3-d]pyrimidinemethane sulfonate, 4-(3-bromo-4-hydroxyphenyl)amino-6,7-dimethoxyquinazoline, 4-(4′-hydroxyphenyl)amino-6,7-dimethoxyquinazoline, SU6668, STI571A, N-4-chlorophenyl-4-(4-pyridylmethyl)-1-phthalazinamine, and EMD121974.

Combinations with compounds other than anti-cancer compounds are also encompassed in the instant methods. For example, combinations of the present compounds with PPAR-γ (i.e., PPAR-gamma) agonists and PPAR-δ (i.e., PPAR-delta) agonists are useful in the treatment of certain malingnancies. PPAR-γ and PPAR-δ are the nuclear peroxisome proliferator-activated receptors γ and δ. The expression of PPAR-γ on endothelial cells and its involvement in angiogenesis has been reported in the literature (see J. Cardiovasc. Pharmacol. 1998; 31:909-913; J. Biol. Chem. 1999;274:9116-9121; Invest. Ophthalmol Vis. Sci. 2000; 41:2309-2317). More recently, PPAR-γ agonists have been shown to inhibit the angiogenic response to VEGF in vitro; both troglitazone and rosiglitazone maleate inhibit the development of retinal neovascularization in mice (Arch. Ophthamol. 2001; 119:709-717). Examples of PPAR-γ agonists and PPAR-γ/α agonists include, but are not limited to, thiazolidinediones (such as DRF2725, CS-011, troglitazone, rosiglitazone, and pioglitazone), fenofibrate, gemfibrozil, clofibrate, GW2570, SB219994, AR-H039242, JTT-501, MCC-555, GW2331, GW409544, NN2344, KRP297, NP0110, DRF4158, NN622, G1262570, PNU182716, DRF552926, 2-[(5,7-dipropyl-3-trifluoromethyl-1,2-benzisoxazol-6-yl)oxy]-2-methylpropionic acid, and 2(R)-7-(3-(2-chloro-4-(4-fluorophenoxy) phenoxy)propoxy)-2-ethylchromane-2-carboxylic acid.

In one embodiment, useful anti-cancer (also known as anti-neoplastic) agents that can be used in combination with the present compounds include, but are not limited, to Uracil mustard, Chlormethine, Ifosfamide, Melphalan, Chlorambucil, Pipobroman, Triethylenemelamine, Triethylenethiophosphoramine, Busulfan, Carmustine, Lomustine, Streptozocin, Dacarbazine, Floxuridine, Cytarabine, 6-Mercaptopurine, 6-Thioguanine, Fludarabine phosphate, oxaliplatin, leucovirin, oxaliplatin (ELOXATIN™ from Sanofi-Synthelabo Pharmaeuticals, France), Pentostatine, Vinblastine, Vincristine, Vindesine, Bleomycin, Dactinomycin, Daunorubicin, Doxorubicin, Epirubicin, Idarubicin, Mithramycin, Deoxycoformycin, Mitomycin-C, L-Asparaginase, Teniposide 17α-Ethinylestradiol, Diethylstilbestrol, Testosterone, Prednisone, Fluoxymesterone, Dromostanolone propionate, Testolactone, Megestrolacetate, Methylprednisolone, Methyltestosterone, Prednisolone, Triamcinolone, Chlorotrianisene, Hydroxyprogesterone, Aminoglutethimide, Estramustine, Medroxyprogesteroneacetate, Leuprolide, Flutamide, Toremifene, goserelin, Cisplatin, Carboplatin, Hydroxyurea, Amsacrine, Procarbazine, Mitotane, Mitoxantrone, Levamisole, Navelbene, Anastrazole, Letrazole, Capecitabine, Reloxafine, Droloxafine, Hexamethylmelamine, doxorubicin (adriamycin), cyclophosphamide (cytoxan), gemcitabine, interferons, pegylated interferons, Erbitux and mixtures thereof.

Another embodiment of the present invention is the use of the present compounds in combination with gene therapy for the treatment of cancer. For an overview of genetic strategies to treating cancer, see Hall et al (Am J Hum Genet 61:785-789, 1997) and Kufe et al (Cancer Medicine, 5th Ed, pp 876-889, BC Decker, Hamilton 2000). Gene therapy can be used to deliver any tumor suppressing gene. Examples of such genes include, but are not limited to, p53, which can be delivered via recombinant virus-mediated gene transfer (see U.S. Pat. No. 6,069,134, for example), a uPA/uPAR antagonist (“Adenovirus-Mediated Delivery of a uPA/uPAR Antagonist Suppresses Angiogenesis-Dependent Tumor Growth and Dissemination in Mice,” Gene Therapy, August 1998;5(8):1105-13), and interferon gamma (J Immunol 2000; 1 64:217-222).

The present compounds can also be administered in combination with one or more inhibitor of inherent multidrug resistance (MDR), in particular MDR associated with high levels of expression of transporter proteins. Such MDR inhibitors include inhibitors of p-glycoprotein (P-gp), such as LY335979, XR9576, OC144-093, R101922, VX853 and PSC833 (valspodar).

The present compounds can also be employed in conjunction with one or more anti-emetic agents to treat nausea or emesis, including acute, delayed, late-phase, and anticipatory emesis, which may result from the use of a compound of the present invention, alone or with radiation therapy. For the prevention or treatment of emesis, a compound of the present invention may be used in conjunction with one or more other anti-emetic agents, especially neurokinin-1 receptor antagonists, 5HT3 receptor, antagonists, such as ondansetron, granisetron, tropisetron, and zatisetron, GABAB receptor agonists, such as baclofen, a corticosteroid such as Decadron (dexamethasone), Kenalog, Aristocort, Nasalide, Preferid, Benecorten or those as described in U.S. Pat. Nos. 2,789,118, 2,990,401, 3,048,581, 3,126,375, 3,929,768, 3,996,359, 3,928,326 and 3,749,712, an antidopaminergic, such as the phenothiazines (for example prochlorperazine, fluphenazine, thioridazine and mesoridazine), metoclopramide or dronabinol. In one embodiment, an anti-emesis agent selected from a neurokinin-1 receptor antagonist, a 5HT3 receptor antagonist and a corticosteroid is administered as an adjuvant for the treatment or prevention of emesis that may result upon administration of the present compounds.

Examples of neurokinin-1 receptor antagonists that can be used in conjunction with the present compounds are described in U.S. Pat. Nos. 5,162,339, 5,232,929, 5,242,930, 5,373,003, 5,387,595, 5,459,270, 5,494,926, 5,496,833, 5,637,699, and 5,719,147, content of which are incorporated herein by reference. In an embodiment, the neurokinin-1 receptor antagonist for use in conjunction with the compounds of the present invention is selected from: 2-(R)-(1-(R)-(3,5-bis(trifluoromethyl)phenyl)ethoxy)-3-(S)-(4-fluorophenyl)-4-(3-(5-oxo-1H,4H-1,2,4-triazolo)methyl)morpholine, or a pharmaceutically acceptable salt thereof, which is described in U.S. Pat. No. 5,719,147.

A compound of the present invention may also be administered with one or more immunologic-enhancing drug, such as for example, levamisole, isoprinosine and Zadaxin.

Thus, the present invention encompasses the use of the present compounds (for example, for treating or preventing cellular proliferative diseases) in combination with a second compound selected from: an estrogen receptor modulator, an androgen receptor modulator, retinoid receptor modulator, a cytotoxic/cytostatic agent, an antiproliferative agent, a prenyl-protein transferase inhibitor, an HMG-CoA reductase inhibitor, an angiogenesis inhibitor, a PPAR-γ agonist, a PPAR-δ agonist, an inhibitor of inherent multidrug resistance, an anti-emetic agent, an immunologic-enhancing drug, an inhibitor of cell proliferation and survival signaling, an agent that interfers with a cell cycle checkpoint, and an apoptosis inducing agent.

In one embodiment, the present invention empassesses the composition and use of the present compounds in combination with a second compound selected from: a cytostatic agent, a cytotoxic agent, taxanes, a topoisomerase II inhibitor, a topoisomerase I inhibitor, a tubulin interacting agent, hormonal agent, a thymidilate synthase inhibitors, anti-metabolites, an alkylating agent, a farnesyl protein transferase inhibitor, a signal transduction inhibitor, an EGFR kinase inhibitor, an antibody to EGFR, a C-abl kinase inhibitor, hormonal therapy combinations, and aromatase combinations.

The term “treating cancer” or “treatment of cancer” refers to administration to a mammal afflicted with a cancerous condition and refers to an effect that alleviates the cancerous condition by killing the cancerous cells, but also to an effect that results in the inhibition of growth and/or metastasis of the cancer.

In one embodiment, the angiogenesis inhibitor to be used as the second compound is selected from a tyrosine kinase inhibitor, an inhibitor of epidermal-derived growth factor, an inhibitor of fibroblast-derived growth factor, an inhibitor of platelet derived growth factor, an MW (matrix metalloprotease) inhibitor, an integrin blocker, interferon-α, interleukin-12, pentosan polysulfate, a cyclooxygenase inhibitor, carboxyamidotriazole, combretastatin A-4, squalamine, 6-(O-chloroacetylcarbonyl)-fumagillol, thalidomide, angiostatin, troponin-1, or an antibody to VEGF. In an embodiment, the estrogen receptor modulator is tamoxifen or raloxifene.

Also included in the present invention is a method of treating cancer comprising administering a therapeutically effective amount of at least one compound of the present invention in combination with radiation therapy and at least one compound selected from: an estrogen receptor modulator, an androgen receptor modulator, retinoid receptor modulator, a cytotoxic/cytostatic agent, an antiproliferative agent, a prenyl-protein transferase inhibitor, an HMG-CoA reductase inhibitor, an angiogenesis inhibitor, a PPAR-γ agonist, a PPAR-δ agonist, an inhibitor of inherent multidrug resistance, an anti-emetic agent, an immunologic-enhancing drag, an inhibitor of cell proliferation and survival signaling, an agent that interfers with a cell cycle checkpoint, and an apoptosis inducing agent.

Yet another embodiment of the invention is a method of treating cancer comprising administering a therapeutically effective amount of at least one compound of the present invention in combination with paclitaxel or trastuzumab.

The present invention also includes a pharmaceutical composition useful for treating or preventing the various disease states mentioned herein cellular proliferation diseases (such as cancer, hyperplasia, cardiac hypertrophy, autoimmune diseases, fungal disorders, arthritis, graft rejection, inflammatory bowel disease, immune disorders, inflammation, and cellular proliferation induced after medical procedures) that comprises a therapeutically effective amount of at least one compound of the present invention and at least one compound selected from: an estrogen receptor modulator, an androgen receptor modulator, a retinoid receptor modulator, a cytotoxic/cytostatic agent, an antiproliferative agent, a prenyl-protein transferase inhibitor, an HMG-CoA reductase inhibitor, an angiogenesis inhibitor, a PPAR-γ agonist, a PPAR-δ agonist, an inhibitor of cell proliferation and survival signaling, an agent that interfers with a cell cycle checkpoint, and an apoptosis inducing agent.

When the disease being treated by the cathepsin inhibitor compounds of the present invention is inflammatory disease, an embodiment of the present invention comprises administering: (a) a therapeutically effective amount of at least one compound of the present cathepsin inhibitors (e.g., a compound according to Formula I-XXVI) or a pharmaceutically acceptable salt, solvate or ester thereof concurrently or sequentially with (b) at least one medicament selected from the group consisting of: disease modifying antirheumatic drugs; nonsteroidal anti-inflammatory drugs; COX-2 selective inhibitors; COX-1 inhibitors; immunosuppressives (non-limiting examples include methotrexate, cyclosporin, FK506); steroids; PDE IV inhibitors, anti-TNF-α compounds, TNF-alpha-convertase inhibitors, cytokine inhibitors, MMP inhibitors, glucocorticoids, chemokine inhibitors, CB2-selective inhibitors, p38 inhibitors, biological response modifiers; anti-inflammatory agents and therapeutics.

Another embodiment of the present invention is directed to a method of inhibiting or blocking T-cell mediated chemotaxis in a patient in need of such treatment the method comprising administering to the patient a therapeutically effective amount of at least one compound of the present cathepsin inhibitors (e.g., a compound according to formula I-XXVII) or a pharmaceutically acceptable salt, solvate or ester thereof.

Another embodiment of this invention is directed to a method of treating inflammatory bowel disease in a patient in need of such treatment comprising administering to the patient a therapeutically effective amount of at least one compound according to the present cathepsin inhibitors or a pharmaceutically acceptable salt, solvate or ester thereof.

Another embodiment of this invention is directed to a method of treating or preventing graft rejection in a patient in need of such treatment comprising administering to the patient a therapeutically effective amount of at least one compound according to the present cathepsin inhibitors, or a pharmaceutically acceptable salt, solvate or ester thereof.

Another embodiment of this invention is directed to a method comprising administering to the patient a therapeutically effective amount of: (a) at least one compound according to the present cathepsin inhibitors, or a pharmaceutically acceptable salt, solvate or ester thereof concurrently or sequentially with (b) at least one compound selected from the group consisting of: cyclosporine A, FK-506, FTY720, beta-Interferon, rapamycin, mycophenolate, prednisolone, azathioprine, cyclophosphamide and an antilymphocyte globulin.

Another embodiment of this invention is directed to a method of treating multiple sclerosis in a patient in need of such treatment the method comprising administering to the patient a therapeutically effective amount of: (a) at least one compound according to the present cathepsin inhibitors, or a pharmaceutically acceptable salt, solvate or ester thereof concurrently or sequentially with (b) at least one compound selected from the group consisting of: beta-interferon, glatiramer acetate, glucocorticoids, methotrexate, azothioprine, mitoxantrone, VLA-4 inhibitors and/or CB2-selective inhibitors.

Another embodiment of this invention is directed to a method of treating multiple sclerosis in a patient in need of such treatment the method comprising administering to the patient a therapeutically effective amount of: a) at least one compound according to the present cathepsin inhibitors, or a pharmaceutically acceptable salt, solvate or ester thereof concurrently or sequentially with (b) at least one compound selected from the group consisting of: methotrexate, cyclosporin, leflunimide, sulfasalazine, β-methasone, β-interferon, glatiramer acetate, prednisone, etonercept, and infliximab.

Another embodiment of this invention is directed to a method of treating rheumatoid arthritis in a patient in need of such treatment the method comprising administering to the patient a therapeutically effective amount of: (a) at least one compound according to the present cathepsin inhibitors or a pharmaceutically acceptable salt, solvate or ester thereof concurrently or sequentially with (b) at least one compound selected from the group consisting of: COX-2 inhibitors, COX inhibitors, immunosuppressives, steroids, PDE IV inhibitors, anti-TNF-α compounds, MMP inhibitors, glucocorticoids, chemokine inhibitors, CB2-selective inhibitors, caspase (ICE) inhibitors and other classes of compounds indicated for the treatment of rheumatoid arthritis.

Another embodiment of this invention is directed to a method of treating psoriasis in a patient in need of such treatment the method comprising administering to the patient a therapeutically effective amount of: a) at least one compound according to present cathepsin inhibitors, or a pharmaceutically acceptable salt, solvate or ester thereof concurrently or sequentially with (b) at least one compound selected from the group consisting of: immunosuppressives, steroids, and anti-TNF-α compounds.

Another embodiment of this invention is directed to a method of treating a disease selected from the group consisting of: inflammatory disease, rheumatoid arthritis, multiple sclerosis, inflammatory bowel disease, graft rejection, psoriasis, fixed drug eruptions, cutaneous delayed-type hypersensitivity responses, tuberculoid leprosy, type I diabetes, viral meningitis and tumors in a patient in need of such treatment; such method comprising administering to the patient an effective amount of at least one compound according to present cathepsin inhibitors, or a pharmaceutically acceptable salt, solvate or ester thereof.

Another embodiment of this invention is directed to a method of treating a disease selected from the group consisting of inflammatory disease, rheumatoid arthritis, multiple sclerosis, inflammatory bowel disease, graft rejection, psoriasis, fixed drug eruptions, cutaneous delayed-type hypersensitivity responses, tuberculoid leprosy and cancer in a patient in need of such treatment, such method comprising administering to the patient an effective amount of at least one compound according to the present cathepsin inhibitors, or a pharmaceutically acceptable salt, solvate or ester thereof.

Another embodiment of this invention is directed to a method of treating a disease selected from the group consisting of inflammatory disease, rheumatoid arthritis, multiple sclerosis, inflammatory bowel disease, graft rejection, psoriasis, fixed drug eruptions, cutaneous delayed-type hypersensitivity responses and tuberculoid leprosy, type I diabetes, viral meningitis and cancer in a patient in need of such treatment, such method comprising administering to the patient an effective amount of (a) at least one compound according to the present cathepsin inhibitors, or a pharmaceutically acceptable salt, solvate or ester thereof concurrently or sequentially with (b) at least one medicament selected from the group consisting of: disease modifying antirheumatic drugs; nonsteroidal anti-inflammatory drugs; COX-2 selective inhibitors; COX-1 inhibitors; immunosuppressives; steroids; PDE IV inhibitors, anti-TNF-α compounds, MMP inhibitors, glucocorticoids, chemokine inhibitors, CB2-selective inhibitors, biological response modifiers; anti-inflammatory agents and therapeutics.

When the present invention involves a method of treating a cardiovascular disease, in addition to administering the cathepsin inhibitors of the present invention, the method further comprises administering to the subject in need one or more pharmacological or therapeutic agents or drugs such as cholesterol biosynthesis inhibitors and/or lipid-lowering agents discussed below.

Non-limiting examples of cholesterol biosynthesis inhibitors for use in the compositions, therapeutic combinations and methods of the present invention include competitive inhibitors of HMG CoA reductase, the rate-limiting step in cholesterol biosynthesis, squalene synthase inhibitors, squalene epoxidase inhibitors and mixtures thereof. Non-limiting examples of suitable HMG CoA reductase inhibitors include statins such as lovastatin (for example MEVACOR® which is available from Merck & Co.), pravastatin (for example PRAVACHOL® which is available from Bristol Meyers Squibb), fluvastatin, simvastatin (for example ZOCOR® which is available from Merck & Co.), atorvastatin, cerivastatin, rosuvastatin, rivastatin (sodium 7-(4-fluorophenyl)-2,6-diisopropyl-5-methoxymethylpyridin-3-yl)-3,5-dihydroxy-6-heptanoate, CI-981 and pitavastatin (such as NK-104 of Negma Kowa of Japan); HMG CoA synthetase inhibitors, for example L-659,699 ((E,E)-11-[3′R-(hydroxy-methyl)-4′-oxo-2′R-oxetanyl]-3,5,7R-trimethyl-2,4-undecadienoic acid); squalene synthesis inhibitors, for example squalestatin 1; and squalene epoxidase inhibitors, for example, NB-598 ((E)-N-ethyl-N-(6,6-dimethyl-2-hepten-4-ynyl)-3-[(3,3′-bithiophen-5-yl)methoxy]benzene-methanamine hydrochloride) and other sterol biosynthesis inhibitors such as DMP-565. Preferred HMG CoA reductase inhibitors include lovastatin, pravastatin and simvastatin.

In another embodiment, the method of treatment comprises administering the present cathepsin inhibitors in combination with one or more cardiovascular agents and one or more cholesterol biosynthesis inhibitors. Preferably the cholesterol biosynthesis inhibitor comprises one or more HMG CoA reductase inhibitors, such as, for example, lovastatin, pravastatin and/or simvastatin.

In another alternative embodiment, the method treatment of the present invention can further comprise administering nicotinic acid (niacin) and/or derivatives thereof coadministered with or in combination with the cardiovascular agent(s) and sterol absorption inhibitor(s) discussed above.

As used herein, “nicotinic acid derivative” means a compound comprising a pyridine-3-carboxylate structure or a pyrazine-2-carboxylate structure, including acid forms, salts, esters, zwitterions and tautomers, where available. Examples of nicotinic acid derivatives include niceritrol, nicofuranose and acipimox (5-methyl pyrazine-2-carboxylic acid 4-oxide). Nicotinic acid and its derivatives inhibit hepatic production of VLDL and its metabolite LDL and increases HDL and apo A-1 levels. An example of a suitable nicotinic acid product is NIASPAN® (niacin extended-release tablets) which are available from Kos.

In another alternative embodiment, the method of treatment of the present invention can further comprise administering one or more AcylCoA:Cholesterol O-acyltransferase (“ACAT”) Inhibitors, which can reduce LDL and VLDL levels, coadministered with or in combination with the cardiovascular agent(s) and sterol absorption inhibitor(s) discussed above. ACAT is an enzyme responsible for esterifying excess intracellular cholesterol and may reduce the synthesis of VLDL, which is a product of cholesterol esterification, and overproduction of apo B-100-containing lipoproteins.

Non-limiting examples of useful ACAT inhibitors include avasimibe ([[2,4,6-tris(1-methylethyl)phenyl]acetyl]sulfamic acid, 2,6-bis(1-methylethyl)phenyl ester, formerly known as CI-1011), HL-004, lecimibide (DuP-128) and CL-277082 (N-(2,4-difluorophenyl)-N-[[4-(2,2-dimethylpropyl)phenyl]methyl]-N-heptylurea). See P. Chang et al., “Current, New and Future Treatments in Dyslipidaemia and Atherosclerosis”, Drugs July2000;60(1); 55-93, which is incorporated by reference herein.

In another alternative embodiment, the method of treatment of the present invention can further comprise administering probucol or derivatives thereof (such as AGI-1067 and other derivatives disclosed in U.S. Pat. Nos. 6,121,319 and 6,147,250), which can reduce LDL levels, coadministered with or in combination with the cardiovascular agent(s) and sterol absorption inhibitor(s) discussed above.

In another alternative embodiment, the method of treatment of the present invention can further comprise administering fish oil, which contains Omega 3 fatty acids (3-PUFA), which can reduce VLDL and triglyceride levels, coadministered with or in combination with the cardiovascular agent(s) and sterol absorption inhibitor(s) discussed above. Generally, a total daily dosage of fish oil or Omega 3 fatty acids can range from about 1 to about 30 grams per day in single or 2-4 divided doses.

In another alternative embodiment, the method of treatment of the present invention can further comprise administering natural water soluble fibers, such as psyllium, guar, oat and pectin, which can reduce cholesterol levels, coadministered with or in combination with the cardiovascular agent(s) and sterol absorption inhibitor(s) discussed above. Generally, a total daily dosage of natural water soluble fibers can range from about 0.1 to about 10 grams per day in single or 2-4 divided doses.

In another alternative embodiment, the method of treatment of the present invention can further comprise administering plant sterols, plant stanols and/or fatty acid esters of plant stanols, such as sitostanol ester used in BENECOL® margarine, which can reduce cholesterol levels, coadministered with or in combination with the cardiovascular agent(s) and sterol absorption inhibitor(s) discussed above. Generally, a total daily dosage of plant sterols, plant stanols and/or fatty acid esters of plant stanols can range from about 0.5 to about 20 grams per day in single or 2-4 divided doses.

In another alternative embodiment, the method of treatment of the present invention can further comprise administering antioxidants, such as probucol, tocopherol, ascorbic acid, β-carotene and selenium, or vitamins such as vitamin B₆ or vitamin B₁₂, coadministered with or in combination with the cardiovascular agent(s) and sterol absorption inhibitor(s) discussed above. Generally, a total daily dosage of antioxidants or vitamins can range from about 0.05 to about 10 grams per day in single or 2-4 divided doses.

In another alternative embodiment, the method of treatment of the present invention can further comprise administering one or more bile acid sequestrants (insoluble anion exchange resins), coadministered with or in combination with the cardiovascular agents and sterol absorption inhibitor(s) discussed above.

Bile acid sequestrants bind bile acids in the intestine, interrupting the enterohepatic circulation of bile acids and causing an increase in the faecal excretion of steroids. Use of bile acid sequestrants is desirable because of their non-systemic mode of action. Bile acid sequestrants can lower intrahepatic cholesterol and promote the synthesis of apo B/E (LDL) receptors which bind LDL from plasma to further reduce cholesterol levels in the blood.

Non-limiting examples of suitable bile acid sequestrants include cholestyramine (a styrene-divinylbenzene copolymer containing quaternary ammonium cationic groups capable of binding bile acids, such as QUESTRAN® or QUESTRAN LIGHT® cholestyramine which are available from Bristol-Myers Squibb), colestipol (a copolymer of diethylenetriamine and 1-chloro-2,3-epoxypropane, such as COLESTID® tablets which are available from Pharmacia), colesevelam hydrochloride (such as WelChol® Tablets (poly(allylamine hydrochloride) cross-linked with epichlorohydrin and alkylated with 1-bromodecane and (6-bromohexyl)-trimethylammonium bromide) which are available from Sankyo), water soluble derivatives such as 3,3-ioene, N-(cycloalkyl) alkylamines and poliglusam, insoluble quaternized polystyrenes, saponins and mixtures thereof. Other useful bile acid sequestrants are disclosed in PCT Patent Applications Nos. WO 97/11345 and WO 98/57652, and U.S. Pat. Nos. 3,692,895 and 5,703,188 which are incorporated herein by reference. Suitable inorganic cholesterol sequestrants include bismuth salicylate plus montmorillonite clay, aluminum hydroxide and calcium carbonate antacids.

Also useful with the present invention are methods of treatment that can further comprise administering at least one (one or more) activators for peroxisome proliferator-activated receptors (PPAR). These activators act as agonists for the peroxisome proliferator-activated receptors. Three subtypes of PPAR have been identified, and these are designated as peroxisome proliferator-activated receptor alpha (PPARα), peroxisome proliferator-activated receptor gamma (PPARγ) and peroxisome proliferator-activated receptor delta (PPARδ). It should be noted that PPARδ is also referred to in the literature as PPARβ and as NUC1, and each of these names refers to the same receptor.

PPARα regulates the metabolism of lipids. PPARα is activated by fibrates and a number of medium and long-chain fatty acids, and it is involved in stimulating β-oxidation of fatty acids. The PPARγ receptor subtypes are involved in activating the program of adipocyte differentiation and are not involved in stimulating peroxisome proliferation in the liver. PPARδ has been identified as being useful in increasing high density lipoprotein (HDL) levels in humans. See, e.g., WO 97/28149.

PPARα activator compounds are useful for, among other things, lowering triglycerides, moderately lowering LDL levels and increasing HDL levels. Useful examples of PPARα activators include the fibrates discussed above.

Other examples of PPARα activators useful with the practice of the present invention include suitable fluorophenyl compounds as disclosed in U.S. Pat. No. 6,028,109 which is incorporated herein by reference; certain substituted phenylpropionic compounds as disclosed in WO 00/75103 which is incorporated herein by reference; and PPARα activator compounds as disclosed in WO 98/43081 which is incorporated herein by reference.

Non-limiting examples of PPARγ activator include suitable derivatives of glitazones or thiazolidinediones, such as, troglitazone (such as REZULIN® troglitazone (-5-[[4-[3,4-dihydro-6-hydroxy-2,5,7,8-tetramethyl-2H-1-benzopyran-2-yl)methoxy]phenyl]methyl]-2,4-thiazolidinedione) commercially available from Parke-Davis); rosiglitazone (such as AVANDIA® rosiglitazone maleate (-5-[[4-[2-(methyl-2-pyridinylamino)ethoxy]phenyl]methyl]-2,4-thiazolidinedione, (Z) -2-butenedioate) (1:1) commercially available from SmithKline Beecham) and pioglitazone (such as ACTOS™ pioglitazone hydrochloride (5-[[4-[2-(5-ethyl-2-pyridinyl)ethoxy]phenyl]methyl]-2,4-] thiazolidinedione monohydrochloride) commercially available from Takeda Pharmaceuticals). Other useful thiazolidinediones include ciglitazone, englitazone, darglitazone and BRL 49653 as disclosed in WO 98/05331 which is incorporated herein by reference; PPARγ activator compounds disclosed in WO 00/76488 which is incorporated herein by reference; and PPARγ activator compounds disclosed in U.S. Pat. No. 5,994,554 which is incorporated herein by reference.

Other useful classes of PPARγ activator compounds include certain acetylphenols as disclosed in U.S. Pat. No. 5,859,051 which is incorporated herein by reference; certain quinoline phenyl compounds as disclosed in WO 99/20275 which is incorporated herein by reference; aryl compounds as disclosed by WO 99/38845 which is incorporated herein by reference; certain 1,4-disubstituted phenyl compounds as disclosed in WO 00/63161; certain aryl compounds as disclosed in WO 01/00579 which is incorporated herein by reference; benzoic acid compounds as disclosed in WO 01/12612 & WO 01/12187 which are incorporated herein by reference; and substituted 4-hydroxy-phenylalconic acid compounds as disclosed in WO 97/31907 which is incorporated herein by reference.

PPARδ compounds are useful for, among other things, lowering triglyceride levels or raising HDL levels. Non-limiting examples of PPARδ activators include suitable thiazole and oxazole derivates, such as C.A.S. Registry No. 317318-32-4, as disclosed in WO 01/00603 which is incorporated herein by reference); certain fluoro, chloro or thio phenoxy phenylacetic acids as disclosed in WO 97/28149 which is incorporated herein by reference; suitable non-β-oxidizable fatty acid analogues as disclosed in U.S. Pat. No. 5,093,365 which is incorporated herein by reference; and PPARδ compounds as disclosed in WO 99/04815 which is incorporated herein by reference.

Moreover, compounds that have multiple functionality for activating various combinations of PPARα, PPARγ and PPARδ are also useful with the practice of the present invention. Non-limiting examples include certain substituted aryl compounds as disclosed in U.S. Pat. No. 6,248,781; WO 00/23416; WO 00/23415; WO 00/23425; WO 00/23445; WO 00/23451; and WO 00/63153, all of which are incorporated herein by reference, are described as being useful PPARα and/or PPARγ activator compounds. Other non-limiting examples of useful PPARα and/or PPARγ activator compounds include activator compounds as disclosed in WO 97/25042 which is incorporated herein by reference; activator compounds as disclosed in WO 00/63190 which is incorporated herein by reference; activator compounds as disclosed in WO 01/21181 which is incorporated herein by reference; biaryl-oxa(thia)zole compounds as disclosed in WO 01/16120 which is incorporated herein by reference; compounds as disclosed in WO 00/63196 and WO 00/63209 which are incorporated herein by reference; substituted 5-aryl-2,4-thiazolidinediones compounds as disclosed in U.S. Pat. No. 6,008,237 which is incorporated herein by reference; arylthiazolidinedione and aryloxazolidinedione compounds as disclosed in WO 00/78312 and WO 00/78313G which are incorporated herein by reference; GW2331 or (2-(4-[difluorophenyl]-1heptylureido)ethyl]phenoxy)-2-methylbutyric compounds as disclosed in WO 98/05331 which is incorporated herein by reference; aryl compounds as disclosed in U.S. Pat. No. 6,166,049 which is incorporated herein by reference; oxazole compounds as disclosed in WO 01/17994 which is incorporated herein by reference; and dithiolane compounds as disclosed in WO 01/25225 and WO 01/25226 which are incorporated herein by reference.

Other useful PPAR activator compounds include substituted benzylthiazolidine-2,4-dione compounds as disclosed in WO 01/14349, WO 01/14350 and WO/01/04351 which are incorporated herein by reference; mercaptocarboxylic compounds as disclosed in WO 00/50392 which is incorporated herein by reference; ascofuranone compounds as disclosed in WO 00/53563 which is incorporated herein by reference; carboxylic compounds as disclosed in WO 99/46232 which is incorporated herein by reference; compounds as disclosed in WO 99/12534 which is incorporated herein by reference; benzene compounds as disclosed in WO 99/15520 which is incorporated herein by reference; o-anisamide compounds as disclosed in WO 01/21578 which is incorporated herein by reference; and PPAR activator compounds as disclosed in WO 01/40192 which is incorporated herein by reference.

Also useful with the present invention are methods of treatment which further comprise administering hormone replacement agents and compositions. Useful hormone agents and compositions for hormone replacement therapy of the present invention include androgens, estrogens, progestins, their pharmaceutically acceptable salts and derivatives. Combinations of these agents and compositions are also useful.

The cathepsin inhibitors of the present invention are useful in the treatment of central nervous system diseases such as depression, cognitive function diseases and neurodegenerative diseases such as Parkinson's disease, senile dementia as in Alzheimer's disease, and psychoses of organic origin. In particular, the cathepsin inhibitors of the present invention can improve motor-impairment due to neurodegenerative diseases such as Parkinson's disease.

The other agents known to be useful in the treatment of Parkinson's disease which can be administered in combination with the cathepsin inhibitors of the present invention include: L-DOPA; dopaminergic agonists such as quinpirole, ropinirole, pramipexole, pergolide and bromocriptine; MAO-B inhibitors such as deprenyl and selegiline; DOPA decarboxylase inhibitors such as carbidopa and benserazide; and COMT inhibitors such as tolcapone and entacapone.

A preferred dosage for the administration of a compound of the present invention is about 0.001 to 500 mg/kg of body weight/day of a compound of the present invention or a pharmaceutically acceptable salt or ester thereof. An especially preferred dosage is about 0.01 to 25 mg/kg of body weight/day of a compound of the present invention or a pharmaceutically acceptable salt or ester thereof.

The phrases “effective amount” and “therapeutically effective amount” mean that amount of a compound of the present invention, and other pharmacological or therapeutic agents described herein, that will elicit a biological or medical response of a tissue, a system, or a subject (e.g., animal or human) that is being sought by the administrator (such as a researcher, doctor or veterinarian) which includes alleviation of the symptoms of the condition or disease being treated and the prevention, slowing or halting of progression of one or more of the presently claimed diseases. The formulations or compositions, combinations and treatments of the present invention can be administered by any suitable means which produce contact of these compounds with the site of action in the body of, for example, a mammal or human.

For administration of pharmaceutically acceptable salts of the above compounds, the weights indicated above refer to the weight of the acid equivalent or the base equivalent of the therapeutic compound derived from the salt.

As described above, this invention includes combinations comprising an amount of at least one compound of the presently claimed methods or a pharmaceutically acceptable salt or ester thereof, and an amount of one or more additional therapeutic agents listed above (administered together or sequentially) wherein the amounts of the compounds/treatments result in desired therapeutic effect.

When administering a combination therapy to a patient in need of such administration, the therapeutic agents in the combination, or a pharmaceutical composition or compositions comprising the therapeutic agents, may be administered in any order such as, for example, sequentially, concurrently, together, simultaneously and the like. The amounts of the various actives in such combination therapy may be different amounts (different dosage amounts) or same amounts (same dosage amounts). Thus, for illustration purposes, a compound of the present invention and an additional therapeutic agent may be present in fixed amounts (dosage amounts) in a single dosage unit (e.g., a capsule, a tablet and the like). A commercial example of such single dosage unit containing fixed amounts of two different active compounds is VYTORIN® (available from Merck Schering-Plough Pharmaceuticals, Kenilworth, N.J.).

If formulated as a fixed dose, such combination products employ the compounds of this invention within the dosage range described herein and the other pharmaceutically active agent or treatment within its dosage range. Compounds of the present invention may also be administered sequentially with known therapeutic agents when a combination formulation is inappropriate. The invention is not limited in the sequence of administration; compounds of the present invention may be administered either prior to or after administration of the known therapeutic agent. Such techniques are within the skills of persons skilled in the art as well as attending physicians.

The pharmacological properties of the compounds of this invention may be confirmed by a number of pharmacological assays (assays measuring cathepsin inhibition activity) as set forth in the experimental section.

While it is possible for the active ingredient to be administered alone, it is preferable to present it as a pharmaceutical composition. The compositions of the present invention comprise at least one active ingredient, as defined above, together with one or more acceptable carriers, adjuvants or vehicles thereof and optionally other therapeutic agents. Each carrier, adjuvant or vehicle must be acceptable in the sense of being compatible with the other ingredients of the composition and not injurious to the mammal in need of treatment.

Accordingly, this invention also relates to pharmaceutical compositions comprising at least one compound utilized in the presently claimed methods, or a pharmaceutically acceptable salt or ester thereof and at least one pharmaceutically acceptable carrier, adjuvant or vehicle.

For preparing pharmaceutical compositions from the compounds described by this invention, inert, pharmaceutically acceptable carriers can be either solid or liquid. Solid form preparations include powders, tablets, dispersible granules, capsules, cachets and suppositories. The powders and tablets may be comprised of from about 5 to about 95 percent active ingredient. Suitable solid carriers are known in the art, e.g., magnesium carbonate, magnesium stearate, talc, sugar or lactose. Tablets, powders, cachets and capsules can be used as solid dosage forms suitable for oral administration. Examples of pharmaceutically acceptable carriers and methods of manufacture for various compositions may be found in A. Gennaro (ed.), Remington's Pharmaceutical Sciences, 18^{th} Edition, (1990), Mack Publishing Co., Easton, Pa.

The term pharmaceutical composition is also intended to encompass both the bulk composition and individual dosage units comprised of more than one (e.g., two) pharmaceutically active agents such as, for example, a compound of the present invention and an additional agent selected from the lists of the additional agents described herein, along with any pharmaceutically inactive excipients. The bulk composition and each individual dosage unit can contain fixed amounts of the afore-said “more than one pharmaceutically active agents”. The bulk composition is material that has not yet been formed into individual dosage units. An illustrative dosage unit is an oral dosage unit such as tablets, pills and the like. Similarly, the herein-described method of treating a subject by administering a pharmaceutical composition of the present invention is also intended to encompass the administration of the afore-said bulk composition and individual dosage units.

Additionally, the compositions of the present invention may be formulated in sustained release form to provide the rate controlled release of any one or more of the components or active ingredients to optimize the therapeutic effects. Suitable dosage forms for sustained release include layered tablets containing layers of varying disintegration rates or controlled release polymeric matrices impregnated with the active components and shaped in tablet form or capsules containing such impregnated or encapsulated porous polymeric matrices.

Liquid form preparations include solutions, suspensions and emulsions. As an example may be mentioned water or water-propylene glycol solutions for parenteral injection or addition of sweeteners and opacifiers for oral solutions, suspensions and emulsions. Liquid form preparations may also include solutions for intranasal administration.

Aerosol preparations suitable for inhalation may include solutions and solids in powder form, which may be in combination with a pharmaceutically acceptable carrier, such as an inert compressed gas, e.g. nitrogen.

Also included are solid form preparations that are intended to be converted, shortly before use, to liquid form preparations for either oral or parenteral administration. Such liquid forms include solutions, suspensions and emulsions.

The compounds of the invention may also be deliverable transdermally. The transdermal compositions can take the form of creams, lotions, aerosols and/or emulsions and can be included in a transdermal patch of the matrix or reservoir type as are conventional in the art for this purpose.

The compounds of this invention may also be delivered subcutaneously.

Preferably the compound is administered orally.

Preferably, the pharmaceutical preparation is in a unit dosage form. In such form, the preparation is subdivided into suitably sized unit doses containing appropriate quantities of the active component, e.g., an effective amount to achieve the desired purpose.

The quantity of active compound in a unit dose of preparation may be varied or adjusted from about 1 mg to about 100 mg, preferably from about 1 mg to about 50 mg, more preferably from about 1 mg to about 25 mg, according to the particular application.

The actual dosage employed may be varied depending upon the requirements of the patient and the severity of the condition being treated. Determination of the proper dosage regimen for a particular situation is within the skill of the art. For convenience, the total daily dosage may be divided and administered in portions during the day as required.

The amount and frequency of administration of the compounds of the present invention and/or the pharmaceutically acceptable salts or esters thereof will be regulated according to the judgment of the attending clinician considering such factors as age, condition and size of the patient as well as severity of the symptoms being treated. A typical recommended daily dosage regimen for oral administration can range from about 1 mg/day to about 500 mg/day, preferably 1 mg/day to 200 mg/day, in two to four divided doses.

Cathepsin Assay Protocols:

Cathepsin G:

Materials:

1. 96-well Round or V-bottom microplates for dilutions
2. 96-well COSTAR UV microplates for assay (COSTAR cat.#3635)
3. Bio-Spin 6 Chromatography Columns (BIO-RAD cat.#732-6002).
4. 12-75 mm test tubes
5. Falcon 50 ml tubes
6. Falcon 14 ml tubes
7. 1.5 ml eppendorf tube
8. ≦10 ml buffer reservoir
   Reagents:
1. Enzyme: Cathepsin G, M.W.23,500, 10 μM stock.
2. Substrate:Suc-Ala-Ala-Pro-Phe-pNA, M.W. 624.65, prep. 100 mM [62.465 mg/ml] stock.
3. Buffer: [50 mM HEPES, 500 mM NaCl, 5 μM EDTA, pH 7.14], 5 μM DTT
   Instrument Settings: SpectraMax Plus
1. Kinetic assay reading@405 nm
2. Read interval: 30 seconds
3. Time: 1 hour
4. OD Min: −0.001
5. OD Max: 0.5
6. Data Mode: Absorbance
7. e405=194.078 μM/O.D.
   Procedure:
1. Add 73 μl of buffer to all wells.
2. Add 10 μl of buffer to (−E) controls.
3. Add 7 μl of sample and/or DMSO for controls to appropriate wells.
4. Prep. substrate: 100 mM (62.465 mg/ml) stock in 100% DMSO.
5. Dilute 1:20 in buffer. 75 μl in 1.425 ml. 5 mM final.
6. Add 20 μl of 5 mM substrate to all wells. 500 μM final.
7. Dilute enzyme (1:50) in buffer; Add 120 μl to 5,880 μl buffer. 2×[200 nM] final.
8. Add 100 μl of 2× [200 nM] enzyme to assigned wells, except (−E) controls. [100 nM] final.
11. Vortex 20 seconds. Read@ 405 nM every 30 seconds for 1 hour on SpectraMax Plus
Endpoint assay reading plate@ 600 nM. Used for the detection of precipitation.
   Cathepsin H:
   Materials:
1. 96-well Round or V-bottom microplates for dilutions
2. 96-well COSTAR UV microplates for assay (COSTAR cat.#3635)
3. Bio-Spin 6 Chromatography Columns (BIO-RAD cat.#732-6002)
4. 12-75 mm test tubes
5. Falcon 50 ml tubes
6. Falcon 14 ml tubes
7. 1.5 ml eppendorf tube
8. ≧10 ml buffer reservoir
   Reagents:
1. Enzyme: Cathepsin H, M.W.28,000, 9.07 μM stock.
2. Substrate:H-Arg-pNA, M.W. 367.2, prep. 50 mM [18.36 mg/ml] stock.
3. Buffer: [100 mM NaP04, 1 mM EDTA, 1 mM DTT, pH 6.5
   Instrument Settings: SpectraMax Plus
1. Kinetic assay reading@410 nM
2. Read interval: 30 seconds
3. Time: 1 hour
4. OD Min: −0.001
5. OD Max: 0.05
6. Data Mode: Absorbance
7. e410=188.415 μM/O.D. Km=108±5 μM
   Procedure:
1. Prep. substrate: 50 mM (18.36 mg/ml) stock in 100% DMSO.
2. Add 73 μl of buffer to all wells.
3. Add 7 μl of sample to appropriate wells: a.) 100% DMSO for (+E) & (−E) controls.
4. Add 100 μl of buffer to (−E) controls.
5. Dilute substrate (1:20) in buffer; Add 140 μl to 2.66 ml buffer while vortexing. [2.5 mM] final.
6. Add 20 μl of substrate to all wells. Vortex 3-5 seconds. [250 μM] final.
7. Dilute enzyme (1:64.8) in buffer; Add 179 μl to 11.418 ml buffer. 2× [140 nM] final.
8. Add 100 μl of 2× [140 nM] enzyme to assigned wells, except (−E) controls. [70 nM] final.
9. Read@410 nM every 30 seconds for 1 hour on SpectraMax Plus
   Cathepsin L:
   Materials:
1. 96-well Round or V-bottom microplates for dilutions
2. 96-well COSTAR UV microplates for assay (COSTAR cat.#3635)
3. Falcon 50 ml tubes
4. Falcon 14 ml tubes
5. 1.5 ml eppendorf tube
6. 10 ml buffer reservoir
   Reagents:
1. Enzyme: Calbiochem #219402: Cathepsin L, M.W.29,000, 11.62 μM stock. Lot#B45683
2. Substrate: Bachem #L-1242: Z-Phe-Arg-pNA, M.W. 612.09, prep. 4.8 mM [2.938 mg/ml] stock in 100% DMSO.
3. Buffer: [100 mM NaP04, 1 mM EDTA, pH 5.5] Supplement buffer w/1 mM DTT, day of assay.
   Experiment #1:
Instrument settings: SpectraMax Plus microtiter plate reader (Molecular Devices, Sunnydale, Calif.)
1. Kinetic assay reading@410 nM
2. Read interval: 30 seconds
3. Time: 1 hour
4. OD Min: −0.001
5. OD Max: 0.05
6. Data Mode: Absorbance
7. ε₄₁₀=161.311 μM/O.D. Km=56±8 μM
   Procedure:
1. Add 88 μl of buffer to all wells.
2. Add 7 μl of sample or 100% DMSO to appropriate wells.
3. Add 100 μl of buffer to (−E) controls.
5. Add 5 μ 4.8 mM] substrate to all wells. [120 μM] final.
6. Vortex 3-5 seconds.
7. Dilute enzyme (1:483.87) in buffer; Add 24.8 μl to 12,000 μl buffer. 2×[24 nM] final.
8. Add 100 μl of 2× [24 nM] enzyme to assigned wells, except (−E) controls. [12 nM] final.
9. Read@410 nM every 30 seconds for 1 hour on SpectraMax Plus
   Experiment #2:
Endpoint assay reading plate@600 nM. Used for the detection of precipitation.
Calculations of Ki are performed in accordance with the following reference:
Analytical Biochemistry (1999) 270, 268-270.

The tables below sets forth cathepsin L inhibitory activities for representative compounds.

| | Human Liver | | | |
|---|---|---|---|---|
| | Cathepsin L | | | |
| | Ki* | ± Ki* | N | 95% |
| MOLSTRUCTURE | [nM] | [nM] | = | (fold) |
| 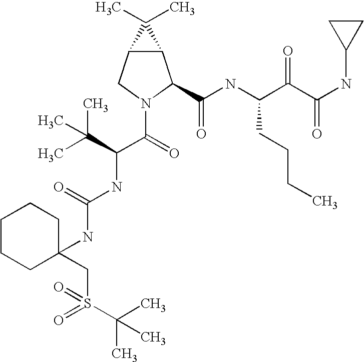 | 7 | 2 | 2 | 2.5 |
| 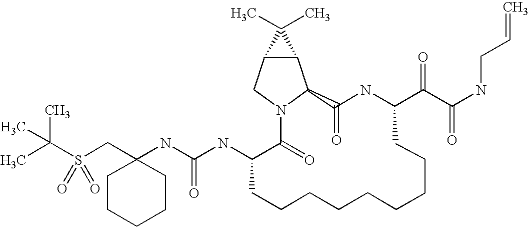 | 10 | 2 | 5 | 2.0 |
| 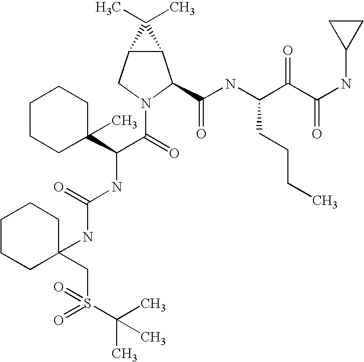 | 11 | 1 | 2 | 2.5 |
| 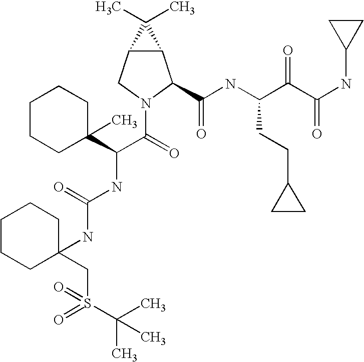 | 12 | 3 | 2 | 2.5 |
| 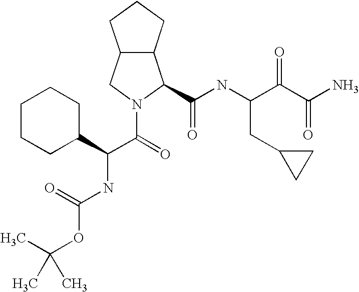 | 14 | 4 | 2 | 2.5 |
| 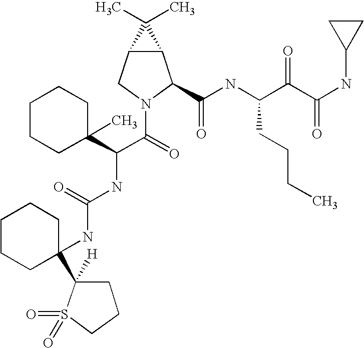 | 20 | 3 | 2 | 2.5 |
| 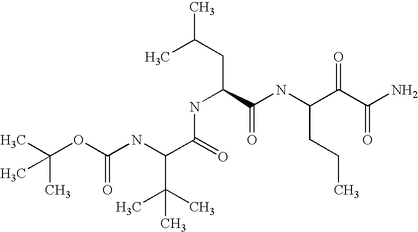 | 20 | 10 | 5 | 2.5 |
| 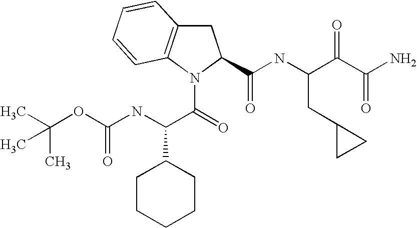 | 20 | 10 | 7 | 2.5 |
| 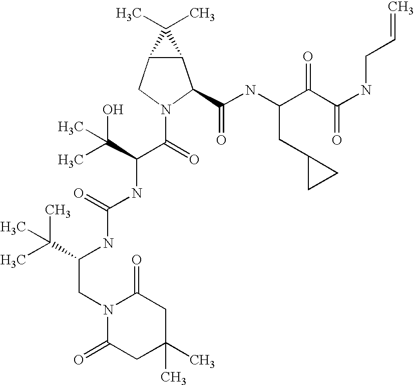 | 20 | 4 | 7 | 2.0 |
| 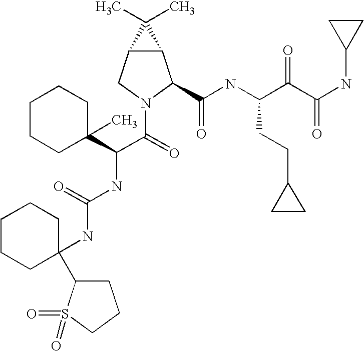 | 24 | 2 | 2 | 2.5 |
| 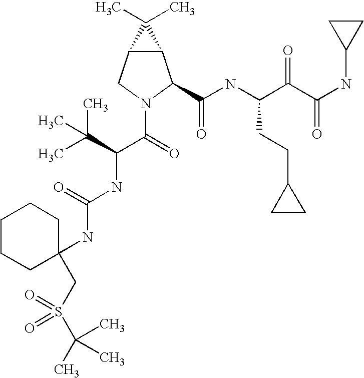 | 25 | 2 | 2 | 2.5 |
| 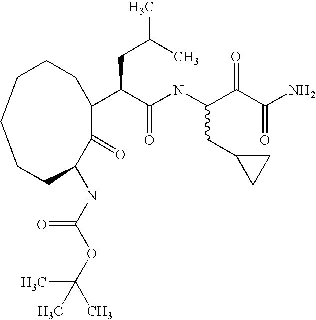 | 25 | 10 | 6 | 2.5 |
| 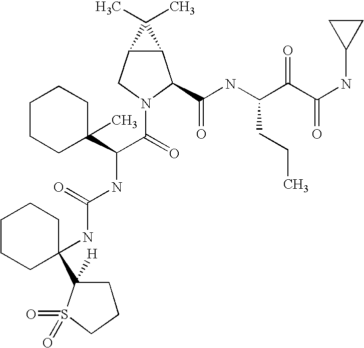 | 31 | 4 | 2 | 2.5 |
| 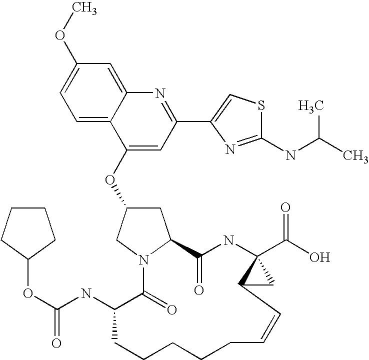 | 38 | 2 | 5 | 1.5 |
| 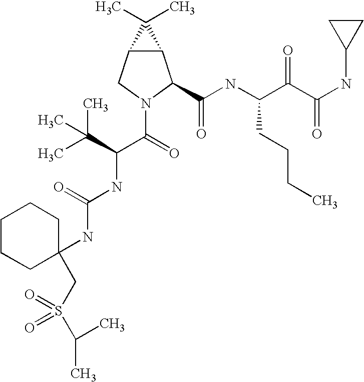 | 38 | 5 | 6 | 2.0 |
| 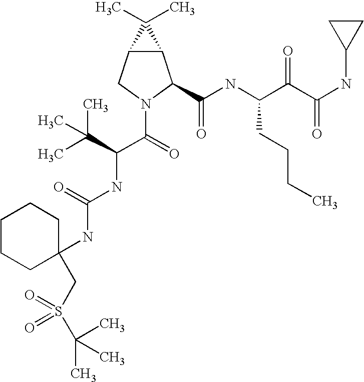 | 40 | 15 | 8 | 1.5 |
| 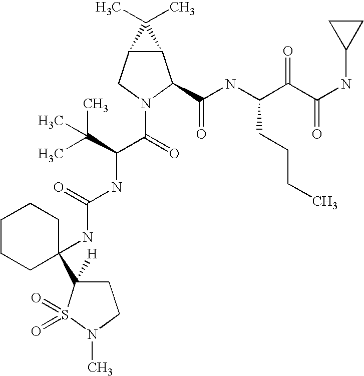 | 40 | 9 | 2 | 2.5 |
| 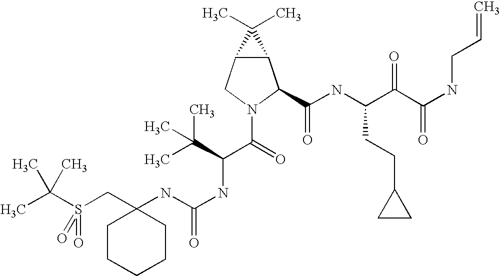 | 40 | 20 | | 2.0 |
| 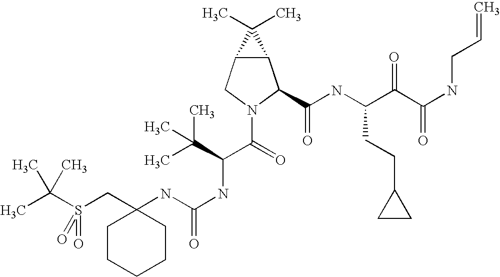 | 45 | 14 | 8 | 1.5 |
| 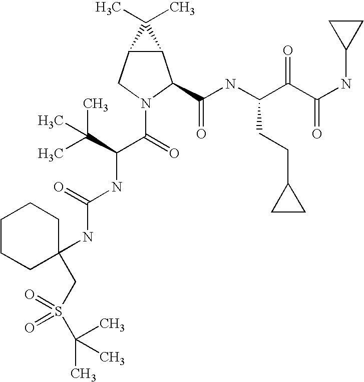 | 47 | 10 | 8 | 1.5 |
| 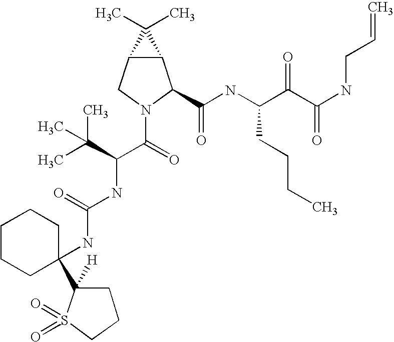 | 49 | 10 | 8 | 1.5 |
| 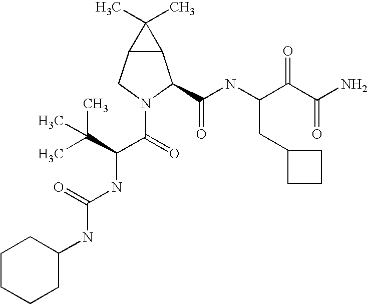 | 63 | 14 | 4 | 1.5 |
| 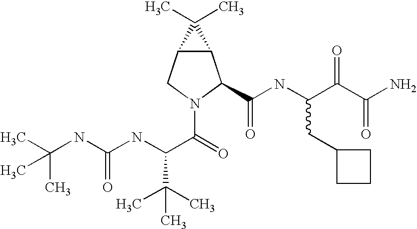 | 76 | 12 | 8 | 1.5 |
| 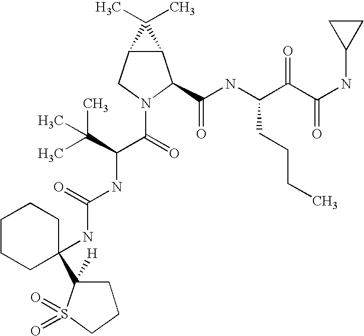 | 80 | 10 | | 2.0 |
| 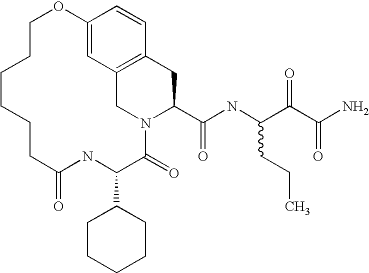 | 80 | 20 | 3 | 2.5 |
| 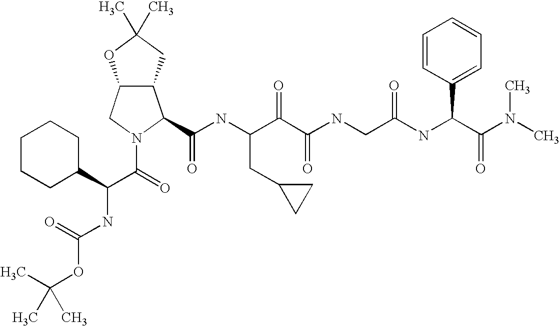 | 97 | 23 | 6 | 1.5 |
| 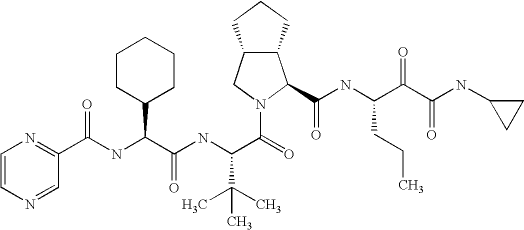 | 104 | 18 | 8 | 1.5 |
| 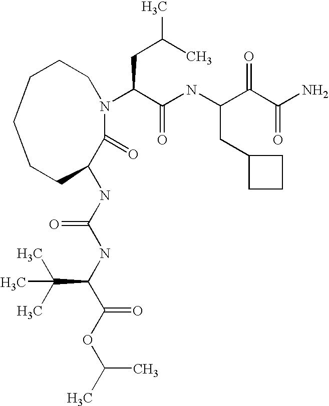 | 120 | 50 | 4 | 2.5 |
| 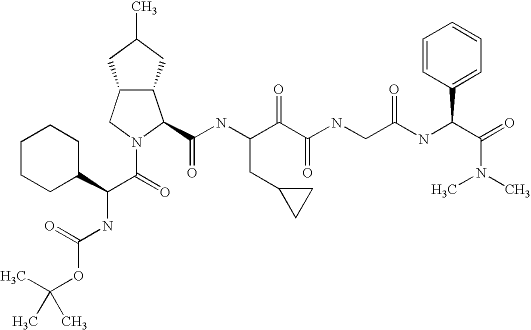 | 120 | 4 | 2 | 2.5 |
| 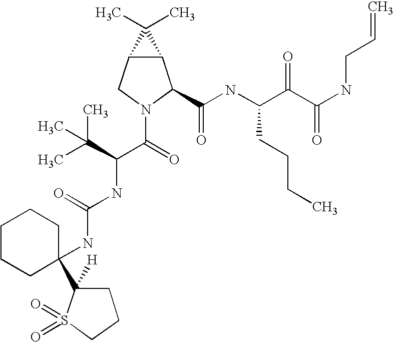 | 140 | 40 | | 2.0 |
| 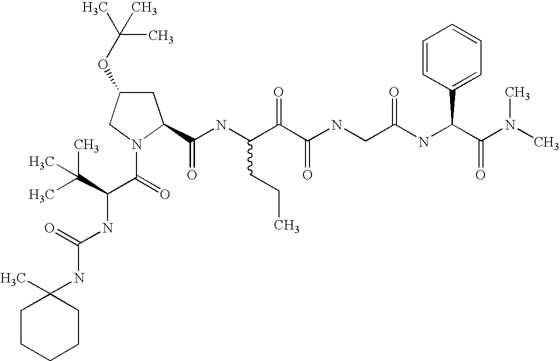 | 170 | 25 | 3 | 2.5 |
| 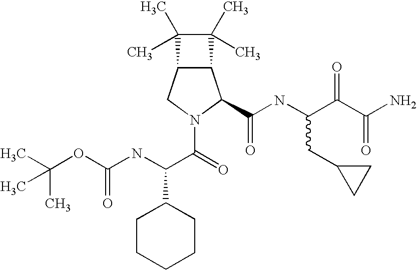 | 220 | 60 | 5 | 2.5 |
| 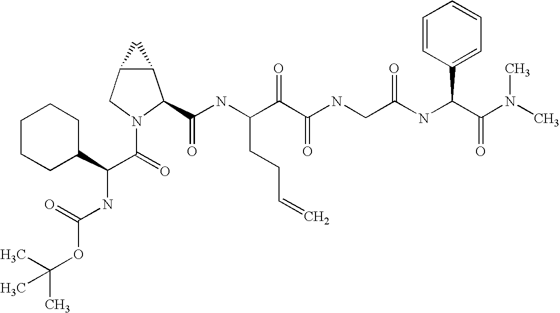 | 270 | 20 | 3 | 2.5 |
| 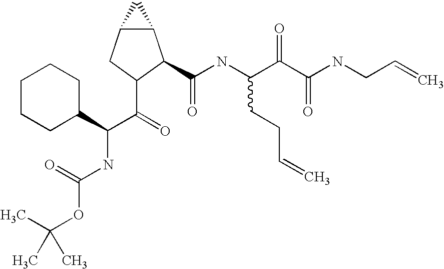 | 1,500 | 700 | 2 | 2.5 |
| 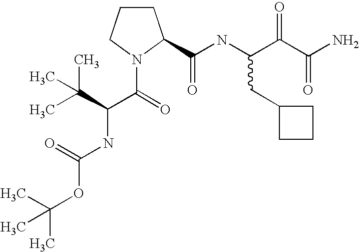 | 1,900 | 400 | 2 | 2.5 |
| 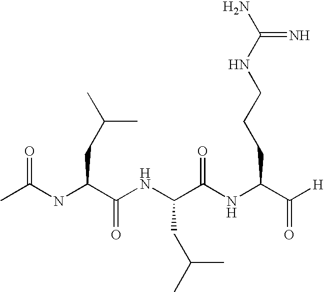 | 9 | 3 | 4 | 1.5 |
| 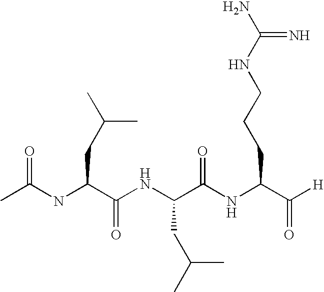 | 1.9 | 0.3 | 8 | 1.5 |
| 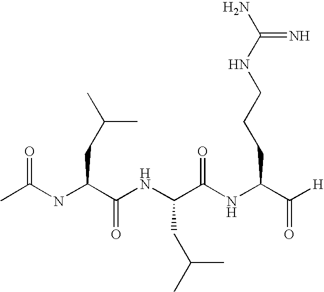 | 8 | 3 | 8 | 2.0 |
| 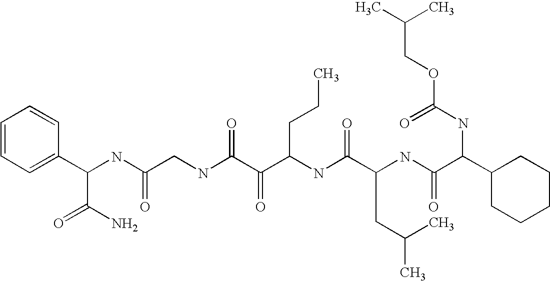 | 2 | 1 | 4 | 2.5 |
| 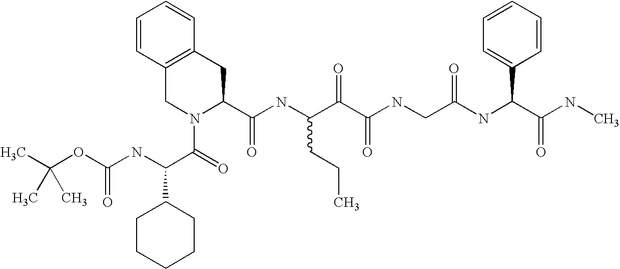 | 36 | 15 | 7 | 2.5 |
| 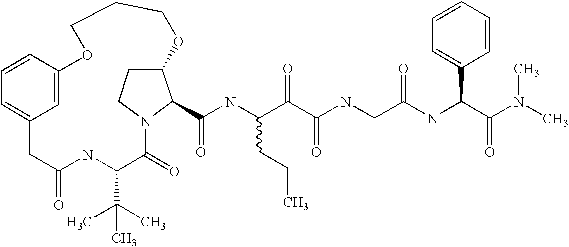 | 70 | 30 | 6 | 2.5 |
| 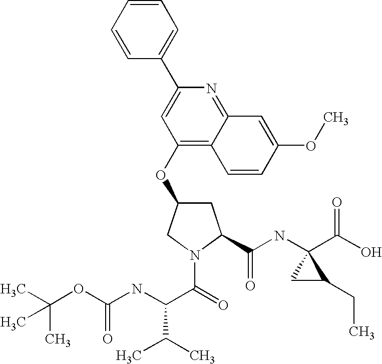 | 100 | 30 | 4 | 2.5 |
| 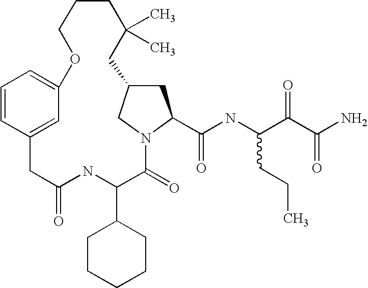 | 17 | 7 | 6 | 2.5 |
| 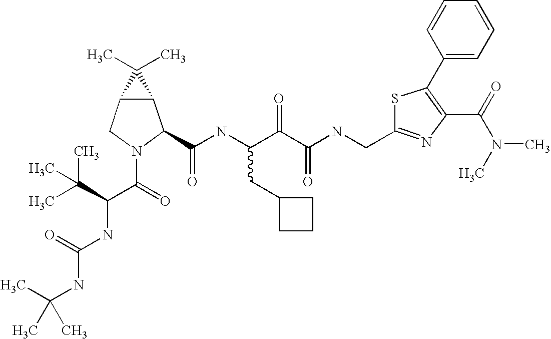 | 190 | 30 | 4 | 2.5 |
| 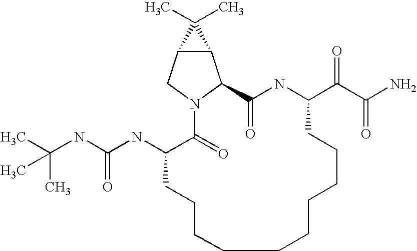 | 300 | 100 | 4 | 2.5 |
| 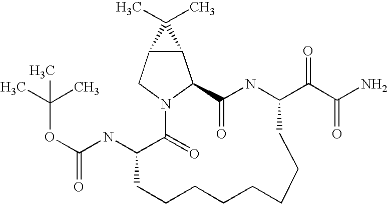 | 550 | 220 | 3 | 2.5 |
| 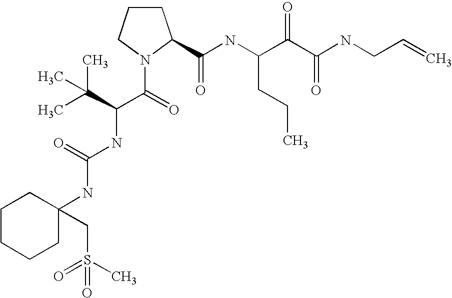 | 740 | 60 | 2 | 2.5 |
| 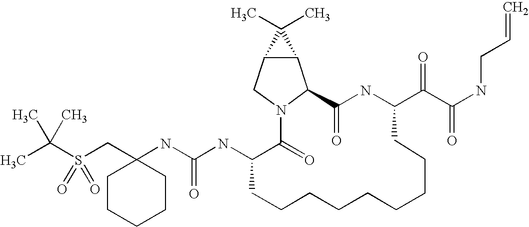 | 60 | 20 | 5 | 2.5 |
| 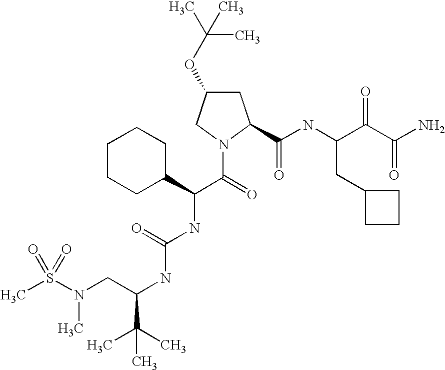 | 110 | 30 | 2 | 2.5 |
| 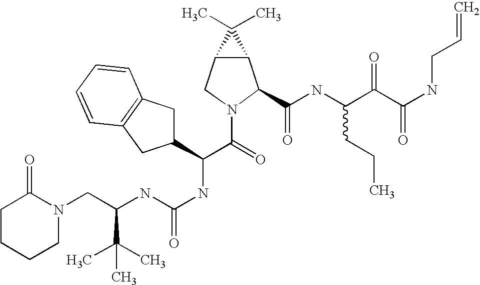 | 140 | 60 | 4 | 2.5 |
| 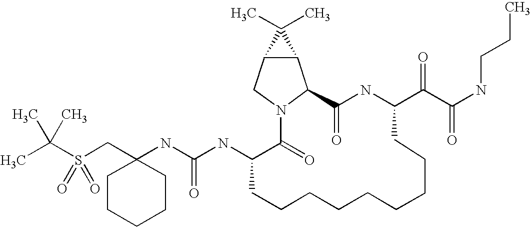 | 330 | 150 | 3 | 2.5 |
| 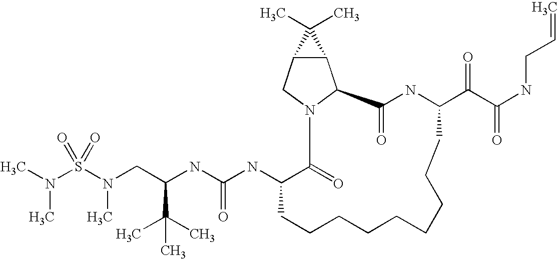 | 370 | 180 | 4 | 2.5 |
| 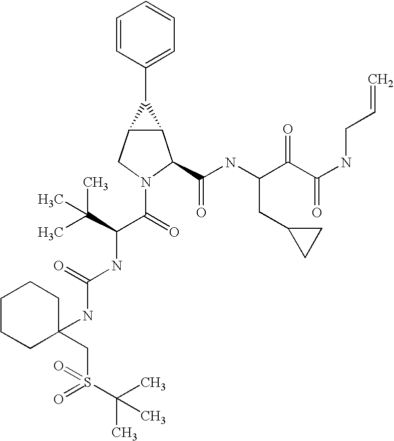 | 350 | 30 | 2 | 2.5 |
| 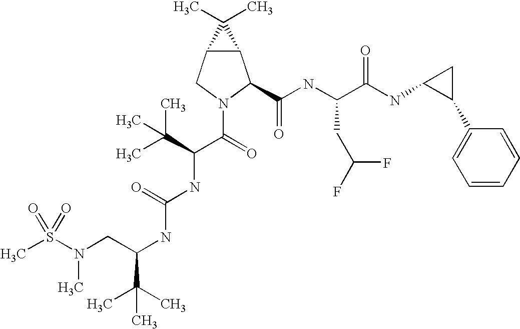 | 530 | 230 | 2 | 2.5 |
| 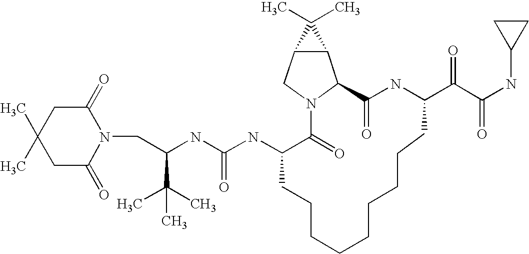 | 250 | 120 | 4 | 2.5 |
| 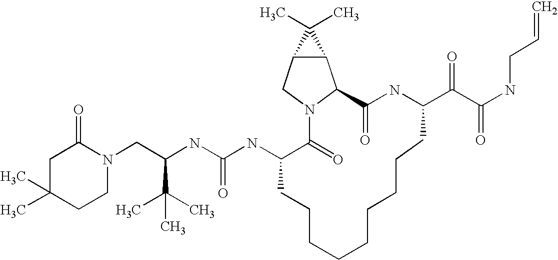 | 366 | 2 | 2 | 2.5 |
| 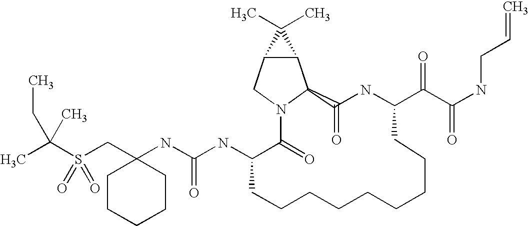 | 230 | 80 | 4 | 2.5 |
| 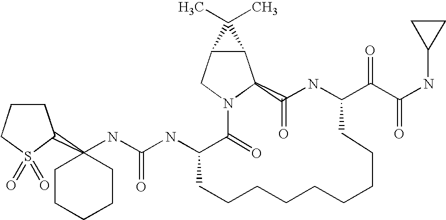 | 110 | 20 | 4 | 2.5 |
| 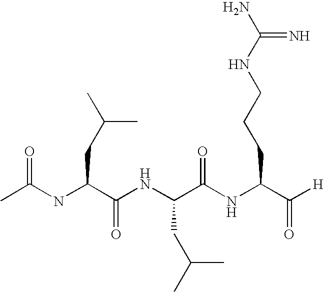 | 4.8 | 0.3 | 8 | 2.0 |

95% = 95% Confidence boundaries
N = Number of observations
** = Precipitation observed

Variations in Ki values for the same compound are due in part to different batches of the compound being tested.

It will be appreciated by those skilled in the art that changes could be made to the embodiments described above without departing from the broad inventive concept thereof. It is understood, therefore, that this invention is not limited to the particular embodiments disclosed, but it is intended to cover modifications that are within the spirit and scope of the invention, as defined by the appended claims.

Each document (including granted patents, published patent applications, and nonpatent publications such as journal articles) referred to in this application is incorporated in its entirety by reference for all purposes.

What is claimed is:

1. A method of inhibiting cathepsin activity in a subject in need thereof comprising administering to said subject an effective amount of at least one compound of formula I 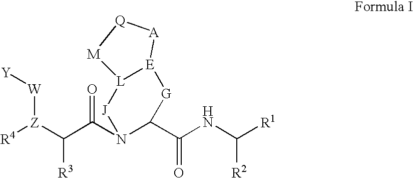
or a pharmaceutically acceptable salt, solvate or ester thereof;
wherein:
Y is selected from the group consisting of the following moieties: alkyl, alkyl-aryl, heteroalkyl, heteroaryl, aryl-heteroaryl, alkyl-heteroaryl, cycloalkyl, alkyloxy, alkyl-aryloxy, aryloxy, heteroaryloxy, heterocycloalkyloxy, cycloalkyloxy, alkylamino, arylamino, alkyl-arylamino, arylamino, heteroarylamino, cycloalkylamino and heterocycloalkylamino, with the proviso that Y maybe optionally substituted with X¹¹ or X¹²;
X¹¹ is alkyl, alkenyl, alkynyl, cycloalkyl, cycloalkyl-alkyl, heterocyclyl, heterocyclylalkyl, aryl, alkylaryl, arylalkyl, heteroaryl, alkylheteroaryl, or heteroarylalkyl, with the proviso that X¹¹ may be additionally optionally substituted with X¹²;
X¹² is hydroxy, alkoxy, aryloxy, thio, alkylthio, arylthio, amino, alkylamino, arylamino, alkylsulfonyl, arylsulfonyl, alkylsulfonamido, arylsulfonamido, carboxy, carbalkoxy, carboxamido, alkoxycarbonylamino, alkoxycarbonyloxy, alkylureido, arylureido, halogen, cyano, or nitro, with the proviso that said alkyl, alkoxy, and aryl may be additionally optionally substituted with moieties independently selected from X¹²;
R¹ is COR⁵ or B(OR)₂, wherein R⁵ is H, OH, OR⁸, NR⁹R¹⁰, CF₃, C₂F₅, C₃F₇, CF₂R⁶, R⁶, or COR⁷ wherein R⁷ is H, OH, OR⁸, CHR⁹R¹⁰, or NR⁹R¹⁰, wherein R⁶, R⁸, R⁹ and R¹⁰ are independently selected from the group consisting of H, alkyl, aryl, heteroalkyl, heteroaryl, cycloalkyl, cycloalkyl, arylalkyl, heteroarylalkyl, [CH(R^{1′})]ₚCOOR¹¹,[CH(R^{1′})]ₚCONR¹²R¹³,[CH(R^{1′})]ₚSO₂R¹¹,[CH(R^{1′})]ₚCOR¹¹,[CH(R^{1′})]ₚCH(OH)R¹¹,CH(R^{1′})CONHCH(R²)COOR¹¹,CH(R^{1′})CONHCH(R^{2′})CONR¹²R¹³,CH(R^{1′})CONHCH(R²)R′,CH(R^{1′})CONHCH(R^{2′})CONHCH(R^{3′})COOR¹¹,CH(R^{1′})CONHCH(R^{2′}) CONHCH(R^{3′})CONR¹²R¹³,CH(R^{1′})CONHCH(R^{2′})CONHCH(R^{3′})CONHCH(R^{4′})COOR¹¹,CH(R^{1′})CONHCH(R^{2′})CONHCH(R^{3′})CONHCH(R^{4′})CONR¹²R¹³,CH(R^{1′})CONHCH(R²)CONHCH(R^{3′})CONHCH(R^{4′})CONHCH(R^{5′})COOR¹¹ andCH(R^{1′})CONHCH(R^{2′})CONH CH(R^{3′})CONHCH(R^{4′})CONHCH(R^{5′}) CONR¹²R¹³, wherein R^{1′}, R^{2′}, R^{3′}, R^{4′}, R^{5′}, R¹¹, R¹², R¹³, and R′ are independently selected from the group consisting of H, alkyl, aryl, heteroalkyl, heteroaryl, cycloalkyl, alkyl-aryl, alkyl-heteroaryl, aryl-alkyl and heteroaralkyl;
Z is selected from O, N, CH or CR;
W maybe present or absent, and if W is present, W is selected from C═O, C═S, C(═N—CN), or SO₂;
Q maybe present or absent, and when Q is present, Q is CH, N, P, (CH₂)ₚ, (CHR)ₚ, (CRR′)ₚ, O, NR, S, or SO₂; and when Q is absent, M may be present or absent;
when Q and M are absent, A is directly linked to L;
A is O, CH₂, (CHR)ₚ, (CHR—CHR′)ₚ, (CRR′)ₚ, NR, S, SO₂ or a bond;
E is CH, N, CR, or a double bond towards A, L or G;
G may be present or absent, and when G is present, G is (CH₂)ₚ, (CHR)ₚ, or (CRR′)ₚ; and when G is absent, J is present and E is directly connected to the carbon atom in Formula I as G is linked to;
J maybe present or absent, and when J is present, J is (CH₂)ₚ, (CHR)ₚ, or (CRR′)ₚ, SO₂, NH, NR or O; and when J is absent, G is present and E is directly linked to N shown in Formula I as linked to J;
L may be present or absent, and when L is present, L is CH, CR, O, S or NR; and
when L is absent, then M may be present or absent; and if M is present with L being absent, then M is directly and independently linked to E, and J is directly and independently linked to E;
M may be present or absent, and when M is present, M is O, NR, S, SO₂, (CH₂)ₚ, (CHR)ₚ (CHR—CHR′)ₚ, or (CRR′)ₚ;
p is a number from 0 to 6; and
R, R′, R², R³ and R⁴ are independently selected from the group consisting of H; C₁-C₁₀ alkyl; C₂-C₁₀ alkenyl; C₃-C₈ cycloalkyl; C₃-C₈ heterocycloalkyl, alkoxy, aryloxy, alkylthio, arylthio, amino, amido, ester, carboxylic acid, carbamate, urea, ketone, aldehyde, cyano, nitro, halogen;
(cycloalkyl)alkyl and (heterocycloalkyl)alkyl, wherein said cycloalkyl is made of three to eight carbon atoms, and zero to six oxygen, nitrogen, sulfur, or phosphorus atoms, and said alkyl is of one to six carbon atoms; aryl; heteroaryl; alkyl-aryl; and alkyl-heteroaryl;
wherein said alkyl, heteroalkyl, alkenyl, heteroalkenyl, aryl, heteroaryl, cycloalkyl and heterocycloalkyl moieties may be optionally and chemically-suitably substituted, with said term “substituted” referring to optional and chemically-suitable substitution with one or more moieties selected from the group consisting of alkyl, alkenyl, alkynyl, aryl, aralkyl, cycloalkyl, heterocyclic, halogen, hydroxy, thio, alkoxy, aryloxy, alkylthio, arylthio, amino, amido, ester, carboxylic acid, carbamate, urea, ketone, aldehyde, cyano, nitro, sulfonamido, sulfoxide, sulfone, sulfonyl urea, hydrazide, and hydroxamate;
further wherein said unit N-C-G-E-L-J-N represents a five-membered or six-membered cyclic ring structure with the proviso that when said unit N-C-G-E-L-J-N represents a five-membered cyclic ring structure, or when the bicyclic ring structure in Formula I comprising N, C, G, E, L, J, N, A, Q, and M represents a five-membered cyclic ring structure, then said five-membered cyclic ring structure lacks a carbonyl group as part of the cyclic ring.

2. The method of claim 1, wherein said compound is selected from the group consisting of: 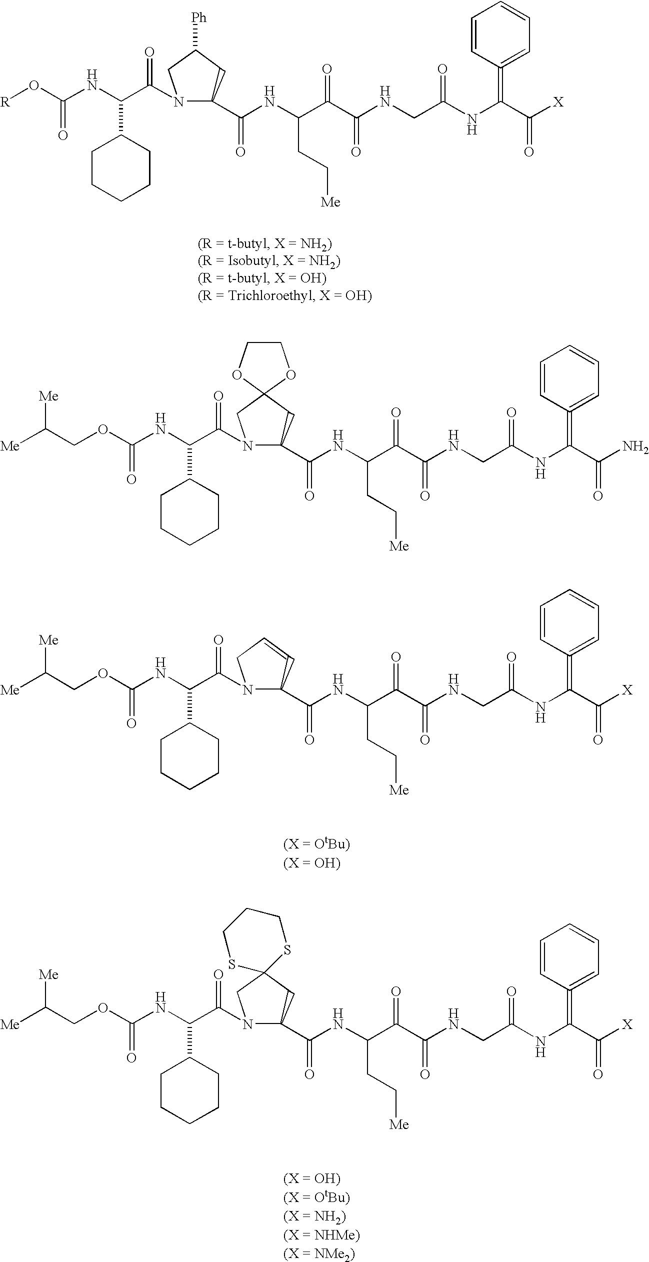 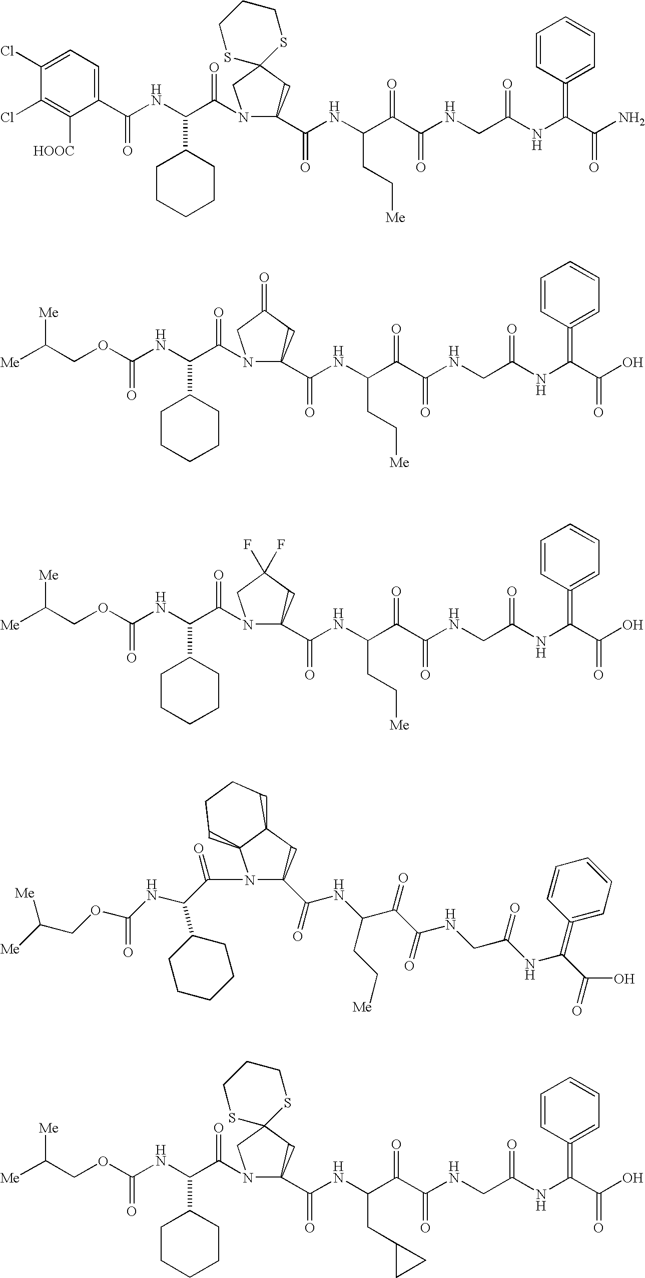 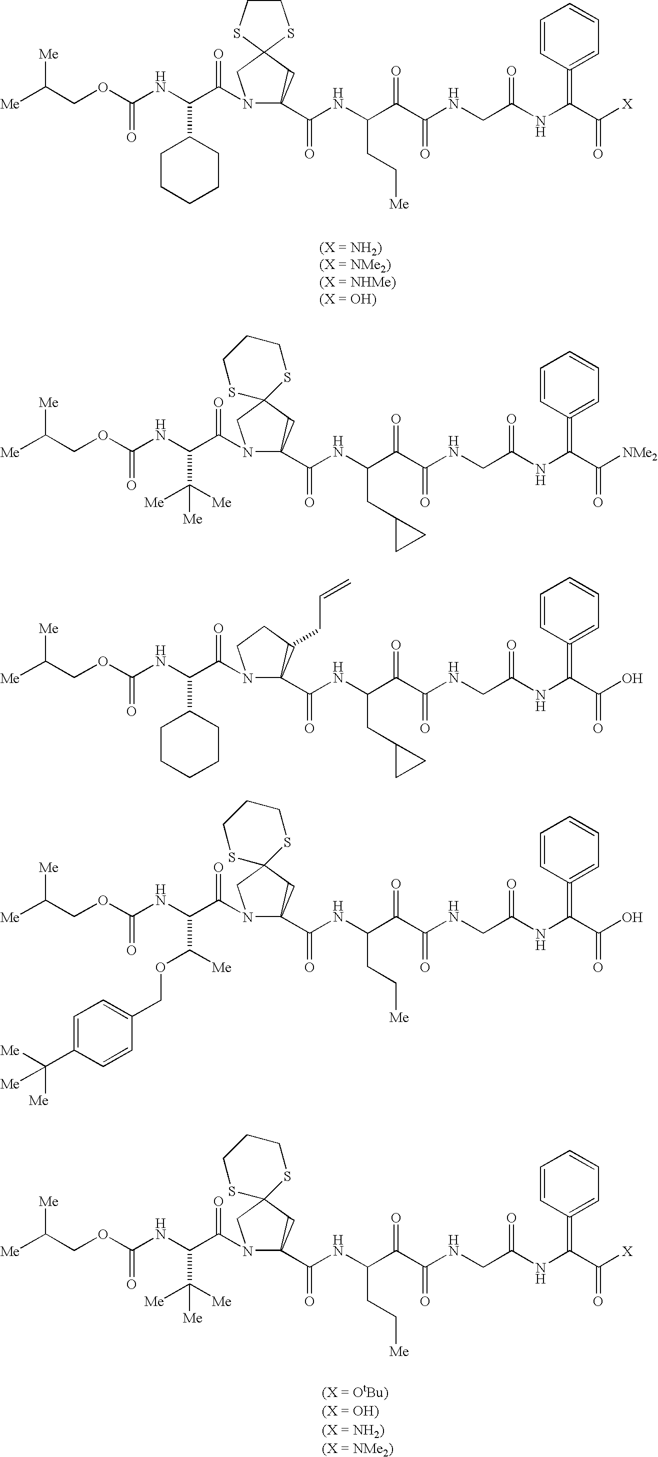 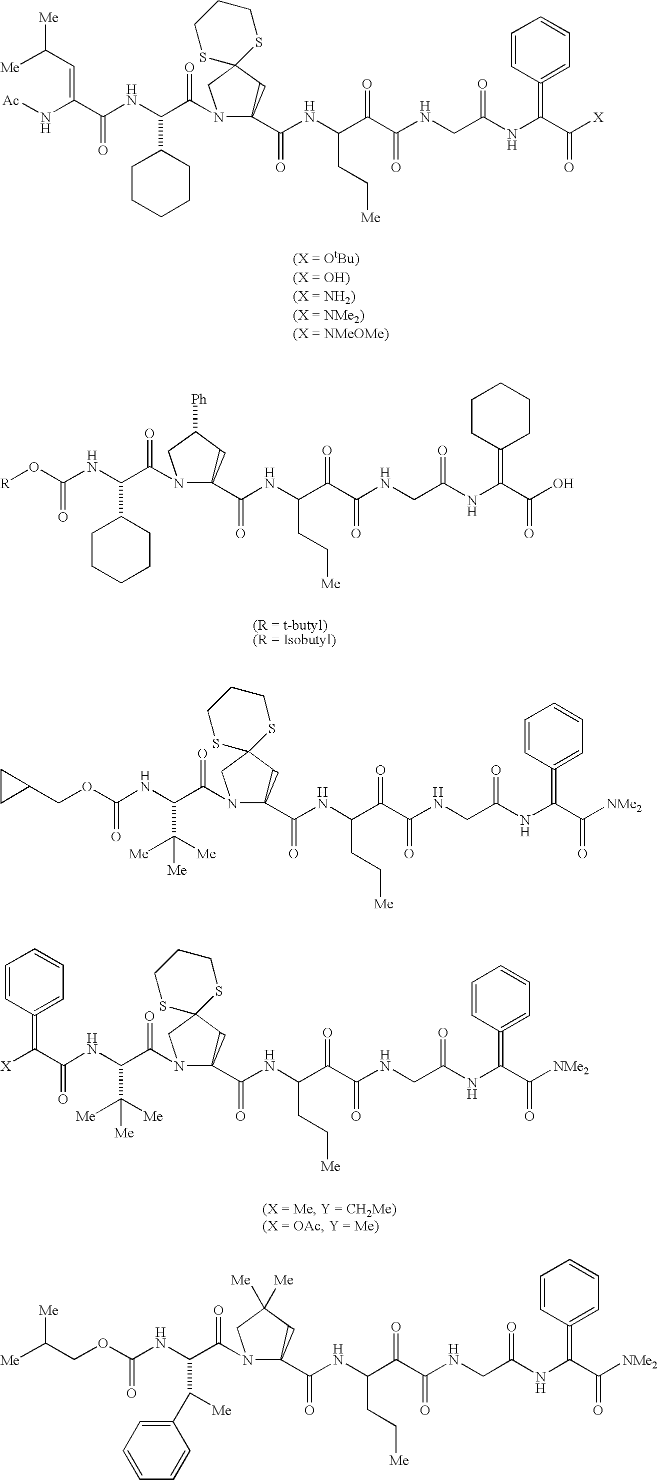 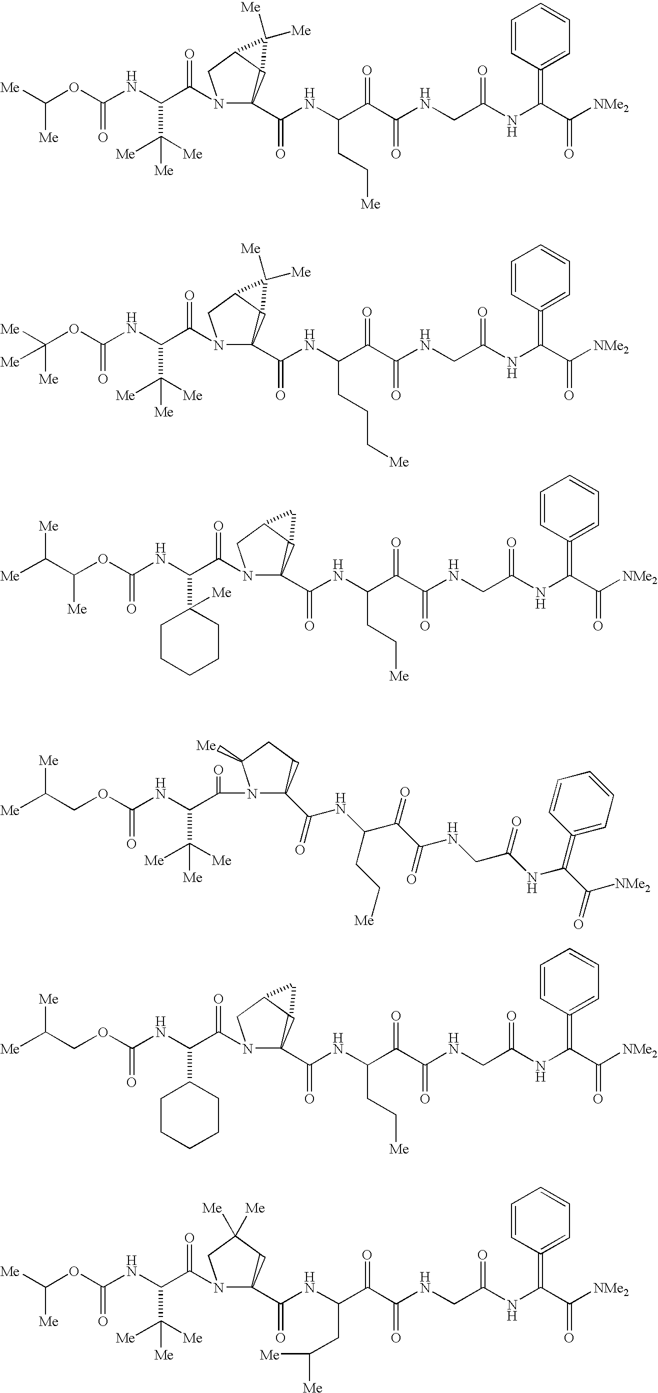 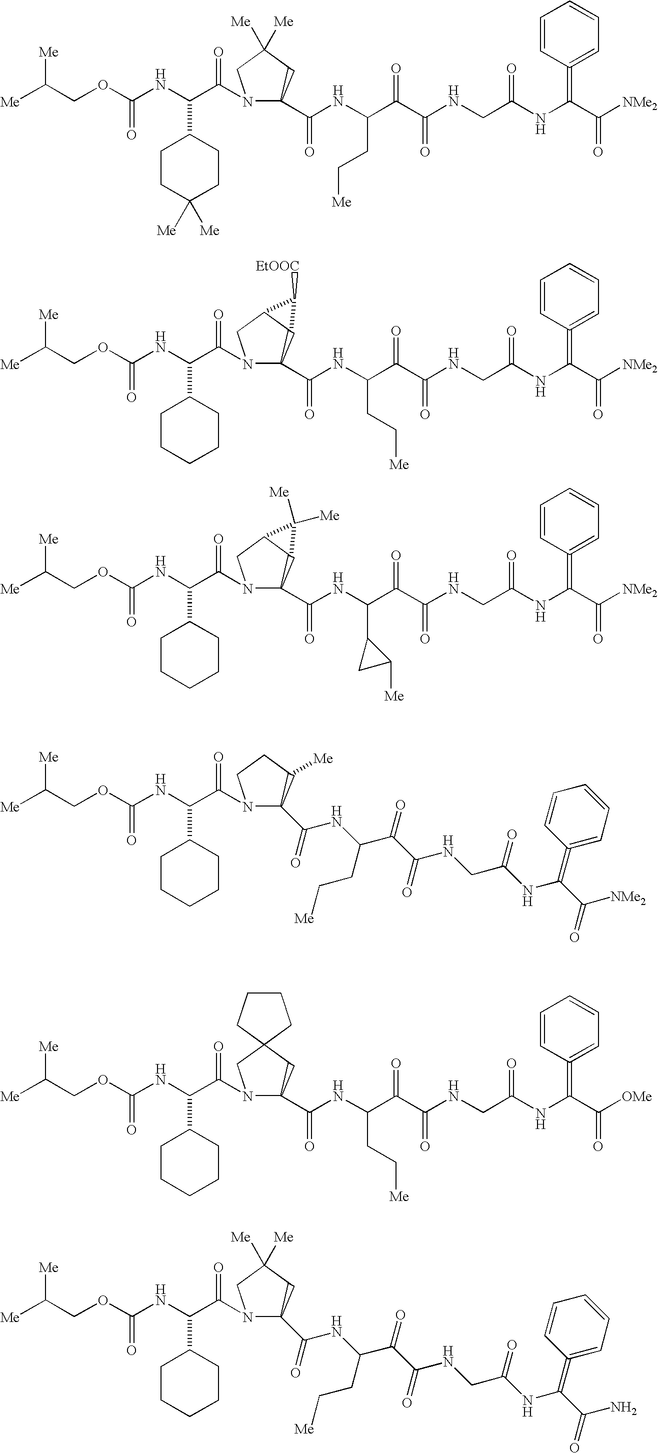 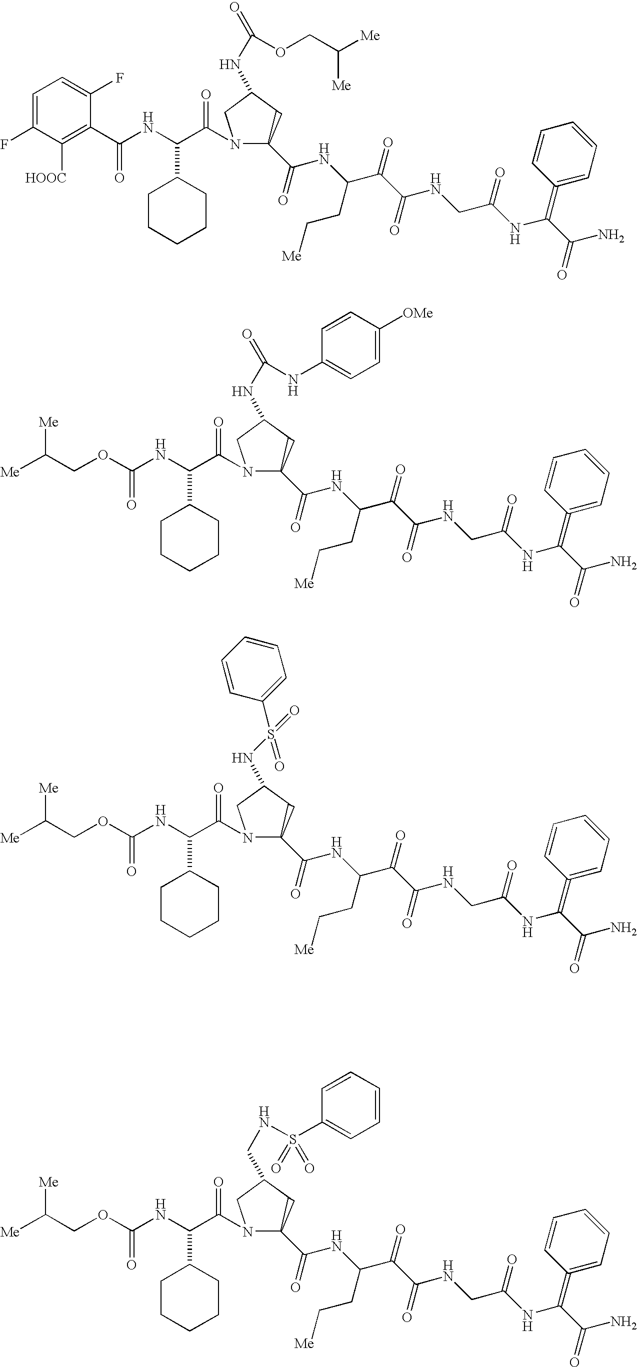 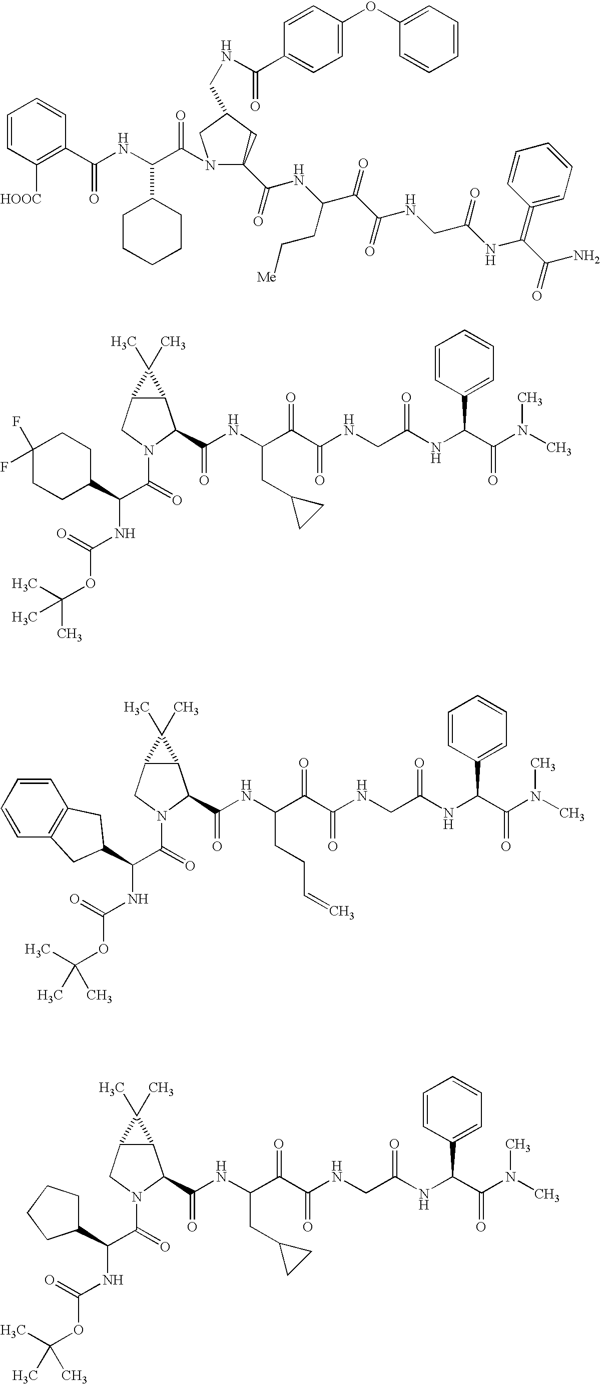 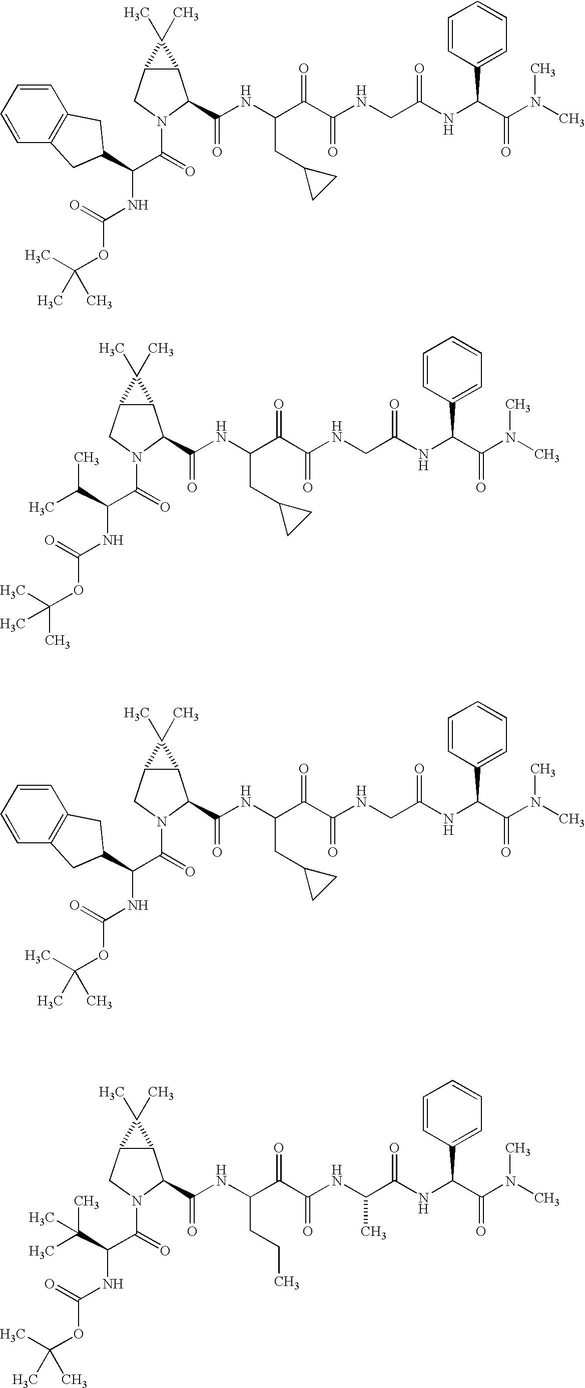 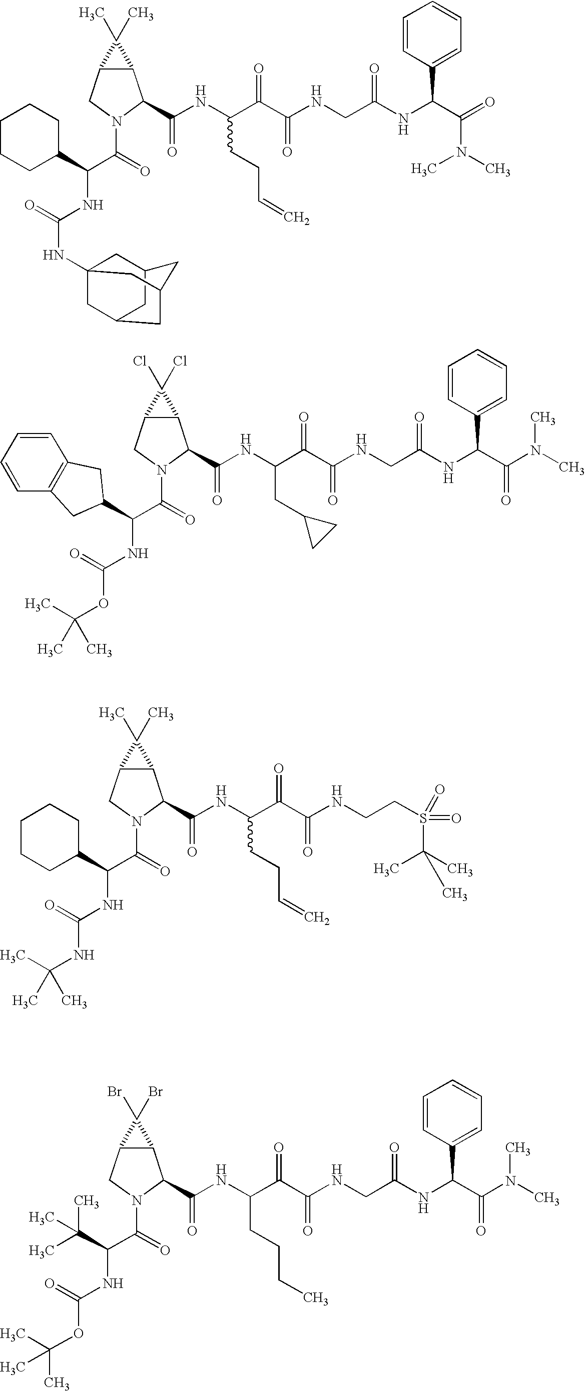 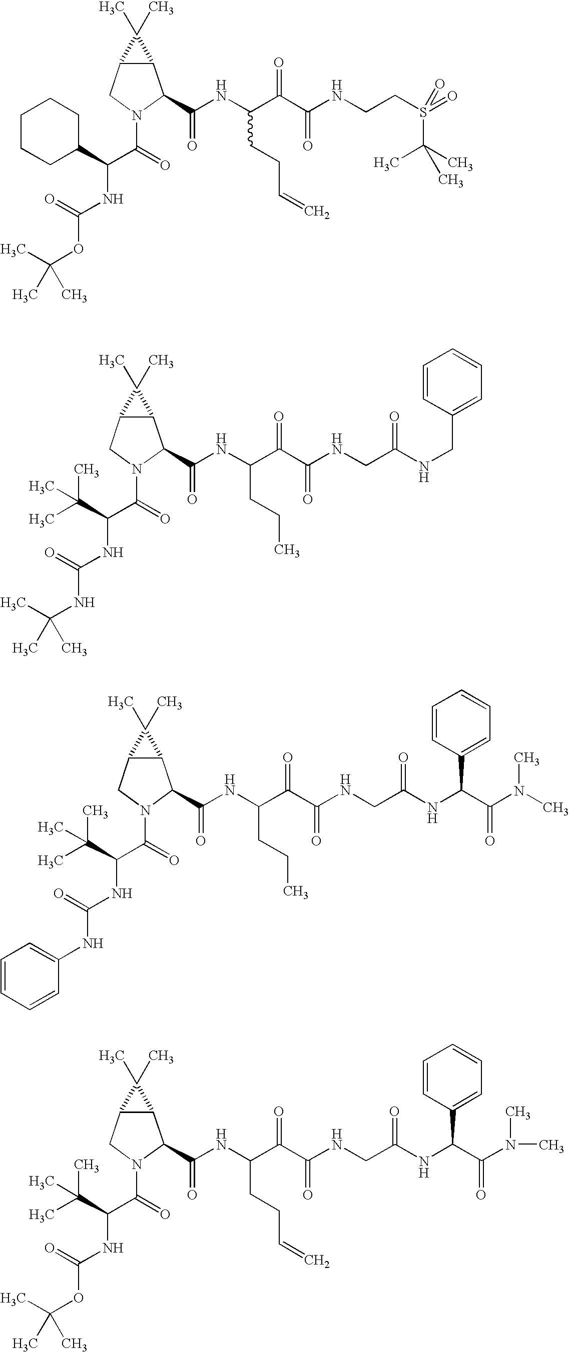 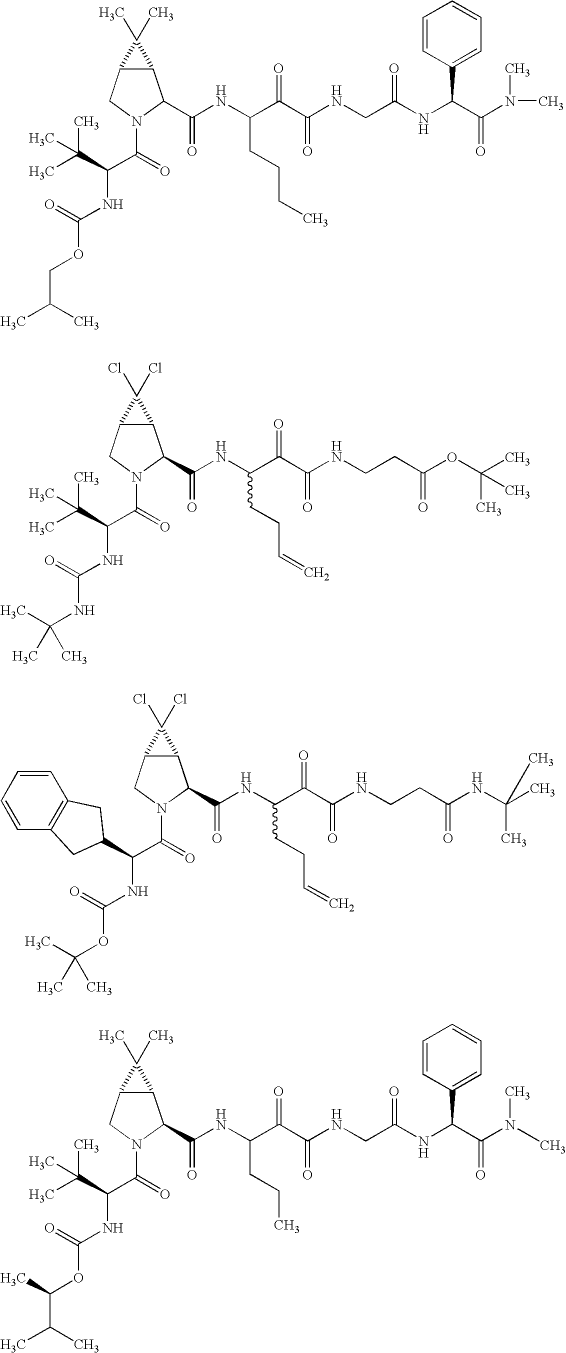 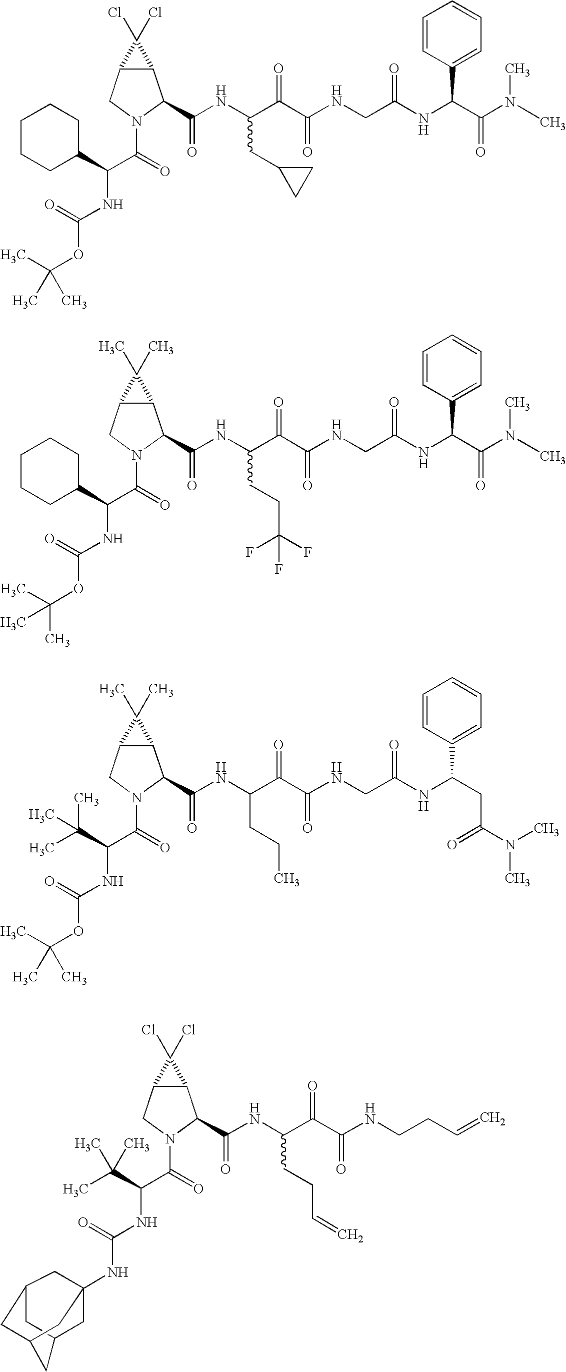 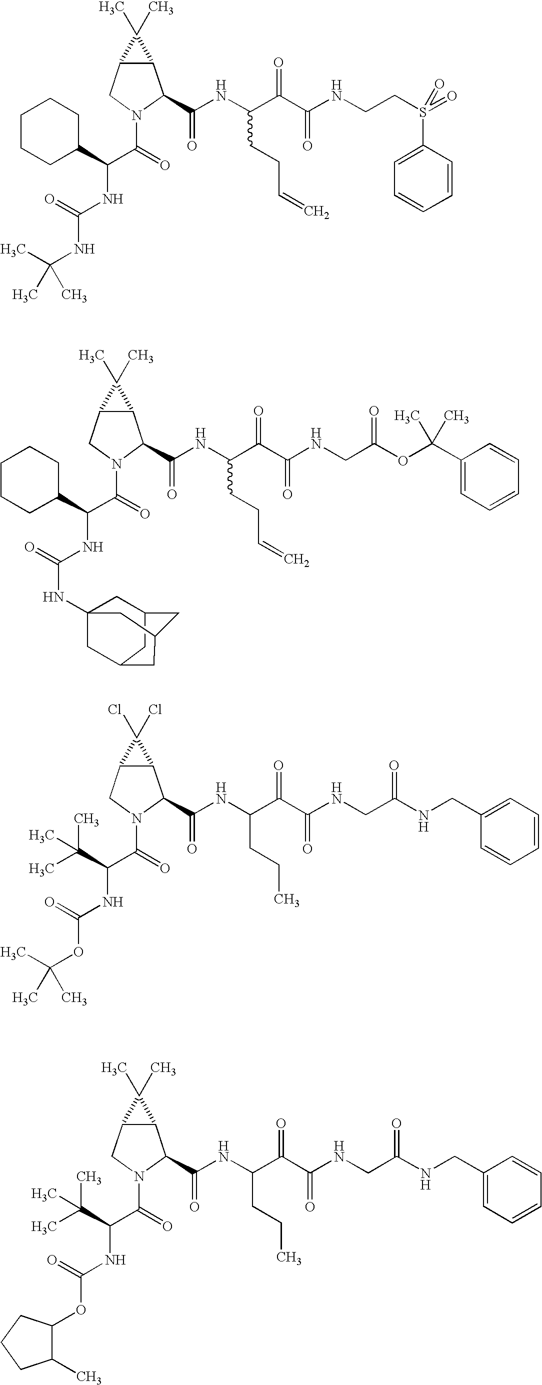 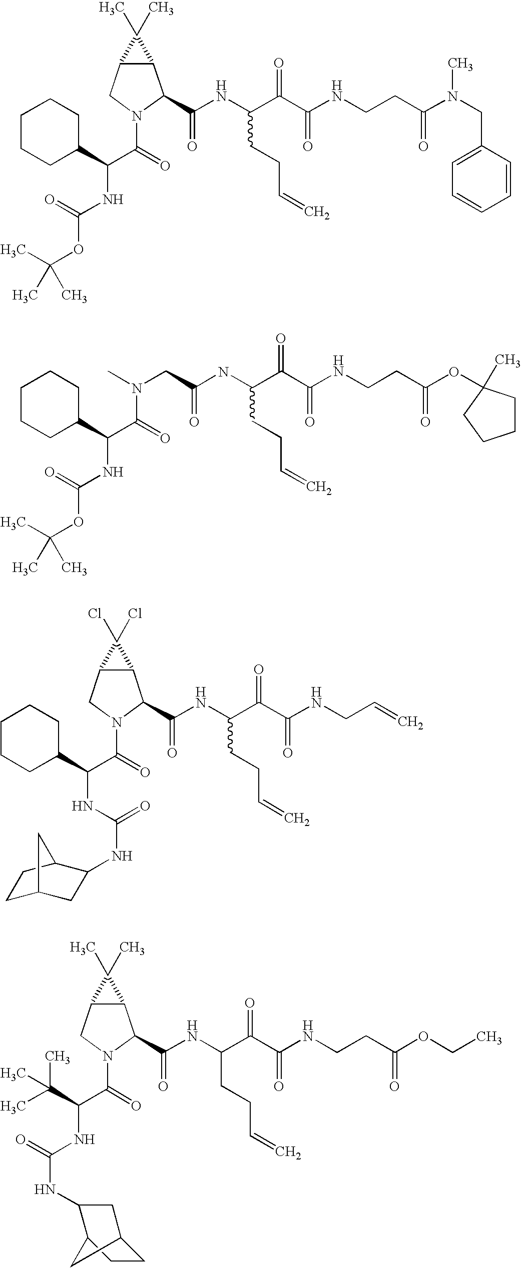 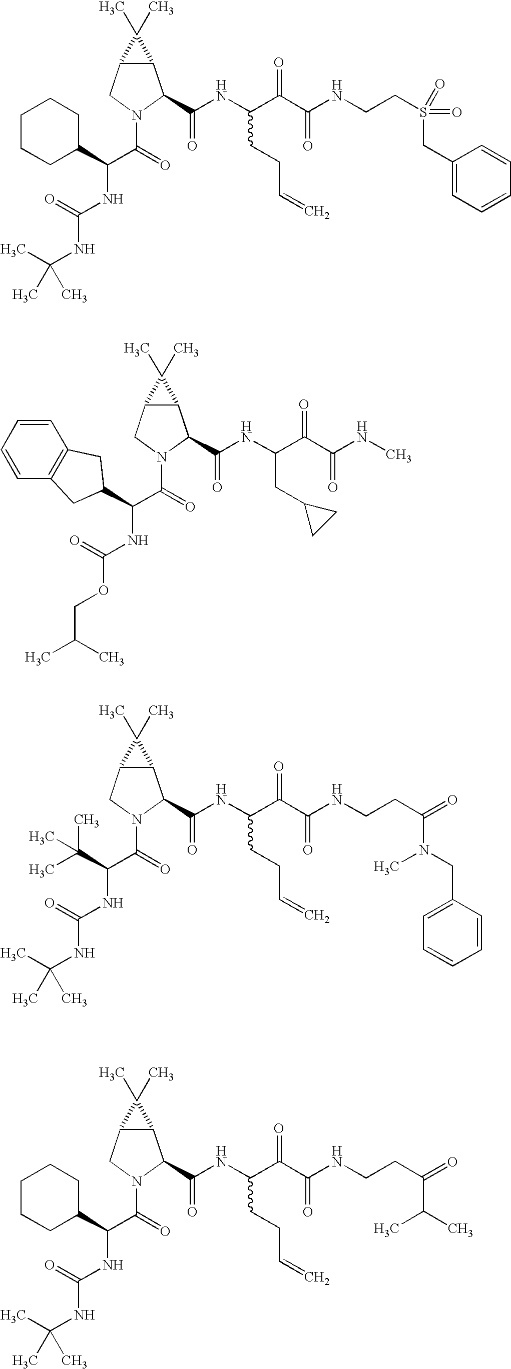 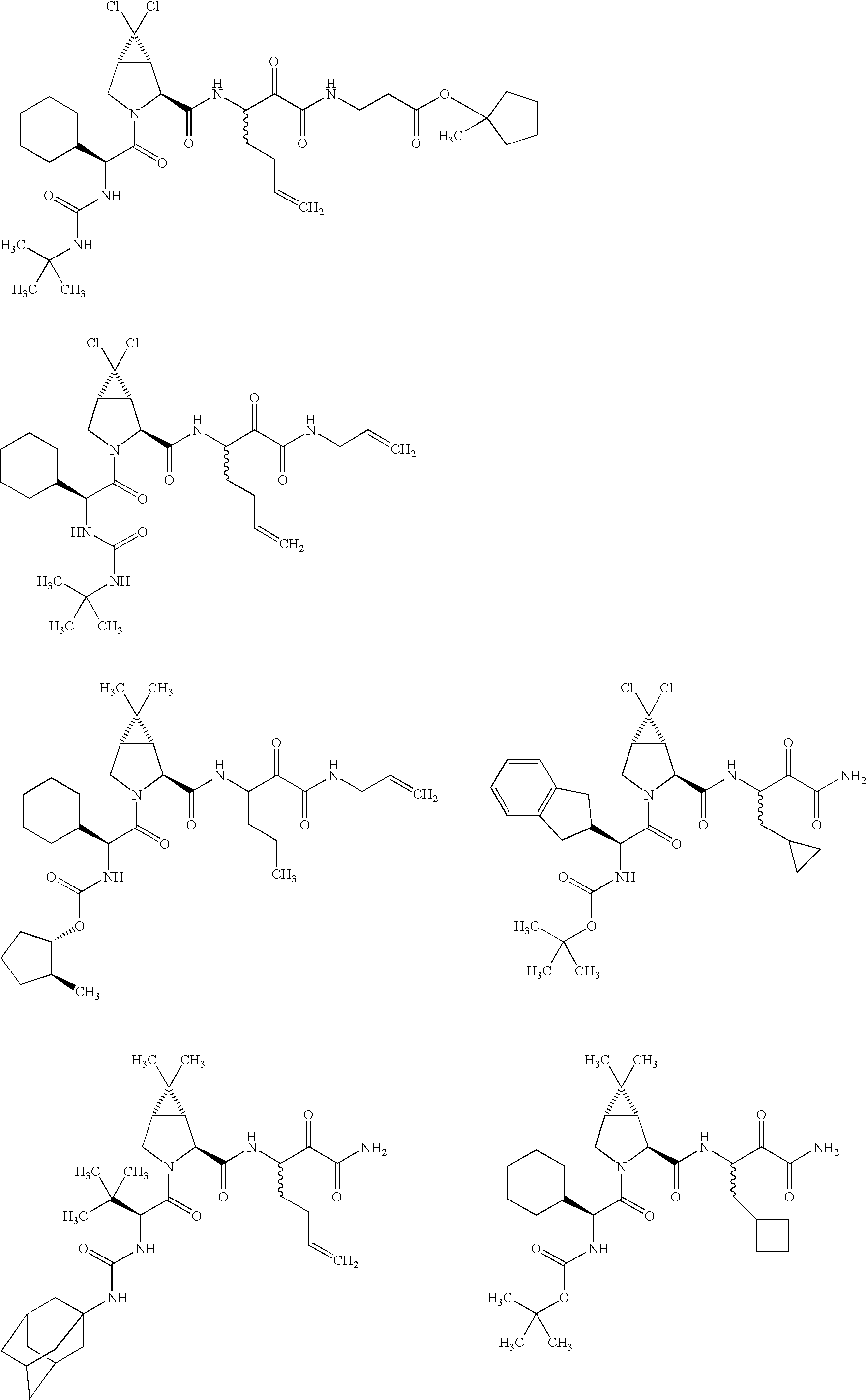 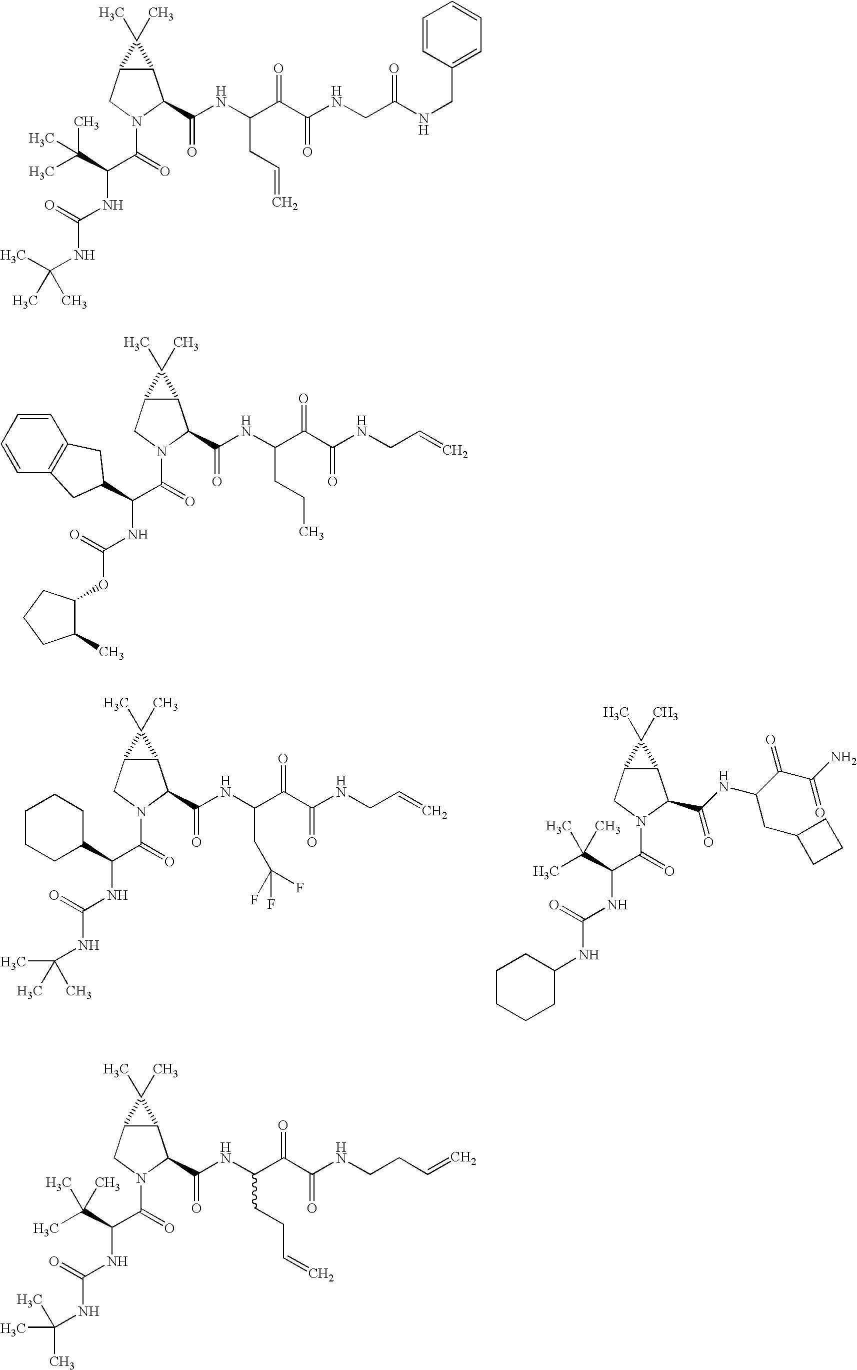 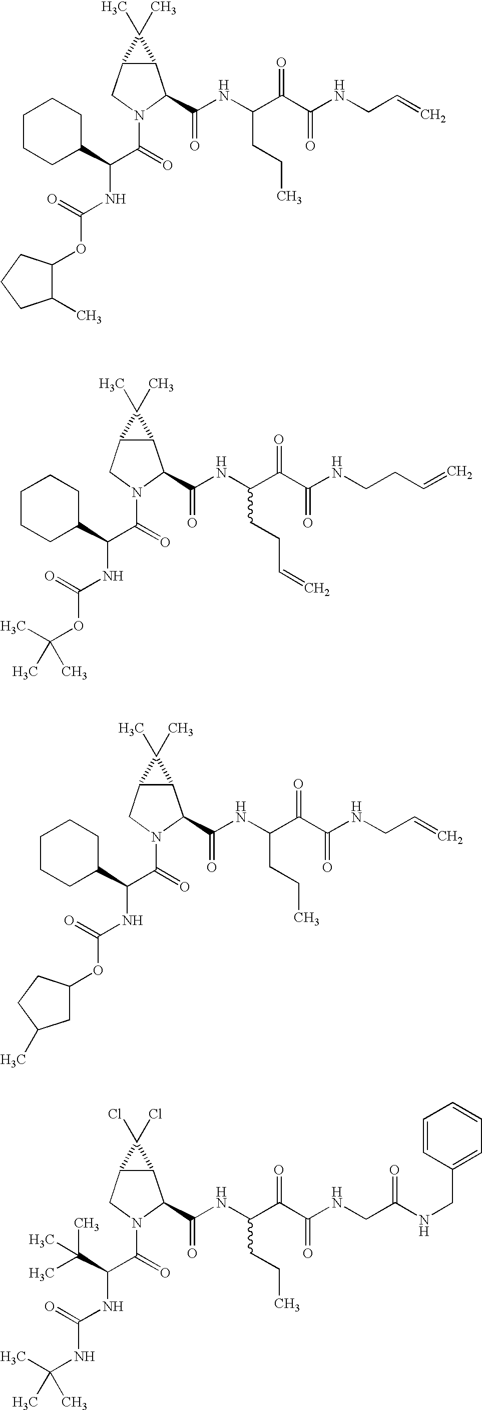 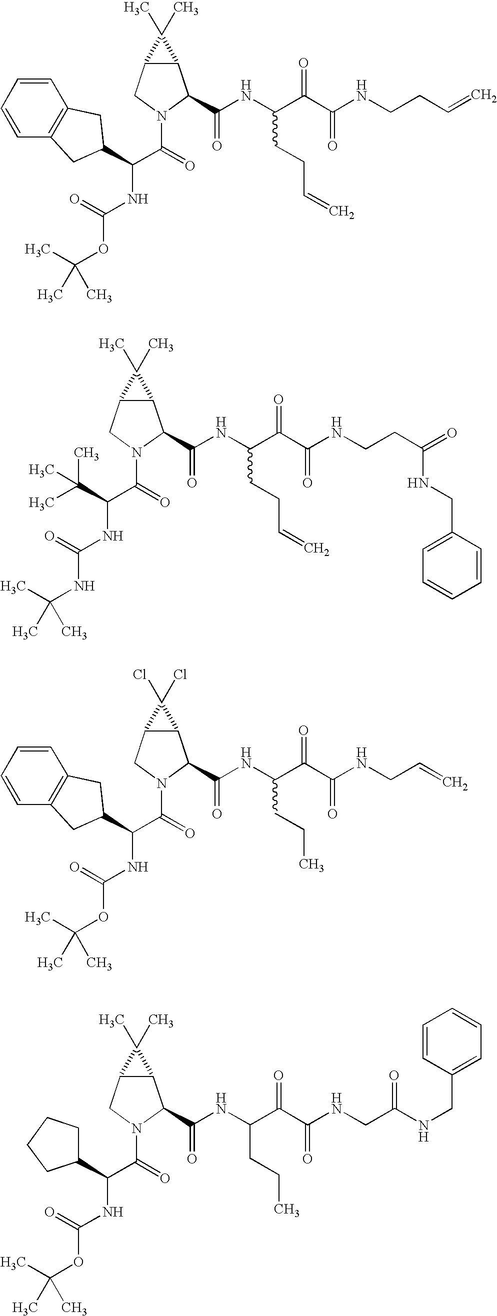 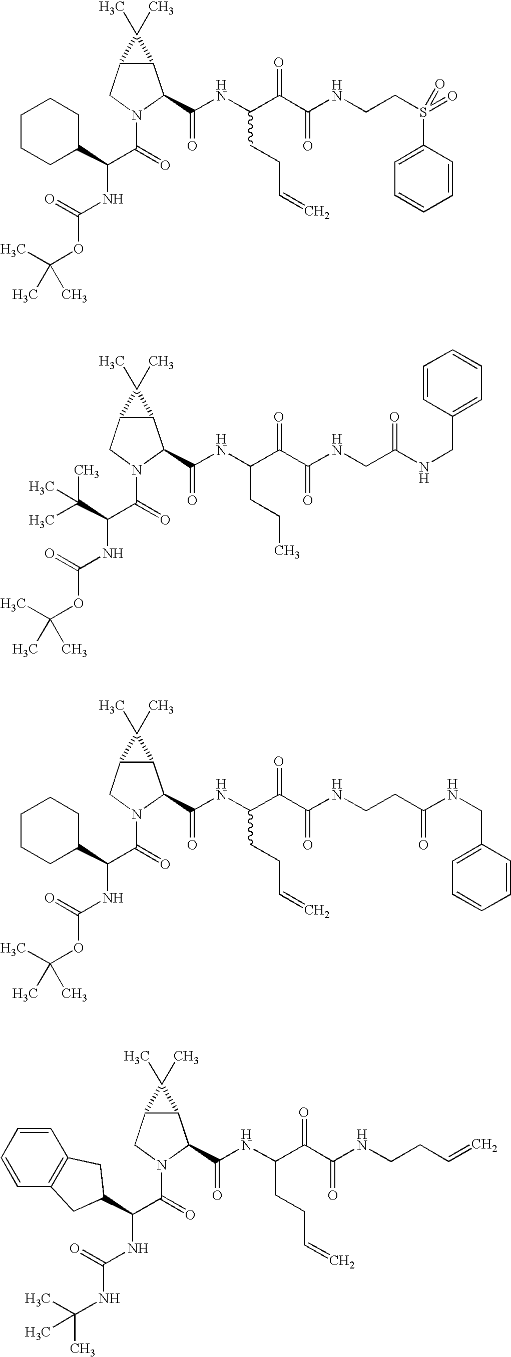 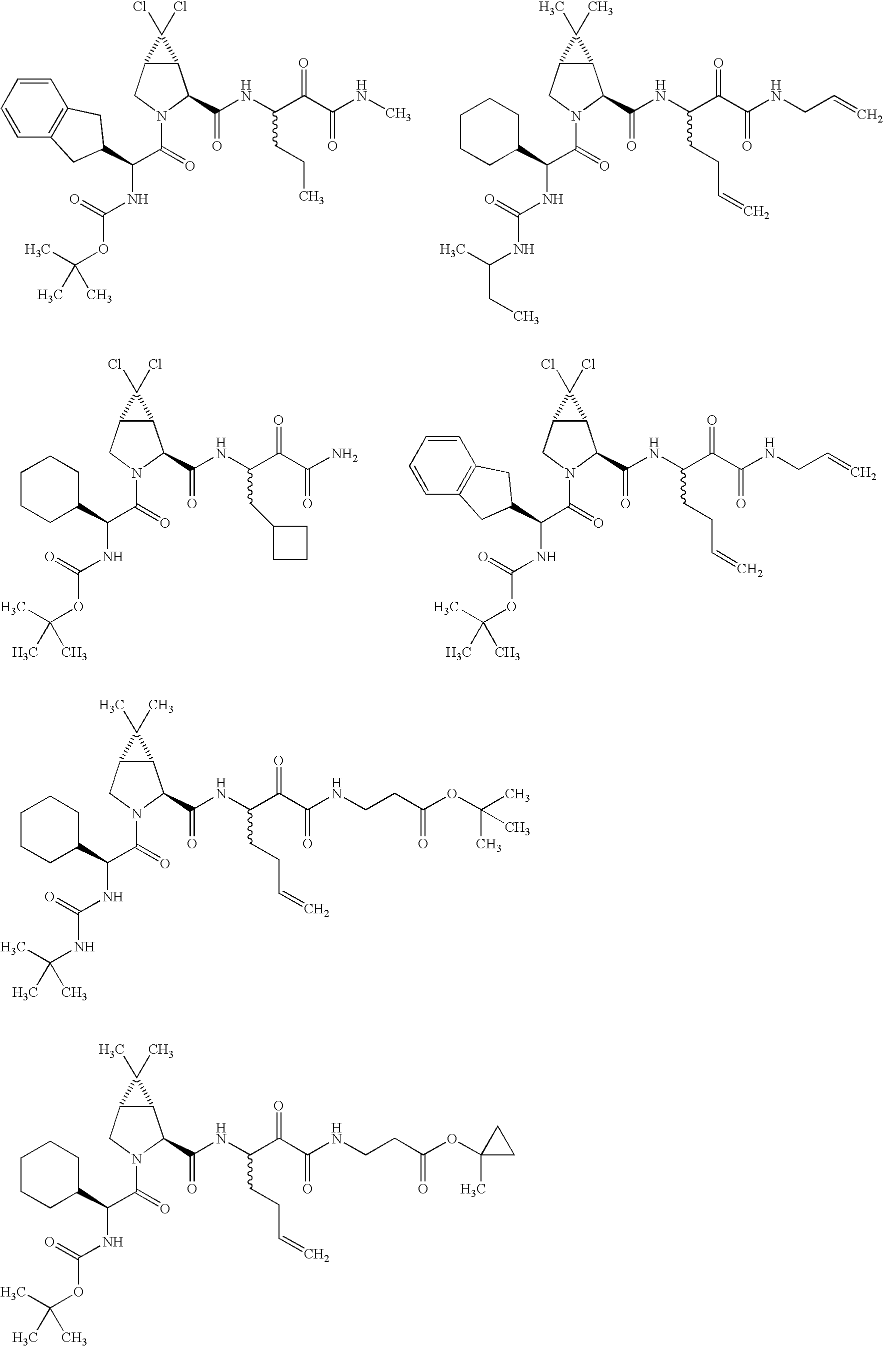 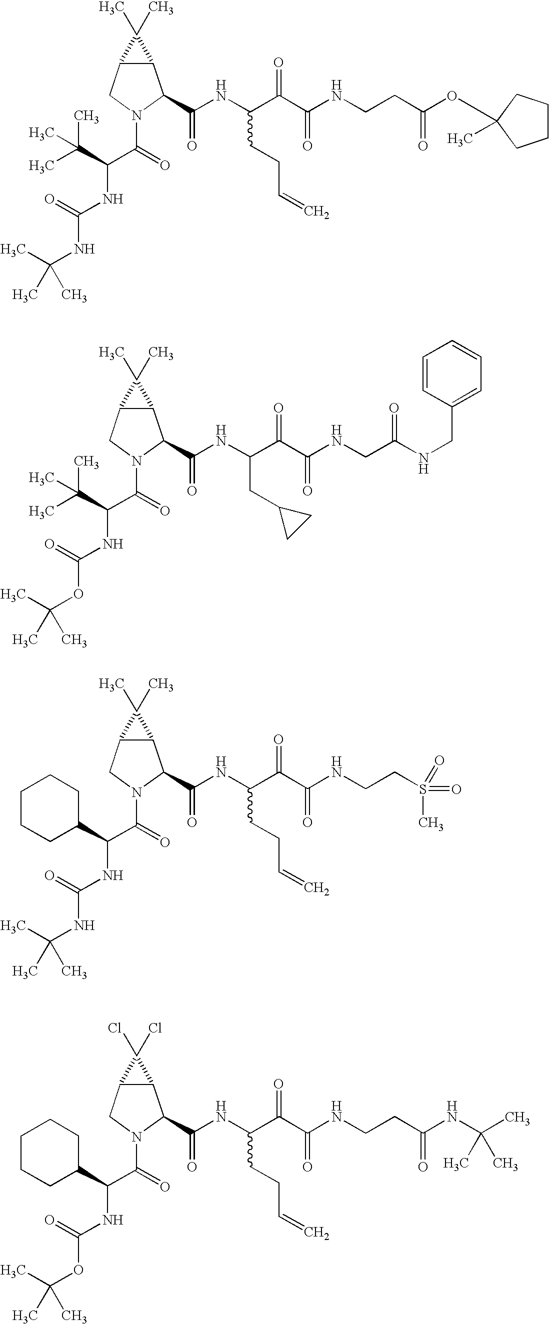 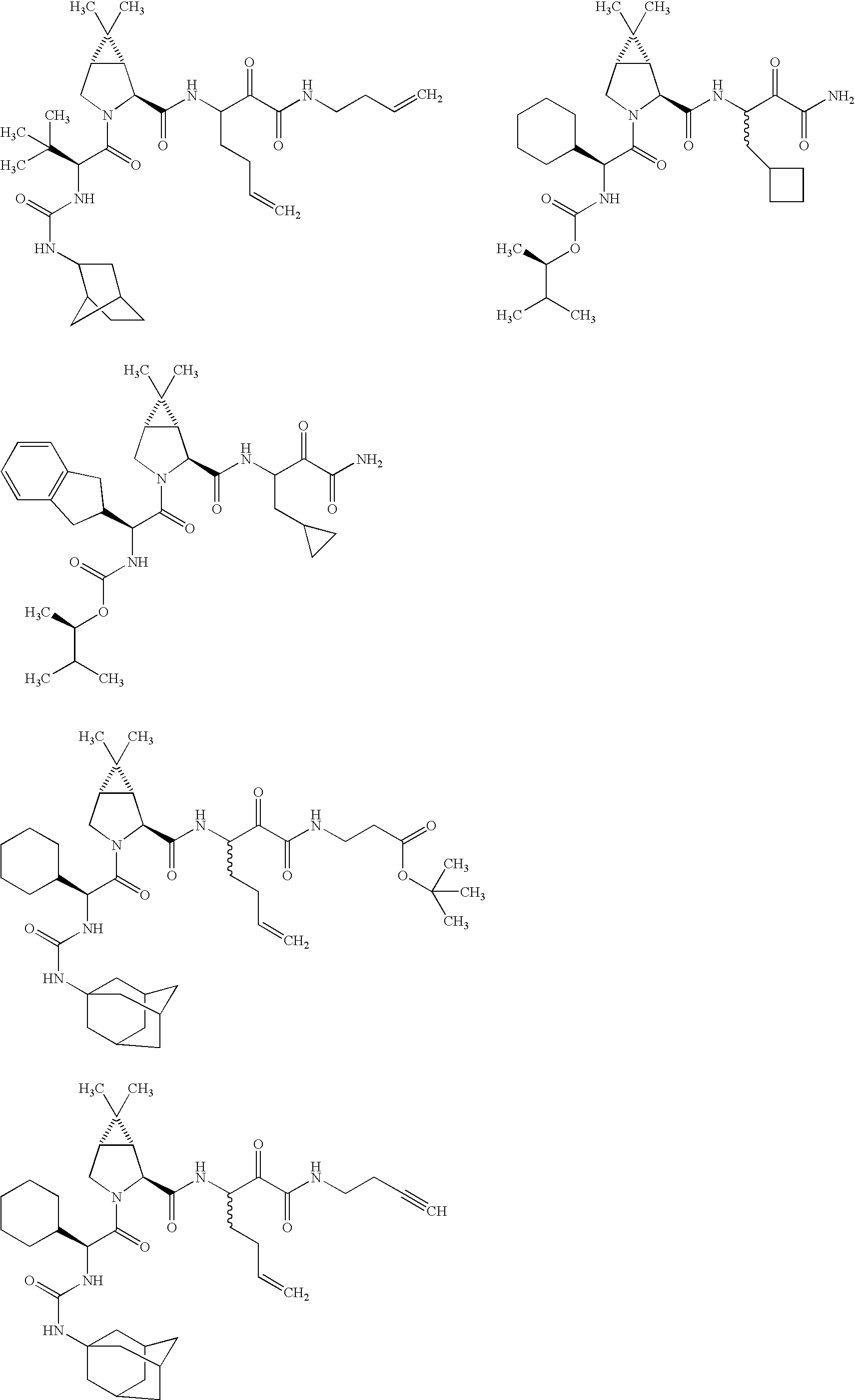 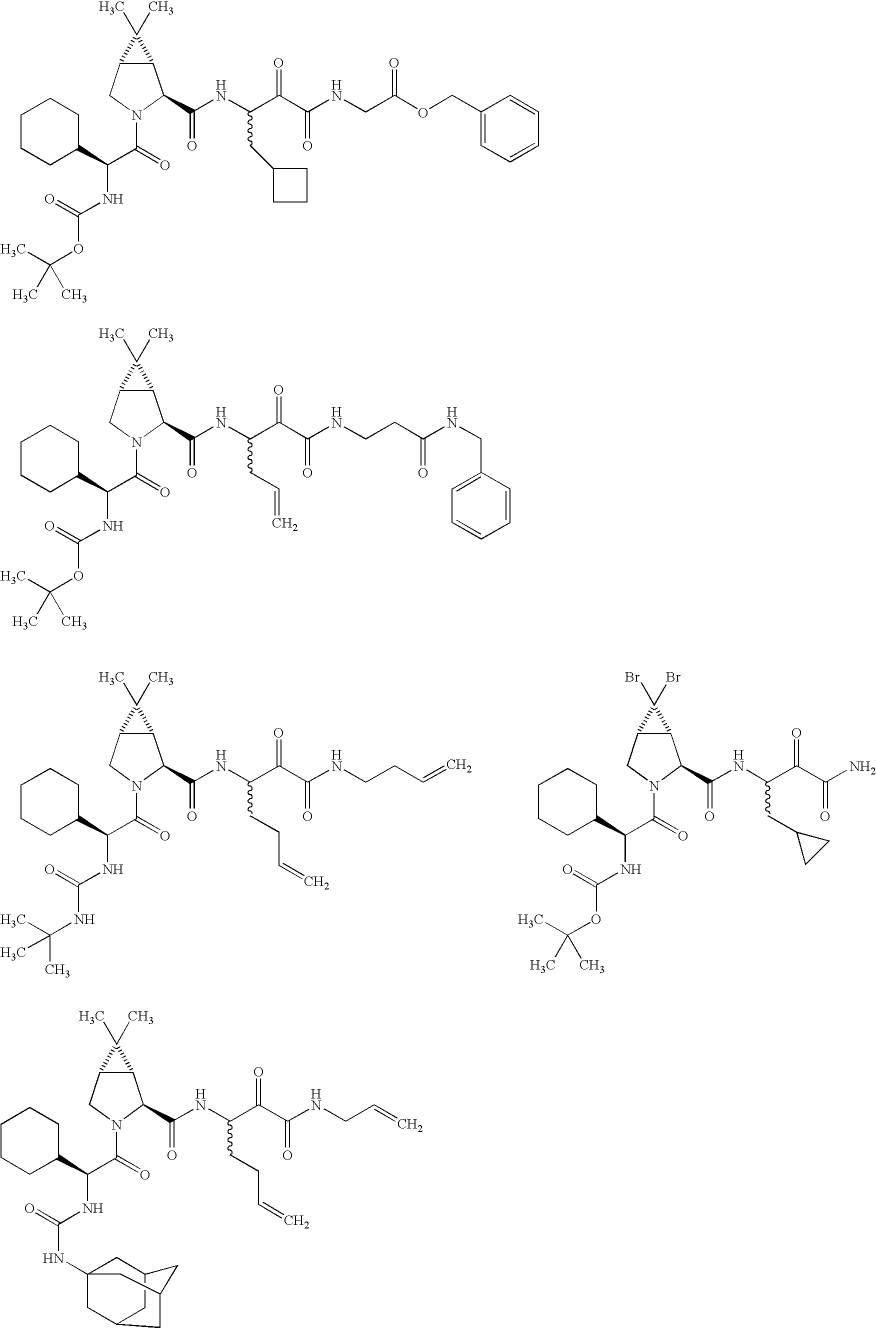 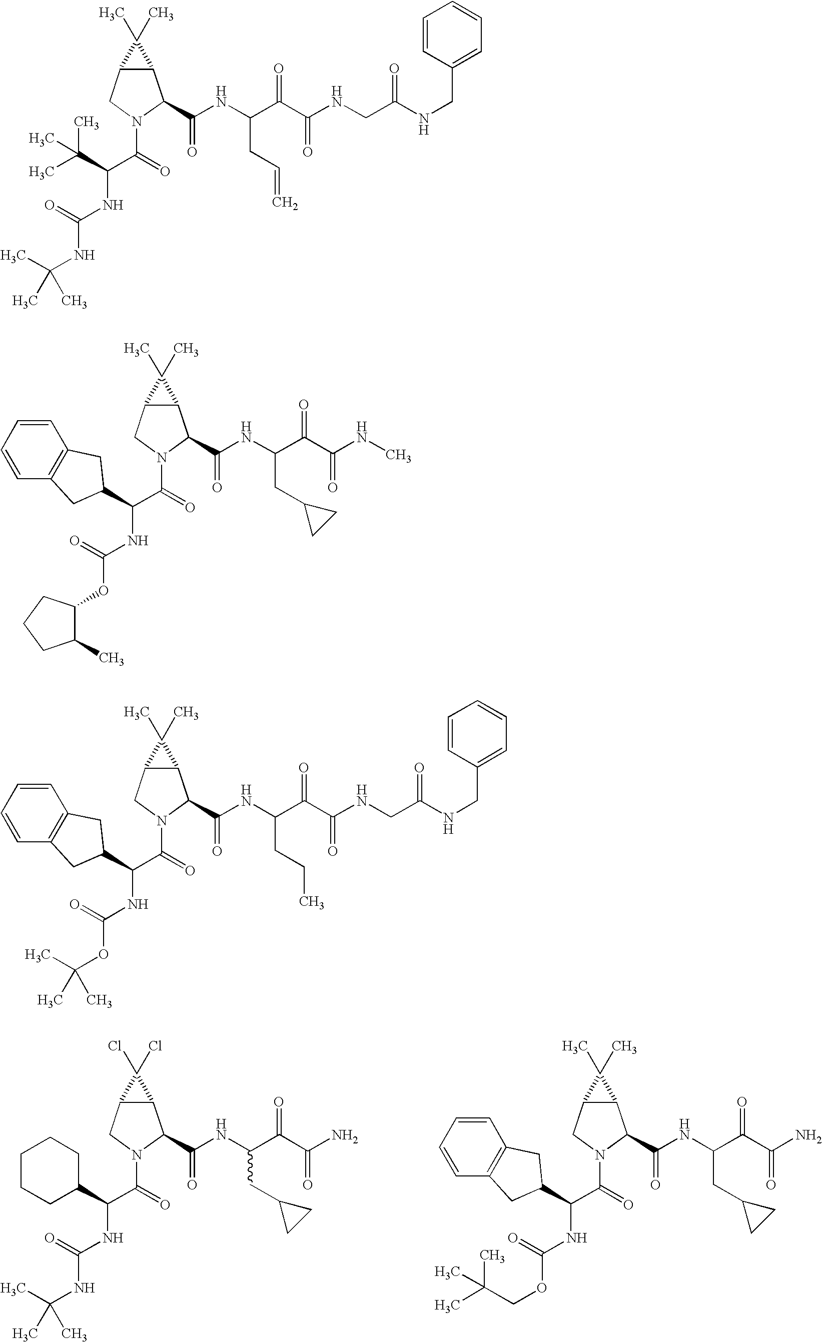 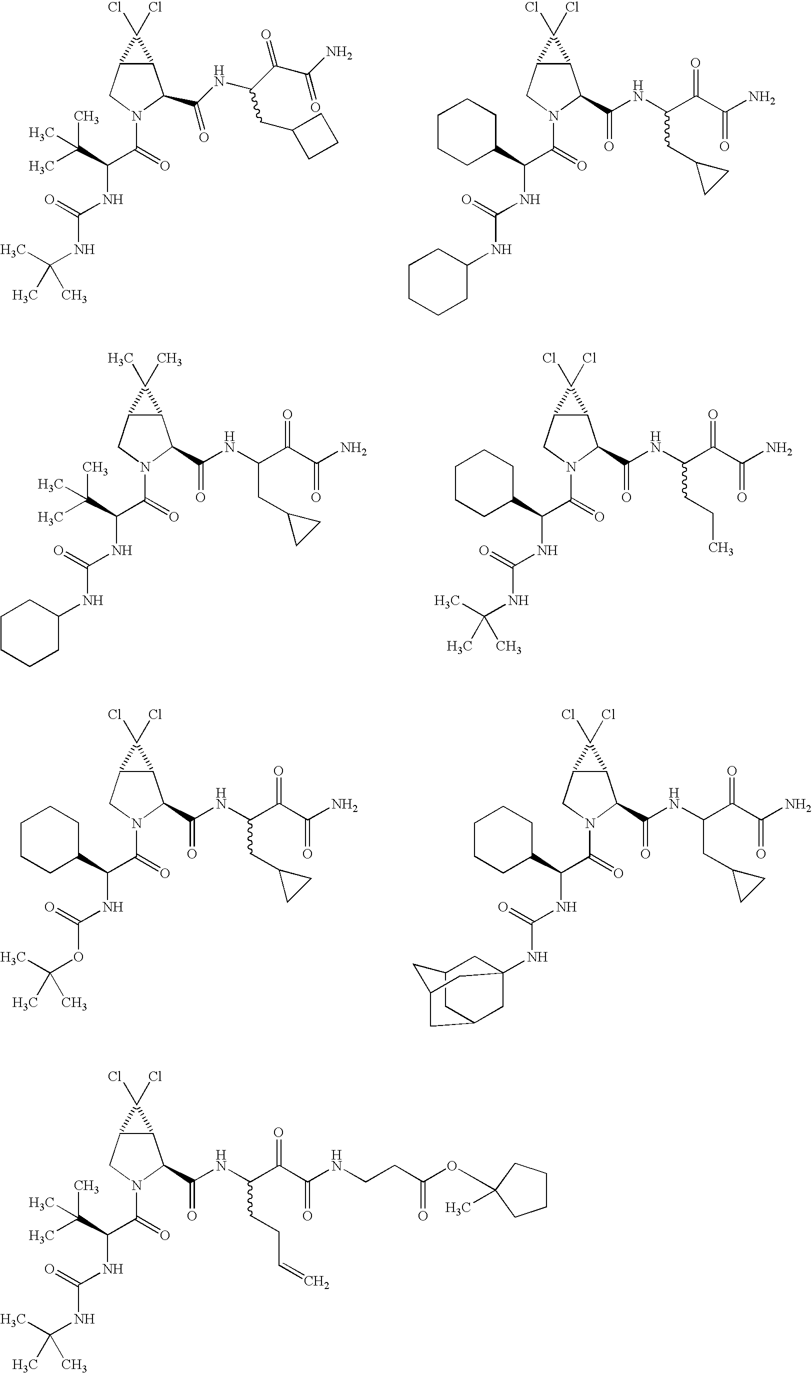 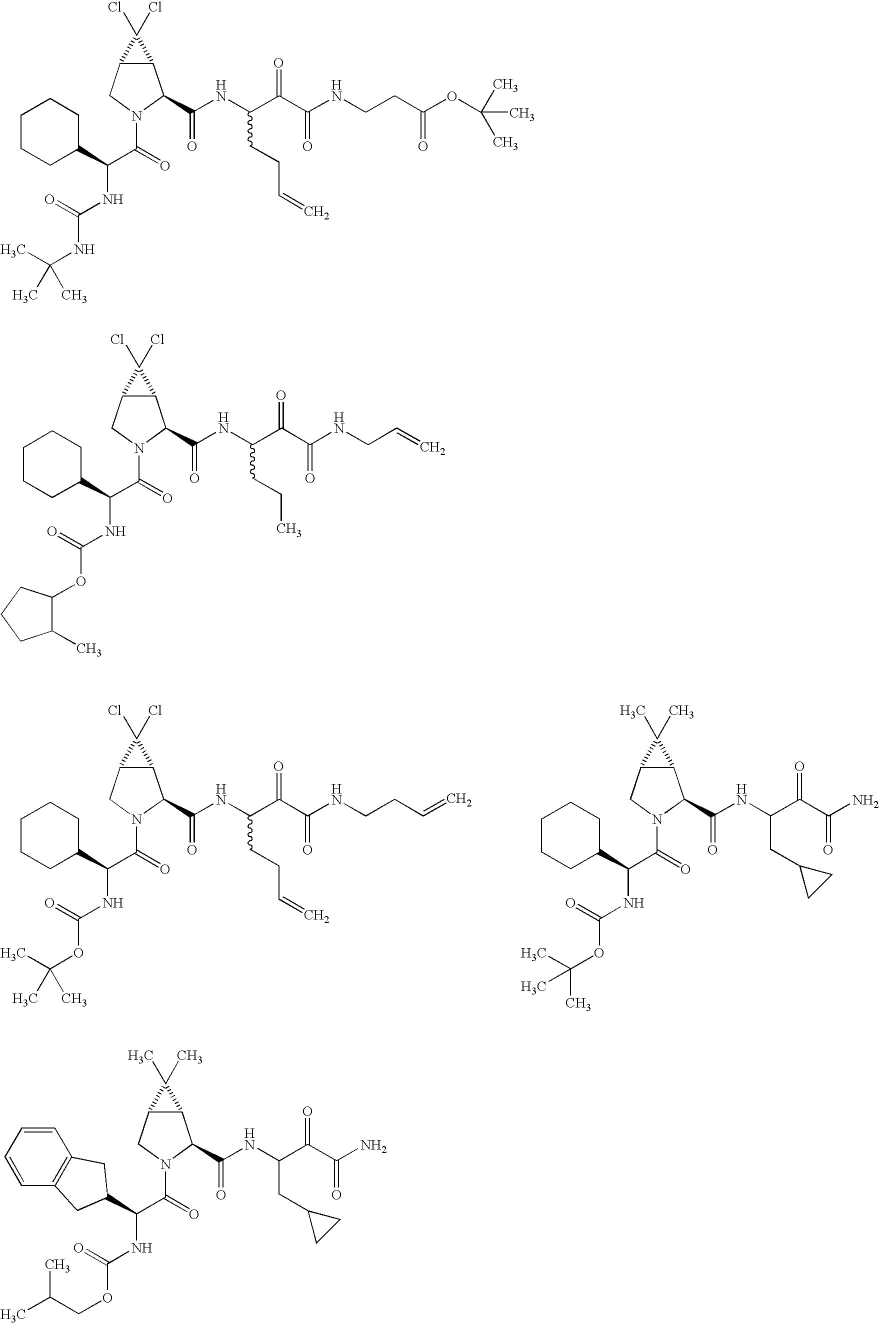 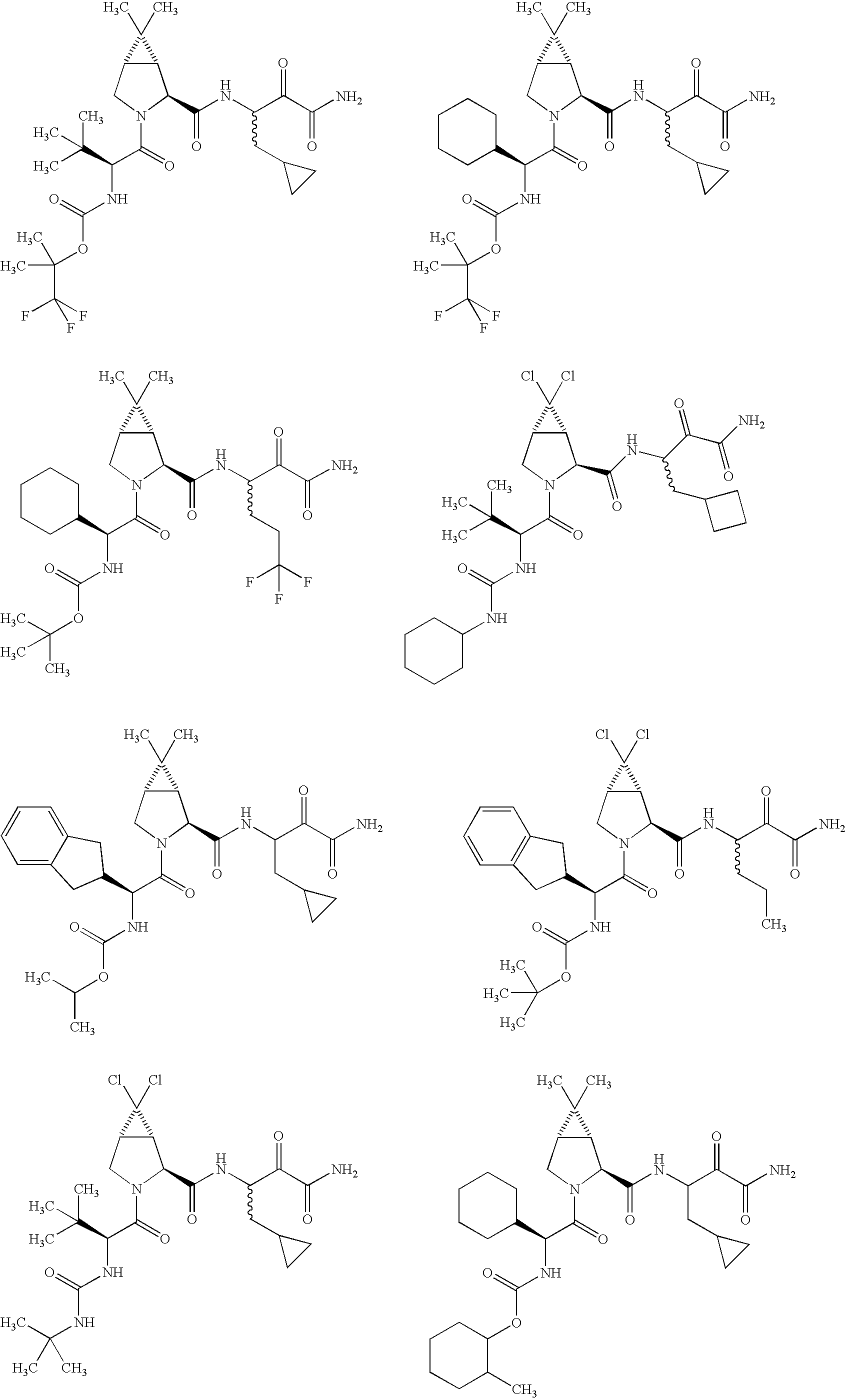 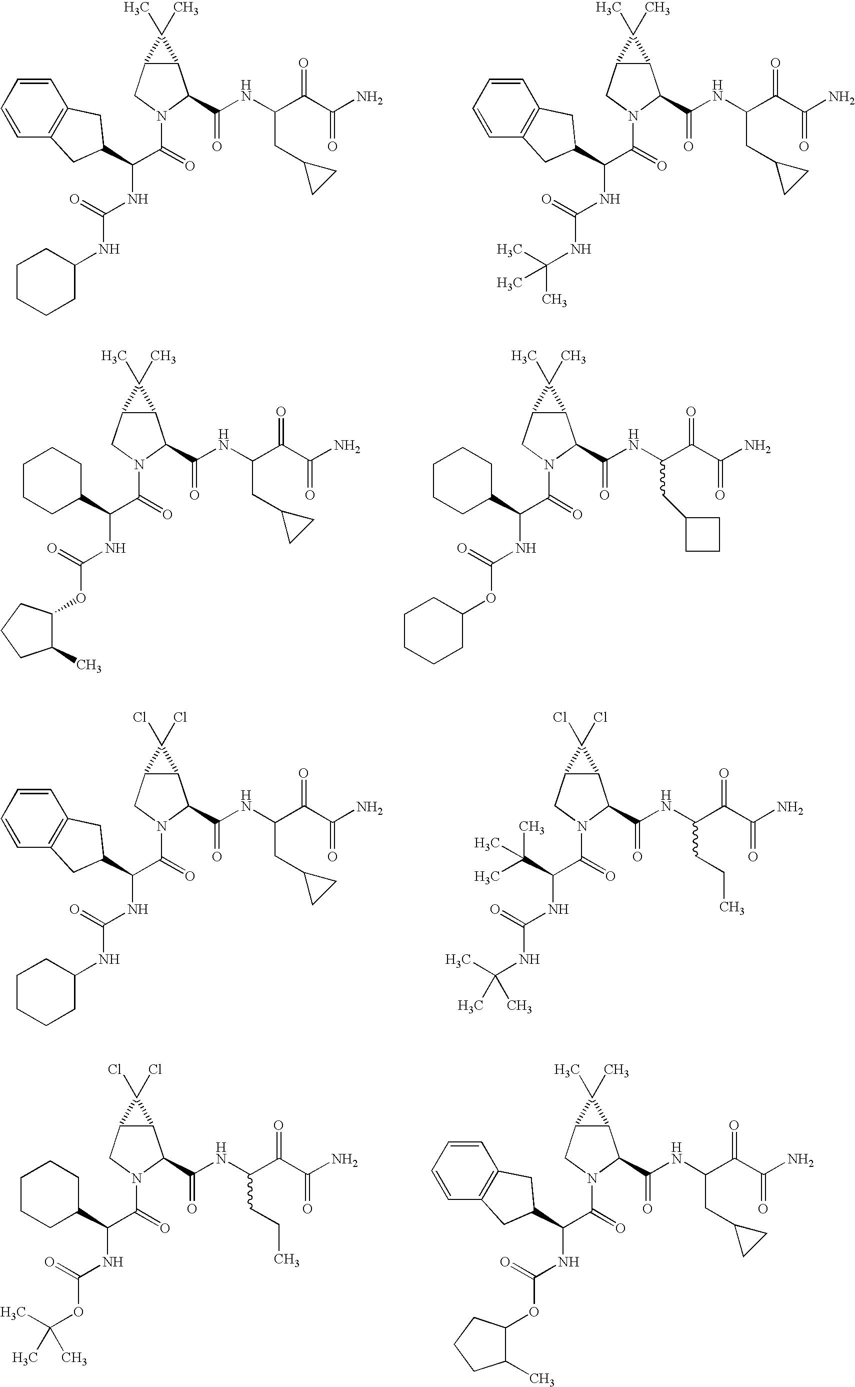 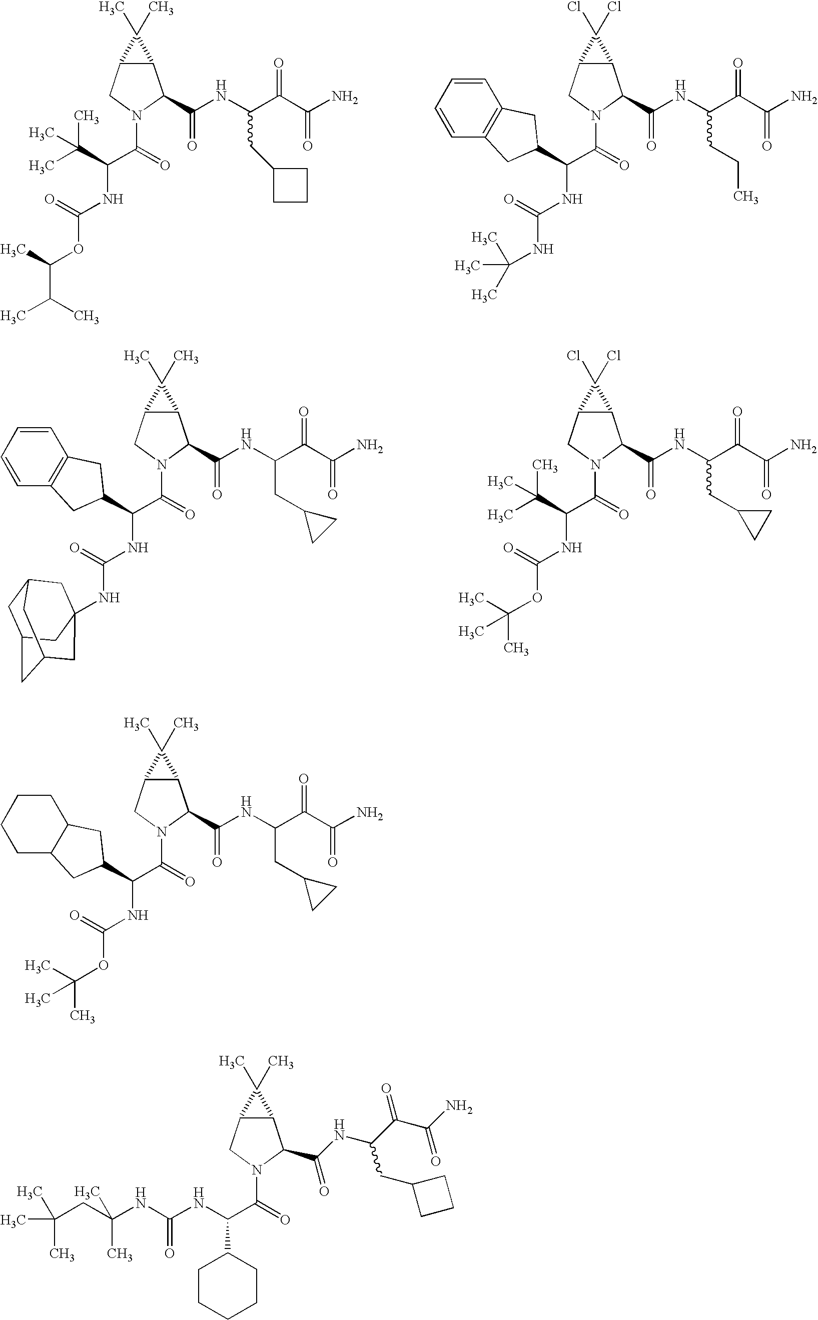 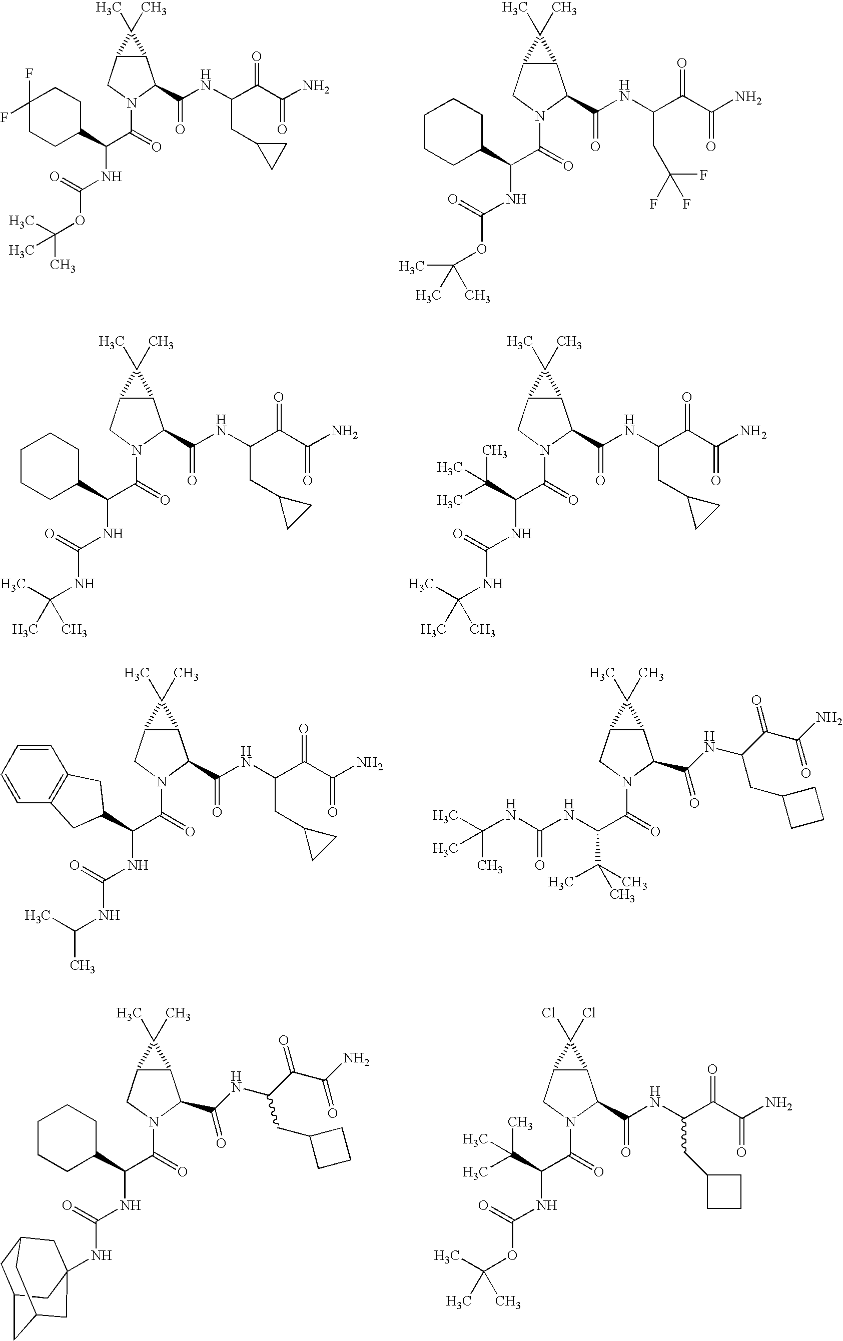 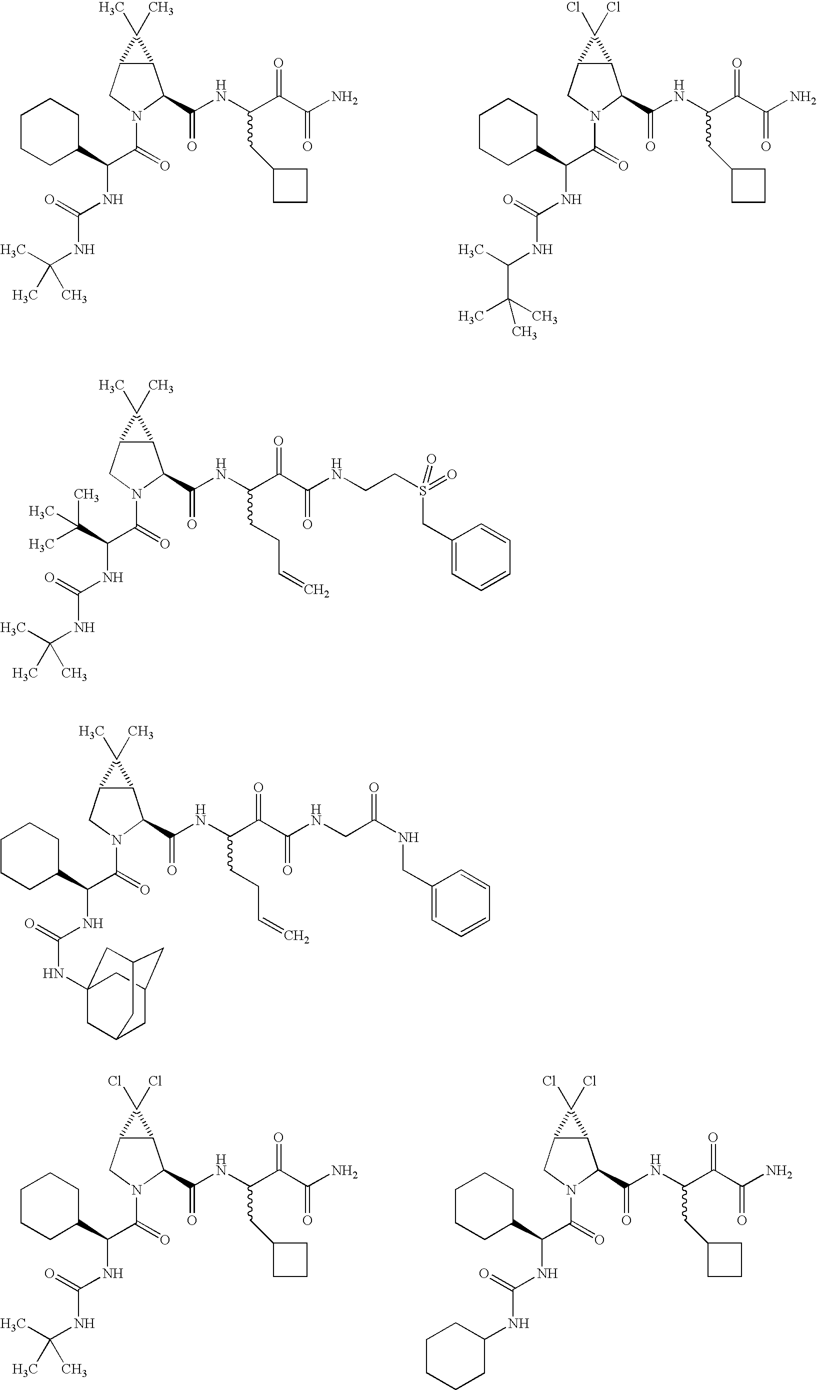 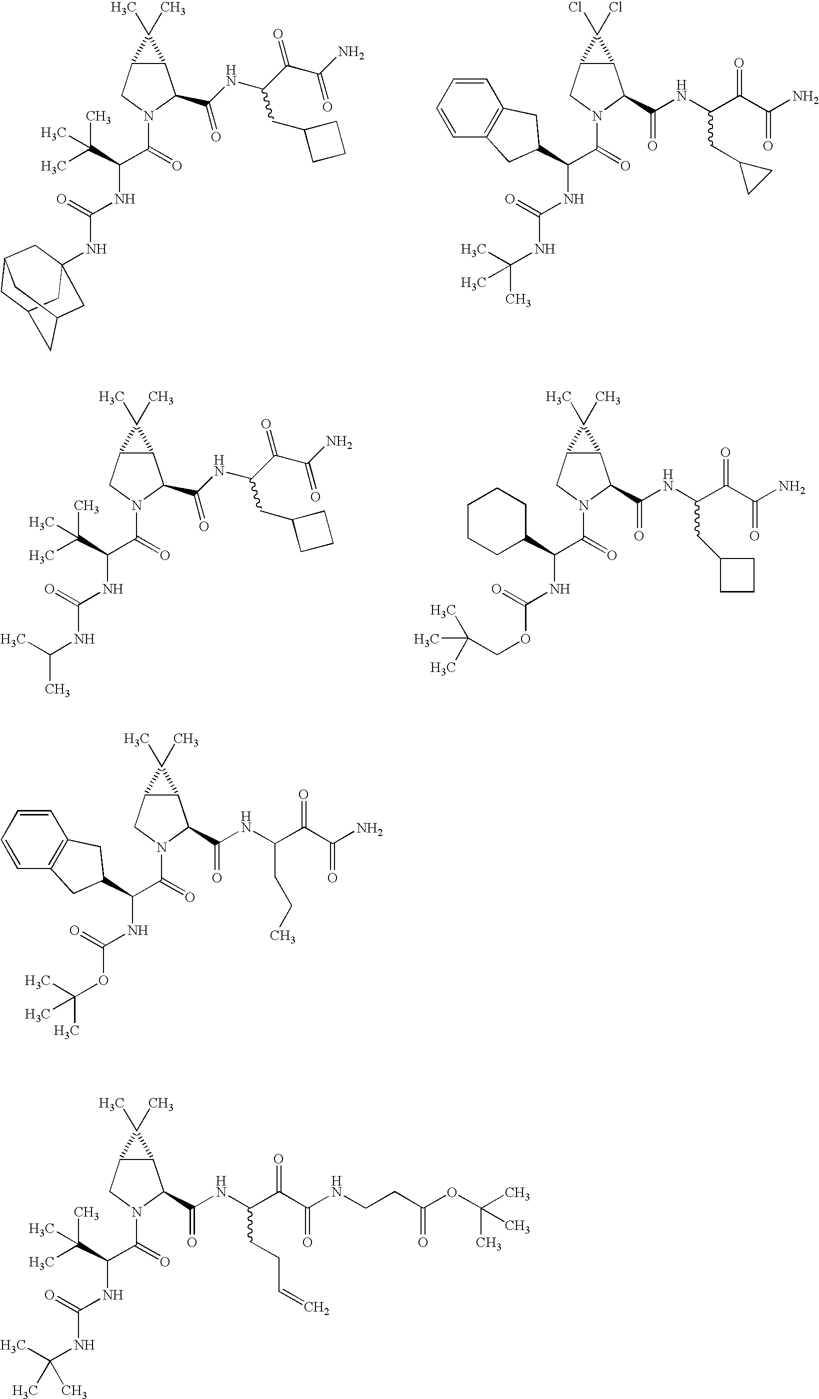 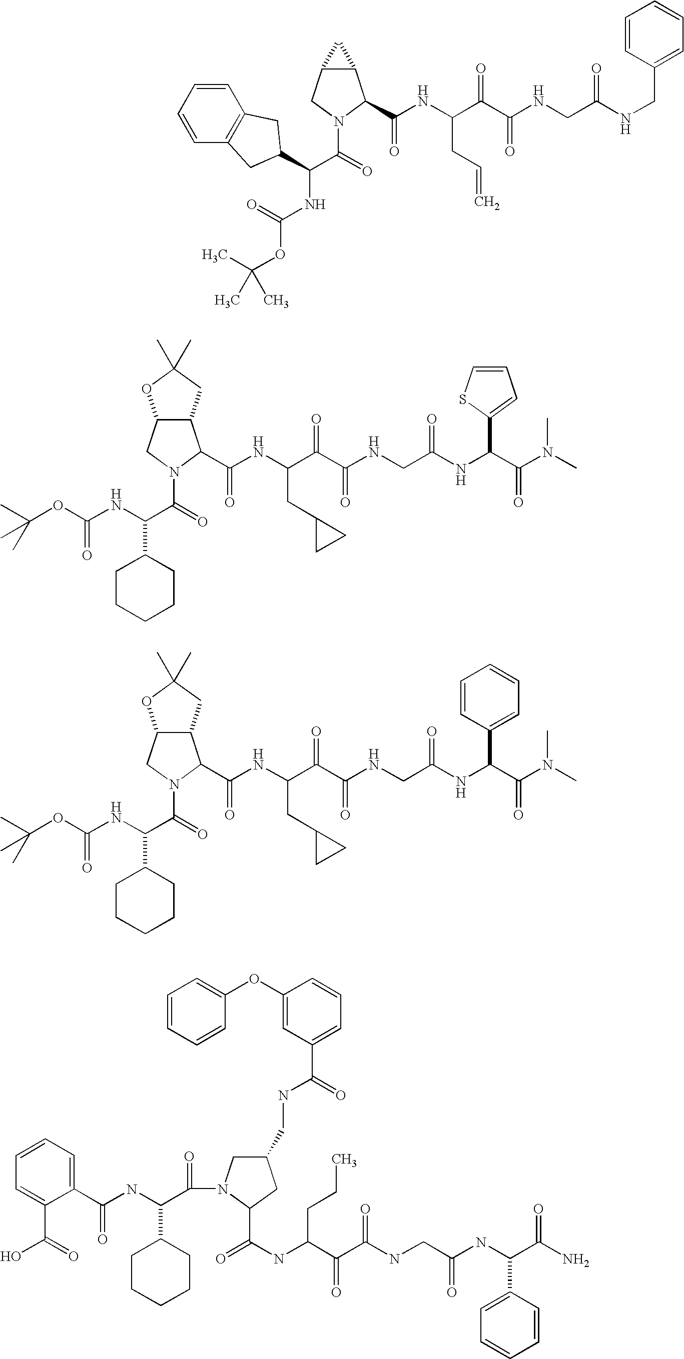 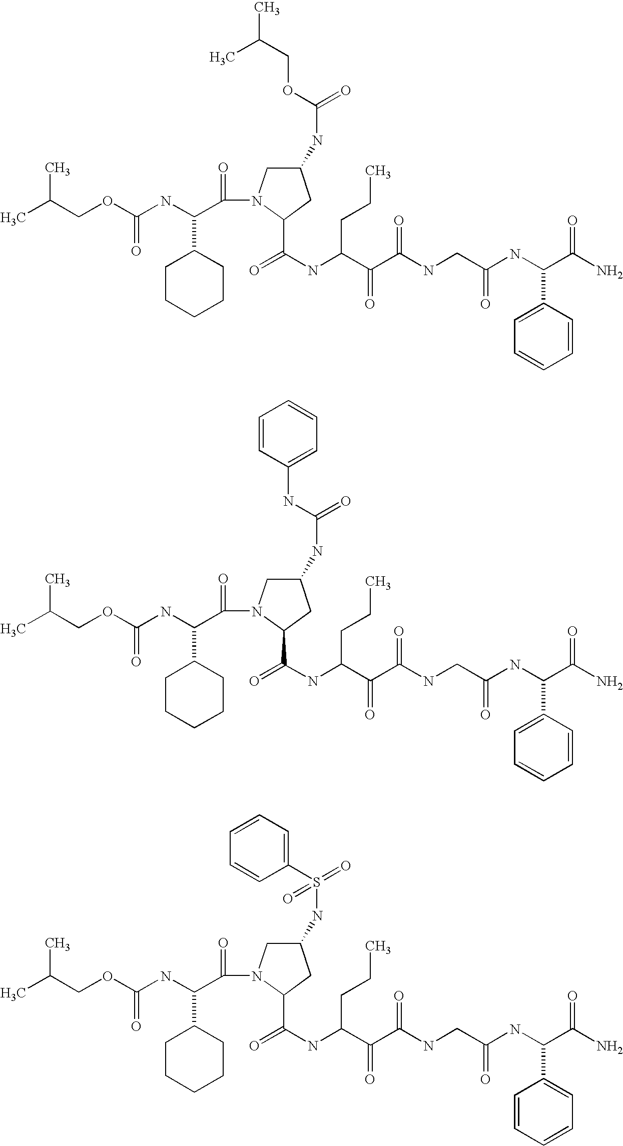 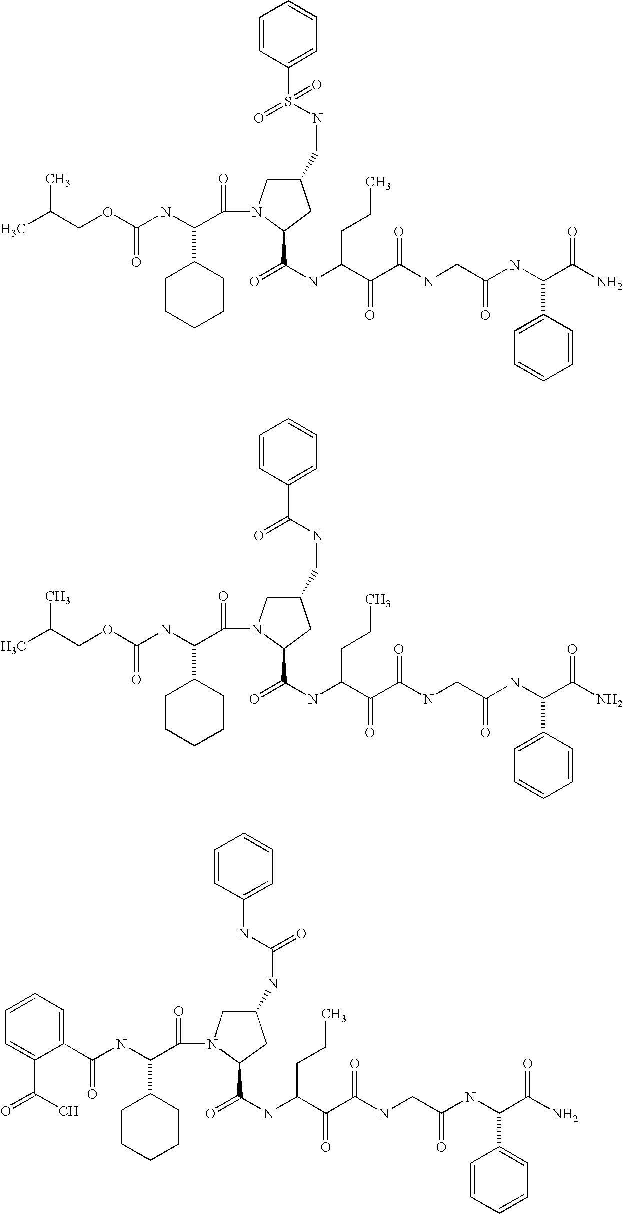 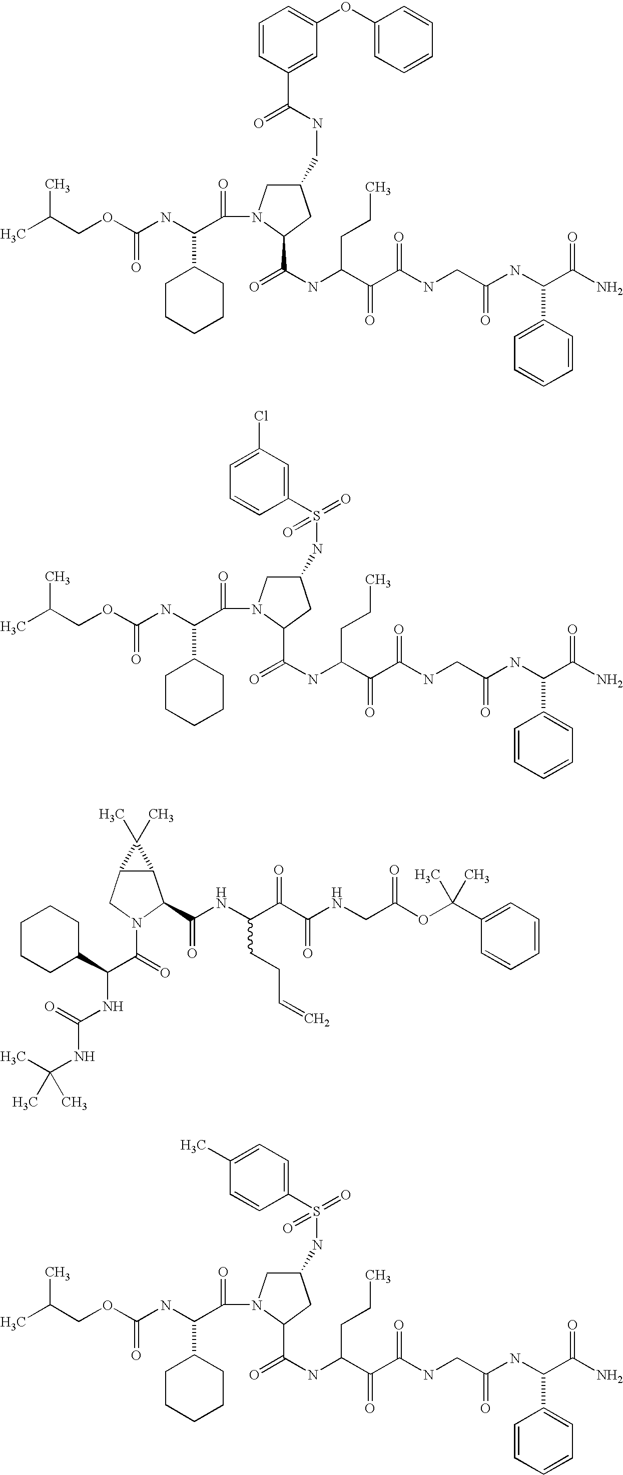 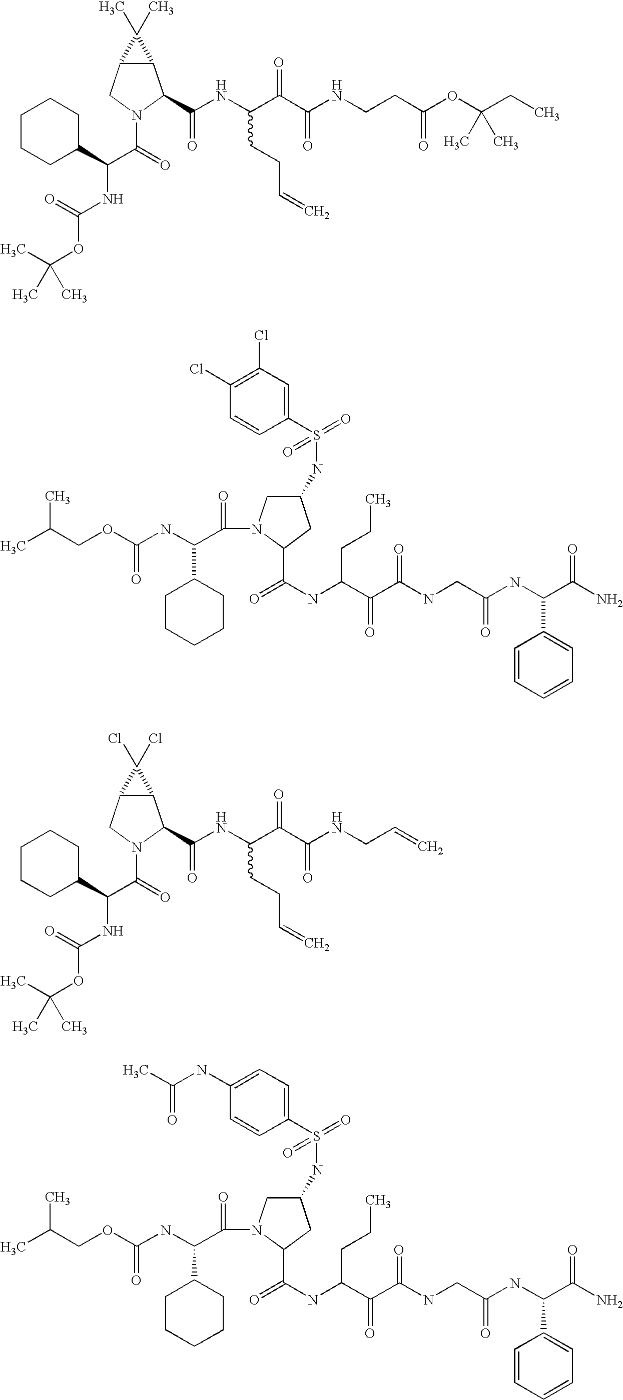 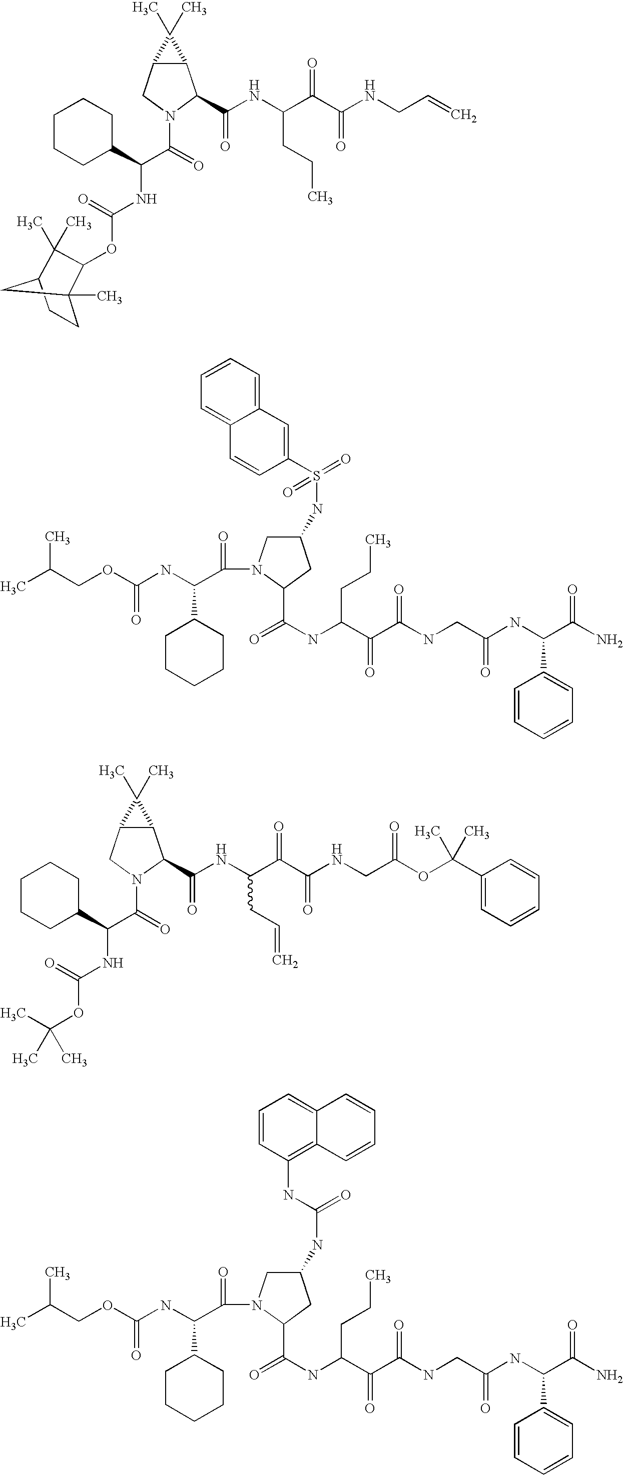 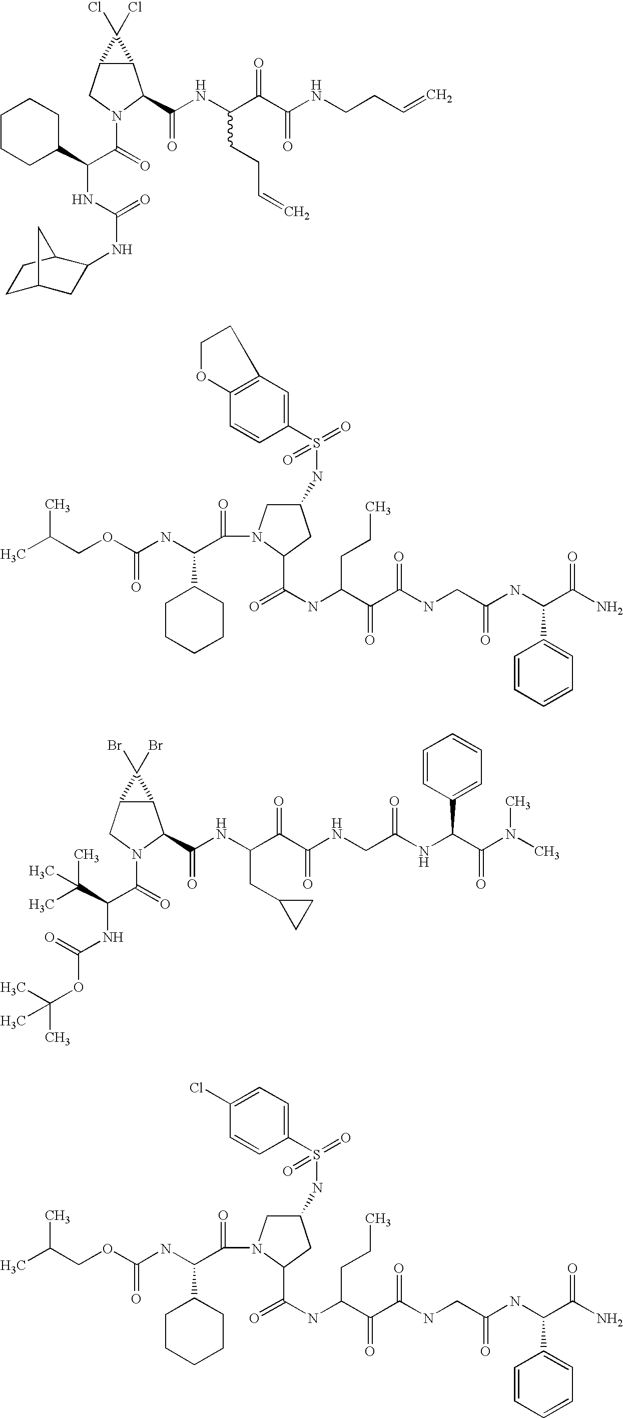 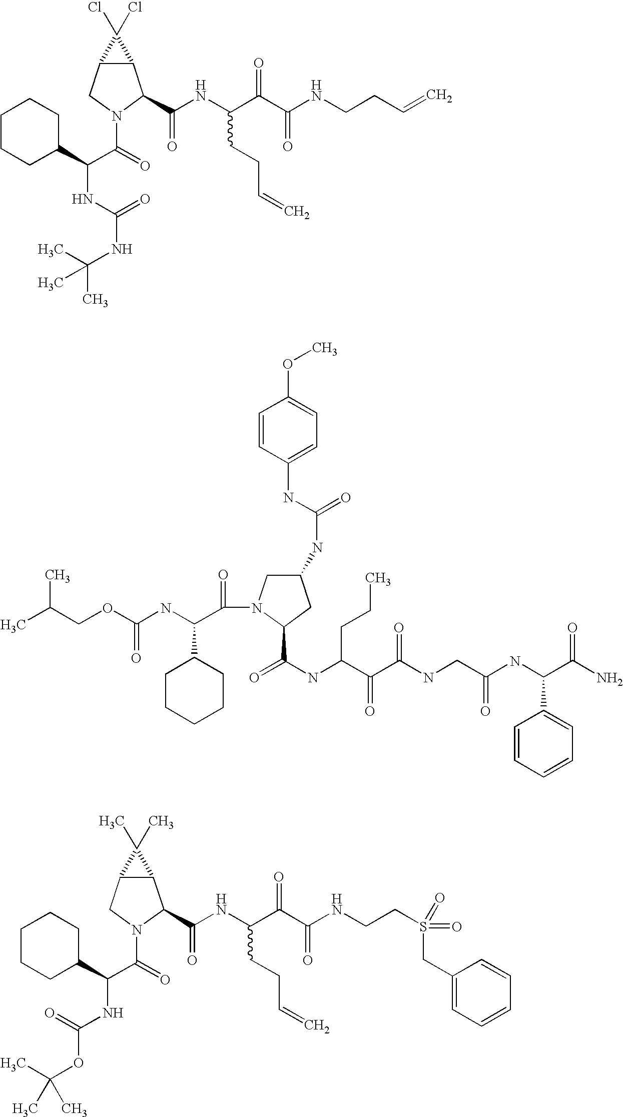 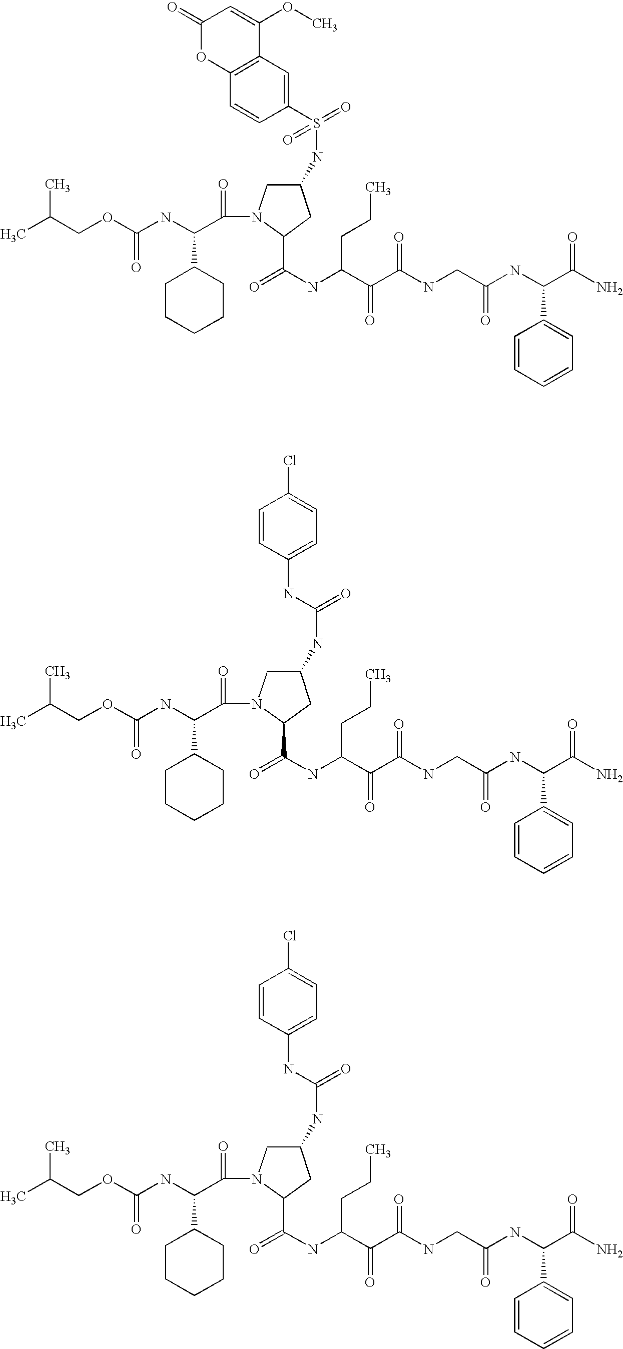 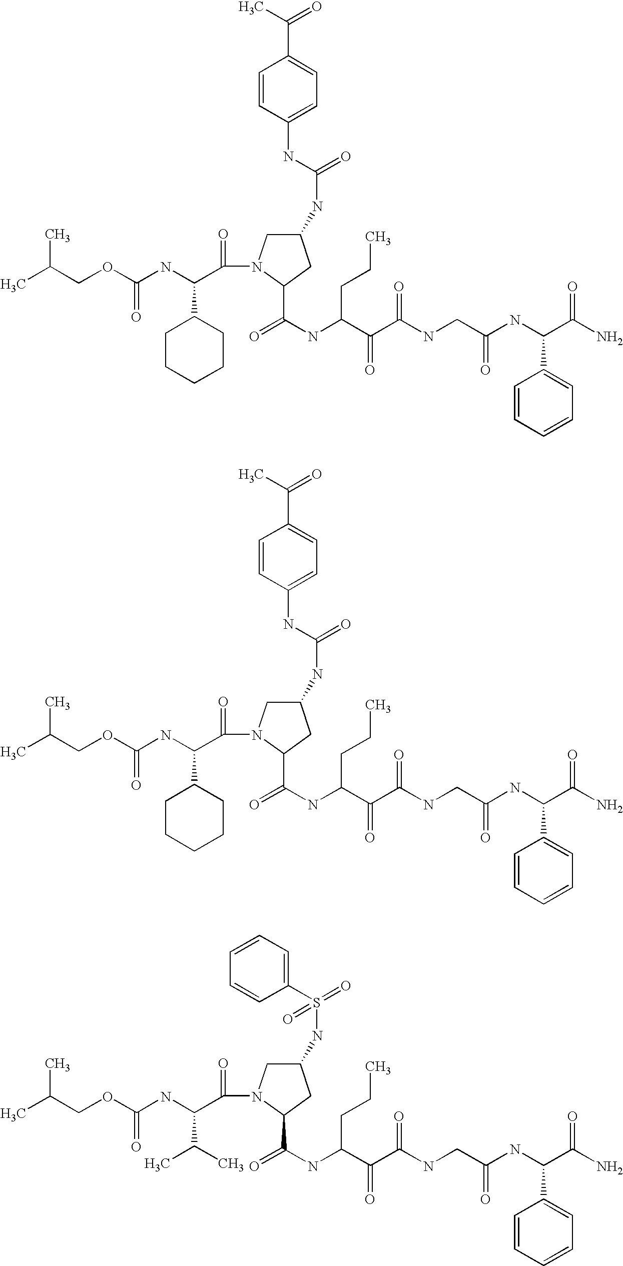 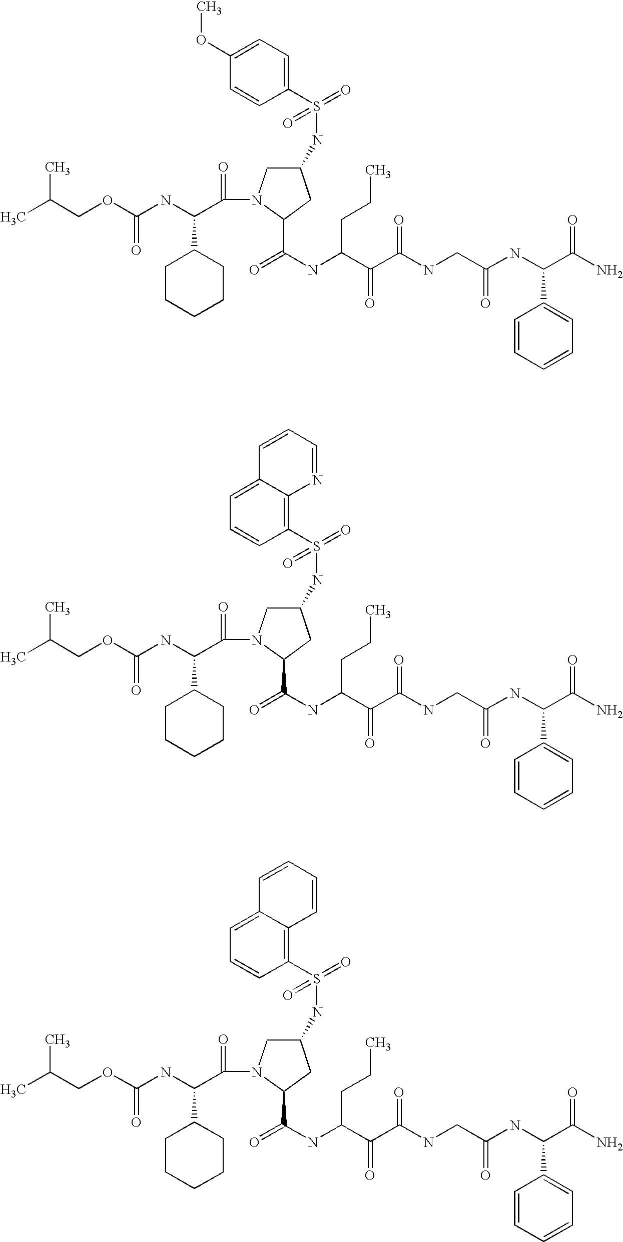 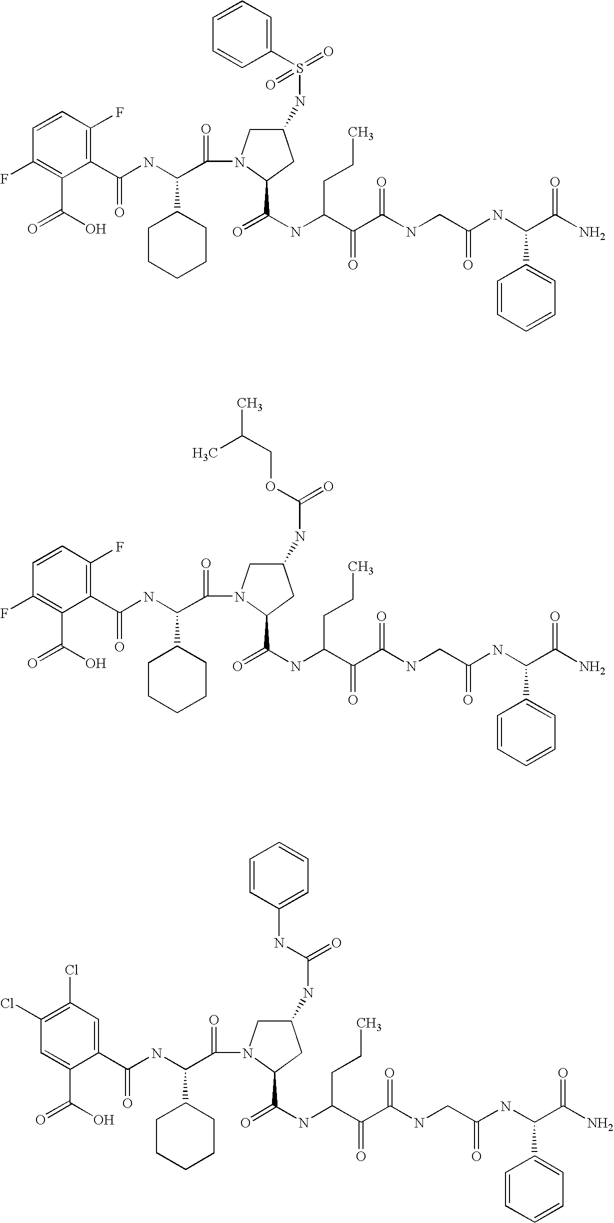 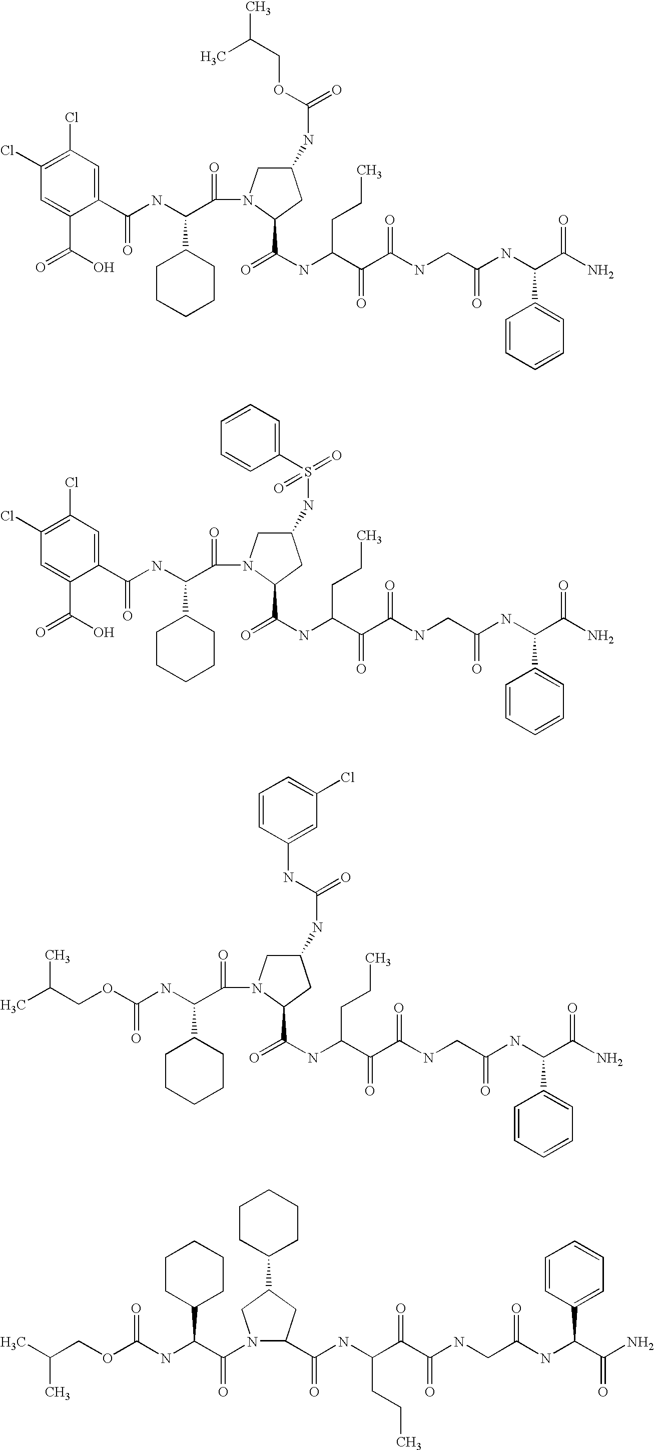 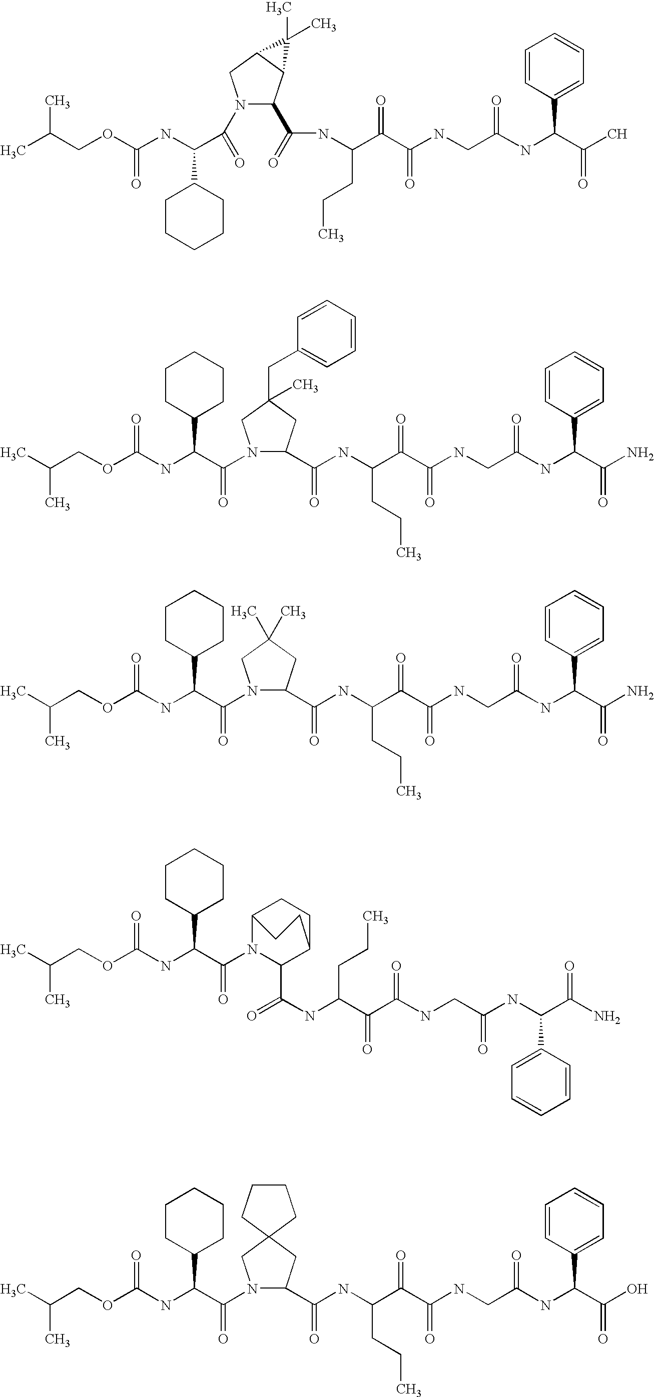 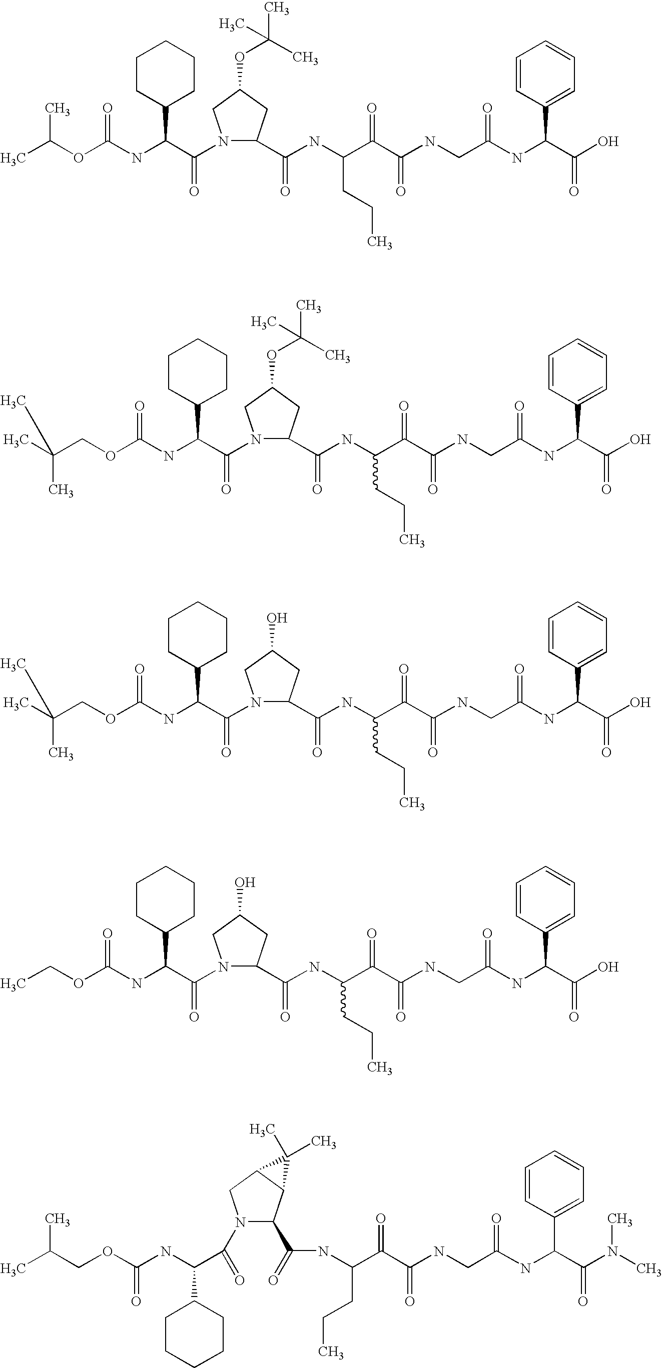 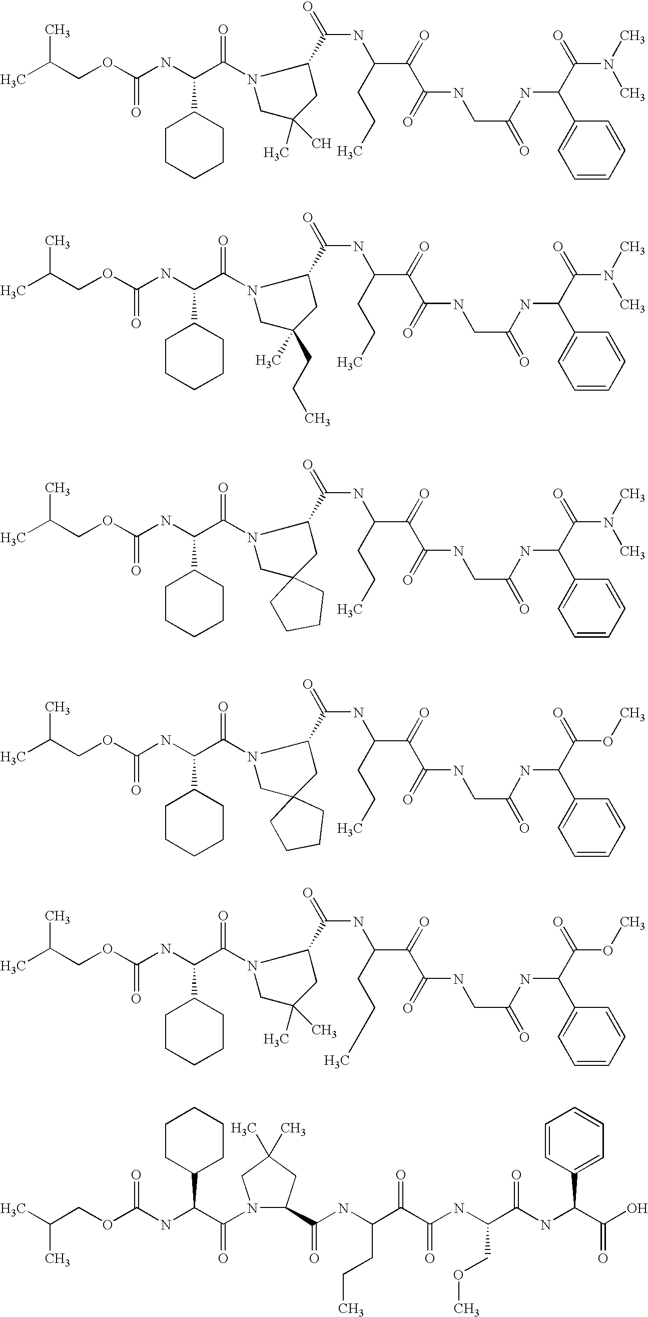 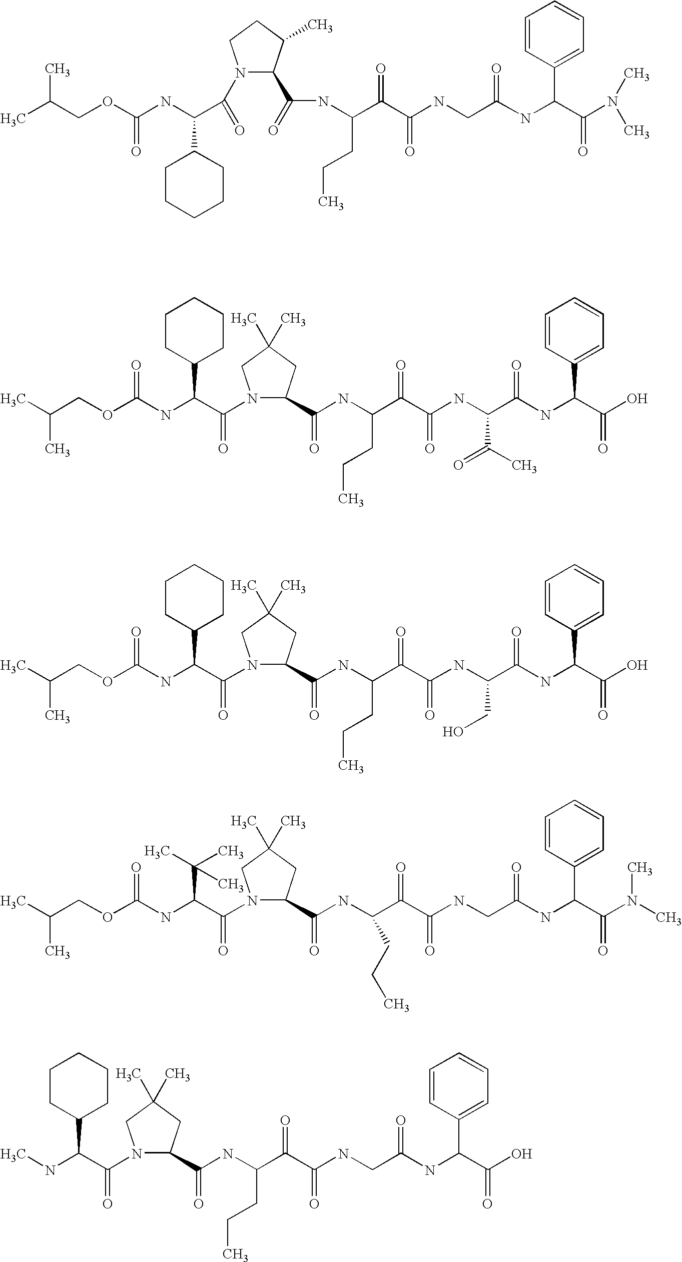 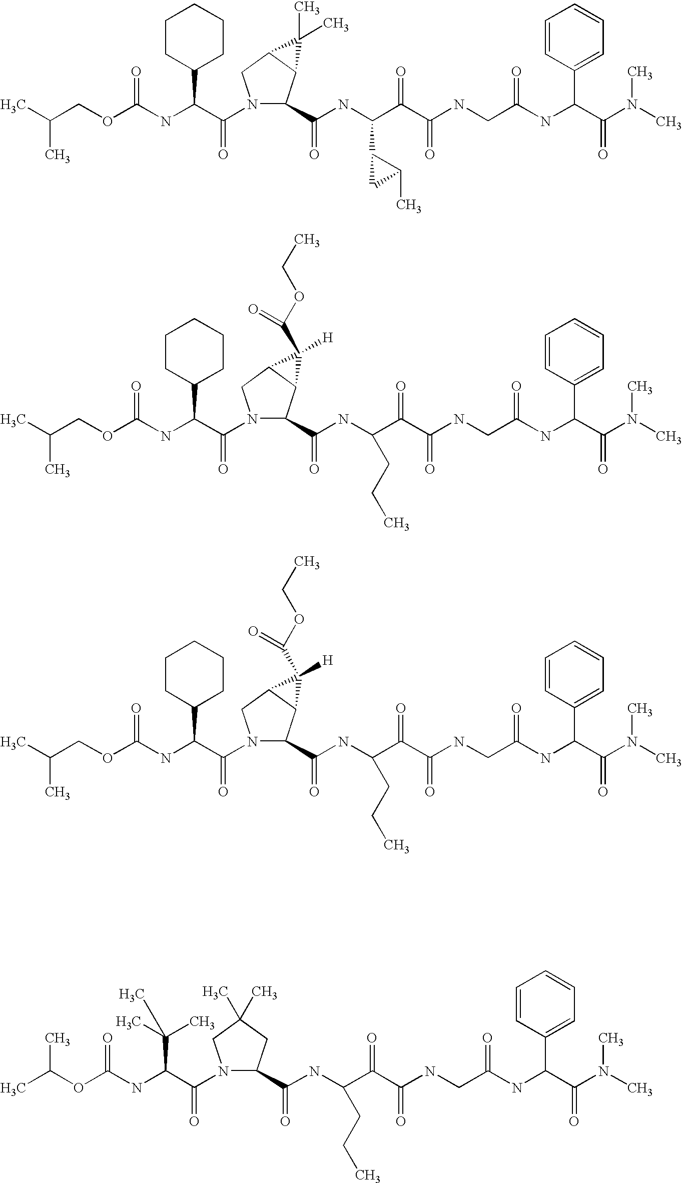 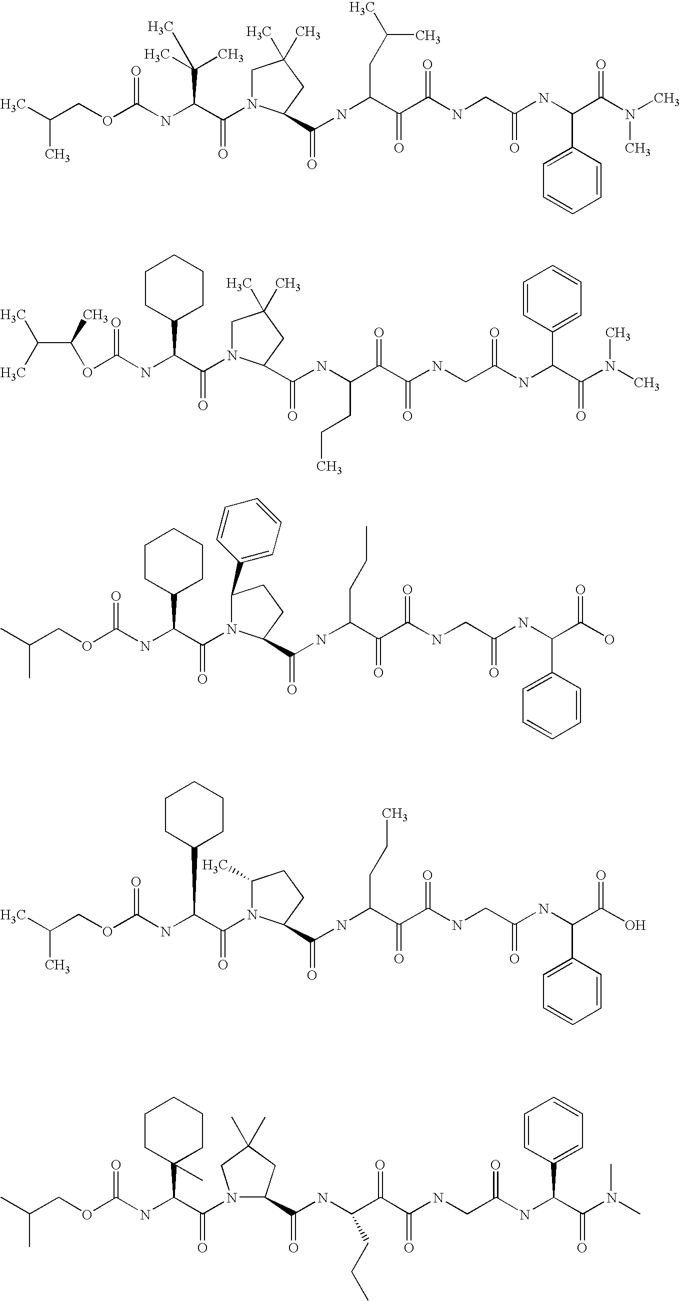 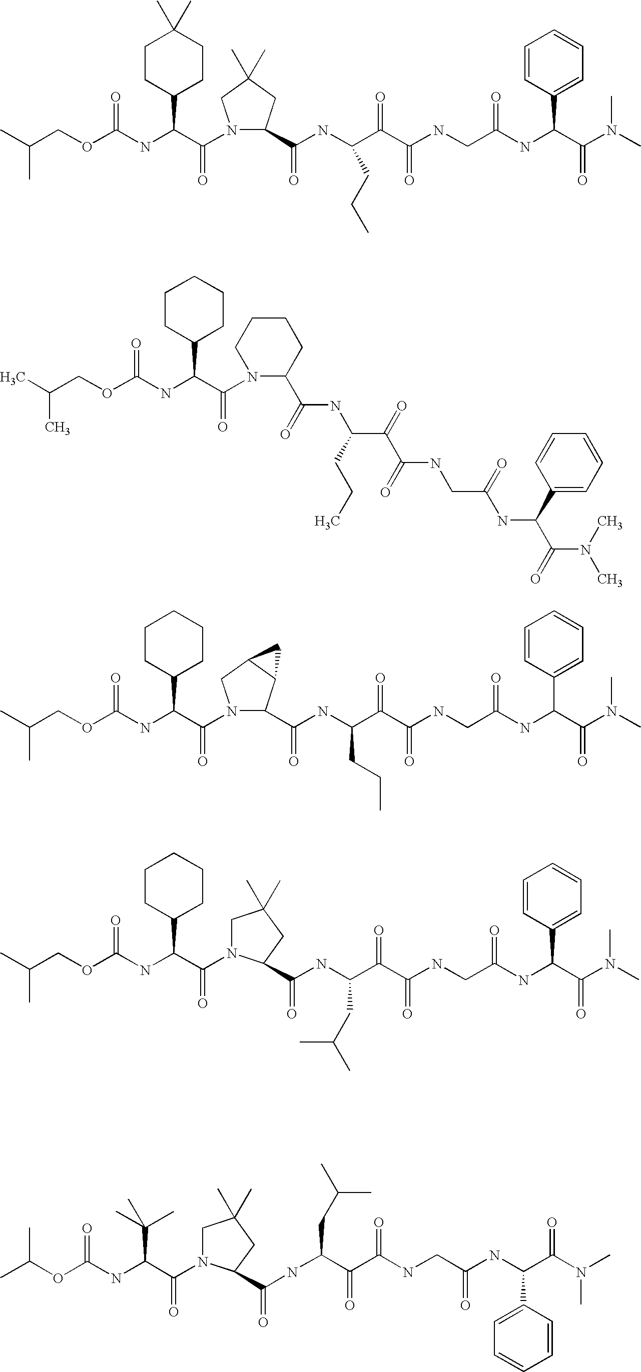 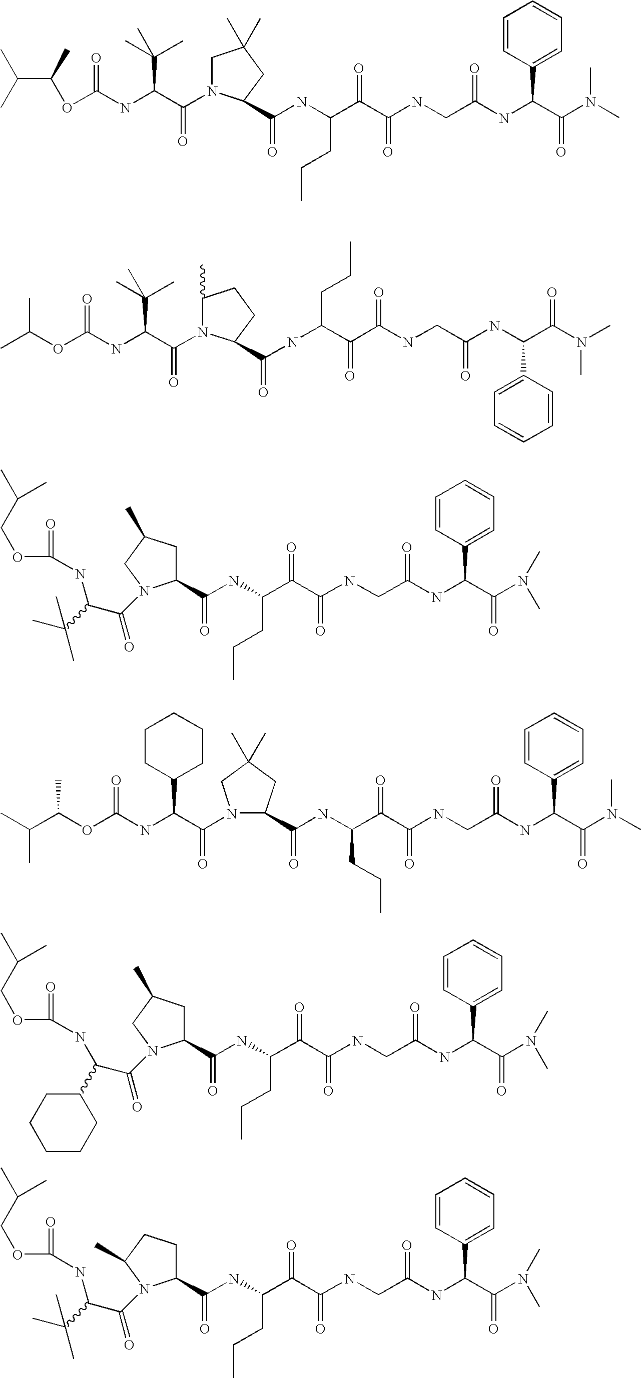 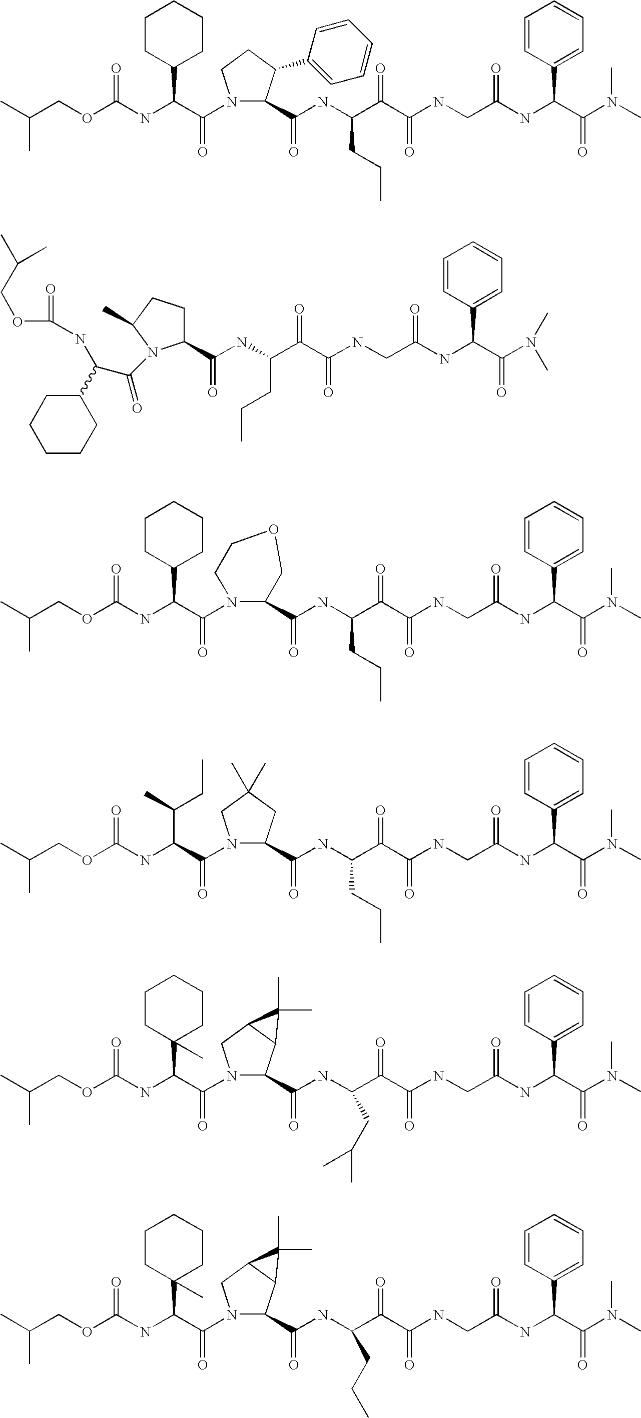 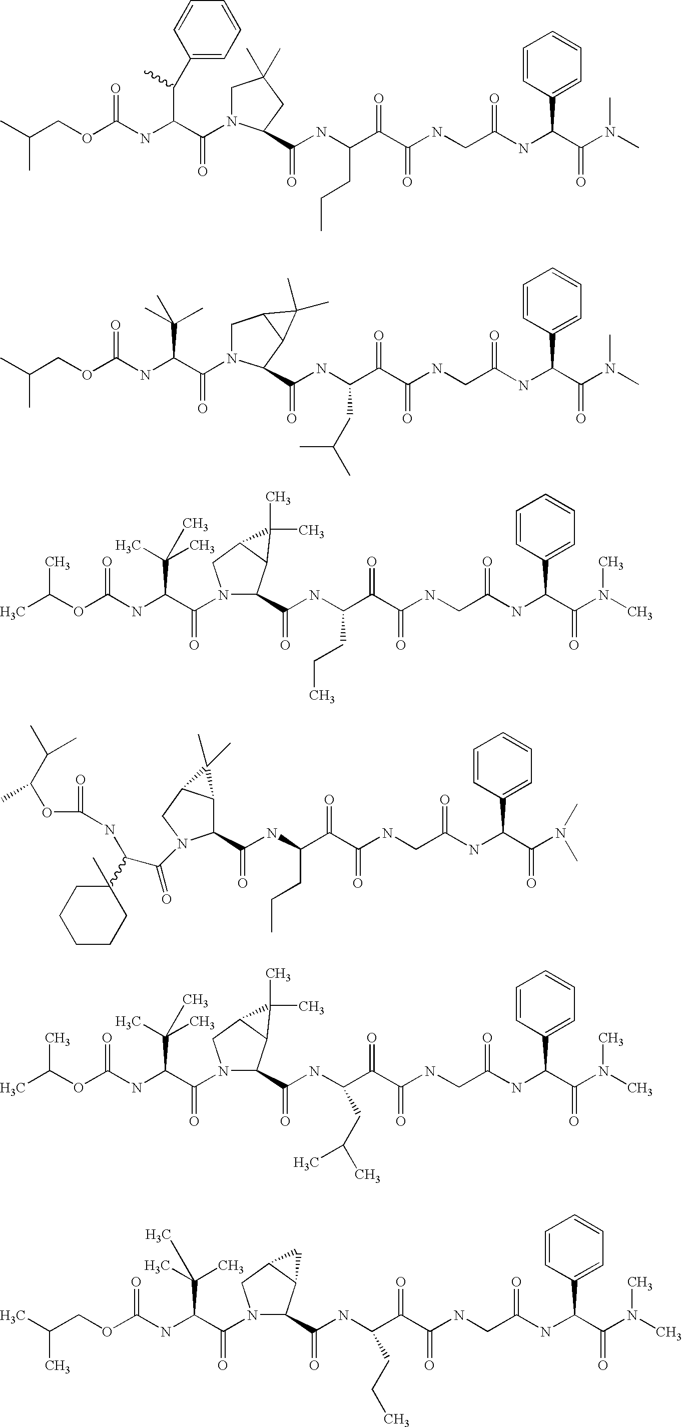 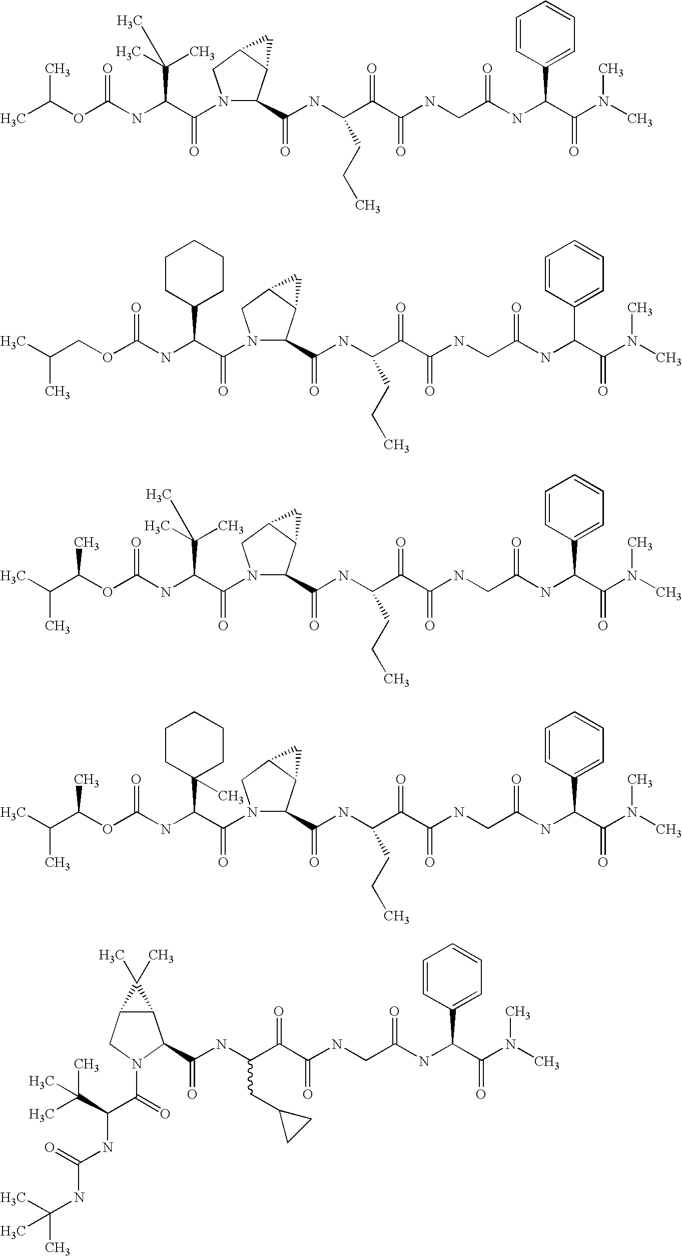 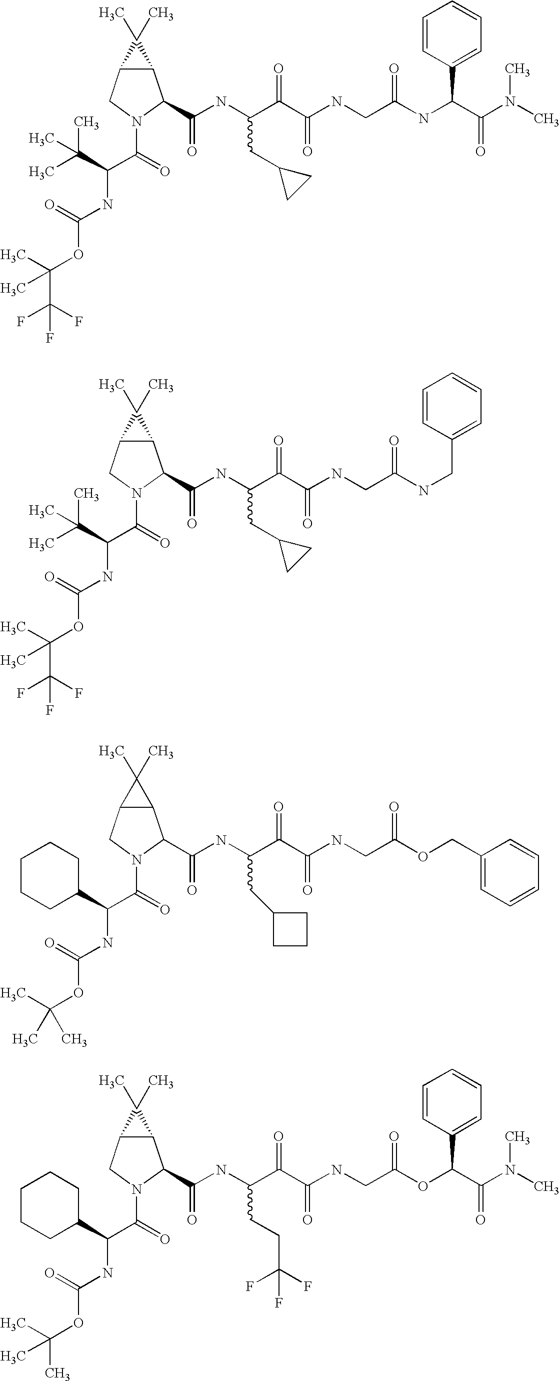 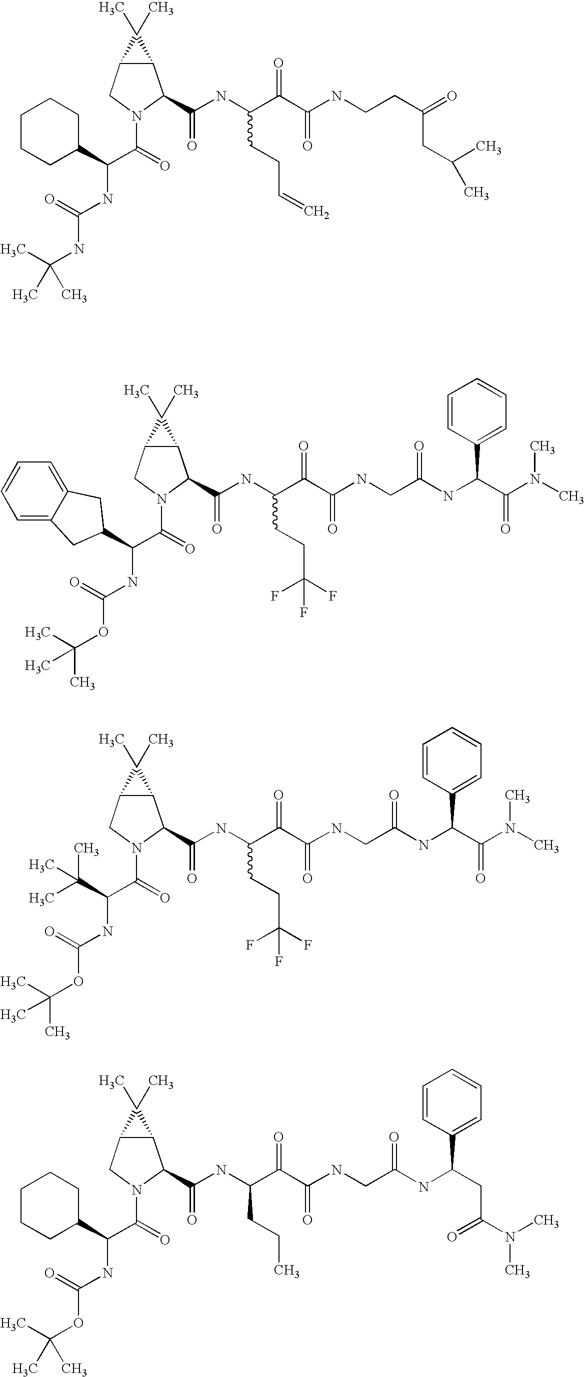 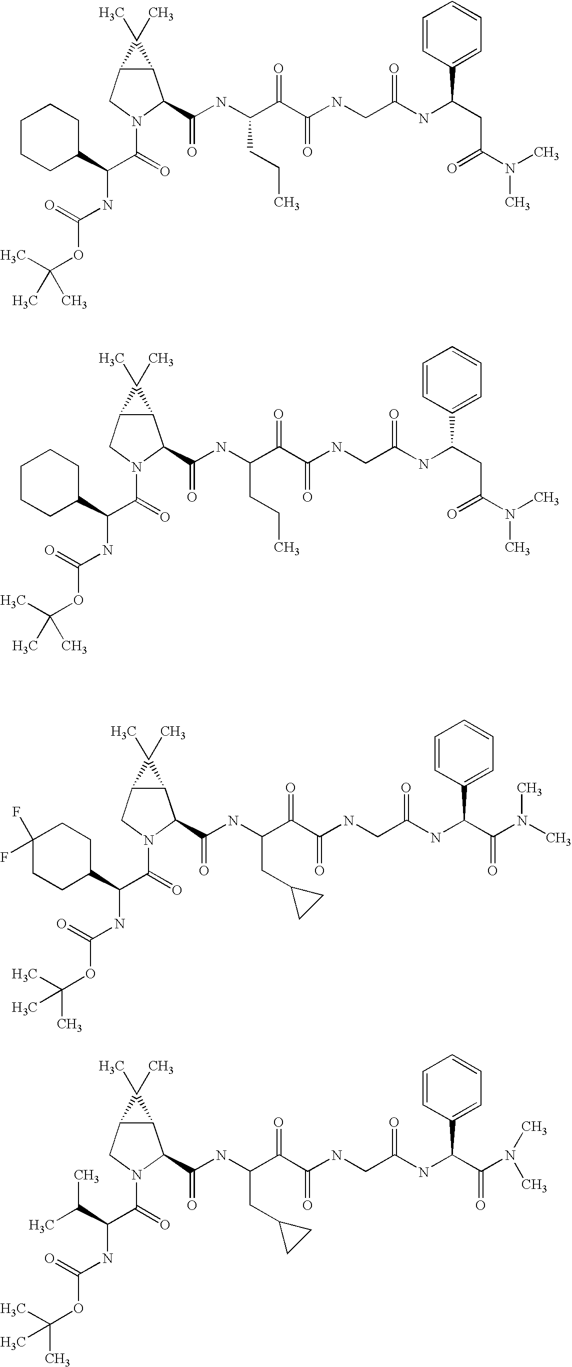 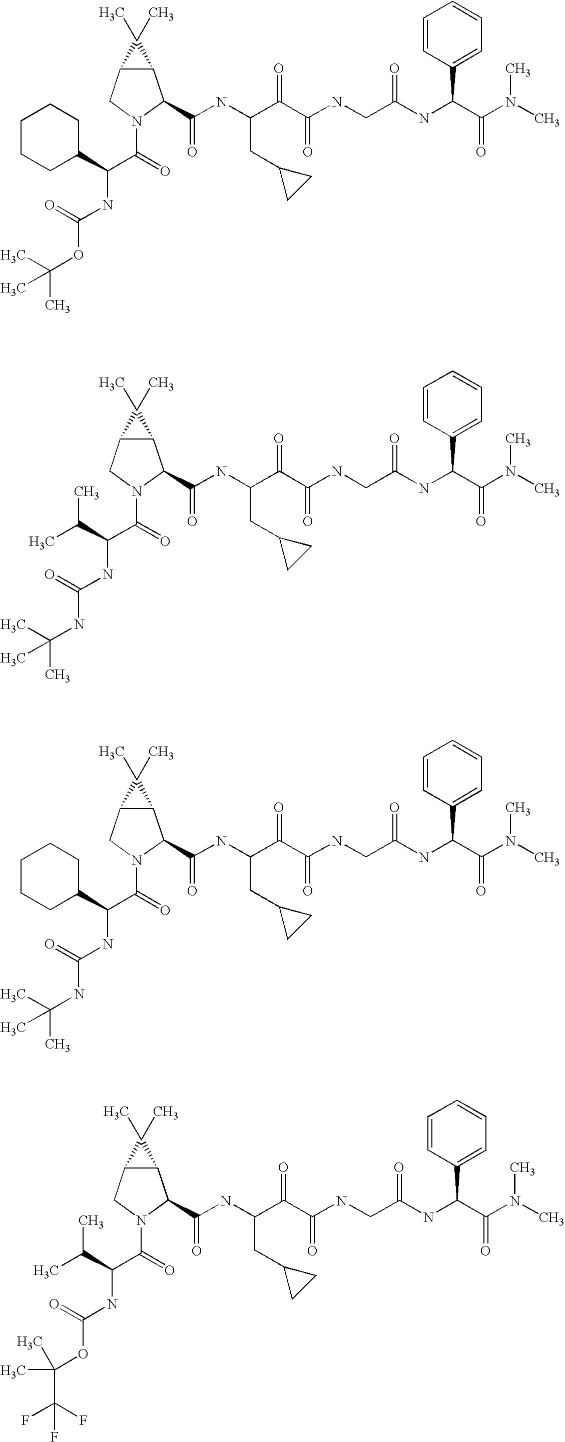 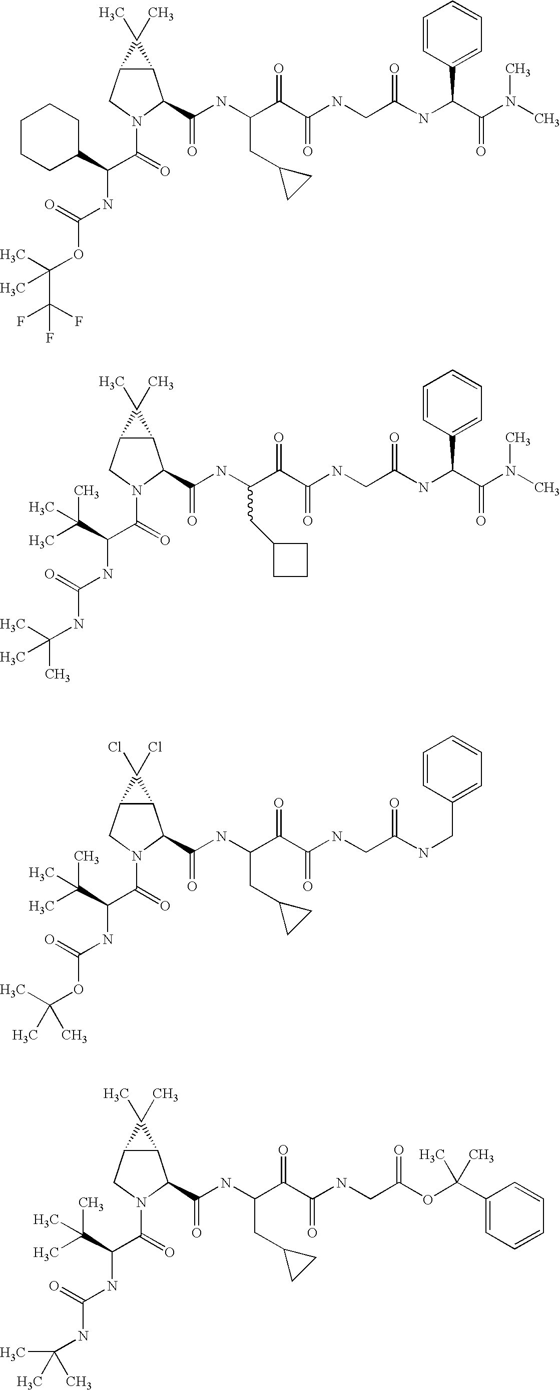 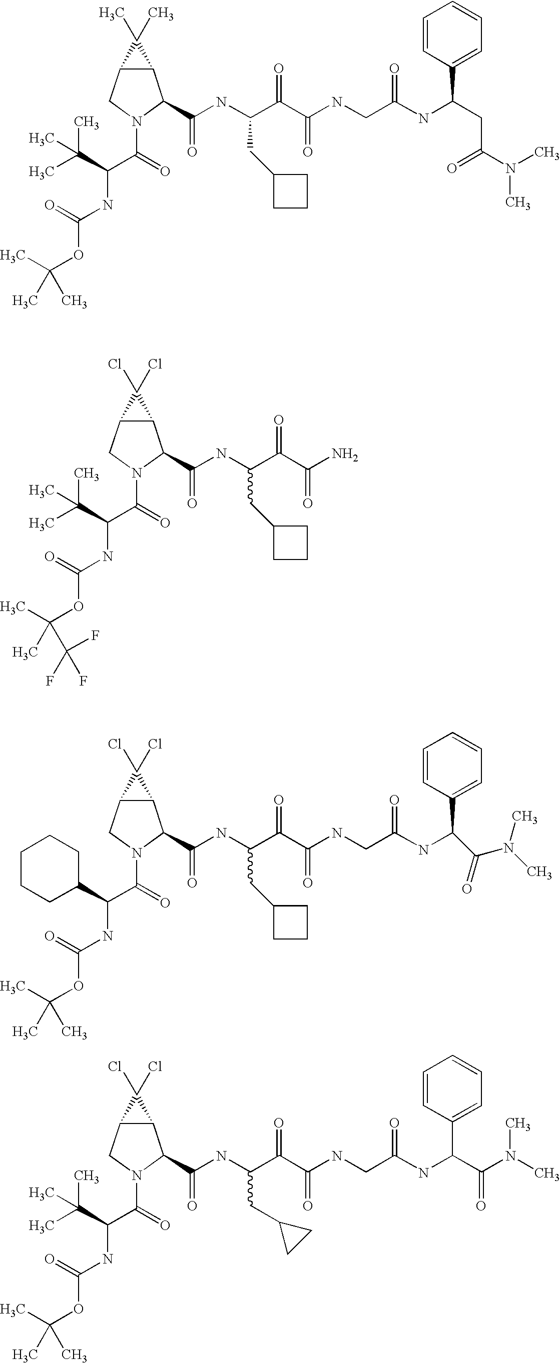 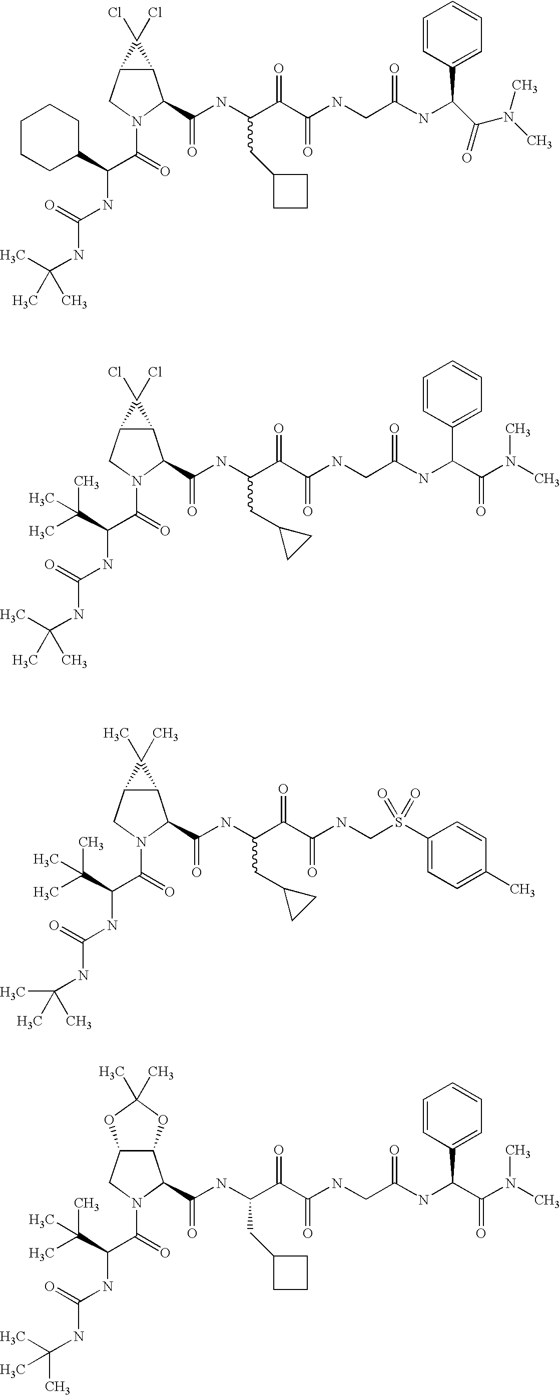 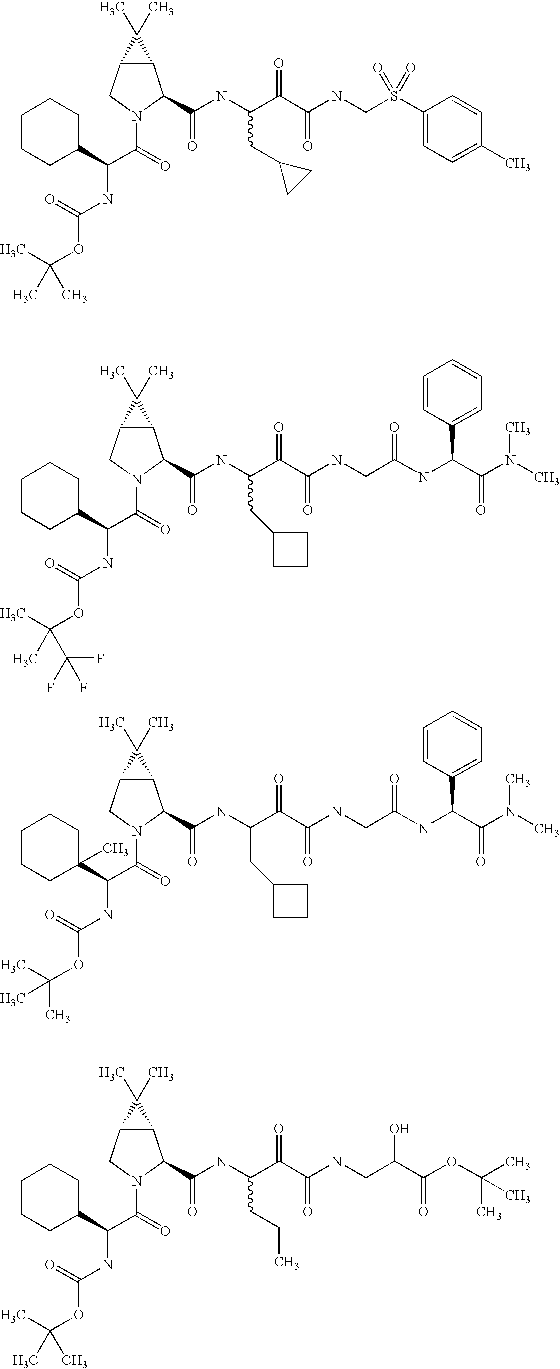 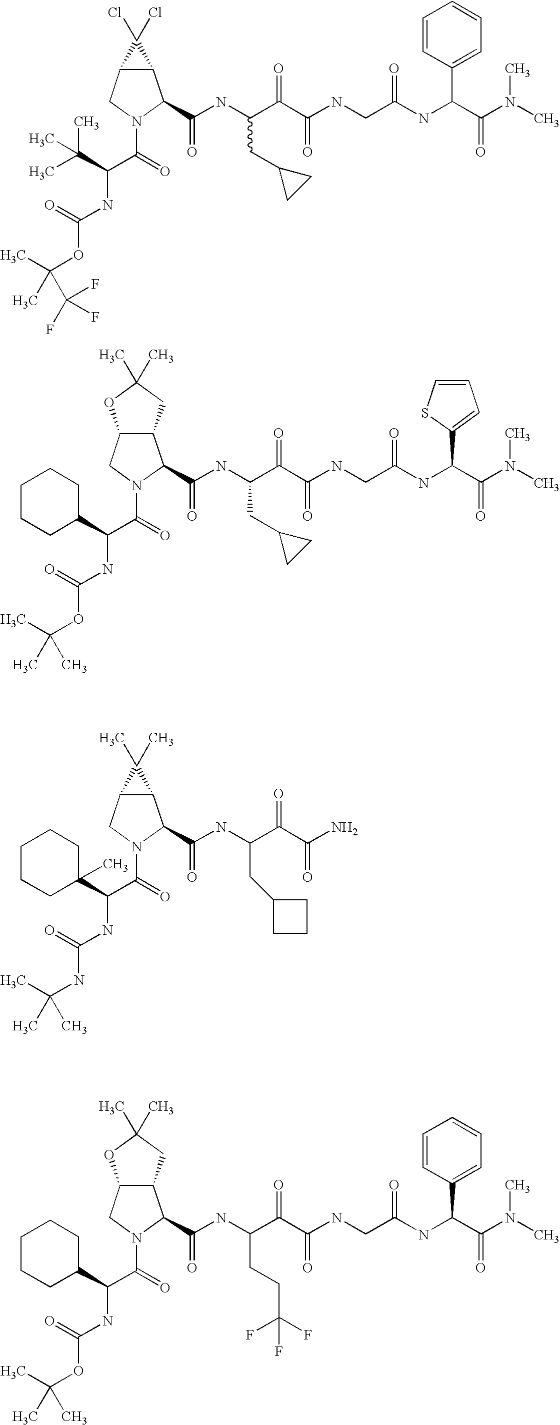 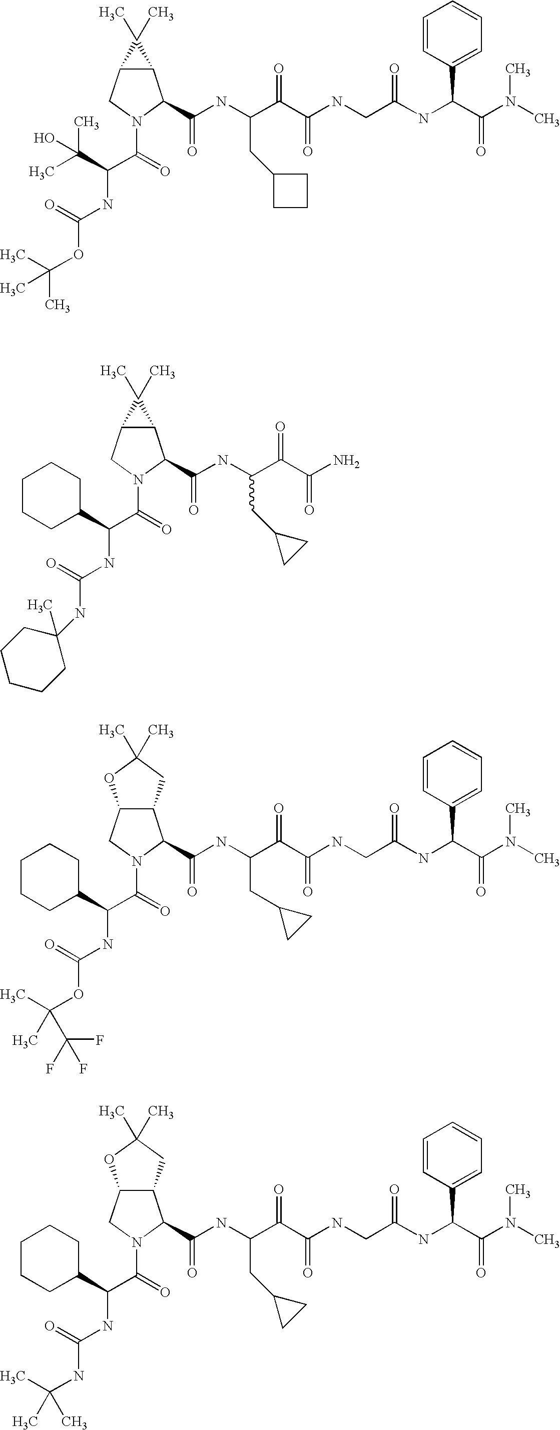 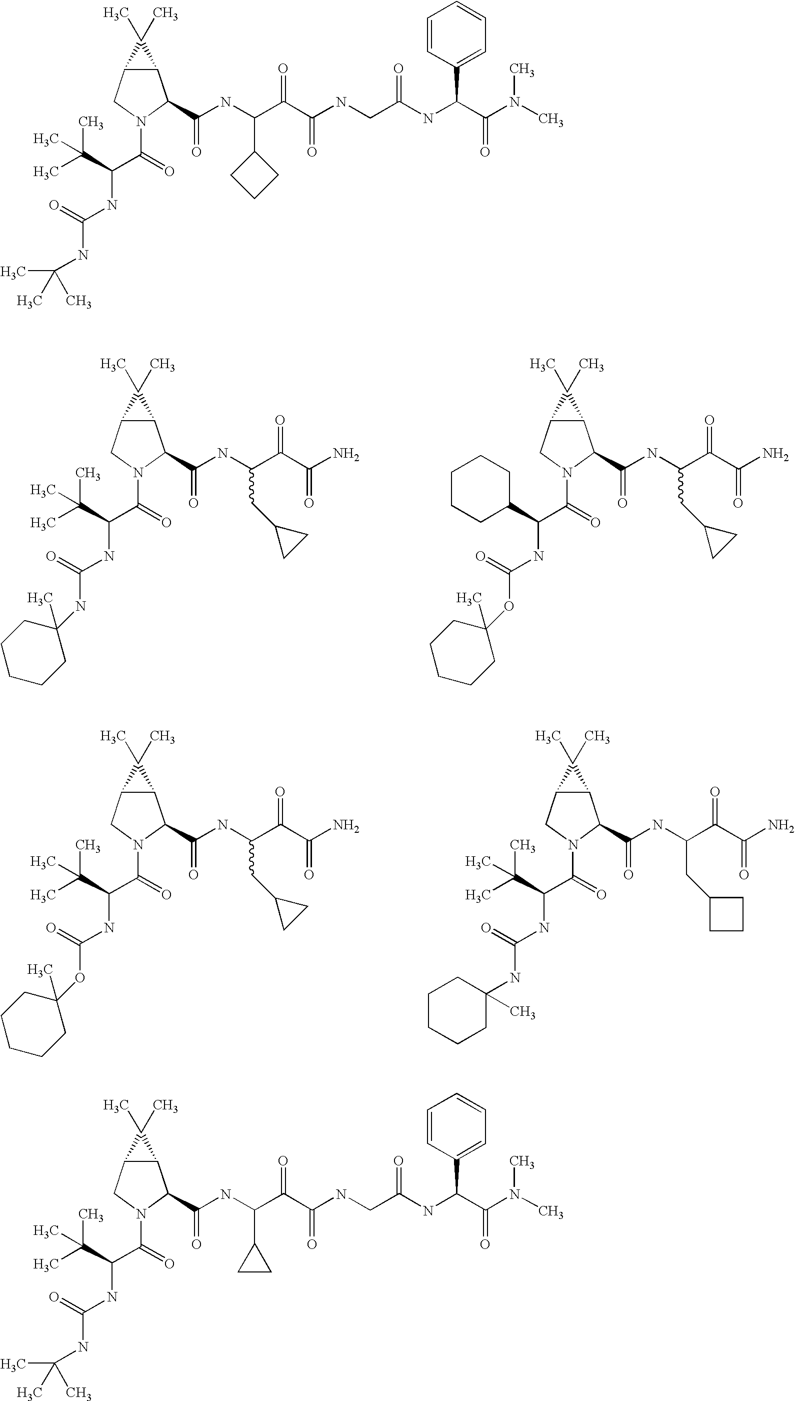 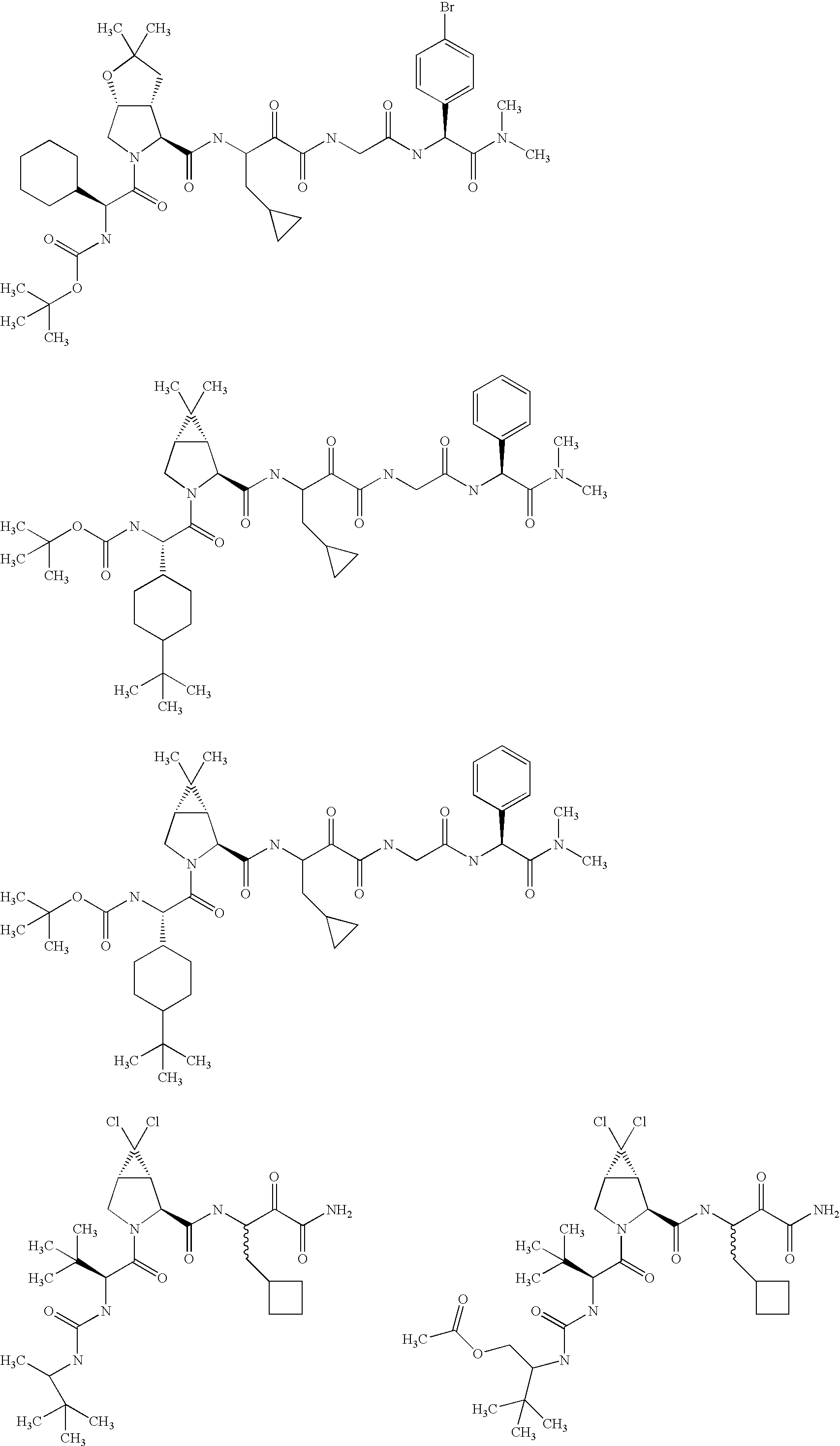 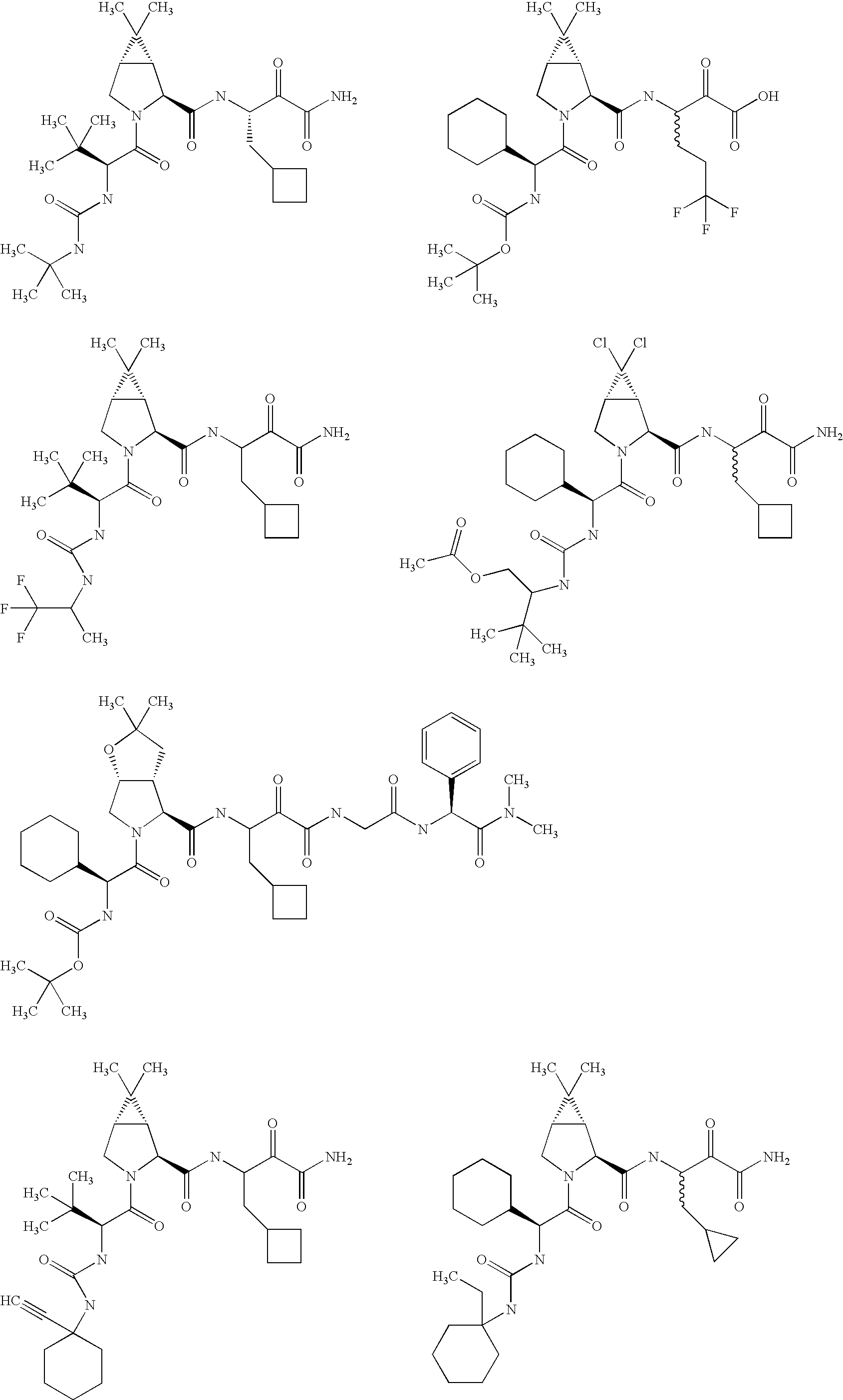 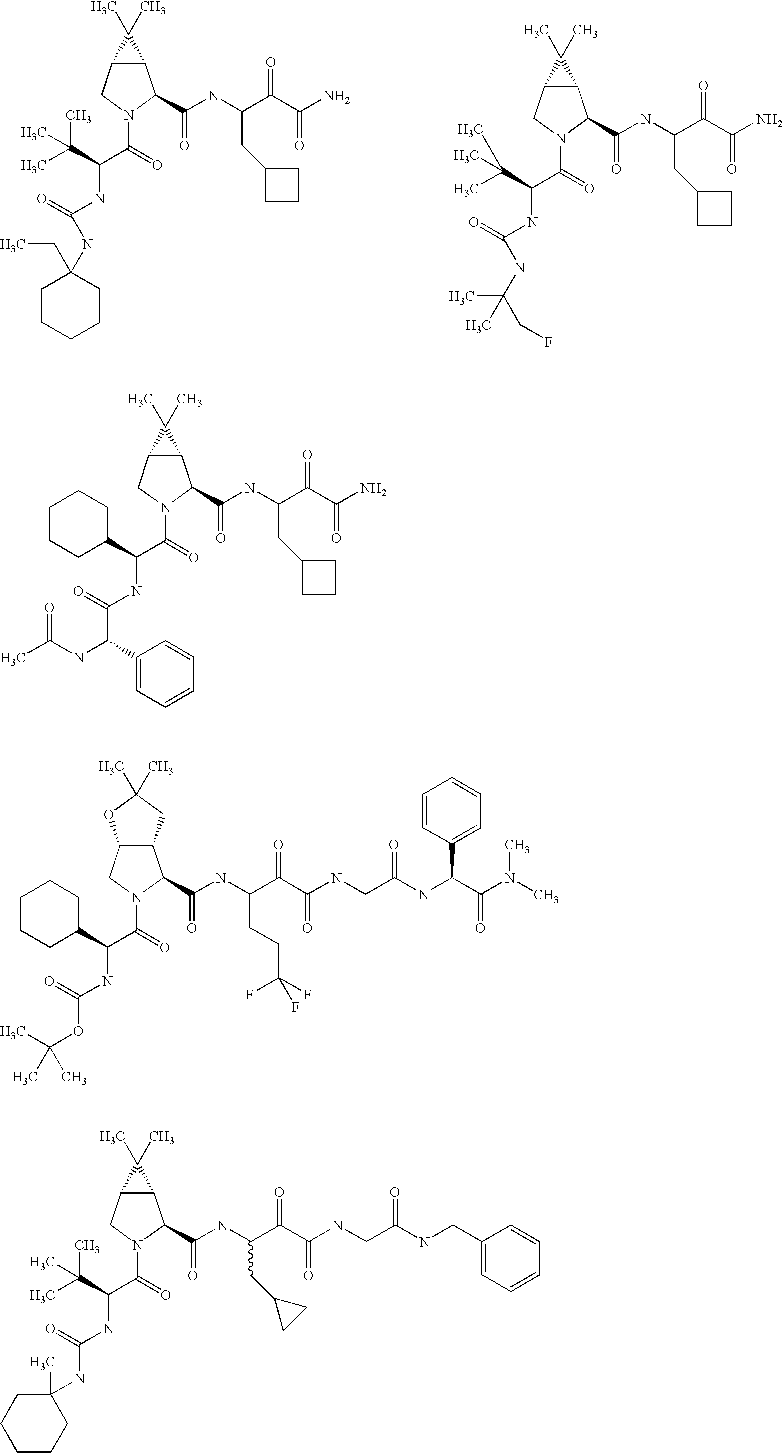 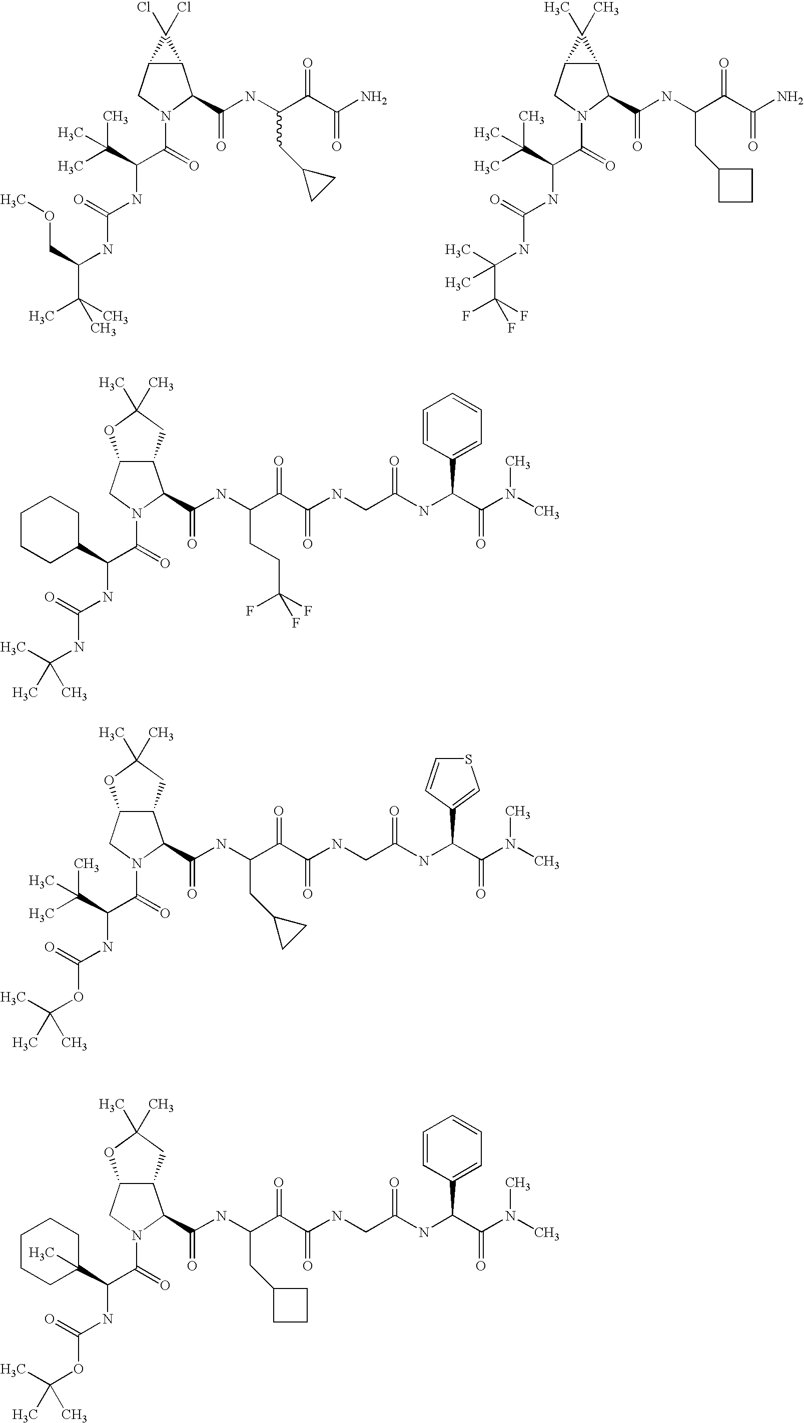 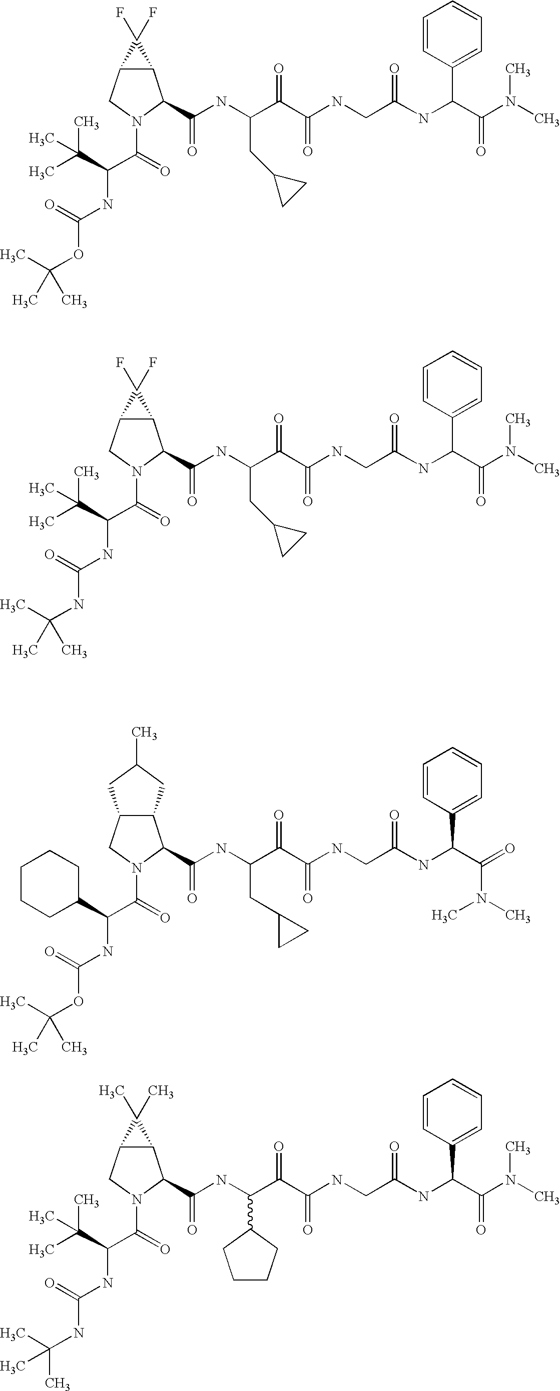 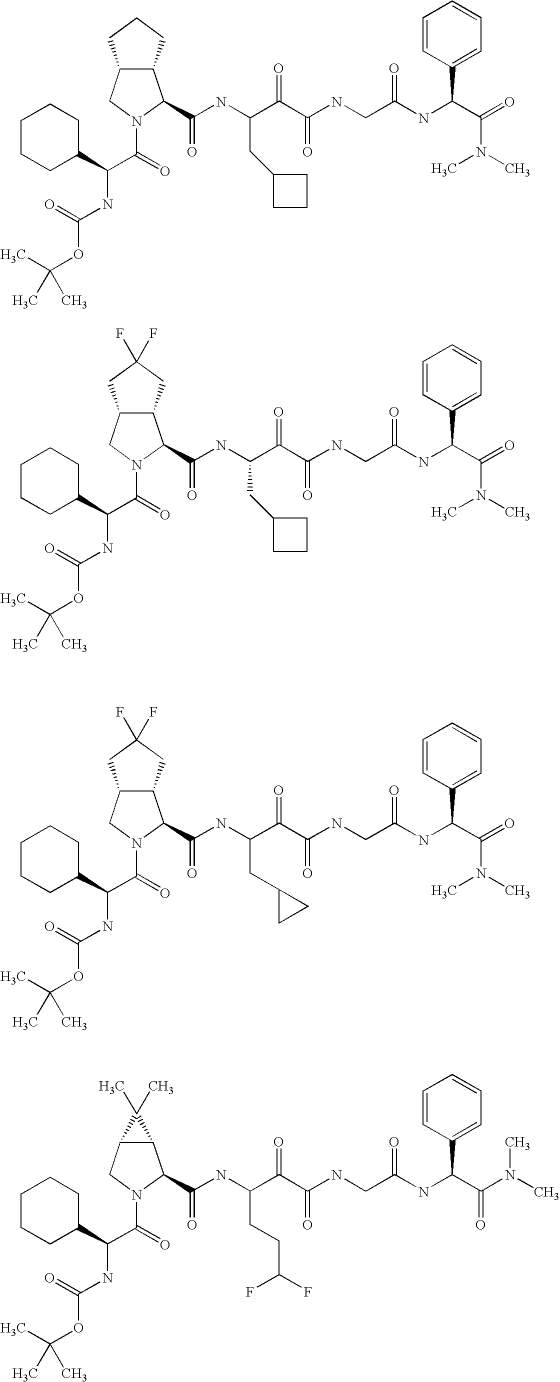 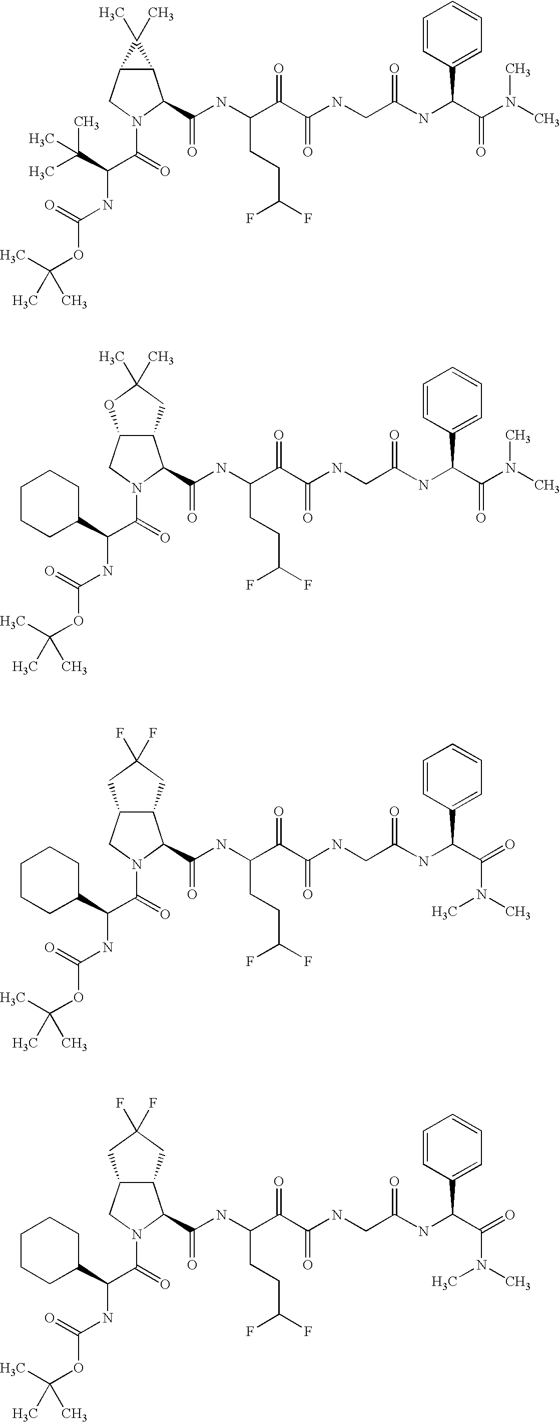 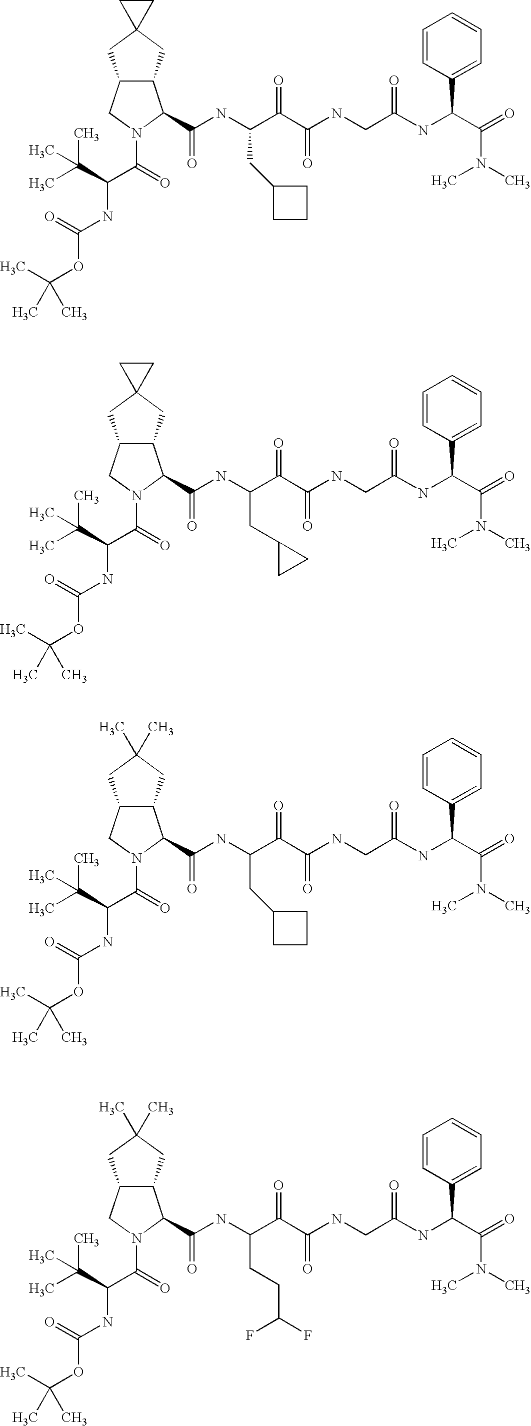 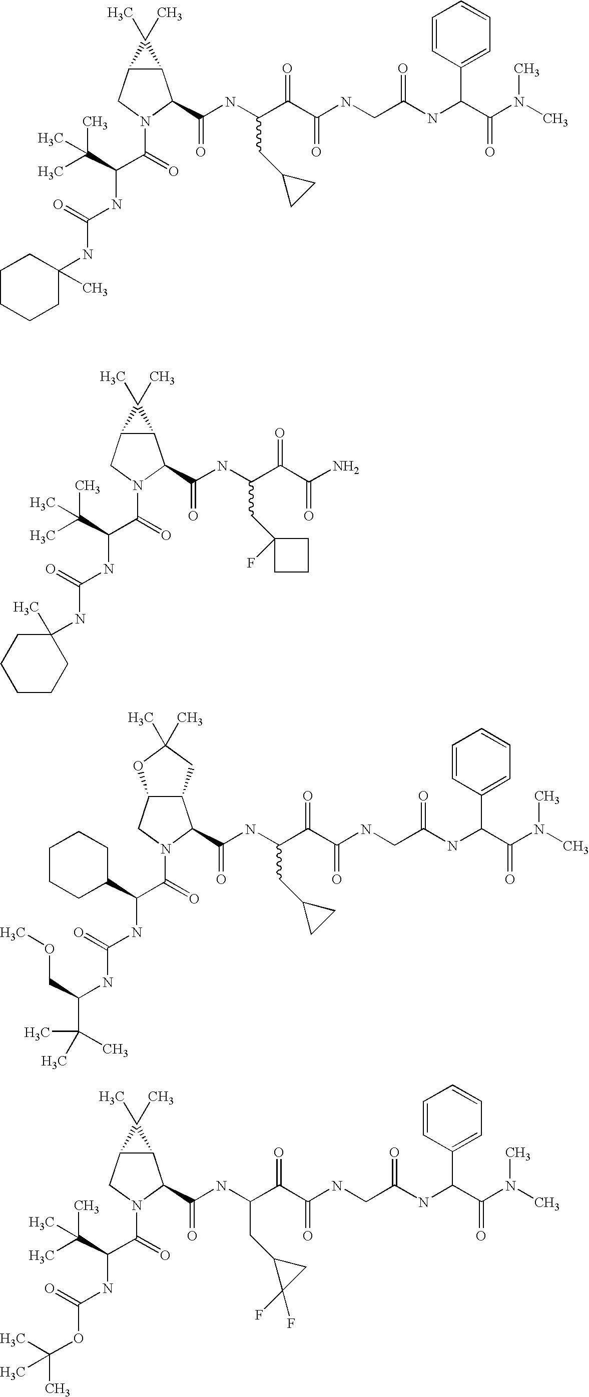 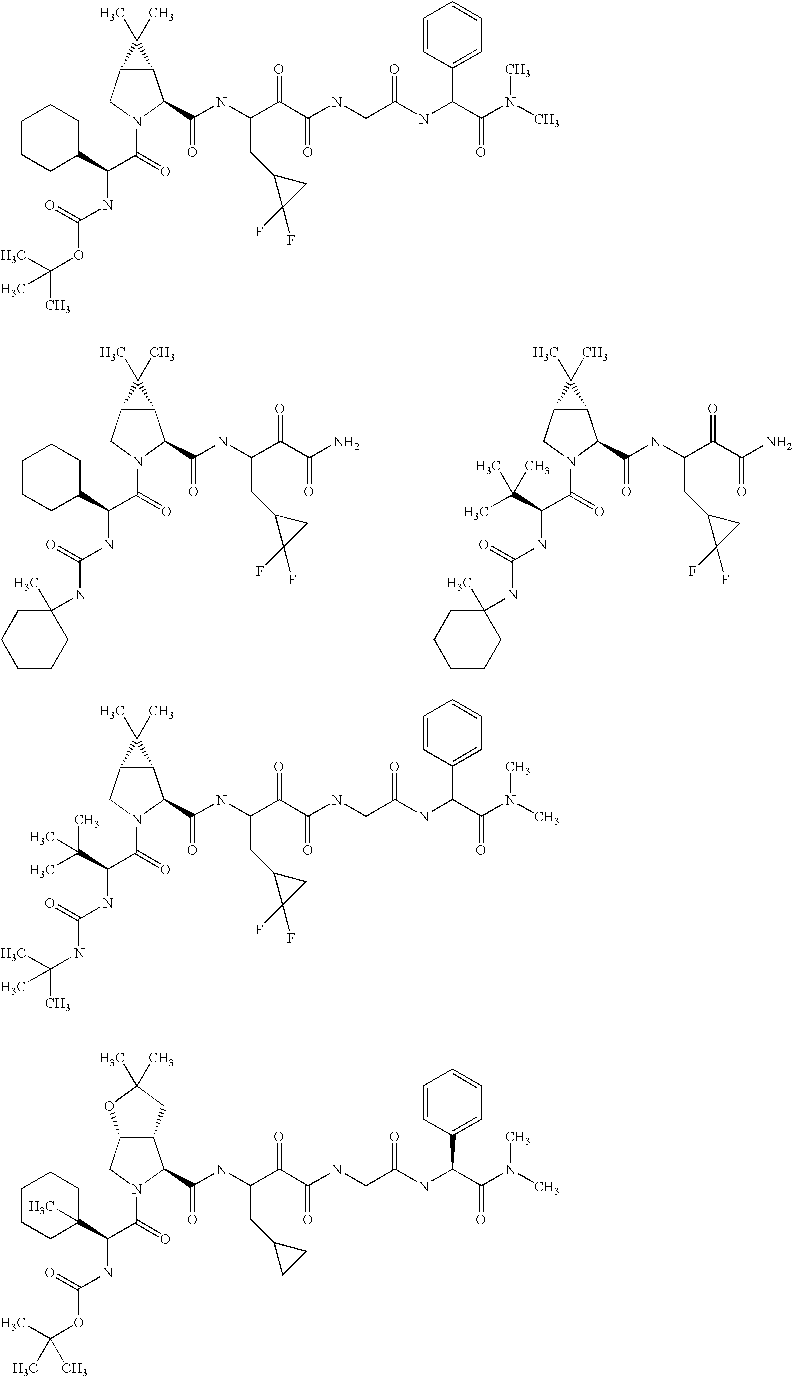 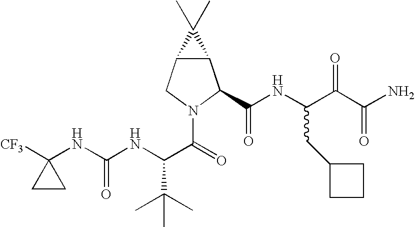
or a pharmaceutically acceptable salt, solvate or ester thereof.

3. A method of inhibiting cathepsin activity in a subject in need thereof comprising administering to said subject an effective amount of at least one compound of formula V: 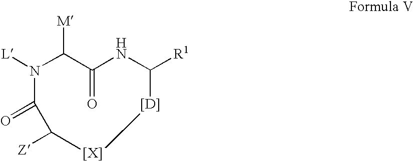
or a pharmaceutically acceptable salt, solvate or ester of said compound wherein:
(1) R¹ is —C(O)R⁵ or —B(OR)₂;
(2) R⁵ is H, —OH, —OR⁸, —NR⁹R¹⁰, —C(O)OR⁸, —C(O)NR⁹R¹⁰, —CF₃, —C₂F₅, C₃F₇, —CF₂R⁶, —R⁶, —C(O)R⁷ or NR⁷SO₂R⁸;
(3) R⁷ is H, —OH, —OR⁸,or —CHR⁹R¹⁰;
(4) R⁶, R⁸, R⁹ and R¹⁰ are independently selected from the group consisting of H: alkyl, alkenyl, aryl, heteroalkyl, heteroaryl, cycloalkyl, arylalkyl, heteroarylalkyl, R¹⁴, —CH(R^{1′})CH(R^{1′})C(O)OR¹¹,[CH(R^{1′})]ₚC(O)OR¹¹,—[CH(R^{1′})]ₚC(O)NR¹²R¹³,—[CH(R^{1′})]ₚS(O₂)R¹¹, —[CH(R^{1′})]ₚC(O)R¹¹, —[CH(R^{1′})]ₚS(O₂)NR¹²R¹³, CH(R^{1′})C(O)N(H)CH(R^{2′})(R′), CH(R^{1′})CH(R^{1′})C(O)NR¹²R¹³, —CH(R^{1′})CH(R^{1′})S(O₂)R¹¹, CH(R^{1′})CH(R^{1′})S(O₂)NR¹²R¹³, —CH(R^{1′})CH(R^{1′})C(O)R¹¹,—[CH(R^{1′})]ₚCH(OH)R¹¹,—CH(R^{1′})C(O)N(H)CH(R^{2′})C(O)OR¹¹, C(O)N(H)CH(R^{2′})C(O)OR¹¹,—C(O)N(H)CH(R^{2′})C(O)R¹¹,CH(R^{1′})C(O)N(H)CH(R^{2′})C(O) NR¹²R¹³,—CH(R^{1′})C(O)N(H)CH(R^{2′})R′,CH(R^{1′})C(O)N(H)CH(R^{2′})C(O)N(H)CH(R^{3′}) C(O)OR¹¹,CH(R^{1′})C(O)N(H)CH(R^{2′})C(O)CH(R^{3′})NR¹²R¹³,CH(R^{1′})C(O)N(H)CH(R^{2′})C(O) N(H)CH(R^{3′})C(O)NR¹²R¹³, CH(R^{1′})C(O)N(H)CH(R^{2′})C(O)N(H)CH(R^{3′})C(O)N(H)CH(R^{4′})C (O)OR¹¹,CH(R^{1′})C(O)N(H)CH(R^{2′})C(O)N(H)CH(R^{3′})C(O)N(H)CH(R^{4′})C(O)NR¹²R¹³, CH(R^{1′})C(O)N(H)CH(R²)C(O)N(H)CH(R^{3′})C(O)N(H)CH(R^{4′})C(O)N(H)CH(R^{5′})C(O)OR¹¹, andCH(R^{1′})C(O)N(H)CH(R^{2′})C(O)N(H)CH(R^{3′})C(O)N(H)CH(R^{4′})C(O)N(H)CH(R^{5′}) C(O)NR¹²R¹³;
wherein R^{1′}, R^{2′}, R^{3′}, R^{4′}, R^{5′}, R¹¹, R¹²and R¹³ can be the same or different, each being independently selected from the group consisting of: H, halogen, alkyl, aryl, heteroalkyl, heteroaryl, cycloalkyl, alkoxy, aryloxy, alkenyl, alkynyl, alkyl-aryl, alkyl-heteroaryl, heterocycloalkyl, aryl-alkyl and heteroaralkyl; or
R¹² and R¹³ are linked together wherein the combination is cycloalkyl, heterocycloalkyl, ary or heteroaryl;
R¹⁴ is present or not and if present is selected from the group consisting of: H, alkyl, aryl, heteroalkyl, heteroaryl, cycloalkyl, alkyl-aryl, allyl, alkyl-heteroaryl, alkoxy, aryl-alkyl, alkenyl, alkynyl and heteroaralkyl;
(5) R and R′ are present or not and if present can be the same or different, each being independently selected from the group consisting of: H, OH, C₁-C₁₀ alkyl, C₂-C₁₀ alkenyl, C₃-C₈ cycloalkyl, C₃-C₈ heterocycloalkyl, alkoxy, aryloxy, alkylthio, arylthio, alkylamino, arylamino, amino, amido, arylthioamino, arylcarbonylamino, arylaminocarboxy, alkylaminocarboxy, heteroalkyl, alkenyl, alkynyl, (aryl)alkyl, heteroarylalkyl, ester, carboxylic acid, carbamate, urea, ketone, aldehyde, cyano, nitro, halogen, (cycloalkyl)alkyl, aryl, heteroaryl, (alkyl)aryl, alkylheteroaryl, alkyl-heteroaryl and (heterocycloalkyl)alkyl, wherein said cycloalkyl is made of three to eight carbon atoms, and zero to six oxygen, nitrogen, sulfur, or phosphorus atoms, and said alkyl is of one to six carbon atoms;
(6) L′ is H, OH, alkyl, heteroalkyl, aryl, heteroaryl, cycloalkyl, or heterocyclyl;
(7) M′ is H, alkyl, heteroalkyl, aryl, heteroaryl, cycloalkyl, arylalkyl, heterocyclyl or an amino acid side chain;
or L′ and M′ are linked together to form a ring structure wherein the portion of structural Formula 1 represented by 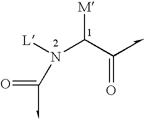
is represented by structural Formula 2: 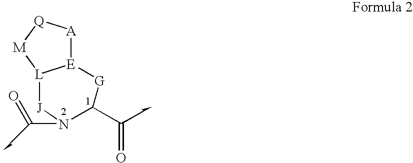
wherein in Formula 2:
E is present or absent and if present is C, CH, N or C(R);
J is present or absent, and when J is present, J is (CH₂)ₚ, (CHR—CHR′)ₚ, (CHR)ₚ, (CRR′)ₚ, S(O₂), N(H), N(R) or O; when J is absent and G is present, L is directly linked to the nitrogen atom marked position 2;
p is a number from 0 to 6;
L is present or absent, and when L is present, L is C(H) or C(R); when L is absent, M is present or absent; if M is present with L being absent, then M is directly and independently linked to E, and J is directly and independently linked to E;
G is present or absent, and when G is present, G is (CH₂)ₚ, (CHR)ₚ, (CHR—CHR′)ₚ or (CRR′)ₚ; when G is absent, J is present and E is directly connected to the carbon atom marked position 1;
Q is present or absent, and when Q is present, Q is NR, PR, (CR═CR), (CH₂)ₚ, (CHR)ₚ, (CRR′)ₚ, (CHR—CHR′)ₚ, O, NR, S, SO, or SO₂; when Q is absent, M is (i) either directly linked to A or (ii) an independent substituent on L, said independent substituent bing selected from —OR, —CH(R)(R′), S(O)₀₋₂R or —NRR′ or (iii) absent; when both Q and M are absent, A is either directly linked to L, or A is an independent substituent on E, said independent substituent bing selected from —OR, —CH(R)(R′), S(O)₀₋₂R or —NRR′ or A is absent;
A is present or absent and if present A is O, O(R), (CH₂)ₚ, (CHR)ₚ, (CHR—CHR′)ₚ, (CRR′)ₚ, N(R), NRR′, S, S(O₂), —OR, CH(R)(R′) or NRR′; or A is linked to M to form an alicyclic, aliphatic or heteroalicyclic bridge;
M is present or absent, and when M is present, M is halogen, O, OR, N(R), S, S(O₂), (CH₂)ₚ, (CHR)ₚ (CHR—CHR′)ₚ, or (CRR′)ₚ; or M is linked to A to form an alicyclic, aliphatic or heteroalicyclic bridge;
(8) Z′ is represented by the structural Formula 3: 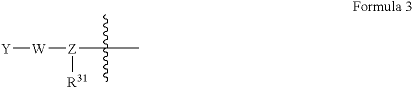
wherein in Formula 3, Y is selected from the group consisting of: H, aryl, alkyl, alkyl-aryl, heteroalkyl, heteroaryl, aryl-heteroaryl, alkyl-heteroaryl, cycloalkyl, alkyloxy, alkyl-aryloxy, aryloxy, heteroaryloxy, heterocycloalkyloxy, heteroalkyl-heteroaryl, heteroalkyl-heterocycloalkyl, cycloalkyloxy, alkylamino, arylamino, alkyl-arylamino, arylamino, heteroarylamino, cycloalkylamino and heterocycloalkylamino, and Y is unsubstituted or optionally substituted with one or two substituents which are the same or different and are independently selected from X¹¹ or X¹²;
X¹¹ is alkyl, alkenyl, alkynyl, cycloalkyl, cycloalkyl-alkyl, heterocyclyl, heterocyclylalkyl, aryl, alkylaryl, arylalkyl, heteroaryl, alkylheteroaryl, or heteroarylalkyl, and X¹¹ is unsubstituted or optionally substituted with one or more of X¹² moieties which are the same or different and are independently selected;
X¹² is hydroxy, alkoxy, alkyl, alkenyl, alkynyl, aryl, aryloxy, thio, alkylthio, arylthio, amino, alkylamino, arylamino, alkylsulfonyl, arylsulfonyl, alkylsulfonamido, arylsulfonamido, carboxy, carbalkoxy, carboxamido, alkylcarbonyl, arylcarbonyl, heteroalkylcarbonyl, heteroarylcarbonyl,sulfonylurea,cycloalkylsulfonamido, heteroaryl-cycloalkylsulfonamido, heteroaryl-sulfonamido, alkoxycarbonylamino, alkoxycarbonyloxy, alkylureido, arylureido, halogen, cyano, or nitro, and said alkyl, alkoxy, and aryl are unsubstituted or optionally independently substituted with one or more moieties which are the same or different and are independently selected from alkyl, alkenyl, alkynyl, cycloalkyl, cycloalkyl-alkyl, heterocyclyl, heterocyclylalkyl, aryl, alkylaryl, arylalkyl, heteroaryl, alkylheteroaryl, or heteroarylalkyl;
Z is O, N, C(H) or C(R);
R³¹ is H, hydroxyl, aryl, alkyl, alkyl-aryl, heteroalkyl, heteroaryl, aryl-heteroaryl, alkyl-heteroaryl, cycloalkyl, alkyloxy, alkyl-aryloxy, aryloxy, heteroaryloxy, heterocycloalkyloxy, heteroalkyl-heteroaryl, cycloalkyloxy, alkylamino, arylamino, alkyl-arylamino, arylamino, heteroarylamino, cycloalkylamino or heterocycloalkylamino, and R³¹ is unsubstituted or optionally substituted with one or two substituents which are the same or different and are independently selected from X¹³ or X¹⁴;
X¹³ is alkyl, alkenyl, alkynyl, cycloalkyl, cycloalkyl-alkyl, heterocyclyl, heterocyclylalkyl, aryl, alkylaryl, arylalkyl, heteroaryl, alkylheteroaryl, or heteroarylalkyl, and X¹³ is unsubstituted or optionally substituted with one or more of X¹⁴ moieties which are the same or different and are independently selected;
X¹⁴ is hydroxy, alkoxy, alkyl, alkenyl, alkynyl, aryl, aryloxy, thio, alkylthio, arylthio, amino, alkylamino, arylamino, alkylsulfonyl, arylsulfonyl, alkylsulfonamido, arylsulfonamido, carboxy, carbalkoxy, carboxamido, alkylcarbonyl, arylcarbonyl, heteroalkylcarbonyl, heteroarylcarbonyl, cycloalkylsulfonamido, heteroaryl-cycloalkylsulfonamido, heteroarylsulfonamido, alkoxycarbonylamino, alkoxycarbonyloxy, alkylureido, arylureido, halogen, cyano, or nitro, and said alkyl, alkoxy, and aryl are unsubstiuted or optionally independently substituted with one or more moieties which are the same or different and are independently selected from alkyl, alkenyl, alkynyl, cycloalkyl, cycloalkyl-alkyl, heterocyclyl, heterocyclylalkyl, aryl, alkylaryl, arylalkyl, heteroaryl, alkylheteroaryl, or heteroarylalkyl;
W may be present or absent, and if W is present, W is C(═O), C(═S), C(═N—CN), or S(O₂);
(9) X is represented by structural Formula 4: 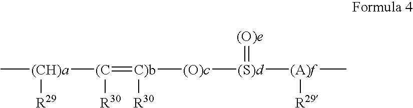
wherein in Formula 4, a is 2, 3, 4, 5, 6, 7, 8 or 9; b, c, d, e and f are 0, 1, 2, 3, 4 or 5;
A is C, N, S or O;
R²⁹ and R^{29′} are independently present or absent and if present can be the same or different, each being independently one or two substituents independently selected from the group consisting of: H, halo, alkyl, aryl, cycloalkyl, cycloalkylamino, cycloalkylaminocarbonyl, cyano, hydroxy, alkoxy, alkylthio, amino, —NH(alkyl), —NH(cycloalkyl), —N(alkyl)₂, carboxyl, C(O)O-alkyl, heteroaryl, aralkyl, alkylaryl, aralkenyl, heteroaralkyl, alkylheteroaryl, heteroaralkenyl, hydroxyalkyl, aryloxy, aralkoxy, acyl, aroyl, nitro, aryloxycarbonyl, aralkoxycarbonyl, alkylsulfonyl, arylsulfonyl, heteroarylsulfonyl, alkylsulfinyl, arylsulfinyl, heteroarylsulfinyl, arylthio, heteroarylthio, aralkylthio, heteroaralkylthio, cycloalkenyl, heterocyclyl, heterocyclenyl, Y₁Y₂N-alkyl-, Y₁Y₂NC(O)— and Y₁Y₂NSO₂—, wherein Y₁ and Y₂ can be the same or different and are independently selected from the group consisting of hydrogen, alkyl, aryl, and aralkyl; or
R²⁹ and R^{29′} are linked together such that the combination is an aliphatic or heteroaliphatic chain of 0 to 6 carbons;
R³⁰ is present or absent and if present is one or two substituents independently selected from the group consisting of: H, alkyl, aryl, heteroaryl and cylcoalkyl;
(10) D is represented by structural Formula 5: 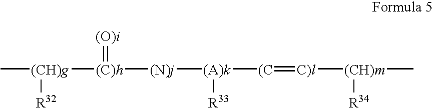
wherein in Formula 5, R³², R³³ and R³⁴ are present or absent and if present are independently one or two substituents independently selected from the group consisting of: H, halo, alkyl, aryl, cycloalkyl, cycloalkylamino, spiroalkyl, cycloalkylaminocarbonyl, cyano, hydroxy, alkoxy, alkylthio, amino, —NH(alkyl), —NH(cycloalkyl), —N(alkyl)₂, carboxyl, —C(O)O-alkyl, heteroaryl, aralkyl, alkylaryl, aralkenyl, heteroaralkyl, alkylheteroaryl, heteroaralkenyl, hydroxyalkyl, aryloxy, aralkoxy, acyl, aroyl, nitro, aryloxycarbonyl, aralkoxycarbonyl, alkylsulfonyl, arylsulfonyl, heteroarylsulfonyl, alkylsulfinyl, arylsulfinyl, heteroarylsulfinyl, arylthio, heteroarylthio, aralkylthio, heteroaralkylthio, cycloalkenyl, heterocyclyl, heterocyclenyl, Y₁Y₂N-alkyl-, Y₁Y₂NC(O)— and Y₁Y₂NSO₂—, wherein Y₁ and Y₂ can be the same or different and are independently selected from the group consisting of hydrogen, alkyl, aryl, and aralkyl; or
R³² and R³⁴ are linked together such that the combination forms a portion of a cycloalkyl group;
g is 1, 2, 3, 4, 5, 6, 7, 8 or 9;
h, i, j, k, I and m are 0, 1, 2, 3, 4 or 5; and
A is C, N, S or O,
(11) provided that when structural Formula 2: 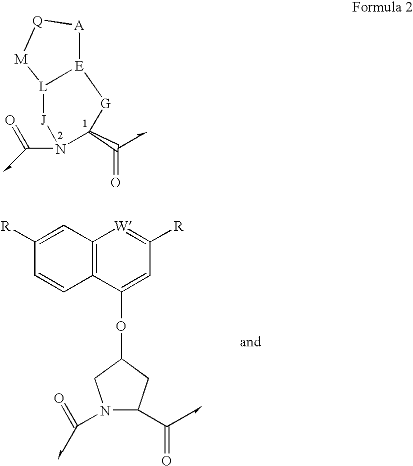
W′ is CH or N, both the following conditional exclusions (i) and (ii) apply: conditional exclusion (i): Z′ is not —NH—R³⁶, wherein R³⁶ is H, C_{6 or 10} aryl, heteroaryl, —C(O)—R³⁷, —C(O)—OR³⁷ or —C(O)—NHR³⁷, wherein R³⁷ is C₁₋₆ alkyl or C₃₋₆ cycloalkyl; and
conditional exclusion (ii): R¹ is not —C(O)OH, a pharmaceutically acceptable salt of —C(O)OH, an ester of —C(O)OH or —C(O)NHR³⁸ wherein R³⁸ is selected from the group consisting of C₁₋₈ alkyl, C₃₋₆ cycloalkyl, C_{6 to 10} aryl or C₇₋₁₆ aralkyl.

4. The method of claim 3, wherein said compound is selected from the group consisting of: 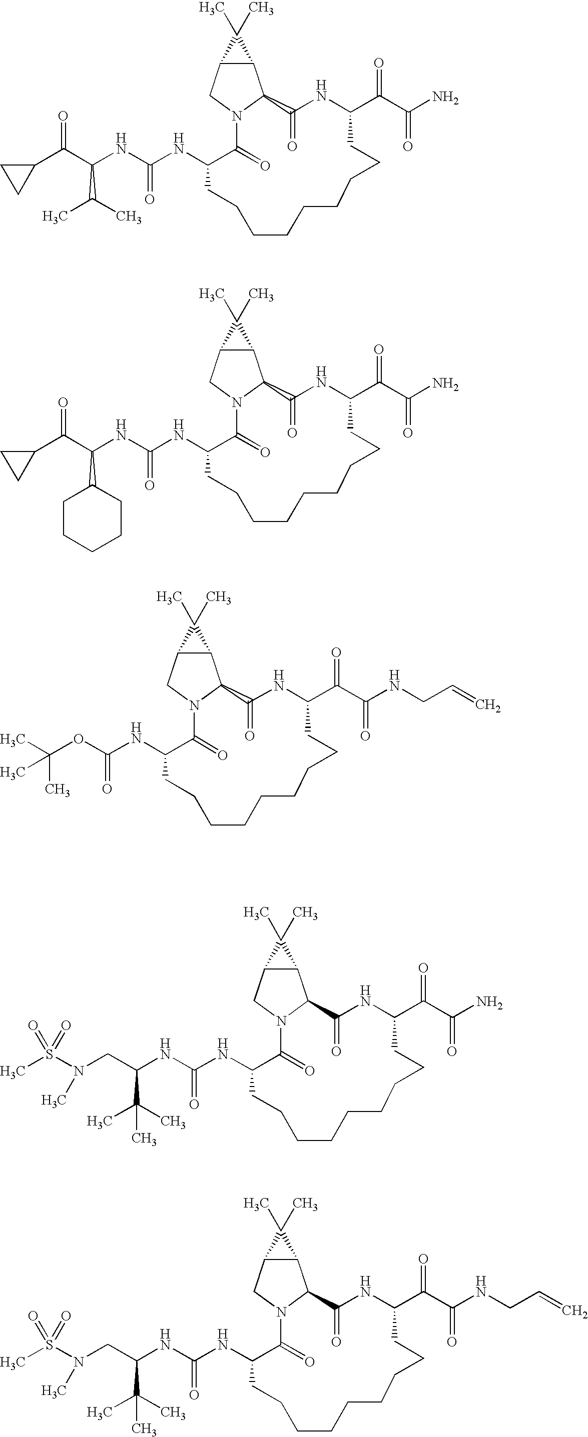 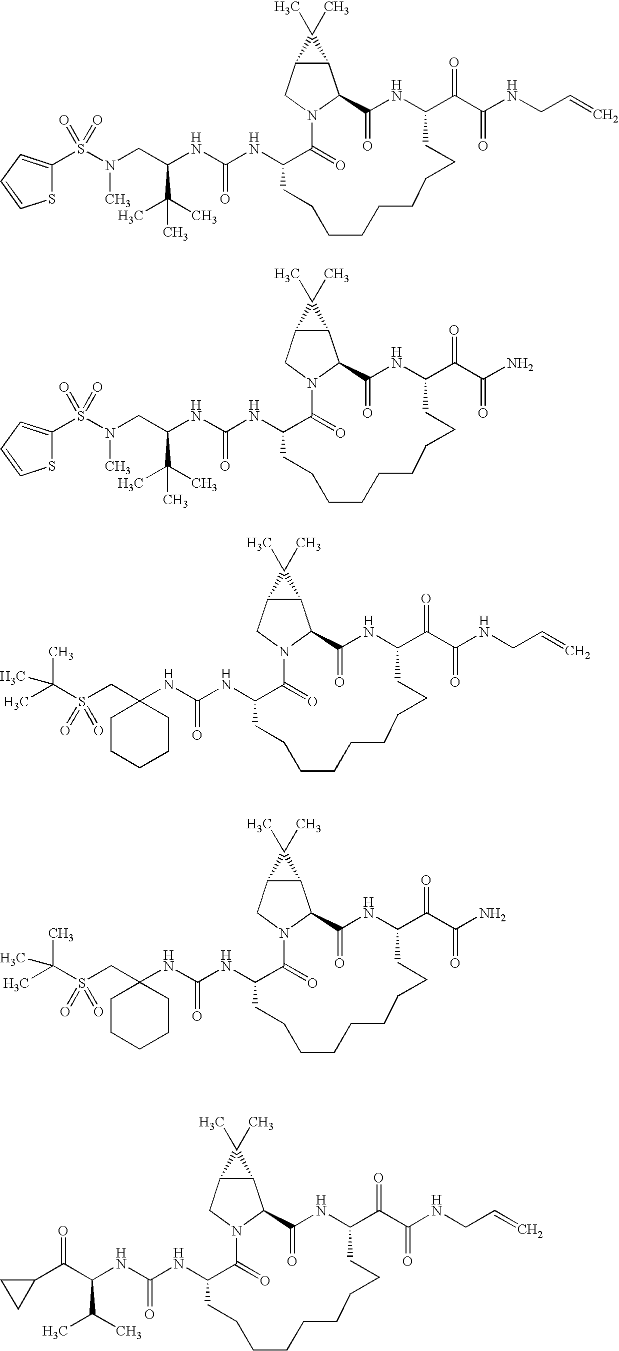 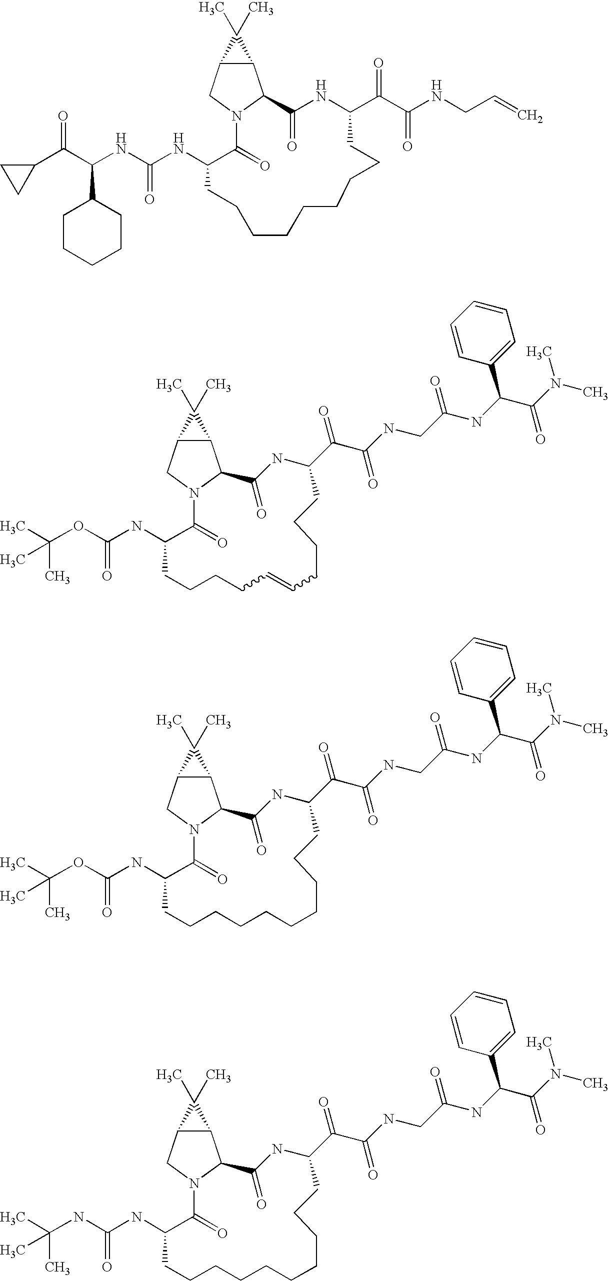 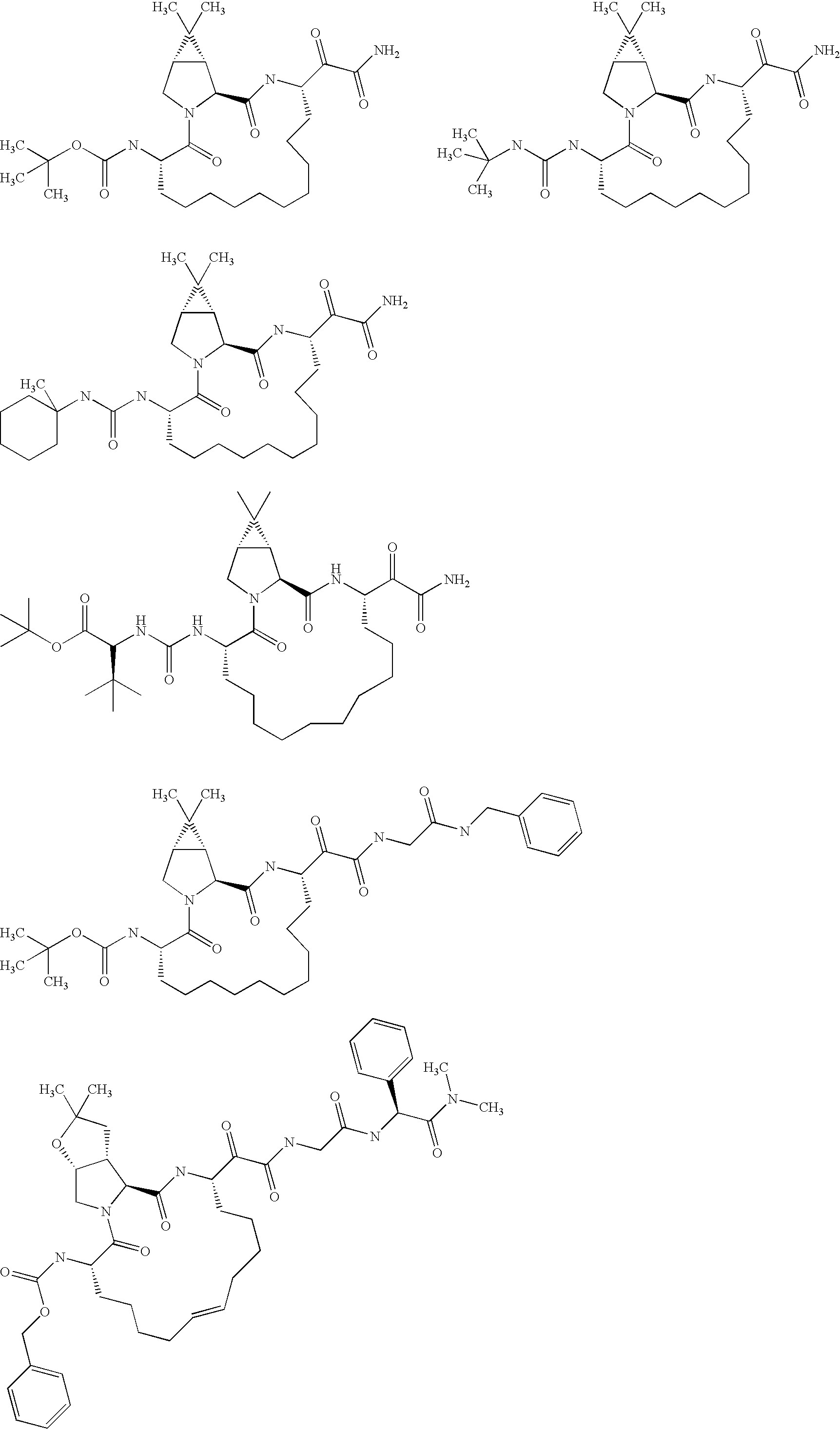 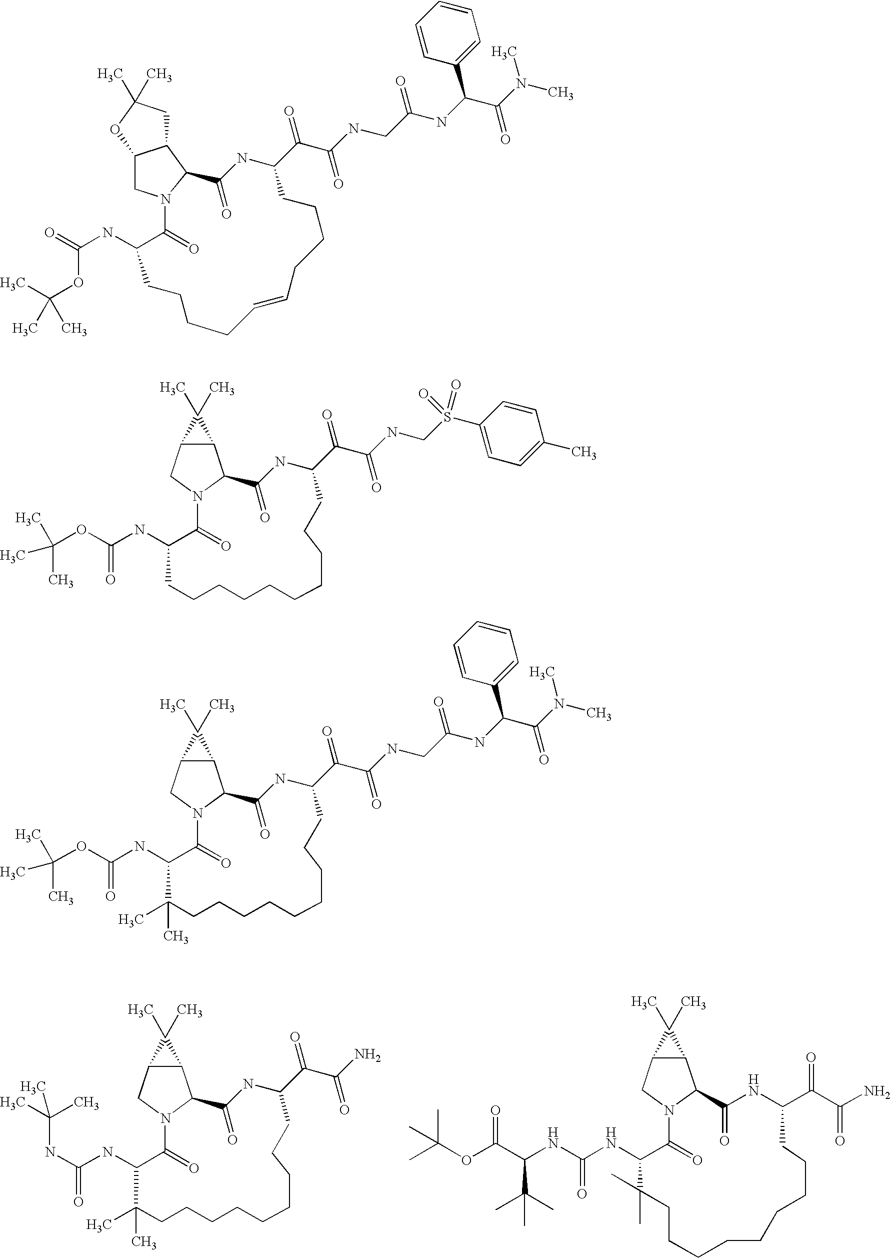 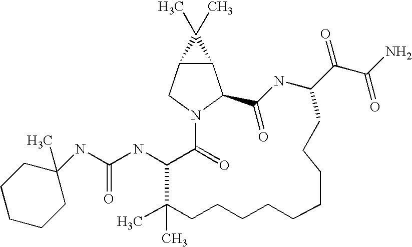
or a pharmaceutically acceptable salt, solvate or ester thereof.

5. A method of inhibiting cathepsin activity in a subject in need thereof comprising administering to said subject an effective amount of at least one compound having the general structure shown in Formula XIII: 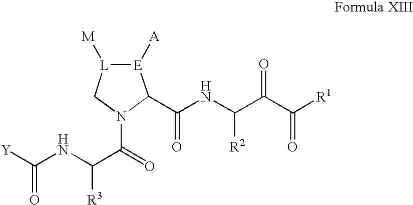
or a pharmaceutically acceptable salt, solvate or ester thereof; wherein:
R¹ is H, OR⁸, NR⁹R¹⁰, or CHR⁹R¹⁰, wherein R⁸, R⁹ and R¹⁰ can be the same or different, each being independently selected from the group consisting of H, alkyl-, alkenyl-, alkynyl-, aryl-, heteroalkyl-, heteroaryl-, cycloalkyl-, heterocyclyl-, arylalkyl-, and heteroarylalkyl;
A and M can be the same or different, each being independently selected from R, OR, NHR, NRR′, SR, SO₂R, and halo; or A and M are connected to each other (in other words, A-E-L-M taken together) such that the moiety: 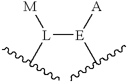
shown above in Formula XIII forms either a three, four, six, seven or eight-membered cycloalkyl, a four to eight-membered heterocyclyl, a six to ten-membered aryl, or a five to ten-membered heteroaryl;
E is C(H) or C(R);
L is C(H), C(R), CH₂C(R), or C(R)CH₂;
R, R′, R², and R³ can be the same or different, each being independently selected from the group consisting of H, alkyl-, alkenyl-, alkynyl-, cycloalkyl-, heteroalkyl-, heterocyclyl-, aryl-, heteroaryl-, (cycloalkyl)alkyl-, (heterocyclyl)alkyl-, aryl-alkyl-, and heteroaryl-alkyl-; or alternately R and R′ in NRR′ are connected to each other such that NRR′ forms a four to eight-membered heterocyclyl;
and Y is selected from the following moieties: 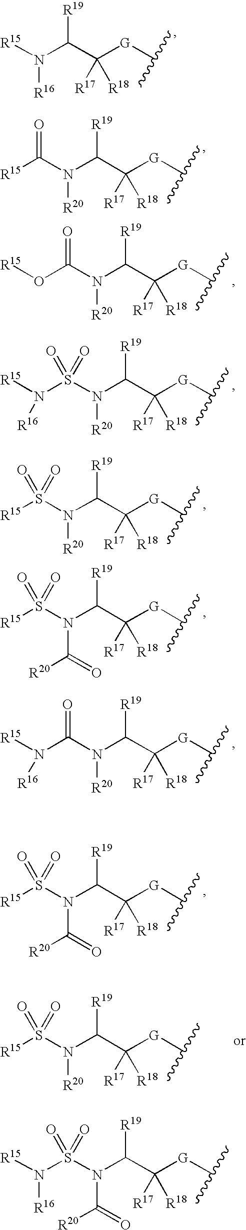
wherein G is NH or O, and R¹⁵, R¹⁶, R¹⁷, R¹⁸, R¹⁹ and R²⁰ can be the same or different, each being independently selected from the group consisting of H, C₁-C₁₀ alkyl, C₁-C₁₀ heteroalkyl, C₂-C₁₀ alkenyl, C₂-C₁₀ heteroalkenyl, C₂-C₁₀ alkynyl, C₂-C₁₀ heteroalkynyl, C₃-C₈ cycloalkyl, C₃-C₈ heterocyclyl, aryl, heteroaryl, or alternately: (i) either R¹⁵ and R¹⁶ can be connected to each other to form a four to eight-membered cycloalkyl or heterocyclyl, or R¹⁵ and R¹⁹ are connected to each other to form a five to eight-membered cycloalkyl or heterocyclyl, or R¹⁵ and R²⁰ are connected to each other to form a five to eight-membered cycloalkyl or heterocyclyl, and (ii) likewise, independently, R¹⁷ and R¹⁸ are connected to each other to form a three to eight-membered cycloalkyl or heterocyclyl,
wherein each of said alkyl, aryl, heteroaryl, cycloalkyl or heterocyclyl can be unsubstituted or optionally independently substituted with one or more moieties selected from the group consisting of: hydroxy, alkoxy, aryloxy, thio, alkylthio, arylthio, amino, amido, alkylamino, arylamino, alkylsulfonyl, arylsulfonyl, sulfonamido, alkylsulfonamido, arylsulfonamido, keto, carboxy, carbalkoxy, carboxamido, alkoxycarbonylamino, alkoxycarbonyloxy, alkylureido, arylureido, halo, cyano, and nitro.

6. The method of claim 5, wherein said compound is selected from the group consisting of: 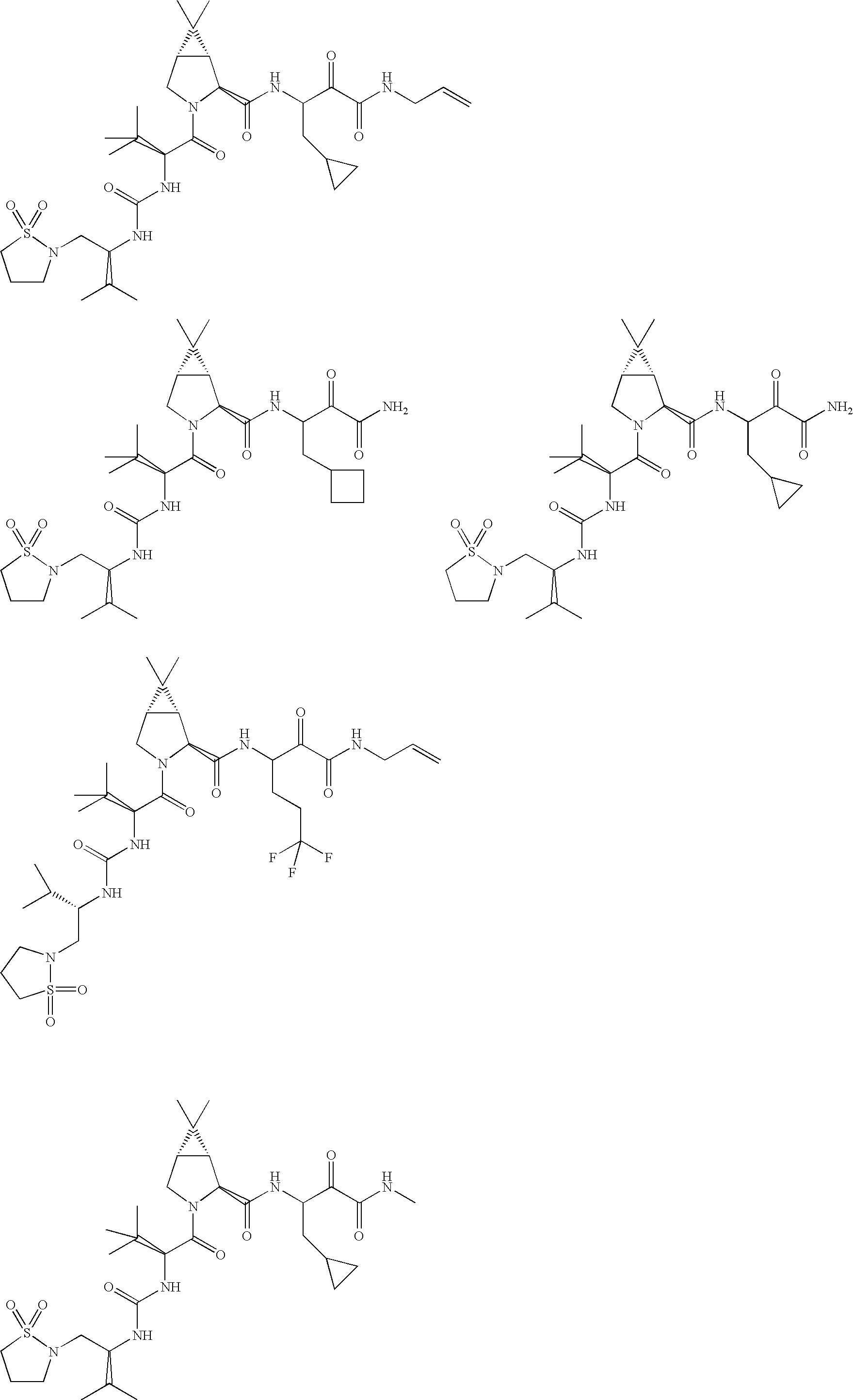 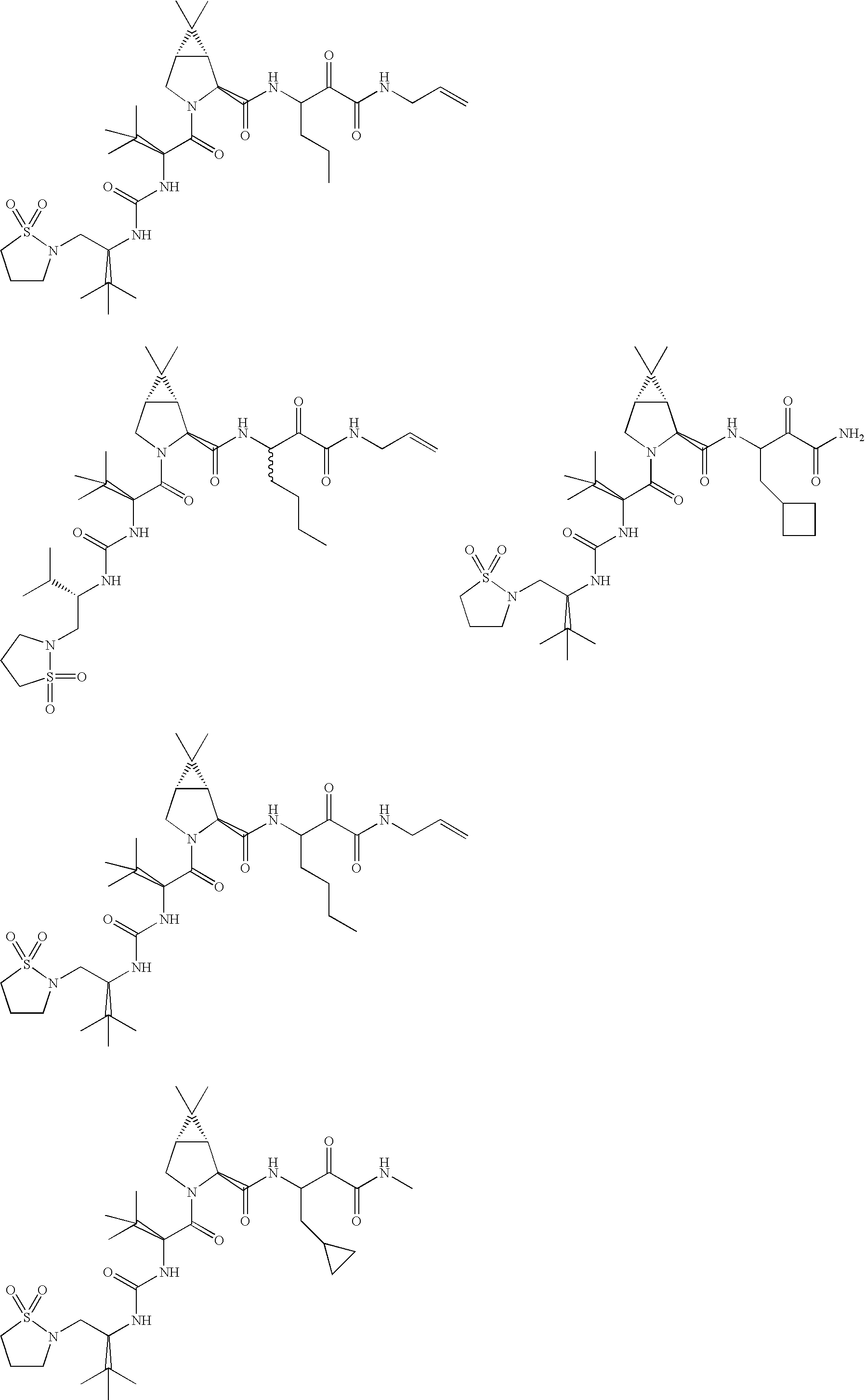 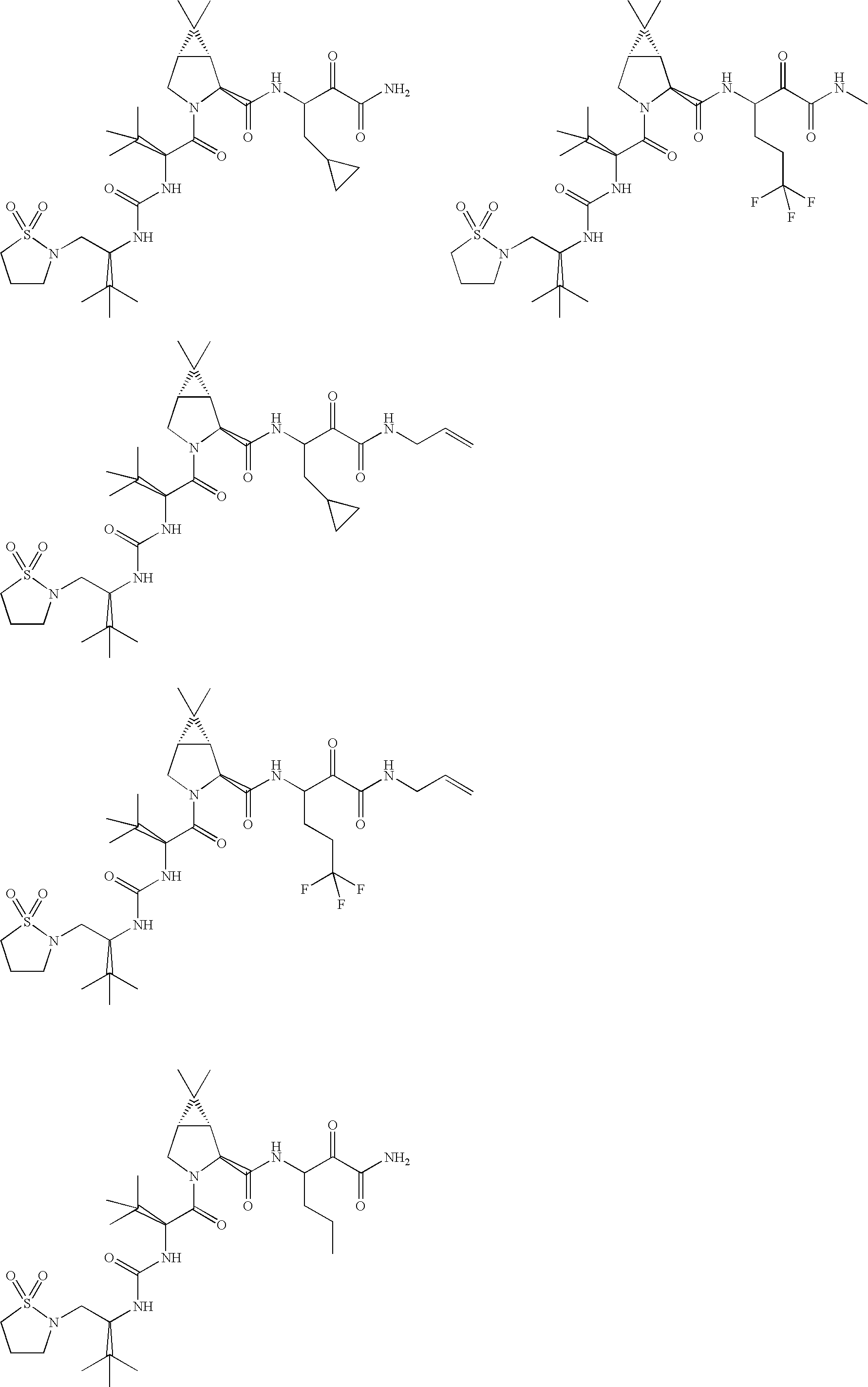 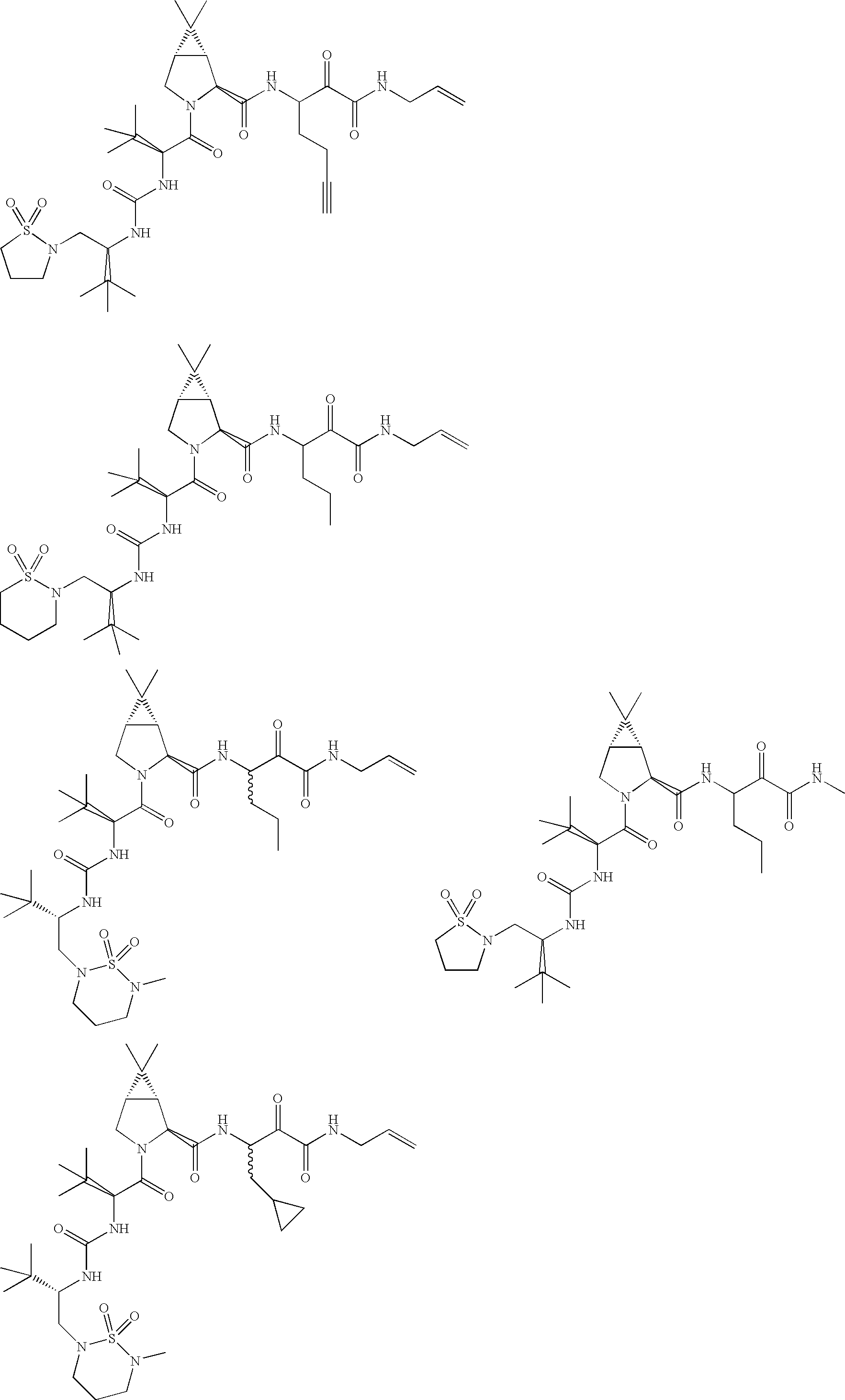 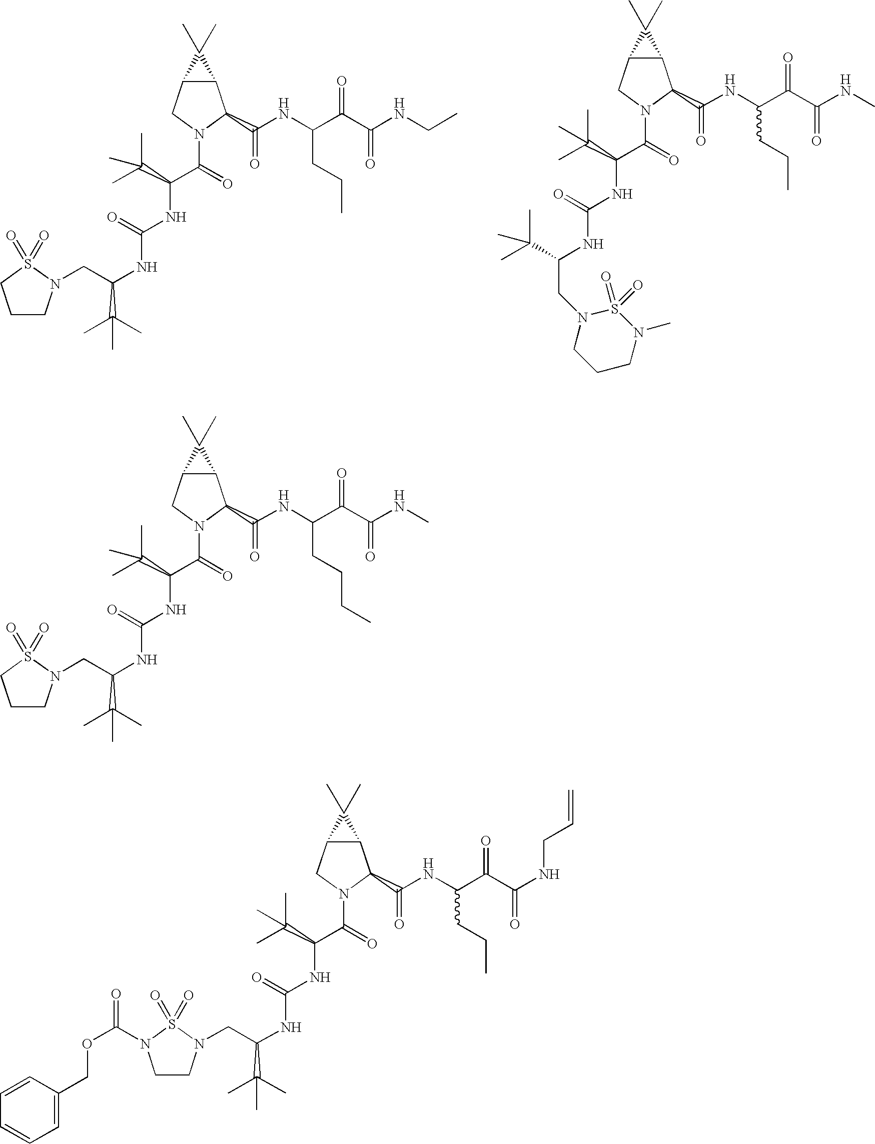 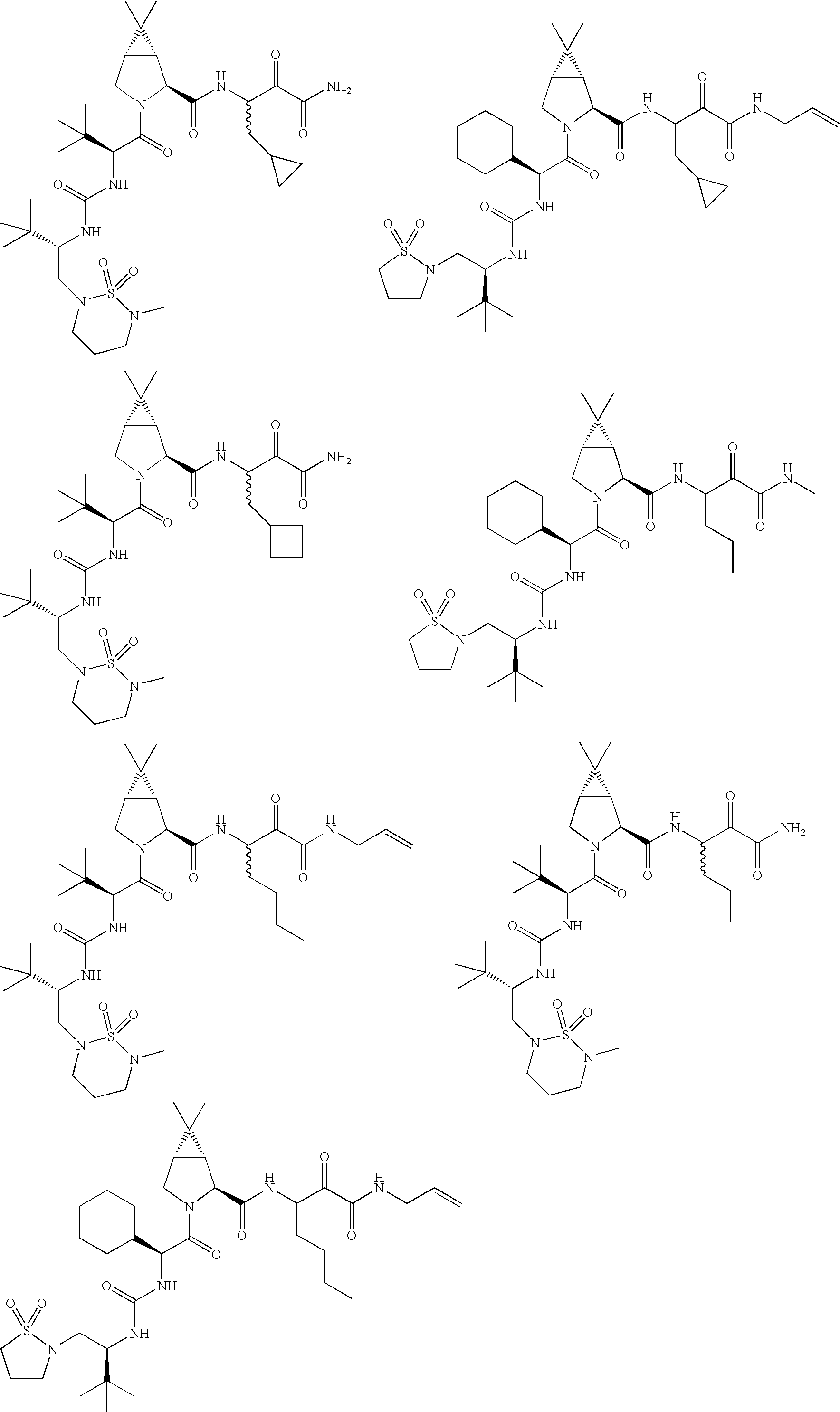 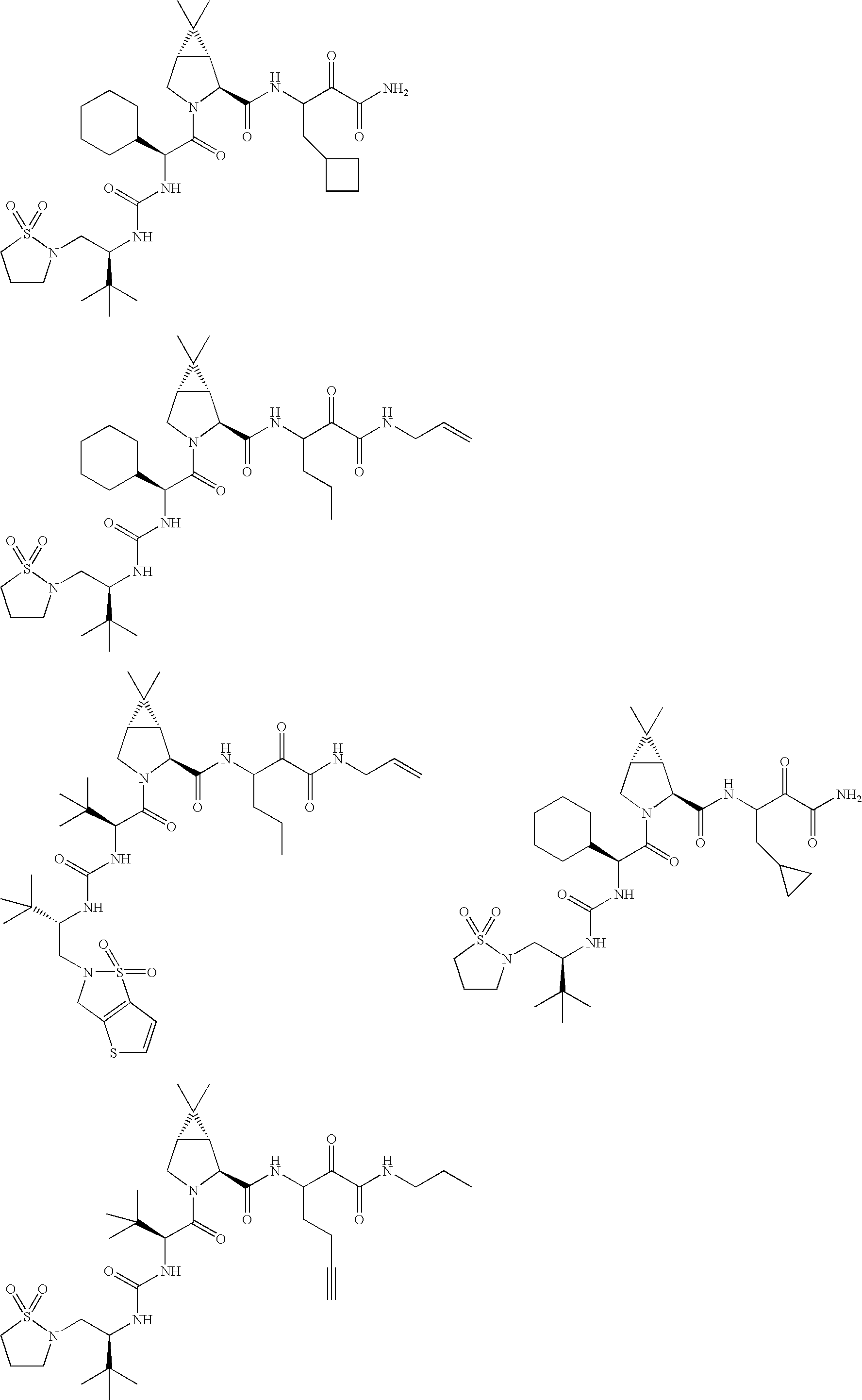 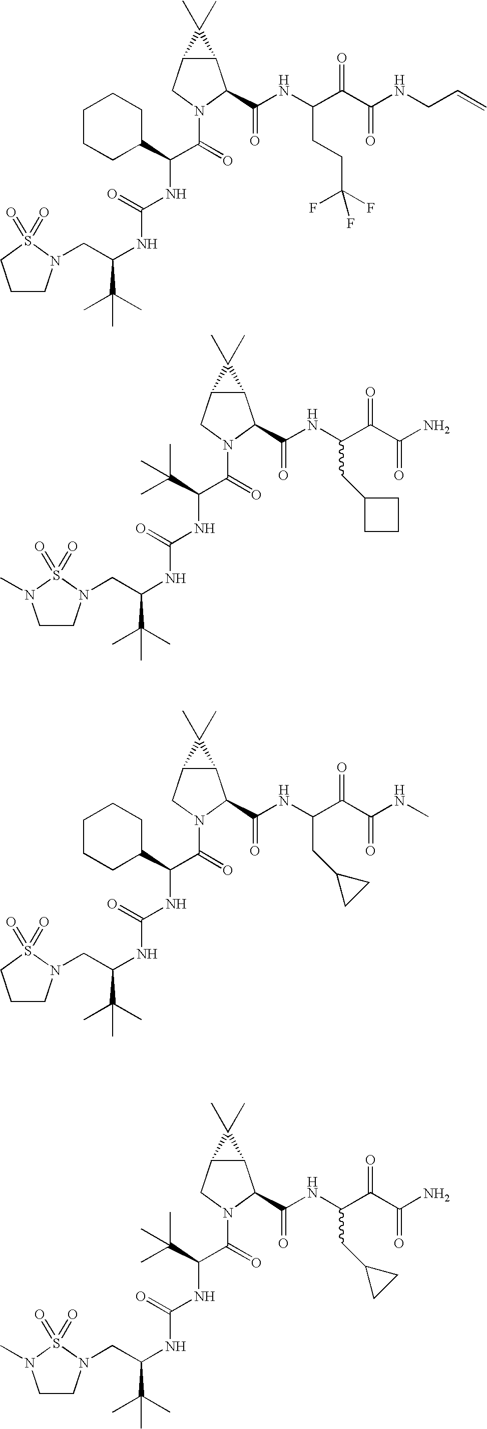 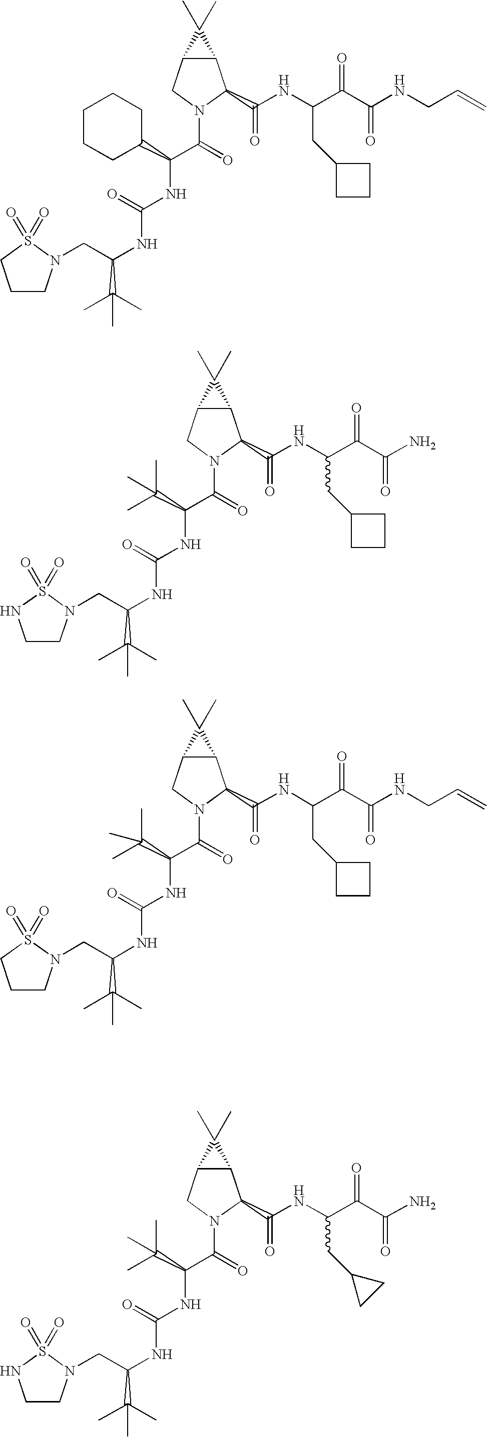 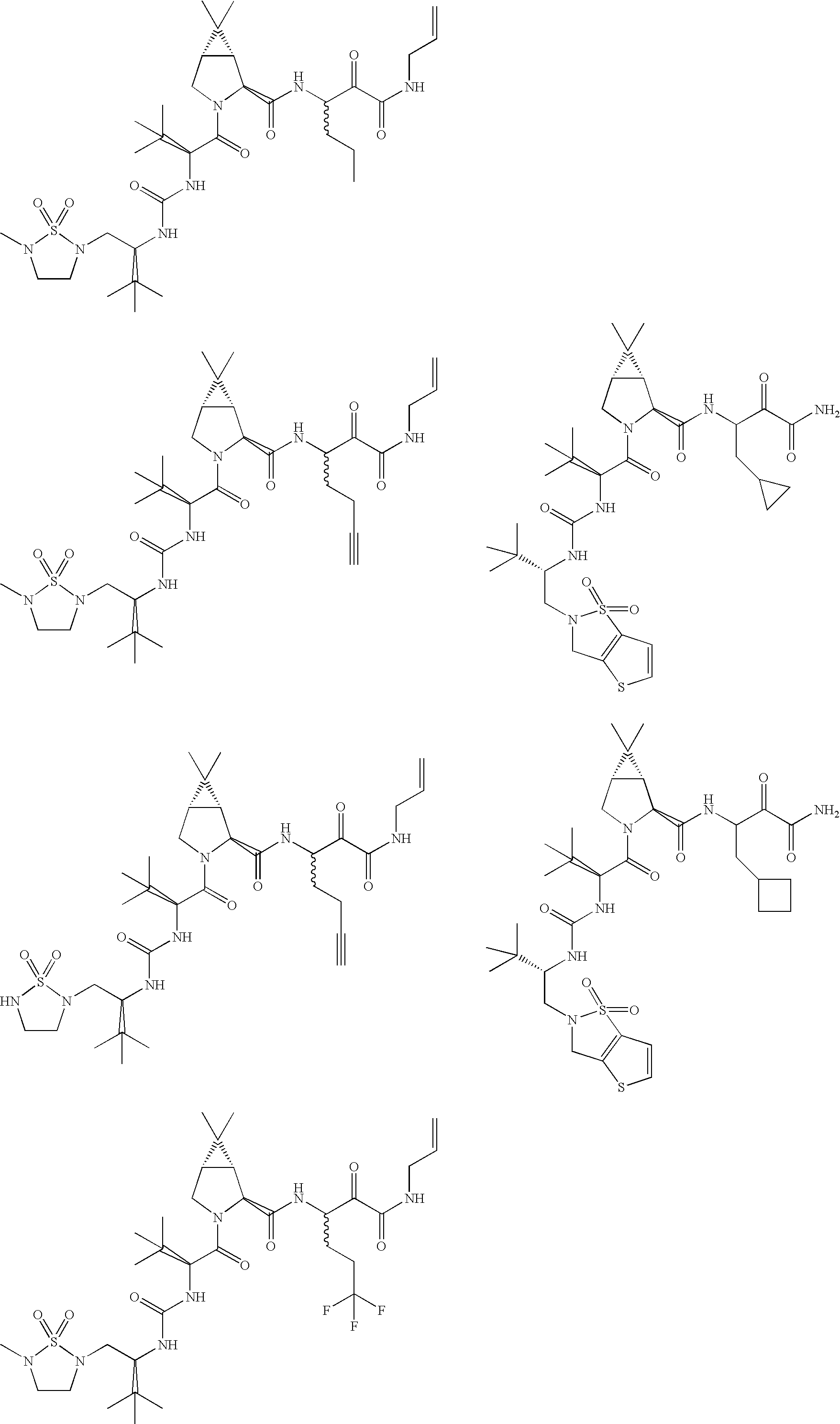 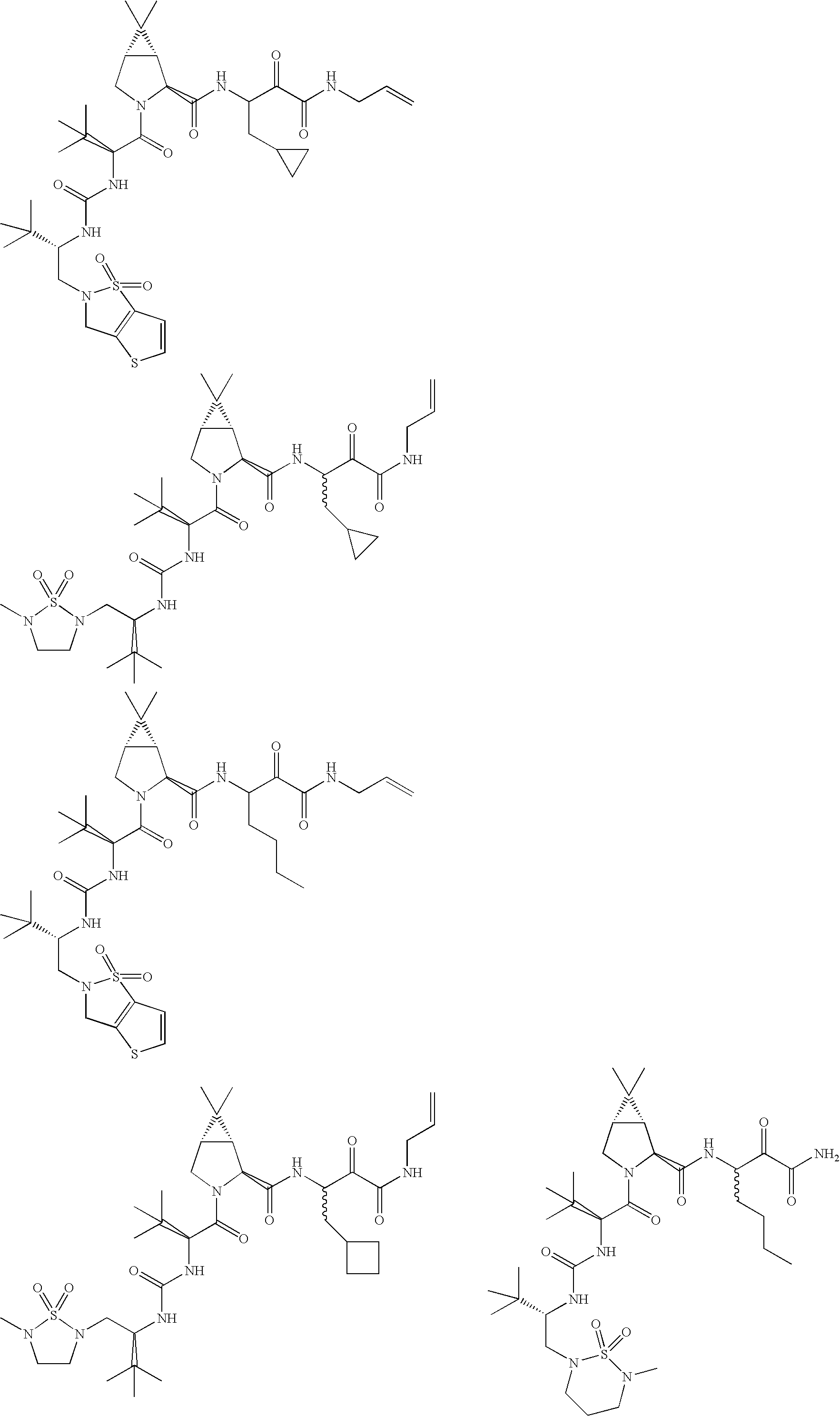 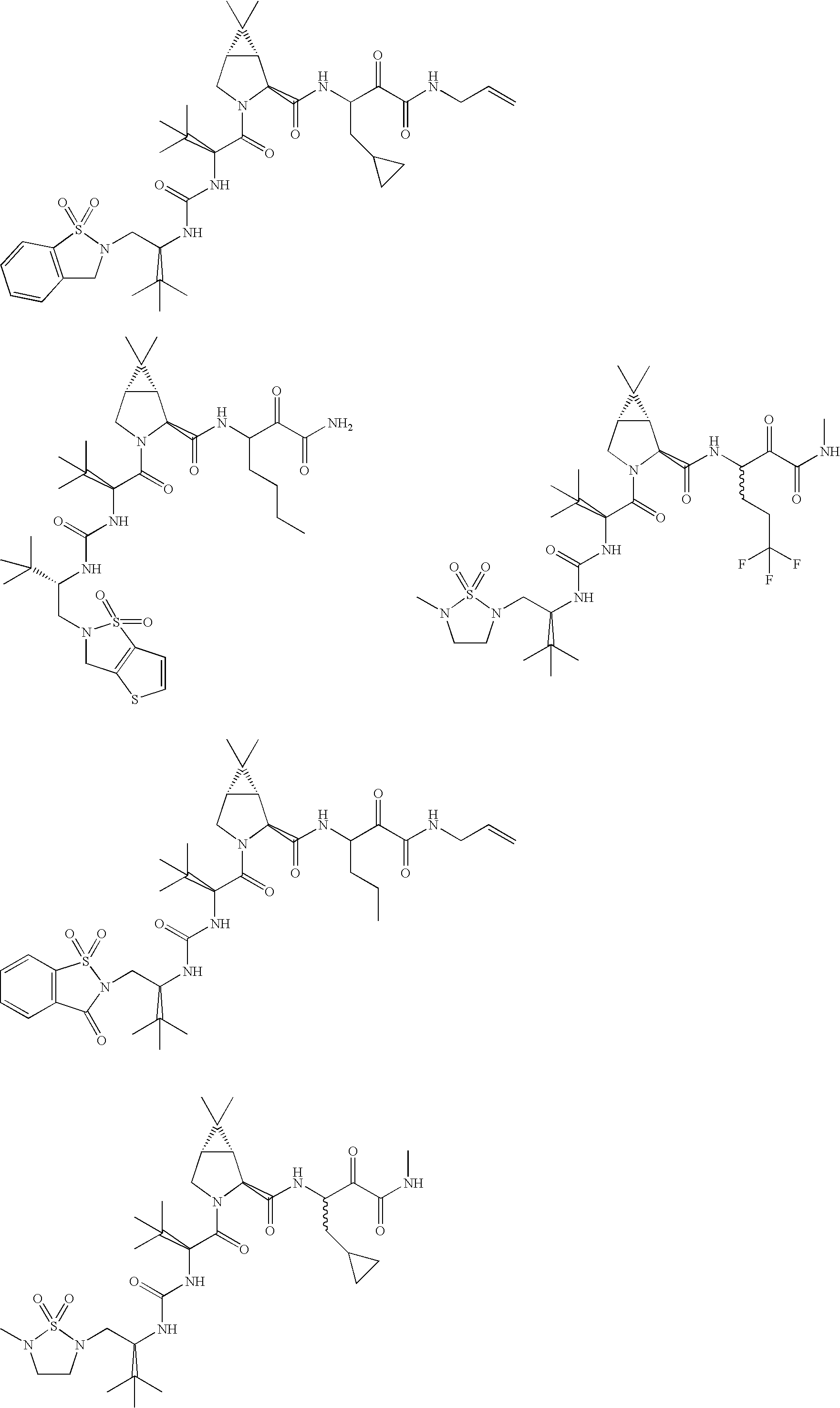 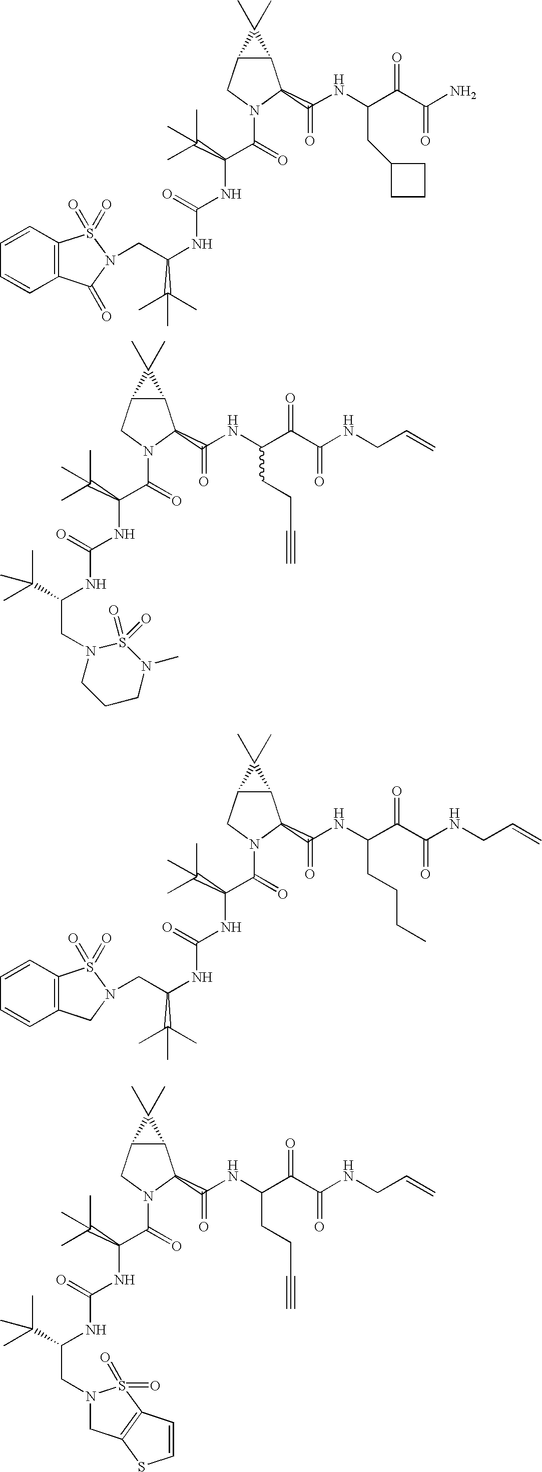 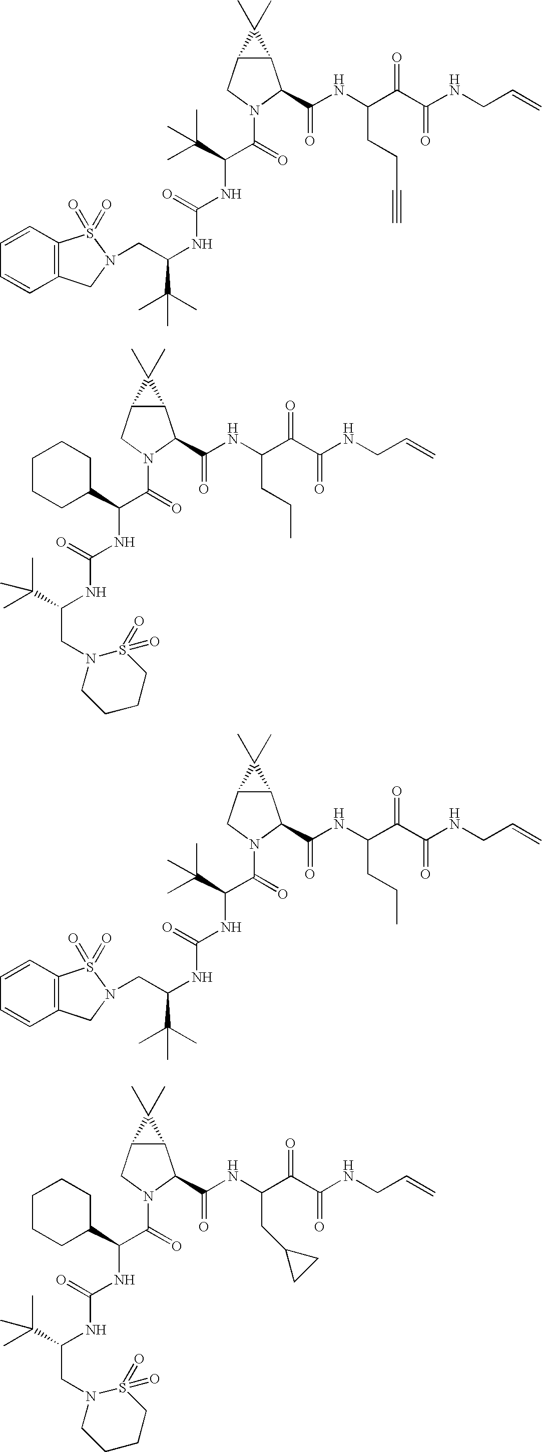 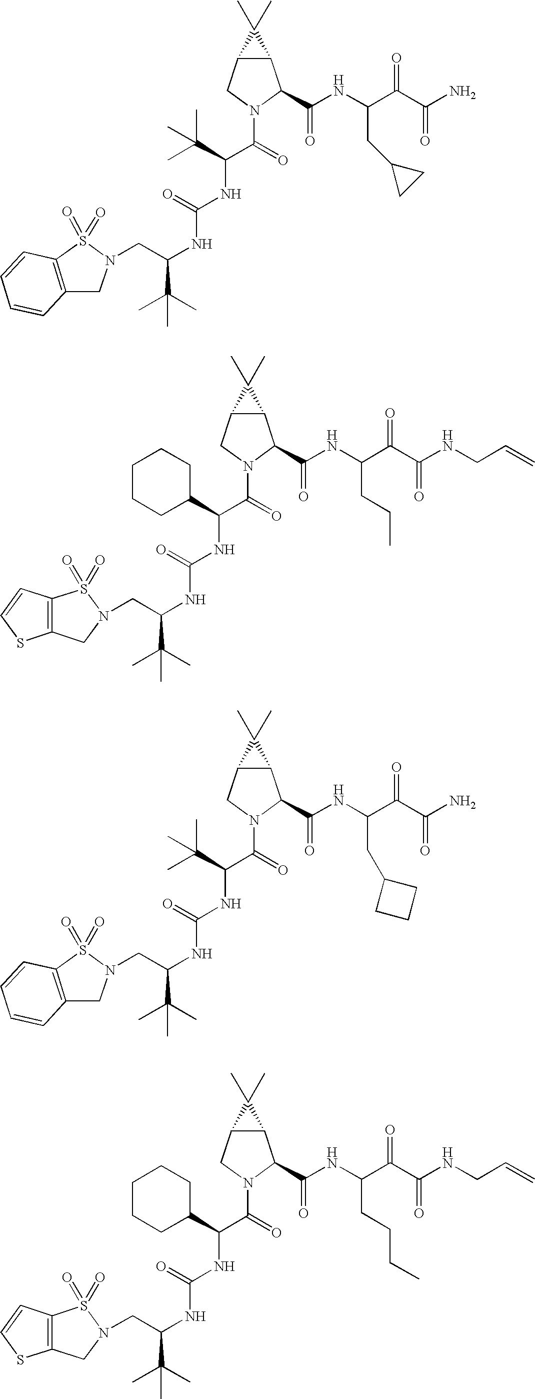 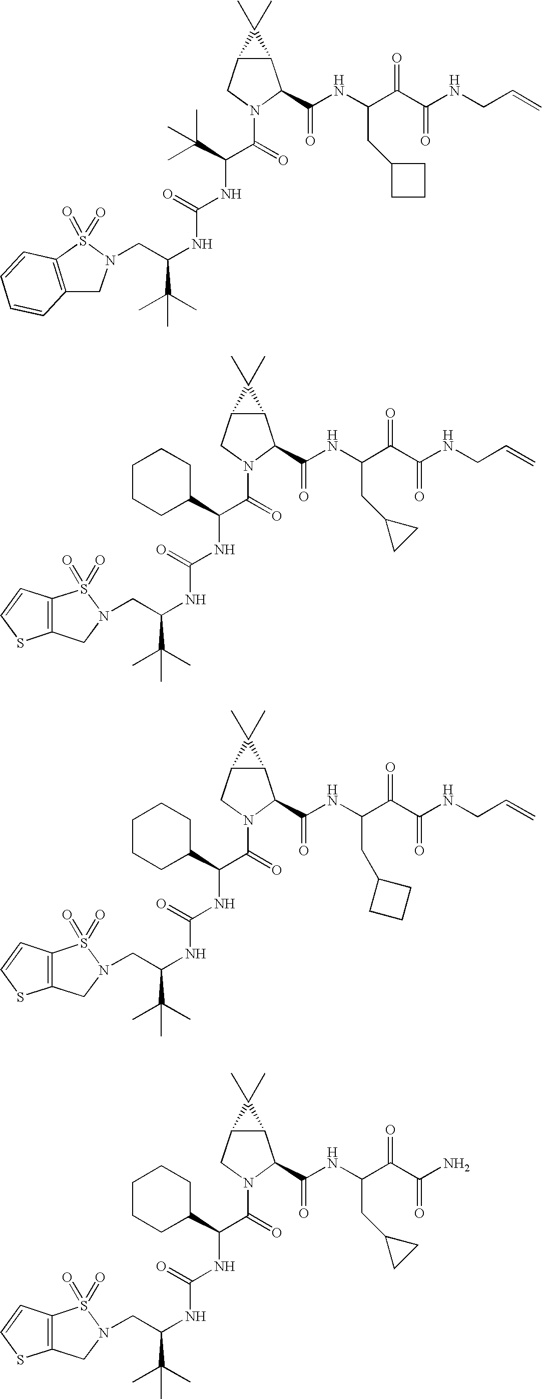 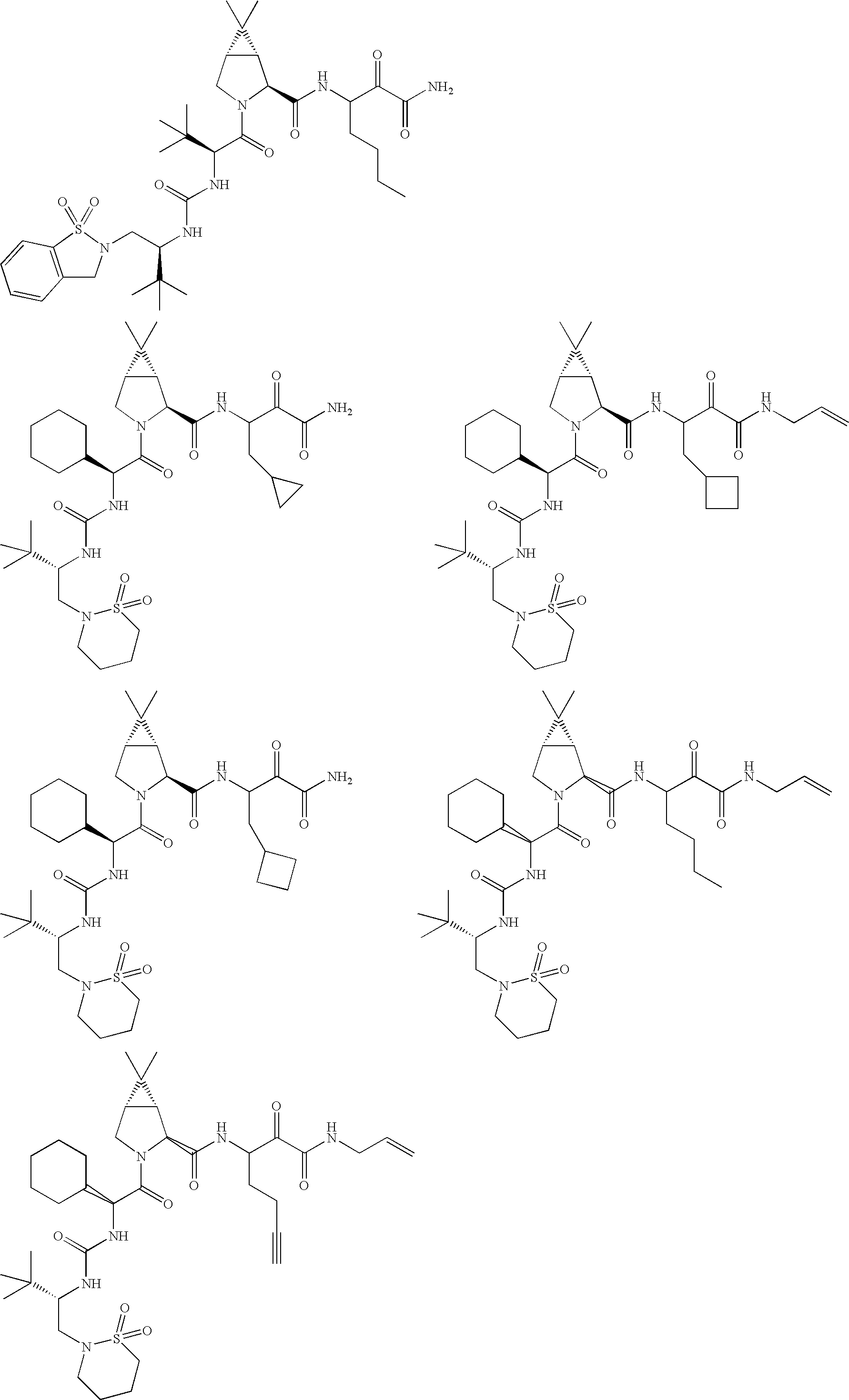 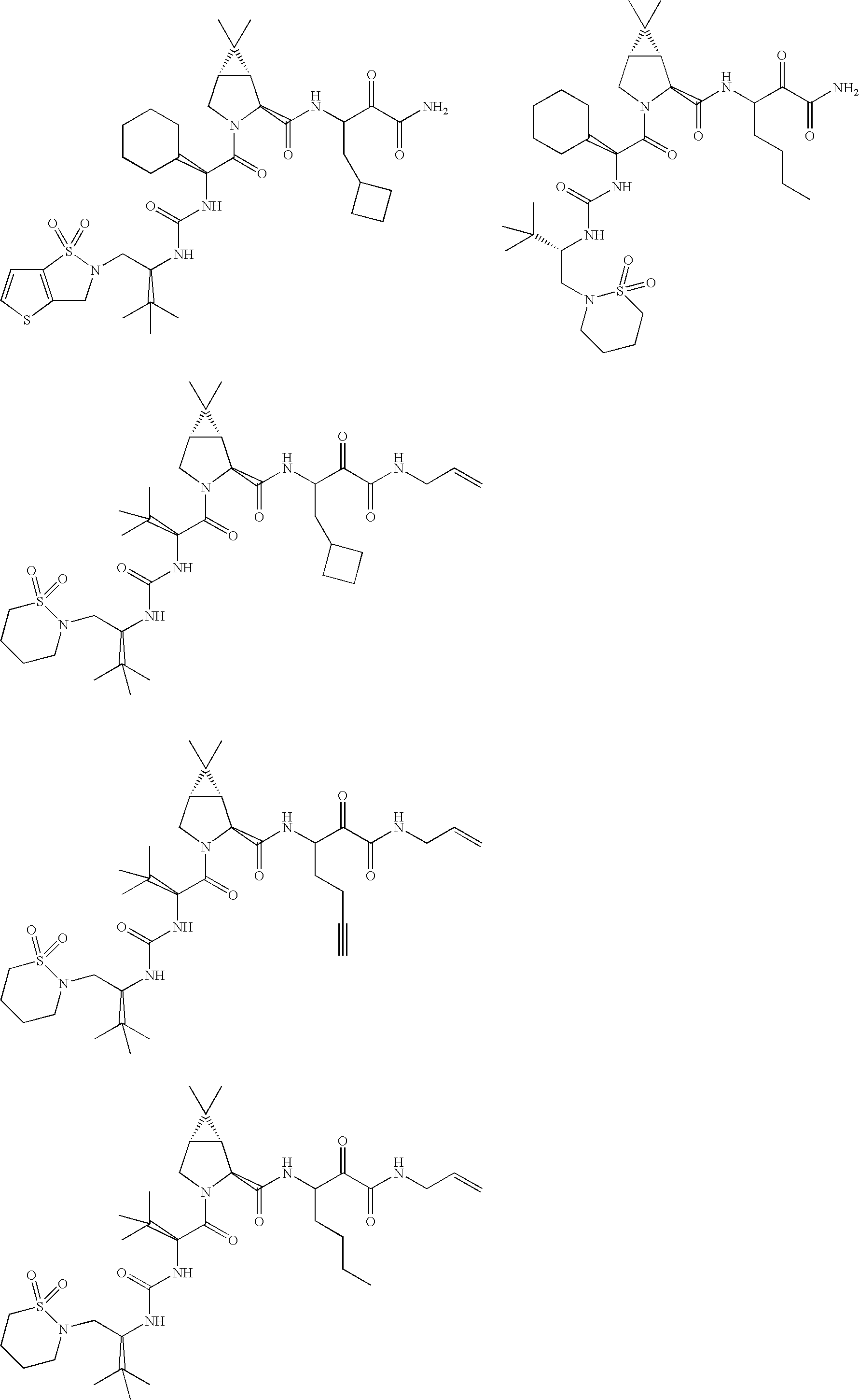 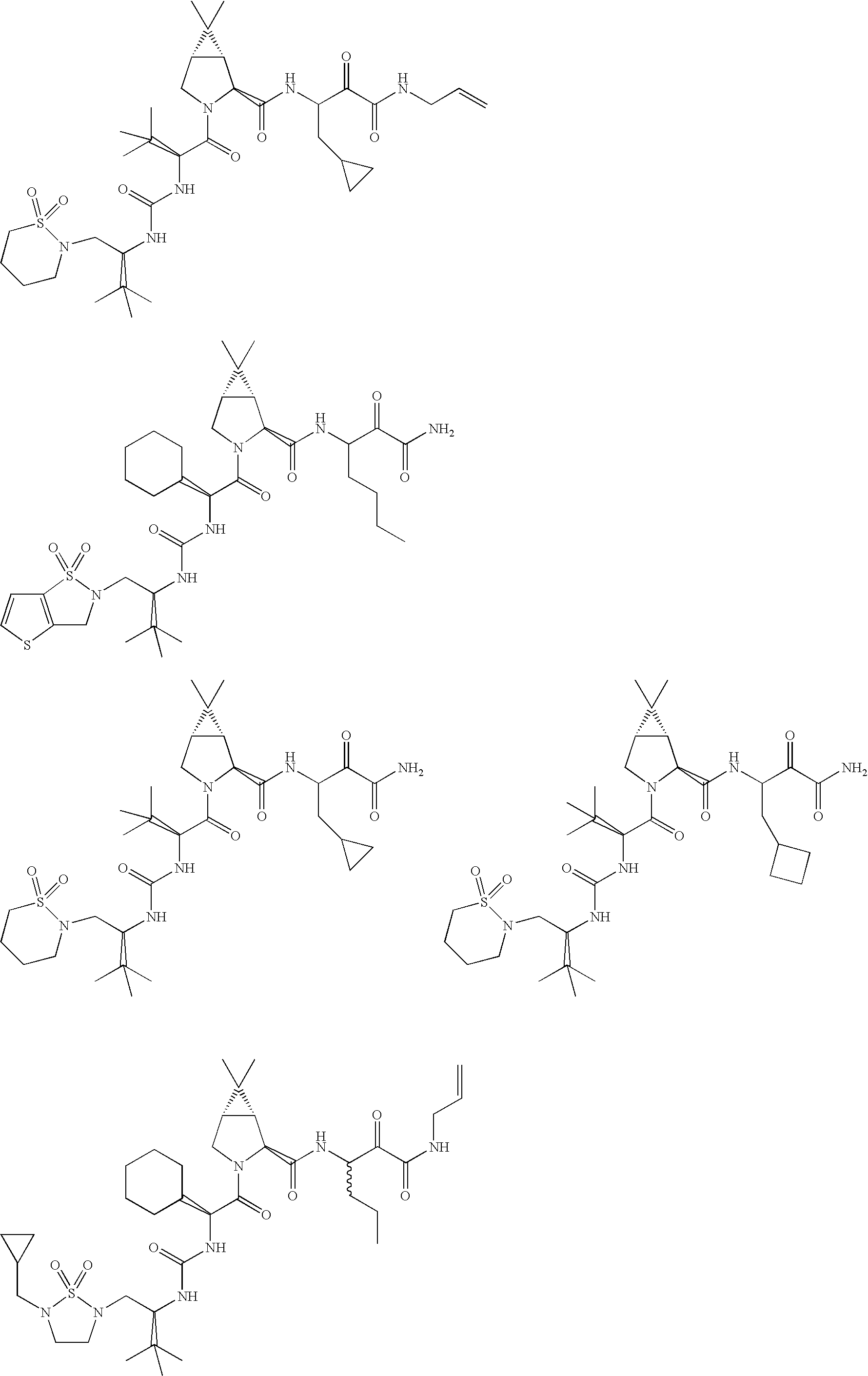 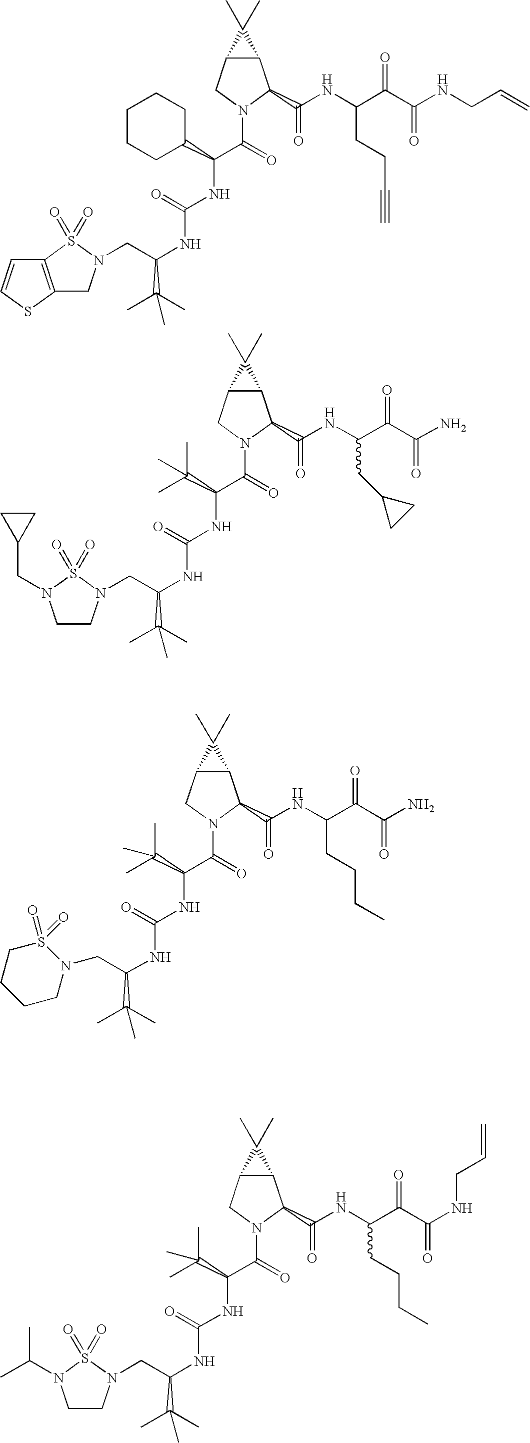 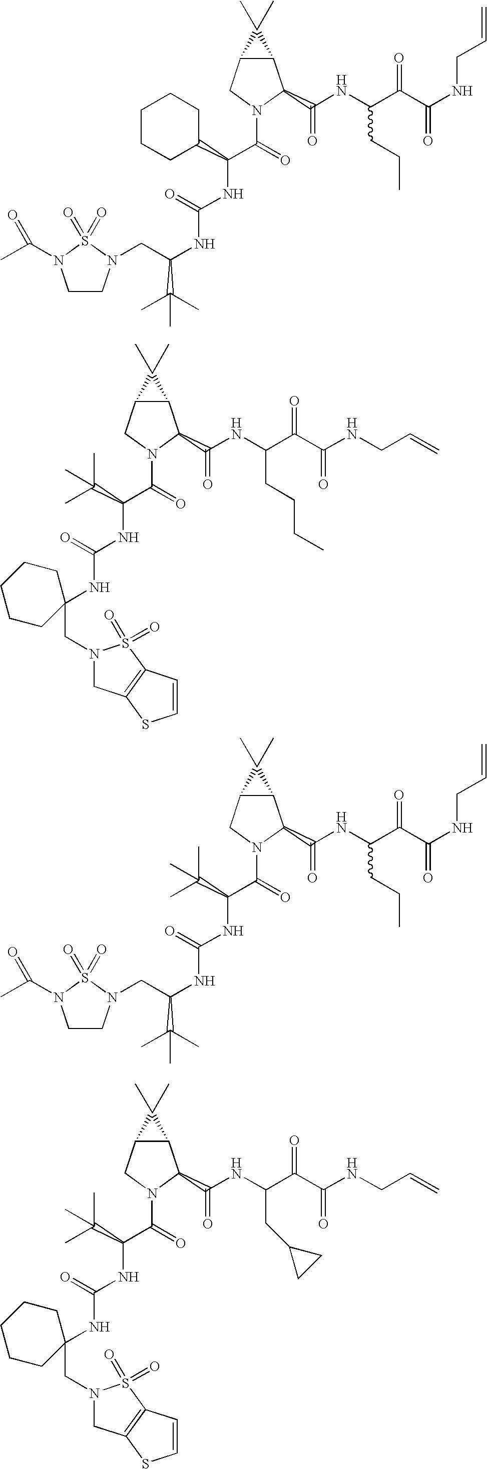 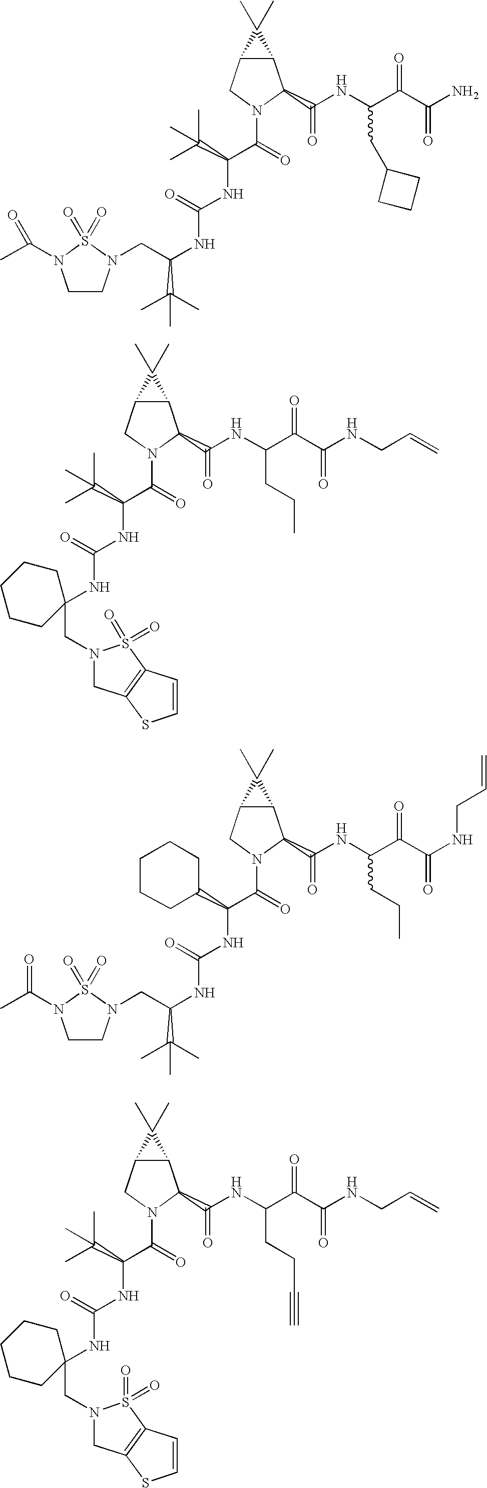 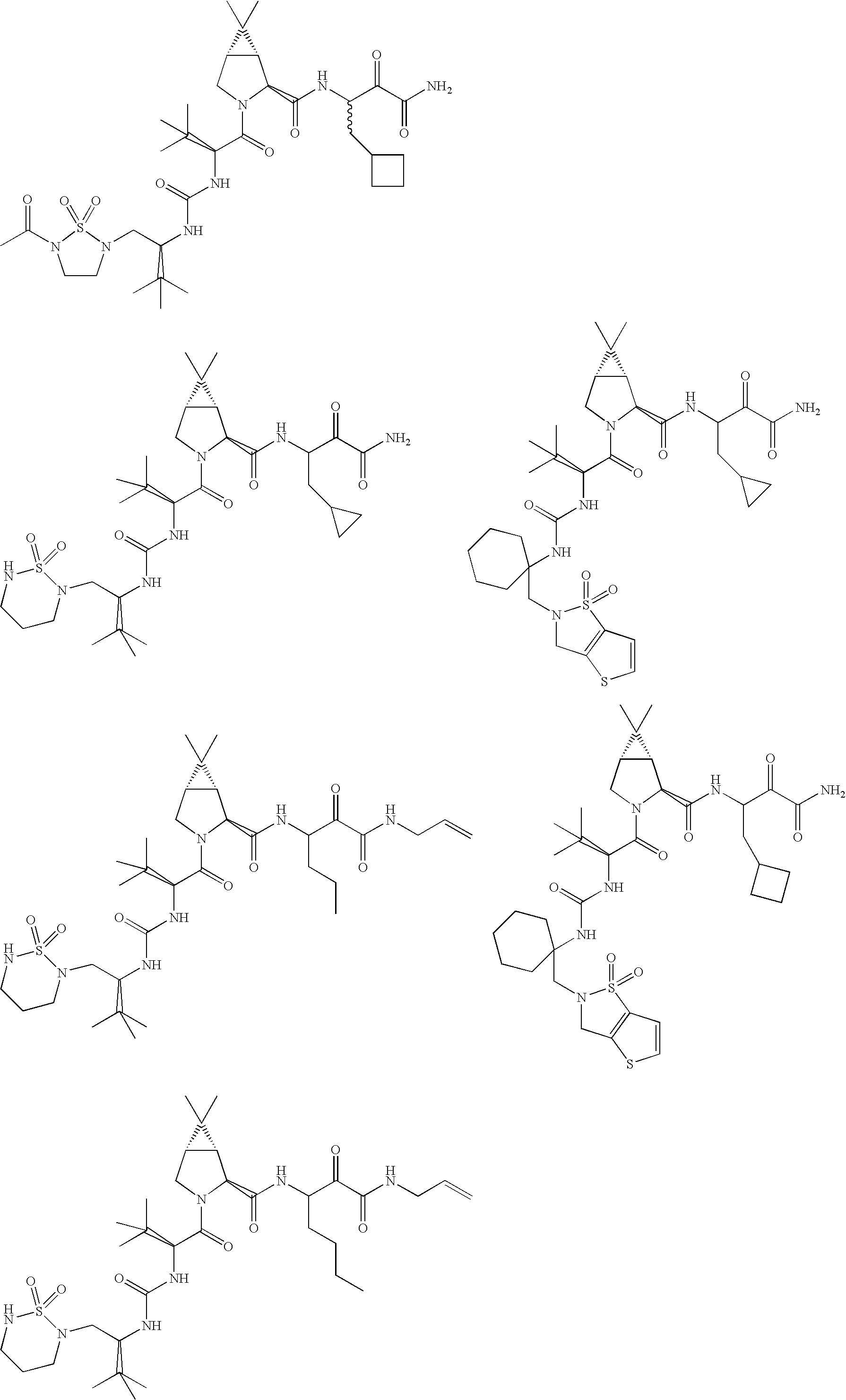 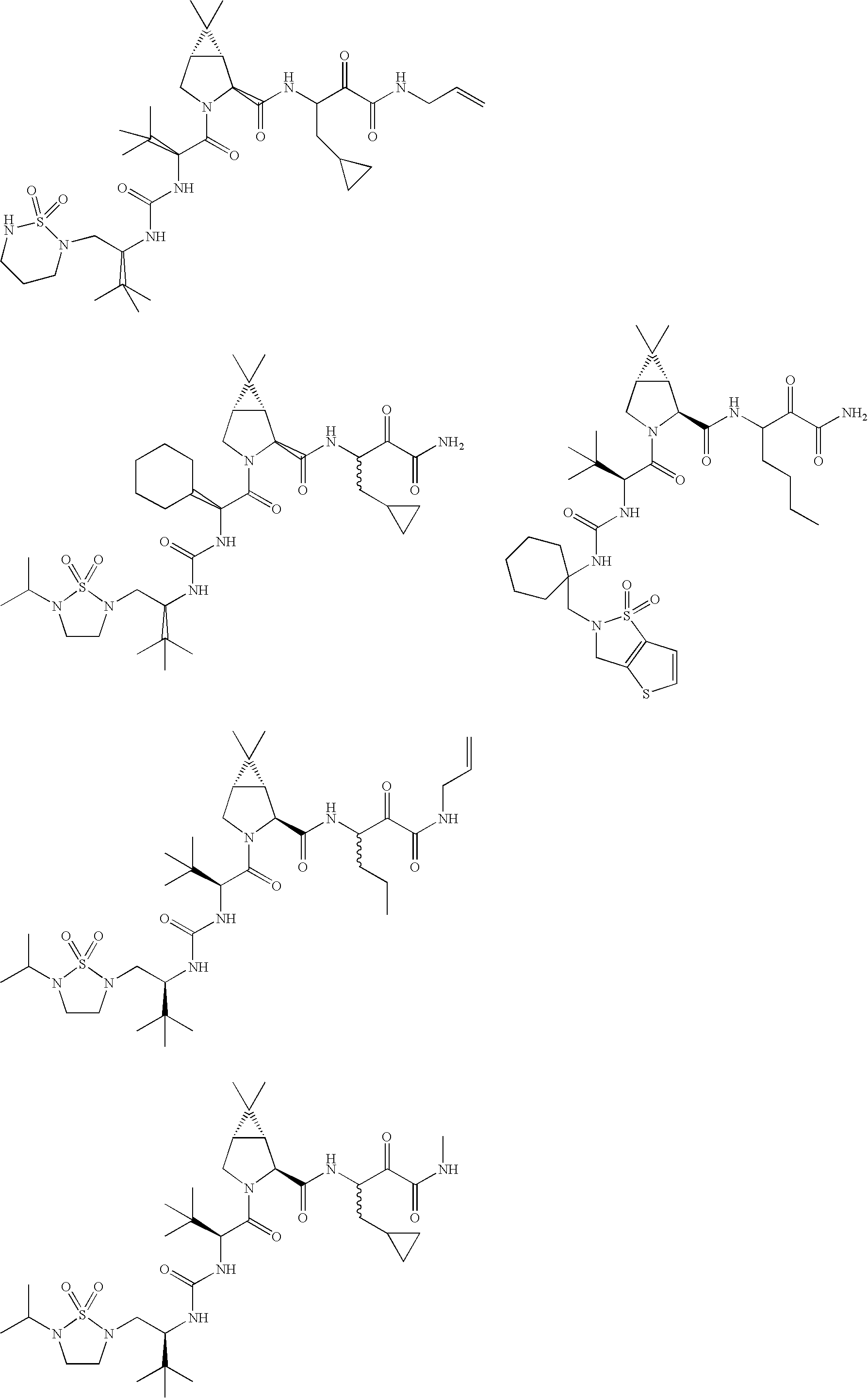 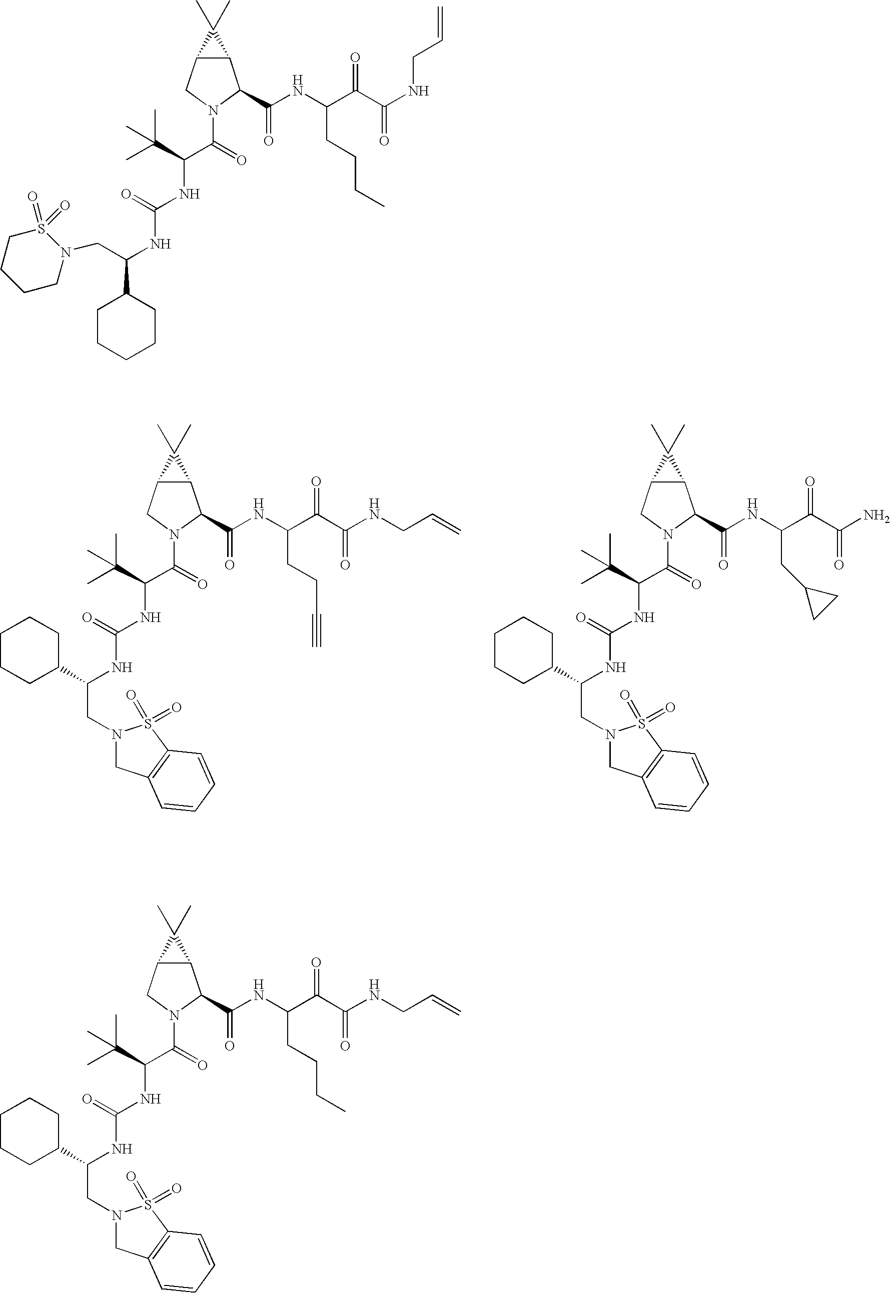 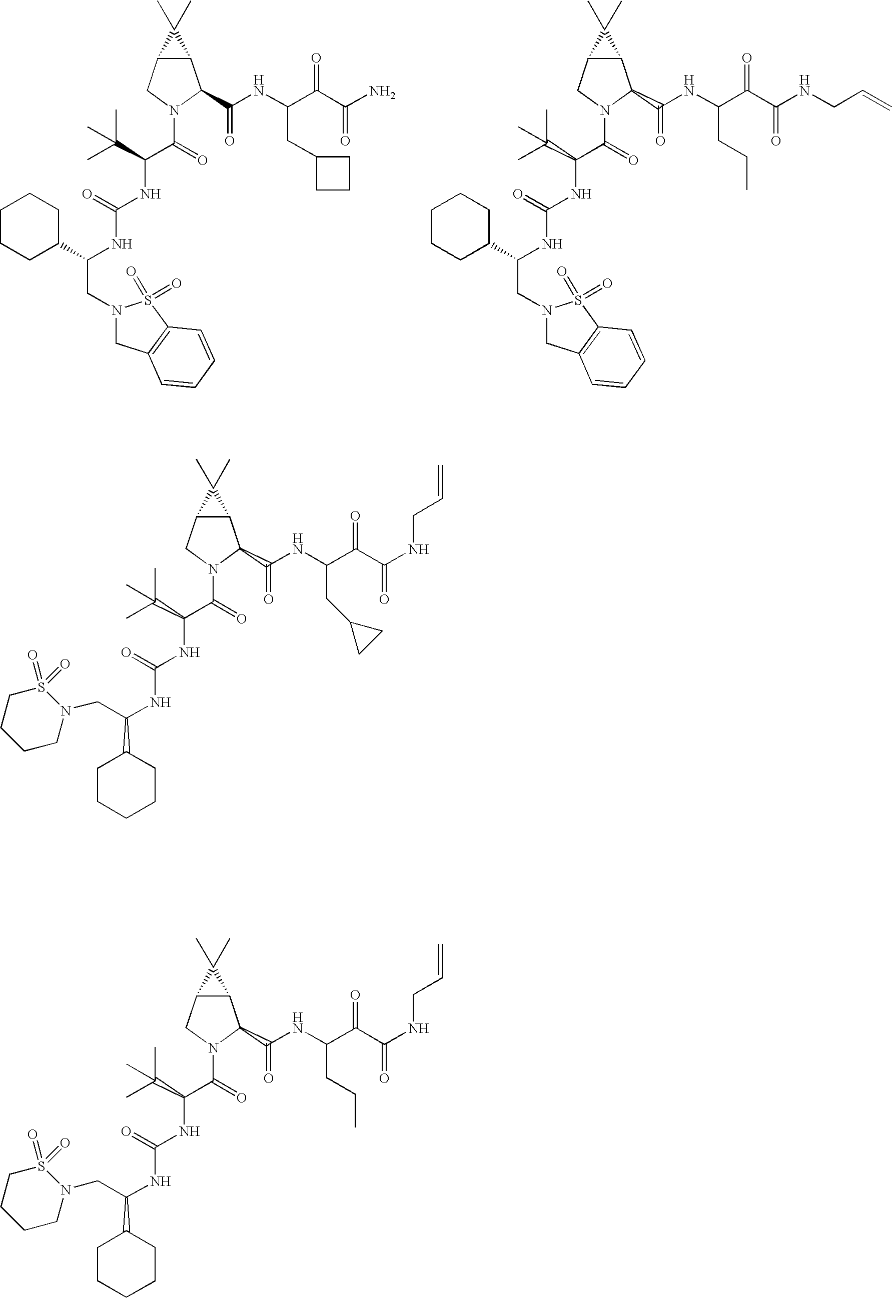 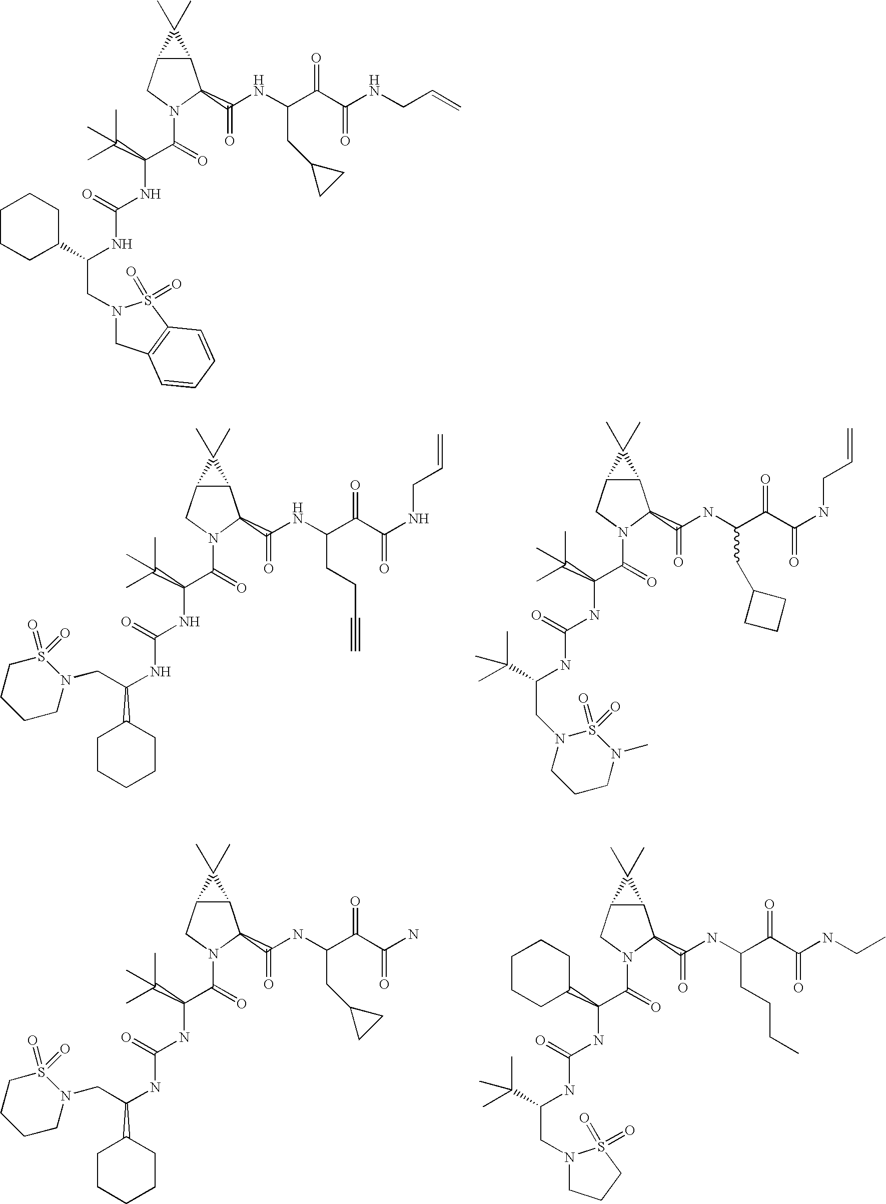 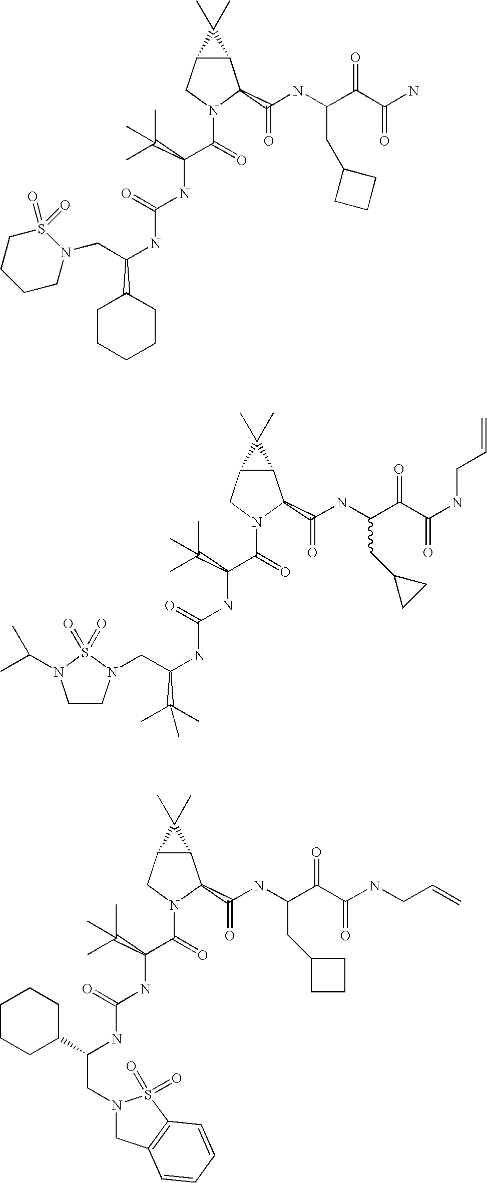 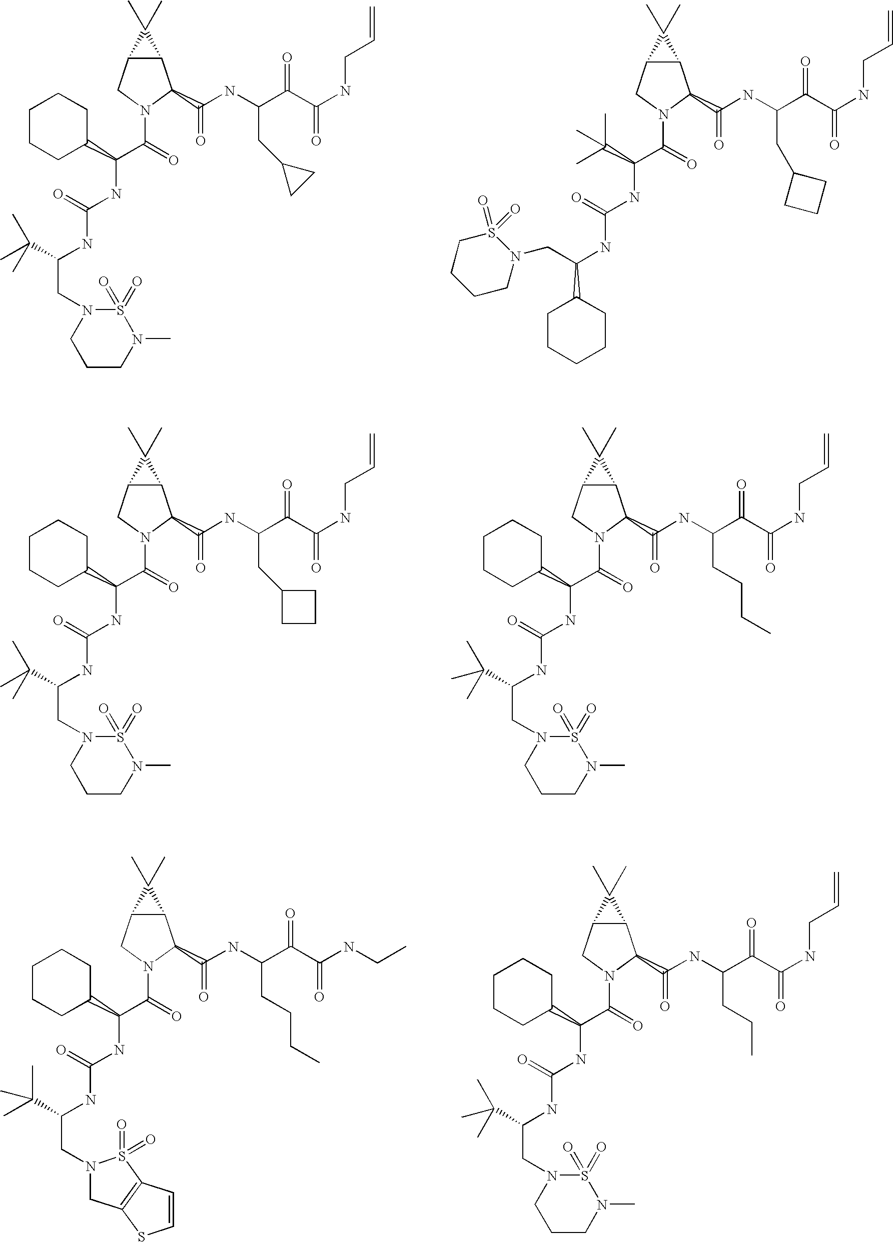 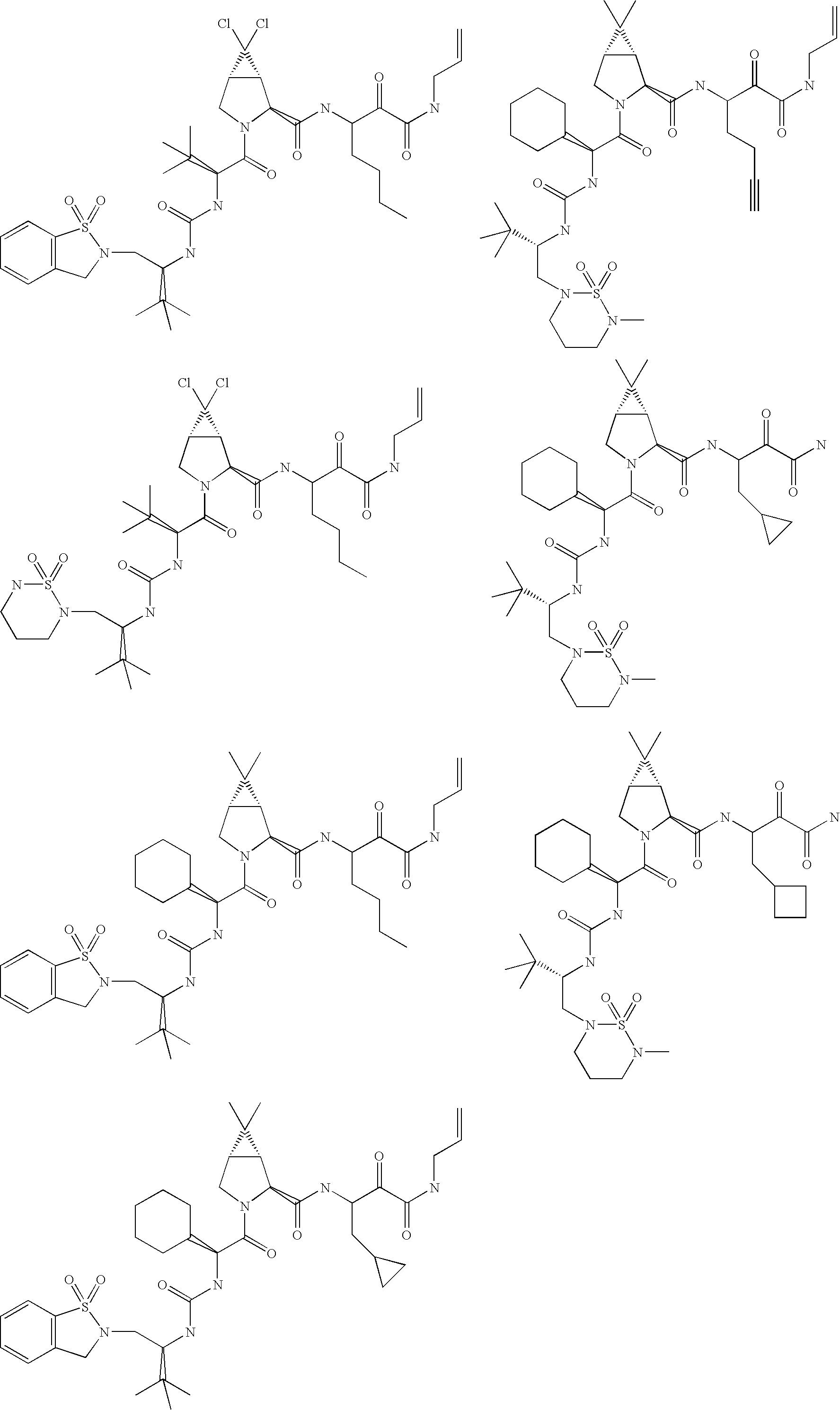 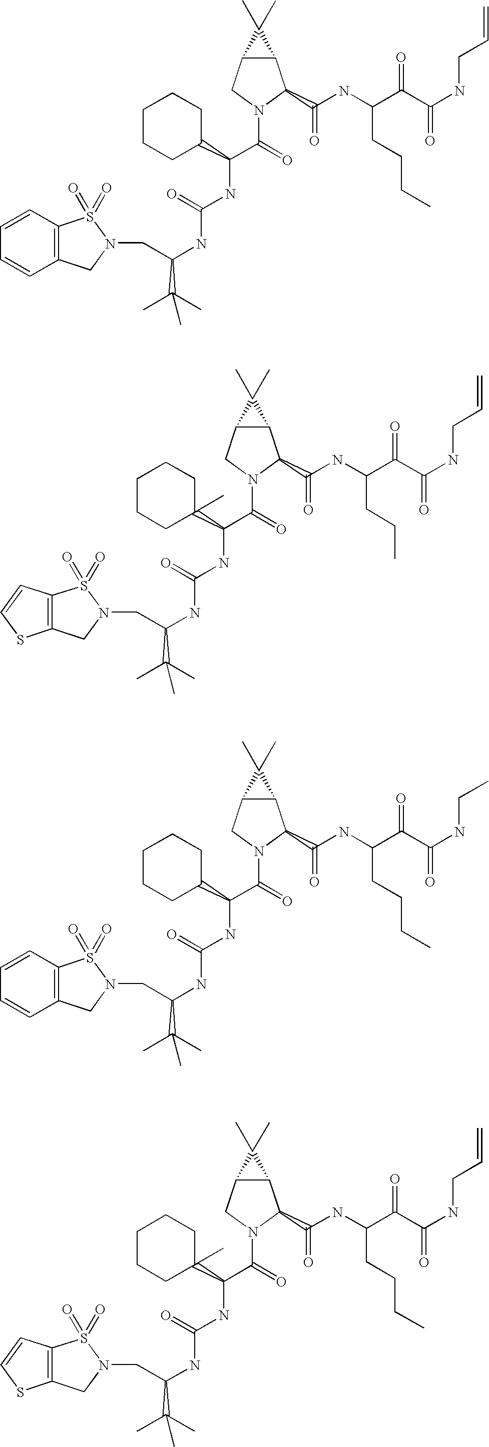 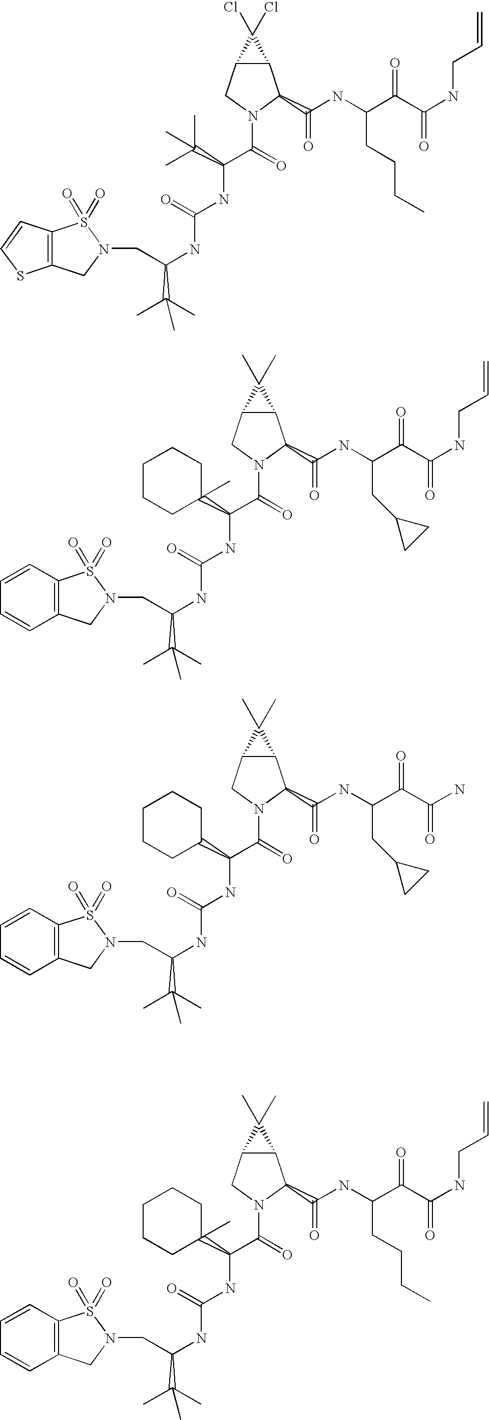 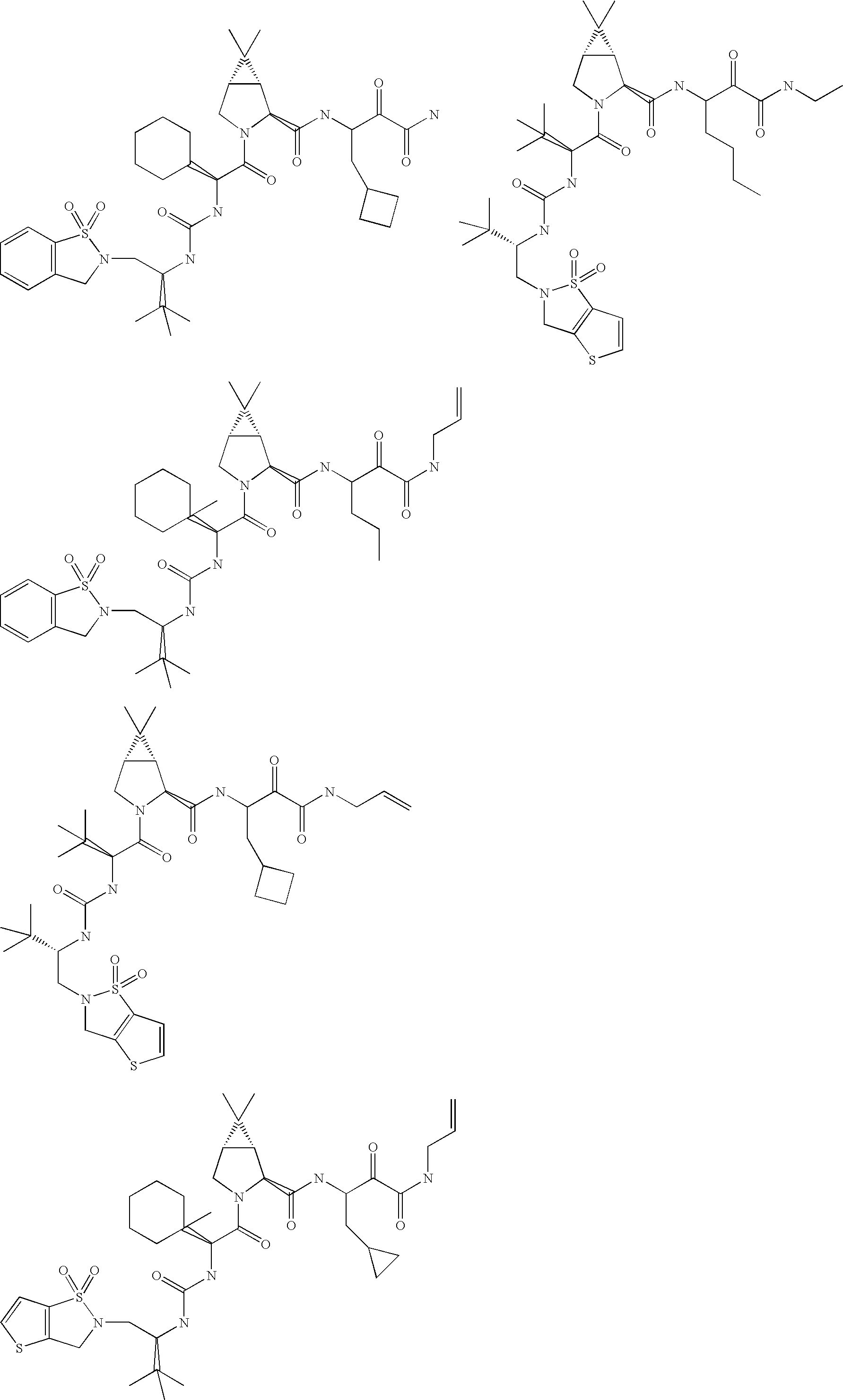 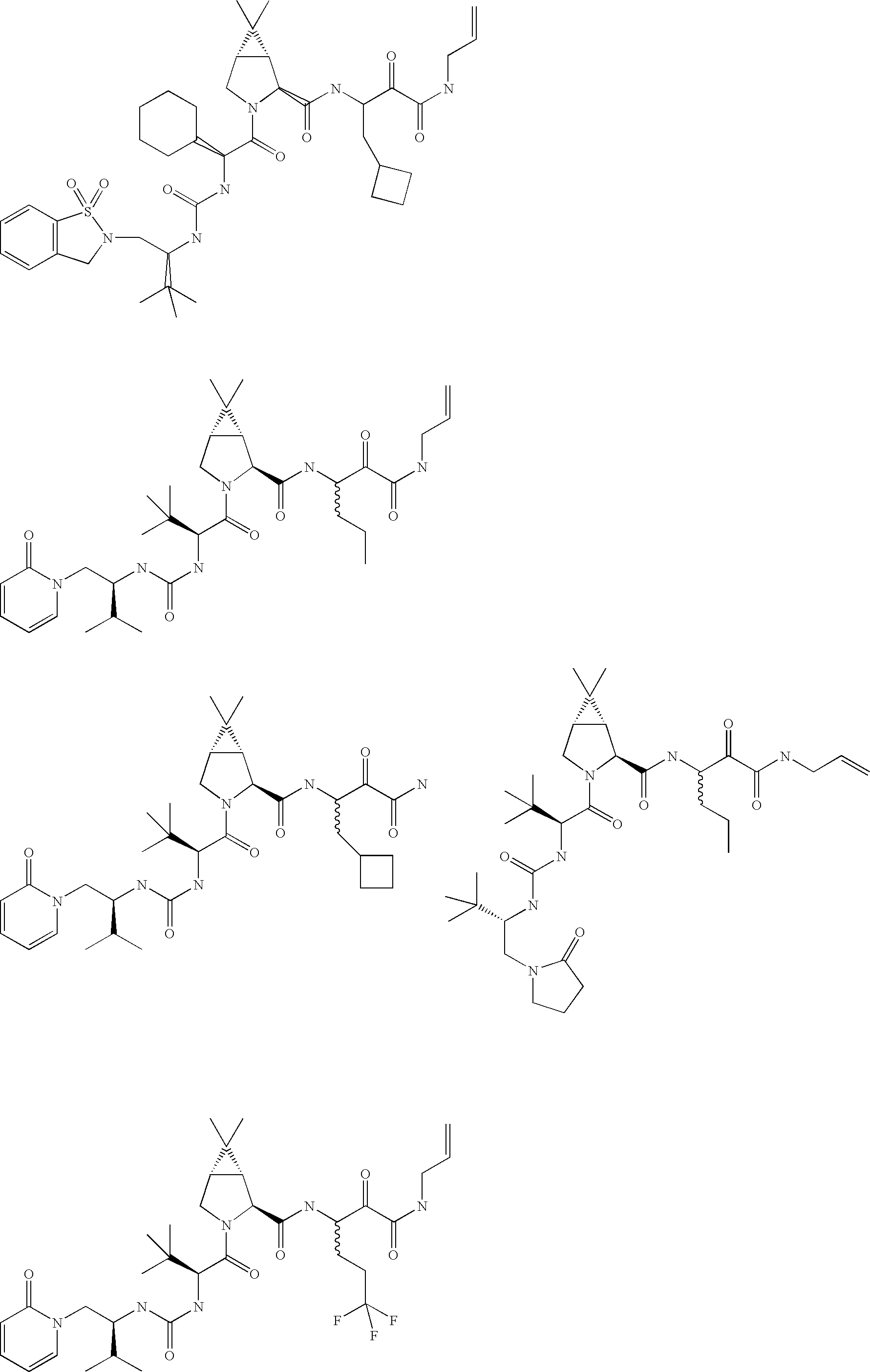 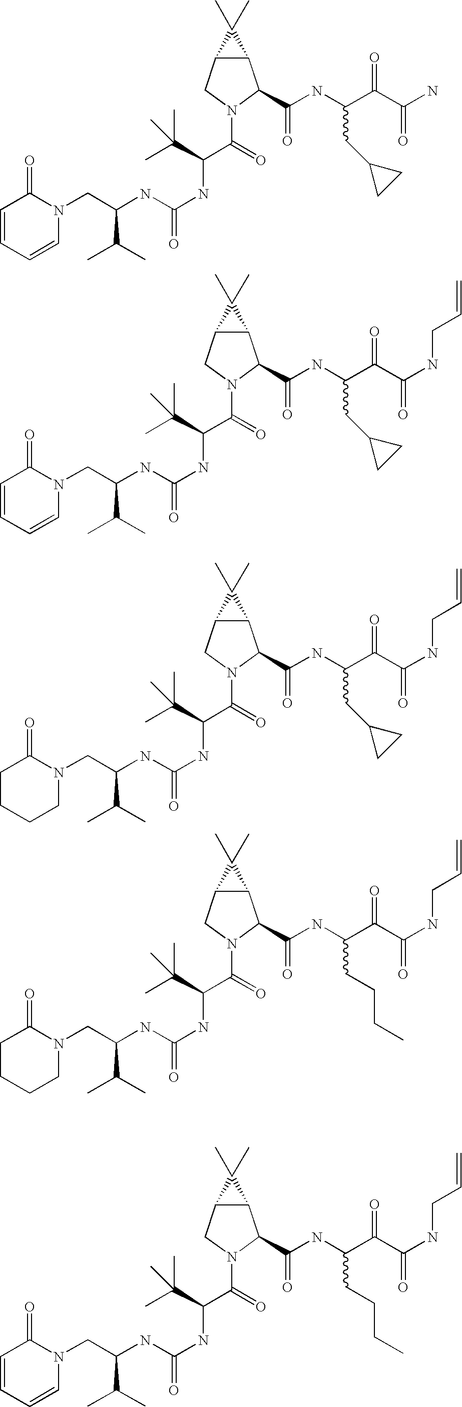 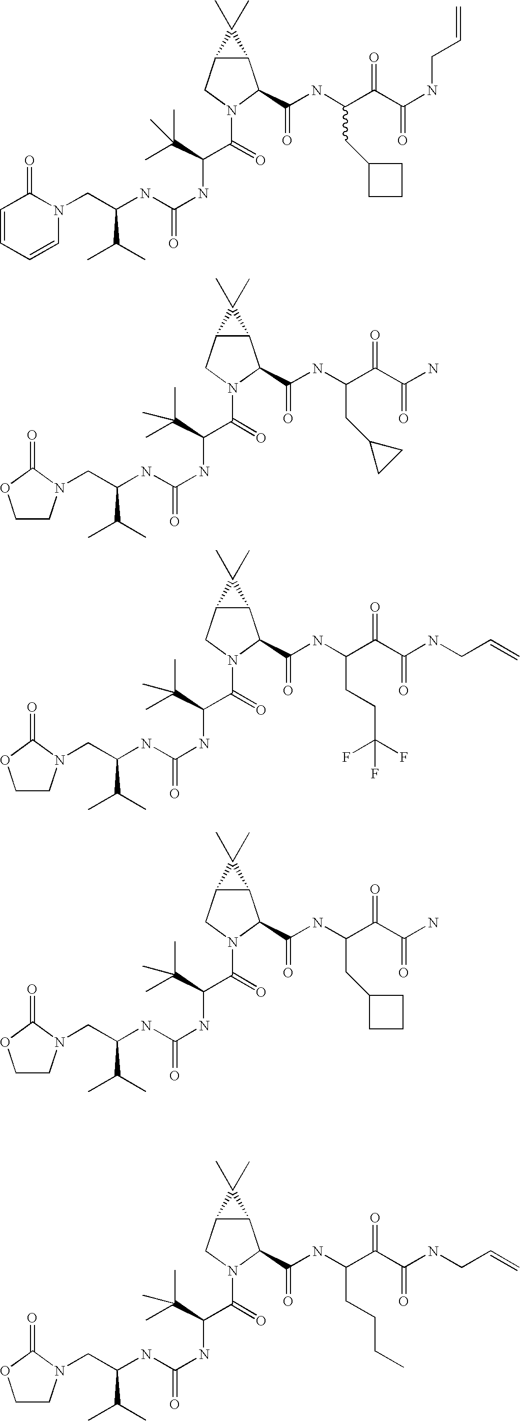 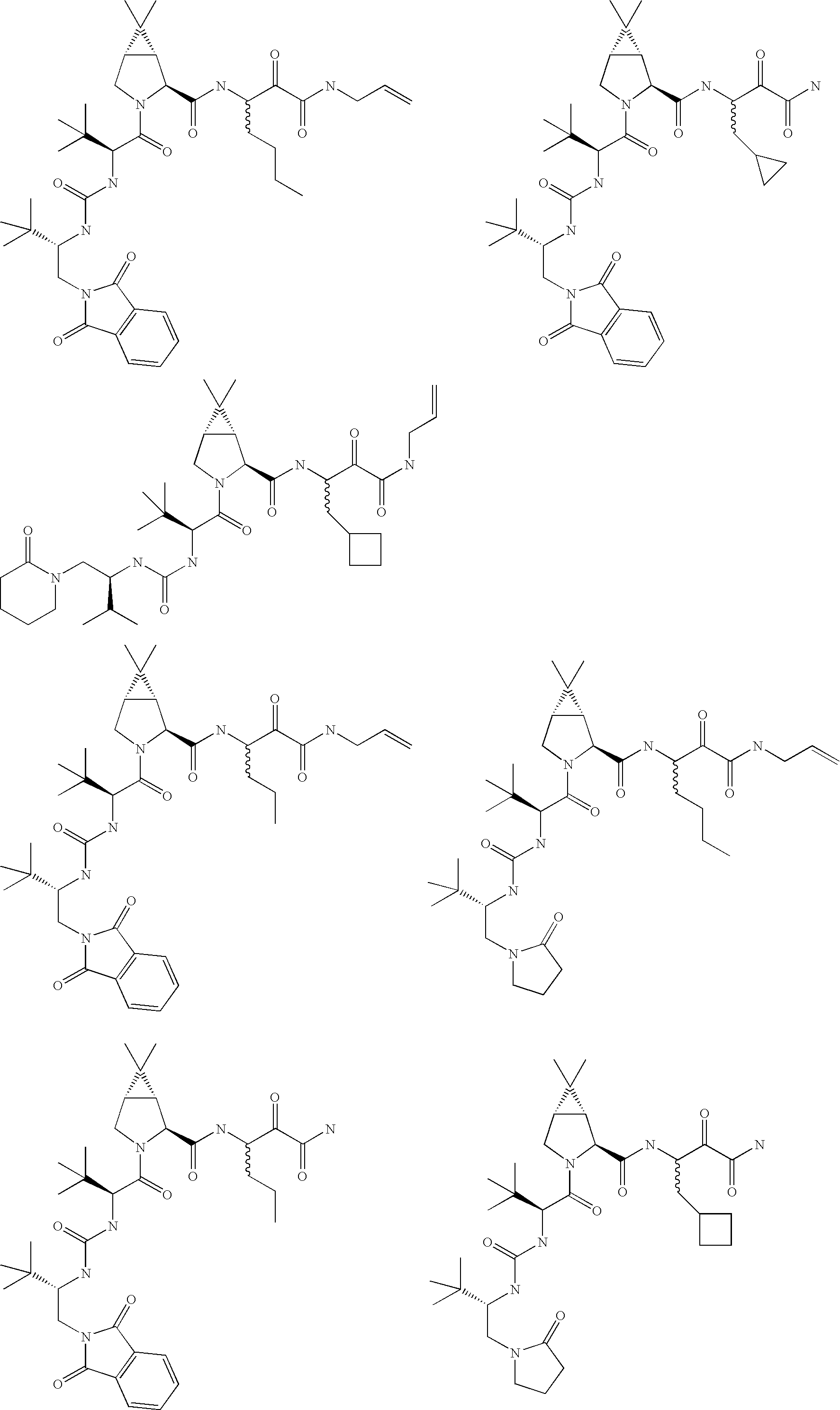 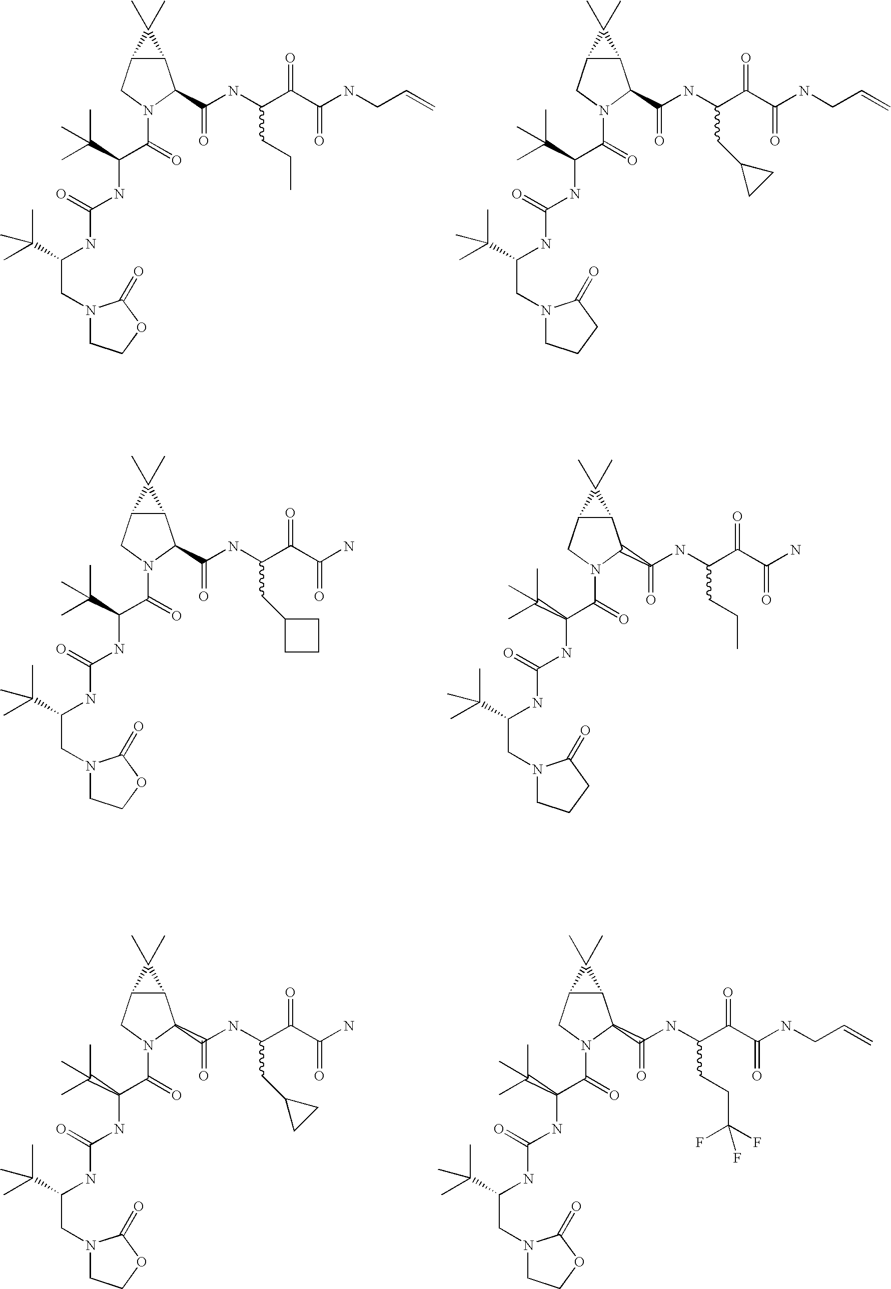 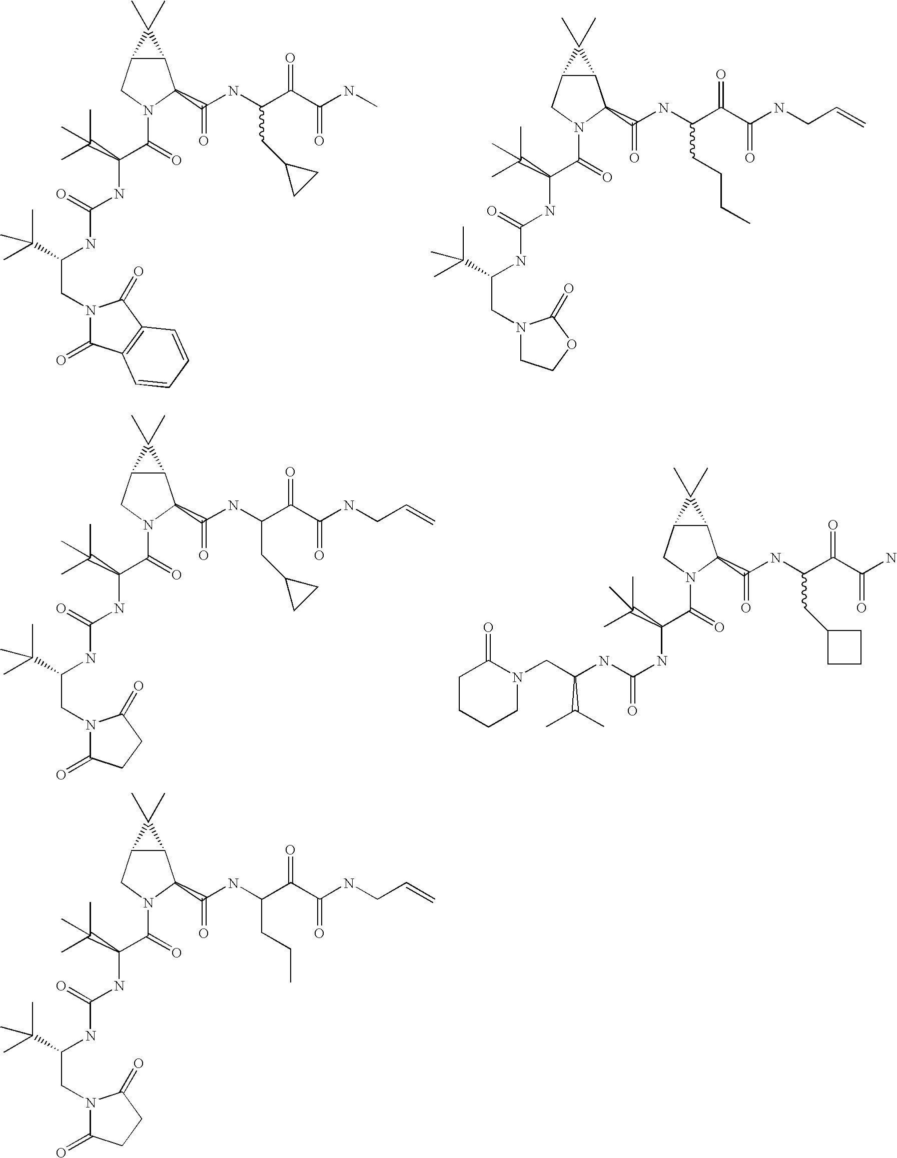 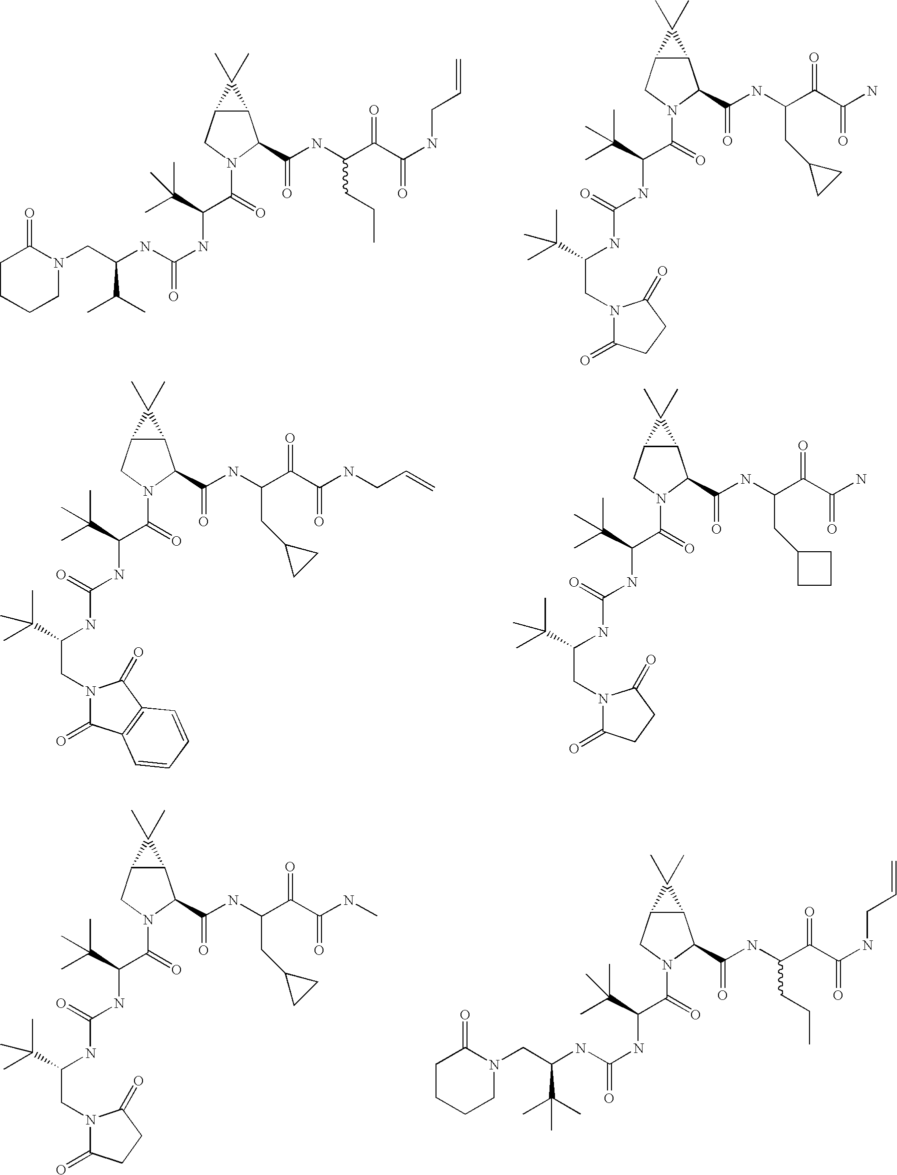 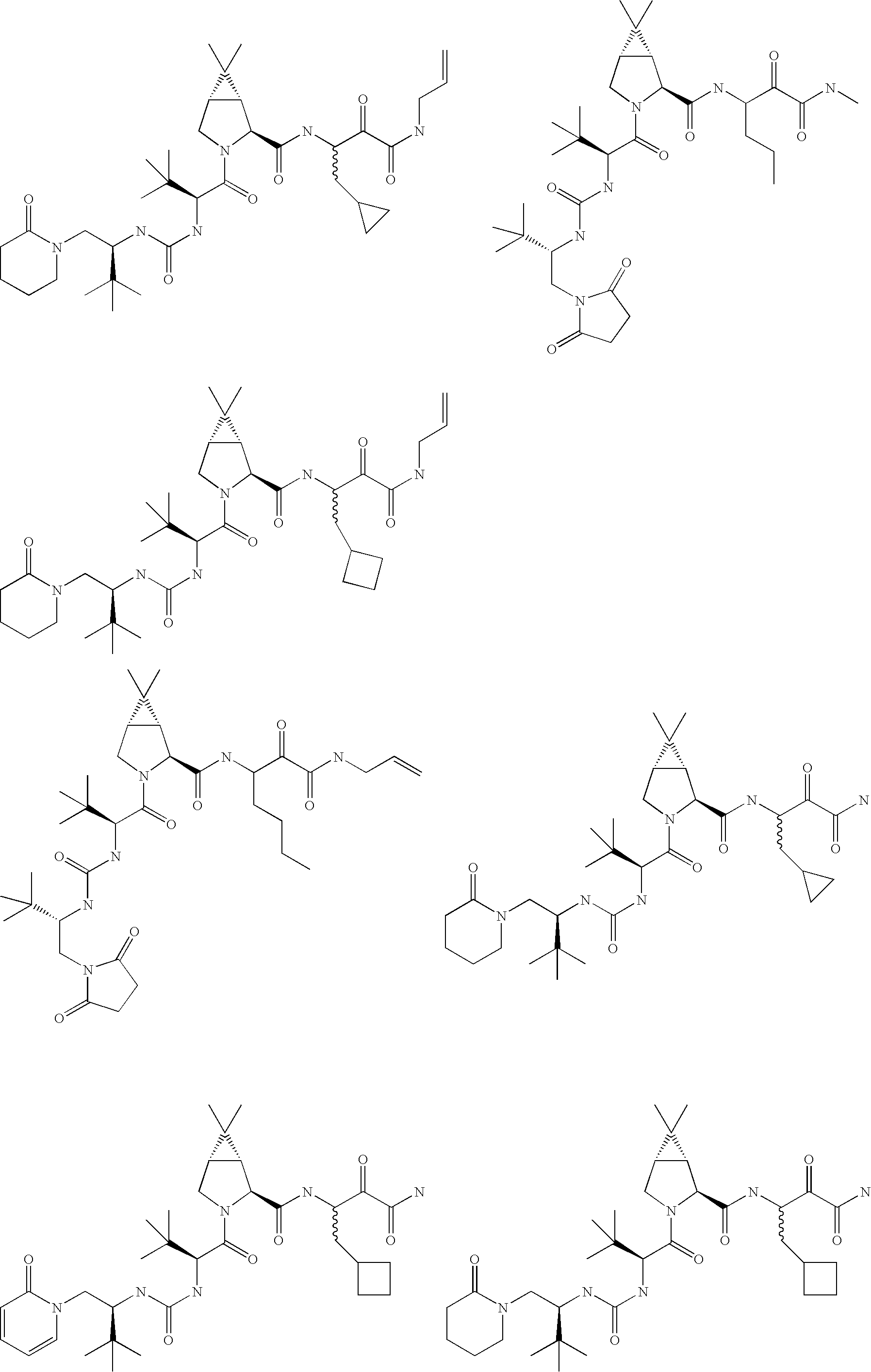 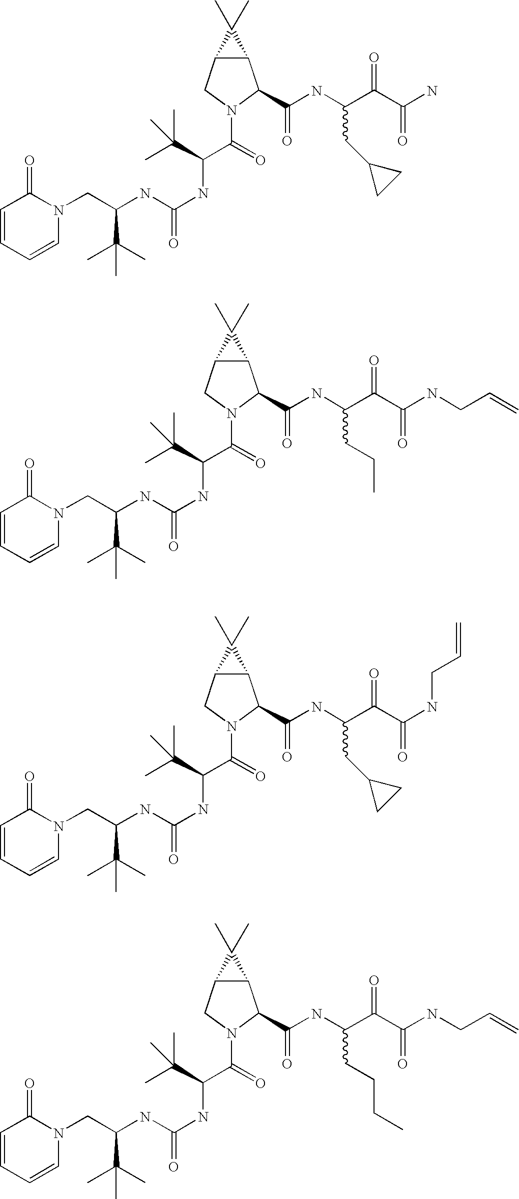 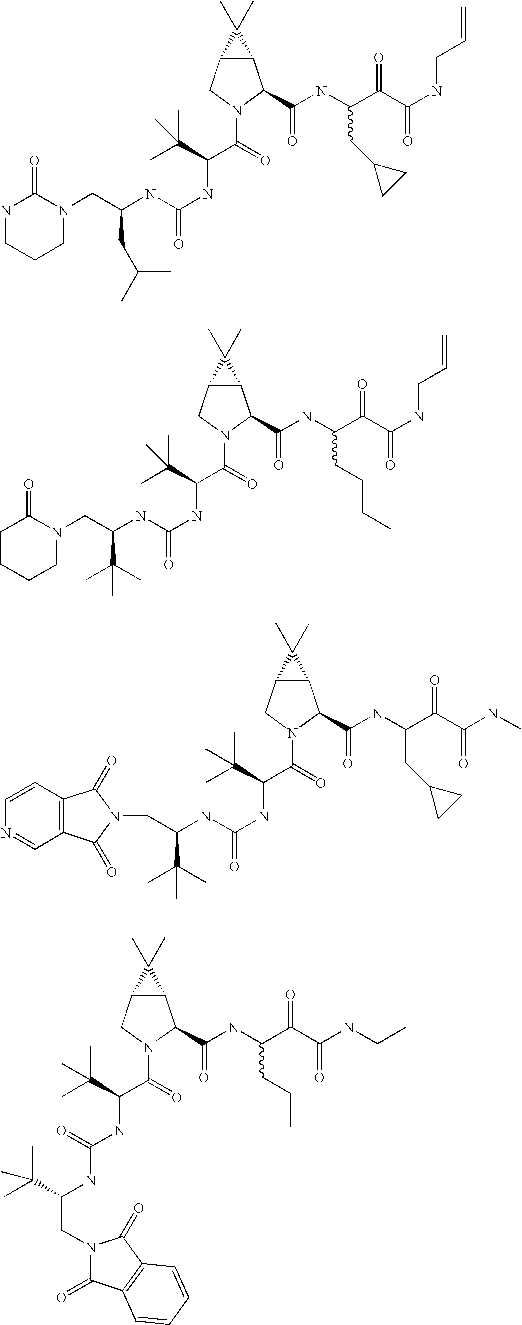 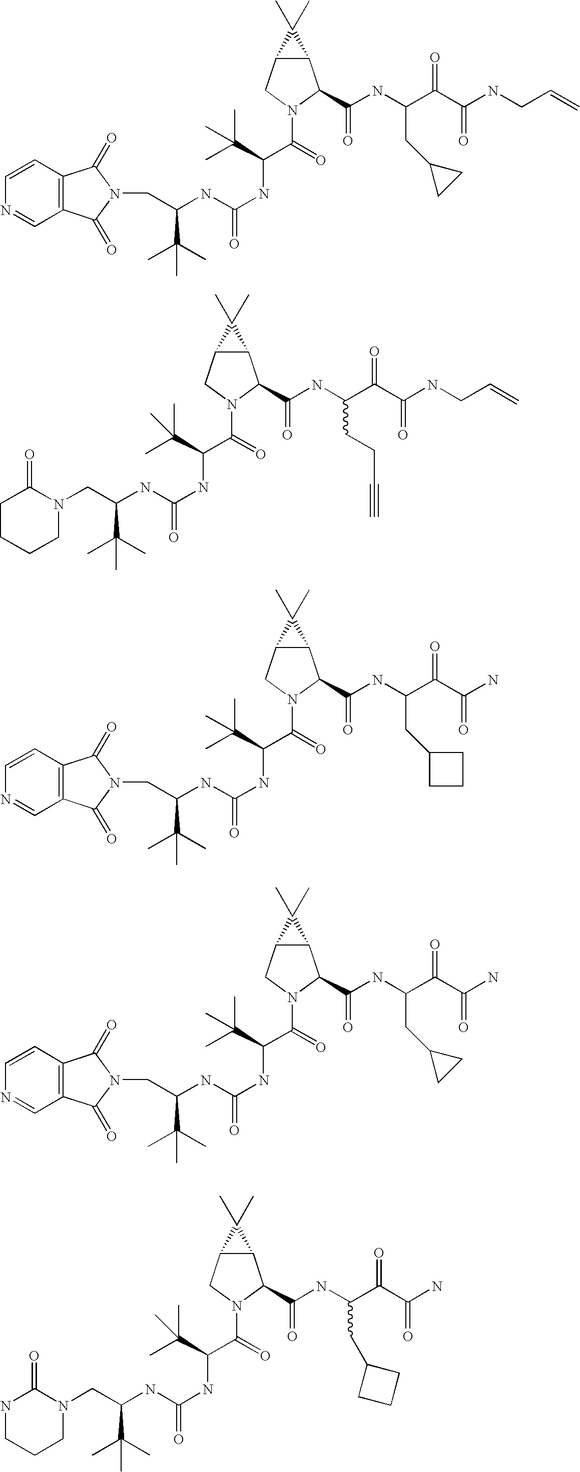 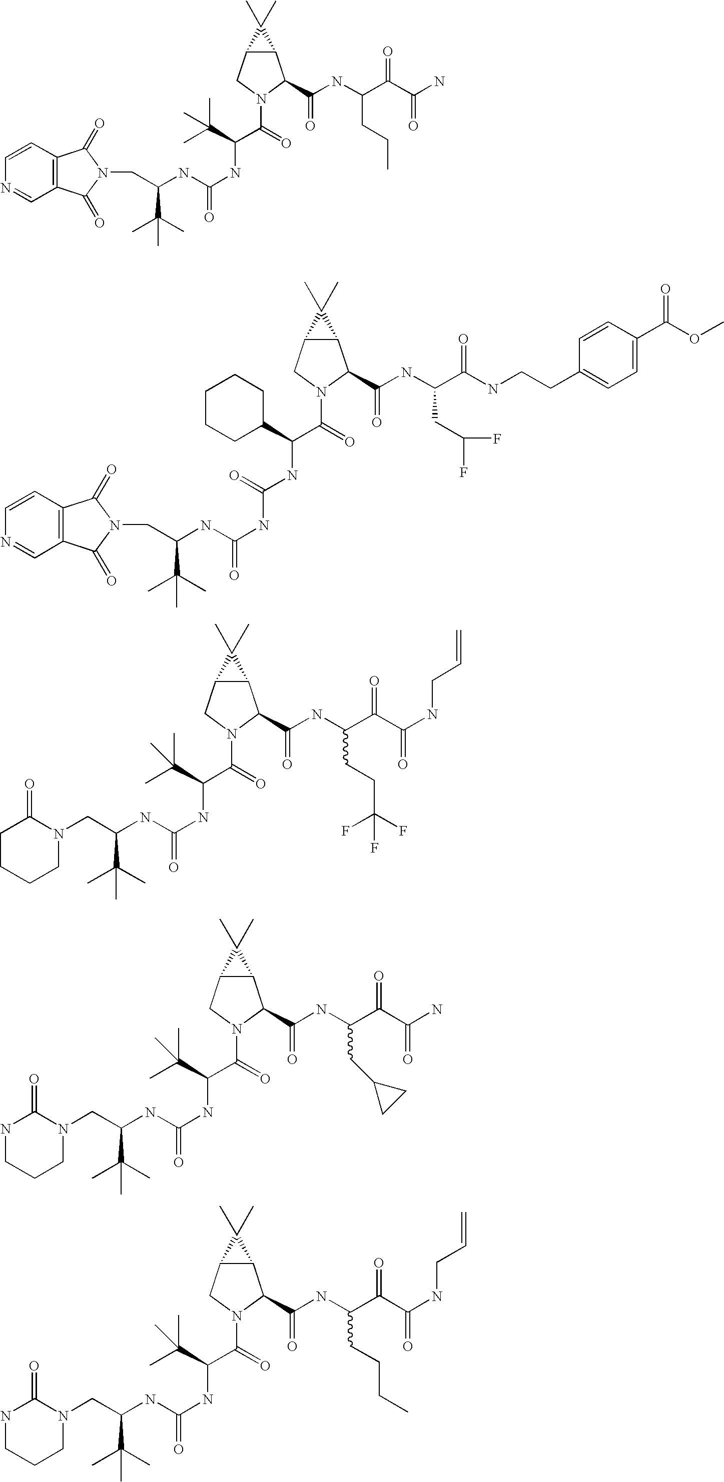 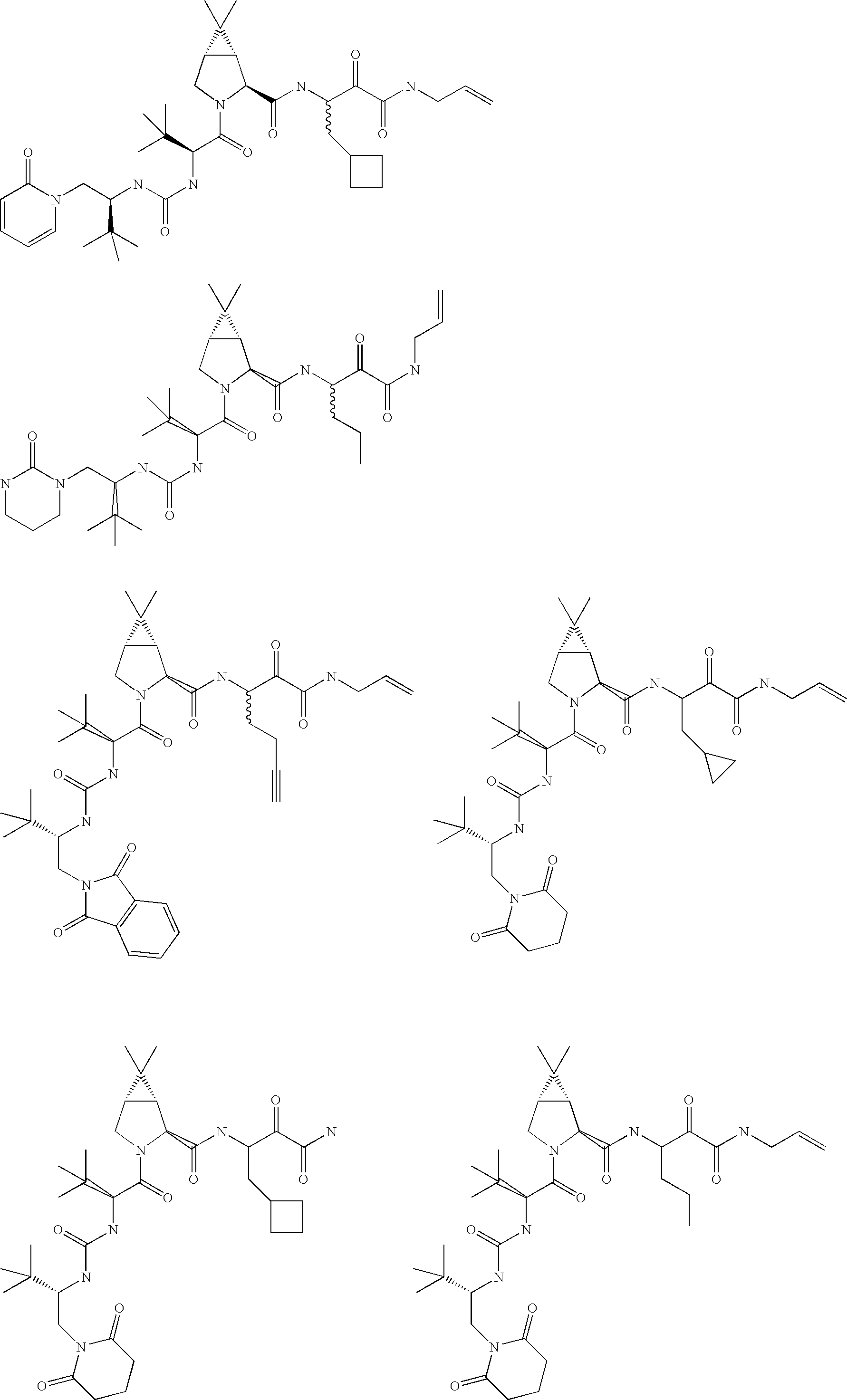 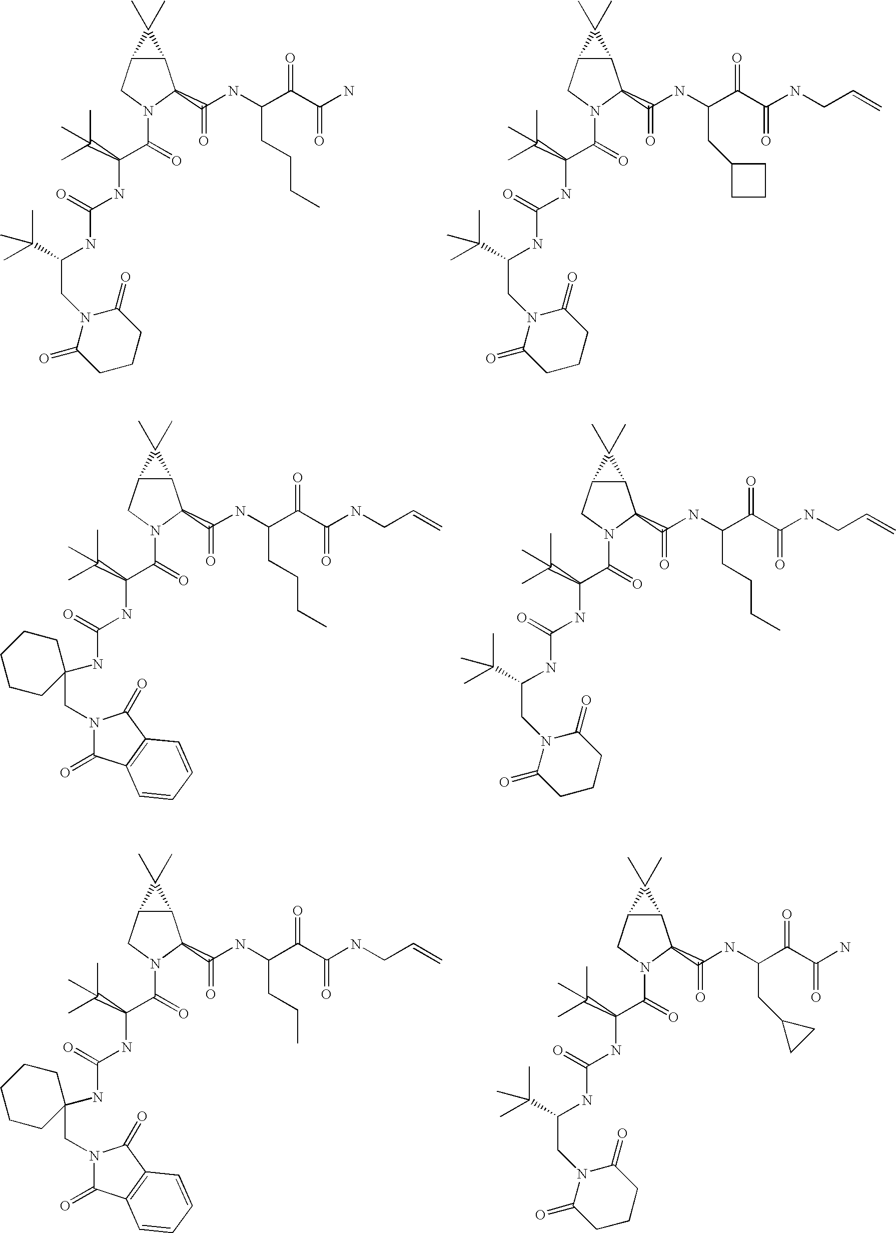 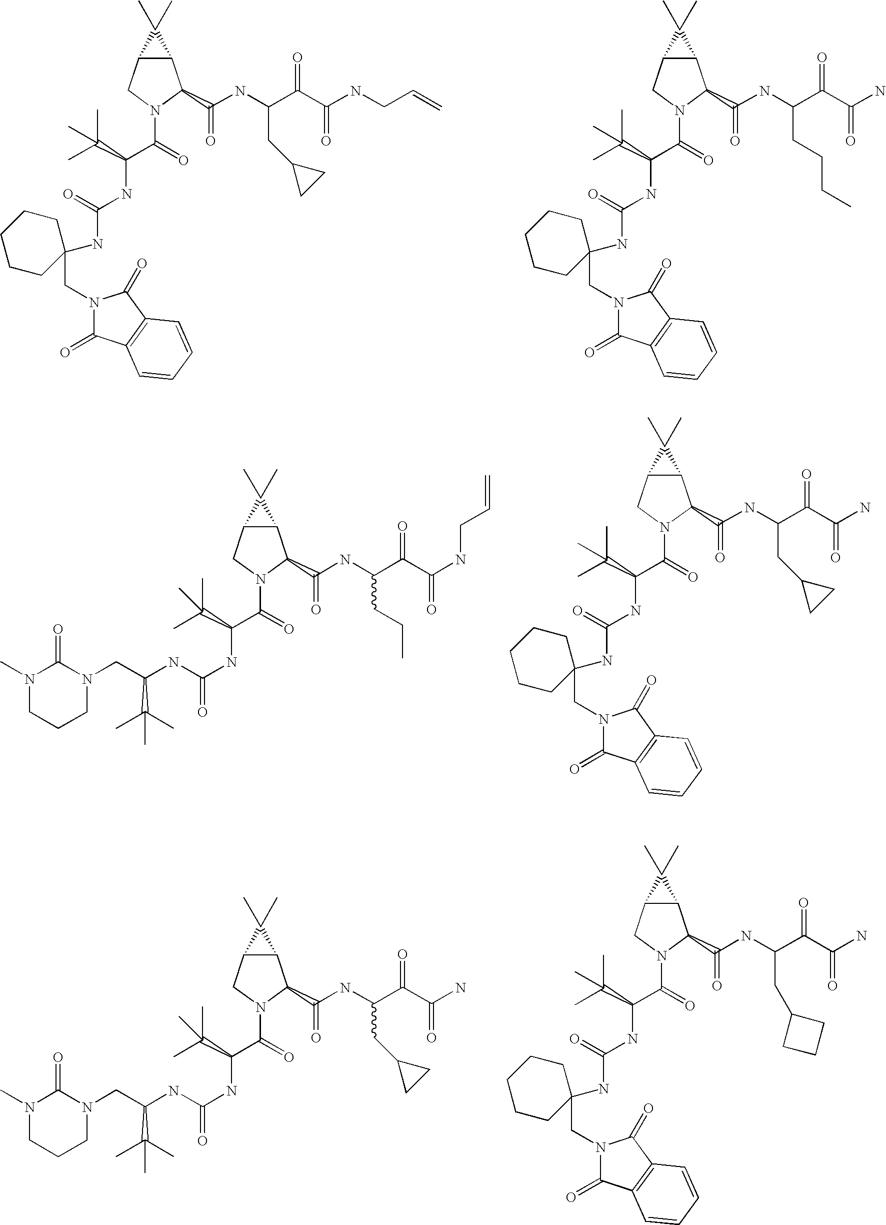 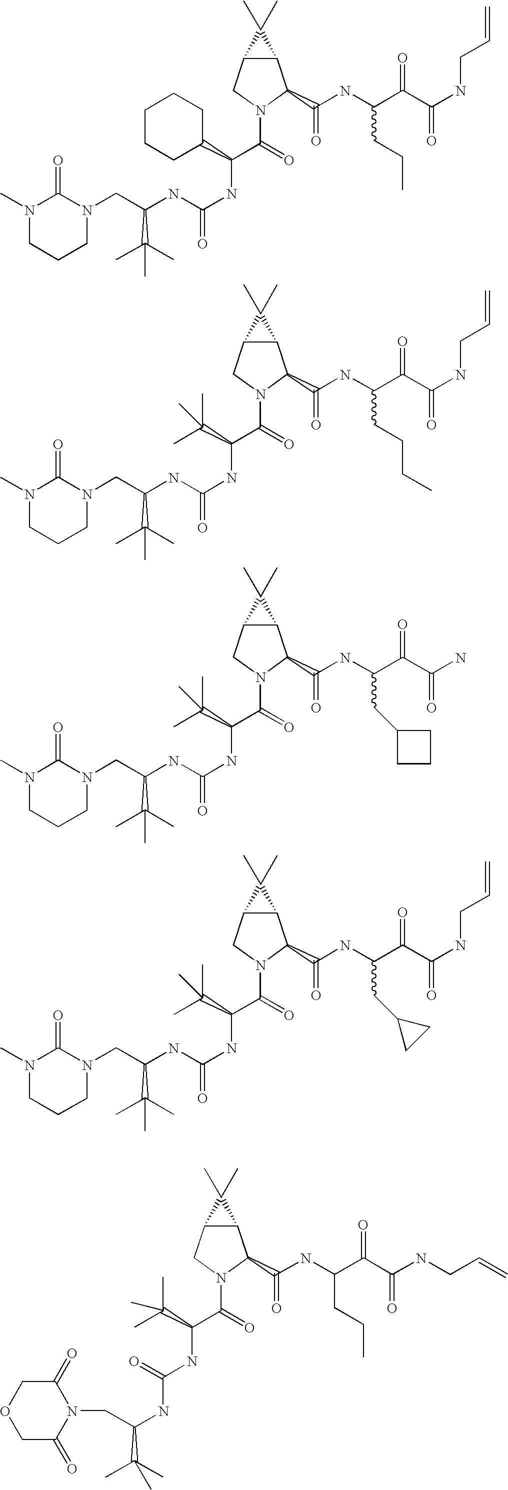 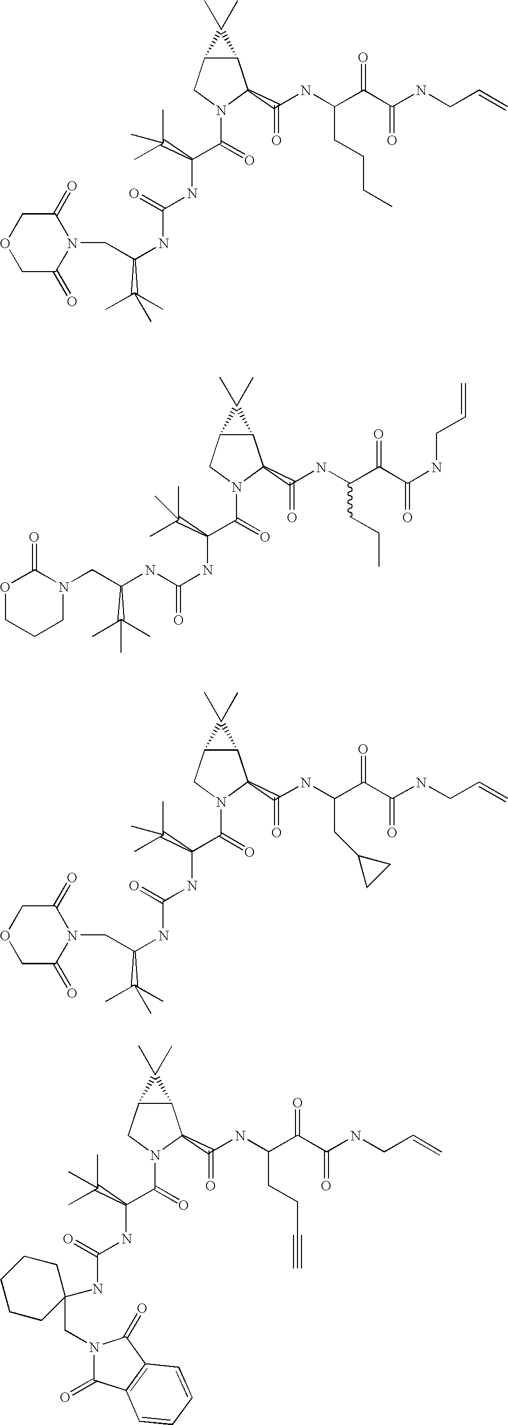 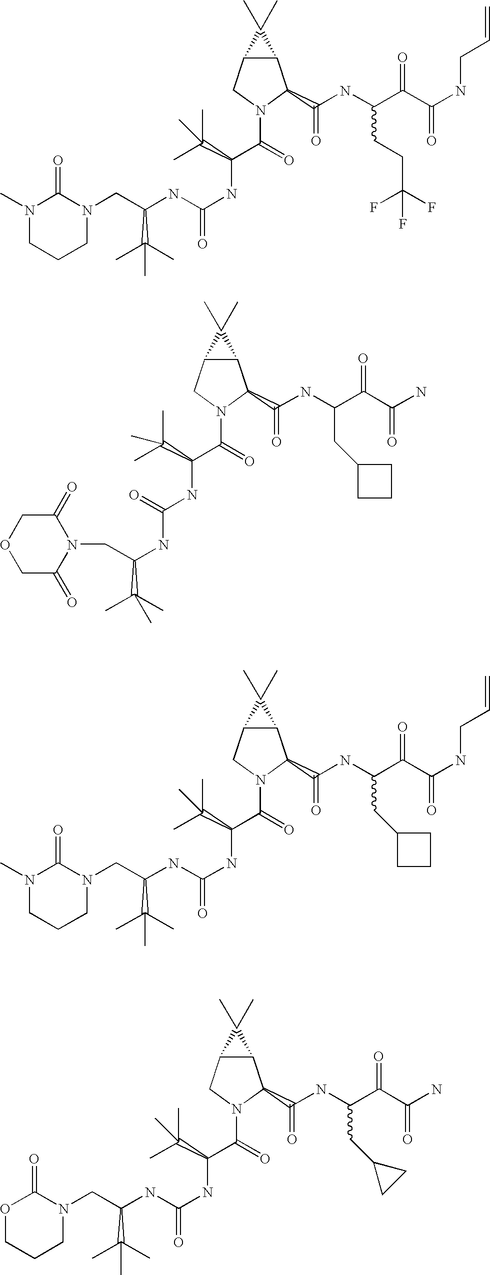 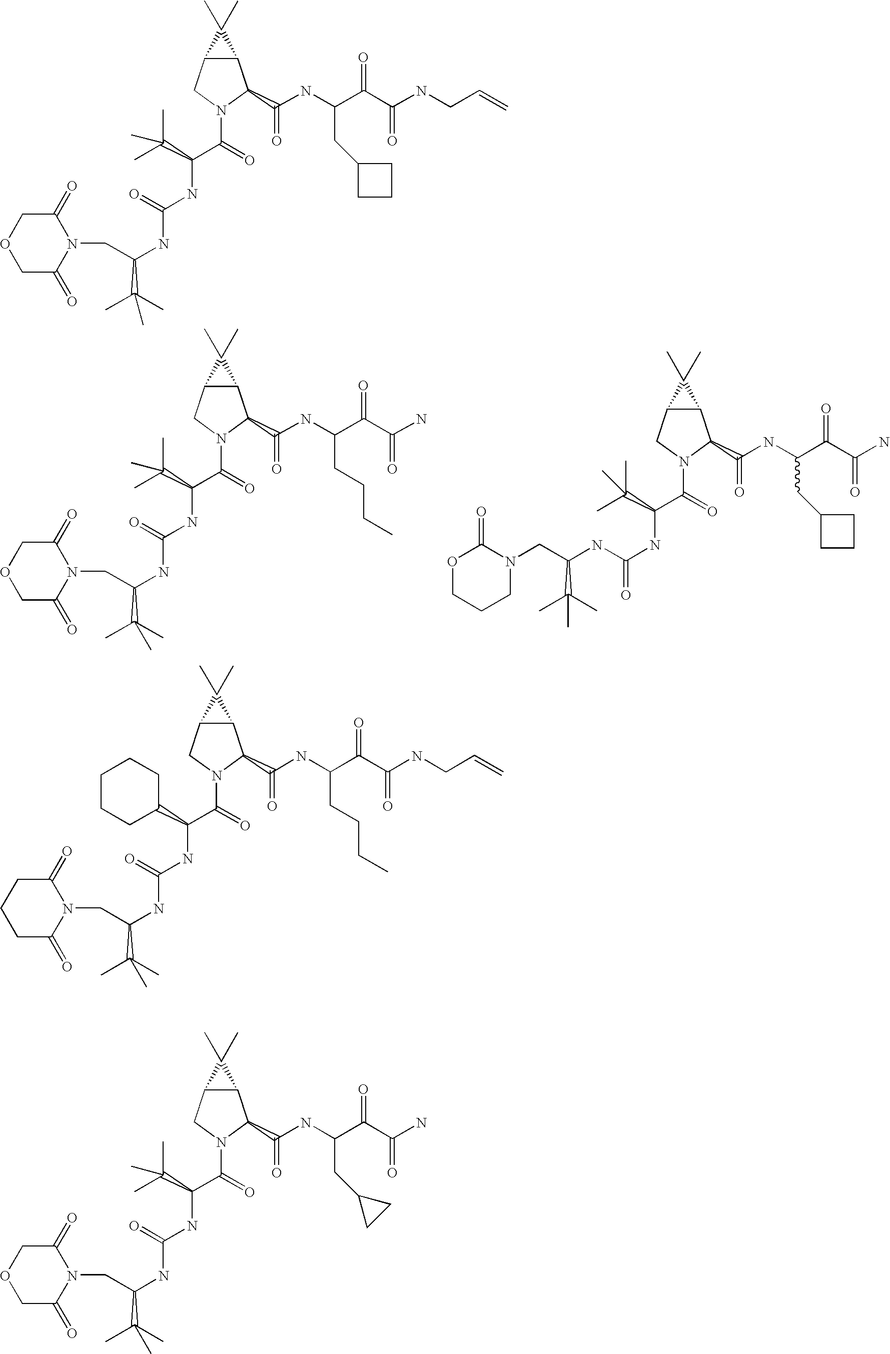 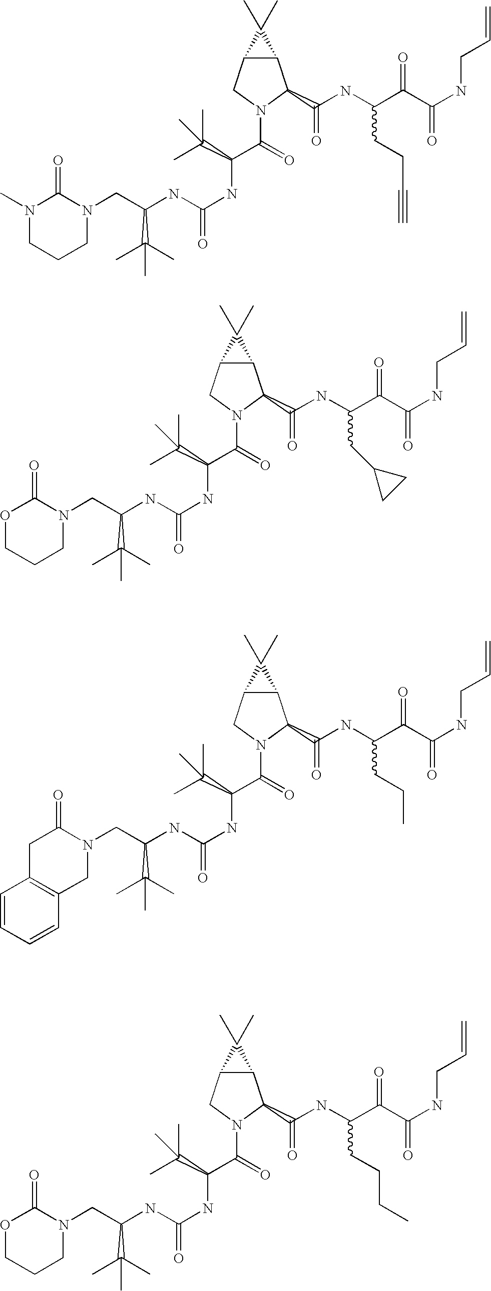 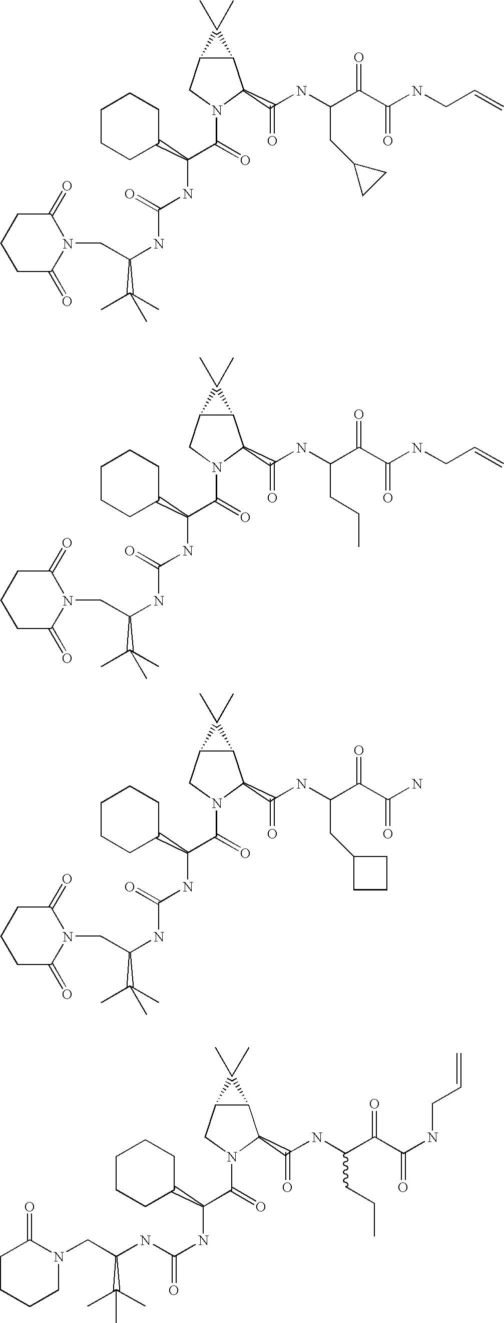 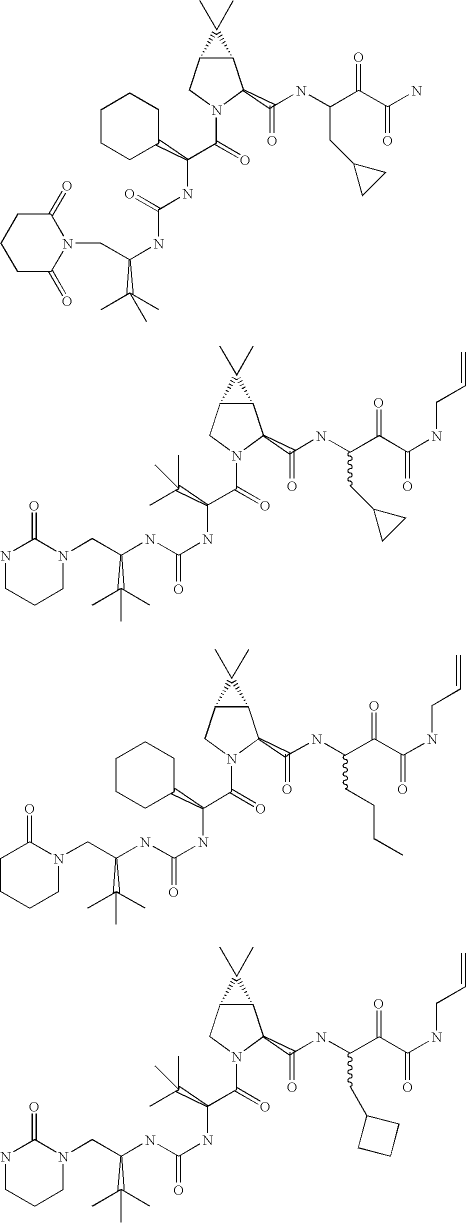 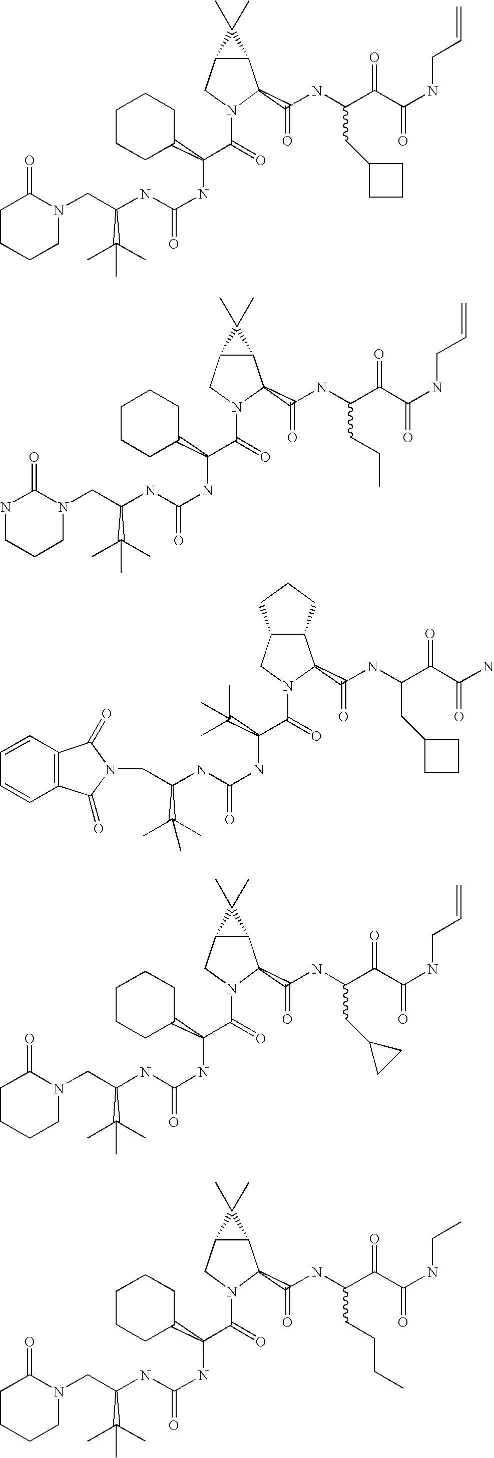 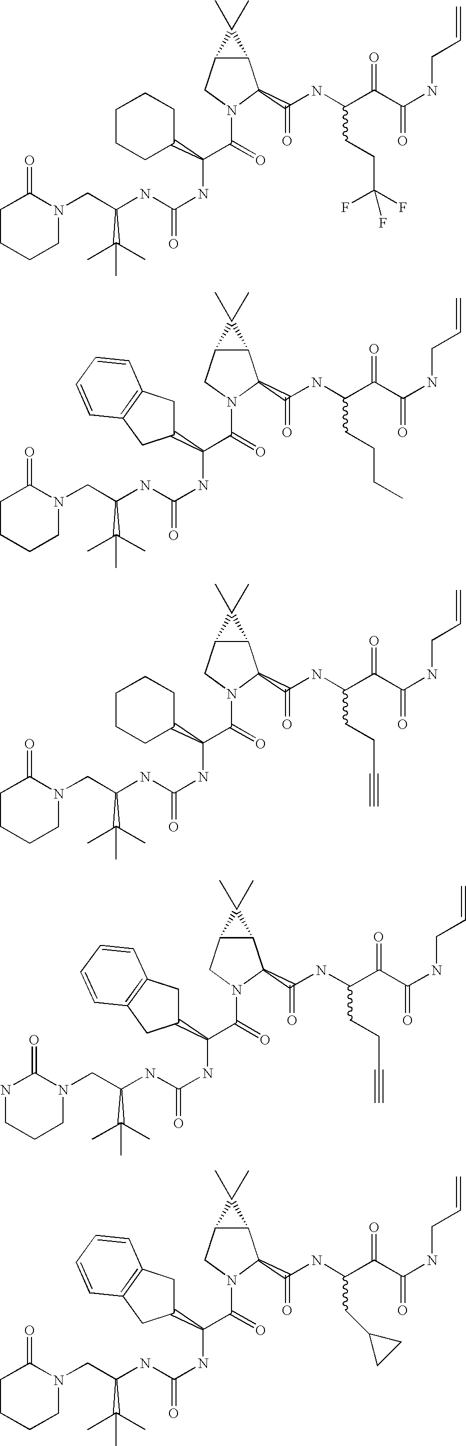 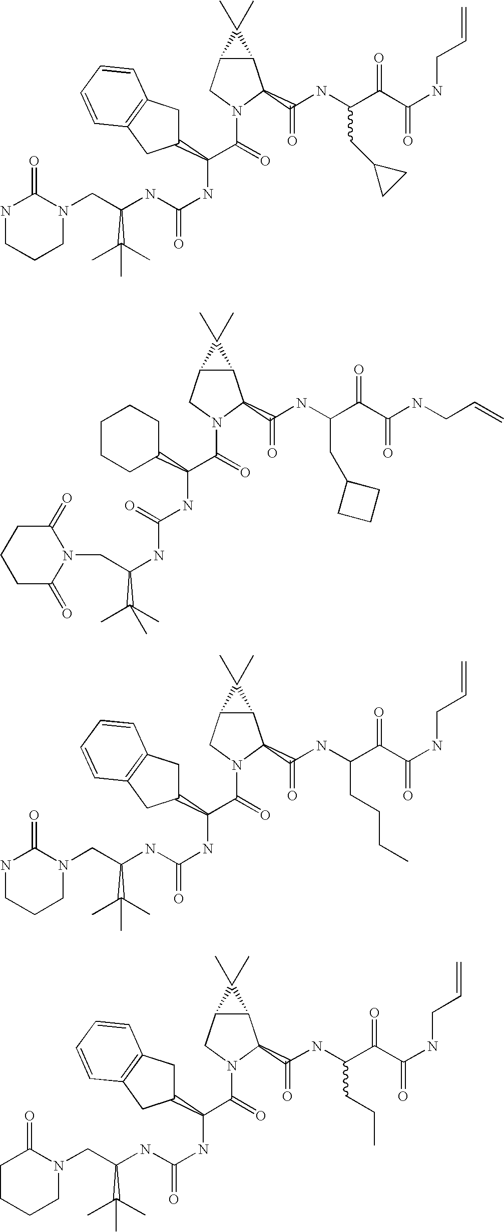 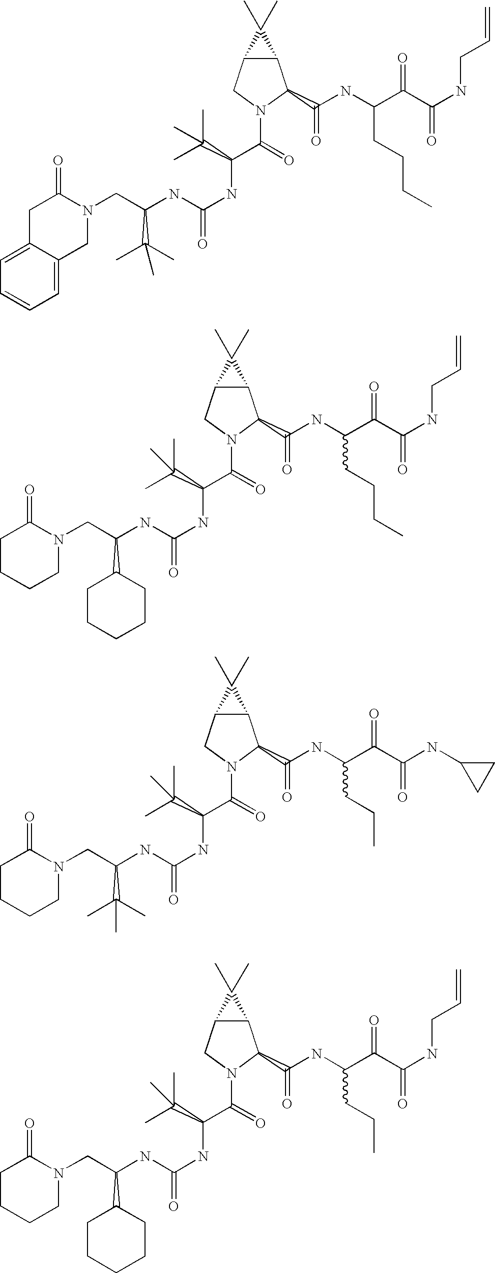 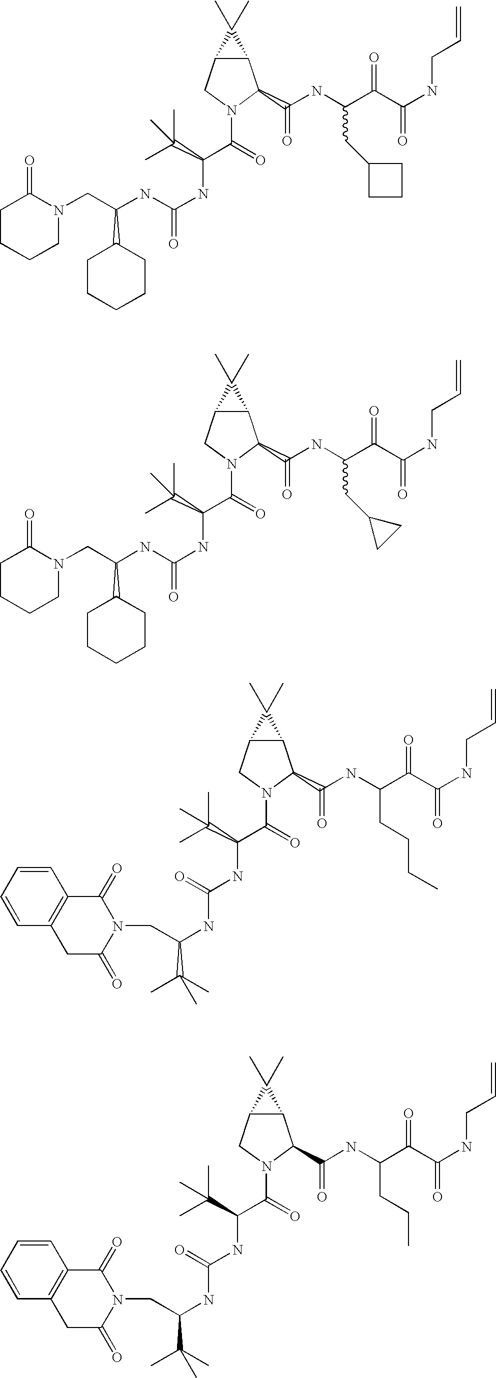 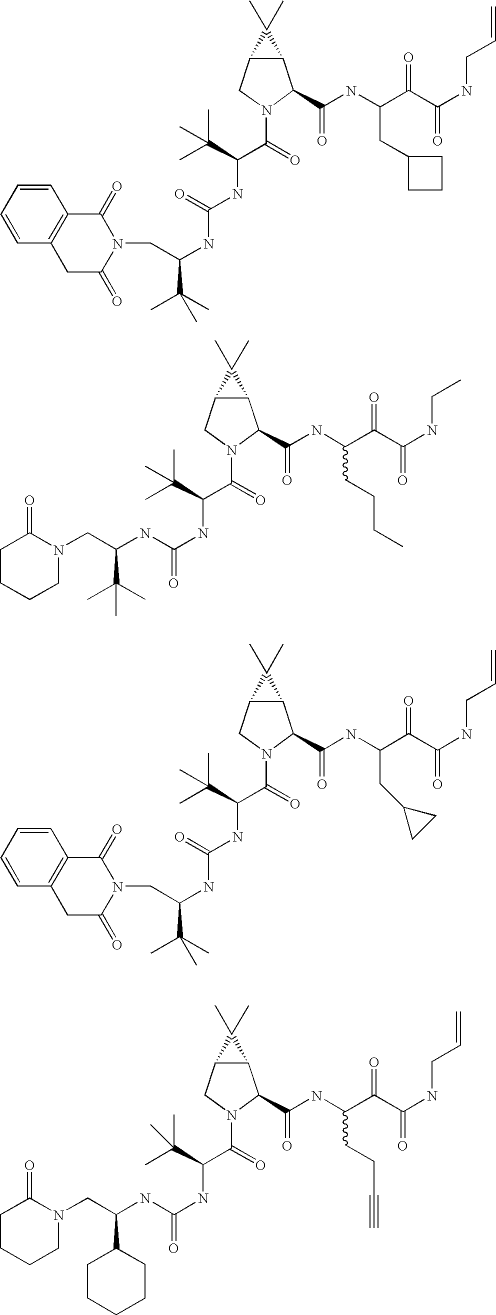 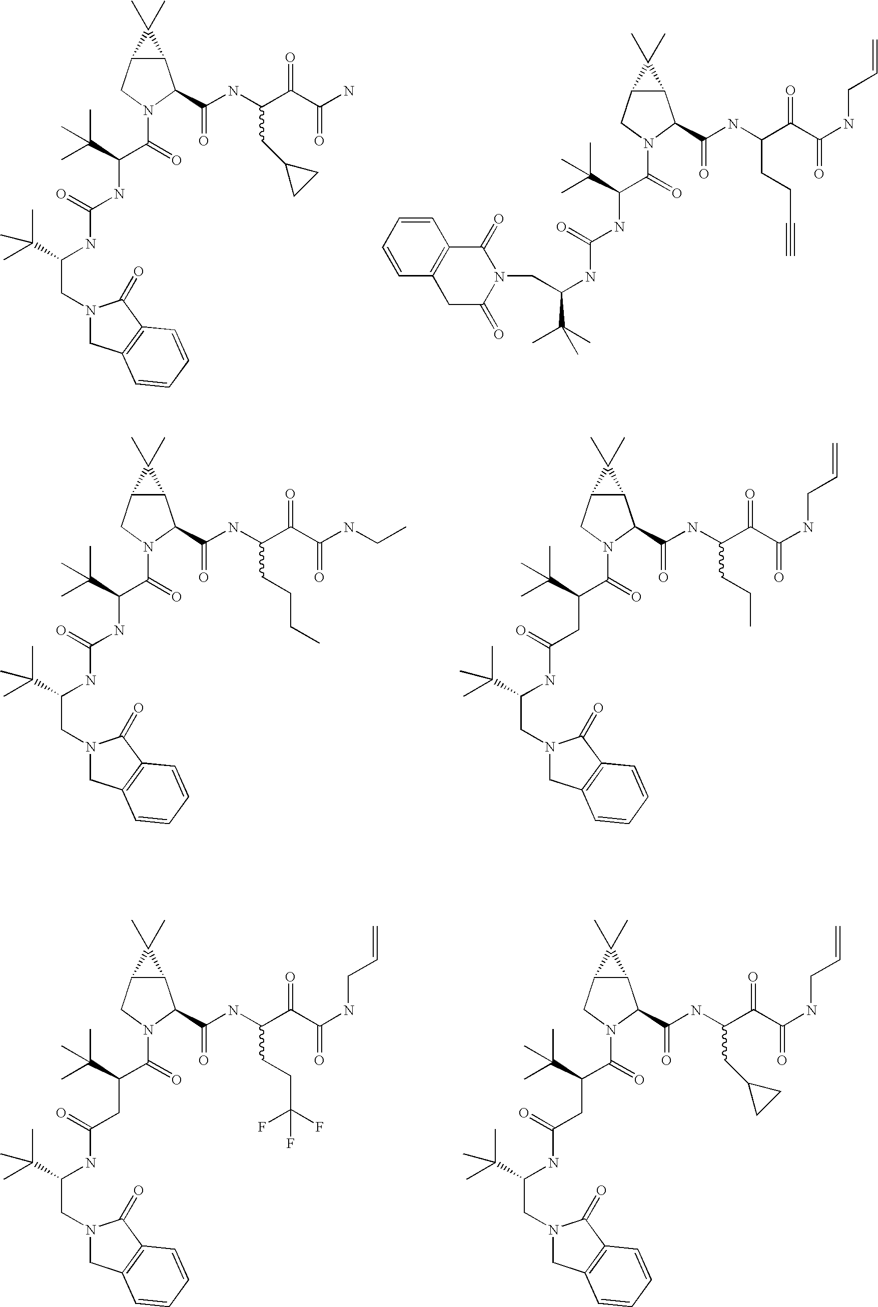 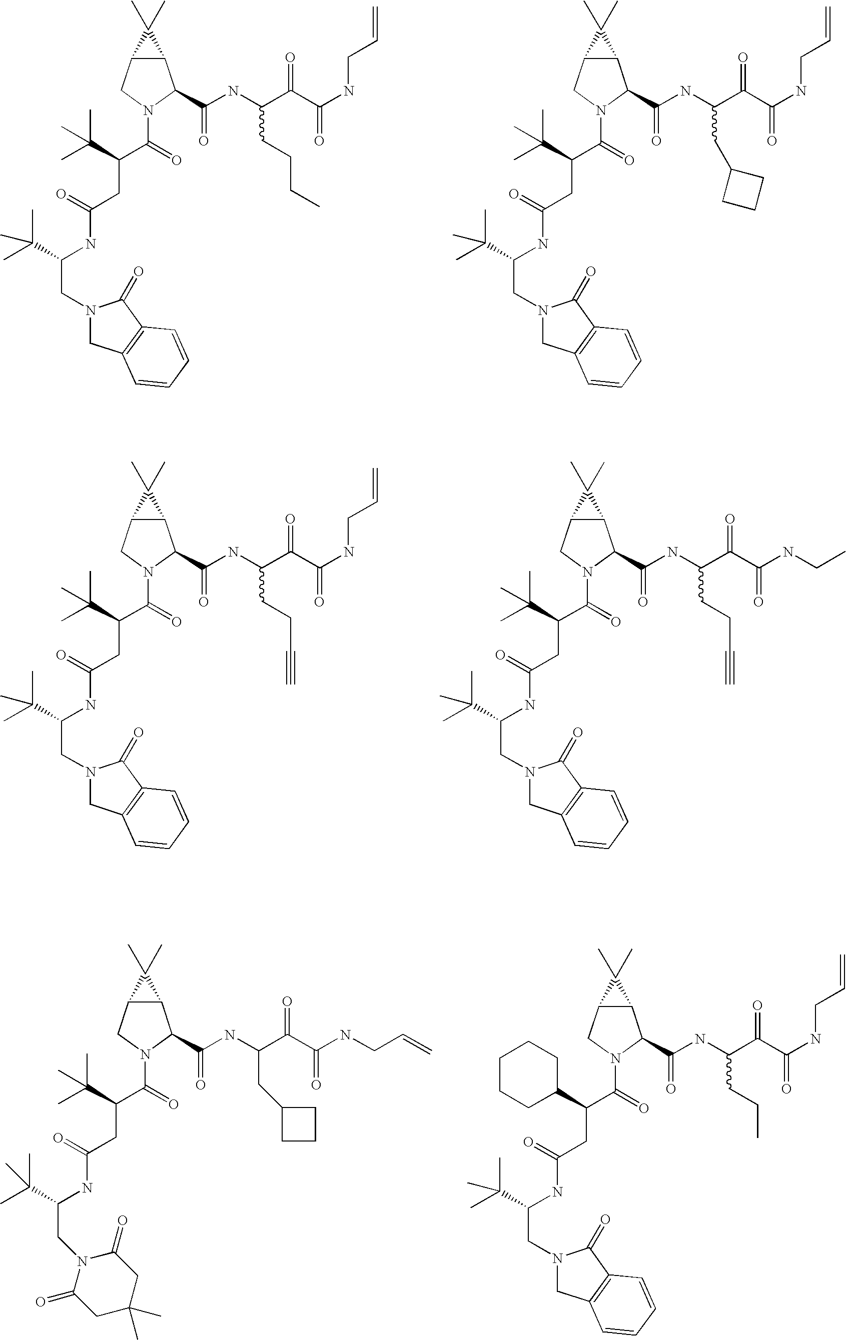 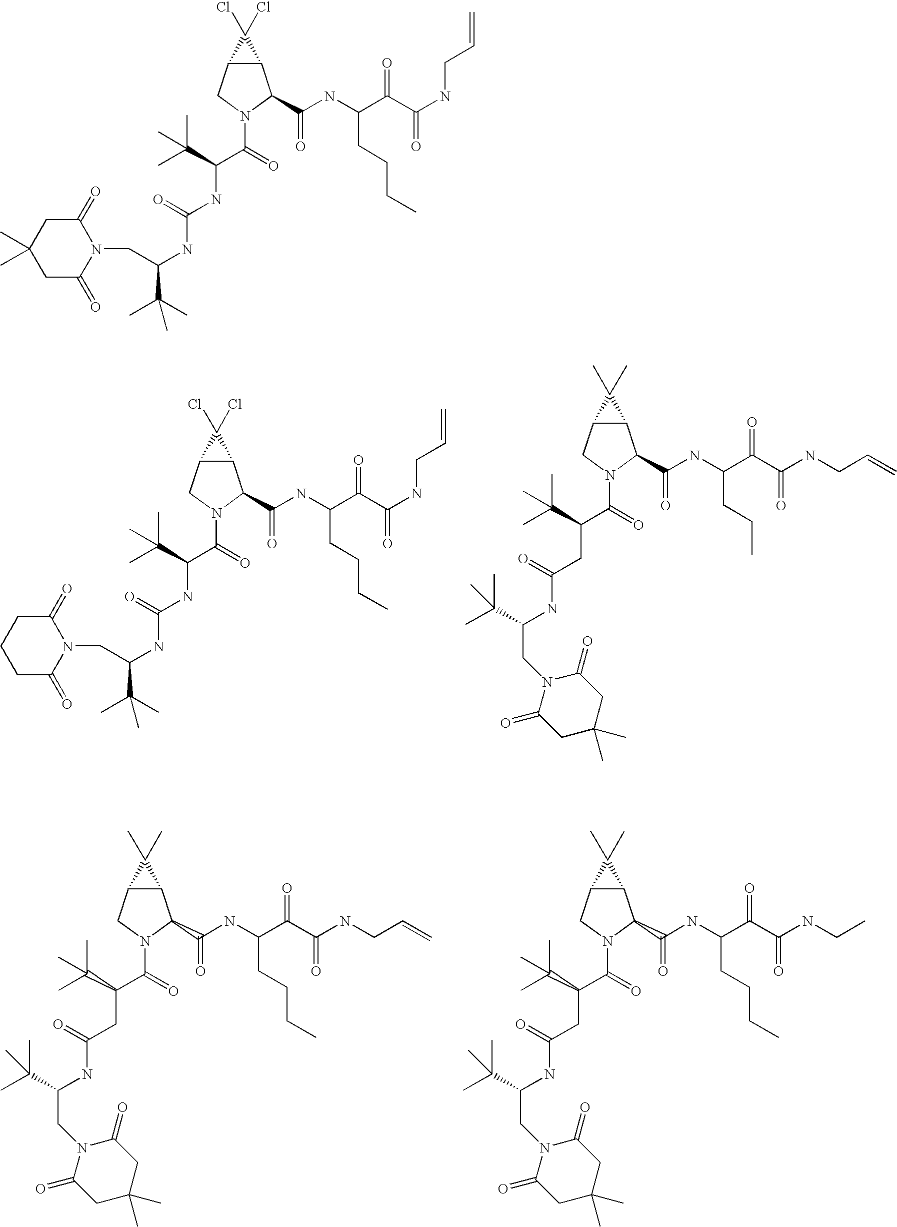 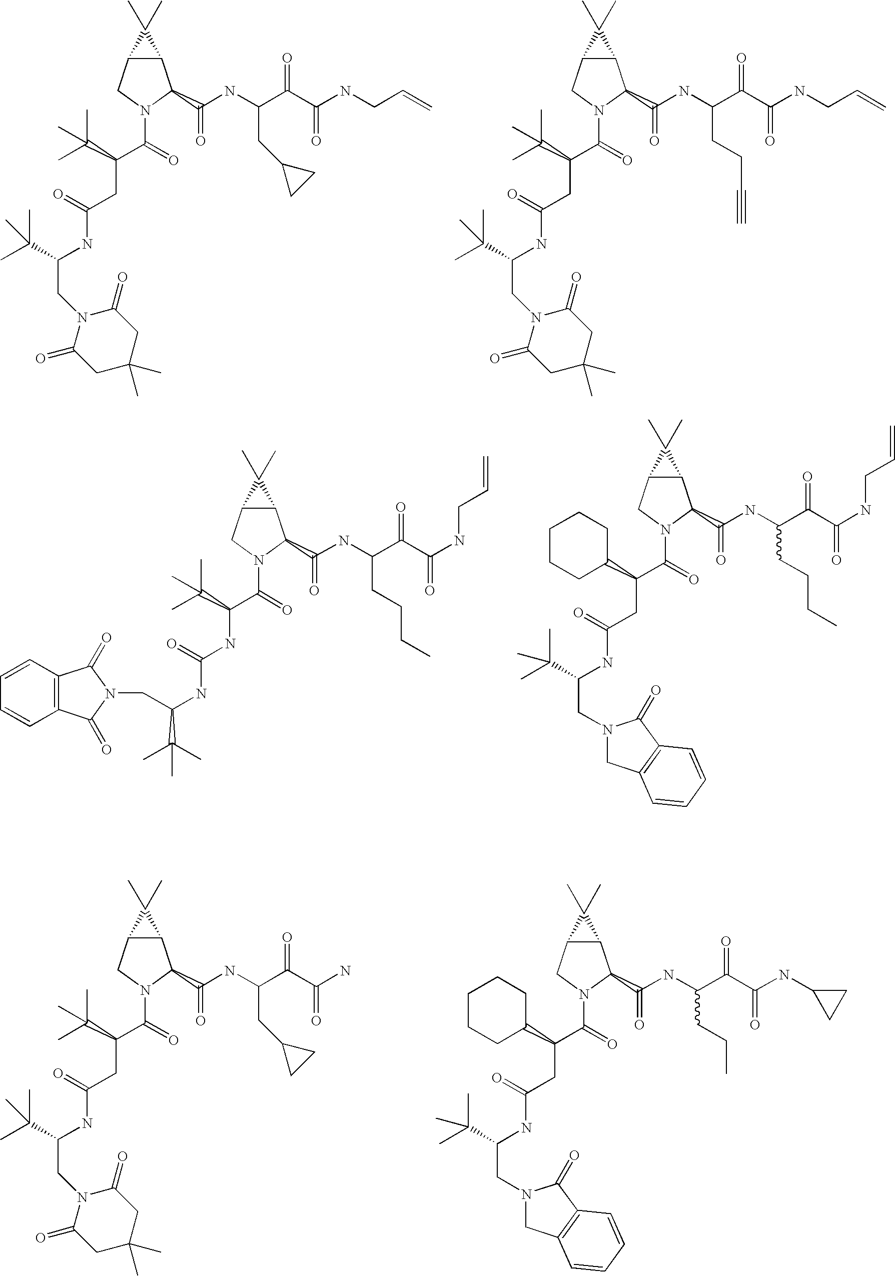 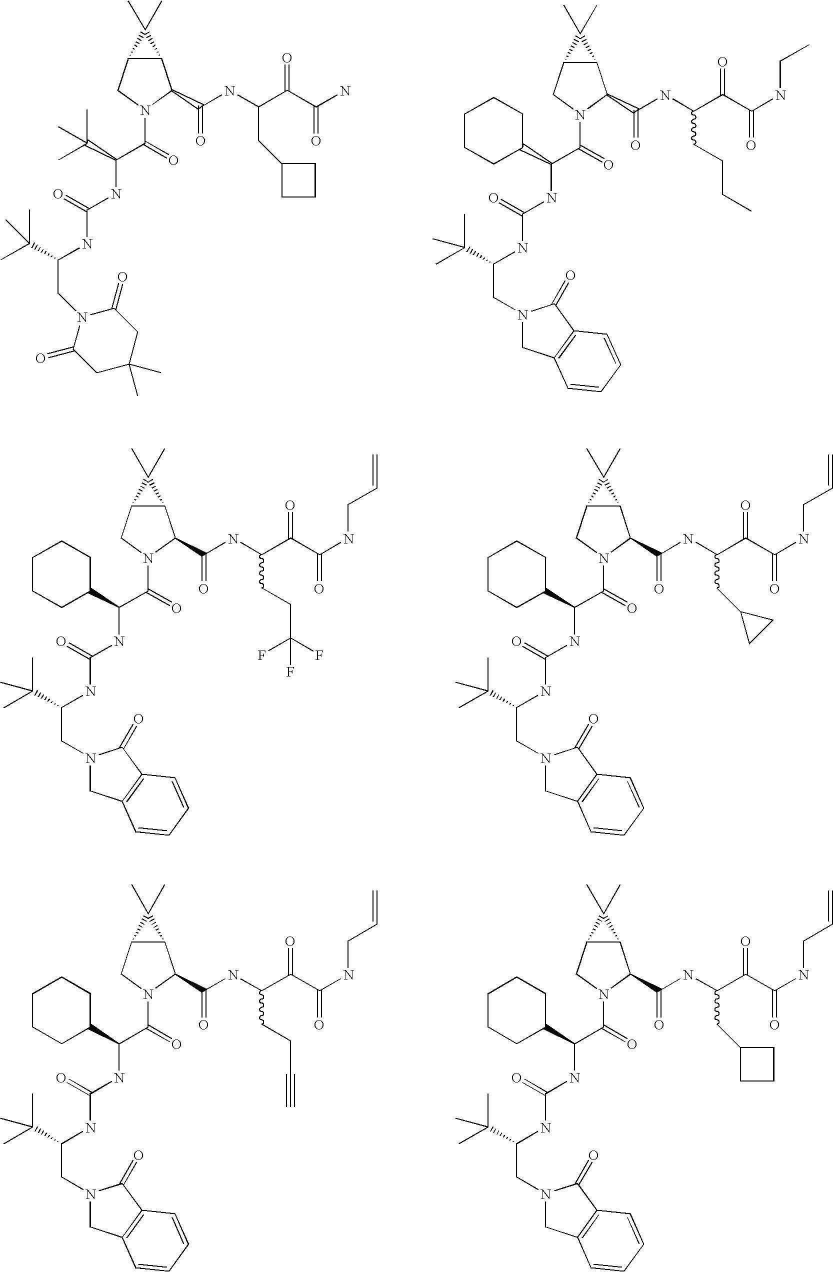 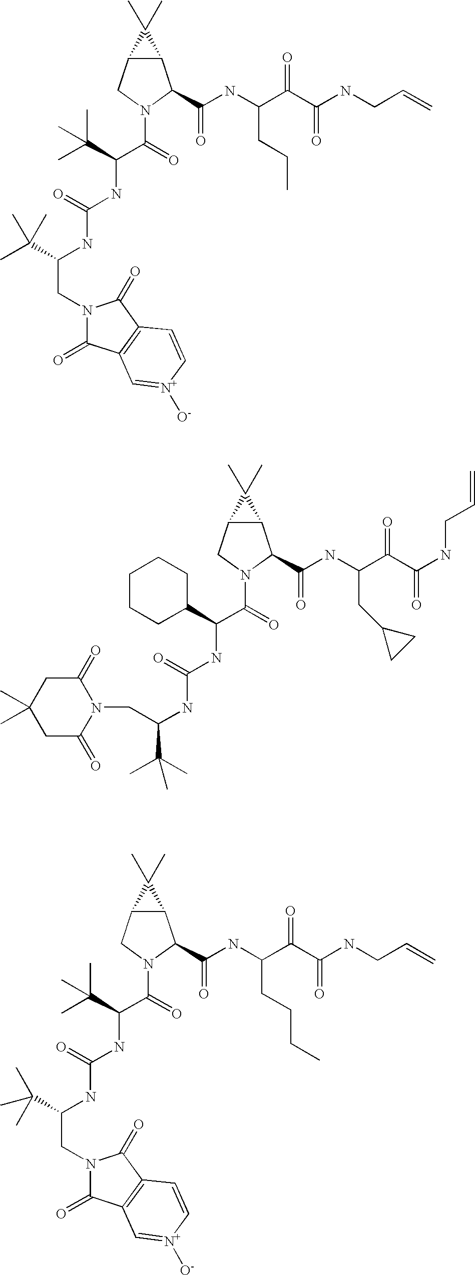 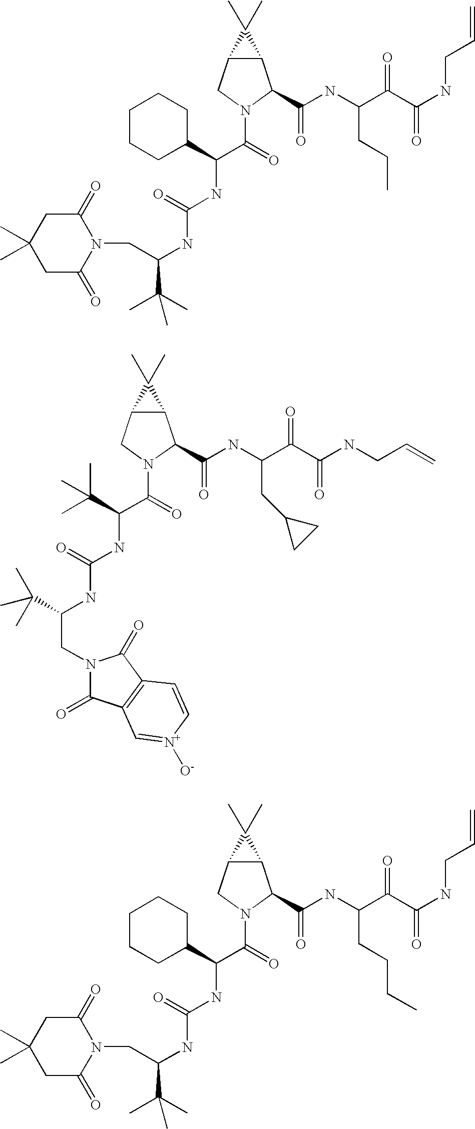 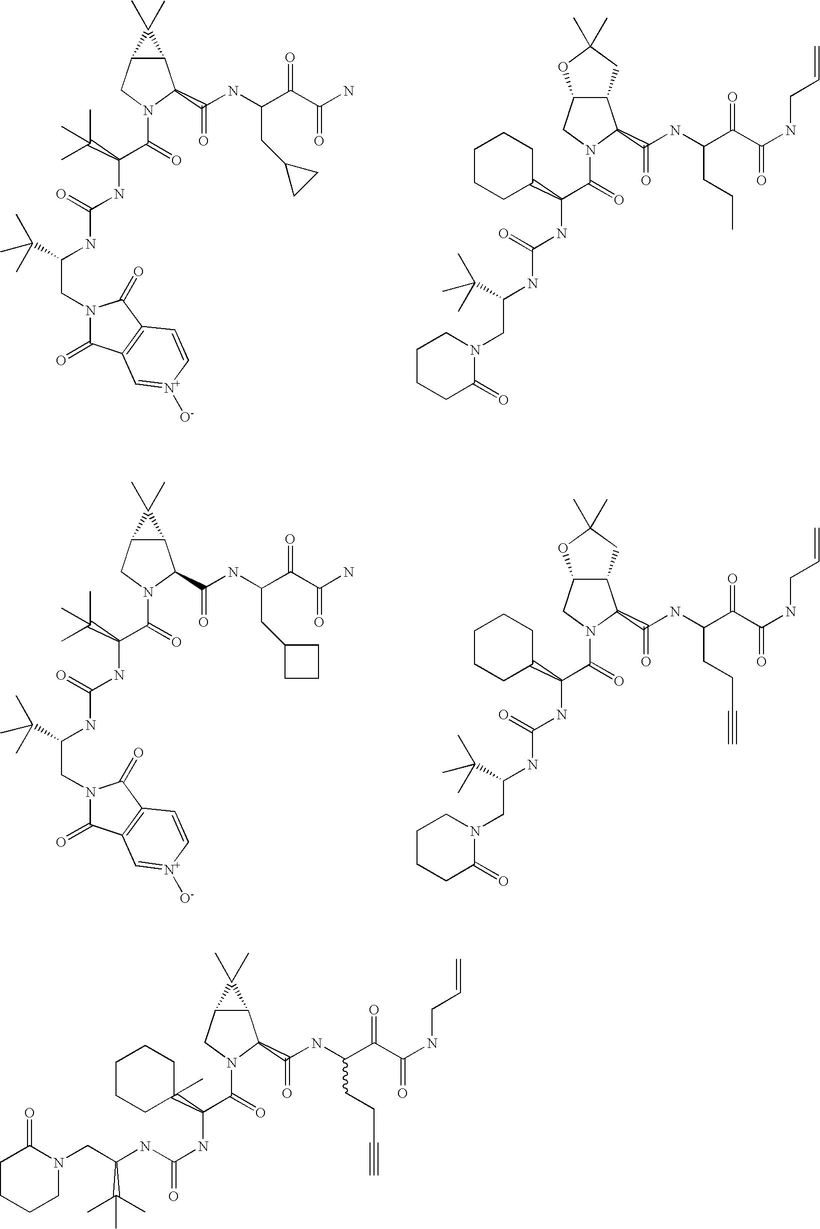 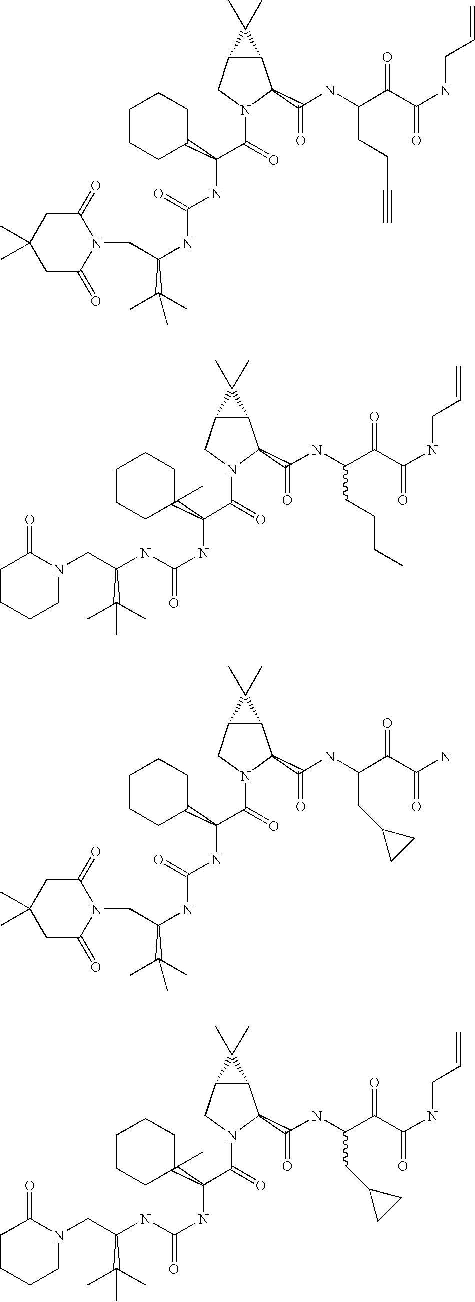 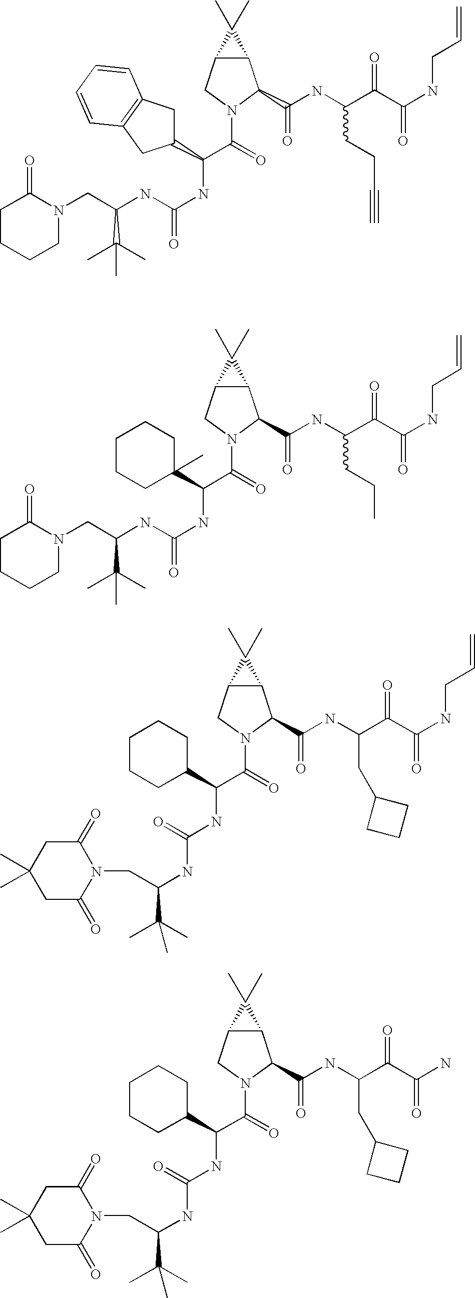 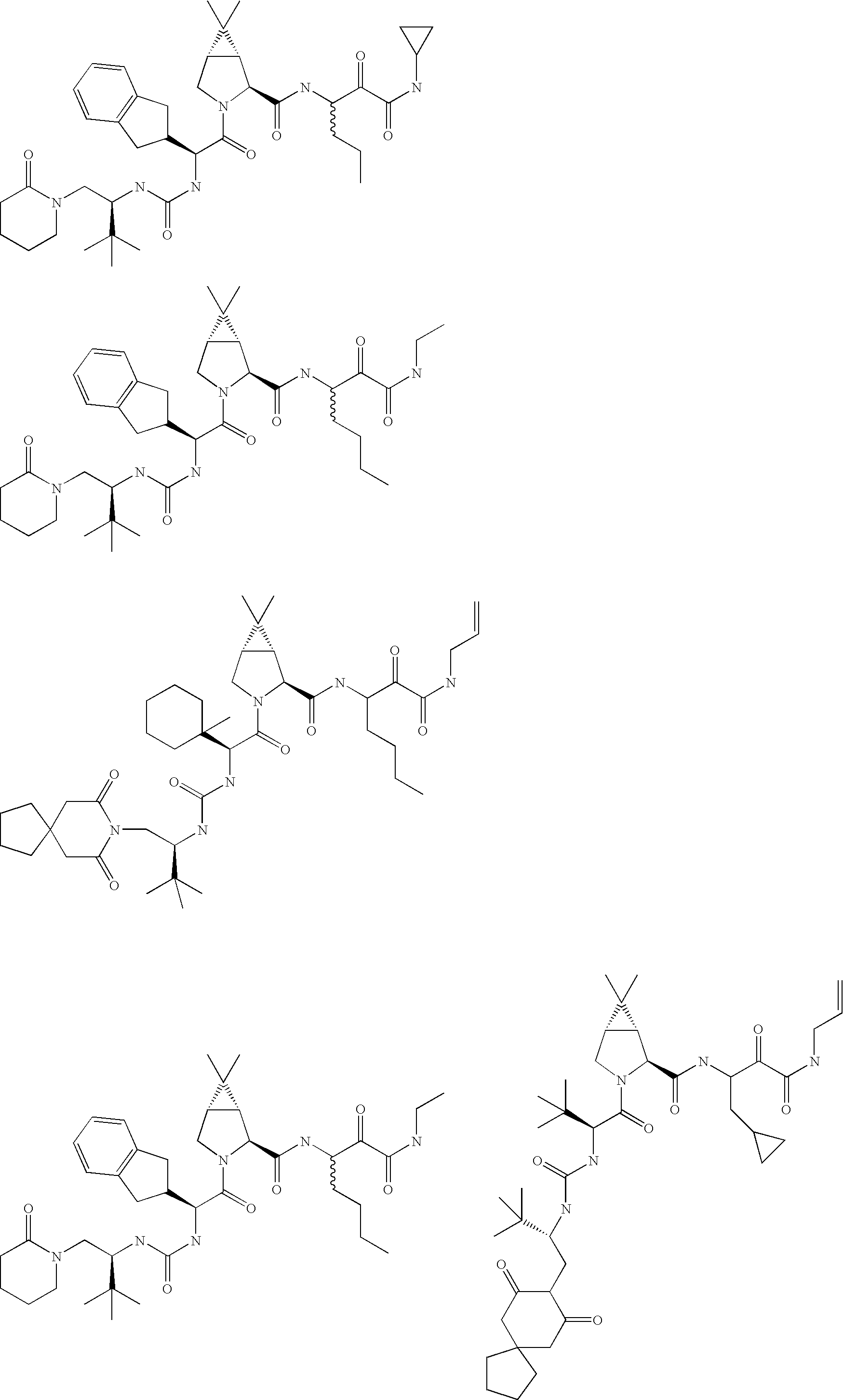 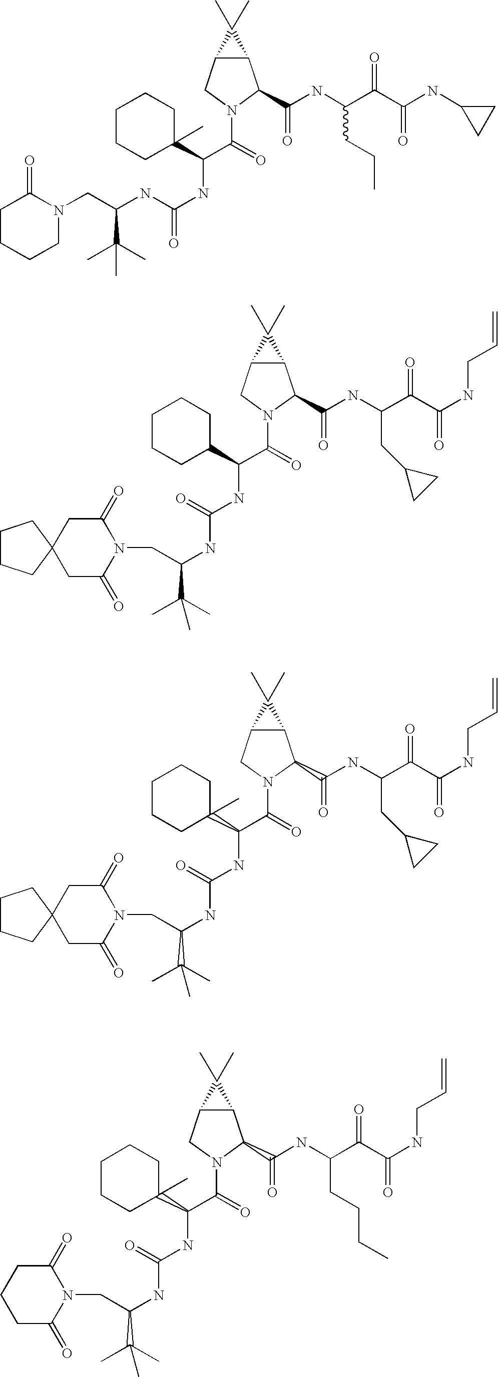 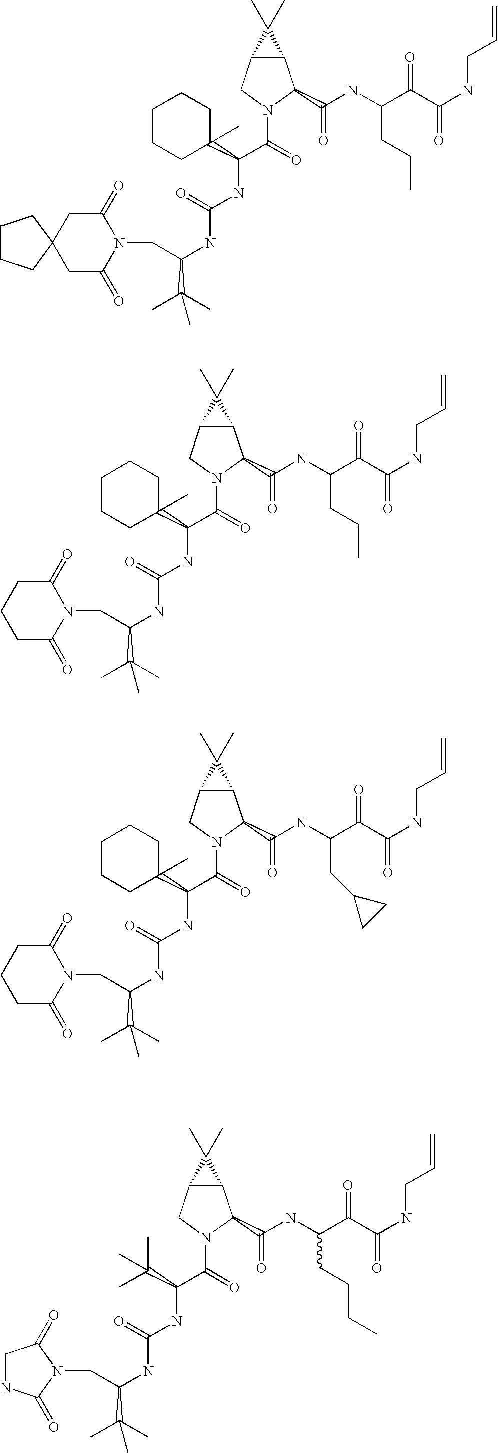 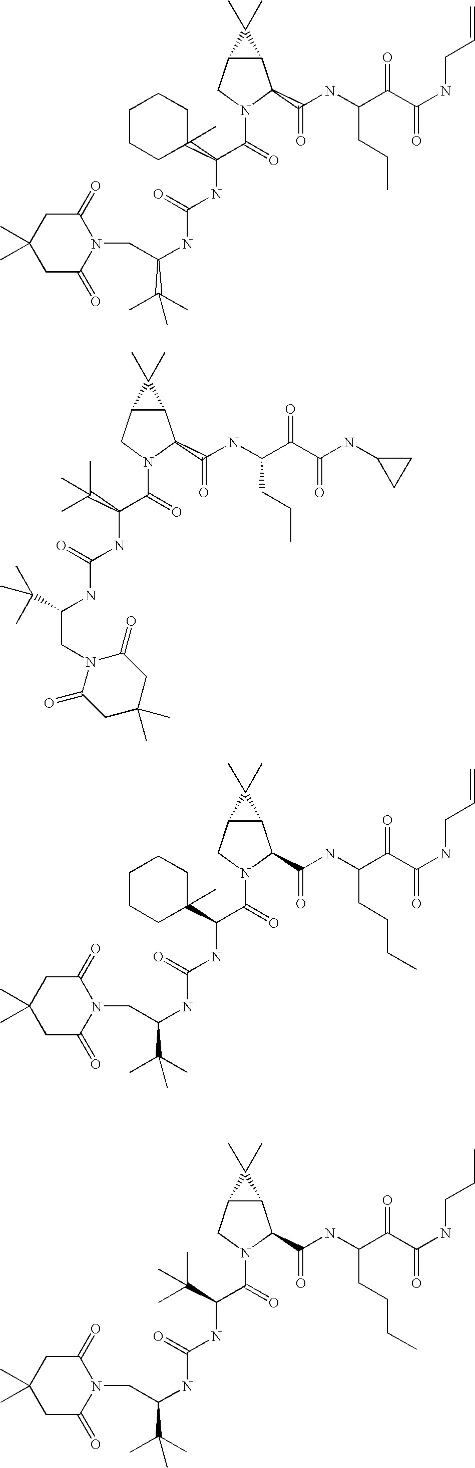 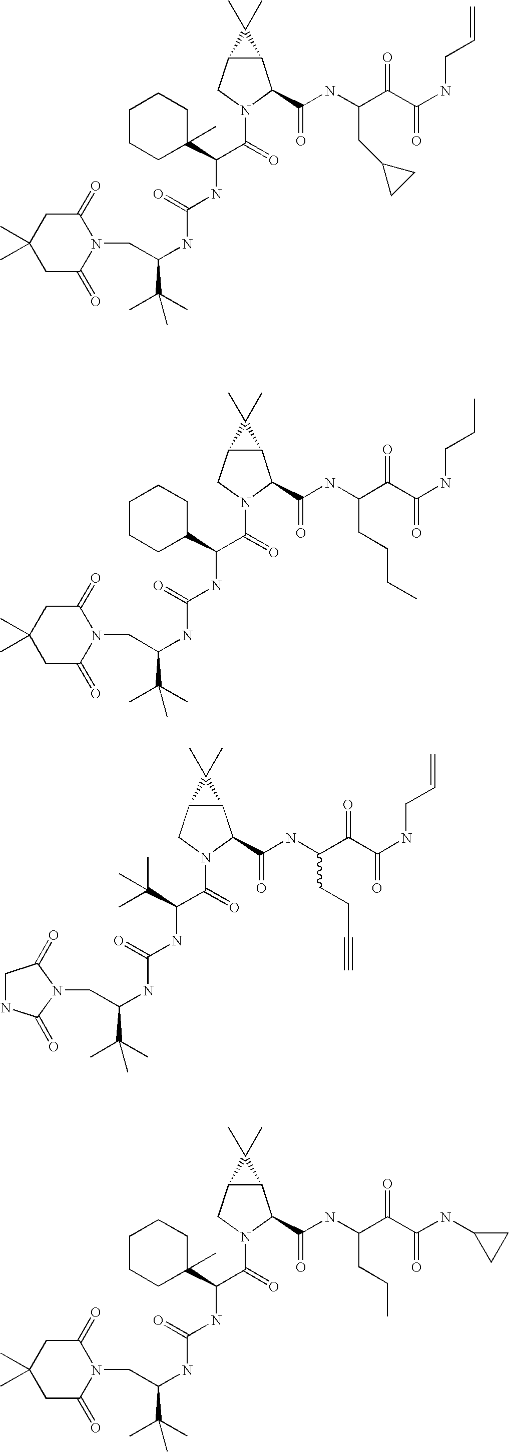 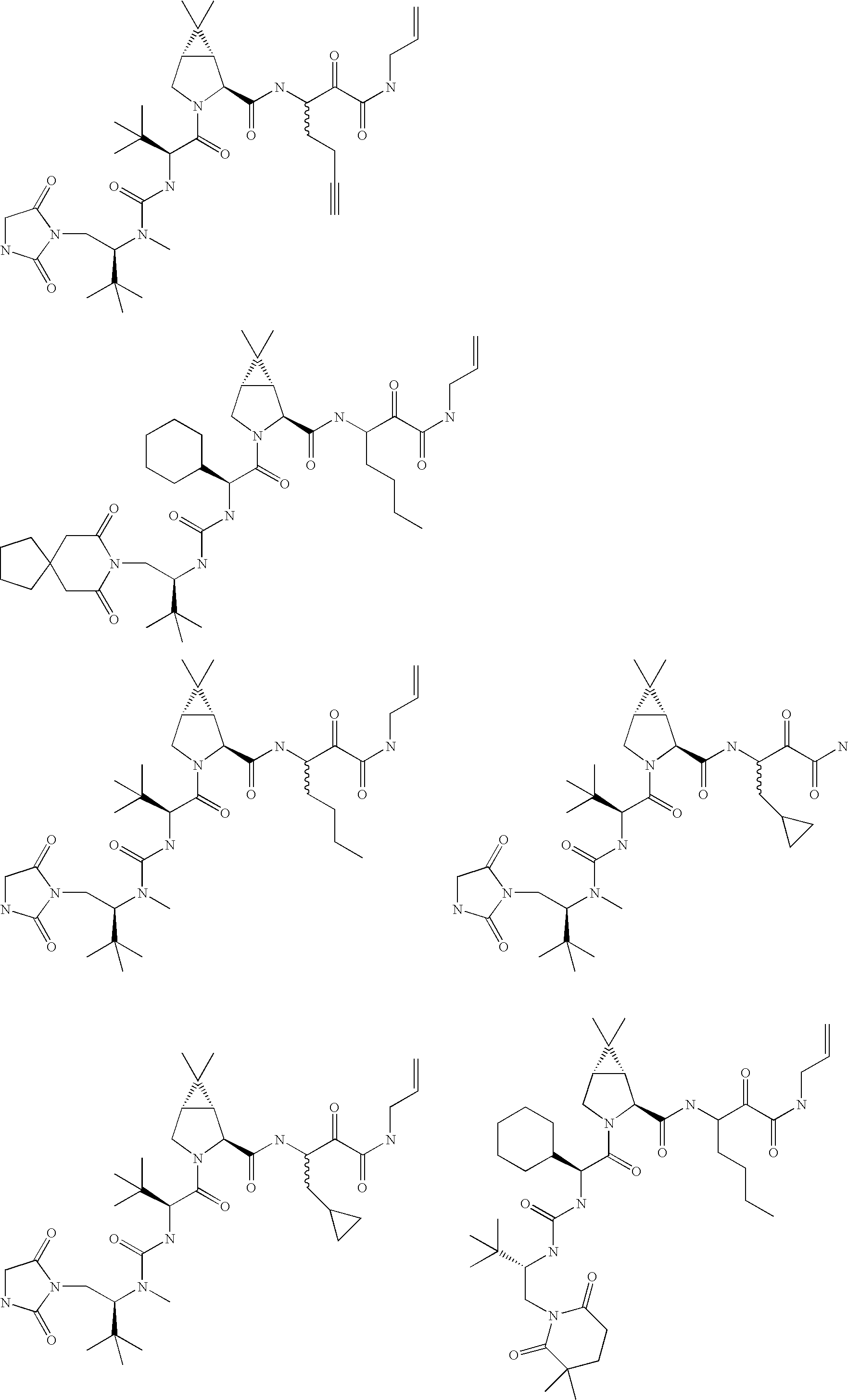 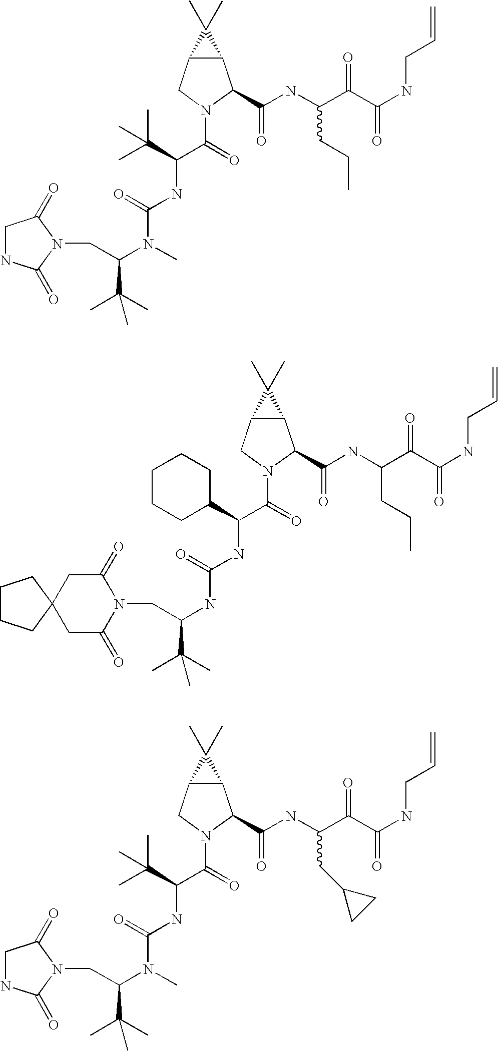
or a pharmaceutically acceptable salt, solvate or ester thereof.

7. A method of inhibiting cathepsin activity in a subject in need thereof comprising administering to said subject an effective amount of at least one compound having the general structure shown in Formula XIV: 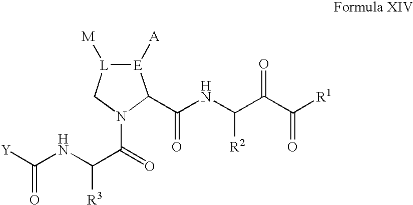
or a pharmaceutically acceptable salt, solvate or ester thereof; wherein:
R¹ is H, OR⁸, NR⁹R¹⁰, or CHR⁹R¹⁰, wherein R⁸, R⁹ and R¹⁰ can be the same or different, each being independently selected from the group consisting of H, alkyl-, alkenyl-, alkynyl-, aryl-, heteroalkyl-, heteroaryl-, cycloalkyl-, heterocyclyl-, arylalkyl-, and heteroarylalkyl;
A and M can be the same or different, each being independently selected from R, OR, NHR, NRR′, SR, SO₂R, and halo;
or A and M are connected to each other such that the moiety: 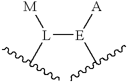
shown above in Formula XIV forms either a three, four, six, seven or eight-membered cycloalkyl, a four to eight-membered heterocyclyl, a six to ten-membered aryl, or a five to ten-membered heteroaryl;
E is C(H) or C(R);
L is C(H), C(R), CH₂C(R), or C(R)CH₂;
R, R′, R², and R³ can be the same or different, each being independently selected from the group consisting of H, alkyl, heteroalkyl, alkenyl, heteroalkenyl, alkynyl, heteroalkynyl, cycloalkyl, heterocyclyl, aryl, arylalkyl, heteroaryl, and heteroarylalkyl, or alternately R and R′ in NRR′ are connected to each other such that NRR′ forms a four to eight-membered heterocyclyl;
and Y is selected from the following moieties: 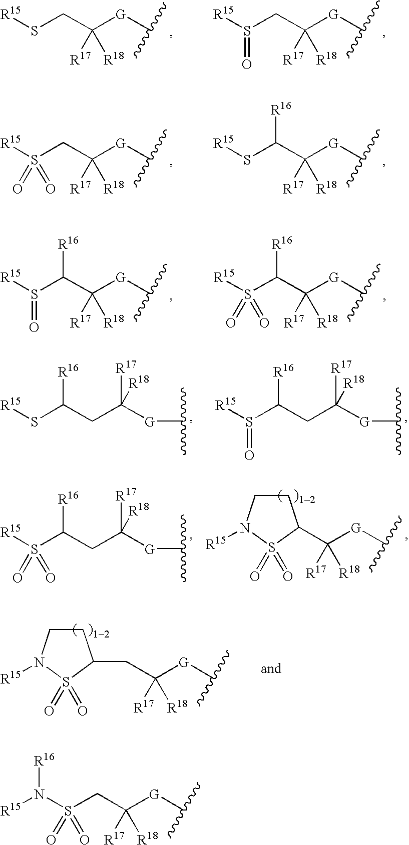
wherein G is NH or O; and R¹⁵, R¹⁶, R¹⁷ and R¹⁸ can be the same or different, each being independently selected from the group consisting of H, alkyl, heteroalkyl, alkenyl, heteroalkenyl, alkynyl, heteroalkynyl, cycloalkyl, heterocyclyl, aryl, and heteroaryl, or alternately, (i) R¹⁵ and R¹⁶ are connected to each other to form a four to eight-membered cyclic structure, and (ii) likewise, independently R¹⁷ and R¹⁸ are connected to each other to form a three to eight-membered cycloalkyl or heterocyclyl;
wherein each of said alkyl, aryl, heteroaryl, cycloalkyl or heterocyclyl can be unsubstituted or optionally independently substituted with one or more moieties selected from the group consisting of: hydroxy, alkoxy, aryloxy, thio, alkylthio, arylthio, amino, amido, alkylamino, arylamino, alkylsulfonyl, arylsulfonyl, sulfonamido, alkylsulfonamido, arylsulfonamido, alkyl, aryl, heteroaryl, keto, carboxy, carbalkoxy, carboxamido, alkoxycarbonylamino, alkoxycarbonyloxy, alkylureido, arylureido, halo, cyano, and nitro.

8. The method of claim 7, wherein said compound is selected from the group consisting of: 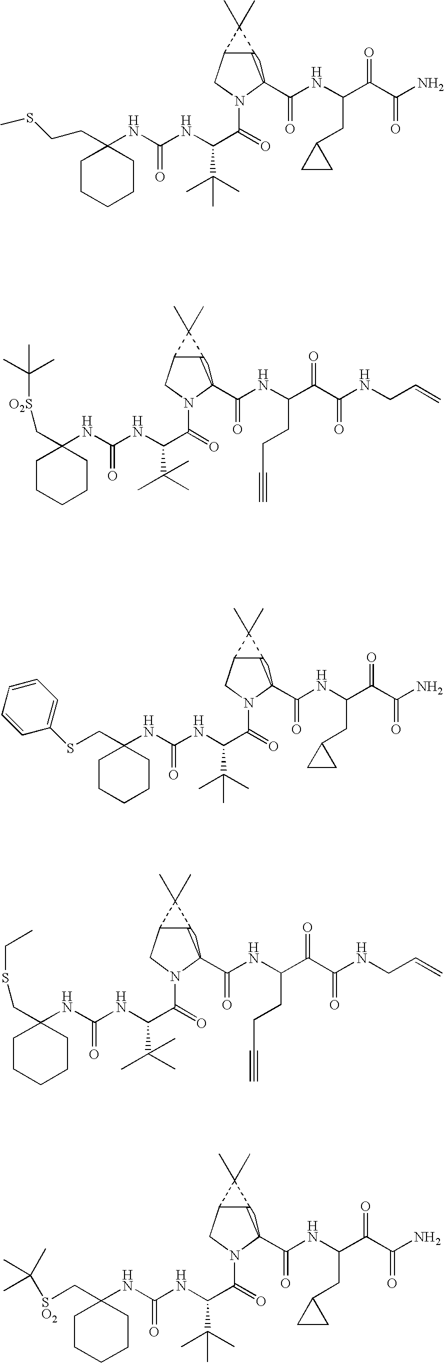 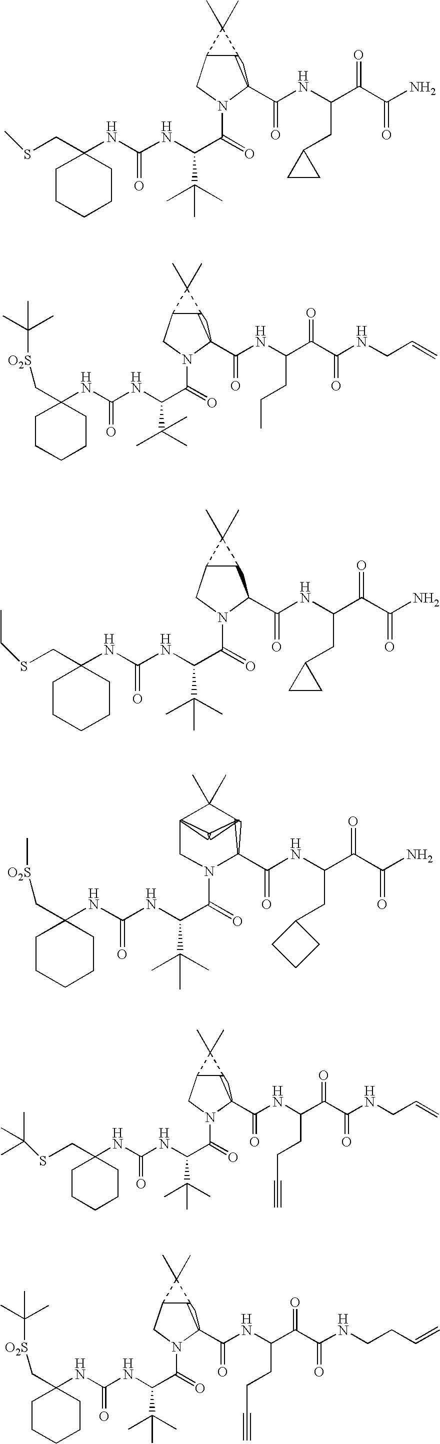 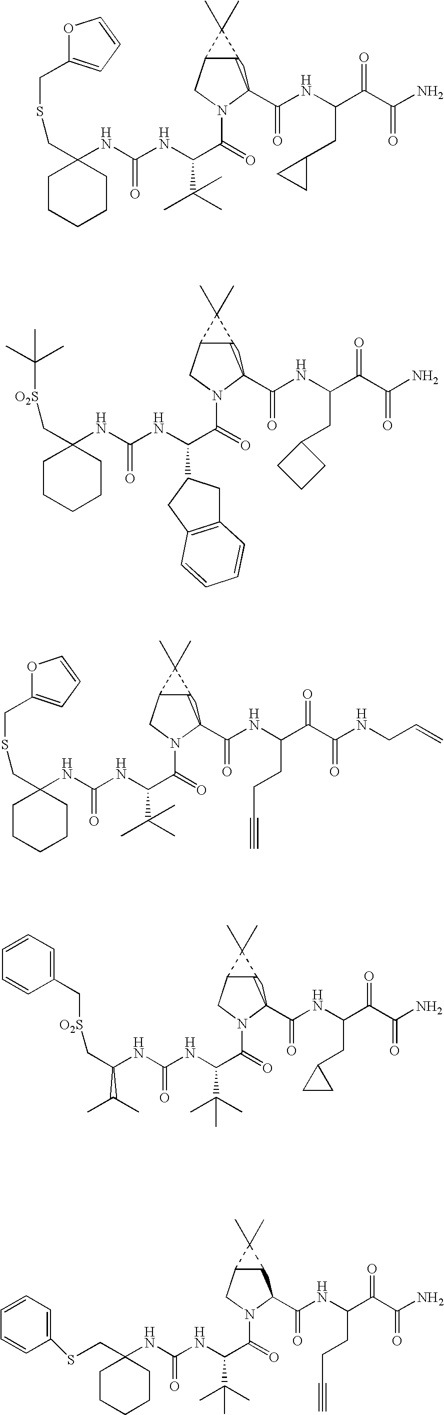 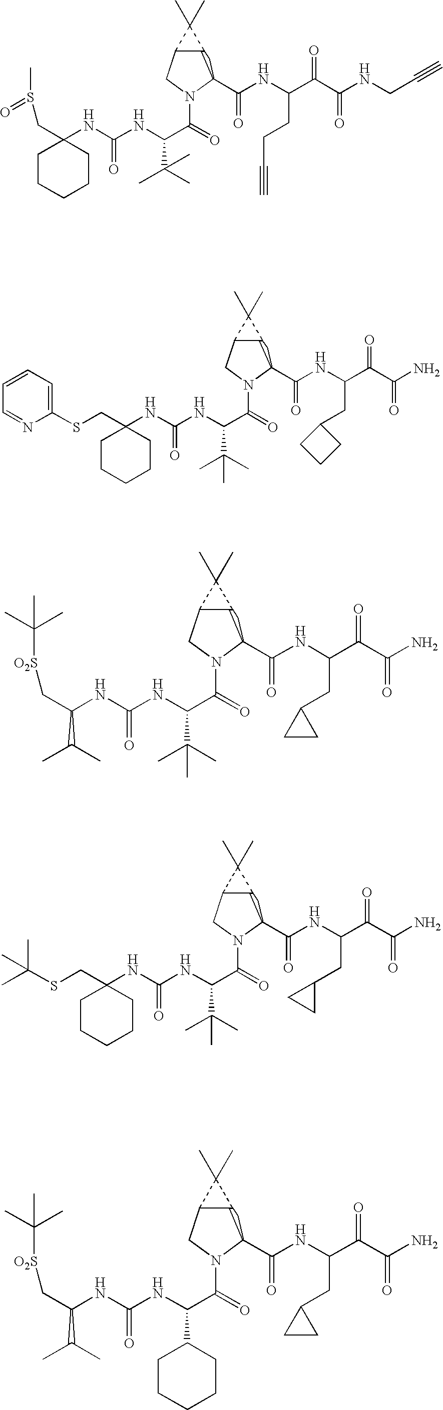 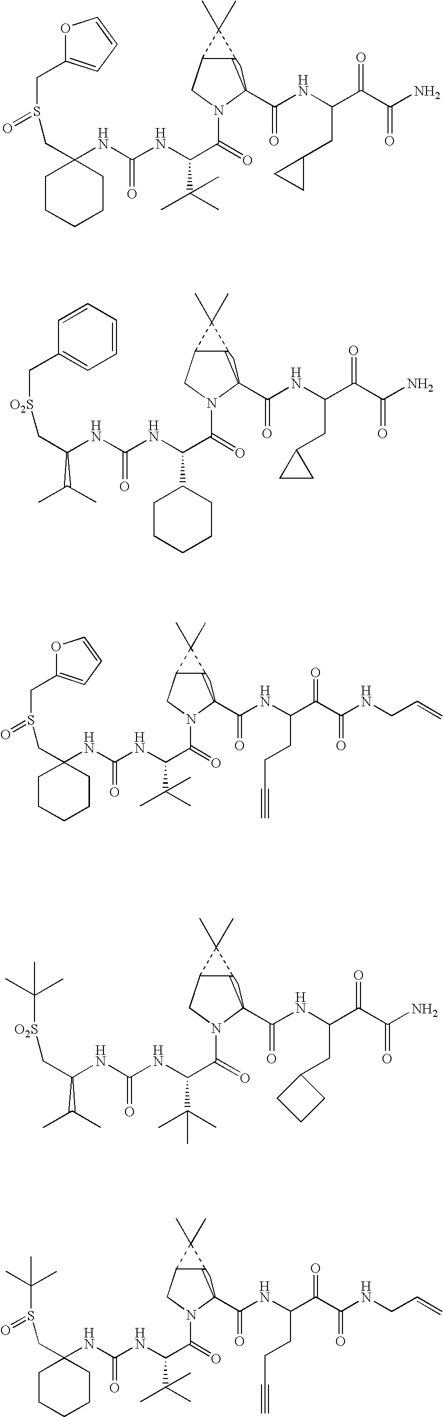 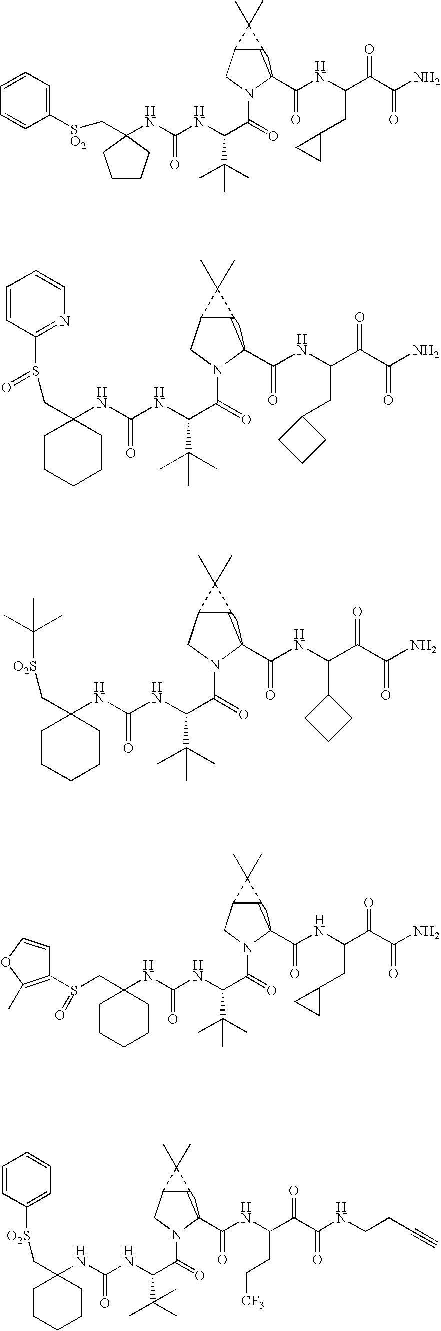 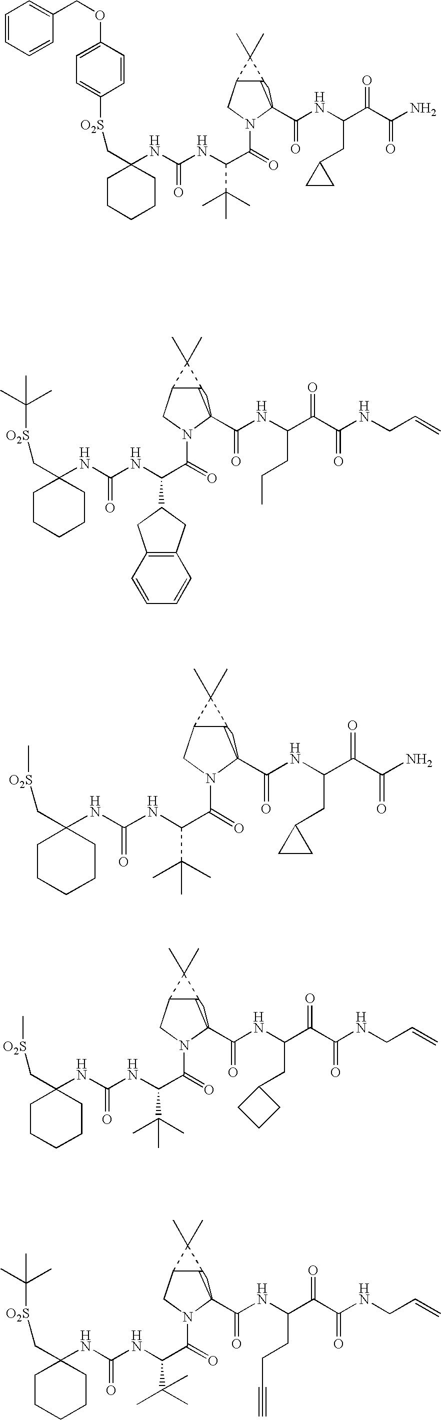 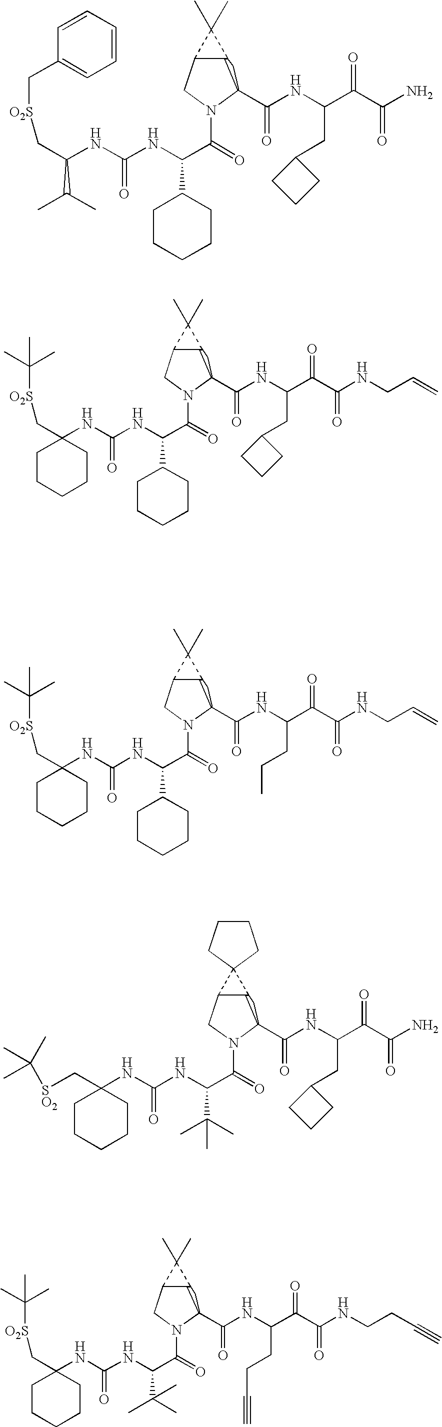 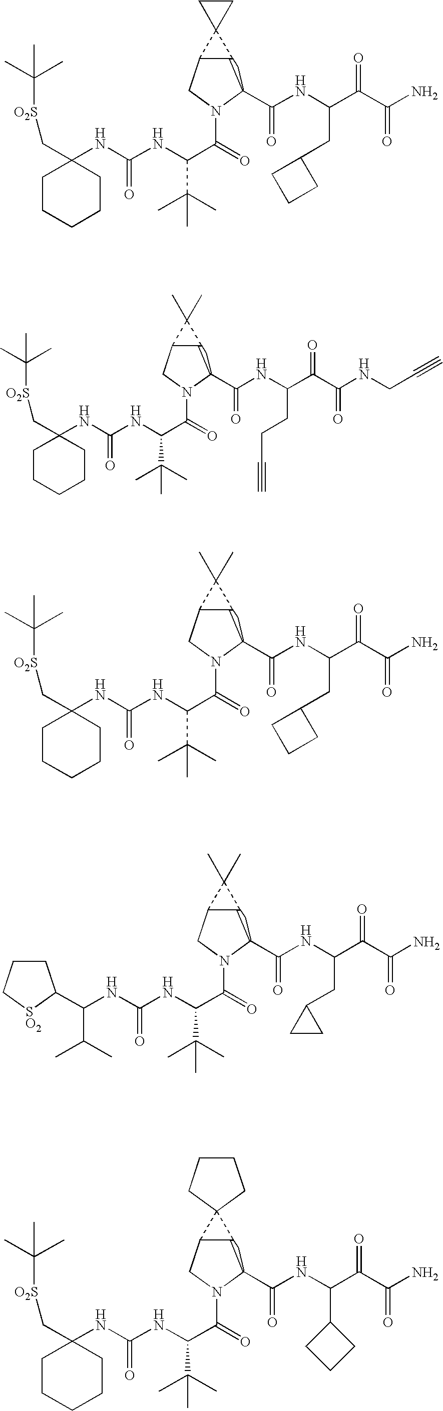 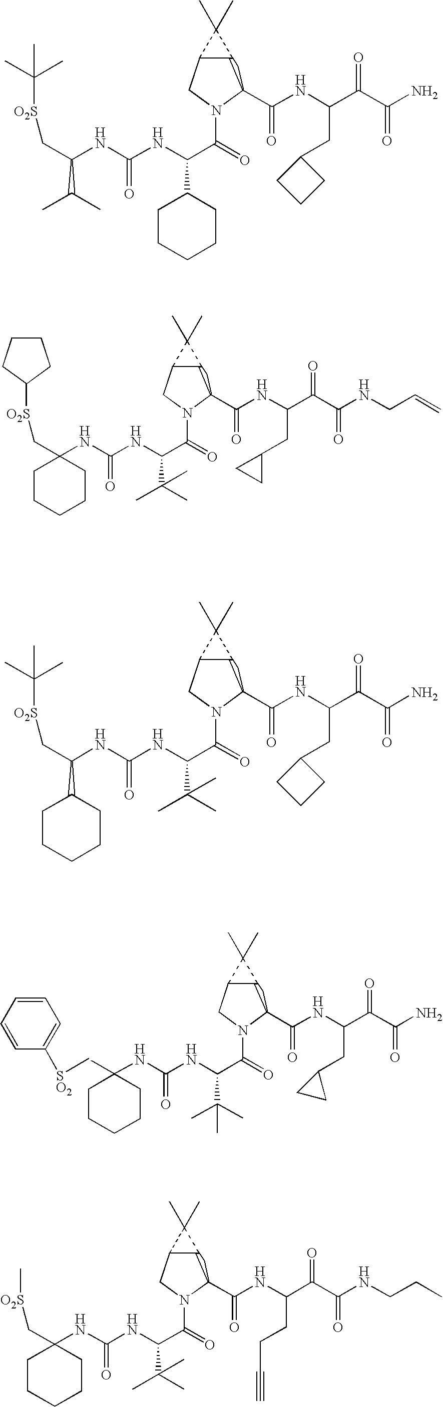 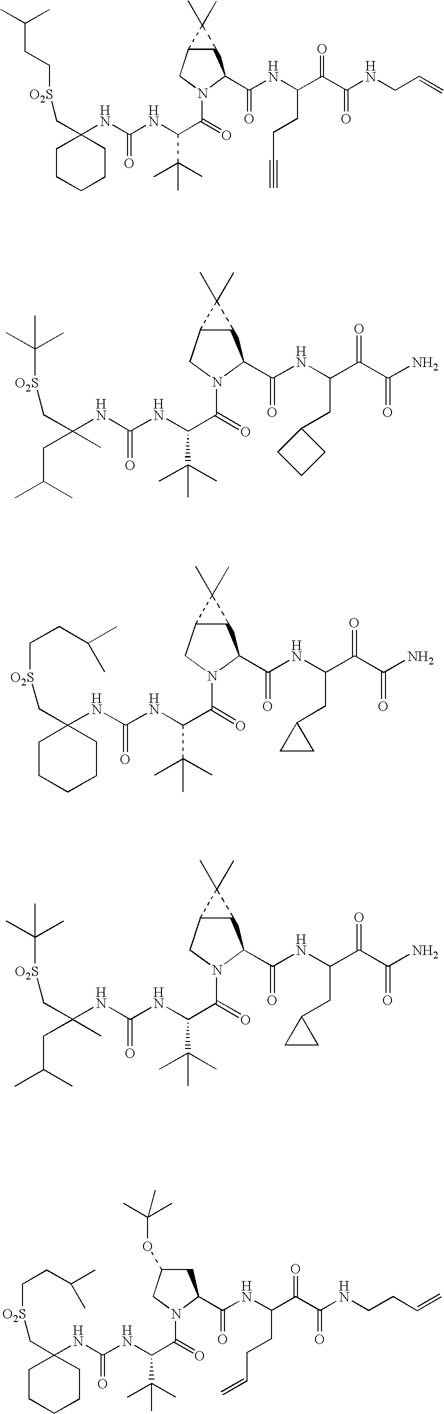 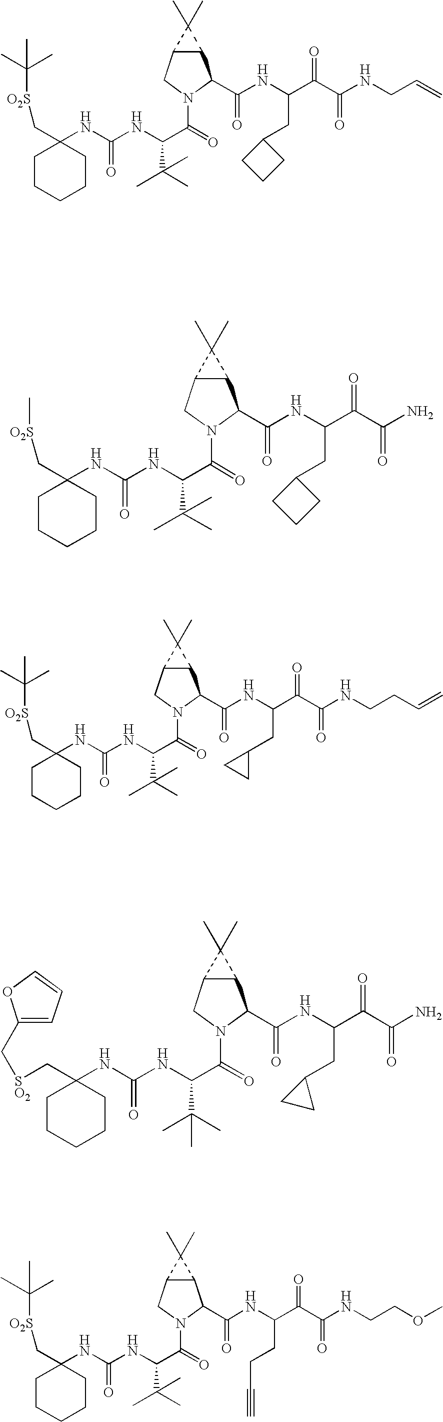 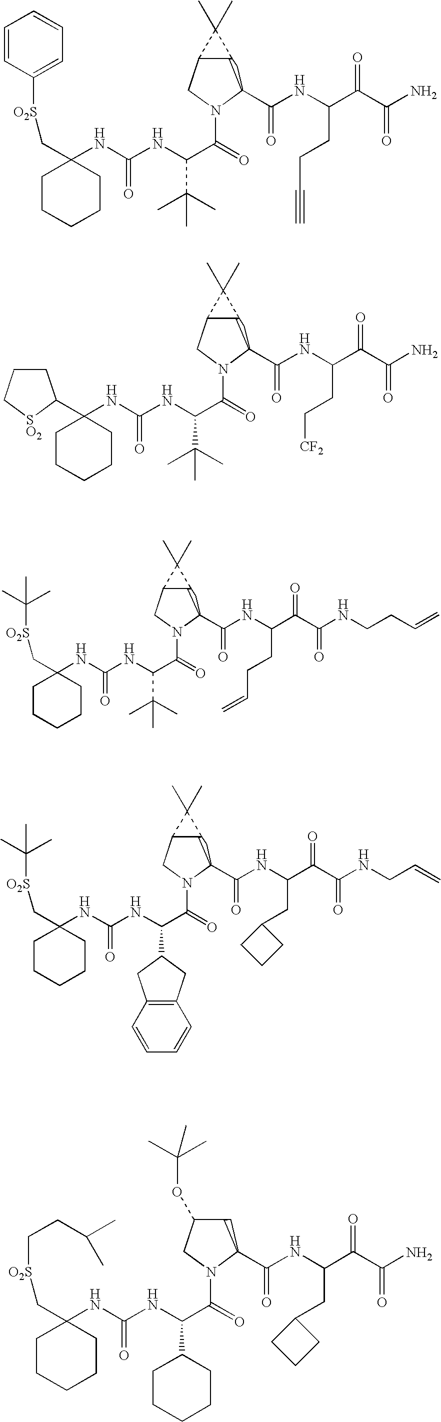 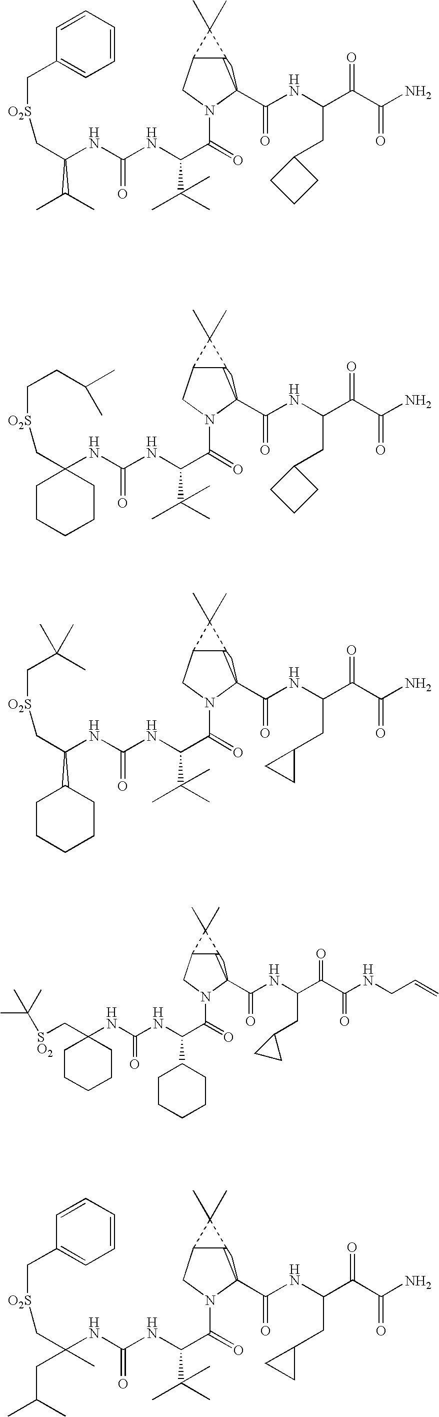 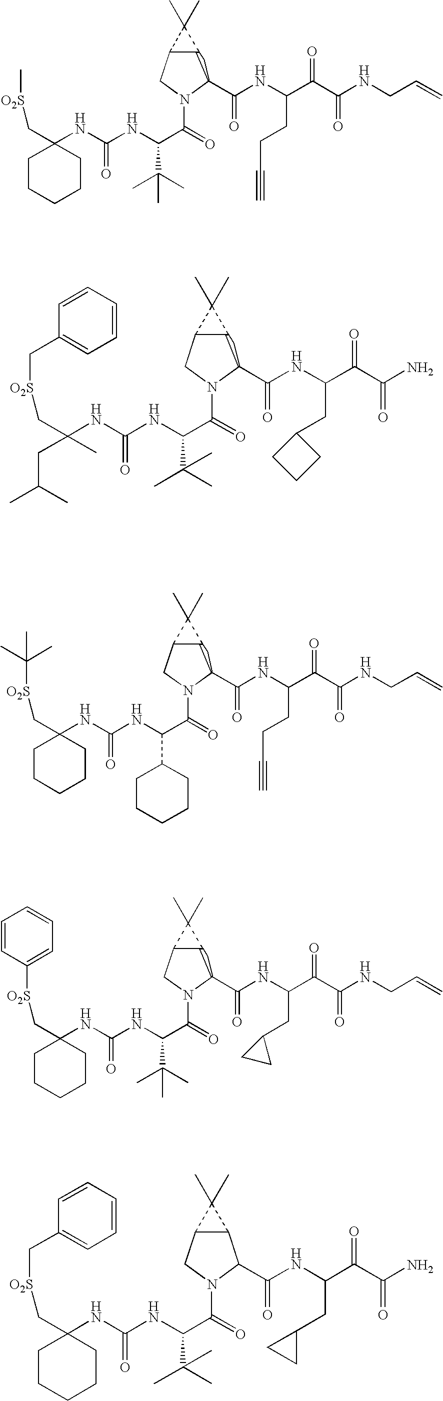 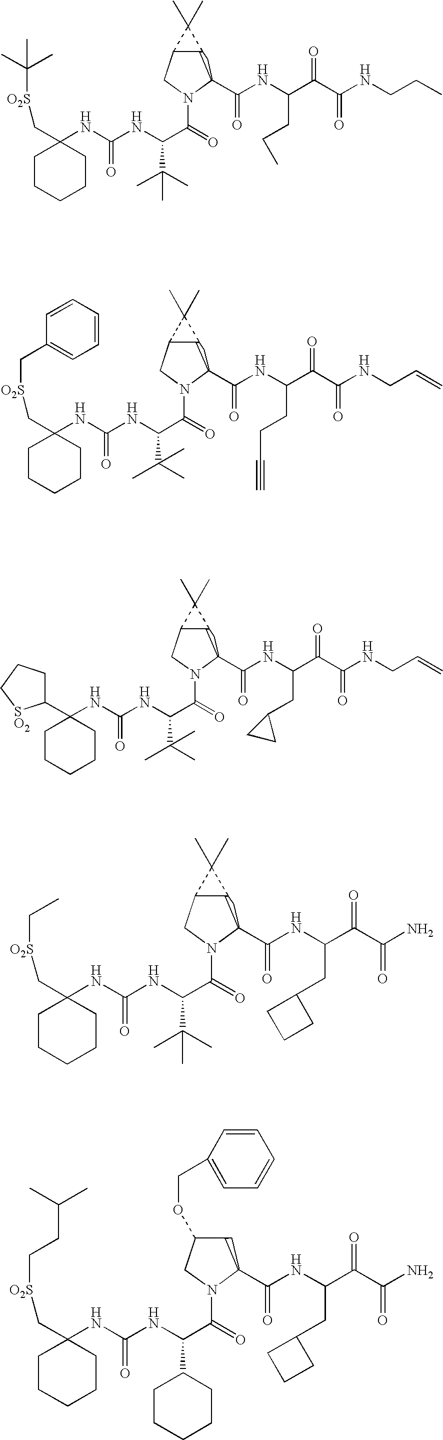 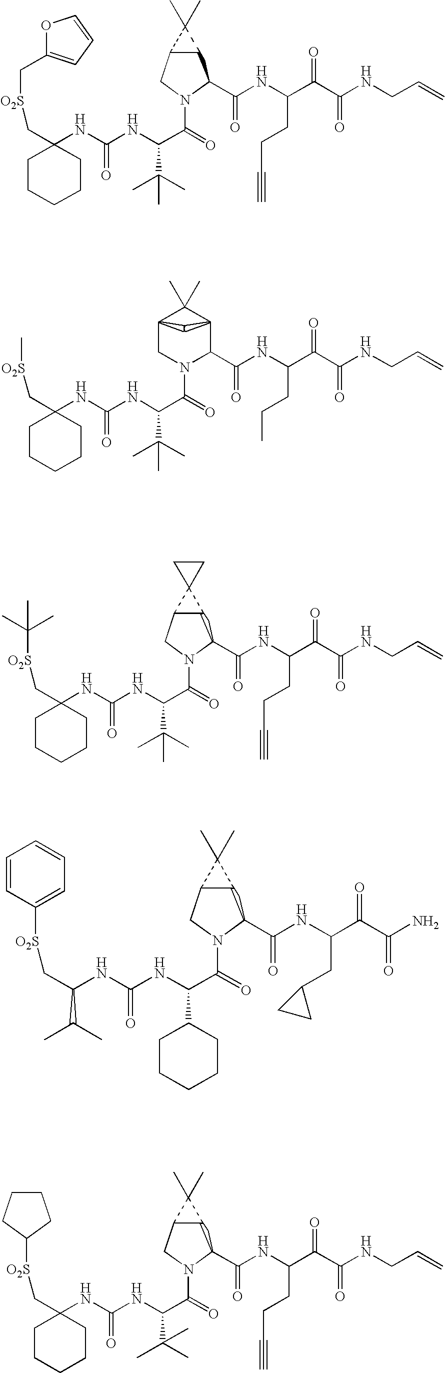 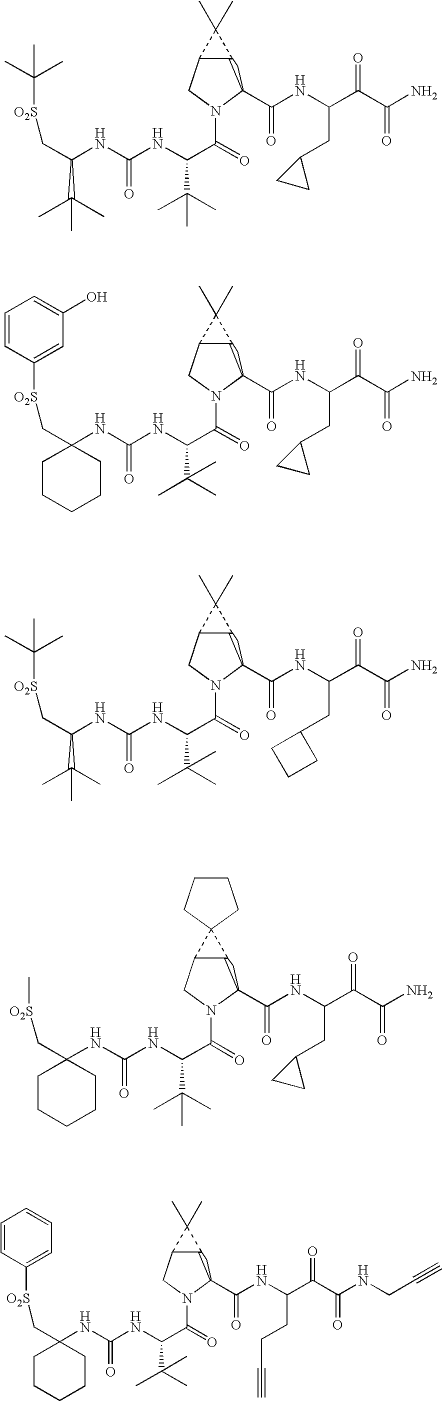 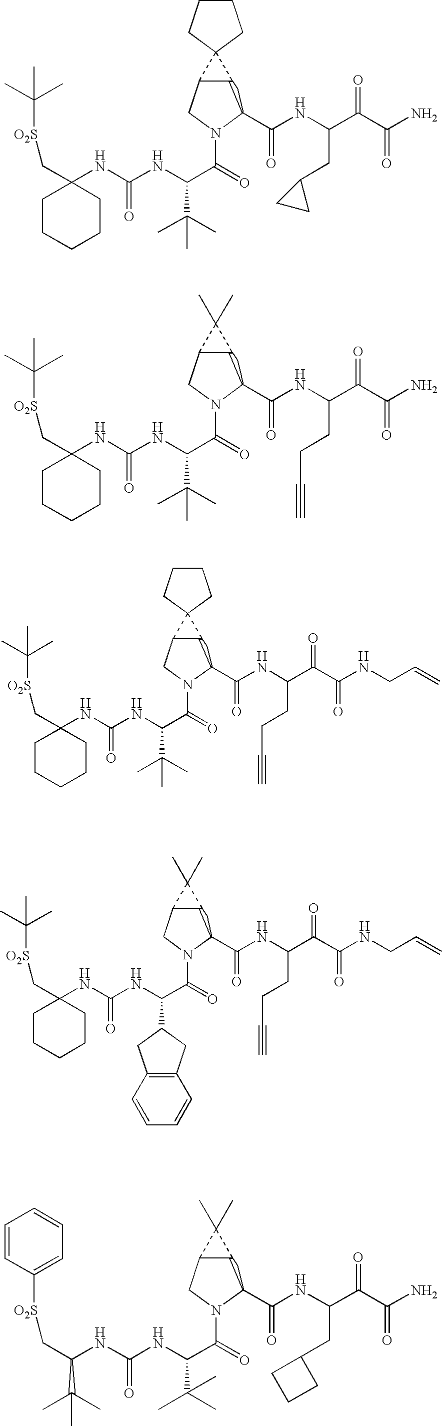 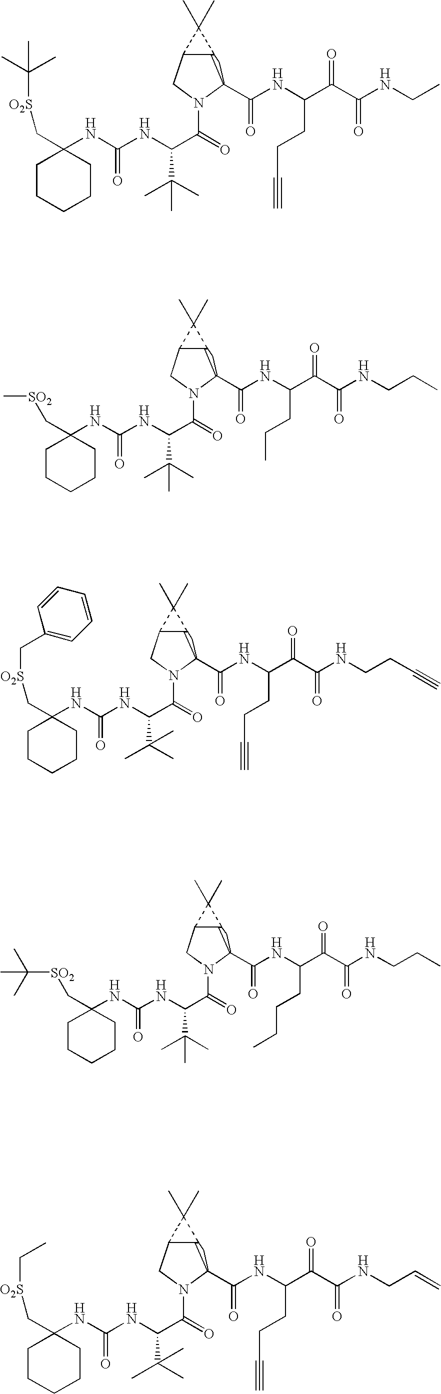 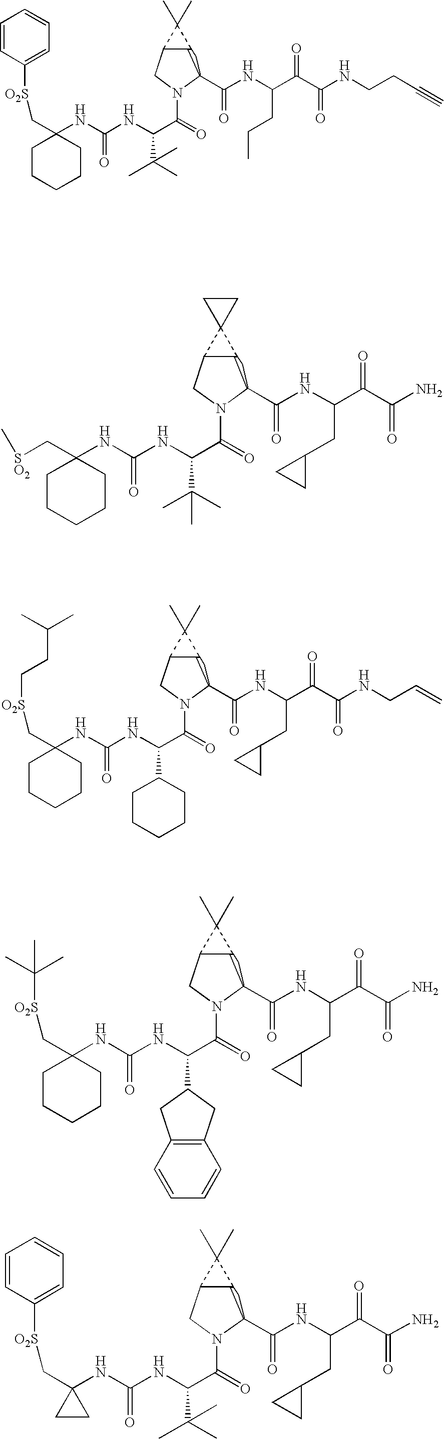 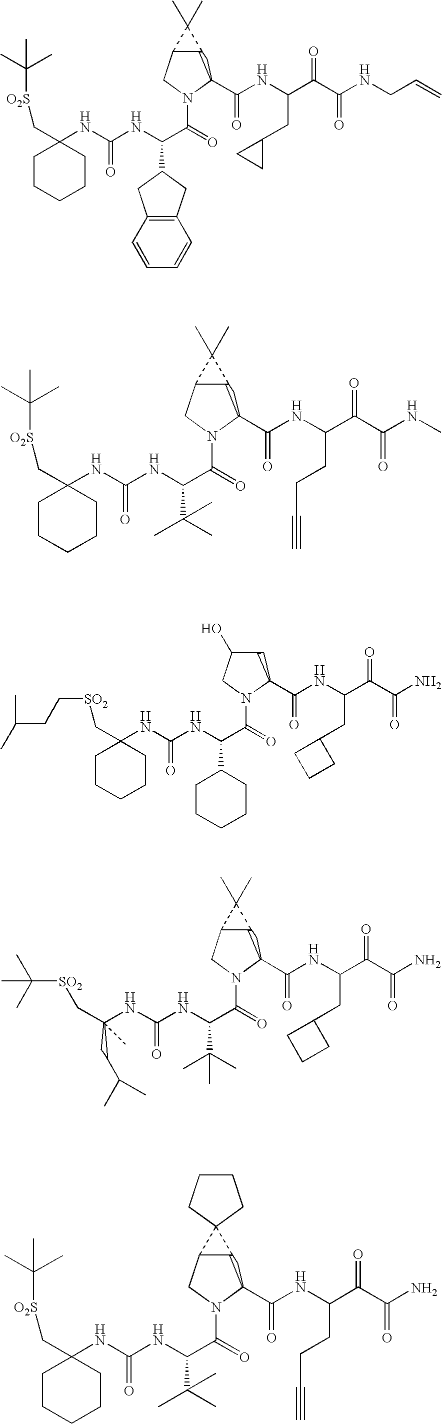 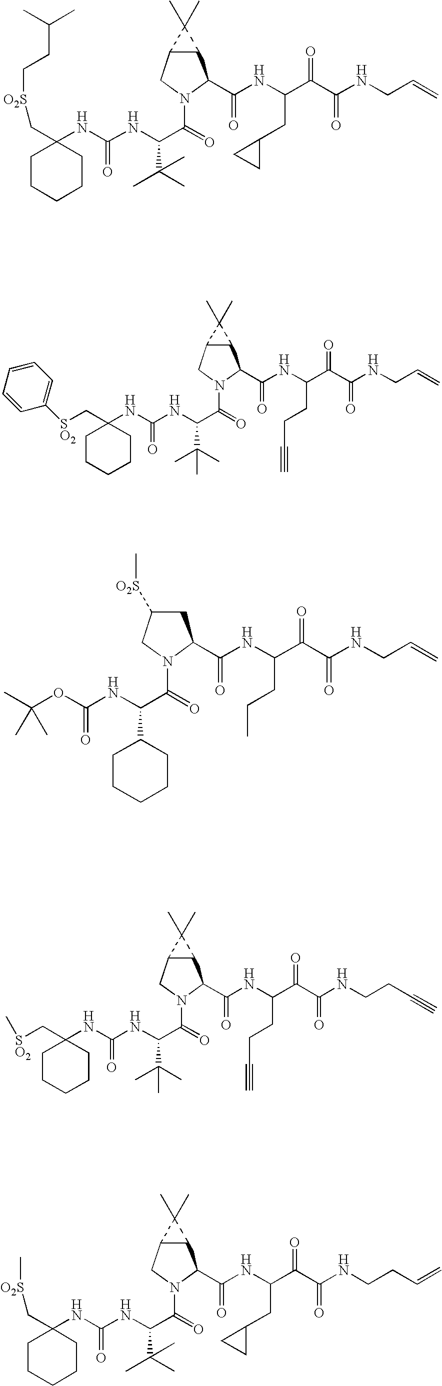 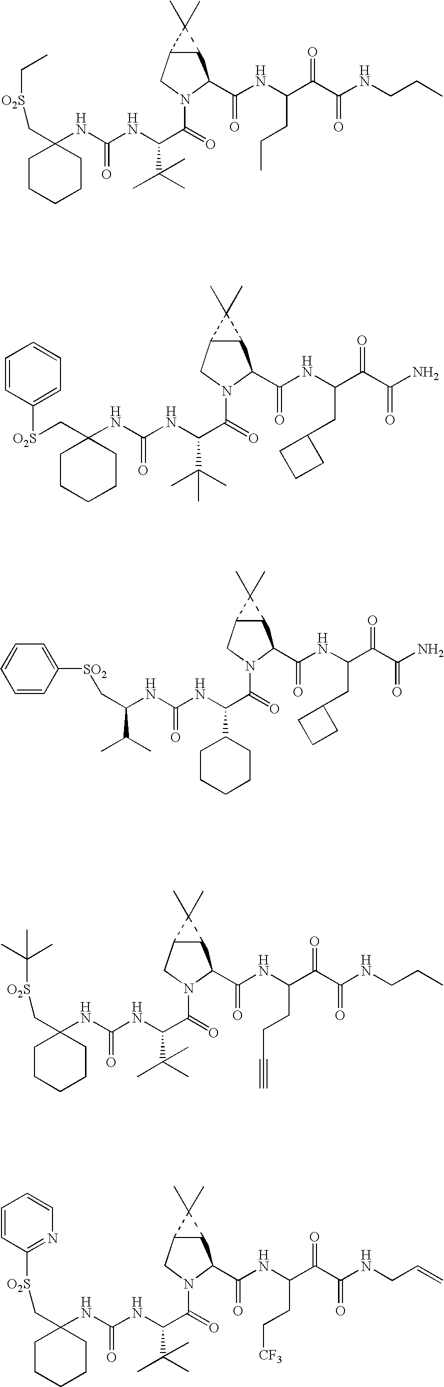 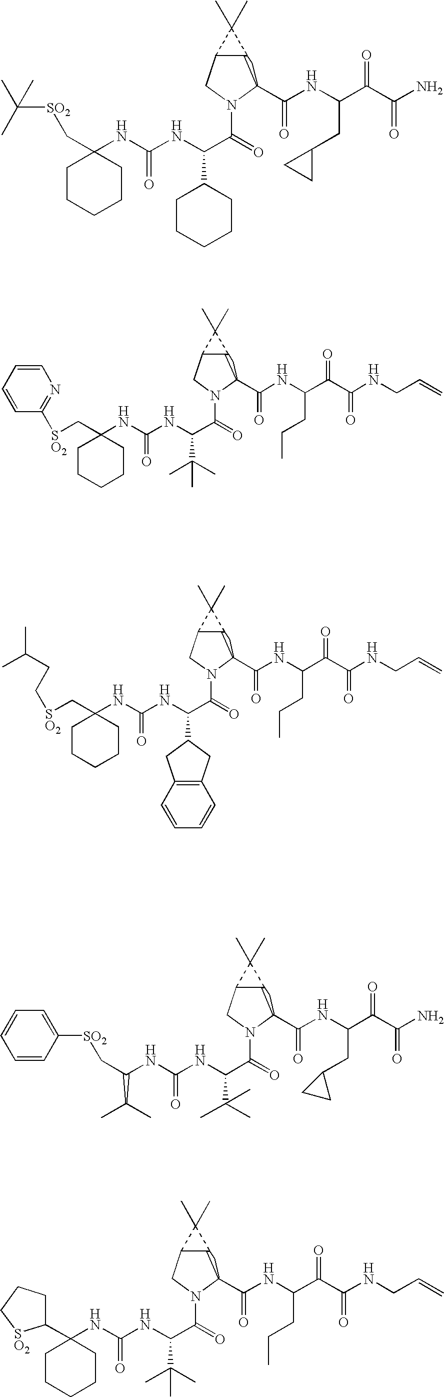 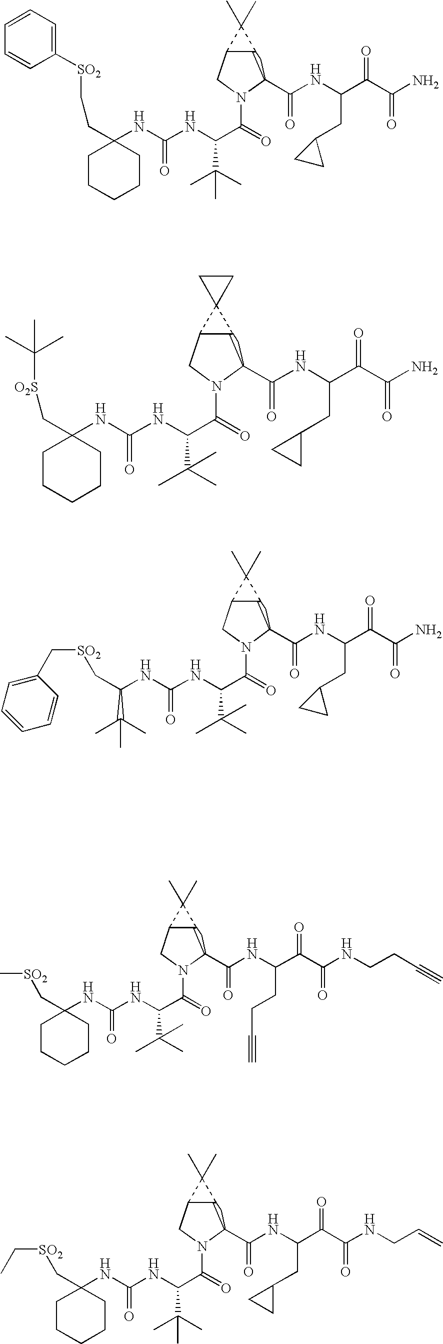 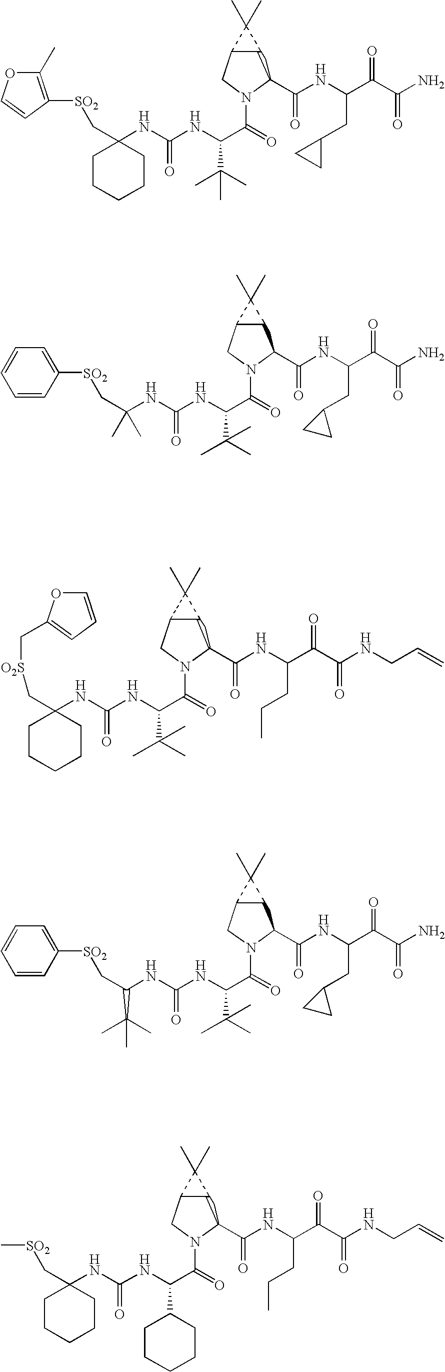 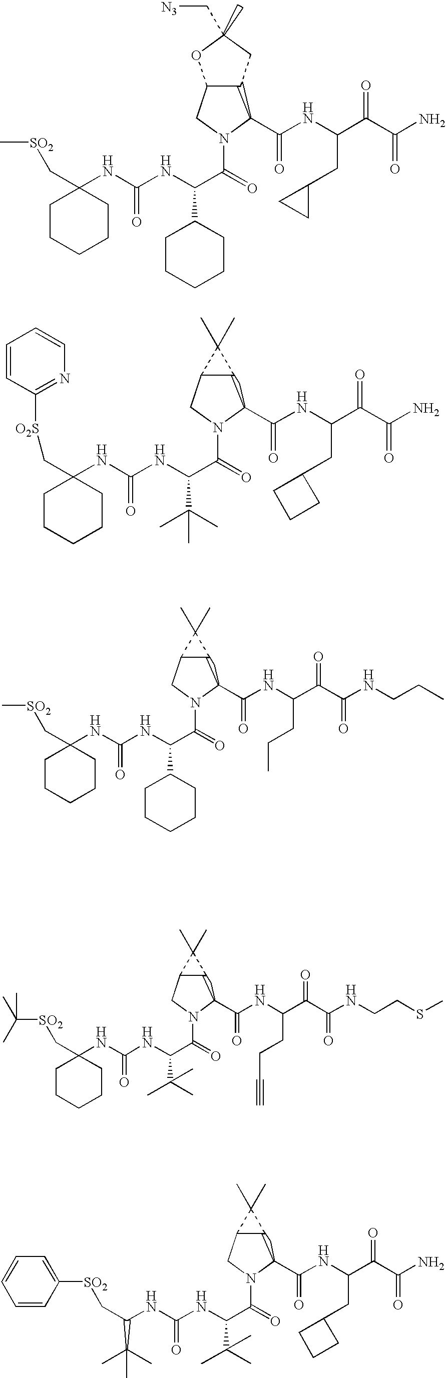 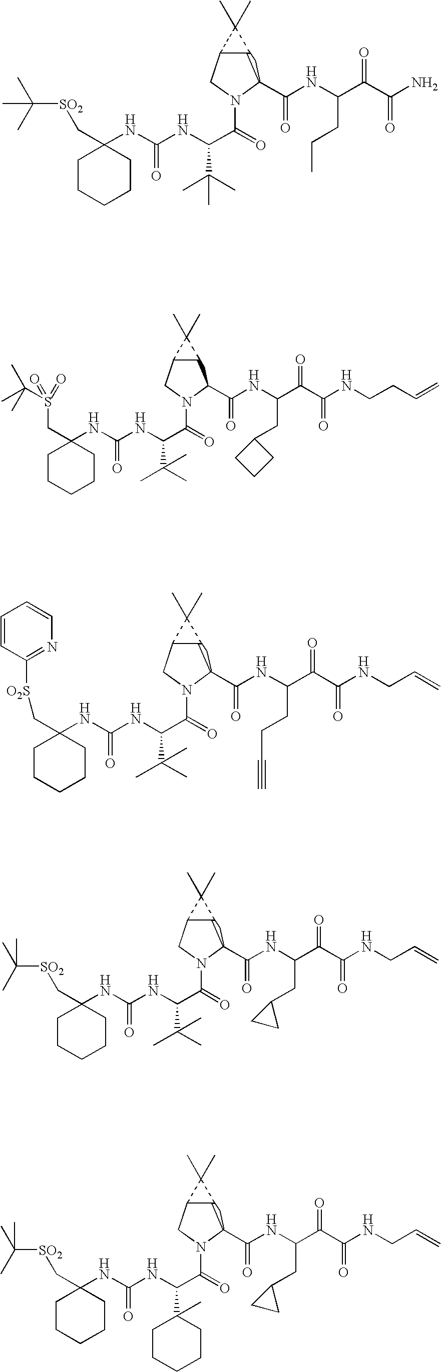 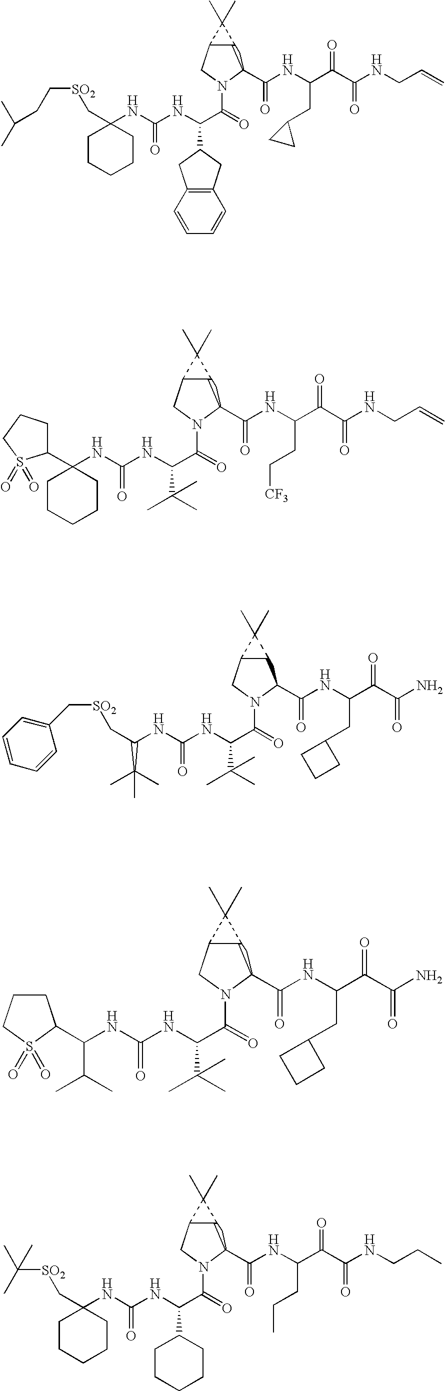 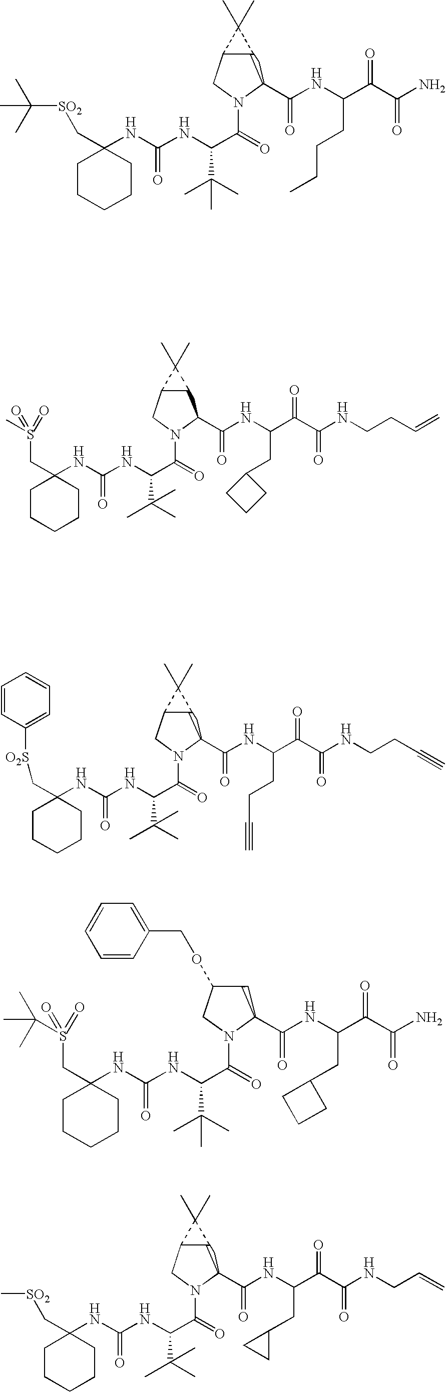 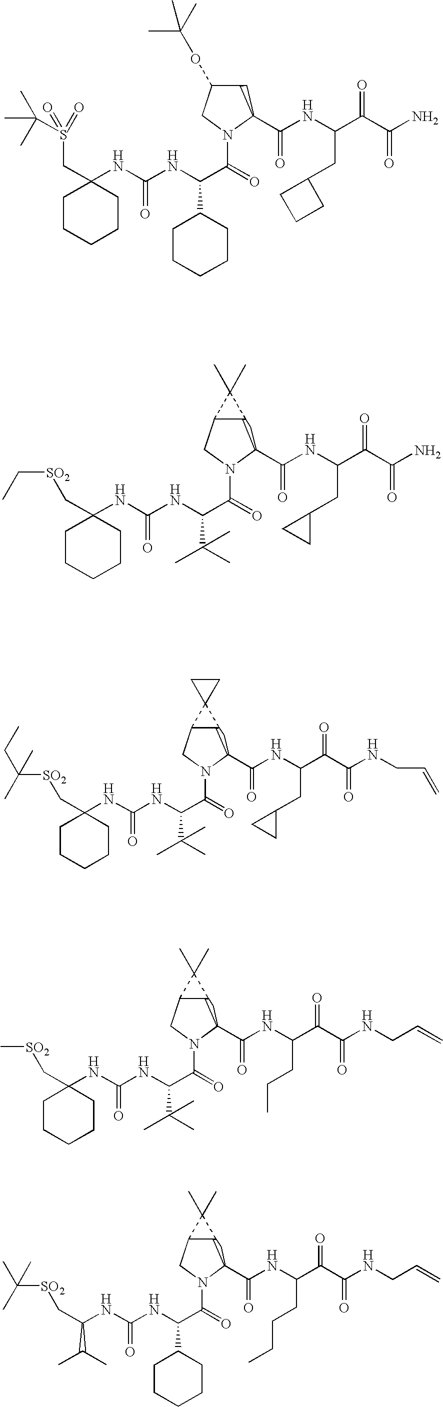 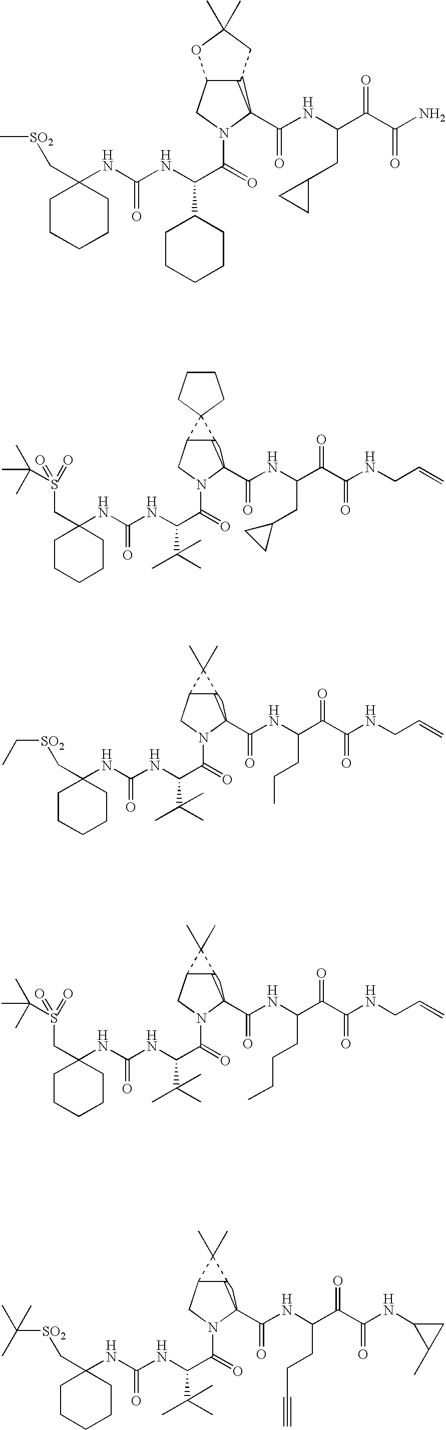 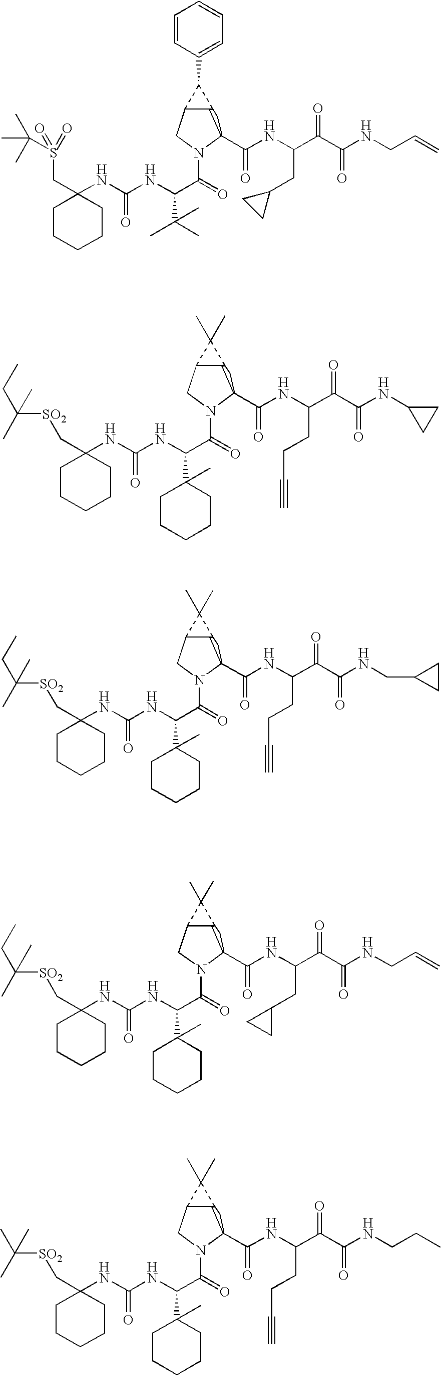 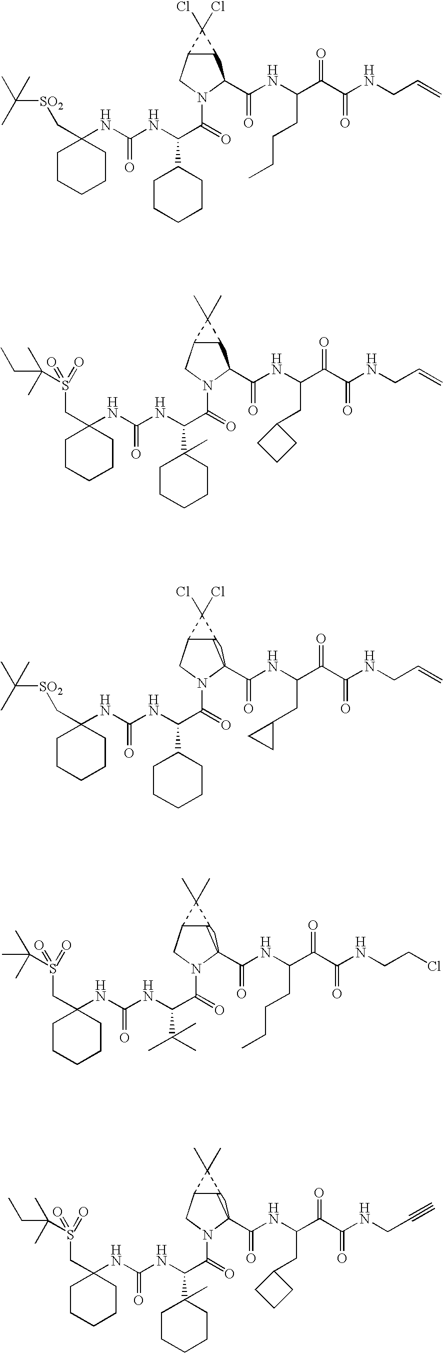 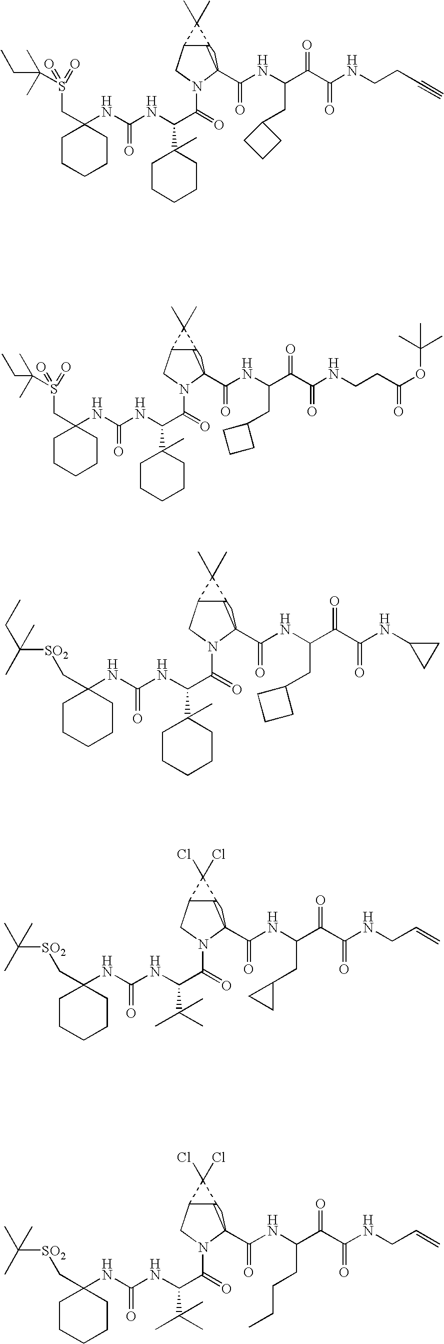 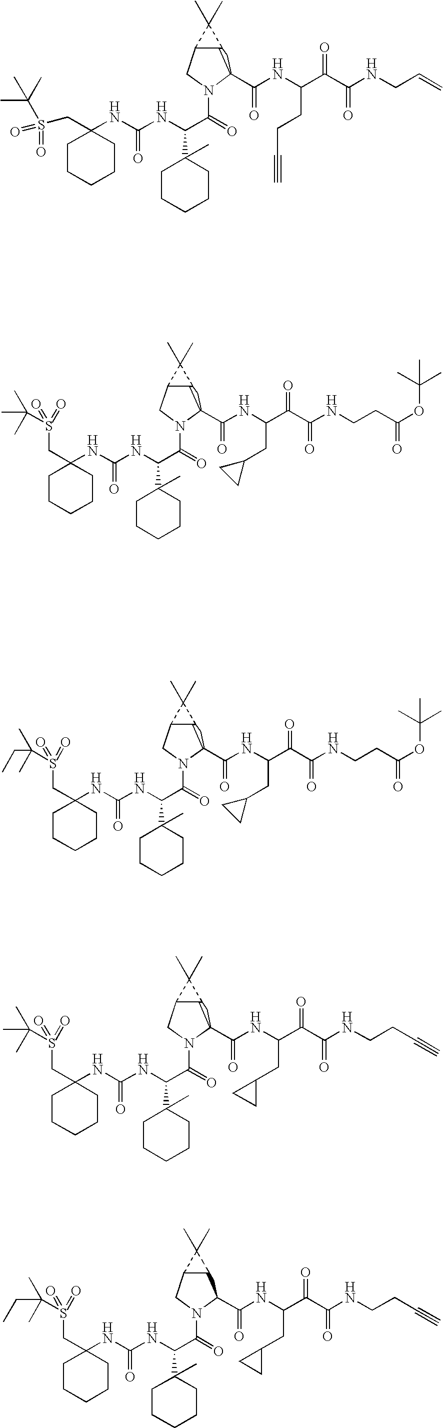 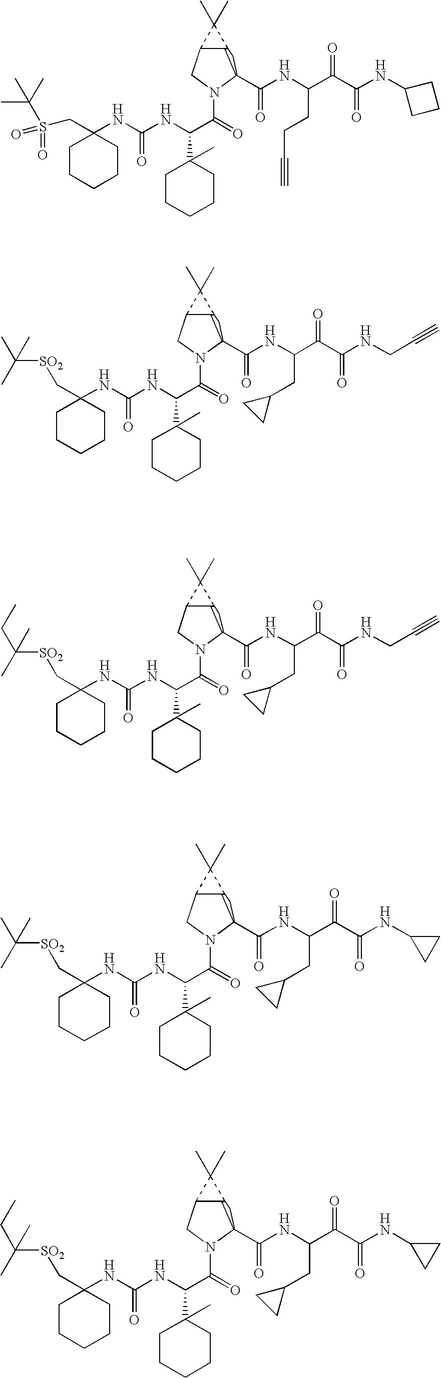 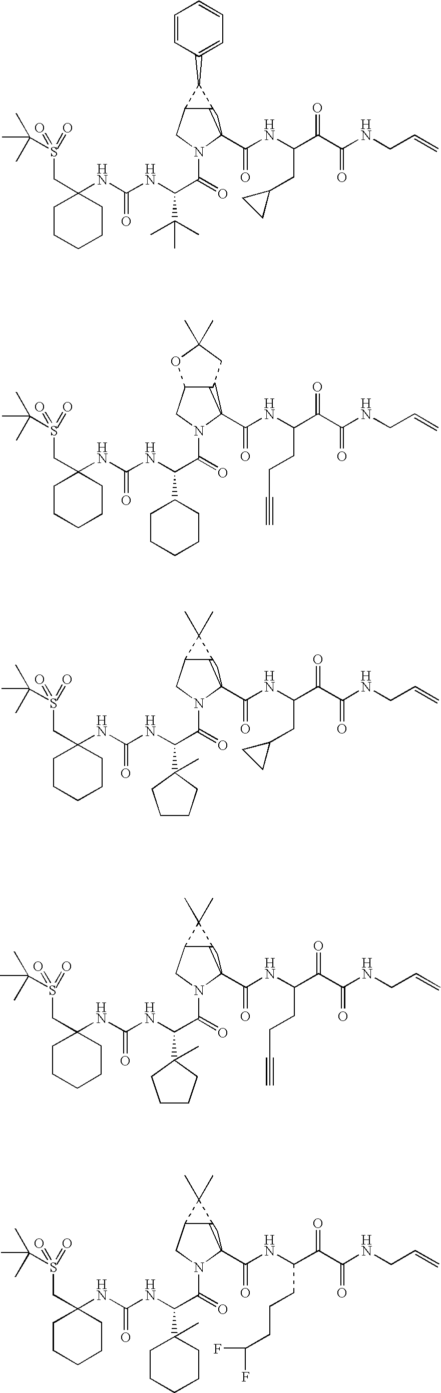 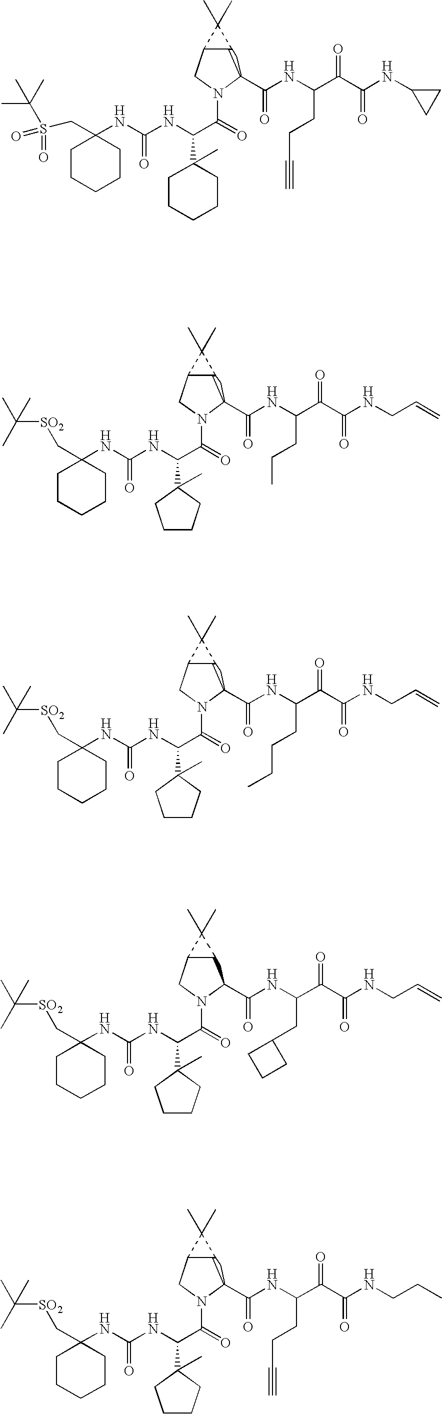 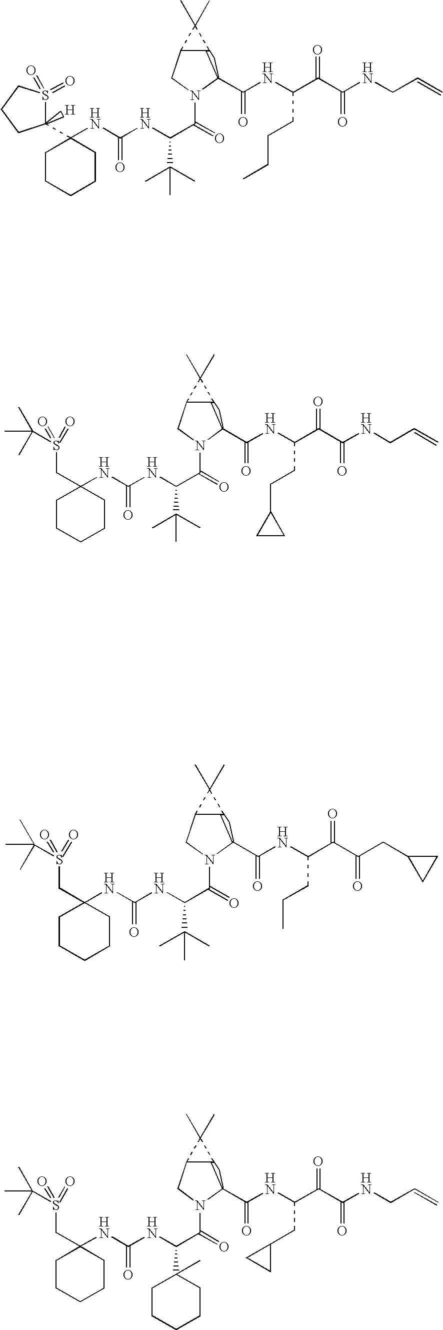 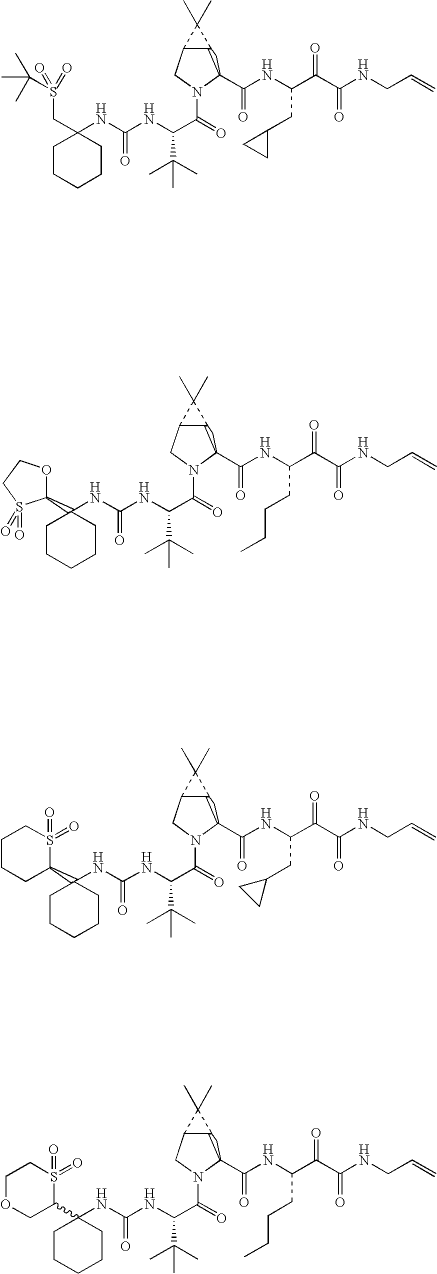 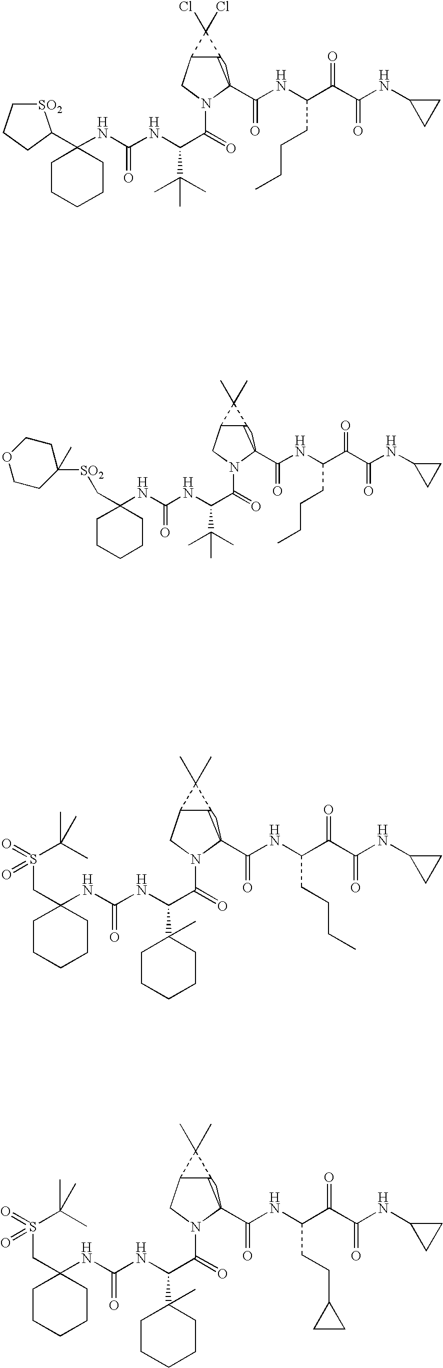 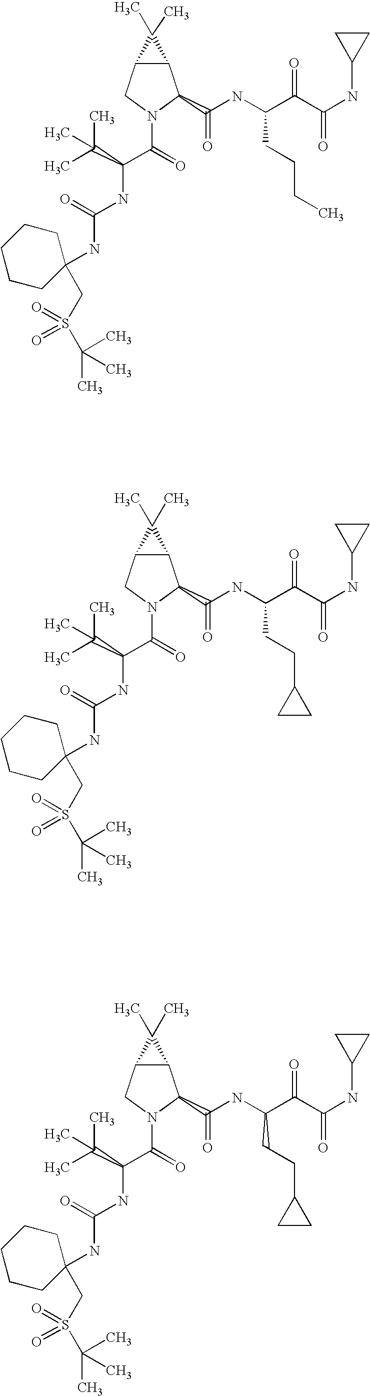
and or a pharmaceutically acceptable salt, solvate or ester thereof.

9. A method of inhibiting cathepsin activity in a subject in need thereof comprising administering to said subject an effective amount of at least one compound of formula XX: 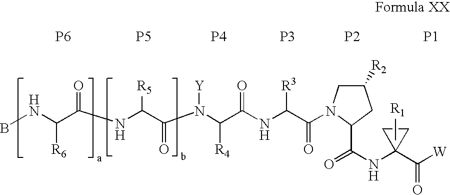
or a pharmaceutically acceptable salt, solvate or ester thereof; wherein: a is 0 or 1; b is 0 or 1; Y is H or C₁₋₆alkyl;
B is H, an acyl derivative of formula R₇-C(O)— or a sulfonyl of formula R₇—SO2 wherein
R7 is (i) C₁₋₁₀ alkyl optionally substituted with carboxyl, C₁₋₆ alkanoyloxy or C₁₋₆ alkoxy;
(ii) C₃₋₇ cycloalkyl optionally substituted with carboxyl, (C₁₋₆ alkoxy)carbonyl or phenylmethoxycarbonyl;
(iii) C₆ or C₁₀ aryl or C₇₋₁₆ aralkyl optionally substituted with C₁₋₆ alkyl, hydroxy, or amino optionally substituted with C₁₋₆ alkyl; or
(iv) Het optionally substituted with C₁₋₆ alkyl, hydroxy, amino optionally substituted with C₁₋₆ alkyl, or amido optionally substituted with C₁₋₆ alkyl;
R₆, when present, is C₁₋₆ alkyl substituted with carboxyl;
R₅, when present, is C₁₋₆ alkyl optionally substituted with carboxyl;
R₄ is C₁₋₁₀ alkyl, C₃₋₇ cycloalkyl or C₄₋₁₀ (alkylcycloalkyl);
R₃ is C₁₋₁₀ alkyl, C₃₋₇ cycloalkyl or C₄₋₁₀ (alkylcycloalkyl);
R₂ is CH₂—R₂₀, NH—R₂₀, O—R₂₀ or S—R₂₀, wherein R₂₀ is a saturated or unsaturated C₃₋₇ cycloalkyl or C₄₋₁₀ (alkyl cycloalkyl) being optionally mono-, di- or tri-substituted with R₂₁, or R₂₀ is a C₆ or C₁₀ aryl or C₇₋₁₆ aralkyl optionally mono-, di- or tri- substituted with R₂₁,
or R₂₀ is Het or (lower alkyl)-Het optionally mono-, di- or tri- substituted with R₂₁, wherein each R₂₁ is independently C₁₋₆ alkyl; C₁₋₆alkoxy; amino optionally mono- or di-substituted with C₁₋₆ alkyl; sulfonyl; N0₂; OH; SH; halo; haloalkyl; amido optionally mono-substituted with C₁₋₆ alkyl, C₆ or C₁₀ aryl, C₇₋₁₆aralkyl, Het or (lower alkyl)-Het; carboxyl; carboxy(lower alkyl); C₆ or C₁₀ aryl, C₇₋₁₆aralkyl or Het, said aryl, aralkyl or Het being optionally substituted with R₂₂;
wherein R₂₂ is C₁₆alkyl; C₁₋₆ alkoxy; amino optionally mono- or di- substituted with C₁₋₆ alkyl; sulfonyl; N0₂; OH; SH; halo; haloalkyl; carboxyl; amide or (lower alkyl)amide;
R₁ is C₁₋₆ alkyl or C₂₋₆ alkenyl optionally substituted with halogen; and
W is hydroxy or a N-substituted amino.

10. A method of inhibiting cathepsin activity in a subject in need thereof comprising administering to said subject an effective amount of at least one compound of formula XXI: 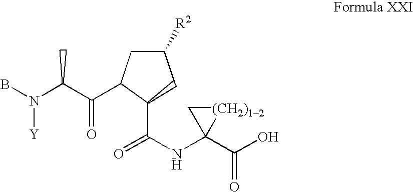
or a pharmaceutically acceptable salt, solvate or ester thereof; wherein:
B is H, a C₆ or C₁₀ aryl, C₇₋₁₆ aralkyl; Het or (lower alkyl)- Het, all of which optionally substituted with C₁₋₆ alkyl; C₁₋₆ alkoxy; C₁₋₆ alkanoyl; hydroxy; hydroxyalkyl; halo; haloalkyl; nitro; cyano; cyanoalkyl; amino optionally substituted with C₁₋₆ alkyl; amido; or (lower alkyl)amide;
or B is an acyl derivative of formula R₄—C(O)—; a carboxyl of formula R₄-0-C(O)—; an amide of formula R₄—N(R₅)—C(O)—; a thioamide of formula R₄—N(R₅)—C(S)—; or a sulfonyl of formula R₄—SO2 wherein
R₄ is (i) C₁₋₁₀ alkyl optionally substituted with carboxyl, C₁₋₆ alkanoyl, hydroxy, C₁₋₆ alkoxy, amino optionally mono- or di-substituted with C₁₋₆ alkyl, amido, or (lower alkyl) amide;
(ii) C₃₋₇ cycloalkyl, C₃₋₇ cycloalkoxy, or C₄₋₁₀ alkylcycloalkyl, all optionally substituted with hydroxy, carboxyl, (C₁₋₆ alkoxy)carbonyl, amino optionally mono- or di-substituted with C₁₋₆ alkyl, amido, or (lower alkyl) amide;
(iii) amino optionally mono- or di-substituted with C₁₋₆ alkyl; amido; or (lower alkyl)amide;
(iv) C₆ or C₁₀ aryl or C₇₋₁₆ aralkyl, all optionally substituted with C₁₋₆ alkyl, hydroxy, amido, (lower alkyl)amide, or amino optionally mono- or di- substituted with C₁₋₆ alkyl; or
(v) Het or (lower alkyl)-Het, both optionally substituted with C₁₋₆ alkyl, hydroxy, amido, (lower alkyl) amide, or amino optionally mono- or di-substituted with C₁₋₆ alkyl;
R₅ is H or C₁₋₆ alkyl;
with the proviso that when R₄ is an amide or a thioamide, R₄ is not (ii) a cycloalkoxy;
Y is H or C₁₋₆ alkyl;
R₃ is C₁₋₈ alkyl, C₃₋₇ cycloalkyl, or C₄₋₁₀ alkylcycloalkyl, all optionally substituted with hydroxy, C₁₋₆ alkoxy, C₁₋₆ thioalkyl, amido, (lower alkyl)amido, C₆ or C₁₀ aryl, or C₇₋₁₆ aralkyl;
R₂ is CH₂—R₂₀, NH—R₂₀, O—R₂₀ or S—R₂₀, wherein R₂₀ is a saturated or unsaturated C₃₋₇ cycloalkyl or C₄₋₁₀ (alkylcycloalkyl), all of which being optionally mono-, di- or tri-substituted with R₂₁, or R₂₀ is a C₆ or C₁₀ aryl or C₇₋₁₄ aralkyl, all optionally mono-, di- or tri-substituted with R₂₁,
or R₂₀ is Het or (lower alkyl)-Het, both optionally mono-, di- or tri- substituted with R₂₁,
wherein each R₂₁ is independently C₁₋₆ alkyl; C₁₋₆ alkoxy; lower thioalkyl; sulfonyl; N0₂; OH; SH; halo; haloalkyl; amino optionally mono- or di- substituted with C₁₋₆ alkyl, C₆ or C₁₀ aryl, C₇₋₁₄ aralkyl, Het or (lower alkyl)-Het; amido optionally mono-substituted with C₁₋₆ alkyl, C₆ or C₁₀ aryl, C₇₋₁₄ aralkyl, Het or (lower alkyl)-Het; carboxyl; carboxy(lower alkyl); C₆ or C₁₀ aryl, C₇₋₁₄ aralkyl or Het, said aryl, aralkyl or Het being optionally substituted with R₂₂;
wherein R₂₂ is C₁₋₆ alkyl; C₃₋₇ cycloalkyl; C₁₋₆ alkoxy; amino optionally mono- or di-substituted with C₁₋₆ alkyl; sulfonyl; (lower alkyl)sulfonyl; N0₂; OH; SH; halo; haloalkyl; carboxyl; amide; (lower alkyl)amide; or Het optionally substituted with C₁₋₆ alkyl;
R1 is H; C₁₋₆ alkyl, C₃₋₇ cycloalkyl, C₂₋₆ alkenyl, or C₂₋₆ alkynyl, all optionally substituted with halogen.

11. A method of inhibiting cathepsin activity in a subject in need thereof comprising administering to said subject an effective amount of at least one compound of formula XXII: 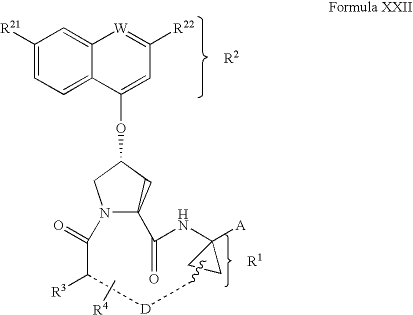
or a pharmaceutically acceptable salt, solvate or ester thereof; wherein
W is CH or N,
R²¹ is H, halo, C₁₋₆ alkyl, C₃₋₆ cycloalkyl, C₁₋₆ haloalkyl, C₁₋₆ alkoxy, C₃₋₆ cycloalkoxy, hydroxy, or N(R²³)₂ , wherein each R²³ is independently H, C₁₋₆ alkyl or C₃₋₆ cycloalkyl;
R²² is H, halo, C₁₋₆ alkyl, C₃₋₆ cycloalkyl, C₁₋₆ haloalkyl, C₁₋₆ thioalkyl, C₁₋₆ alkoxy, C₃₋₆ cycloalkoxy, C₂₋₇ alkoxyalkyl, C₃₋₆ cycloalkyl, C_{6 or 10} aryl or Het, wherein Het is a five-, six-, or seven-membered saturated or unsaturated heterocycle containing from one to four heteroatoms selected from nitrogen, oxygen and sulfur;
said cycloalkyl, aryl or Het being substituted with R²⁴, wherein R²⁴ is H, halo, C₁₋₆ alkyl, C₃₋₆ cycloalkyl, C₁₋₆ alkoxy, C₃₋₆ cycloalkoxy, NO₂, N(R²⁵)₂, NH—C(O)—R²⁵ or NH—C(O)—NH—R²⁵, wherein each R²⁵ is independently: H, C₁₋₆ alkyl or C₃₋₆ cycloalkyl;
or R²⁴ is NH—C(O)—OR²⁶ wherein R²⁶ is C₁₋₆ alkyl or C₃₋₆ cycloalkyl;
R³ is hydroxy, NH₂, or a group of formula —NH—R³¹, wherein R³¹ is C_{6 or 10} aryl, heteroaryl, —C(O)—R³², —C(O)—NHR³² or —C(O)—OR³², wherein R³² is C₁₋₆ alkyl or C₃₋₆ cycloalkyl;
D is a 5 to 10-atom saturated or unsaturated alkylene chain optionally containing one to three heteroatoms independently selected from: O, S, or N—R⁴¹ , wherein
R⁴¹ is H, C₁₋₆ alkyl, C₃₋₆ cycloalkyl or —C(O)—R⁴²,
wherein R⁴² is C₁₋₆ alkyl, C₃₋₆ cycloalkyl or C_{6 or 10} aryl;
R⁴ is H or from one to three substituents at any carbon atom of said chain D, said substituent independently selected from the group consisting of: C₁₋₆ alkyl, C₁₋₆ haloalkyl, C₁₋₆ alkoxy, hydroxy, halo, amino, oxo, thio and C 1-6 thioalkyl, and A is an amide of formula —C(O)—NH—R⁵, wherein R⁵ is selected from the group consisting of: C₁₋₈ alkyl, C₃₋₆ cycloalkyl, C_{6 or 10} aryl and C₇₋₁₆ aralkyl;
or A is a carboxylic acid.

12. A method of inhibiting cathepsin activity in a subject in need thereof comprising administering to said subject an effective amount of at least one compound of formula XXIV: 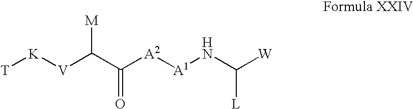
or a pharmaceutically acceptable salt, solvate or ester thereof; wherein: W is: 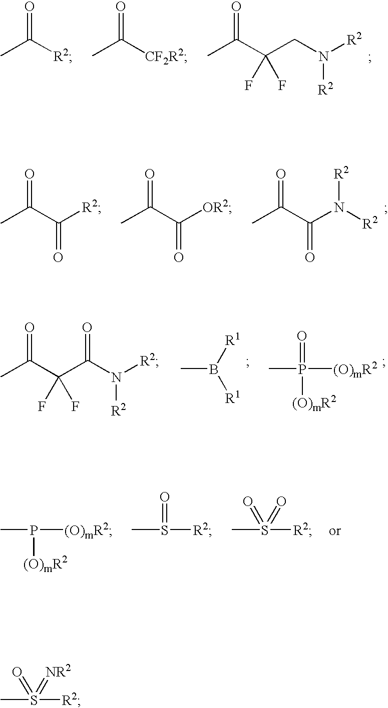
m is 0 or 1;
each R¹ is hydroxy, alkoxy, or aryloxy, or each R¹ is an oxygen atom and together with the boron, to which they are each bound, form a 5-7 membered ring, wherein the ring atoms are carbon, nitrogen, or oxygen;
each R² is independently hydrogen, alkyl, alkenyl, aryl, aralkyl, aralkenyl, cycloalkyl, cycloalkylalkyl, cycloalkenyl, cycloalkenylalkyl, heterocyclyl, heterocyclylalkyl, heterocyclylalkenyl, heteroaryl, or heteroaralkyl, or two R² groups, which are bound to the same nitrogen atom, form together with that nitrogen atom, a 5-7 membered monocyclic heterocyclic ring system; wherein any R² carbon atom is optionally substituted with J;
J is alkyl, aryl, aralkyl, alkoxy, aryloxy, aralkoxy, cycloalkyl, cycloalkoxy, heterocyclyl, heterocyclyloxy, heterocyclylalkyl, keto, hydroxy, amino, alkylamino, alkanoylamino, aroylamino, aralkanoylamino, carboxy, carboxyalkyl, carboxamidoalkyl, halo, cyano, nitro, formyl, acyl, sulfonyl, or sulfonamido and is optionally substituted with 1-3 J¹ groups;
J¹ is alkyl, aryl, aralkyl, alkoxy, aryloxy, heterocyclyl, heterocyclyloxy, keto, hydroxy, amino, alkanoylamino, aroylamino, carboxy, carboxyalkyl, carboxamidoaikyl, halo, cyano, nitro, formyl, sulfonyl, or sulfonamido;
L is alkyl, alkenyl, or alkynyl, wherein any hydrogen is optionally substituted with halogen, and wherein any hydrogen or halogen atom bound to any terminal carbon atom is optionally substituted with sulfhydryl or hydroxy;
A¹ is a bond;
R⁴ is alkyl, cycloalkyl, aryl, aralkyl, heterocyclyl, heterocyclylalkyl, heteroaryl, heteroaralkyl, carboxyalkyl, or carboxamidoalkyl, and is optionally substituted with 1-3 J groups;
R⁵ and R⁵ are independently hydrogen, alkyl, alkenyl, aryl, aralkyl, aralkenyl, cycloalkyl, cycloalkylalkyl, cycloalkenyl, heterocyclyl, heterocyclylalkyl, heteroaryl, or heteroaralkyl, and is optionally substituted with 1-3 J groups;
X is a bond, —C(H)(R7)-, -0-, —S—, or —N(R8)-;
R⁷ is hydrogen, alkyl, alkenyl, aryl, aralkyl, heterocyclyl, heterocyclylalkyl, heteroaryl, or heteroaralkyl, and is optionally substititued with 1-3 J groups;
R⁸ is hydrogen alkyl, aryl, aralkyl, heterocyclyl, heterocyclylalkyl, heteroaryl, heteroaralkyl, aralkanoyl, heterocyclanoyl, heteroaralkanoyl, —C(O)R¹⁴, —S0₂R¹⁴, or carboxamido, and is optionally substititued with 1-3 J groups; or R⁸ and Z, together with the atoms to which they are bound, form a nitrogen containing mono- or bicyclic ring system optionally substituted with 1-3 J groups;
R¹⁴ is alkyl, aryl, aralkyl, heterocyclyl, heterocyclyalkyl, heteroaryl, or heteroaralkyl;
Y is a bond, —CH₂—, —C(O)—, —C(O)C(O)—, —S(O)—, —S(0)₂-, or —S(O)(NR⁷)—, wherein R⁷ is as defined above;
Z is alkyl, aryl, aralkyl, cycloalkyl, cycloalkylalkyl, heterocyclyl, heterocyclylalkyl, heteroaryl, heteroaralkyl, —OR², or —N(R²)₂, wherein any carbon atom is optionally substituted with J, wherein R² is as defined above;
A²is a bond or 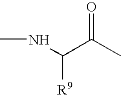
R⁹ is alkyl, cycloalkyl, aryl, aralkyl, heterocyclyl, heterocyclylalkyl, heteroaryl, heteroaralkyl, carboxyalkyl, or carboxamidoalkyl, and is optionally substituted with 1-3 J groups;
M is alkyl, cycloalkyl, aryl, aralkyl, heterocyclyl, heterocyclylalkyl, heteroaryl, or heteroaralkyl, optionally substituted by 1-3 J groups, wherein any alkyl carbon atom may be replaced by a heteroatom;
V is a bond, —CH₂—, —C(H)(R¹¹)—, -0-, —S—, or —N(R¹¹)—;
R¹¹ is hydrogen or C₁₋₃ alkyl;
K is a bond, -0-, —S—, —C(O)—, —S(O)—, -S(0)2-, or -S(O)(NR¹¹)—, wherein R¹¹ is as defined above;
T is —R¹², -alkyl-R¹², -alkenyl-R¹², -alkynyl-R¹², —OR¹², —N(R¹²)2, —C(O)R¹², —C(═NOalkyl)R¹², or 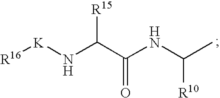
R¹² is hydrogen, aryl, heteroaryl, cycloalkyl, heterocyclyl, cycloalkylidenyl, or heterocycloalkylidenyl, and is optionally substituted with 1-3 J groups, or a first R¹² and a second R¹², together with the nitrogen to which they are bound, form a mono- or bicyclic ring system optionally substituted by 1-3 J groups;
R¹⁰ is alkyl, cycloalkyl, aryl, aralkyl, heterocyclyl, heterocyclylalkyl, heteroaryl, heteroaralkyl, carboxyalkyl, or carboxamidoalkyl, and is optionally substituted with 1-3 hydrogens J groups;
R¹⁵ is alkyl, cycloalkyl, aryl, aralkyl, heterocyclyl, heterocyclylalkyl, heteroaryl, heteroaralkyl, carboxyalkyl, or carboxamidoalkyl, and is optionally substituted with 1-3 J groups; and
R¹⁶ is hydrogen, alkyl, aryl, heteroaryl, cycloalkyl, or heterocyclyl.

13. A method of inhibiting cathepsin activity in a subject in need thereof comprising administering to said subject an effective amount of at least one compound of formula XXVI: 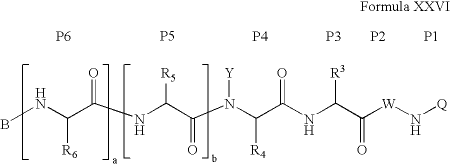
or a pharmaceutically acceptable salt, solvate or ester thereof; wherein
B is an acyl derivative of formula R₁₁—C(O)— wherein R₁₁ is CI-10 alkyl optionally substituted with carboxyl; or R₁₁ is C₆ or C₁₀ aryl or C₇₋₁₆ aralkyl optionally substituted with a C₁₋₆ alkyl;
a is 0 or 1;
R₆, when present, is carboxy(lower)alkyl;
b is 0 or 1;
R₅, when present, is C₁₋₆ alkyl, or carboxy(lower)alkyl;
Y is H or C₁₋₆ alkyl;
R₄ is C₁₋₁₀ alkyl; C₃₋₁₀ cycloalkyl;
R₃ is C1-10 alkyl; C₃₋₁₀ cycloalkyl;
W is a group of formula: 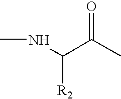
wherein R₂ is C₁₋₁₀ alkyl or C₃₋₇ cycloalkyl optionally substituted with carboxyl;
C₆ or C₁₀ aryl; or C₇₋₁₆ aralkyl; or
W is a group of formula: 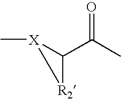
wherein X is CH or N; and
R₂′ is C₃₋₄ alkylene that joins X to form a 5- or 6-membered ring, said ring optionally substituted with OH; SH; NH2; carboxyl; R₁₂; OR₁₂, SR₁₂, NHR₁₂ or NR₁₂R₁₂′ wherein R₁₂ and R₁₂′ are independently:
cyclic C₃₋₁₆ alkyl or acyclic C₁₋₁₆ alkyl or cyclic C₃₋₁₆ alkenyl or acyclic C₂₋₁₆ alkenyl, said alkyl or alkenyl optionally substituted with NH₂, OH, SH, halo, or carboxyl; said alkyl or alkenyl optionally containing at least one heteroatom selected independently from the group consisting of: 0, S, and N; or
R₁₂ and R₁₂′ are independently C₆ or C₁₀ aryl or C₇₋₁₆ aralkyl optionally substituted with C₁₋₆ alkyl, NH₂, OH, SH, halo, carboxyl or carboxy(lower)alkyl; said aryl or aralkyl optionally containing at least one heteroatom selected independently from the group consisting of: 0, S, and N;
said cyclic alkyl, cyclic alkenyl, aryl or aralkyl being optionally fused with a second 5-, 6-, or 7-membered ring to form a cyclic system or heterocycle, said second ring being optionally substituted with NH₂. OH, SH, halo, carboxyl or carboxy(lower)alkyl; C₆ or C₁₀ aryl, or heterocycle; said second ring optionally containing at least one heteroatom selected independently from the group consisting of: 0, S, and N;
Q is a group of the formula: 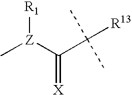
wherein Z is CH or N;
X is 0 or S;
R₁ is H, C₁₋₆ alkyl or C₁₋₆ alkenyl both optionally substituted with thio or halo; and
when Z is CH, then R₁₃ is H; CF₃; CF₂CF₃; CH₂—R₁₄; CH(F)—R₁₄; CF₂—R₁₄; NR₁₄R₁₄′; S—R₁₄; or C0-NH—R₁₄ wherein R₁₄ and R₁₄′ are independently hydrogen, cyclic C₃₋₁₀ alkyl or acyclic C₁₋₁₀ alkyl or cyclic C₃₋₁₀ alkenyl or acyclic C₂₋₁₀ alkenyl, said alkyl or alkenyl optionally substituted with NH₂, OH, SH, halo or carboxyl; said alkyl or alkenyl optionally containing at least one heteroatom selected independently from the group consisting of: 0, S, and N; or
R₁₄ and R₁₄′ are independently C₆ or C₁₀ aryl or C₇₋₁₆ aralkyl optionally substituted with C₁₋₆ alkyl, NH₂, OH, SH, halo, carboxyl or carboxy(lower)alkyl or substituted with a further C₃₋₇ cycloalkyl, C₆ or C₁₀ aryl, or heterocycle; said aryl or aralkyl optionally containing at least one heteroatom selected independently from the group consisting of: 0, S, and N;
said cyclic alkyl, cyclic alkenyl, aryl or aralkyl being optionally fused with a second 5-, 6-, or 7-membered ring to form a cyclic system or heterocycle, said second ring being optionally substituted with NH₂, OH, SH, halo, carboxyl or carboxy(lower)alkyl or substituted with a further C₃₋₇ cycloalkyl, C₆ or C₁₀ aryl, or heterocycle; said second ring optionally containing at least one heteroatom selected independently from the group consisting of: 0, S, and N;
or R₁₄ and R₁₄′ are independently C₁₋₄ alkyl which when joined together with N form a 3 to 6-membered nitrogen-containing ring which is optionally fused with a further C₃₋₇ cycloalkyl, C₆ or C₁₀ aryl or heterocycle;
with the proviso that when Z is CH, then R₁₃ is not an α-amino acid or an ester thereof;
when Z is N, then R₁₃ is H; carboxy; C₁₋₆ alkyl optionally substituted with carboxy; CH₂—R₁₄; CHR₁₄R₁₄′; CH(F)—R₁₄; O—R₁₄; NR₁₄R₁₄′ or S—R₁₄ wherein R₁₄ and R₁₄′ are as defined above; or
Q is a phosphonate group of the formula: 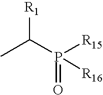
wherein R₁₅ and R₁₆ are independently C₆₋₂₀ aryloxy; and R₁ is as defined above.

14. A method of inhibiting cathepsin activity in a subject in need thereof comprising administering to said subject an effective amount of at least one compound selected from the group consisting of: 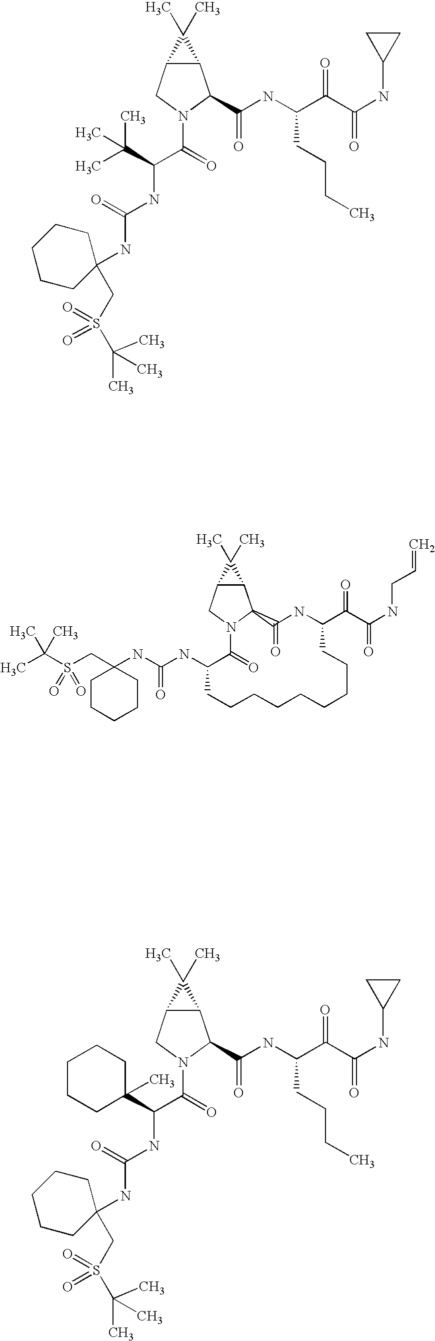 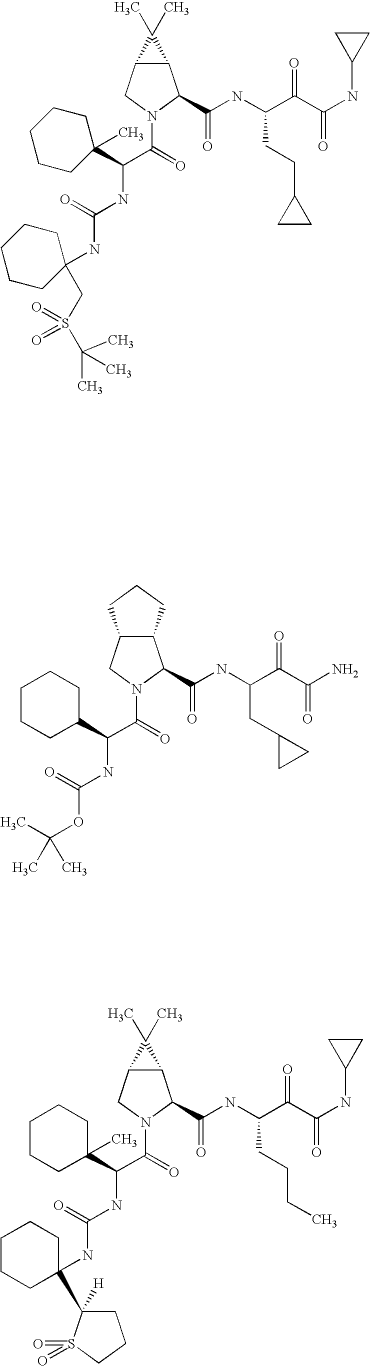 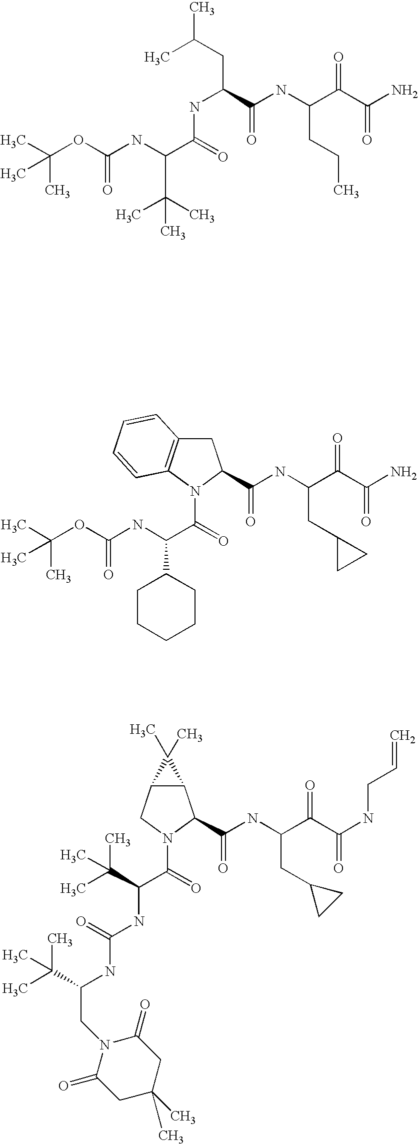 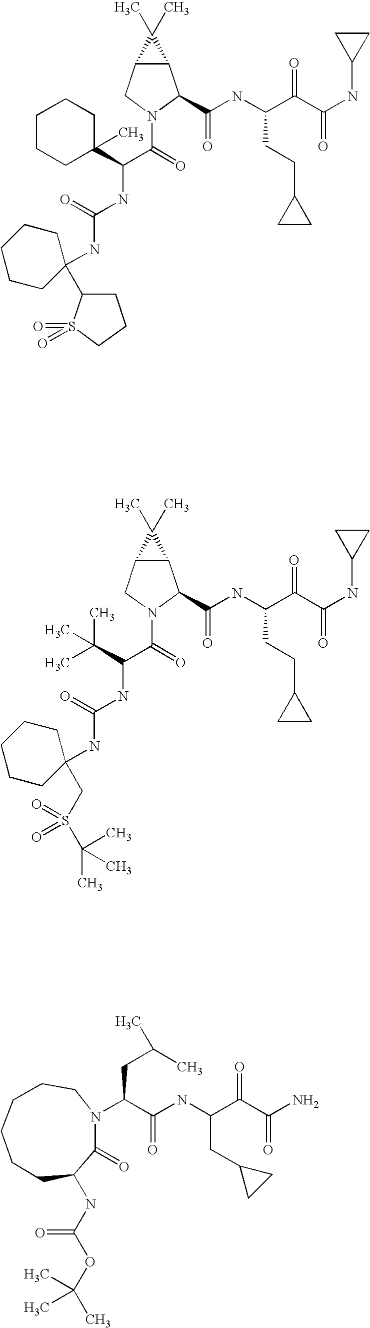 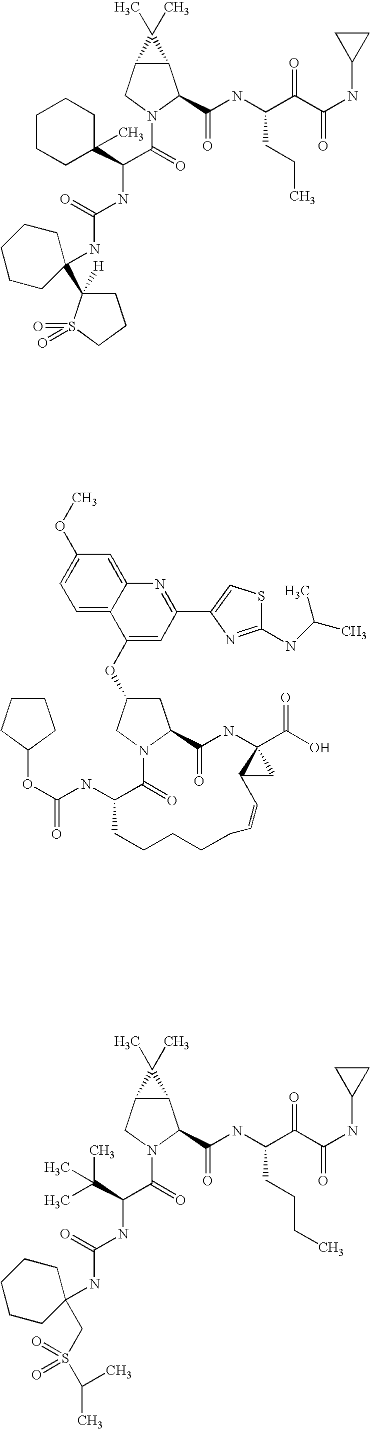 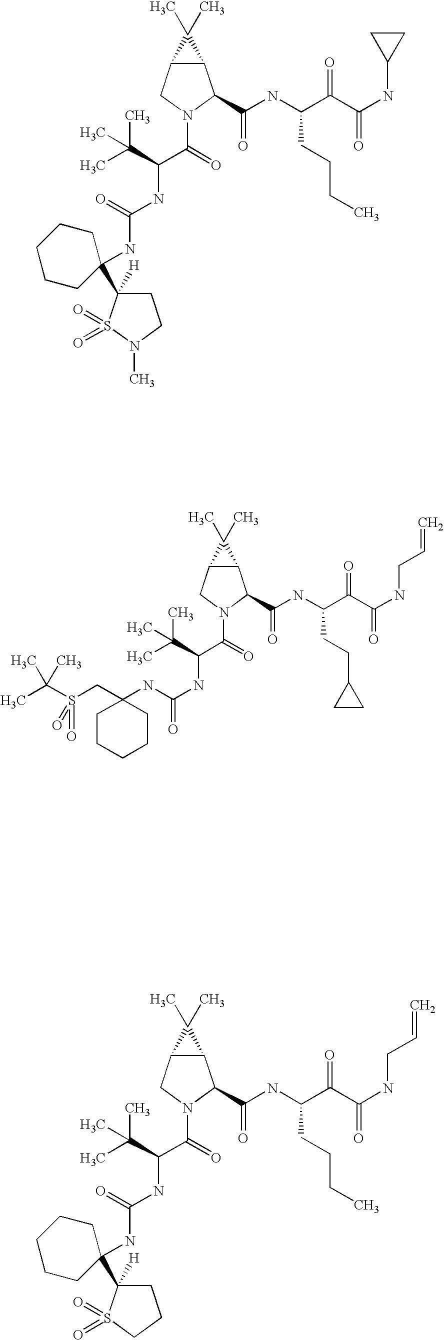 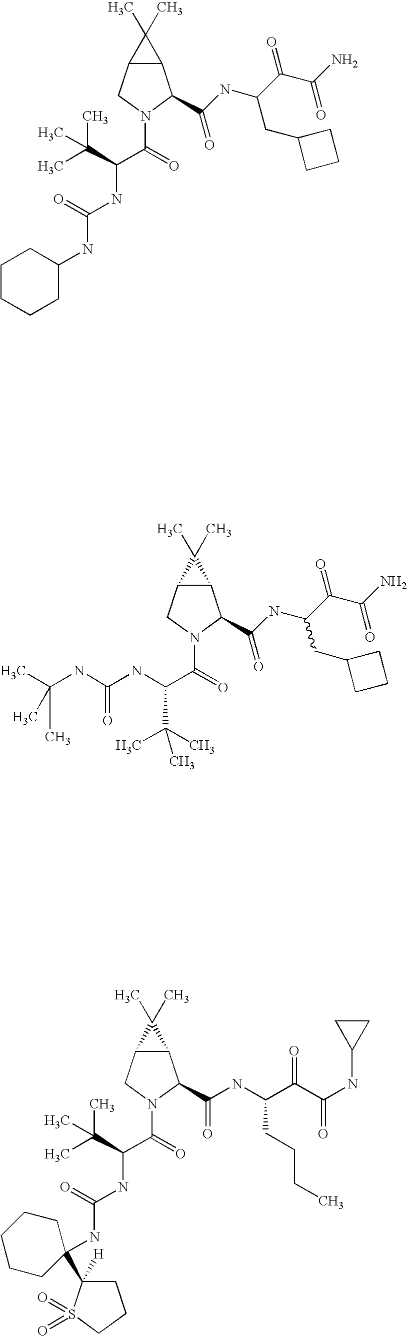 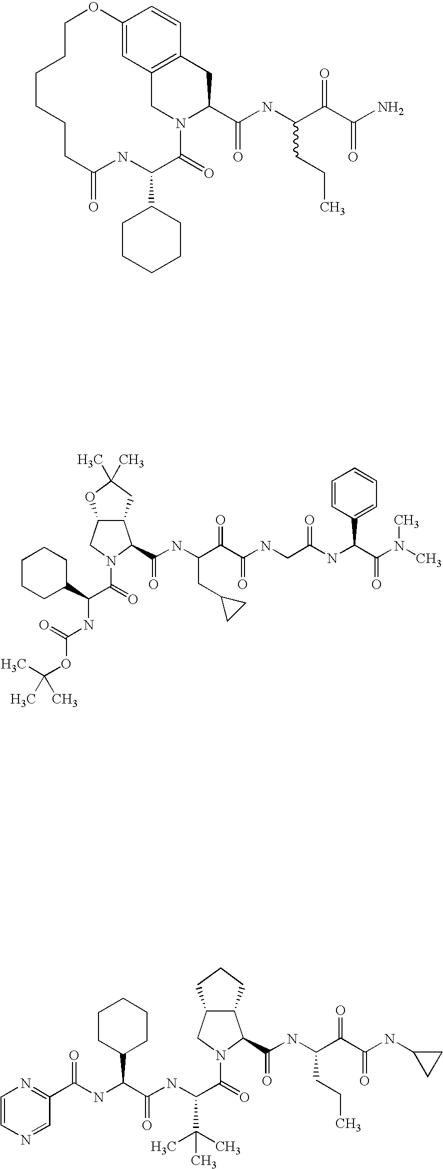 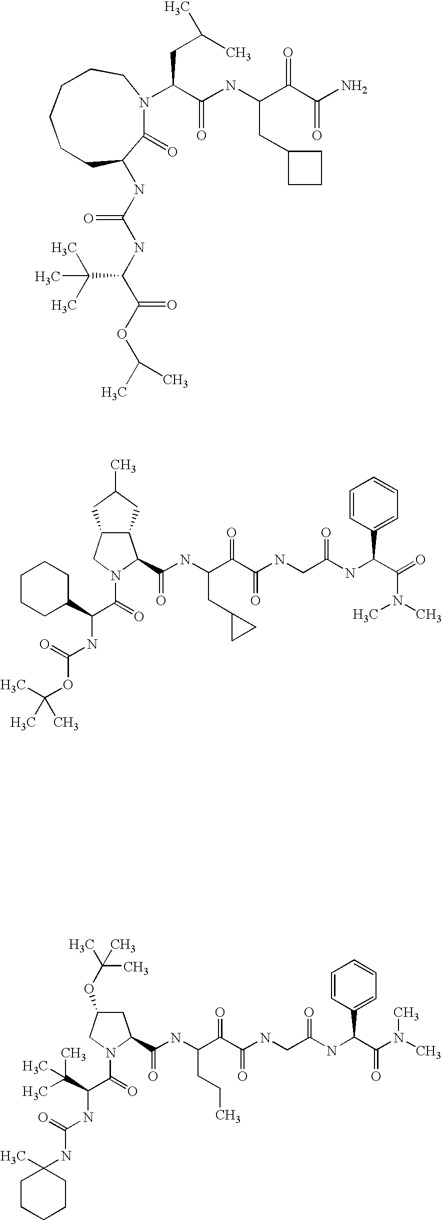 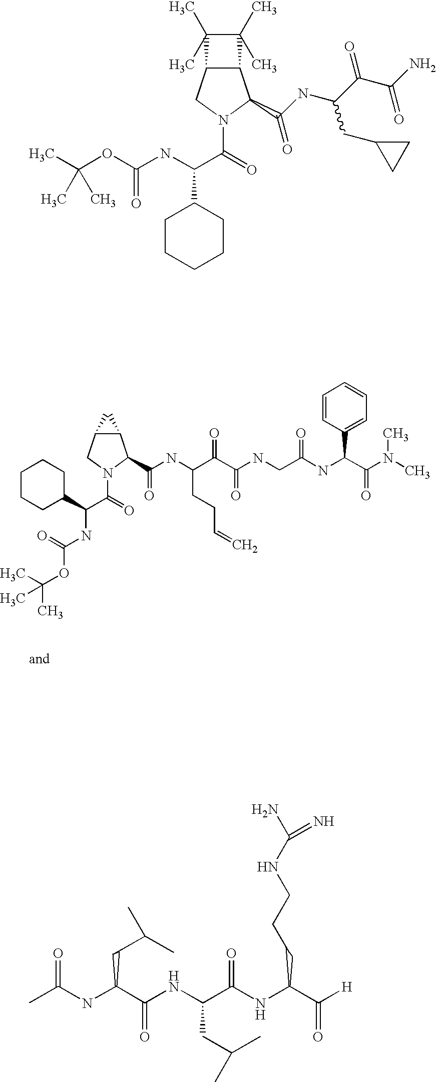
or a pharmaceutically acceptable salt, solvate or ester thereof.

15. A method of treating a disease selected from the group consisting of cellular proliferative disease, inflammatory diseases, cardiovascular diseases, central nervous system diseases, diseases characterized by bone loss, gingival diseases, and diseases characterized by excessive cartilage or matrix degradation, in a subject comprising administering to said subject in need of such treatment an effective amount of at least one compound of claims 1, 3, 5, 7, 9, 10, 11, 12, 13, or 14, a pharmaceutically acceptable salt, solvate or ester thereof.

16. The method of claim 14, wherein the cellular proliferative disease is selected from the group consisting of cancer, hyperplasia, cardiac hypertrophy, autoimmune diseases, fungal disorders, arthritis, graft rejection, inflammatory bowel disease, immune disorders, inflammation, and cellular proliferation induced after medical procedures.

17. The method of claim 14, wherein the inflammatory disease is selected from the group consisting of organ transplant rejection, graft v. host disease, arthritis, rheumatoid arthritis, inflammatory bowel disease, atopic dermatitis, psoriasis, asthma, allergies, multiple sclerosis, fixed drug eruptions, cutaneous delayed-type hypersentitivity responses, tuberculoid leprosy, type I diabetes, and viral meningitis.

18. The method of claim 14, wherein the central nervous system disease is selected from the group consisting of depression, cognitive function disease, neurodegenerative disease, senile dementia, and psychosis of organic origin.

19. The method of claim 14, wherein the disease characterized by bone loss is osteoporosis.

20. The method of claim 14, wherein the gingival diseases are selected from the group consisting of gingivitis and periodontitis.

21. The method of claim 14, wherein the disease characterized by excessive cartilage or matrix degradation is selected from the group consisting of osteoarthritis and rheumatoid arthritis.

22. The method of claim 15, wherein the cancer is a cancer selected from the group consisting of cancers of the brain, genitourinary tract, cardiac, gastrointestine, liver, bone, nervous system, and lung.

23. The method of claim 15, wherein the cancer is selected from the group consisting of lung adenocarcinama, small cell lung cancer, pancreatic cancer, and breast carcinoma.

24. The method of claim 15, further comprising radiation therapy.

25. The method of claim 15, further comprising administering to the subject at least one compound selected from the group consisting of an anti-cancer agent, a PPAR-γ agonist, a PPAR-δ agonist, an inhibitor of inherent multidrug resistance, an anti-emetic agent, and an immunologic-enhancing drug.

26. The method of claim 15, wherein the disease is cancer.

27. The method of claim 25, further comprising radiation therapy.

28. The method of claim 24, wherein the anti-cancer agent is selected from the group consisting of an estrogen receptor modulator, an androgen receptor modulator, retinoid receptor modulator, a cytotoxic/cytostatic agent, an antiproliferative agent, a prenyl-protein transferase inhibitor, an HMG-CoA reductase inhibitor, an angiogenesis inhibitor, an inhibitor of cell proliferation and survival signaling, an agent that interferes with a cell cycle checkpoint, and an apoptosis inducing agent.

29. The method of claim 27, further comprising one or more anti-cancer agent selected from the group consisting of cytostatic agent, cytotoxic agent, taxane, topoisomerase II inhibitor, topoisomerase I inhibitor, tubulin interacting agent, hormonal agent, thymidilate synthase inhibitor, anti-metabolite, alkylating agent, farnesyl protein transferase inhibitor, signal transduction inhibitor, EGFR kinase inhibitor, antibody to EGFR, C-abl kinase inhibitor, hormonal therapy combination, and aromatase combination.

30. The method of claim 14, further comprising one or more agents selected from the group consisting of Uracil mustard, Chlormethine, Ifosfamide, Melphalan, Chlorambucil, Pipobroman, Triethylenemelamine, Triethylenethiophosphoramine, Busulfan, Carmustine, Lomustine, Streptozocin, Dacarbazine, Floxuridine, Cytarabine, 6-Mercaptopurine, 6-Thioguanine, Fludarabine phosphate, oxaliplatin, leucovirin, oxaliplatin, Pentostatine, Vinblastine, Vincristine, Vindesine, Bleomycin, Dactinomycin, Daunorubicin, Doxorubicin, Epirubicin, Idarubicin, Mithramycin, Deoxycoformycin, Mitomycin-C, L-Asparaginase, Teniposide 17α-Ethinylestradiol, Diethylstilbestrol, Testosterone, Prednisone, Fluoxymesterone, Dromostanolone propionate, Testolactone, Megestrolacetate, Methylprednisolone, Methyltestosterone, Prednisolone, Triamcinolone, Chlorotrianisene, Hydroxyprogesterone, Aminoglutethimide, Estramustine, Medroxyprogesteroneacetate, Leuprolide, Flutamide, Toremifene, goserelin, Cisplatin, Carboplatin, Hydroxyurea, Amsacrine, Procarbazine, Mitotane, Mitoxantrone, Levamisole, Navelbene, Anastrazole, Letrazole, Capecitabine, Reloxafine, Droloxafine, Hexamethylmelamine, doxorubicin, cyclophosphamide, gemcitabine, interferons, pegylated interferons, Erbitux and mixtures thereof.

31. The method of claim 14, wherein the disease is at least one selected from the group consisting of cancer, rheumatoid arthritis and osteoporosis.
